(19) United States
(12) Patent Application Publication
(10) Pub. No.: US 2024/0059710 A1
(43) Pub. Date: Feb. 22, 2024

(54) KRAS G12D Inhibitors

(71) Applicant: Jacobio Pharmaceuticals Co., Ltd., Beijing (CN)

(72) Inventors: Huifeng HAN, Beijing (CN); Panliang GAO, Beijing (CN); Wenlong ZHANG, Beijing (CN); Cunbo MA, Beijing (CN); Peng WANG, Beijing (CN); Dan LIU, Beijing (CN); Hao ZHANG, Beijing (CN); Wei LONG, Beijing (CN)

(21) Appl. No.: 18/037,801

(22) PCT Filed: Nov. 19, 2021
(86) PCT No.: PCT/CN2021/131690

§ 371 Date: May 19, 2023

(30) Foreign Application Priority Data
Nov. 20, 2020 (WO) PCT/CN2020/130351
Mar. 23, 2021 (WO) PCT/CN2021/082498
Mar. 30, 2021 (WO) PCT/CN2021/083768
May 8, 2021 (WO) PCT/CN2021/092466
Jun. 11, 2021 (WO) PCT/CN2021/099745
Jun. 11, 2021 (WO) PCT/CN2021/099750
Jun. 24, 2021 (WO) PCT/CN2021/102166
Jun. 24, 2021 (WO) PCT/CN2021/102172
Sep. 30, 2021 (WO) PCT/CN2021/122046

(51) Int. Cl. C07D 519/00; C07D 487/08; A61P 35/00

(52) U.S. Cl. CPC C07D 519/00; C07D 487/08; A61P 35/00

(57) ABSTRACT

KRAS G12D inhibitors of Formula (I), a composition containing the inhibitor and the use thereof.

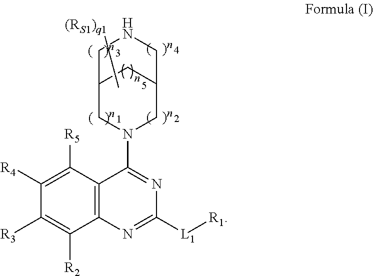

### CROSS-REFERENCES TO RELATED APPLICATIONS

This application claims priority to PCT/CN2020/130351, filed Nov. 20, 2020; PCT/CN2021/082498, filed Mar. 23, 2021; PCT/CN2021/083768, filed Mar. 30, 2021; PCT/CN2021/092466, filed May 8, 2021; PCT/CN2021/099745, filed Jun. 11, 2021; PCT/CN2021/099750, filed Jun. 11, 2021; PCT/CN2021/102166, filed Jun. 24, 2021; PCT/CN2021/102172, filed Jun. 24, 2021; and PCT/CN2021/122046, filed Sep. 30, 2021, each of which is incorporated herein in its entirety and for all purposes.

### TECHNICAL FIELD

The invention relates to KRAS G12D (glycine12 is mutated to aspartic acid) inhibitors, a composition containing the inhibitor and the use thereof.

### BACKGROUND ART

RAS represents a population of 189 amino acid monomeric globular proteins (21 kDa molecular weight) that are associated with the plasma membrane and bind to GDP or GTP, and RAS acts as a molecular switch. When the RAS contains bound GDP, it is in a stationary or closed position and is inactive. When cells are exposed to certain growth-promoting stimuli, RAS is induced to exchange their bound GDP for GTP. In the case of binding to GTP, RAS is open and is capable of interacting with other proteins (its “downstream targets”) and activating the proteins. The RAS protein itself has an inherently low ability to hydrolyze GTP back to GDP, thereby turning itself into a closed state. Closing RAS requires an exogenous protein called GTPase activating protein (GAP) that interacts with RAS and greatly accelerates the conversion of GTP to GDP. Any mutation in RAS that affects its ability to interact with GAP or convert GTP back to GDP will result in prolonged protein activation, and thus conduction to the cell to inform its signaling of continued growth and division. Since these signals cause cell growth and division, over-activated RAS signaling can ultimately lead to cancer.

Structurally, the RAS protein contains a G domain responsible for the enzymatic activity of RAS, guanine nucleotide binding and hydrolysis (GTPase reaction). It also contains a C-terminal extension called the CAAX cassette, which can be post-translationally modified and responsible for targeting the protein to the membrane. The G domain contains a phosphate binding ring (P-ring). The P-loop represents a pocket of a binding nucleotide in a protein, and this is a rigid portion of a domain with conserved amino acid residues necessary for nucleotide binding and hydrolysis (glycine 12, threonine 26 and lysine 16). The G domain also contains a so-called switch I region (residues 30-40) and a switch II region (residues 60-76), both of which are dynamic parts of the protein, since the dynamic portion is converted between stationary and loaded states. The ability is often expressed as a “spring loaded” mechanism. The primary interaction is the hydrogen bond formed by threonine-35 and glycine-60 with the gamma-phosphate of GTP, which maintains the active conformation of the switch I region and the switch II region, respectively. After hydrolysis of GTP and release of phosphate, the two relax into an inactive GDP conformation.

The most notable members of the RAS subfamily are HRAS, KRAS and NRAS, which are primarily involved in many types of cancer. Mutation of any of the three major isoforms of the RAS gene (HRAS, NRAS or KRAS) is one of the most common events in human tumor formation. Approximately 30% of all tumors in human tumors were found to carry some mutations in the RAS gene. It is worth noting that KRAS mutations were detected in 25%-30% of tumors. In contrast, the rate of carcinogenic mutations in NRAS and HRAS family members was much lower (8% and 3%, respectively). The most common KRAS mutations were found at residues G12 and G13 in the P-loop as well as at residue Q61.

With respect to the KRAS G12C inhibitors, some progresses have been taken recently after many years of efforts, for example some promising clinical data have been reported when using Amg-510 and MRT-849 as the therapeutic agent. However, the development of KRAS G12D inhibitors is extraordinarily hard. Thus, there remains a need in the art for improved compounds and methods for treating KRAS G12D mutated cancer. The present invention fulfills this need and provides other related advantages.

### SUMMARY OF INVENTION

[1]. A compound of formula (I), a stereoisomer thereof, an atropisomer thereof, a deuterated derivatives thereof, a tautomer thereof, or a pharmaceutically acceptable salt thereof:

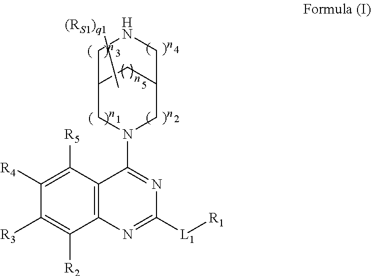

Wherein,
n₁ is selected from 0, 1, 2, 3, 4, 5, or 6; n₂ is selected from 0, 1, 2, 3, 4, 5, or 6; n₃ is selected from 0, 1, 2, 3, 4, 5, or 6; n₄ is selected from 0, 1, 2, 3, 4, 5, or 6; n₅ is selected from 0, 1, 2, 3, 4, 5, or 6;
Each of R_{S1} at each occurrence is independently selected from halogen, —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, —CN, oxo, —N(R₆₁)₂, —OR₆₁, —SR₆₁, —S(═O)R₆₂, —S(═O)₂R₆₂, —C(═O)R₆₂, —C(═O)OR₆₁, —OC(═O)R₆₂, —C(═O)N(R₆₁)₂, —NR₆₁C(═O)R₆₂, —OC(═O)OR₆₁, —NR₆₁C(═O)OR₆₁, —OC(═O)N(R₆₁)₂, —NR₆₁C(═O)N(R₆₁)₂, —S(═O)OR₆₁, —OS(═O)R₆₂, —S(═O)N(R₆₁)₂, —NR₆₁S(═O)R₆₂, —S(═O)₂OR₆₁, —OS(═O)₂R₆₂, —S(═O)₂N(R₆₁)₂, —NR₆₁S(═O)₂R₆₂, —OS(═O)₂OR₆₁, —NR₆₁S(═O)₂OR₆₁, —OS(═O)₂N(R₆₁)₂, —NR₆₁S(═O)₂N(R₆₁)₂, —P(R₆₁)₂, —P(═O)(R₆₂)₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein, said —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is optionally independently substituted with one or more substituents selected from halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —N(R₆₃)₂, —OR₆₃, —SR₆₃, —S(═O)R₆₄, —S(═O)₂R₆₄, —C(═O)R₆₄, —C(═O)OR₆₃, —OC(═O)R₆₄, —C(═O)N(R₃)₂, —NR₆₃C(═O)R₆₄, —OC(═O)OR₆₃, —NR₆₃C(═O)OR₆₃, —OC(═O)N(R₆₃)₂, —NR₆₃C(═O)N(R₆₃)₂, —S(═O)OR₆₃, —OS(═O)R₆₄, —S(═O)N(R₆₃)₂, —NR₆₃S(═O)R₆₄, —S(═O)₂OR₆₃, —OS(═O)₂R₆₄, —S(═O)₂N(R₆₃)₂, —NR₆₃S(═O)₂R₆₄, —OS(═O)₂OR₆₃, —NR₆₃S(═O)₂OR₆₃, —OS(═O)₂N(R₆₃)₂, —NR₆₃S(═O)₂N(R₆₃)₂, —P(R₆₃)₂, —P(═O)(R₆₄)₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;
Optionally, two R_{S1} together with the carbon atom to which they are both attached form a 3-10 membered carbocyclic ring or a 3-10 heterocyclic ring; wherein, said 3-10 membered carbocylic ring or 3-10 heterocyclic ring is optionally substituted with one or more R_{6b};
Optionally, two adjacent R_{S1} together with the carbon atoms to which they are respectively attached form a 3-10 membered carbocyclic ring, a 3-10 membered heterocyclic ring, a 6-10 membered aryl ring or a 5-10 membered heteroaryl ring, wherein, each of rings is independently optionally substituted with one or more R₆ₑ;
Each of q₁ is independently selected from 0, 1, 2, 3, 4, 5 or 6;
L₁ is a bond, O, S, S(═O), S(═O)₂ or NR₆ₐ;
R₁ is selected from or

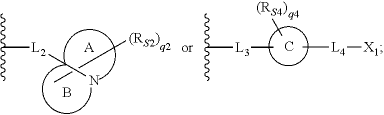

L₂ is selected from a bond or C₁₋₁₀ alkylene optionally substituted with one or more R_{6d};
L₃ is selected from a bond or C₁₋₁₀ alkylene optionally substituted with one or more R₆ₑ;
L₄ is selected from a bond or C₁₋₁₀ alkylene optionally substituted with one or more R_{6f};
Ring A or ring B is independently selected from a 3-10 membered heterocyclic ring which is optionally further contains 1, 2, or 3 heteroatoms selected from N, O or S;
Ring C is selected from a 3-10 membered carbocyclic ring or a 3-10 membered heterocyclic ring; wherein the moiety of -L₃- and -L₄-X₁ are attached to the same atom or different atoms of the ring C;
X₁ is selected from —N(R₆₅)₂, —OR₆₅, —SR₆₅, 3-10 membered heterocyclyl, or 5-10 membered heteroaryl, wherein said 3-10 membered heterocyclyl or 5-10 membered heteroaryl is optionally independently substituted with one or more R_{S3};
Each of (R_{S2} and R_{S3}) at each occurrence is independently selected from halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, oxo, —N(R₆₆)₂, —OR₆₆, —SR₆₆, —S(═O)R₆₇, —S(═O)₂R₆₇, —C(═O)R₆₇, —C(═O)OR₆₆, —OC(═O)R₆₇, —C(═O)N(R₆₆)₂, —NR₆₆C(═O)R₆₇, —OC(═O)OR₆₆, —NR₆₆C(═O)OR₆₆, —NR₆₆C(═S)OR₆₆, —OC(═O)N(R₆₆)₂, —NR₆₆C(═O)N(R₆₆)₂, —S(═O)OR₆₆, —OS(═O)R₆₇, —S(═O)N(R₆₆)₂, —NR₆₆S(═O)R₆₇, —S(═O)₂OR₆₆, —OS(═O)₂R₆₇, —S(═O)₂N(R₆₆)₂, —NR₆₆S(═O)₂R₆₇, —OS(═O)₂OR₆₆, —NR₆₆S(═O)₂OR₆₆, —OS(═O)₂N(R₆₆)₂, —NR₆₆S(═O)₂N(R₆₆)₂, —P(R₆₆)₂, —P(═O)(R₆₇)₂, 3-8 membered cycloalkyl, 3-8 membered cycloalkenyl, 3-8 membered cycloalkynyl, 4-8 membered heterocyclyl, 6-10 membered aryl, 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-8 membered cycloalkyl, 3-8 membered cycloalkenyl, 3-8 membered cycloalkynyl, 3-8 membered heterocyclyl, 6-10 membered aryl or 5-10 membered heteroaryl is optionally independently substituted with one or more substituents selected from halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —N(R₆₈)₂, —OR₆₈, —SR₆₈, —S(═O)R₆₉, —S(═O)₂R₆₉, —C(═O)R₆₉, —C(═O)OR₆₈, —OC(═O)R₆₉, —C(═O)N(R₆₈)₂, —NR₆₈C(═O)R₆₉, —OC(═O)OR₆₈, —NR₆₈C(═O)OR₆₈, —NR₆₈C(═S)OR₆₈, —OC(═O)N(R₆₈)₂, —NR₆₈C(═O)N(R₆₈)₂, —S(═O)OR₆₈, —OS(═O)R₆₉, —S(═O)N(R₆₈)₂, —NR₆₈S(═O)R₆₉, —S(═O)₂OR₆₈, —OS(═O)₂R₆₉, —S(═O)₂N(R₆₈)₂, —NR₆₈S(═O)₂R₆₉, —OS(═O)₂OR₆₈, —NR₆₈S(═O)₂OR₆₈, —OS(═O)₂N(R₆₈)₂, —NR₆₈S(═O)₂N(R₆₈)₂, —P(R₆₈)₂, —P(═O)(R₆₉)₂, 3-6 membered cycloalkyl, 3-6 membered cycloalkenyl, 3-6 membered cycloalkynyl, 3-6 membered heterocyclyl, 6-10 membered aryl or 5-10 membered heteroaryl;
Optionally, two R_{S2} together with the carbon atom to which they are both attached or two R_{S3} together with the carbon atom to which they are both attached form a 3-10 membered carbocyclic ring or a 3-10 heterocyclic ring; wherein, sais 3-10 membered carbocylic ring or 3-10 heterocyclic ring is optionally substituted with one or more R₆ₕ;
Optionally, two adjacent R_{S2} together with the carbon atoms to which they are respectively attached or two adjacent R_{S3} together with the carbon atoms to which they are respectively attached form a 3-10 membered carbocyclic ring, a 3-10 membered heterocyclic ring, a 6-10 membered aryl ring or a 5-10 membered heteroaryl ring, wherein, each of rings is independently optionally substituted with one or more R₆ᵢ;
Optionally, two nonadjacent R_{S2} or two nonadjacent R_{S3} are connected together to form a bridge containing 0, 1, 2, 3, 4, 5 or 6 carbon atoms, wherein, each of the carbon atoms in the bridge is optionally replaced by 1 or 2 heteroatoms selected from N, O, S, S═O or S(═O)₂; the hydrogen on the each of carbon atoms or N atoms is optionally independently substituted with R₆ⱼ;
q₂ is selected from 0, 1, 2, 3, 4, 5 or 6;
Each of R_{S4} at each occurrence is independently selected from halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, oxo, —N(R₇₁)₂, —OR₇₁, —SR₇₁, —S(═O)R₇₂, —S(═O)₂R₇₁, —C(═O)R₇₂, —C(═O)OR₇₁, —OC(═O)R₇₂, —C(═O)N(R₇₁)₂, —NR₇₁C(═O)R₇₂, —OC(═O)OR₇₁, —NR₇₁C(═O)OR₇₁, —OC(═O)N(R₇₁)₂, —NR₇₁C(═O)N(R₇₁)₂, —S(═O)OR₇₁, —OS(═O)R₇₂, —S(═O)N(R₇₁)₂, —NR₇₁S(═O)R₇₂, —S(═O)₂OR₇₁, —OS(═O)₂R₇₂, —S(═O)₂N(R₇₁)₂, —NR₇₁S(═O)₂R₇₂, —OS(═O)₂OR₇₁, —NR₇₁S(═O)₂OR₇₂, —OS(═O)₂N(R₇₁)₂, —NR₇₁S(═O)₂N(R₇₁)₂, —P(R₇₁)₂, —P(═O)(R₇₂)₂, 3-6 membered cycloalkyl, 3-6 membered cycloalkenyl, 3-6 membered cycloalkynyl, 3-6 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-6 membered cycloalkyl, 3-6 membered cycloalkenyl, 3-6 membered cycloalkynyl, 3-6 membered heterocyclyl, 6-10 membered aryl or 5-10 membered heteroaryl is optionally independently substituted with one or more substituents selected from halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —N(R₇₃)₂, —OR₇₃, —SR₇₃, —S(═O)R₇₄, —S(═O)₂R₇₃, —C(═O)R₇₄, —C(═O)OR₇₃, —OC(═O)R₇₄, —C(═O)N(R₇₃)₂, —NR₇₃C(═O)R₇₄, —OC(═O)OR₇₃, —NR₇₃C(═O)OR₇₃, —OC(═O)N(R₇₃)₂, —NR₇₃C(═O)N(R₇₃)₂, —S(═O)OR₇₃, —OS(═O)R₇₄, —S(═O)N(R₇₃)₂, —NR₇₃S(═O)R₇₄, —S(═O)₂OR₇₃, —OS(═O)₂R₇₄, —S(═O)₂N(R₇₃)₂, —NR₇₃S(═O)₂R₇₄, —OS(═O)₂OR₇₃, —NR₇₃S(═O)₂OR₇₄, —OS(═O)₂N(R₇₃)₂, —NR₇₃S(═O)₂N(R₇₃)₂, —P(R₇₃)₂, —P(═O)(R₇₄)₂, 3-6 membered cycloalkyl, 3-6 membered cycloalkenyl, 3-6 membered cycloalkynyl, 3-6 membered heterocyclyl, 6-10 membered aryl or 5-10 membered heteroaryl;
q₄ is selected from 0, 1, 2, 3, 4, 5 or 6;
R₃ is selected from 6-10 membered aryl or 5-10 membered heteroaryl, wherein said 6-10 membered aryl or 5-10 membered heteroaryl is optionally independently substituted with one or more R₃₁;
R₃₁ at each occurrence is independently selected from halogen, —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, —CN, oxo, —N(R₇₅)₂, —OR₇₅, —SR₇₅, —S(═O)R₇₆, —S(═O)₂R₇₆, —C(═O)R₇₆, —C(═O)OR₇₅, —OC(═O)R₇₆, —C(═O)N(R₇₅)₂, —NR₇₅C(═O)R₇₆, —OC(═O)OR₇₅, —NR₇₅C(═O)OR₇₅, —OC(═O)N(R₇₅)₂, —NR₇₅C(═O)N(R₇₅)₂, —S(═O)OR₇₅, —OS(═O)R₇₆, —S(═O)N(R₇₅)₂, —NR₇₅S(═O)R₇₆, —S(═O)₂OR₇₅, —OS(═O)₂R₇₆, —S(═O)₂N(R₇₅)₂, —NR₇₅S(═O)₂R₇₆, —OS(═O)₂OR₇₅, —NR₇₅S(═O)₂OR₇₅, —OS(═O)₂N(R₇₅)₂, —NR₇₅S(═O)₂N(R₇₅)₂, —P(R₇₅)₂, —P(═O)(R₇₅)₂, 3-10 membered cycloalkyl, 3-10 membered cycloalkenyl, 3-10 membered cycloalkynyl, 3-10 membered heterocyclyl, 6-10 membered aryl or 5-10 membered heteroaryl, wherein said —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is optionally independently substituted with one or more substituents selected from halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —N(R₇₇)₂, —OR₇₇, —SR₇₇, —S(═O)R₇₈, —S(═O)₂R₇₈, —C(═O)R₇₈, —C(═O)OR₇₇, —OC(═O)R₇₈, —C(═O)N(R₇₇)₂, —NR₇₇C(═O)R₇₈, —OC(═O)OR₇₇, —NR₇₇C(═O)OR₇₇, —OC(═O)N(R₇₇)₂, —NR₇₇C(═O)N(R₇₇)₂, —S(═O)OR₇₇, —OS(═O)R₇₈, —S(═O)N(R₇₇)₂, —NR₇₇S(═O)R₇₈, —S(═O)₂OR₇₇, —OS(═O)₂R₇₈, —S(═O)₂N(R₇₇)₂, —NR₇₇S(═O)₂R₇₈, —OS(═O)₂OR₇₇, —NR₇₇S(═O)₂OR₇₇, —OS(═O)₂N(R₇₇)₂, —NR₇₇S(═O)₂N(R₇₇)₂, —P(R₇₇)₂, —P(═O)(R₇₈)₂, 3-6 membered cycloalkyl, 3-6 membered cycloalkenyl, 3-6 membered cycloalkynyl, 3-6 membered heterocyclyl, 6-10 membered aryl or 5-10 membered heteroaryl;

Each of (R₂, R₄ and R₅) is independently selected from hydrogen, halogen, —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, —CN, oxo, —N(R₈₁)₂, —OR₈₁, —SR₈₁, —S(═O)R₈₂, —S(═O)₂R₈₂, —C(═O)R₈₂, —C(═O)OR₈₁, —OC(═O)R₈₂, —C(═O)N(R₈₁)₂, —NR₈₁C(═O)R₈₂, —OC(═O)OR₈₁, —NR₈₁C(═O)OR₈₁, —OC(═O)N(R₈₁)₂, —NR₈₁C(═O)N(R₈₁)₂, —S(═O)OR₈₁, —OS(═O)R₈₂, —S(═O)N(R₈₁)₂, —NR₈₁S(═O)R₈₂, —S(═O)₂OR₈₁, —OS(═O)₂R₈₂, —S(═O)₂N(R₈₁)₂, —NR₈₁S(═O)₂R₈₂, —OS(═O)₂OR₈₁, —NR₈₁S(═O)₂OR₈₁, —OS(═O)₂N(R₈₁)₂, —NR₈₁S(═O)₂N(R₈₁)₂, —P(R₈₁)₂, —P(═O)(R₈₂)₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is optionally independently substituted with one or more substituents selected from halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —N(R₈₃)₂, —OR₈₃, —SR₈₃, —S(═O)R₈₄, —S(═O)₂R₈₄, —C(═O)R₈₄, —C(═O)OR₈₃, —OC(═O)R₈₃, —C(═O)N(R₈₃)₂, —NR₈₃C(═O)R₈₄, —OC(═O)OR₈₃, —NR₈₃C(═O)OR₈₃, —OC(═O)N(R₈₃)₂, —NR₈₃C(═O)N(R₈₄)₂, —S(═O)OR₈₃, —OS(═O)R₈₄, —S(═O)N(R₈₃)₂, —NR₈₃S(═O)R₈₄, —S(═O)₂OR₈₃, —OS(═O)₂R₈₄, —S(═O)₂N(R₈₃)₂, —NR₈₃S(═O)₂R₈₄, —OS(═O)₂OR₈₃, —NR₈₃S(═O)₂OR₈₃, —OS(═O)₂N(R %₃)₂, —NR₈₃S(═O)₂N(R₈₃)₂, —P(R₈₃)₂, —P(═O)(R₈₄)₂, 3-6 membered cycloalkyl, 3-6 membered cycloalkenyl, 3-6 membered cycloalkynyl, 3-6 membered heterocyclyl, 6-10 membered aryl or 5-10 membered heteroaryl;

Each of (R₆ₐ, R₆₁, R₆₃, R₆₅, R₆₆, R₆₈, R₇₁, R₇₃, R₇₅, R₇₇, R₈₁ and R₈₃) at each occurrence is independently selected from hydrogen, halogen, —C₁₋₁₀alkyl, haloC₁₋₁₀alkyl, —C₂₋₁₀alkenyl, —C₂₋₁₀alkynyl, —S(═O)Rₐ, —S(═O)₂Rₐ, —C(═O)Rₐ, —C(═O)ORₐ, —C(═O)N(Rₐ)₂, —S(═O)ORₐ, —S(═O)N(Rₐ)₂, —S(O)₂ORₐ, —S(═O)₂N(Rₐ)₂, —P(═O)(Rₐ)₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₁₀alkyl, haloC₁₋₁₀alkyl, —C₂₋₁₀alkenyl, —C₂₋₁₀alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is optionally independently substituted with one or more substituents selected from halogen, —C₁₋₆alkyl, haloC₁₋₆alkyl, haloC₁₋₆alkoxy, —C₂₋₆alkenyl, —C₂₋₆alkynyl, —CN, —N(R_{c})₂, —OR_{c}, —SR_{c}, —S(═O)R_{d}, —S(═O)₂R_{d}, —C(═O)R_{d}, —C(═O)OR_{c}, —OC(═O)R_{d}, —C(═O)N(R_{c})₂, —NR_{c}C(═O)R_{d}, —OC(═O)OR_{c}, —NR_{c}C(═O)OR_{d}, —OC(═O)N(R_{c})₂, —NR_{c}C(═O)N(R_{c})₂, —S(═O)OR_{c}, —OS(═O)R_{d}, —S(═O)N(R_{c})₂, —NR_{c}S(═O)R_{d}, —S(═O)₂OR_{c}, —OS(═O)₂R_{d}, —S(═O)₂N(R_{c})₂, —NR_{c}S(═O)₂R_{d}, —OS(═O)₂OR_{c}, —NR_{c}S(═O)₂OR_{c}, —OS(═O)₂NR_{c}, —NR_{c}S(═O)₂N(R_{c})₂, —P(R_{c})₂, —P(═O)(R_{d})₂, 3-6 membered cycloalkyl, 3-6 membered cycloalkenyl, 3-6 membered cycloalkynyl, 3-6 membered heterocyclyl, 6-10 membered aryl or 5-10 membered heteroaryl;

Optionally, each of (two R₆₁, two R₆₃, two R₆₅, two R₆₆, two R₆₈, two R₇₁, two R₇₃, two R₇₅, two R₇₇, two R₈₁, and two R₈₃) independently together with the nitrogen atom to which they are both attached forms a 3-20 membered heterocyclic ring or a 5-10 membered heteroaryl ring, wherein, said 3-20 membered heterocyclic ring or 5-10 membered heteroaryl ring is optionally independently substituted with one or more R₆ₖ;

Each of (R₆₂, R₆₄, R₆₇, R₆₉, R₇₂, R₇₄, R₇₆, R₇₈, R₈₂ and R₈₄) at each occurrence is independently selected from hydrogen, —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, —N(R_{b})₂, —OR_{b}, —SR_{b}, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₁₀alkyl, haloC₁₋₁₀alkyl, haloC₁₋₁₀alkoxy, —C₂₋₁₀alkenyl, —C₂₋₁₀alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is optionally independently substituted with one or more substituents selected from halogen, —C₁₋₆alkyl, haloC₁₋₆alkyl, haloC₁₋₆alkoxy, —C₂₋₆alkenyl, —C₂₋₆alkynyl, —CN, —N(R_{c})₂, —OR_{c}, —SR_{c}, —S(═O)R_{d}, —S(═O)₂R_{d}, —C(═O)R_{d}, —C(═O)OR_{c}, —OC(═O)R_{d}, —C(═O)N(R_{c})₂, —NR_{c}C(═O)R_{d}, —OC(═O)OR_{c}, —NR_{c}C(═O)OR_{d}, —OC(═O)N(R_{c})₂, —NR_{c}C(═O)N(R_{c})₂, —S(═O)OR_{c}, —OS(═O)R_{d}, —S(═O)N(R_{c})₂, —NR_{c}S(═O)R_{d}, —S(═O)₂OR_{c}, —OS(═O)₂R_{d}, —S(═O)₂N(R_{c})₂, —NR_{c}S(═O)₂R_{d}, —OS(═O)₂OR_{c}, —NR_{c}S(═O)₂OR_{c}, —OS(═O)₂NR_{c}, —NR_{c}S(═O)₂N(R_{c})₂, —P(R_{c})₂, —P(═O)(R_{d})₂, 3-6 membered cycloalkyl, 3-6 membered cycloalkenyl, 3-6 membered cycloalkynyl, 3-10 membered heterocyclyl, 6-10 membered aryl or 5-10 membered heteroaryl;

Each of (Rₐ, R_{b}, R_{c} and R_{d}) at each occurrence is independently selected from hydrogen, —C₁₋₆alkyl, haloC₁₋₆alkyl, haloC₁₋₆alkoxy, —C₂₋₆alkenyl, —C₂₋₆alkynyl, 3-6 membered cycloalkyl, 3-6 membered cycloalkenyl, 3-6 membered cycloalkynyl, 3-6 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, —C₂₋₆alkynyl, 3-6 membered cycloalkyl, 3-6 membered cycloalkenyl, 3-6 membered cycloalkynyl, 3-6 membered heterocyclyl, 6-10 membered aryl or 5-10 membered heteroaryl is optionally independently substituted with one or more R₆ₗ;

Optionally, each of (two Rₐ, two R_{b} and two R_{c}) independently together with the atom to which they are both attached forms a 3-6 membered heterocyclic ring, wherein said 3-6 membered heterocyclic ring is independently optionally substituted with one or more R₆ₘ;

Each of (R_{6b}, R_{6c}, R_{6d}, R₆ₑ, R_{6f}, R₆ₕ, R₆ᵢ, R₆ⱼ, R₆ₖ, R₆ₗ and R₆ₘ) at each occurrence is independently selected from halogen, —C₁₋₆alkyl, haloC₁₋₆alkyl, haloC₁₋₆alkoxy, —C₂₋₆alkenyl, —C₂₋₆alkynyl, —CN, oxo, —NH₂, —NH(C₁₋₆alkyl), —N(C₁₋₆alkyl)₂, —OH, —O(C₁₋₆alkyl), —SH, —S(C₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₆alkyl)₂, —NHC(═O)(C₁₋₆alkyl), —N(C₁₋₆alkyl)C(═O)(C₁₋₆alkyl), —OC(═O)O(C₁₋₆alkyl), —NHC(═O)(OC₁₋₆alkyl), —N(C₁₋₆alkyl)C(═O)(OC₁₋₆alkyl), —OC(═O)NH(C₁₋₆alkyl), —OC(═O)N(C₆alkyl)₂, —NHC(═O)NH₂, —NHC(═O)NH(C₁₋₆alkyl), —NHC(═O)N(C₁₋₆alkyl)₂, —N(C₁₋₆alkyl)C(═O)NH₂, —N(C₁₋₆alkyl)C(═O)NH(C₁₋₆alkyl), —N(C₁₋₆alkyl)C(═O)N(C₁₋₆alkyl)₂, —S(═O)(OC₁₋₆alkyl), —OS(═O)(C₁₋₆alkyl), —S(═O)NH₂, —S(═O)NH(C₁₋₆alkyl), —S(═O)N(C₆alkyl)₂, —NHS(═O)(C₁₋₆alkyl), —N(C₁₋₆alkyl)S(═O)(C₁₋₆alkyl), —S(═O)₂(OC₁₋₆alkyl), —OS(O)₂(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), —OS(═O)₂O(C₁₋₆alkyl), —NHS(═O)₂O(C₆alkyl), —N(C₁₋₆alkyl)S(═O)₂O(C₁₋₆alkyl), —OS(═O)₂NH₂, —OS(═O)₂NH(C₁₋₆alkyl), —OS(═O)₂N(C₆alkyl)₂, —NHS(═O)₂NH₂, —NHS(═O)₂NH(C₁₋₆alkyl), —NHS(═O)₂N(C₁₋₆alkyl)₂, —N(C₁₋₆alkyl)S(═O)₂NH₂, —N(C₁₋₆alkyl)S(═O)₂NH(C₁₋₆alkyl), —N(C₁₋₆alkyl)S(═O)₂N(C₁₋₆alkyl)₂, —PH(C₁₋₆alkyl), —P(C₁₋₆alkyl)₂, —P(═O)H(C₁₋₆alkyl), —P(═O)(C₁₋₆alkyl)₂, 3-6 membered cycloalkyl, 3-6 membered cycloalkenyl, 3-6 membered cycloalkynyl, 3-6 membered heterocyclyl, 6-10 membered aryl or 5-10 membered heteroaryl; wherein, said —C₁₋₆alkyl, haloC₁₋₆alkyl, haloC₁₋₆alkoxy, —C₂₋₆alkenyl, —C₂₋₆alkynyl, 3-6 membered cycloalkyl, 3-6 membered cycloalkenyl, 3-6 membered cycloalkynyl, 3-6 membered heterocyclyl, 6-10 membered aryl or 5-10 membered heteroaryl is optionally substituted with one or more substituents selected from halogen, —C₁₋₃alkyl, haloC₁₋₃alkyl, haloC₁₋₃alkoxy, —C₂₋₃alkenyl, —C₂₋₃alkynyl, —CN, —NH₂, —NH(C₁₋₃alkyl), —N(C₁₋₃alkyl)₂, —OH, —O(C₁₋₃alkyl), —SH, —S(C₁₋₃alkyl), —S(═O)(C₁₋₃alkyl), —S(═O)₂(C₁₋₃alkyl), —C(═O)(C₁₋₃alkyl), —C(═O)OH, —C(═O)(OC₁₋₃alkyl), —OC(═O)(C₁₋₃alkyl), —C(═O)NH₂, —C(═O)NH(C₁₋₃alkyl), —C(═O)N(C₁₋₃alkyl)₂, —NHC(═O)(C₁₋₃alkyl), —N(C₁₋₃alkyl)C(═O)(C₁₋₃alkyl), —OC(═O)O(C₁₋₃alkyl), —NHC(═O)(OC₁₋₃alkyl), —N(C₁₋₃alkyl)C(═O)(OC₁₋₃alkyl), —OC(═O)NH(C₁₋₃alkyl), —OC(═O)N(C₁₋₃alkyl)₂, —NHC(═O)NH₂, —NHC(═O)NH(C₁₋₃alkyl), —NHC(═O)N(C₁₋₃alkyl)₂, —N(C₁₋₃alkyl)C(═O)NH₂, —N(C₁₋₃alkyl)C(═O)NH(C₁₋₃alkyl), —N(C₁₋₃alkyl)C(═O)N(C₁₋₃alkyl)₂, —S(═O)(OC₁₋₃alkyl), —OS(═O)(C₁₋₃alkyl), —S(═O)NH₂, —S(═O)NH(C₁₋₃alkyl), —S(═O)N(C₁₋₃alkyl)₂, —NHS(═O)(C₁₋₃alkyl), —N(C₁₋₃alkyl)S(═O)(C₁₋₃alkyl), —S(═O)₂(OC₁₋₃alkyl), —OS(═O)₂(C₁₋₃alkyl), —S(═O)₂NH₂, —S(═O)₂NH(C₁₋₃alkyl), —S(═O)₂N(C₁₋₃alkyl)₂, —NHS(═O)₂(C₁₋₃alkyl), —N(C₁₋₃alkyl)S(═O)₂(C₁₋₃alkyl), —OS(═O)₂O(C₁₋₃alkyl), —NHS(═O)₂O(C₁₋₃alkyl), —N(C₁₋₃alkyl)S(═O)₂O(C₁₋₃alkyl), —OS(═O)₂NH₂, —OS(═O)₂NH(C₁₋₃alkyl), —OS(═O)₂N(C₁₋₃alkyl)₂, —NHS(═O)₂NH₂, —NHS(═O)₂NH(C₁₋₃alkyl), —NHS(═O)₂N(C₁₋₃alkyl)₂, —N(C₁₋₃alkyl)S(═O)₂NH₂, —N(C₁₋₃alkyl)S(═O)₂NH(C₁₋₃alkyl), —N(C₁₋₃alkyl)S(═O)₂N(C₁₋₃alkyl)₂, —PH(C₁₋₃alkyl), —P(C₁₋₃alkyl)₂, —P(═O)H(C₁₋₃alkyl), —P(═O)(C₁₋₃alkyl)₂, 3-6 membered cycloalkyl, 3-6 membered cycloalkenyl, 3-6 membered cycloalkynyl, 3-6 membered heterocyclyl, 6 membered aryl or 5-6 membered heteroaryl;

Each of (heterocyclyl and heteroaryl) at each occurrence is independently contain 1, 2, 3 or 4 heteroatoms selected from N, O, S, S(═O) or S(═O)₂.

[2] The compound of [1], wherein,
L₁ is a bond, O, S, S(═O), S(═O)₂ or NR₆ₐ;
R₁ is selected from

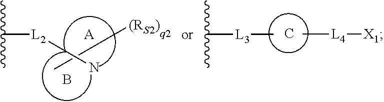

L₂ is selected from a bond or C₁₋₃alkylene optionally substituted with one or more R_{6d};
L₃ is selected from a bond or C₁₋₃alkylene optionally substituted with one or more R₆ₑ;
L₄ is selected from a bond or C₁₋₃ alkylene optionally substituted with one or more R_{6f};
Ring A or ring B is a 5 membered heterocyclic ring which is optionally further contains 1, 2, or 3 heteroatoms selected from N, O or S;
Ring C is selected from a 3-6 membered carbocyclic ring or a 3-6 heterocyclic ring; wherein the moiety of -L₃- and -L₄-X₁ are attached to the same atom or different atoms of the ring C;
X₁ is selected from —N(R₆₅)₂, 5-6 membered heterocyclyl, or 5-10 membered heteroaryl, wherein said 5-6 membered heterocyclyl or 5-10 membered heteroaryl is optionally independently substituted with 1, 2, 3, 4, 5, or 6 R_{S3}; and
R_{S2}, R_{S3}, q₂, R_{6d}, R₆ₑ, R_{6f}, and R₆₅ have the same definition as in [1].

[3] The compound of [1] or [2], wherein,
-L₁-R₁ is selected from

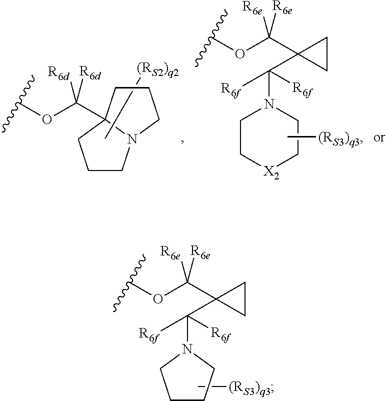

wherein q₃ is selected from 0, 1, 2, 3, 4, 5, or 6.

[4] The compound of any one of [1] to [3], wherein,
the compound is selected from any one of formula (II-1) to (II-6):

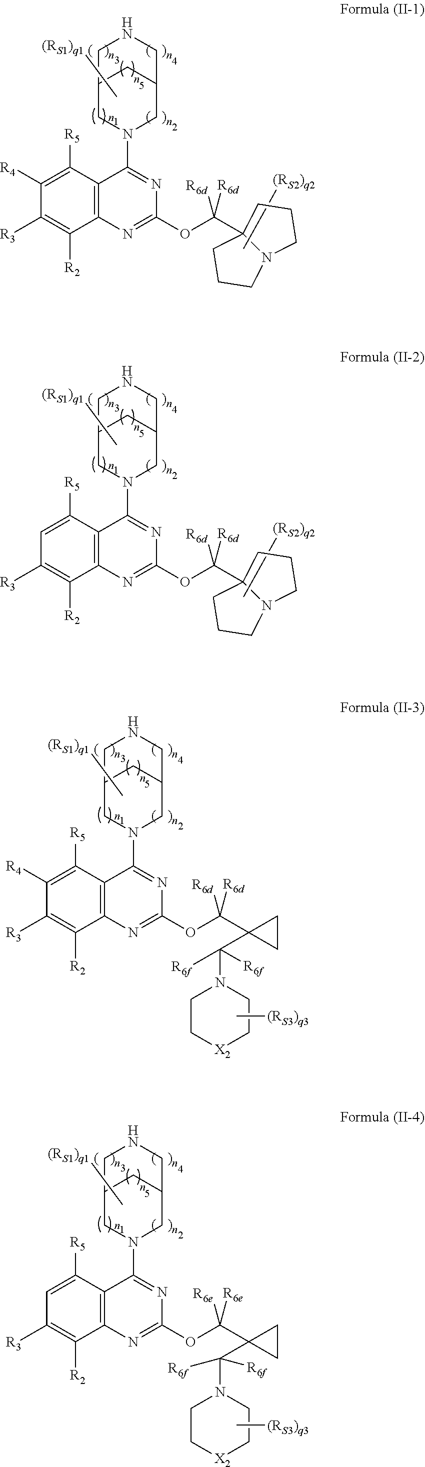

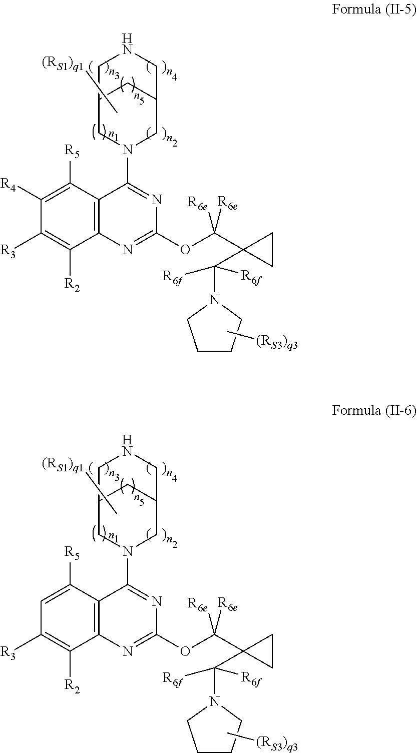

Wherein,
R₄ is selected from halogen, —C₁₋₆alkyl, haloC₁₋₆alkyl, haloC₁₋₆alkoxy, —C₂₋₆alkenyl, —C₂₋₆alkynyl, —CN, —N(R₈₁)₂, —OR₈₁, —SR₈₁, —S(═O)R₈₂, —S(═O)₂R₈₂, —C(═O)R₈₂, —C(═O)OR₈₁, —OC(═O)R₈₂, —P(═O)(R₈₂)₂, 3-10 membered cycloalkyl, or 3-10 membered heterocyclyl, wherein said —C₁₋₆alkyl, haloC₁₋₆alkyl, haloC₁₋₆alkoxy, —C₂₋₆alkenyl, —C₂₋₆alkynyl, 3-10 membered cycloalkyl, or 3-10 membered heterocyclyl is optionally independently substituted with 1, 2, or 3 substituents selected from halogen, —C₁₋₃alkyl, —C₂₋₃alkenyl, —C₂₋₃alkynyl, —CN, —N(R₈₃)₂, —OR₈₃, —SR₈₃, —S(═O)R₈₄, —S(═O)₂R₈₄, —C(═O)R₈₄, —C(═O)OR₈₃, —OC(═O)R₈₃, —P(═O)(R₈₄)₂, 3-6 membered cycloalkyl, or 3-6 membered heterocyclyl;

Each of (R₈₁ and R₈₃) at each occurrence is independently selected from hydrogen, —C₁₋₆alkyl, —C₂₋₆alkenyl, or —C₂₋₆alkynyl, wherein said —C₁₋₆alkyl, —C₂₋₆alkenyl, or —C₂₋₆alkynyl is optionally independently substituted with 1, 2, or 3 substituents selected from halogen;

Each of (R₈₂ and R₈₄) at each occurrence is independently selected from hydrogen, —C₁₋₃alkyl, —C₂₋₃alkenyl, or —C₂₋₃alkynyl, wherein said —C₁₋₆alkyl, —C₂₋₆alkenyl, or —C₂₋₆alkynyl is optionally independently substituted with 1, 2, or 3 substituents selected from halogen;

Preferably, R₄ is selected from

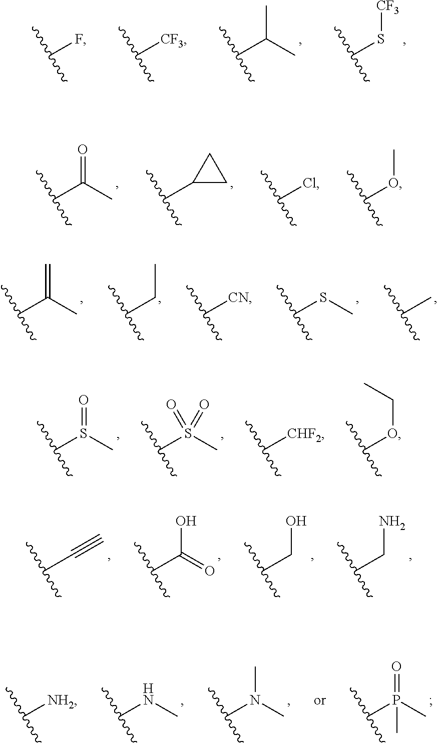

More preferably, R₄ is selected from

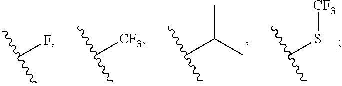

X₂ is selected from CH₂, NH, O, S, S═O, or O═S═O; and
R₂, R₃, R₅, R_{S1}, R_{S2}, R_{S3}, R_{6d}, R₆ₑ, R_{6f}, n₁, n₂, n₃, n₄, n₅, q₁, q₂, and q₃ have the same definitions as in any one of [1] to [3].

[5] The compound of any one of [1] to [4], wherein,
n₃, n₄, n₅ or n₆ is independently selected from 0 or 1, and n₇ is selected from 1 or 2; provided that satisfies one of the following conditions:
n₃ and n₄ are 0, and n₅ and n₆ are 1;
n₃ and n₄ are 1, and n₅ and n₆ are 0;
n₃ and n₅ are 0, and n₄ and n₆ are 1; or
n₃ and n₅ are 1, and n₄ and n₆ are 0.

[6] The compound of any one of [1] to [5], wherein,
the moiety of

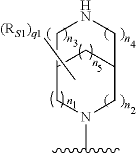

is selected from

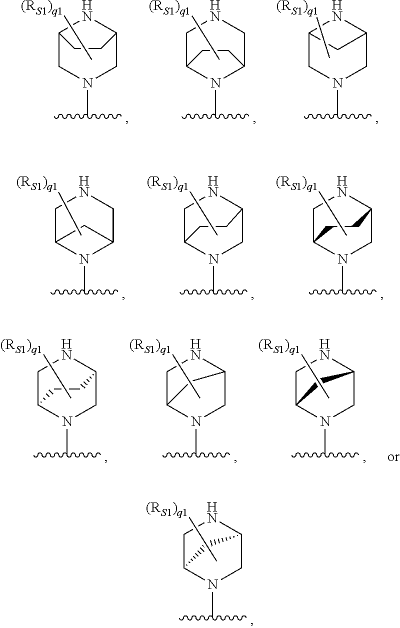

preferably, the moiety of

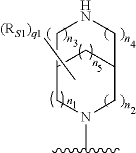

is selected from

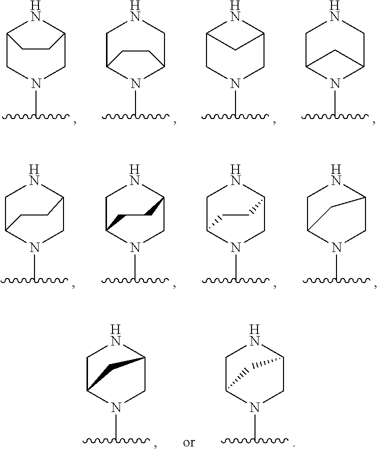

[7] The compound of any one of [1] to [6], wherein,
the compound is selected from any one of formula (III-1) to (III-60):

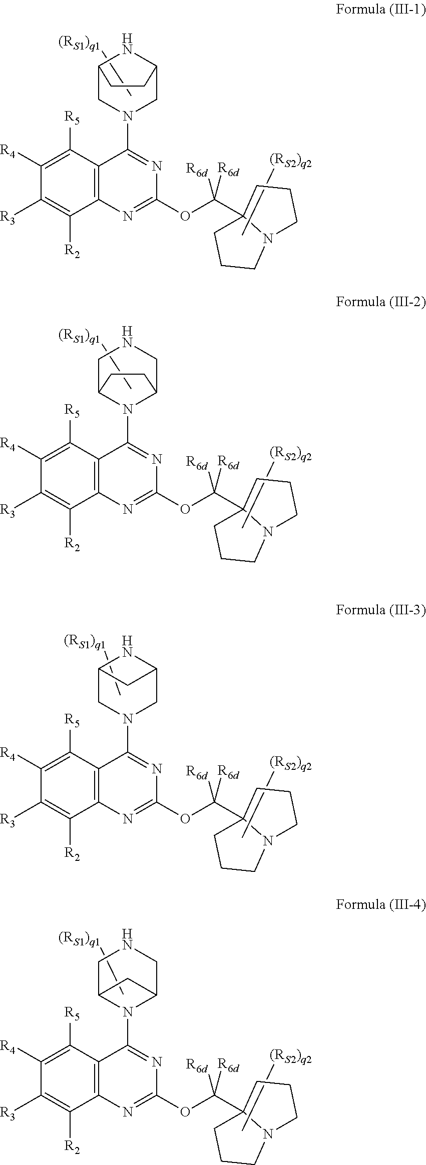

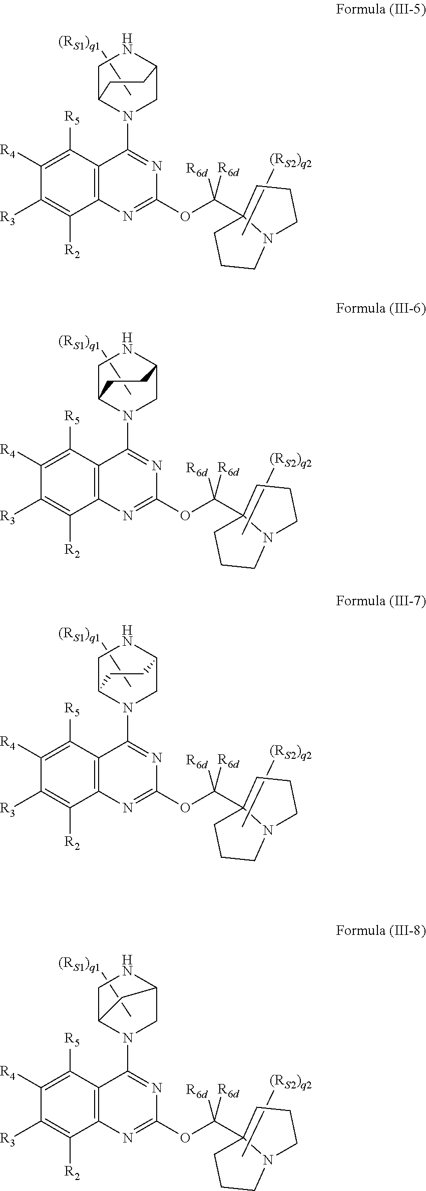

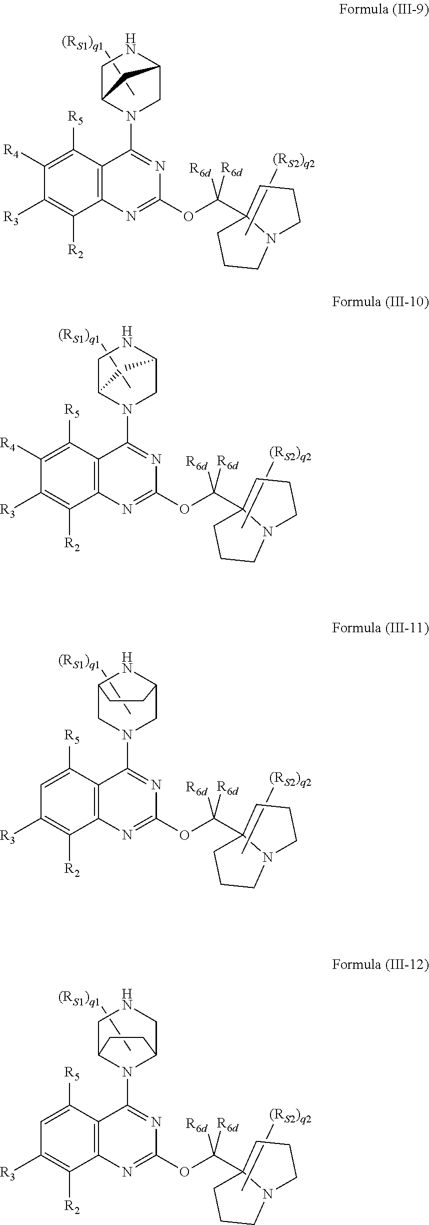

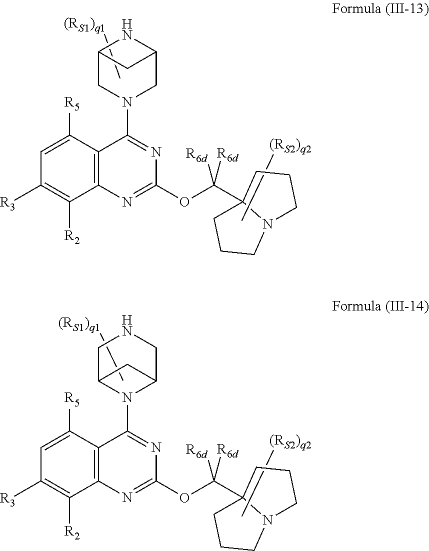

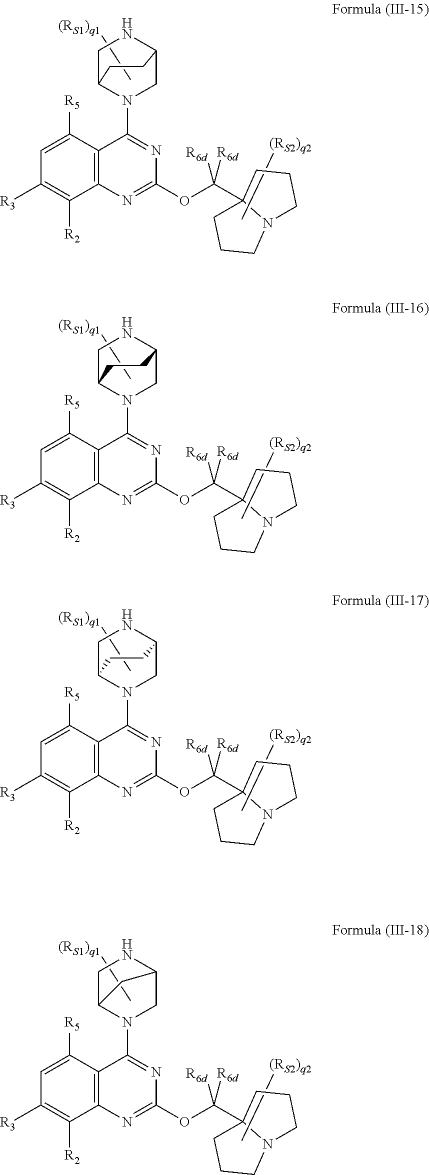

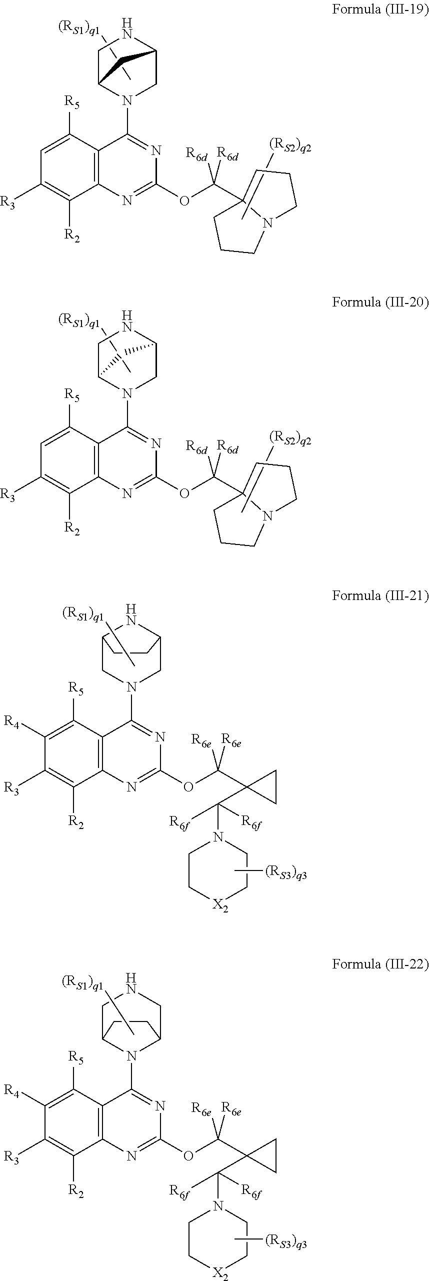

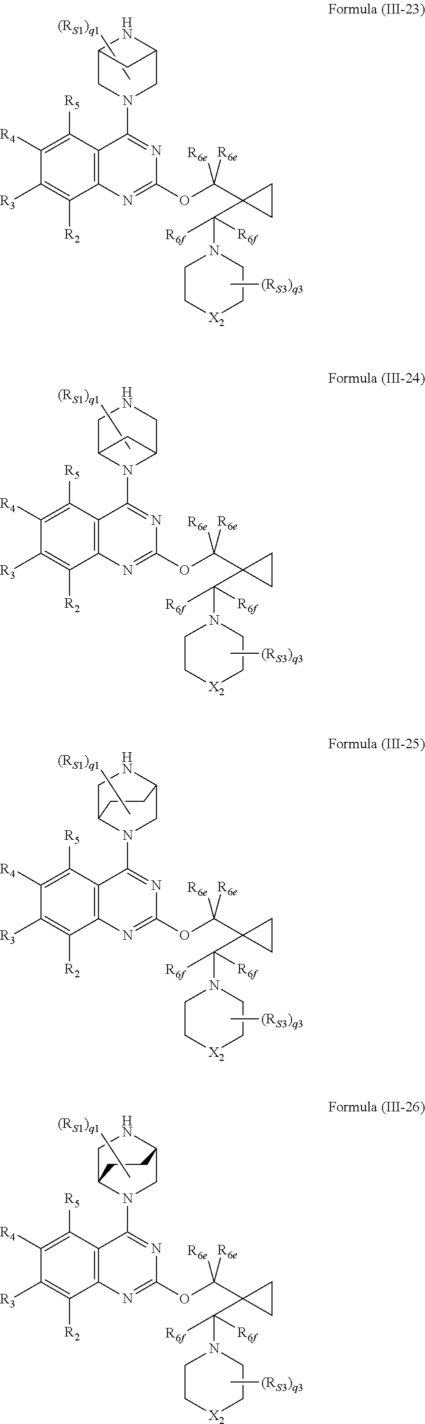

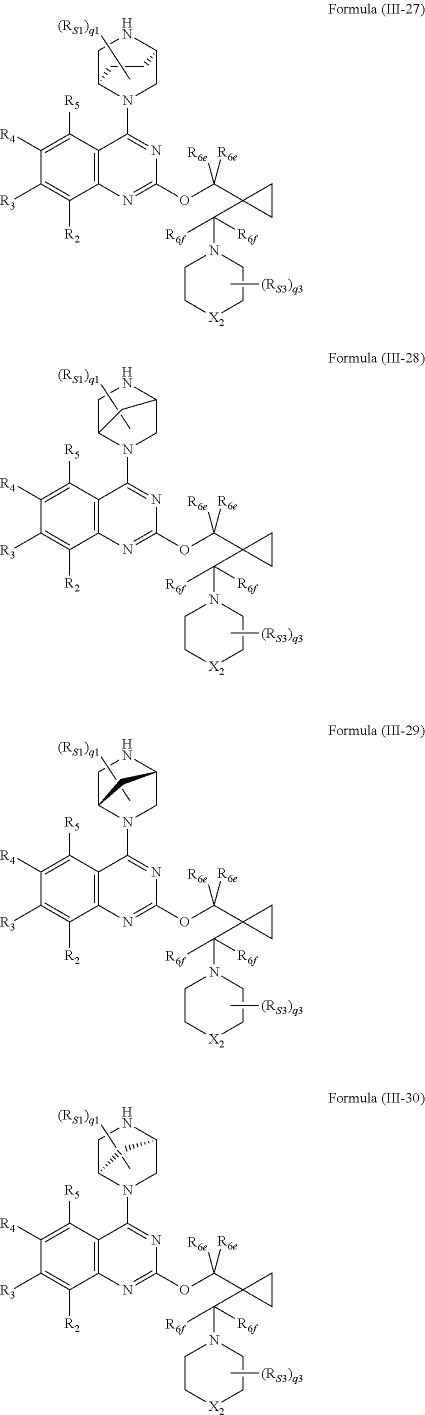

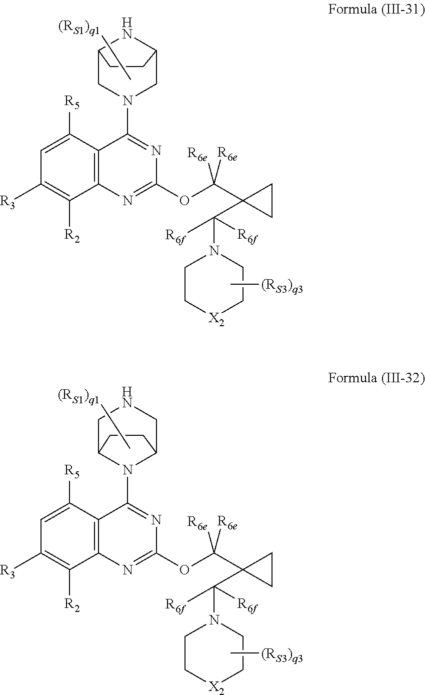

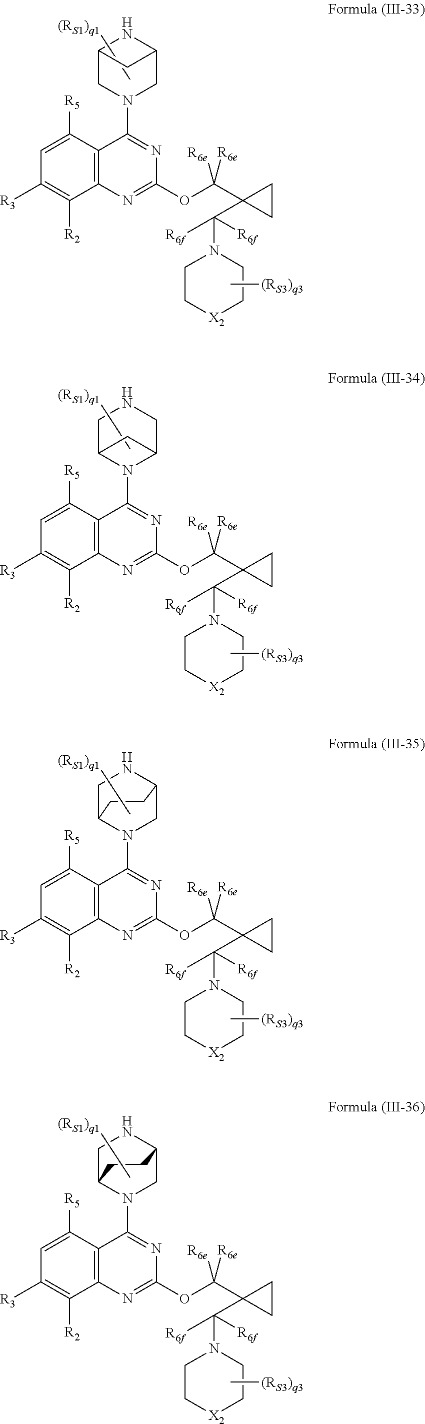

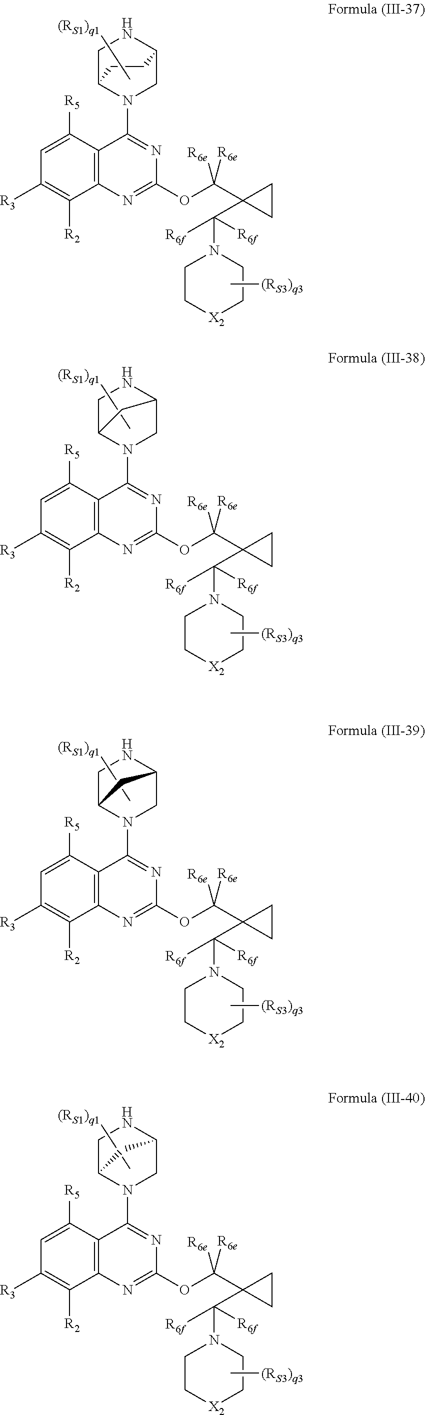

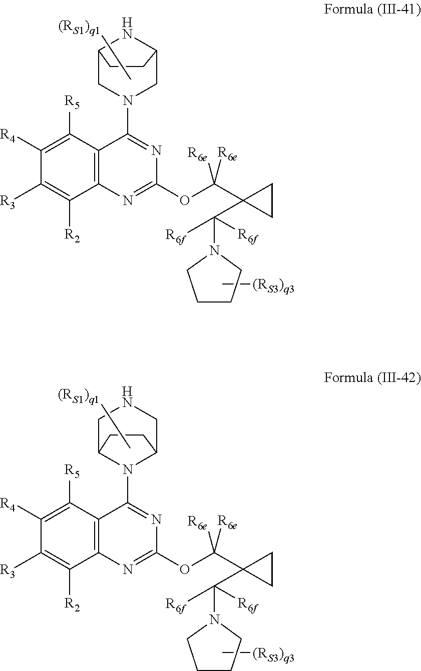

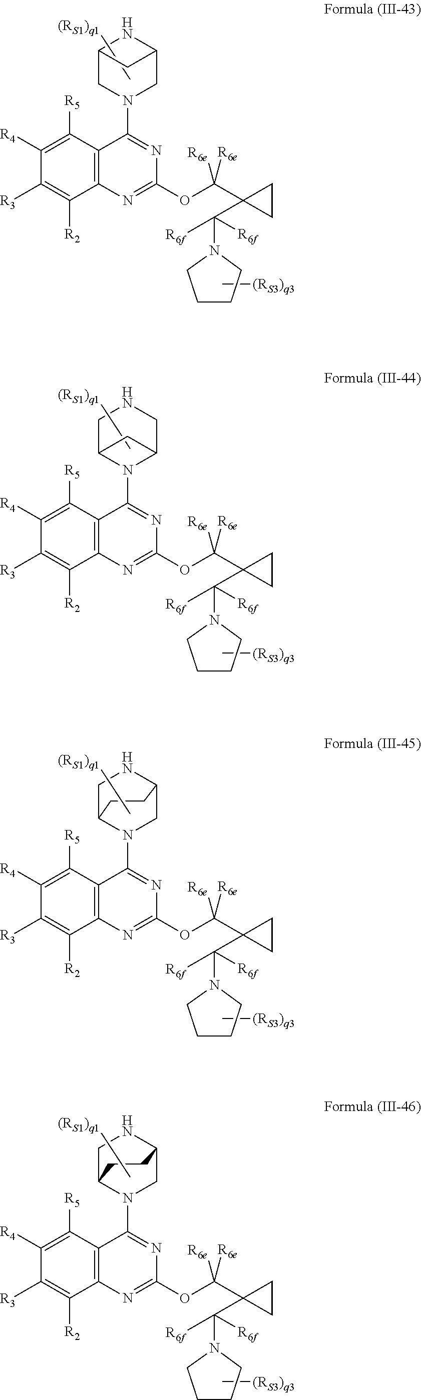

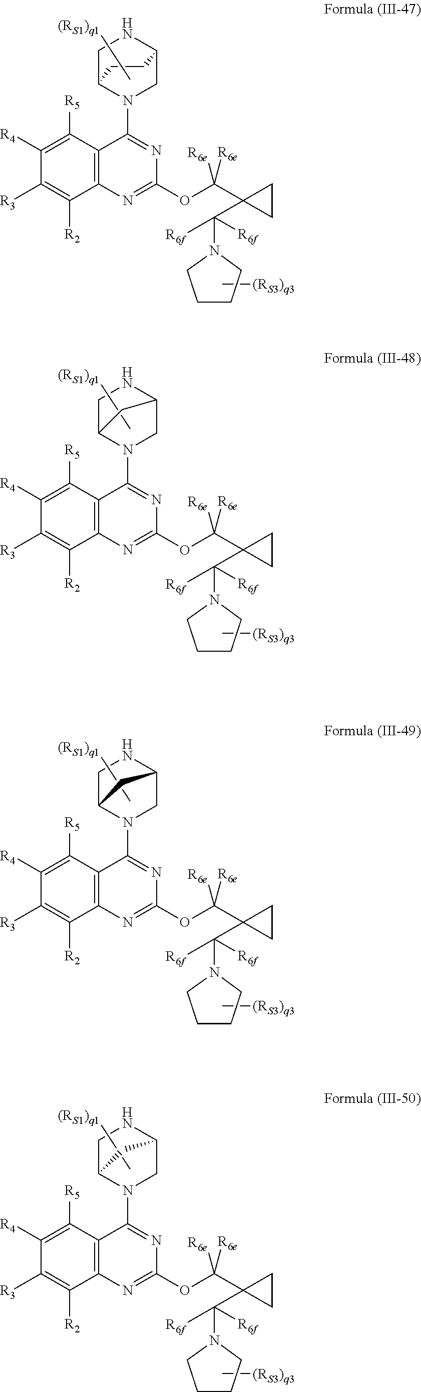

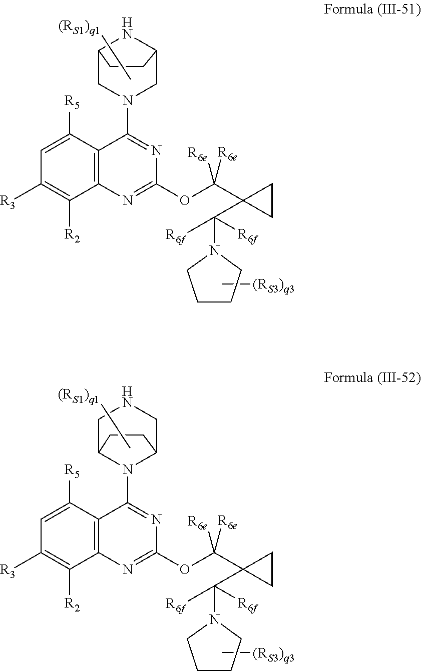

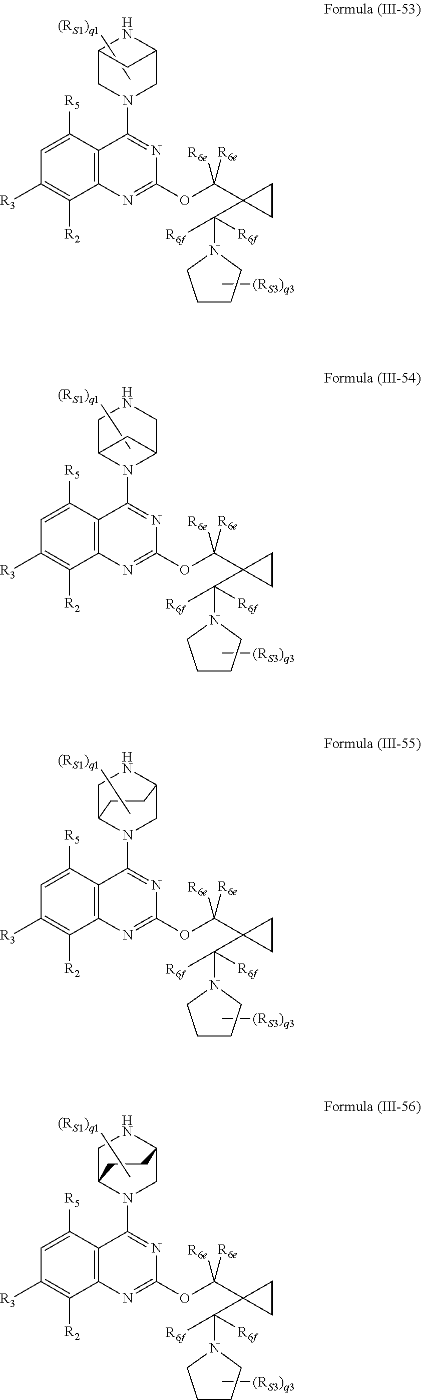

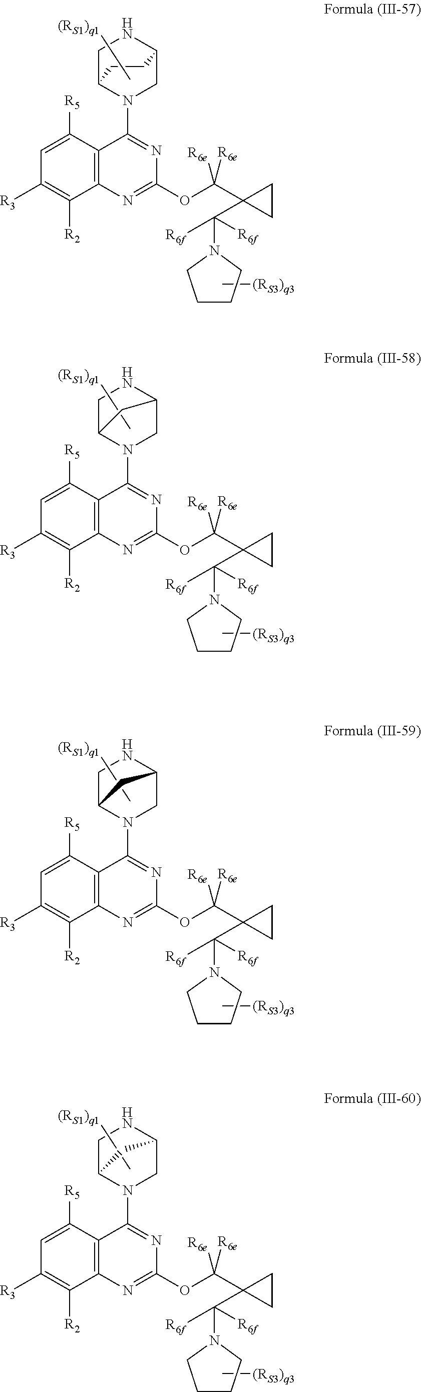

[8] The compound of any one of [1] to [7], wherein,
R₃ is selected from phenyl or naphthyl, wherein said Phenyl or naphthyl is optionally independently substituted with one or more R₃₁;
R₃₁ at each occurrence is independently selected from halogen, —C₁₋₆alkyl, haloC₁₋₆alkyl, haloC₁₋₆alkoxy, —C₂₋₆alkenyl, —C₂₋₆alkynyl, —CN, —N(R₇₅)₂, —OR₇₅, —SR₇₅, —S(═O)R₇₆, —S(═O)₂R₇₆, 3-6 membered cycloalkyl, or 3-6 membered heterocyclyl, wherein said —C₁₋₆alkyl, haloC₁₋₆alkyl, haloC₁₋₆alkoxy, —C₂₋₆alkenyl, —C₂₋₆alkynyl, 3-6 membered cycloalkyl, or 3-6 membered heterocyclyl is optionally independently substituted with 1, 2, or 3 substituents selected from halogen, —C₁₋₃alkyl, haloC₁₋₃alkyl, haloC₁₋₃alkoxy, —C₂₋₃alkenyl, —C₂₋₃alkynyl, —OR₇₇, —SR₇₇, —S(═O)R₇₈, —S(═O)₂R₇₈, 3-6 membered cycloalkyl, or 3-6 membered heterocyclyl;

Each of (R₇₅ and R₇₇) at each occurrence is independently selected from hydrogen, —C₁₋₆alkyl, —C₂₋₆alkenyl, or —C₂₋₆alkynyl;

Each of (R₇₆ and R₇₈) at each occurrence is independently selected from hydrogen, —C₁₋₃alkyl, —C₂₋₃alkenyl, or —C₂₋₃alkynyl;

Preferably, R₃₁ at each occurrence is independently selected from —OH, —C≡CH, —F, —Cl, —CH₃, —CH₂CH₃, —S—CH₃, —O—CH₃, —CF₃,

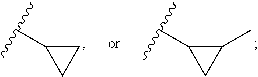

Preferably, R₃ is selected from

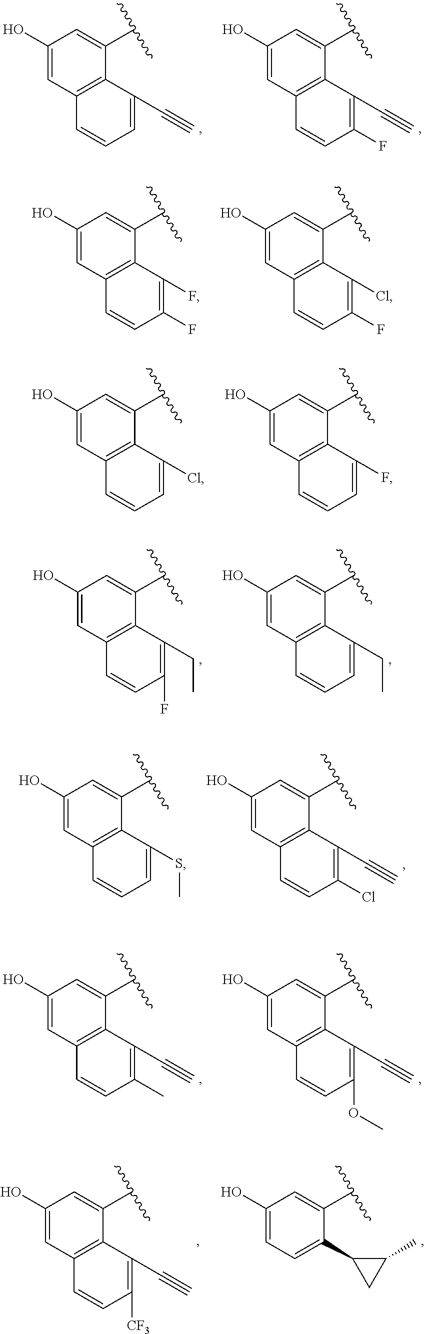

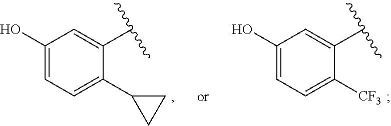 [SMILES: *c1cc(O)ccc1C1CC1 ; *c1cc(O)ccc1C(F)(F)F]

More preferably, R₃ is selected from

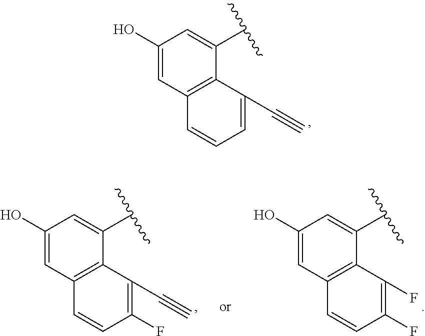 [SMILES: *c1cc(O)cc2cccc(C#C)c12 ; *c1cc(O)cc2ccc(F)c(C#C)c12 ; *c1cc(O)cc2ccc(F)c(F)c12]

[9] The compound of any one of [1] to [8], wherein,
the compound is selected from any one of formula (IV-1) to (IV-180):

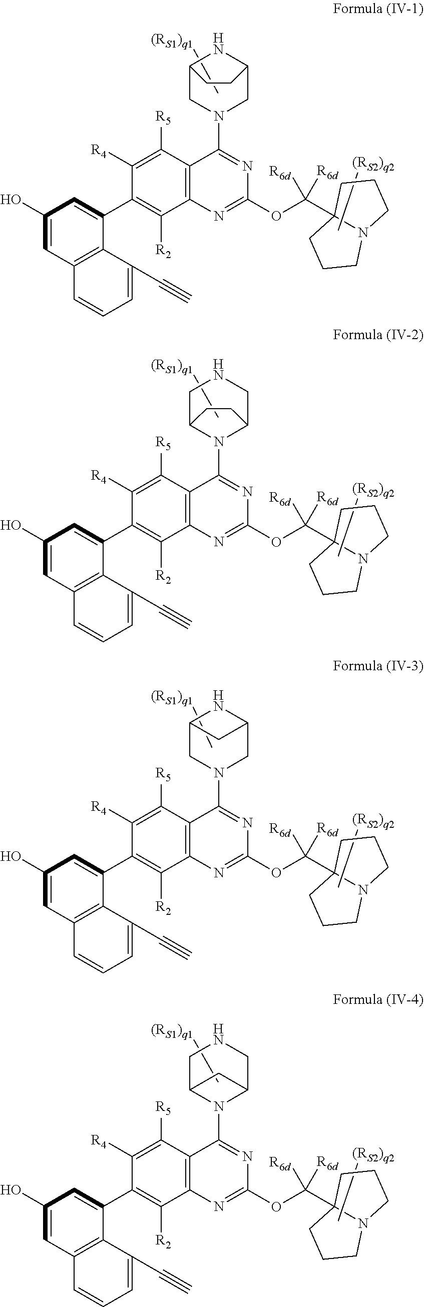

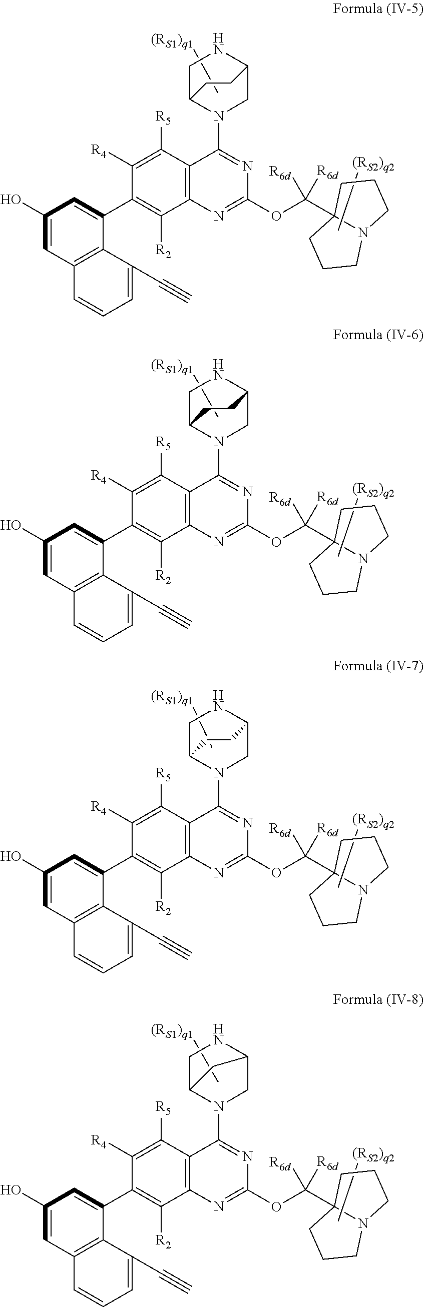

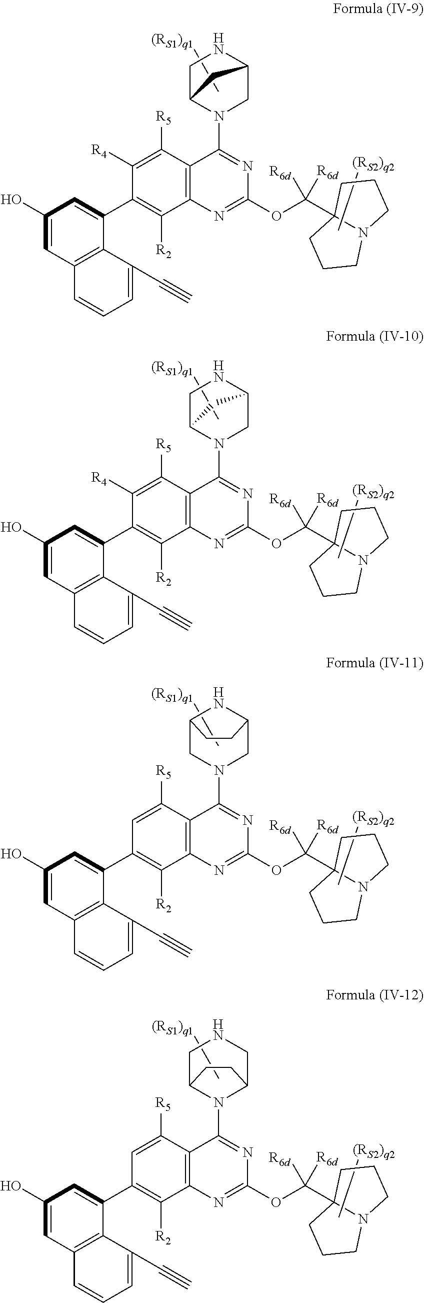

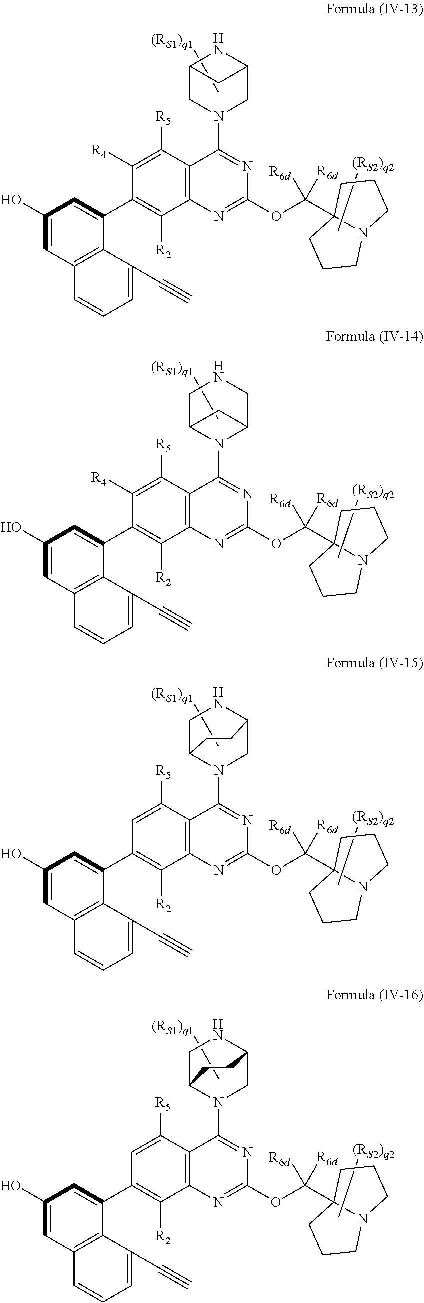

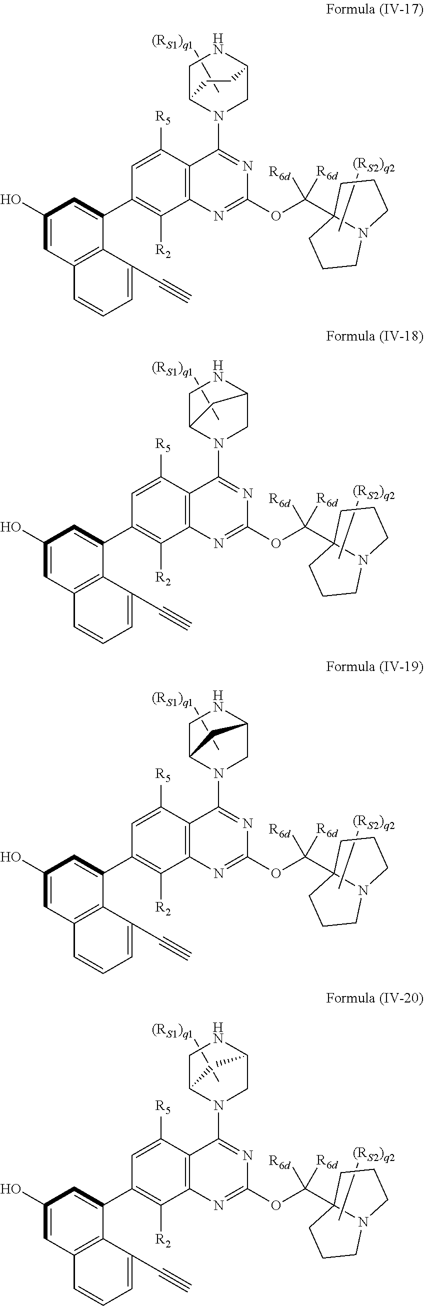

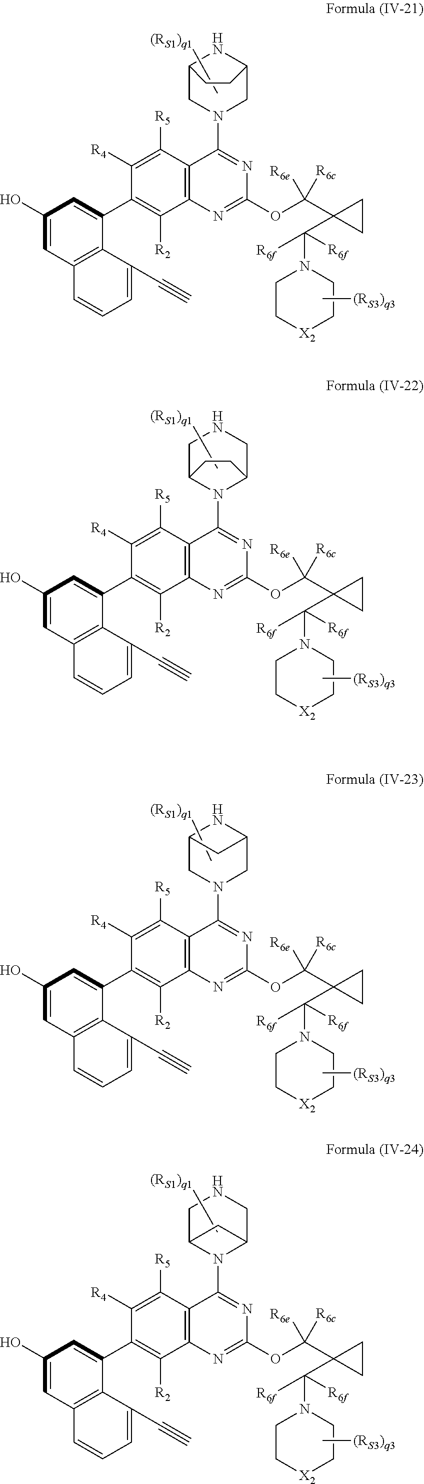

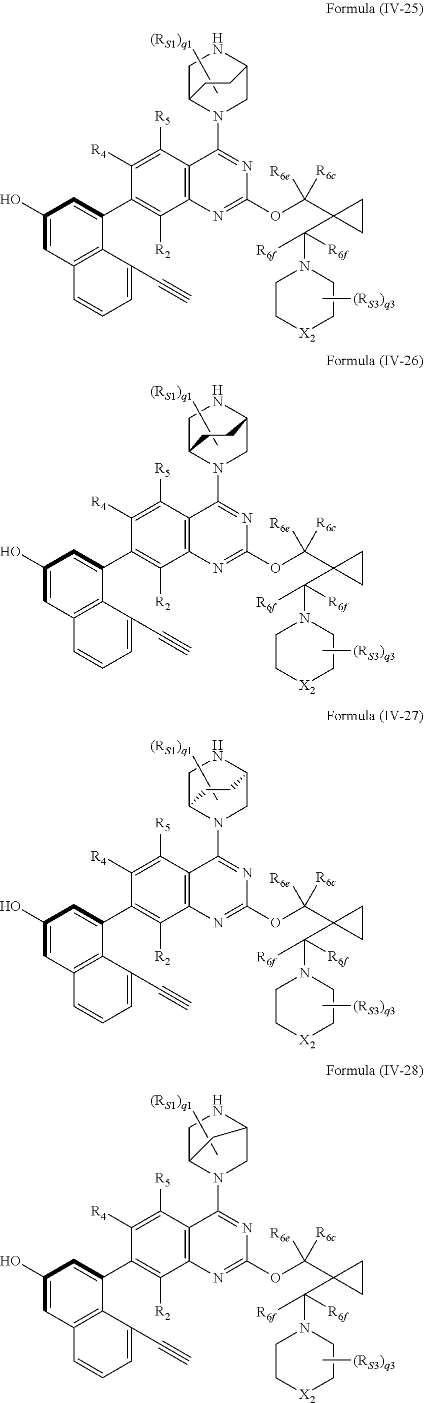

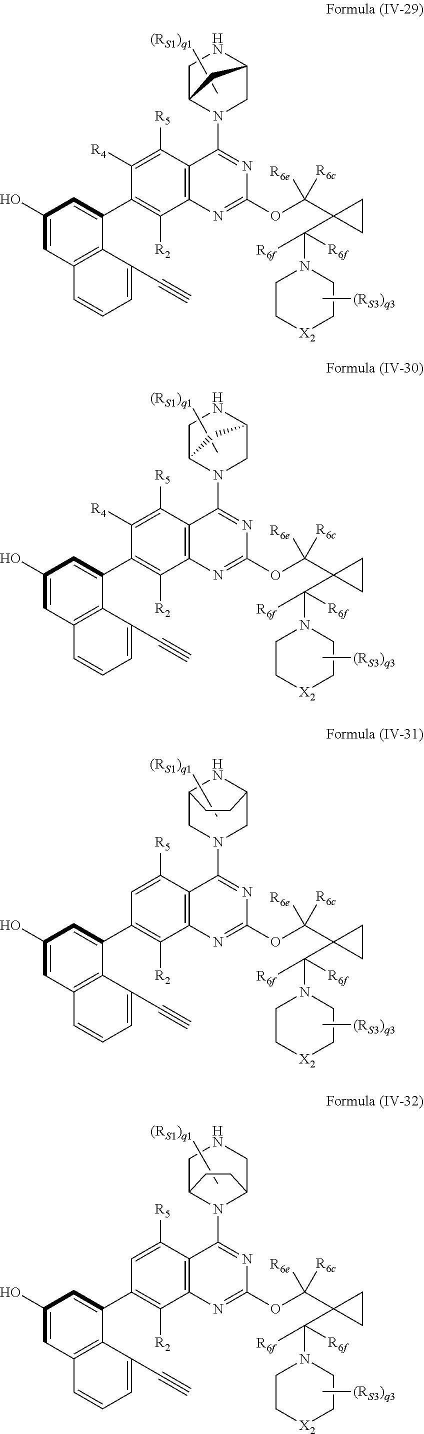

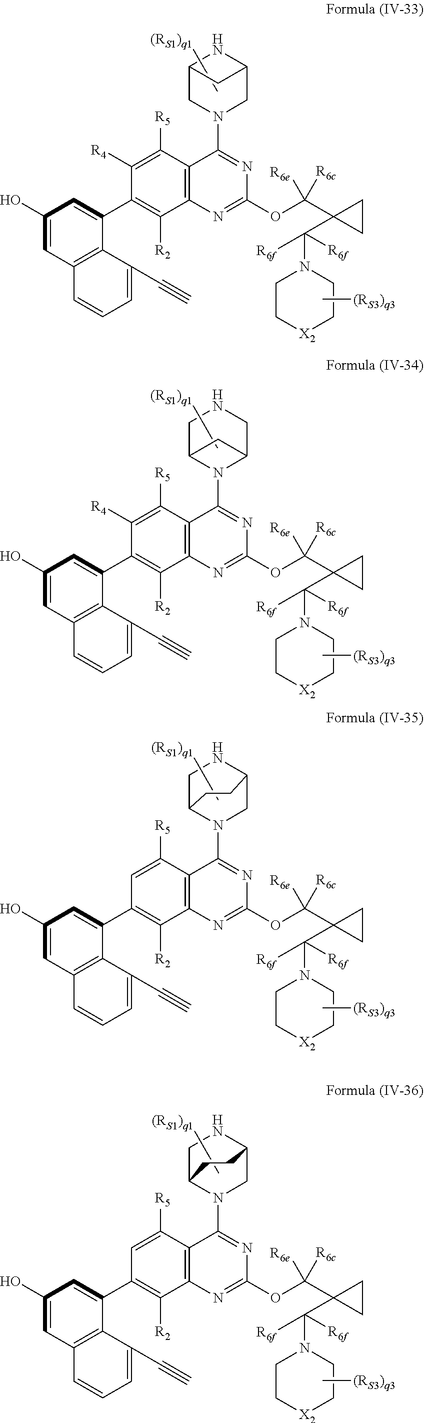

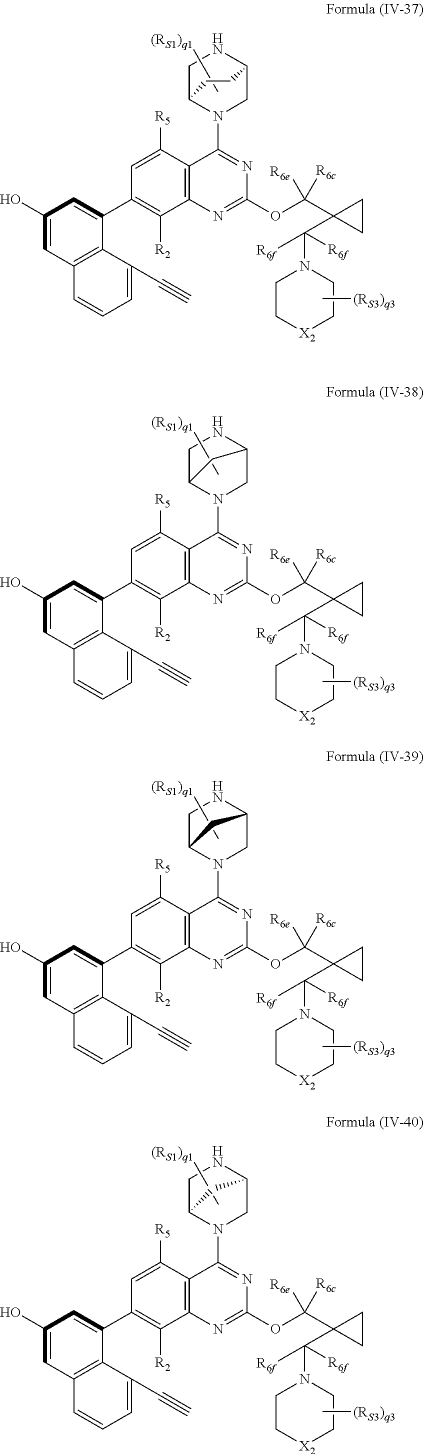

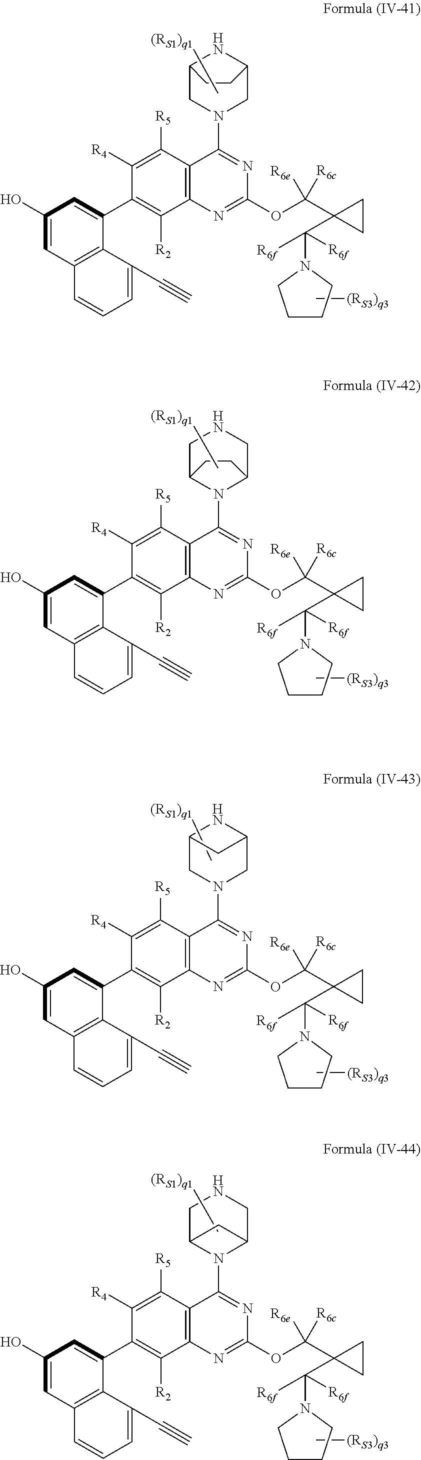

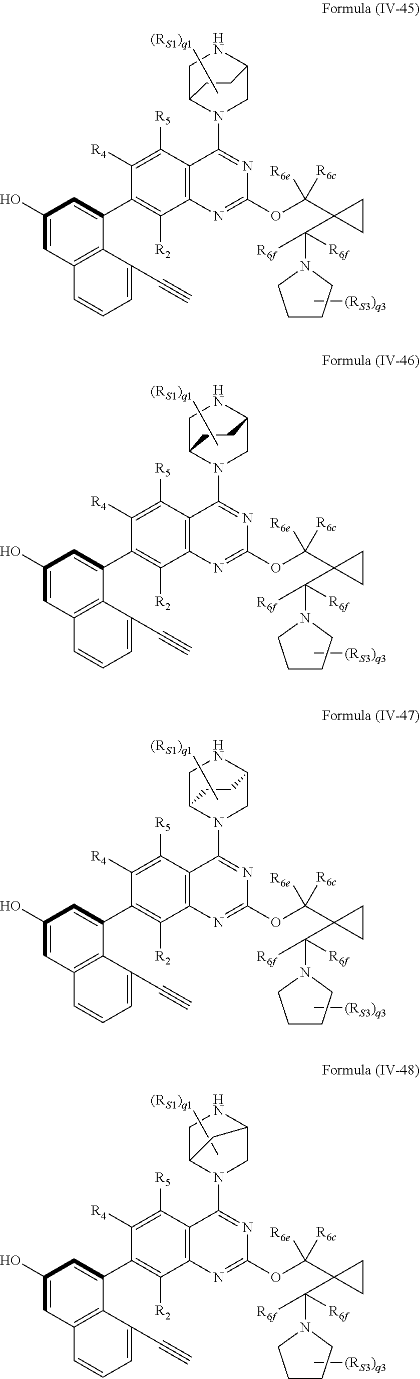

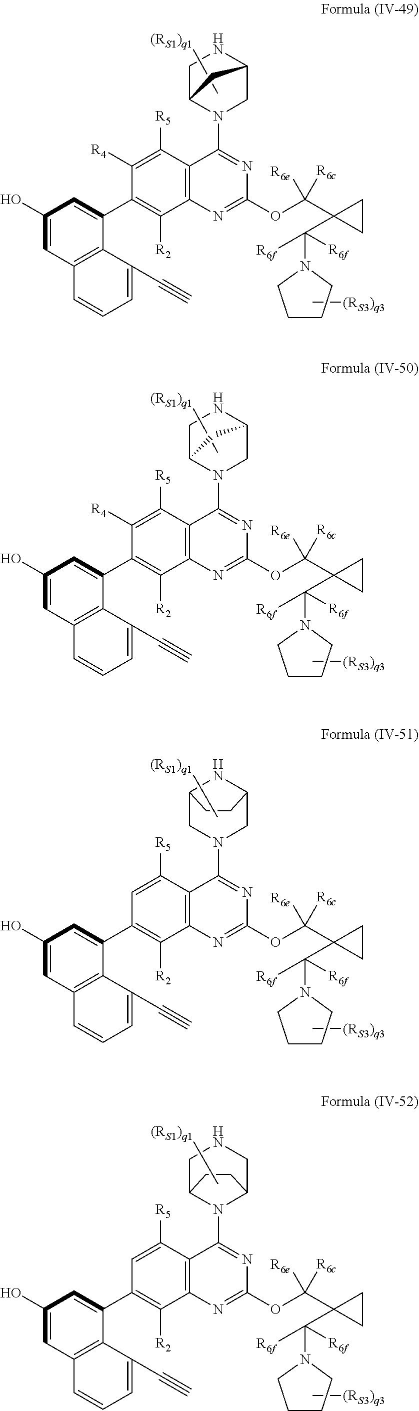

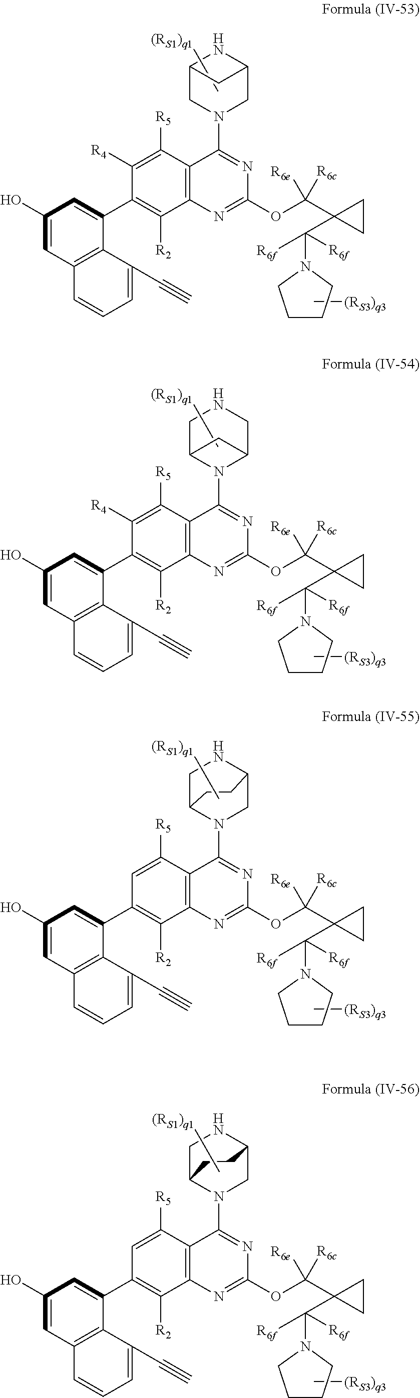

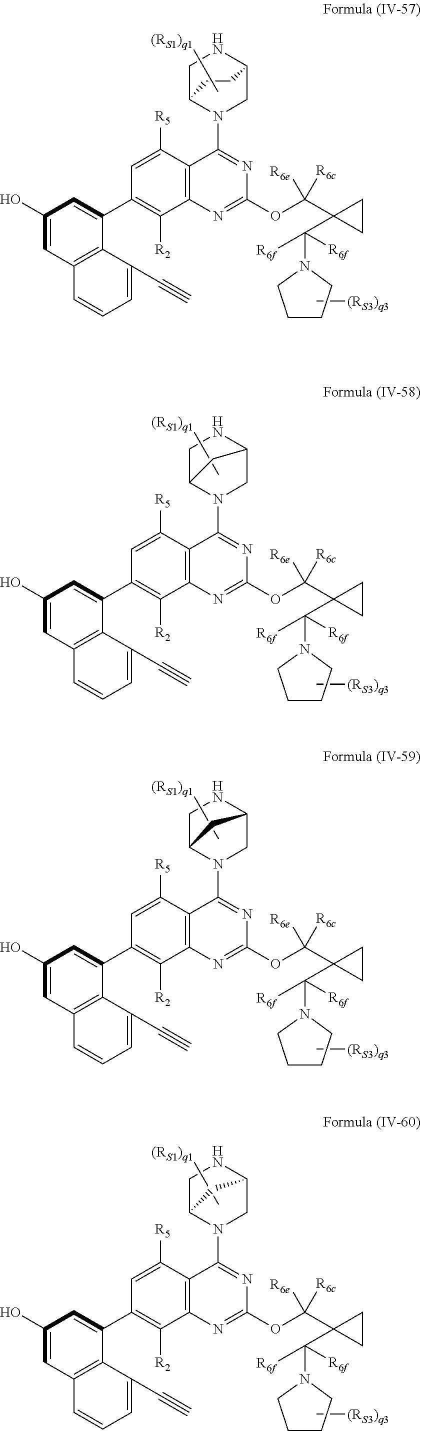

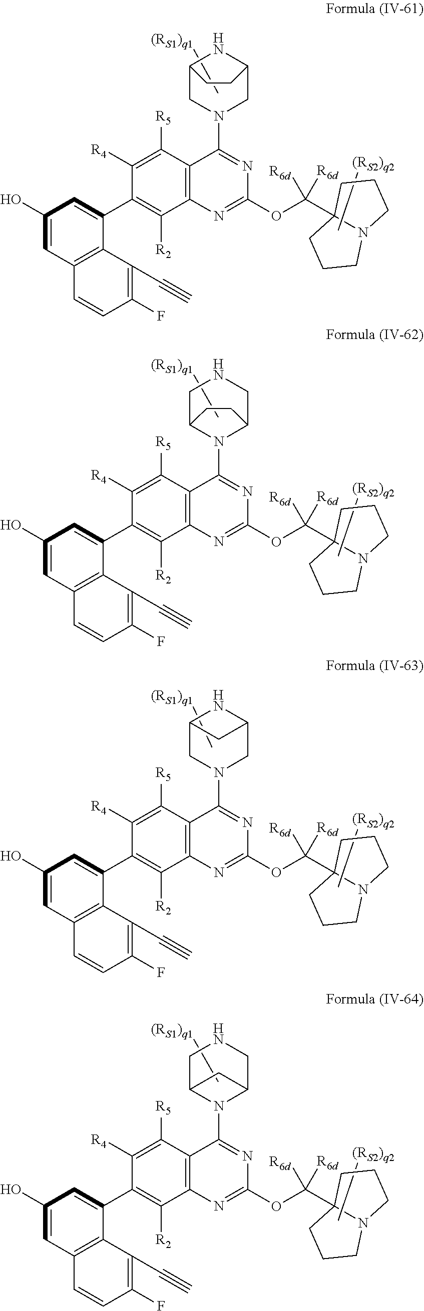

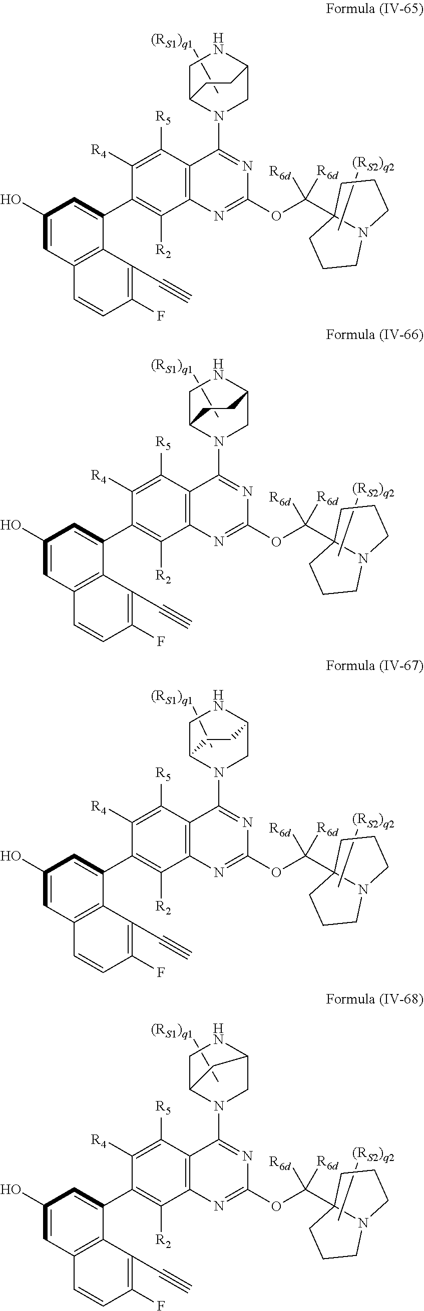

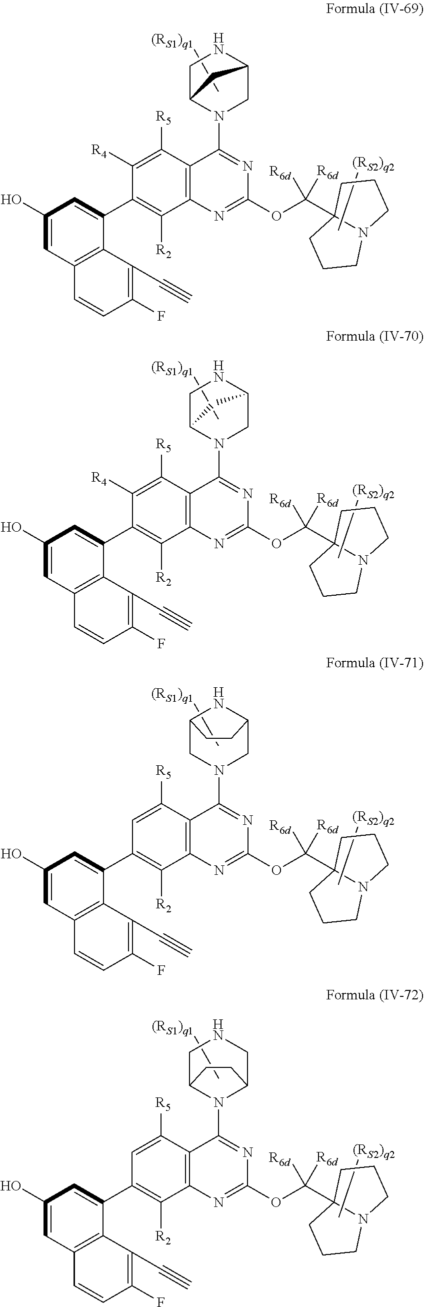

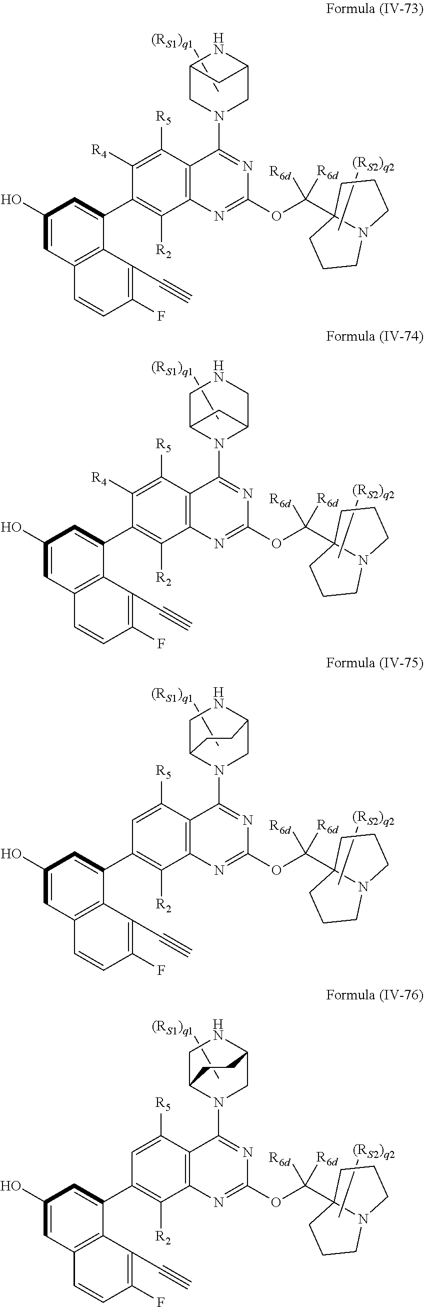

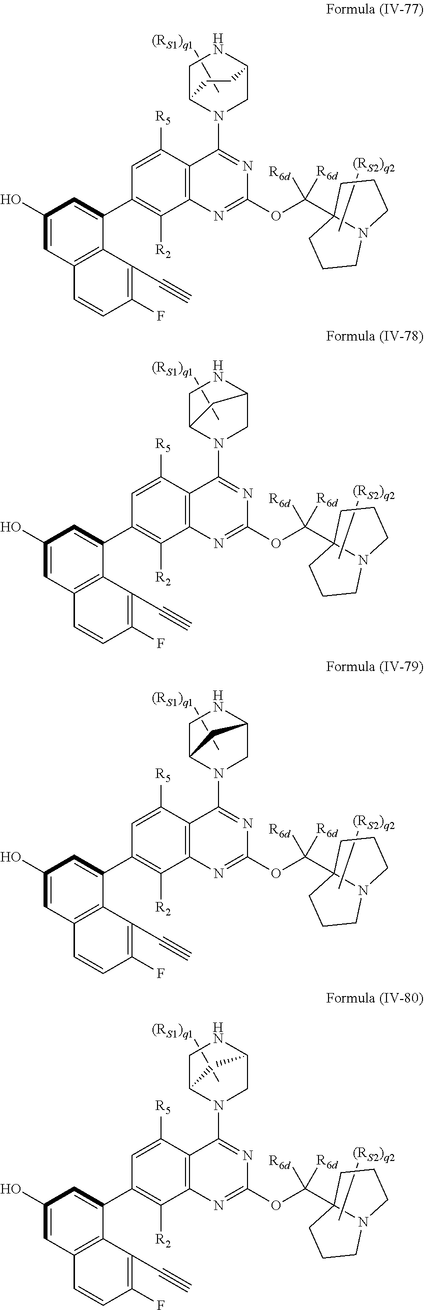

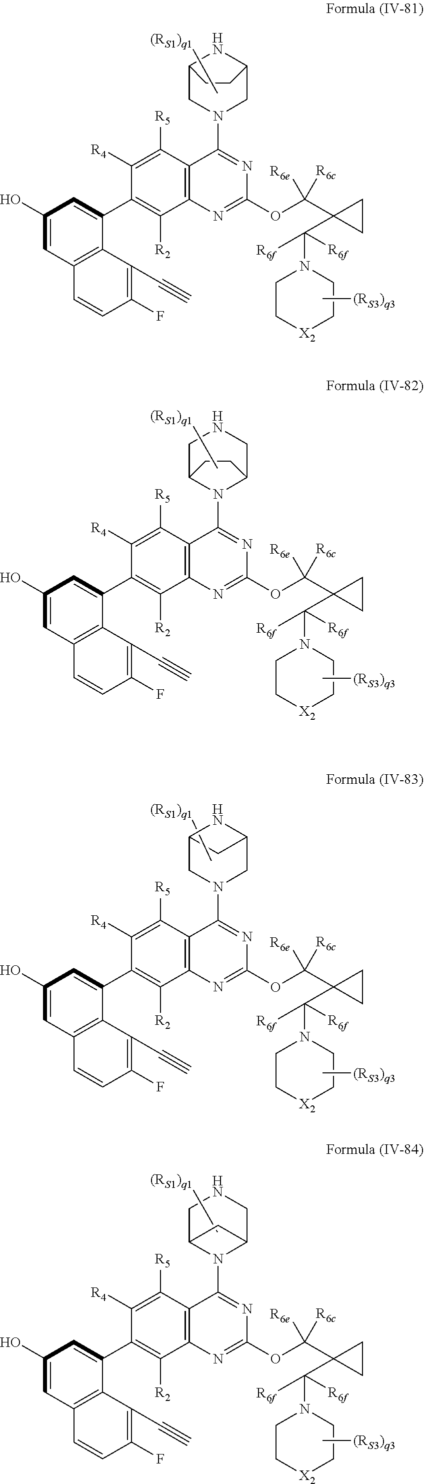

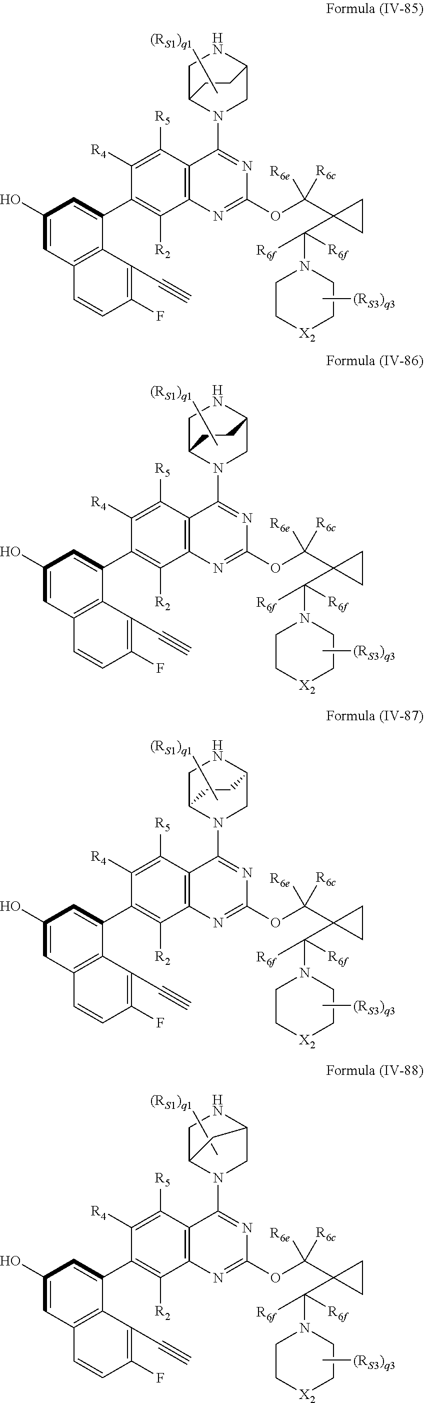

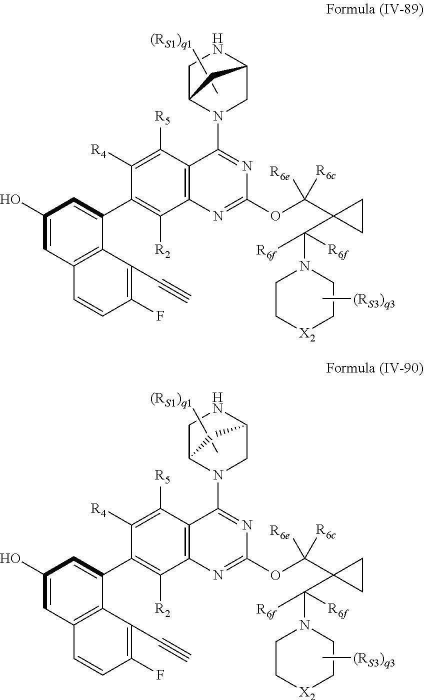

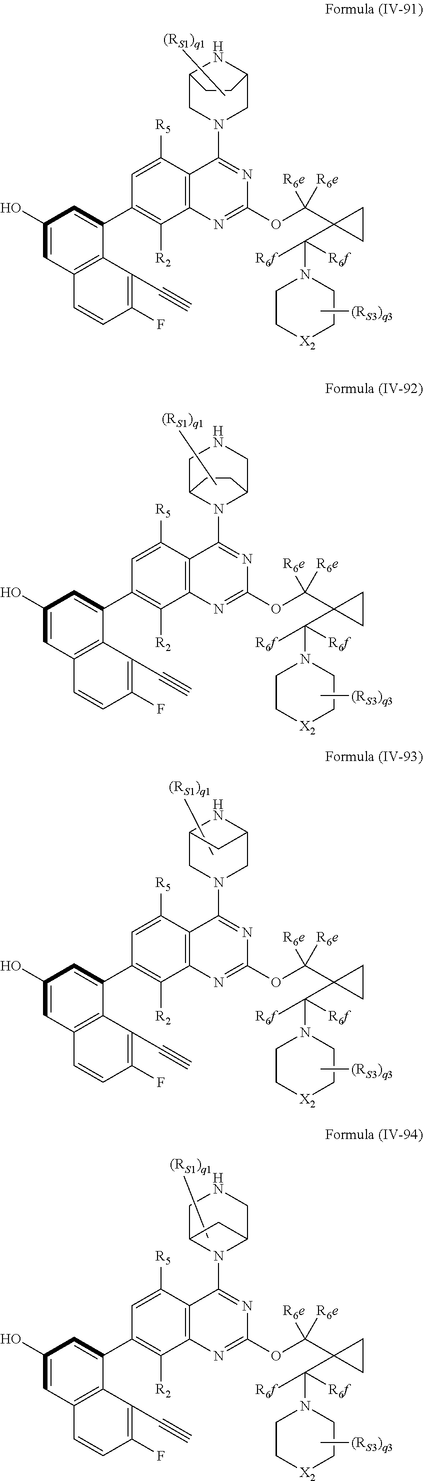

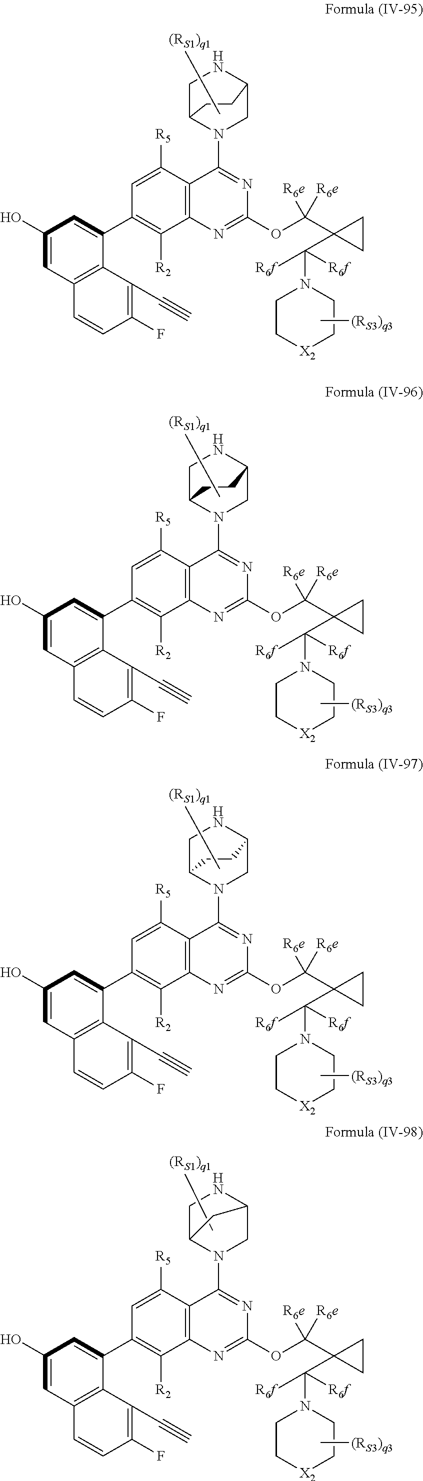

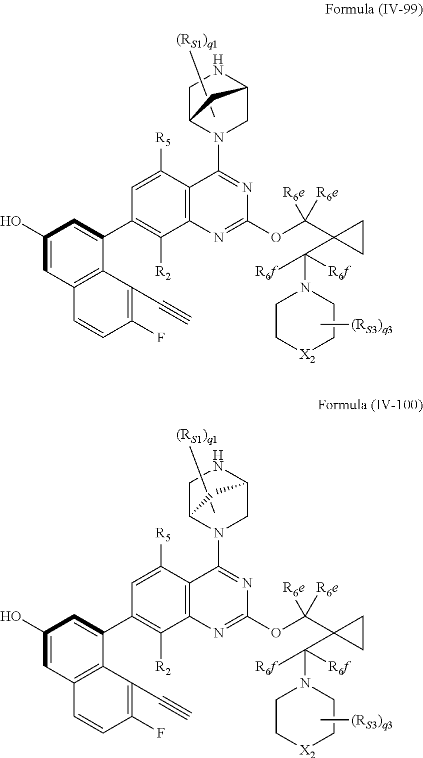

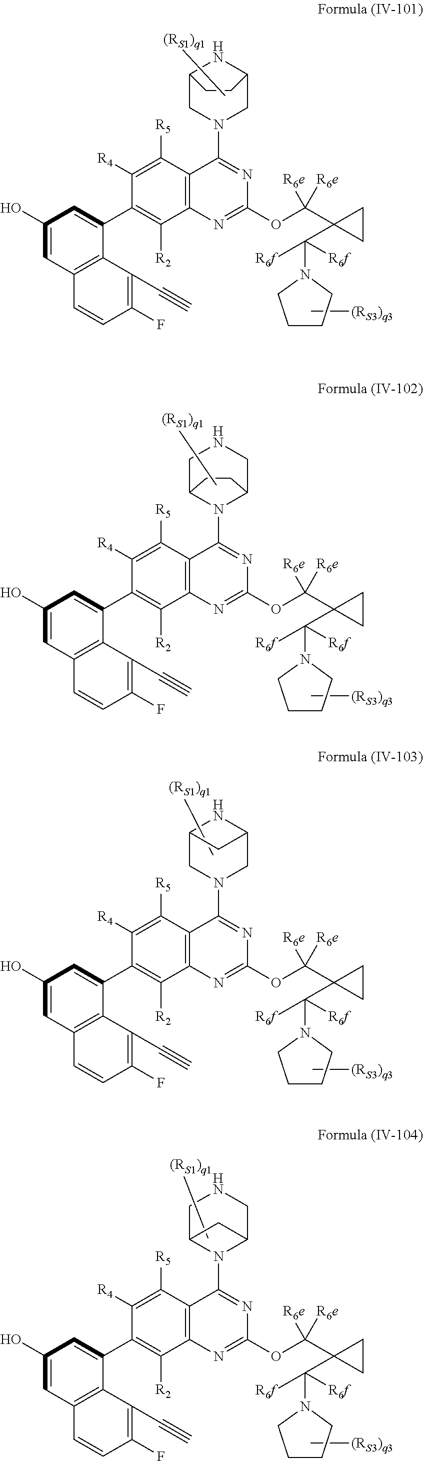

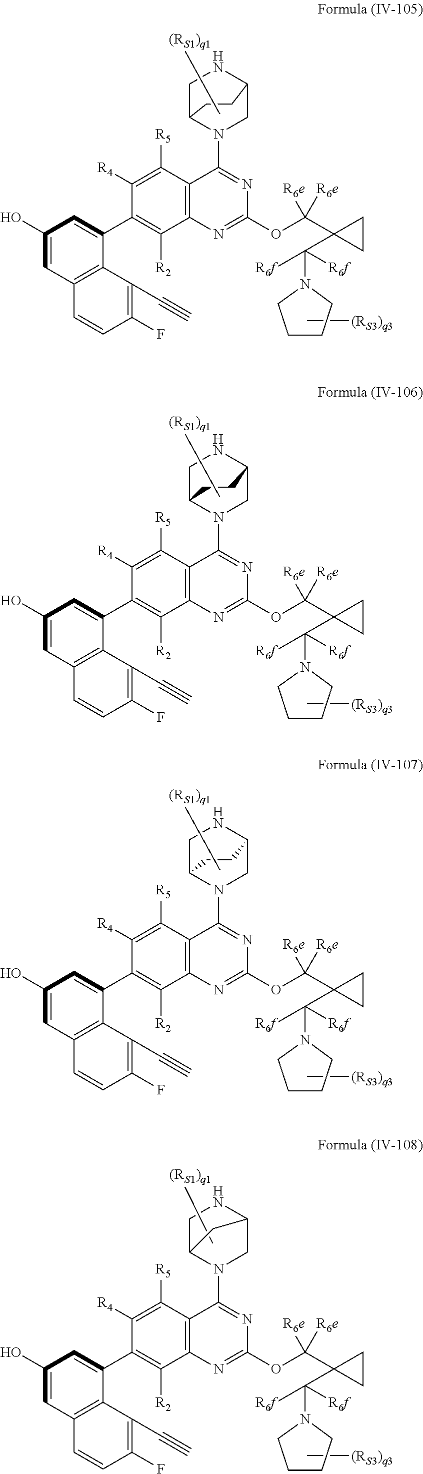

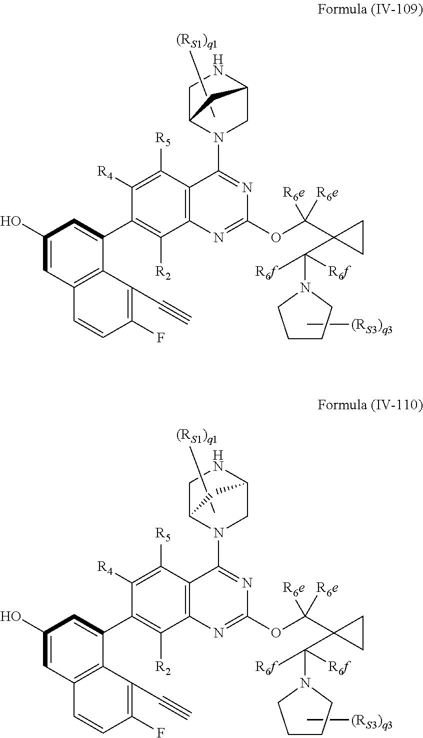

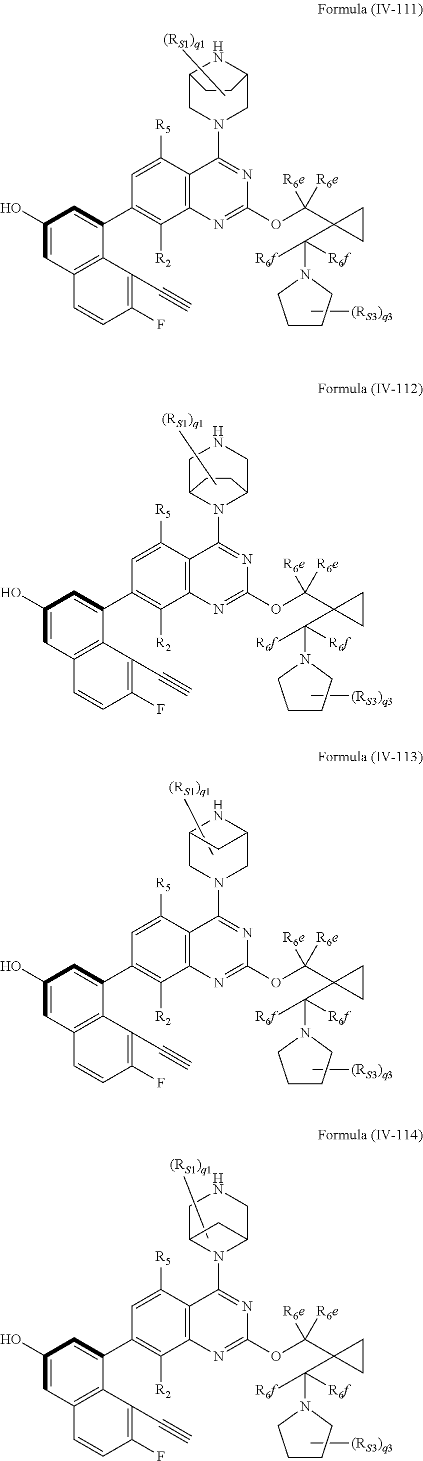

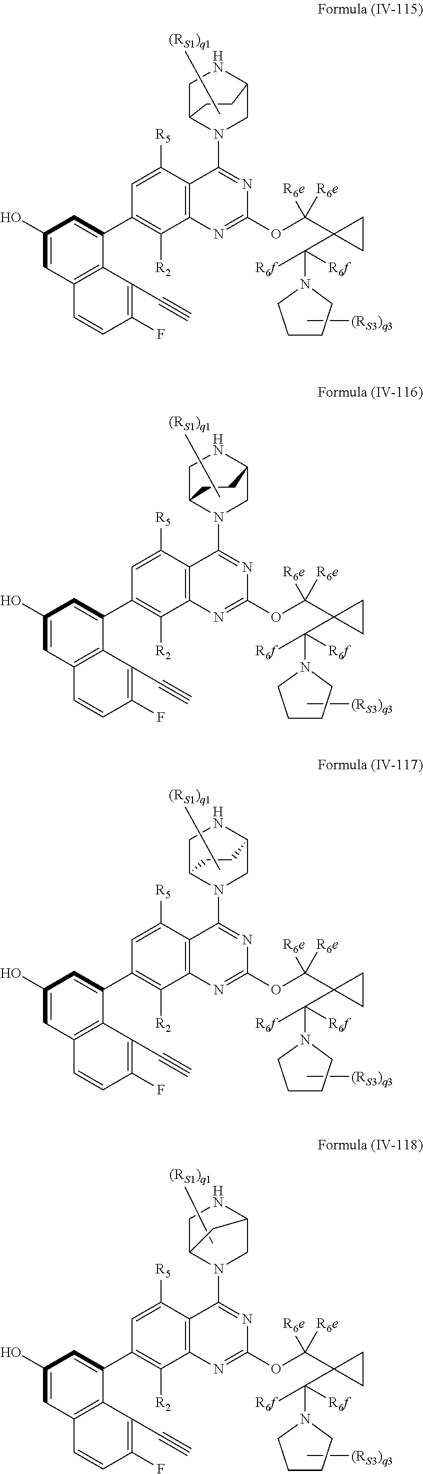

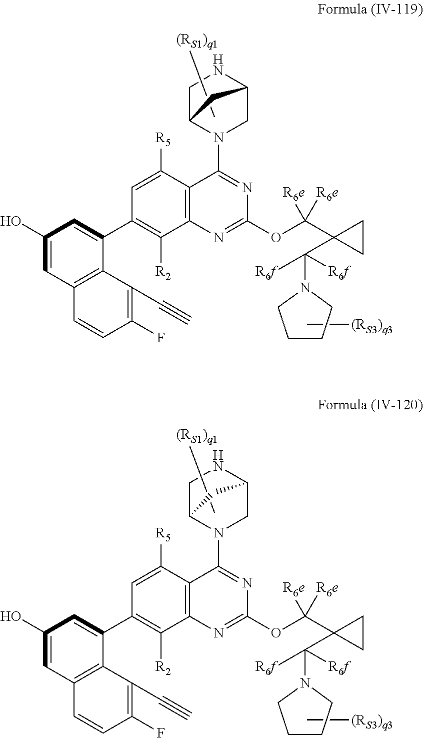

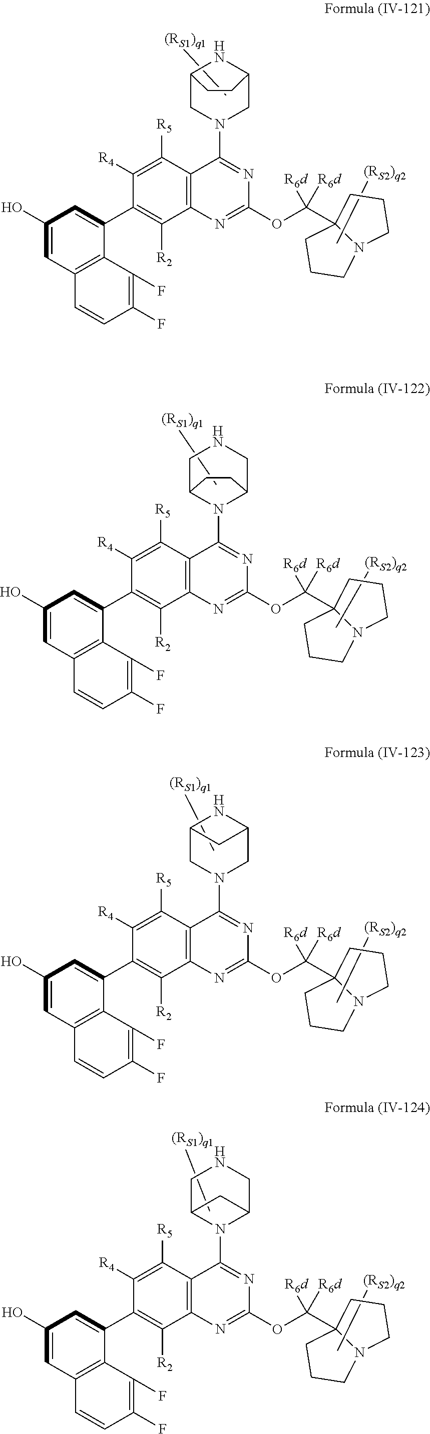

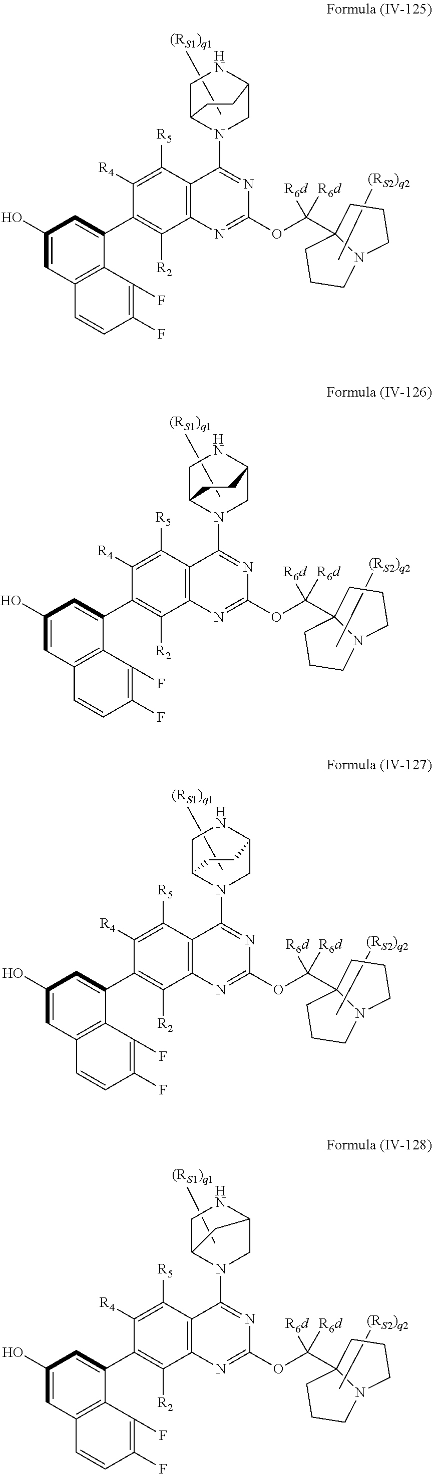

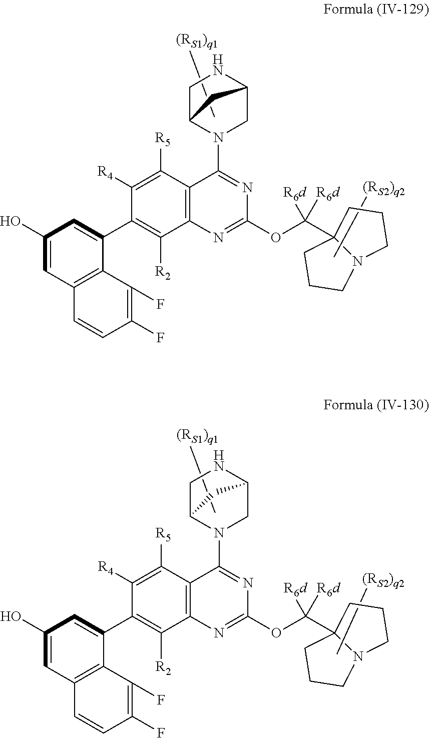

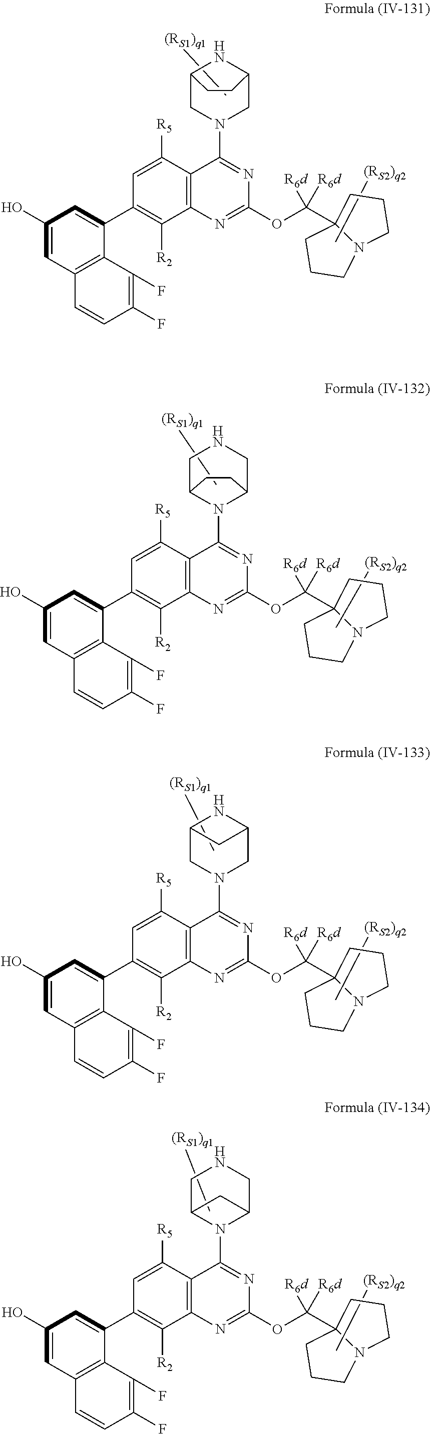

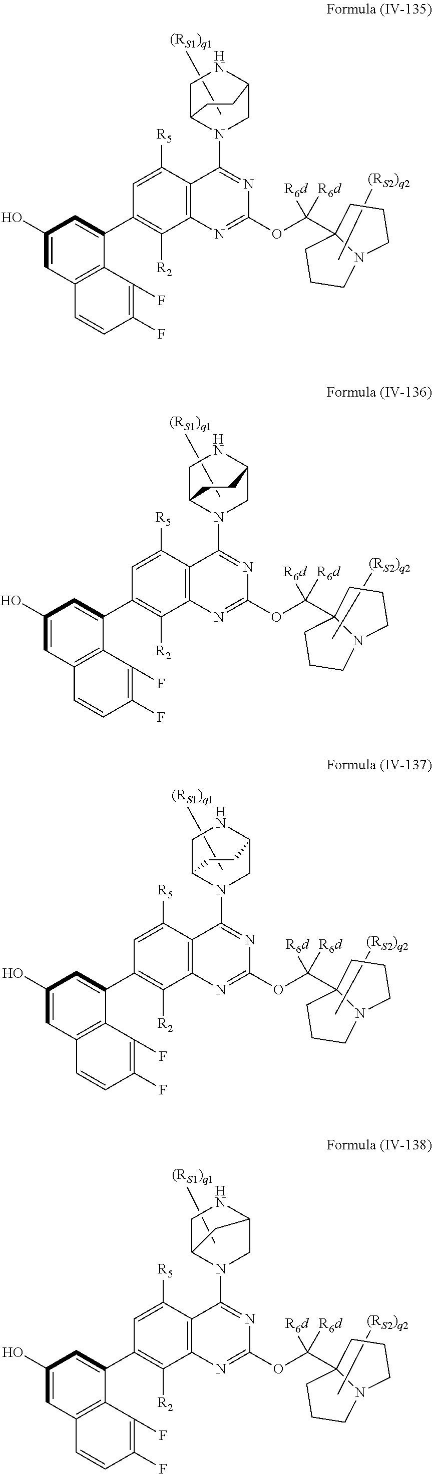

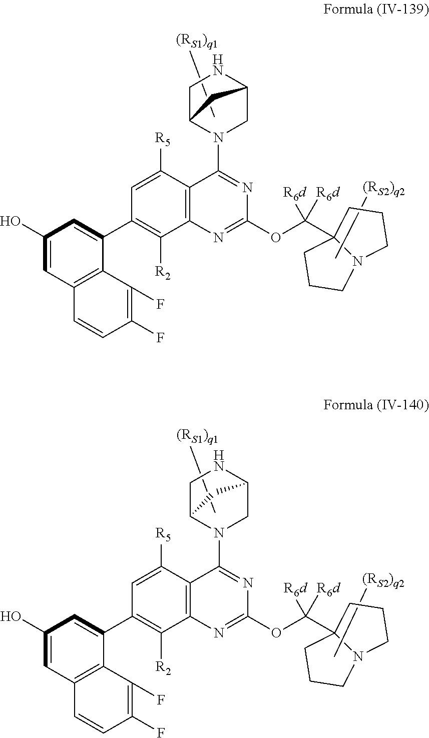

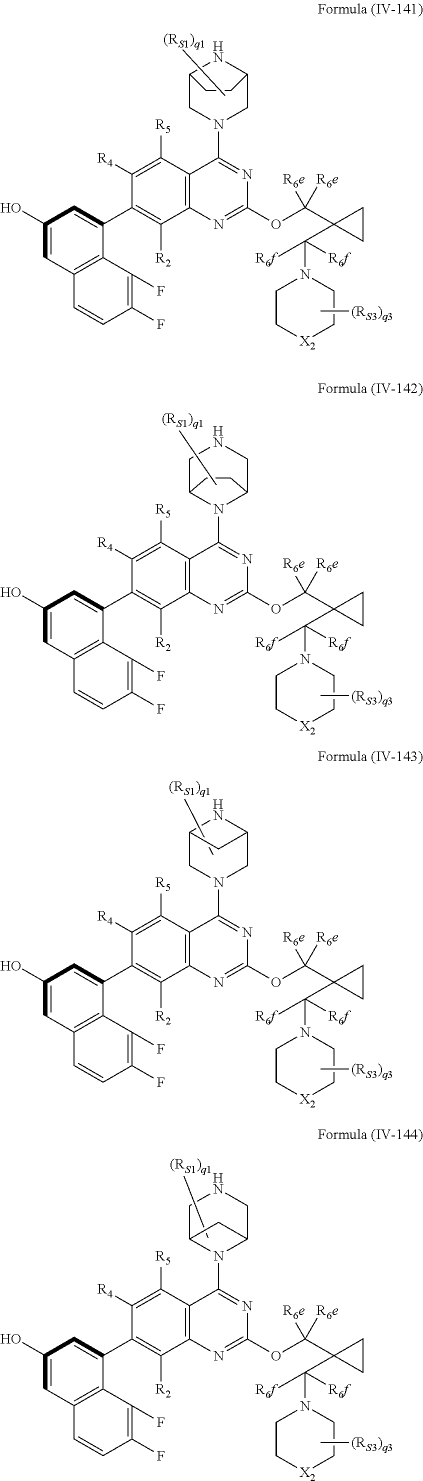

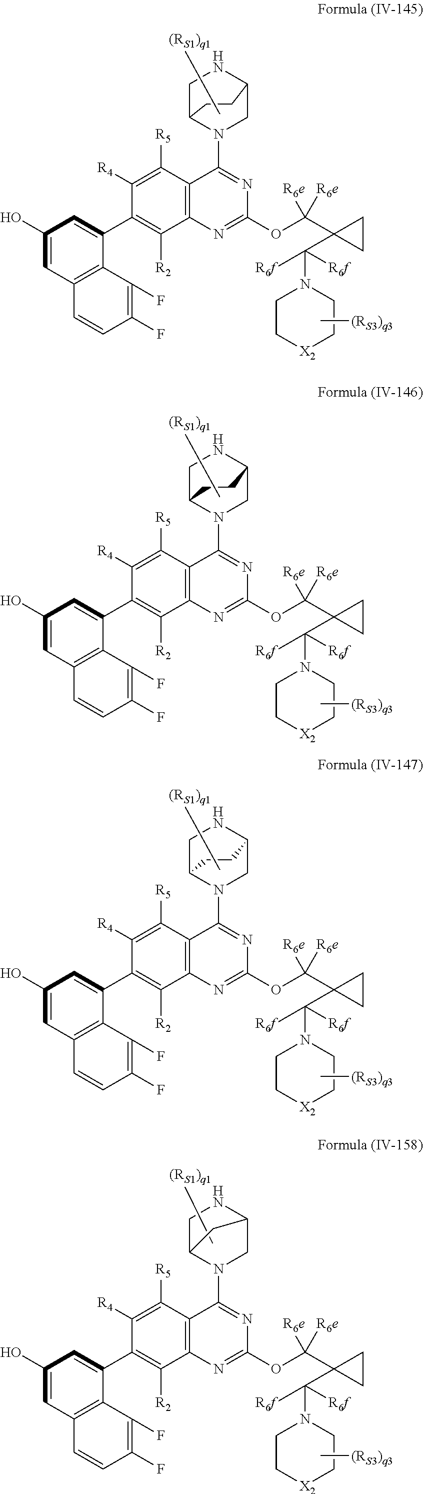

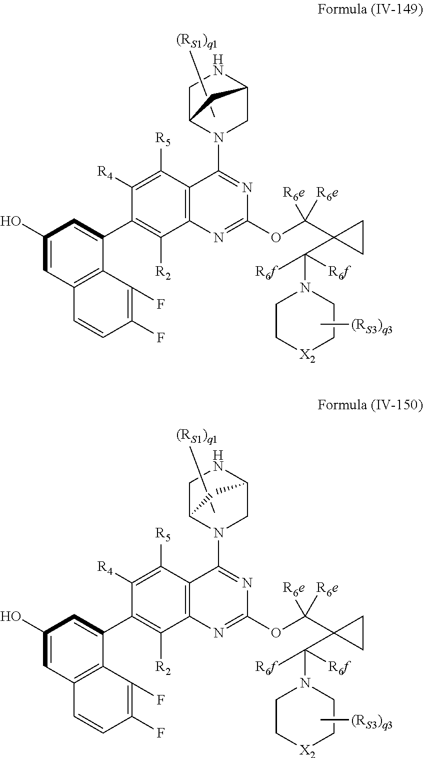

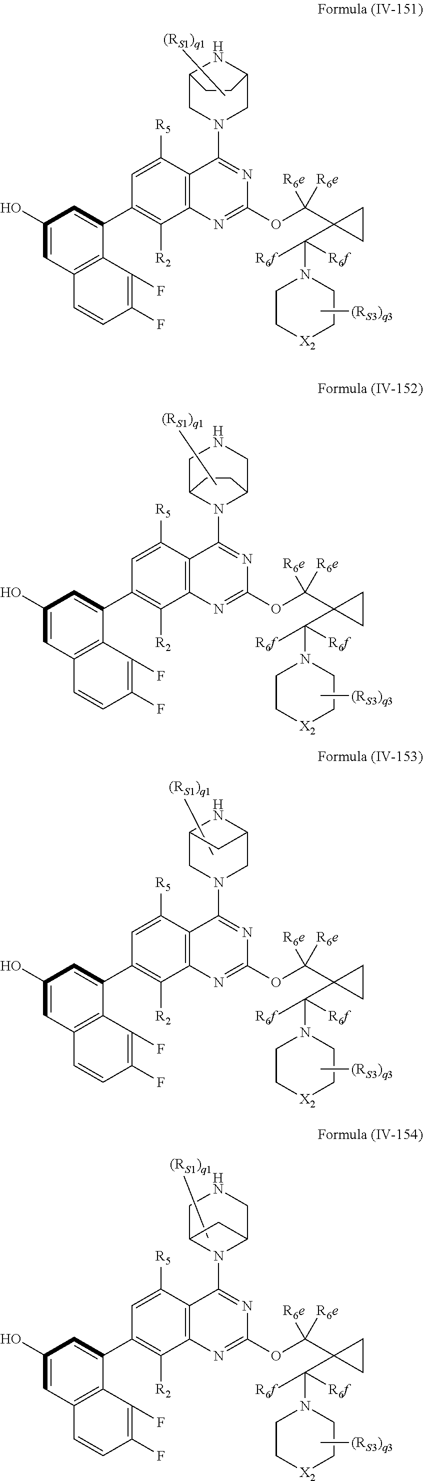

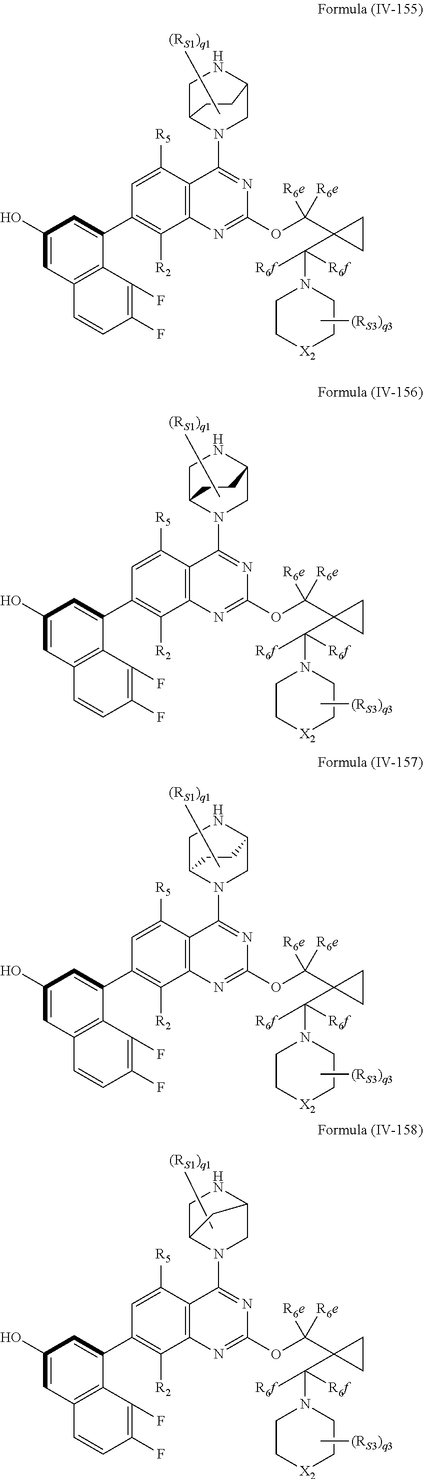

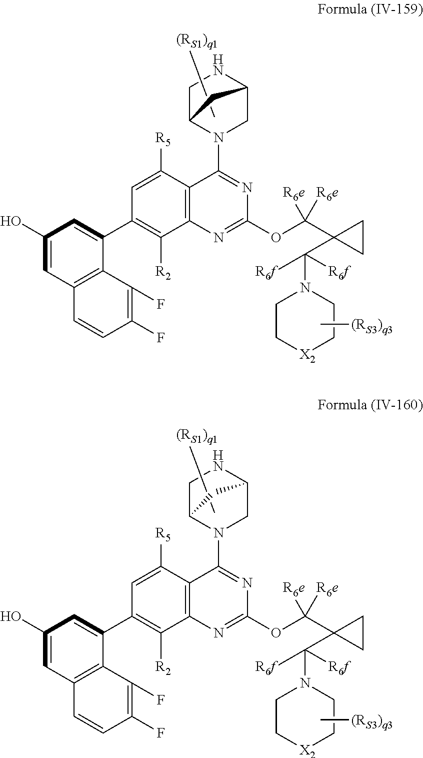

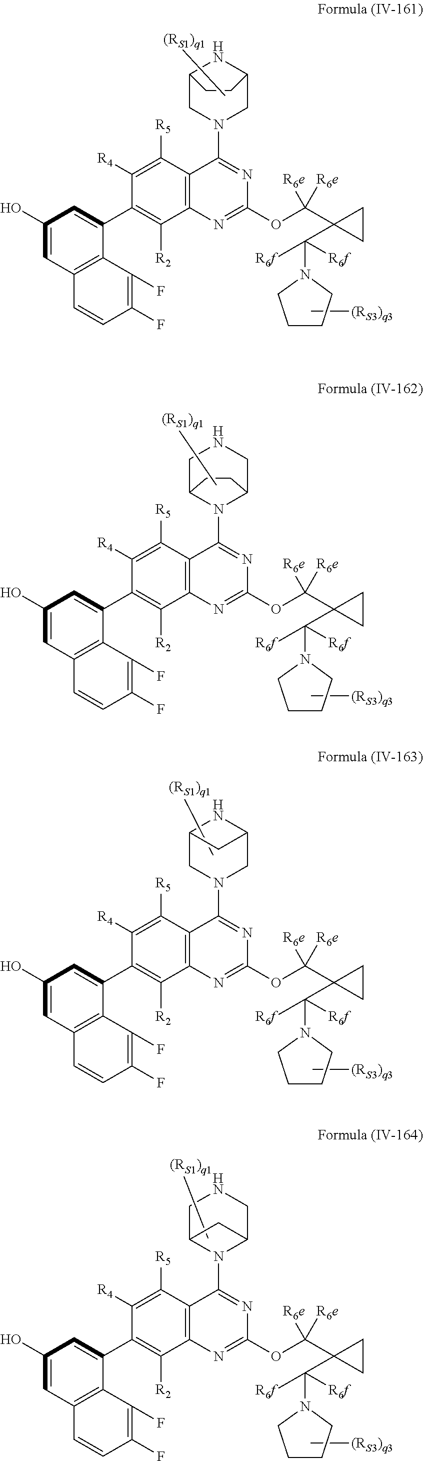

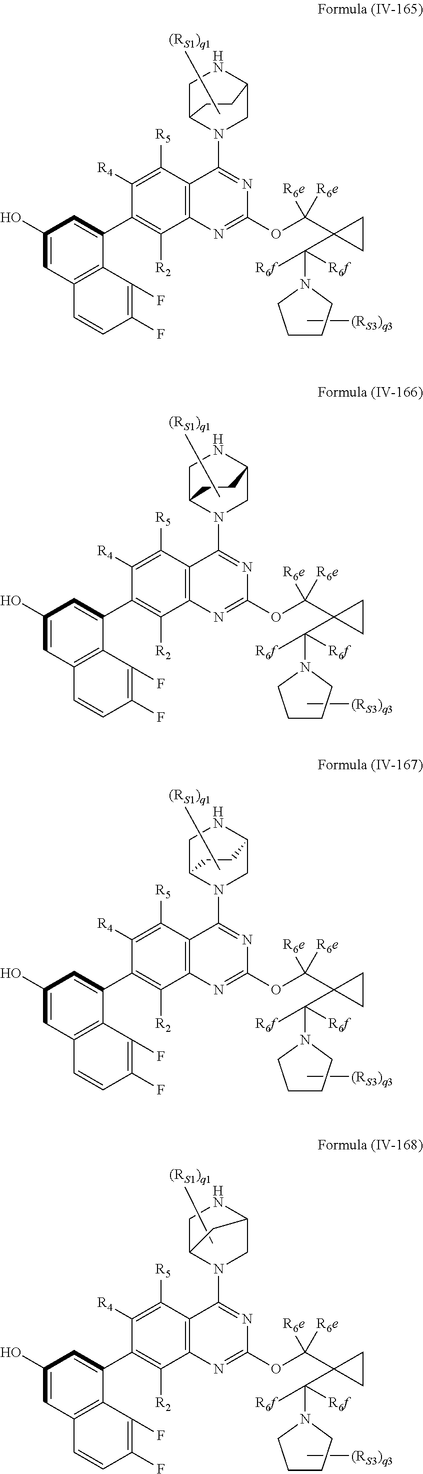

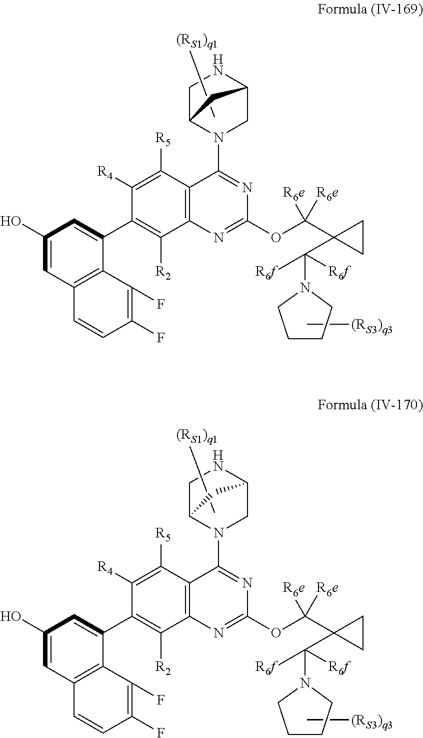

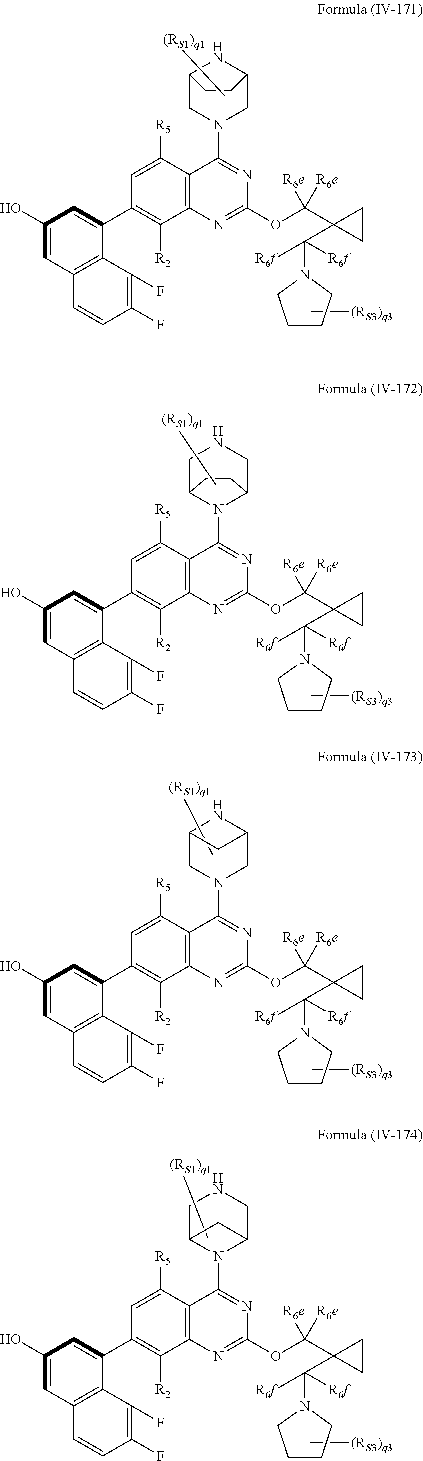

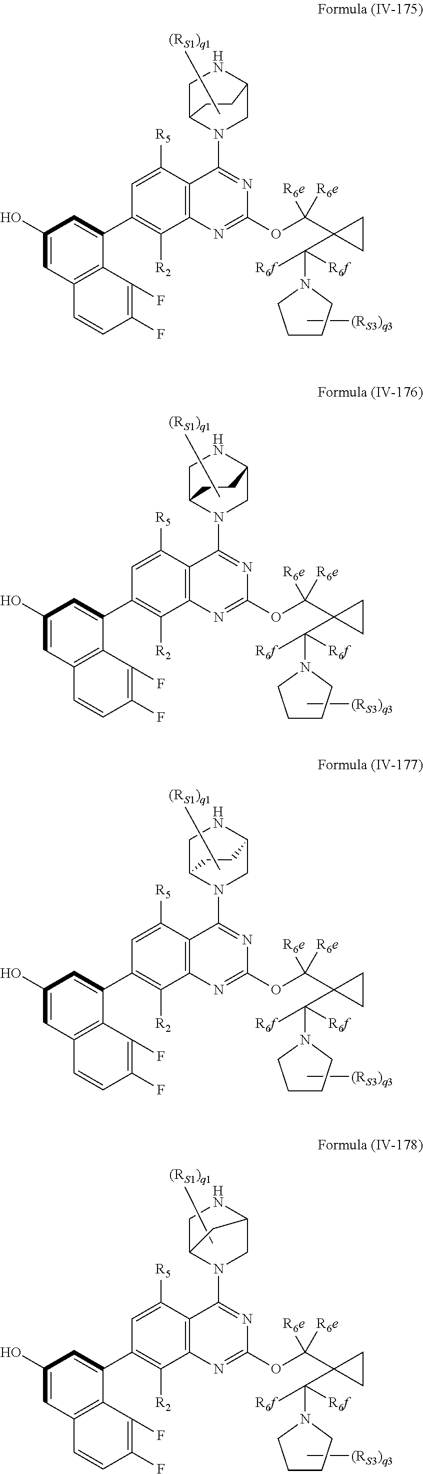

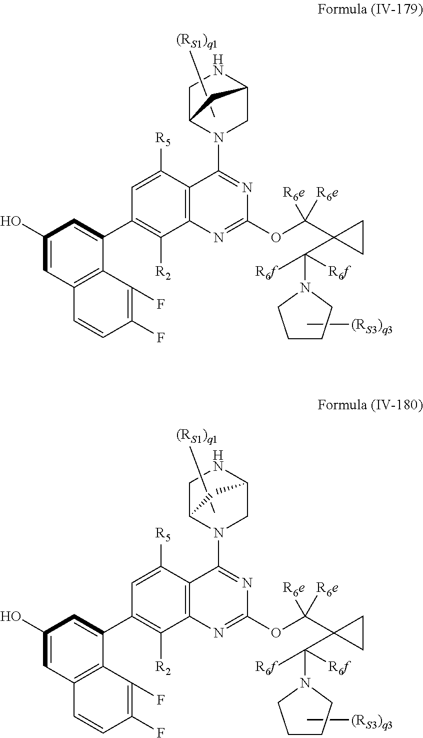

[10] The compound of any one of [1] to [9], wherein,

Each of R_{S2} at each occurrence is independently selected from halogen, —C₁₋₆alkyl, haloC₁₋₆alkyl, haloC₁₋₆alkoxy, —C₂₋₆alkenyl, —C₂₋₆alkynyl, —CN, —N(R₆₆)₂, —OR₆₆, —SR₆₆, —S(═O)R₆₇, —S(═O)₂R₆₇, —C(═O)R₆₇, —C(═O)OR₆₆, —OC(═O)R₆₇, —C(═O)N(R₆₆)₂, —NR₆₆C(═O)R₆₇, —OC(═O)OR₆₆, —NR₆₆C(═O)OR₆₆, —OC(═O)N(R₆₆)₂, —NR₆₆C(═O)N(R₆₆)₂, 3-6 membered cycloalkyl, or 4-8 membered heterocyclyl, wherein said —C₁₋₆alkyl, haloC₁₋₆alkyl, haloC₁₋₆alkoxy, —C₂₋₆alkenyl, —C₂₋₆alkynyl, 3-8 membered cycloalkyl, or 4-8 membered heterocyclyl is optionally independently substituted with 1, 2, or 3 substituents selected from halogen, —C₁₋₆alkyl, haloC₁₋₆alkyl, haloC₁₋₆alkoxy, —C₂₋₆alkenyl, —C₂₋₆alkynyl, haloC₂₋₆alkynyl, —CN, —N(R₆₈)₂, —OR₆₈, —SR₆₈, —S(═O)R₆₉, —S(═O)₂R₆₉, —C(═O)R₆₉, —C(═O)OR₆₈, —OC(═O)R₆₉, —C(═O)N(R₆₈)₂, —NR₆₈C(═O)R₆₉, —OC(═O)OR₆₈, —NR₆₈C(═O)OR₆₈, —OC(═O)N(R₆₈)₂, —NR₆₈C(═O)N(R₆₈)₂, 3-6 membered cycloalkyl, or 3-6 membered heterocyclyl;

Each of (R₆₇ and R₆₉) at each occurrence is independently selected from hydrogen, —C₁₋₆alkyl, —C₂₋₆alkenyl, or —C₂₋₆alkynyl, wherein said —C₁₋₆alkyl, —C₂₋₆alkenyl, or —C₂₋₆alkynyl is optionally independently substituted with 1, 2, or 3 substituents selected from halogen;

Each of (R₆₆ and R₆₈) at each occurrence is independently selected from hydrogen, halogen, —C₁₋₆alkyl, haloC₁₋₆alkyl, —C₂₋₆alkenyl, —C₂₋₆alkynyl, —C(═O)Rₐ, —C(═O)ORₐ, —C(═O)N(Rₐ)₂, 3-6 membered cycloalkyl, or 3-6 membered heterocyclyl, wherein said —C₁₋₆alkyl, haloC₁₋₆alkyl, —C₂₋₆alkenyl, —C₂₋₆alkynyl, 3-6 membered cycloalkyl, or 3-6 membered heterocyclyl is optionally independently substituted with one or more substituents selected from halogen, —C₁₋₃alkyl, haloC₁₋₃alkyl, haloC₁₋₃alkoxy, —C₂₋₃alkenyl, —C₂₋₃alkynyl, —CN, 3-6 membered cycloalkyl, or 3-6 membered heterocyclyl;

Optionally, each of (two R₆₆ and two R₆₈) independently together with the nitrogen atom to which they are both attached forms a 3-8 membered heterocyclic ring or a 5-10 membered heteroaryl ring, wherein, said 3-8 membered heterocyclic ring or 5-10 membered heteroaryl ring is optionally independently substituted with one or more R₆ₖ;

Each of R₆ₖ at each occurrence is independently selected from halogen, —C₁₋₃alkyl, haloC₁₋₃alkyl, haloC₁₋₃alkoxy, —C₂₋₃alkenyl, —C₂₋₃alkynyl, —CN, —NH₂, —NH(C₁₋₃alkyl), —N(C₁₋₃alkyl)₂, —OH, —O(C₁₋₃alkyl), —SH, —S(C₁₋₃alkyl), —S(═O)(C₁₋₃alkyl), —S(═O)₂(C₁₋₃alkyl), 3-6 membered cycloalkyl, or 3-6 membered heterocyclyl;

Preferably, each of R_{S2} at each occurrence is independently selected from

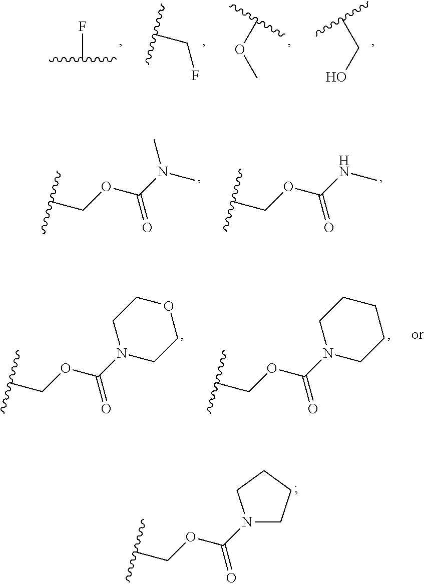

More preferably, each of R_{S2} at each occurrence is independently selected from H NNJ

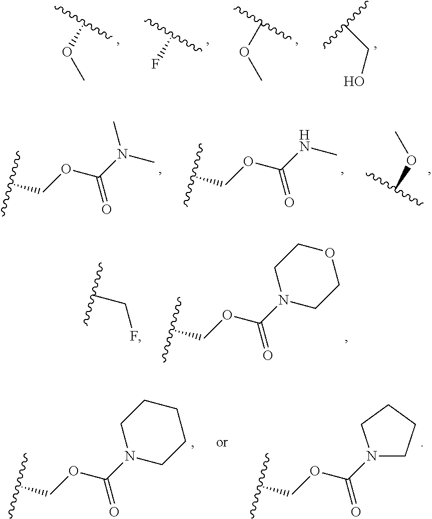

[11] The compound of any one of [1] to [10], wherein,

Wherein the moiety of

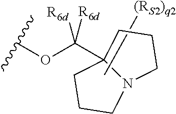

is selected from

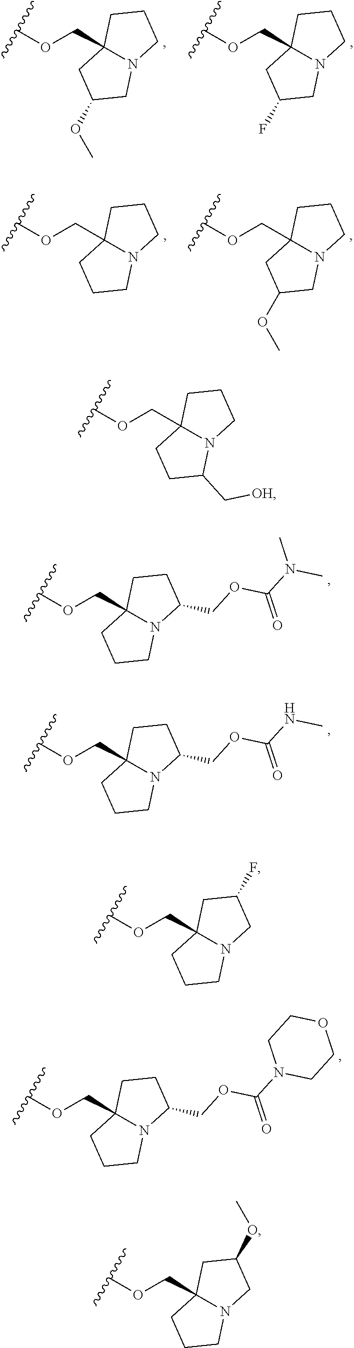

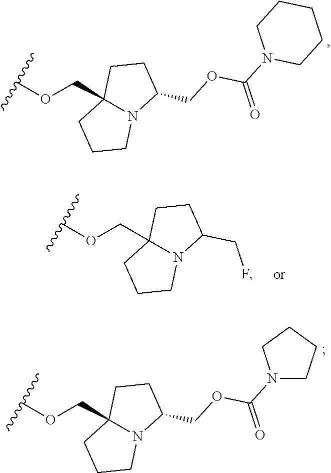 [SMILES: *OC[C@]12CCCN1[C@@H](COC(=O)N1CCCCC1)CC2 ; *OCC12CCCN1C(CF)CC2 ; *OC[C@]12CCCN1[C@@H](COC(=O)N1CCCC1)CC2]

Preferably, the moiety of

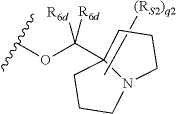

is selected from

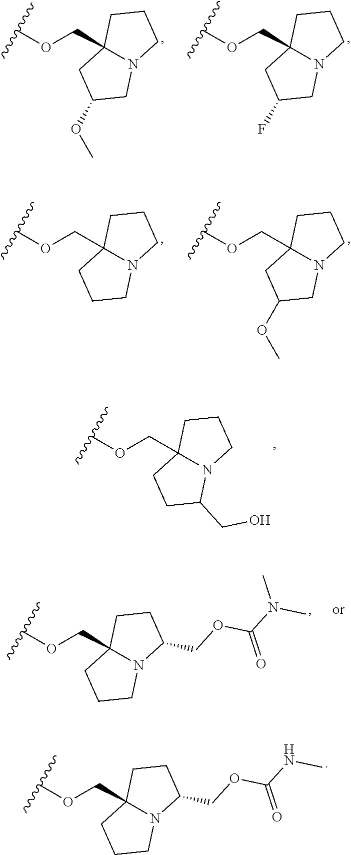

[12] The compound ofany one of[1] to [11], wherein,

Each of R_{S3} at each occurrence is independently selected from halogen, —C₁₋₆alkyl, haloC₁₋₆alkyl, haloC₁₋₆alkoxy, —C₂₋₆alkenyl, —C₂₋₆alkynyl, —CN, —N(R₆₆)₂, —OR₆₆, —SR₆₆, —S(═O)R₆₇, —S(═O)₂R₆₇, —C(═O)R₆₇, —C(═O)OR₆₆, —OC(═O)R₆₇, —C(═O)N(R₆₆)₂, —NR₆₆C(═O)R₆₇, —OC(═O)OR₆₆, —NR₆₆C(═O)OR₆₆, —OC(═O)N(R₆₆)₂, —NR₆₆C(═O)N(R₆₆)₂, 3-6 membered cycloalkyl, or 4-8 membered heterocyclyl, wherein said —C₁₋₆alkyl, haloC₁₋₆alkyl, haloC₁₋₆alkoxy, —C₂₋₆alkenyl, —C₂₋₆alkynyl, 3-8 membered cycloalkyl, or 4-8 membered heterocyclyl is optionally independently substituted with 1, 2, or 3 substituents selected from halogen, —C₁₋₆alkyl, haloC₁₋₆alkyl, haloC₁₋₆alkoxy, —C₂₋₆alkenyl, —C₂₋₆alkynyl, haloC₂₋₆alkynyl, —CN, —N(R₆₈)₂, —OR₆₈, —SR₆₈, —S(═O)R₆₉, —S(═O)₂R₆₉, —C(═O)R₆₉, —C(═O)OR₆₈, —OC(═O)R₆₉, —C(═O)N(R₆₈)₂, —NR₆₈C(═O)R₆₉, —OC(═O)OR₆₈, —NR₆₈C(═O)OR₆₈, —OC(═O)N(R₆₈)₂, —NR₆₈C(═O)N(R₆₈)₂, 3-6 membered cycloalkyl, or 3-6 membered heterocyclyl;

Each of (R₆₇ and R₆₉) at each occurrence is independently selected from hydrogen, —C₁₋₆alkyl, —C₂₋₆alkenyl, or —C₂₋₆alkynyl, wherein said —C₁₋₆alkyl, —C₂₋₆alkenyl, or —C₂₋₆alkynyl is optionally independently substituted with 1, 2, or 3 substituents selected from halogen;

Each of (R₆₆ and R₆₈) at each occurrence is independently selected from hydrogen, halogen, —C₁₋₆alkyl, haloC₁₋₆alkyl, —C₂₋₆alkenyl, —C₂₋₆alkynyl, —S(═O)Rₐ, —S(═O)₂Rₐ, —C(═O)Rₐ, —C(═O)ORₐ, —C(═O)N(Rₐ)₂, 3-6 membered cycloalkyl, or 3-6 membered heterocyclyl; wherein said —C₁₋₆alkyl, haloC₁₋₆alkyl, —C₂₋₆alkenyl, —C₂₋₆alkynyl, 3-6 membered cycloalkyl, or 3-6 membered heterocyclyl is optionally independently substituted with one or more substituents selected from halogen, —C₁₋₃alkyl, haloC₁₋₃alkyl, haloC₁₋₃alkoxy, —C₂₋₃alkenyl, —C₂₋₃alkynyl, —CN, —N(R_{c})₂, —OR_{c}, —SR_{c}, —S(═O)R_{d}, —S(═O)₂R_{d}, —C(═O)R_{d}, —C(═O)OR_{c}, —OC(═O)R_{d}, —C(═O)N(R_{c})₂, —NR_{c}C(═O)R_{d}, 3-6 membered cycloalkyl, or 3-6 membered heterocyclyl;

Each of (Rₐ, R_{c} and R_{d}) at each occurrence is independently selected from hydrogen, —C₁₋₃alkyl, haloC₁₋₃alkyl, haloC₁₋₃alkoxy, —C₂₋₃alkenyl, —C₂₋₃alkynyl, 3-6 membered cycloalkyl, or 3-6 membered heterocyclyl;

Preferably, each of R_{S3} at each occurrence is independently selected from

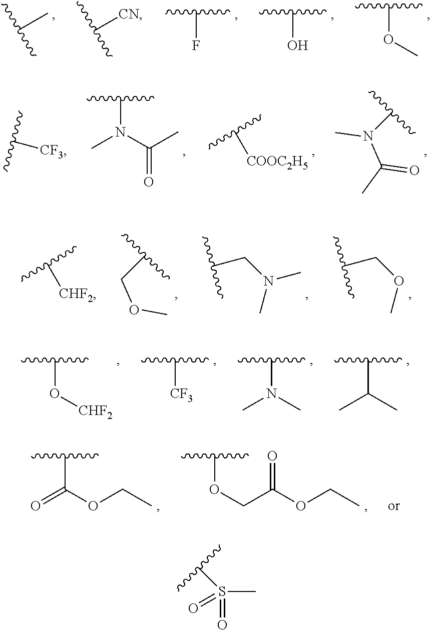

More preferably, each of R_{S3} at each occurrence is independently selected from

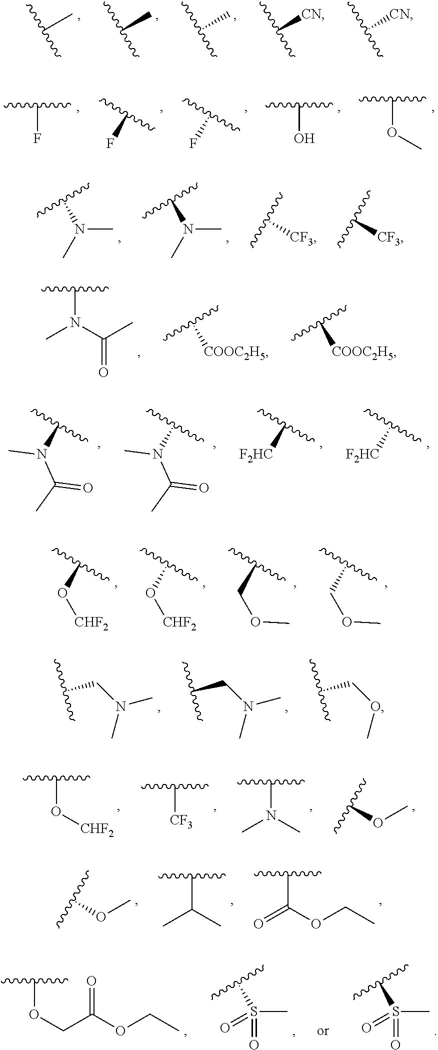

[13] The compound of any one of [1] to [12], wherein,
the moiety of

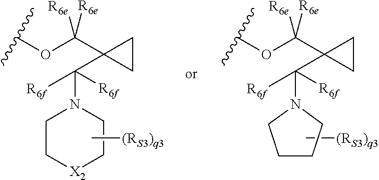

is selected from

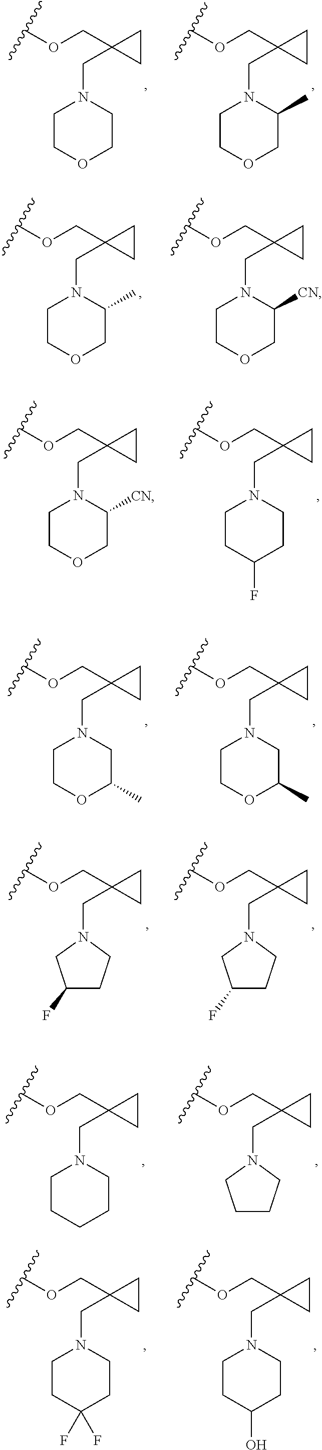

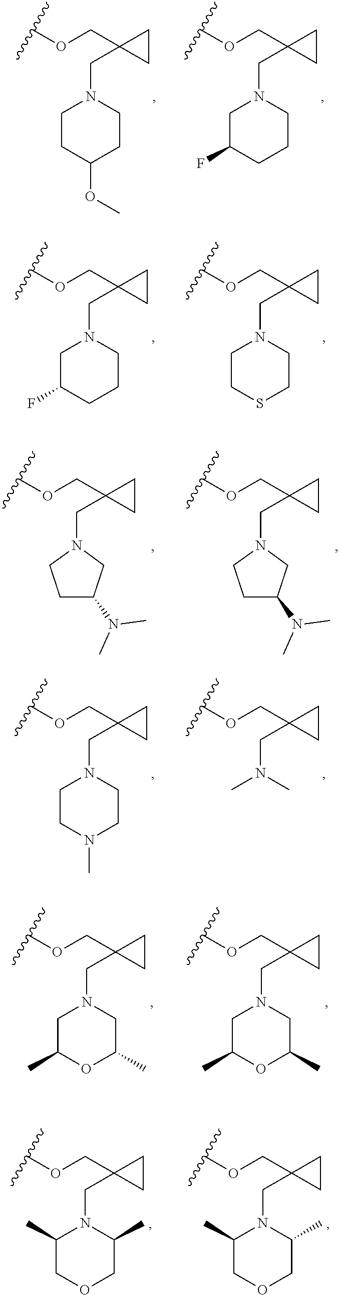

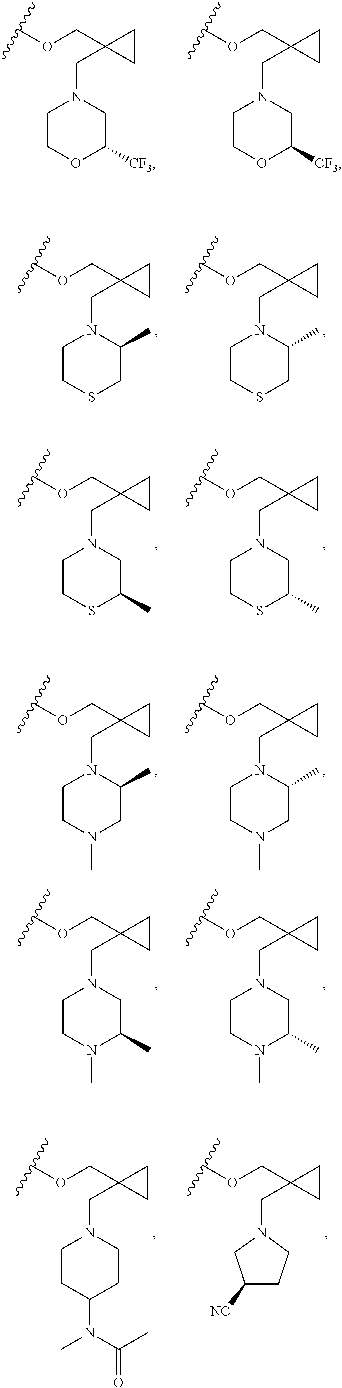

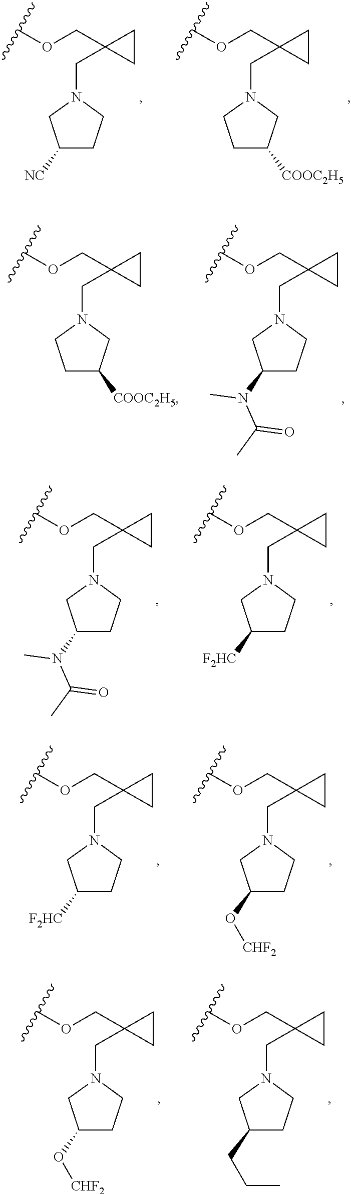

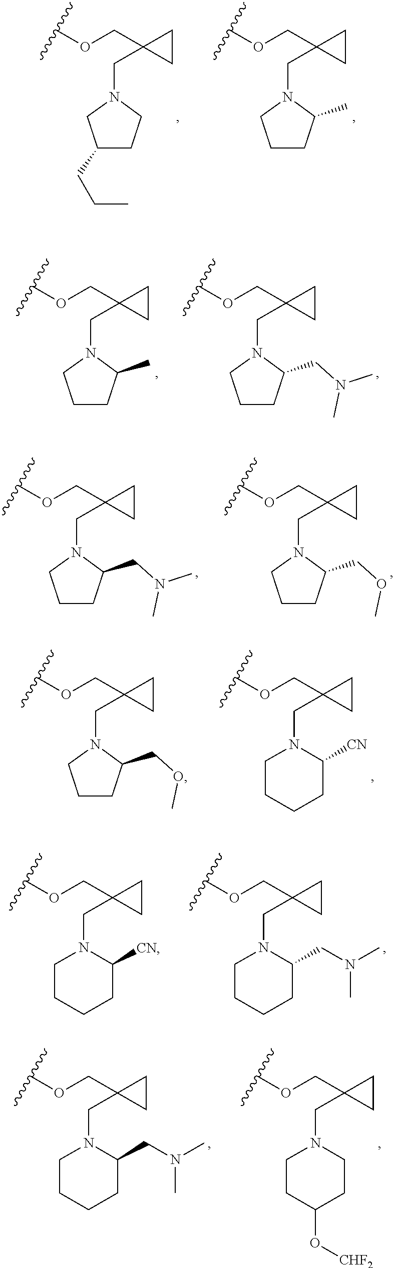

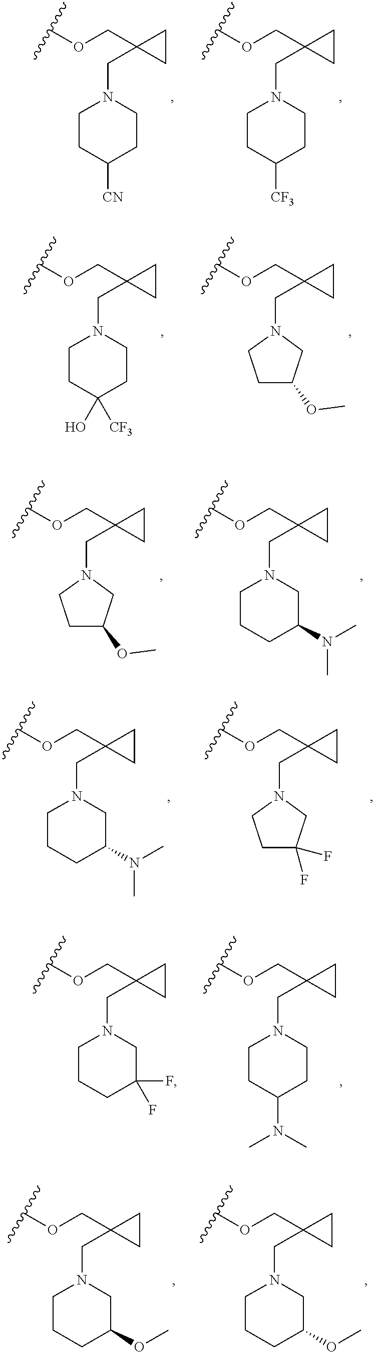

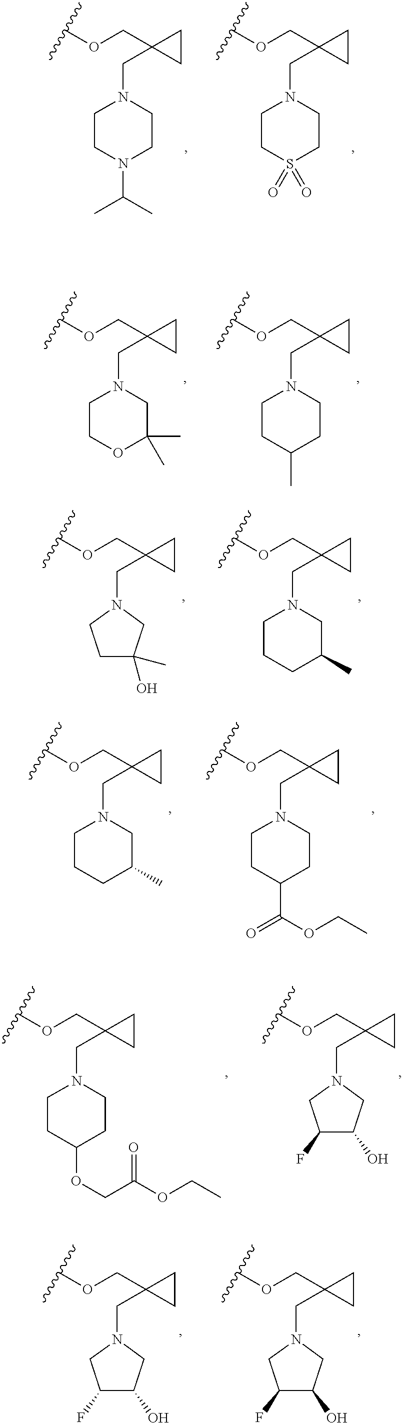

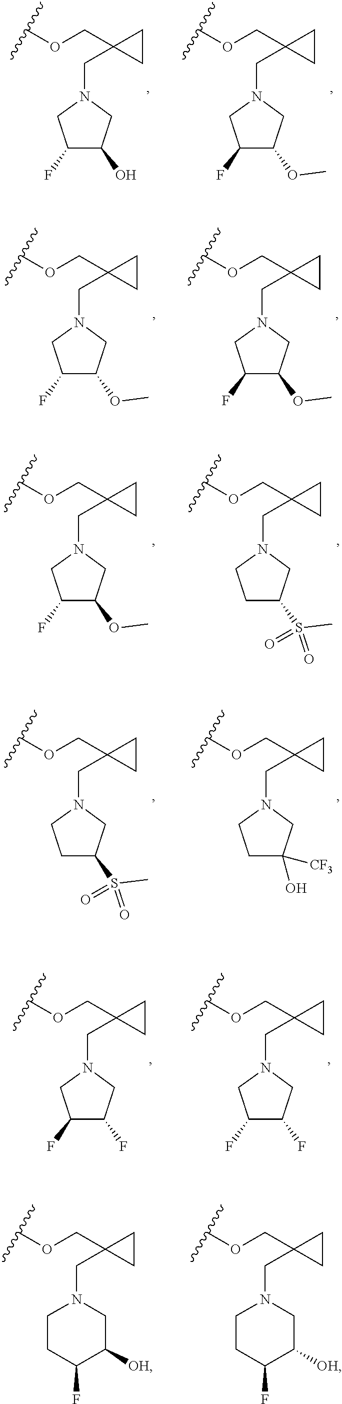

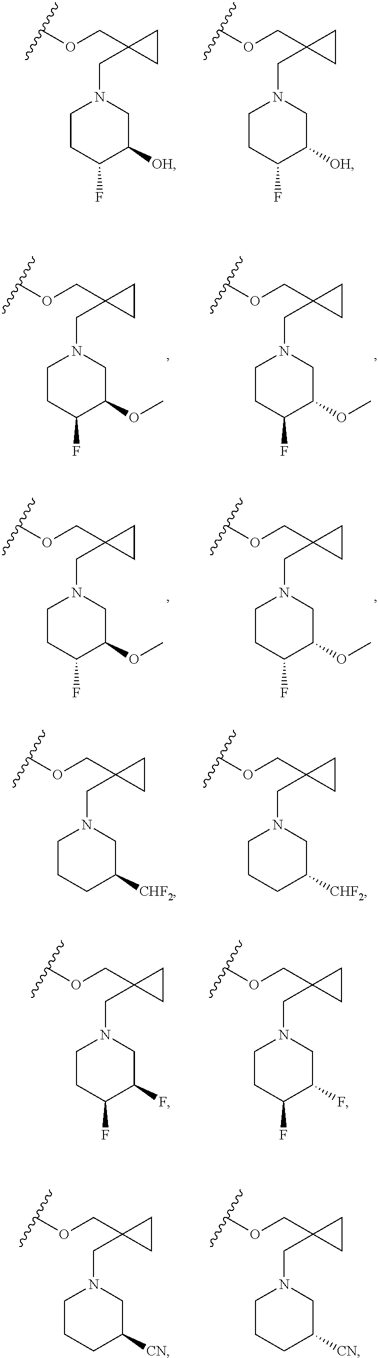

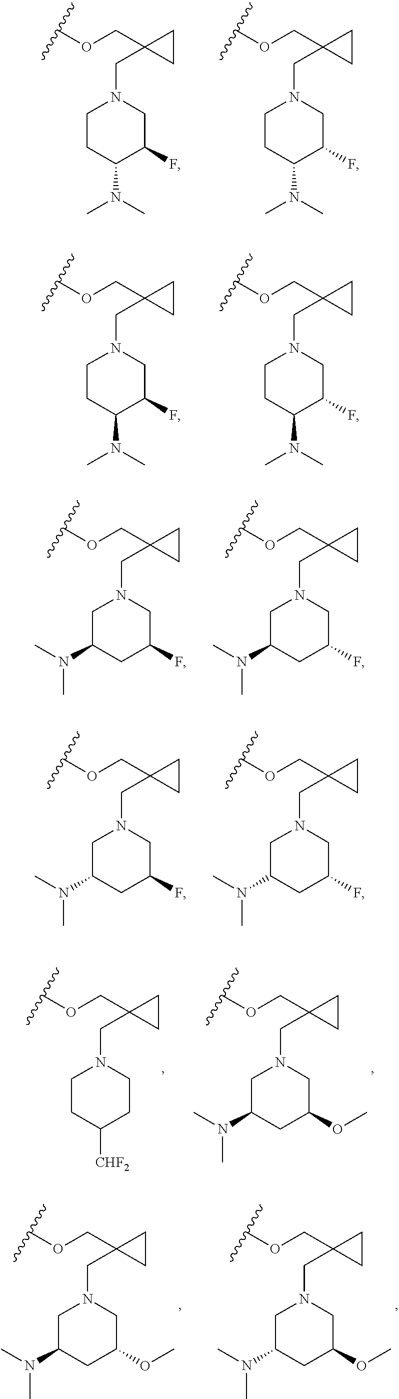

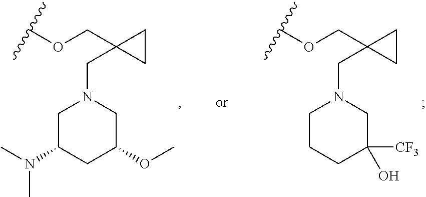

Preferably, the moiety of

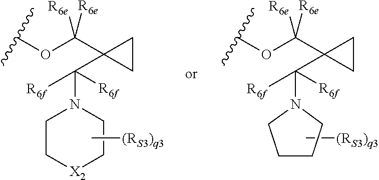

is selected from

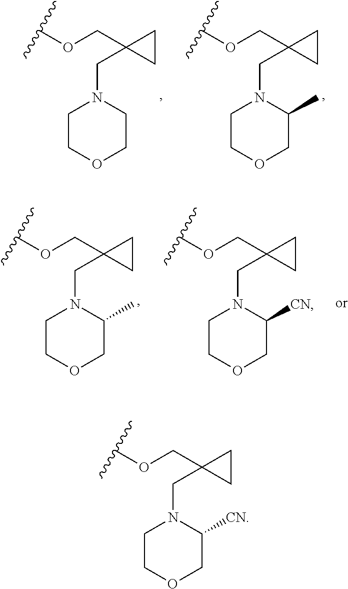

[14] The compound of any one of [1] to [13], wherein,

Each of (R₂ and R₅) at each occurrence is independently selected from halogen, —C₁₋₆alkyl, haloC₁₋₆alkyl, haloC₁₋₆alkoxy, —C₂₋₆alkenyl, —C₂₋₆alkynyl, —CN, —N(R₈₁)₂, —OR₈₁, —SR₈₁, —S(═O)R₈₂, —S(═O)₂R₈₂, —C(═O)R₈₂, —C(═O)OR₈₁, —OC(═O)R₈₂, —P(═O)(R₈₂)₂, 3-10 membered cycloalkyl, or 3-10 membered heterocyclyl, wherein said —C₁₋₆alkyl, haloC₁₋₆alkyl, haloC₁₋₆alkoxy, —C₂₋₆alkenyl, —C₂₋₆alkynyl, 3-10 membered cycloalkyl, or 3-10 membered heterocyclyl is optionally independently substituted with 1, 2, or 3 substituents selected from halogen, —C₁₋₃alkyl, —C₂₋₃alkenyl, —C₂₋₃alkynyl, —CN, —N(R₈₃)₂, —OR₈₃, —SR₈₃, —S(═O)R₈₄, —S(═O)₂R₈₄, —C(═O)R₈₄, —C(═O)OR₈₃, —OC(═O)R₈₃, —P(═O)(R₈₄)₂, 3-6 membered cycloalkyl, or 3-6 membered heterocyclyl;

Each of (R₈₁ and R₈₃) at each occurrence is independently selected from hydrogen, —C₁₋₆alkyl, —C₂₋₆alkenyl, or —C₂₋₆alkynyl, wherein said —C₁₋₆alkyl, —C₂₋₆alkenyl, or —C₂₋₆alkynyl is optionally independently substituted with 1, 2, or 3 substituents selected from halogen;

Each of (R₈₂ and R₈₄) at each occurrence is independently selected from hydrogen, —C₁₋₃alkyl, —C₂₋₃alkenyl, or —C₂₋₃alkynyl, wherein said —C₁₋₆alkyl, —C₂₋₆alkenyl, or —C₂₋₆alkynyl is optionally independently substituted with 1, 2, or 3 substituents selected from halogen;

Each of R_{S1} at each occurrence is independently selected from halogen, —C₁₋₆alkyl, haloC₁₋₆alkyl, haloC₁₋₆alkoxy, —C₂₋₆alkenyl, —C₂₋₆alkynyl, —CN, —N(R₆₁)₂, —OR₆₁, —SR₆₁, —S(═O)R₆₂, —S(═O)₂R₆₂, —C(═O)R₆₂, —C(═O)OR₆₁, —OC(═O)R₆₂, 3-6 membered cycloalkyl, or 3-6 membered heterocyclyl; wherein, said —C₁₋₆alkyl, haloC₁₋₆alkyl, haloC₁₋₆alkoxy, —C₂₋₆alkenyl, —C₂₋₆alkynyl, 3-6 membered cycloalkyl, or 3-6 membered heterocyclyl is optionally independently substituted with 1, 2, or 3 substituents selected from halogen, —C₁₋₃alkyl, haloC₁₋₃alkyl, haloC₁₋₃alkoxy, —C₂₋₃alkenyl, —C₂₋₃alkynyl, —CN, —N(R₆₃)₂, —OR₆₃, —SR₆₃, —S(═O)R₆₄, —S(═O)₂R₆₄, —C(═O)R₆₄, —C(═O)OR₆₃, —OC(═O)R₆₄, 3-6 membered cycloalkyl, or 3-6 membered heterocyclyl;

Each of (R₆₁ and R₆₃) at each occurrence is independently selected from hydrogen, —C₁₋₆alkyl, —C₂₋₆alkenyl, or —C₂₋₆alkynyl;

Each of (R₆₂ and R₆₄) at each occurrence is independently selected from hydrogen, —C₁₋₃alkyl, —C₂₋₃alkenyl, or —C₂₋₃alkynyl;

Each of q₁ is independently selected from 0, 1, 2, or 3;

Preferably, R₂ is selected from

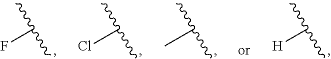

R_{S} is hydrogen;
q₁ is 0.

[15] The compound of any one of [1] to [14], wherein,
the compound is of formula (V-1):

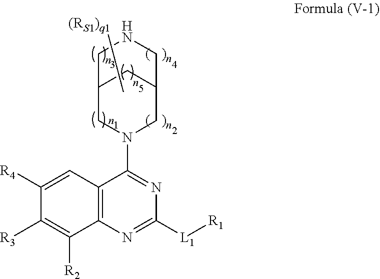

Wherein,
moiety of

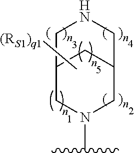

is selected from

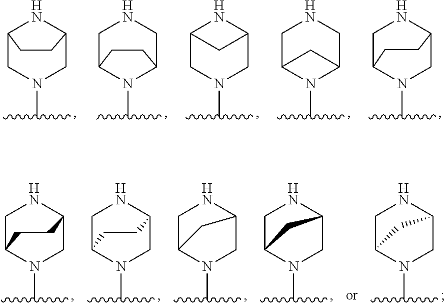

-L₁-R₁ is selected from

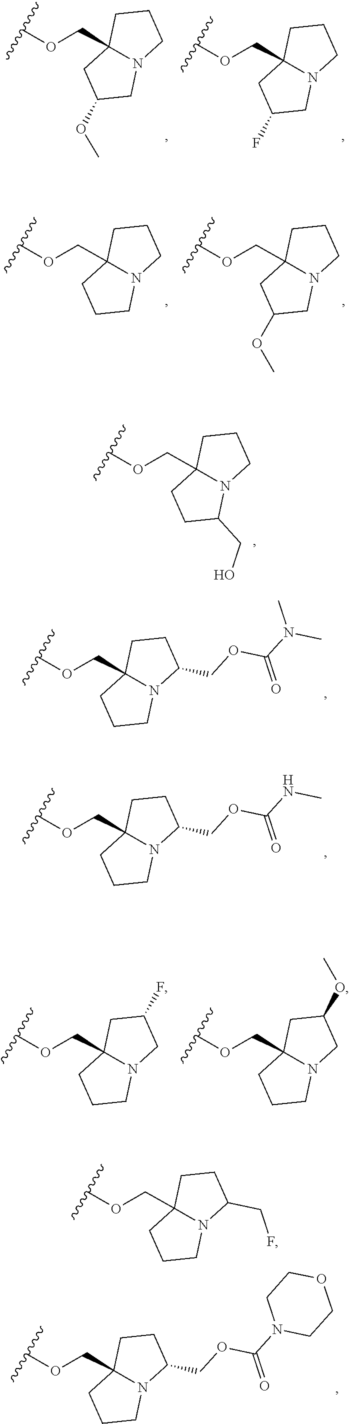

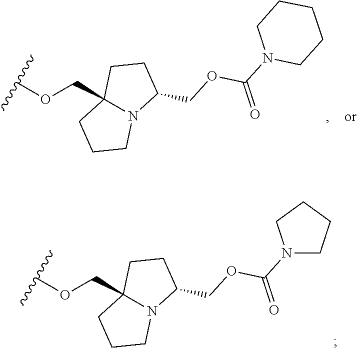 [SMILES: *OC[C@]12CCCN1[C@@H](COC(=O)N1CCCCC1)CC2 ; *OC[C@]12CCCN1[C@@H](COC(=O)N1CCCC1)CC2]

R₂ is selected from

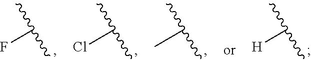

R₃ is selected from

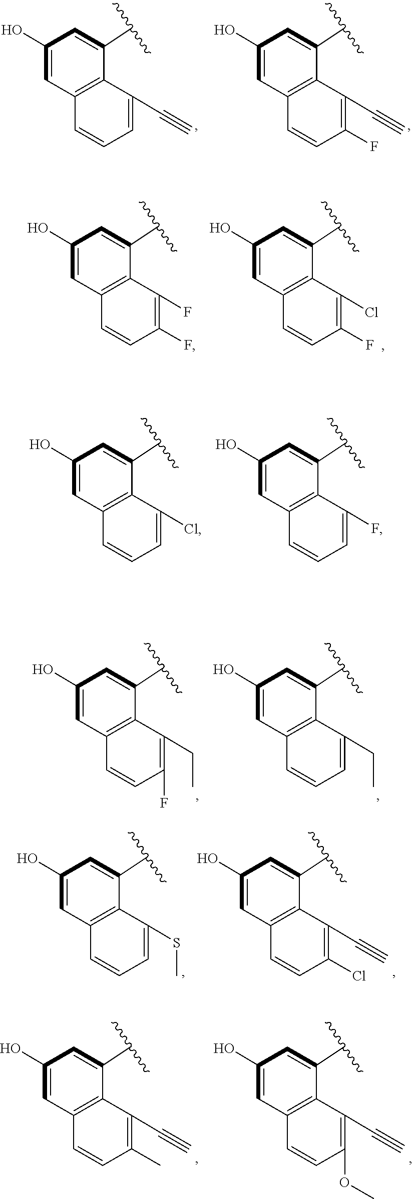

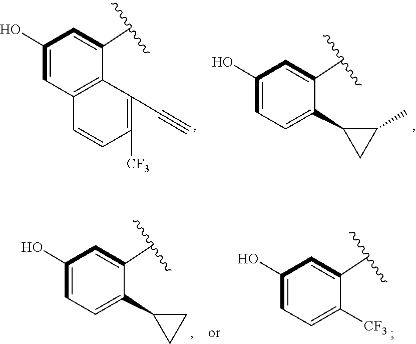

and
R₄ is selected from

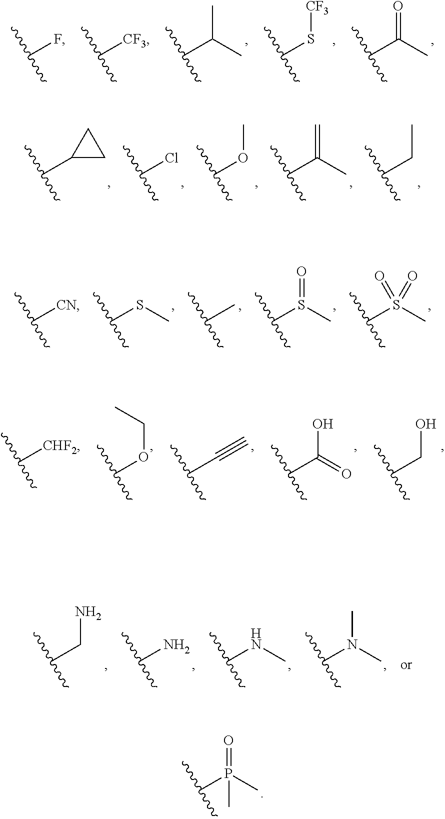

[16] The compound of any one of [1] to [14], wherein,
the compound is of formula (V-2):

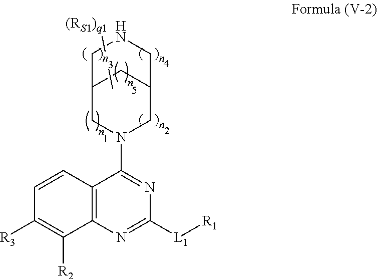

Wherein
moiety of

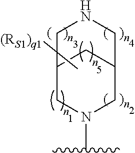

is selected from

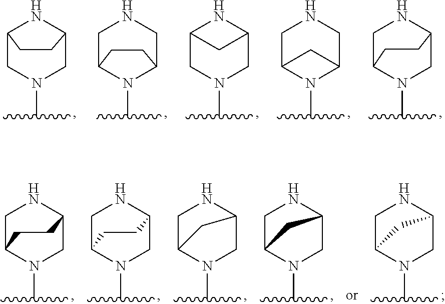

-L₁-R₁ is selected from

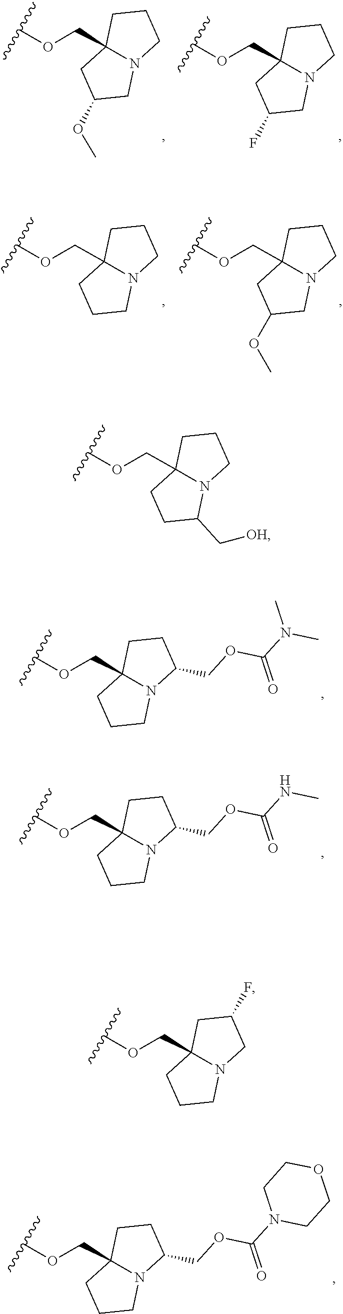

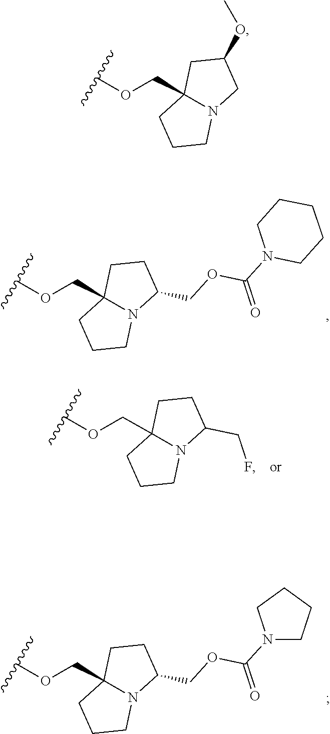 [SMILES: *OC[C@]12CCCN1C[C@H](OC)C2 ; *OC[C@]12CCCN1[C@@H](COC(=O)N1CCCCC1)CC2 ; *OCC12CCCN1C(CF)CC2 ; *OC[C@]12CCCN1[C@@H](COC(=O)N1CCCC1)CC2]

R₂ is selected from

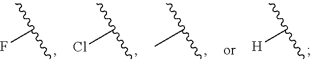

and
R₃ is selected from

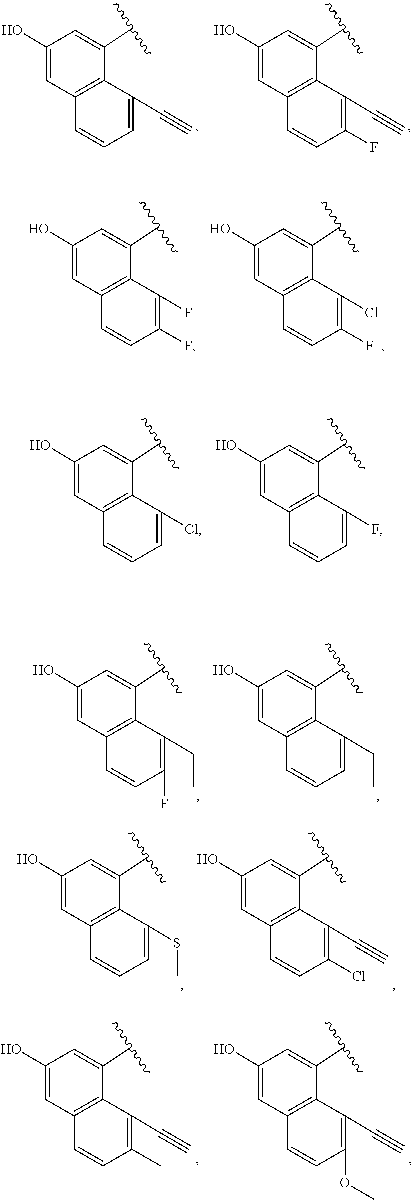

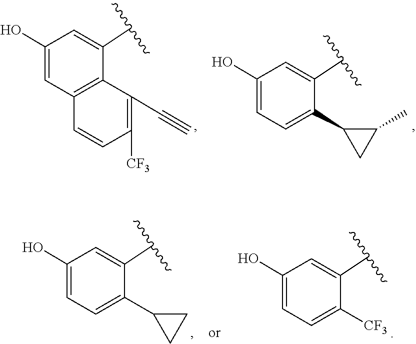 [SMILES: *c1cc(O)cc2ccc(C(F)(F)F)c(C#C)c12 ; *c1cc(O)ccc1[C@@H]1C[C@H]1C ; *c1cc(O)ccc1C1CC1 ; *c1cc(O)ccc1C(F)(F)F]

[17] The compound of any one of [1] to [14], wherein,
the compound is of formula (V-3):

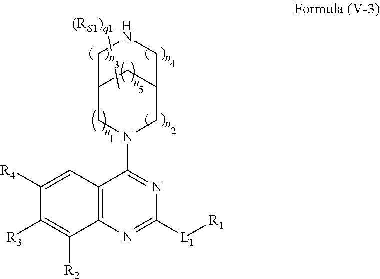

Wherein
moiety of

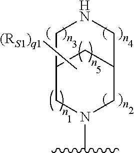

is selected from

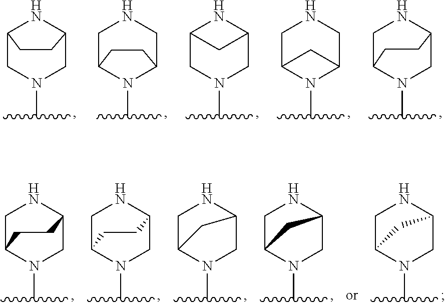

-L₁-R₁ is selected from

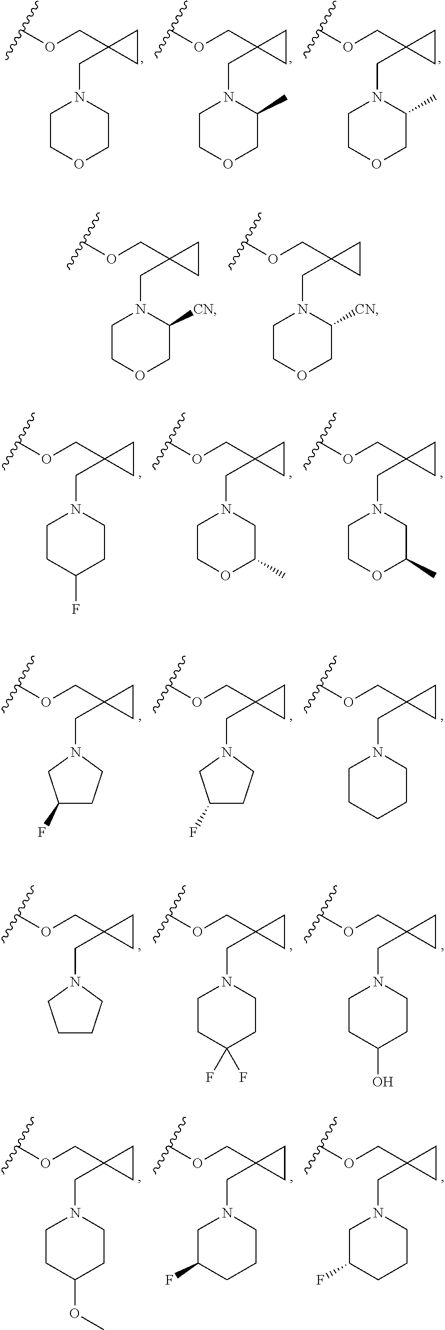

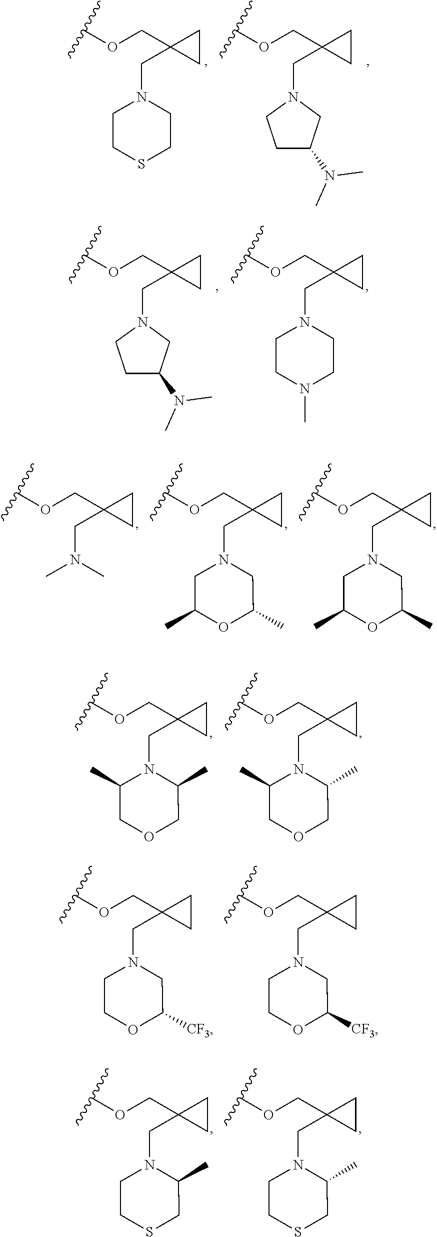

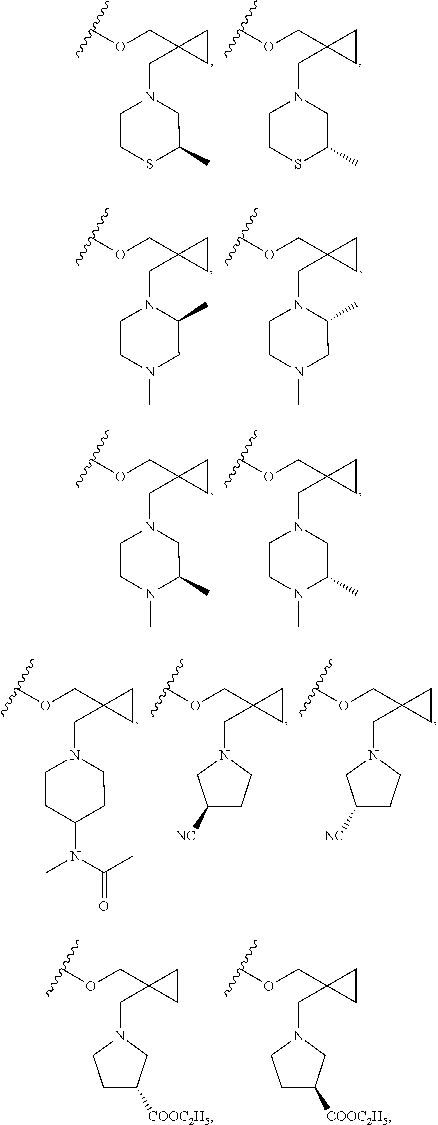

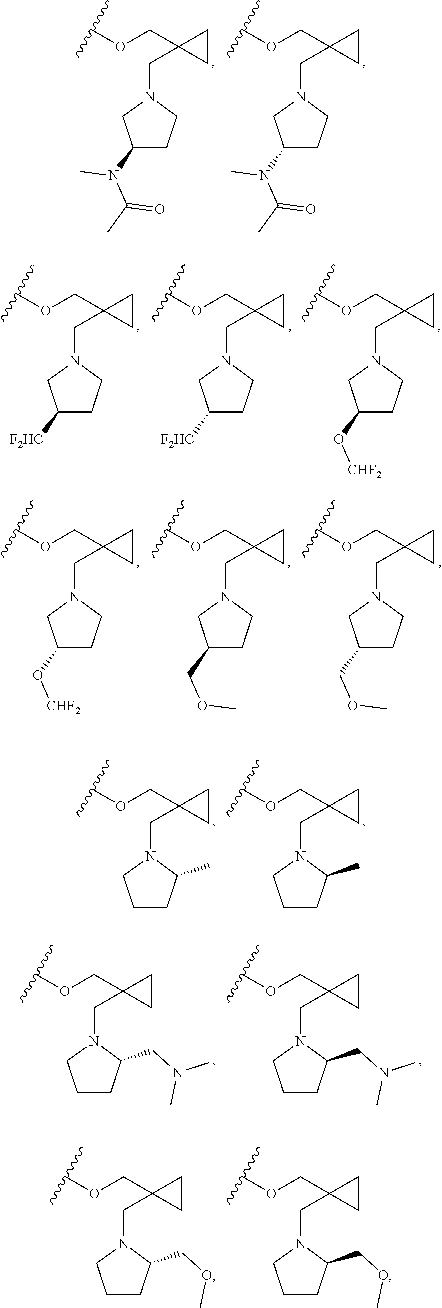

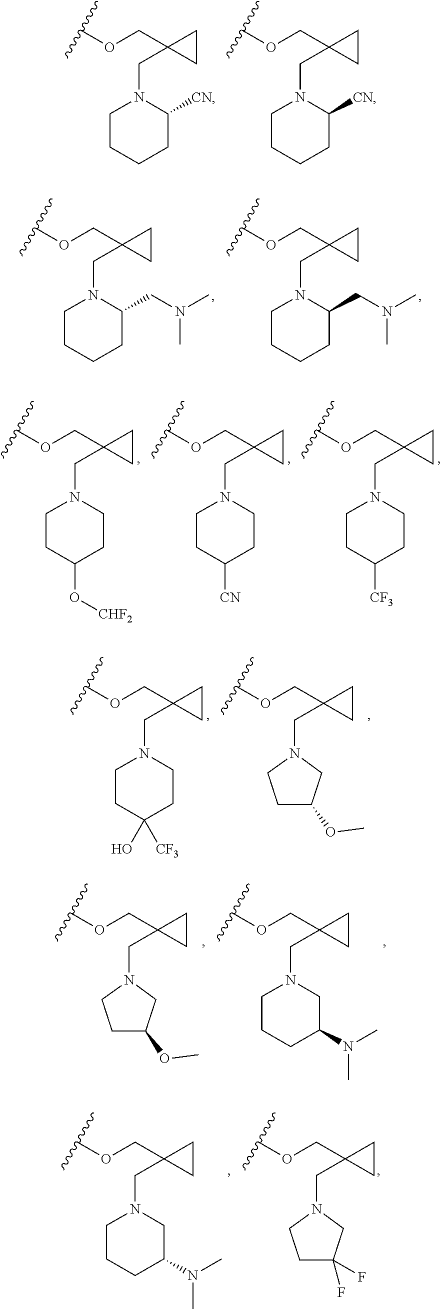

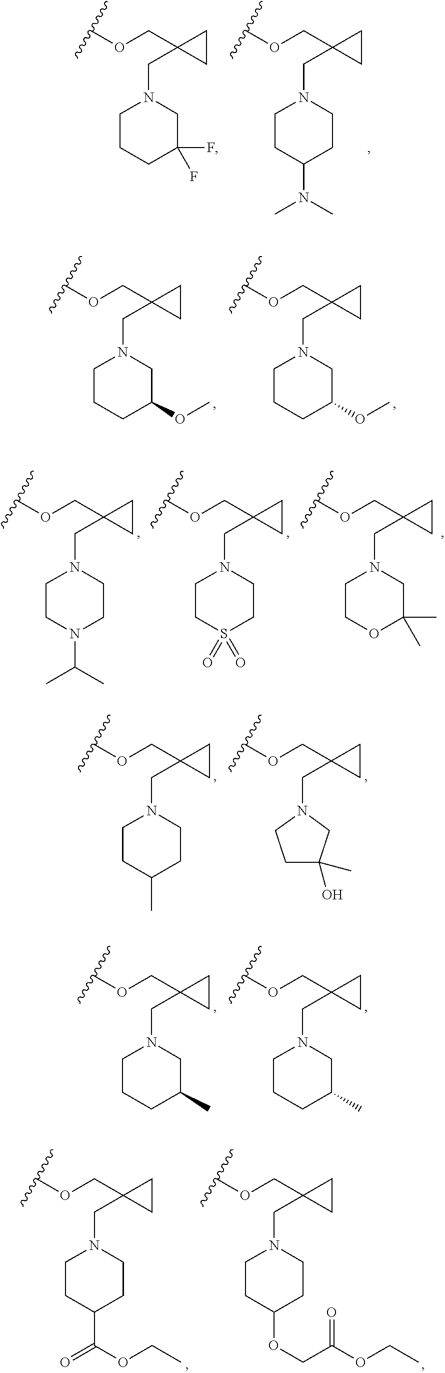

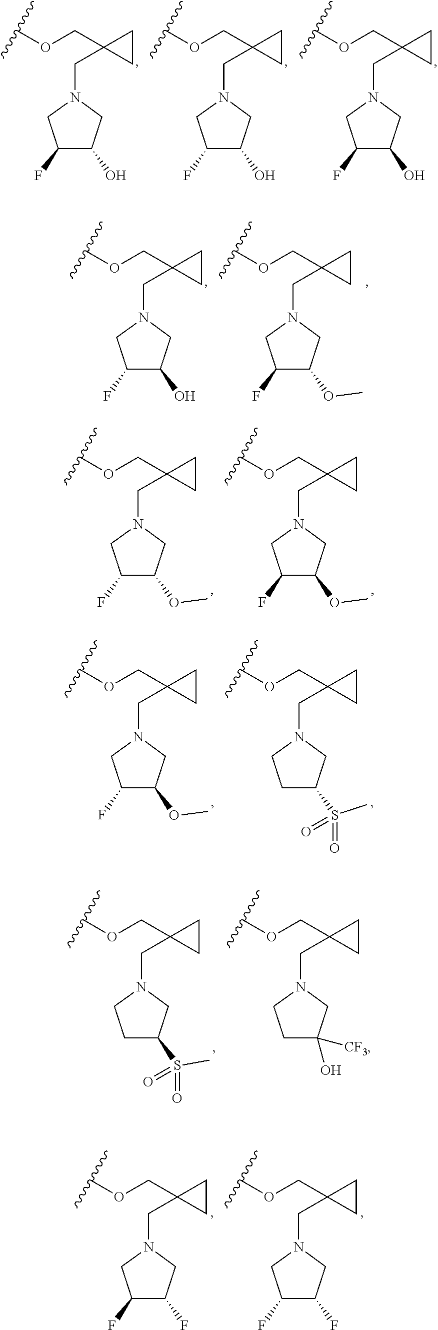

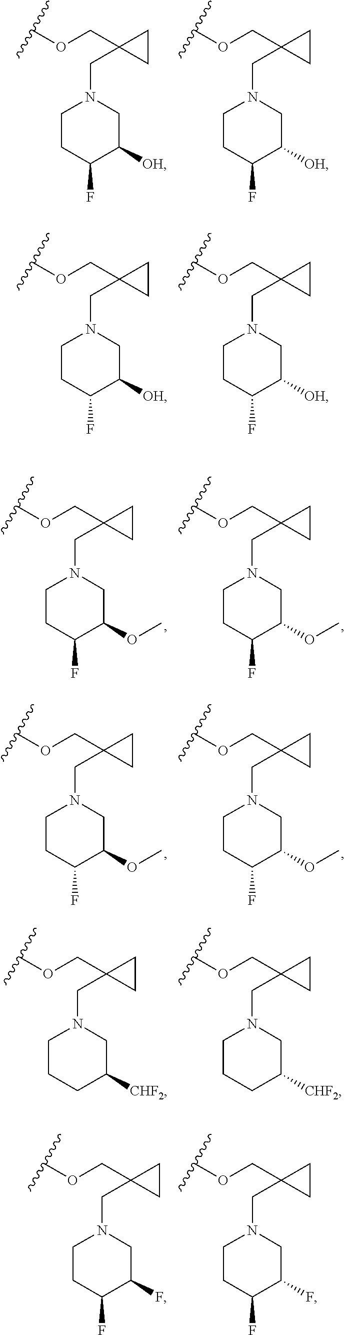

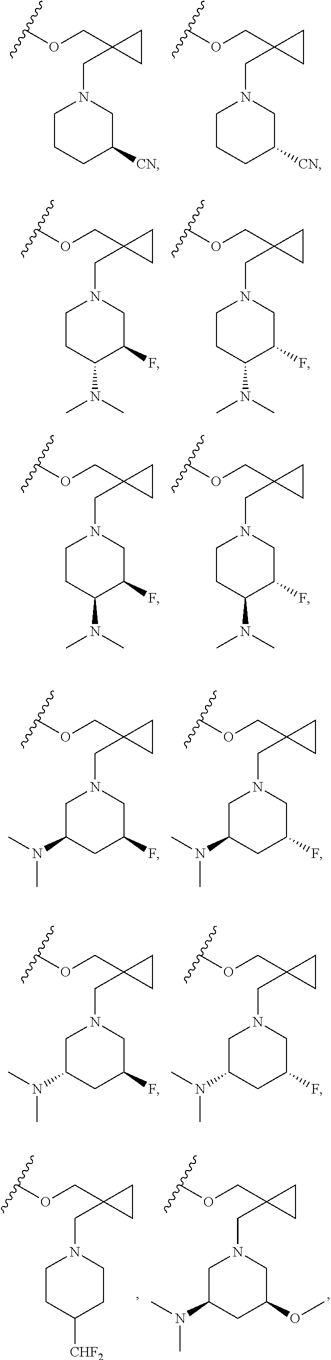

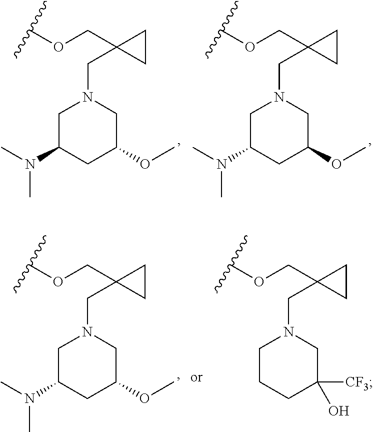

R₂ is selected from

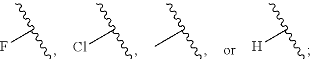

R₃ is selected from

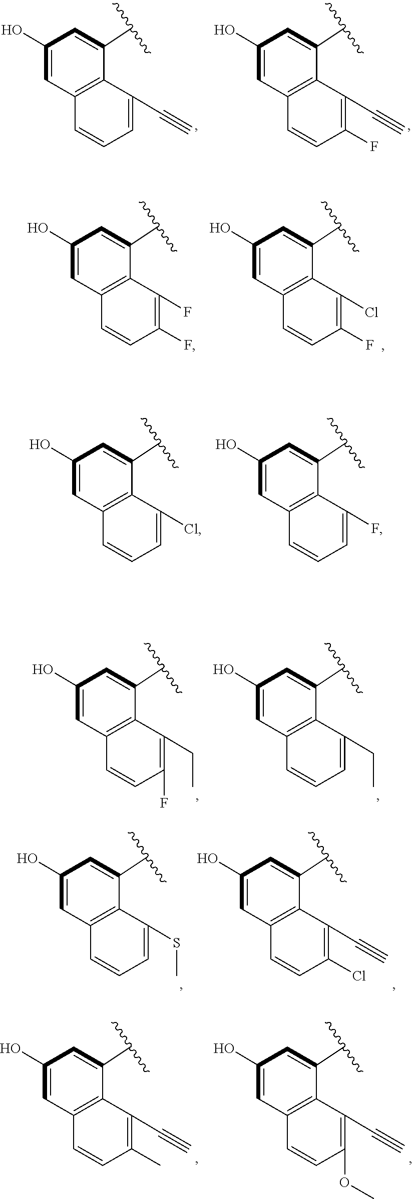

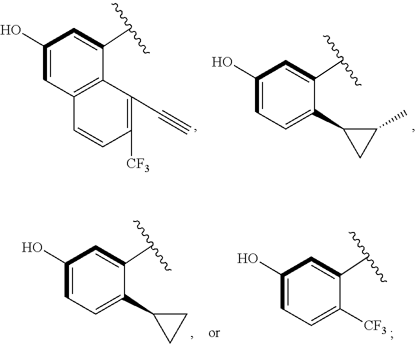

and
R₄ is selected from

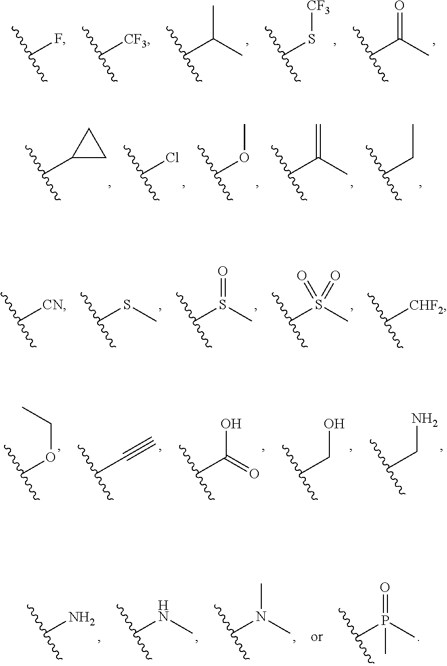

[18] The compound of any one of [1] to [14], wherein,
the compound is of formula (V-4):

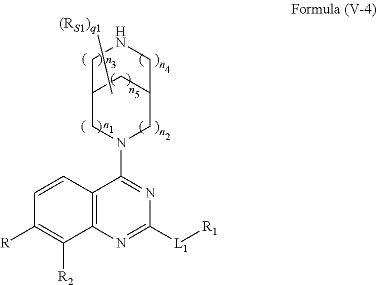

Wherein,
moiety of

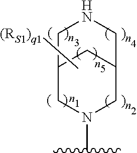

is selected from

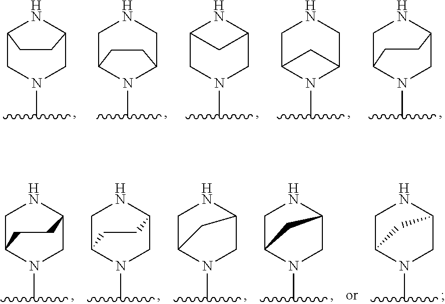

-L₁-R₁ is selected from

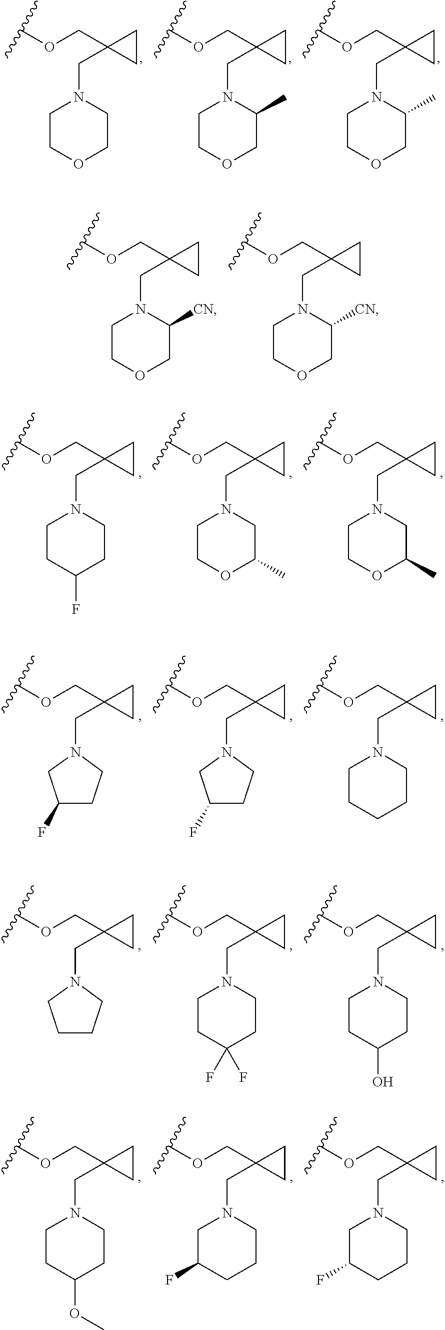

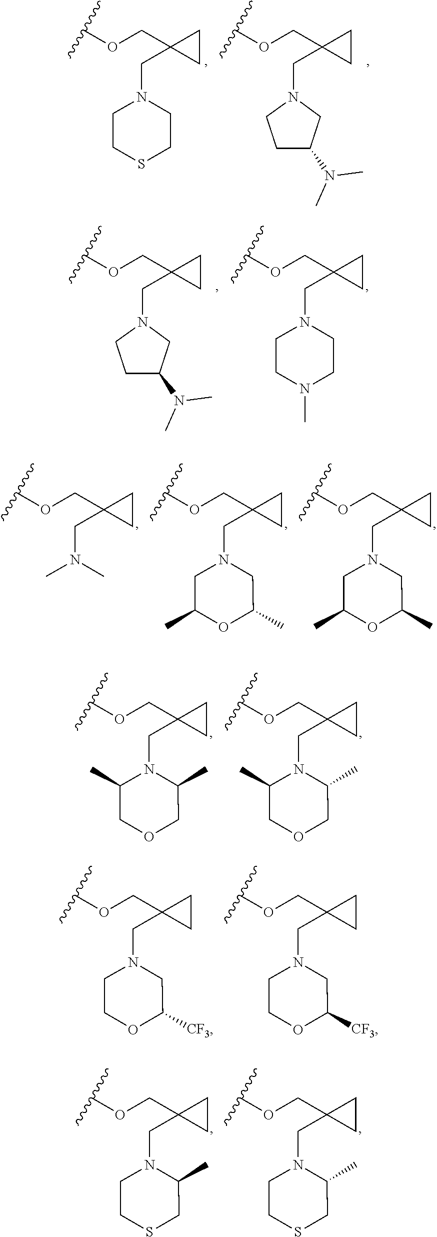

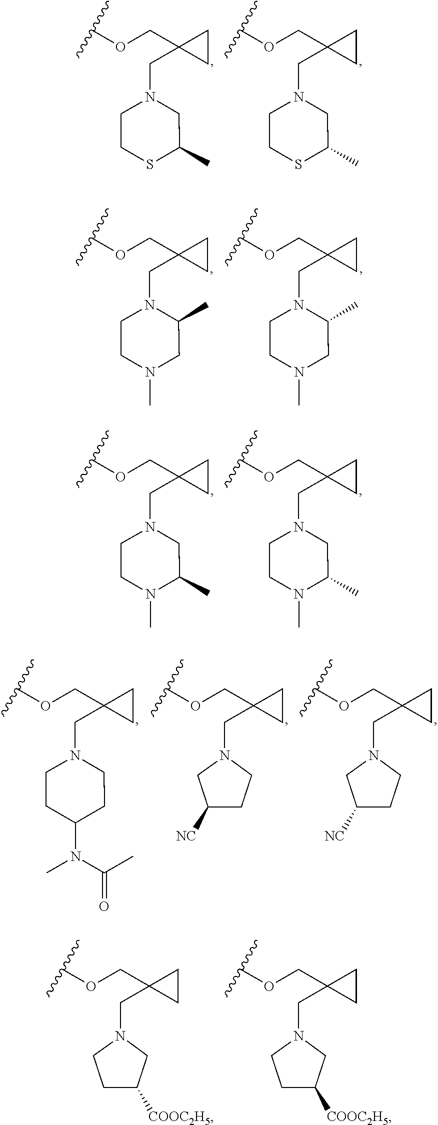

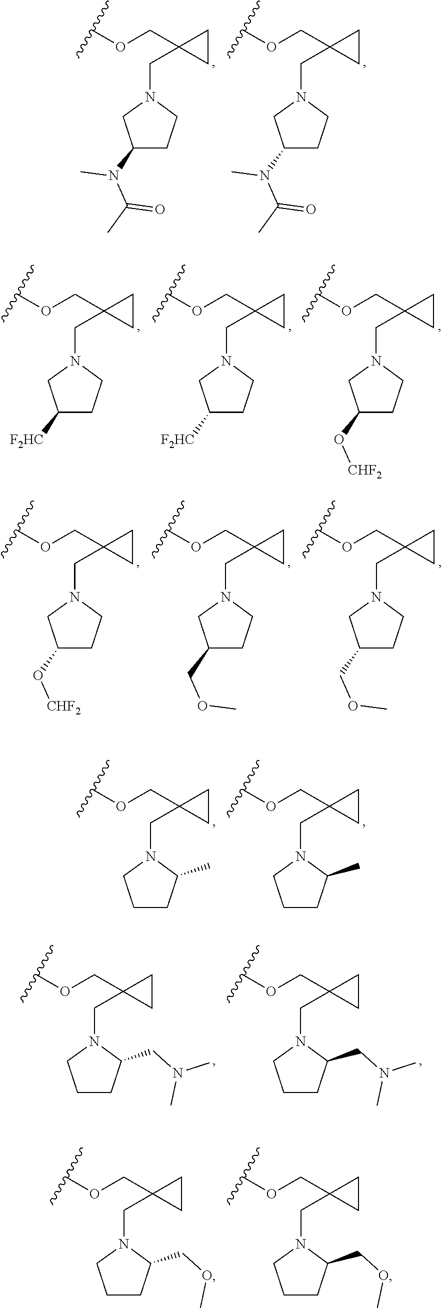

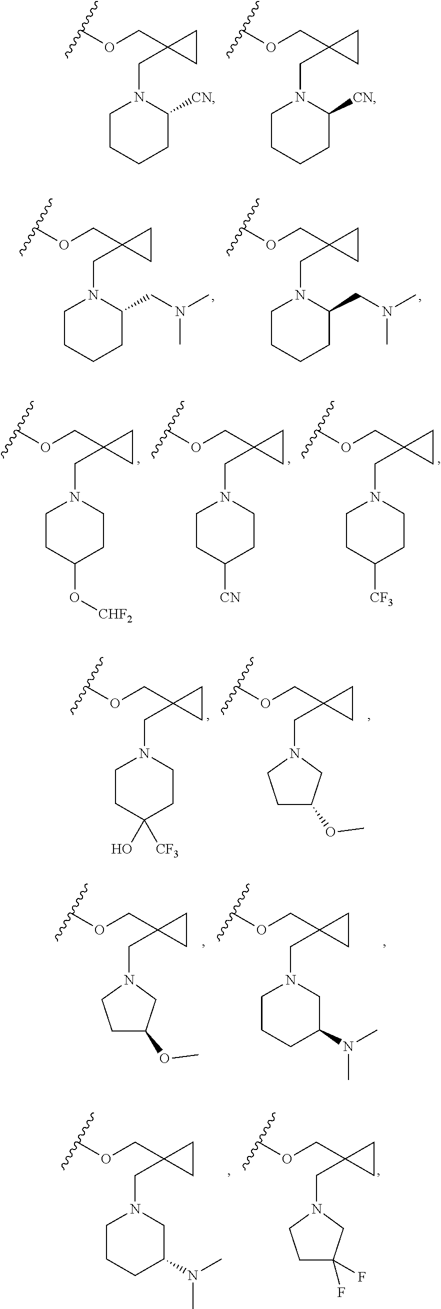

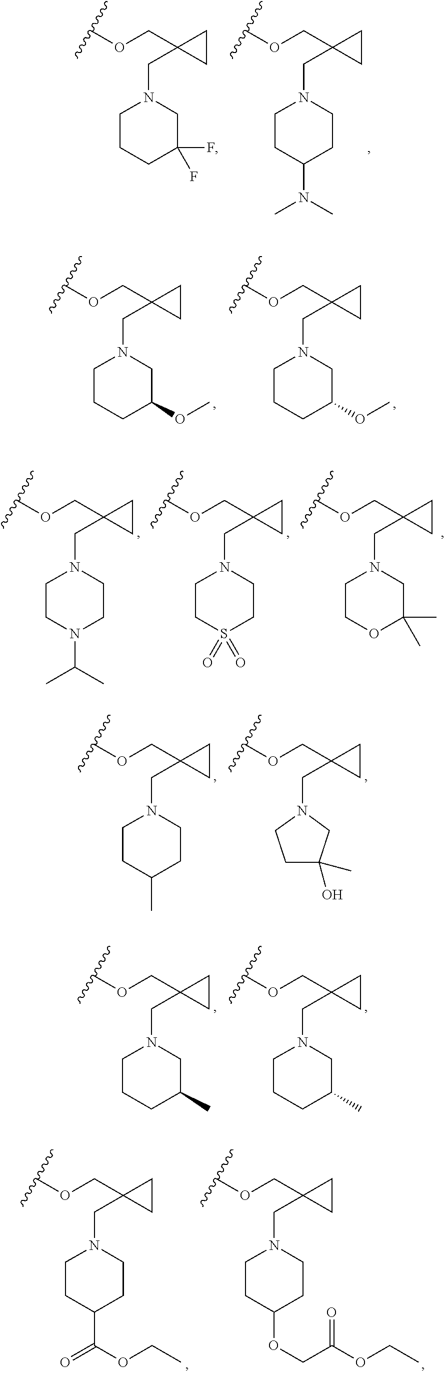

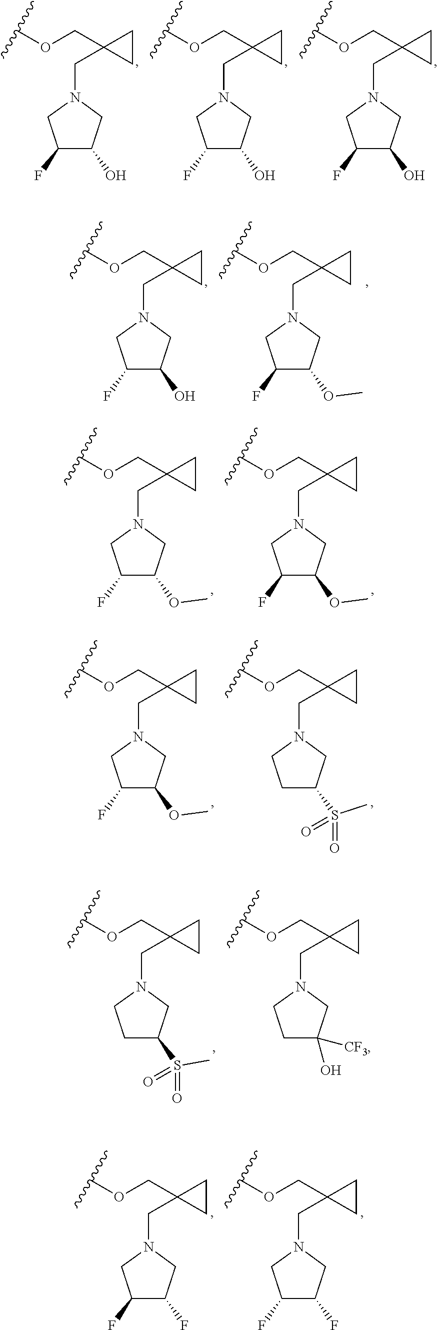

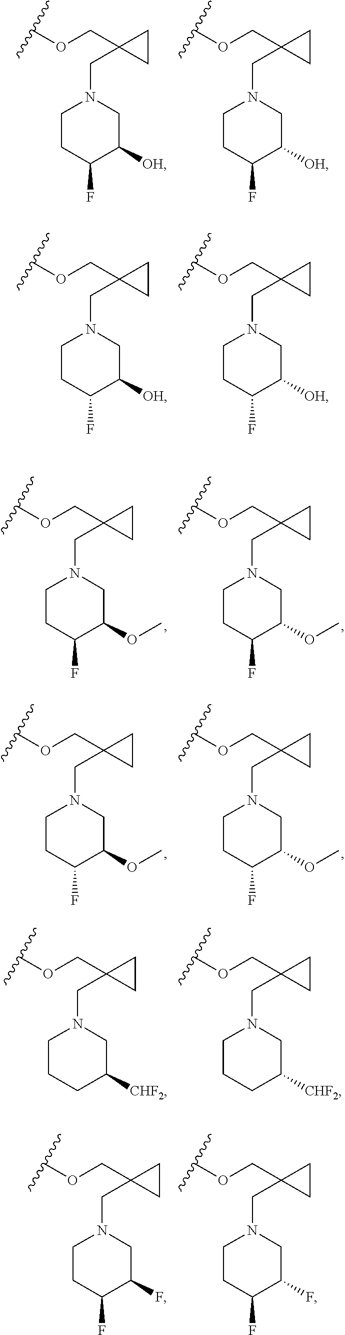

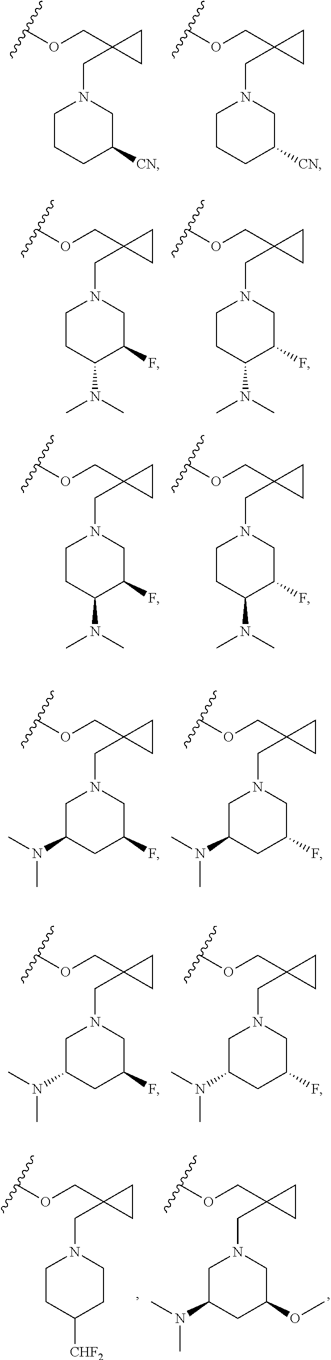

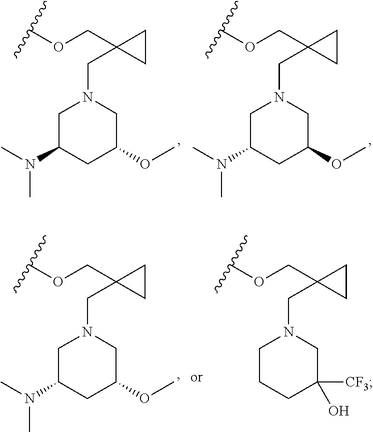

R₂ is selected from

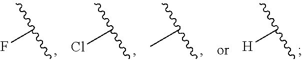

and
R₃ is selected from

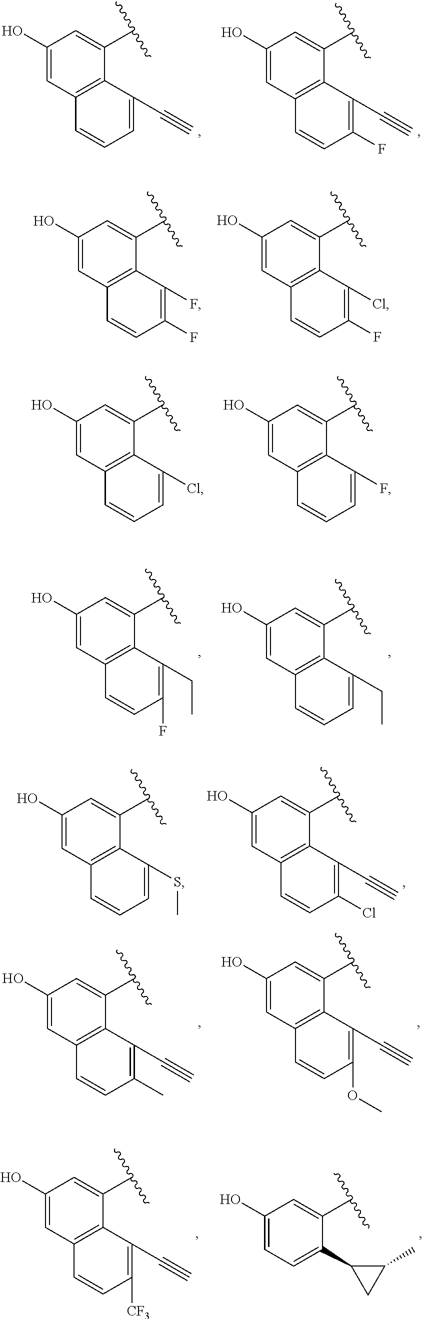

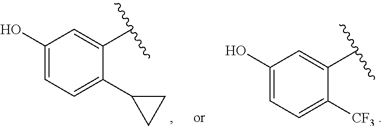 [SMILES: *c1cc(O)ccc1C1CC1 ; *c1cc(O)ccc1C(F)(F)F]

[19] The compound of any one of [1] to [14], wherein
the compound is selected from:

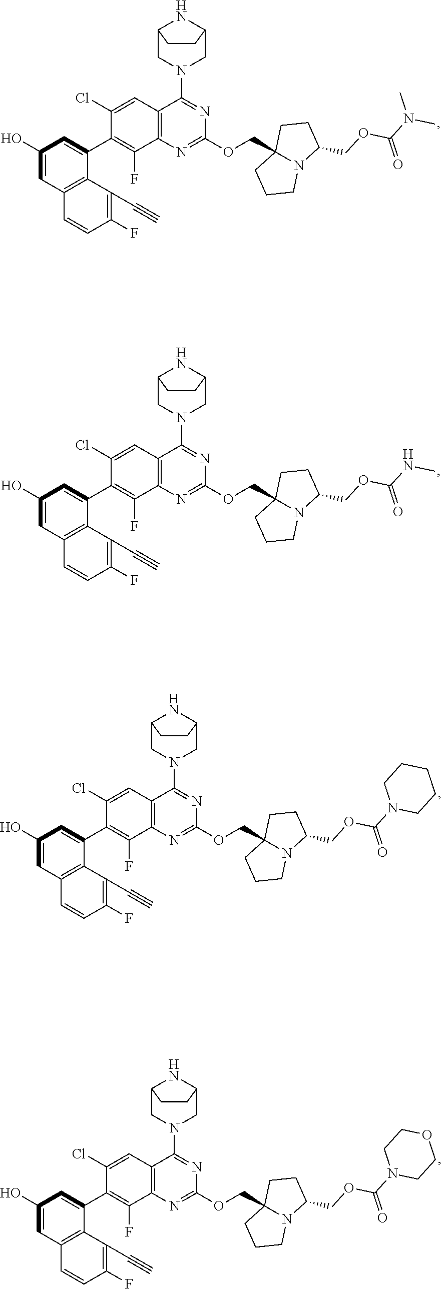

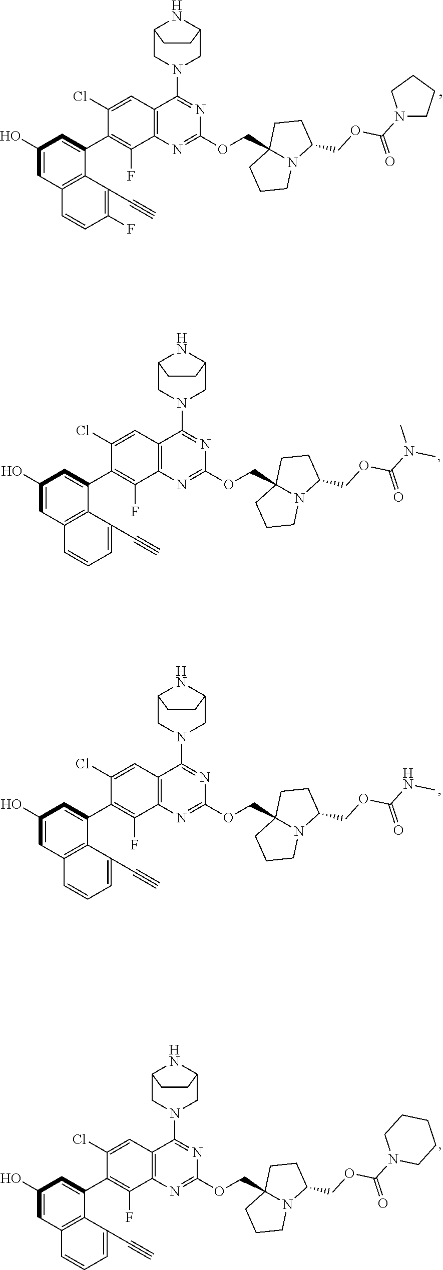

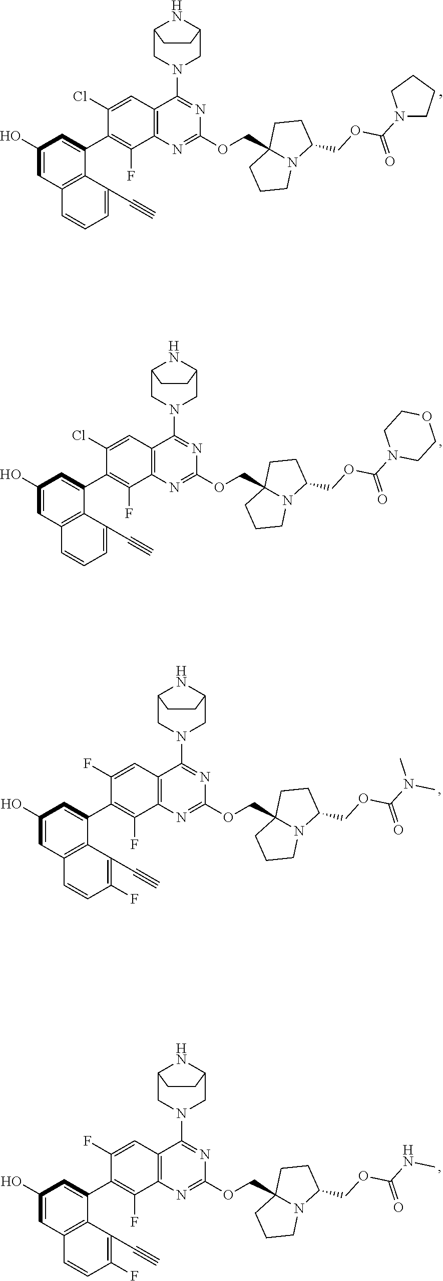

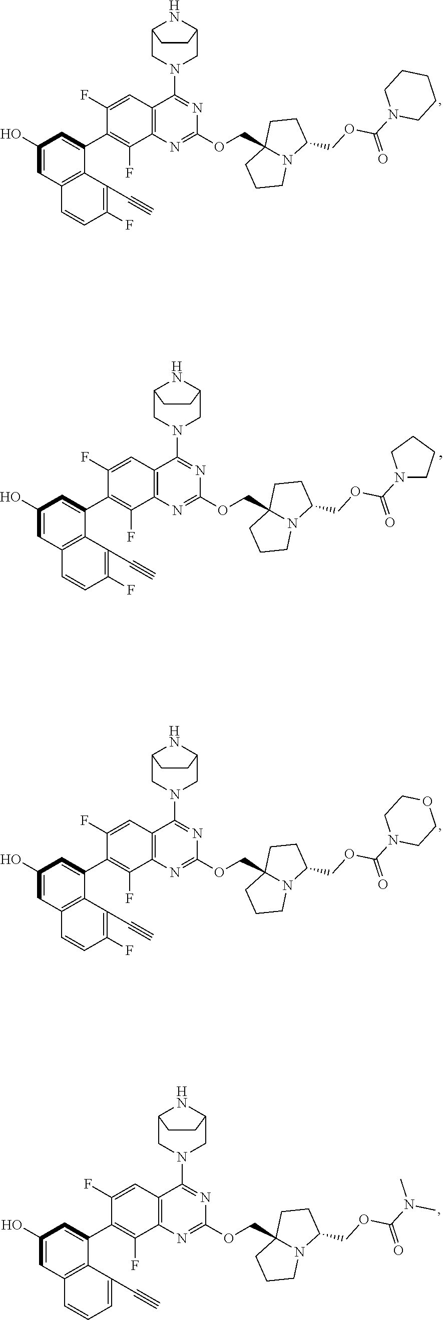

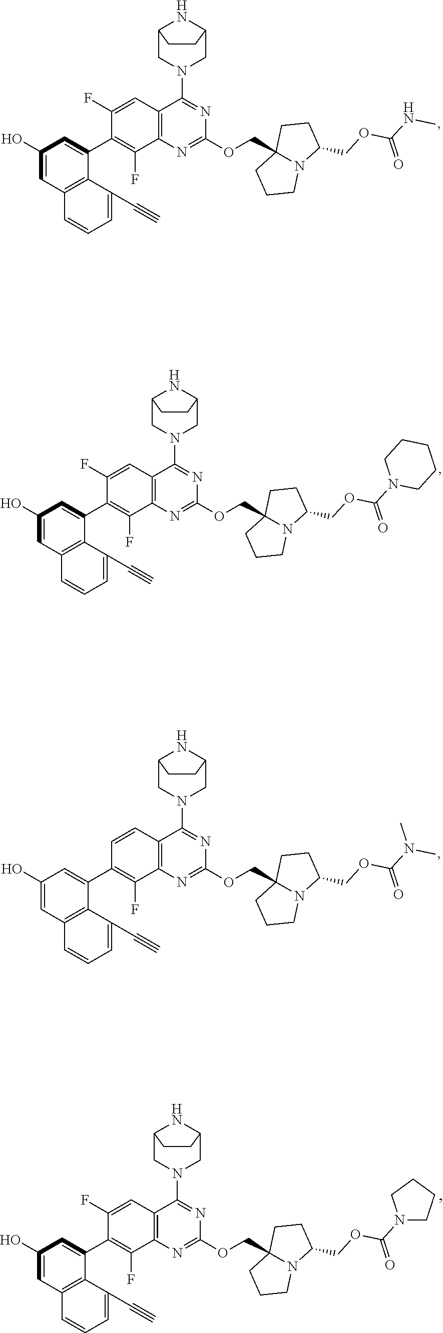

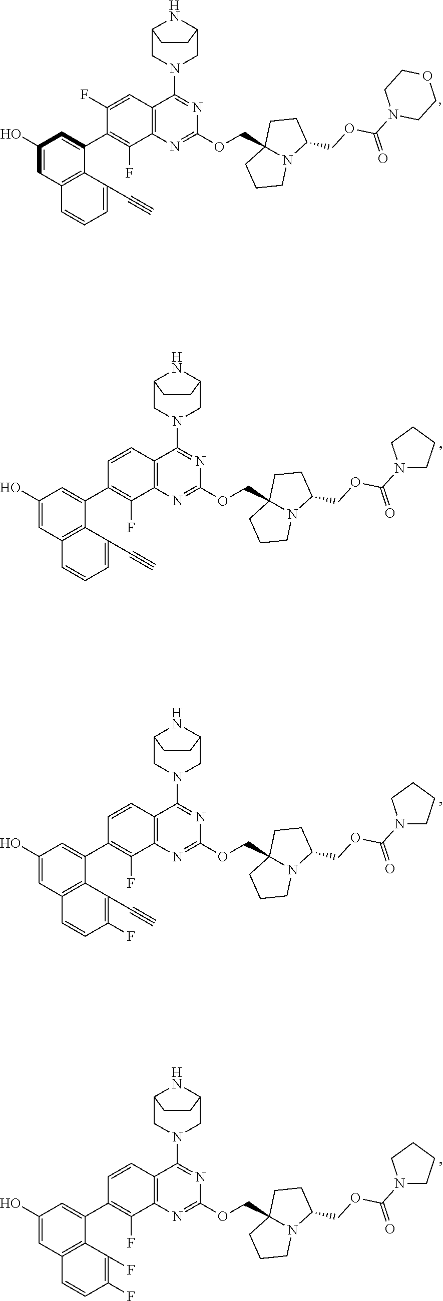

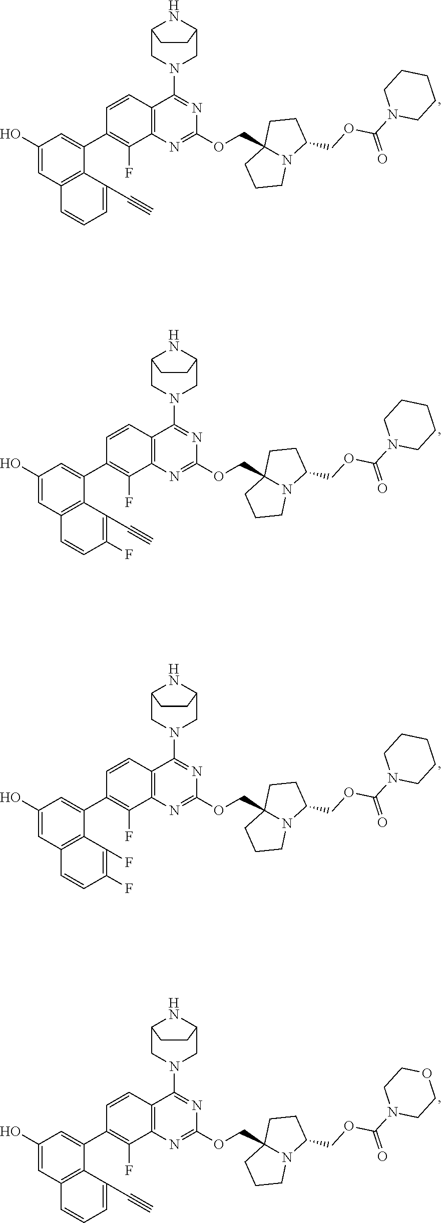 [SMILES: C#Cc1cccc2cc(O)cc(-c3ccc4c(N5CC6CCC(C5)N6)nc(OC[C@]56CCCN5[C@@H](COC(=O)N5CCCCC5)CC6)nc4c3F)c12 ; C#Cc1c(F)ccc2cc(O)cc(-c3ccc4c(N5CC6CCC(C5)N6)nc(OC[C@]56CCCN5[C@@H](COC(=O)N5CCCCC5)CC6)nc4c3F)c12 ; O=C(OC[C@H]1CC[C@@]2(COc3nc(N4CC5CCC(C4)N5)c4ccc(-c5cc(O)cc6ccc(F)c(F)c56)c(F)c4n3)CCCN12)N1CCCCC1 ; C#Cc1cccc2cc(O)cc(-c3ccc4c(N5CC6CCC(C5)N6)nc(OC[C@]56CCCN5[C@@H](COC(=O)N5CCOCC5)CC6)nc4c3F)c12]

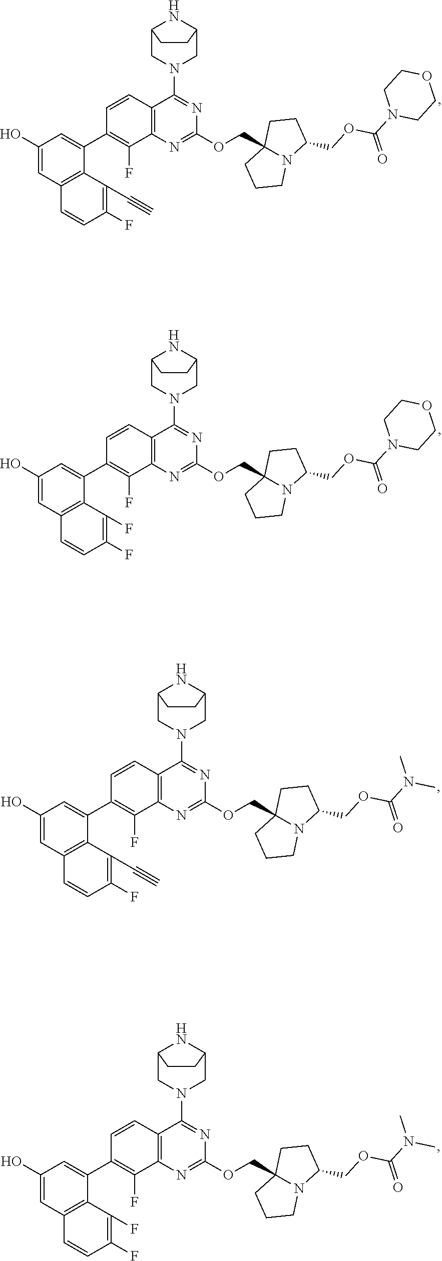 [SMILES: C#Cc1c(F)ccc2cc(O)cc(-c3ccc4c(N5CC6CCC(C5)N6)nc(OC[C@]56CCCN5[C@@H](COC(=O)N5CCOCC5)CC6)nc4c3F)c12 ; O=C(OC[C@H]1CC[C@@]2(COc3nc(N4CC5CCC(C4)N5)c4ccc(-c5cc(O)cc6ccc(F)c(F)c56)c(F)c4n3)CCCN12)N1CCOCC1 ; C#Cc1c(F)ccc2cc(O)cc(-c3ccc4c(N5CC6CCC(C5)N6)nc(OC[C@]56CCCN5[C@@H](COC(=O)N(C)C)CC6)nc4c3F)c12 ; CN(C)C(=O)OC[C@H]1CC[C@@]2(COc3nc(N4CC5CCC(C4)N5)c4ccc(-c5cc(O)cc6ccc(F)c(F)c56)c(F)c4n3)CCCN12]

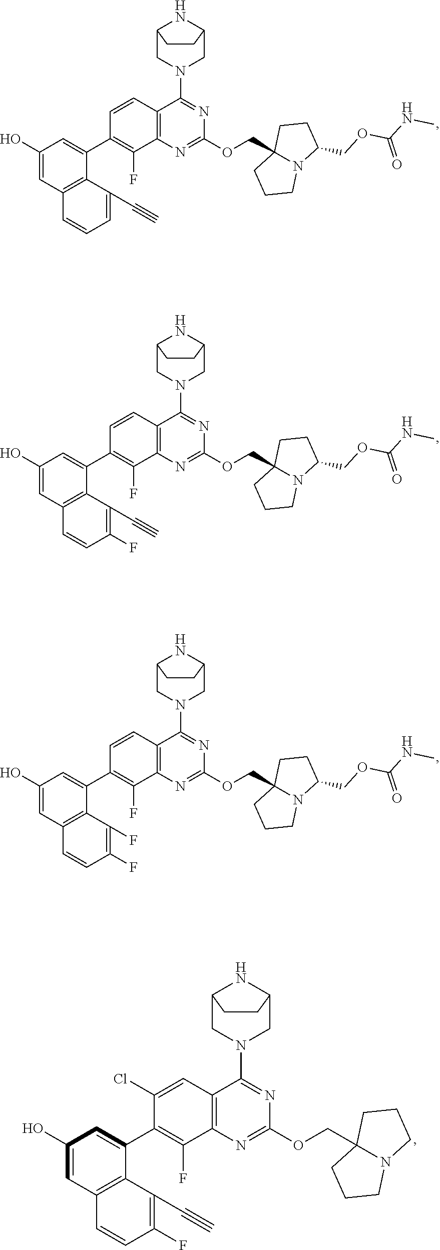

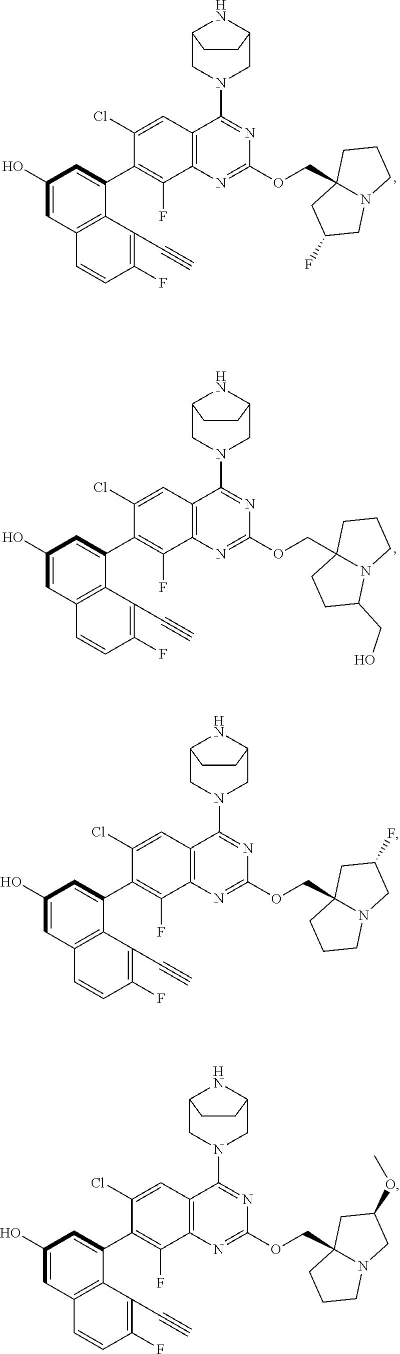

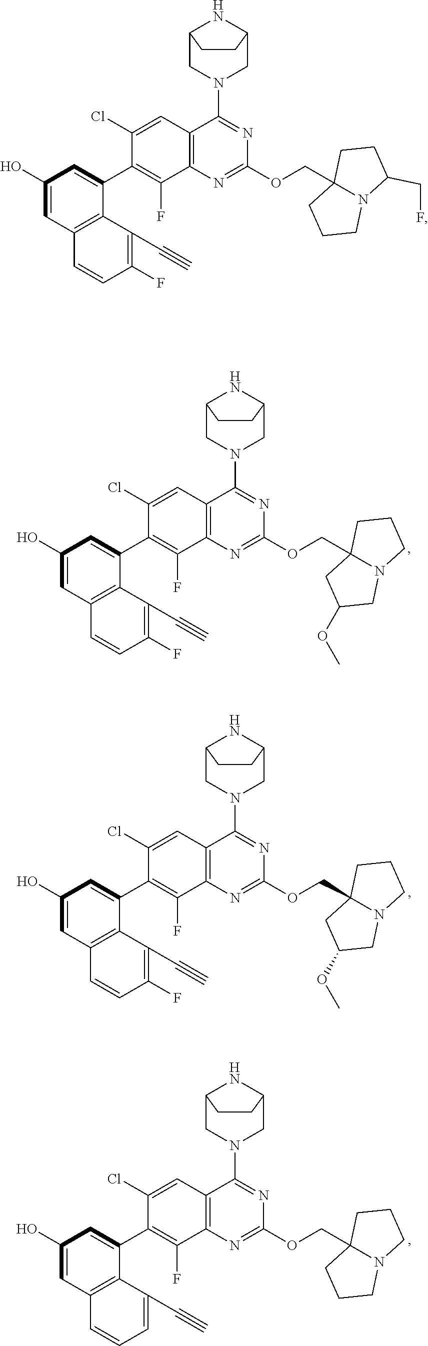

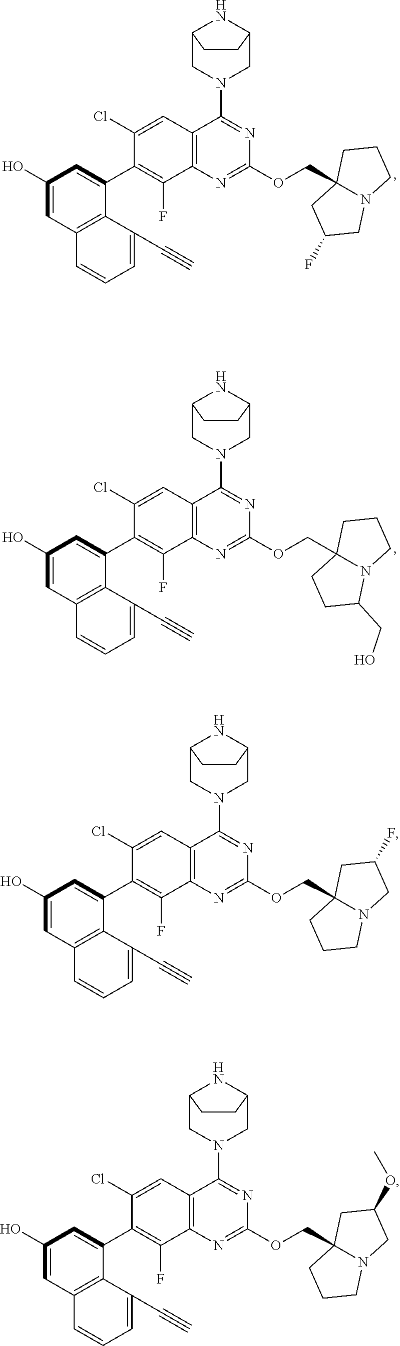

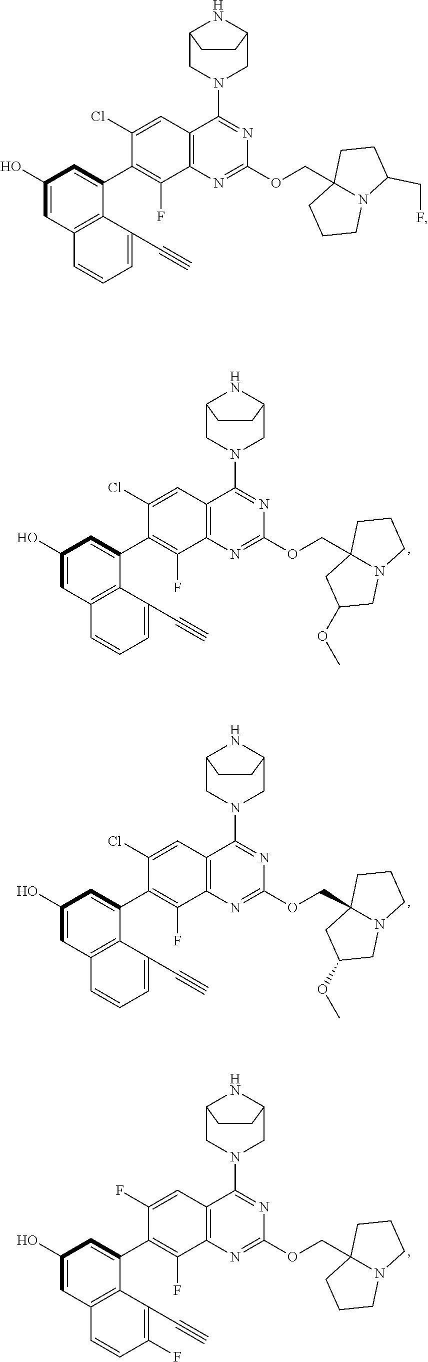

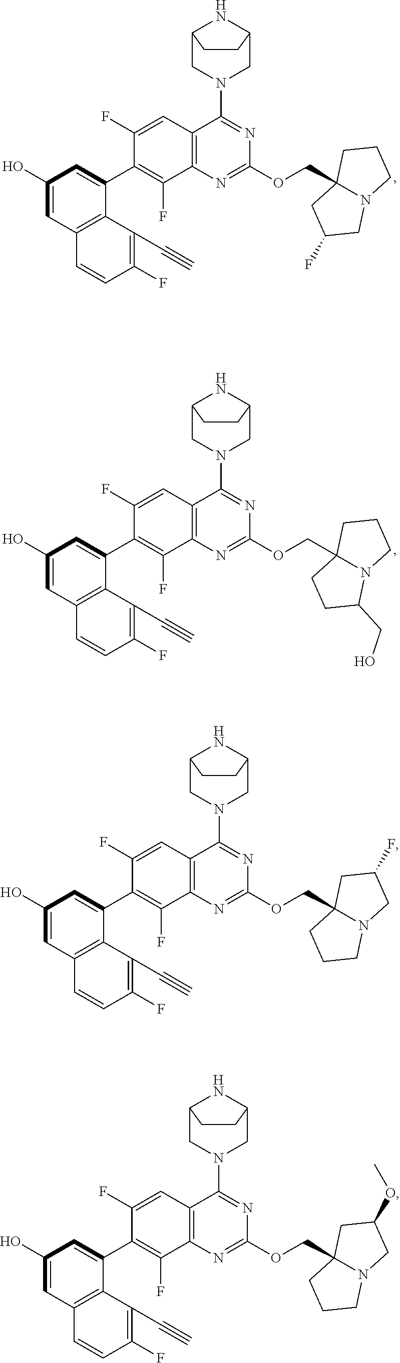

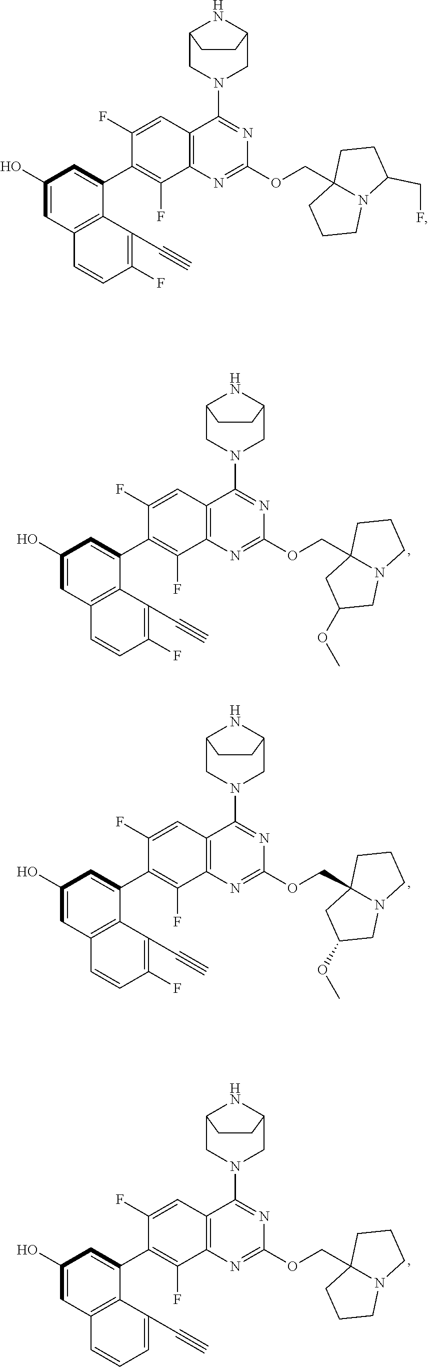

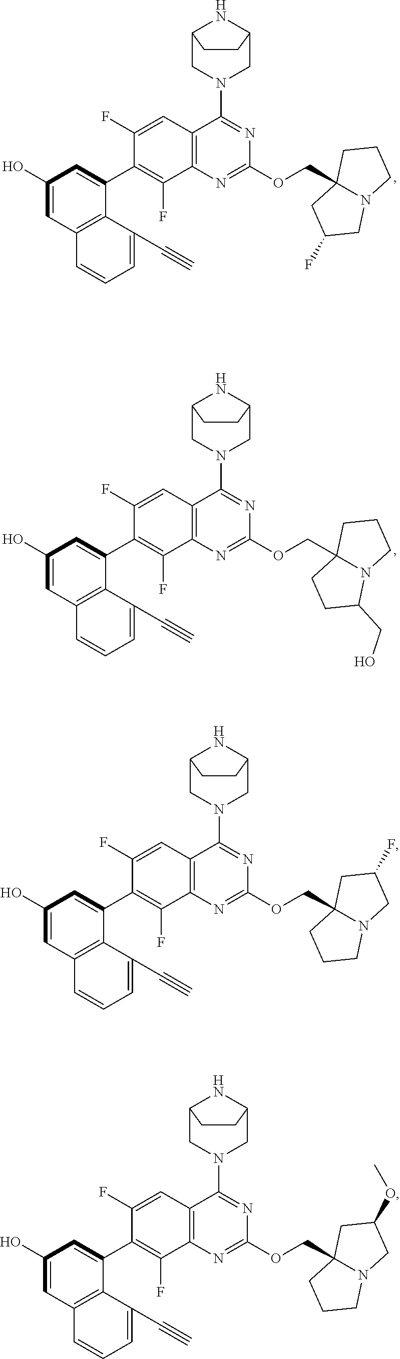

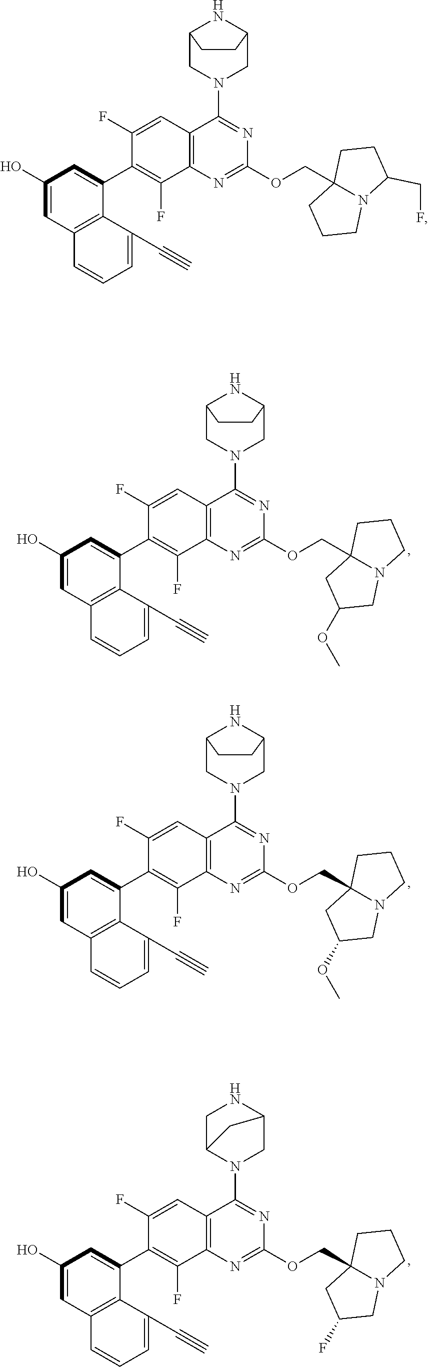

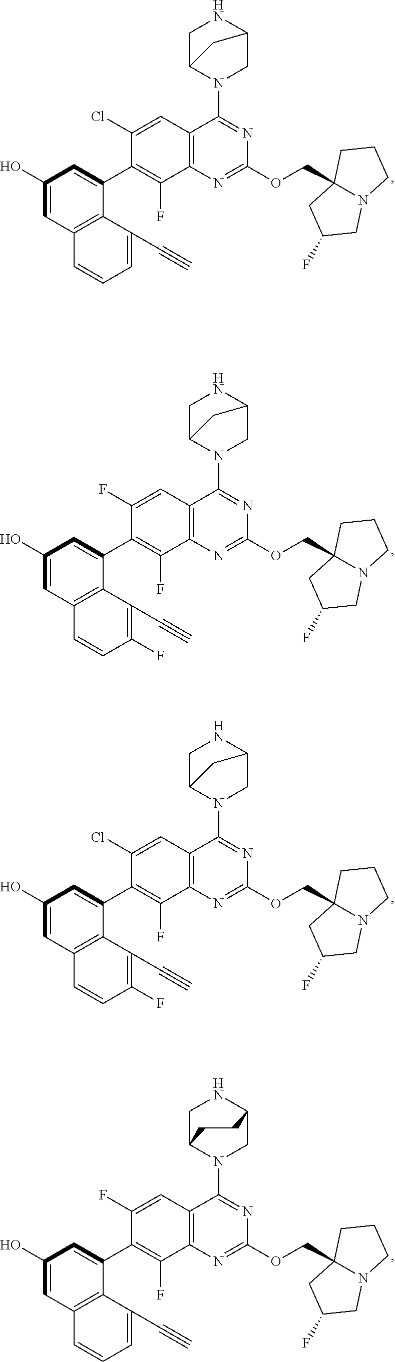

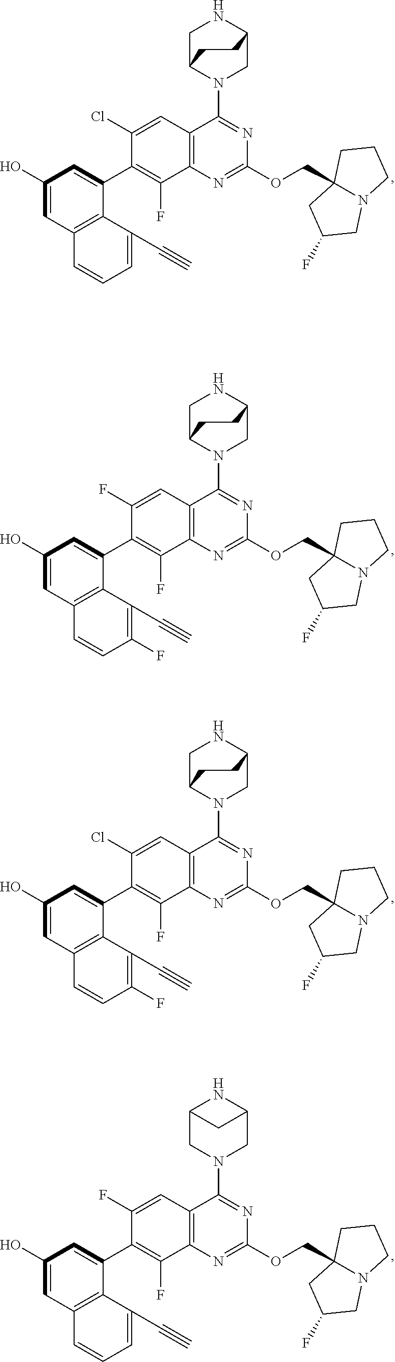

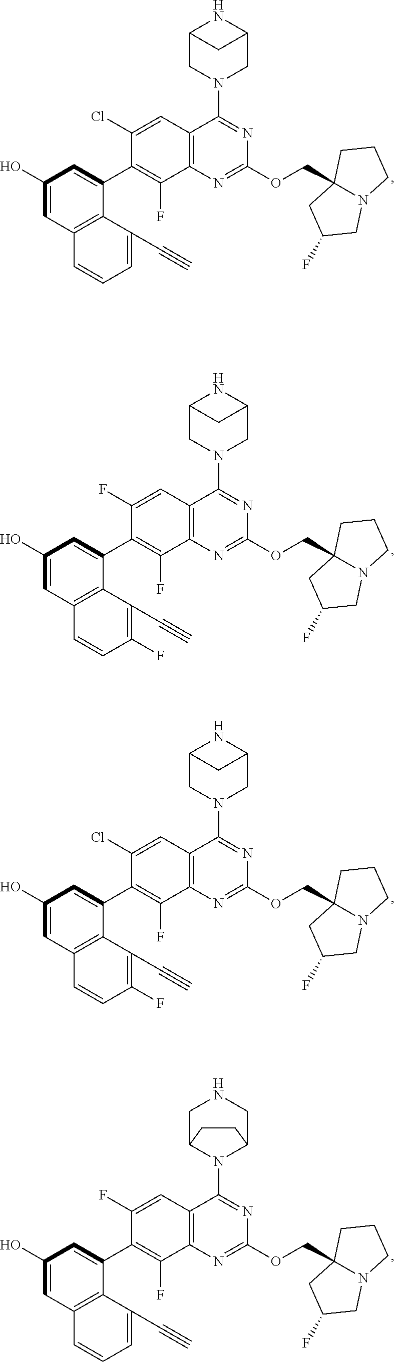

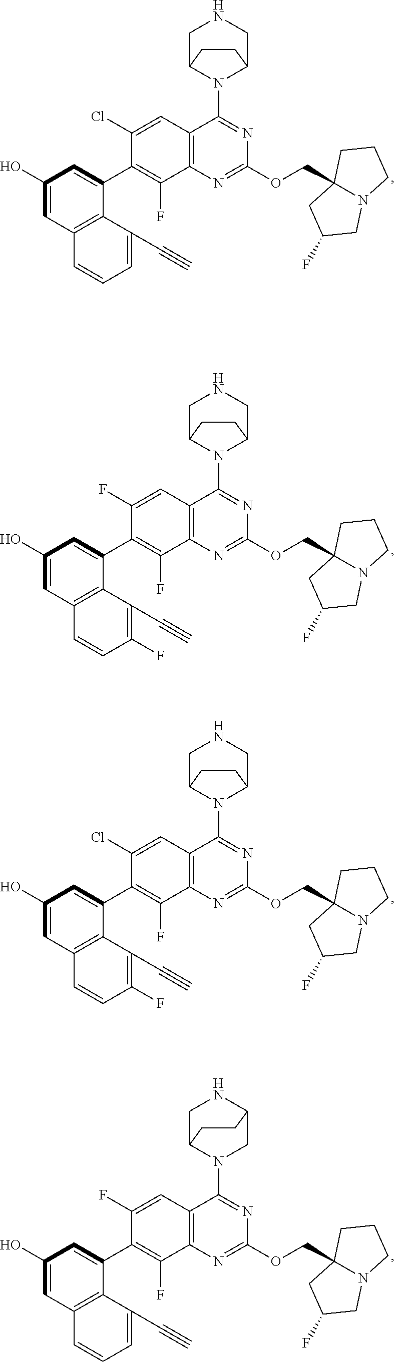

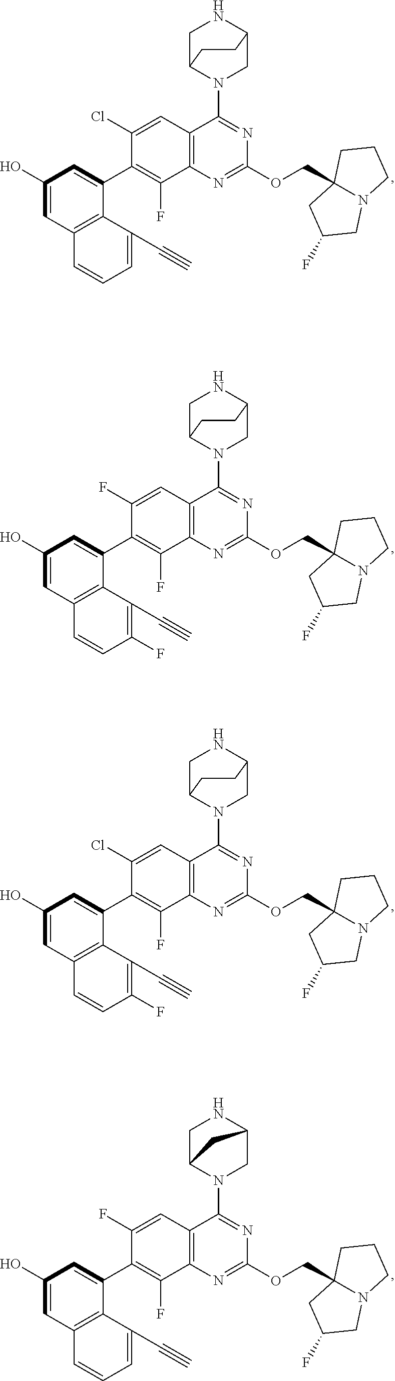

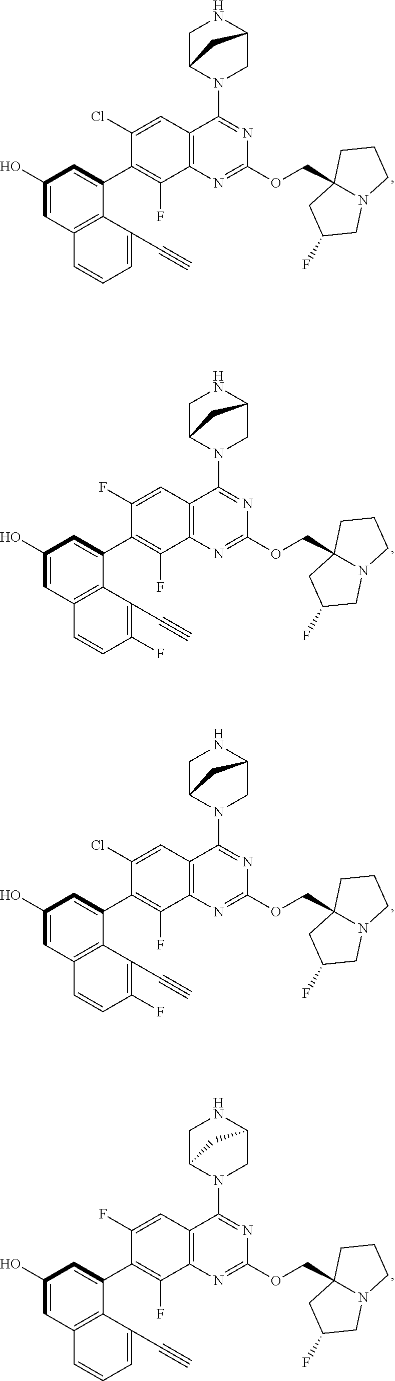

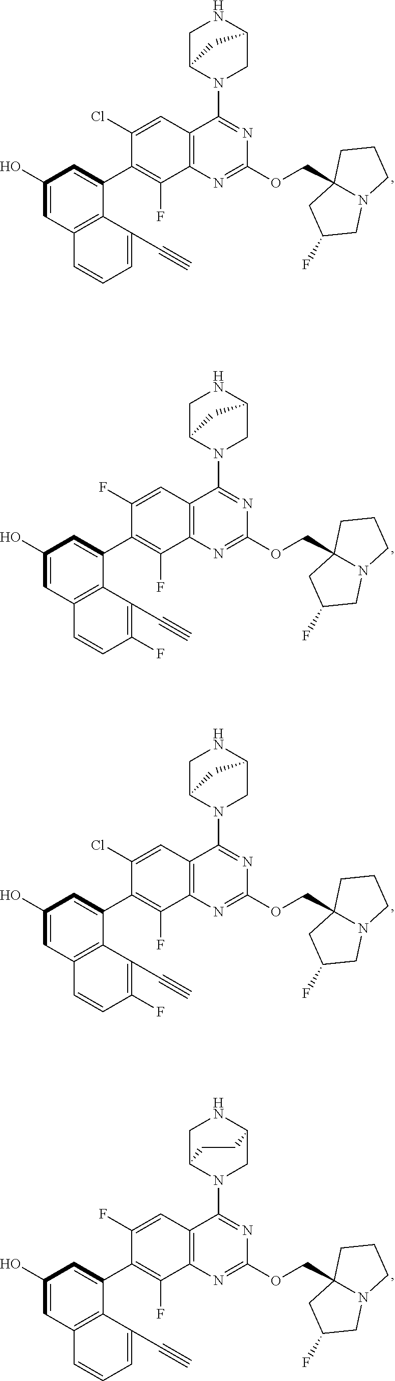

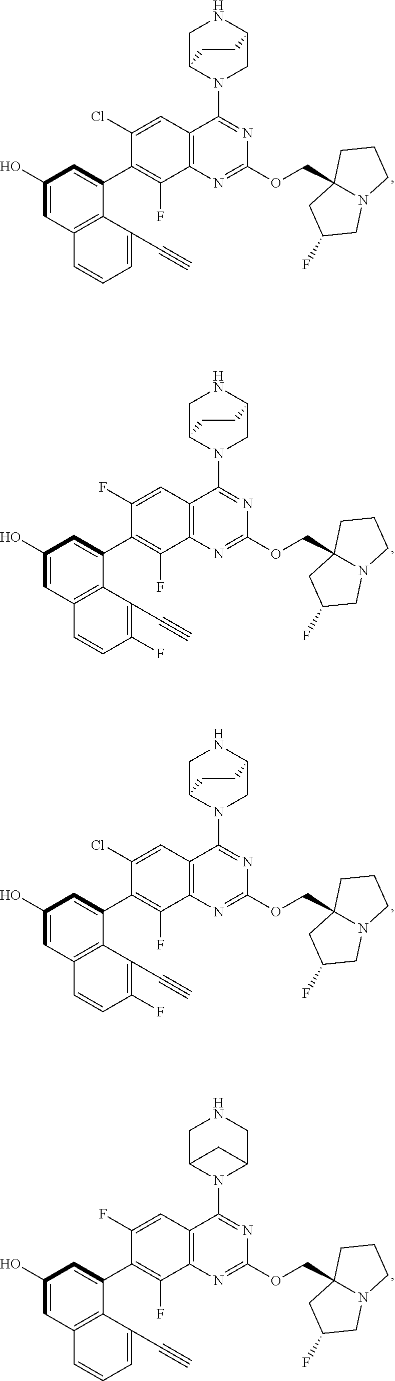

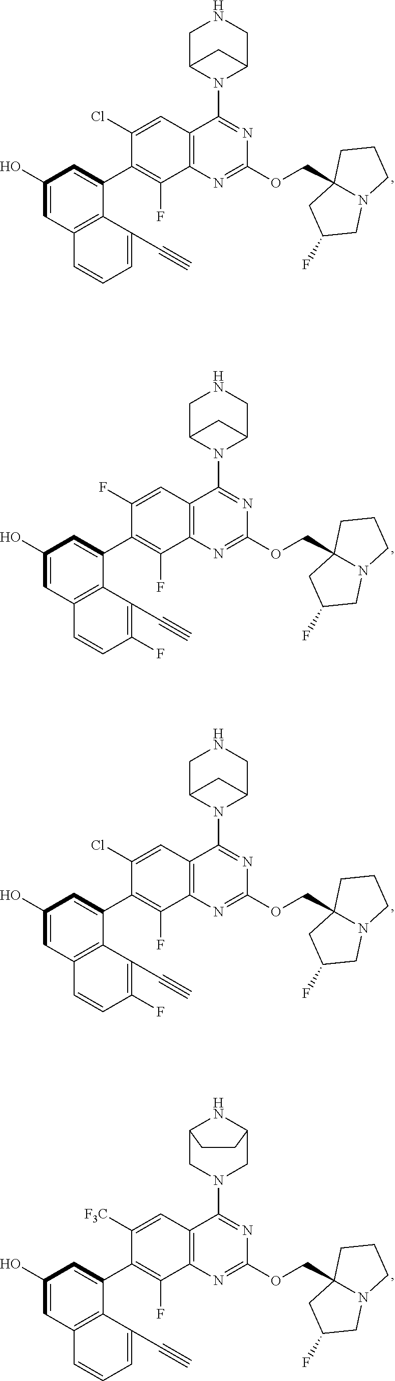

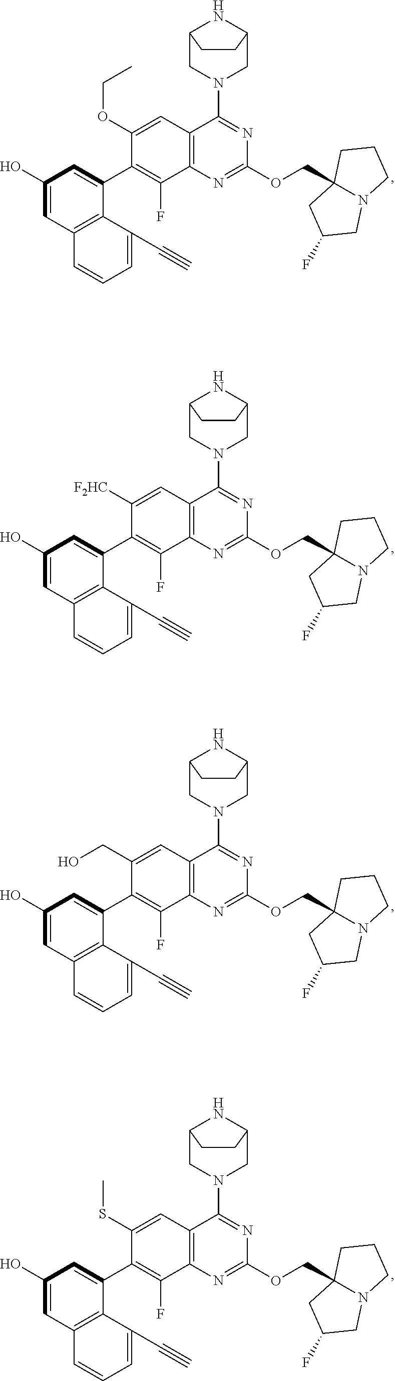

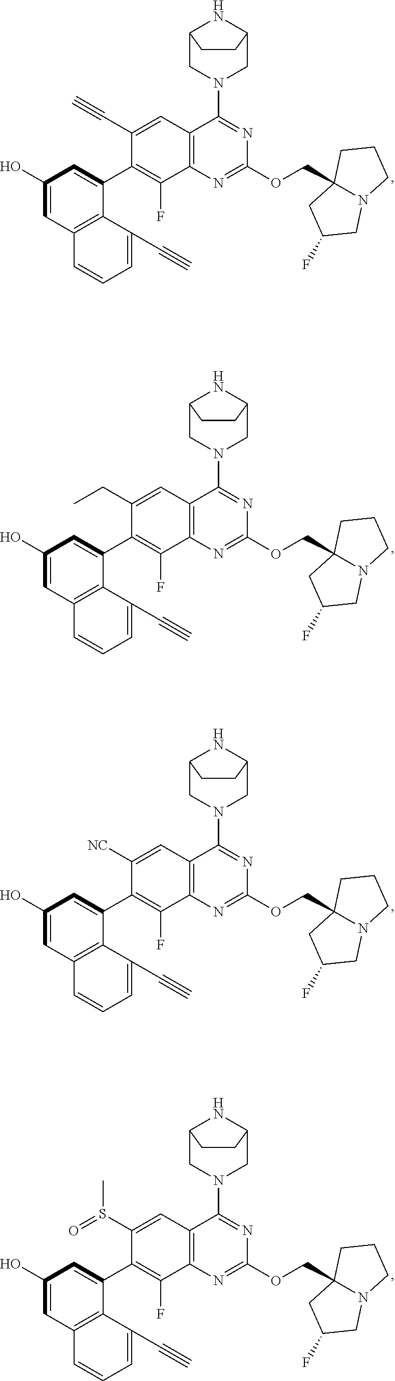

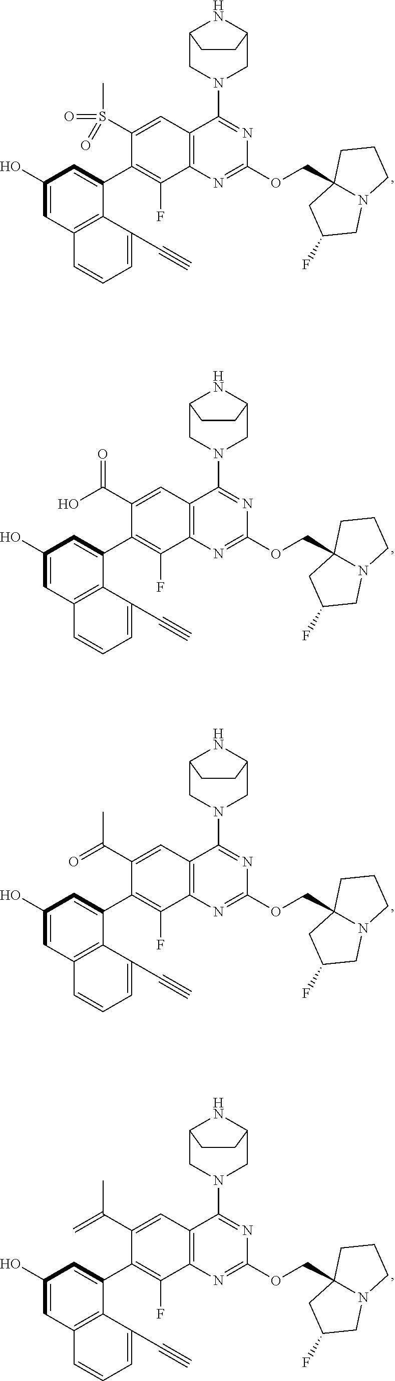

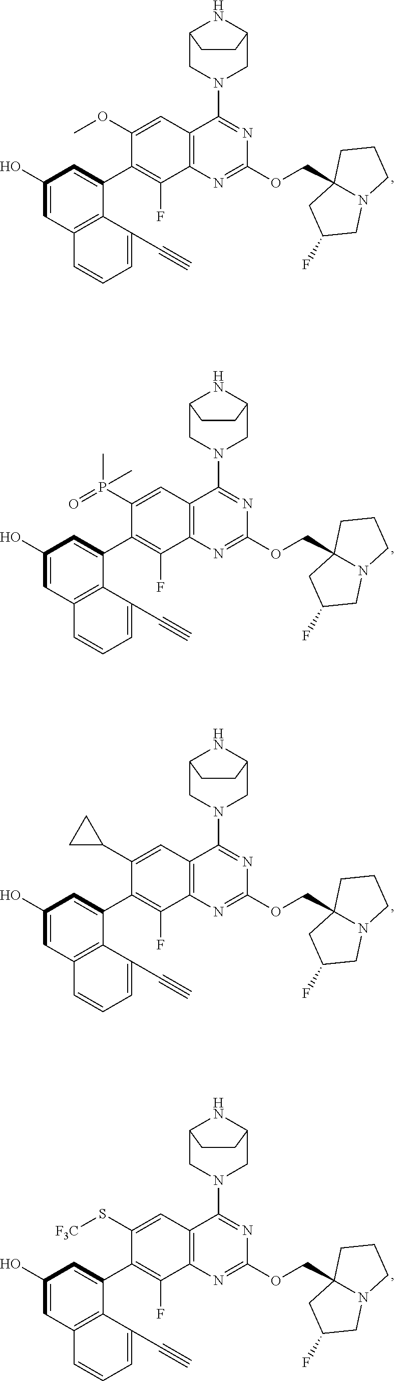

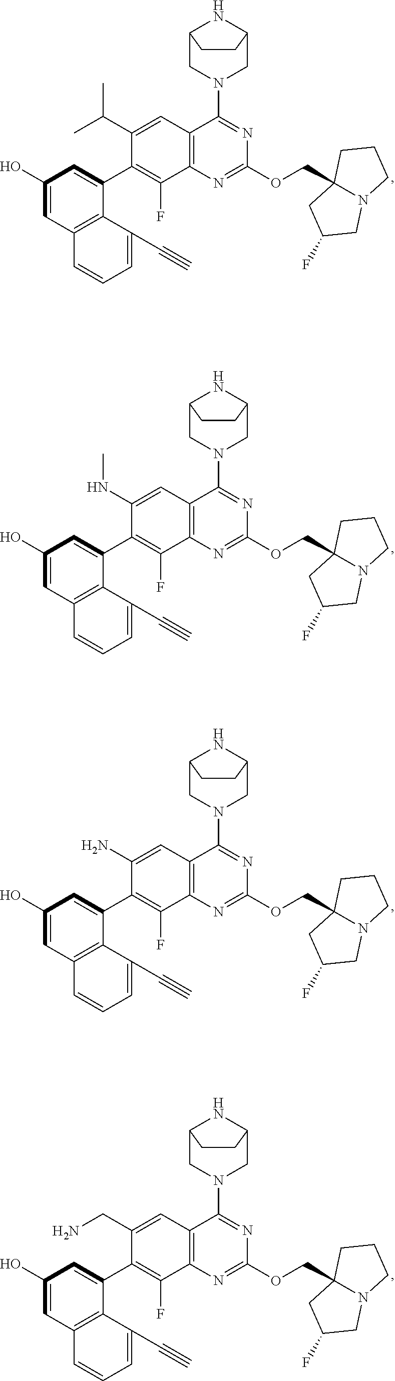

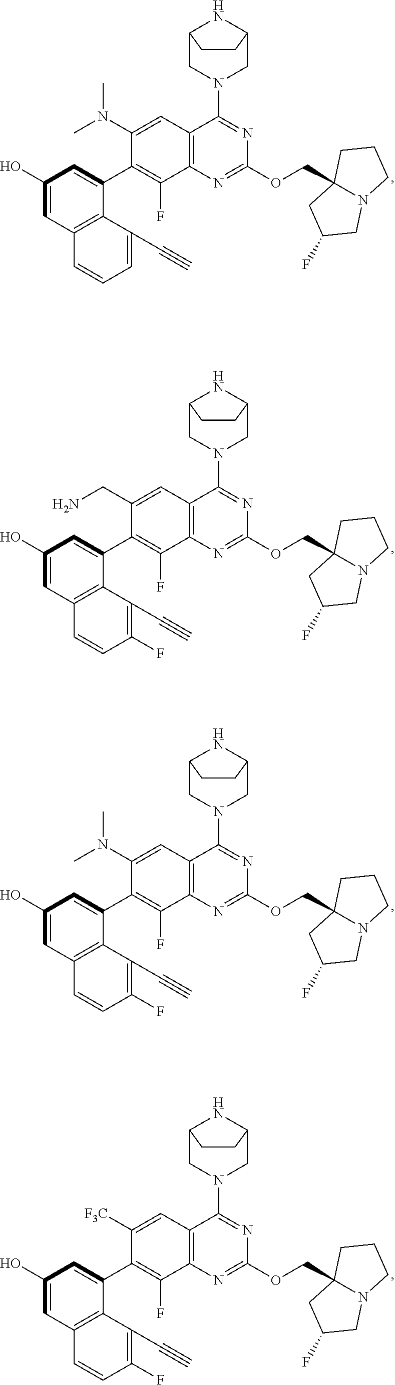

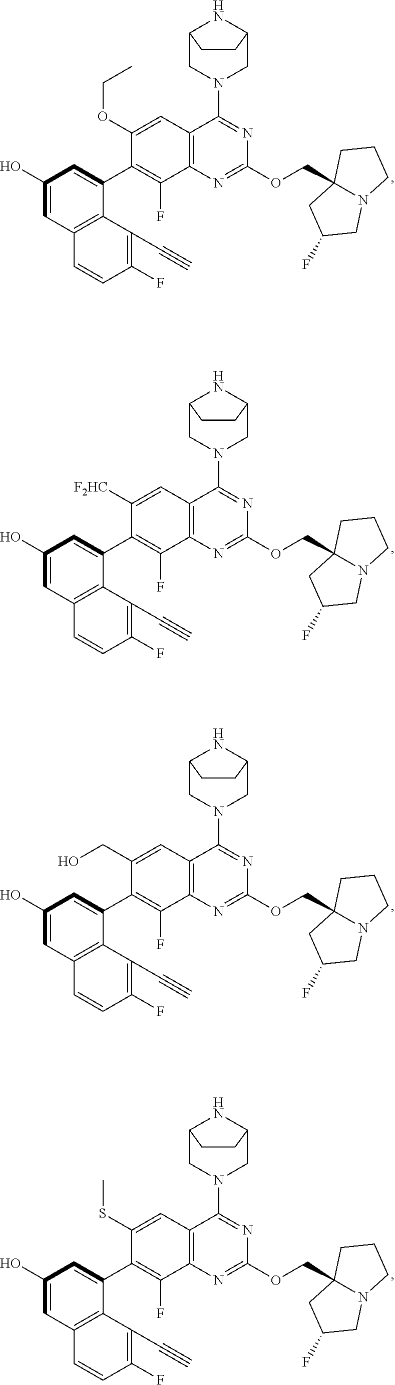

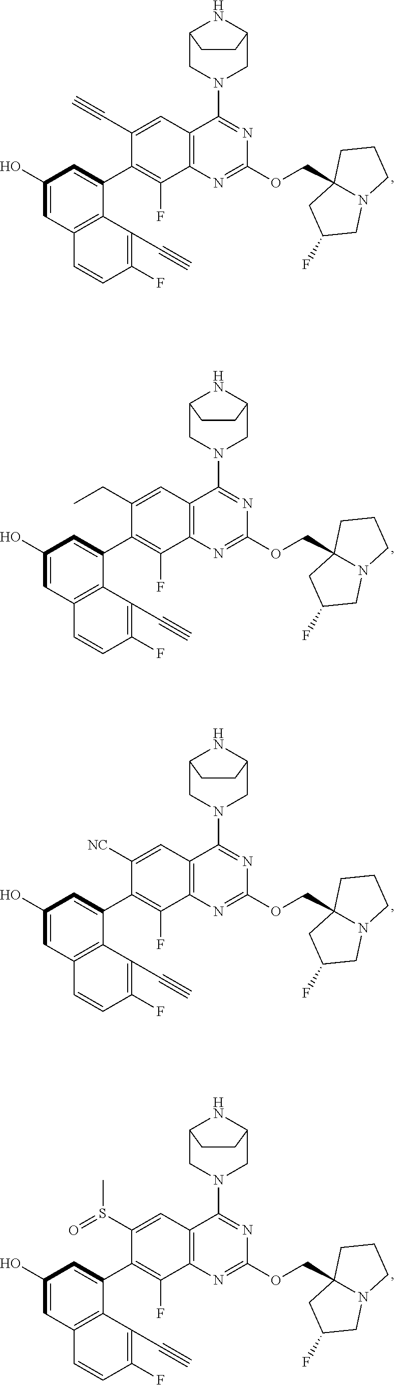

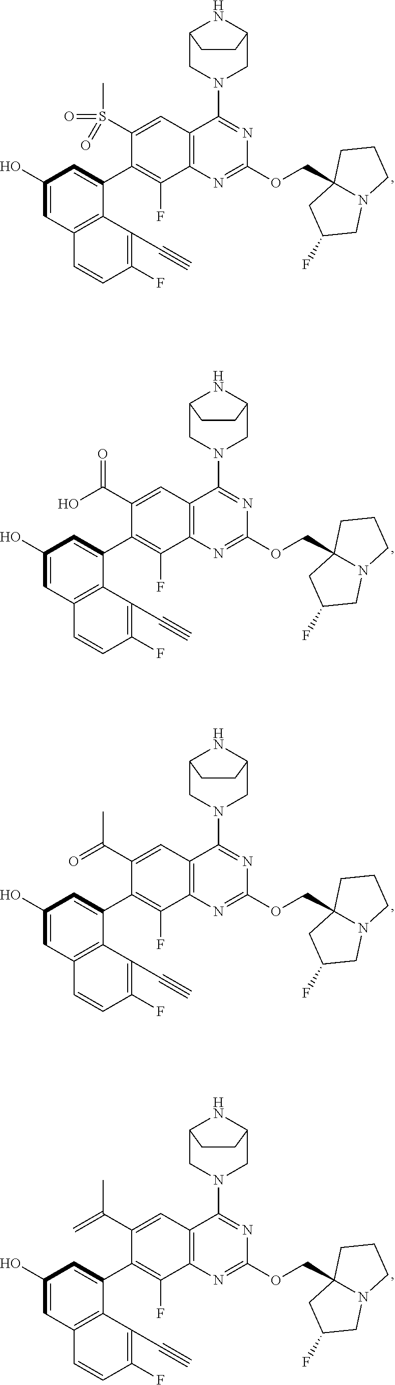

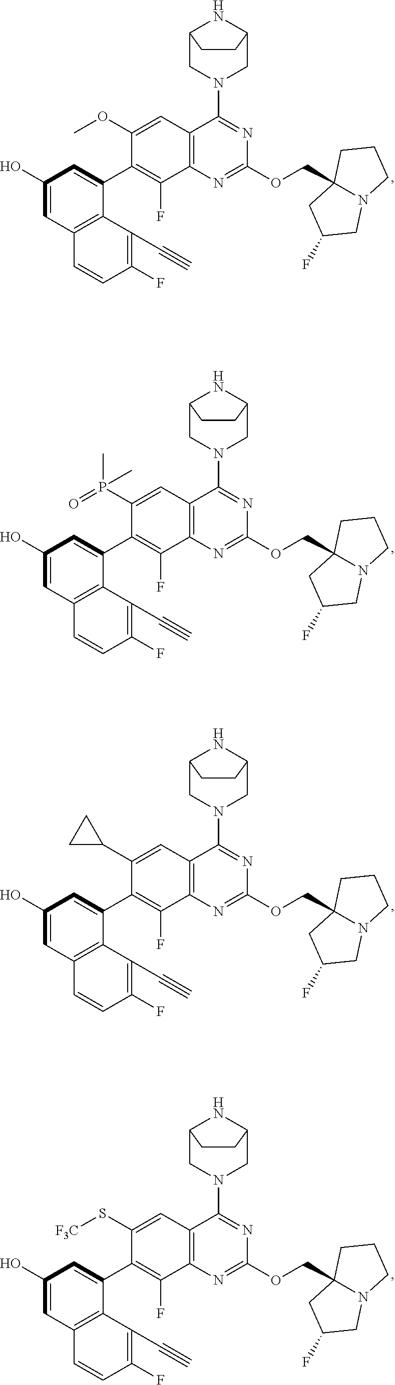

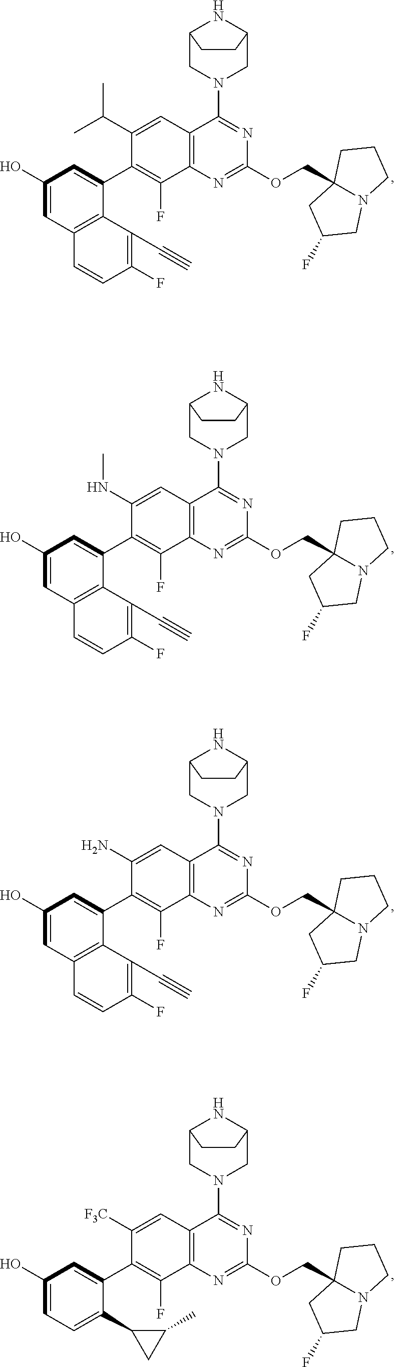

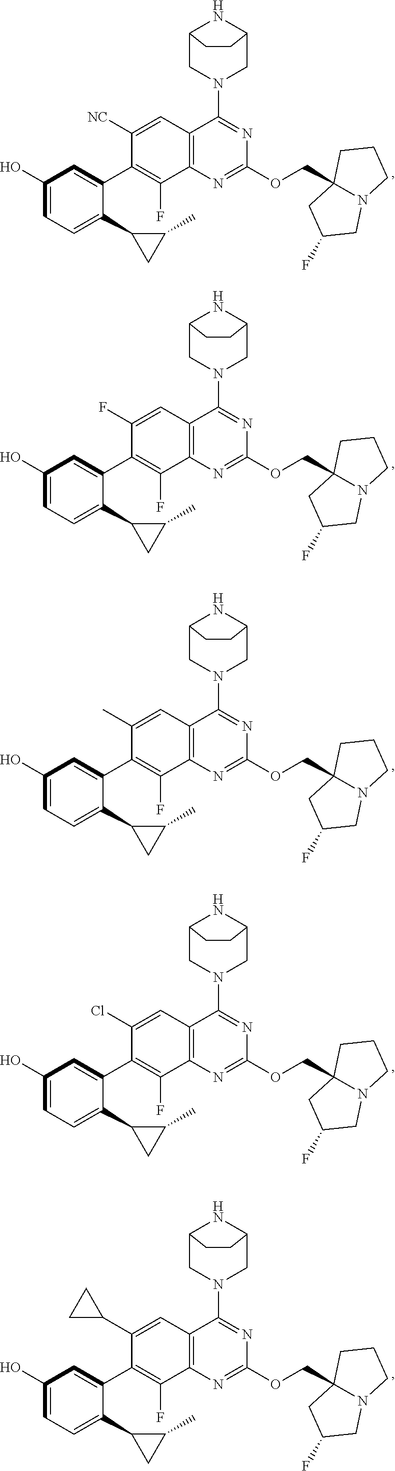

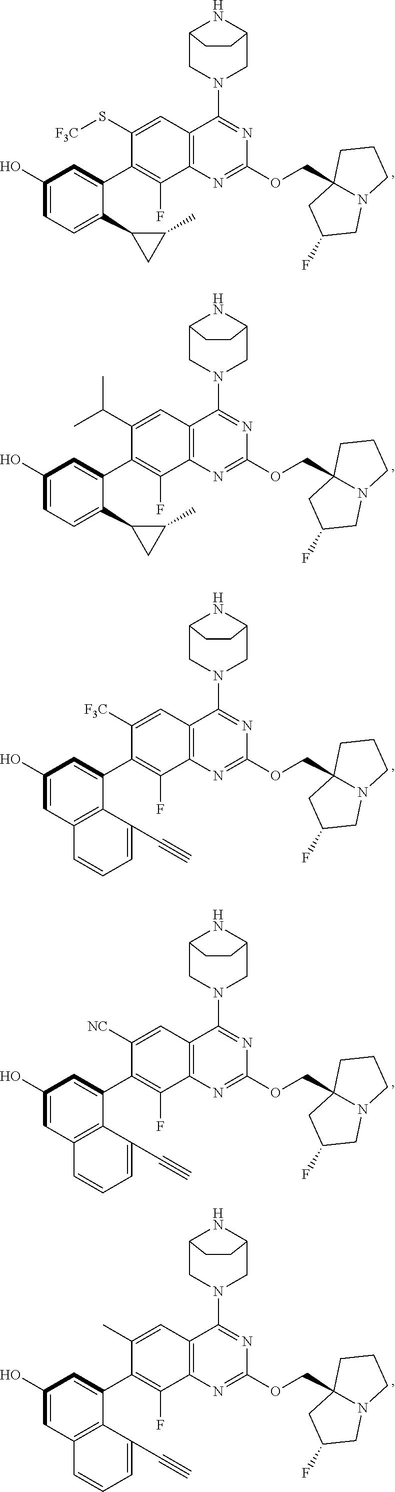

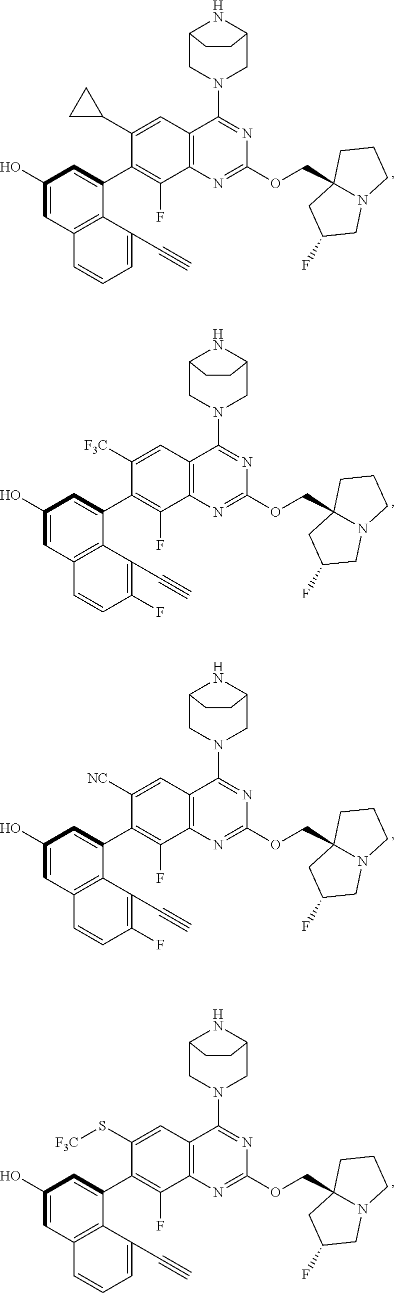

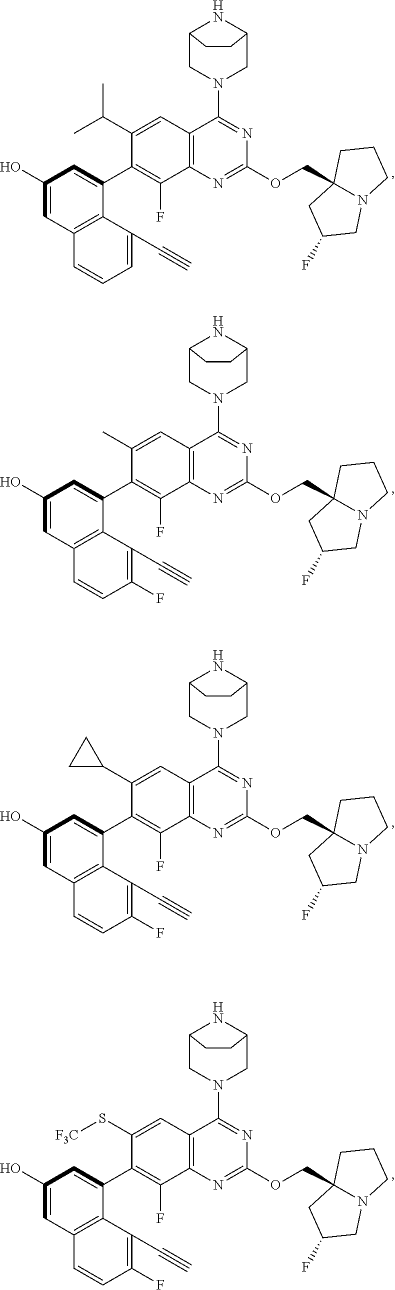

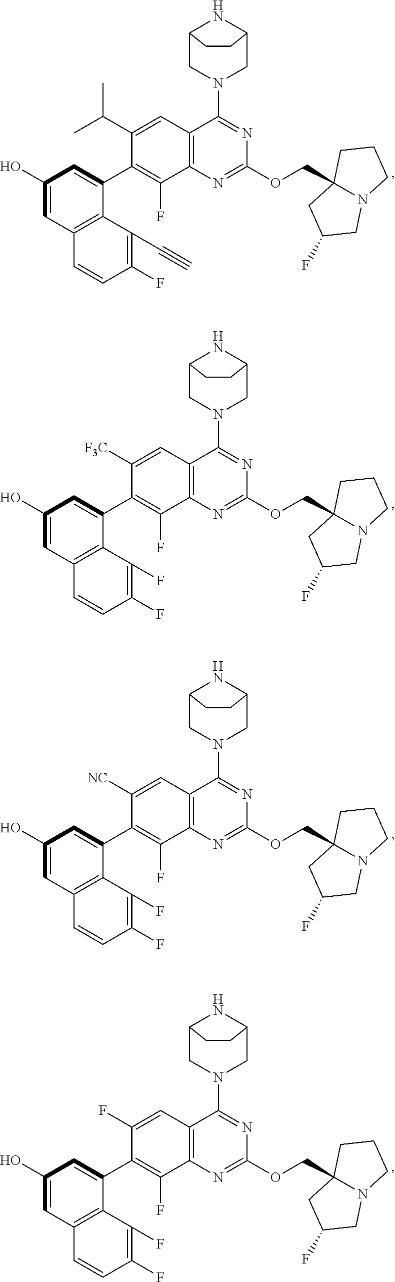

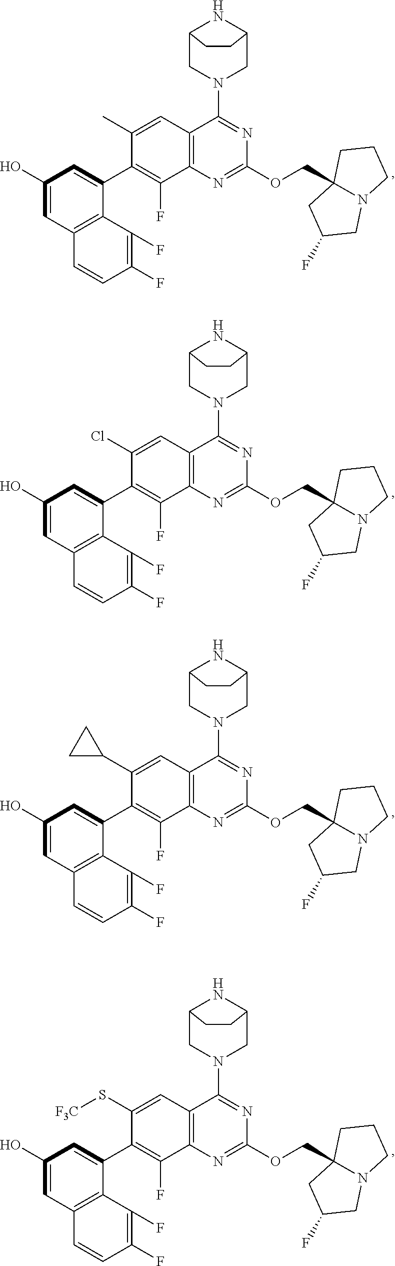

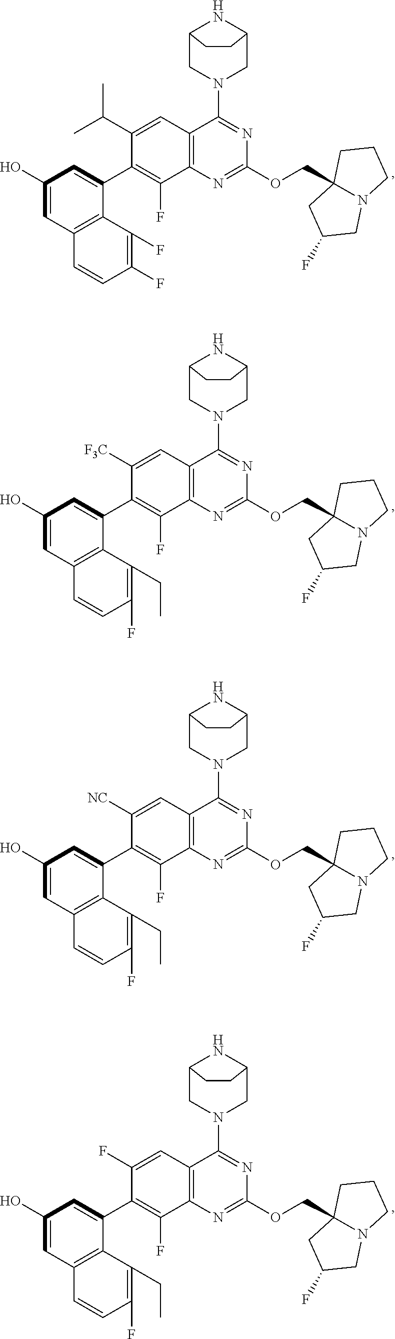

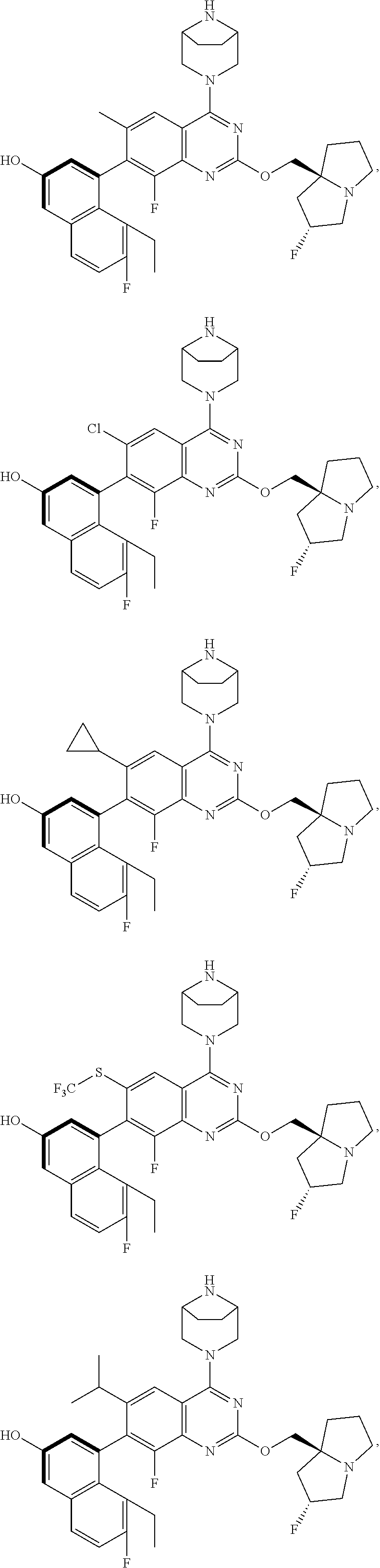

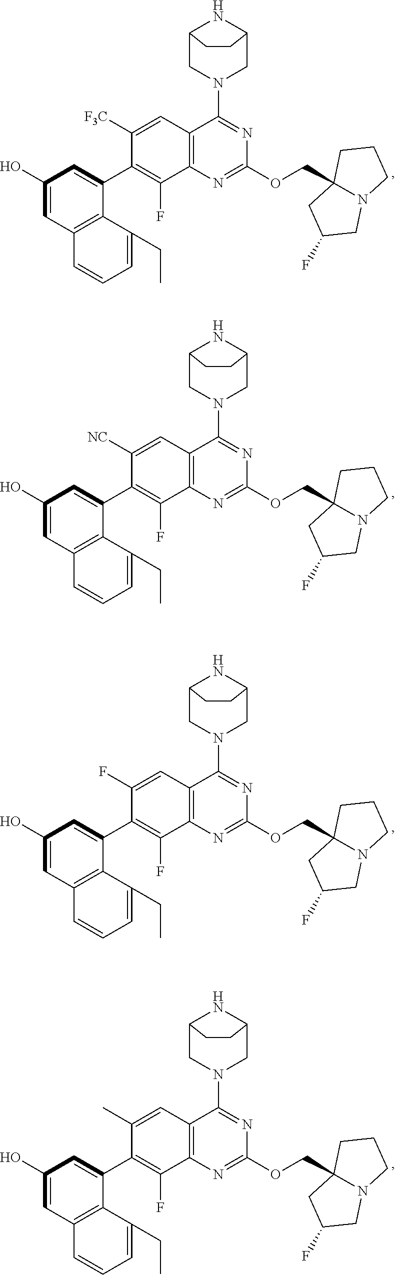

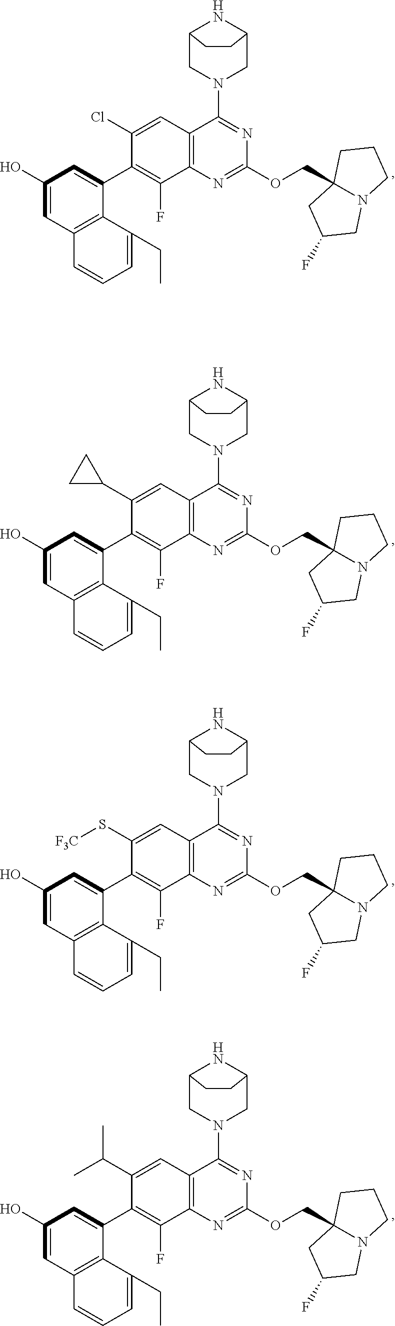

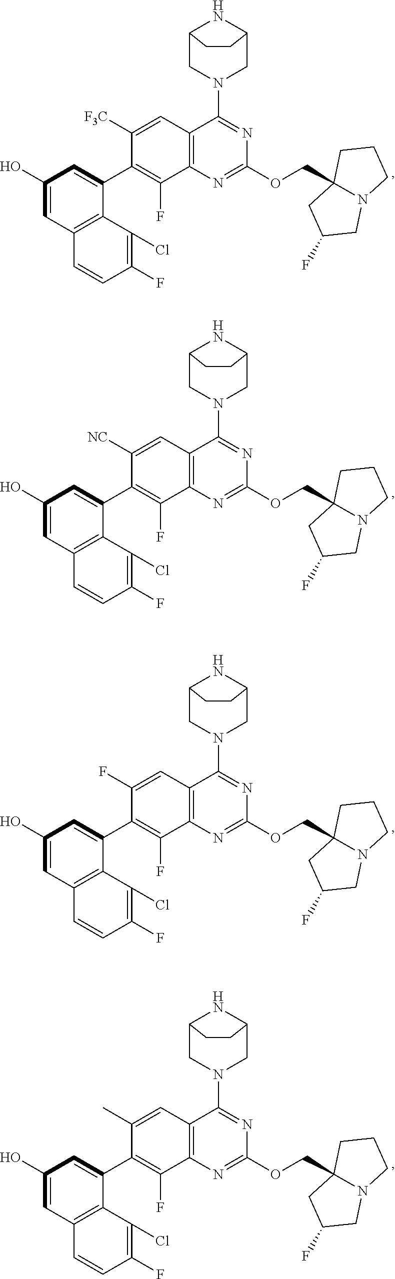

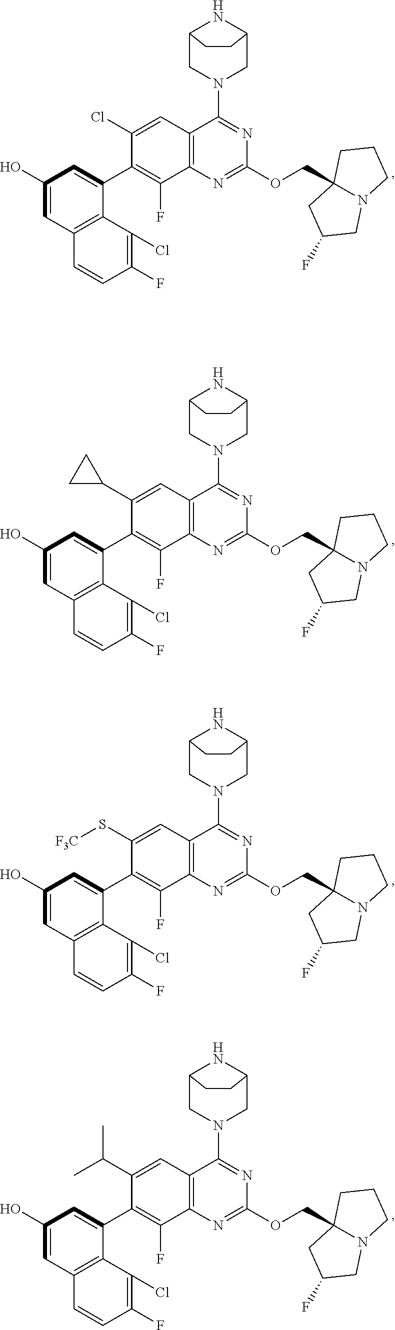

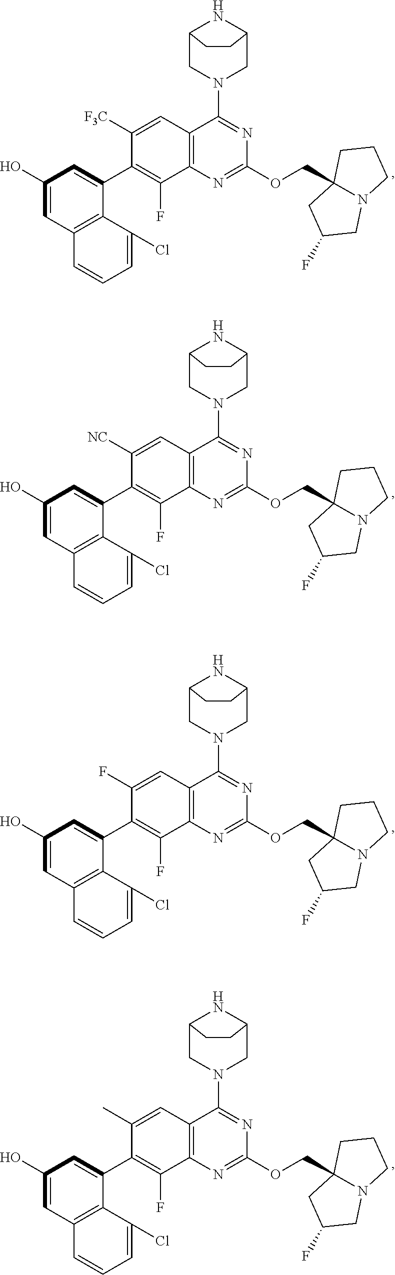

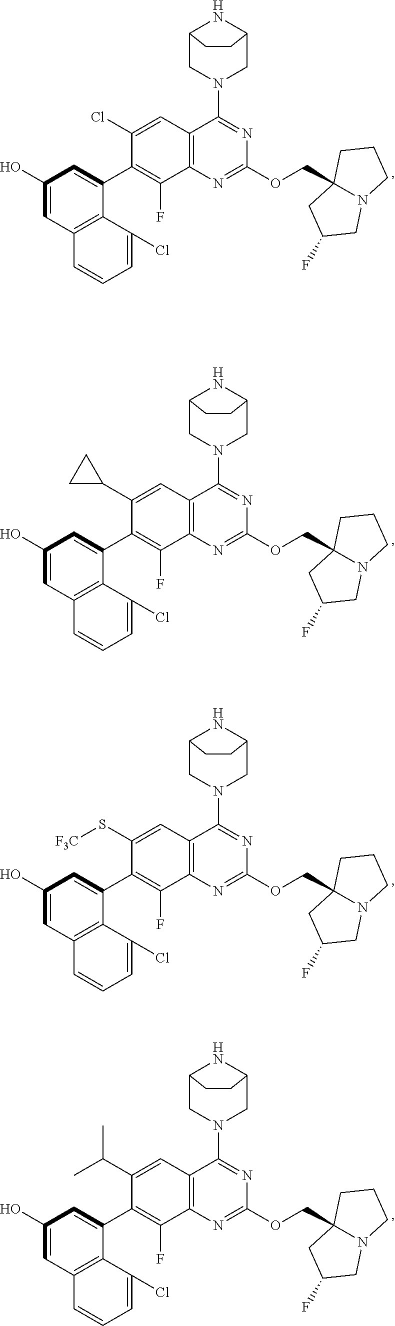

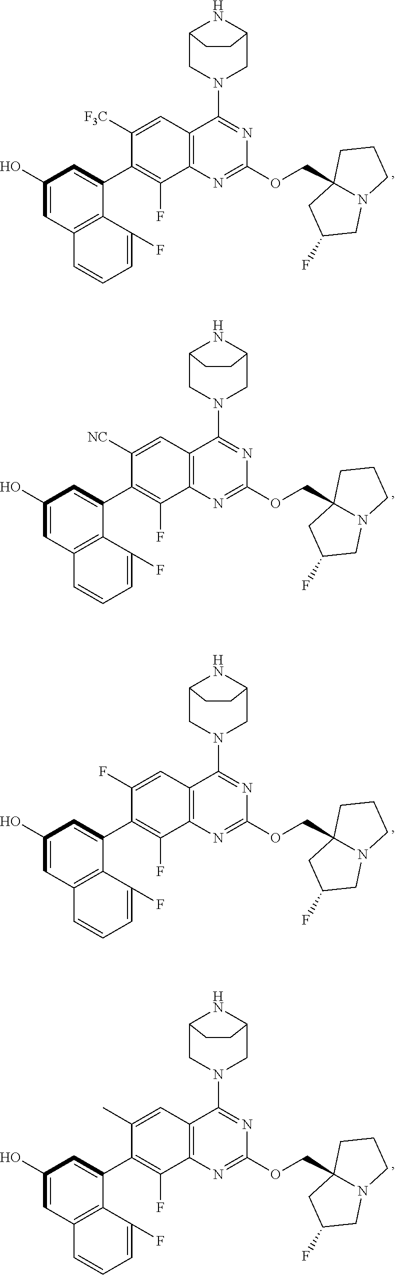

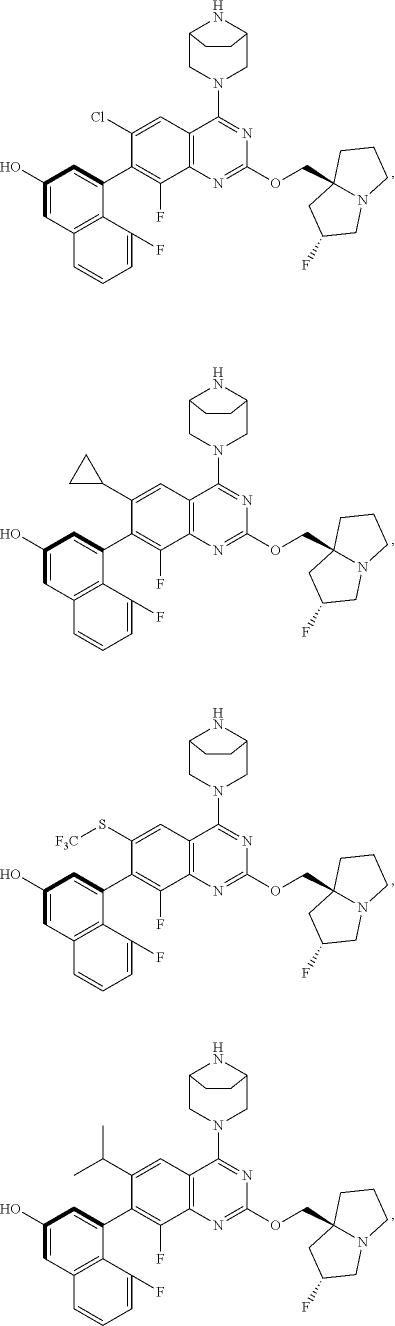

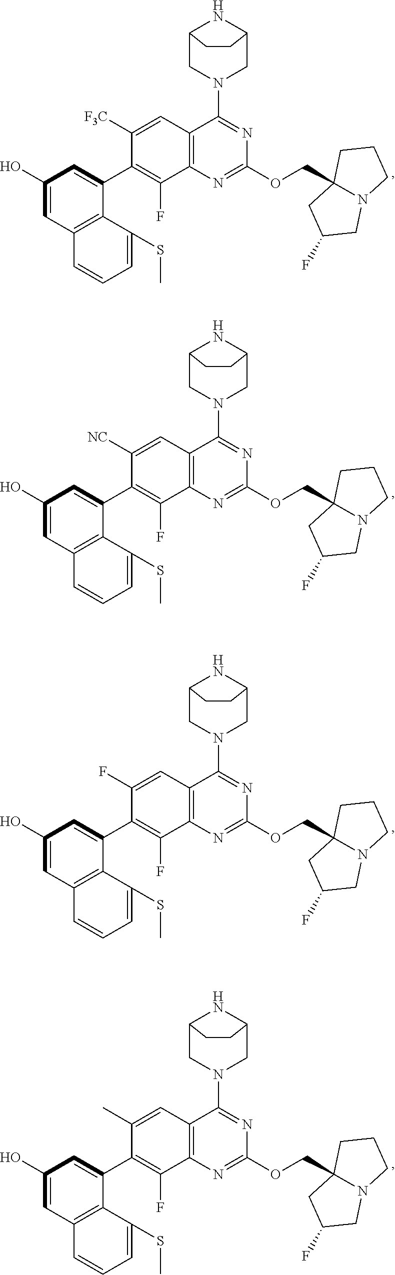

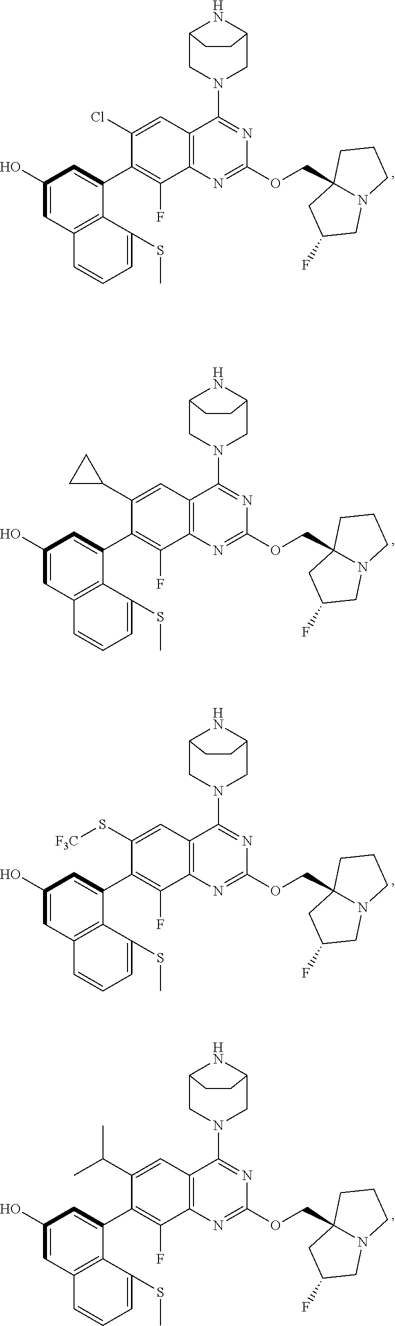

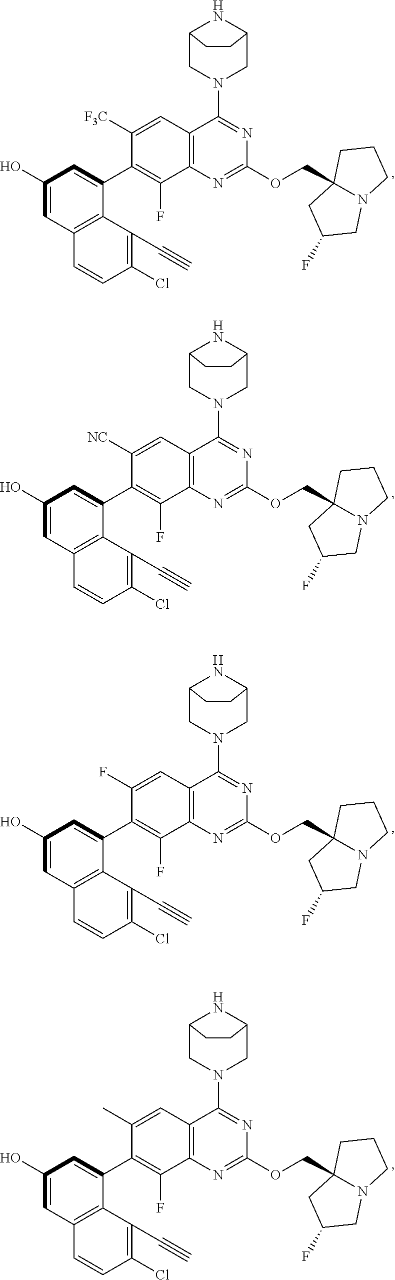

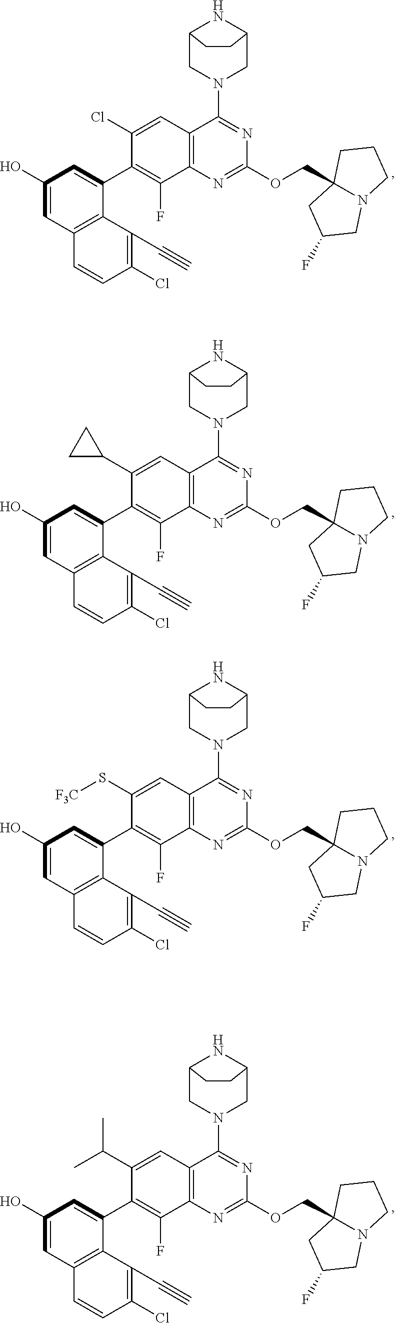

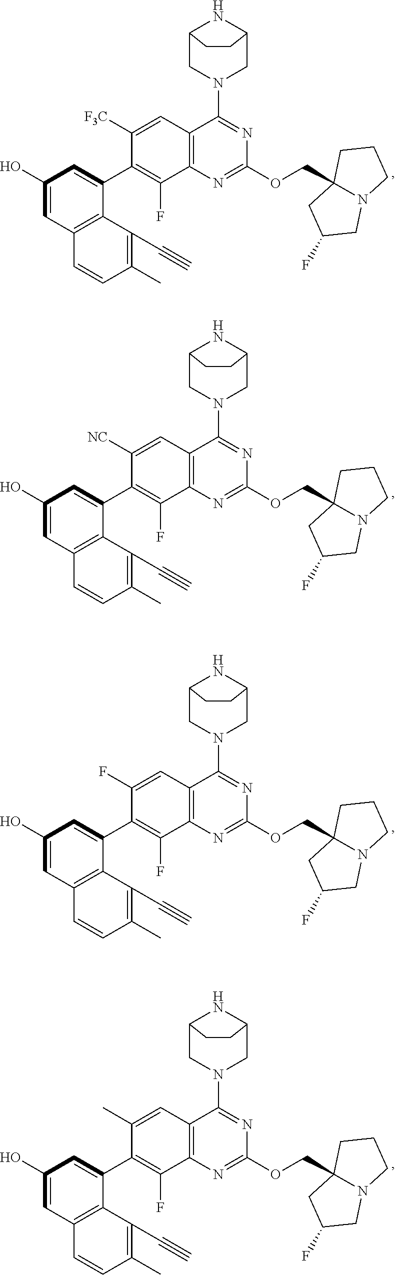

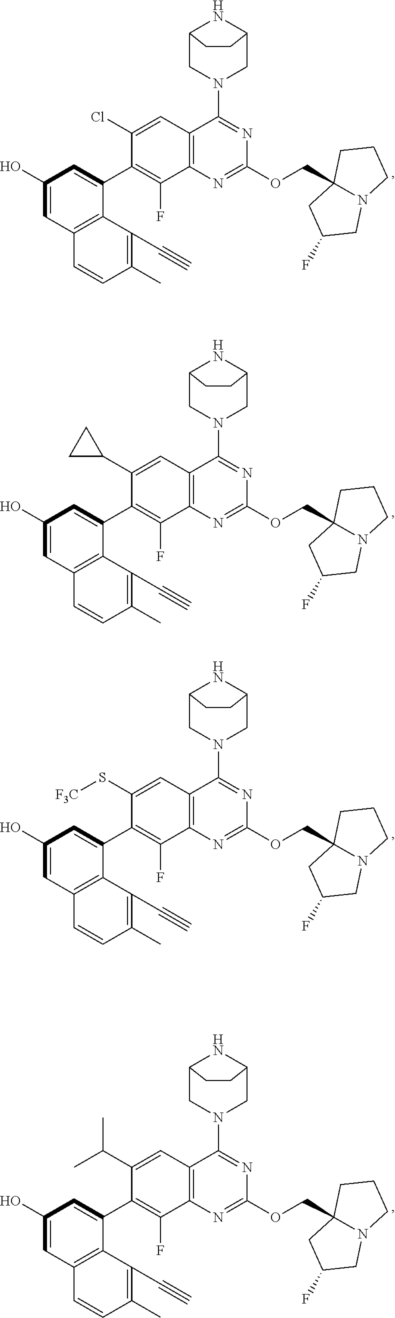

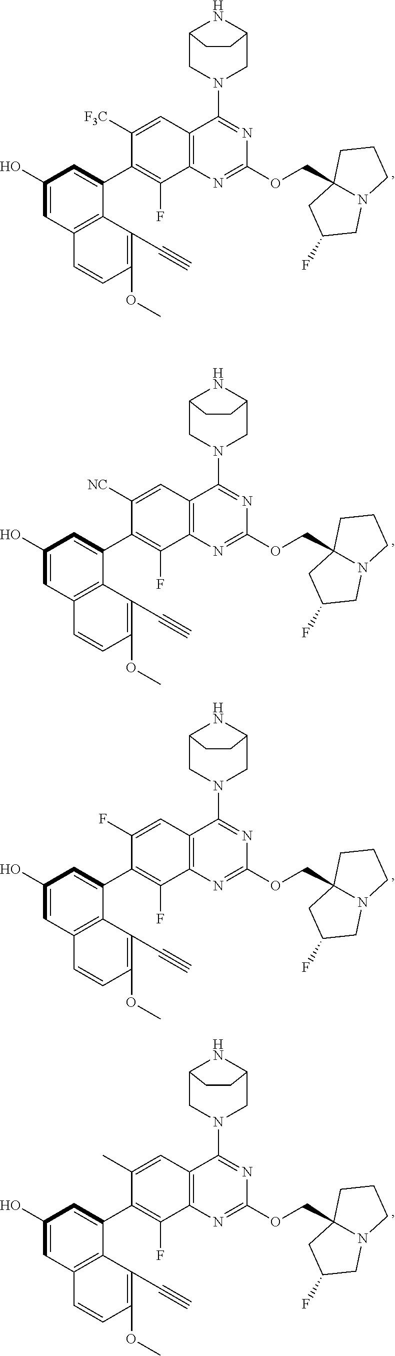

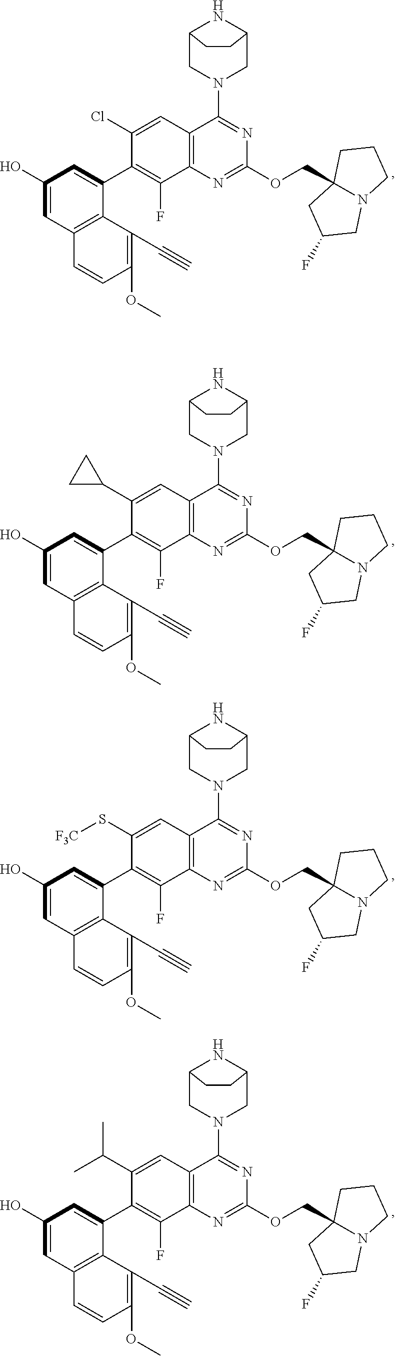

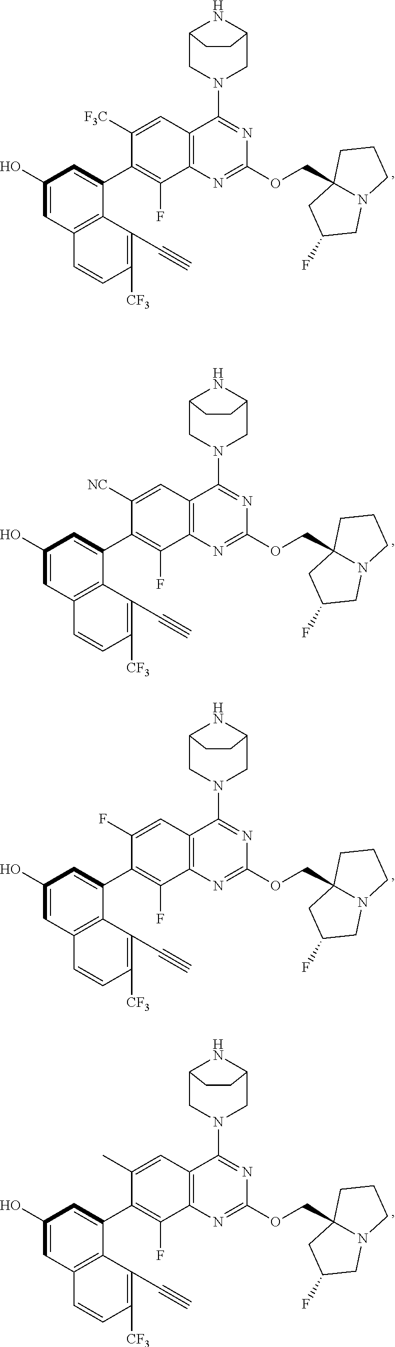

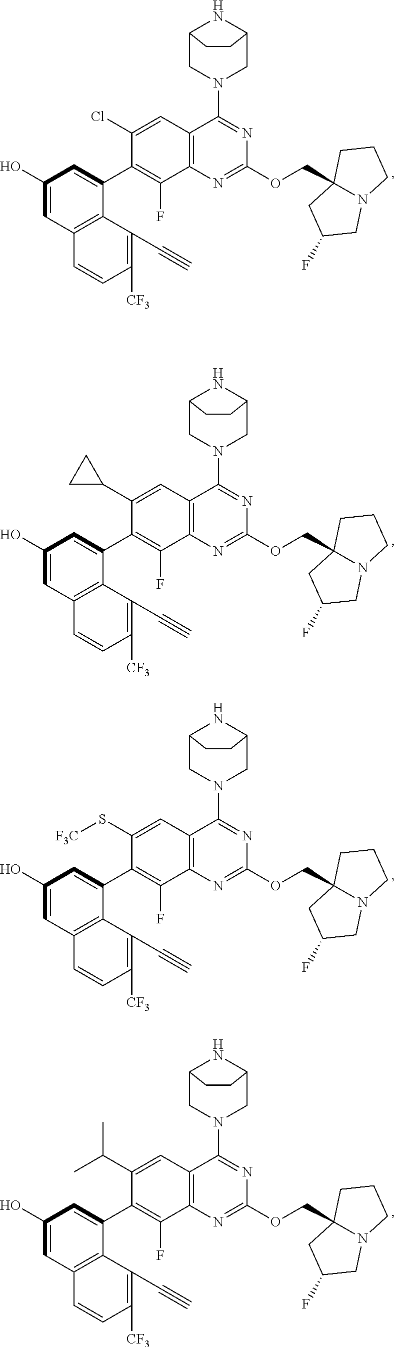

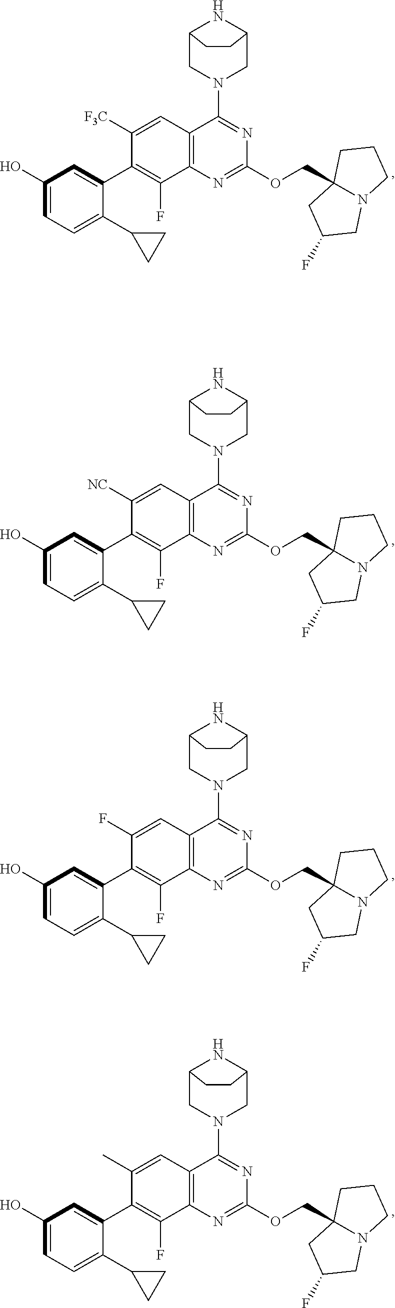

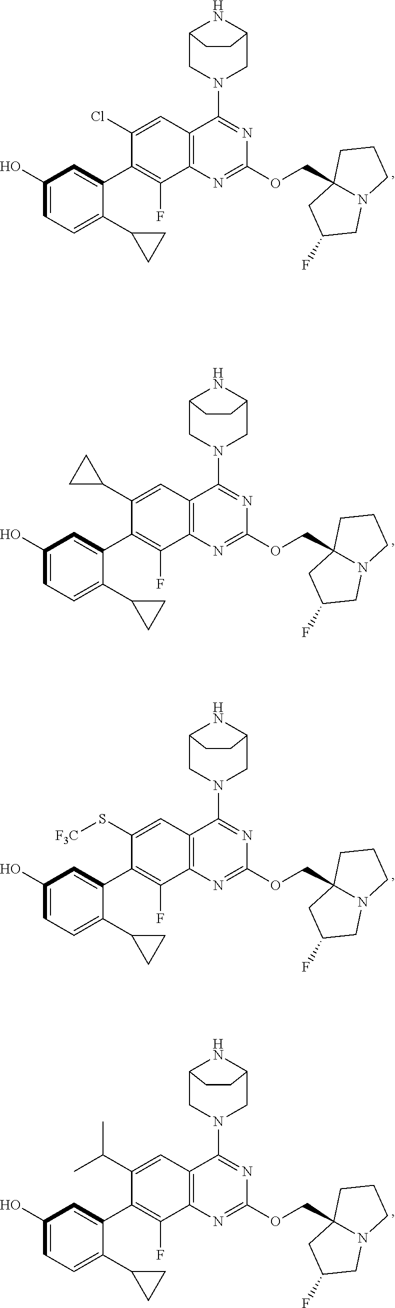

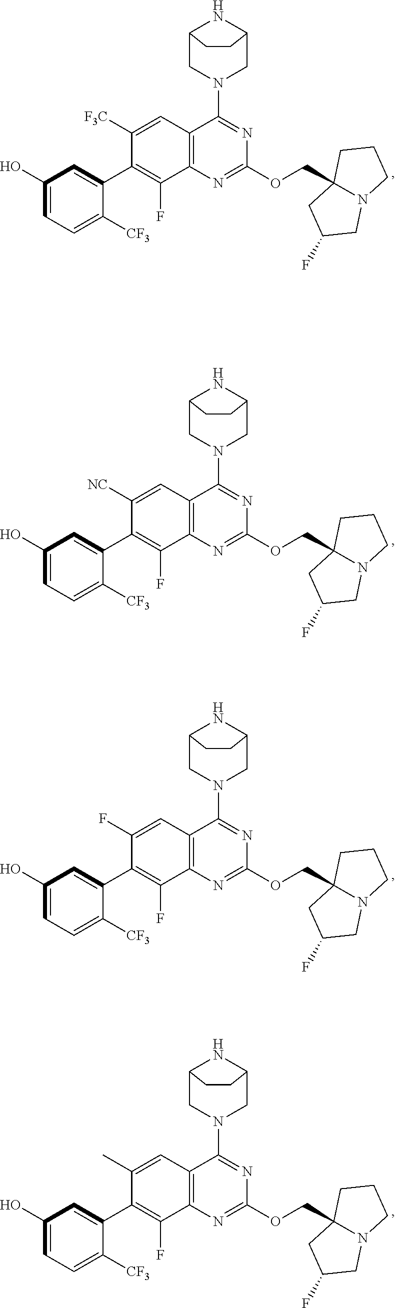

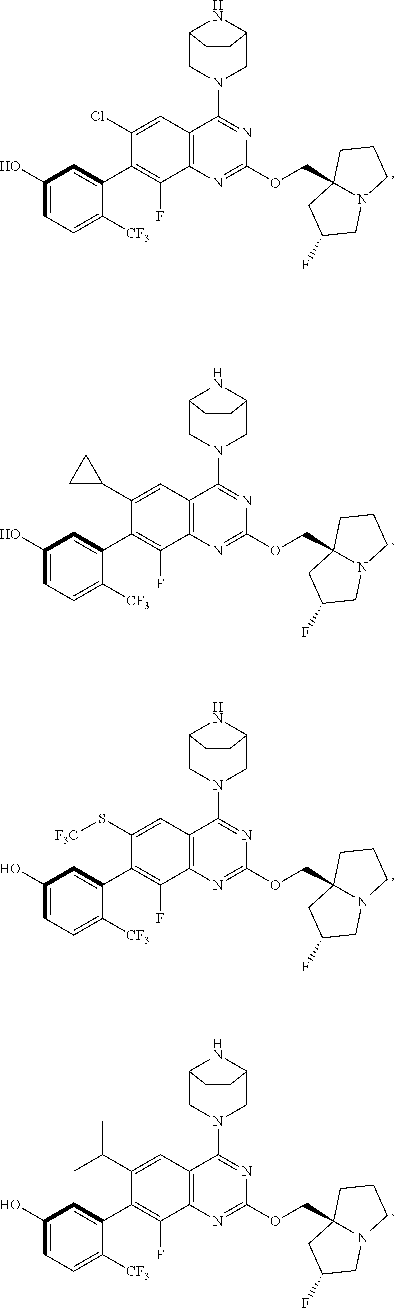

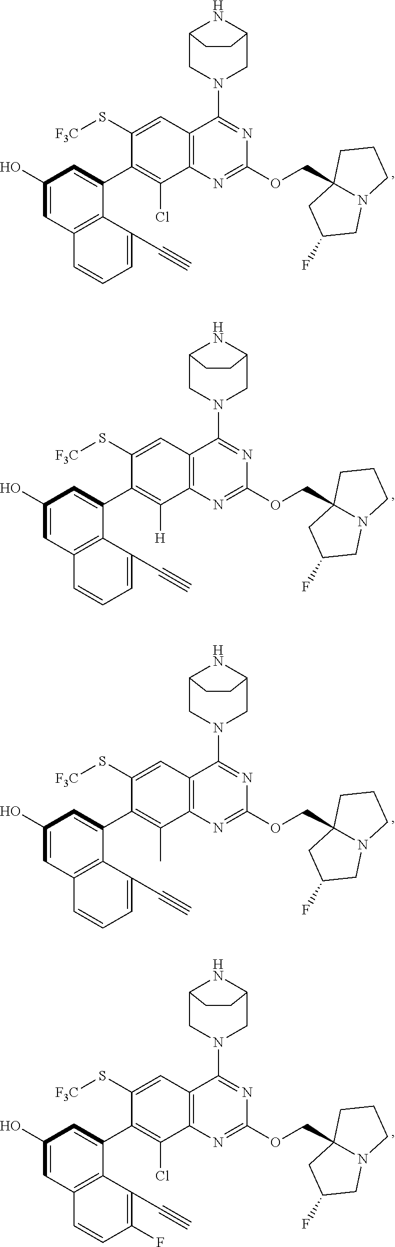

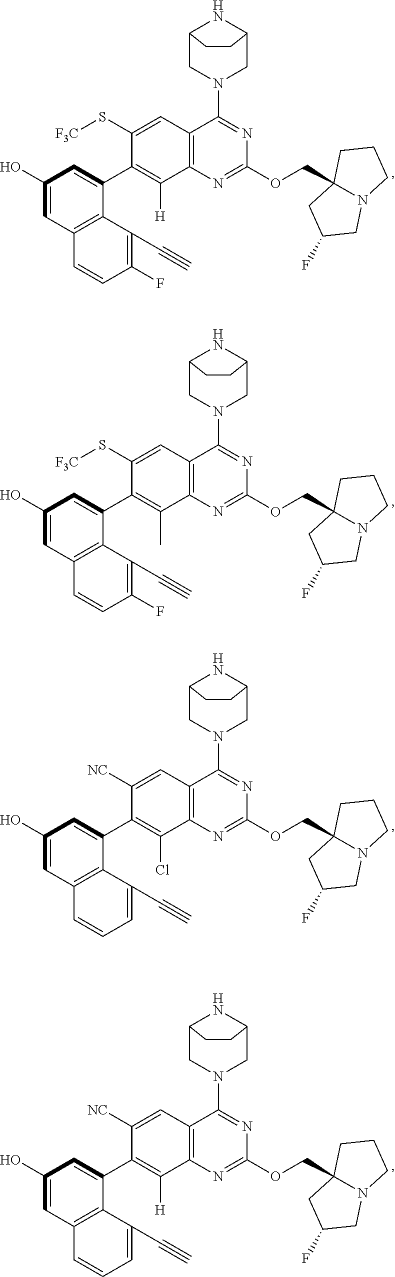

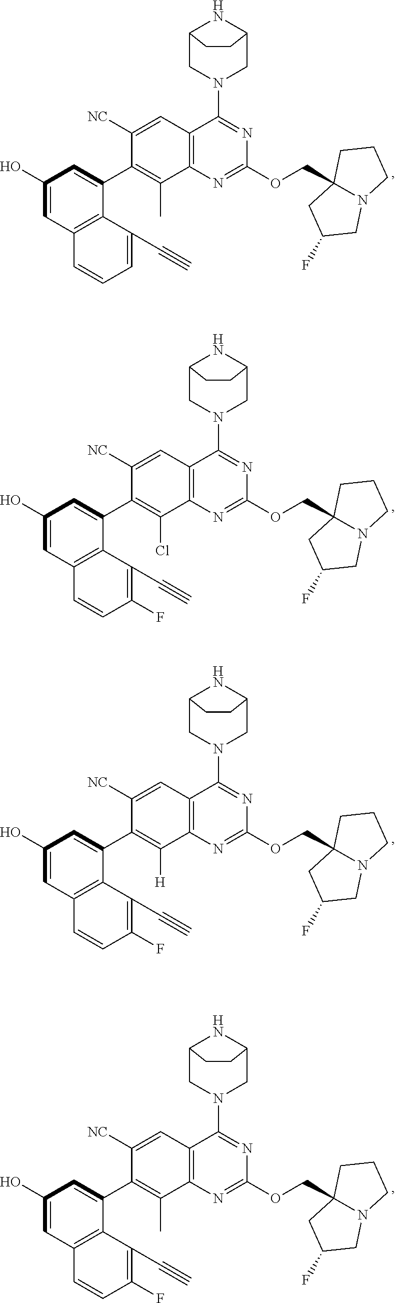

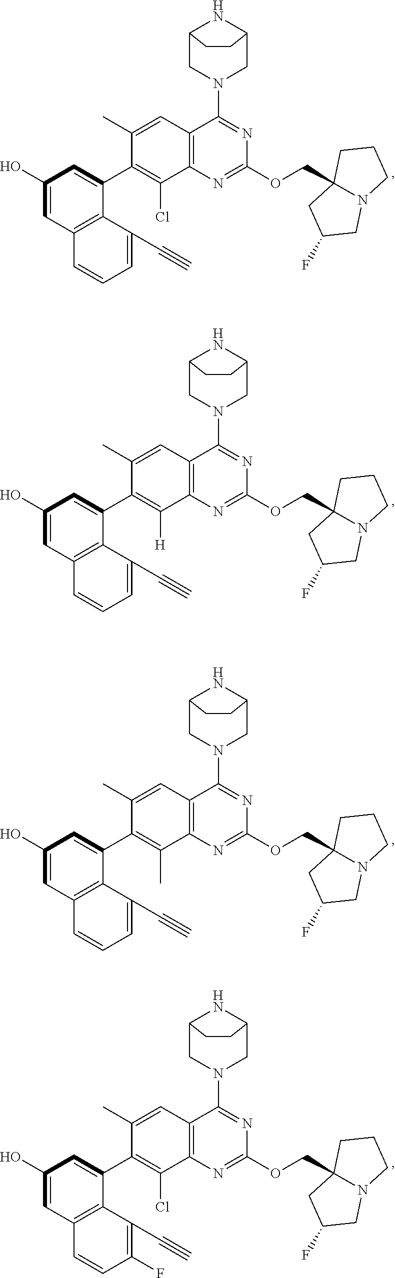

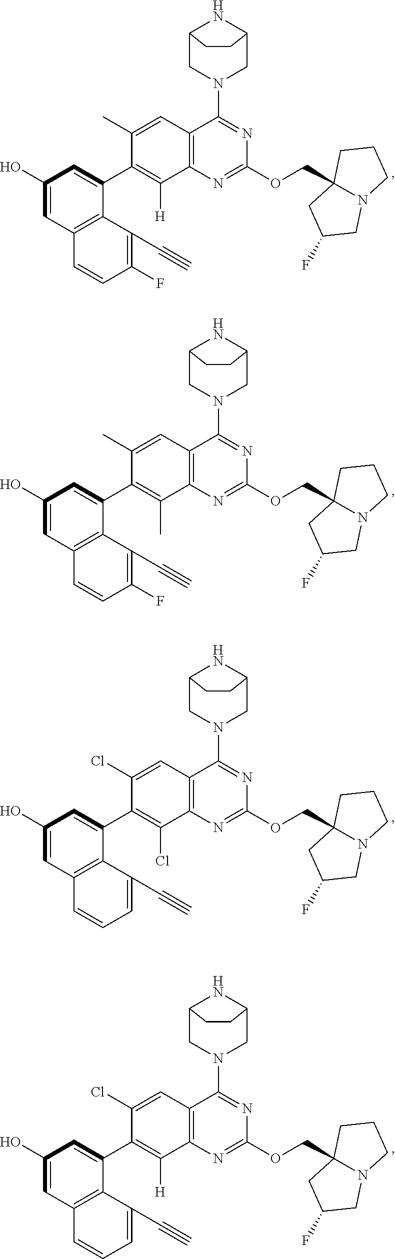

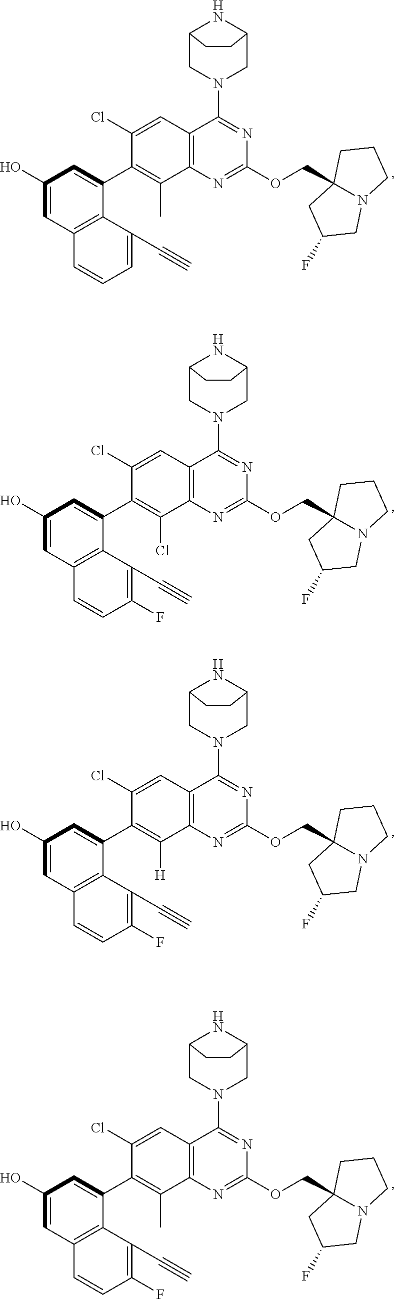

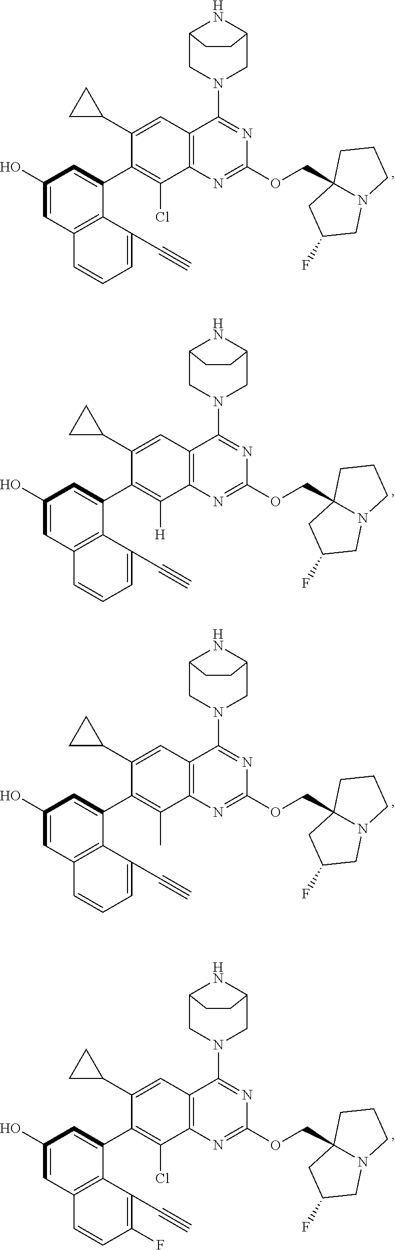

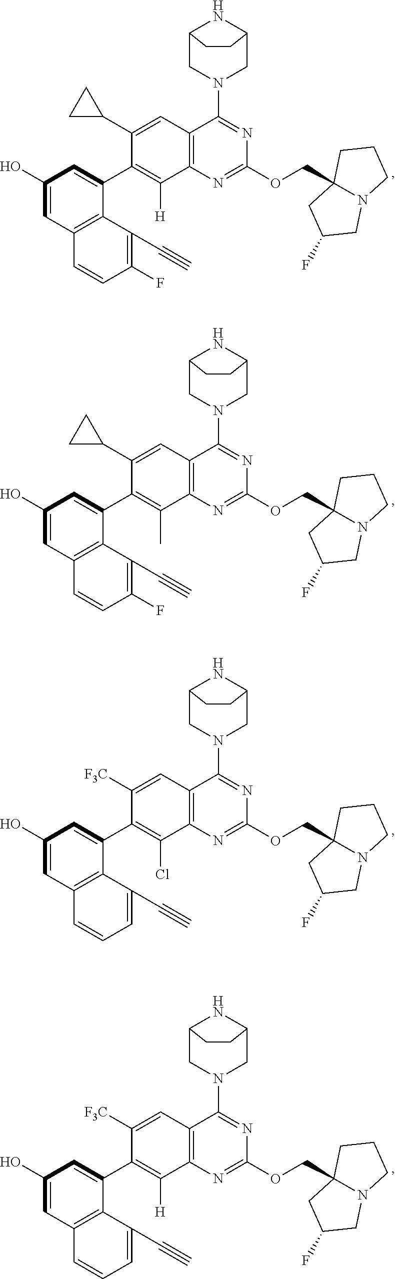

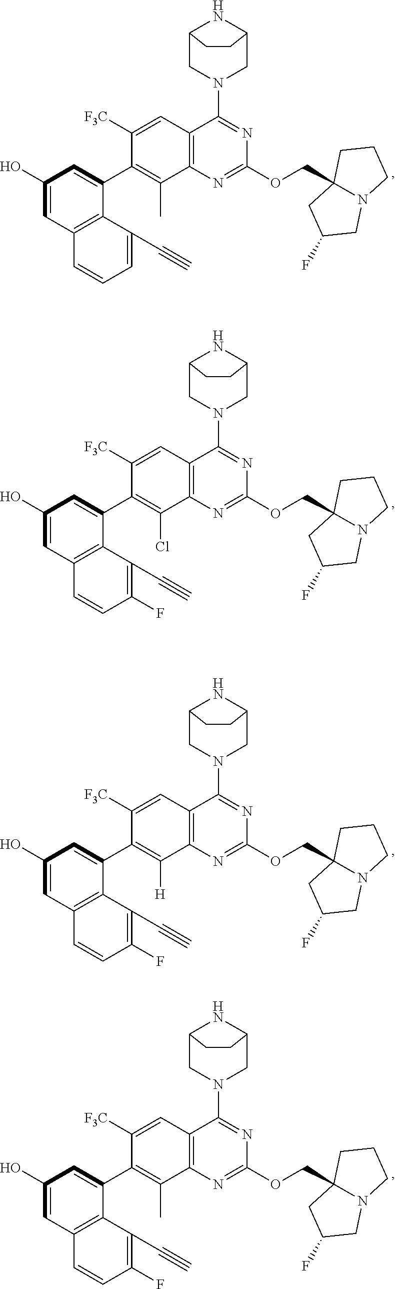

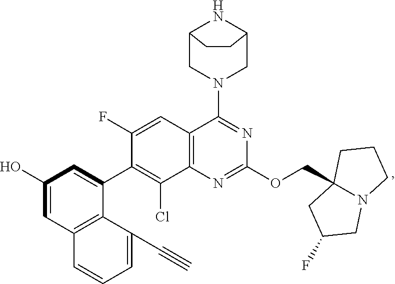

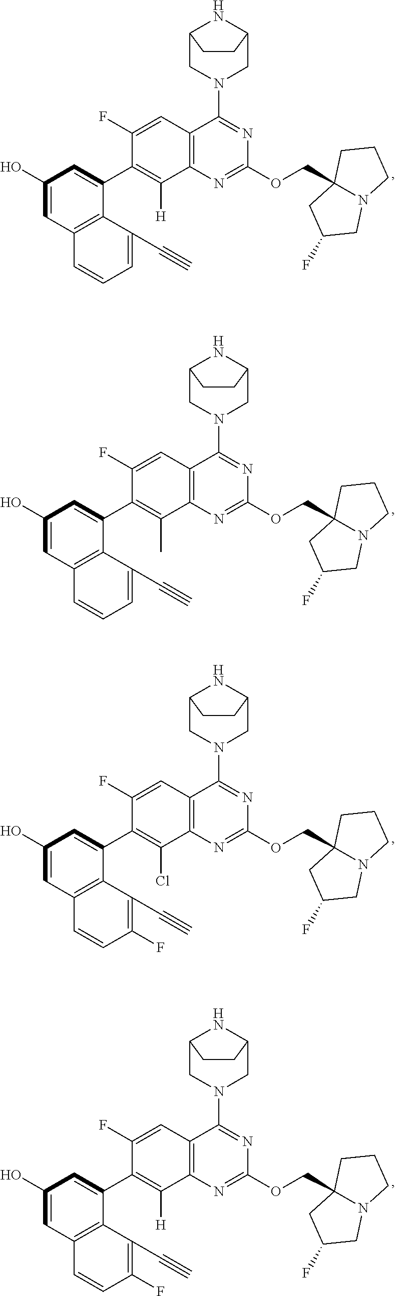

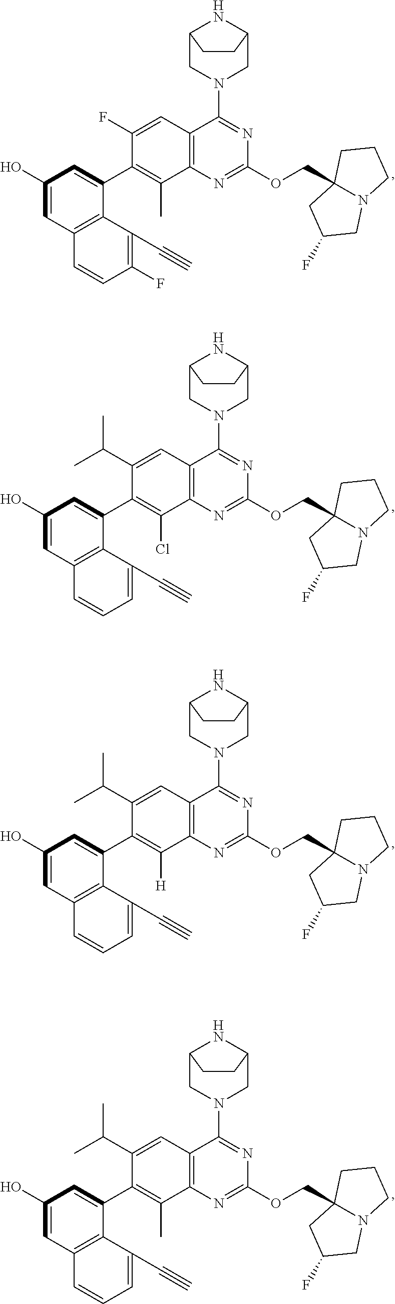

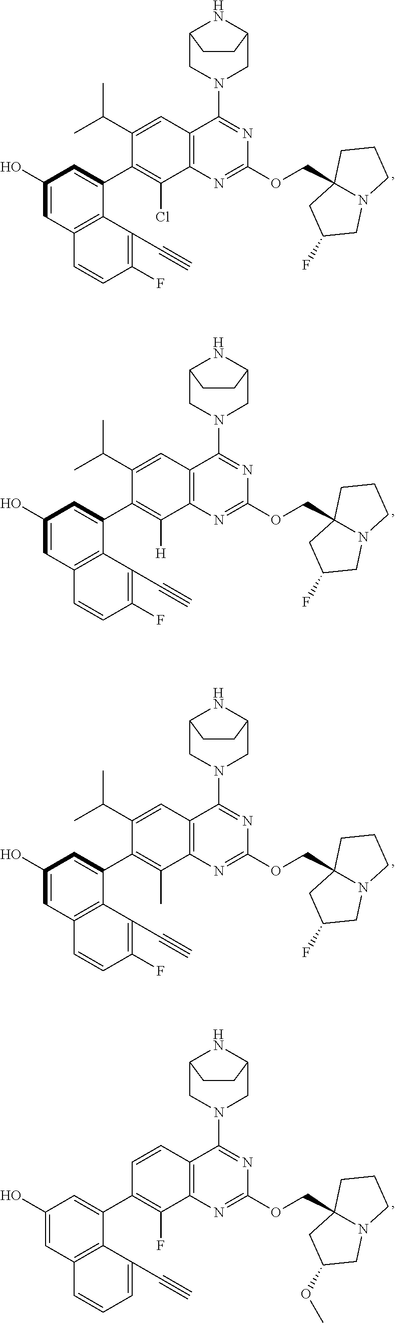

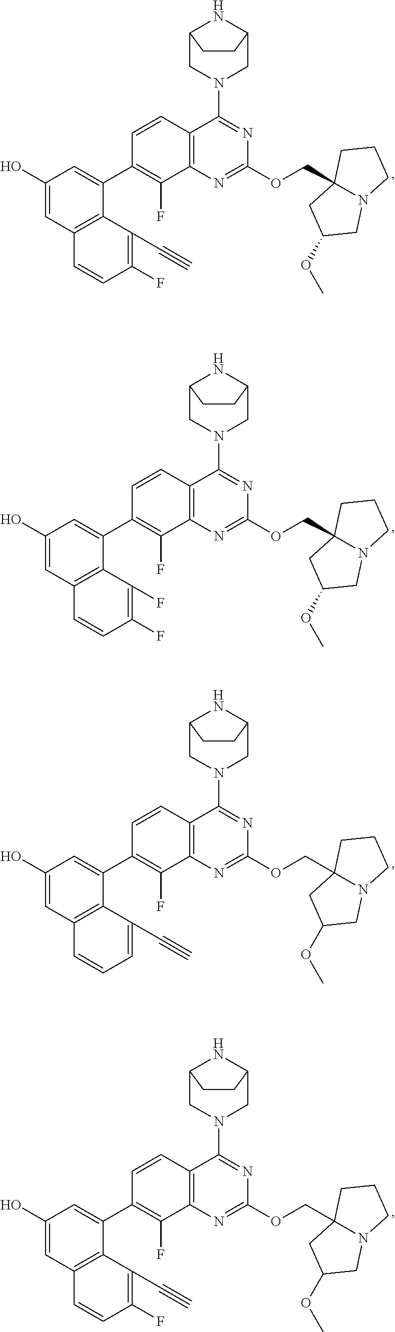 [SMILES: C#Cc1c(F)ccc2cc(O)cc(-c3ccc4c(N5CC6CCC(C5)N6)nc(OC[C@@]56CCCN5C[C@H](OC)C6)nc4c3F)c12 ; CO[C@H]1CN2CCC[C@@]2(COc2nc(N3CC4CCC(C3)N4)c3ccc(-c4cc(O)cc5ccc(F)c(F)c45)c(F)c3n2)C1 ; C#Cc1cccc2cc(O)cc(-c3ccc4c(N5CC6CCC(C5)N6)nc(OCC56CCCN5CC(OC)C6)nc4c3F)c12 ; C#Cc1c(F)ccc2cc(O)cc(-c3ccc4c(N5CC6CCC(C5)N6)nc(OCC56CCCN5CC(OC)C6)nc4c3F)c12]

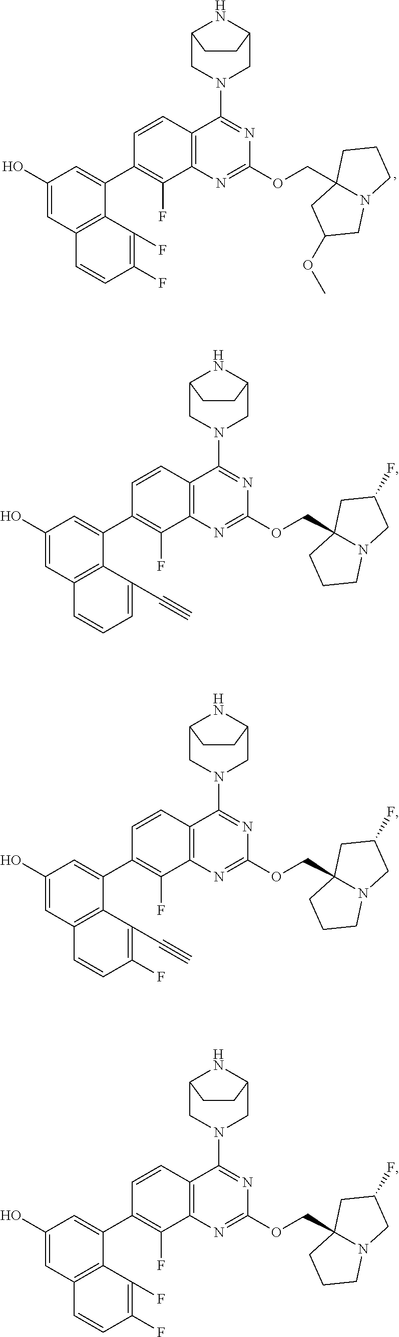 [SMILES: COC1CN2CCCC2(COc2nc(N3CC4CCC(C3)N4)c3ccc(-c4cc(O)cc5ccc(F)c(F)c45)c(F)c3n2)C1 ; C#Cc1cccc2cc(O)cc(-c3ccc4c(N5CC6CCC(C5)N6)nc(OC[C@]56CCCN5C[C@@H](F)C6)nc4c3F)c12 ; C#Cc1c(F)ccc2cc(O)cc(-c3ccc4c(N5CC6CCC(C5)N6)nc(OC[C@]56CCCN5C[C@@H](F)C6)nc4c3F)c12 ; Oc1cc(-c2ccc3c(N4CC5CCC(C4)N5)nc(OC[C@]45CCCN4C[C@@H](F)C5)nc3c2F)c2c(F)c(F)ccc2c1]

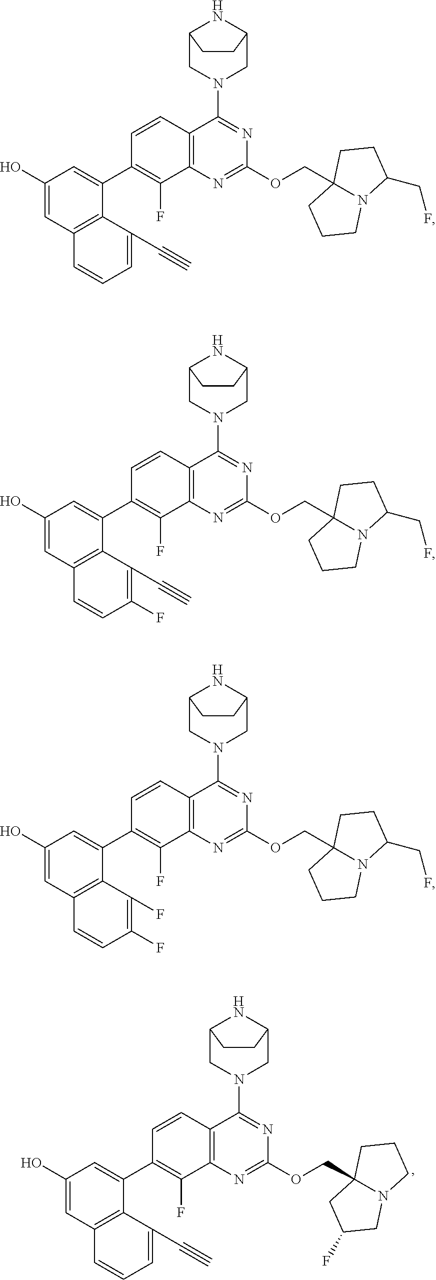 [SMILES: C#Cc1cccc2cc(O)cc(-c3ccc4c(N5CC6CCC(C5)N6)nc(OCC56CCCN5C(CF)CC6)nc4c3F)c12 ; C#Cc1c(F)ccc2cc(O)cc(-c3ccc4c(N5CC6CCC(C5)N6)nc(OCC56CCCN5C(CF)CC6)nc4c3F)c12 ; Oc1cc(-c2ccc3c(N4CC5CCC(C4)N5)nc(OCC45CCCN4C(CF)CC5)nc3c2F)c2c(F)c(F)ccc2c1 ; C#Cc1cccc2cc(O)cc(-c3ccc4c(N5CC6CCC(C5)N6)nc(OC[C@@]56CCCN5C[C@H](F)C6)nc4c3F)c12]

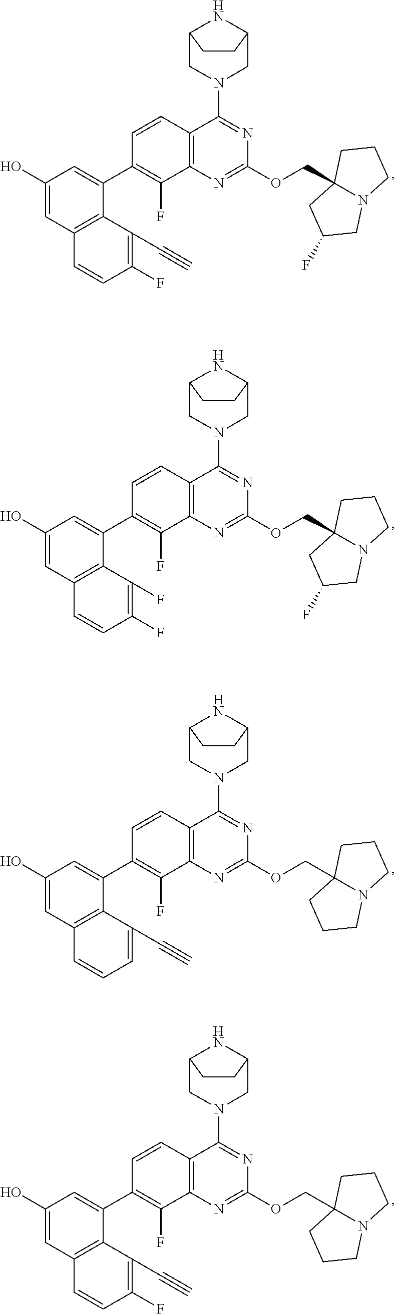 [SMILES: C#Cc1c(F)ccc2cc(O)cc(-c3ccc4c(N5CC6CCC(C5)N6)nc(OC[C@@]56CCCN5C[C@H](F)C6)nc4c3F)c12 ; Oc1cc(-c2ccc3c(N4CC5CCC(C4)N5)nc(OC[C@@]45CCCN4C[C@H](F)C5)nc3c2F)c2c(F)c(F)ccc2c1 ; C#Cc1cccc2cc(O)cc(-c3ccc4c(N5CC6CCC(C5)N6)nc(OCC56CCCN5CCC6)nc4c3F)c12 ; C#Cc1c(F)ccc2cc(O)cc(-c3ccc4c(N5CC6CCC(C5)N6)nc(OCC56CCCN5CCC6)nc4c3F)c12]

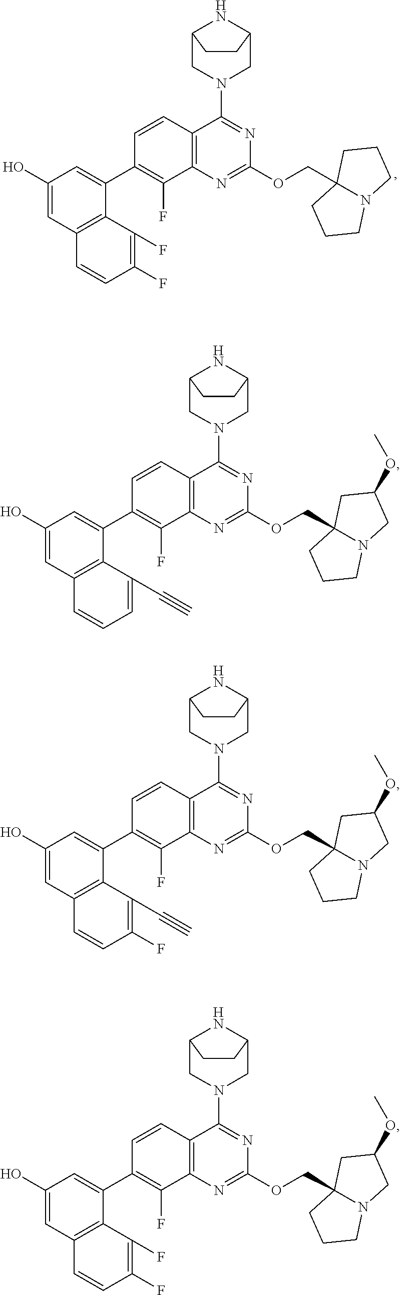 [SMILES: Oc1cc(-c2ccc3c(N4CC5CCC(C4)N5)nc(OCC45CCCN4CCC5)nc3c2F)c2c(F)c(F)ccc2c1 ; C#Cc1cccc2cc(O)cc(-c3ccc4c(N5CC6CCC(C5)N6)nc(OC[C@]56CCCN5C[C@H](OC)C6)nc4c3F)c12 ; C#Cc1c(F)ccc2cc(O)cc(-c3ccc4c(N5CC6CCC(C5)N6)nc(OC[C@]56CCCN5C[C@H](OC)C6)nc4c3F)c12 ; CO[C@H]1CN2CCC[C@]2(COc2nc(N3CC4CCC(C3)N4)c3ccc(-c4cc(O)cc5ccc(F)c(F)c45)c(F)c3n2)C1]

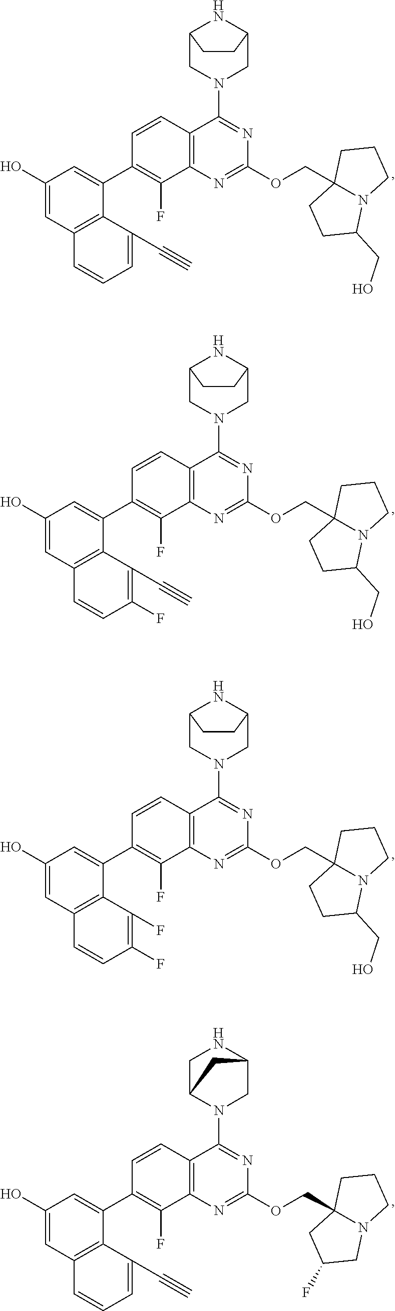 [SMILES: C#Cc1cccc2cc(O)cc(-c3ccc4c(N5CC6CCC(C5)N6)nc(OCC56CCCN5C(CO)CC6)nc4c3F)c12 ; C#Cc1c(F)ccc2cc(O)cc(-c3ccc4c(N5CC6CCC(C5)N6)nc(OCC56CCCN5C(CO)CC6)nc4c3F)c12 ; OCC1CCC2(COc3nc(N4CC5CCC(C4)N5)c4ccc(-c5cc(O)cc6ccc(F)c(F)c56)c(F)c4n3)CCCN12 ; C#Cc1cccc2cc(O)cc(-c3ccc4c(N5C[C@@H]6C[C@H]5CN6)nc(OC[C@@]56CCCN5C[C@H](F)C6)nc4c3F)c12]

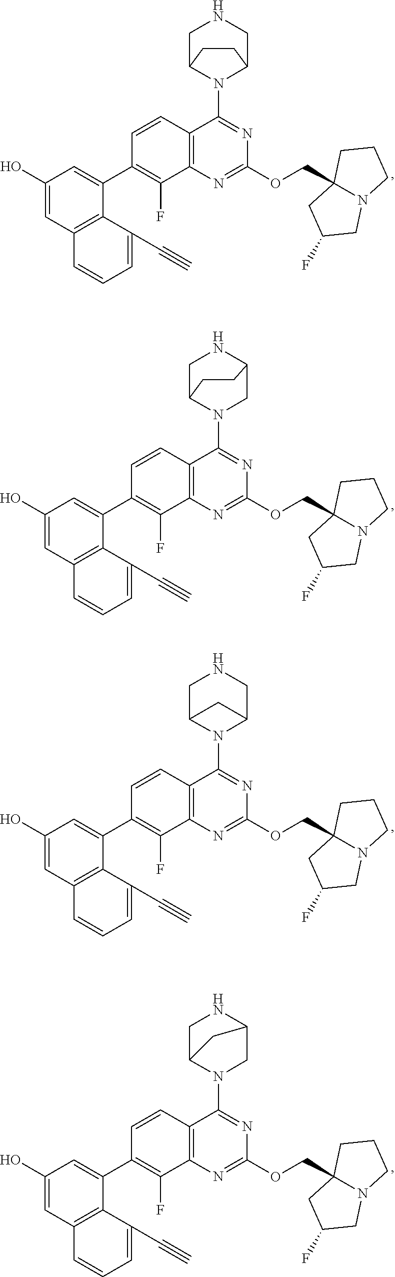 [SMILES: C#Cc1cccc2cc(O)cc(-c3ccc4c(N5C6CCC5CNC6)nc(OC[C@@]56CCCN5C[C@H](F)C6)nc4c3F)c12 ; C#Cc1cccc2cc(O)cc(-c3ccc4c(N5CC6CCC5CN6)nc(OC[C@@]56CCCN5C[C@H](F)C6)nc4c3F)c12 ; C#Cc1cccc2cc(O)cc(-c3ccc4c(N5C6CNCC5C6)nc(OC[C@@]56CCCN5C[C@H](F)C6)nc4c3F)c12 ; C#Cc1cccc2cc(O)cc(-c3ccc4c(N5CC6CC5CN6)nc(OC[C@@]56CCCN5C[C@H](F)C6)nc4c3F)c12]

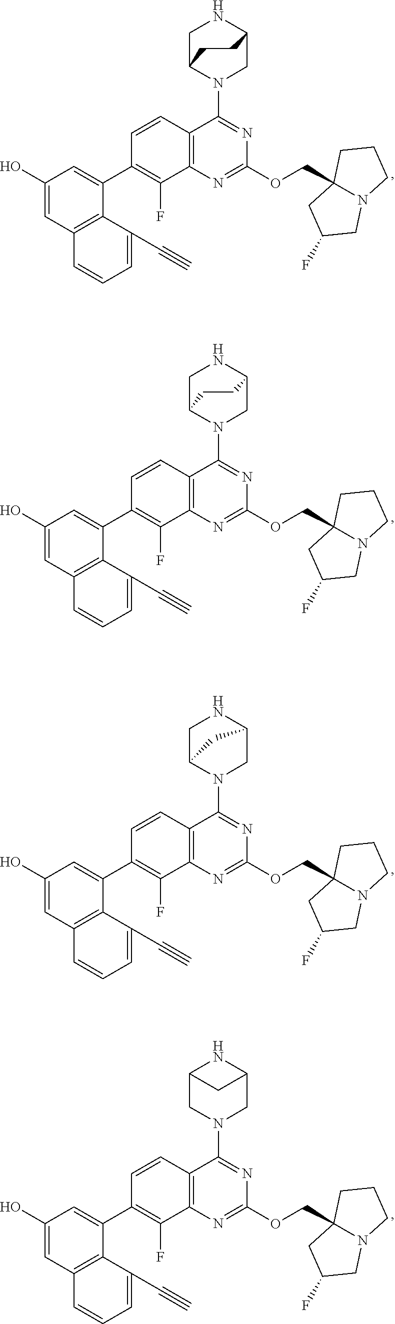 [SMILES: C#Cc1cccc2cc(O)cc(-c3ccc4c(N5C[C@@H]6CC[C@H]5CN6)nc(OC[C@@]56CCCN5C[C@H](F)C6)nc4c3F)c12 ; C#Cc1cccc2cc(O)cc(-c3ccc4c(N5C[C@H]6CC[C@@H]5CN6)nc(OC[C@@]56CCCN5C[C@H](F)C6)nc4c3F)c12 ; C#Cc1cccc2cc(O)cc(-c3ccc4c(N5C[C@H]6C[C@@H]5CN6)nc(OC[C@@]56CCCN5C[C@H](F)C6)nc4c3F)c12 ; C#Cc1cccc2cc(O)cc(-c3ccc4c(N5CC6CC(C5)N6)nc(OC[C@@]56CCCN5C[C@H](F)C6)nc4c3F)c12]

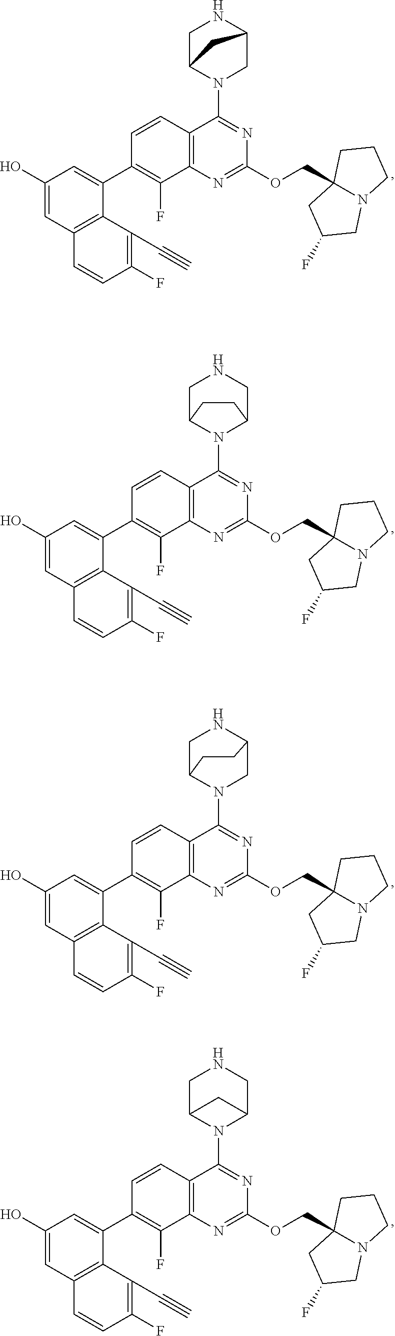 [SMILES: C#Cc1c(F)ccc2cc(O)cc(-c3ccc4c(N5C[C@@H]6C[C@H]5CN6)nc(OC[C@@]56CCCN5C[C@H](F)C6)nc4c3F)c12 ; C#Cc1c(F)ccc2cc(O)cc(-c3ccc4c(N5C6CCC5CNC6)nc(OC[C@@]56CCCN5C[C@H](F)C6)nc4c3F)c12 ; C#Cc1c(F)ccc2cc(O)cc(-c3ccc4c(N5CC6CCC5CN6)nc(OC[C@@]56CCCN5C[C@H](F)C6)nc4c3F)c12 ; C#Cc1c(F)ccc2cc(O)cc(-c3ccc4c(N5C6CNCC5C6)nc(OC[C@@]56CCCN5C[C@H](F)C6)nc4c3F)c12]

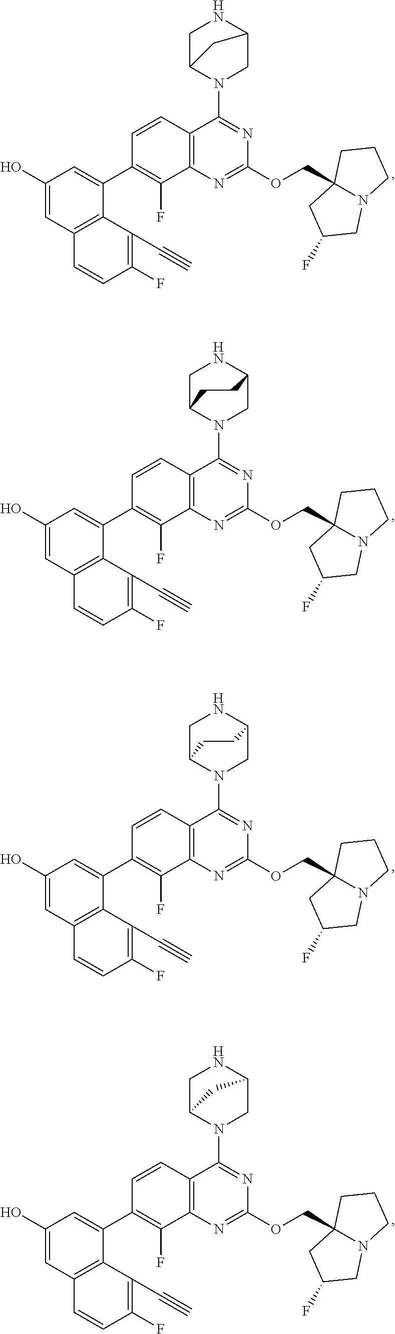 [SMILES: C#Cc1c(F)ccc2cc(O)cc(-c3ccc4c(N5CC6CC5CN6)nc(OC[C@@]56CCCN5C[C@H](F)C6)nc4c3F)c12 ; C#Cc1c(F)ccc2cc(O)cc(-c3ccc4c(N5C[C@@H]6CC[C@H]5CN6)nc(OC[C@@]56CCCN5C[C@H](F)C6)nc4c3F)c12 ; C#Cc1c(F)ccc2cc(O)cc(-c3ccc4c(N5C[C@H]6CC[C@@H]5CN6)nc(OC[C@@]56CCCN5C[C@H](F)C6)nc4c3F)c12 ; C#Cc1c(F)ccc2cc(O)cc(-c3ccc4c(N5C[C@H]6C[C@@H]5CN6)nc(OC[C@@]56CCCN5C[C@H](F)C6)nc4c3F)c12]

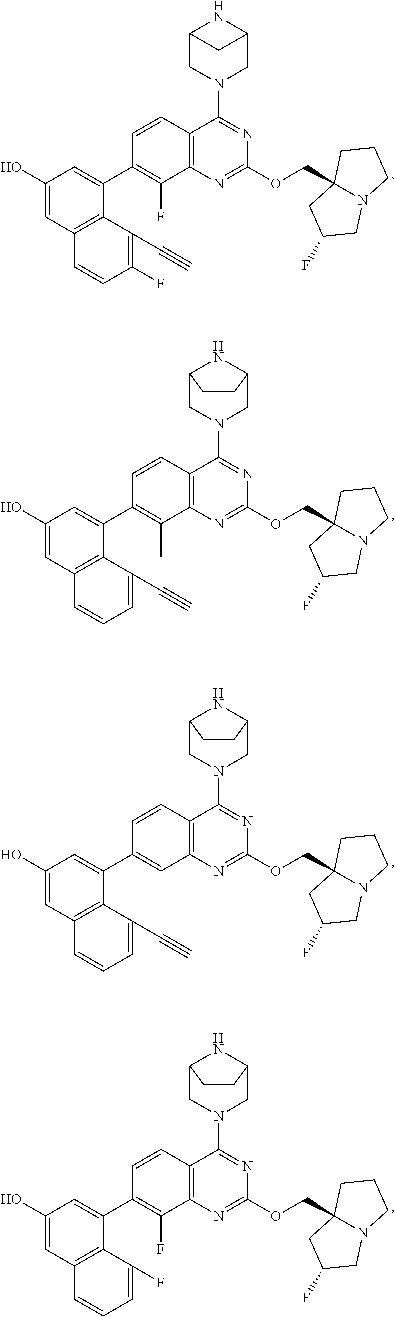 [SMILES: C#Cc1c(F)ccc2cc(O)cc(-c3ccc4c(N5CC6CC(C5)N6)nc(OC[C@@]56CCCN5C[C@H](F)C6)nc4c3F)c12 ; C#Cc1cccc2cc(O)cc(-c3ccc4c(N5CC6CCC(C5)N6)nc(OC[C@@]56CCCN5C[C@H](F)C6)nc4c3C)c12 ; C#Cc1cccc2cc(O)cc(-c3ccc4c(N5CC6CCC(C5)N6)nc(OC[C@@]56CCCN5C[C@H](F)C6)nc4c3)c12 ; Oc1cc(-c2ccc3c(N4CC5CCC(C4)N5)nc(OC[C@@]45CCCN4C[C@H](F)C5)nc3c2F)c2c(F)cccc2c1]

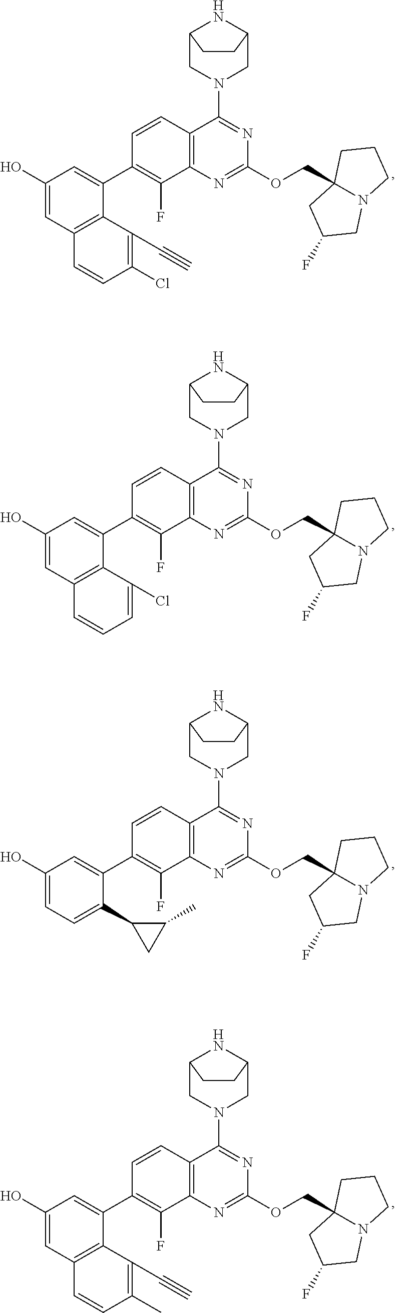 [SMILES: C#Cc1c(Cl)ccc2cc(O)cc(-c3ccc4c(N5CC6CCC(C5)N6)nc(OC[C@@]56CCCN5C[C@H](F)C6)nc4c3F)c12 ; Oc1cc(-c2ccc3c(N4CC5CCC(C4)N5)nc(OC[C@@]45CCCN4C[C@H](F)C5)nc3c2F)c2c(Cl)cccc2c1 ; C[C@@H]1C[C@H]1c1ccc(O)cc1-c1ccc2c(N3CC4CCC(C3)N4)nc(OC[C@@]34CCCN3C[C@H](F)C4)nc2c1F ; C#Cc1c(C)ccc2cc(O)cc(-c3ccc4c(N5CC6CCC(C5)N6)nc(OC[C@@]56CCCN5C[C@H](F)C6)nc4c3F)c12]

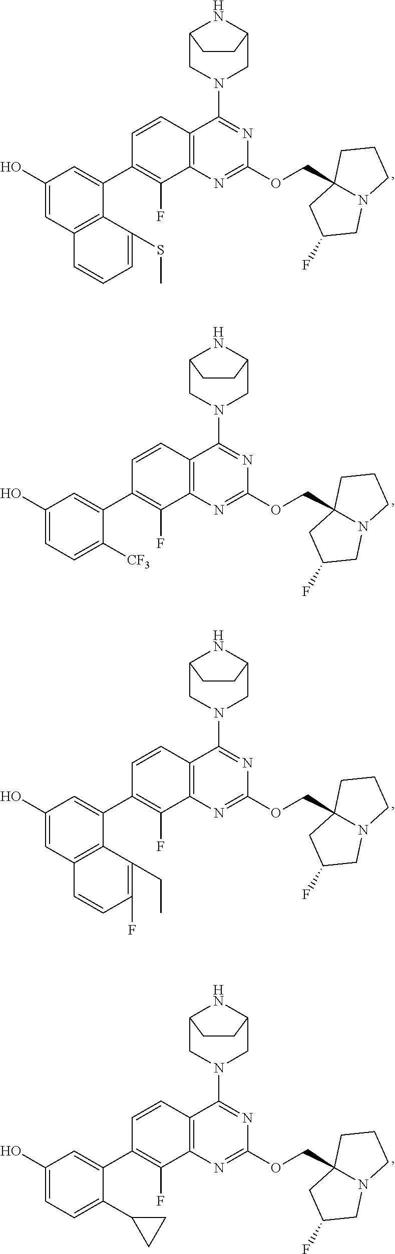 [SMILES: CSc1cccc2cc(O)cc(-c3ccc4c(N5CC6CCC(C5)N6)nc(OC[C@@]56CCCN5C[C@H](F)C6)nc4c3F)c12 ; Oc1ccc(C(F)(F)F)c(-c2ccc3c(N4CC5CCC(C4)N5)nc(OC[C@@]45CCCN4C[C@H](F)C5)nc3c2F)c1 ; CCc1c(F)ccc2cc(O)cc(-c3ccc4c(N5CC6CCC(C5)N6)nc(OC[C@@]56CCCN5C[C@H](F)C6)nc4c3F)c12 ; Oc1ccc(C2CC2)c(-c2ccc3c(N4CC5CCC(C4)N5)nc(OC[C@@]45CCCN4C[C@H](F)C5)nc3c2F)c1]

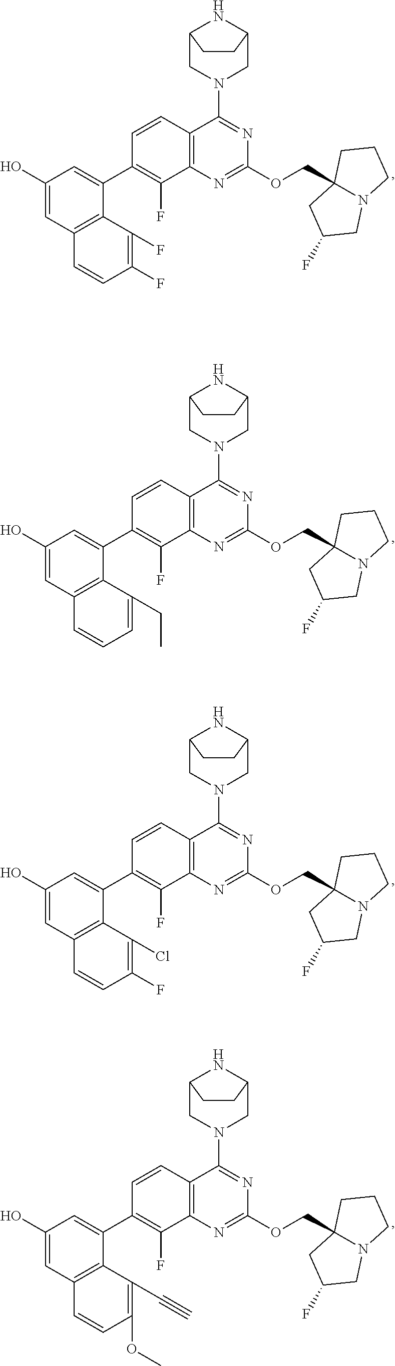 [SMILES: Oc1cc(-c2ccc3c(N4CC5CCC(C4)N5)nc(OC[C@@]45CCCN4C[C@H](F)C5)nc3c2F)c2c(F)c(F)ccc2c1 ; CCc1cccc2cc(O)cc(-c3ccc4c(N5CC6CCC(C5)N6)nc(OC[C@@]56CCCN5C[C@H](F)C6)nc4c3F)c12 ; Oc1cc(-c2ccc3c(N4CC5CCC(C4)N5)nc(OC[C@@]45CCCN4C[C@H](F)C5)nc3c2F)c2c(Cl)c(F)ccc2c1 ; C#Cc1c(OC)ccc2cc(O)cc(-c3ccc4c(N5CC6CCC(C5)N6)nc(OC[C@@]56CCCN5C[C@H](F)C6)nc4c3F)c12]

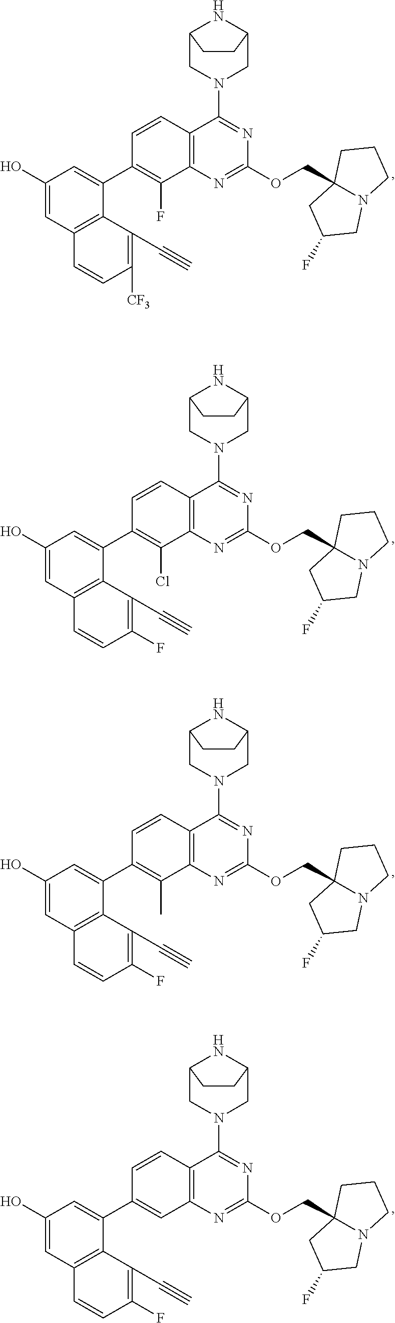 [SMILES: C#Cc1c(C(F)(F)F)ccc2cc(O)cc(-c3ccc4c(N5CC6CCC(C5)N6)nc(OC[C@@]56CCCN5C[C@H](F)C6)nc4c3F)c12 ; C#Cc1c(F)ccc2cc(O)cc(-c3ccc4c(N5CC6CCC(C5)N6)nc(OC[C@@]56CCCN5C[C@H](F)C6)nc4c3Cl)c12 ; C#Cc1c(F)ccc2cc(O)cc(-c3ccc4c(N5CC6CCC(C5)N6)nc(OC[C@@]56CCCN5C[C@H](F)C6)nc4c3C)c12 ; C#Cc1c(F)ccc2cc(O)cc(-c3ccc4c(N5CC6CCC(C5)N6)nc(OC[C@@]56CCCN5C[C@H](F)C6)nc4c3)c12]

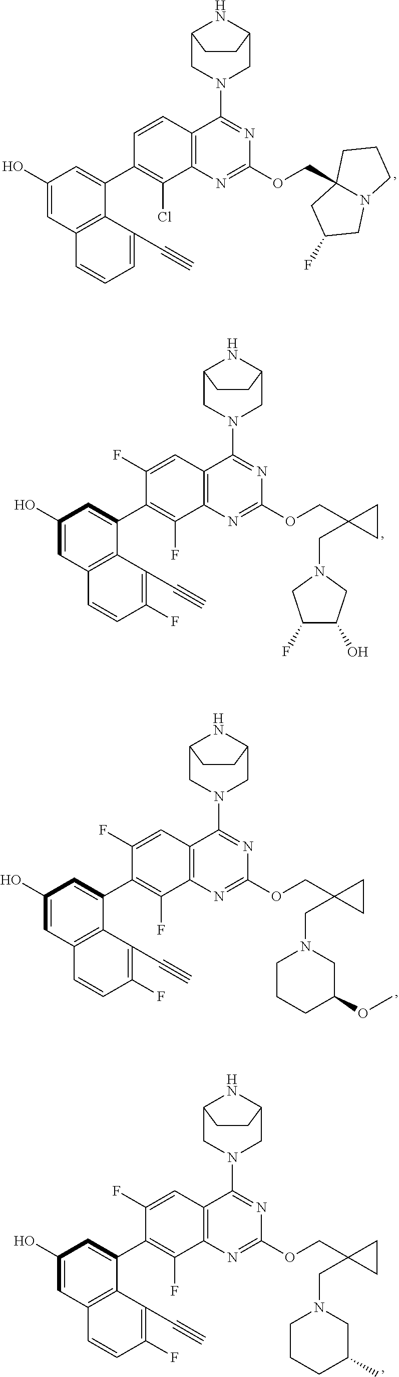

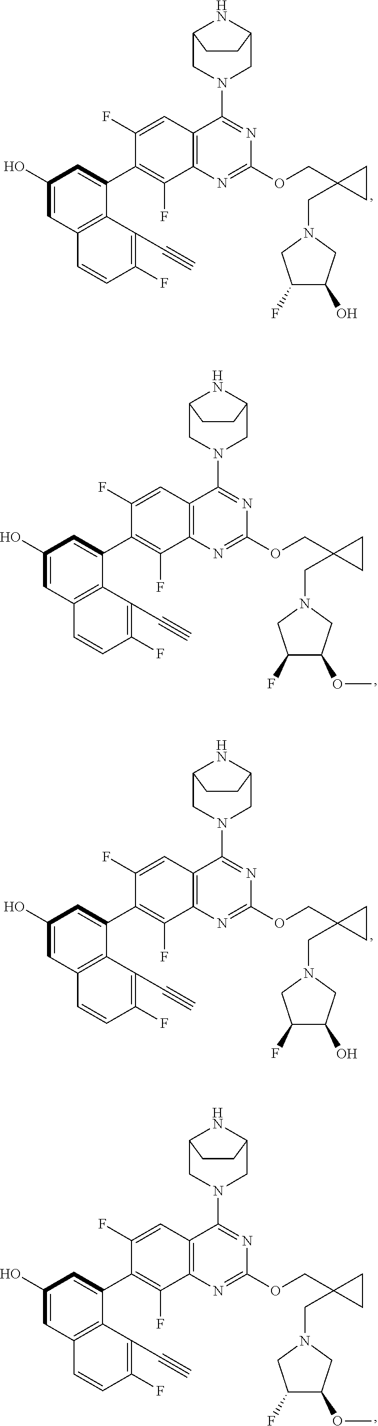

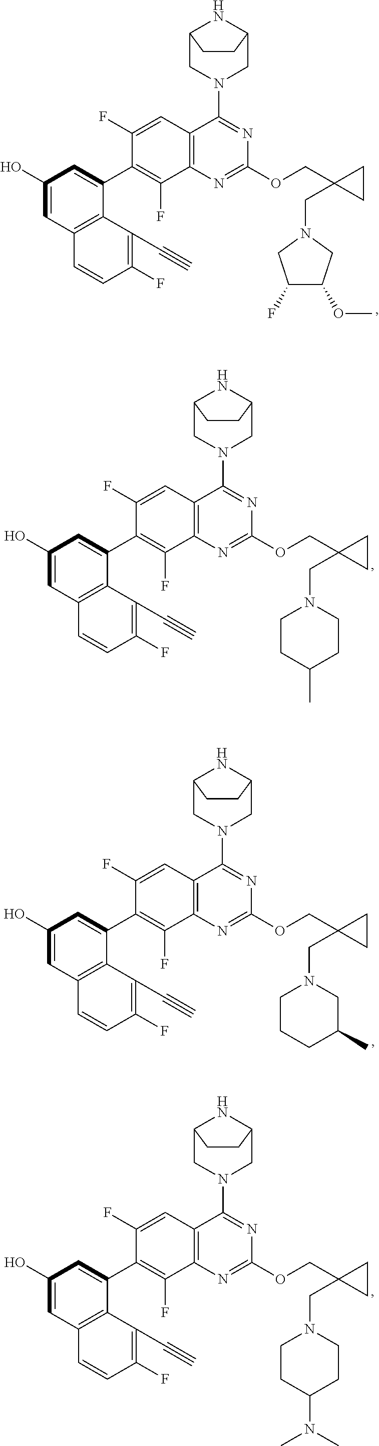

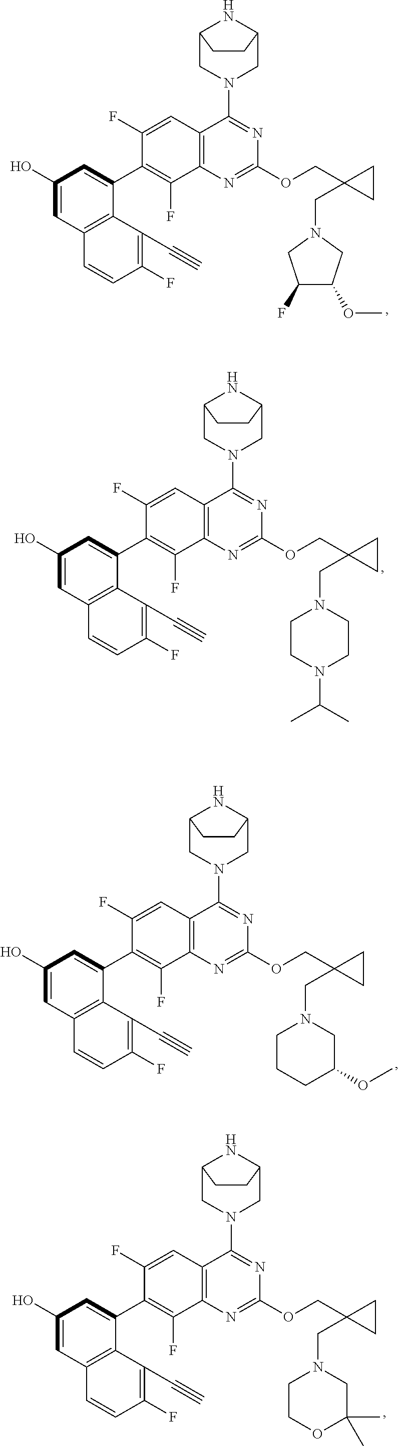

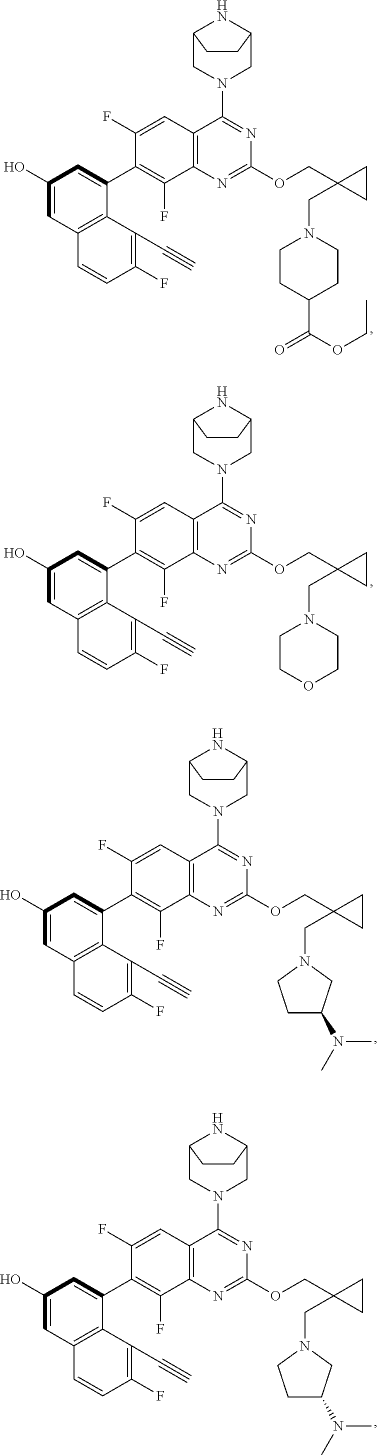

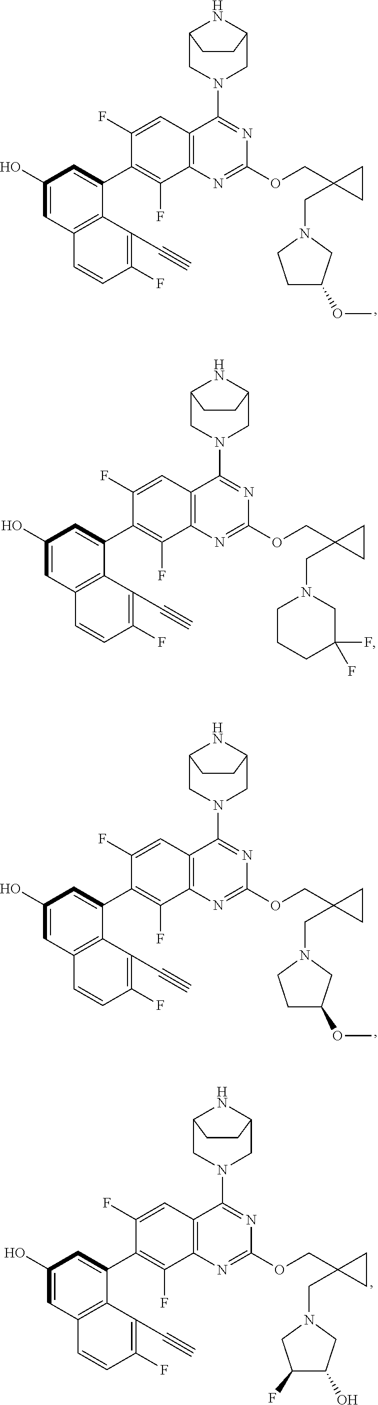

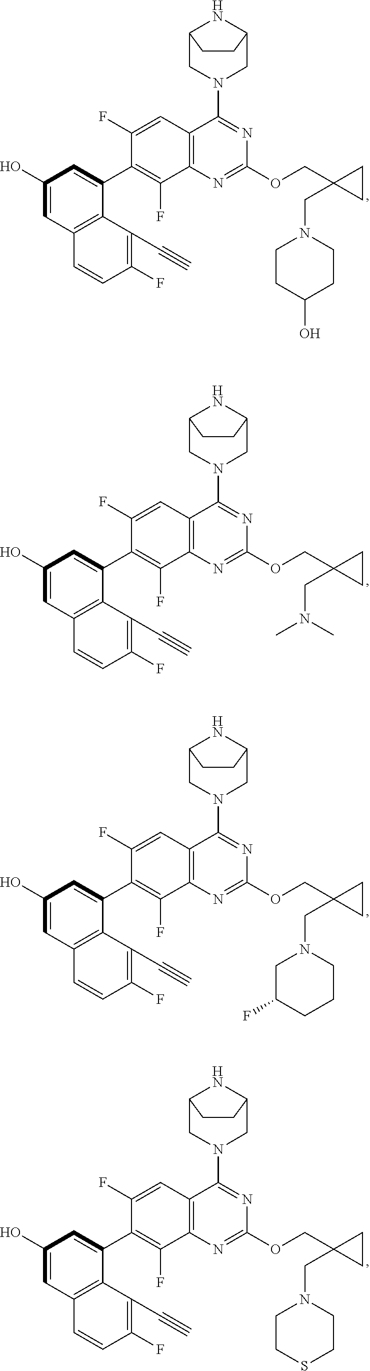

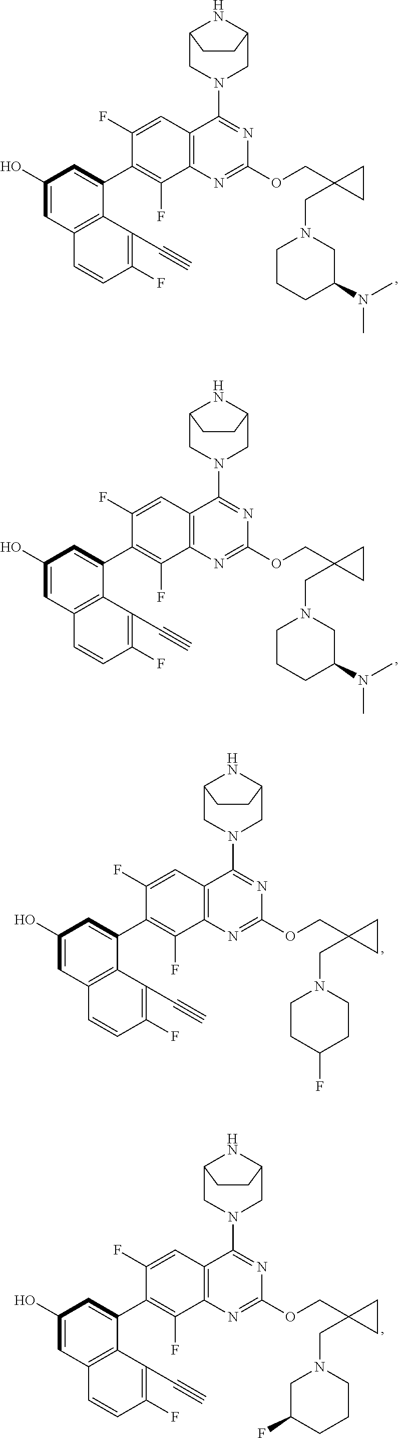

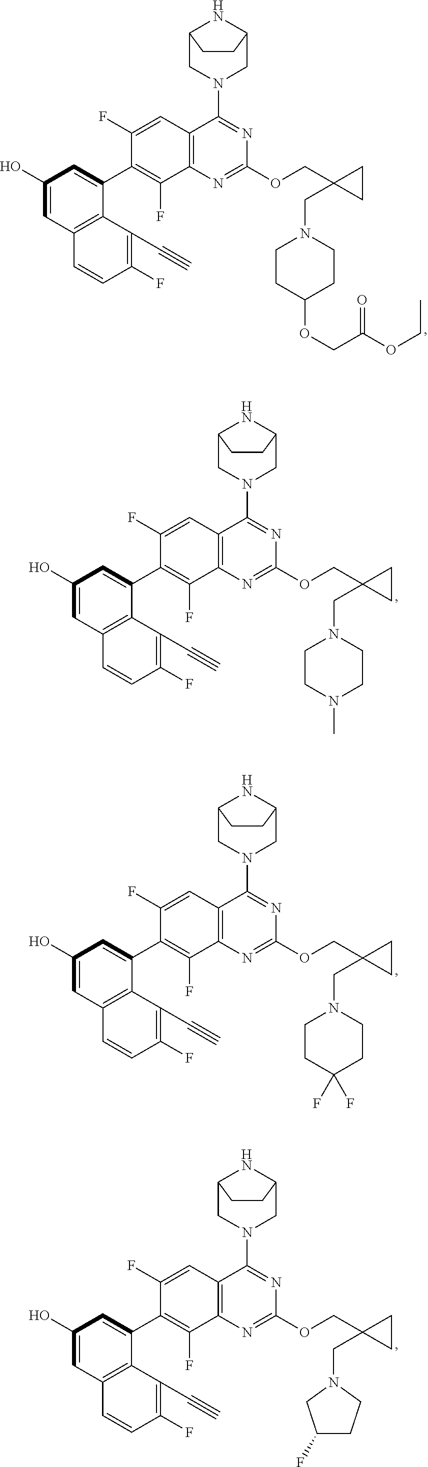

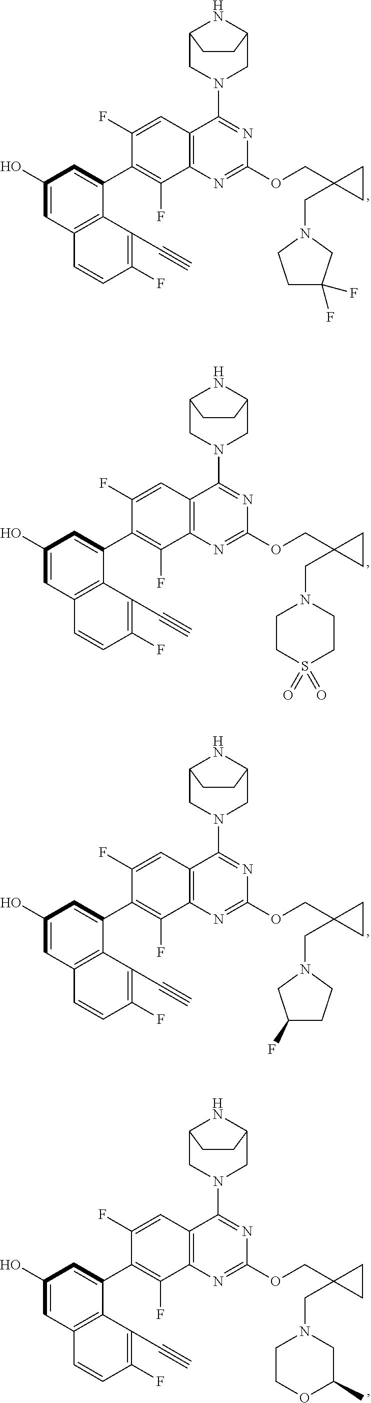

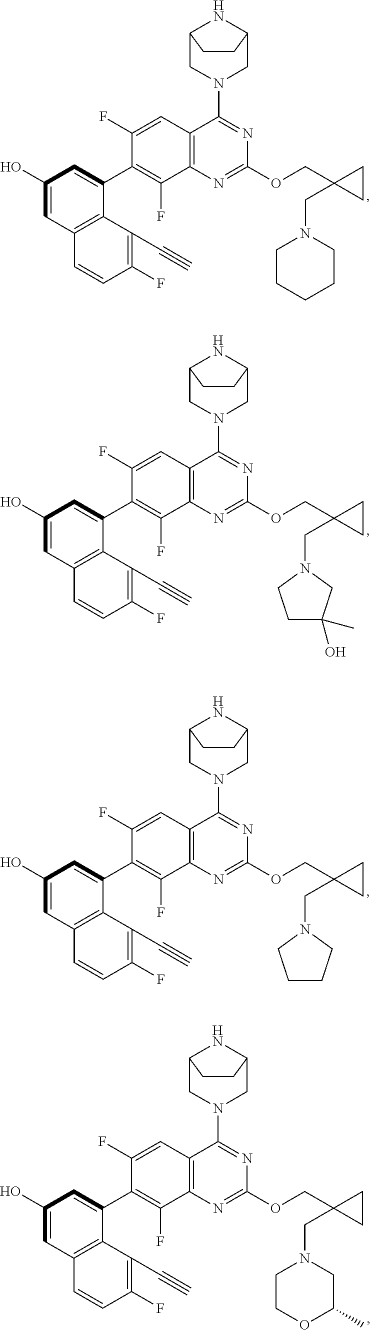

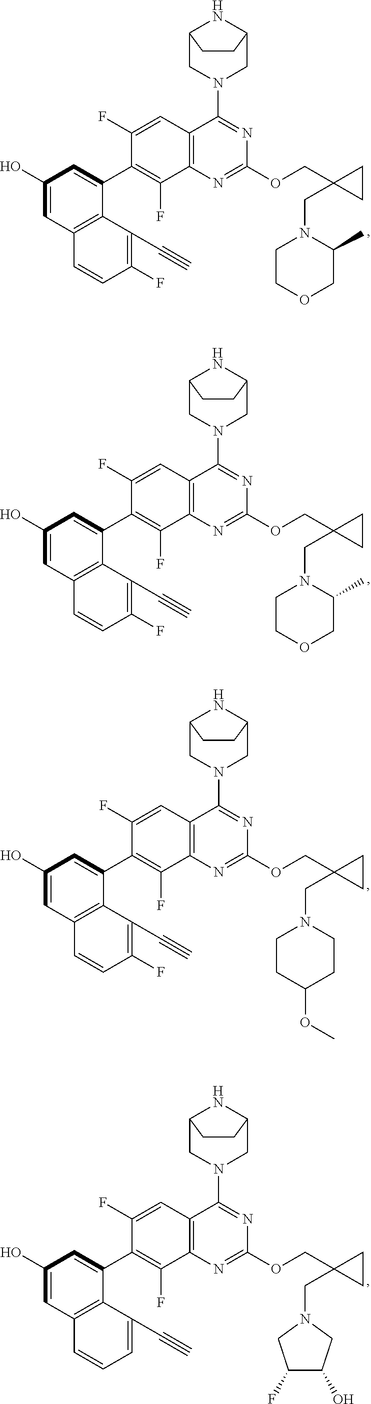

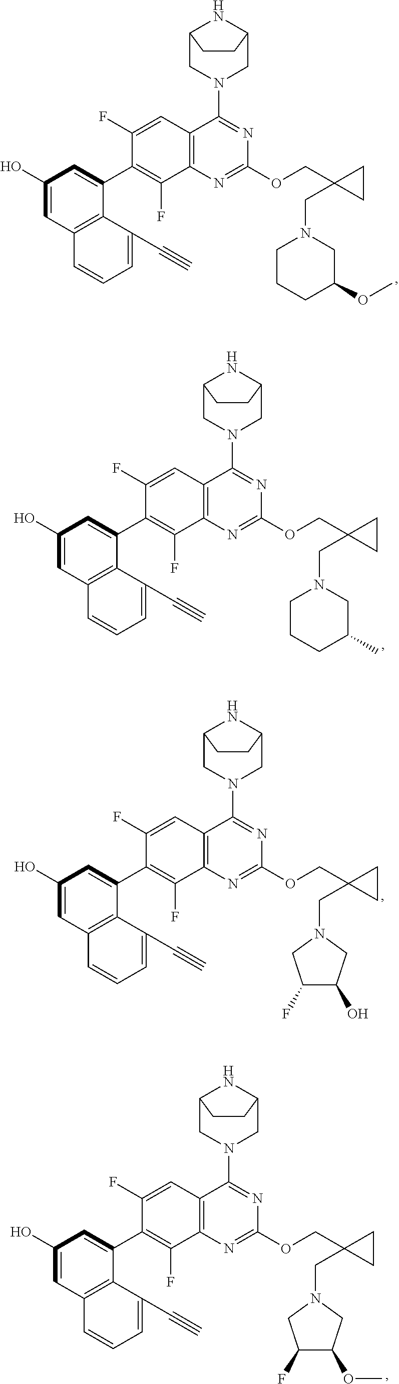

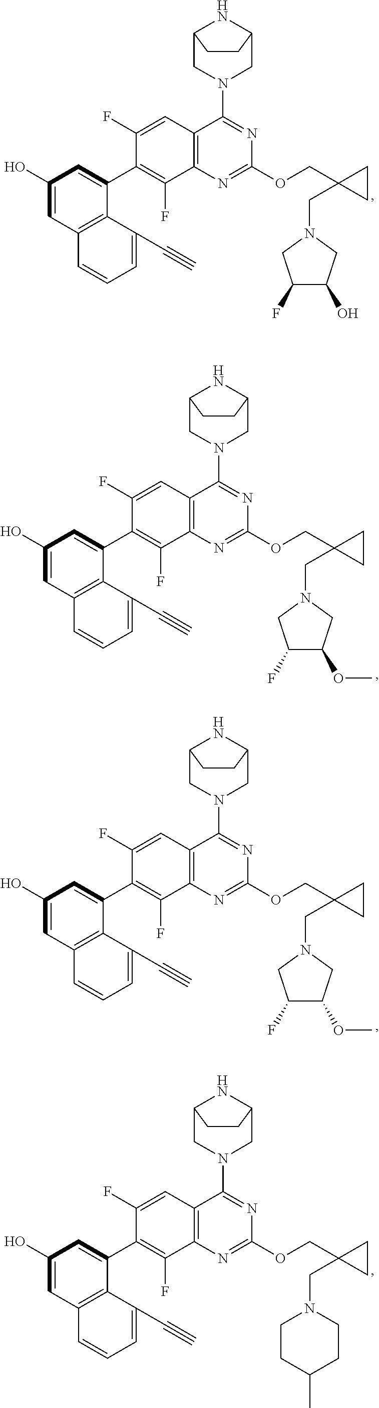

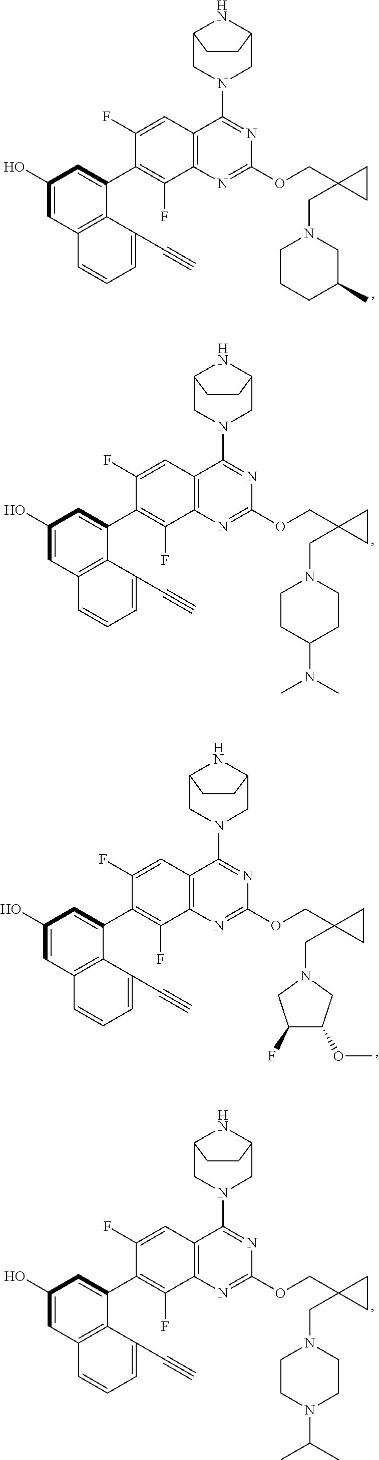

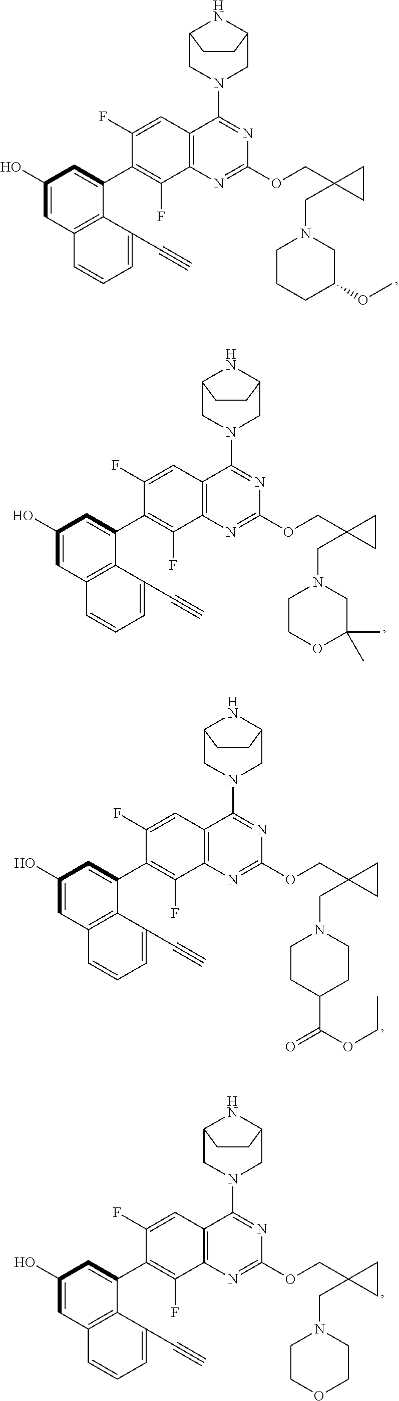

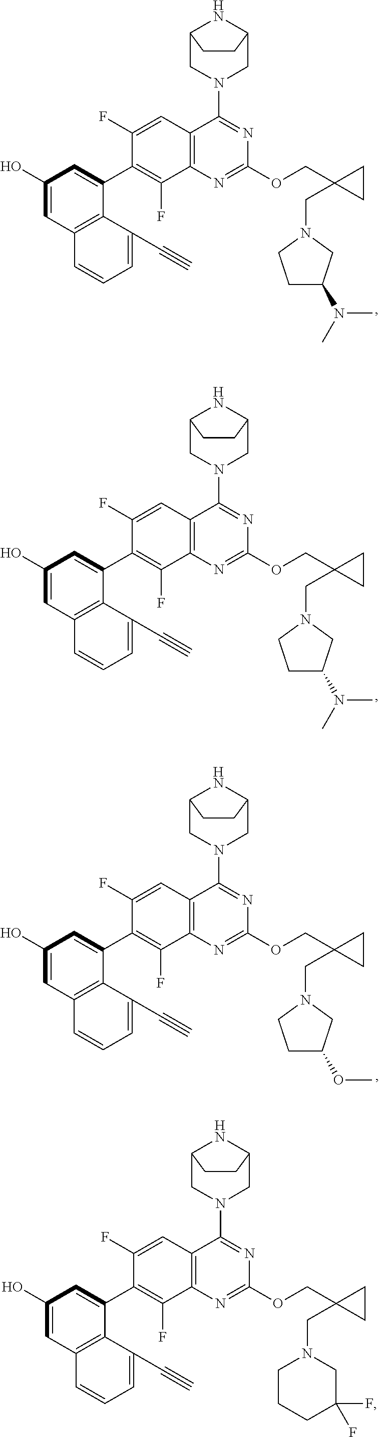

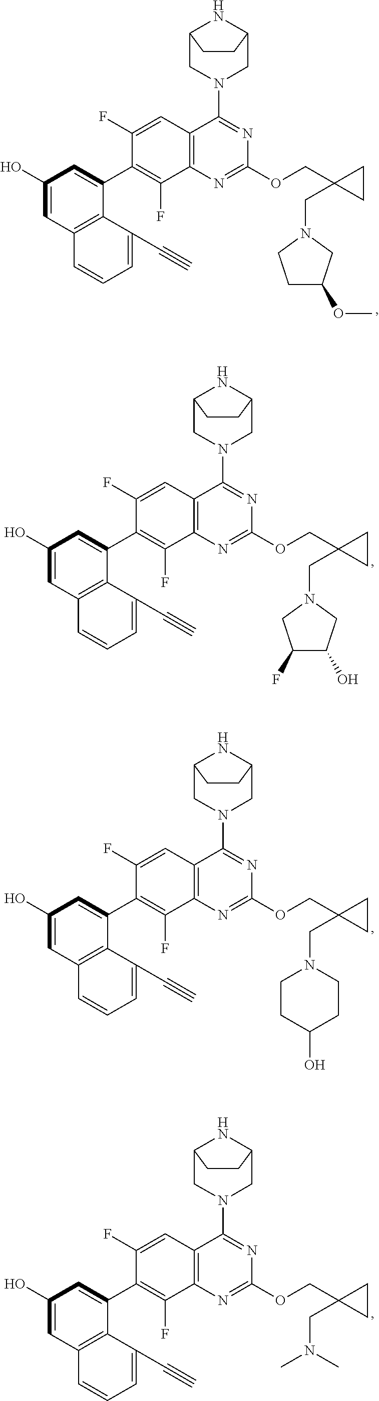

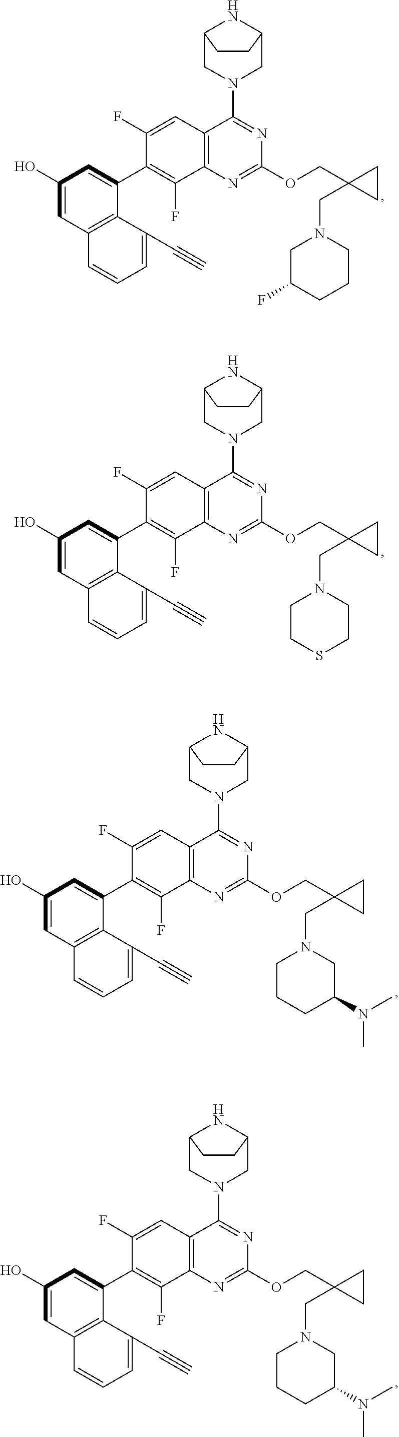

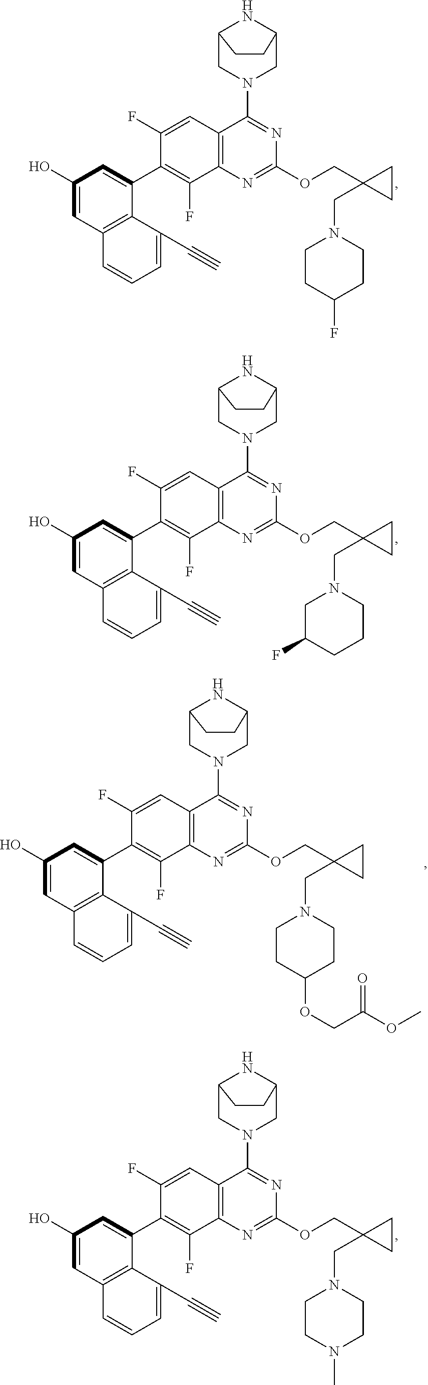

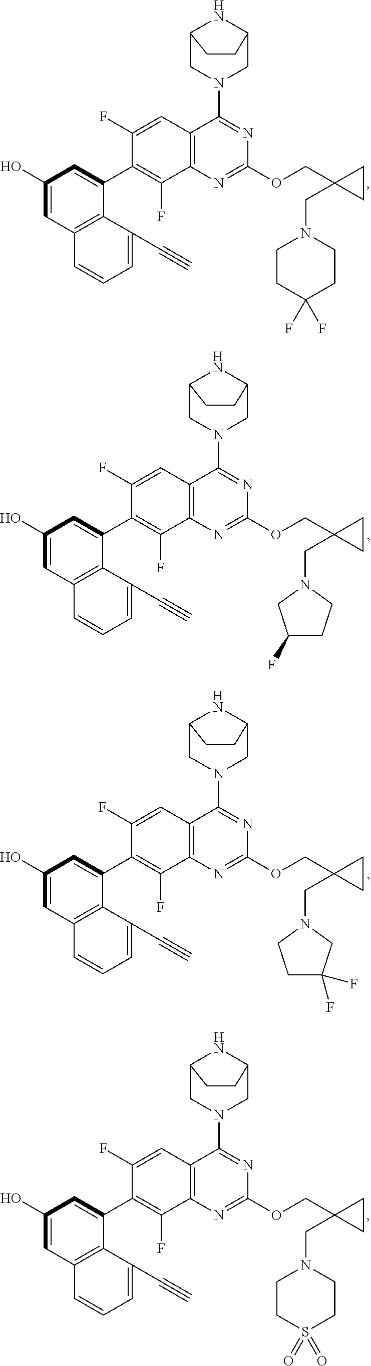

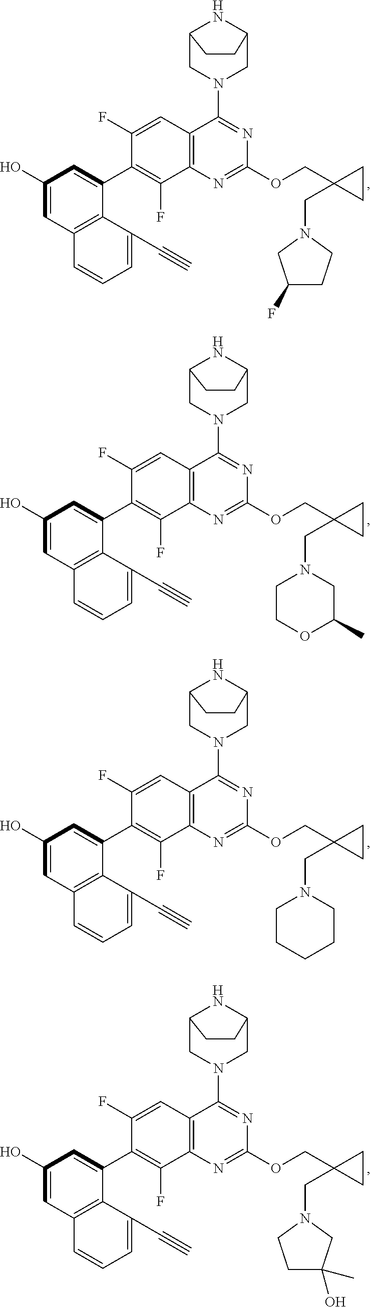

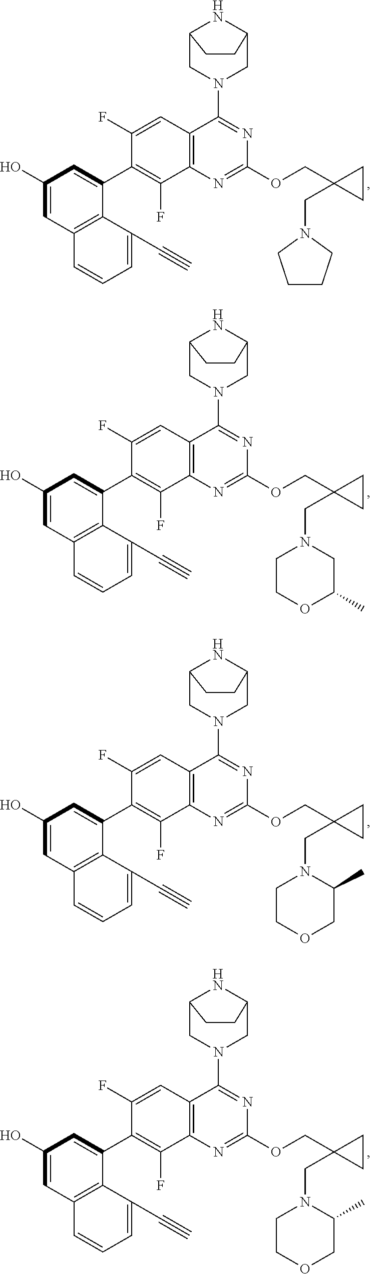

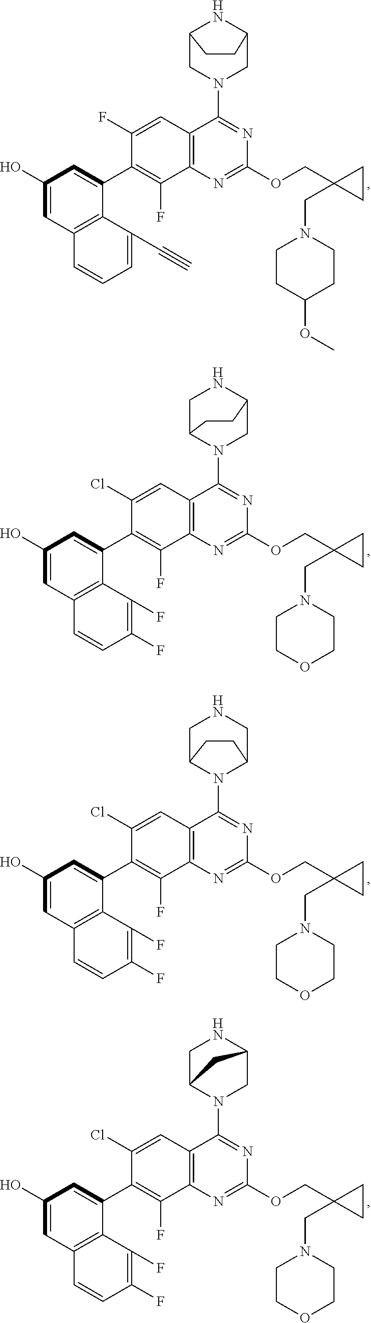

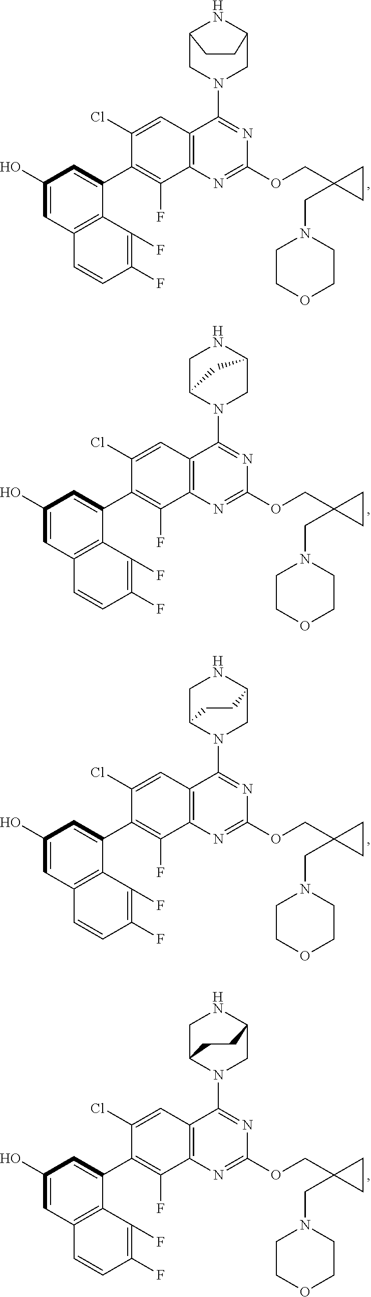

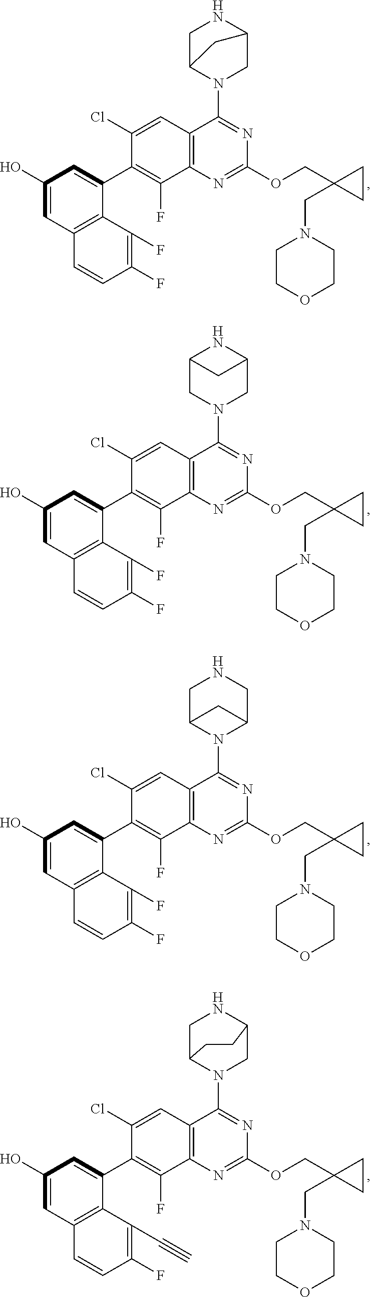

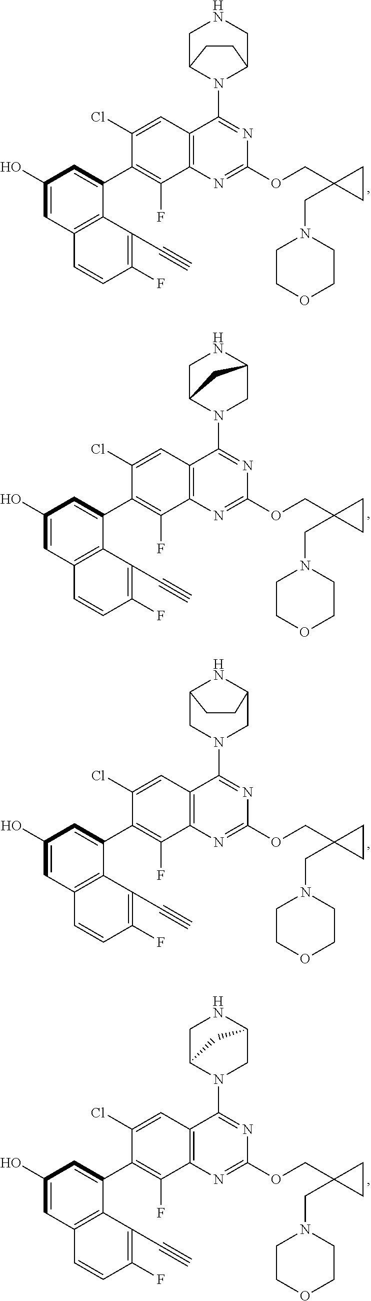

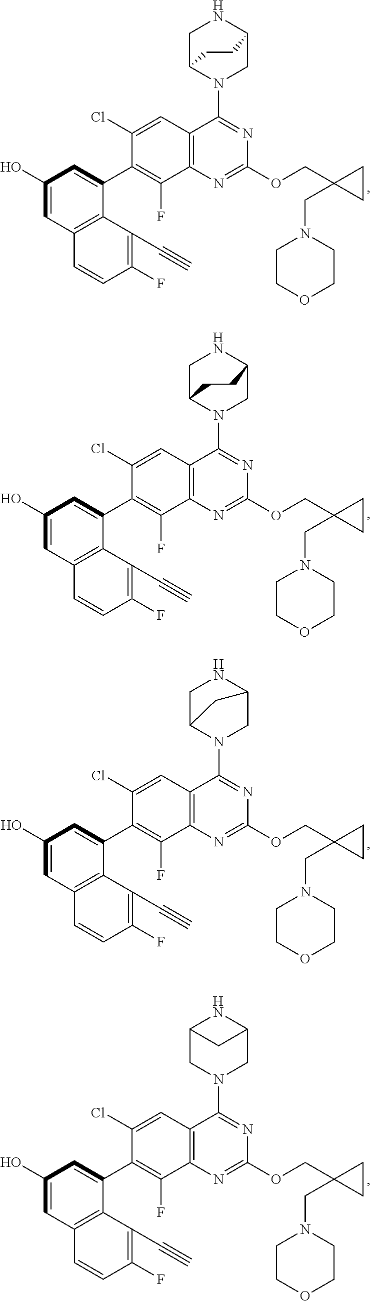

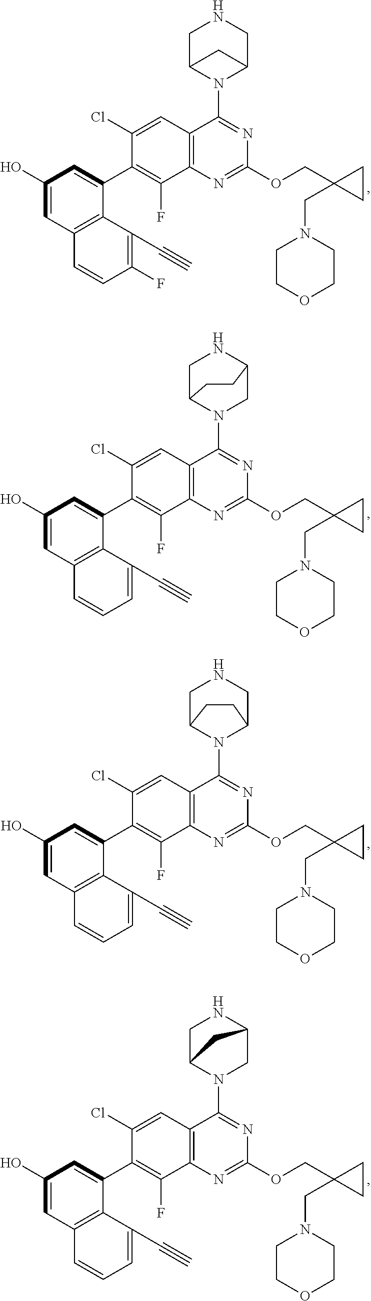

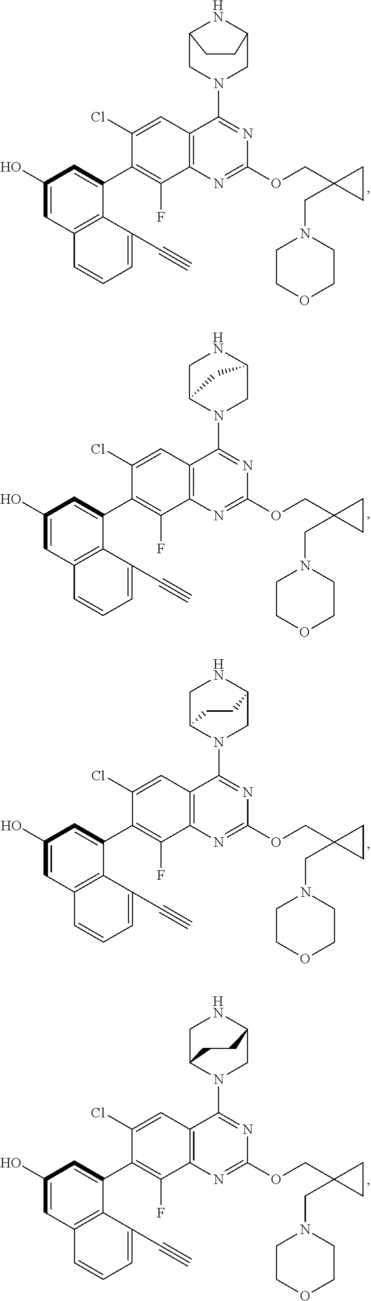

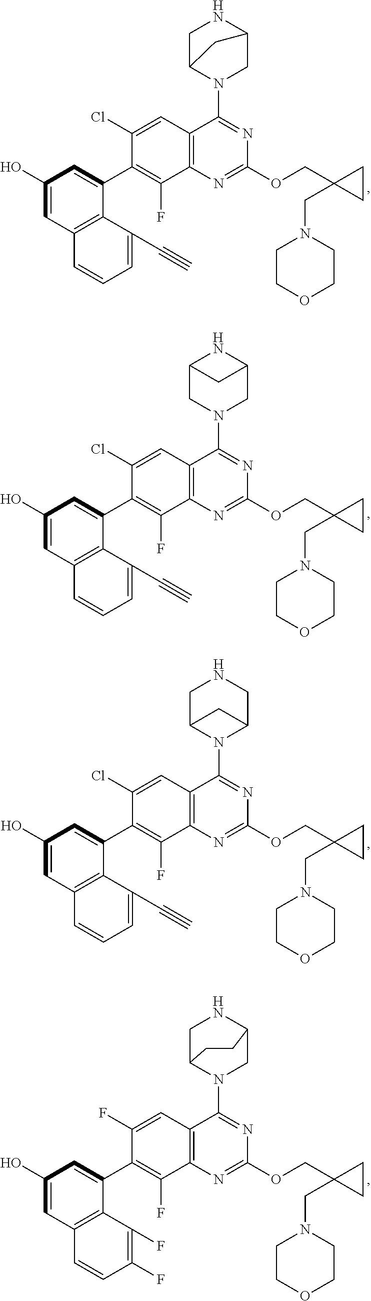

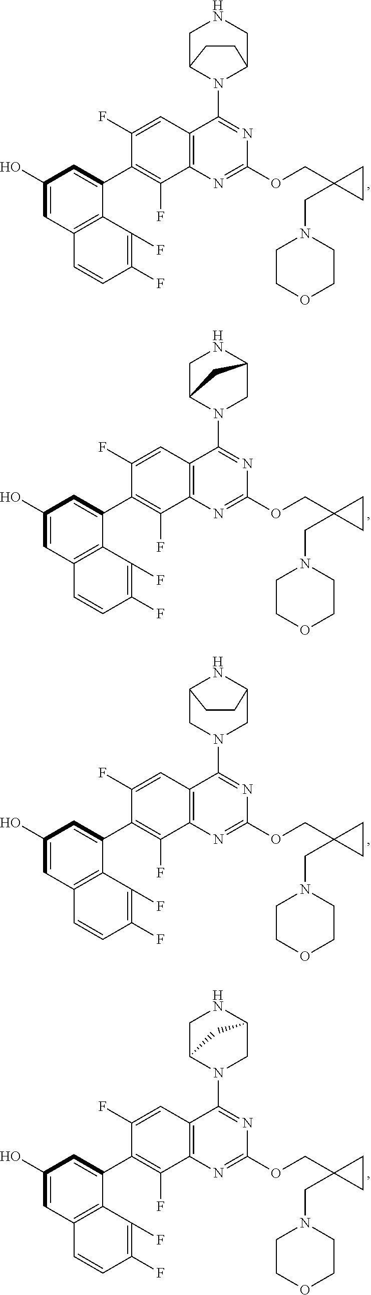

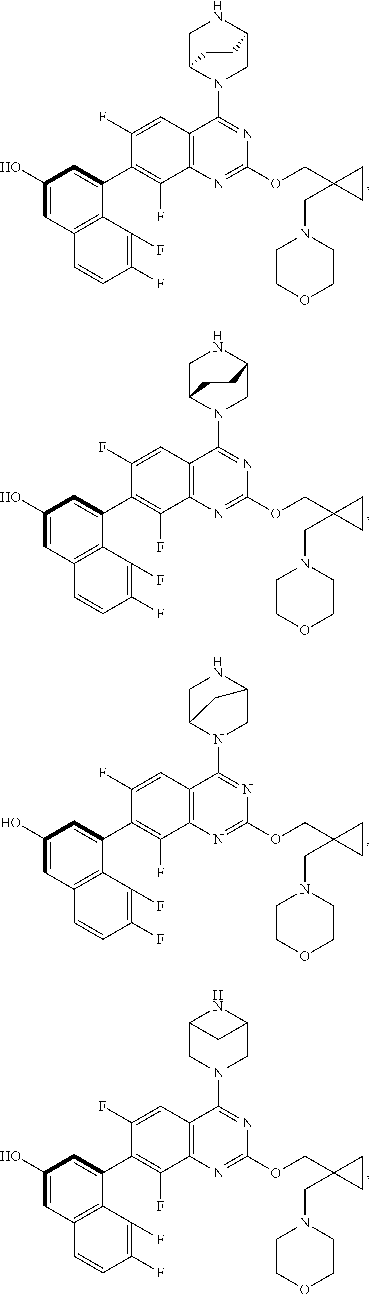

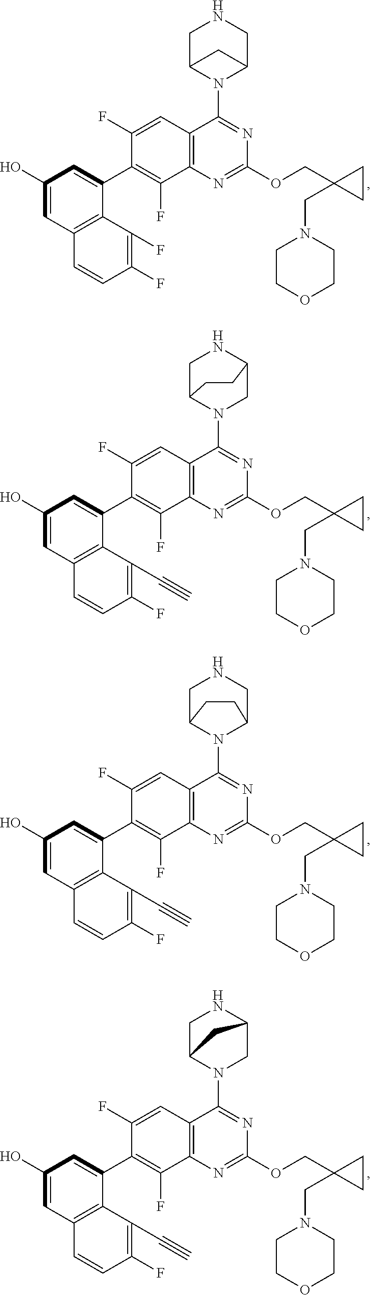

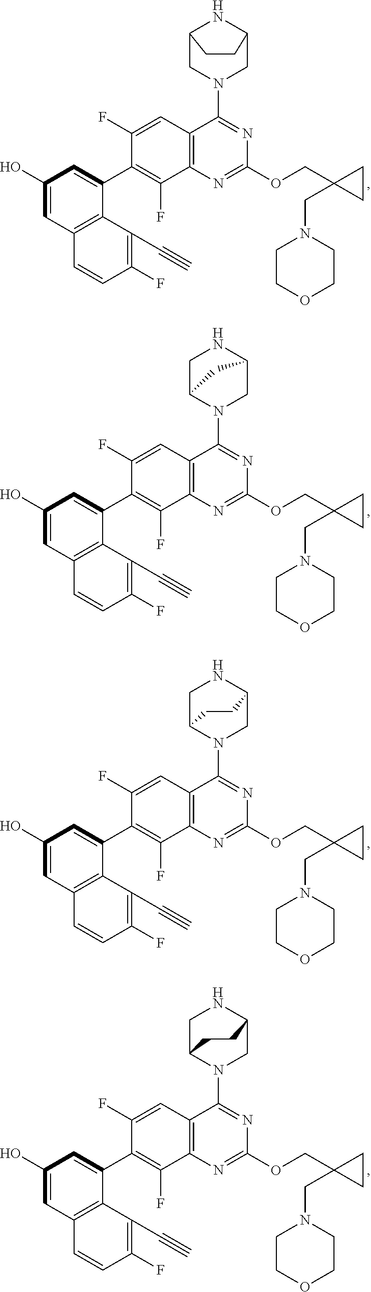

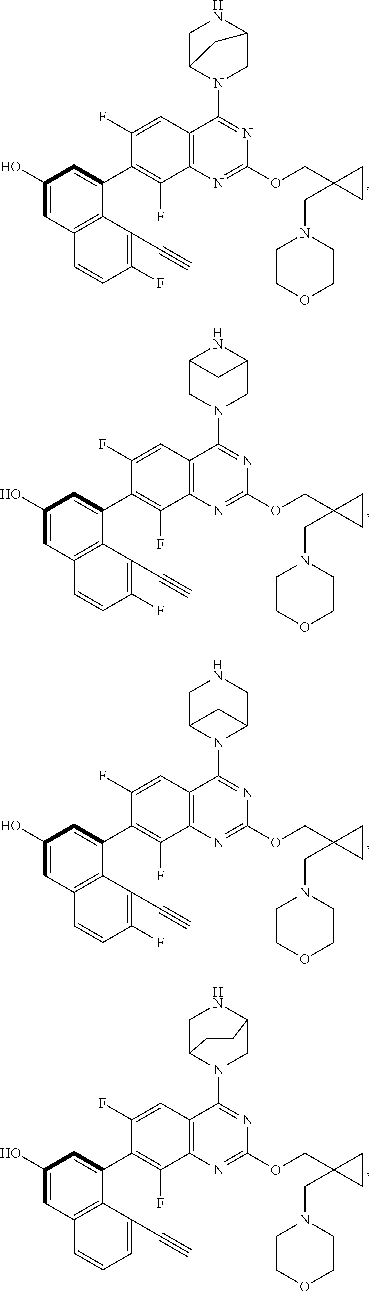

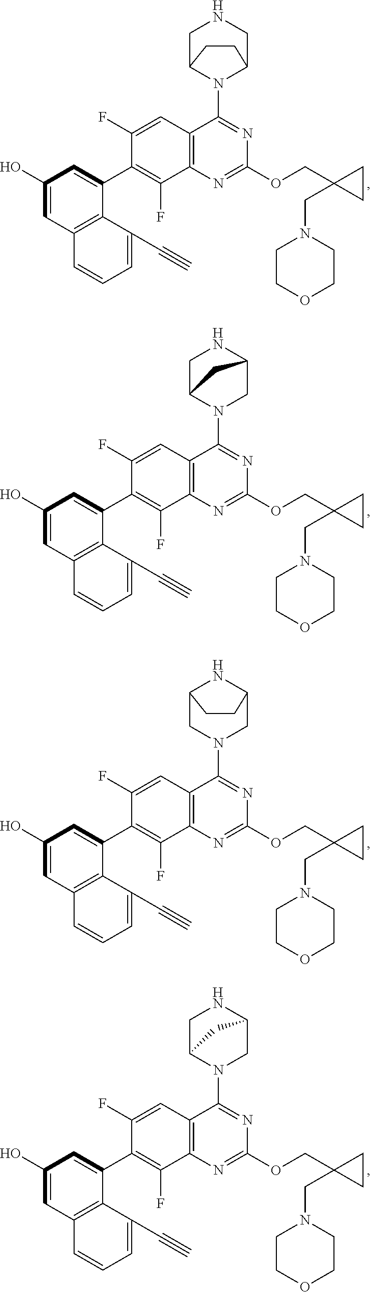

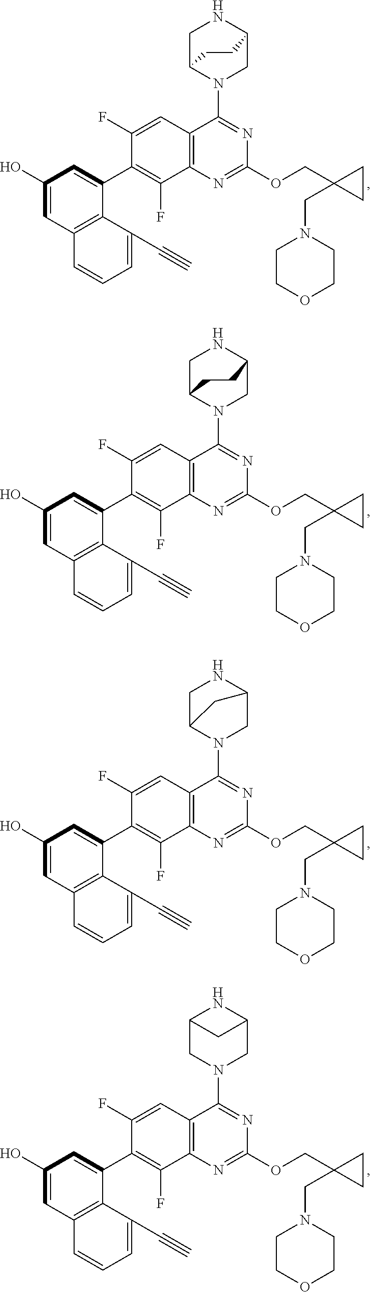

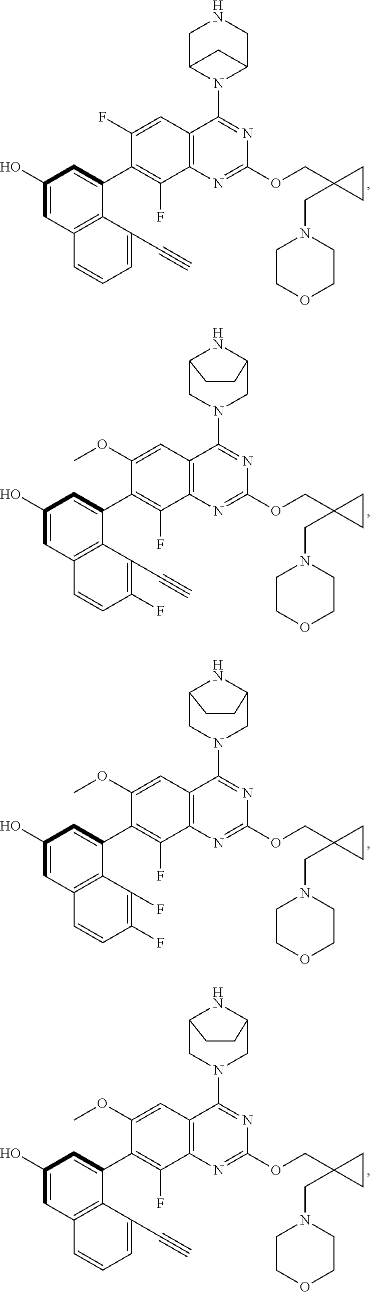

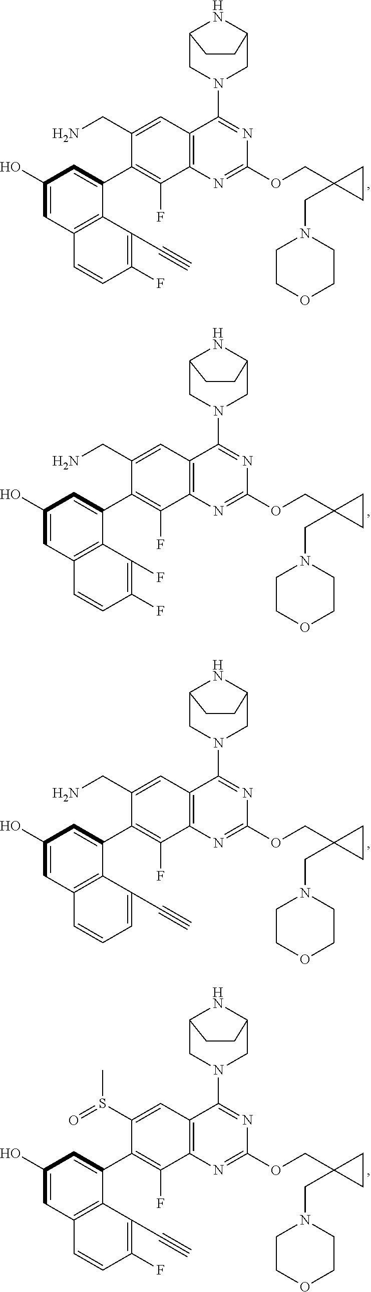

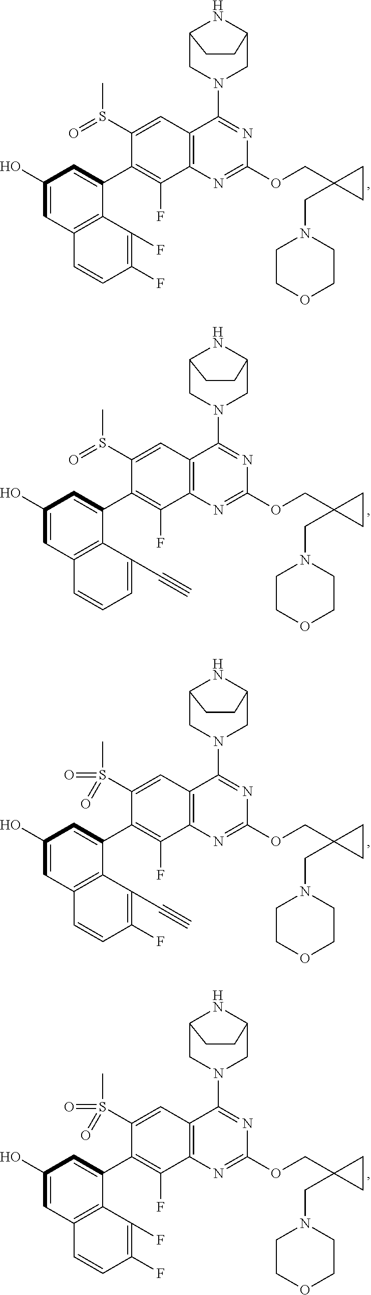

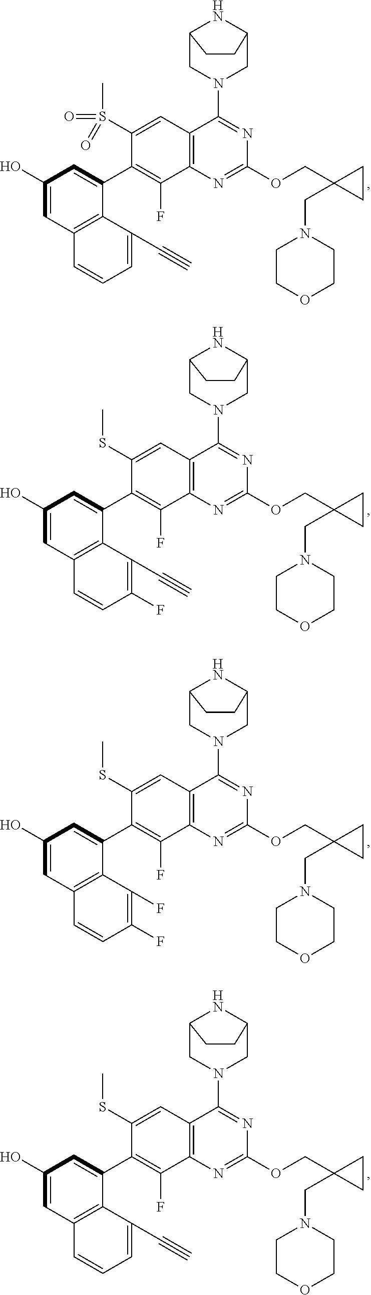

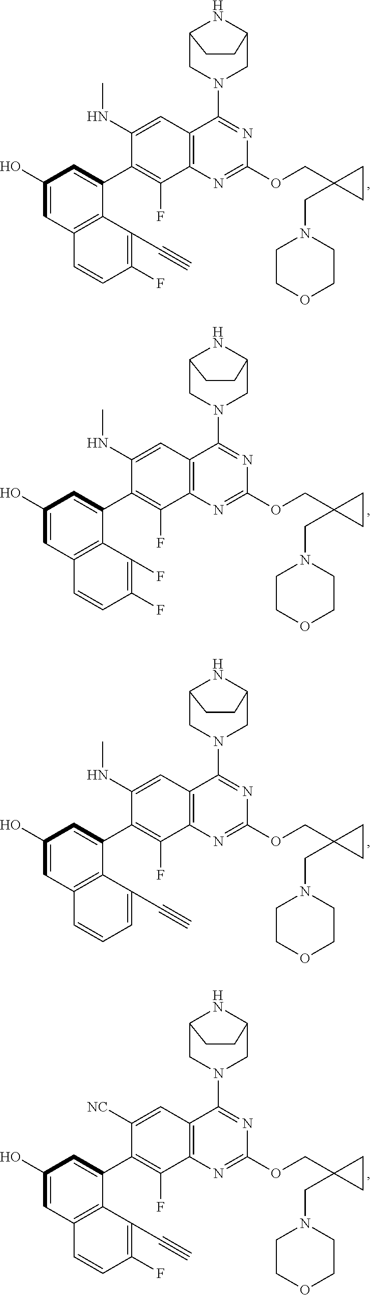

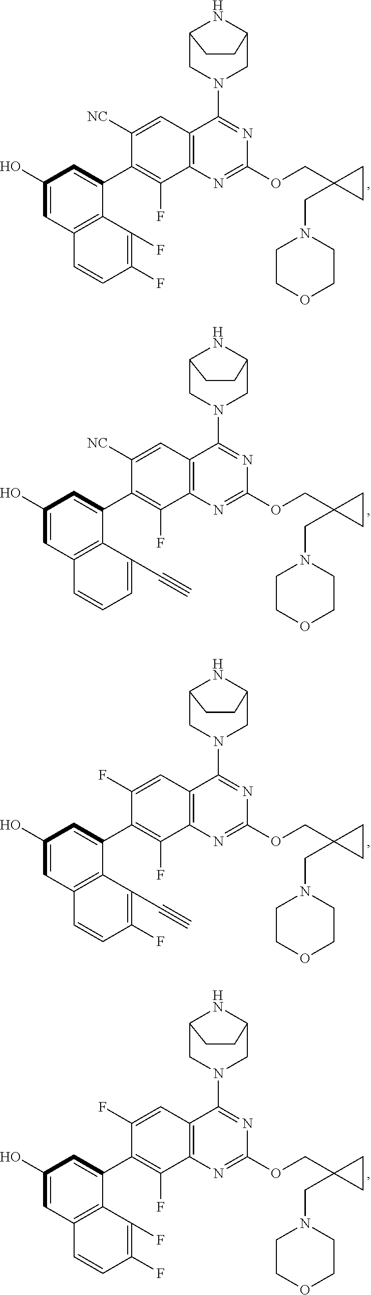

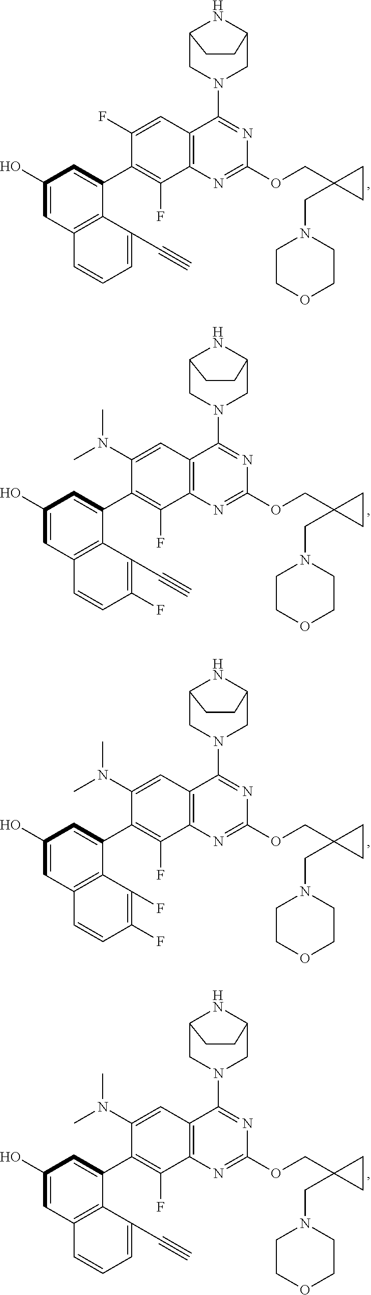

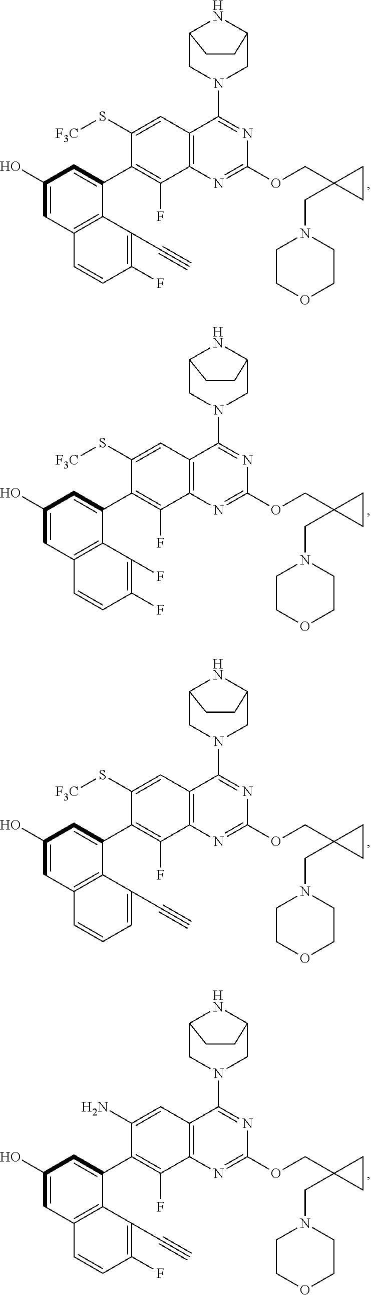

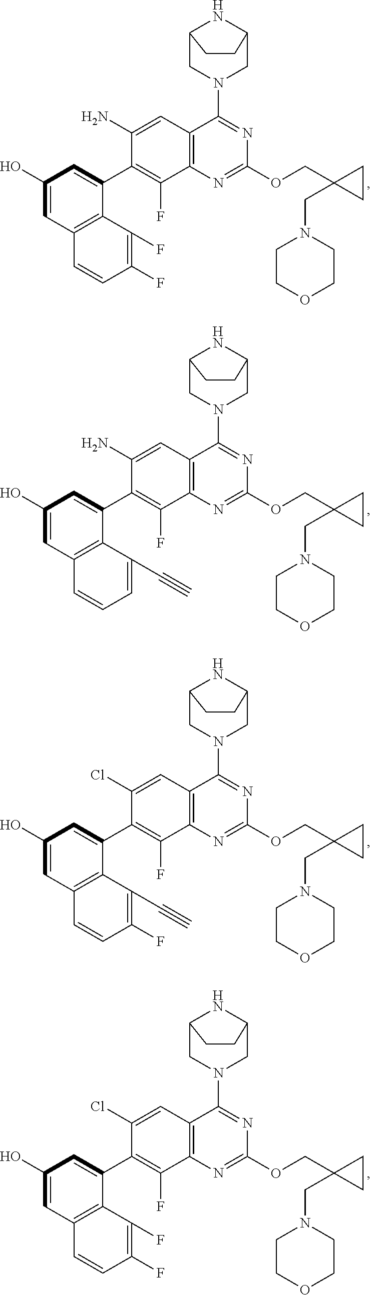

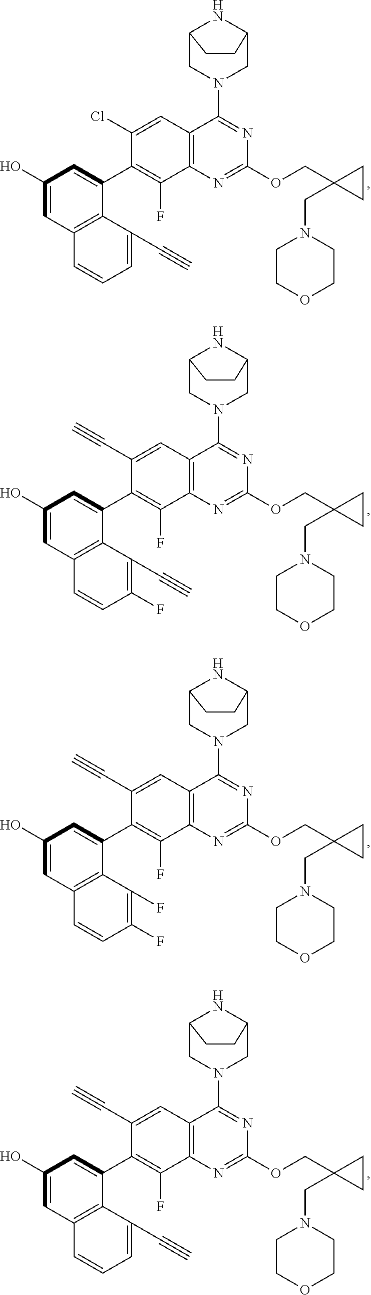

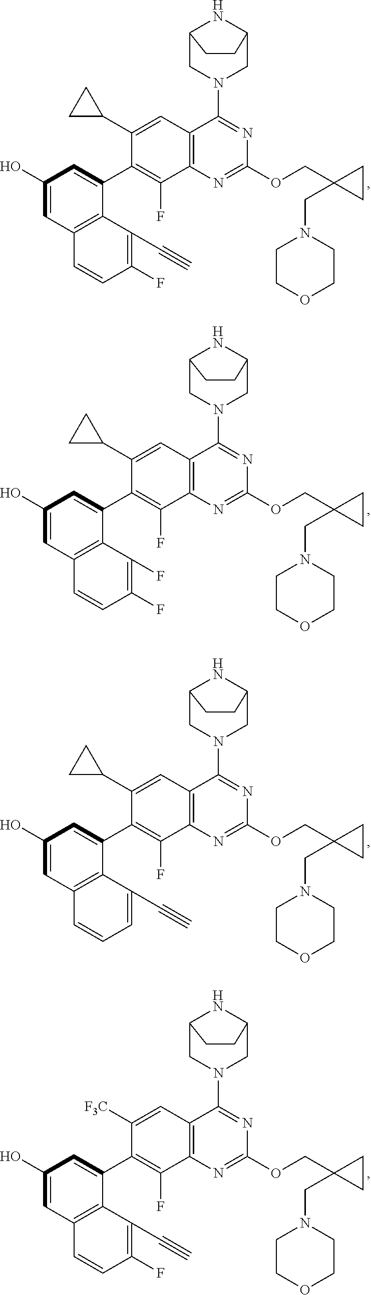

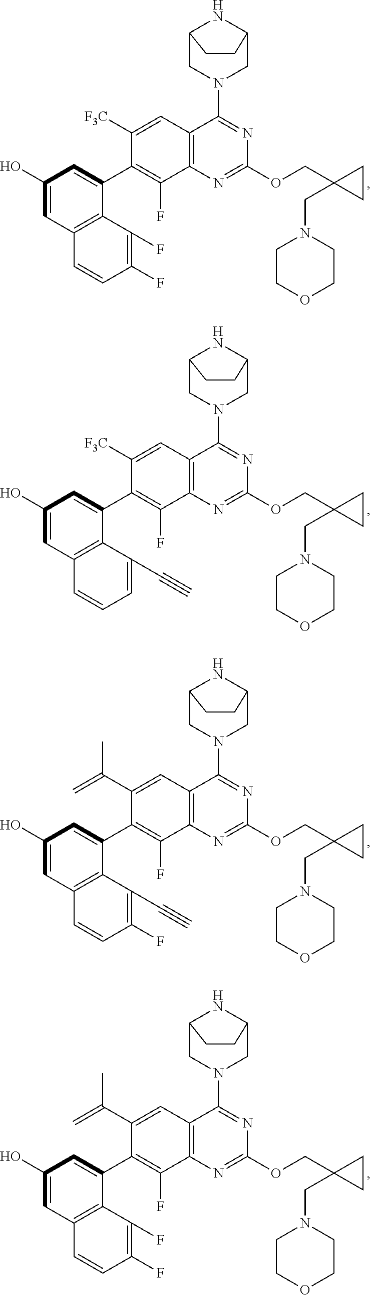

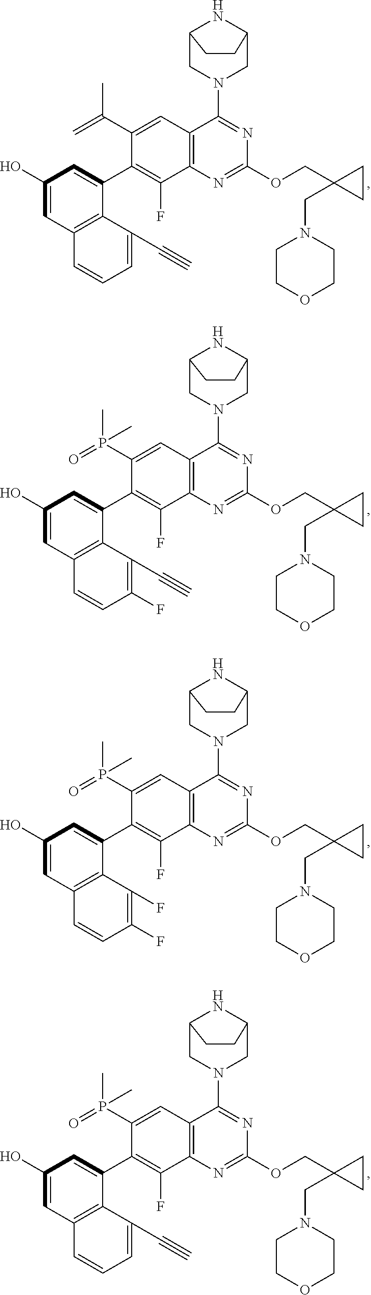

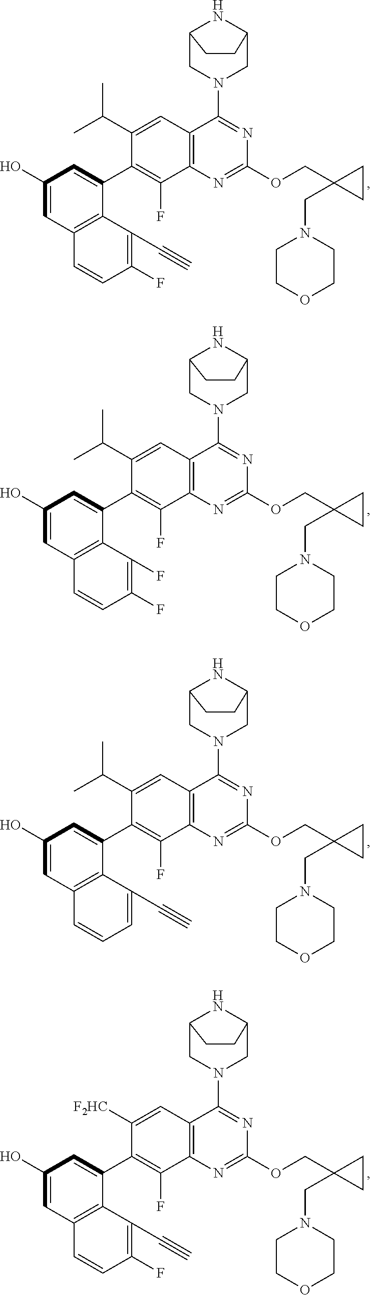

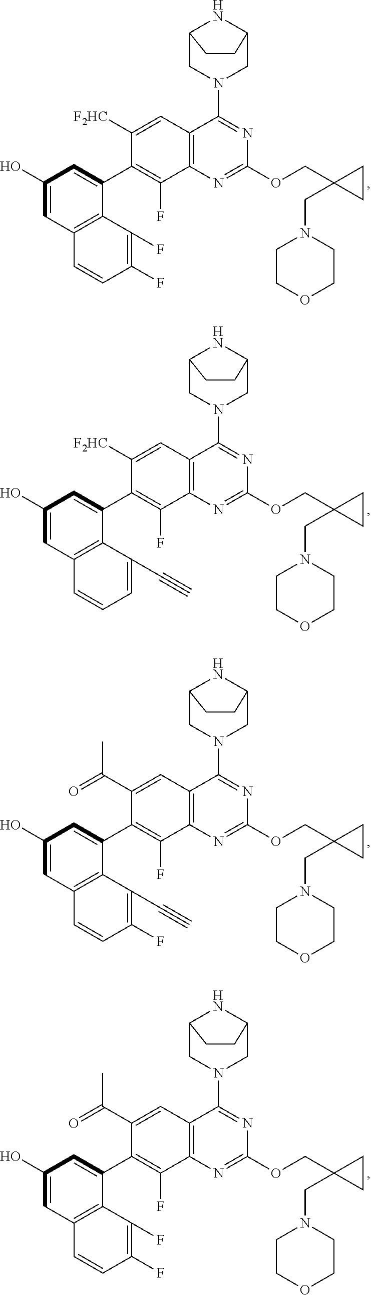

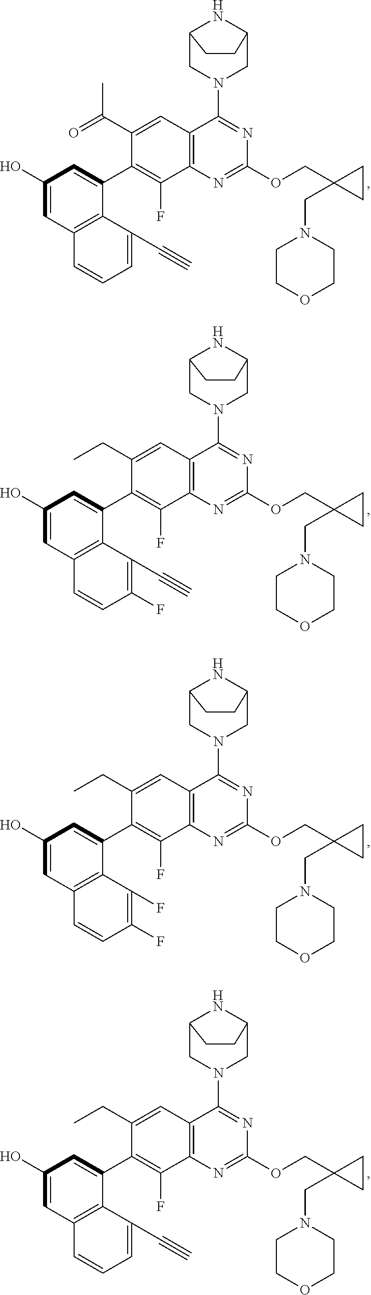

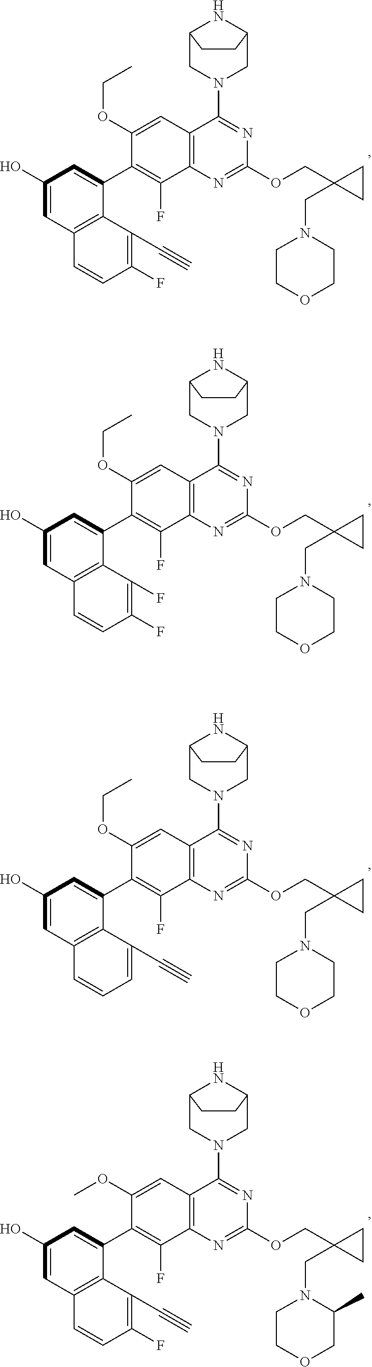

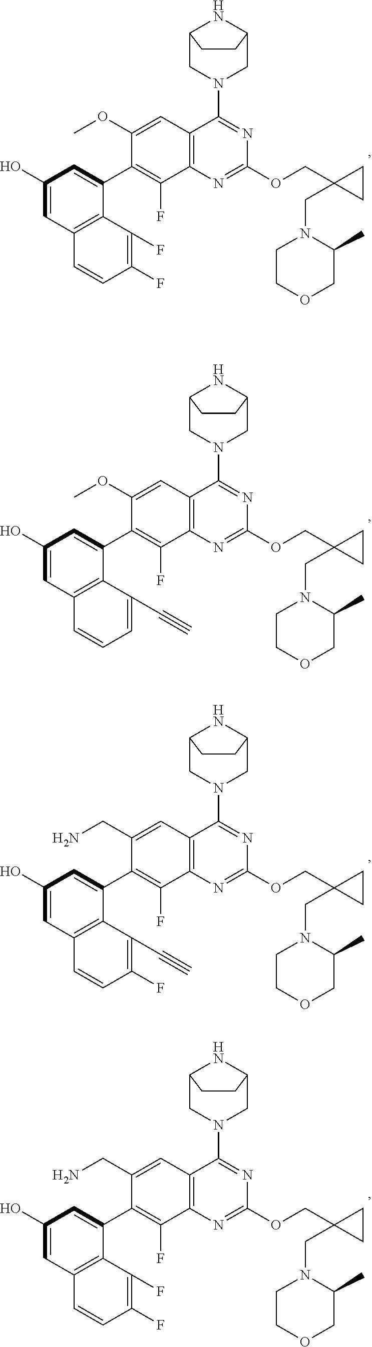

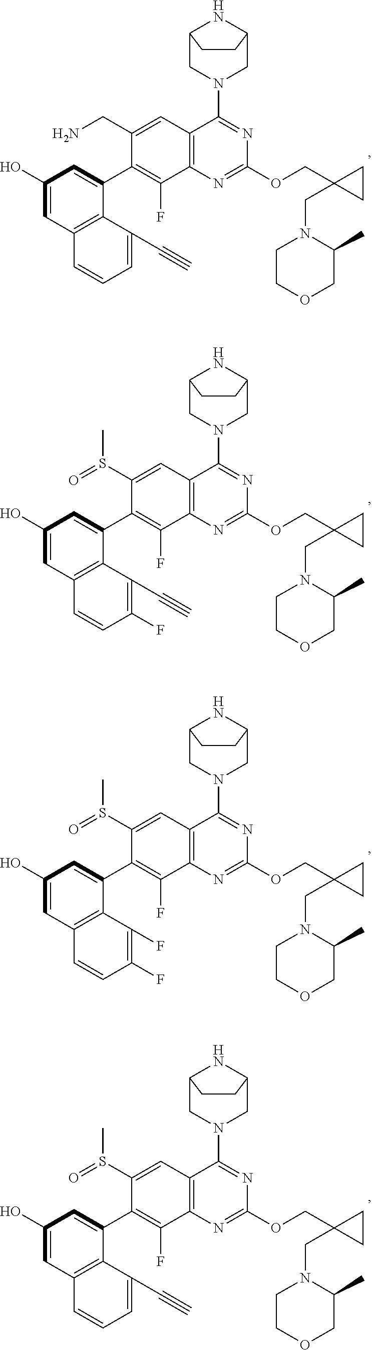

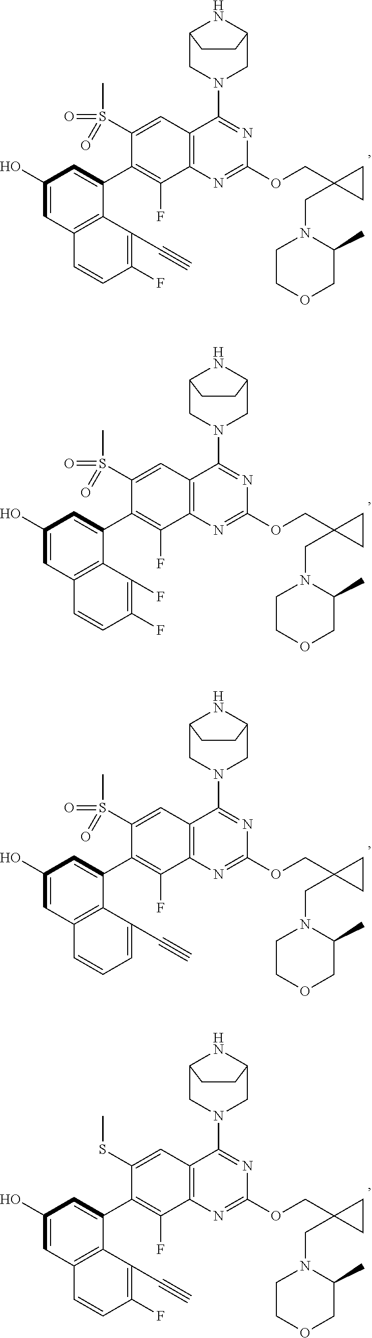

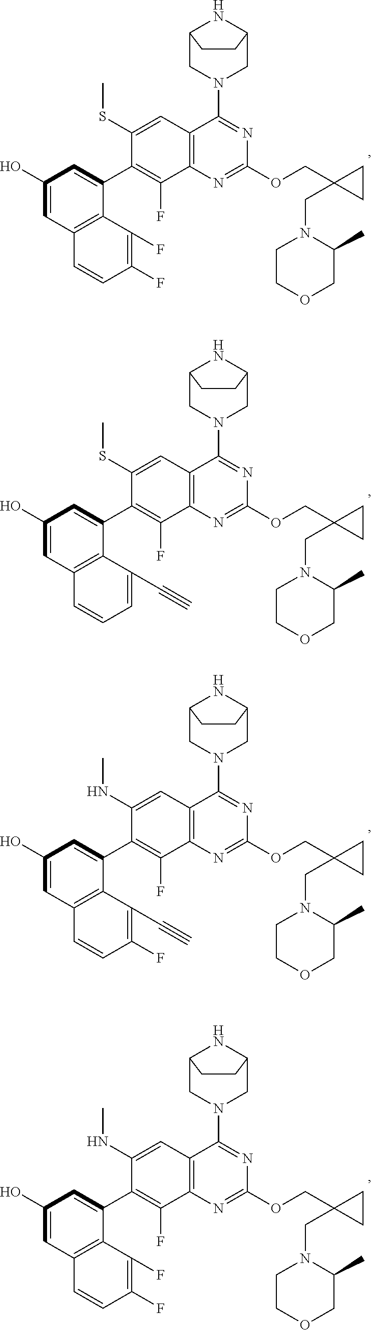

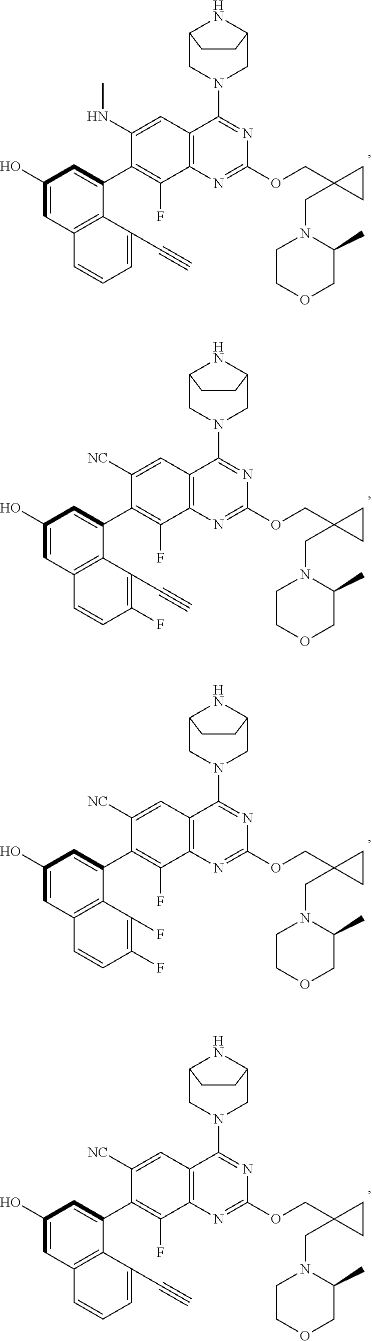

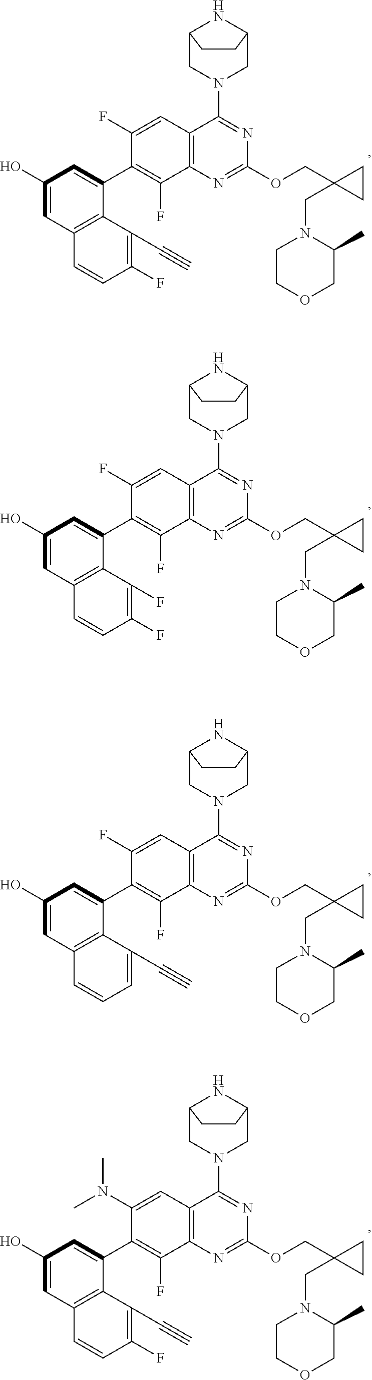

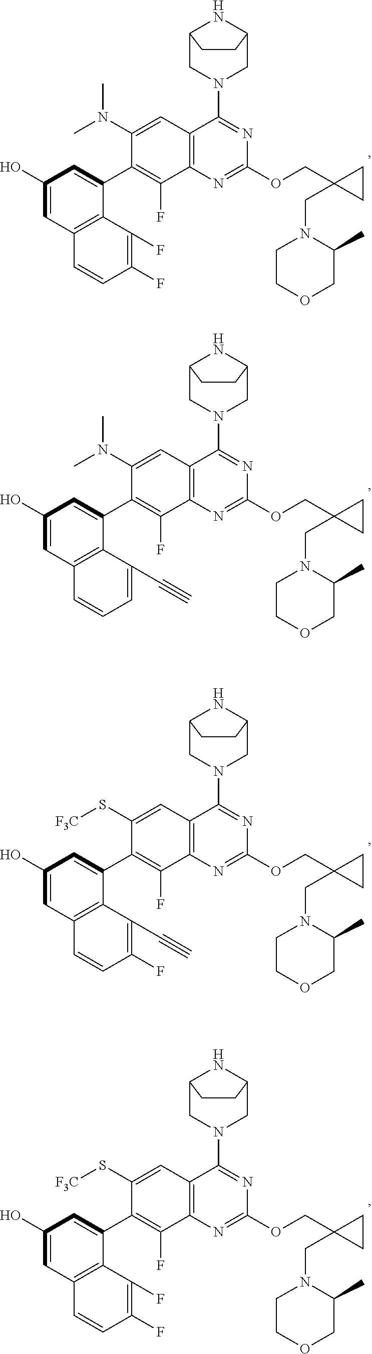

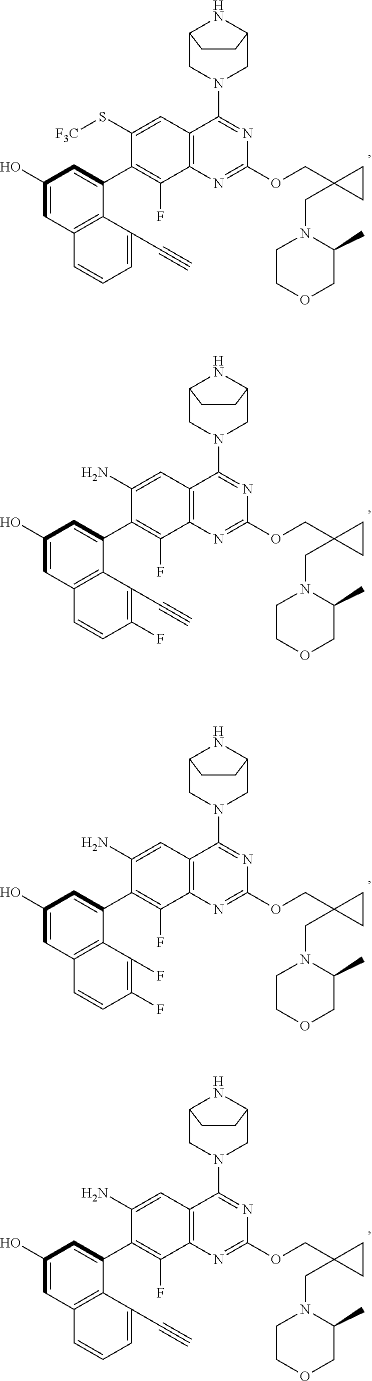

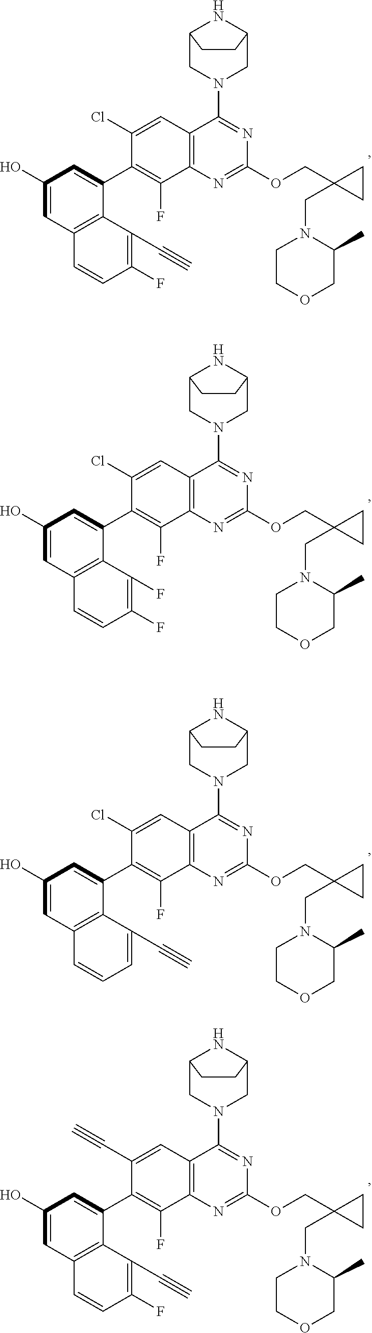

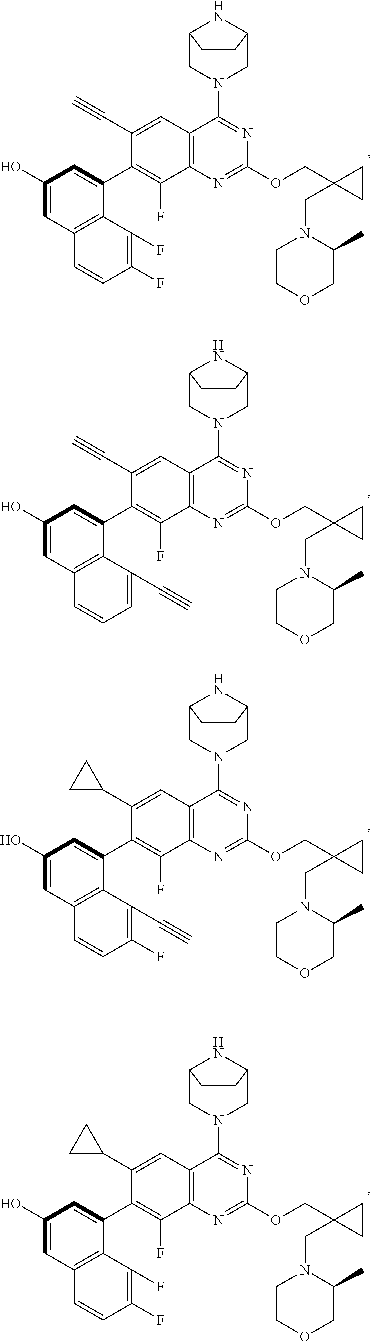

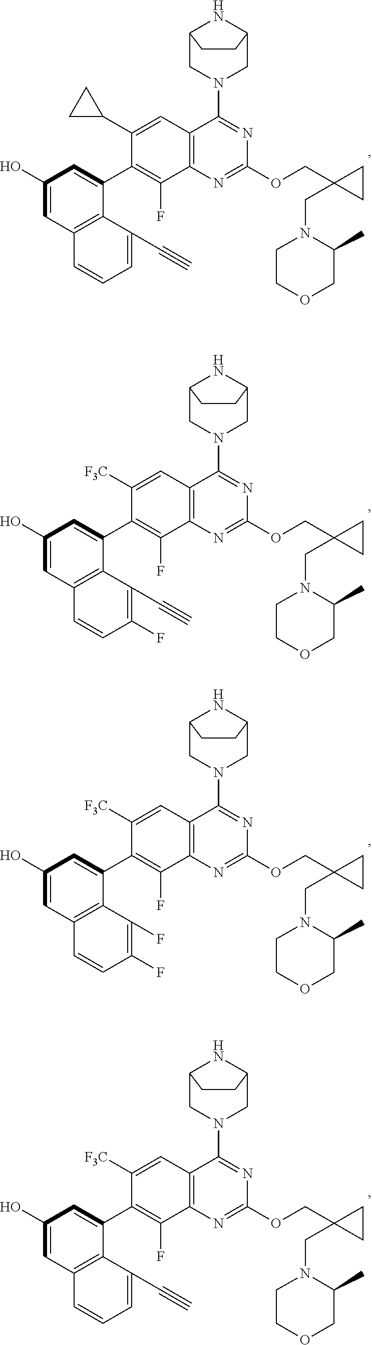

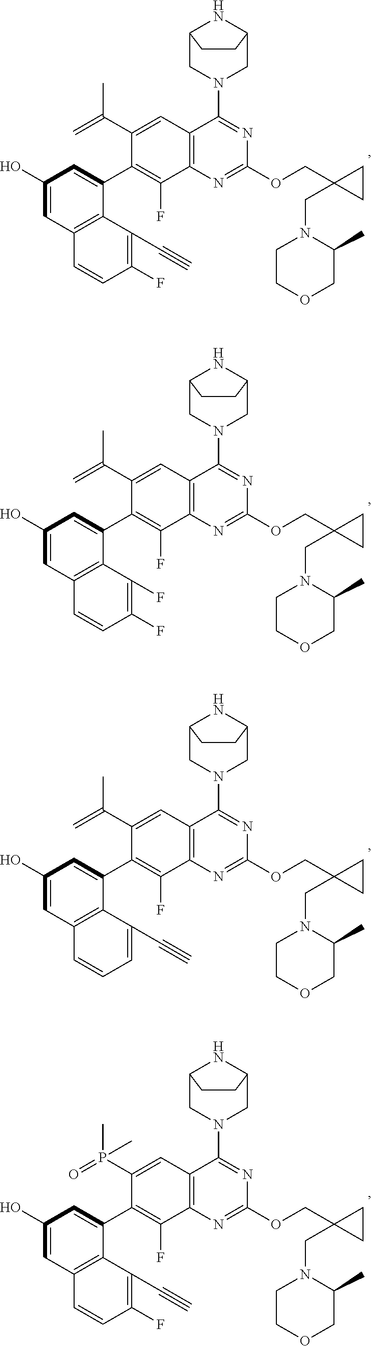

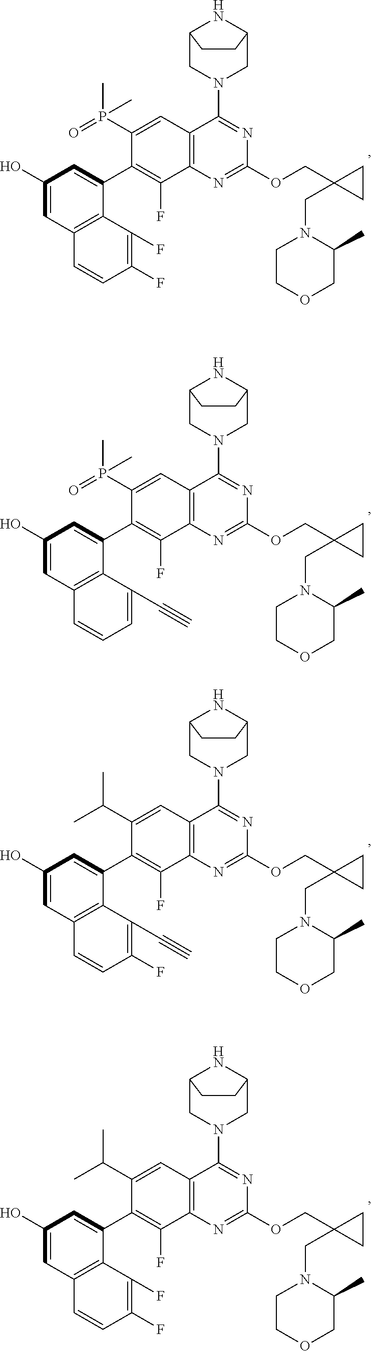

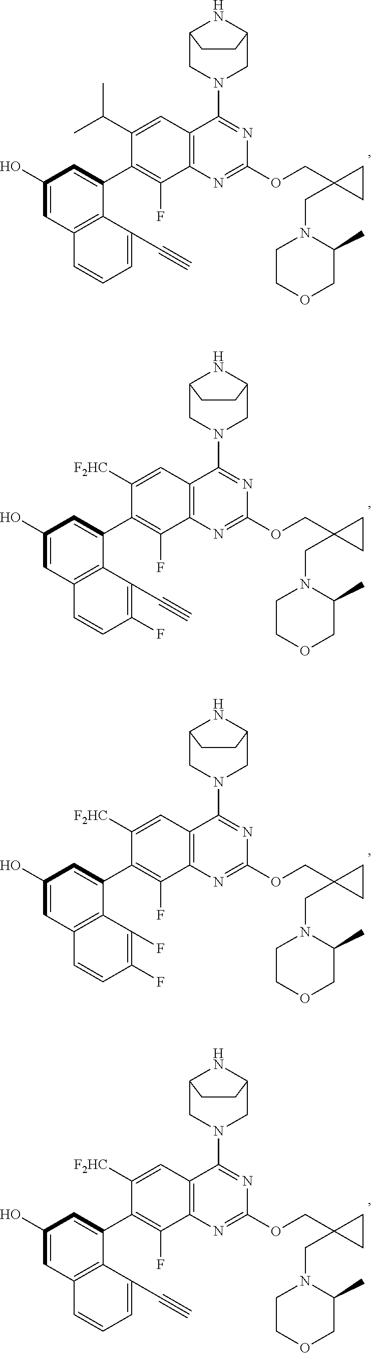

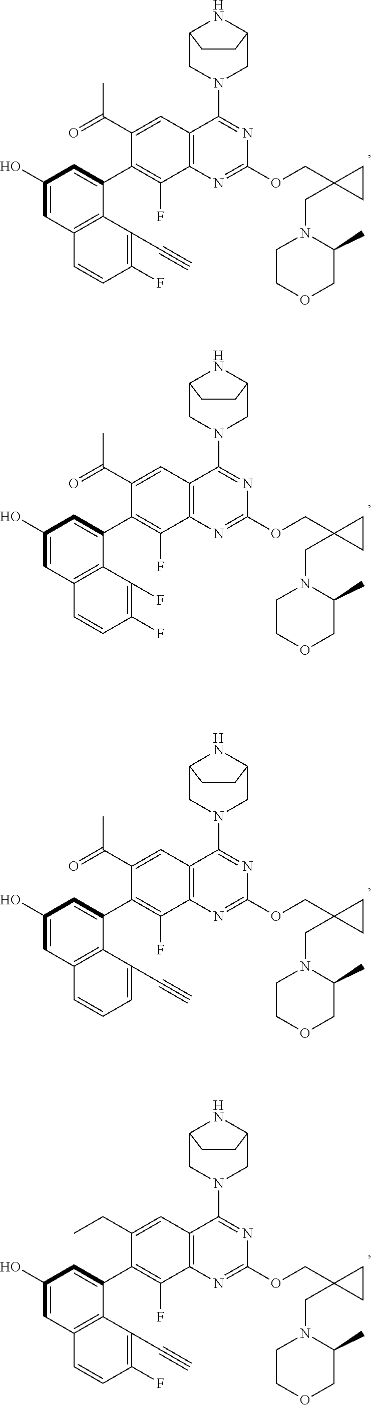

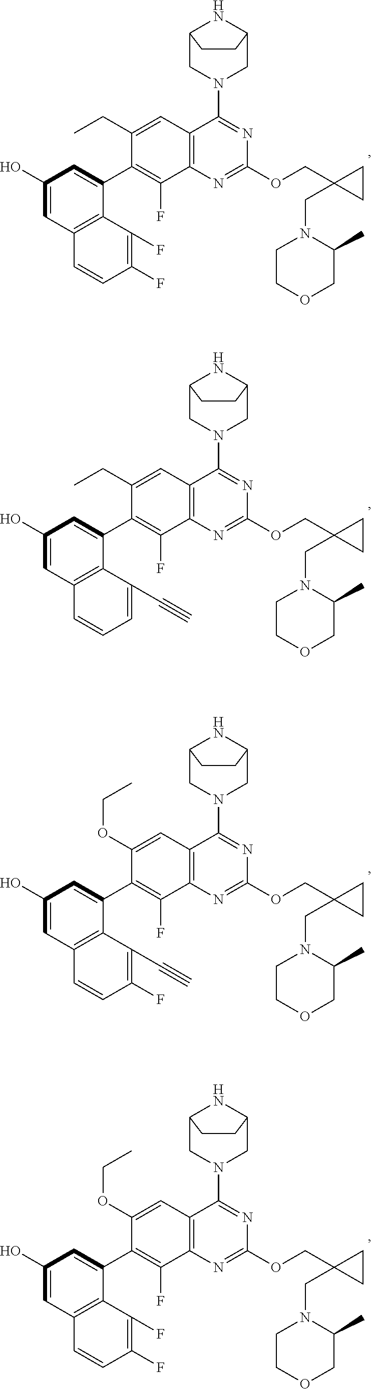

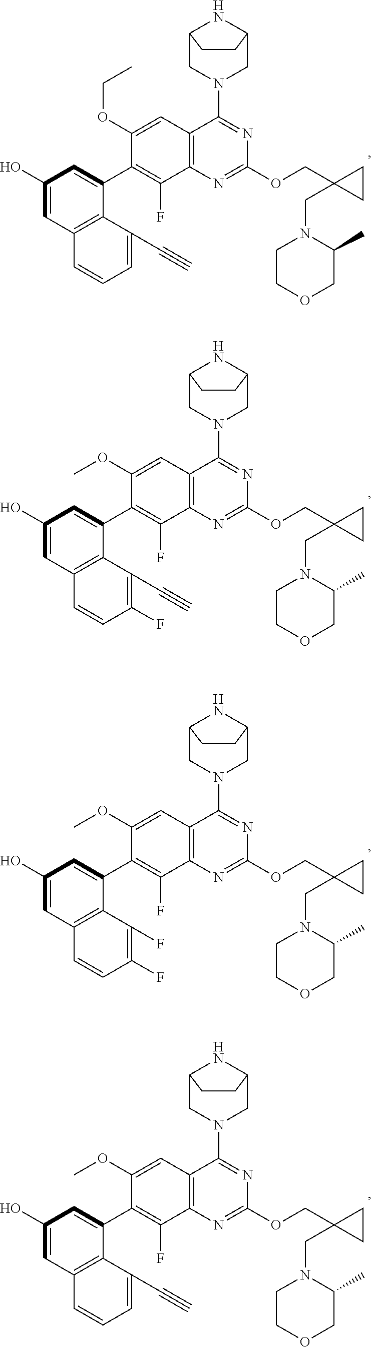

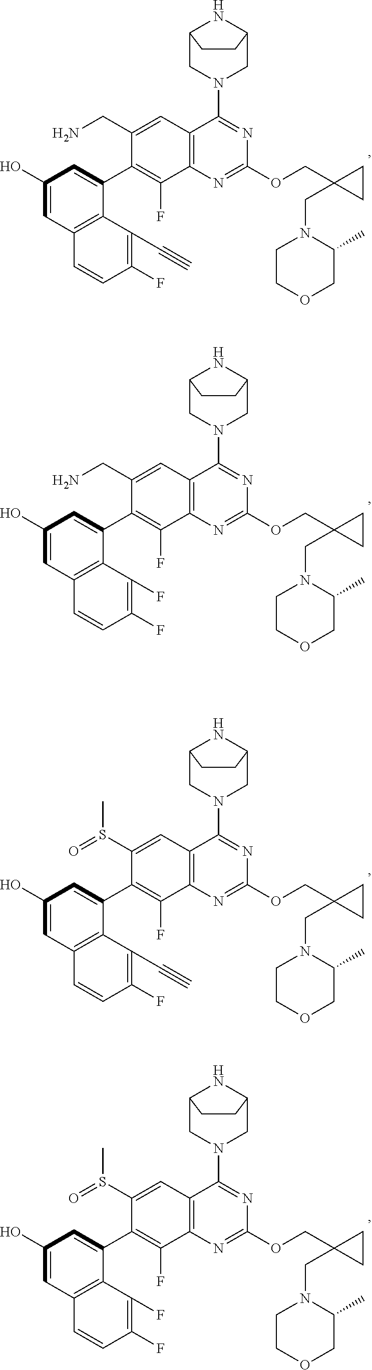

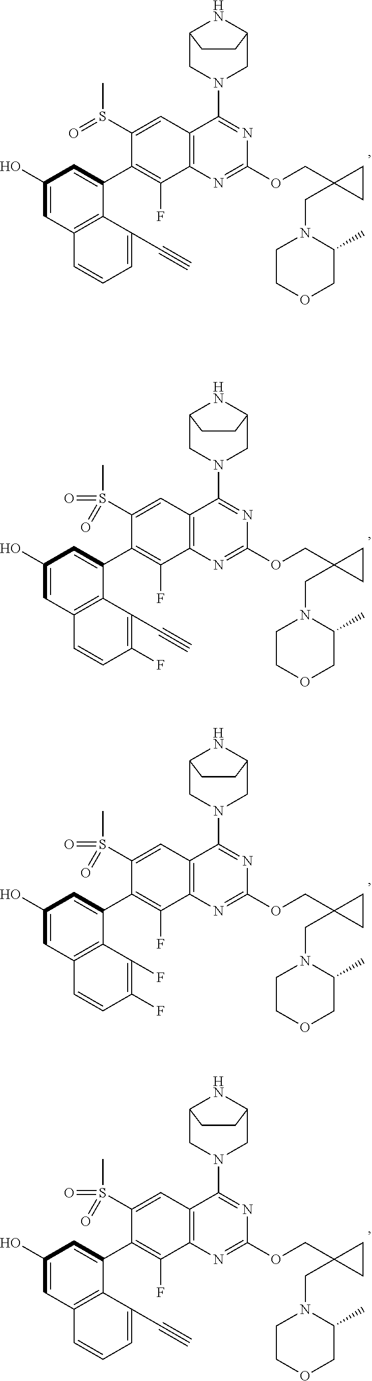

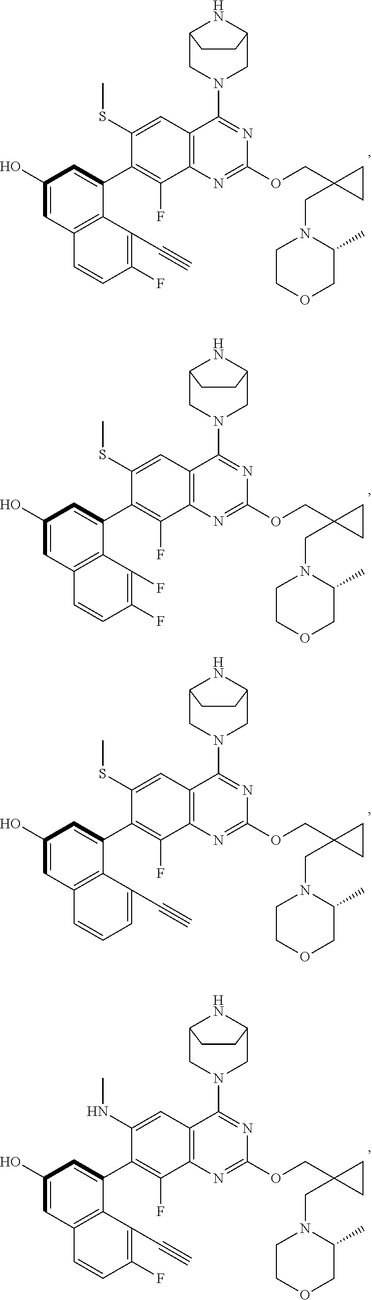

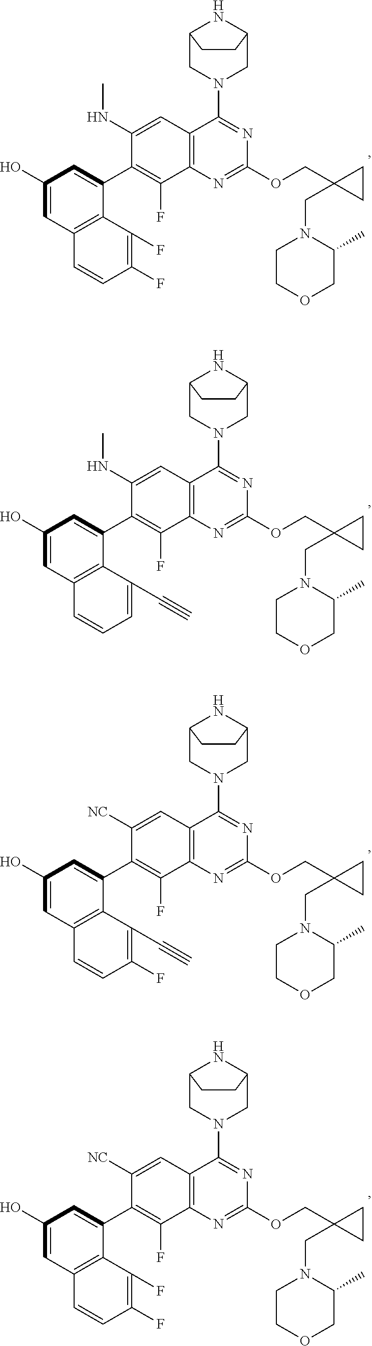

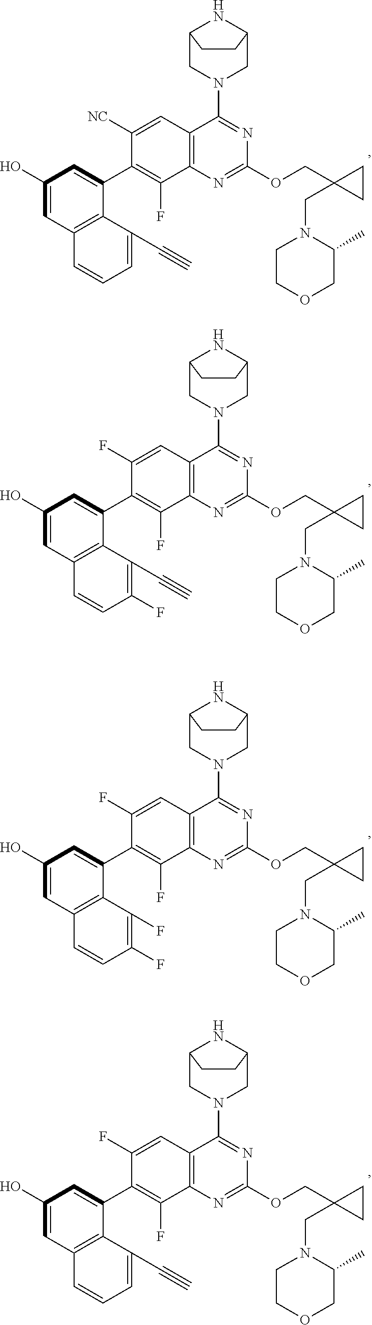

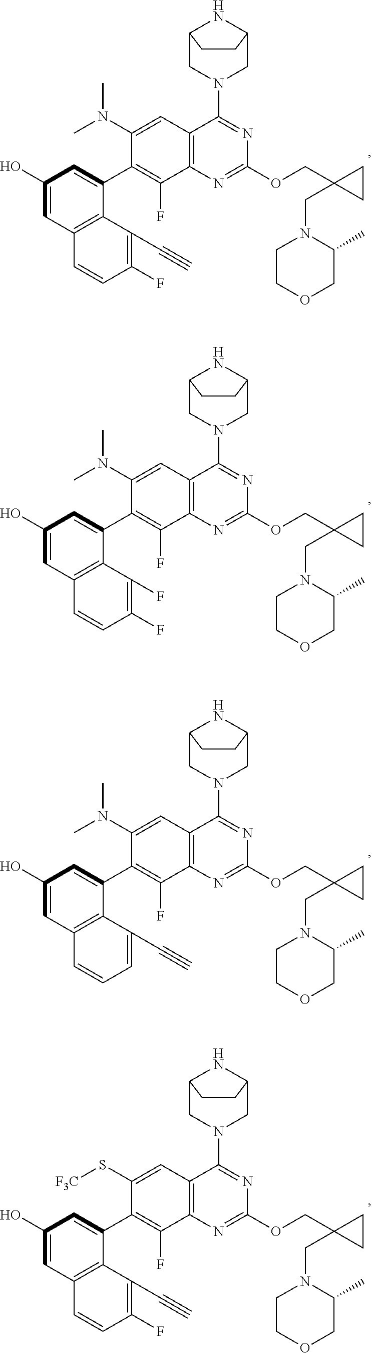

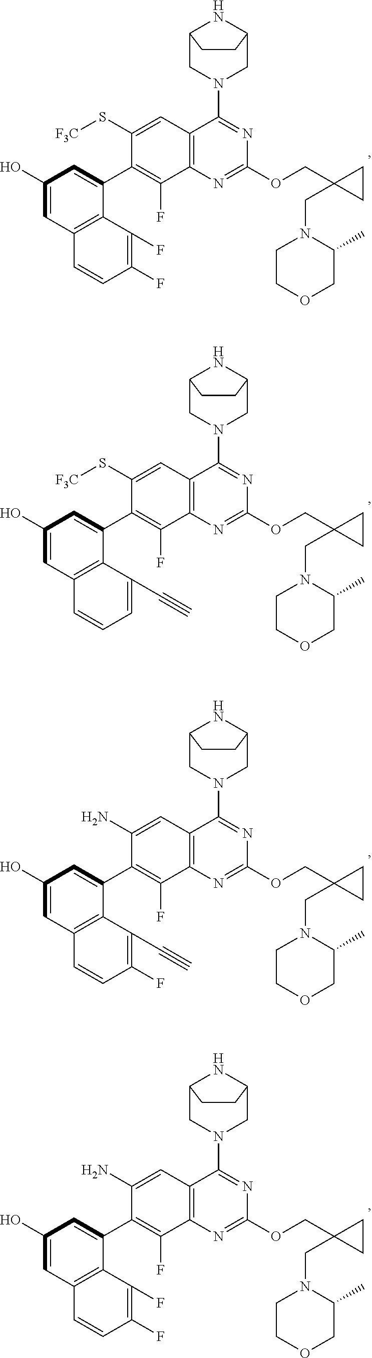

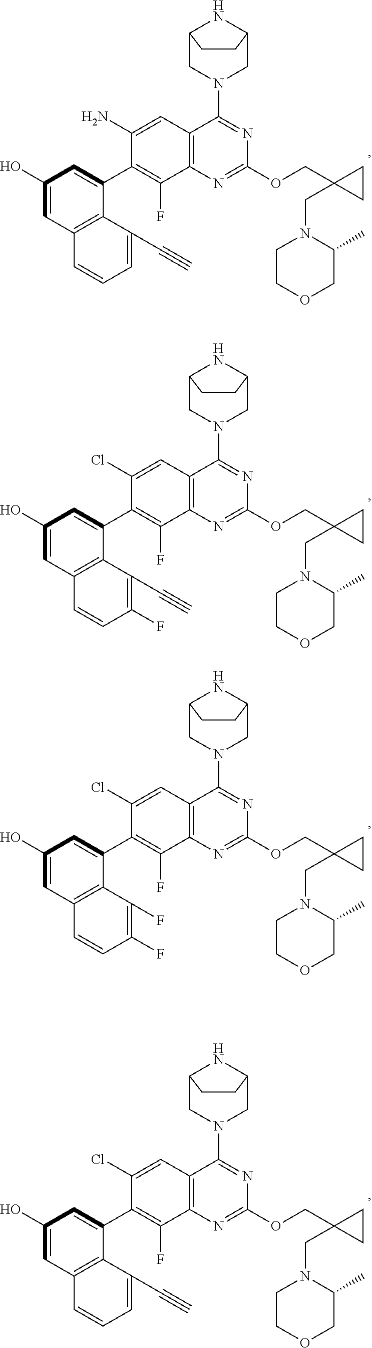

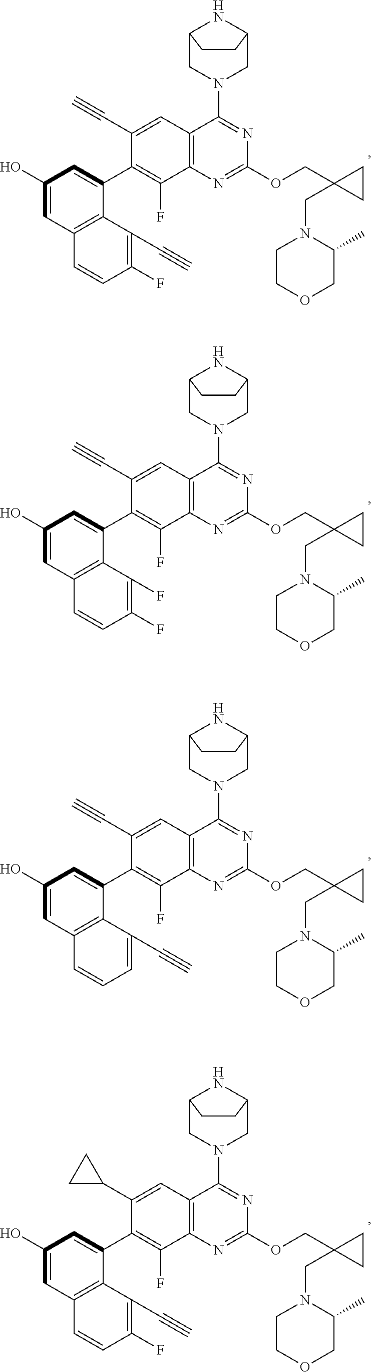

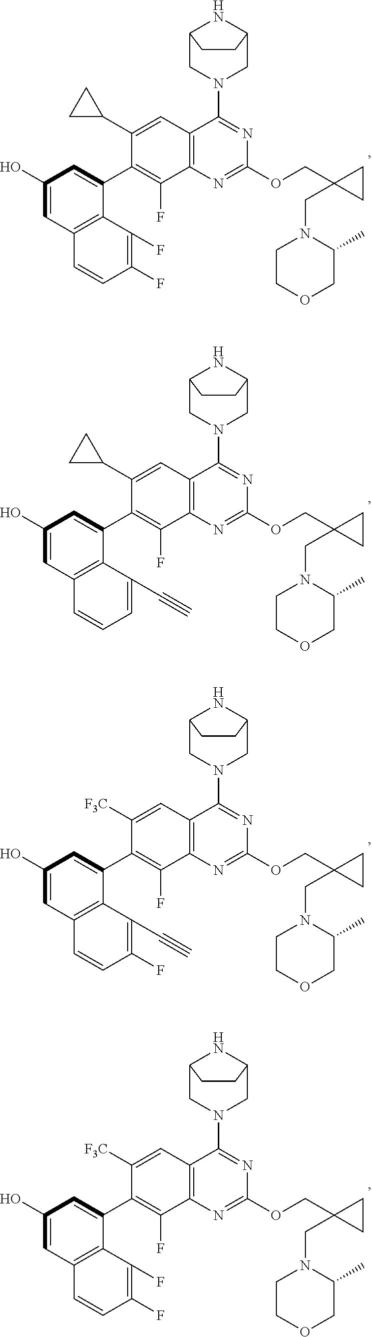

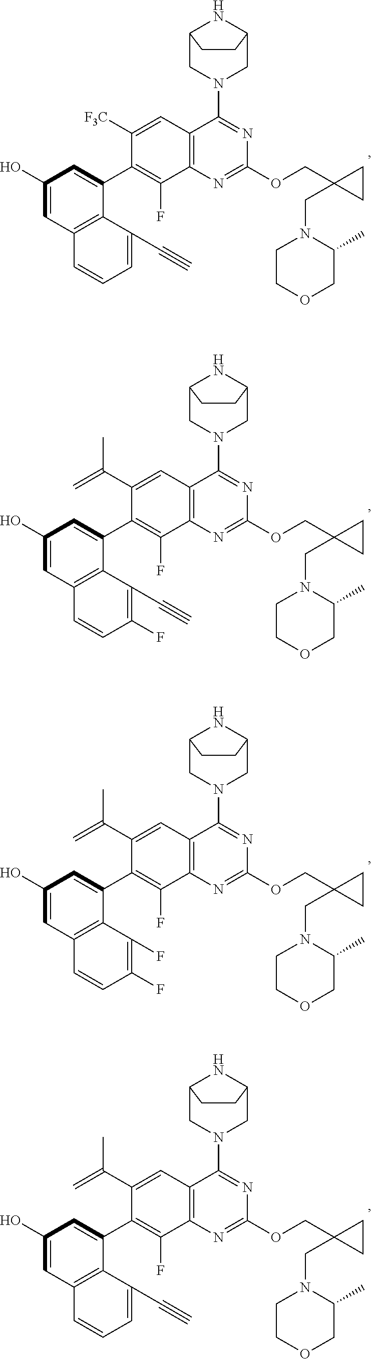

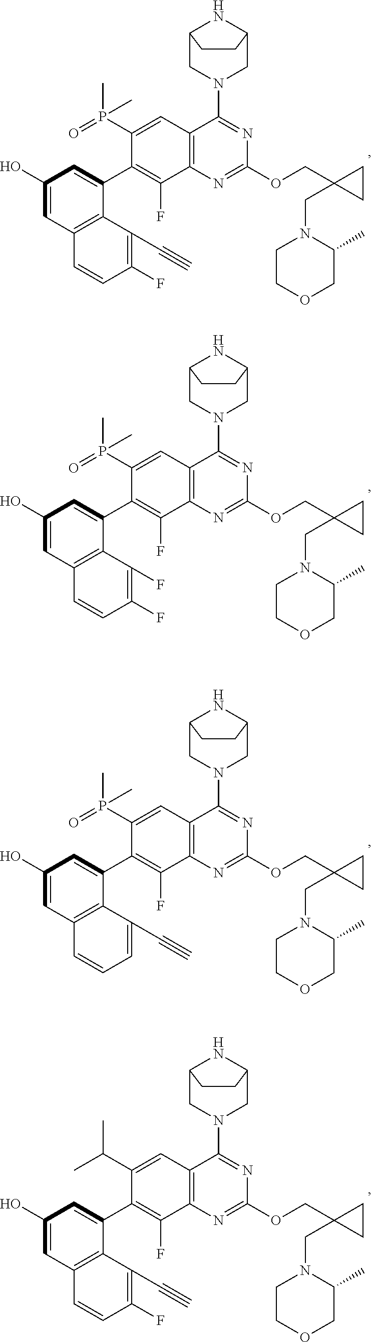

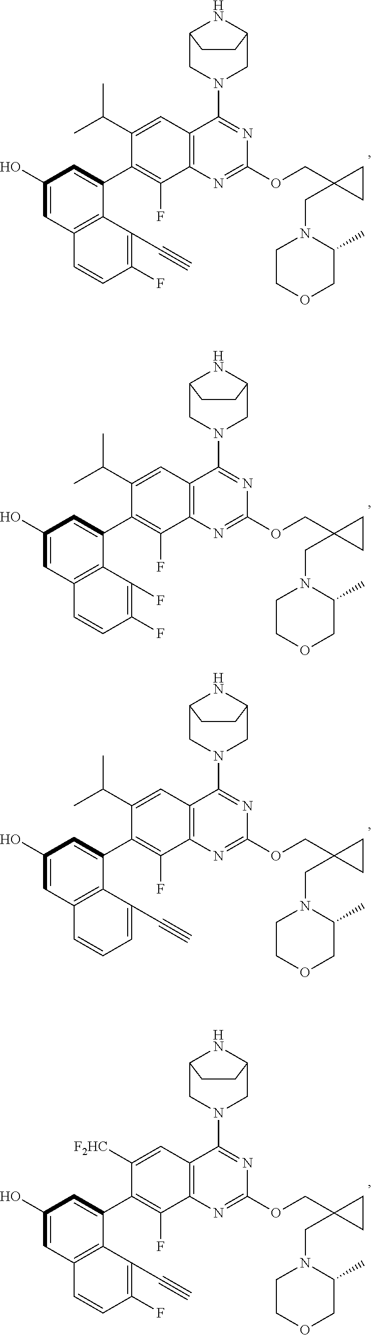

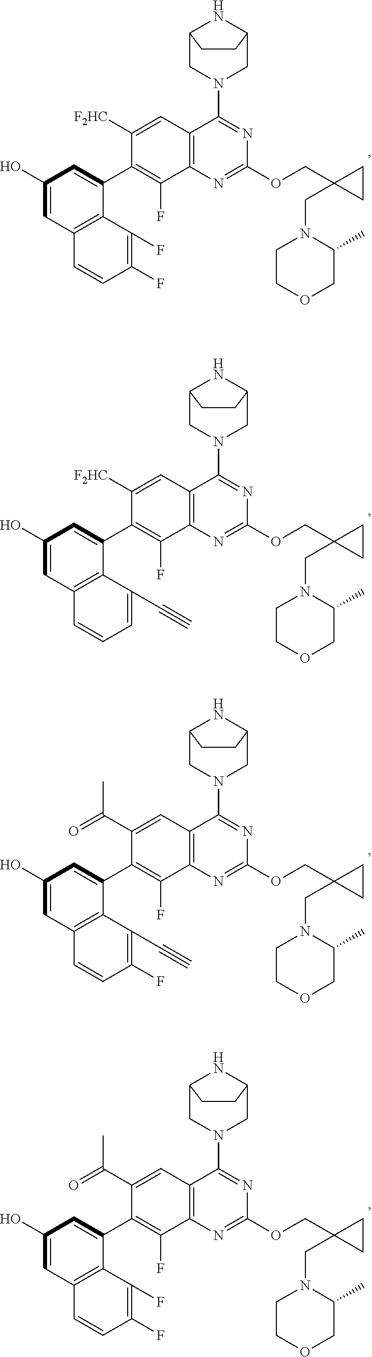

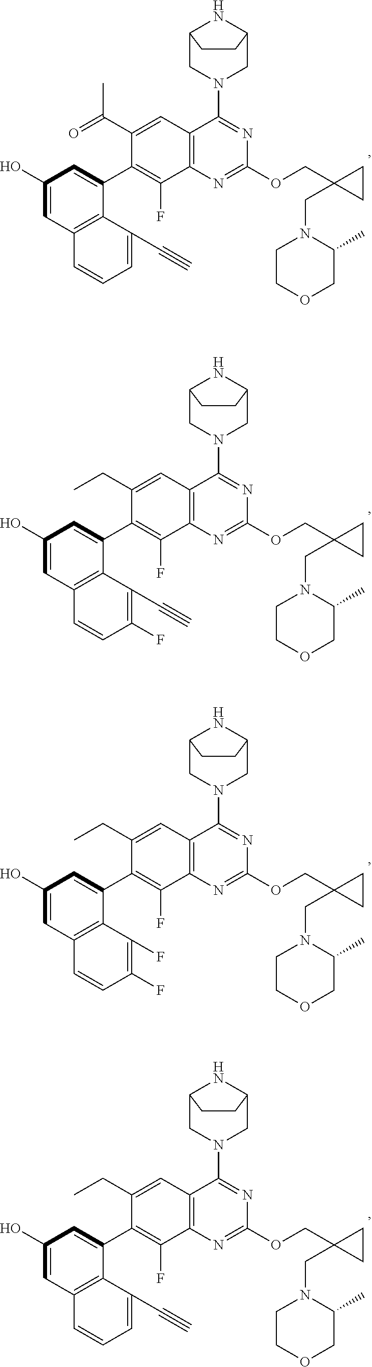

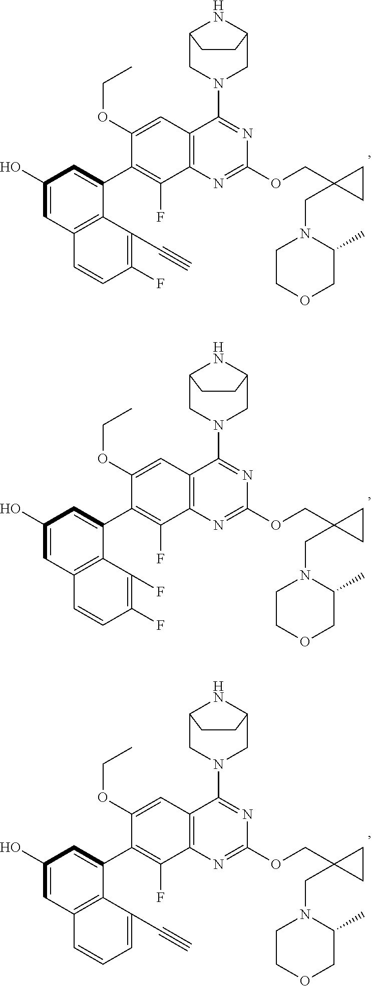

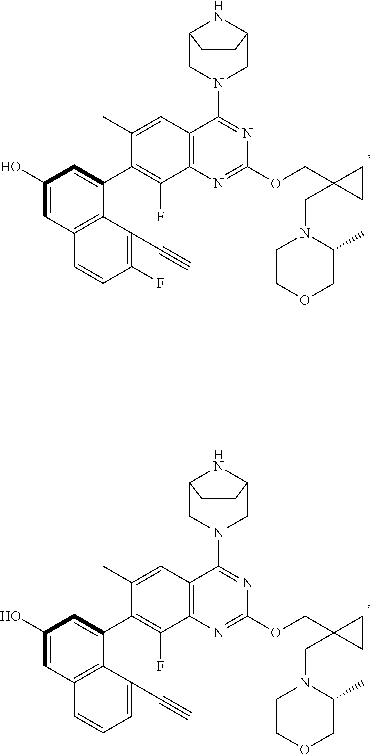

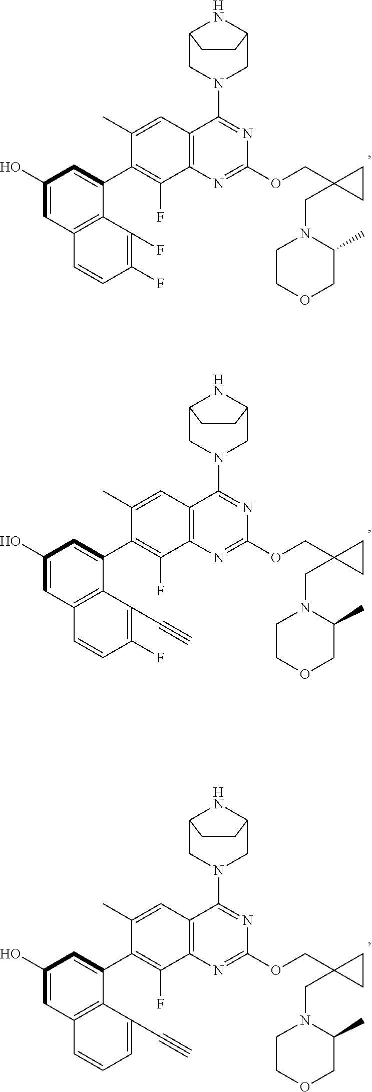

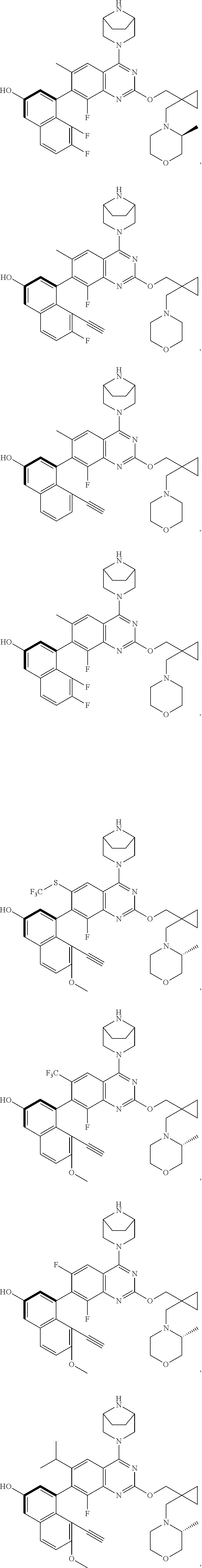

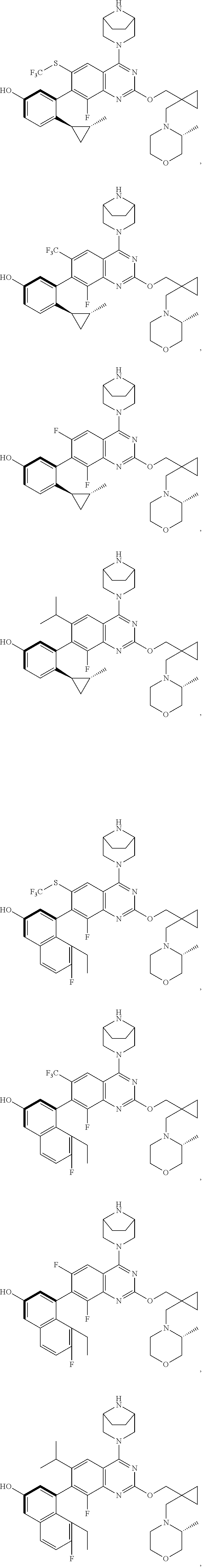

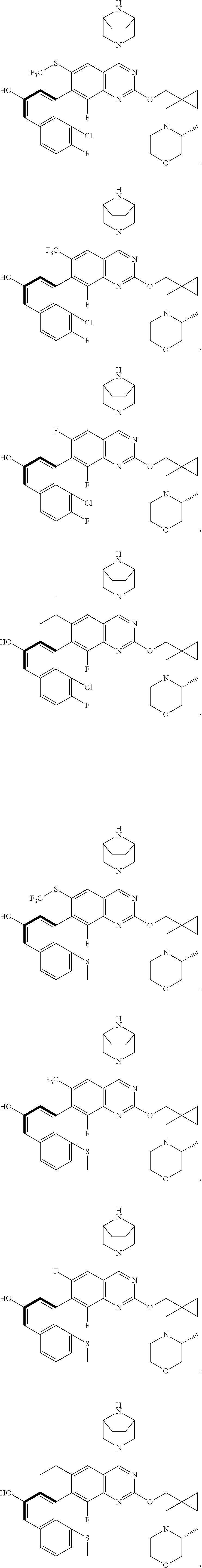

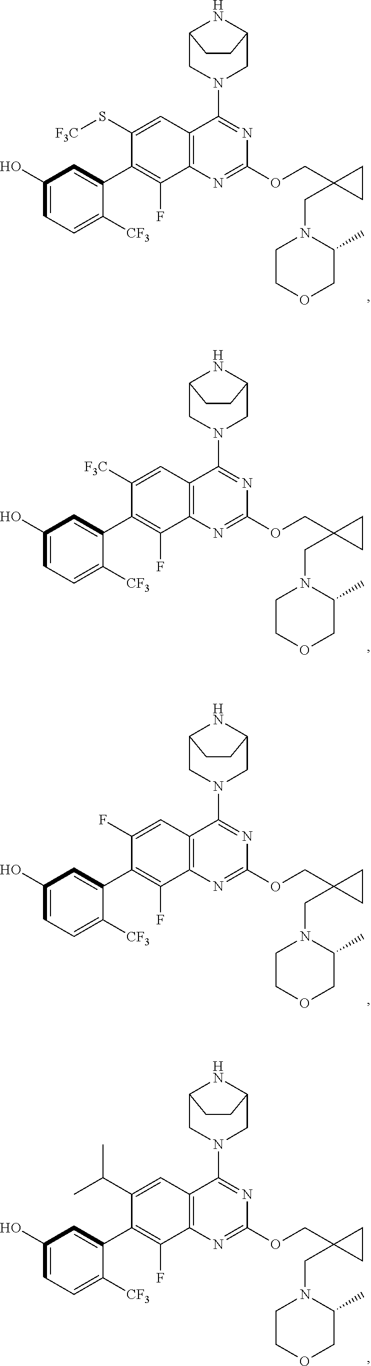

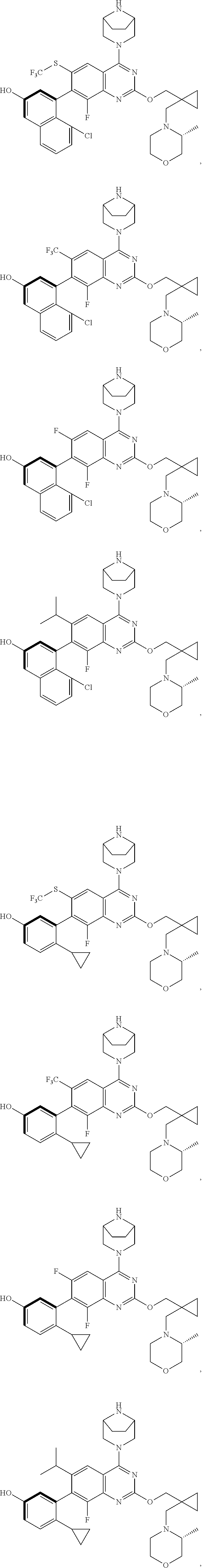

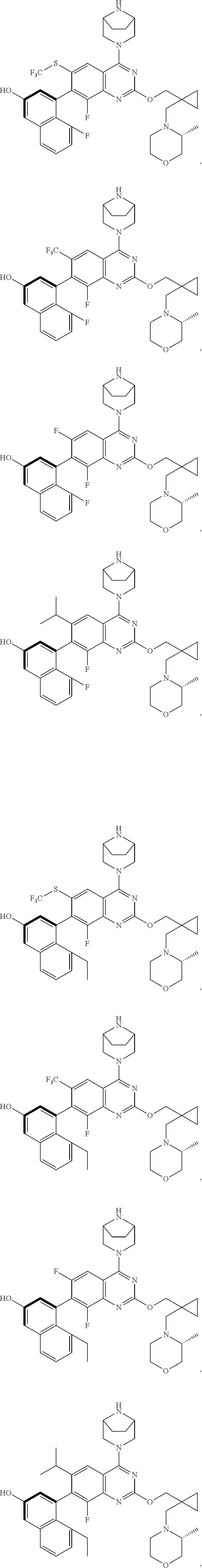

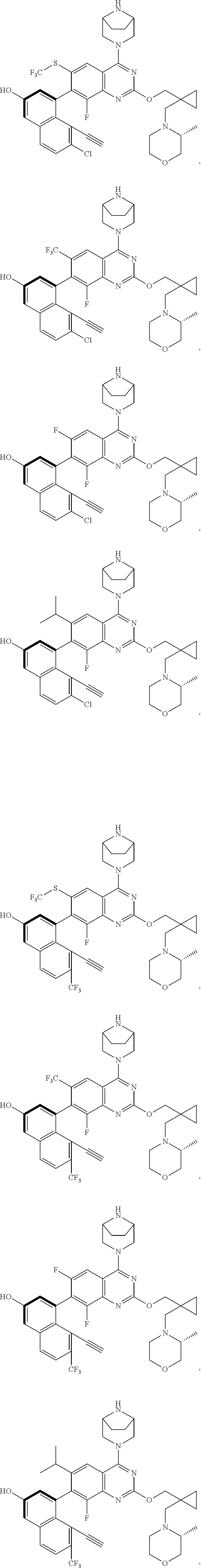

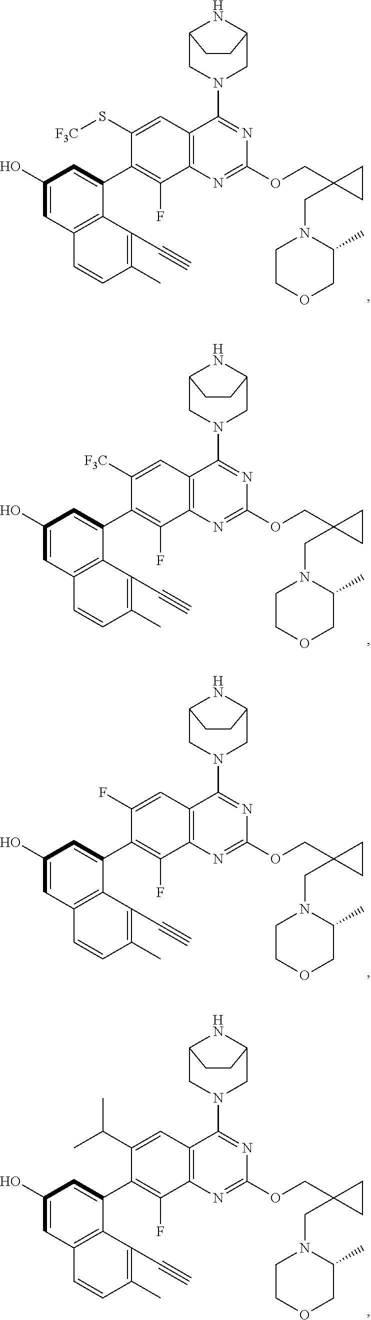

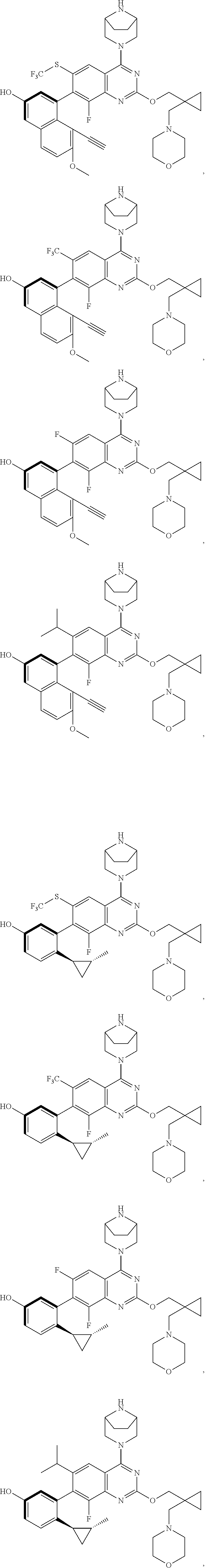

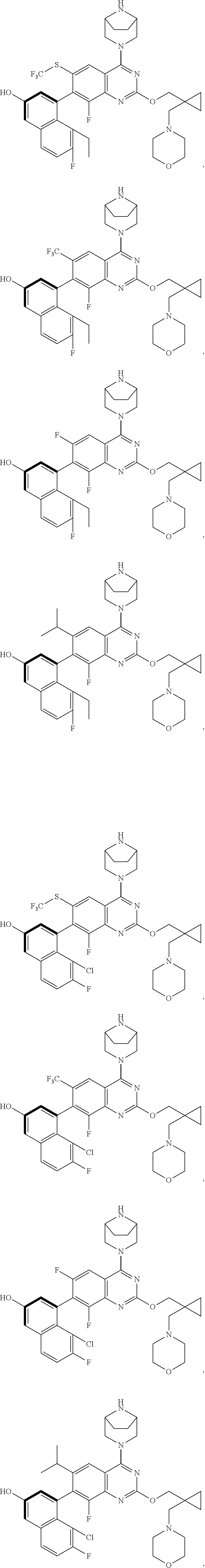

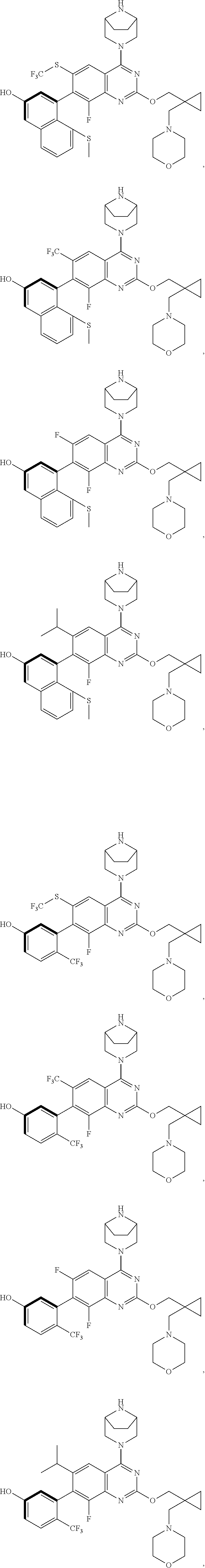

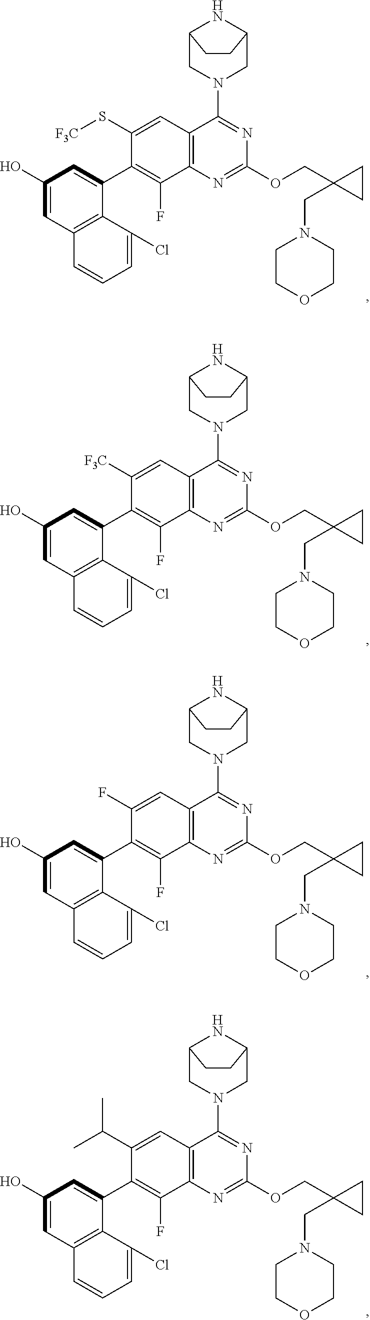

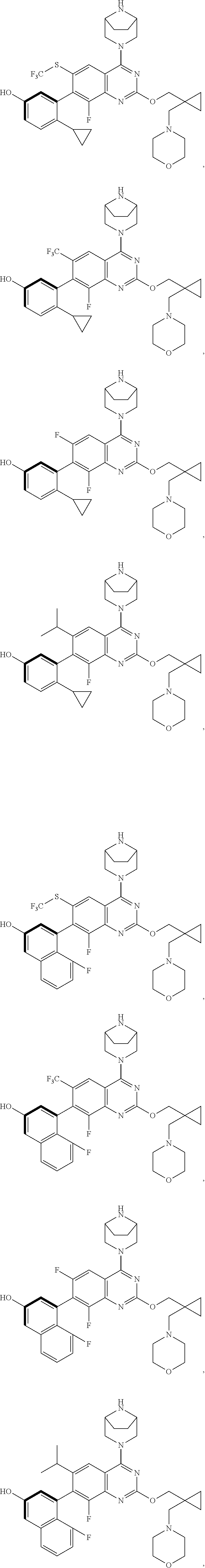

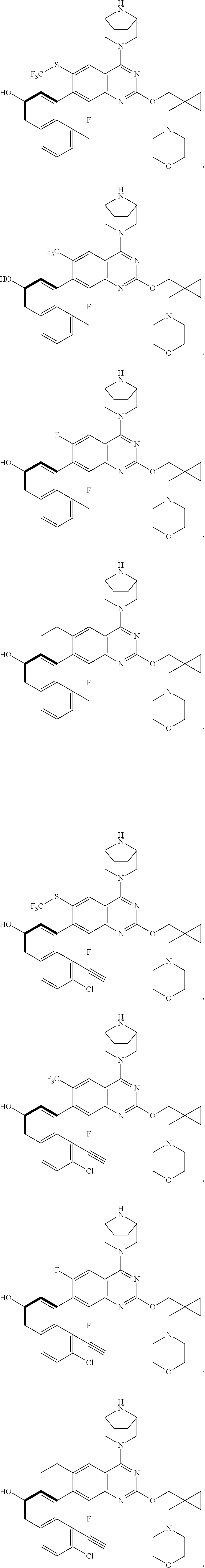

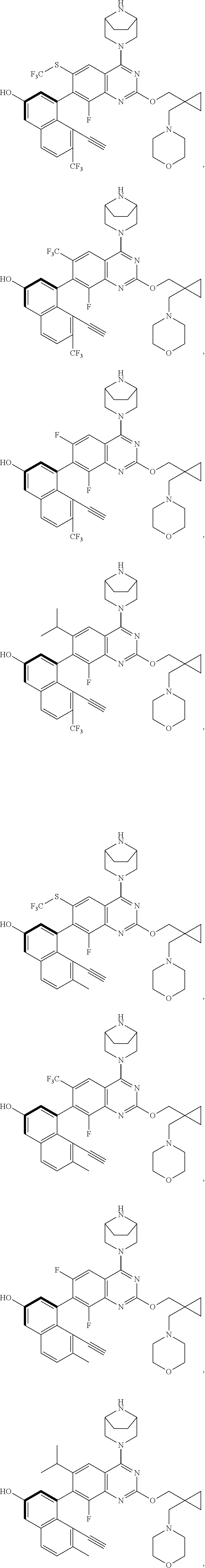

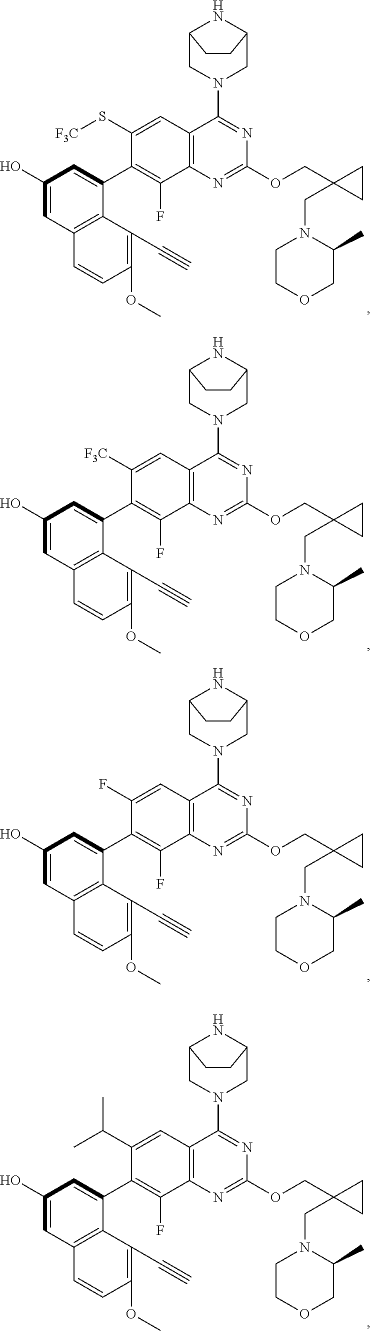

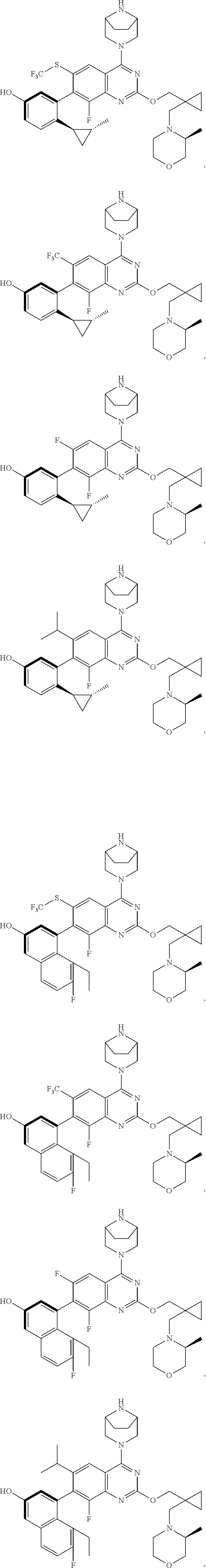

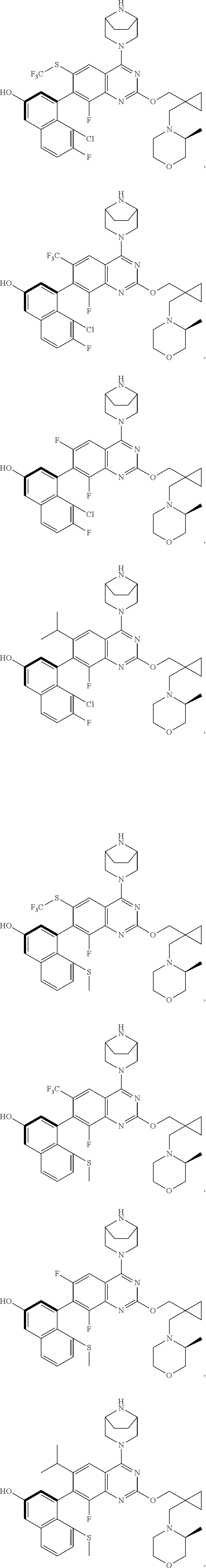

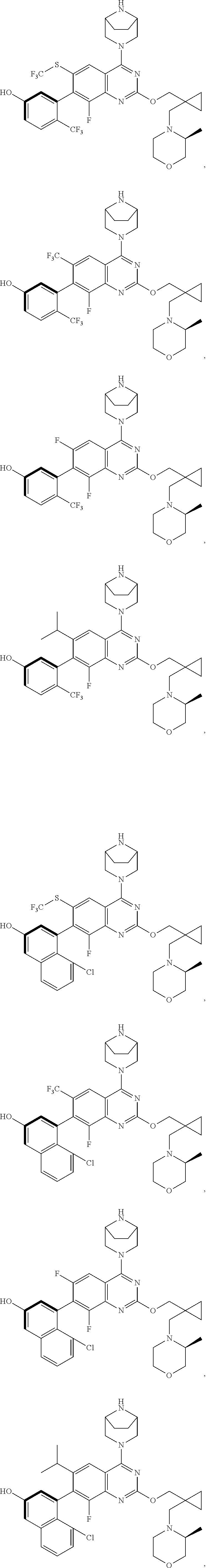

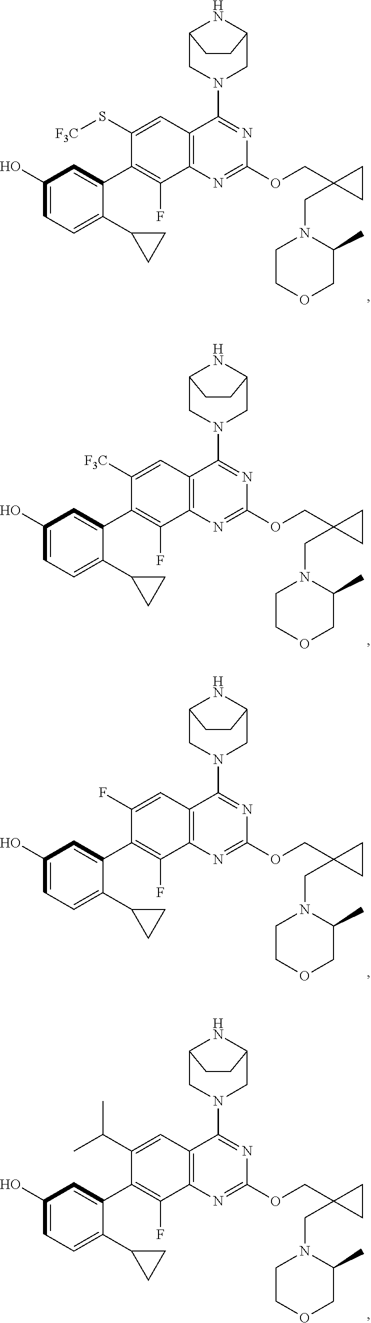

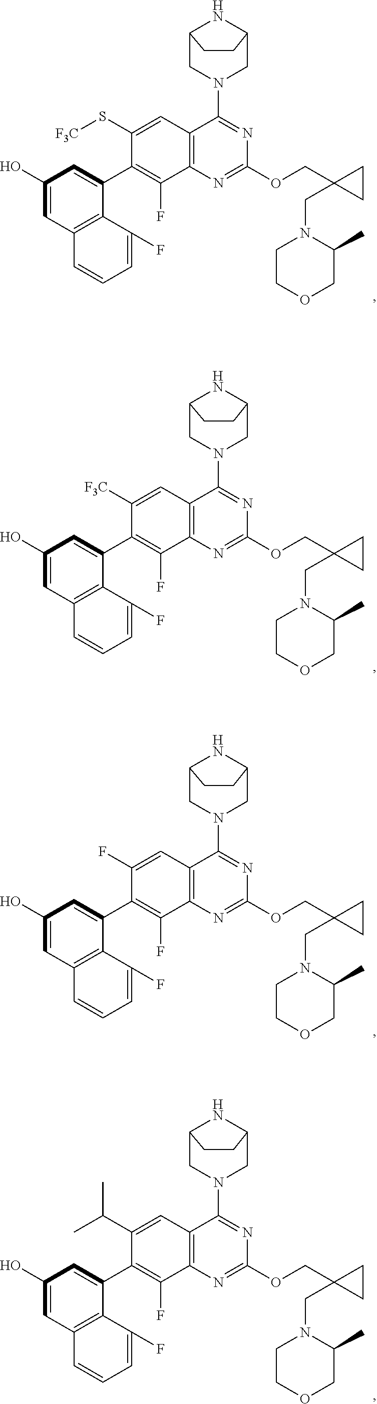

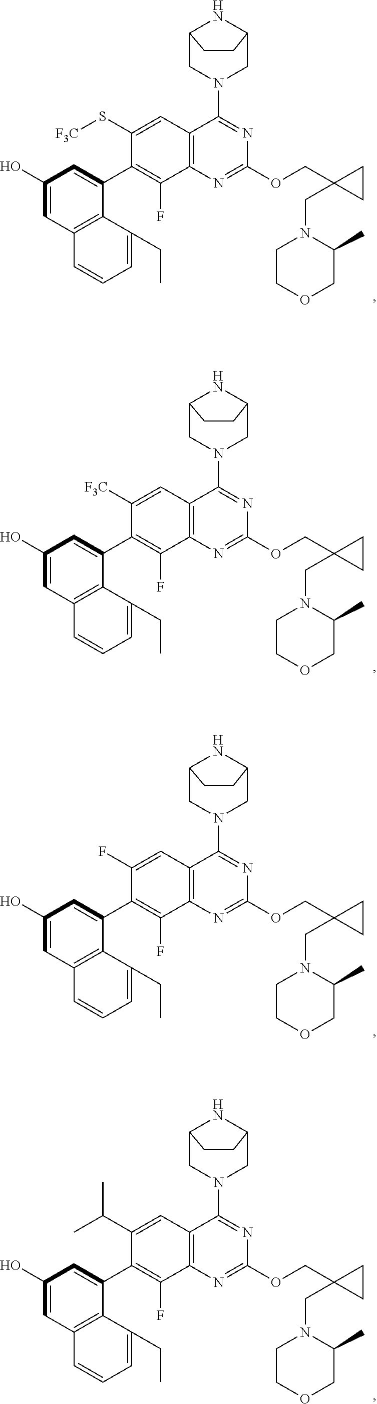

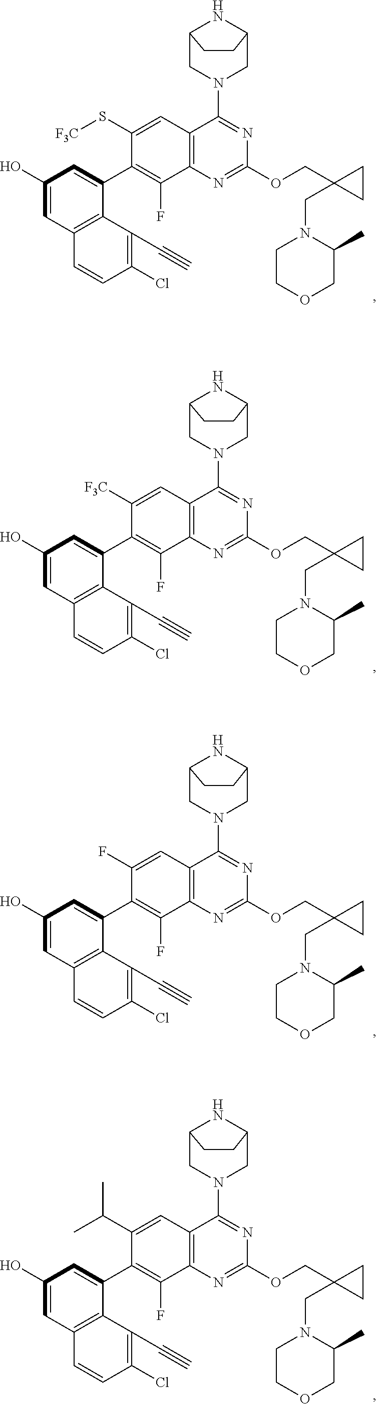

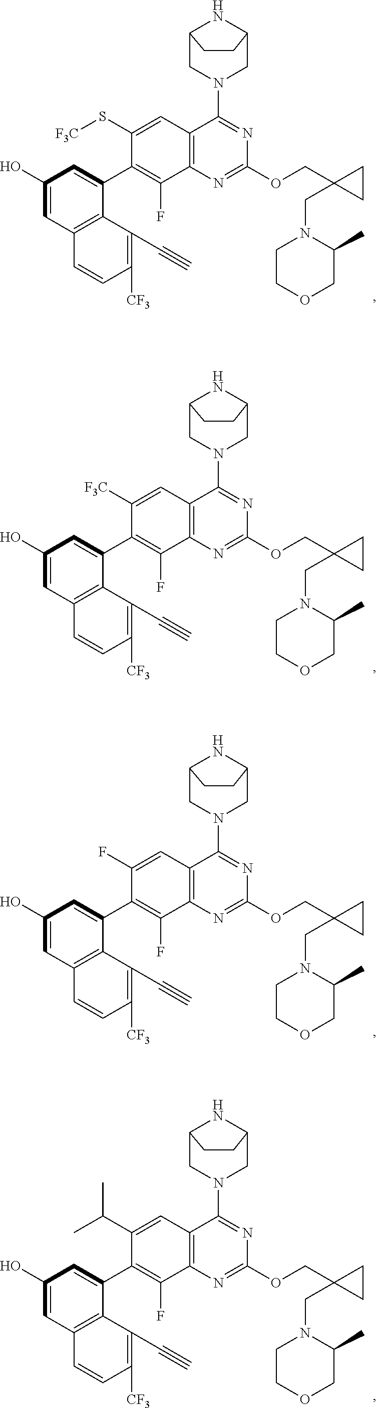

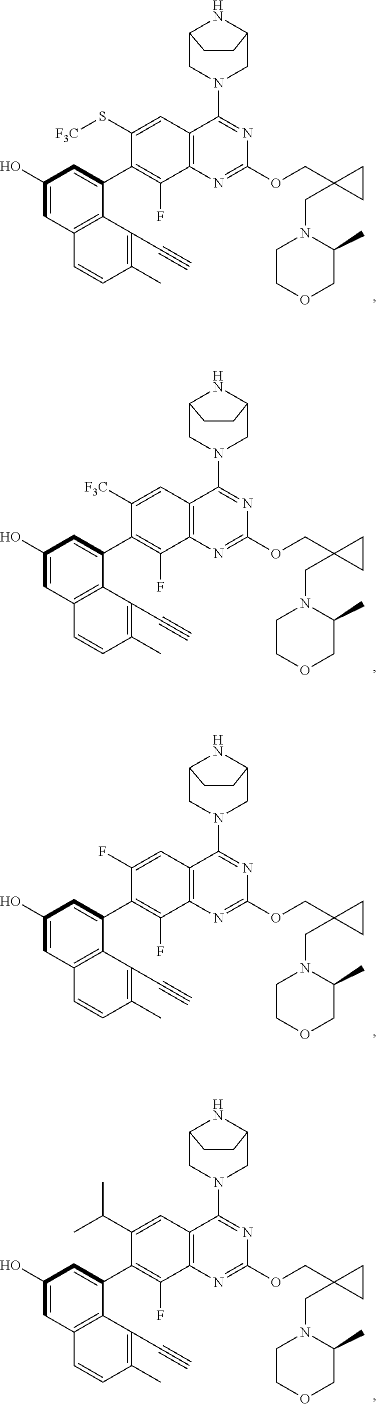

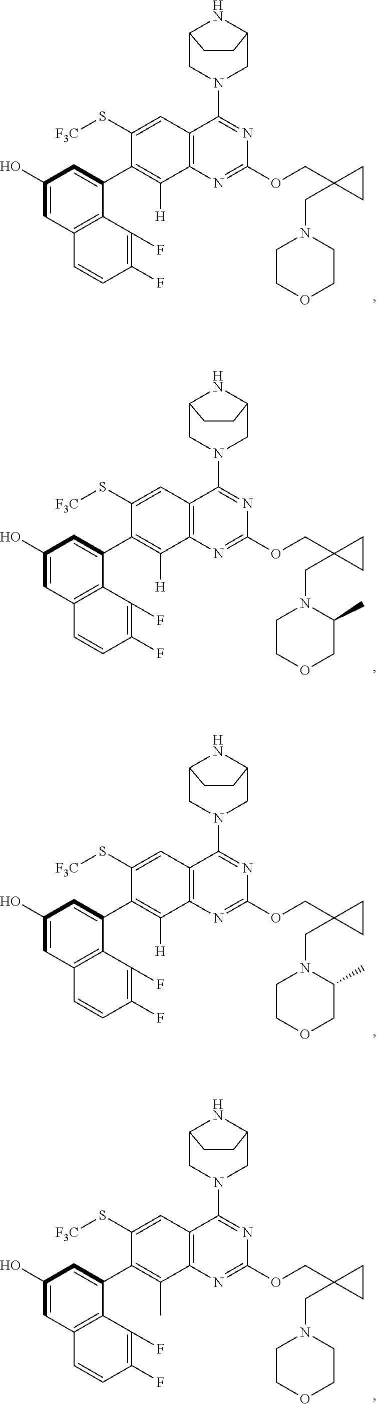

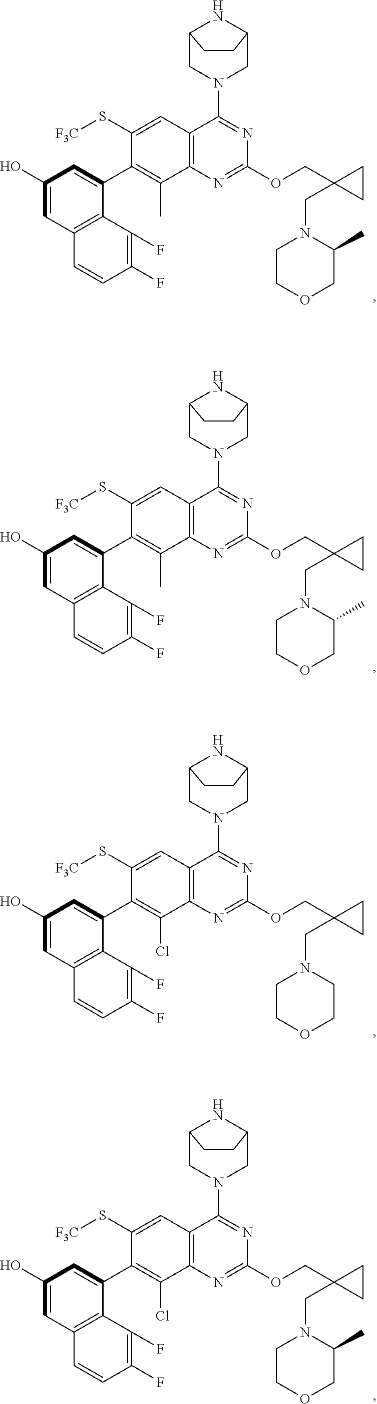

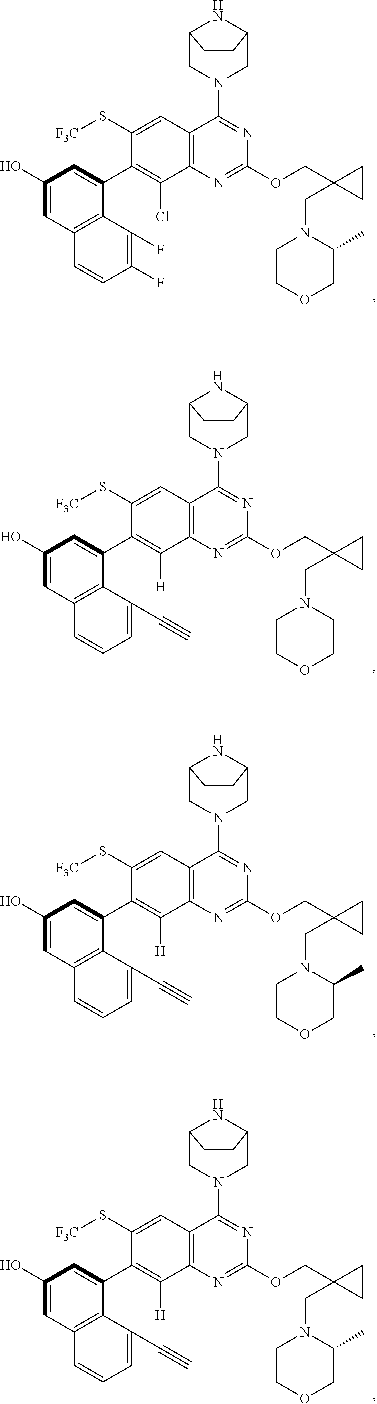

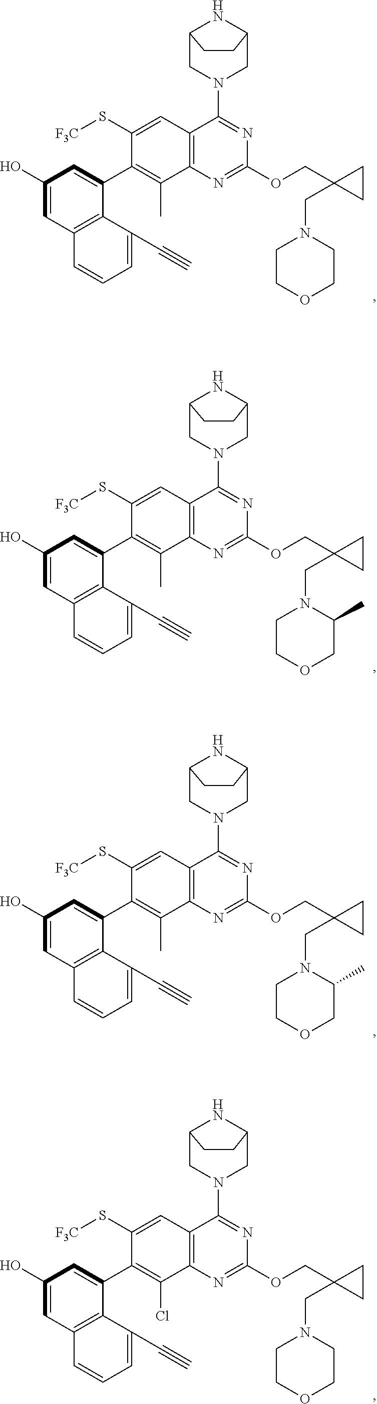

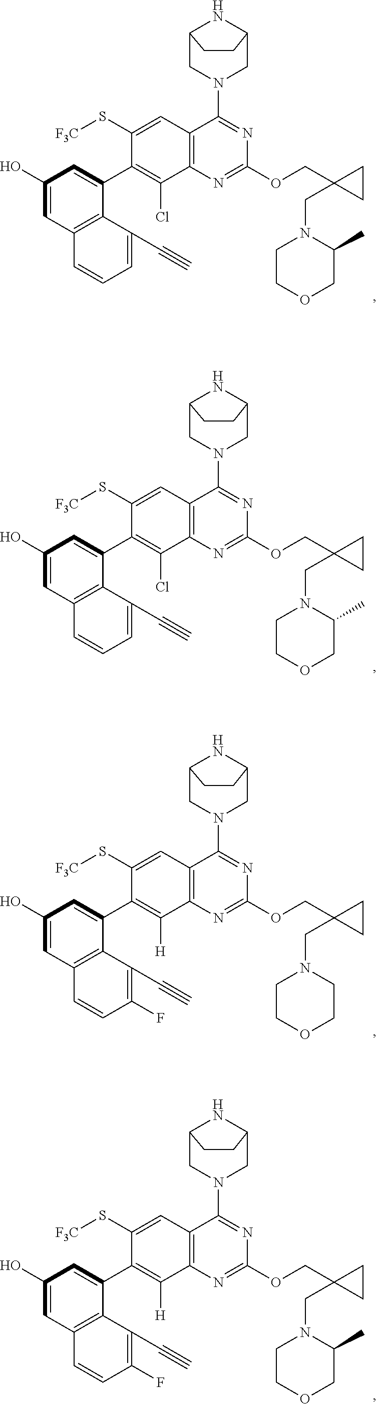

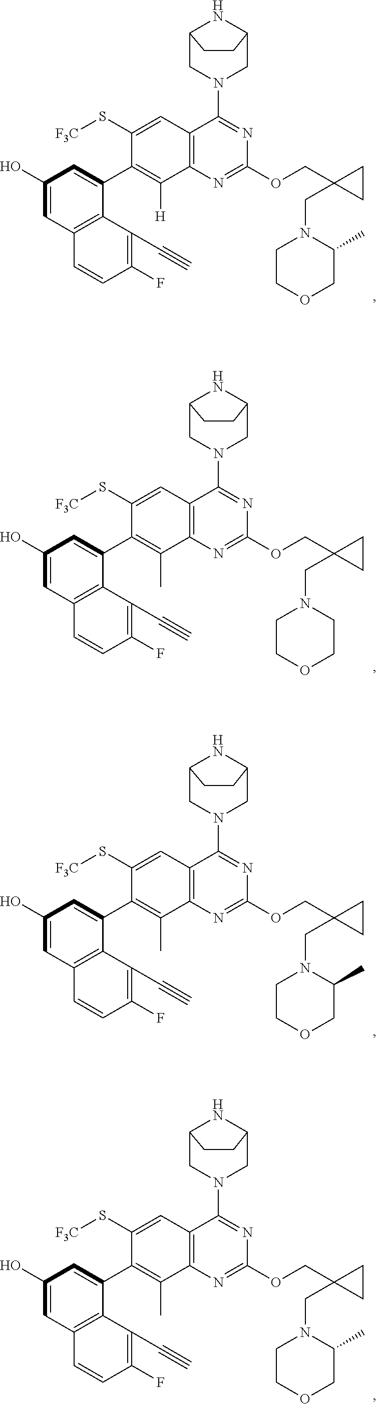

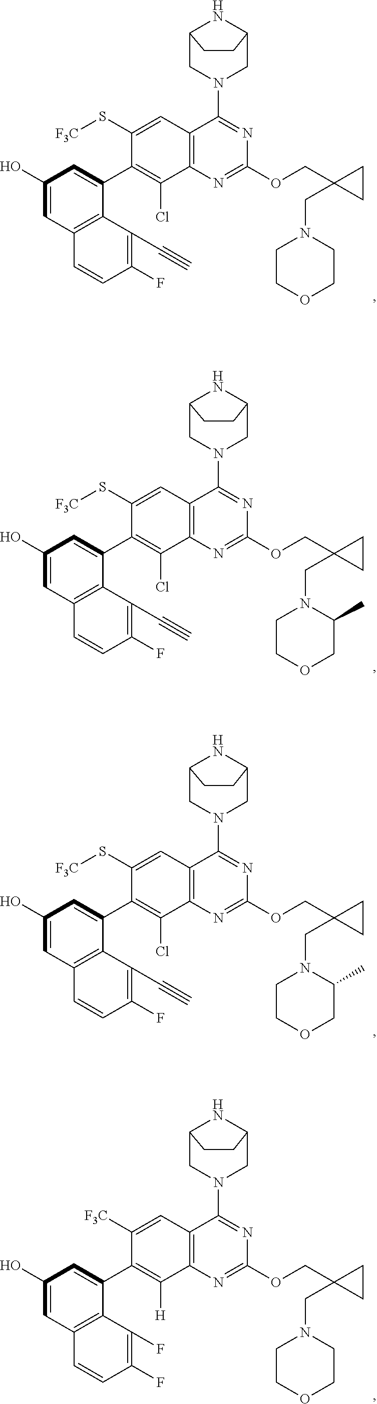

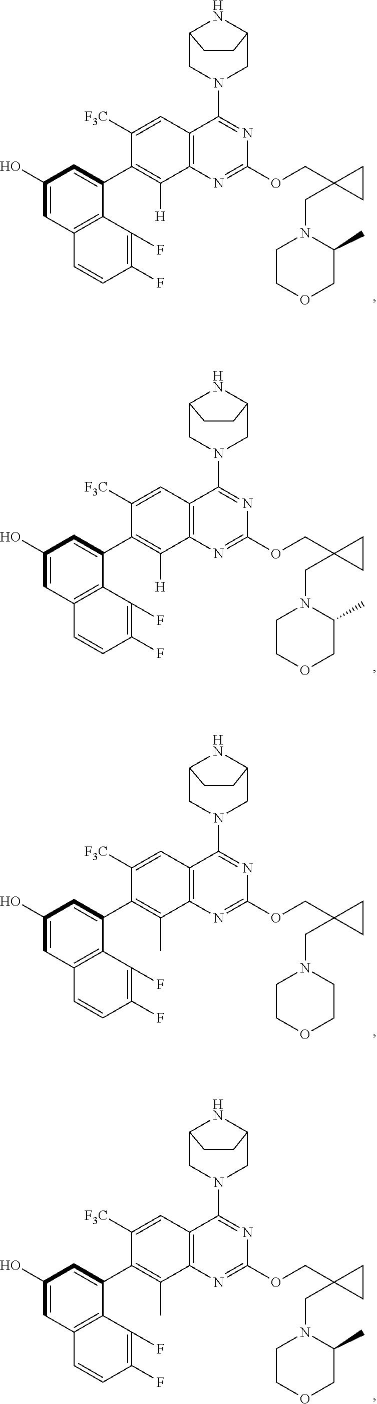

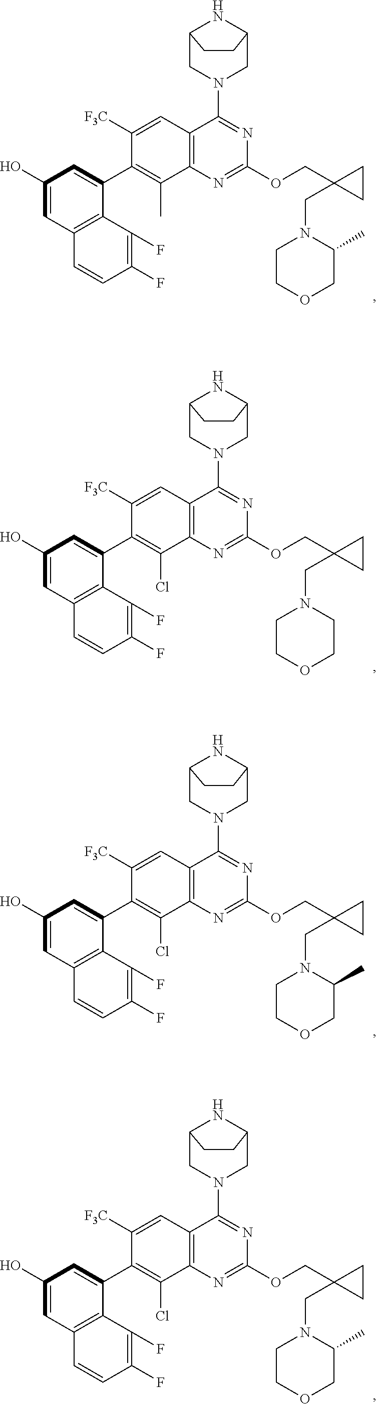

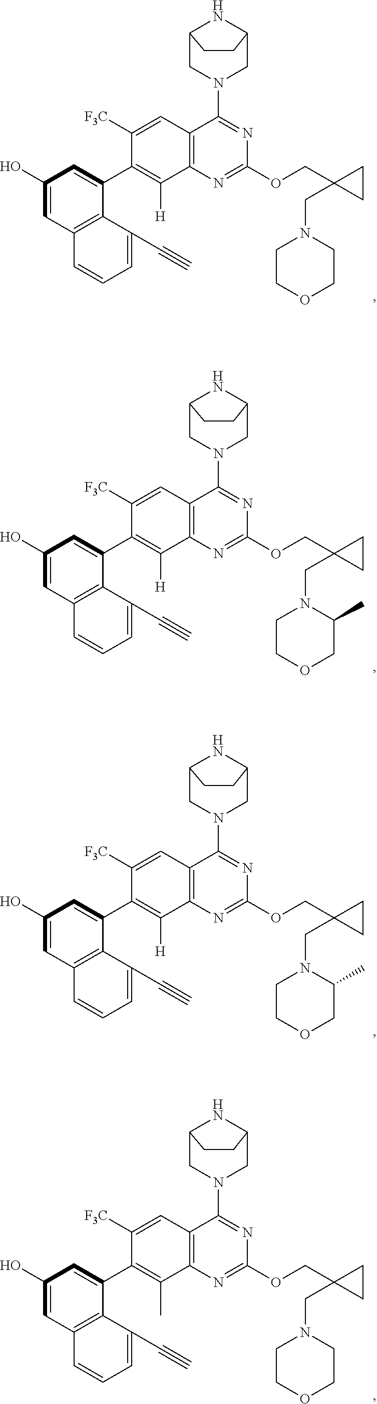

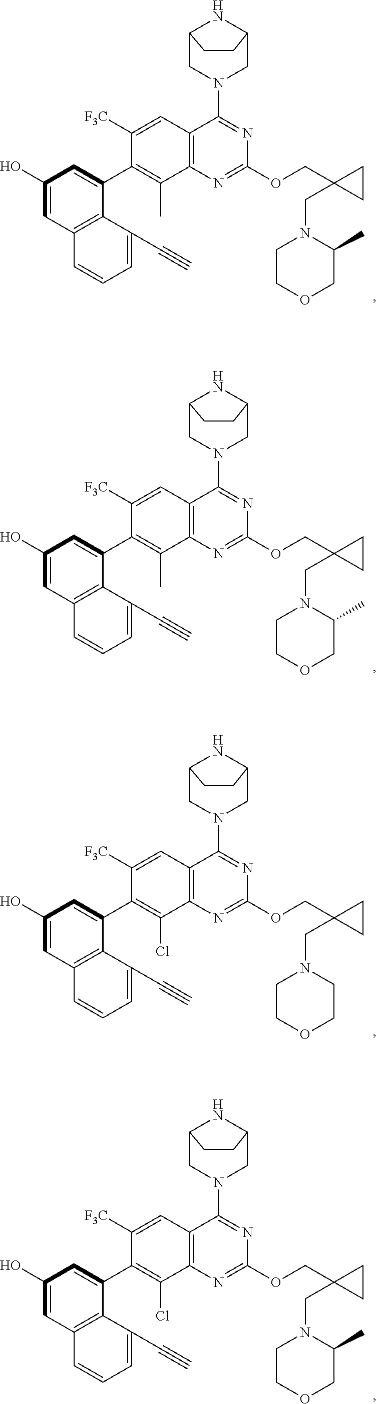

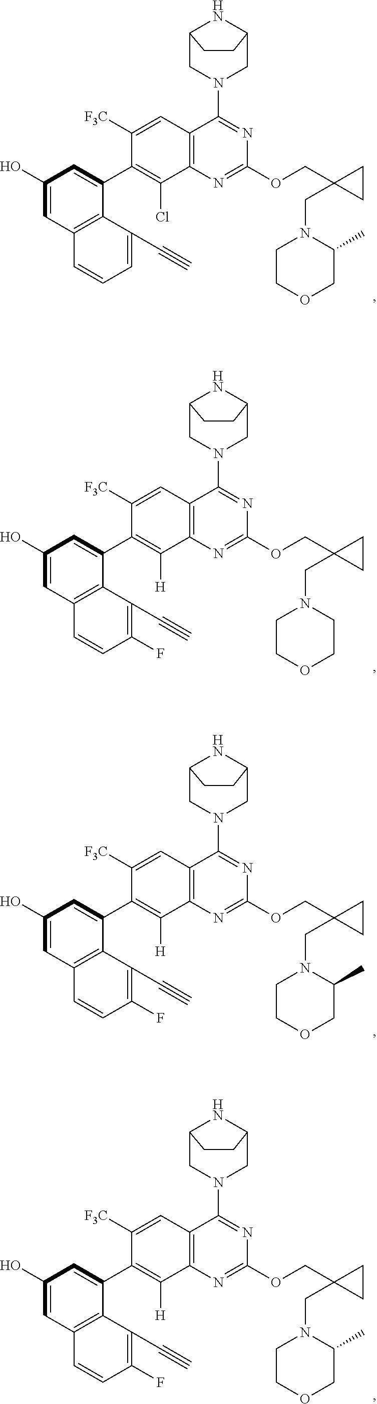

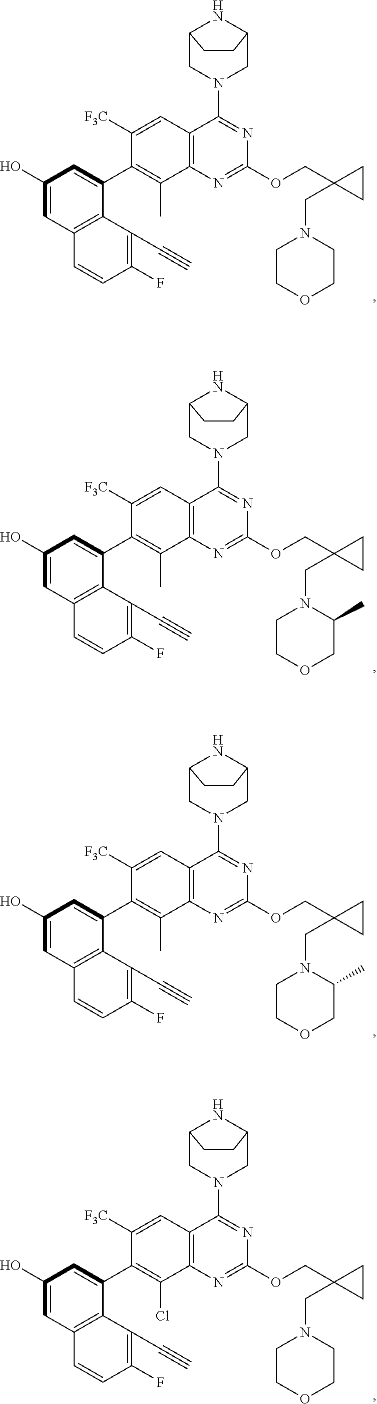

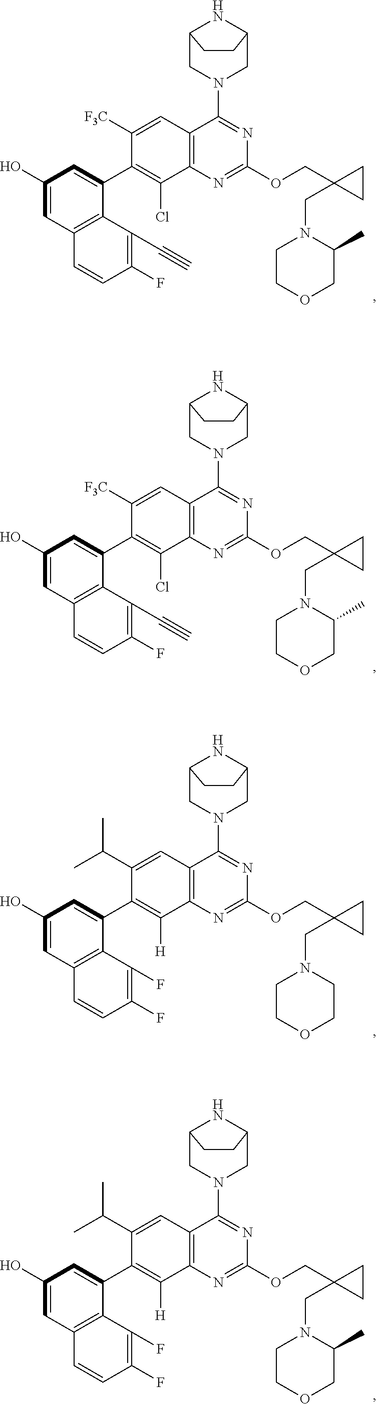

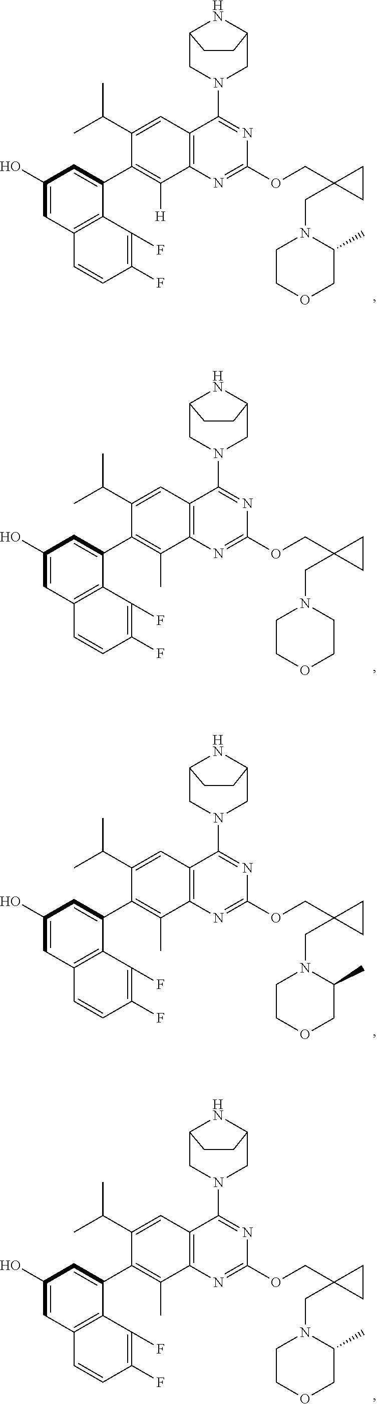

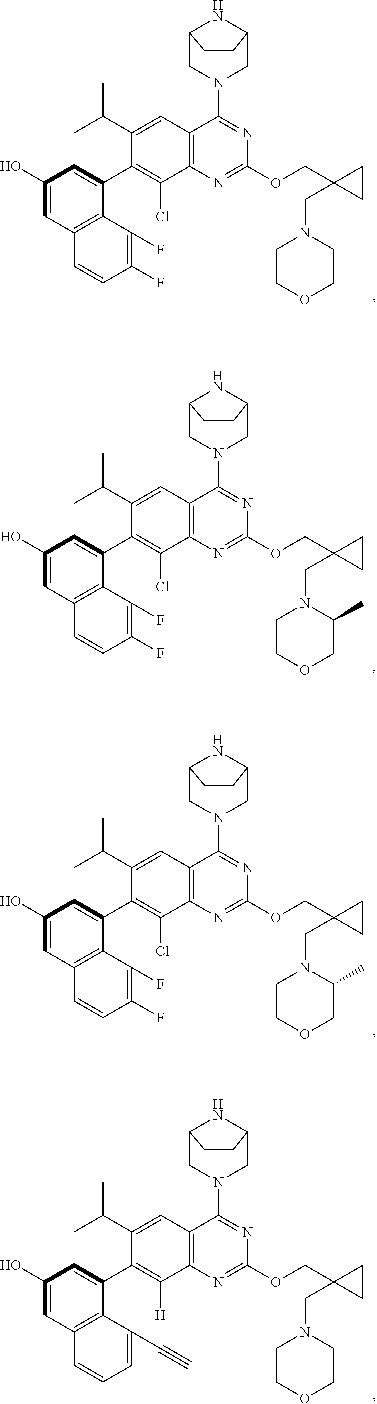

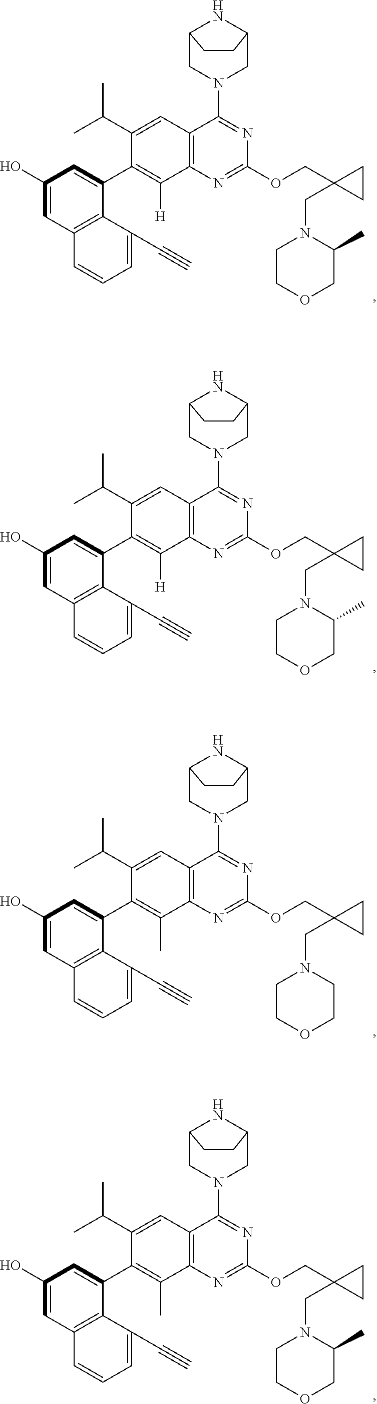

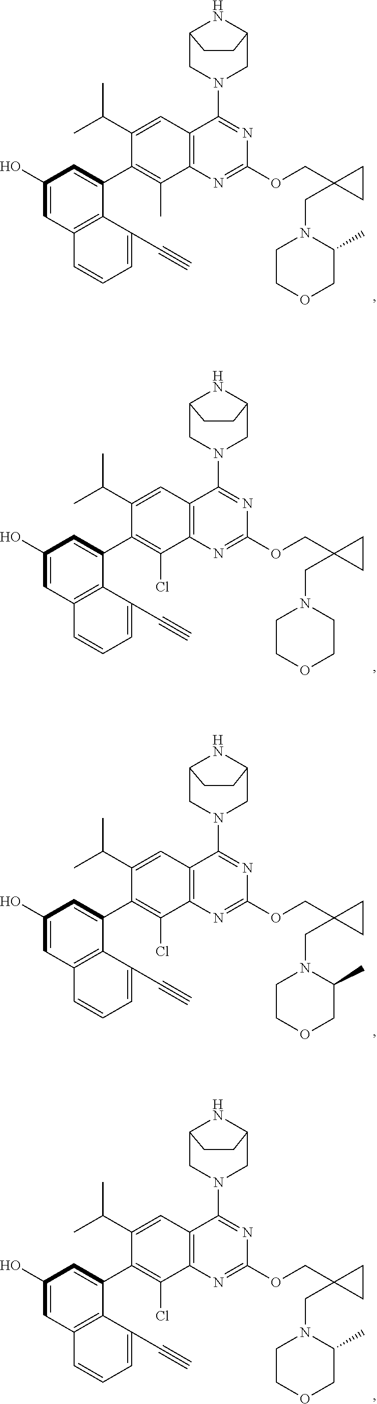

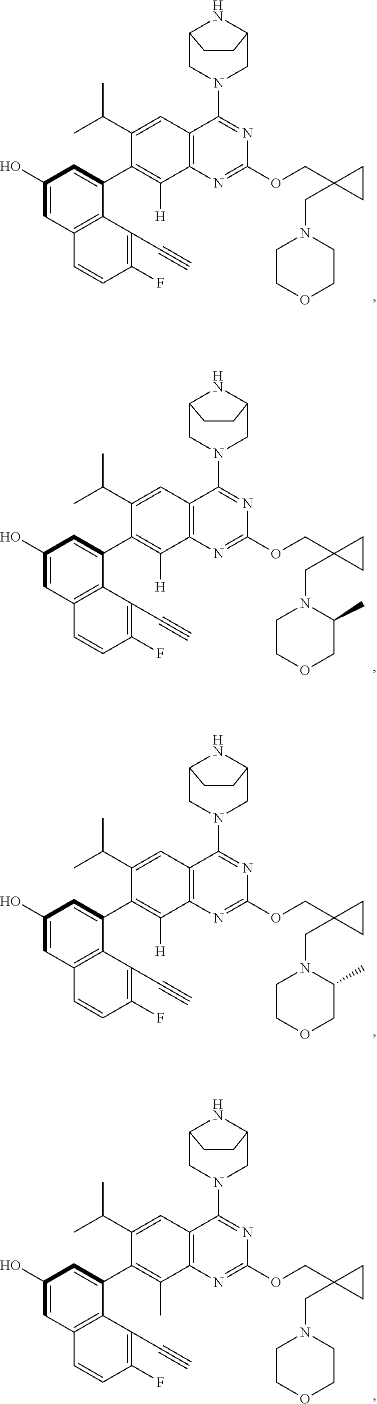

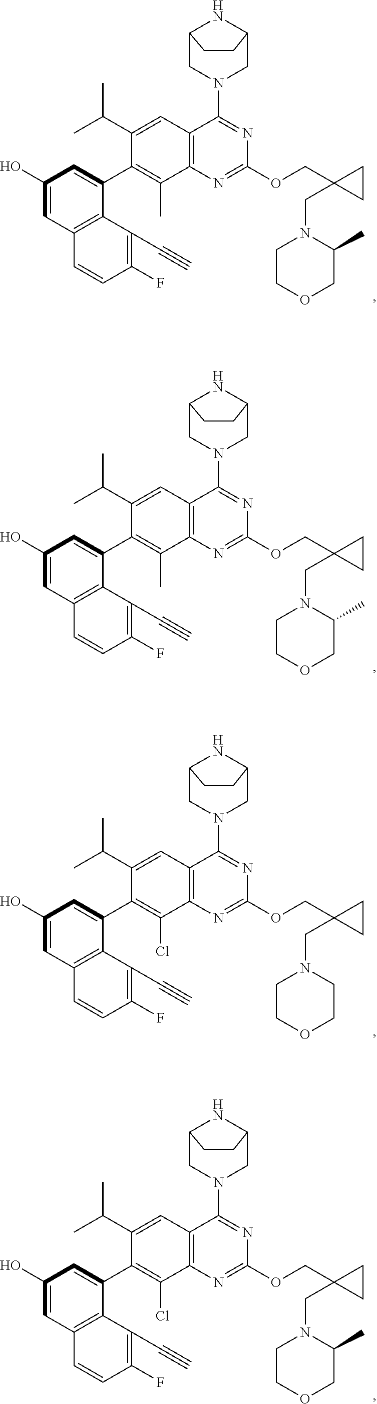

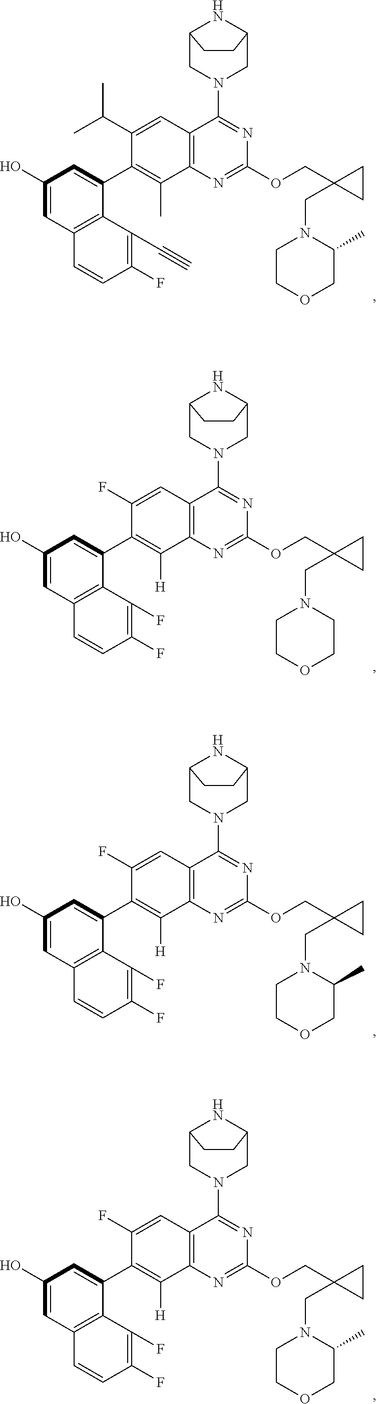

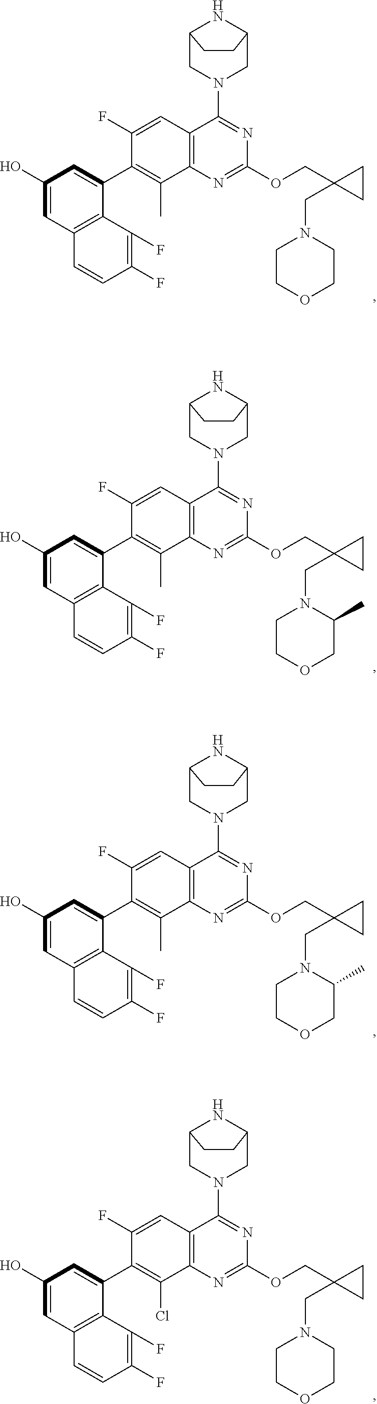

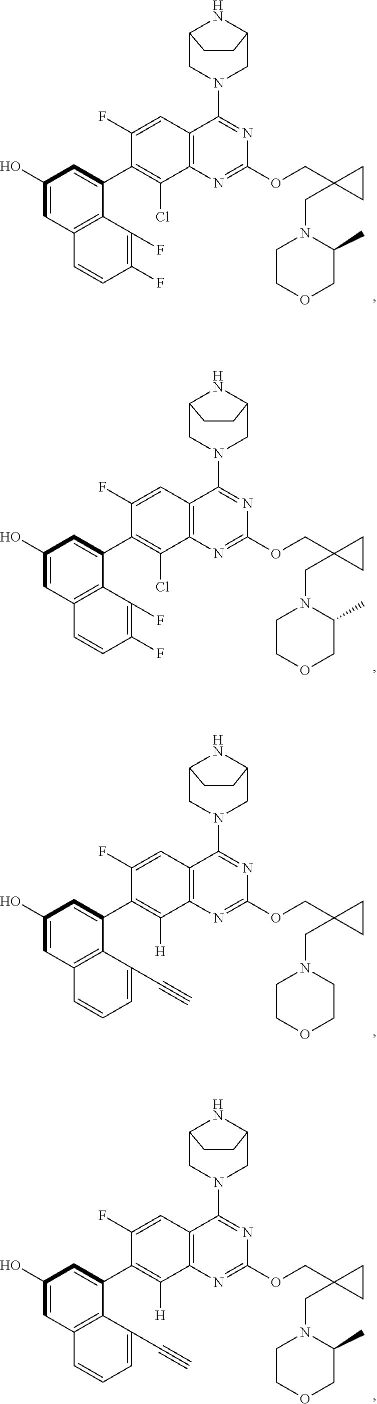

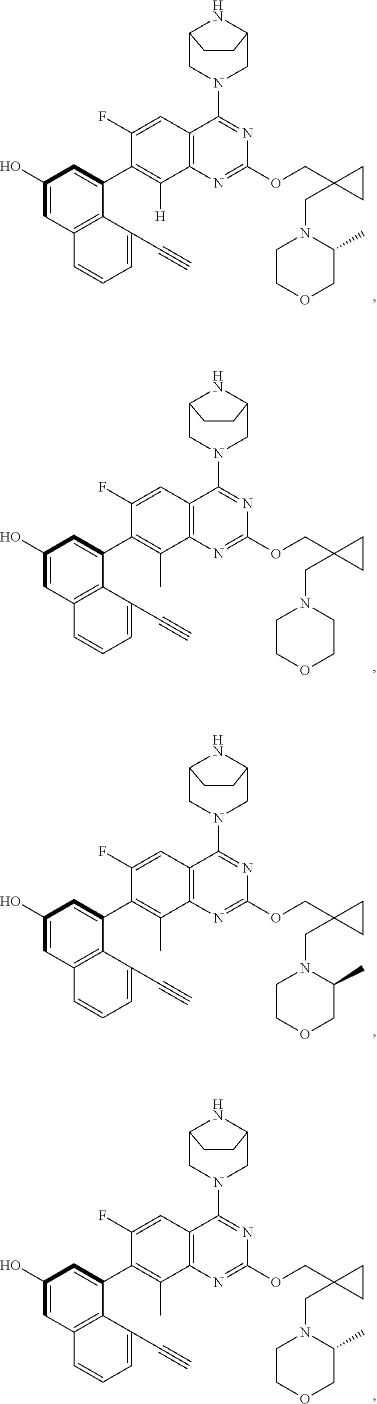

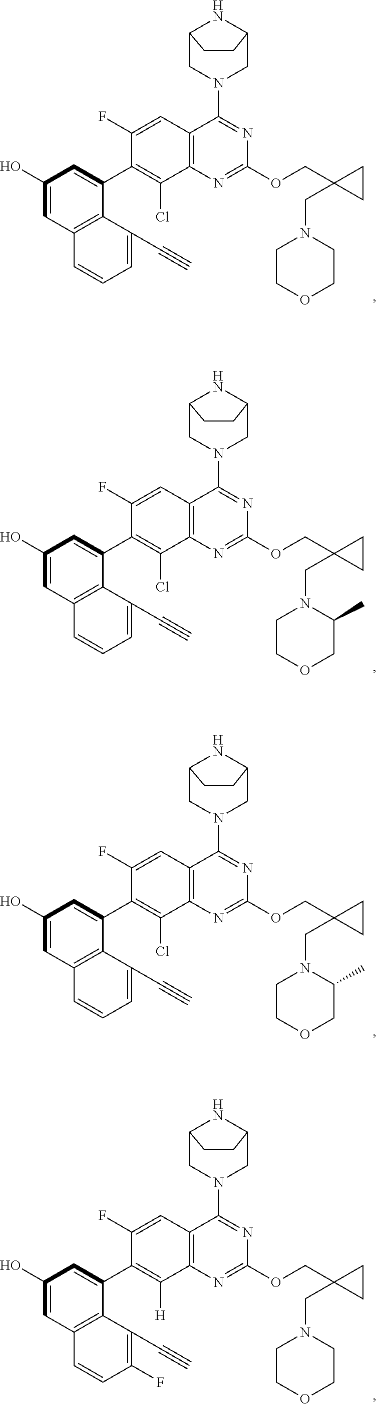

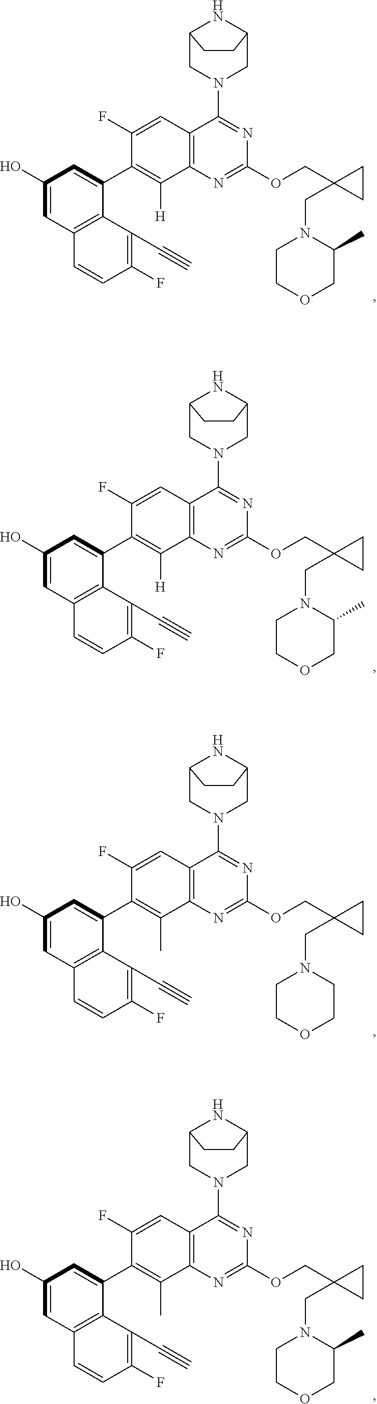

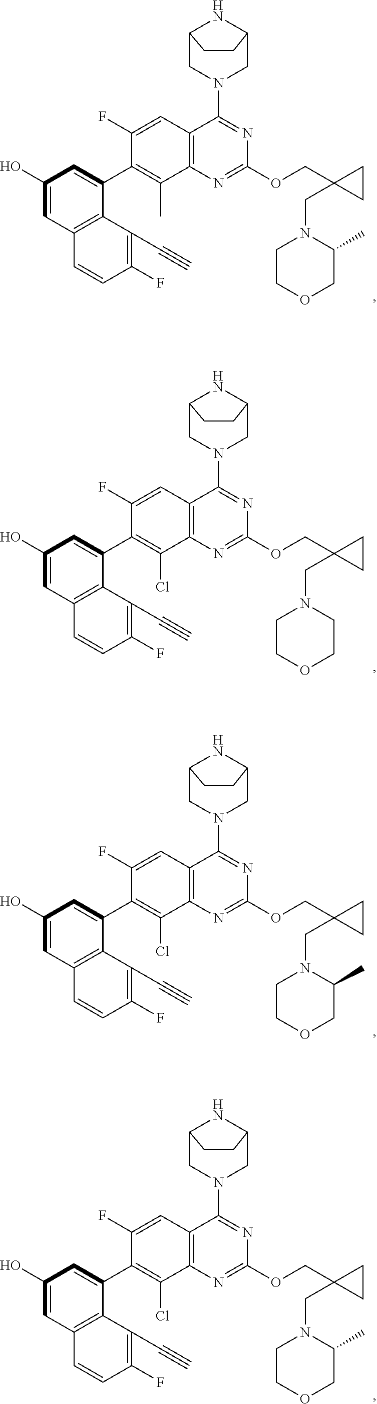

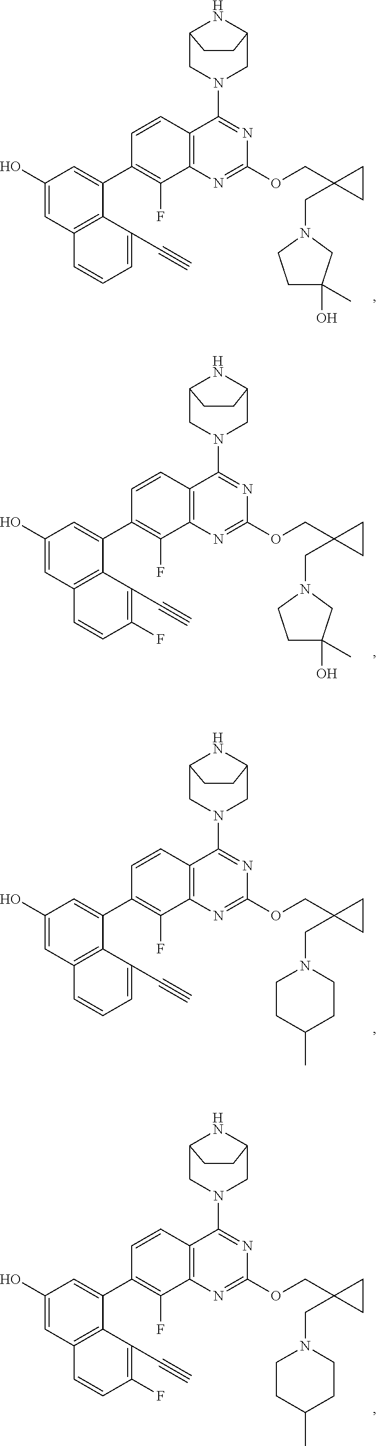 [SMILES: C#Cc1cccc2cc(O)cc(-c3ccc4c(N5CC6CCC(C5)N6)nc(OCC5(CN6CCC(C)(O)C6)CC5)nc4c3F)c12 ; C#Cc1c(F)ccc2cc(O)cc(-c3ccc4c(N5CC6CCC(C5)N6)nc(OCC5(CN6CCC(C)(O)C6)CC5)nc4c3F)c12 ; C#Cc1cccc2cc(O)cc(-c3ccc4c(N5CC6CCC(C5)N6)nc(OCC5(CN6CCC(C)CC6)CC5)nc4c3F)c12 ; C#Cc1c(F)ccc2cc(O)cc(-c3ccc4c(N5CC6CCC(C5)N6)nc(OCC5(CN6CCC(C)CC6)CC5)nc4c3F)c12]

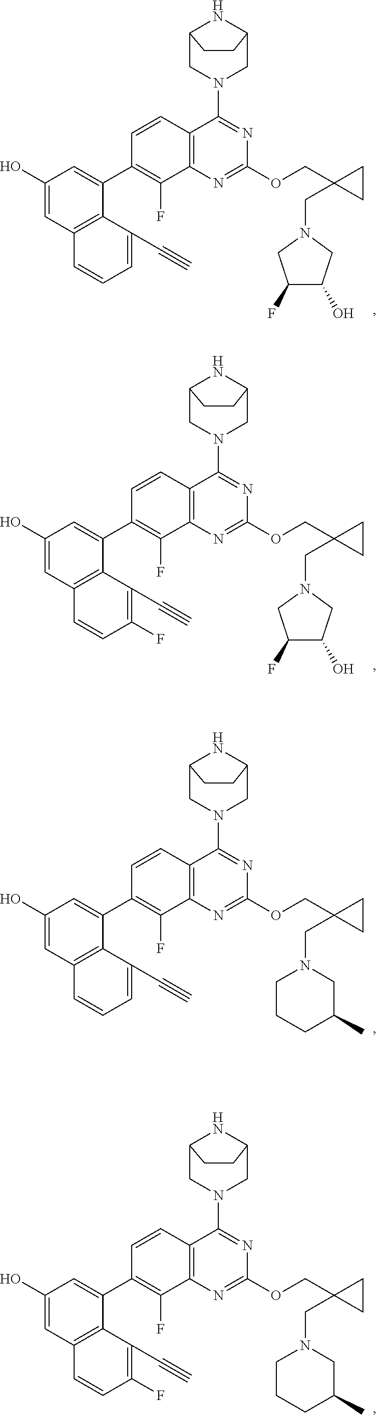 [SMILES: C#Cc1cccc2cc(O)cc(-c3ccc4c(N5CC6CCC(C5)N6)nc(OCC5(CN6C[C@H](O)[C@@H](F)C6)CC5)nc4c3F)c12 ; C#Cc1c(F)ccc2cc(O)cc(-c3ccc4c(N5CC6CCC(C5)N6)nc(OCC5(CN6C[C@H](O)[C@@H](F)C6)CC5)nc4c3F)c12 ; C#Cc1cccc2cc(O)cc(-c3ccc4c(N5CC6CCC(C5)N6)nc(OCC5(CN6CCC[C@H](C)C6)CC5)nc4c3F)c12 ; C#Cc1c(F)ccc2cc(O)cc(-c3ccc4c(N5CC6CCC(C5)N6)nc(OCC5(CN6CCC[C@H](C)C6)CC5)nc4c3F)c12]

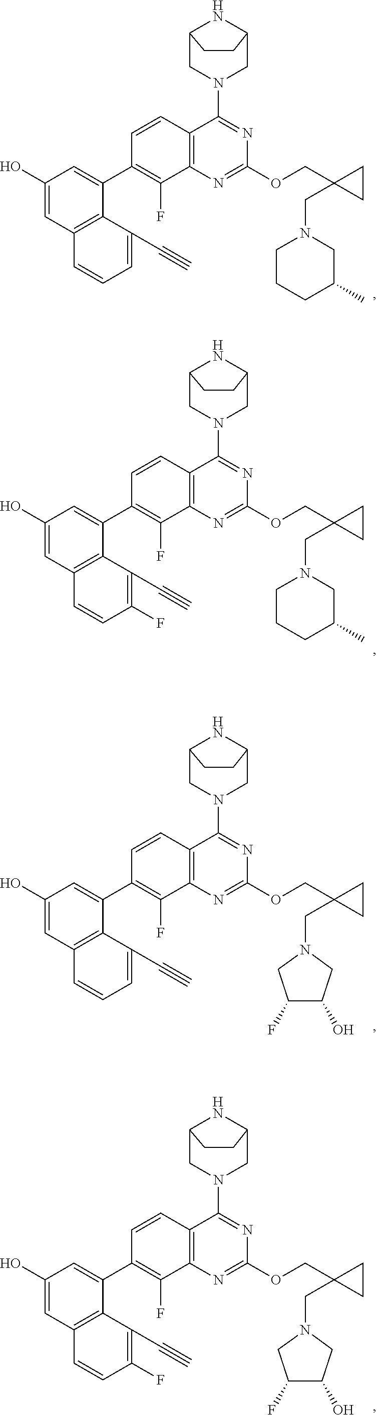 [SMILES: C#Cc1cccc2cc(O)cc(-c3ccc4c(N5CC6CCC(C5)N6)nc(OCC5(CN6CCC[C@@H](C)C6)CC5)nc4c3F)c12 ; C#Cc1c(F)ccc2cc(O)cc(-c3ccc4c(N5CC6CCC(C5)N6)nc(OCC5(CN6CCC[C@@H](C)C6)CC5)nc4c3F)c12 ; C#Cc1cccc2cc(O)cc(-c3ccc4c(N5CC6CCC(C5)N6)nc(OCC5(CN6C[C@@H](F)[C@@H](O)C6)CC5)nc4c3F)c12 ; C#Cc1c(F)ccc2cc(O)cc(-c3ccc4c(N5CC6CCC(C5)N6)nc(OCC5(CN6C[C@@H](F)[C@@H](O)C6)CC5)nc4c3F)c12]

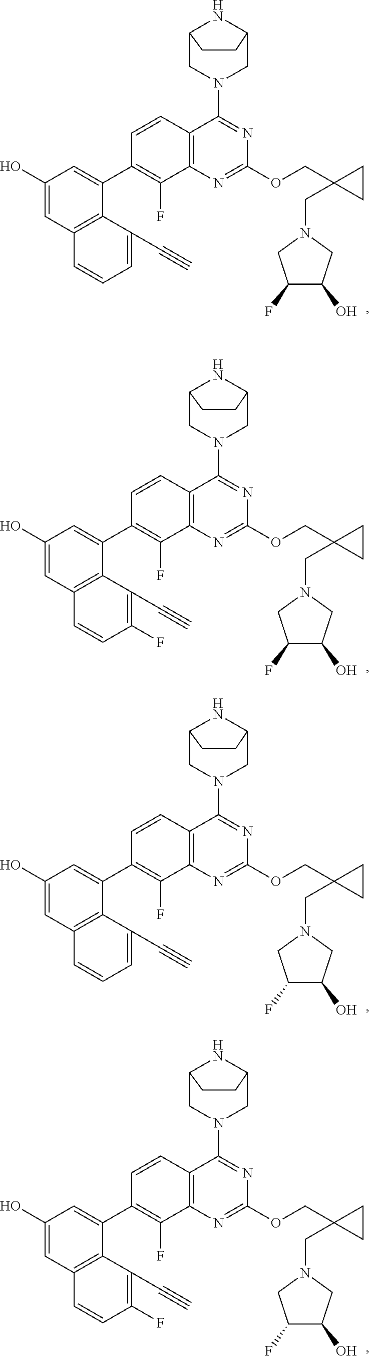 [SMILES: C#Cc1cccc2cc(O)cc(-c3ccc4c(N5CC6CCC(C5)N6)nc(OCC5(CN6C[C@@H](O)[C@@H](F)C6)CC5)nc4c3F)c12 ; C#Cc1c(F)ccc2cc(O)cc(-c3ccc4c(N5CC6CCC(C5)N6)nc(OCC5(CN6C[C@@H](O)[C@@H](F)C6)CC5)nc4c3F)c12 ; C#Cc1cccc2cc(O)cc(-c3ccc4c(N5CC6CCC(C5)N6)nc(OCC5(CN6C[C@@H](O)[C@H](F)C6)CC5)nc4c3F)c12 ; C#Cc1c(F)ccc2cc(O)cc(-c3ccc4c(N5CC6CCC(C5)N6)nc(OCC5(CN6C[C@@H](O)[C@H](F)C6)CC5)nc4c3F)c12]

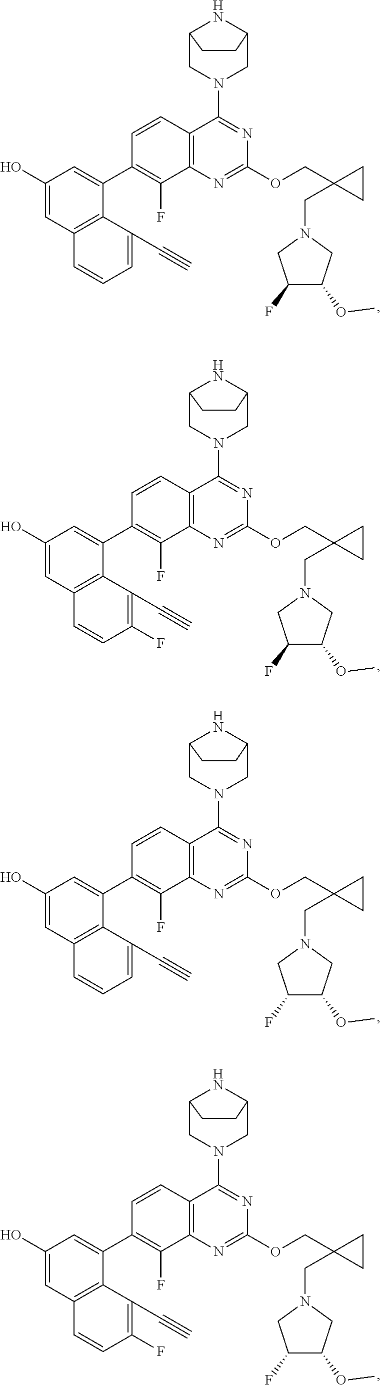 [SMILES: C#Cc1cccc2cc(O)cc(-c3ccc4c(N5CC6CCC(C5)N6)nc(OCC5(CN6C[C@H](OC)[C@@H](F)C6)CC5)nc4c3F)c12 ; C#Cc1c(F)ccc2cc(O)cc(-c3ccc4c(N5CC6CCC(C5)N6)nc(OCC5(CN6C[C@H](OC)[C@@H](F)C6)CC5)nc4c3F)c12 ; C#Cc1cccc2cc(O)cc(-c3ccc4c(N5CC6CCC(C5)N6)nc(OCC5(CN6C[C@@H](F)[C@@H](OC)C6)CC5)nc4c3F)c12 ; C#Cc1c(F)ccc2cc(O)cc(-c3ccc4c(N5CC6CCC(C5)N6)nc(OCC5(CN6C[C@@H](F)[C@@H](OC)C6)CC5)nc4c3F)c12]

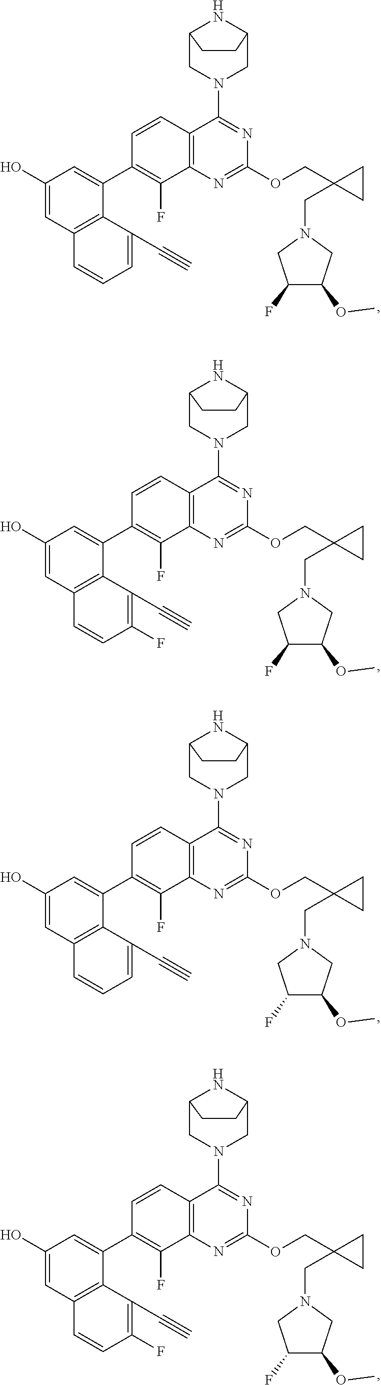 [SMILES: C#Cc1cccc2cc(O)cc(-c3ccc4c(N5CC6CCC(C5)N6)nc(OCC5(CN6C[C@H](F)[C@H](OC)C6)CC5)nc4c3F)c12 ; C#Cc1c(F)ccc2cc(O)cc(-c3ccc4c(N5CC6CCC(C5)N6)nc(OCC5(CN6C[C@H](F)[C@H](OC)C6)CC5)nc4c3F)c12 ; C#Cc1cccc2cc(O)cc(-c3ccc4c(N5CC6CCC(C5)N6)nc(OCC5(CN6C[C@@H](F)[C@H](OC)C6)CC5)nc4c3F)c12 ; C#Cc1c(F)ccc2cc(O)cc(-c3ccc4c(N5CC6CCC(C5)N6)nc(OCC5(CN6C[C@@H](F)[C@H](OC)C6)CC5)nc4c3F)c12]

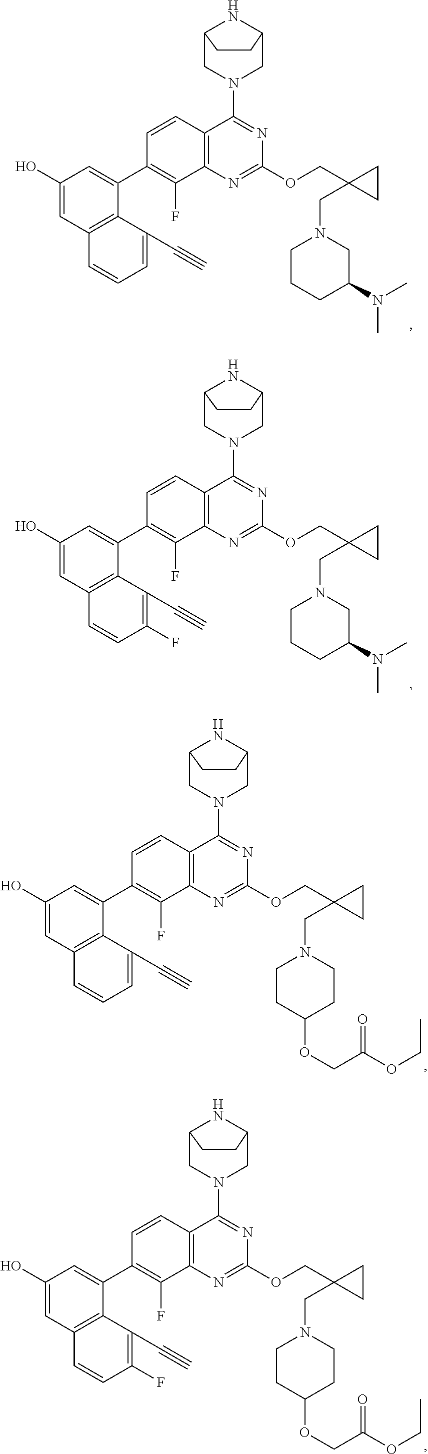 [SMILES: C#Cc1cccc2cc(O)cc(-c3ccc4c(N5CC6CCC(C5)N6)nc(OCC5(CN6CCC[C@H](N(C)C)C6)CC5)nc4c3F)c12 ; C#Cc1c(F)ccc2cc(O)cc(-c3ccc4c(N5CC6CCC(C5)N6)nc(OCC5(CN6CCC[C@H](N(C)C)C6)CC5)nc4c3F)c12 ; C#Cc1cccc2cc(O)cc(-c3ccc4c(N5CC6CCC(C5)N6)nc(OCC5(CN6CCC(OCC(=O)OCC)CC6)CC5)nc4c3F)c12 ; C#Cc1c(F)ccc2cc(O)cc(-c3ccc4c(N5CC6CCC(C5)N6)nc(OCC5(CN6CCC(OCC(=O)OCC)CC6)CC5)nc4c3F)c12]

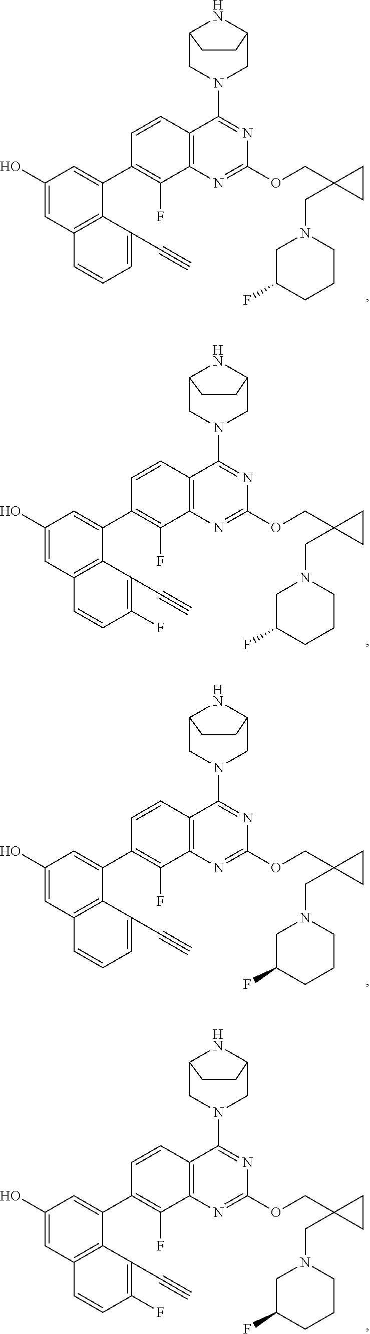 [SMILES: C#Cc1cccc2cc(O)cc(-c3ccc4c(N5CC6CCC(C5)N6)nc(OCC5(CN6CCC[C@H](F)C6)CC5)nc4c3F)c12 ; C#Cc1c(F)ccc2cc(O)cc(-c3ccc4c(N5CC6CCC(C5)N6)nc(OCC5(CN6CCC[C@H](F)C6)CC5)nc4c3F)c12 ; C#Cc1cccc2cc(O)cc(-c3ccc4c(N5CC6CCC(C5)N6)nc(OCC5(CN6CCC[C@@H](F)C6)CC5)nc4c3F)c12 ; C#Cc1c(F)ccc2cc(O)cc(-c3ccc4c(N5CC6CCC(C5)N6)nc(OCC5(CN6CCC[C@@H](F)C6)CC5)nc4c3F)c12]

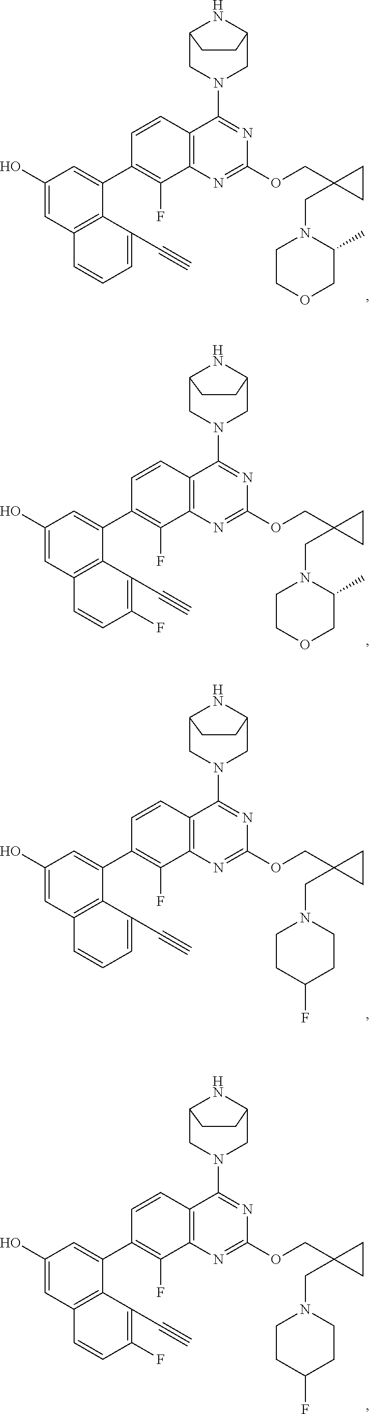 [SMILES: C#Cc1cccc2cc(O)cc(-c3ccc4c(N5CC6CCC(C5)N6)nc(OCC5(CN6CCOC[C@H]6C)CC5)nc4c3F)c12 ; C#Cc1c(F)ccc2cc(O)cc(-c3ccc4c(N5CC6CCC(C5)N6)nc(OCC5(CN6CCOC[C@H]6C)CC5)nc4c3F)c12 ; C#Cc1cccc2cc(O)cc(-c3ccc4c(N5CC6CCC(C5)N6)nc(OCC5(CN6CCC(F)CC6)CC5)nc4c3F)c12 ; C#Cc1c(F)ccc2cc(O)cc(-c3ccc4c(N5CC6CCC(C5)N6)nc(OCC5(CN6CCC(F)CC6)CC5)nc4c3F)c12]

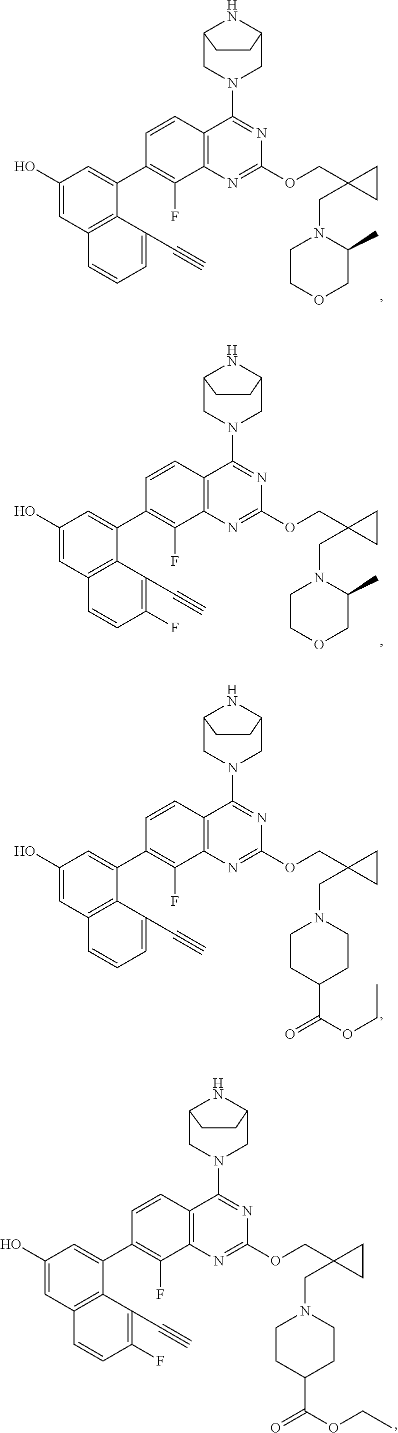 [SMILES: C#Cc1cccc2cc(O)cc(-c3ccc4c(N5CC6CCC(C5)N6)nc(OCC5(CN6CCOC[C@@H]6C)CC5)nc4c3F)c12 ; C#Cc1c(F)ccc2cc(O)cc(-c3ccc4c(N5CC6CCC(C5)N6)nc(OCC5(CN6CCOC[C@@H]6C)CC5)nc4c3F)c12 ; C#Cc1cccc2cc(O)cc(-c3ccc4c(N5CC6CCC(C5)N6)nc(OCC5(CN6CCC(C(=O)OCC)CC6)CC5)nc4c3F)c12 ; C#Cc1c(F)ccc2cc(O)cc(-c3ccc4c(N5CC6CCC(C5)N6)nc(OCC5(CN6CCC(C(=O)OCC)CC6)CC5)nc4c3F)c12]

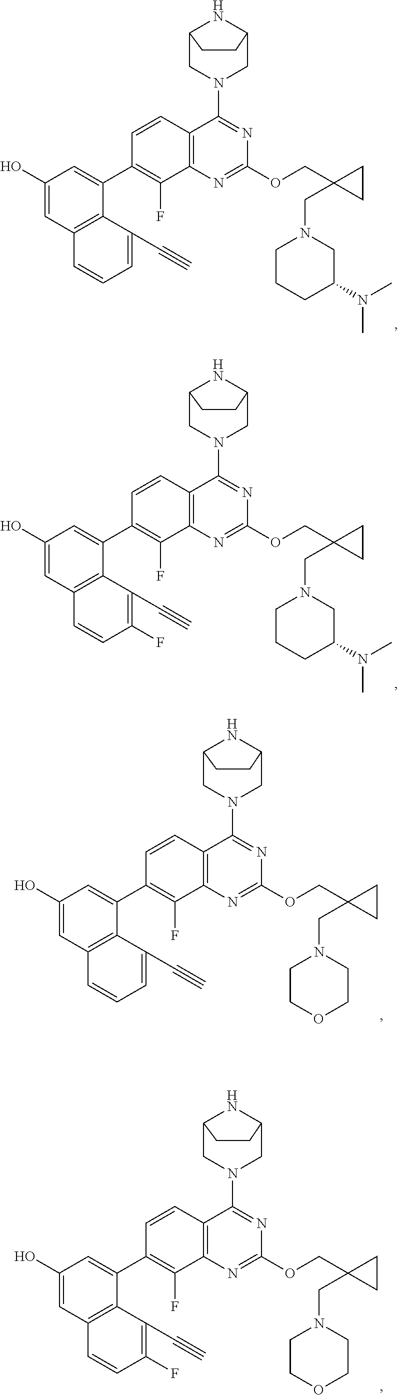 [SMILES: C#Cc1cccc2cc(O)cc(-c3ccc4c(N5CC6CCC(C5)N6)nc(OCC5(CN6CCC[C@@H](N(C)C)C6)CC5)nc4c3F)c12 ; C#Cc1c(F)ccc2cc(O)cc(-c3ccc4c(N5CC6CCC(C5)N6)nc(OCC5(CN6CCC[C@@H](N(C)C)C6)CC5)nc4c3F)c12 ; C#Cc1cccc2cc(O)cc(-c3ccc4c(N5CC6CCC(C5)N6)nc(OCC5(CN6CCOCC6)CC5)nc4c3F)c12 ; C#Cc1c(F)ccc2cc(O)cc(-c3ccc4c(N5CC6CCC(C5)N6)nc(OCC5(CN6CCOCC6)CC5)nc4c3F)c12]

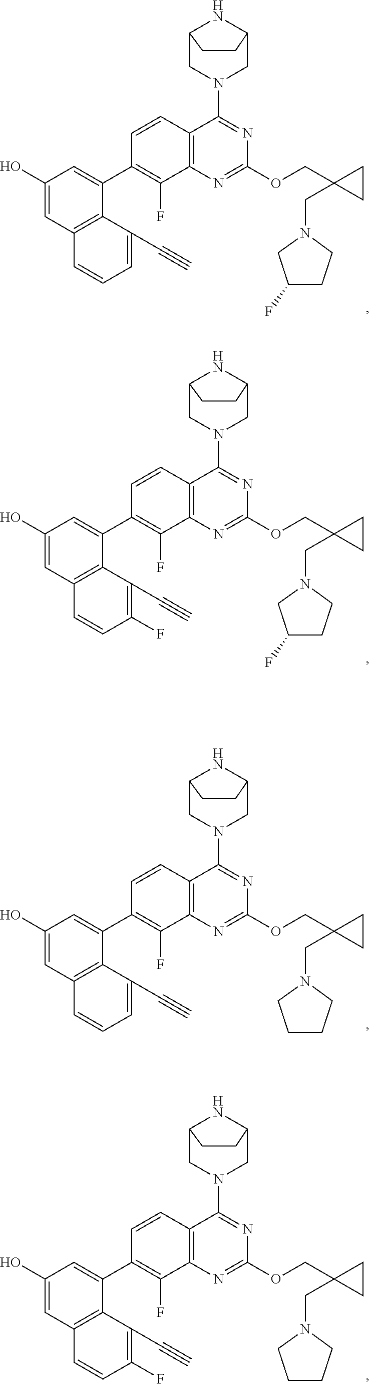 [SMILES: C#Cc1cccc2cc(O)cc(-c3ccc4c(N5CC6CCC(C5)N6)nc(OCC5(CN6CC[C@H](F)C6)CC5)nc4c3F)c12 ; C#Cc1c(F)ccc2cc(O)cc(-c3ccc4c(N5CC6CCC(C5)N6)nc(OCC5(CN6CC[C@H](F)C6)CC5)nc4c3F)c12 ; C#Cc1cccc2cc(O)cc(-c3ccc4c(N5CC6CCC(C5)N6)nc(OCC5(CN6CCCC6)CC5)nc4c3F)c12 ; C#Cc1c(F)ccc2cc(O)cc(-c3ccc4c(N5CC6CCC(C5)N6)nc(OCC5(CN6CCCC6)CC5)nc4c3F)c12]

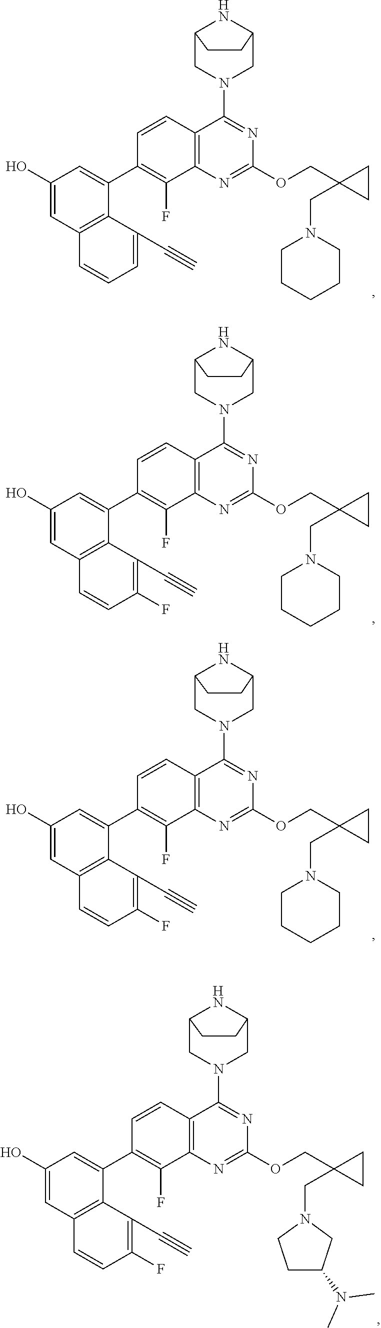 [SMILES: C#Cc1cccc2cc(O)cc(-c3ccc4c(N5CC6CCC(C5)N6)nc(OCC5(CN6CCCCC6)CC5)nc4c3F)c12 ; C#Cc1c(F)ccc2cc(O)cc(-c3ccc4c(N5CC6CCC(C5)N6)nc(OCC5(CN6CCCCC6)CC5)nc4c3F)c12 ; C#Cc1c(F)ccc2cc(O)cc(-c3ccc4c(N5CC6CCC(C5)N6)nc(OCC5(CN6CCCCC6)CC5)nc4c3F)c12 ; C#Cc1c(F)ccc2cc(O)cc(-c3ccc4c(N5CC6CCC(C5)N6)nc(OCC5(CN6CC[C@@H](N(C)C)C6)CC5)nc4c3F)c12]

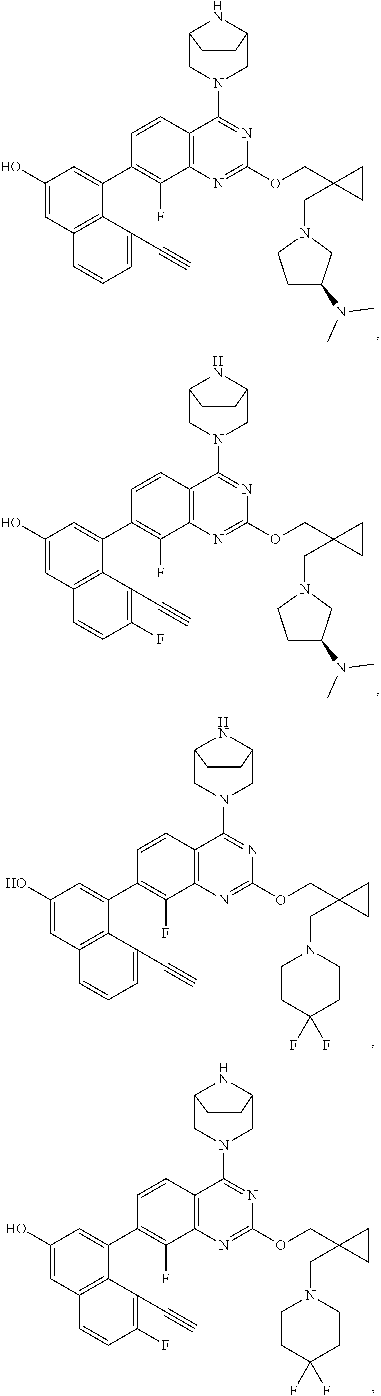 [SMILES: C#Cc1cccc2cc(O)cc(-c3ccc4c(N5CC6CCC(C5)N6)nc(OCC5(CN6CC[C@H](N(C)C)C6)CC5)nc4c3F)c12 ; C#Cc1c(F)ccc2cc(O)cc(-c3ccc4c(N5CC6CCC(C5)N6)nc(OCC5(CN6CC[C@H](N(C)C)C6)CC5)nc4c3F)c12 ; C#Cc1cccc2cc(O)cc(-c3ccc4c(N5CC6CCC(C5)N6)nc(OCC5(CN6CCC(F)(F)CC6)CC5)nc4c3F)c12 ; C#Cc1c(F)ccc2cc(O)cc(-c3ccc4c(N5CC6CCC(C5)N6)nc(OCC5(CN6CCC(F)(F)CC6)CC5)nc4c3F)c12]

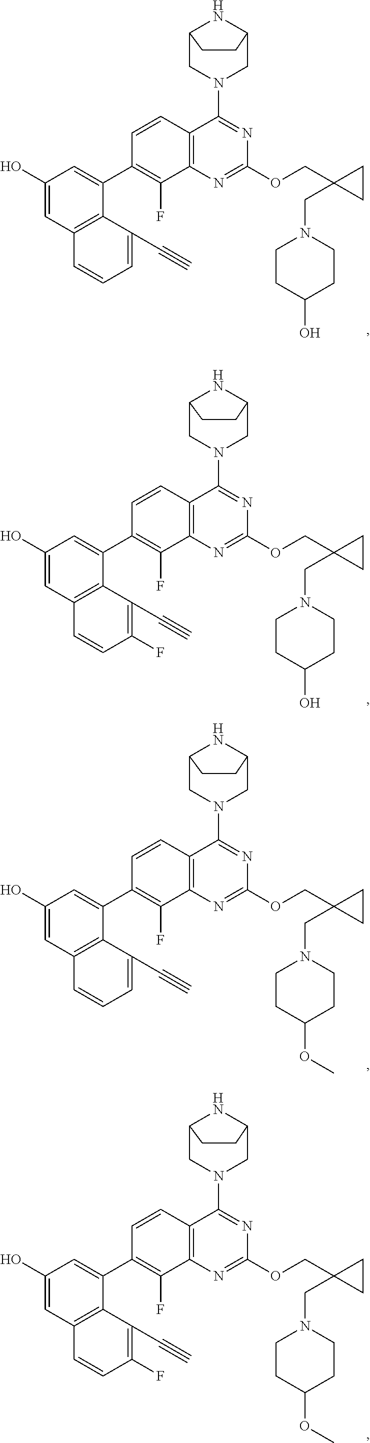 [SMILES: C#Cc1cccc2cc(O)cc(-c3ccc4c(N5CC6CCC(C5)N6)nc(OCC5(CN6CCC(O)CC6)CC5)nc4c3F)c12 ; C#Cc1c(F)ccc2cc(O)cc(-c3ccc4c(N5CC6CCC(C5)N6)nc(OCC5(CN6CCC(O)CC6)CC5)nc4c3F)c12 ; C#Cc1cccc2cc(O)cc(-c3ccc4c(N5CC6CCC(C5)N6)nc(OCC5(CN6CCC(OC)CC6)CC5)nc4c3F)c12 ; C#Cc1c(F)ccc2cc(O)cc(-c3ccc4c(N5CC6CCC(C5)N6)nc(OCC5(CN6CCC(OC)CC6)CC5)nc4c3F)c12]

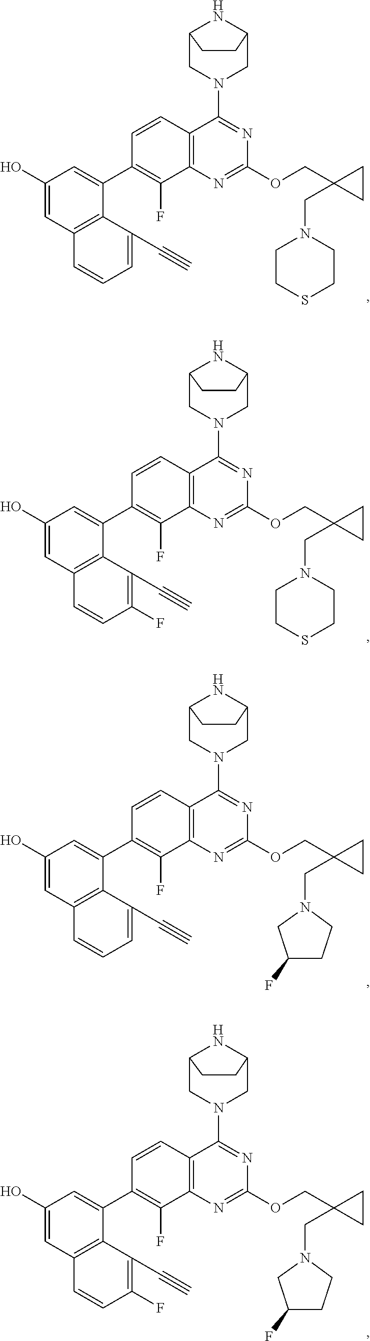 [SMILES: C#Cc1cccc2cc(O)cc(-c3ccc4c(N5CC6CCC(C5)N6)nc(OCC5(CN6CCSCC6)CC5)nc4c3F)c12 ; C#Cc1c(F)ccc2cc(O)cc(-c3ccc4c(N5CC6CCC(C5)N6)nc(OCC5(CN6CCSCC6)CC5)nc4c3F)c12 ; C#Cc1cccc2cc(O)cc(-c3ccc4c(N5CC6CCC(C5)N6)nc(OCC5(CN6CC[C@@H](F)C6)CC5)nc4c3F)c12 ; C#Cc1c(F)ccc2cc(O)cc(-c3ccc4c(N5CC6CCC(C5)N6)nc(OCC5(CN6CC[C@@H](F)C6)CC5)nc4c3F)c12]

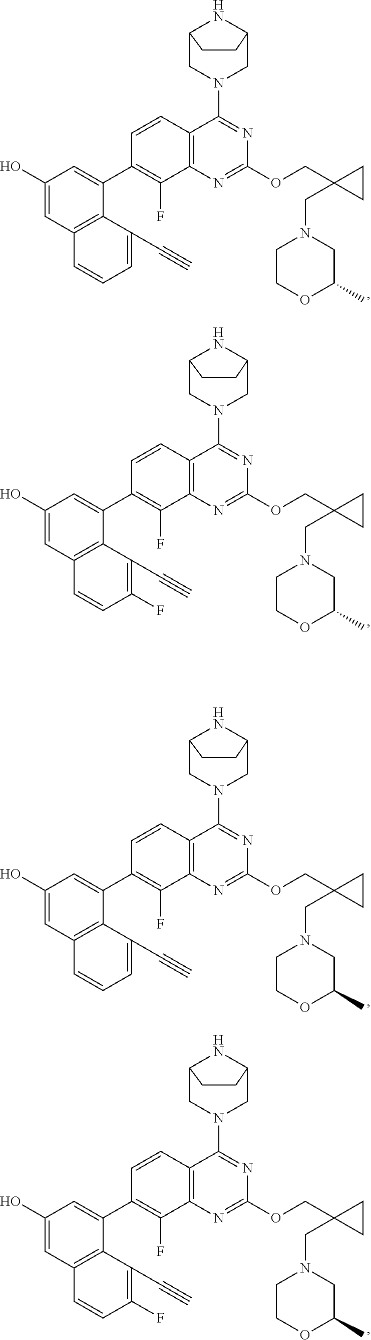 [SMILES: C#Cc1cccc2cc(O)cc(-c3ccc4c(N5CC6CCC(C5)N6)nc(OCC5(CN6CCO[C@@H](C)C6)CC5)nc4c3F)c12 ; C#Cc1c(F)ccc2cc(O)cc(-c3ccc4c(N5CC6CCC(C5)N6)nc(OCC5(CN6CCO[C@@H](C)C6)CC5)nc4c3F)c12 ; C#Cc1cccc2cc(O)cc(-c3ccc4c(N5CC6CCC(C5)N6)nc(OCC5(CN6CCO[C@H](C)C6)CC5)nc4c3F)c12 ; C#Cc1c(F)ccc2cc(O)cc(-c3ccc4c(N5CC6CCC(C5)N6)nc(OCC5(CN6CCO[C@H](C)C6)CC5)nc4c3F)c12]

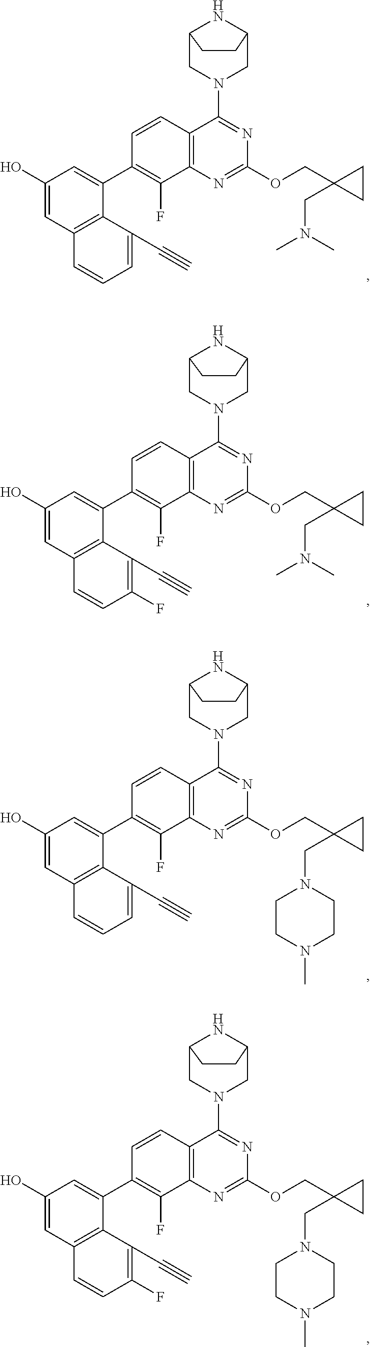 [SMILES: C#Cc1cccc2cc(O)cc(-c3ccc4c(N5CC6CCC(C5)N6)nc(OCC5(CN(C)C)CC5)nc4c3F)c12 ; C#Cc1c(F)ccc2cc(O)cc(-c3ccc4c(N5CC6CCC(C5)N6)nc(OCC5(CN(C)C)CC5)nc4c3F)c12 ; C#Cc1cccc2cc(O)cc(-c3ccc4c(N5CC6CCC(C5)N6)nc(OCC5(CN6CCN(C)CC6)CC5)nc4c3F)c12 ; C#Cc1c(F)ccc2cc(O)cc(-c3ccc4c(N5CC6CCC(C5)N6)nc(OCC5(CN6CCN(C)CC6)CC5)nc4c3F)c12]

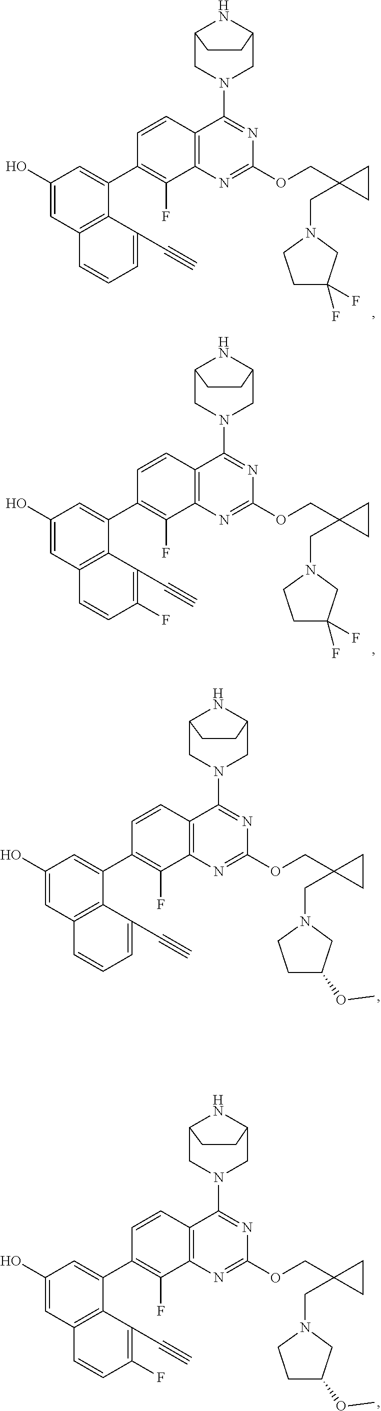 [SMILES: C#Cc1cccc2cc(O)cc(-c3ccc4c(N5CC6CCC(C5)N6)nc(OCC5(CN6CCC(F)(F)C6)CC5)nc4c3F)c12 ; C#Cc1c(F)ccc2cc(O)cc(-c3ccc4c(N5CC6CCC(C5)N6)nc(OCC5(CN6CCC(F)(F)C6)CC5)nc4c3F)c12 ; C#Cc1cccc2cc(O)cc(-c3ccc4c(N5CC6CCC(C5)N6)nc(OCC5(CN6CC[C@@H](OC)C6)CC5)nc4c3F)c12 ; C#Cc1c(F)ccc2cc(O)cc(-c3ccc4c(N5CC6CCC(C5)N6)nc(OCC5(CN6CC[C@@H](OC)C6)CC5)nc4c3F)c12]

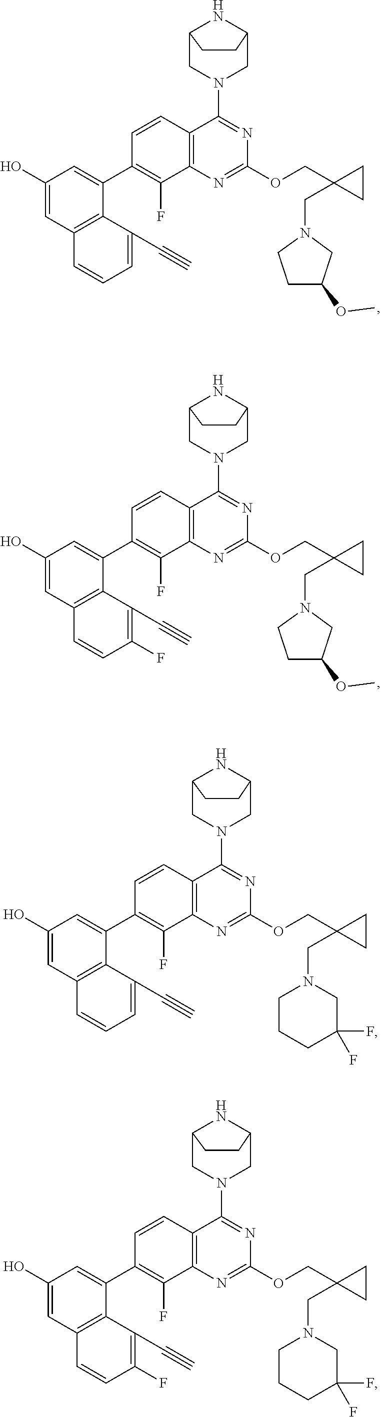 [SMILES: C#Cc1cccc2cc(O)cc(-c3ccc4c(N5CC6CCC(C5)N6)nc(OCC5(CN6CC[C@H](OC)C6)CC5)nc4c3F)c12 ; C#Cc1c(F)ccc2cc(O)cc(-c3ccc4c(N5CC6CCC(C5)N6)nc(OCC5(CN6CC[C@H](OC)C6)CC5)nc4c3F)c12 ; C#Cc1cccc2cc(O)cc(-c3ccc4c(N5CC6CCC(C5)N6)nc(OCC5(CN6CCCC(F)(F)C6)CC5)nc4c3F)c12 ; C#Cc1c(F)ccc2cc(O)cc(-c3ccc4c(N5CC6CCC(C5)N6)nc(OCC5(CN6CCCC(F)(F)C6)CC5)nc4c3F)c12]

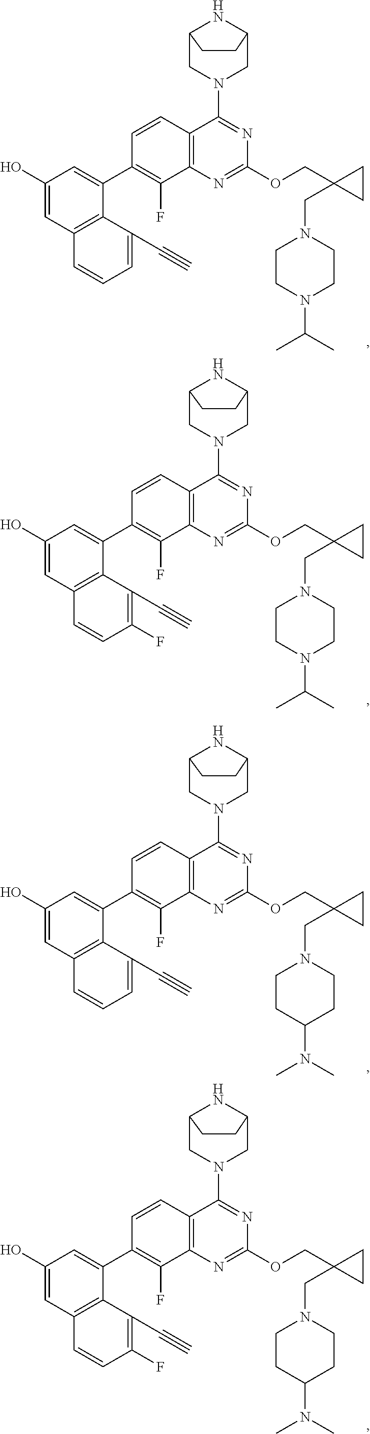 [SMILES: C#Cc1cccc2cc(O)cc(-c3ccc4c(N5CC6CCC(C5)N6)nc(OCC5(CN6CCN(C(C)C)CC6)CC5)nc4c3F)c12 ; C#Cc1c(F)ccc2cc(O)cc(-c3ccc4c(N5CC6CCC(C5)N6)nc(OCC5(CN6CCN(C(C)C)CC6)CC5)nc4c3F)c12 ; C#Cc1cccc2cc(O)cc(-c3ccc4c(N5CC6CCC(C5)N6)nc(OCC5(CN6CCC(N(C)C)CC6)CC5)nc4c3F)c12 ; C#Cc1c(F)ccc2cc(O)cc(-c3ccc4c(N5CC6CCC(C5)N6)nc(OCC5(CN6CCC(N(C)C)CC6)CC5)nc4c3F)c12]

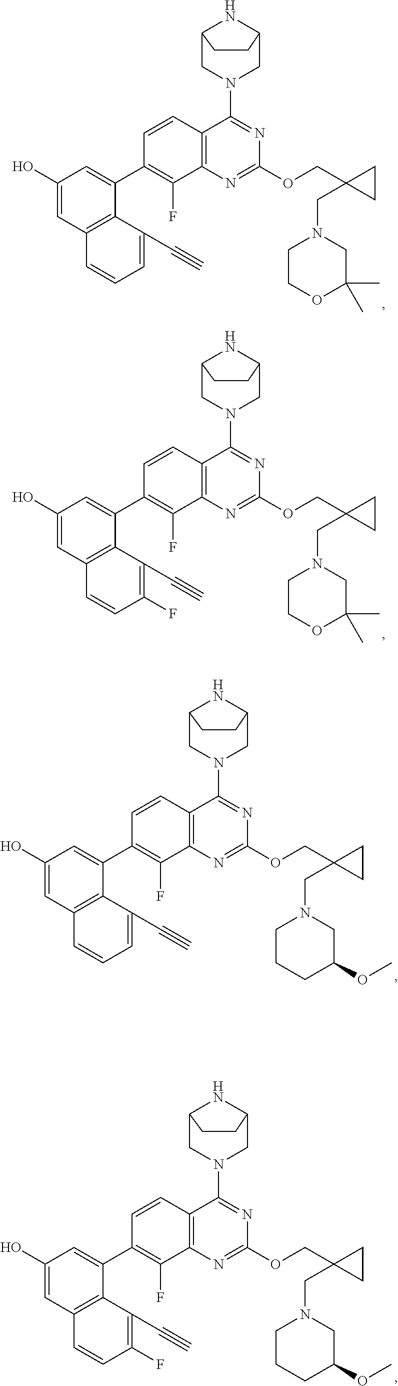 [SMILES: C#Cc1cccc2cc(O)cc(-c3ccc4c(N5CC6CCC(C5)N6)nc(OCC5(CN6CCOC(C)(C)C6)CC5)nc4c3F)c12 ; C#Cc1c(F)ccc2cc(O)cc(-c3ccc4c(N5CC6CCC(C5)N6)nc(OCC5(CN6CCOC(C)(C)C6)CC5)nc4c3F)c12 ; C#Cc1cccc2cc(O)cc(-c3ccc4c(N5CC6CCC(C5)N6)nc(OCC5(CN6CCC[C@H](OC)C6)CC5)nc4c3F)c12 ; C#Cc1c(F)ccc2cc(O)cc(-c3ccc4c(N5CC6CCC(C5)N6)nc(OCC5(CN6CCC[C@H](OC)C6)CC5)nc4c3F)c12]

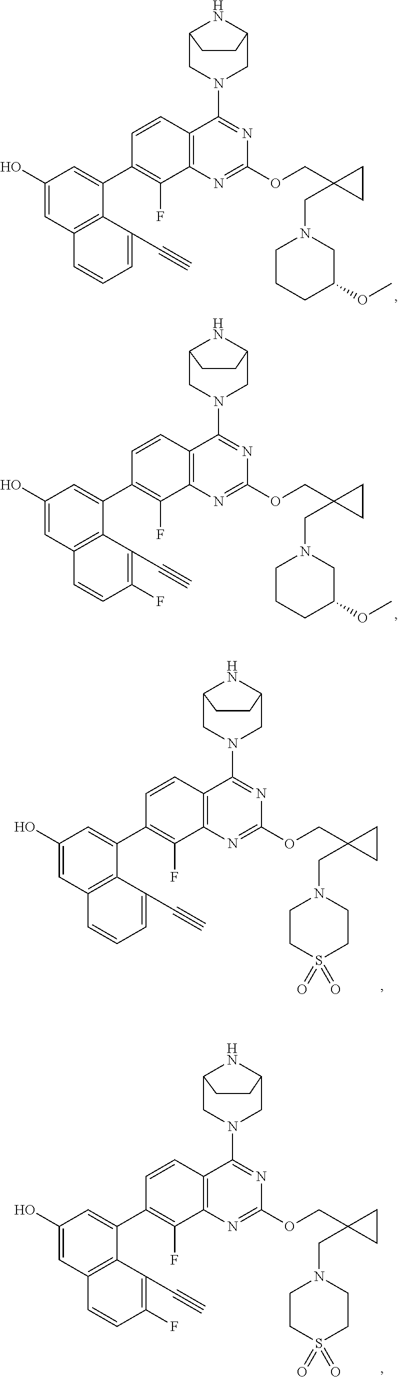 [SMILES: C#Cc1cccc2cc(O)cc(-c3ccc4c(N5CC6CCC(C5)N6)nc(OCC5(CN6CCC[C@@H](OC)C6)CC5)nc4c3F)c12 ; C#Cc1c(F)ccc2cc(O)cc(-c3ccc4c(N5CC6CCC(C5)N6)nc(OCC5(CN6CCC[C@@H](OC)C6)CC5)nc4c3F)c12 ; C#Cc1cccc2cc(O)cc(-c3ccc4c(N5CC6CCC(C5)N6)nc(OCC5(CN6CCS(=O)(=O)CC6)CC5)nc4c3F)c12 ; C#Cc1c(F)ccc2cc(O)cc(-c3ccc4c(N5CC6CCC(C5)N6)nc(OCC5(CN6CCS(=O)(=O)CC6)CC5)nc4c3F)c12]

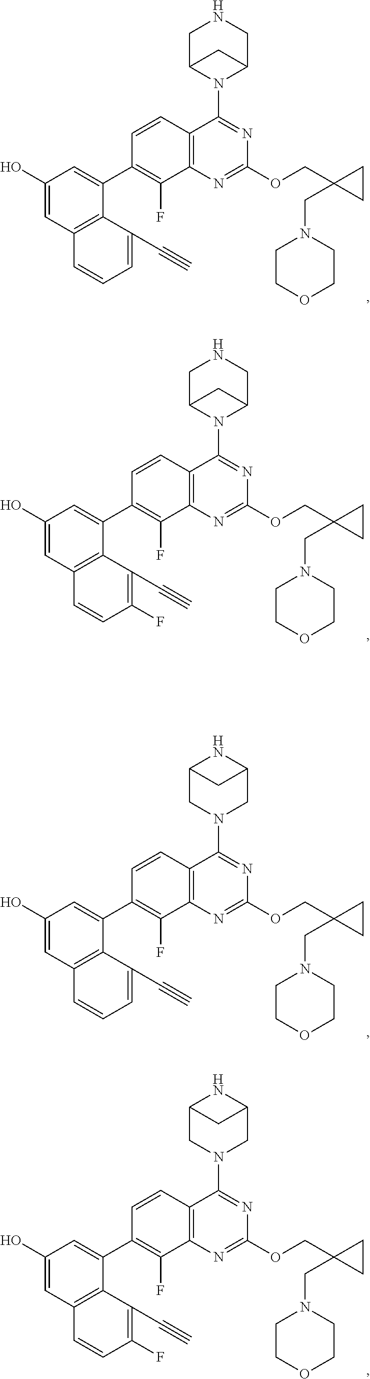 [SMILES: C#Cc1cccc2cc(O)cc(-c3ccc4c(N5C6CNCC5C6)nc(OCC5(CN6CCOCC6)CC5)nc4c3F)c12 ; C#Cc1c(F)ccc2cc(O)cc(-c3ccc4c(N5C6CNCC5C6)nc(OCC5(CN6CCOCC6)CC5)nc4c3F)c12 ; C#Cc1cccc2cc(O)cc(-c3ccc4c(N5CC6CC(C5)N6)nc(OCC5(CN6CCOCC6)CC5)nc4c3F)c12 ; C#Cc1c(F)ccc2cc(O)cc(-c3ccc4c(N5CC6CC(C5)N6)nc(OCC5(CN6CCOCC6)CC5)nc4c3F)c12]

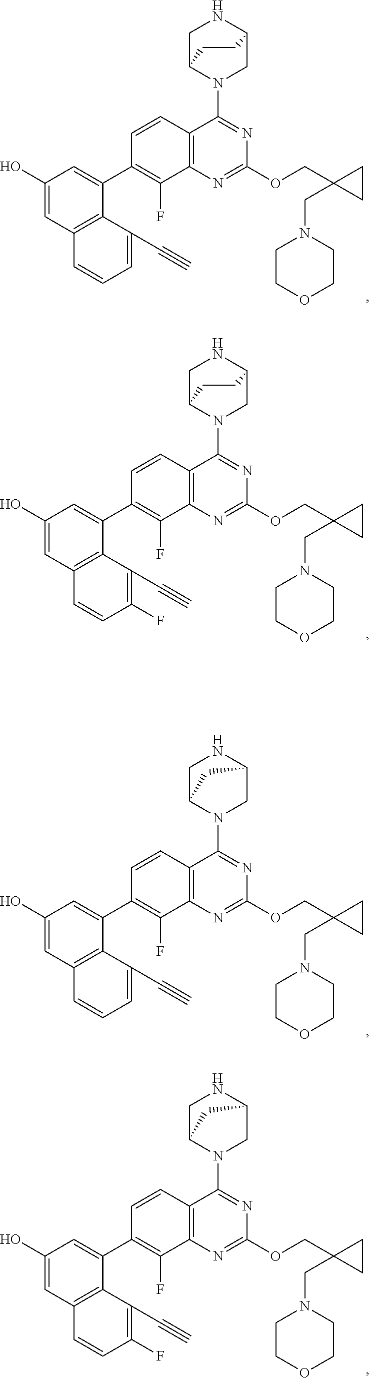 [SMILES: C#Cc1cccc2cc(O)cc(-c3ccc4c(N5C[C@H]6CC[C@@H]5CN6)nc(OCC5(CN6CCOCC6)CC5)nc4c3F)c12 ; C#Cc1c(F)ccc2cc(O)cc(-c3ccc4c(N5C[C@H]6CC[C@@H]5CN6)nc(OCC5(CN6CCOCC6)CC5)nc4c3F)c12 ; C#Cc1cccc2cc(O)cc(-c3ccc4c(N5C[C@H]6C[C@@H]5CN6)nc(OCC5(CN6CCOCC6)CC5)nc4c3F)c12 ; C#Cc1c(F)ccc2cc(O)cc(-c3ccc4c(N5C[C@H]6C[C@@H]5CN6)nc(OCC5(CN6CCOCC6)CC5)nc4c3F)c12]

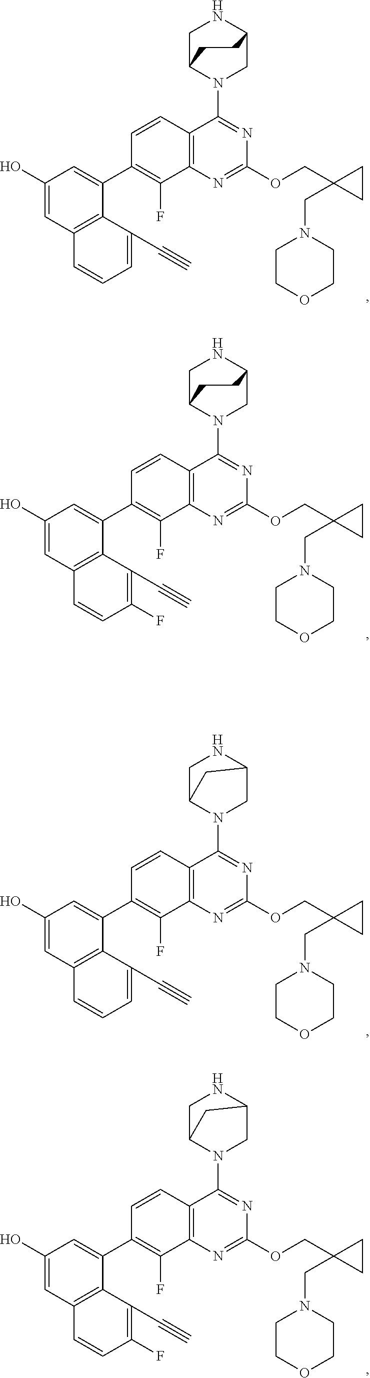 [SMILES: C#Cc1cccc2cc(O)cc(-c3ccc4c(N5C[C@@H]6CC[C@H]5CN6)nc(OCC5(CN6CCOCC6)CC5)nc4c3F)c12 ; C#Cc1c(F)ccc2cc(O)cc(-c3ccc4c(N5C[C@@H]6CC[C@H]5CN6)nc(OCC5(CN6CCOCC6)CC5)nc4c3F)c12 ; C#Cc1cccc2cc(O)cc(-c3ccc4c(N5CC6CC5CN6)nc(OCC5(CN6CCOCC6)CC5)nc4c3F)c12 ; C#Cc1c(F)ccc2cc(O)cc(-c3ccc4c(N5CC6CC5CN6)nc(OCC5(CN6CCOCC6)CC5)nc4c3F)c12]

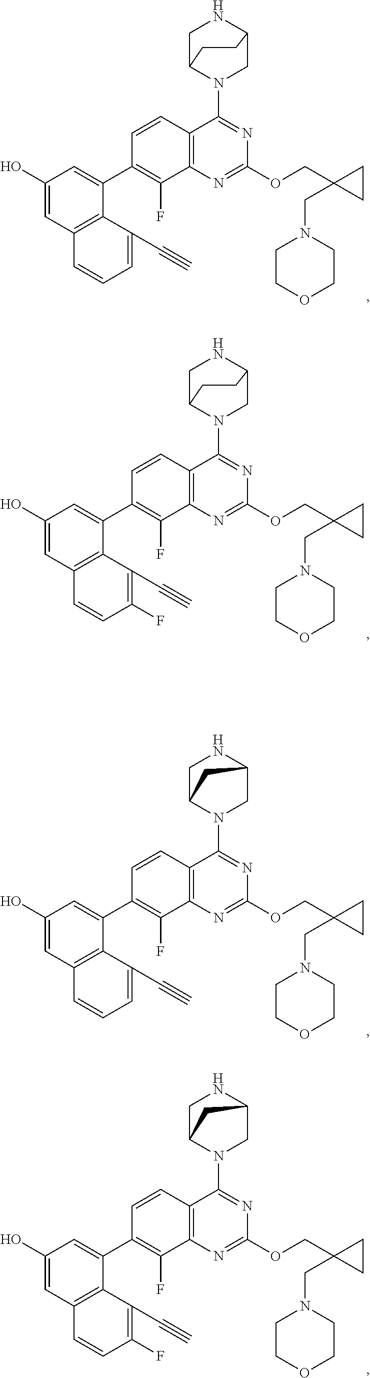 [SMILES: C#Cc1cccc2cc(O)cc(-c3ccc4c(N5CC6CCC5CN6)nc(OCC5(CN6CCOCC6)CC5)nc4c3F)c12 ; C#Cc1c(F)ccc2cc(O)cc(-c3ccc4c(N5CC6CCC5CN6)nc(OCC5(CN6CCOCC6)CC5)nc4c3F)c12 ; C#Cc1cccc2cc(O)cc(-c3ccc4c(N5C[C@@H]6C[C@H]5CN6)nc(OCC5(CN6CCOCC6)CC5)nc4c3F)c12 ; C#Cc1c(F)ccc2cc(O)cc(-c3ccc4c(N5C[C@@H]6C[C@H]5CN6)nc(OCC5(CN6CCOCC6)CC5)nc4c3F)c12]

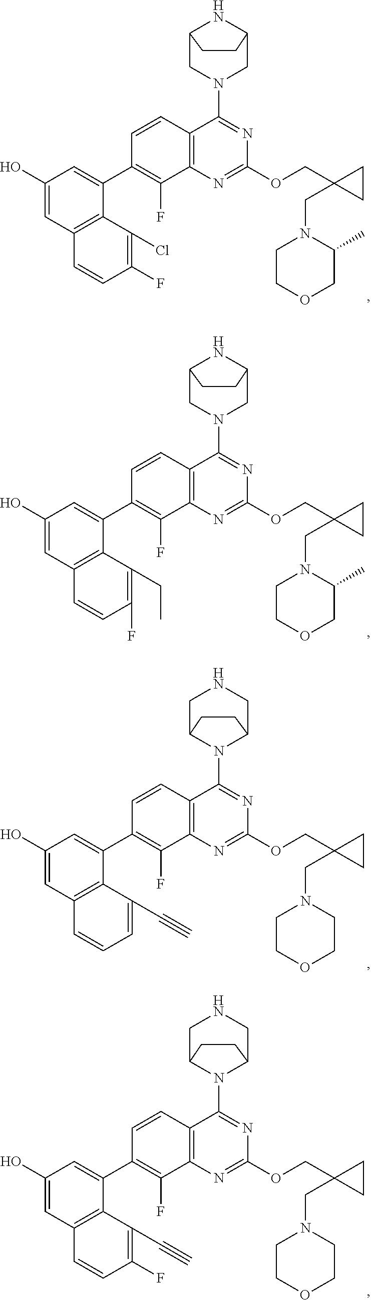 [SMILES: C[C@@H]1COCCN1CC1(COc2nc(N3CC4CCC(C3)N4)c3ccc(-c4cc(O)cc5ccc(F)c(Cl)c45)c(F)c3n2)CC1 ; CCc1c(F)ccc2cc(O)cc(-c3ccc4c(N5CC6CCC(C5)N6)nc(OCC5(CN6CCOC[C@H]6C)CC5)nc4c3F)c12 ; C#Cc1cccc2cc(O)cc(-c3ccc4c(N5C6CCC5CNC6)nc(OCC5(CN6CCOCC6)CC5)nc4c3F)c12 ; C#Cc1c(F)ccc2cc(O)cc(-c3ccc4c(N5C6CCC5CNC6)nc(OCC5(CN6CCOCC6)CC5)nc4c3F)c12]

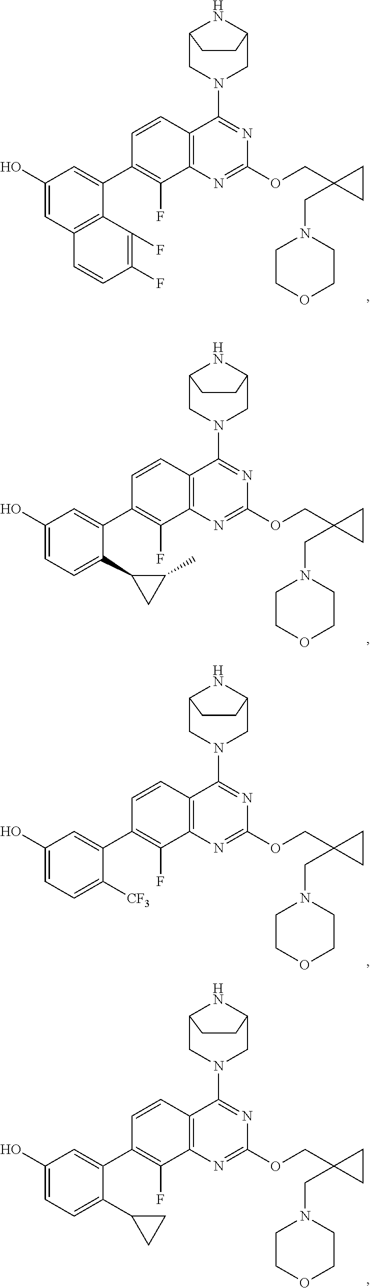 [SMILES: Oc1cc(-c2ccc3c(N4CC5CCC(C4)N5)nc(OCC4(CN5CCOCC5)CC4)nc3c2F)c2c(F)c(F)ccc2c1 ; C[C@@H]1C[C@H]1c1ccc(O)cc1-c1ccc2c(N3CC4CCC(C3)N4)nc(OCC3(CN4CCOCC4)CC3)nc2c1F ; Oc1ccc(C(F)(F)F)c(-c2ccc3c(N4CC5CCC(C4)N5)nc(OCC4(CN5CCOCC5)CC4)nc3c2F)c1 ; Oc1ccc(C2CC2)c(-c2ccc3c(N4CC5CCC(C4)N5)nc(OCC4(CN5CCOCC5)CC4)nc3c2F)c1]

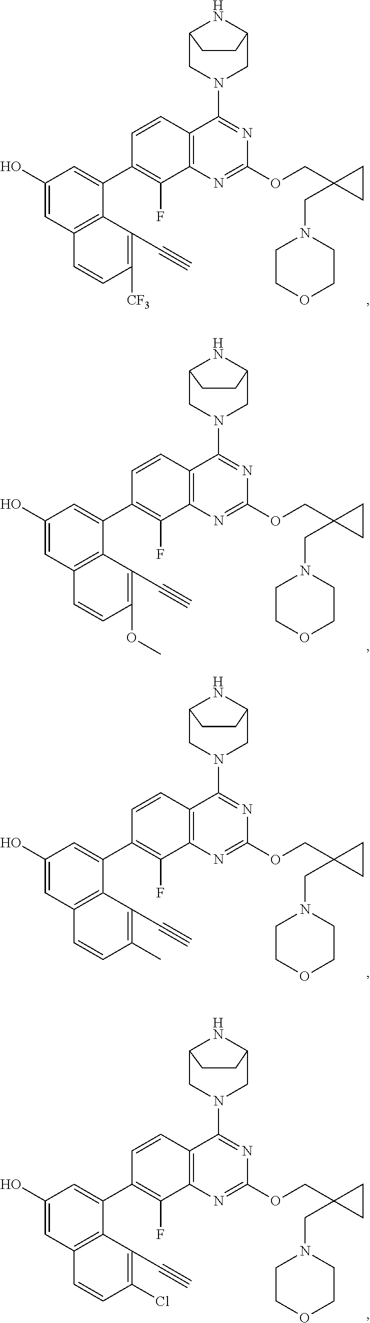 [SMILES: C#Cc1c(C(F)(F)F)ccc2cc(O)cc(-c3ccc4c(N5CC6CCC(C5)N6)nc(OCC5(CN6CCOCC6)CC5)nc4c3F)c12 ; C#Cc1c(OC)ccc2cc(O)cc(-c3ccc4c(N5CC6CCC(C5)N6)nc(OCC5(CN6CCOCC6)CC5)nc4c3F)c12 ; C#Cc1c(C)ccc2cc(O)cc(-c3ccc4c(N5CC6CCC(C5)N6)nc(OCC5(CN6CCOCC6)CC5)nc4c3F)c12 ; C#Cc1c(Cl)ccc2cc(O)cc(-c3ccc4c(N5CC6CCC(C5)N6)nc(OCC5(CN6CCOCC6)CC5)nc4c3F)c12]

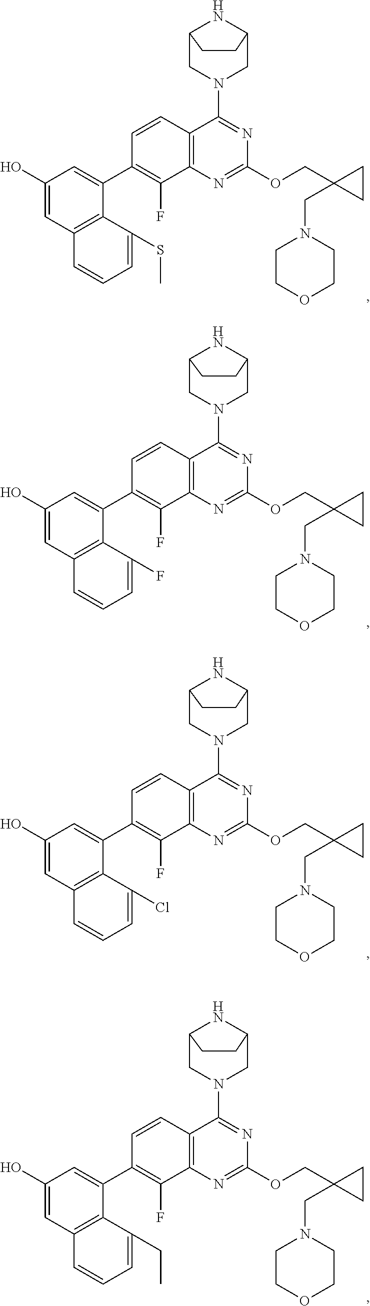 [SMILES: CSc1cccc2cc(O)cc(-c3ccc4c(N5CC6CCC(C5)N6)nc(OCC5(CN6CCOCC6)CC5)nc4c3F)c12 ; Oc1cc(-c2ccc3c(N4CC5CCC(C4)N5)nc(OCC4(CN5CCOCC5)CC4)nc3c2F)c2c(F)cccc2c1 ; Oc1cc(-c2ccc3c(N4CC5CCC(C4)N5)nc(OCC4(CN5CCOCC5)CC4)nc3c2F)c2c(Cl)cccc2c1 ; CCc1cccc2cc(O)cc(-c3ccc4c(N5CC6CCC(C5)N6)nc(OCC5(CN6CCOCC6)CC5)nc4c3F)c12]

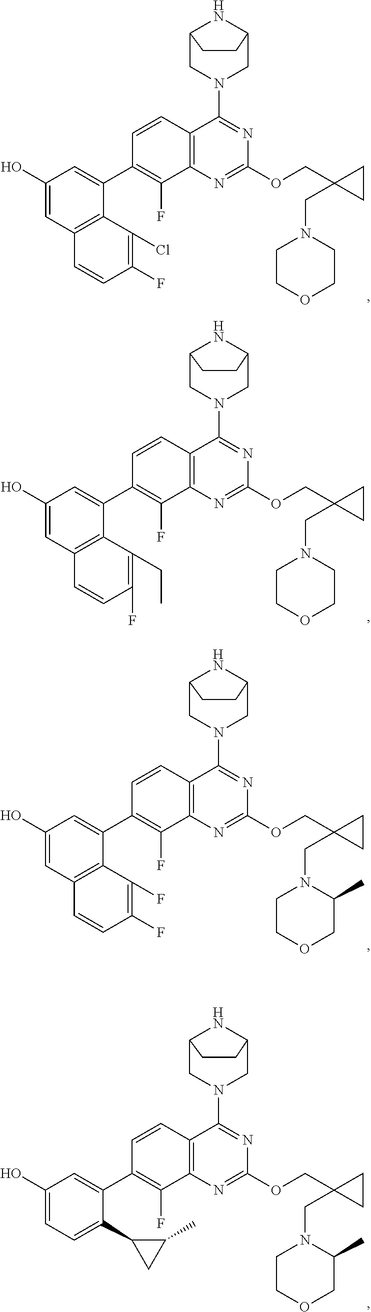 [SMILES: Oc1cc(-c2ccc3c(N4CC5CCC(C4)N5)nc(OCC4(CN5CCOCC5)CC4)nc3c2F)c2c(Cl)c(F)ccc2c1 ; CCc1c(F)ccc2cc(O)cc(-c3ccc4c(N5CC6CCC(C5)N6)nc(OCC5(CN6CCOCC6)CC5)nc4c3F)c12 ; C[C@H]1COCCN1CC1(COc2nc(N3CC4CCC(C3)N4)c3ccc(-c4cc(O)cc5ccc(F)c(F)c45)c(F)c3n2)CC1 ; C[C@@H]1C[C@H]1c1ccc(O)cc1-c1ccc2c(N3CC4CCC(C3)N4)nc(OCC3(CN4CCOC[C@@H]4C)CC3)nc2c1F]

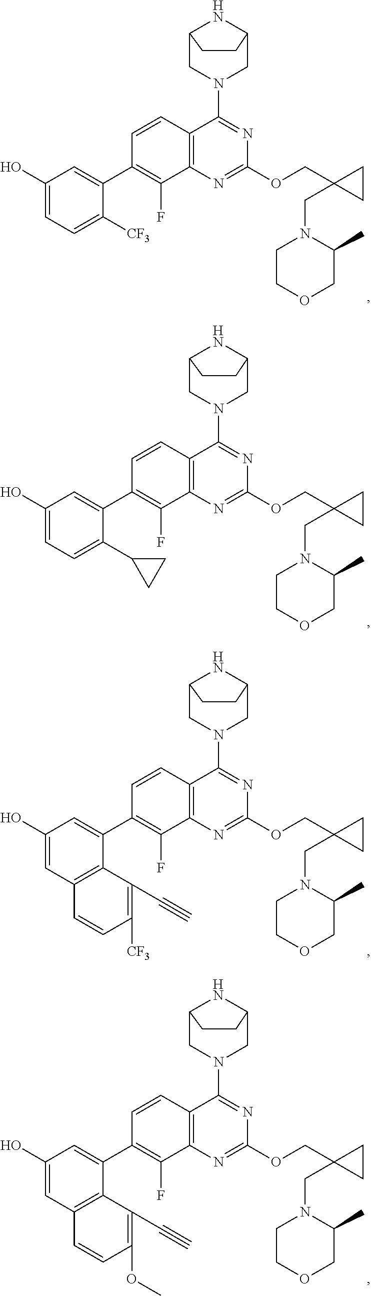 [SMILES: C[C@H]1COCCN1CC1(COc2nc(N3CC4CCC(C3)N4)c3ccc(-c4cc(O)ccc4C(F)(F)F)c(F)c3n2)CC1 ; C[C@H]1COCCN1CC1(COc2nc(N3CC4CCC(C3)N4)c3ccc(-c4cc(O)ccc4C4CC4)c(F)c3n2)CC1 ; C#Cc1c(C(F)(F)F)ccc2cc(O)cc(-c3ccc4c(N5CC6CCC(C5)N6)nc(OCC5(CN6CCOC[C@@H]6C)CC5)nc4c3F)c12 ; C#Cc1c(OC)ccc2cc(O)cc(-c3ccc4c(N5CC6CCC(C5)N6)nc(OCC5(CN6CCOC[C@@H]6C)CC5)nc4c3F)c12]

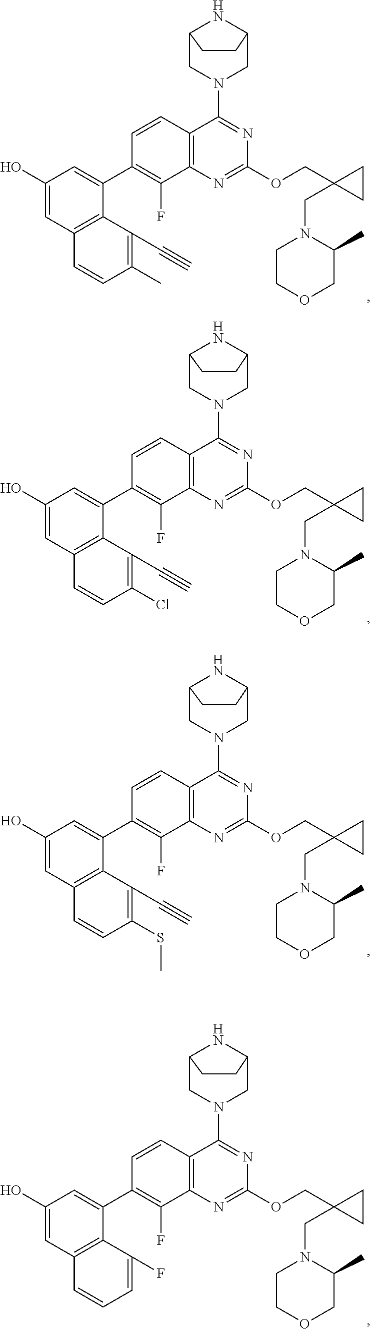 [SMILES: C#Cc1c(C)ccc2cc(O)cc(-c3ccc4c(N5CC6CCC(C5)N6)nc(OCC5(CN6CCOC[C@@H]6C)CC5)nc4c3F)c12 ; C#Cc1c(Cl)ccc2cc(O)cc(-c3ccc4c(N5CC6CCC(C5)N6)nc(OCC5(CN6CCOC[C@@H]6C)CC5)nc4c3F)c12 ; C#Cc1c(SC)ccc2cc(O)cc(-c3ccc4c(N5CC6CCC(C5)N6)nc(OCC5(CN6CCOC[C@@H]6C)CC5)nc4c3F)c12 ; C[C@H]1COCCN1CC1(COc2nc(N3CC4CCC(C3)N4)c3ccc(-c4cc(O)cc5cccc(F)c45)c(F)c3n2)CC1]

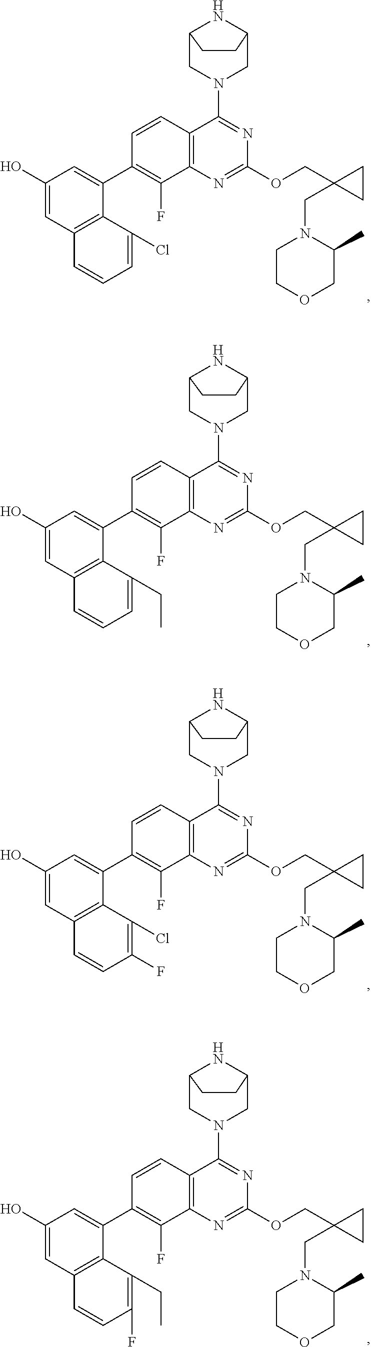 [SMILES: C[C@H]1COCCN1CC1(COc2nc(N3CC4CCC(C3)N4)c3ccc(-c4cc(O)cc5cccc(Cl)c45)c(F)c3n2)CC1 ; CCc1cccc2cc(O)cc(-c3ccc4c(N5CC6CCC(C5)N6)nc(OCC5(CN6CCOC[C@@H]6C)CC5)nc4c3F)c12 ; C[C@H]1COCCN1CC1(COc2nc(N3CC4CCC(C3)N4)c3ccc(-c4cc(O)cc5ccc(F)c(Cl)c45)c(F)c3n2)CC1 ; CCc1c(F)ccc2cc(O)cc(-c3ccc4c(N5CC6CCC(C5)N6)nc(OCC5(CN6CCOC[C@@H]6C)CC5)nc4c3F)c12]

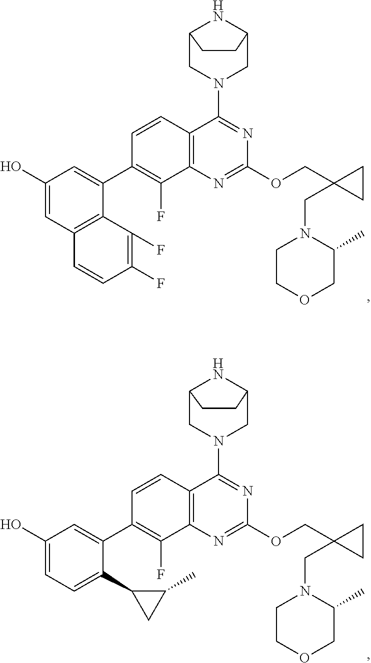 [SMILES: C[C@@H]1COCCN1CC1(COc2nc(N3CC4CCC(C3)N4)c3ccc(-c4cc(O)cc5ccc(F)c(F)c45)c(F)c3n2)CC1 ; C[C@@H]1C[C@H]1c1ccc(O)cc1-c1ccc2c(N3CC4CCC(C3)N4)nc(OCC3(CN4CCOC[C@H]4C)CC3)nc2c1F]

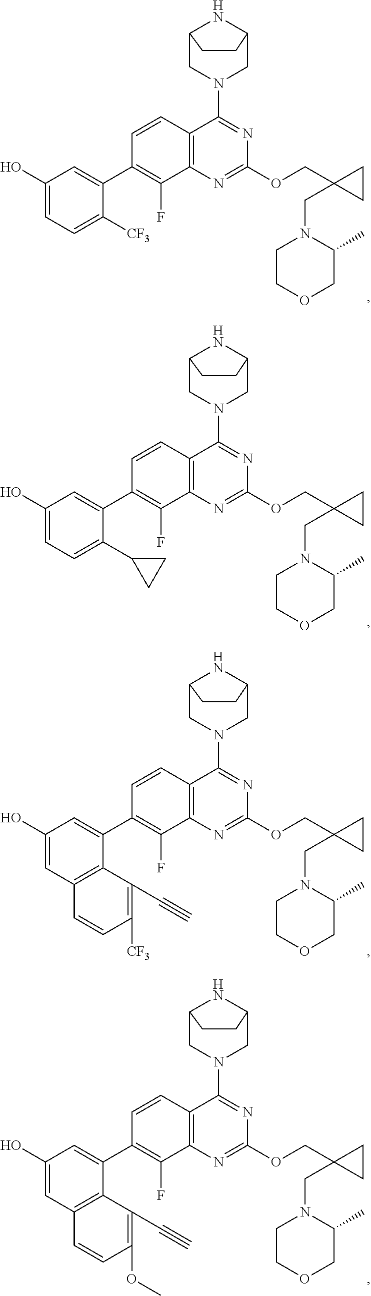 [SMILES: C[C@@H]1COCCN1CC1(COc2nc(N3CC4CCC(C3)N4)c3ccc(-c4cc(O)ccc4C(F)(F)F)c(F)c3n2)CC1 ; C[C@@H]1COCCN1CC1(COc2nc(N3CC4CCC(C3)N4)c3ccc(-c4cc(O)ccc4C4CC4)c(F)c3n2)CC1 ; C#Cc1c(C(F)(F)F)ccc2cc(O)cc(-c3ccc4c(N5CC6CCC(C5)N6)nc(OCC5(CN6CCOC[C@H]6C)CC5)nc4c3F)c12 ; C#Cc1c(OC)ccc2cc(O)cc(-c3ccc4c(N5CC6CCC(C5)N6)nc(OCC5(CN6CCOC[C@H]6C)CC5)nc4c3F)c12]

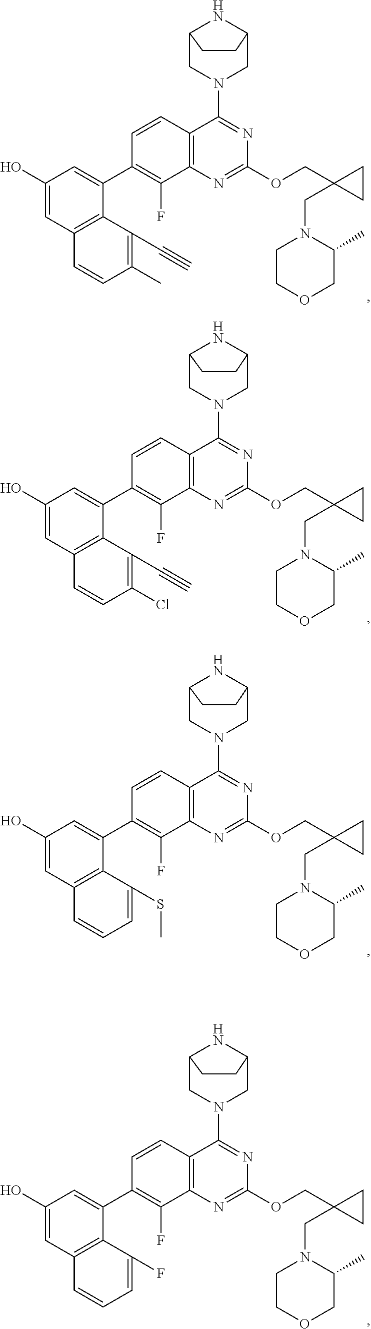 [SMILES: C#Cc1c(C)ccc2cc(O)cc(-c3ccc4c(N5CC6CCC(C5)N6)nc(OCC5(CN6CCOC[C@H]6C)CC5)nc4c3F)c12 ; C#Cc1c(Cl)ccc2cc(O)cc(-c3ccc4c(N5CC6CCC(C5)N6)nc(OCC5(CN6CCOC[C@H]6C)CC5)nc4c3F)c12 ; CSc1cccc2cc(O)cc(-c3ccc4c(N5CC6CCC(C5)N6)nc(OCC5(CN6CCOC[C@H]6C)CC5)nc4c3F)c12 ; C[C@@H]1COCCN1CC1(COc2nc(N3CC4CCC(C3)N4)c3ccc(-c4cc(O)cc5cccc(F)c45)c(F)c3n2)CC1]

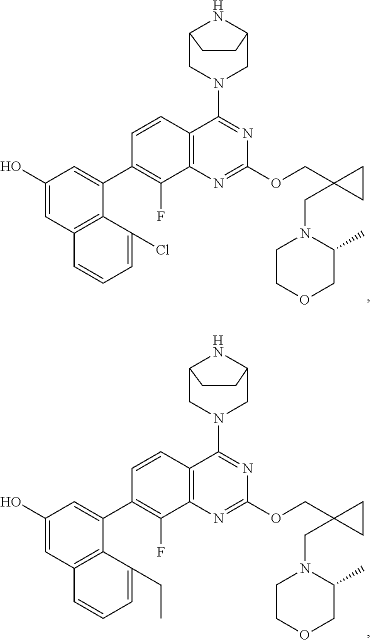 [SMILES: C[C@@H]1COCCN1CC1(COc2nc(N3CC4CCC(C3)N4)c3ccc(-c4cc(O)cc5cccc(Cl)c45)c(F)c3n2)CC1 ; CCc1cccc2cc(O)cc(-c3ccc4c(N5CC6CCC(C5)N6)nc(OCC5(CN6CCOC[C@H]6C)CC5)nc4c3F)c12]

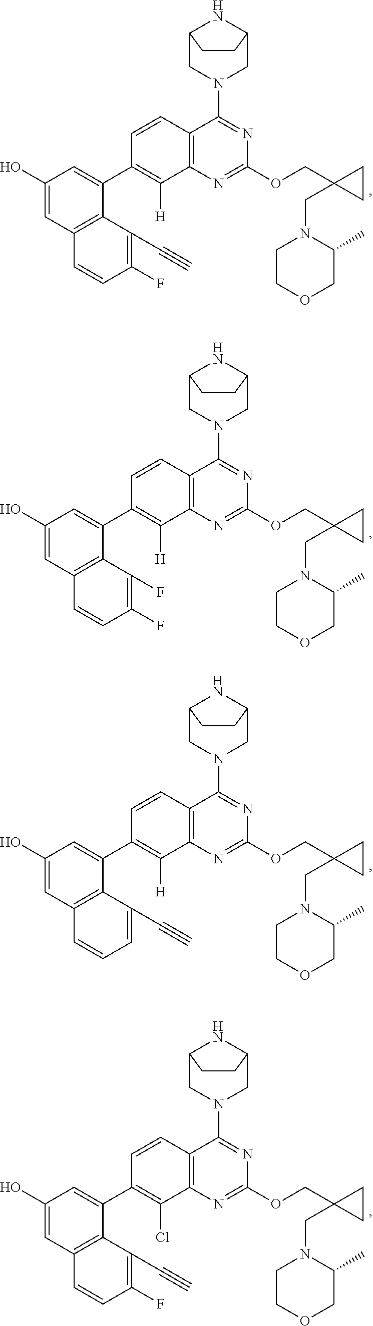 [SMILES: C#Cc1c(F)ccc2cc(O)cc(-c3ccc4c(N5CC6CCC(C5)N6)nc(OCC5(CN6CCOC[C@H]6C)CC5)nc4c3)c12 ; C[C@@H]1COCCN1CC1(COc2nc(N3CC4CCC(C3)N4)c3ccc(-c4cc(O)cc5ccc(F)c(F)c45)cc3n2)CC1 ; C#Cc1cccc2cc(O)cc(-c3ccc4c(N5CC6CCC(C5)N6)nc(OCC5(CN6CCOC[C@H]6C)CC5)nc4c3)c12 ; C#Cc1c(F)ccc2cc(O)cc(-c3ccc4c(N5CC6CCC(C5)N6)nc(OCC5(CN6CCOC[C@H]6C)CC5)nc4c3Cl)c12]

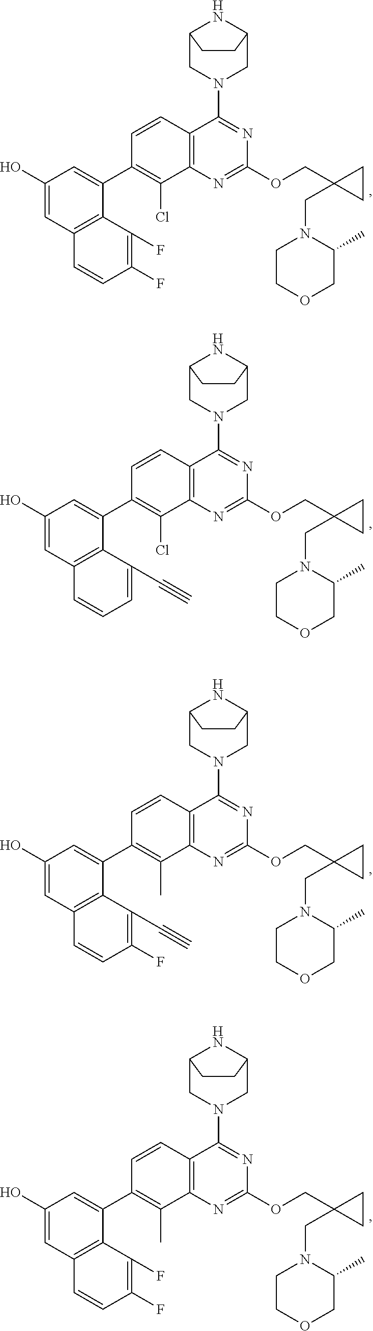 [SMILES: C[C@@H]1COCCN1CC1(COc2nc(N3CC4CCC(C3)N4)c3ccc(-c4cc(O)cc5ccc(F)c(F)c45)c(Cl)c3n2)CC1 ; C#Cc1cccc2cc(O)cc(-c3ccc4c(N5CC6CCC(C5)N6)nc(OCC5(CN6CCOC[C@H]6C)CC5)nc4c3Cl)c12 ; C#Cc1c(F)ccc2cc(O)cc(-c3ccc4c(N5CC6CCC(C5)N6)nc(OCC5(CN6CCOC[C@H]6C)CC5)nc4c3C)c12 ; Cc1c(-c2cc(O)cc3ccc(F)c(F)c23)ccc2c(N3CC4CCC(C3)N4)nc(OCC3(CN4CCOC[C@H]4C)CC3)nc12]

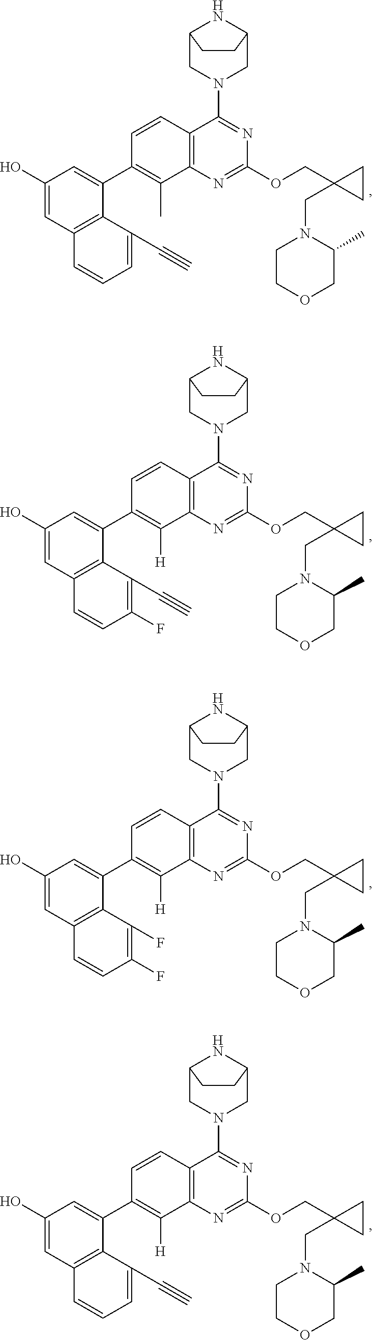 [SMILES: C#Cc1cccc2cc(O)cc(-c3ccc4c(N5CC6CCC(C5)N6)nc(OCC5(CN6CCOC[C@H]6C)CC5)nc4c3C)c12 ; C#Cc1c(F)ccc2cc(O)cc(-c3ccc4c(N5CC6CCC(C5)N6)nc(OCC5(CN6CCOC[C@@H]6C)CC5)nc4c3)c12 ; C[C@H]1COCCN1CC1(COc2nc(N3CC4CCC(C3)N4)c3ccc(-c4cc(O)cc5ccc(F)c(F)c45)cc3n2)CC1 ; C#Cc1cccc2cc(O)cc(-c3ccc4c(N5CC6CCC(C5)N6)nc(OCC5(CN6CCOC[C@@H]6C)CC5)nc4c3)c12]

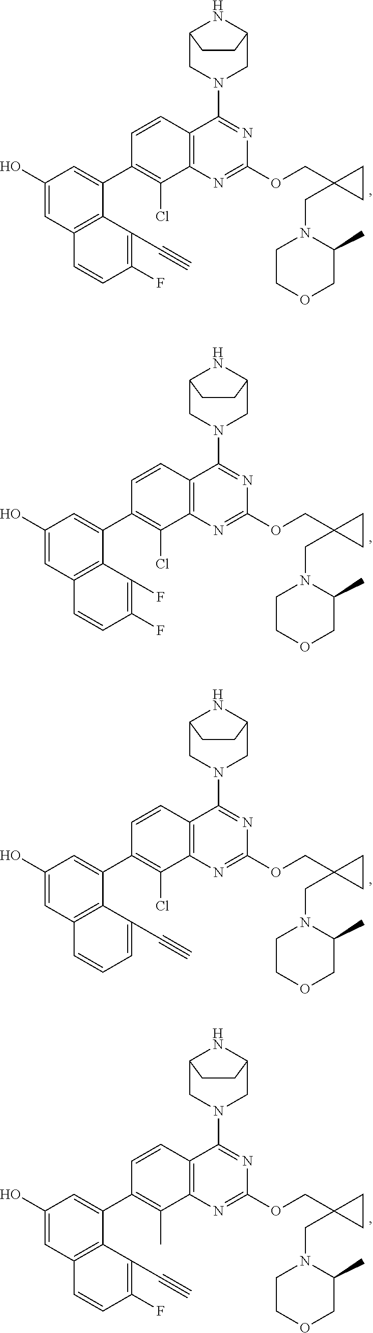 [SMILES: C#Cc1c(F)ccc2cc(O)cc(-c3ccc4c(N5CC6CCC(C5)N6)nc(OCC5(CN6CCOC[C@@H]6C)CC5)nc4c3Cl)c12 ; C[C@H]1COCCN1CC1(COc2nc(N3CC4CCC(C3)N4)c3ccc(-c4cc(O)cc5ccc(F)c(F)c45)c(Cl)c3n2)CC1 ; C#Cc1cccc2cc(O)cc(-c3ccc4c(N5CC6CCC(C5)N6)nc(OCC5(CN6CCOC[C@@H]6C)CC5)nc4c3Cl)c12 ; C#Cc1c(F)ccc2cc(O)cc(-c3ccc4c(N5CC6CCC(C5)N6)nc(OCC5(CN6CCOC[C@@H]6C)CC5)nc4c3C)c12]

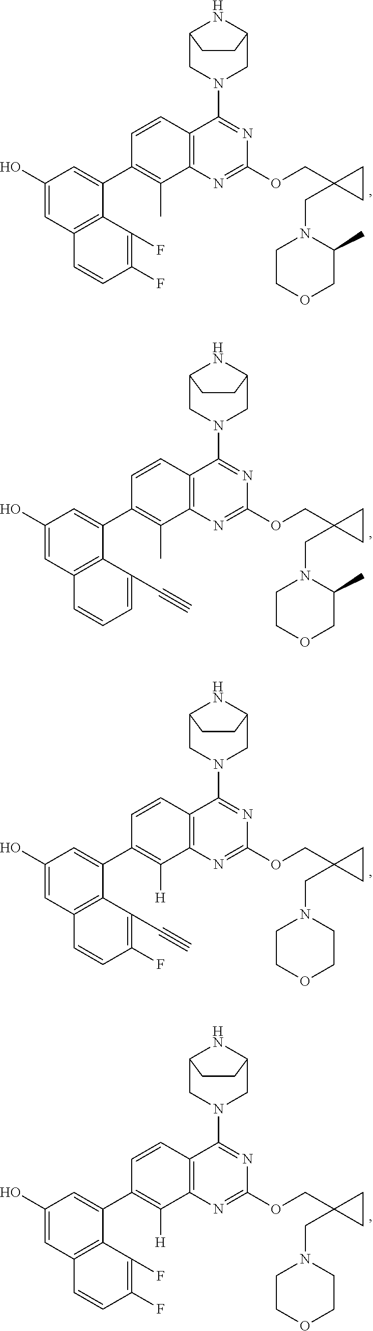 [SMILES: Cc1c(-c2cc(O)cc3ccc(F)c(F)c23)ccc2c(N3CC4CCC(C3)N4)nc(OCC3(CN4CCOC[C@@H]4C)CC3)nc12 ; C#Cc1cccc2cc(O)cc(-c3ccc4c(N5CC6CCC(C5)N6)nc(OCC5(CN6CCOC[C@@H]6C)CC5)nc4c3C)c12 ; C#Cc1c(F)ccc2cc(O)cc(-c3ccc4c(N5CC6CCC(C5)N6)nc(OCC5(CN6CCOCC6)CC5)nc4c3)c12 ; Oc1cc(-c2ccc3c(N4CC5CCC(C4)N5)nc(OCC4(CN5CCOCC5)CC4)nc3c2)c2c(F)c(F)ccc2c1]

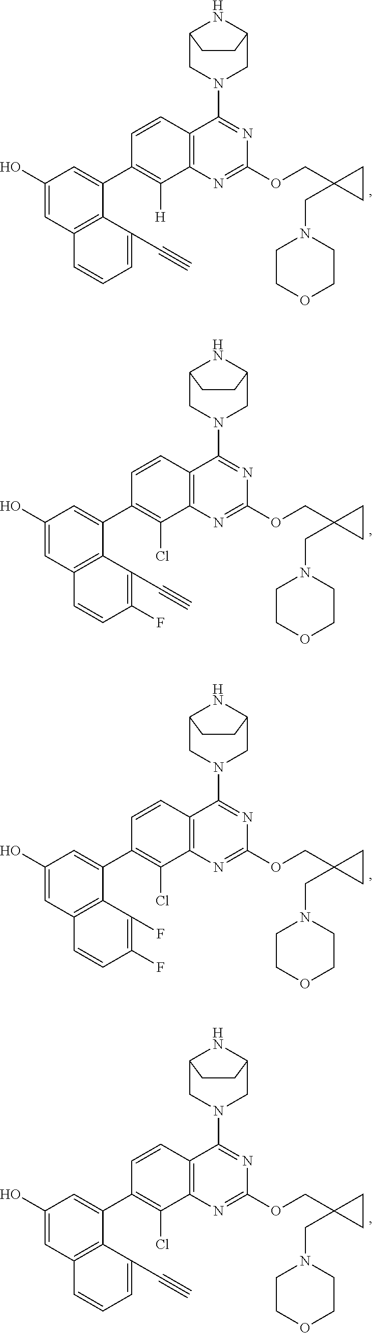 [SMILES: C#Cc1cccc2cc(O)cc(-c3ccc4c(N5CC6CCC(C5)N6)nc(OCC5(CN6CCOCC6)CC5)nc4c3)c12 ; C#Cc1c(F)ccc2cc(O)cc(-c3ccc4c(N5CC6CCC(C5)N6)nc(OCC5(CN6CCOCC6)CC5)nc4c3Cl)c12 ; Oc1cc(-c2ccc3c(N4CC5CCC(C4)N5)nc(OCC4(CN5CCOCC5)CC4)nc3c2Cl)c2c(F)c(F)ccc2c1 ; C#Cc1cccc2cc(O)cc(-c3ccc4c(N5CC6CCC(C5)N6)nc(OCC5(CN6CCOCC6)CC5)nc4c3Cl)c12]

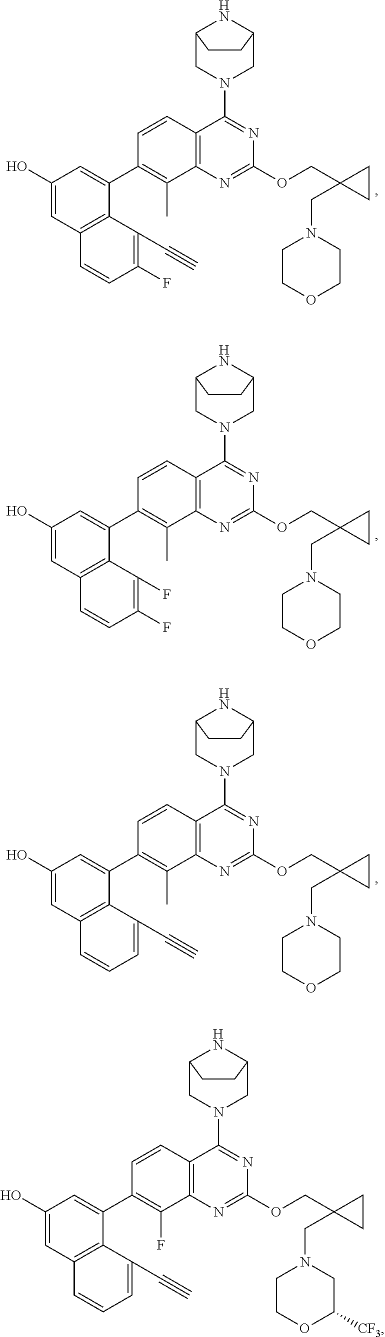 [SMILES: C#Cc1c(F)ccc2cc(O)cc(-c3ccc4c(N5CC6CCC(C5)N6)nc(OCC5(CN6CCOCC6)CC5)nc4c3C)c12 ; Cc1c(-c2cc(O)cc3ccc(F)c(F)c23)ccc2c(N3CC4CCC(C3)N4)nc(OCC3(CN4CCOCC4)CC3)nc12 ; C#Cc1cccc2cc(O)cc(-c3ccc4c(N5CC6CCC(C5)N6)nc(OCC5(CN6CCOCC6)CC5)nc4c3C)c12 ; C#Cc1cccc2cc(O)cc(-c3ccc4c(N5CC6CCC(C5)N6)nc(OCC5(CN6CCO[C@@H](C(F)(F)F)C6)CC5)nc4c3F)c12]

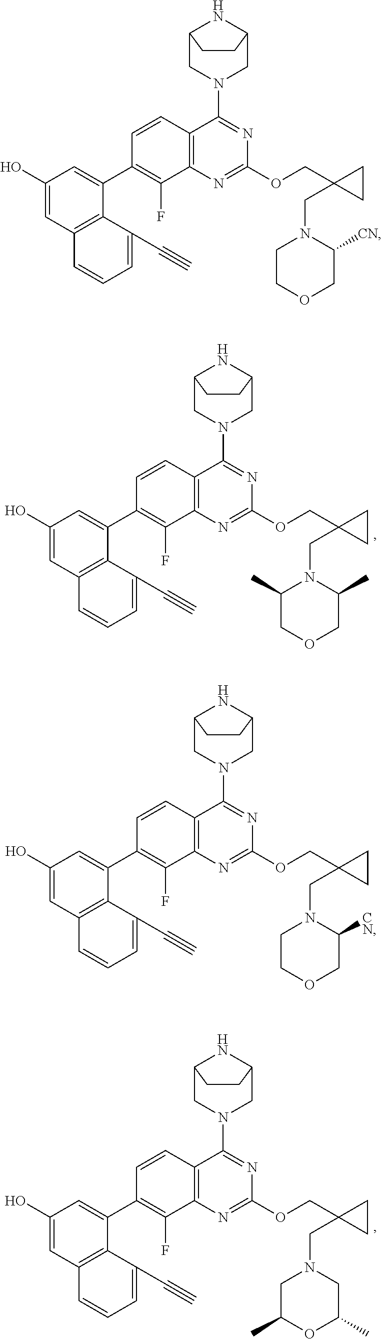 [SMILES: C#Cc1cccc2cc(O)cc(-c3ccc4c(N5CC6CCC(C5)N6)nc(OCC5(CN6CCOC[C@H]6C#N)CC5)nc4c3F)c12 ; C#Cc1cccc2cc(O)cc(-c3ccc4c(N5CC6CCC(C5)N6)nc(OCC5(CN6[C@H](C)COC[C@@H]6C)CC5)nc4c3F)c12 ; C#Cc1cccc2cc(O)cc(-c3ccc4c(N5CC6CCC(C5)N6)nc(OCC5(CN6CCOC[C@@H]6C#N)CC5)nc4c3F)c12 ; C#Cc1cccc2cc(O)cc(-c3ccc4c(N5CC6CCC(C5)N6)nc(OCC5(CN6C[C@H](C)O[C@@H](C)C6)CC5)nc4c3F)c12]

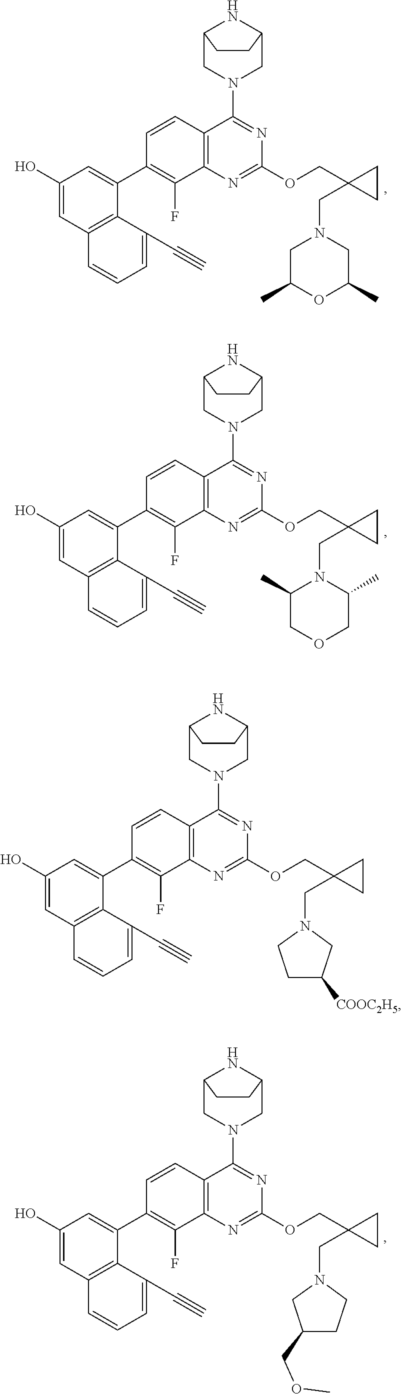

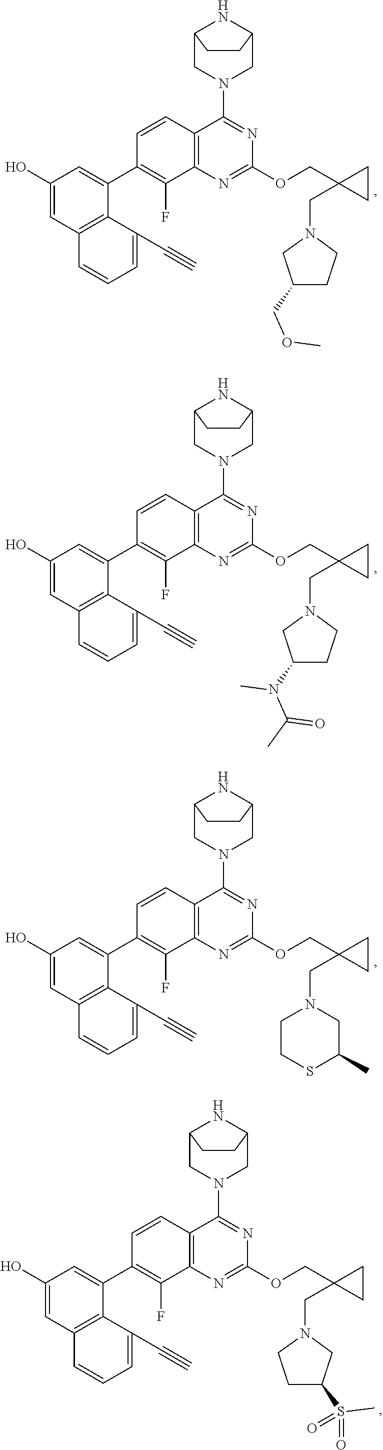 [SMILES: C#Cc1cccc2cc(O)cc(-c3ccc4c(N5CC6CCC(C5)N6)nc(OCC5(CN6CC[C@H](COC)C6)CC5)nc4c3F)c12 ; C#Cc1cccc2cc(O)cc(-c3ccc4c(N5CC6CCC(C5)N6)nc(OCC5(CN6CC[C@H](N(C)C(C)=O)C6)CC5)nc4c3F)c12 ; C#Cc1cccc2cc(O)cc(-c3ccc4c(N5CC6CCC(C5)N6)nc(OCC5(CN6CCS[C@H](C)C6)CC5)nc4c3F)c12 ; C#Cc1cccc2cc(O)cc(-c3ccc4c(N5CC6CCC(C5)N6)nc(OCC5(CN6CC[C@H](S(C)(=O)=O)C6)CC5)nc4c3F)c12]

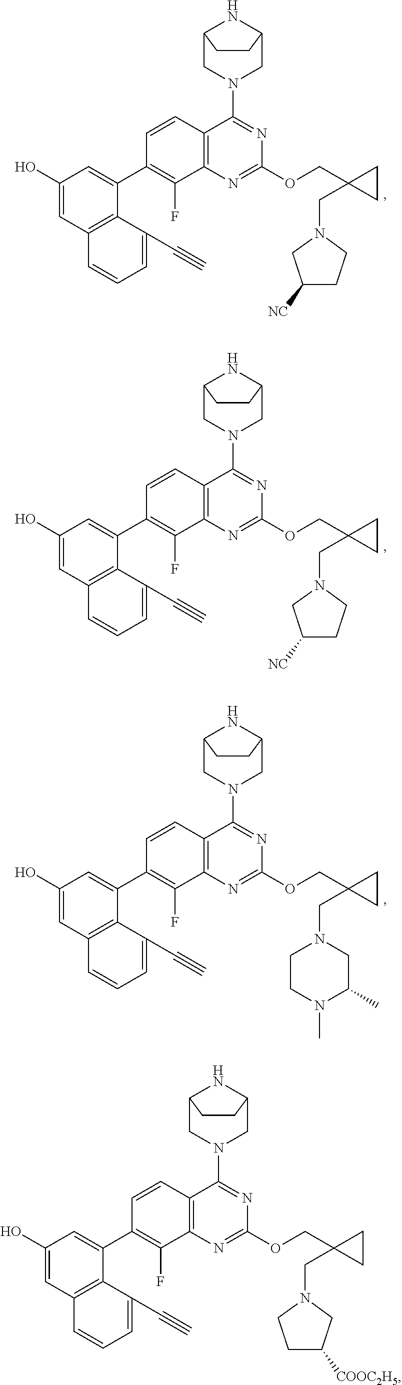

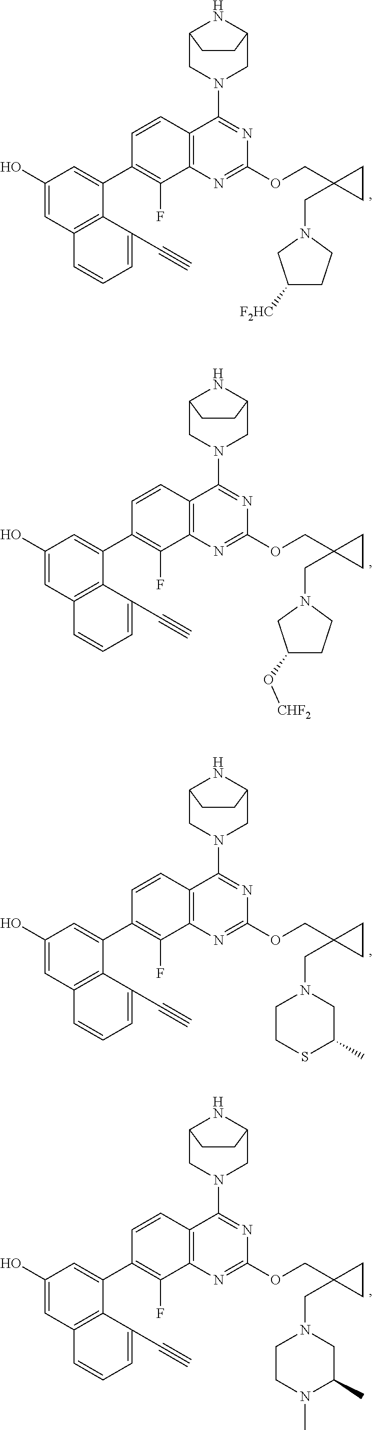 [SMILES: C#Cc1cccc2cc(O)cc(-c3ccc4c(N5CC6CCC(C5)N6)nc(OCC5(CN6CC[C@H](C(F)F)C6)CC5)nc4c3F)c12 ; C#Cc1cccc2cc(O)cc(-c3ccc4c(N5CC6CCC(C5)N6)nc(OCC5(CN6CC[C@H](OC(F)F)C6)CC5)nc4c3F)c12 ; C#Cc1cccc2cc(O)cc(-c3ccc4c(N5CC6CCC(C5)N6)nc(OCC5(CN6CCS[C@@H](C)C6)CC5)nc4c3F)c12 ; C#Cc1cccc2cc(O)cc(-c3ccc4c(N5CC6CCC(C5)N6)nc(OCC5(CN6CCN(C)[C@H](C)C6)CC5)nc4c3F)c12]

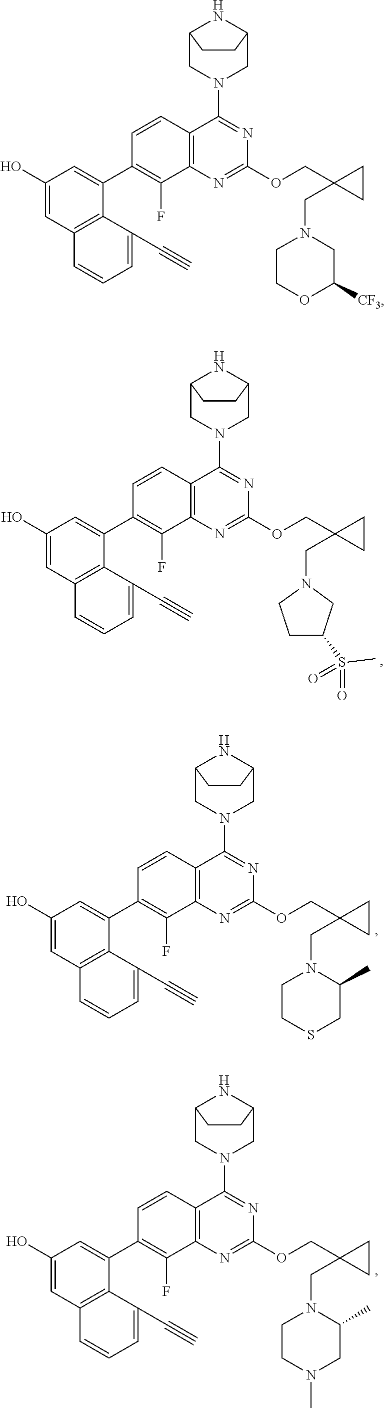 [SMILES: C#Cc1cccc2cc(O)cc(-c3ccc4c(N5CC6CCC(C5)N6)nc(OCC5(CN6CCO[C@H](C(F)(F)F)C6)CC5)nc4c3F)c12 ; C#Cc1cccc2cc(O)cc(-c3ccc4c(N5CC6CCC(C5)N6)nc(OCC5(CN6CC[C@@H](S(C)(=O)=O)C6)CC5)nc4c3F)c12 ; C#Cc1cccc2cc(O)cc(-c3ccc4c(N5CC6CCC(C5)N6)nc(OCC5(CN6CCSC[C@@H]6C)CC5)nc4c3F)c12 ; C#Cc1cccc2cc(O)cc(-c3ccc4c(N5CC6CCC(C5)N6)nc(OCC5(CN6CCN(C)C[C@H]6C)CC5)nc4c3F)c12]

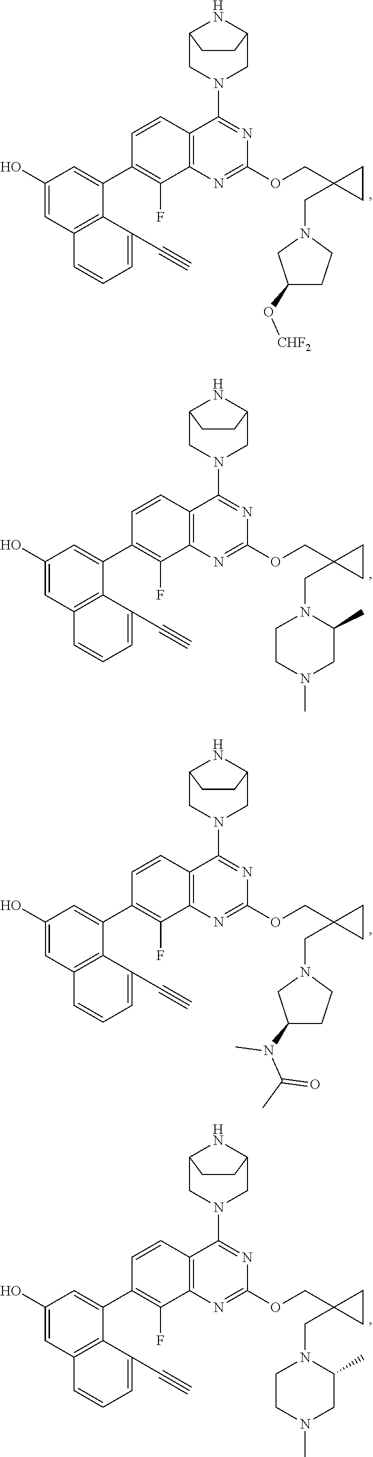 [SMILES: C#Cc1cccc2cc(O)cc(-c3ccc4c(N5CC6CCC(C5)N6)nc(OCC5(CN6CC[C@@H](OC(F)F)C6)CC5)nc4c3F)c12 ; C#Cc1cccc2cc(O)cc(-c3ccc4c(N5CC6CCC(C5)N6)nc(OCC5(CN6CCN(C)C[C@@H]6C)CC5)nc4c3F)c12 ; C#Cc1cccc2cc(O)cc(-c3ccc4c(N5CC6CCC(C5)N6)nc(OCC5(CN6CC[C@@H](N(C)C(C)=O)C6)CC5)nc4c3F)c12 ; C#Cc1cccc2cc(O)cc(-c3ccc4c(N5CC6CCC(C5)N6)nc(OCC5(CN6CCN(C)C[C@H]6C)CC5)nc4c3F)c12]

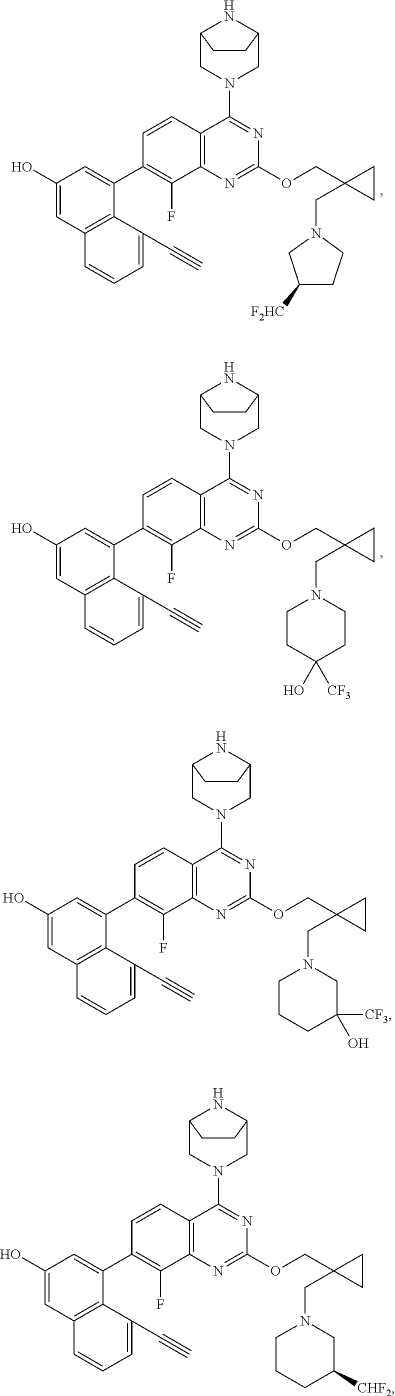 [SMILES: C#Cc1cccc2cc(O)cc(-c3ccc4c(N5CC6CCC(C5)N6)nc(OCC5(CN6CC[C@@H](C(F)F)C6)CC5)nc4c3F)c12 ; C#Cc1cccc2cc(O)cc(-c3ccc4c(N5CC6CCC(C5)N6)nc(OCC5(CN6CCC(O)(C(F)(F)F)CC6)CC5)nc4c3F)c12 ; C#Cc1cccc2cc(O)cc(-c3ccc4c(N5CC6CCC(C5)N6)nc(OCC5(CN6CCCC(O)(C(F)(F)F)C6)CC5)nc4c3F)c12 ; C#Cc1cccc2cc(O)cc(-c3ccc4c(N5CC6CCC(C5)N6)nc(OCC5(CN6CCC[C@H](C(F)F)C6)CC5)nc4c3F)c12]

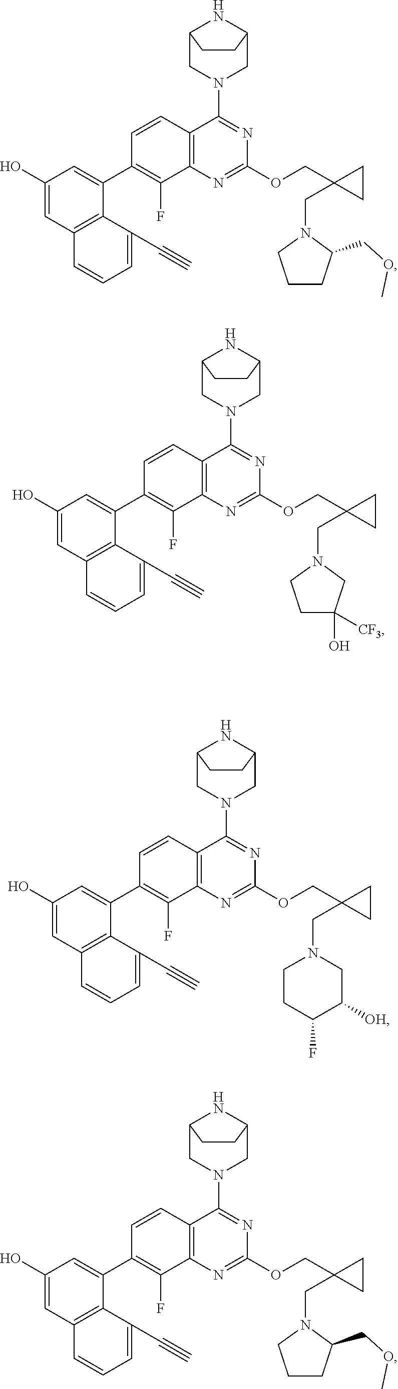 [SMILES: C#Cc1cccc2cc(O)cc(-c3ccc4c(N5CC6CCC(C5)N6)nc(OCC5(CN6CCC[C@H]6COC)CC5)nc4c3F)c12 ; C#Cc1cccc2cc(O)cc(-c3ccc4c(N5CC6CCC(C5)N6)nc(OCC5(CN6CCC(O)(C(F)(F)F)C6)CC5)nc4c3F)c12 ; C#Cc1cccc2cc(O)cc(-c3ccc4c(N5CC6CCC(C5)N6)nc(OCC5(CN6CC[C@@H](F)[C@@H](O)C6)CC5)nc4c3F)c12 ; C#Cc1cccc2cc(O)cc(-c3ccc4c(N5CC6CCC(C5)N6)nc(OCC5(CN6CCC[C@@H]6COC)CC5)nc4c3F)c12]

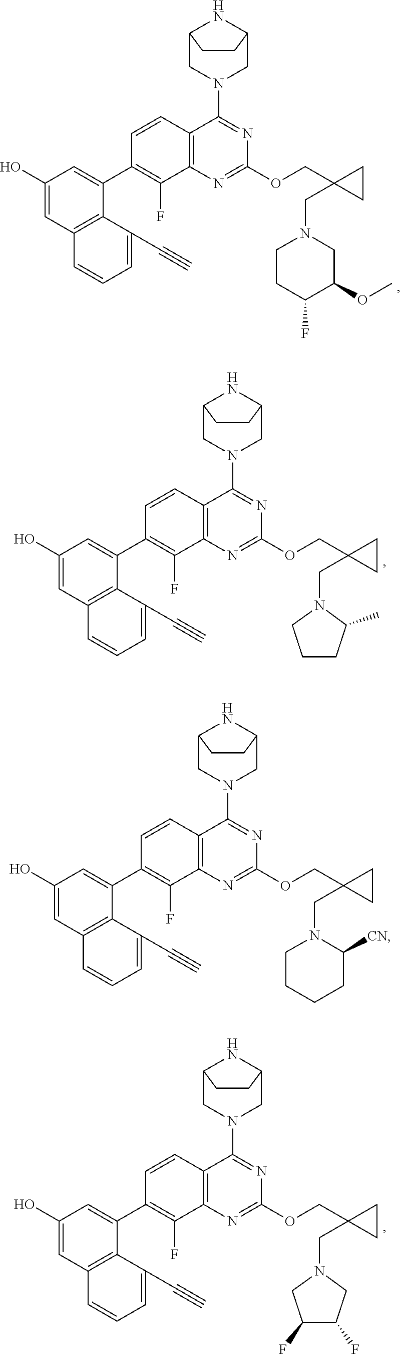 [SMILES: C#Cc1cccc2cc(O)cc(-c3ccc4c(N5CC6CCC(C5)N6)nc(OCC5(CN6CC[C@@H](F)[C@H](OC)C6)CC5)nc4c3F)c12 ; C#Cc1cccc2cc(O)cc(-c3ccc4c(N5CC6CCC(C5)N6)nc(OCC5(CN6CCC[C@H]6C)CC5)nc4c3F)c12 ; C#Cc1cccc2cc(O)cc(-c3ccc4c(N5CC6CCC(C5)N6)nc(OCC5(CN6CCCC[C@@H]6C#N)CC5)nc4c3F)c12 ; C#Cc1cccc2cc(O)cc(-c3ccc4c(N5CC6CCC(C5)N6)nc(OCC5(CN6C[C@H](F)[C@@H](F)C6)CC5)nc4c3F)c12]

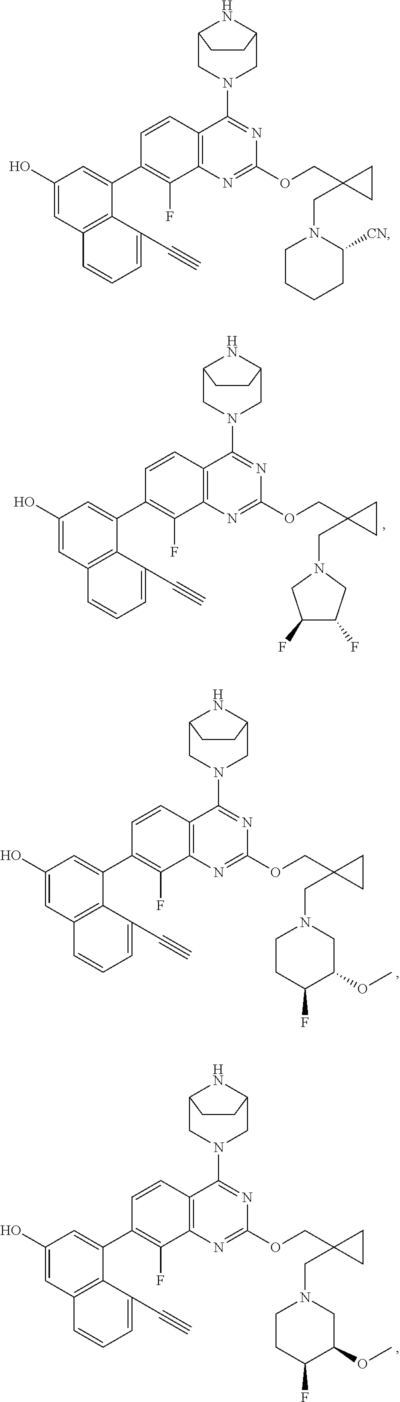 [SMILES: C#Cc1cccc2cc(O)cc(-c3ccc4c(N5CC6CCC(C5)N6)nc(OCC5(CN6CCCC[C@H]6C#N)CC5)nc4c3F)c12 ; C#Cc1cccc2cc(O)cc(-c3ccc4c(N5CC6CCC(C5)N6)nc(OCC5(CN6C[C@H](F)[C@@H](F)C6)CC5)nc4c3F)c12 ; C#Cc1cccc2cc(O)cc(-c3ccc4c(N5CC6CCC(C5)N6)nc(OCC5(CN6CC[C@H](F)[C@@H](OC)C6)CC5)nc4c3F)c12 ; C#Cc1cccc2cc(O)cc(-c3ccc4c(N5CC6CCC(C5)N6)nc(OCC5(CN6CC[C@H](F)[C@H](OC)C6)CC5)nc4c3F)c12]

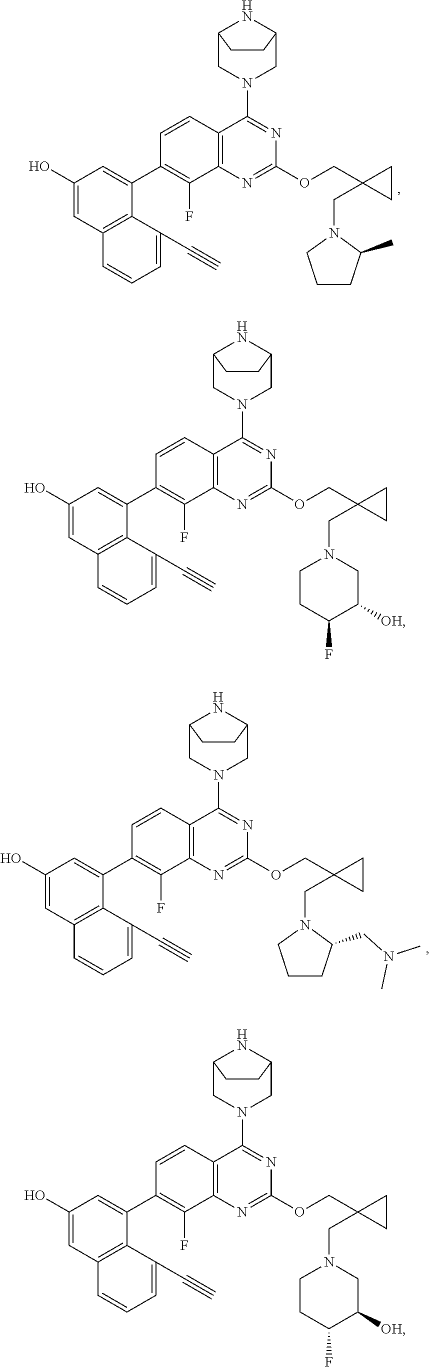 [SMILES: C#Cc1cccc2cc(O)cc(-c3ccc4c(N5CC6CCC(C5)N6)nc(OCC5(CN6CCC[C@@H]6C)CC5)nc4c3F)c12 ; C#Cc1cccc2cc(O)cc(-c3ccc4c(N5CC6CCC(C5)N6)nc(OCC5(CN6CC[C@H](F)[C@@H](O)C6)CC5)nc4c3F)c12 ; C#Cc1cccc2cc(O)cc(-c3ccc4c(N5CC6CCC(C5)N6)nc(OCC5(CN6CCC[C@H]6CN(C)C)CC5)nc4c3F)c12 ; C#Cc1cccc2cc(O)cc(-c3ccc4c(N5CC6CCC(C5)N6)nc(OCC5(CN6CC[C@@H](F)[C@H](O)C6)CC5)nc4c3F)c12]

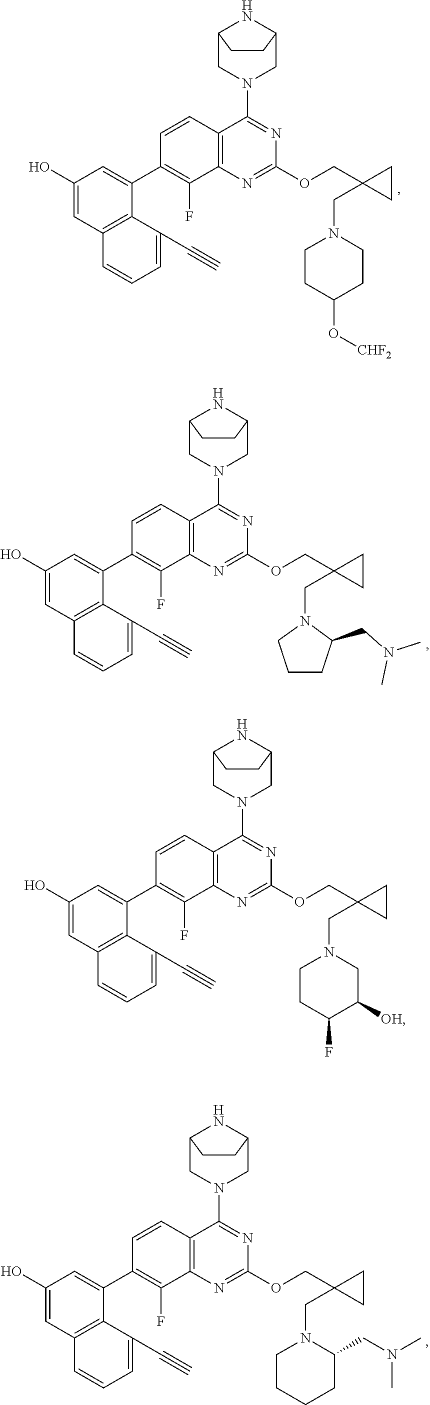 [SMILES: C#Cc1cccc2cc(O)cc(-c3ccc4c(N5CC6CCC(C5)N6)nc(OCC5(CN6CCC(OC(F)F)CC6)CC5)nc4c3F)c12 ; C#Cc1cccc2cc(O)cc(-c3ccc4c(N5CC6CCC(C5)N6)nc(OCC5(CN6CCC[C@@H]6CN(C)C)CC5)nc4c3F)c12 ; C#Cc1cccc2cc(O)cc(-c3ccc4c(N5CC6CCC(C5)N6)nc(OCC5(CN6CC[C@H](F)[C@H](O)C6)CC5)nc4c3F)c12 ; C#Cc1cccc2cc(O)cc(-c3ccc4c(N5CC6CCC(C5)N6)nc(OCC5(CN6CCCC[C@H]6CN(C)C)CC5)nc4c3F)c12]

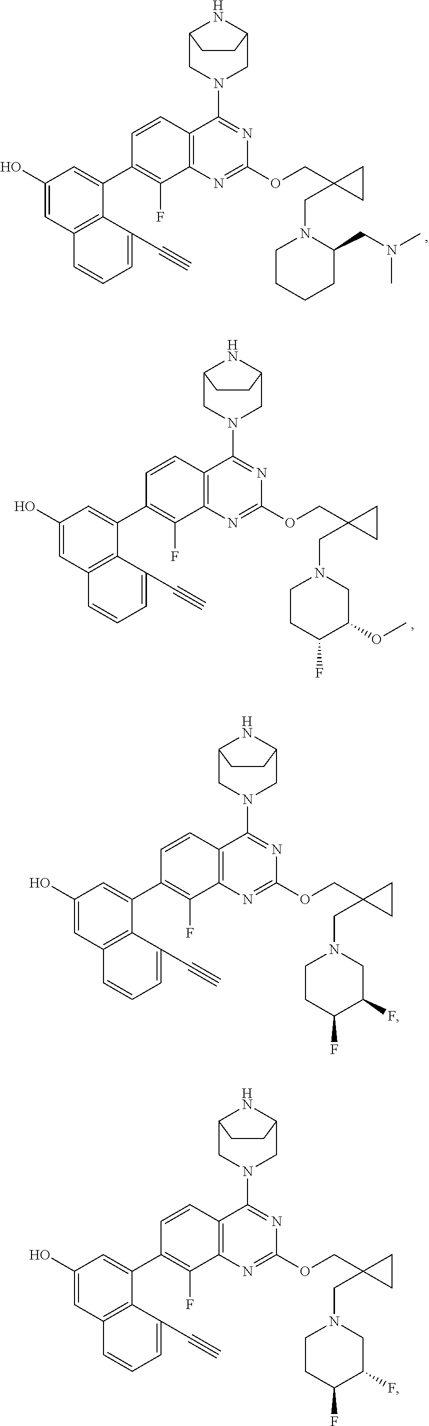 [SMILES: C#Cc1cccc2cc(O)cc(-c3ccc4c(N5CC6CCC(C5)N6)nc(OCC5(CN6CCCC[C@@H]6CN(C)C)CC5)nc4c3F)c12 ; C#Cc1cccc2cc(O)cc(-c3ccc4c(N5CC6CCC(C5)N6)nc(OCC5(CN6CC[C@@H](F)[C@@H](OC)C6)CC5)nc4c3F)c12 ; C#Cc1cccc2cc(O)cc(-c3ccc4c(N5CC6CCC(C5)N6)nc(OCC5(CN6CC[C@H](F)[C@H](F)C6)CC5)nc4c3F)c12 ; C#Cc1cccc2cc(O)cc(-c3ccc4c(N5CC6CCC(C5)N6)nc(OCC5(CN6CC[C@H](F)[C@@H](F)C6)CC5)nc4c3F)c12]

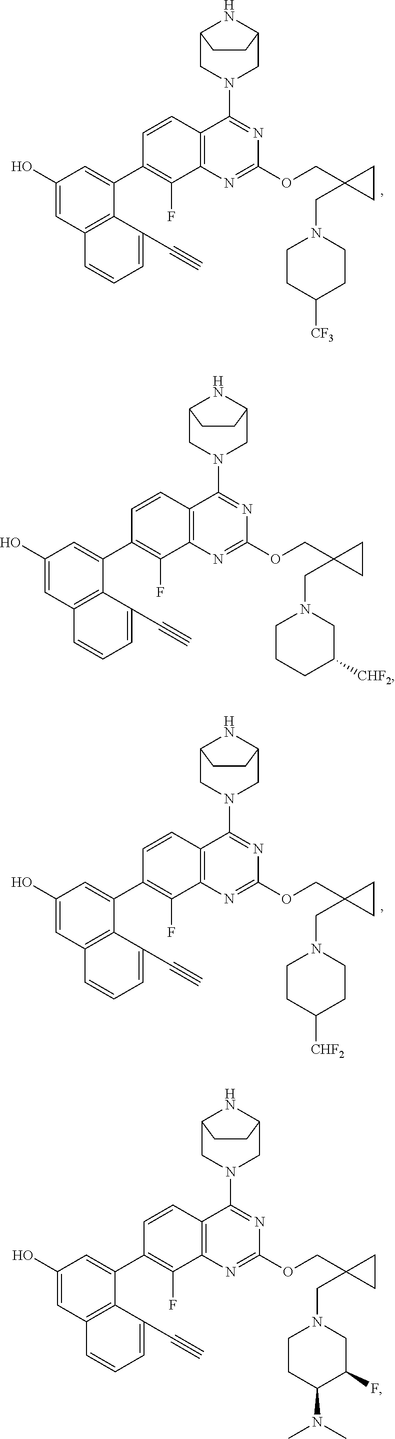 [SMILES: C#Cc1cccc2cc(O)cc(-c3ccc4c(N5CC6CCC(C5)N6)nc(OCC5(CN6CCC(C(F)(F)F)CC6)CC5)nc4c3F)c12 ; C#Cc1cccc2cc(O)cc(-c3ccc4c(N5CC6CCC(C5)N6)nc(OCC5(CN6CCC[C@@H](C(F)F)C6)CC5)nc4c3F)c12 ; C#Cc1cccc2cc(O)cc(-c3ccc4c(N5CC6CCC(C5)N6)nc(OCC5(CN6CCC(C(F)F)CC6)CC5)nc4c3F)c12 ; C#Cc1cccc2cc(O)cc(-c3ccc4c(N5CC6CCC(C5)N6)nc(OCC5(CN6CC[C@H](N(C)C)[C@H](F)C6)CC5)nc4c3F)c12]

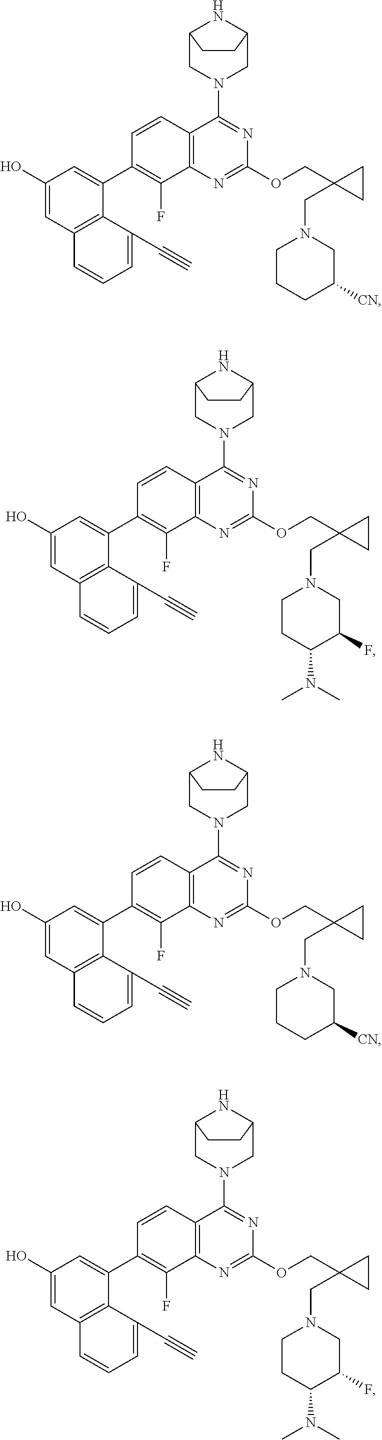 [SMILES: C#Cc1cccc2cc(O)cc(-c3ccc4c(N5CC6CCC(C5)N6)nc(OCC5(CN6CCC[C@@H](C#N)C6)CC5)nc4c3F)c12 ; C#Cc1cccc2cc(O)cc(-c3ccc4c(N5CC6CCC(C5)N6)nc(OCC5(CN6CC[C@@H](N(C)C)[C@H](F)C6)CC5)nc4c3F)c12 ; C#Cc1cccc2cc(O)cc(-c3ccc4c(N5CC6CCC(C5)N6)nc(OCC5(CN6CCC[C@H](C#N)C6)CC5)nc4c3F)c12 ; C#Cc1cccc2cc(O)cc(-c3ccc4c(N5CC6CCC(C5)N6)nc(OCC5(CN6CC[C@@H](N(C)C)[C@@H](F)C6)CC5)nc4c3F)c12]

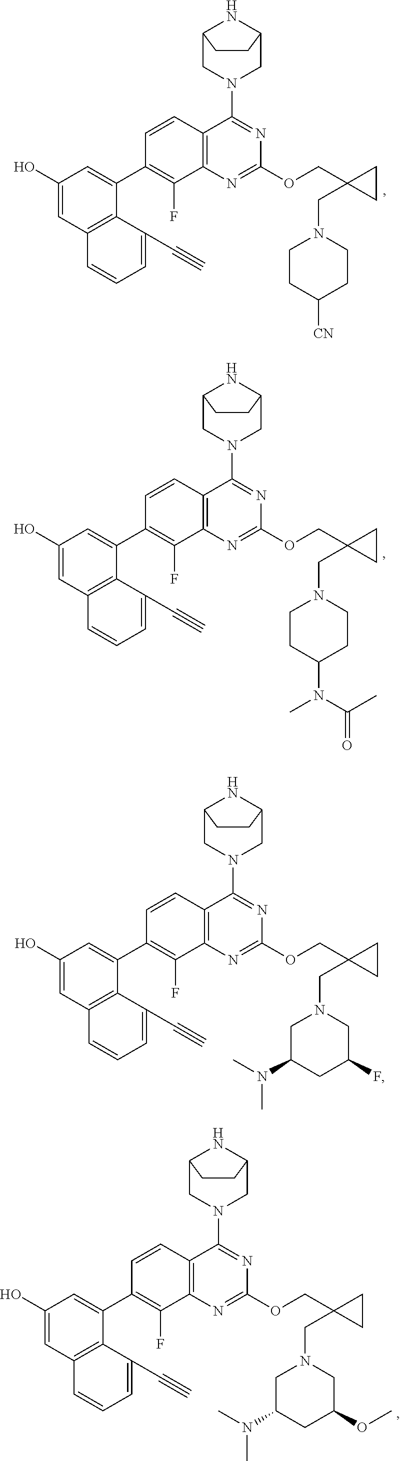 [SMILES: C#Cc1cccc2cc(O)cc(-c3ccc4c(N5CC6CCC(C5)N6)nc(OCC5(CN6CCC(C#N)CC6)CC5)nc4c3F)c12 ; C#Cc1cccc2cc(O)cc(-c3ccc4c(N5CC6CCC(C5)N6)nc(OCC5(CN6CCC(N(C)C(C)=O)CC6)CC5)nc4c3F)c12 ; C#Cc1cccc2cc(O)cc(-c3ccc4c(N5CC6CCC(C5)N6)nc(OCC5(CN6C[C@@H](F)C[C@@H](N(C)C)C6)CC5)nc4c3F)c12 ; C#Cc1cccc2cc(O)cc(-c3ccc4c(N5CC6CCC(C5)N6)nc(OCC5(CN6C[C@@H](OC)C[C@H](N(C)C)C6)CC5)nc4c3F)c12]

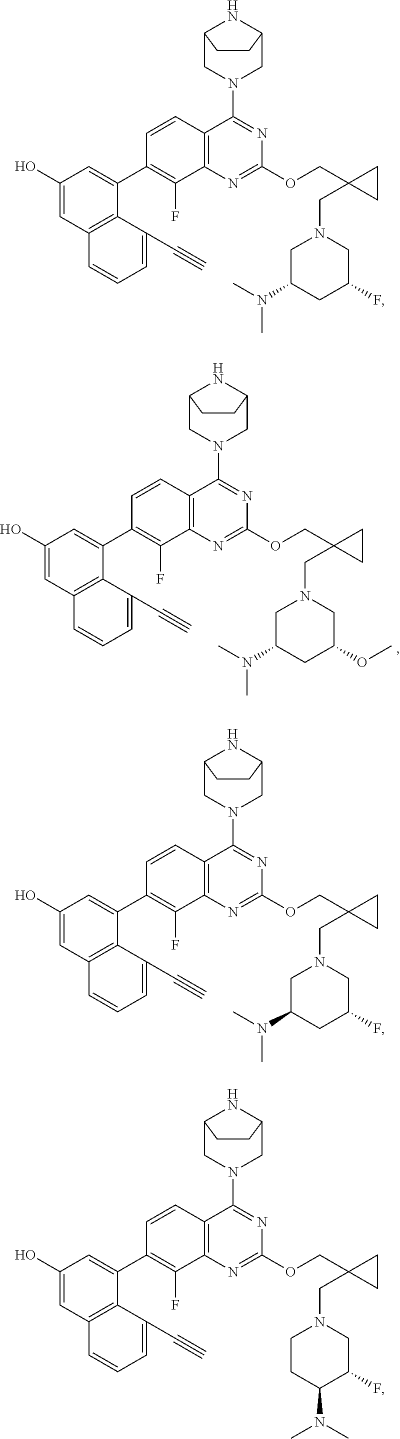 [SMILES: C#Cc1cccc2cc(O)cc(-c3ccc4c(N5CC6CCC(C5)N6)nc(OCC5(CN6C[C@H](F)C[C@H](N(C)C)C6)CC5)nc4c3F)c12 ; C#Cc1cccc2cc(O)cc(-c3ccc4c(N5CC6CCC(C5)N6)nc(OCC5(CN6C[C@H](OC)C[C@H](N(C)C)C6)CC5)nc4c3F)c12 ; C#Cc1cccc2cc(O)cc(-c3ccc4c(N5CC6CCC(C5)N6)nc(OCC5(CN6C[C@H](F)C[C@@H](N(C)C)C6)CC5)nc4c3F)c12 ; C#Cc1cccc2cc(O)cc(-c3ccc4c(N5CC6CCC(C5)N6)nc(OCC5(CN6CC[C@H](N(C)C)[C@@H](F)C6)CC5)nc4c3F)c12]

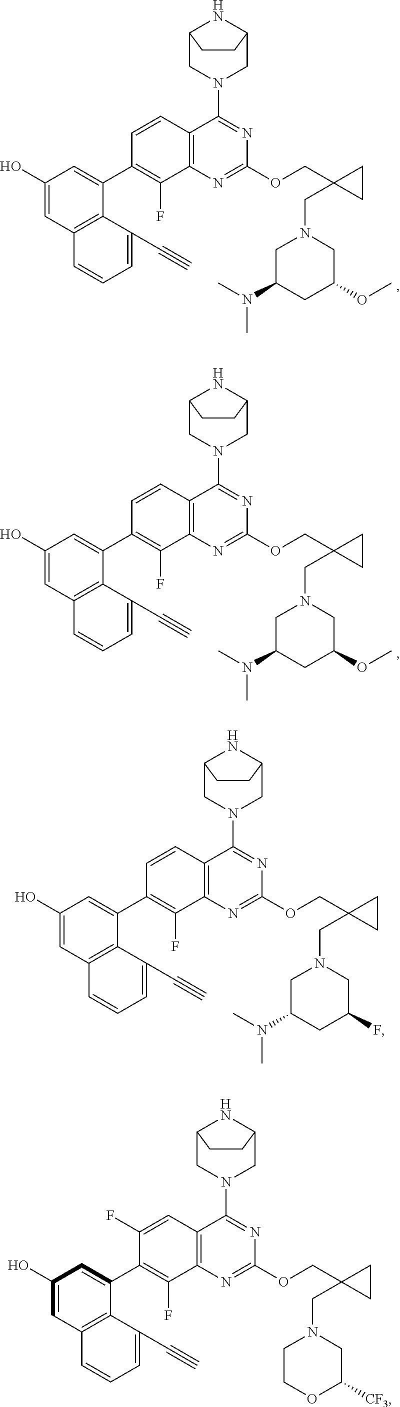

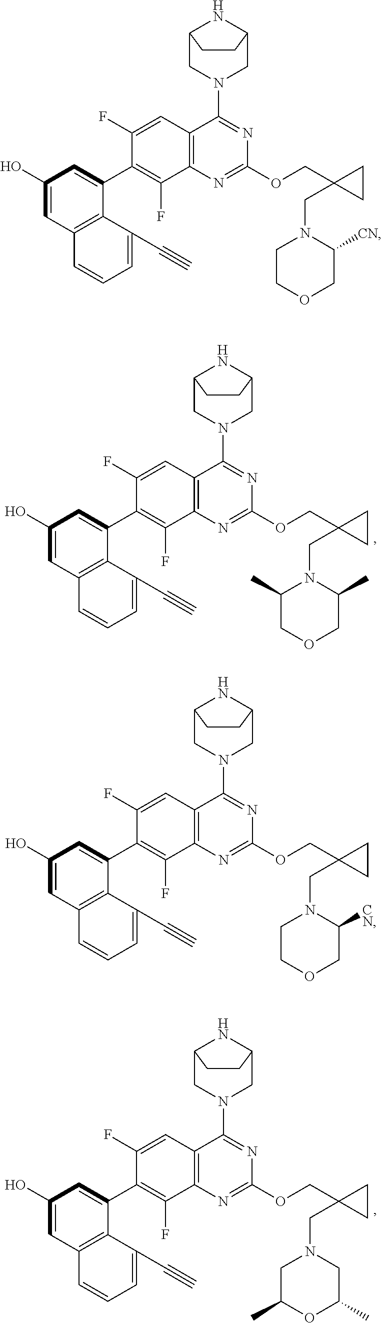

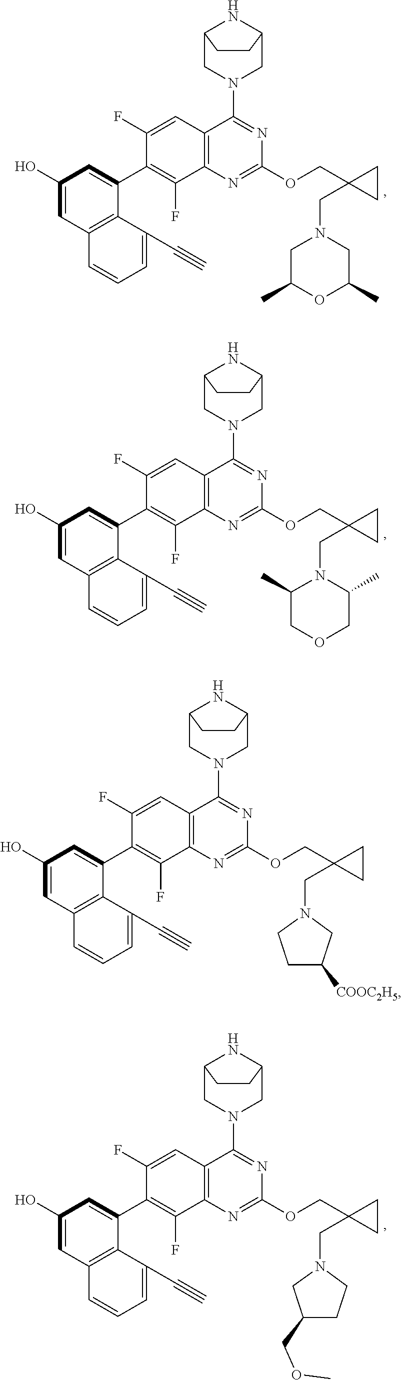

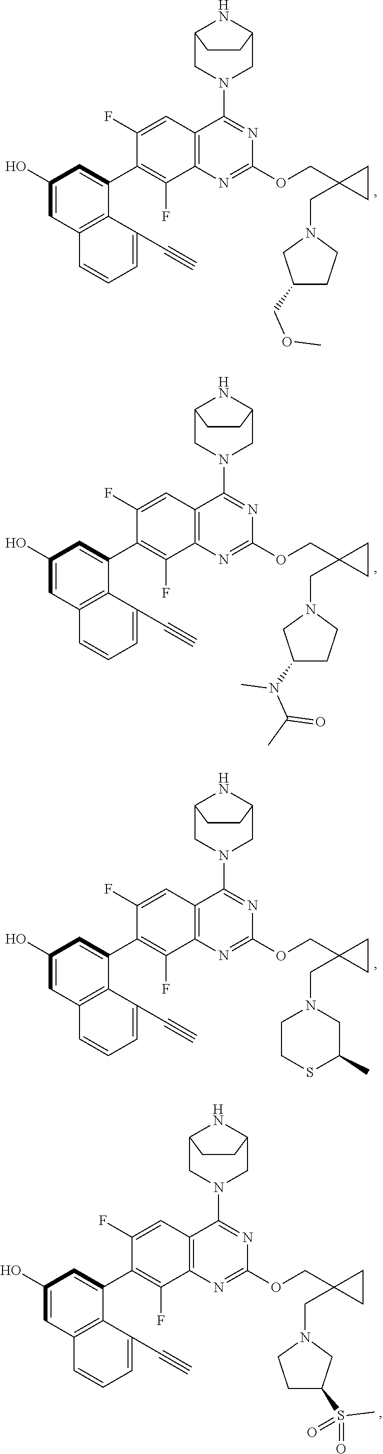

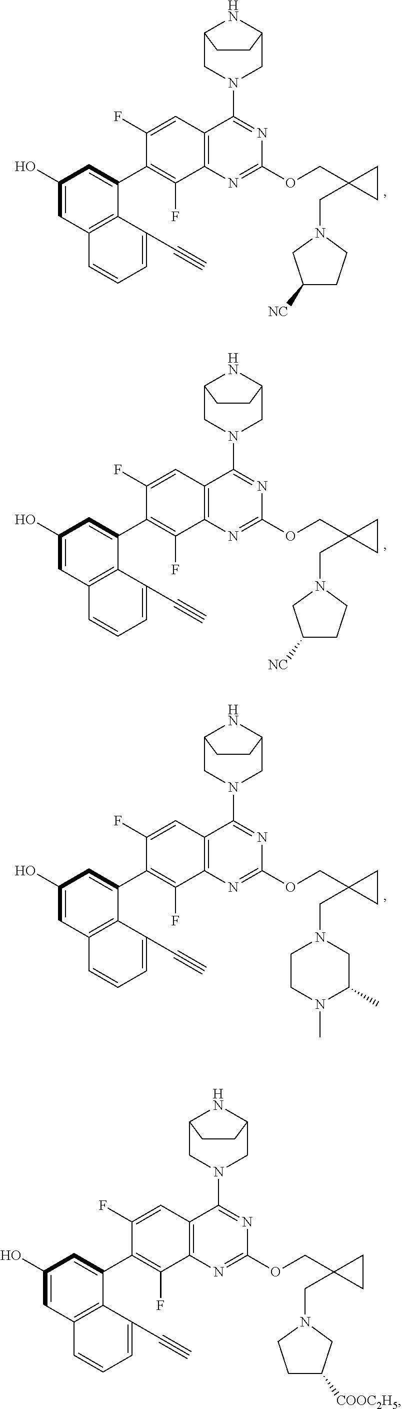

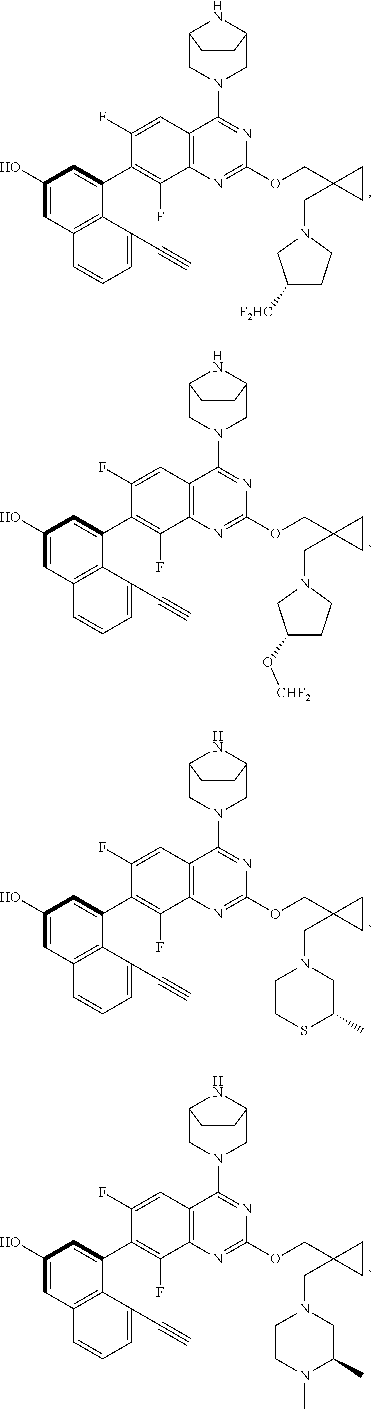

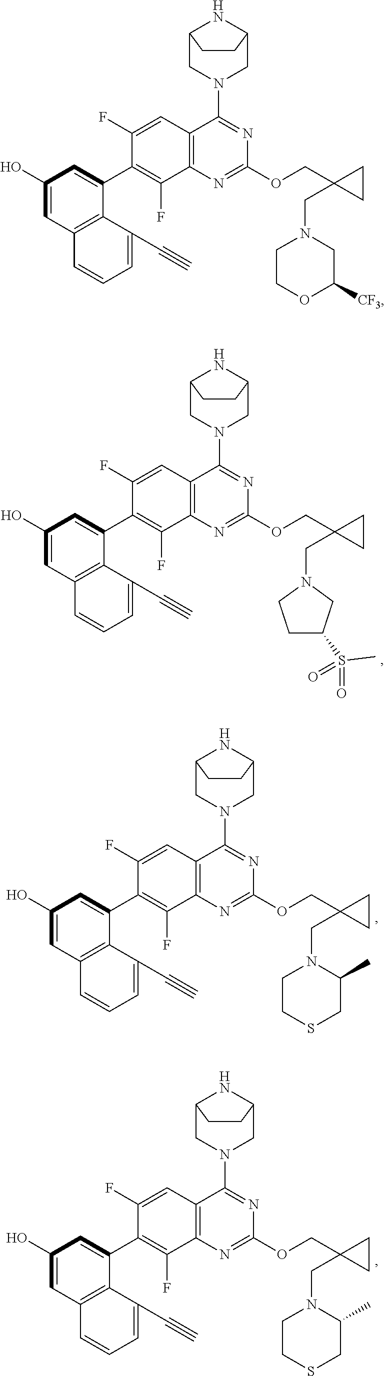

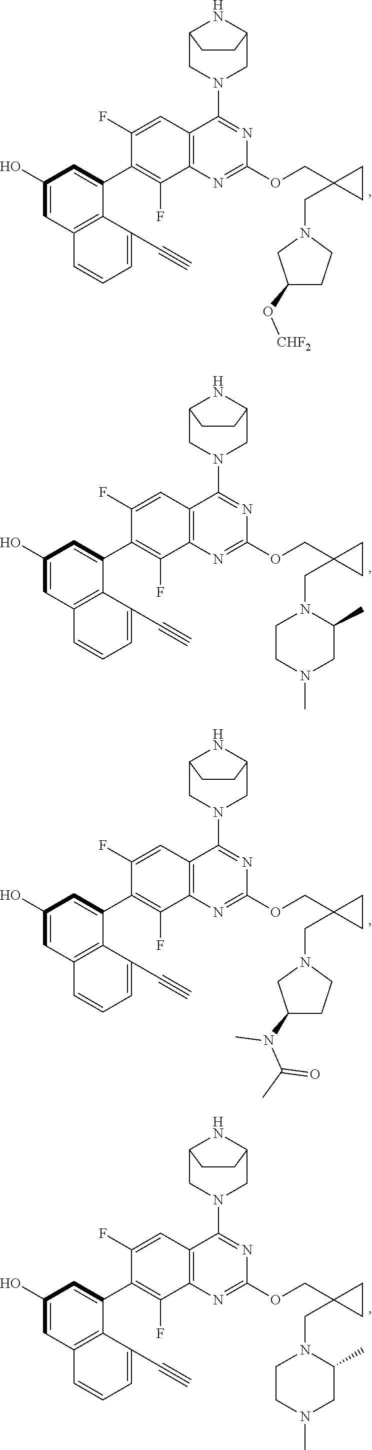

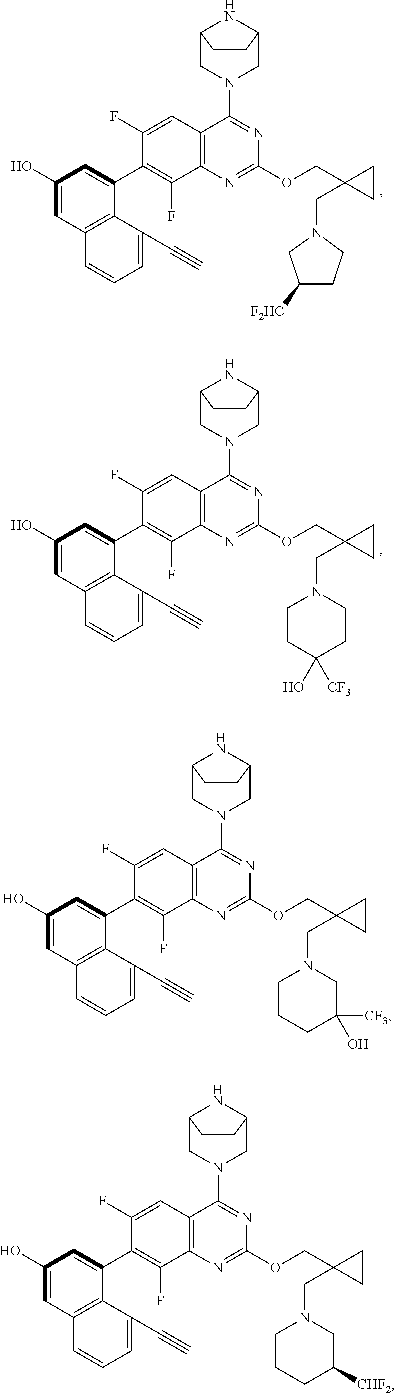

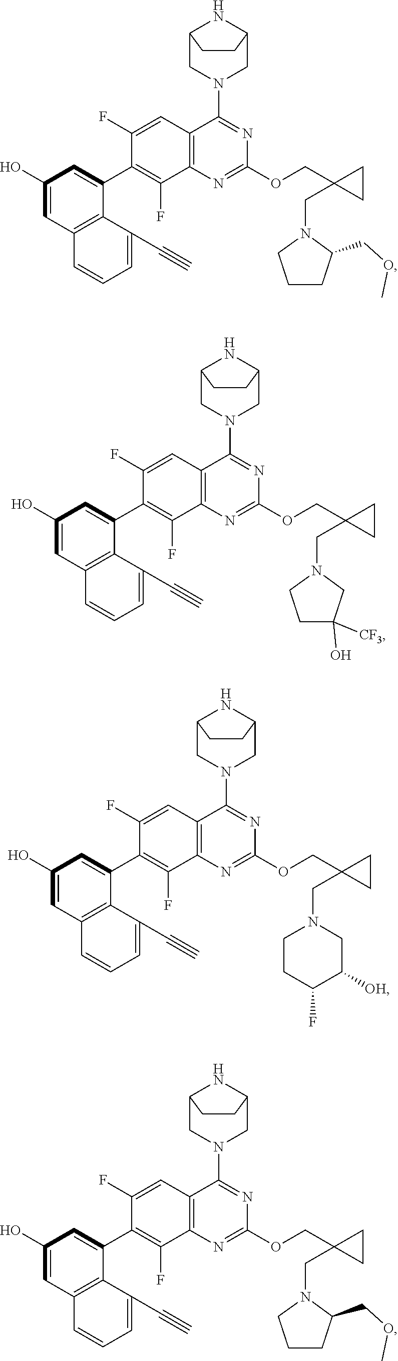

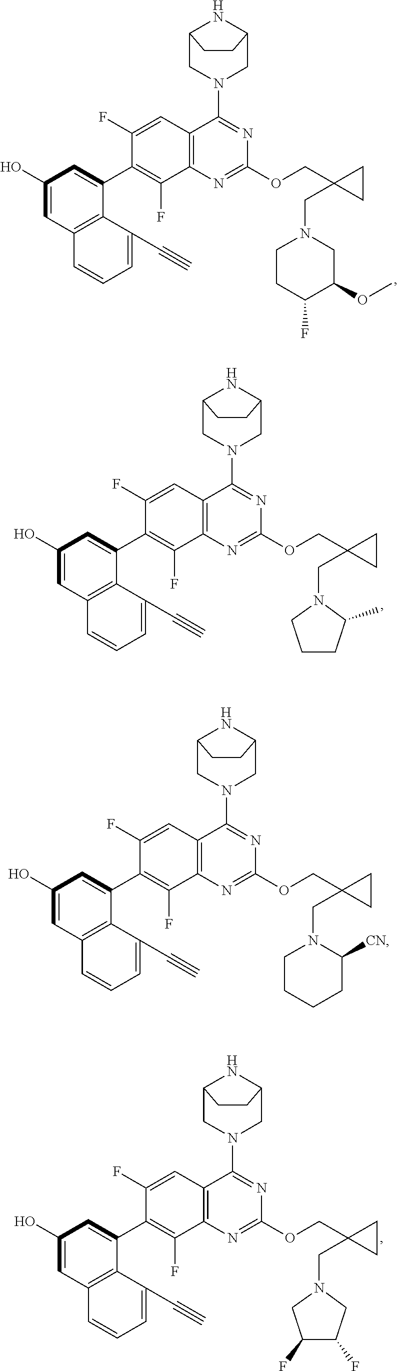

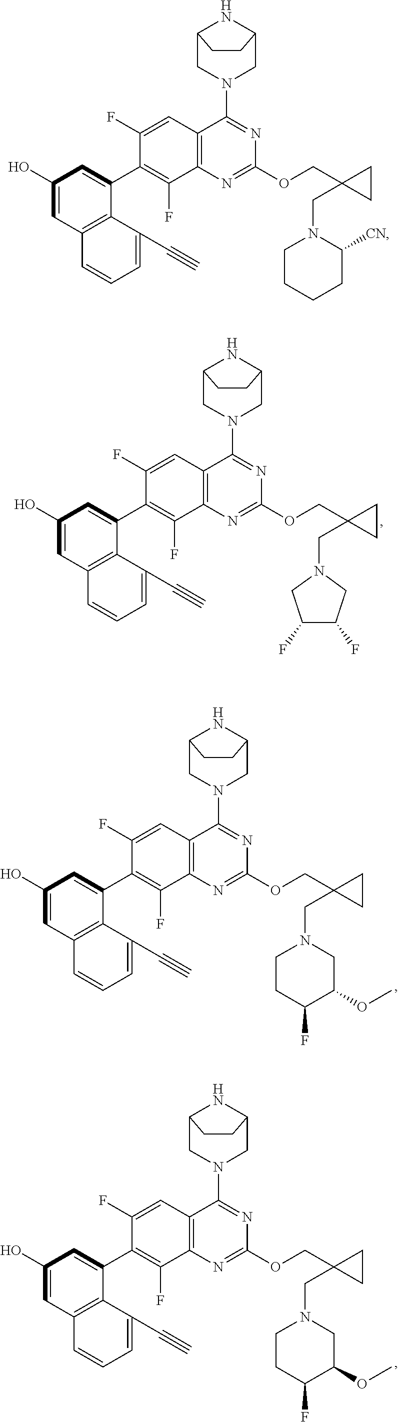

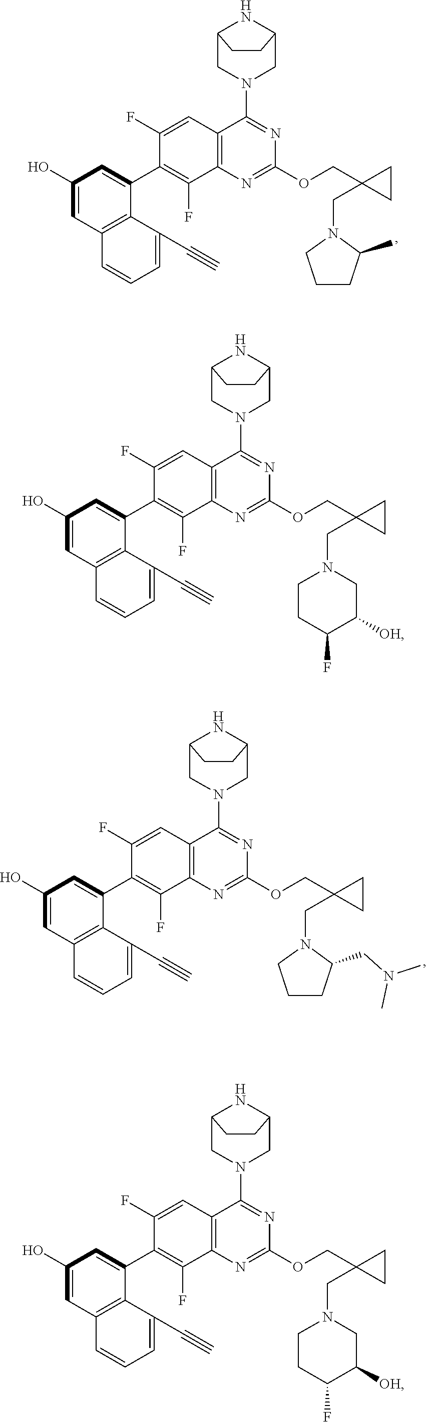

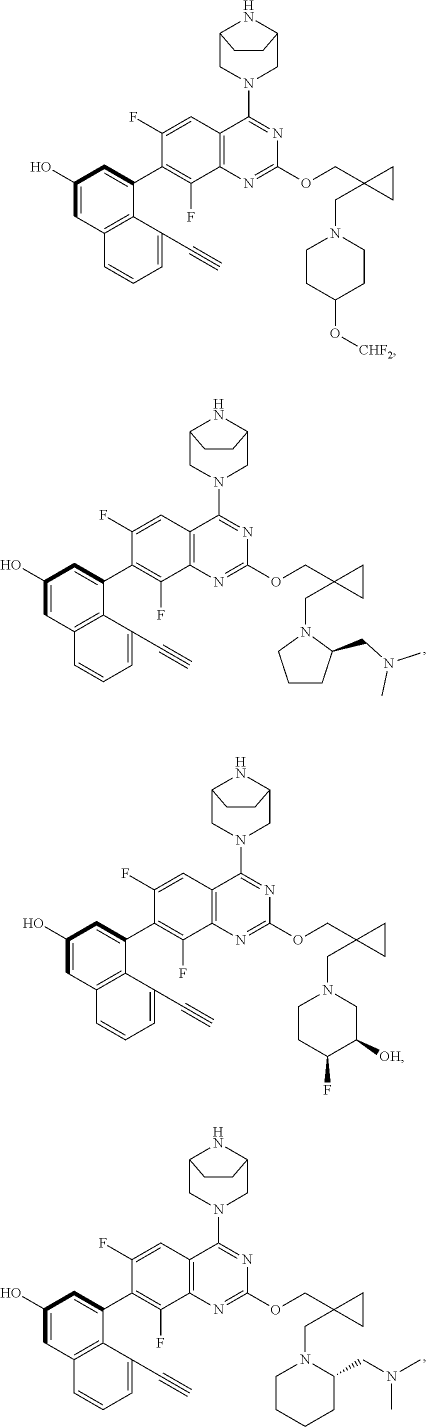

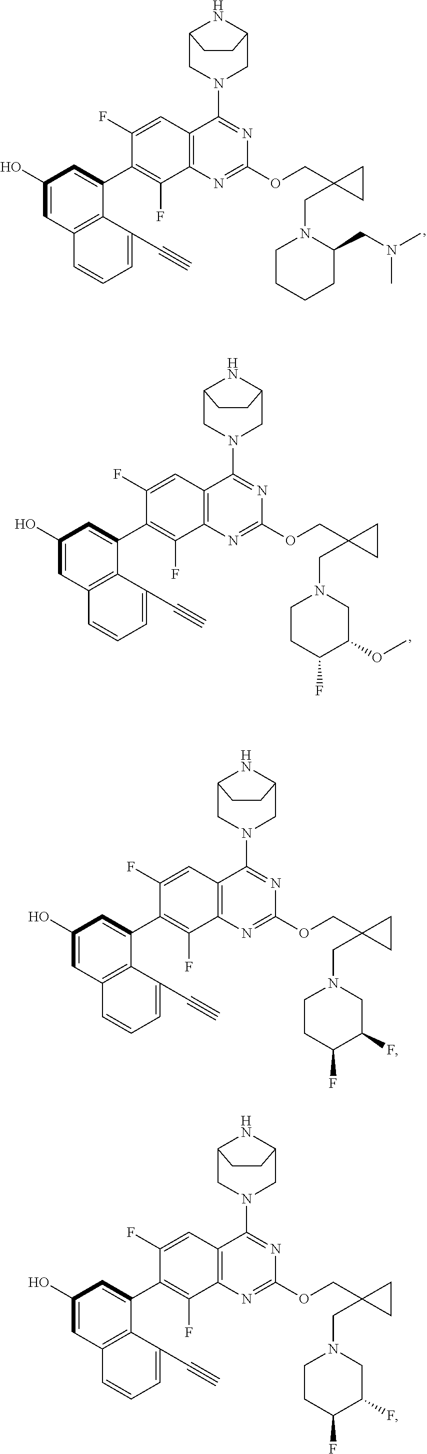

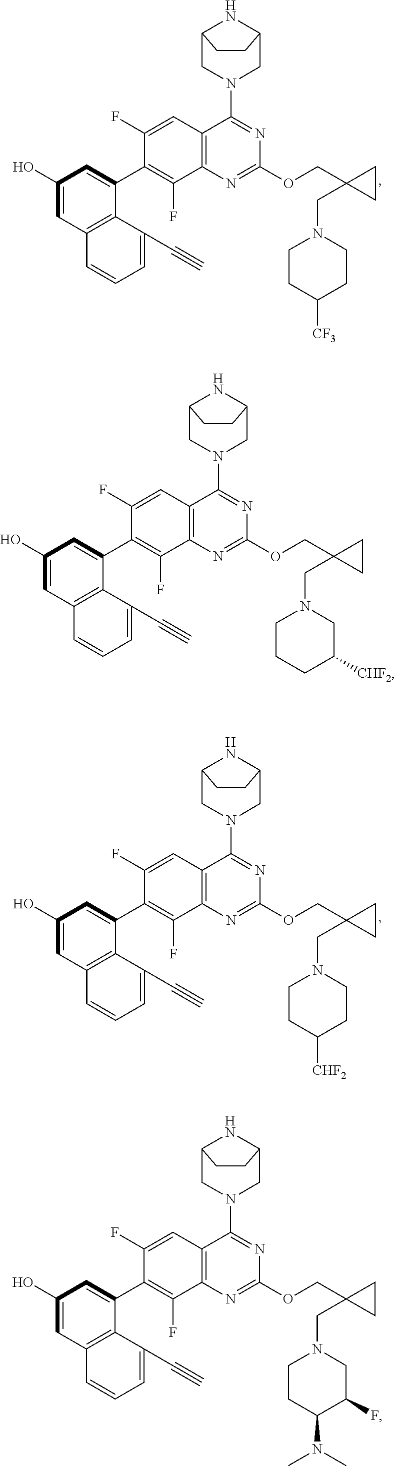

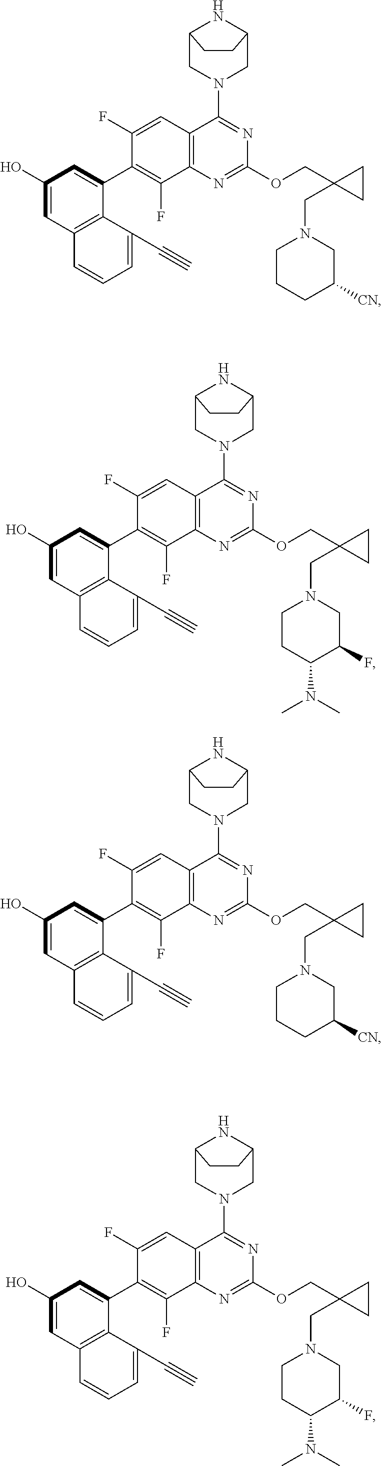

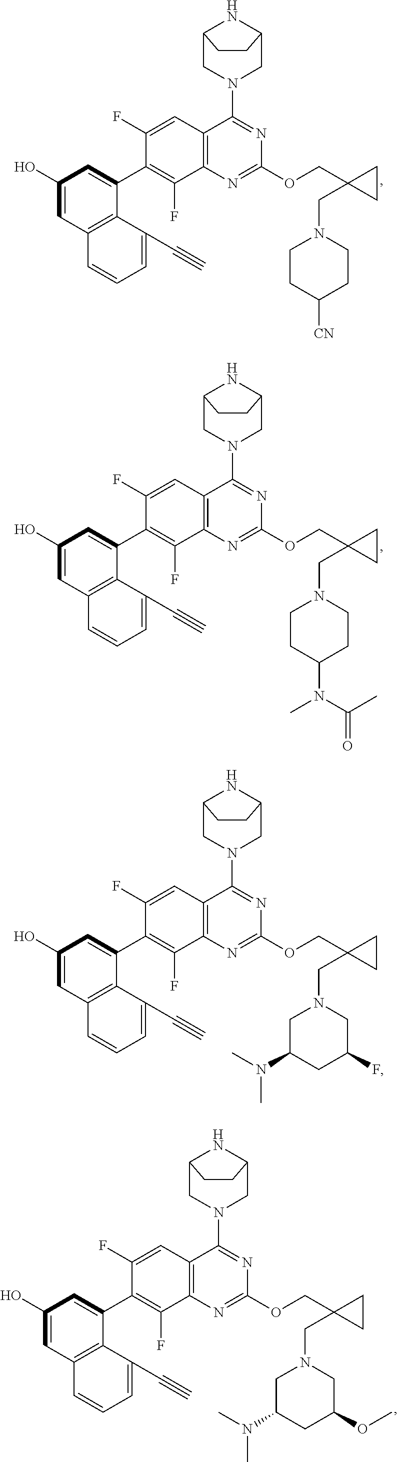

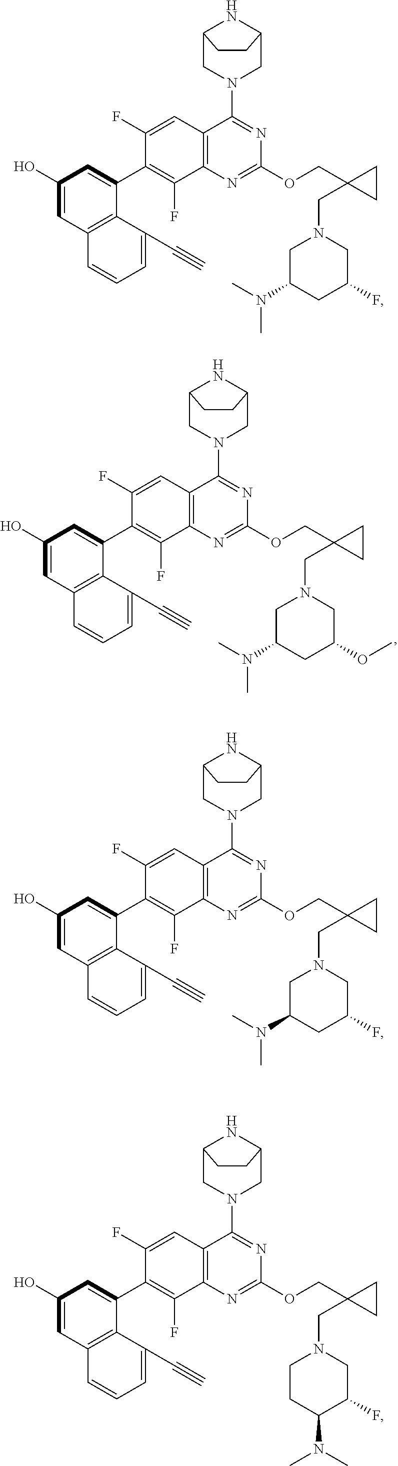

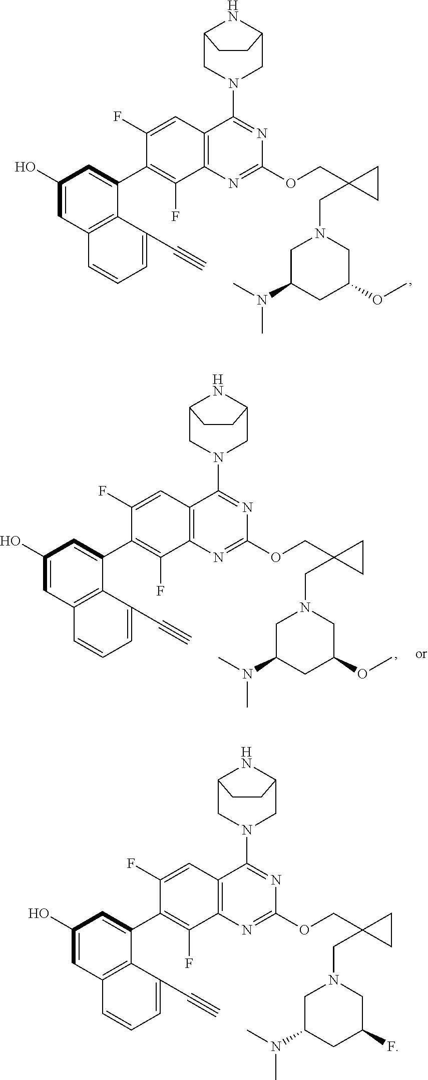

[20] A proteolysis targeting chimeric (PROTAC) compound acting as a ubiquitination and degradation modulator of KRAS G12D protein, wherein, said PROTAC compound is formed by joining the compound of formula (I), a stereoisomer thereof, an atropisomer thereof, a deuterated derivatives thereof, a tautomer thereof, or a pharmaceutically acceptable salt thereof of any one of [1] to [19] with an E3 ubiquitin ligase ligand with or without a linker.

[21] A prodrug of a compound a stereoisomer thereof, an atropisomer thereof, a deuterated derivative thereof, a tautomer thereof, or a pharmaceutically acceptable salt thereof of the present invention, or the or the proteolysis targeting chimeric (PROTAC) compound of the present application.

[22] A pharmaceutical composition comprising a compound, a stereoisomer thereof, an atropisomer thereof, a deuterated derivative thereof, a tautomer thereof, or a pharmaceutically acceptable salt thereof of the present invention, or the or the proteolysis targeting chimeric (PROTAC) compound of the present application, or a prodrug of the present invention, and at least one pharmaceutically acceptable excipient, preferably, the said compound in a weight ratio to the said excipient within the range from about 0.0001 to about 10.

[23] Use of a compound, a stereoisomer thereof, an atropisomer thereof, a deuterated derivative thereof, a tautomer thereof, or a pharmaceutically acceptable salt thereof of the present invention; or the proteolysis targeting chimeric (PROTAC) compound of the present invention, or the prodrug of the present invention, or the pharmaceutical composition of the present invention for the manufacture of a medicament for the treatment of diseases or conditions related to KRAS G12D mutant protein, preferably, the diseases or conditions related to KRAS G12D mutant protein is cancer related to KRAS G12D mutant protein, preferably, the cancer is selected from pancreatic cancer, colorectal cancer, endometrial cancer or lung cancer, preferably, the lung cancer is selected from non-small cell lung cancer or small cell lung cancer.

[24] A method of treating a subject having a diseases or conditions related to KRAS G12D mutant protein, said method comprising administering to the subject a therapeutically effective amount of a compound, a stereoisomer thereof, an atropisomer thereof, a deuterated derivative thereof, a tautomer thereof, or a pharmaceutically acceptable salt thereof of the present invention; or the proteolysis targeting chimeric (PROTAC) compound of the present invention; or a prodrug of the present invention; or the pharmaceutical composition of the present invention, preferably, the diseases or conditions related to KRAS G12D mutant protein is cancer related to KRAS G12D mutant protein, preferably, the cancer is selected from pancreatic cancer, colorectal cancer, endometrial cancer or lung cancer, preferably, the lung cancer is selected from non-small cell lung cancer or small cell lung cancer.

[25] A compound, a stereoisomer thereof, an atropisomer thereof, a deuterated derivative thereof, a tautomer thereof, or a pharmaceutically acceptable salt thereof of the present invention; or the proteolysis targeting chimeric (PROTAC) compound of the present invention; or a prodrug of the present invention; or the pharmaceutical composition of the present invention for use in the treatment of diseases or conditions related to KRAS G12D mutant protein, preferably, the diseases or conditions related to KRAS G12D mutant protein is cancer related to KRAS G12D mutant protein, preferably, the cancer is selected from pancreatic cancer, colorectal cancer, endometrial cancer or lung cancer, preferably, the lung cancer is selected from non-small cell lung cancer or small cell lung cancer.

[26] Use of a compound, a stereoisomer thereof, an atropisomer thereof, a deuterated derivative thereof, a tautomer thereof, or a pharmaceutically acceptable salt thereof of the present invention as a targeting KRAS G12D protein ligand in a PROTAC compound acting as a ubiquitination and degradation modulator of KRAS G12D protein.

### Definition

Unless defined otherwise, all technical and scientific terms used herein have the same meaning as is commonly understood by one of skill in the art to which this invention belongs. All patents, patent applications, and publications referred to herein are incorporated by reference.

The term “halogen” or “halo”, as used herein, unless otherwise indicated, means fluoro, chloro, bromo or iodo. The preferred halogen groups include —F, —Cl and —Br.

The term “alkyl”, as used herein, unless otherwise indicated, includes saturated monovalent hydrocarbon radicals having straight or branched. For example, alkyl radicals include methyl, ethyl, propyl, isopropyl, n-butyl, isobutyl, sec-butyl, t-butyl, n-pentyl, 3-(2-methyl)butyl, 2-pentyl, 2-methylbutyl, neopentyl, n-hexyl, 2-hexyl and 2-methylpentyl. Similarly, C₁₋₆ as in C₁₋₆alkyl is defined to identify the group as having 1, 2, 3, 4, 5 or 6 carbon atoms in a linear or branched arrangement.

The term “alkylene” means a difunctional group obtained by removal of a hydrogen atom from an alkyl group defined above. For example, methylene (i.e., —CH₂—), ethylene (i.e., —CH₂—CH₂— or —CH(CH₃)—) and propylene (i.e., —CH₂—CH₂—CH₂—, —CH(—CH₂—CH₃)— or —CH₂—CH(CH₃)—).

The term “alkenyl” means a straight or branch-chained hydrocarbon radical containing one or more double bonds and typically from 2 to 20 carbon atoms in length. For example, “C₂₋₆alkenyl” contains from 2 to 6 carbon atoms. Alkenyl group include, but are not limited to, for example, ethenyl, propenyl, butenyl, 2-methyl-2-buten-1-yl, heptenyl, octenyl and the like.

The term “alkynyl” contains a straight or branch-chained hydrocarbon radical containing one or more triple bonds and typically from 2 to 20 carbon atoms in length. For example, “C₂₋₆alkynyl” contains from 2 to 6 carbon atoms. Representative alkynyl groups include, but are not limited to, for example, ethynyl, 1-propynyl, 1-butynyl, heptynyl, octynyl and the like.

The term “alkoxy” radicals are oxygen ethers formed from the previously described alkyl groups.

The term “aryl”, as used herein, unless otherwise indicated, refers to an unsubstituted or substituted mono or polycyclic aromatic ring system containing carbon ring atoms. The preferred aryls are mono cyclic or bicyclic 6-10 membered aromatic ring systems. Phenyl and naphthyl are preferred aryls.

The term “heterocyclyl”, as used herein, unless otherwise indicated, refers to unsubstituted and substituted mono or polycyclic non-aromatic ring system containing one or more heteroatoms, which comprising monocyclic heterocyclyl ring, bicyclic heterocyclyl ring, bridged heterocyclyl ring, fused heterocyclyl ring or sipro heterocyclyl ring. Preferred heteroatoms include N, O, and S, including N-oxides, sulfur oxides, and dioxides. Preferably, the ring is three to ten membered and is either fully saturated or has one or more degrees of unsaturation. Multiple degrees of substitution, preferably one, two or three, are included within the present definition. Examples of such heterocyclyl groups include, but are not limited to azetidinyl, pyrrolidinyl, piperidinyl, piperazinyl, oxopiperazinyl, oxopiperidinyl, oxoazepinyl, azepinyl, tetrahydrofuranyl, dioxolanyl, tetrahydroimidazolyl, tetrahydrothiazolyl, tetrahydrooxazolyl, tetrahydropyranyl, morpholinyl, thiomorpholinyl, thiamorpholinylsulfoxide, thiamorpholinylsulfone and oxadiazolyl.

The term “heteroaryl”, as used herein, unless otherwise indicated, represents an aromatic ring system containing carbon(s) and at least one heteroatom. Heteroaryl may be monocyclic or polycyclic, substituted or unsubstituted. A monocyclic heteroaryl group may have 1 to 4 heteroatoms in the ring, while a polycyclic heteroaryl may contain 1 to 10 hetero atoms. A polycyclic heteroaryl ring may contain fused, spiro or bridged ring junction, for example, bycyclicheteroaryl is a polycyclic heteroaryl. Bicyclic heteroaryl rings may contain from 8 to 12 member atoms. Monocyclic heteroaryl rings may contain from 5 to 8 member atoms (carbons and heteroatoms). Examples of heteroaryl groups include, but are not limited to thienyl, furanyl, imidazolyl, isoxazolyl, oxazolyl, pyrazolyl, pyrrolyl, thiazolyl, thiadiazolyl, triazolyl, pyridyl, pyridazinyl, indolyl, azaindolyl, indazolyl, benzimidazolyl, benzofuranyl, benzothienyl, benzisoxazolyl, benzoxazolyl, benzopyrazolyl, benzothiazolyl, benzothiadiazolyl, benzotriazolyladeninyl, quinolinyl or isoquinolinyl.

The term “carbocyclic” refers to a substituted or unsubstituted monocyclic ring, bicyclic ring, bridged ring, fused ring, sipiro ring non-aromatic ring system only containing carbon atoms. Exemplary “cycloalkyl” groups includes but not limited to, cyclopropyl, cyclobutyl, cyclopentyl, cyclohexyl and so on.

The term “one or more” refers to one or more than one. In some embodiments, “one or more” refers to 1, 2, 3, 4, 5 or 6. In some embodiments, “one or more” refers to 1, 2, 3 or 4. In some embodiments, “one or more” refers to 1, 2, or 3. In some embodiments, “one or more” refers to 1 or 2. In some embodiments, “one or more” refers to 1. In some embodiments, “one or more” refers to 2. In some embodiments, “one or more” refers to 3. In some embodiments, “one or more” refers to 4. In some embodiments, “one or more” refers to 5. In some embodiments, “one or more” refers to 6.

When one or more substituents (such as R_{S1}, R_{S2} or R_{S3}) are substituted on a ring in the present invention, it means that each of substituents may be respectively independently substituted on every ring atom of the ring including but not limited to a ring carbon atom or a ring nitrogen atom. In addition, when the ring is a polycyclic ring, such as a fused ring, a bridged ring or a spiro ring, each of substituents may be respectively independently substituted on every ring atom of the polycyclic ring though the substituents are drawn to one of the rings of the polycyclic ring. For example, the R_{S1} in the moiety of

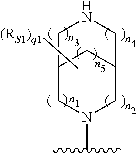

means each of R_{S1} is respectively independently substituted on every ring atom of the 8-membered bridged ring including every ring carbon atom or the ring nitrogen atom.

The term “oxo” refers to oxygen atom together with the attached carbon atom forms the group

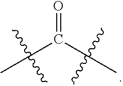 [SMILES: *C(*)=O]

The term “composition”, as used herein, is intended to encompass a product comprising the specified ingredients in the specified amounts, as well as any product which results, directly or indirectly, from combinations of the specified ingredients in the specified amounts. Accordingly, pharmaceutical compositions containing the compounds of the present invention as the active ingredient as well as methods of preparing the instant compounds are also part of the present invention. Furthermore, some of the crystalline forms for the compounds may exist as polymorphs and as such are intended to be included in the present invention. In addition, some of the compounds may form solvates with water (i.e., hydrates) or common organic solvents and such solvates are also intended to be encompassed within the scope of this invention.

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. When the compound of the present invention is basic, its corresponding salt can be conveniently prepared from pharmaceutically acceptable non-toxic acids, including inorganic and organic acids. Since the compounds in the present invention are intended for pharmaceutical use they are preferably provided in substantially pure form, for example at least 60% pure, more suitably at least 75% pure, especially at least 98% pure (% are on a weight for weight basis).

The present invention includes within its scope the prodrugs of the compounds of this invention. In general, such prodrugs will be functional derivatives of the compounds that are readily converted in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the subject. Conventional procedures for the selection and preparation of suitable prodrug derivatives are described, for example, in “Design of Prodrugs”, ed. H. Bundgaard, Elsevier, 1985.

It is intended that the definition of any substituent or variable at a particular location in a molecule be independent of its definitions elsewhere in that molecule. It is understood that substituents and substitution patterns on the compounds of this invention can be selected by one of ordinary skill in the art to provide compounds that are chemically stable and that can be readily synthesized by techniques known in the art as well as those methods set forth herein.

The present invention includes compounds described can contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof.

The present invention includes all stereoisomers of the compound and pharmaceutically acceptable salts thereof. Further, mixtures of stereoisomers as well as isolated specific stereoisomers are also included. During the course of the synthetic procedures used to prepare such compounds or in using racemization or epimerization procedures known to those skilled in the art, the products of such procedures can be a mixture of stereoisomers.

The term “stereoisomer” as used in the present invention refers to an isomer in which atoms or groups of atoms in the molecule are connected to each other in the same order but differ in spatial arrangement, including conformational isomers and conformational isomers. The configuration isomers include geometric isomers and optical isomers, and optical isomers mainly include enantiomers and diastereomers. The invention includes all possible stereoisomers of the compound.

Certain of the compounds provided herein may exist as atropisomers, which are conformational stereoisomers that occur when rotation about a single bond in the molecule is prevented, or greatly slowed, as a result of steric interactions with other parts of the molecule. The compounds provided herein include all atropisomers, both as pure individual atropisomer preparations, enriched preparations of each, or a non-specific mixture of each. Where the rotational barrier about the single bond is high enough, and interconversion between conformations is slow enough, separation and isolation of the isomeric species may be permitted.

The present invention is intended to include all isotopes of atoms occurring in the present compounds. Isotopes include those atoms having the same atomic number but different mass numbers. By way of general example and without limitation, isotopes of hydrogen include deuterium and tritium. The isotopes of hydrogen can be denoted as ¹H (hydrogen), ²H (deuterium) and ³H (tritium). They are also commonly denoted as D for deuterium and T for tritium. In the application, CD₃ denotes a methyl group wherein all of the hydrogen atoms are deuterium. Isotopes of carbon include ¹³C and ¹⁴C. Isotopically-labeled compounds of the invention can generally be prepared by conventional techniques known to those skilled in the art or by presses analogous to those described herein, using an appropriate isotopically-labeled reagent in place of the non-labeled reagent.

When a tautomer of the compound in the present invention exists, the present invention includes any possible tautomers and pharmaceutically acceptable salts thereof, and mixtures thereof, except where specifically stated otherwise.

When the compound in present invention and pharmaceutically acceptable salts thereof exist in the form of solvates or polymorphic forms, the present invention includes any possible solvates and polymorphic forms. A type of a solvent that forms the solvate is not particularly limited so long as the solvent is pharmacologically acceptable.

The pharmaceutical compositions of the present invention comprise a compound in present invention (or a pharmaceutically acceptable salt thereof) as an active ingredient, a pharmaceutically acceptable carrier and optionally other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the compounds in present invention or a prodrug or a metabolite or pharmaceutically acceptable salts thereof, of this invention can be combined as the active ingredient in intimate admixture with a pharmaceutical carrier according to conventional pharmaceutical compounding techniques. The carrier may take a wide variety of forms depending on the form of preparation desired for administration, e.g. oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compound represented by Formula (I) or a pharmaceutically acceptable salt thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and a compound or a pharmaceutically acceptable salt. The compounds of Formula (I) or pharmaceutically acceptable salts thereof can also be included in pharmaceutical compositions in combination with one or more other therapeutically active compounds.

The pharmaceutical carrier employed can be, for example, a solid, liquid or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen. In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably containing from about 0.05 mg to about 5 g of the active ingredient. For example, a formulation intended for the oral administration to humans may contain from about 0.5 mg to about 5 g of active agent, compounded with an appropriate and convenient amount of carrier material which may vary from about 0.05 to about 95 percent of the total composition. Unit dosage forms will generally contain between from about 0.01 mg to about 2 g of the active ingredient, typically 0.01 mg, 0.02 mg, 1 mg, 2 mg, 3 mg, 4 mg, 5 mg, 6 mg, 7 mg, 8 mg, 9 mg, 10 mg, 25 mg, 50 mg, 100 mg, 200 mg, 300 mg, 400 mg, 500 mg, 600 mg, 800 mg, 1000 mg, 1500 mg or 2000 mg.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder or the like. Further, the compositions can be in a form suitable for use in transdermal devices. These formulations may be prepared, utilizing a compound represented by Formula (I) of this invention or a pharmaceutically acceptable salt thereof, via conventional processing methods. As an example, a cream or ointment is prepared by admixing hydrophilic material and water, together with about 0.05 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including antioxidants) and the like. Furthermore, other adjuvants can be included to render the formulation isotonic with the blood of the intended recipient. Compositions containing a compound described by Formula (I) or pharmaceutically acceptable salts thereof may also be prepared in powder or liquid concentrate form.

Generally, dosage levels on the order of from about 0.001 mg/kg to about 150 mg/kg of body weight per day are useful in the treatment of the above-indicated conditions or alternatively about 0.05 mg to about 7 g per patient per day. For example, inflammation, cancer, psoriasis, allergy/asthma, disease and conditions of the immune system, disease and conditions of the central nervous system (CNS), may be effectively treated by the administration of from about 0.001 to 50 mg of the compound per kilogram of body weight per day or alternatively about 0.05 mg to about 3.5 g per patient per day.

It is understood, however, that the specific dose level for any particular patient will depend upon a variety of factors including the age, body weight, general health, sex, diet, time of administration, route of administration, rate of excretion, drug combination and the severity of the particular disease undergoing therapy.

These and other aspects will become apparent from the following written description of the invention.

Methods of Preparation

Compounds of the present invention can be synthesized from commercially available reagents using the synthetic methods and reaction schemes described herein. The examples which outline specific synthetic route and the generic schemes below are meant to provide guidance to the ordinarily skilled synthetic chemist, who will readily appreciate that the solvent, concentration, reagent, protecting group, order of synthetic steps, time, temperature, and the like can be modified as necessary, well within the skill and judgment of the ordinarily skilled artisan.

### EXAMPLES

The following Examples are provided to belier illustrate the present invention. All parts and percentages are by weight and all temperatures are degrees Celsius, unless explicitly stated otherwise. The following abbreviations have been used in the examples:

| | |
|---|---|
| DMF | N,N-Dimethylformamide |
| EA/EtOAc | Ethyl acetate |
| Hex | Hexane |
| MeOH | Methanol |
| DCM | Dichloromethane |
| DCE | 1,2-Dichloroethane |
| EtOH | Ethanol |
| THF | Tetrahydrofuran |
| DIEA/DIPEA | N,N-Diisopropylethylamine |
| Pd(PPh₃)₄ | Tetrakis(triphenylphosphine)palladium |
| Pd(dppf)Cl₂ | [1,1′-Bis(diphenylphosphino)ferrocene] |
| | dichloropalladium(II) |
| TFA | 2,2,2-Trifluoroacetic acid |
| CAN/CH₃CN | Acetonitrile |
| TEA | Triethanolamine |
| NIS | N-Iodosuccinimide |
| DMSO | Dimethyl sulfoxide |
| NCS | N-chlorosuccinimide |
| TBSCl | Tert-butyldimethylsilyl chloride\| |
| TMSCl | Trimethylsilyl chloride |
| MOMCl | Methoxymethyl chloride |
| MsCl | Methanesulfonyl chloride |
| LAH | Lithium aluminum hydride |
| LDA | Lithium diisopropylamide |
| LIHMDS | Lithium hexamethyldisilazide |
| B₂(Pin)₂ | Bis(pinacolato)diboron |
| NFSI | N-Fluorobenzenesulfonimide |
| MTBE | Methyl tert-butyl ether |
| DMAP | N,N-dimethylpyridin-4-amine |
| DABCO | N,N-dimethylethanolamine |
| TABF | Tetrabutylammonium fluoride |
| M-CPBA | 3-Chloroperbenzoic acid |
| NMP | N-methylpyrrolidone |
| RT/R.T | Room temperature |
| min(s) | minute(s) |
| h/hr(s) | hour(s) |
| aq | aqueous |
| Sat. | saturated |
| TLC | Thin layer chromatography |
| Prep-TLC | Preparative thin layer chromatography |
| MOMO | Methoxymethoxy |
| TIPS | Triisopropylsilyl |
| IPA.M, MIPA, or | Isopropylamine |
| IPAMine | |

### Preparation of the Intermediates

The intermediate (INT) was synthesized by the following procedures.

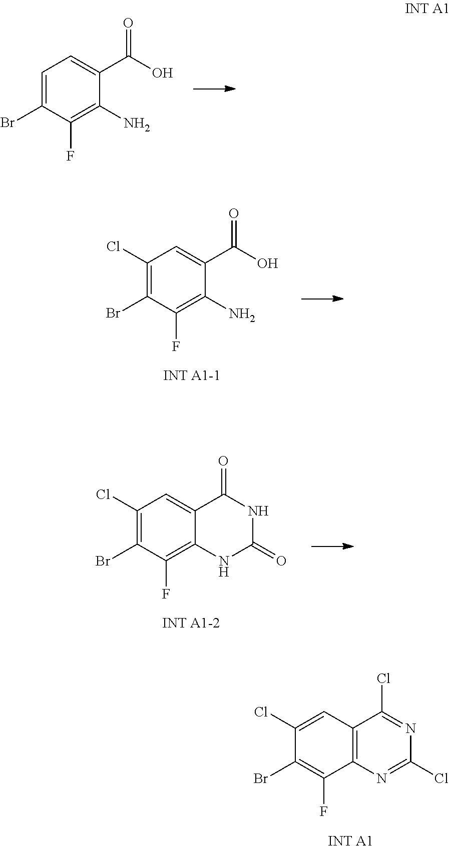

INT A1 was synthesized with 2-amino-4-bromo-3-fluorobenzoic acid as starting material through using conventional preparation method.

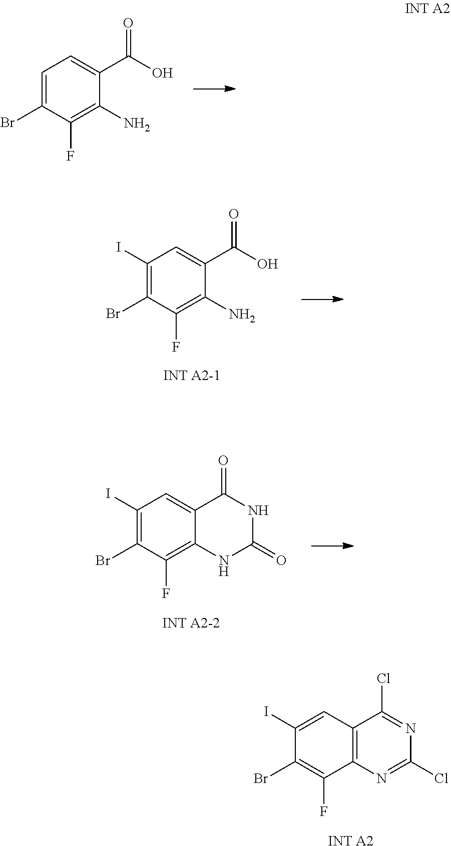

INT A2 was synthesized with 2-amino-4-bromo-3-fluorobenzoic acid as starting material through using conventional preparation method.

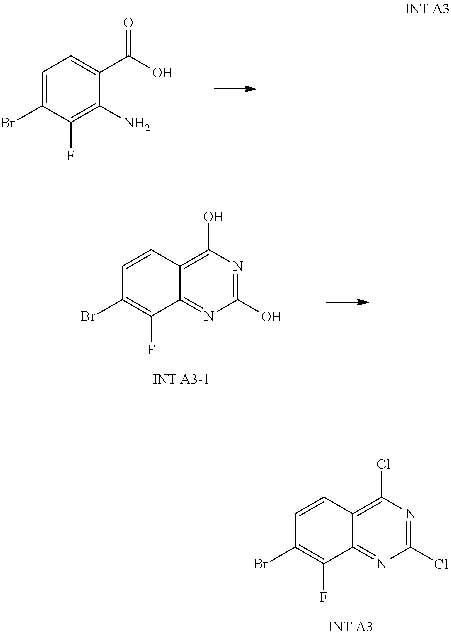

To a solution of 2-amino-4-bromo-3-fluorobenzoic acid (40.40 g, 172.63 mmol) in DCM (650 mL) was added dropwise sulfurisocyanatidic chloride (57.46 g, 405.98 mmol) at 0° C. The reaction mixture was stirred at RT for 5 hours under nitrogen atmosphere. The mixture was concentrated under reduced pressure. The residue was mixed with hydrochloric acid (6 M, 600 mL) and stirred at 45° C. overnight under nitrogen atmosphere. The reaction mixture was filtered and the filter cake was washed with water (200 mL), Hex (200 mL), and dried at 45° C. for overnight under vacuum to afford the desired product (INT A3-1, 37.63 g, 145.27 mmol). MS m/z: 257/259 [M−1].

To a solution of INT A3-1 (37.63 g, 145.27 mmol) in phosphorus oxychloride (115 mL) was added DIEA (46.24 g, 357.78 mmol) dropwise at 0° C. The reaction mixture was stirred at 110° C. for 2 hours under nitrogen atmosphere. The mixture was concentrated under vacuum. The residue was dissolved in DCM (600 mL) and washed with water (400 mL). The organic phase was concentrated under vacuum and the residue was suspended in Hex:EA (400 mL, 20:1, v/v). The mixture was filtrated and the filter cake was washed with Hex (100 mL), dried at 40° C. for overnight under vacuum to afford the desired product (INT A3, 31.29 g, 105.74 mmol) as a yellow solid.

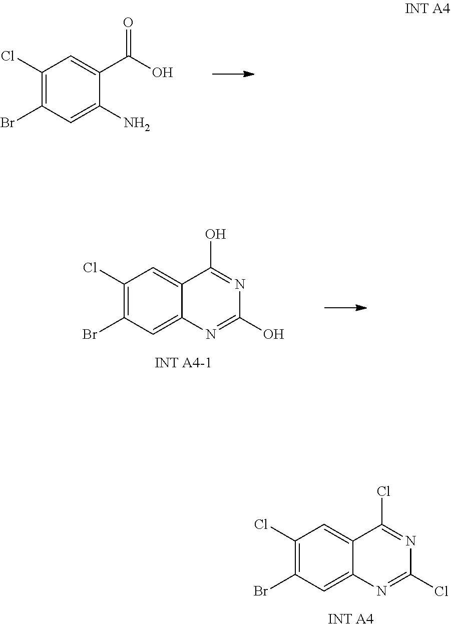

Sulfurisocyanatidic chloride (6.55 g, 46.28 mmol) was added dropwise to a solution of 2-amino-4-bromo-5-chlorobenzoic acid (5.12 g, 20.44 mmol) in DCM (30 mL) at 0° C. The reaction mixture was stirred for 3 hours at R.T under nitrogen atmosphere, and then concentrated under reduced pressure to obtain a residue. The mixture of the residue and aqueous hydrochloric acid solution (1 M, 40 mL) was stirred overnight under nitrogen atmosphere at 100° C., cooled to room temperature, and then filtered. The filter cake was washed successively with water (30 mL) and Hex (300 mL), dried overnight under vacuum at 45° C. to afford INT A4-1 (5.03 g, 18.26 mmol). MS m/z: 273 [M−H]⁻.

DIPEA (12.26 g, 94.86 mmol) was added dropwise to a mixture of INT A4-1 (5 g, 18.15 mmol) and Phosphorus oxychloride (36 mL) at 0° C. The reaction mixture was stirred for 2.5 hrs at 100° C. under nitrogen atmosphere, and then concentrated under reduced pressure to obtain a residue. The residue was dissolved with DCM (600 mL) and washed with ice/water (400 mL), and the organic phase was concentrated under reduced pressure to obtain a crude product which was purified with silica gel chromatography to afford the INT A4 (1.6 g, 5.12 mmol).

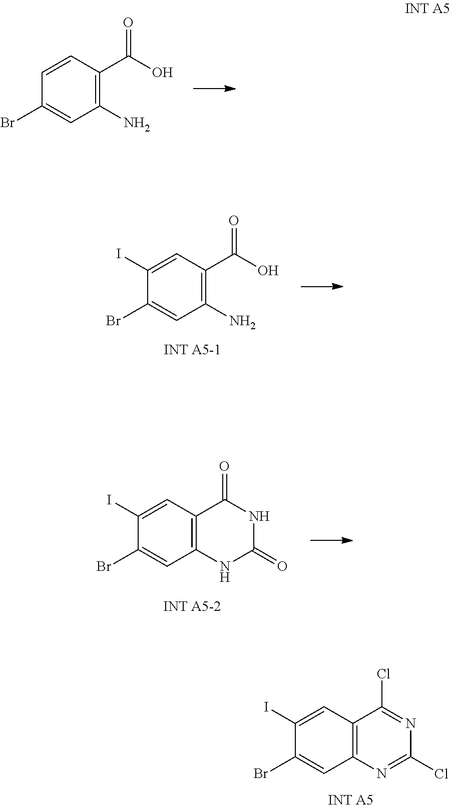

A solution of KI in water (3.93 g, 23.67 mmol) was added dropwise to a mixture of 2-amino-4-bromobenzoic acid (5.06 g, 23.42 mmol), NaCl (2.80 g, 47.91 mmol), NaIO₄ (5.00 g, 23.38 mmol) and AcOH (110 mL). The reaction mixture was stirred for 22 hrs at R.T, then poured into ice/water, diluted with saturated aq. Na₂S₂O₃, and extracted with DCM (3×100 mL). The organic layers were combined, washed with saturated aq. NaHCO₃, dried over Na₂SO₄ and concentrated under reduced pressure to obtain a residue which was purified with silica gel chromatography (eluting with EA) to afford INT A5-1 (6.87 g, yield 85.78%) as a brown solid. MS m/z: 340 [M−H]⁻.

Chlorosulfonyl isocyanate (6.92 g, 48.89 mmol) was added dropwise to a solution of INT A5-1 (6.87 g, 20.09 mmol) in DCM (30 mL) at 0° C. The reaction mixture was stirred for 7 hrs at R.T, and then concentrated under reduced pressure to obtain a residue. The mixture of the residue and aq. HCl (6 M, 50 mL) was stirred overnight at 100° C., cooled to R.T, poured into ice/water, and then filtered. The filter cake was collected and dried to afford INT A5-2 (5.38 g, 72.97%). MS m/z: 365 [M−H]⁻.

DIEA (4.89 g, 37.84 mmol) was added dropwise to a solution of INT A5-2 (5.38 g, 14.66 mmol) in POCl₃ (16 mL) at 0° C. The reaction mixture was stirred for 2 hrs at 110° C. under nitrogen atmosphere, and then concentrated under reduced pressure to obtain a residue. Ice-water (100 mL) was added slowly to the mixture of the residue and DCM (100 mL), and the organic layer was washed with brine (2×100 mL), dried over Na₂SO₄ and concentrated under reduced pressure to obtain a residue. The mixture of the residue and EA (400 mL) was washed with brine (3×200 mL), dried over Na₂SO₄ and concentrated under reduced pressure to afford the INT A5 (5.33 g, 13.20 mmol) as a yellow solid. MS m/z: 403/405 [M+H]⁺.

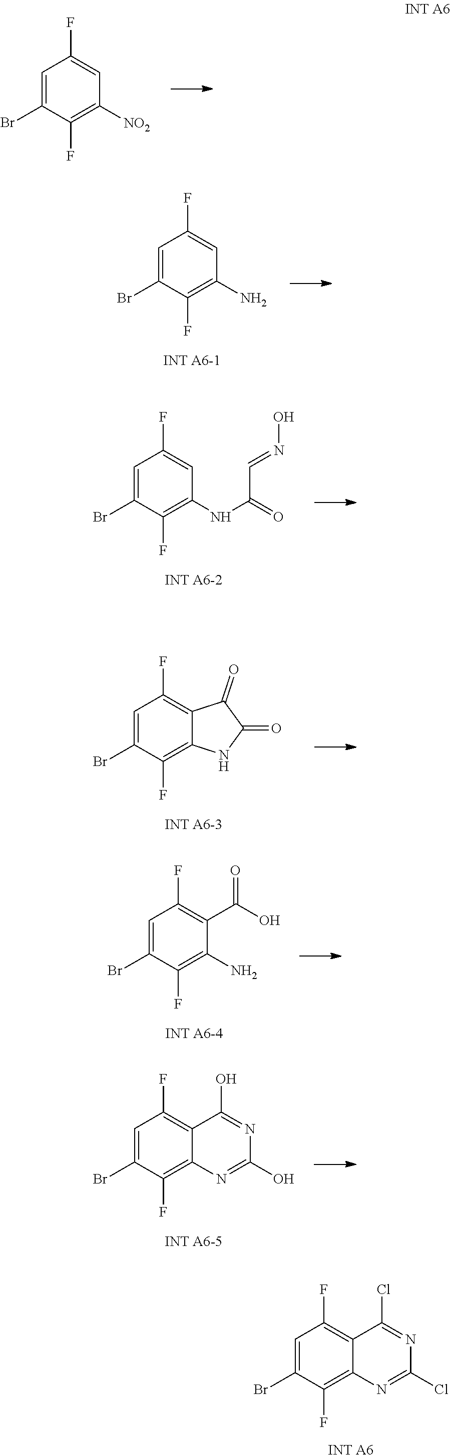

A mixture of 1-bromo-2,5-difluoro-3-nitrobenzene (3.11 g, 13.06 mmol), iron (2.12 g, 37.96 mmol), NH₄Cl (3.49 g, 65.24 mmol), ethanol (60 mL) and water (12 mL) was stirred at 80° C. for 2 hrs, and then filtered. The filtrate was concentrated under reduced pressure to obtain a residue. A solution of the residue in DCM (100 mL) was washed with saturated brine (2×30 mL), dried over anhydrous Na₂SO₄ and concentrated under reduced pressure to afford INT A6-1 (2.49 g, 11.97 mmol, 91.6% yield). MS (ESI, m/z): 208 [M+H]⁺.

Concentrated hydrochloric acid (1.75 mL) was added to a mixture of INT A6-1 (2.49 g, 11.97 mmol), hydroxylammoniumchlorid (2.49 g, 35.83 mmol), Na₂SO₄ (11.64 g, 95.76 mmol), chloral hydrate (2.56 g, 17.95 mmol), water (50 mL) and ethanol (7 mL). The reaction mixture was stirred at 60° C. for 16 hrs, cooled to R.T, and then filtered. The filter cake was dried under vacuum to afford INT A6-2 (3.295 g, 11.80 mmol, 98.6% yield). MS (ESI, m/z): 279 [M+H]⁺.

INT A6-2 (3.295 g, 11.80 mmol) was added to sulfuric acid (29.5 mL) at 60° C. The reaction mixture was stirred at 90° C. for 1 h, cooled to R.T, and added slowly to ice/water. The solid was collected by filtration, washed with water and dried under vacuum to afford INT A6-3 (2.173 g, 8.29 mmol, 70.2% yield). MS (ESI, m/z): 262[M+H]⁺.

Hydrogen dioxide (5.2 mL) was added dropwise to a solution of INT A6-3 (2.173 g, 8.29 mmol) in aq. NaOH (2 M, 46 mL, 93.50 mmol) at 0° C. The reaction mixture was stirred at R.T for 16 hrs, and then excessive sodium sulfite was added to quench excess hydrogen dioxide. After adjusting the pH to 7, the resulting mixture was filtered, and the pH of the filtrate was adjusted to 2 with concentrated hydrochloric acid. The solid was collected by filtration, washed with water and dried under vacuum to afford INT A6-4 (1.782 g, 7.07 mmol, 69.8% yield). MS (ESI, m/z): 252 [M+H]⁺.

Chlorosulfonyl isocyanate (1.33 g, 9.39 mmol) was added dropwise to a solution of INT A6-4 (1.782 g, 7.07 mmol) in dichloromethane (20 mL) at 0° C. The reaction mixture was stirred at R.T for 6 hrs and concentrated under reduced pressure to obtain a residue. A mixture of the residue and concentrated hydrochloric acid (20 mL) was stirred at 100° C. for 16 hrs, cooled to R.T, and then filtered. The filter cake was washed by water and dried under vacuum to afford INT A6-5 (0.83 g, 2.99 mmol, 75.5% yield). MS (ESI, m/z): 277 [M+H]⁺.

N,N-diisopropylethylamine (2 mL) was added to a solution of INT A6-5 (0.83 g, 2.99 mmol) in POCl₃ (15 mL). The reaction mixture was stirred at 105° C. for 2 hrs, and then concentrated under reduced pressure to obtain a residue. A solution of residue in DCM (50 mL) was washed by water (2×30 mL), dried over anhydrous Na₂SO₄, and then concentrated under reduced pressure to afford INT A6 (1.87 g, 5.95 mmol, 113.8% yield).

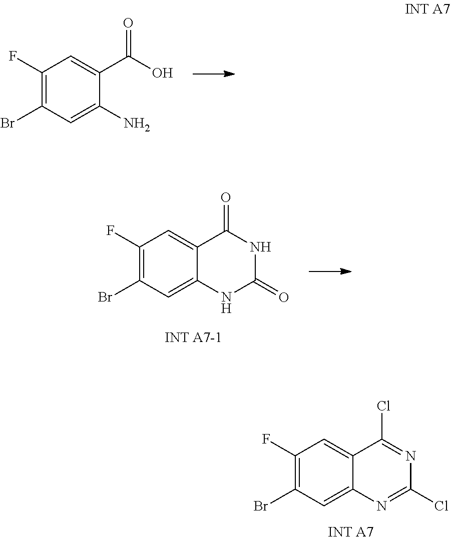

Chlorosulfonyl isocyanate (3.0 g, 21.00 mmol, 2.34 eq) was added dropwise over 15 mins to a solution of 2-amino-4-bromo-5-fluorobenzoic acid (2.1 g, 8.97 mmol, 1.0 eq) in DCM (35 mL) at 0° C. The reaction mixture was stirred for 48 hrs at R.T, and then concentrated to obtain a residue. A mixture of aq. hydrochloric acid (6 M, 70 mL) and the residue was stirred overnight at 105° C., cooled to R.T and then filtered to obtain a crude product containing INT A7-1 (1.15 g, 49.6% yield) as a white solid which was used for next step without any further purification. ¹H NMR (300 MHz, DMSO-d₆): δ 11.51 (s, 1H), 11.24 (s, 1H), 7.75 (d, J=8.4 Hz, 1H), 7.45 (d, J=5.7 Hz, 1H). LCMS: 257 ([M−H]⁻).

DIEA (8.7 g, 67.6 mmol, 5.0 eq) was added to a mixture of the crude product containing INT A7-1 (3.5 g, 13.5 mmol, 1.0 eq) and POCl₃ (35 mL) at R.T. The reaction mixture was concentrated after stirring overnight at 80° C., and then poured into ice/water. The solid was collected by filtration, and washed with water, dried to obtain a crude product containing INT A7 (2.7 g) as a yellow solid which was used for next step without any further purification. ¹H NMR (300 MHz, DMSO-d₆): δ 8.61 (d, J=6.6 Hz, 1H), 8.28 (d, J=8.4 Hz, 1H). LCMS: 295 ([M+H]⁺).

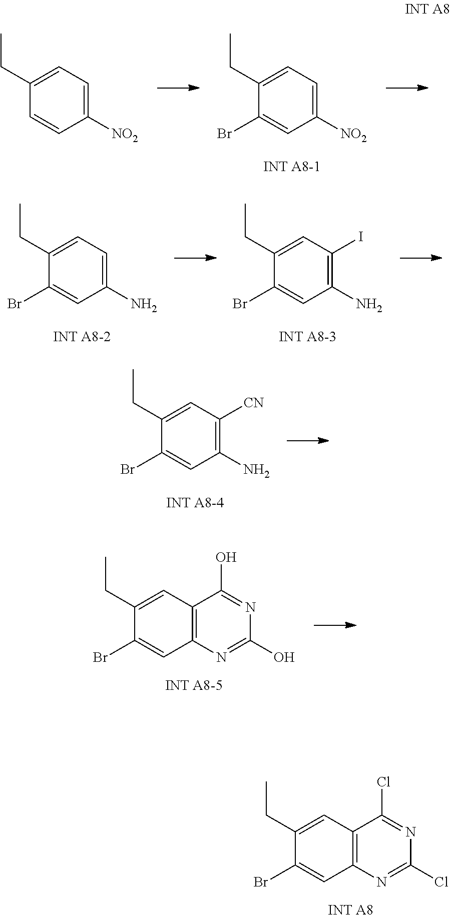

1-ethyl-4-nitrobenzene (10.0 g, 66 mmol, 1.0 eq), Ag₂SO₄ (21.0 g, 66 mmol, 1.0 eq), and Br₂ (11.0 g, 66 mmol, 1.0 eq) were added to a mixed solution of concentrated sulfuric acid (60 mL) and H₂O (7 mL). The reaction mixture was stirred at R.T for 1 h, and aq. sodium sulfite solution (10%, 100 mL) was added after the reaction was completed. The resulting mixture was extracted with EtOAc (100 mL×3). The organic phases were combined, dried over Na₂SO₄, and concentrated to afford a crude product containing INT A8-1 (16 g) as a brown solid which was used directly for next step without any further purification.

Fe powder (12.0 g, 217.3 mmol, 5.0 eq) was added to a solution of the crude product containing INT A8-1 (10.0 g) in a mixed solution of MeOH (25 mL) and AcOH (25 mL). The reaction mixture was stirred at R.T for 1 h, and then filtered after the reaction was completed. Ammonia water (105 mL) was added to the filtrate, and the resulting mixture was extracted with EtOAc (150 mL×3). The organic layers were combined, dried over Na₂SO₄, and concentrated to obtain a crude product containing INT A8-2 (9.8 g) as a brown solid which was used directly for next step without any further purification. LCMS: 200.0 ([M+H]⁺).

NIS (5.87 g, 26.0 mmol, 0.9 eq) was added to a solution of the crude product containing INT A8-2 (5.8 g) in AcOH (58 mL). The reaction mixture was stirred at R.T for 1 h, and then aq. Na₂SO₄ (1 M, 50 mL) was added. The resulting mixture was extracted with EtOAc (50 mL×3). The organic layers were combined, dried over Na₂SO₄ and then concentrated to obtain a residue which was purified with silica gel chromatography (petroleum ether/ethyl acetate=50:1) to afford INT A8-3 (3.7 g, 11.4 mmol, 39.3% yield) as a yellow solid. LCMS: 325.9 ([M+H]⁺).

Zn(CN)₂ (645.8 mg, 5.5 mmol, 0.5 eq) and Pd(pph₃)₄(638.1 mg, 0.55 mmol, 0.05 eq) were added to a solution of INT A8-3 (3.6 g, 11.0 mmol, 1.0 eq) in DMF (50 mL). The reaction mixture was stirred at 90° C. for 4.5 hrs under N₂ atmosphere, diluted with H₂O (60 mL) after the reaction was completed, and then extracted with EtOAc (60 mL×3). The organic layers were combined, dried over Na₂SO₄, and concentrated to obtain a crude product containing INT A8-4 (2.9 g) as a yellow solid which was used directly for next step without any further purification. LCMS: 225.0 ([M+H]⁺).

A solution of the crude product containing INT A8-4 (1.0 g) in DBU-HOCH₃CF₃ (10 ml) was stirred overnight at 25° C. under CO₂ atmosphere. The resulting mixture was diluted with H₂O (20 ml), and extracted with EtOAc (20 mL×3). The organic layers were combined, dried over Na₂SO₄ and concentrated to obtain a residue which was triturated and slurried with a mixed solution of DCM and MeOH (V_{DCM}/V_{MeOH}=10:1) to afford INT A8-5 (410 mg, 1.5 mmol, 38% yield) as a white solid. ¹H NMR (300 MHz, DMSO-d₆): δ 11.23 (s, 2H), 7.78 (s, 1H), 7.39 (s, 1H), 2.71 (q, J=7.4 Hz, 2H), 1.17 (t, J=7.5 Hz, 3H). LCMS: 269.0 ([M+H]⁺).

DIEA (0.75 ml) was dropwise added to a solution of INT A8-5 (410 mg, 1.5 mmol, 1.0 eq) in POCl₃ (7.5 mL). The reaction mixture was stirred at 107° C. for 2 hrs, and then concentrated to afford a crude product containing INT A8 as a black solid which was used directly for next step without any further purification. LCMS: 304.9 ([M+H]⁺).

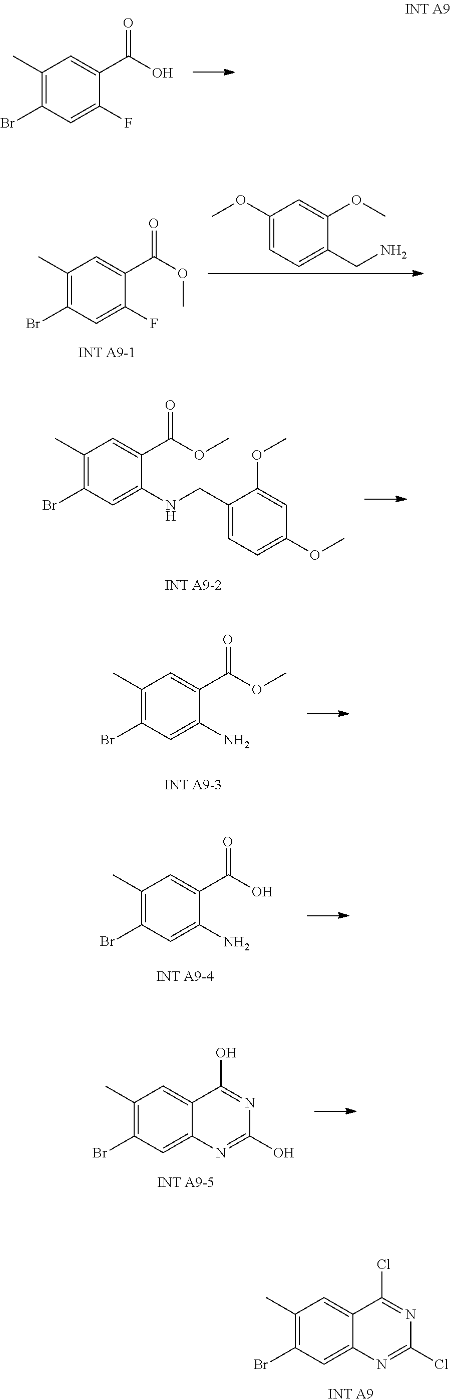

MeI (5.3 g, 37.5 mmol, 1.5 eq) and K₂CO₃ (10.4 g, 75.2 mmol, 3.0 eq) were added to a solution of 4-bromo-2-fluoro-5-methylbenzoic acid (5.8 g, 24.9 mmol, 1.0 eq) in DMF (60 mL) below 10° C. The reaction mixture was stirred for 30 mins at R.T, poured into water (100 mL) and extracted with EtOAc (100 mL×2). The organic layers were combined, washed with water (75 mL×2) and brine (50 mL×2) successively, dried over anhydrous Na₂SO₄, and concentrated to obtain a residue which was slurried with hexane and filtered to afford INT A9-1 (5.4 g, yield 83.3%) as a white solid. ¹H NMR (300 MHz, CDCl₃): δ 7.79 (d, J=7.4 Hz, 1H), 7.36 (d, J=9.9 Hz, 1H), 3.92 (s, 3H), 2.39 (s, 3H). LCMS: 247.0, 249.0 ([M+H]⁺).

2,4-Dimethoxybenzylamine (5.2 g, 30.6 mmol, 1.2 eq) and K₂CO₃ (10.6 g, 76.5 mmol, 3.0 eq) were added to a solution of INT A9-1 (6.3 g, 25.5 mmol, 1.0 eq) in 1,4-dioxane (60 mL) at R.T. The reaction mixture was stirred for 3 hrs at 100° C., cooled to room temperature, poured into ice-water (20 mL) and extracted with EtOAc (20 mL×3). The organic layers were combined, washed with brine (20 mL), dried with anhydrous Na₂SO₄, and concentrated to afford the residue containing INT A9-2 which was used for next step without further purification. ¹H NMR (300 MHz, DMSO-d₆): δ 7.83 (t, J=6.0 Hz, 1H), 7.65 (d, J=0.42, 1H), 7.15 (d, J=8.1 Hz, 1H), 6.99 (s, 1H), 6.61-6.59 (m, 1H), 6.51-6.47 (m, 1H), 4.28 (d, J=6.0 Hz, 2H), 3.83 (s, 3H), 3.79 (s, 3H), 3.75 (s, 3H), 2.21 (s, 3H).

A mixture of the residue containing INT A9-2 (7.4 g, 21.4 mmol, 1.0 eq), TFA (3 mL) and DCM (15 mL) was stirred at the R.T for 3 hrs, and then concentrated to afford a crude product containing INT A9-3 (5.4 g) which was used for next step without further purification. LCMS: 244.2, 246.2 ([M+H]⁺).

NaOH (1.9 g, 46.2 mmol, 3.0 eq) was added to a solution of the crude product containing INT A9-3 (4.6 g, 15.4 mmol, 1.0 eq) in a mixed solution of THF, EtOH, and water (V_{THF}/V_{EtOH}/V_{water}=3:1:1, 50 mL) at R.T. The reaction mixture was stirred for 2.5 hrs at 50° C., then concentrated, diluted with water (20 mL) and extracted with EtOAc (40 mL). The organic layer was washed with brine (20 mL), dried with anhydrous Na₂SO₄, and concentrated to obtain a residue which was purified with silica gel chromatography (eluting with petroleum ether/EtOAc=30:1) to afford a crude product containing INT A9-4 (5.2 g) as a white solid which was used for next step without further purification. LCMS: 227.8, 230.0 ([M−H]⁻).

Chlorosulfonyl isocyanate (7.3 g, 51.6 mmol, 2.4 eq) was added dropwise to a solution of the crude product containing INT A9-4 (5.0 g, 21.5 mmol, 1.0 eq) in DCM (50 mL). The reaction mixture was stirred at room temperature for 1 h, and then concentrated to obtain a residue. A mixture of the residue and aq. hydrochloric acid (6 M, 100 mL) was stirred for 16 hrs at 100° C., cooled to R.T and filtered to afford a solid containing INT A9-5 (4.5 g, yield 82.0%) as a white solid which was used for next step without further purification. ¹H NMR (300 MHz, DMSO-d₆): δ 11.33 (brs, 1H), 11.12 (brs, 1H), 7.83 (s, 1H), 7.38 (s, 1H), 2.36 (s, 3H). LCMS: 252.8, 254.8 ([M−H]⁻).

DIEA (646.0 mg, 5.0 mmol, 5.0 eq) was added to a solution of the solid containing INT A9-5 (1.0 g, 3.9 mmol, 1.0 eq) in POCl₃ (20 mL) at R.T The reaction mixture was heated to reflux and stirred for 16 hrs, and then concentrated under reduced pressure to obtain a residue which was purified with silica gel chromatography (eluting with petroleum ether/EtOAc=100:1) to afford INT A9 (400.0 mg, yield 35.1%) as a white solid. LCMS: 292.9 ([M+H]⁺).

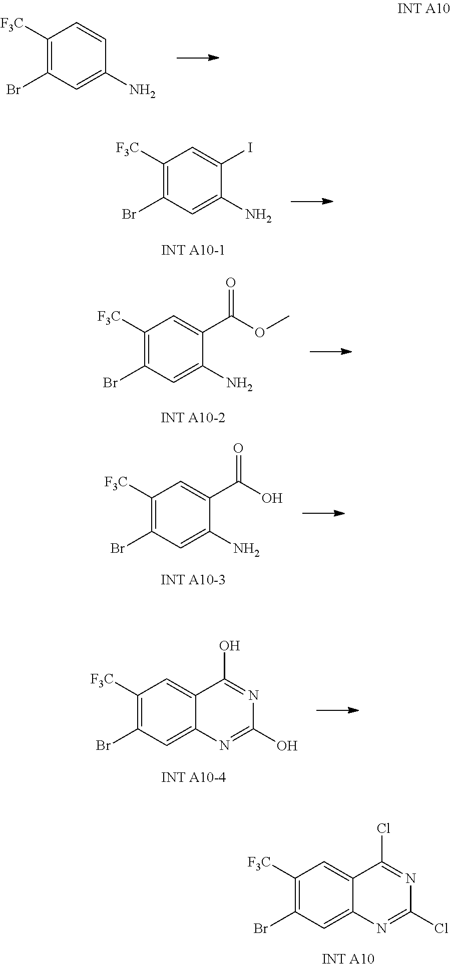

NIS (5.6 g, 25.0 mmol, 1.0 eq) was added in batches to a solution of 3-bromo-4-(trifluoromethyl)aniline (6.0 g, 25.0 mmol, 1.0 eq) in AcOH (60 mL) while the temperature was below 10° C. The reaction mixture was stirred at R.T for 5 hrs, and then concentrated to obtain a residue which was purified with silica gel chromatography (eluting with petroleum ether) to afford INT A10-1 (7.0 g, yield 85.9%) as a white solid. ¹H NMR (300 MHz, DMSO-d₆): δ 7.82 (s, 1H), 7.07 (s, 1H), 6.19 (s, 2H). LCMS: 363.8, 365.8 ([M−H]⁻).

A 300 mL stainless steel autoclave was charged with INT A10-1 (3.1 g, 8.5 mmol, 1.0 eq), Pd(dppf)Cl₂-DCM (694 mg, 0.85 mmol, 0.1 eq), TEA (6.0 g, 59.5 mmol, 7.0 eq) and MeOH (30 mL). The autoclave was sealed and stirred at 30° C. under 0.8 MPa of carbon monoxide for 13 h. After the reaction was completed, the resulting mixture was concentrated to obtain a residue which was purified with silica gel chromatography (eluting with petroleum ether/EtOAc=100:1) to afford INT A10-2 (1.8 g, yield 71.1%) as a white solid. ¹H NMR (300 MHz, CDCl₃): δ 8.15 (s, 1H), 6.99 (s, 1H), 6.11 (brs, 2H), 3.89 (s, 3H). LCMS: 297.8, 299.8 ([M+H]⁺).

NaOH (720.0 mg, 18.0 mmol, 3.0 eq) was added to a solution of INT A10-2 (1.8 g, 6.0 mmol, 1.0 eq) in a mixed solution of THF, EtOH, and water (V_{THF}/V_{EtOH}/V_{water}=3:1:1, 40 mL) at room temperature. The reaction mixture was stirred for 3 hrs at 50° C., and then diluted with water (20 mL). The pH of the resulting mixture was adjusted to 2 with 6 M HCl, and then EtOAc (40 mL) was added. The organic layer was washed with brine (20 mL), dried with anhydrous Na₂SO₄, and concentrated to afford a crude product containing INT A10-3 (1.6 g, 93.0%) which was used for next step without further purification. LCMS: 282.0, 283.9 ([M−H]⁻).

Chlorosulfonyl isocyanate (1.9 g, 13.5 mmol, 2.4 eq) was added dropwise to a solution of the crude product containing INT A10-3 (1.6 g) in DCM (20 mL). The reaction mixture was stirred at R.T for 2 hrs, and then concentrated to obtain a residue. A mixture of the residue and aq. HCl (6 M, 100 mL) was stirred for 16 hrs at 100° C., cooled to R.T and filtered to afford a crude product containing INT A10-4 (1.2 g, 69.0%) as a white solid which was used for next step without further purification. LCMS: 306.8, 308.8 ([M−H]⁻).

DIEA (1.9 g, 15.0 mmol, 5.0 eq) was added to a mixture of the crude product containing INT A10-4 (900.0 mg, 3.0 mmol, 1.0 eq) and POCl₃ (20 mL) at R.T. The reaction mixture was heated to refluxed and stirred for 16 hrs, and then concentrated under reduced pressure to obtain a residue which was purified with silica gel chromatography (eluting with petroleum ether/EtOAc=100:1) to afford a crude product containing INT A10 (330 mg) as a yellow solid which was used for next step without further purification.

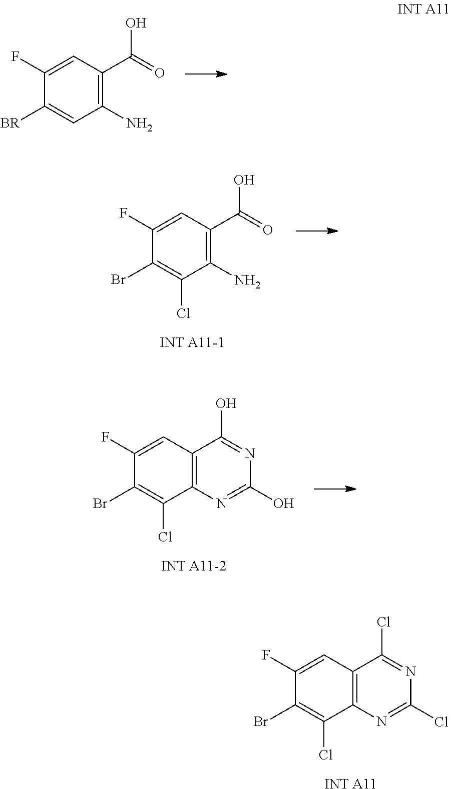

NCS (1.7 g, 13.0 mmol, 1.0 eq) was added in batches to a solution of 2-amino-4-bromo-5-fluorobenzoic acid (3.1 g, 13.0 mmol, 1.0 eq) in DMF (124.0 mL) at R.T. The reaction mixture was stirred overnight, diluted with water (300 mL) after the reaction was completed, extracted with EtOAc (3×300 mL). The organic layers were combined, washed with brine and dried over anhydrous Na₂SO₄, concentrated to afford a crude product containing INT A11-1 (2.4 g) which was used directly to the next step without further purification. ¹H NMR (400 MHz, DMSO-d₆) δ 7.64 (d, J=9.4 Hz, 1H). LCMS: 265.9 ([M−H]⁻).

ClSO₂NCO (2.5 g, 17.5 mmol, 2.3 eq) was added slowly to a solution of the crude product containing INT A11-1 (2.0 g) in DCM (46 mL) at 0° C. The reaction mixture was stirred at R.T for 4 hrs, and then concentrated to afford a residue. A mixture of the residue and aq. HCl (6 M, 54 mL) was heated to reflux, stirred overnight, cooled to precipitate the solid. The solid was collected by filtration, dried to afford a crude product containing INT A11-2 (2.1 g) as a yellow solid which was used directly to the next step without further purification. ¹H NMR (300 MHz, DMSO-d₆) δ 11.72 (s, 1H), 10.87 (s, 1H), 7.78 (d, J=8.0 Hz, 1H). LCMS: 290.9 ([M−H]⁻).

DIEA (1.7 mL) was added to a mixture of the crude product containing INT A11-2 (1.6 g) and POCl₃ (17 mL) at R.T. The reaction mixture was stirred at 110° C. for 5 hrs, and then concentrated to afford a crude product containing INT A11 which was used directly to the next step without any further purification.

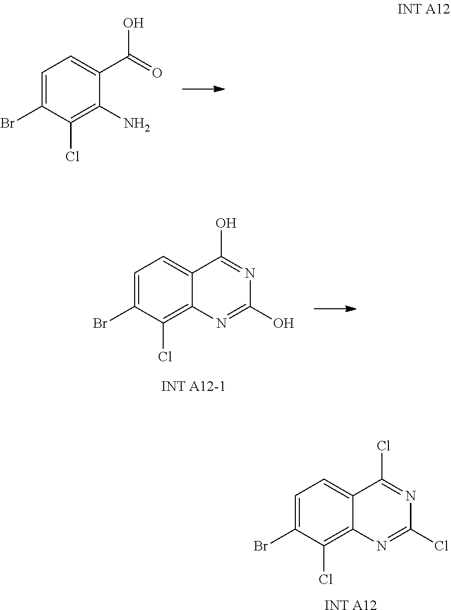

INT A12 was synthesized following the procedure of INT A3 with 2-amino-4-bromo-3-chlorobenzoic acid as starting material.

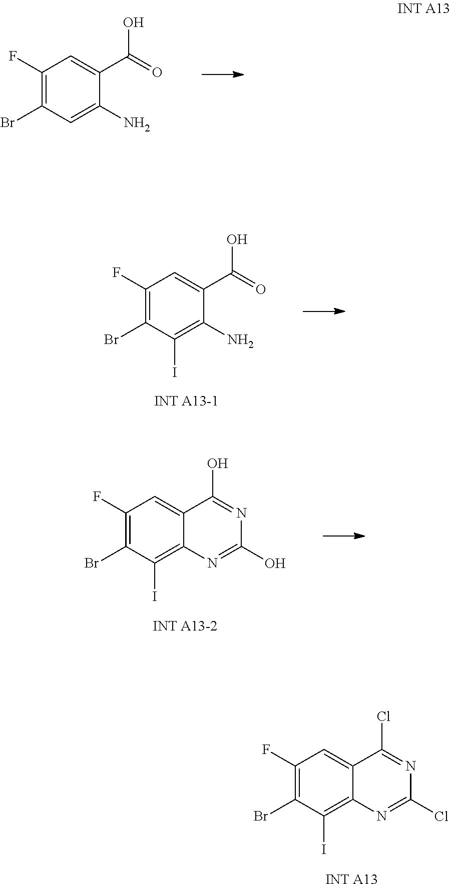

INT A13 was synthesized following the procedure of INT A2 with 2-amino-4-bromo-5-fluorobenzoic acid as starting material.

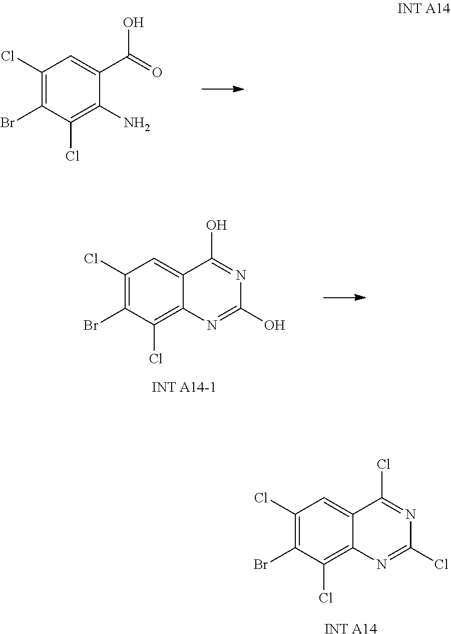

INT A14 was synthesized following the procedure of INT A1 with 2-amino-4-bromo-3,5-dichlorobenzoic acid as starting material.

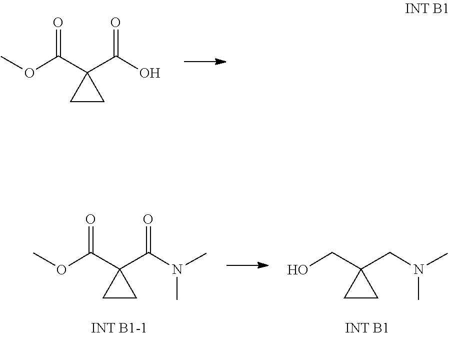

Oxalyl chloride (22.65 g, 178.45 mmol) was added to a mixed solution of 1-(methoxycarbonyl)cyclopropane-1-carboxylic acid (20.06 g, 139.18 mmol) in DCM (100 mL) and DMF (100 mg, 1.37 mmol) at 0° C. under nitrogen atmosphere. The solution stirred for about 3 h at room temperature. The solution was concentrated to a yellow semi-solid.

The semi-solid was taken up in THF (50 mL) and the solution cooled to 0° C., and dimethylamine/THF (60 mL, 120 mmol, 2M) was added dropwise slowly and the resulting suspension stirred for about 2 h. EA (200 mL) was added and the mixture washed with brine (2×200 mL). The organic layer was dried over anhydrous Na₂SO₄ and concentrated to yield INT B1-1 (7.37 g, 43.05 mmol) as yellow/brown oil. MS m/z: 172 [M+H]⁺.

Lithium aluminium hydride (3.21 g, 84.59 mmol) was added portion-wise to a solution of INT B1-1(7.37 g, 43.05 mmol) in THF at 0° C., and the resulting solution stirred for 2 h at room temperature. The solution was cooled to 0° C., water (3.5 mL), NaOH solution (15%, 3.5 mL), water (10 mL) was added portion-wise to afford a white suspension. The resulting suspension was filtered through Celite and washed with THF (150 mL). The combined filtrates were concentrated and purified by silica gel chromatography (eluting with DCM:MeOH=1:10, v/v) to afford INT B1 (3.79 g, 29.33 mmol) as pale yellow oil. MS m/z: 130 [M+H]⁺.

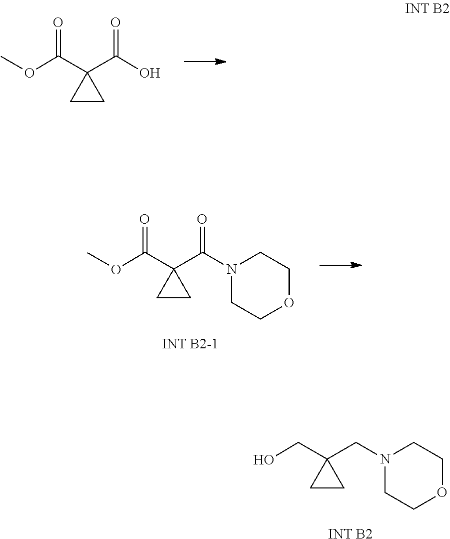

Oxalyl chloride (53.55 g, 0.42 mmol) was added to a mixed solution of 1-(methoxycarbonyl)cyclopropane-1-carboxylic acid (50.45 g, 350.04 mmol) in DCM (500 mL) and DMF (5.16 g, 70.59 mmol) at 0° C. under nitrogen atmosphere. The solution stirred for about 3 h at room temperature. The solution was concentrated to a yellow semi-solid.

The semi-solid was taken up in DCM (100 mL) and dropped to a solution of morpholine (34.00 g, 390.27 mmol) and TEA (52.93 g, 523.08 mmol) in DCM (500 mL) at 0° C. The resulting suspension was stirred for 18 h. Water (500 mL) was added and the mixture was extracted with EA. The organic layer was washed with aq. citric acid (5%, 500 mL) followed by brine (500 mL), then dried over anhydrous Na₂SO₄ and concentrated under reduced pressure. The residue was purified by silica gel chromatography (eluted with EA:Hex) to afford INT B2-1(70.05 g, 328.52 mmol, 93.85% yield) as yellow/brown oil. MS m/z: 214 [M+H]⁺.

Lithium aluminium hydride (30 g, 790.51 mmol) was added portion-wise to a solution of INT B2-1 (70.05 g, 328.52 mmol) in THF (700 mL) at 0° C., and the resulting solution was stirred for 3 h at room temperature. The solution was cooled to 0° C., water (30 mL), NaOH solution (15%, 30 mL), water (90 mL) was added portion-wise to afford a white suspension. The resulting suspension was filtered through Celite, and the solids were washed with THF (150 mL). The combined filtrates were concentrated and purified by silica gel chromatography (eluting with DCM:MeOH=1:50, v/v) to afford INT B2 (40.61 g, 237.16 mmol, 72.19 yield) as colorless oil. MS m/z: 172 [M+H]⁺.

The following compounds were synthesized using above procedure with corresponding starting material.

| Name | Structure |
|---|---|
| INT B3 | 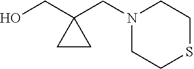 [SMILES: OCC1(CN2CCSCC2)CC1] |
| INT B4 | 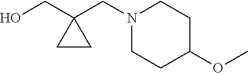 |
| INT B5 | 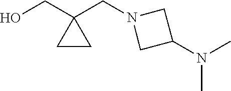 |
| INT B6 | 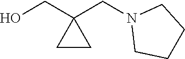 [SMILES: OCC1(CN2CCCC2)CC1] |
| INT B7 | 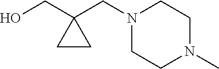 |
| INT B8 | 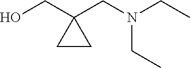 |
| INT B9 | 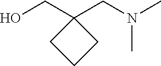 |
| INT B10 | 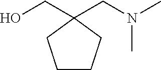 |
| INT B11 | 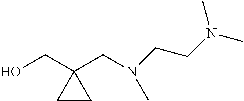 |

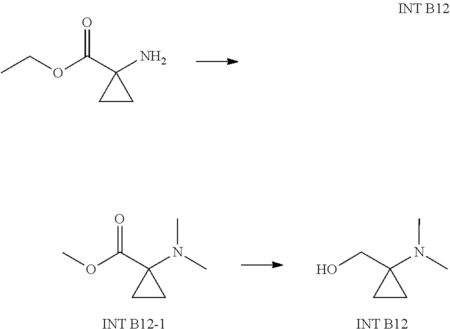

To a solution of ethyl 1-aminocyclopropane-1-carboxylate (529 mg, 3.19 mmol) in formaldehyde (5 mL) and methanol (50 mL) was added Pd/C (0.26 g, 10% wt) at hydrogen atmosphere. The reaction mixture was stirred at room temperature for 15 h. The resulting mixture was filtered and the filtrate was concentrated under reduced pressure. The residue was dissolved in DCM (50 mL), extracted with NaHCO₃ (sat., 3×20 mL), dried over anhydrous Na₂SO₄ and concentrated under reduced pressure to afford INT B12-1 (787 mg, 5.50 mmol) as yellow oil. MS (ESI, m/z):158 [M+H]⁺.

To a solution of INT B12-1 (397 mg, 2.77 mmol) in THF (10 mL) was slowly added hydrogen lithium aluminium (0.18 g, 4.74 mmol) at 0° C. under nitrogen atmosphere. The reaction mixture was stirred at room temperature for 2 h. Then the resulting mixture was quenched by water (0.2 mL), followed by aq. 15% NaOH (0.2 mL), water (0.6 mL) and filtered, the filtrate was concentrated under reduced pressure. The residue was purified by silica gel chromatography (eluted with 0-10% methanol in DCM) to afford INT B12 (254 mg, 2.21 mmol) as colorless oil. MS (ESI, m/z):116 [M+H]⁺.

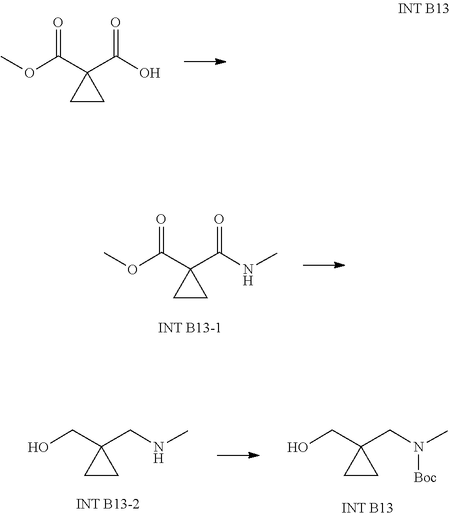

To a solution of 1-(methoxycarbonyl) cyclopropane-1-carboxylic acid (2.25 g, 15.61 mmol), DMF (2 drops) in DCM (30 mL) was added oxalyl dichloride (2.64 g, 20.80 mmol) dropwise. After the solution was stirred for 2.5 h at room temperature, the solution was concentrated in vacuum. The residue was dissolved in DCM (8 mL) to afford the solution A.

A solution of methylamine alcohol solution (5.39 g, 33% wt, 57.27 mmol) in DCM (10 mL) was cooled to −70° C. To the solution was added the solution A dropwise while maintaining the temperature at −75° C. to −70° C. for 0.5 h. The reaction solution was diluted with H₂O (40 mL) and extracted with EA (2×40 mL). The combined extracts were washed with brine, dried over Na₂SO₄, filtered and concentrated in vacuum. The residue was purified by silica gel chromatography (10-40% Hex/EA) to afford INT B13-1 (2.34 g). MS m/z 158 [M+H]⁺.

To a solution of INT B13-1 (2.34 g, 14.89 mmol) in THF (25 mL) was added LAH (1.2 g, 31.62 mmol) in portions. The mixture was heated to 70° C. for 3 h. The mixture was quenched with H₂O (1.5 mL), 15% sodium hydroxide solution (3 mL) and H₂O (5 mL). The suspension was filtered and concentrated. The residue was purified by silica gel chromatography (eluted with 10-40% Hex/EA) to afford INT B13-2 (1.48 g). MS m/z 116 [M+H]⁺.

To a solution of INT B13-2 (1.48 g, 12.85 mmol), TEA (1.30 g, 12.85 mmol) in DCM (15 mL) at −10° C. was added di-tert-butyl dicarbonate (2.80 g, 12.83 mmol). The solution was stirred for 3 h. The solution was concentrated in vacuum and purified by silica gel chromatography (eluted with 10-30% Hex/EA) to afford INT B13 (1.95 g). MS m/z 216 [M+H]⁺.

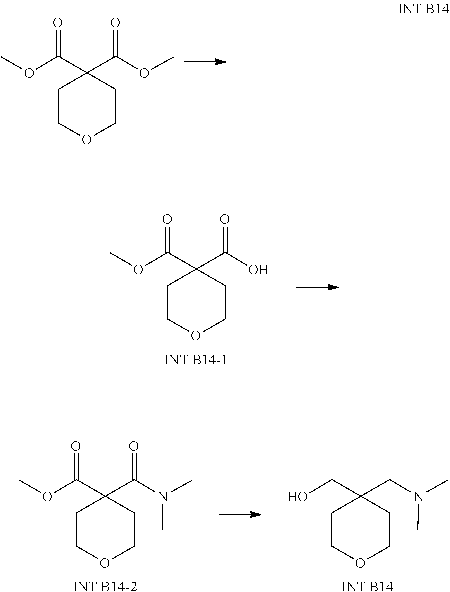

To a solution of dimethyl tetrahydro-4H-pyran-4,4-dicarboxylate (9.0 g, 44.5 mmol) in THF (45.0 mL), MeOH (45 mL) and water (45 mL) was added to LiOH (3.7 g, 89 mmol). The mixture was stirred at room temperature for 0.5 h. After completion, the solvent was evaporated and the residue was neutralized with 1 M HCl (20 mL) at 0° C. The mixture was extracted with EtOAc (50 mL×3), and brine (30 mL×3), dried over Na₂SO₄ and concentrated to afford INT B14-1(7.5 g, 90.3%) as brown oil. MS (ESI, m/z): 189 [M+H]⁺. ¹H NMR (300 MHz, DMSO-d₆): δ 13.17 (s, 1H), 3.68 (s, 3H), 3.57-3.53 (m, 4H), 1.95-1.92 (m, 4H).

INT B14 was synthesized following the procedure of INT B2 with INT B14-1 as starting material.

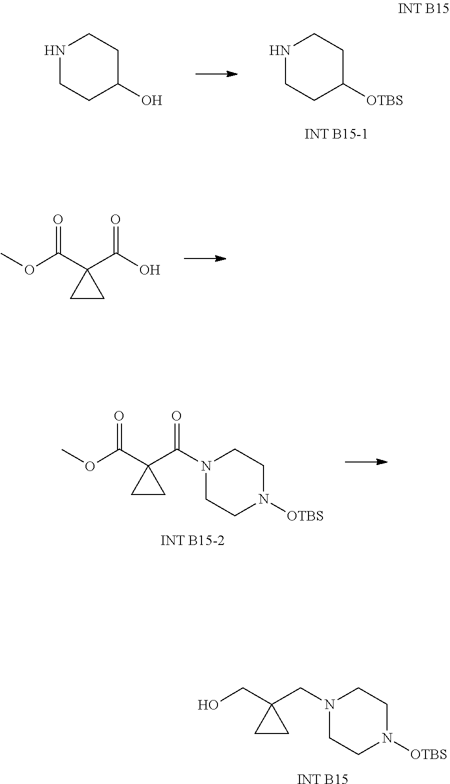

To a solution of piperidin-4-ol (10 g, 0.1 mol) and Imidazole (20.4 g, 0.3 mol) in DCM (100 mL) was added a solution of TBSCl (18.1 g) in DCM (20 mL). The mixture was stirred at 25° C. for overnight. Then water (100 mL) was added and extracted with EtOAc (50 mL×3) and brine (50 mL×3), dried over Na₂SO₄. The combined organic layers were concentrated under vacuum. The residue was purified by silica gel column chromatography (Petroleum ether/EtOAc=50:1, v/v) to afford INT B15-1 (17 g, 80.1%) as a white solid. ¹H NMR (400 MHz, CDCl₃): 9.00 (brs, 1H), 4.03 (s, 1H), 3.26 (td, J=2.8, 12.0 Hz, 2H), 3.14 (dt, J=3.5, 8.6 Hz, 2H), 2.08 (t, J=11.5 Hz, 2H), 1.70 (dd, J=3.5, 14.1 Hz, 2H), 0.83 (s, 9H), 0.01 (s, 6H).

To a solution of 1-(methoxycarbonyl) cyclopropane-1-carboxylic acid (500 mg, 3.5 mmol) in DCM (10 mL) was added INT B15-1 (1.1 g, 5.2 mmol), HATU (2.0 g, 5.2 mmol) and DIPEA (1.6 g, 12.1 mmol). The mixture was stirred at 20° C. for 16 h. After completion, the mixture was quenched with water (10 mL) and extracted with EtOAc (10 mL×3) and brine (10 mL×3), dried over Na₂SO₄, concentrated to afford INT B15-2 (800 mg, 67.5%) as colorless oil which used without any further purification. MS (ESI, m/z): 342 [M+H]⁺.

To a solution of INT B15-2 (800 mg, 2.3 mmol) in THF (8.0 mL) was added LAH (152 mg, 4.6 mmol) at 0° C. The mixture was stirred at 25° C. for 16 h. After completion, the reaction mixture was quenched with H₂O (2.0 mL), 15% aqueous NaOH (2.0 mL) and H₂O (2 mL). Then the slurry was filtered and the filtrate was concentrated to afford the crude, which was purified by C18 column (MeCN:H₂O=40:60) to afford INT B15 (500 mg, 70.4%) as colorless oil. MS (ESI, m/z): 300 [M+H]⁺. ¹H NMR (300 MHz, CDCl₃): 3.72 (brs, 1H), 3.52 (s, 2H), 2.80 (brs, 2H), 2.44 (s, 2H), 2.33 (s, 2H), 1.78-1.71 (m, 2H), 1.62-1.54 (m, 2H), 0.87 (s, 9H), 0.48 (t, J=6.0 Hz, 2H), 0.33 (t, J=6.0 Hz, 2H), 0.03 (s, 6H).

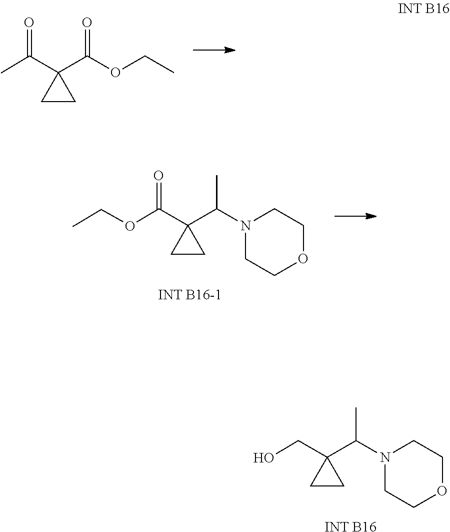

Sodium triacetoxyborohydride (8.43 g, 39.97 mmol) was added to a solution of ethyl 1-acetylcyclopropane-1-carboxylate (1.98 g, 12.68 mmol) and morpholine (1.76 g, 20.20 mmol) in ethylene chloride (40 mL). The reaction mixture was stirred at 55° C. for 16 hrs, diluted with water (40 mL) and extracted with DCM (5×30 mL). The organic phase were combined and concentrated under reduced pressure to obtain a residue which was purified with silica gel chromatography to afford INT B16-1 (216 mg, 0.95 mmol). MS m/z: 228 [M+H]⁺.

LAH (70 mg, 1.85 mmol) was added to a solution of INT B16-1 (210 mg, 0.92 mmol) in THF (10 mL). The reaction mixture was stirred at R.T for 3 hrs, and then water (0.08 mL), NaOH (0.08 mL, 15% w/w in water) and water (0.24 mL) were successively added dropwise. The mixture was filtered, and the filtrate was concentrated under reduced pressure to obtain a residue which was purified with silica gel chromatography to afford INT B16 (128 mg, 0.69 mmol). MS m/z: 186 [M+H]⁺.

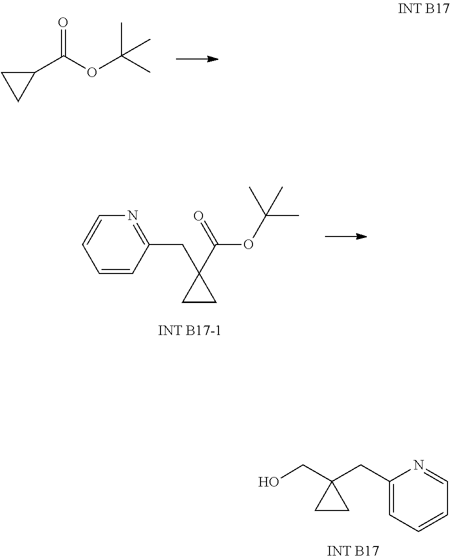

A solution of tert-butyl cyclopropanecarboxylate (5.0 g, 35.2 mmol, 1.0 eq) in THF (120 mL) was purged with nitrogen for 10 mins, and then LDA (2.0 M, 19 mL, 1.1 eq) was added dropwise at −78° C. under nitrogen atmosphere. After stirring for 1 h at −78° C., a solution of 2-(bromomethyl) pyridine (6.1 g, 56.3 mmol, 1.6 eq) in THF (30 mL) was added dropwise to the reaction mixture at −78° C. The resulting mixture was warmed to R.T, stirred for 16 hrs, quenched with saturated NH₄Cl (50 mL), diluted with water (100 mL) and extracted with EtOAc (100 mL×2). The organic layers were combined, washed with brine (100 mL), dried over Na₂SO₄, and then filtered. The filtrate was concentrated to obtain a residue which was purified with silica gel chromatography (eluting with petroleum ether/EtOAc=10:1) to afford INT B17-1 (2.2 g) as a white solid. LCMS: 234.2 ([M+H]⁺).

LAH (1.8 g, 47.1 mmol, 5.0 eq) was added to a solution of INT B17-1 (2.2 g, 9.4 mmol, 1.0 eq) in THF (20 mL) at 0° C. The reaction mixture was stirred at R.T for 2 hrs, cooled to 0° C. and Na₂SO₄·10H₂O (4.5 g, 14.1 mmol, 1.5 eq) was added in batches. The resulting mixture was stirred at R.T for 2 hrs and then filtered. The filtrate was concentrated to obtain a residue which was purified with silica gel chromatography (eluting with petroleum ether/EtOAc=2:1) to afford INT B17 (800.0 mg, the yield of the above two steps is 14.0%) as yellow oil. ¹H NMR (300 MHz, CDCl₃): δ 8.52-8.50 (m, 1H), 7.61 (td, J=7.5, 1.8 Hz, 1H), 7.18-7.10 (m, 2H), 3.44 (s, 2H), 2.94 (s, 2H), 0.56-0.53 (m, 2H), 0.46-0.44 (m 2H). LCMS: 164.20 ([M+H]⁺).

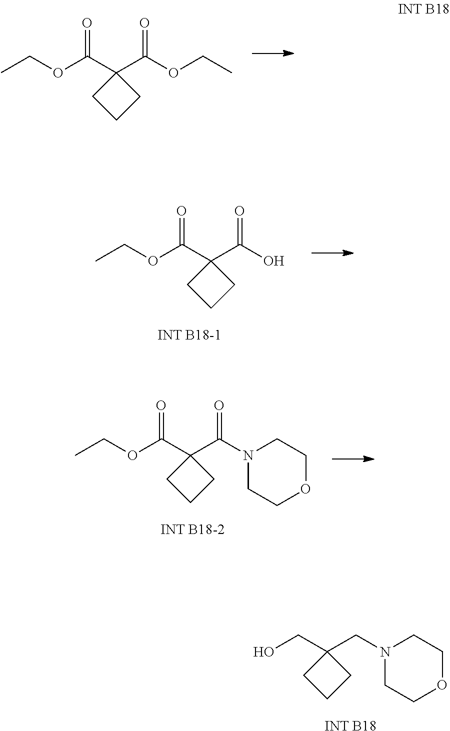

Aq. KOH (1.8 M, 41.6 ml, 75.0 mmol, 1.0 eq) was added dropwise to a solution of diethyl cyclobutane-1,1-dicarboxylate (15.0 g, 75.0 mmol, 1.0 eq) in EtOH (75 mL). After stirring at R.T for 4 hrs, the pH of the resulting mixture was adjusted to 2 with aq. HCl (2 M) and then extracted with EtOAc (60 mL×2). The organic layers were combined, dried over Na₂SO₄ and concentrated to afford a crude product containing INT B18-1 (13.5 g) as yellow oil. LCMS: m/z=173.20 [M+H]⁺.

Oxalyl Chloride (14.6 g, 115.0 mmol, 1.5 eq) was added dropwise to a solution of INT B18-1 (13.2 g, 76.7 mmol, 1.0 eq) in DCM (130 mL) at 0° C., and then DMF (0.5 mL) was added. The reaction mixture was stirred at R.T for 4 hrs, and then concentrated to obtain a residue after the HPLC analysis showed the reaction was completed. The Morpholine (10.0 g, 115.0 mmol, 1.5 eq) was added dropwise to a solution of the residue in THF (130 mL) at 0° C. The reaction mixture was stirred at R.T for 5 hrs, poured into water (100 mL) and extracted with EtOAc (100 mL). The organic layer was separated, washed with brine (100 mL), dried with anhydrous Na₂SO₄, and concentrated to obtain a residue which was purified with silica gel chromatography (eluting with petroleum ether/EtOAc=5:1) to afford INT B18-2 (7.2 g, yield 35.7%) as yellow oil. LCMS: m/z=242.20 [M+H]⁺.

LAH (945 mg, 32.7 mmol, 2.0 eq) was added in batches to a solution of INT B18-2 (3.5 g, 16.3 mmol, 1.0 eq) in THF (35 mL) at 0° C. The reaction mixture was stirred at R.T for 4 hrs, and then quenched with Na₂SO₄·10H₂O. The resulting mixture was stirred for 2 hrs, and then filtered. The filtrate was concentrated to obtain a residue which was purified with silica gel chromatography (eluting with petroleum ether/EtOAc=5:1) to afford INT B18 (1.2 g, yield 42.1%) as yellow oil. ¹H NMR (300 MHz, CDCl₃): δ 3.72 (s, 2H), 3.61-3.59 (m, 4H), 2.47 (s, 2H), 2.45-2.42 (m, 4H), 2.02-1.75 (m, 6H). LCMS: m/z=186 [M+H]⁺.

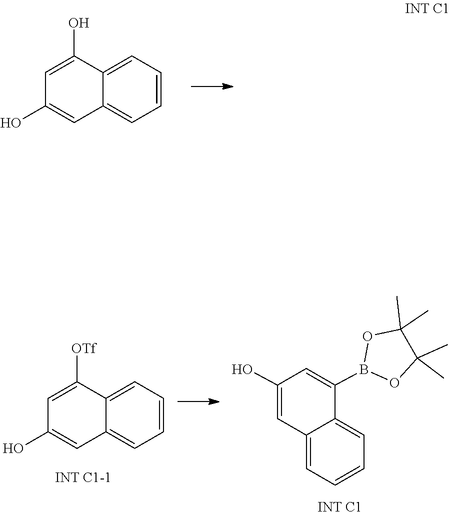

Trifluoromethanesulfonic anhydride (15.55 g, 55.12 mmol) and DIEA (7.37 g, 57.03 mmol) were added dropwise simultaneously to a solution of 1,3-dihydroxynaphthalene (8.82 g, 55.07 mmol) in DCM (300 mL) at 10° C. After stirred for 1 h, the solution was partitioned between water and DCM. The organic phase was dried over anhydrous Na₂SO₄, filtered and concentrated under vacuum. The residue was purified by silica gel chromatography (eluting with hexane/EA) to afford INT C1-1 (6.92 g, 23.68 mmol).

To a solution of INT C1-1 (6.87 g, 23.51 mmol) and 4,4,4′,4′,5,5,5′,5′-octamethyl-2,2′-bi(1,3,2-dioxaborolane) (11.66 g, 45.92 mmol) in 1.4-dioxane (100 mL) was added KOAc (8.39 g, 85.49 mmol) and Pd(dppf)Cl₂ (1.73 g, 2.36 mmol). The reaction mixture was stirred at 85° C. for 4.5 hours under nitrogen atmosphere. The reaction mixture was diluted with water (100 mL) and extracted with EA (150 mL, 100 mL). The combined organic layers were washed with brine (150 mL), dried over anhydrous Na₂SO₄ and concentrated under vacuum. The residue was purified by silica gel chromatography (eluting with Hex:EA=30:1˜15:1, v/v) to afford INT C1 (7.14 g, 26.43 mmol). MS m/z: 271 [M+H]⁺.

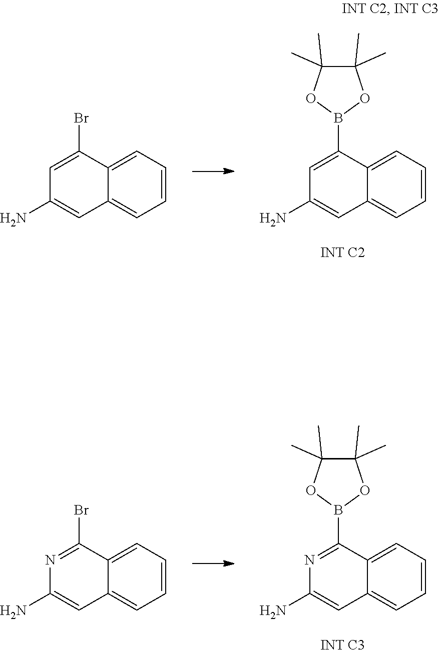

Following the procedure of INT C1, INT C2 and INT C3 were synthesized with corresponding starting materials.

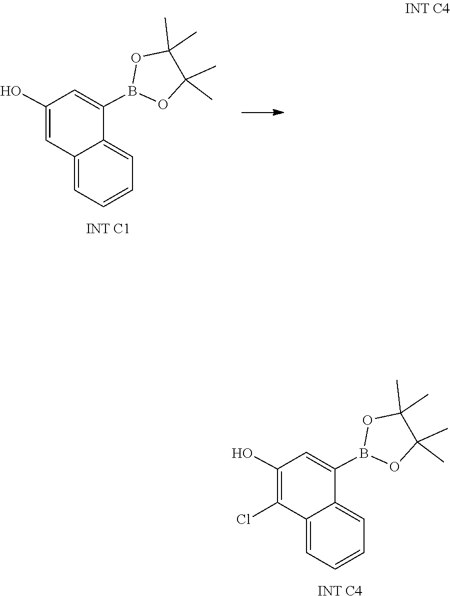

To a solution of INT C1 (200 mg, 0.74 mmol) in CHCl₃ (2 mL) was added NCS (82 mg, 0.74 mmol) and 1 drop DMSO. The mixture was stirred at 25° C. for 30 mins. After completion, water (5 mL) was added, and extracted with DCM (10 mL×3) and brine (10 mL×3), dried over Na₂SO₄. The organic layers were concentrated under vacuum. The residue was purified by silica gel column chromatography (Petroleum ether/EtOAc=5:1, v/v) to afford INT C4 (100 mg, 44.4%) as a white solid.

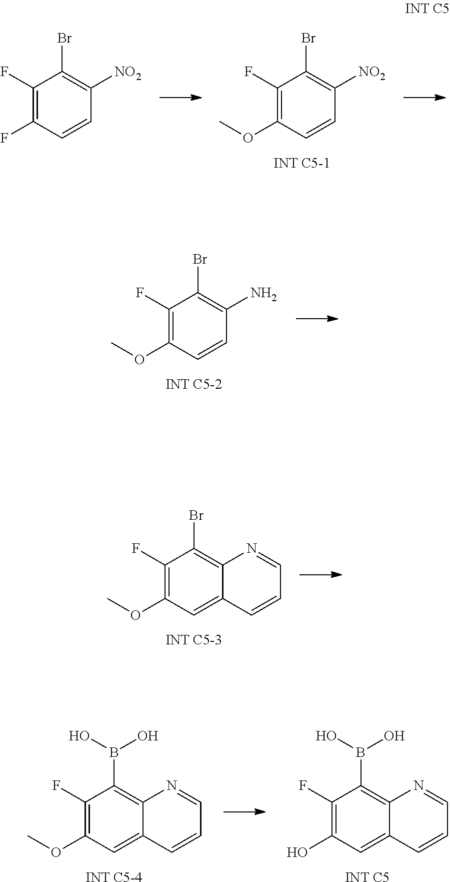

To a solution of 2-bromo-3,4-difluoro-1-nitrobenzene (12.0 g, 50.6 mmol) in DMSO (100 mL) was added MeONa (5.5 g, 101.2 mmol, 2.0 eq) in portions at room temperature. Then the reaction mixture was stirred for 3 h. After reaction completed by TLC analysis, the reaction mixture was poured in to water (300 mL) and extracted with EtOAc (200 mL×4). The combine organic layers were washed with water (500 mL×2) and brine (500 mL). The organic layer was dried over Na₂SO₄ and concentrated in vacuum to afford a crude product, which was purified by silica gel column chromatography (eluted with petroleum ether/EtOAc=50:1, v/v) to afford INT C5-1 (10.0 g, 79.7%) as a yellow solid. ¹HNMR (300 MHz, DMSO-d₆): δ 8.04 (d, J=9.0 Hz, 1H), 7.42 (t, J=9.0 Hz, 1H), 3.99 (s, 3H).

To a solution of INT C5-1 (10.0 g, 40.0 mmol) in EtOH/H₂O (5:1, 120 mL) was added NH₄Cl (8.5 g, 160 mmol) at room temperature. Then the reaction mixture was heated to 50° C. and Fe (12.2 g, 216.0 mmol) was added by portion wise. The reaction mixture was heated to reflux and stirred for 4 h. After reaction completed, the reaction mixture was filtered and the filtrate was concentrated. The residue was dissolved in EtOAc (200 mL) and washed with water (100 mL) and brine (100 mL). The organic layer was dried over Na₂SO₄ and concentrated in vacuum to afford a crude product, which was purified by silica gel column chromatography (eluted with petroleum ether/EtOAc=30:1) to afford INT C5-2 (3.6 g, 40.9%) as a yellow solid. ¹HNMR (300 MHz, CDCl₃): 8.05 (d, J=9.3 Hz, 1H), 7.42 (t, J=9.0 Hz, 1H), 3.99 (s, 3H).

A three-necked bottle treated with MsOH (20 mL) was heated to 125° C. and INT C5-2 (3.6 g, 16.4 mmol) was added portion wise into the reaction. Subsequently sodium 3-nitrobenzenesulfonate (2.2 g, 9.8 mmol) and FeSO₄·7H₂O (137 mg, 0.5 mmol) were added at this temperature. Followed by Glycerol (3.6 g, 39.3 mmol, 2.4 eq) was added dropwise to the reaction mixture and then the reaction solution was stirred for 12 h. Then the reaction mixture was cooled to room temperature and poured in ice-water (200 mL). The pH value of the mixture was adjusted to 14 with NaOH (1 M) and extracted with EtOAc (200 mL×2). The combined organic layers were washed with water (100 mL) and brine (100 mL). The organic layer was dried over Na₂SO₄ and concentrated in vacuum to afford a crude product, which was purified by silica gel chromatography (eluted with petroleum ether/EtOAc=5:1, v/v) to afford INT C5-3 (1.3 g, 31.0%) as a white solid.

Under nitrogen atmosphere a solution of INT C5-3 (400 mg, 1.56 mmol) in THF (5 mL) was cooled to −78° C., then n-BuLi (2.4 M, 1 mL, 2.3 mmol) was added dropwise to the reaction solution. Then the reaction mixture was stirred for 30 min at this temperature. After that 2-isopropoxy-4,4,5,5-tetramethyl-1,3-dioxolane (587 mg, 3.12 mmol) was added dropwise. Then the reaction mixture was allowed to warm to room temperature and stirred for 2 h. The reaction mixture was quenched with water (5 mL) and extracted with EtOAc (10 mL×2). The combine organic layer was washed with brine (10 mL), dried over Na₂SO₄ and concentrated in vacuum to afford a crude product, which was purified by silica gel column chromatography (eluted with petroleum ether/EtOAc=10:1, v/v) to afford INT C5-4 (280 mg, 81.2%) as a white solid. ¹HNMR (300 MHz, DMSO-d₆): δ 9.41 (d, J=1.8 Hz, 1H), 8.51 (dd, J=8.1, 1.5 Hz, 1H), 7.77-7.26 (m, 1H), 7.43 (d, J=9.0 Hz, 1H), 3.86 (s, 3H). MS (ESI, m/z): 222 [M+H]⁺.

Under nitrogen atmosphere a solution of INT C5-4 (100 mg, 0.45 mmol) in DCM (1 mL) was cooled to 0° C., then a solution of BBr₃ in DCM (1 M, 2.3 mL, 2.3 mmol) was added dropwise to the reaction solution. Then the reaction mixture was allowed to warm to room temperature and stirred for 2 h. The reaction mixture was quenched with water (2 mL), white solid was precipitated out. The mixture was filtered and the solid was dried to afford INT C5 (63 mg, 67.7%) as a white solid, which was used directly for next step without further purification. MS (ESI, m/z): 208 [M+H]⁺.

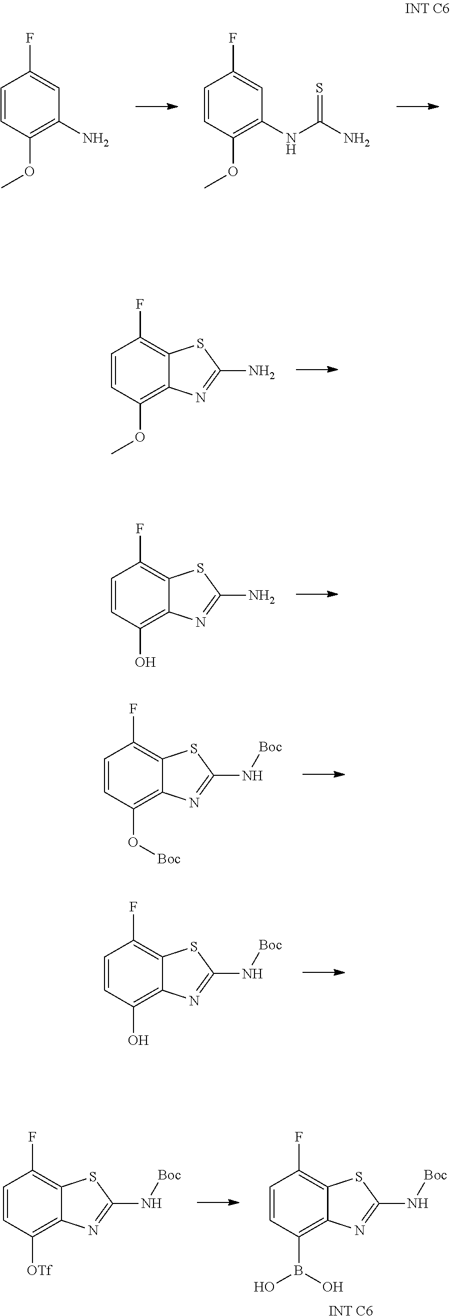

INT C6 was synthesized with 5-fluoro-2-methoxyaniline as starting material through using conventional preparation method.

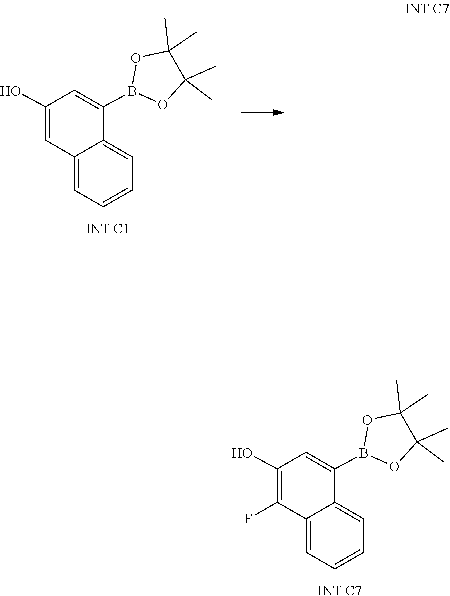

To a solution of INT C1 (420 mg, 1.55 mmol) in MeCN (12 mL) was added selectfluor fluorinating reagent (813 mg, 2.29 mmol) at 0° C. and stirred for 2 h. The reaction mixture was concentrated under reduced pressure. The residue was purified by Pre-TLC (Hex:EA=10:1, v/v) to afford INT C7 (177 mg, 0.62 mmol). MS: m/z 287 [M−1]⁺.

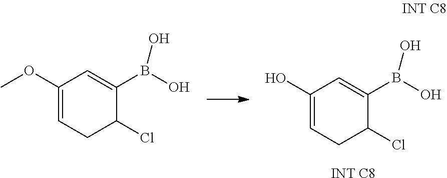

A solution of (2-chloro-5-methoxyphenyl)boronic acid (4.0 g, 21.4 mmol) in DCM (204 mL) at 0° C. was added BBr₃ (14.9 g, 59.5 mmol) slowly dropwise. The reaction mixture was warmed to 25° C. and stirred overnight. Upon completion, the reaction mixture was poured into ice-water, then 3 M NaOH (60 mL) was added, the mixture was stirred for 30 minutes. The layers were separated, the aqueous layer was adjusted to pH about 5 with concentrated HCl, extracted with EtOAc (150 Ml×3), dried over Na₂SO₄ and concentrated to afford INT C8 (3.2 g, 18.6 mmol) as a white solid.

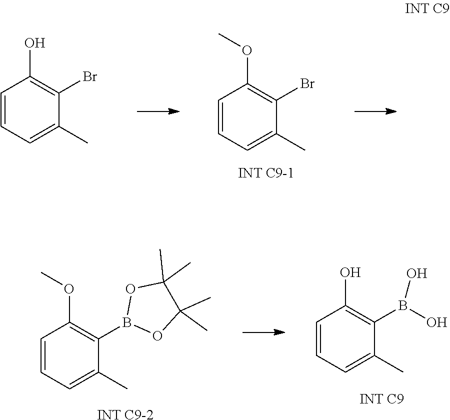

To a solution of 2-bromo-3-methylphenol (3.3 g, 17.6 mmol) and KOH (1.5 g, 26.5 mmol) in MeCN (36 mL) was added MeI (12.5 g, 88.2 mmol) dropwise at 0° C. under argon. Then the reaction mixture was stirred at 24° C. for 14 h. After that the reaction mixture was poured into water (50 mL) and extracted with EtOAc (100 mL×2). The organic layer was washed with water (50 mL), brine (50 mL) and concentrated in vacuum to afford a crude product which was purified by silica gel chromatography (eluted with petroleum ether/EtOAc=5:1, v/v) to afford INT C9-1 (3.0 g, 84.6%) as a white solid. ¹HNMR (300 MHz, DMSO-d₆): δ 7.24 (t, J=7.8 Hz, 1H), 6.93 (dd, J=7.2, 1.5 Hz, 2H), 3.83 (s, 3H), 2.35 (s, 3H).

To a solution of INT C9-1 (1.0 g, 5.0 mmol) in THF (25 mL) was added n-BuLi (2.4 M, 3.1 mL, 7.5 mmol) dropwise at −60° C. under argon. Then the reaction mixture was stirred at −60° C. for 1 h. Then 2-isopropoxy-4,4,5,5-tetramethyl-1,3,2-dioxaborolane (1.8 g, 9.9 mmol) was added dropwise at −60° C. under argon. The reaction mixture was stirred at room temperature for 1 h and quenched with water (50 mL), followed by extracted with EtOAc (50 mL×3). The combined organic layers were washed with water (50 mL), brine (50 mL) and concentrated in vacuum to afford a crude product which was purified by reversed C₁₈ (eluted with MeCN/H₂O=13:7, v/v) to afford INT C9-2 (800 mg, 64.8%) as a white solid. ¹HNMR (300 MHz, DMSO-d₆): δ 7.21 (t, J=8.1 Hz, 1H), 6.73 (d, J=7.8 Hz, 2H), 3.69 (s, 3H), 2.24 (s, 3H), 1.30 (s, 12H). MS: m/z 249 [M+1]⁺.

To a solution of INT C9-2 (500 mg, 2.0 mmol) in DCM (6 mL) was added BBr₃ in DCM (1.0 M, 6.0 mL, 6.0 mmol) dropwise at 0° C. under argon. The reaction mixture was stirred at room temperature for 1 h and quenched with ice water (10 mL) and extracted with EtOAc (30 mL×2). The combined organic layers were washed with water (10 mL×1), brine (10 mL×1) and concentrated in vacuum to afford INT C9 (800 mg, crude) as a yellow solid which was used to next step without further purification. MS: m/z 153 [M+1]⁺.

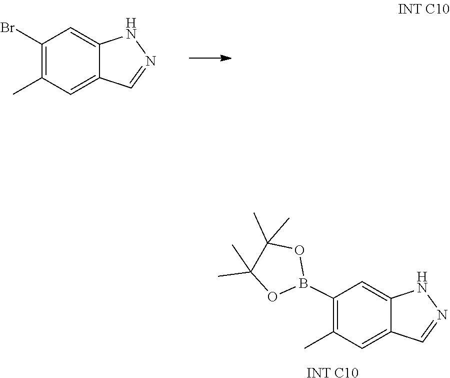 [SMILES: Cc1cc2cn[nH]c2cc1Br ; "INT C10" ; Cc1cc2cn[nH]c2cc1B1OC(C)(C)C(C)(C)O1]

6-Bromo-5-methyl-1H-indazole (200 mg, 0.95 mmol), B₂(Pin)₂ (361 mg, 1.4 mmol), KOAc (280 mg, 2.8 mmol) and Pd(dppf)Cl₂ (70 mg, 0.09 mmol) were placed in an oven-dried Schlenk-tube. The tube was evacuated and filled with argon for three times. Then 1,4-dioxane (5 mL) was added at room temperature. The mixture was stirred at 100° C. for 24 h. Upon completion, water (5 mL) was added to the reaction mixture, extracted with EtOAc (3×5 mL), and the combined organic layers were washed with saturated brine and dried over anhydrous sodium sulfate. The solution was concentrated to afford a crude residue. Purified by silica gel column chromatography (Petroleum ether/ethyl acetate=5/1) to afford INT C10 as a colorless liquid. MS: m/z 259 [M+1]⁺.

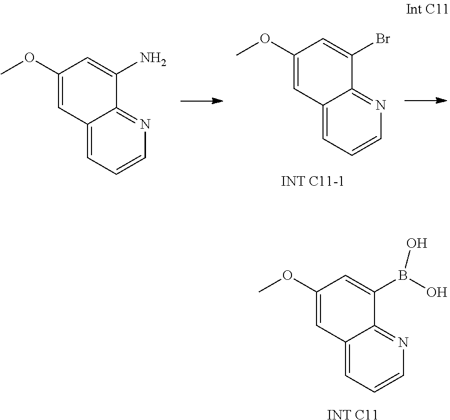

To a solution of 6-methoxyquinolin-8-amine (3.5 g, 20.1 mmol) in 40% HBr (20 mL), NaNO₂ (1.8 g, 26.1 mmol) and H₂O (14 ml) was added at 0° C. After stirring at 0° C. for 20 min, the resulting mixture was added dropwise to a 30° C. solution of CuBr (3.5 g, 24 mmol) and 40% HBr (32.3 mL). After 30 min, the reaction mixture was neutralized with 105 ml of ice-cold 5 M NaOH solution, EtOAc (210 ml) was added and the resulting mixture was stirred for about 10 min, then filtered. The filtrate was extracted EtOAc (54 mL×2), and the combined organic layers were washed with H₂O (54 mL), brine (54 mL), dried over Na₂SO₄, filtered, and concentrated to afford a residue. Purified by silica gel column chromatography (Petroleum ether/ethyl acetate=30/1, v/v) to afford INT C11-1 (560 mg, 2.4 mmol) as a yellow solid. ¹H NMR (300 MHz, CDCl₃): δ 8.89 (dd, J=4.2, 1.6 Hz, 1H), 8.05 (dd, J=8.3, 1.6 Hz, 1H), 7.76 (d, J=2.7 Hz, 1H), 7.40 (dd, J=8.3, 4.2 Hz, 1H), 7.06 (d, J=2.7 Hz, 1H), 3.92 (s, 3H). MS: m/z 238 [M+1]⁺.

INT C11-1 (370 mg, 1.7 mmol), B₂(Pin)₂ (660 mg, 2.6 mmol), KOAc (500 mg, 5.1 mmol), Pd(dppf)Cl₂ (124 mg, 0.17 mmol) were placed in an oven-dried Schlenk-tube. The tube was evacuated and filled with argon for three times. Then 1,4-dioxane (6 mL) was added at room temperature. The mixture was stirred at 100° C. for 2 hours. Upon completion, water (about 20 mL) was added, the mixture is extracted with EtOAc (20 mL×3), HPLC showed that INT C11 in the water layer has higher purity than the EtOAc layer, the water layer was extracted by CHCl₃/iPr-OH (3/1, 20 mL×3), dried over Na₂SO₄, filtered, and concentrated to afford INT C11 (170 mg, 0.83 mmol, 49% yield) as a brown solid. MS: m/z 204 [M+1]⁺.

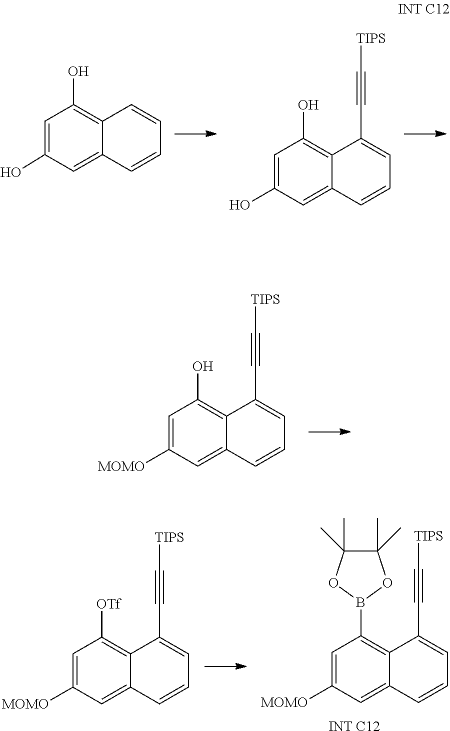

INT C12 was synthesized with naphthalene-1,3-diol as starting material through using conventional preparation method.

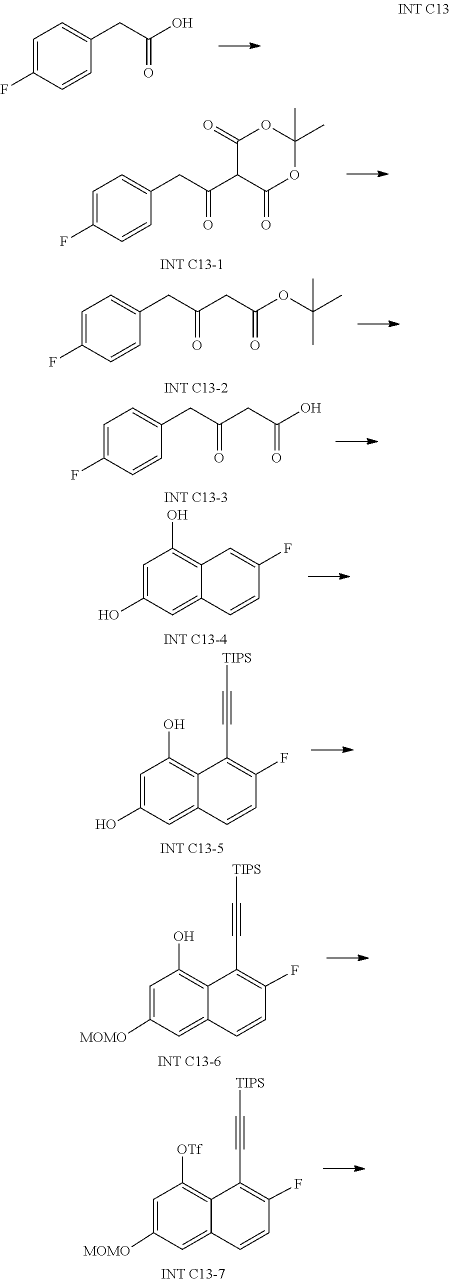

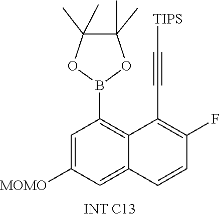

INT C13 was synthesized with 2-(4-fluorophenyl) acetic acid as starting material through using conventional preparation method.

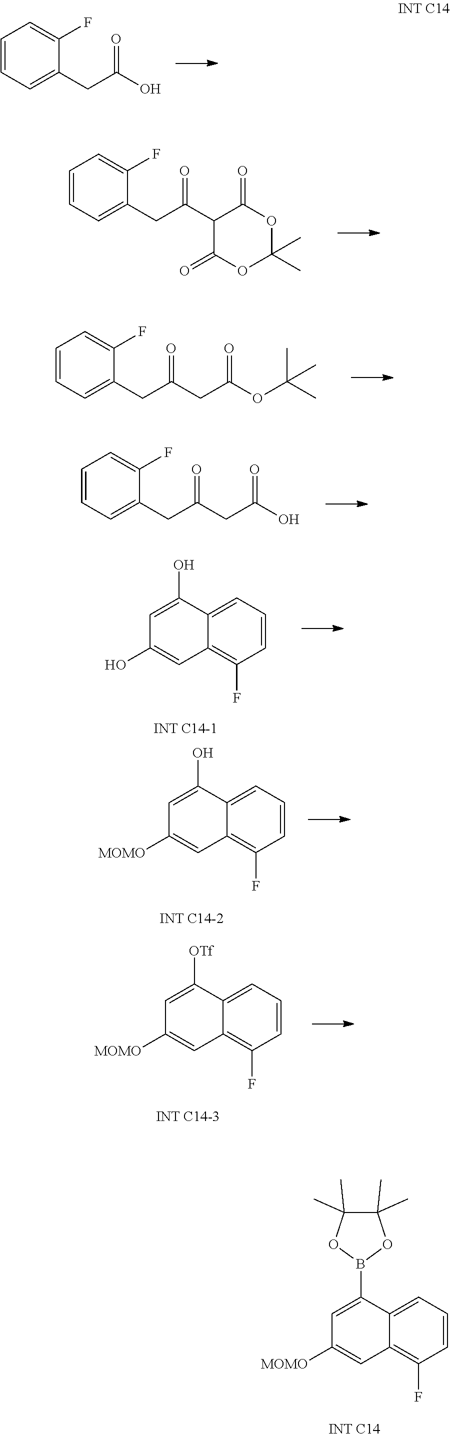

Following an analogous procedure described in procedure of INT C13-4, INT C14-1 was obtained with 2-(2-fluorophenyl)acetic acid as start material.

MOMCl (4.0 g, 49.2 mmol, 1.2 eq) was added dropwise in portions (first time: 2.0 g; second time: 1.0 g; third time: 1.0 g) to a mixture of INT C14-1 (7.3 g, 41.0 mmol, 1.0 eq) and DIEA (15.9 g, 123.0 mmol, 3.0 eq) in DCM (73 mL) at 0° C. The reaction mixture was stirred at 0° C. for 8 hrs, then poured into ice water (100 mL) after the reaction was completed, and the aqueous layer was extracted with DCM (100 mL×3). The organic layers were combined, dried over Na₂SO₄, filtered, and then concentrated to afford a crude containing INT C14-2 (2.9 g) as brown oil which was used directly for next step without any further purification. LCMS m/z=223.1 [M+H]⁺.

Tf₂O (3.6 g, 12.9 mmol, 1.5 eq) was added dropwise to a solution of INT C14-2 (1.9 g, 8.6 mmol, 1.0 eq) and DIEA (3.3 g, 25.8 mmol, 3.0 eq) in DCM (28.5 mL) at −40° C. The reaction mixture was stirred at −40° C. for 30 mins, quenched with water (50 mL), and the aqueous layer was extracted with DCM (50 mL×3). The organic layers were combined, dried over Na₂SO₄, filtered and then concentrated to afford a crude containing INT C14-3 (3.3 g) as yellow oil which was used directly for next step without any further purification.

A mixture of the crude containing INT C14-3 (2.1 g, 5.9 mmol, 1.0 eq), Bis(pinacolato)diborane (3.0 g, 11.8 mmol, 1.5 eq), Pd(dppf)Cl₂ (431.7 mg, 0.59 mmol, 0.1 eq), KOAc (1.7 g, 17.7 mmol, 3.0 eq) and toluene (21 mL) was stirred at 130° C. for 4 hrs under N₂ atmosphere, filtered after cooling to room temperatures and then concentrated under reduced pressure to obtain a residue. The residue was purified by silica gel chromatography (Petroleum ether/EtOAc=60:1) to afford INT C14 (400 mg, 1.2 mmol, 13.8%) as a yellow solid.

¹H NMR (300 MHz, CDCl₃): δ 8.44 (d, J=8.5 Hz, 1H), 7.82 (d, J=2.7 Hz, 1H), 7.71 (d, J=2.5 Hz, 1H), 7.45-7.28 (m, 1H), 7.16-7.06 (m, 1H), 5.32 (s, 2H), 3.52 (s, 3H), 1.42 (s, 12H). LCMS: 223.1 ([M+H]⁺).

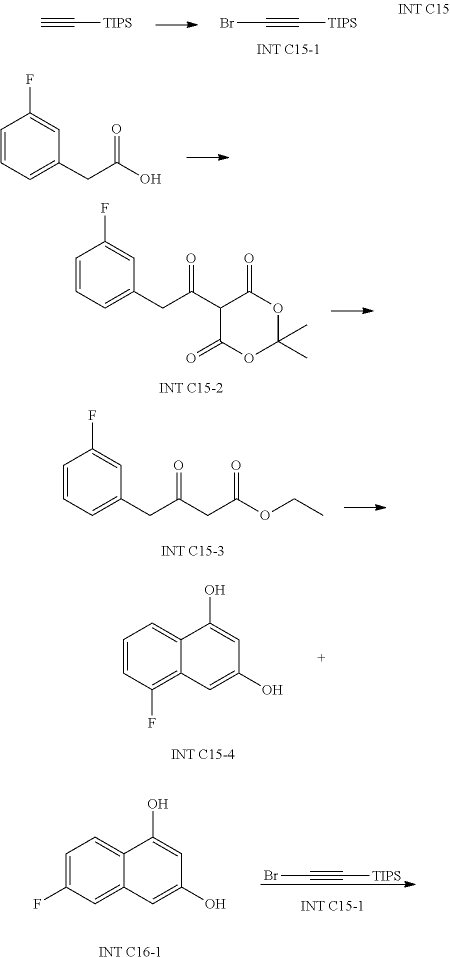

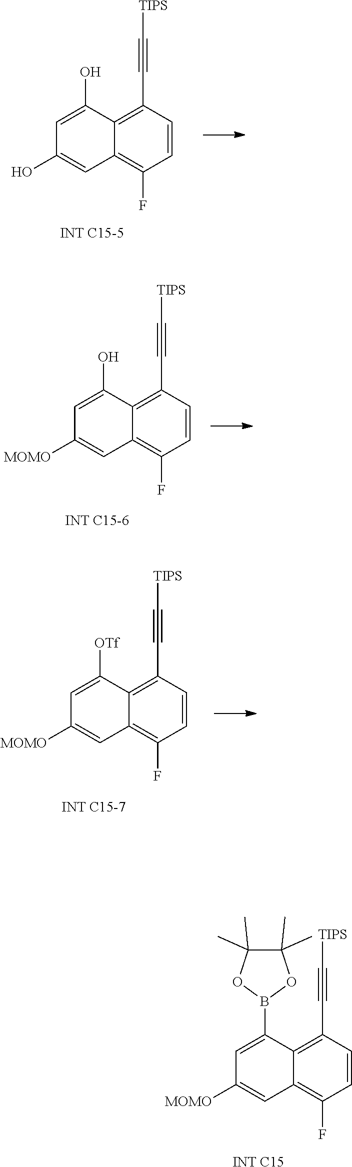

INT C15-1 was synthesized with ethynyltriisopropylsilane as starting material through using conventional preparation method.

Maintaining the temperature below 45° C., DIEA (90.1 g, 697.4 mmol, 2.2 eq) was added to a solution of 2-(3-fluorophenyl)acetic acid (50.0 g, 324.4 mmol, 1.0 eq), 2,2-Dimethyl-1,3-dioxane-4,6-dione (51.4.0 g, 356.8 mmol, 1.1 eq) and DMAP (3.4 g, 27.6 mmol, 0.09 eq) in MeCN (150 mL), and then pivaloyl chloride (43.0 g, 356.8 mmol, 1.1 eq) was slowly added over 3 hrs. The reaction mixture was stirred at 45° C. for 3 hrs, cooled to 0° C., and then aq. HCl (1 M, 500 mL) was slowly added. The resulting solution was stirred at 0° C. for 2 hrs and a solid was precipitated. The solid was collected by filtration and dried to afford a crude product which was washed with diethyl ether (300 mL) to obtain INT C15-2 (89.0 g, 97.9%) as a white solid. ¹H NMR (300 MHz, CDCl₃): δ 15.34 (s, 1H), 7.30-7.15 (m, 4H), 4.41 (s, 2H), 1.72 (s, 6H). LCMS: 279.1 ([M−H]⁻).

A solution of INT C15-2 (25.0 g, 89.2 mmol, 1.0 eq) in EtOH (75 mL) was stirred at 90° C. for 2 hrs, and then concentrated to obtain a crude containing INT C15-3 (20.0 g, 99.0%) as yellow oil. LCMS: 225.1 ([M+H]⁺).

INT C15-3 (20.0 g, 89.2 mmol, 1.0 eq) was added by portion wise to concentrated H₂SO₄ (65.5 g, 668.6 mmol, 7.5 eq) at 0° C. The reaction mixture was stirred at room temperature for 24 hrs, cooled to 0° C. and poured slowly into ice-water (300 mL). The resulting mixture was filtered, and the filter cake was dispersed in petroleum ether (100 mL). The solid was collected by filtration and dried to afford INT C15-4 (5.0 g) and INT C16-1 (7.7 g, 48.5%)

INT C16-1 ¹H NMR (400 MHz, DMSO-d₆): δ 10.22 (s, 1H), 9.63 (s, 1H), 7.97 (t, J=2.8 Hz, 1H), 7.32-7.29 (m, 1H), 7.03-6.98 (m, 1H), 6.57 (d, J=2.0 Hz, 1H), 6.45 (d, J=2.0 Hz, 1H). LCMS: 177.1 ([M−H]⁻).

A mixture of INT C15-4 (5.0 g, 28.1 mmol, 1.0 eq), INT C15-1 (7.5 g, 28.7 mmol, 1.03 eq), Dichloro(p-cymene) ruthenium(II) dimer (1.7 g, 2.8 mmol, 0.1 eq), AcOK (5.5 g, 56 mmol, 2.0 eq) and dioxane (35 mL) was purged with N₂ for three times, heated to 110° C. for 1.5 hrs, cooled to room temperature, and then filtered. The filtrate was concentrated to obtain a residue which was purified by silica gel chromatography (Petroleum ether/EtOAc=30:1 to 10:1) to afford INT C15-5 (10.1 g, 28.2 mmol, 98%) as black oil. ¹H NMR (300 MHz, DMSO-d₆): δ 10.26 (s, 1H), 9.92 (s, 1H), 7.29 (dd, J=8.0, 5.7 Hz, 1H), 7.12 (dd, J=10.7, 8.0 Hz, 1H), 6.69 (d, J=1.7 Hz, 1H), 6.60 (d, J=2.3 Hz, 1H), 1.12 (s, 21H). LCMS: 357.2 ([M−H]⁻).

MOMCl (1.3 g, 16.4 mmol, 1.3 eq) was added dropwise to a mixture of INT C15-5 (4.5 g, 12.6 mmol, 1.0 eq), DIEA (4.9 g, 37.8 mmol, 3.0 eq) and DCM (120 mL) at 0° C. The reaction mixture was stirred at 0° C. for 3 hrs, poured into ice water (100 mL), and the aqueous layer was extracted with DCM (100 mL×2). The organic layers were combined, washed with brine (100 mL), dried over Na₂SO₄, filtered and then concentrated to afford a residue which was purified by silica gel chromatography (Petroleum ether/EtOAc=80:1) to obtain INT C15-6 (3.1 g, 7.7 mmol, 29.6%) as yellow oil. ¹H NMR (300 MHz, CDCl₃): δ 9.31 (s, 1H), 7.42 (dd, J=8.0, 5.5 Hz, 1H), 7.17 (d, J=2.4 Hz, 1H), 7.02 (dd, J=10.1, 8.0 Hz, 1H), 6.81 (d, J=2.4 Hz, 1H), 5.28 (s, 2H), 3.51 (s, 3H), 1.18 (s, 21H). LCMS: 401.2 ([M−H]⁻).

Tf₂O (2.1 g, 7.4 mmol, 1.5 eq) was added dropwise to a solution of INT C15-6 (2.0 g, 4.9 mmol, 1.0 eq) and DIEA (1.9 g, 14.9 mmol, 3.0 eq) in DCM (30 mL) at −40° C. The reaction mixture was stirred at −40° C. for 30 mins, quenched with water (80 mL), and the aqueous layer was extracted with DCM (50 mL×2). The organic layers were combined, washed with brine, dried over Na₂SO₄, filtered and then concentrated to obtain a residue which was purified by silica gel chromatography (Petroleum ether/EtOAc=50:1 to 10:1) to afford INT C15-7 (3.8 g, 7.1 mmol, 93.5%) as yellow oil. ¹H NMR (300 MHz, CDCl₃): δ 7.74-7.61 (m, 2H), 7.36 (d, J=2.3 Hz, 1H), 7.15 (dd, J=9.8, 8.2 Hz, 1H), 5.31 (s, 2H), 3.52 (s, 3H), 1.21-1.12 (m, 21H).

A mixture of INT C15-7 (3.0 g, 5.6 mmol, 1.0 eq), Bis(pinacolato)diborane (2.1 g, 8.4 mmol, 1.5 eq), Pd(dppf)Cl₂—CH₂Cl₂ (228 mg, 0.28 mmol, 0.05 eq), dppf (155.2 mg, 0.28 mmol, 0.05 eq), KOAc (1.7 g, 16.9 mmol, 3.02 eq) and dioxane (22 mL) was stirred overnight at 110° C. under N₂ atmosphere, cooled to room temperature, filtered and then concentrated under reduced pressure to obtain a residue. The residue was purified by silica gel chromatography (Petroleum ether/EtOAc=80:1) to afford INT C15 (670 mg, 1.3 mmol, 23.3%) as a yellow solid. ¹H NMR (300 MHz, DMSO-d₆): δ 7.63-7.59 (m, 2H), 7.50 (d, J=2.4 Hz, 1H), 7.08-7.02 (m, 1H), 5.31 (s, 2H), 3.50 (s, 3H), 1.40 (s, 12H), 1.14 (s, 21H). LCMS 513.0 ([M+H]⁺).

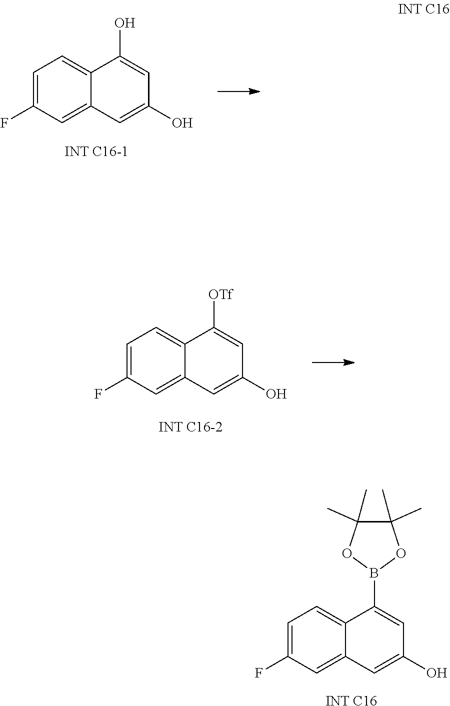 [SMILES: "INT C16-1" ; "INT C16-2" ; Oc1cc(O)c2ccc(F)cc2c1 ; "INT C16" ; O=S(=O)(Oc1cc(O)cc2cc(F)ccc12)C(F)(F)F ; CC1(C)OB(c2cc(O)cc3cc(F)ccc23)OC1(C)C]

NaH (1.08 g, 26.9 mmol, 1.2 eq) was added by portion wise to a solution of INT C16-1 (4.0 g, 22.5 mmol, 1.0 eq) in DCM (60 mL). The reaction mixture was stirred at 0° C. for 10 mins, and then Tf₂O (3.8 g, 13.47 mmol, 0.6 eq) was added dropwise. The reaction mixture was stirred at room temperature for 30 mins, quenched with water (10 mL), and the aqueous layer was extracted with DCM (10 mL×2). The organic layers were combined, washed with brine and dried over Na₂SO₄, filtered and then concentrated to obtain a crude product which was purified by silica gel chromatography (eluted with petroleum ether/EtOAc=80:1 to 10:1) to afford INT C16-2 (1.6 g, 23.0%) as yellow oil. ¹H NMR (300 MHz, DMSO-d₆): δ 11.30 (s, 1H), 8.27 (dd, J=9.3 Hz, 5.7 Hz, 1H), 7.83 (dd, J=10.2 Hz, 2.4 Hz, 1H), 7.54-7.47 (m, 2H), 6.84 (d, J=2.4 Hz, 1H). LCMS: 309.0 ([M−H]⁻).

Pd(dppf)Cl₂ (377 mg, 0.01 mmol, 0.01 eq) and KOAc (1.8 g, 18.1 mmol, 3.5 eq) were added to a solution of INT C16-2 (1.6 g, 5.2 mmol, 1.0 eq), Bis(pinacolato)diborane (2.6 g, 10.3 mmol, 2.0 eq) in toluene (13 mL). The reaction mixture was stirred overnight at 110° C. under N₂ atmosphere, cooled to room temperature, filtered and then concentrated under reduced pressure to obtain a residue which was purified by pre-HPLC to afford INT C16 (700.0 mg, 47.1%) as a white solid. ¹H NMR (300 MHz, DMSO-d₆): δ 9.85 (s, 1H), 8.57-8.52 (m, 1H), 7.56-7.48 (m, 2H), 7.25-7.20 (m, 2H), 11.38 (s, 12H). LCMS: 287.0 ([M−H]⁻).

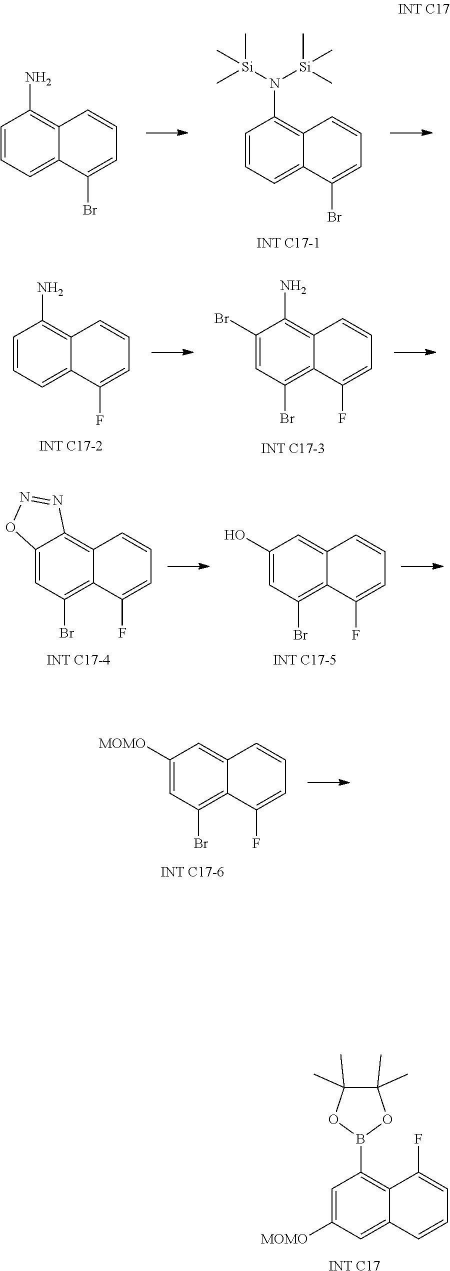

LiHMDS (90 mL, 1.0 M, 2.2 eq) was added to a solution of 5-bromonaphthalen-1-amine (9.0 g, 40.5 mmol, 1.0 eq) in THF (130 mL) under argon at −78° C., and the reaction mixture turned into a dark red solution immediately. After warming the reaction mixture to room temperature and stirring for 10 mins, TMSCl (9.2 g, 85.1 mmol, 2.1 eq) was added dropwise at −78° C. The resulting mixture was slowly warmed to 20° C., stirred for 1 h, and then concentrated to obtain a residue which was washed with hexanes and filtered. The filtrate was concentrated to afford a crude containing INT C17-1 (17.0 g, crude) as a brown liquid which was directly used for the next step without further purification. ¹H NMR (300 MHz, CDCl₃): δ 8.19-8.16 (m, 1H), 8.03-7.99 (m, 1H), 7.78-7.73 (m, 1H), 7.48-7.43 (m, 1H), 7.31-7.28 (m, 1H), 7.13-7.11 (m, 1H), 0.06-0.03 (m, 18H).

n-BuLi (2.4 M, 28.8 mL, 69.0 mmol, 1.5 eq) was added dropwise to a solution of the crude containing INT C17-1 (17.0 g, 46.4 mmol, 1.0 eq) in THF (200 mL) at −78° C. under argon. The reaction mixture was stirred for 30 mins and then NFSI (23.3 g, 74.0 mmol, 1.6 eq) was added. The resulting mixture was slowly warmed to 20° C. for 1 h, quenched with water (200 mL) and extracted with EtOAc (200 mL×2). The organic layers were combined, dried over Na₂SO₄ and concentrated under reduced pressure to afford a residue. The residue was dispersed in MeOH (100 mL) and HCl (1.0 M, 50 mL) for 3 mins to obtain a mixture. The mixture was concentrated, basified with NaHCO₃ and then extracted with EtOAc (200 mL×2). The organic layers were combined, washed with brine (200 mL), dried over Na₂SO₄ and concentrated under reduced pressure to obtain a residue which was purified by pre-HPLC (eluting with MeCN in water (0-95% with 0.1 TFA)) to afford INT C17-2 (6.5 g, crude) as a brown solid. LCMS: 162.0 ([M+H]⁺).

A solution of Br₂ (12.8 g, 80.6 mmol, 2.0 eq) in AcOH (26 mL) was added to a mixture of C17-2 (6.5 g, 40.3 mmol, 1.0 eq) in AcOH (45 mL) at 0° C. The reaction mixture was heated to 70° C. and stirred for 1 h, and then filtered. The filter cake was washed with AcOH (100 mL), and 15% aqueous of NaOH (50 mL) was added. The resulting mixture was stirred for 20 mins and filtered to obtain a solid. The solid was washed with water (100 mL) and dried under vacuum to afford INT C17-3 (5.0 g, yield 38.8% for three steps) as a black solid. ¹H NMR (400 MHz, DMSO-d₆): δ 8.13 (d, J=8.4 Hz, 1H), 7.80-7.79 (m, 1H), 7.57-7.48 (m, 1H), 7.43-7.48 (m, 1H). LCMS: 319.8 ([M+H]⁺).

Propionic acid (12.5 g, 168.0 mmol, 10.7 eq) was added to a solution of INT C17-3 (5.0 g, 15.7 mmol, 1.0 eq) in AcOH (100 mL) at 0° C., and then NaNO₂(1.6 g, 23.6 mmol, 1.5 eq) was added. The reaction mixture was stirred at 0° C. for 1 h, warmed up to 20° C. for 1 h, and then filtered. The filter cake was washed with water (100 mL) and dried under vacuum to afford INT C17-4 (3.0 g, yield 71.4%) as a yellow solid. LCMS: 266.9, 268.9 ([M+H]⁺).

NaBH₄ (850 mg, 22.5 mmol, 2.0 eq) was added to a solution of INT C17-4 (3.0 g, 11.2 mmol, 1.0 eq) in EtOH (40 mL) and THF (20 mL) at 0° C. The reaction mixture was stirred at 0° C. for 1 h, quenched with KHSO₄ (10%, 50 mL) and concentrated under reduced pressure, poured into water (30 mL) and extracted with EtOAc (30 mL×2). The organic layers were combined, dried over Na₂SO₄ and concentrated under reduced pressure to afford a crude product which was purified by silica gel chromatography (petroleum ether/EtOAc=50:1) to afford INT C17-5 (1.0 g, yield 37.0%) as a black solid. LCMS: 239.0, 241.0 ([M−H]⁻).

MOMCl (594 mg, 7.4 mmol, 1.8 eq) was added dropwise to a solution of INT C17-5 (1.0 g, 4.1 mmol, 1.0 eq) and DIEA (1.3 g, 10.3 mmol, 2.5 eq) in DCM (25 mL) at 0° C. The reaction mixture was stirred at 0° C. for 1 h, heated to room temperature and poured into water (20 mL) and extracted with DCM (20 mL×2). The organic layers were combined, dried over Na₂SO₄ and concentrated under reduced pressure to afford a crude product which was purified by silica gel chromatography (eluted with petroleum ether/EtOAc=50:1) to afford INT C17-6 (1.0 g, yield 83.3%) as a yellow solid. ¹H NMR (300 MHz, CDCl₃): δ 7.57 (d, J=2.4 Hz, 1H), 7.54-7.51 (m, 1H), 7.40-7.33 (m, 2H), 7.11-7.04 (m, 1H), 5.36 (s, 2H), 3.51 (s, 3H).

A mixture of INT C17-6 (1.0 g, 3.5 mmol, 1.0 eq), Bis(pinacolato)diborane (2.2 g, 8.8 mmol, 2.5 eq), AcOK (1.0 g, 10.5 mmol, 3.0 eq), Pd(dppf)Cl₂ (256.0 mg, 0.35 mmol, 0.1 eq), and 1,4-dioxane (20.0 mL) was purged with argon for 10 mins, stirred for 1 h at 110° C. under argon atmosphere, then cooled to room temperature, poured into water (30 mL) and extracted with EtOAc (30 mL×2). The organic layers were combined, dried over Na₂SO₄ and concentrated under reduced pressure to afford crude product which was purified by silica gel chromatography (eluted with petroleum ether/EtOAc=50:1) to obtain INT C17 (1.1 g, crude) as a yellow solid. ¹H NMR (300 MHz, CDCl₃): δ 7.51-7.48 (m, 1H), 7.41-7.39 (m, 1H), 7.36-7.29 (m, 2H), 7.04-6.97 (m, 1H), 4.29 (s, 2H), 3.50 (s, 3H), 1.43 (s, 12H).

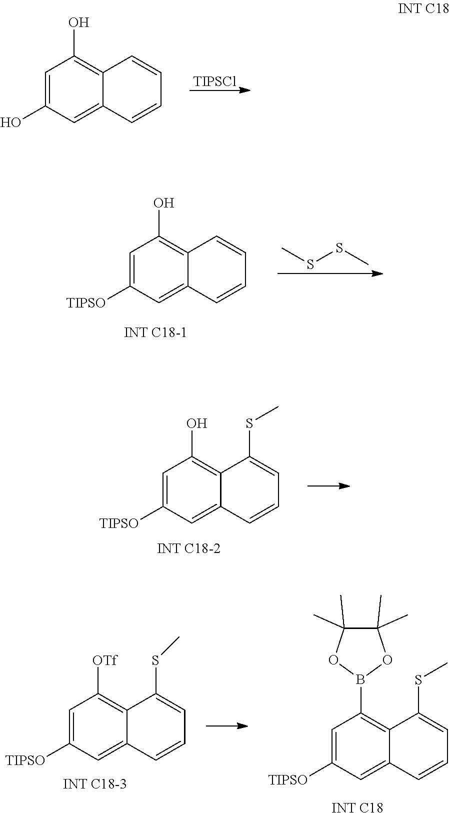

A mixture of naphthalene-1,3-diol (5.06 g, 31.59 mmol), 1H-imidazole (3.18 g, 46.71 mmol), chlorotriisopropylsilane (6.26 g, 32.47 mmol) and DMF (32 mL) was stirred overnight at R.T, and then water (50 mL) and MTBE (50 mL) were added. The organic layer was washed with water (30 mL), brine (30 mL) successively, dried over Na₂SO₄ and concentrated under reduced pressure to obtain a residue which was purified with silica gel chromatography (eluting with EA:Hex=0%˜10%) to afford INT C18-1 (3.40 g, 10.74 mmol) as light yellow oil. MS m/z: 317 [M+H]⁺.

N,N,N′,N′-Tetramethylethylenediamine (1.28 g, 11.02 mmol) was added dropwise to a solution of n-butyllithium (2.5 M, 15 mL, 37.50 mmol) in Hex (5 mL) and the mixture was stirred at RT for 30 min. The reaction mixture was cooled to −20° C. and a solution of INT C18-1 (3.40 g, 10.74 mmol) in THF (5 mL) was added dropwise over 10 min. The reaction mixture was stirred for 24 hrs at R.T, cooled to −10° C. and 1,2-dimethyldisulfane (3.05 g, 32.38 mmol) was added dropwise. The resulting mixture was warmed to R.T over 10 mins, diluted with MTBE (50 mL), quenched with aq. HCl (1 M) and the pH of the resulting mixture was adjusted to 4. The organic layer was separated and concentrated under reduced pressure to obtain a residue which was purified with silica gel chromatography (eluting with EA:Hex=1%) to afford INT C18-2 (1.70 g, 4.69 mmol) as yellow oil. MS m/z: 363[M+H]⁺.

Trifluoromethanesulfonic anhydride (1.55 g, 5.48 mmol) was added dropwise to a solution of INT C18-2 (1.33 g, 3.67 mmol) and DIEA (1.21 g, 9.36 mmol) in DCM (30 mL) at −78° C. The reaction mixture was stirred at R.T for 2 hrs, then aq. NaHCO₃ (0.5 M, 50 mL) and hexane (50 mL) were added. The organic layer was washed with water (50 mL) and brine (50 mL) successively, dried over Na₂SO₄, concentrated under reduced pressure to obtain a residue which was purified with silica gel chromatography (eluting with EA:Hex=1:400, v/v) to afford INT C18-3 (1.32 g, 2.67 mmol) as yellow oil. MS m/z: 493[M−H]⁻.

A mixture of INT C18-3 (1.32 g, 2.67 mmol), 4,4,4′,4′,5,5,5′,5′-octamethyl-2,2′-bi(1,3,2-dioxaborolane) (2.06 g, 8.10 mmol), KOAc (789 mg, 8.04 mmol), Pd(dppf)Cl₂·CH₂Cl₂ (220 mg, 0.30 mmol) and 1,4-dioxane (13 mL) was purged with nitrogen for three times and stirred at 80° C. for 4 hrs. The reaction mixture was cooled to R.T and then NaHCO₃ (0.5 M, 50 mL) and MTBE (50 mL) were added. The organic layer was washed with water (50 mL) and brine (50 mL) successively, dried over Na₂SO₄, concentrated under reduced pressure to obtain a residue which was purified with silica gel chromatography (eluting with EA:Hex=0.5%) to afford INT C18 (752 mg, 1.59 mmol) as yellow oil. MS m/z: 473[M+H]⁺.

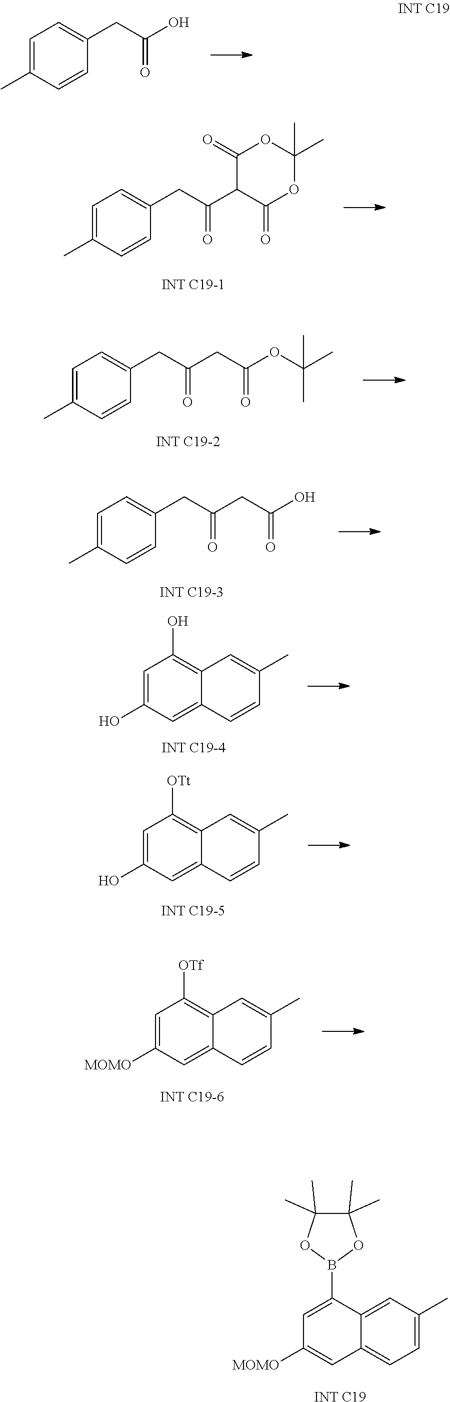

Maintaining temperature below 40° C., DIEA (46.3 g, 357.9 mmol, 2.15 eq.) and pivaloyl chloride (22.1 g, 183.1 mmol, 1.1 eq.) were slowly added successively to a mixture of 2-(p-tolyl)acetic acid (25.0 g, 166.5 mmol, 1.0 eq.), 2,2-dimethyl-1, 3-dioxane-4,6-dione (26.4 g, 183.1 mmol, 1.1 eq.), DMAP (1.7 g, 14.1 mmol, 0.085 eq.) and MeCN (75 mL). The reaction mixture was stirred at 45° C. for 3 hrs, cooled to 0° C., then 1 M HCl (250 mL) was slowly added. After stirring for 2 hrs at 0° C., the solid was collected by filtering, washed with diethyl ether (50 mL), and dried to afford INT C19-1 (41.6 g, 90.5%) as a yellow solid. ¹H NMR (300 MHz, DMSO-d₆): δ 7.22-7.12 (m, 4H), 4.32 (s, 2H), 2.28 (s, 3H), 1.69 (s, 6H). LCMS: 277 ([M+H]⁺).

A mixture of INT C19-1 (41.6 g, 150.6 mmol, 1.0 eq.) and t-BuOH (120 mL) was stirred at 90° C. for 2 hrs, and then concentrated to afford a crude containing the INT C19-2 (37.0 g, 98.9%) as yellow oil which was used into next step without any further purification. ¹H NMR (300 MHz, DMSO-d₆): δ 7.15-7.05 (m, 4H), 3.79 (s, 2H), 3.51 (s, 2H), 2.32 (s, 3H), 1.43 (s, 9H). LCMS: 271.1 ([M+Na]⁺).

A solution of INT C19-2 (37.0 g, 192.5 mmol, 1.0 eq.) and TFA (149.5 g, 1.31 mol, 6.81 eq.) in DCM (80 mL) was stirred at 20° C. for 1 h, and concentrated to obtain a crude product. The crude product was slurried with petroleum ether (40 mL) at room temperature for 1 h, and then filtered to afford INT C19-3 (23.0 g, 80.3%) as a white solid. ¹H NMR (300 MHz, DMSO-d₆): δ 7.14-7.06 (m, 4H), 3.81 (s, 2H), 3.48 (s, 2H), 2.28 (s, 3H). LCMS: 193.1 ([M+H]⁺).

A mixture of INT C19-3 (23.0 g, 119.7 mmol, 1.0 eq.) and CF₃SO₃H (449.0 g, 2.99 mol, 25.0 eq.) was stirred overnight at 25° C., cooled to 0° C. and added slowly into ice/water (750 mL). The solid was collected by filtration, dried, slurried with petroleum ether (100 mL), and then filtered to afford INT C19-4 (19.0 g, 91.3%) as a yellow solid. ¹H NMR (400 MHz, DMSO-d₆): δ 9.93 (brs, 1H), 9.30 (brs, 1H), 7.73 (d, J 0.8 Hz, 1H), 7.46 (d, J=8.4 Hz, 1H), 7.18-7.16 (m, 1H), 6.53 (d, J=2.0 Hz, 1H), 6.46 (d, J=2.0 Hz, 1H), 2.39 (s, 1H). LCMS: 175.1 ([M+H]⁺).

DIEA (3.6 g, 27.6 mmol, 1.2 eq.) and Tf₂O (6.5 g, 23.0 mmol, 1.0 eq.) were added to a solution of INT C19-4 (4.0 g, 23.0 mmol, 1.0 eq.) in DCM (80 mL). The reaction mixture was stirred overnight at R.T, then quenched with water (40 mL) after the reaction was completed, and then the aqueous layer was extracted with DCM (40 mL×2). The organic layers were combined, washed with brine (40 mL×1), dried over Na₂SO₄, filtered and concentrated to afford a crude product which was purified with silica gel chromatography (Petroleum ether/EtOAc=100:1) to afford INT C19-5 (2.0 g, 28.6%) as yellow oil. ¹H NMR (300 MHz, DMSO-d₆): δ 10.99 (s, 1H), 7.95 (d, J=0.6 Hz, 1H), 7.88 (d, J=8.4 Hz, 1H), 7.48-7.45 (m, 2H), 6.81 (d, J=2.4 Hz, 1H), 2.51 (s, 3H). LCMS: 305.0 ([M−H]⁻).

MOMCl (683.3 mg, 8.5 mmol, 1.3 eq.) was added dropwise at 0° C. to a mixture of INT C19-5 (2.0 g, 6.5 mmol, 1.0 eq.), DIEA (2.5 g, 18.6 mmol, 3.0 eq.) and DCM (20 mL). The reaction mixture was stirred at R.T for 4 hrs, poured into ice/water (20 mL), and extracted with DCM (20 mL×2). The organic layers were combined, washed with brine (20 mL), dried over Na₂SO₄, filtered and concentrated to afford a crude product containing INT C19-6 (2.0 g, 87.3%) as yellow oil. ³H NMR (300 MHz, DMSO-d₆): δ 8.00 (d, J=0.6 Hz, 1H), 7.95 (d, J=8.4 Hz, 1H), 7.68 (d, J=2.1 Hz, 1H), 7.53-7.50 (m, 1H), 7.13 (d, J=2.4 Hz, 1H), 5.48 (s, 2H), 3.47 (s, 3H), 2.49 (s, 3H).

A mixture of INT C19-6 (3.8 g, 10.9 mmol, 1.0 eq.), Bis(pinacolato)diborane (5.5 g, 21.7 mmol, 2.0 eq.), Pd(dppf)Cl₂ (794 mg, 1.1 mmol, 0.1 eq.) and KOAc (3.7 g, 38.0 mmol, 3.5 eq.) and toluene (50 mL) was stirred overnight at 110° C. under N₂ atmosphere, cooled to room temperature after the reaction was completed, filtered and then concentrated under reduced pressure to afford a residue which was purified with silica gel chromatography (eluting with Petroleum ether/EtOAc=200:1 to 50:1) to afford INT C19 (1.37 g, 38.3%) as a yellow solid. ¹H NMR (300 MHz, CDCl₃): δ 8.04 (d, J=0.6 Hz, 1H), 7.99 (s, 1H), 7.77-7.74 (m, 1H), 7.34-7.33 (m, 2H), 5.44 (s, 2H), 3.57 (s, 3H), 2.40 (s, 3H), 1.38 (s, 12H). LCMS: 329.2 ([M+H]⁺).

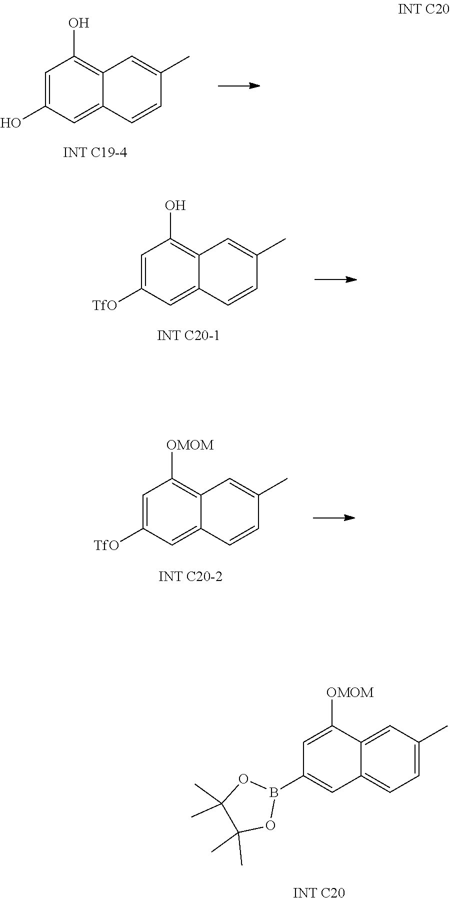

INT C20-1 was another product obtained during the preparing of INT C19-5 from INT C19-4. ¹H NMR (300 MHz, DMSO-d6): δ 10.31 (s, 1H), 7.80 (d, J=8.4 Hz, 1H), 7.56 (s, 1H), 7.43-7.40 (m, 1H), 7.27 (d, J=1.8 Hz, 1H), 7.19 (d, J=2.4 Hz, 1H), 2.47 (s, 3H). LCMS: 305.0 ([M−H]⁻).

MOMCl (208.4 mg, 2.6 mmol, 1.3 eq.) was added dropwise to a mixture of INT C20-1 (610.0 mg, 2.0 mmol, 1.0 eq.), DIEA (772.0 mg, 6.0 mmol, 3.0 eq.) and DCM (6 mL) at 0° C. The reaction mixture was stirred overnight at R.T, poured into ice water (5 mL) after the reaction was completed, and extracted with DCM (5 mL×2). The organic layers were combined, washed with brine (5 mL), dried over Na₂SO₄, and concentrated to afford a residue containing INT C20-2 (600.0 mg, 86.0%) as yellow oil. ¹H NMR (300 MHz, DMSO-d₆): δ 7.92 (d, J=8.4 Hz, 1H), 7.62-7.61 (m, 2H), 7.52 (t, J=1.2 Hz, 1H), 7.39 (d, J=2.1 Hz, 1H), 5.36 (s, 2H), 3.61 (s, 3H), 2.51 (s, 3H). LCMS: 351 ([M+H]⁺).

Pd(dppf)Cl₂ (125.3 mg, 0.2 mmol, 0.1 eq.) and KOAc (504.3 mg, 37.98 mmol, 3.0 eq.) were added to a mixture of INT C20-2 (600.0 mg, 1.7 mmol, 1.0 eq.), Bis(pinacolato)diborane (869.9 mg, 3.4 mmol, 2.0 eq.) and toluene (6 mL). The reaction mixture was stirred overnight at 110° C. under N₂ atmosphere, cooled to room temperature after the reaction was completed, and then filtered. The filtrate was concentrated under reduced pressure to obtain a residue which was purified with silica gel chromatography (eluting with Petroleum ether/EtOAc=5:1) to afford INT C20 (560.0 mg, 99.6%) as a yellow solid. ¹H NMR (300 MHz, CDCl₃): δ 8.40 (s, 1H), 7.75 (d, J=2.7 Hz, 1H), 7.65 (d, J=8.4, 1H), 7.45 (d, J=2.4, 1H), 7.28-7.25 (m, 1H), 5.28 (s, 2H), 3.51 (s, 3H), 2.51 (s, 3H), 1.42 (s, 12H). LCMS: 329.2 ([M+H]⁺).

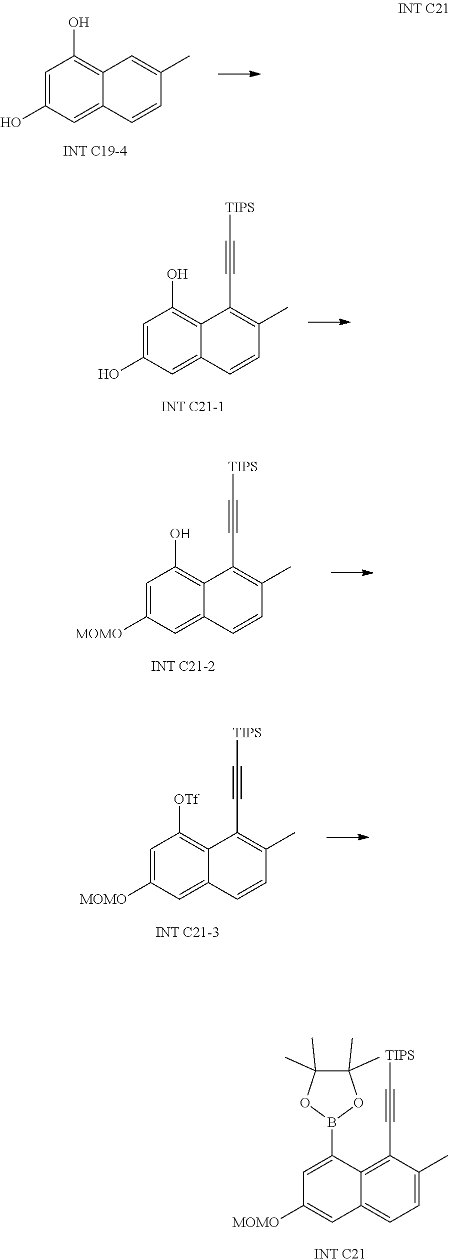

KOAc (1.7 g, 17.2 mmol, 2.0 eq.), (bromoethynyl)triisopropylsilane (2.4 g, 9.0 mmol, 1.05 eq.), INT C19-4 (1.5 g, 8.6 mmol, 1.0 eq.), t-BuOH (10.5 mL, 7.0 V), and Dichloro(p-cymene)ruthenium(II) dimer (2.6 g, 4.3 mmol, 0.5 eq.) were placed in a sealed vial. The reaction mixture was irradiated in the microwave at 160° C. for 2 hrs under the N₂ atmosphere, cooled to R.T and filtered through a pad of celite. The filtrate was concentrated to afford a crude product which was purified with silica gel chromatography (Petroleum ether/EtOAc=10:1) to afford INT C21-1 (430.3 mg, 14.1%) as yellow oil. H NMR (300 MHz, DMSO-d₆): δ 9.79 (s, 1H), 9.49 (s, 1H), 7.51 (d, J 8.4 Hz, 1H), 7.26 (d, J=8.4 Hz, 1H), 6.58-6.52 (m, 1H), 1.15-1.05 (m, 21H). LCMS: 355.2 ([M+H]⁺).

MOMCl (295.2 mg, 3.7 mmol, 1.3 eq.) was added dropwise to a mixture of INT C21-1 (1.0 g, 2.8 mmol, 1.0 eq.), DIEA (1.1 g, 8.5 mmol, 3.0 eq.) and DCM (10 mL) at 0° C. The reaction mixture was stirred overnight at R.T, poured into ice/water (10 mL) after the reaction was completed, and extracted with DCM (10 mL×2). The organic layers were combined, washed with brine (10 mL), dried over Na₂SO₄, and concentrated to afford a residue. The residue was purified with silica gel chromatography (eluting with petroleum ether) to afford INT C21-2 (500.0 mg, 44.6%) as a yellow solid. ¹H NMR (300 MHz, DMSO-d₆): δ 10.01 (s, 1H), 7.64 (d, J 8.4 Hz, 1H), 7.35 (d, J=8.4 Hz, 1H), 6.88 (d, J=2.4 Hz, 1H), 6.66 (d, J=2.4 Hz, 1H), 5.23 (s, 2H), 3.40 (s, 3H), 2.52 (s, 3H), 1.15 (s, 21H).

A mixture of C21-2 (500.0 mg, 1.3 mmol, 1.0 eq.), DIEA (504.0 mg, 3.9 mmol, 3.0 eq.) and DCM (8 mL) was cooled to −40° C., then Tf₂O (550.2 g, 2.0 mmol, 1.5 eq.) was added. The reaction mixture was stirred at −40° C. for 30 mins, quenched with water (5 mL) after the reaction was completed, and extracted with DCM (5 mL×2). The organic layers were combined, washed with brine (5 mL×1), dried over Na₂SO₄, and concentrated to obtain a crude product which was purified with silica gel chromatography (Petroleum ether/EtOAc=30:1) to afford INT C21-3 (501.0 mg, 72.6%) as yellow oil. ¹H NMR (300 MHz, DMSO-d₆): δ 7.91 (d, J 8.4 Hz, 1H), 7.65 (d, J=2.4 Hz, 1H), 7.57 (d, J=8.4 Hz, 1H), 7.38 (d, J=2.1 Hz, 1H), 5.34 (s, 2H), 3.41 (s, 3H), 2.59 (s, 3H), 1.14 (s, 21H).

Pd(dppf)Cl₂ (58.5 mg, 0.08 mmol, 0.1 eq.) and KOAc (220.8 mg, 2.25 mmol, 3.0 eq.) were added to a mixture of INT C21-3 (400.0 mg, 0.75 mmol, 1.0 eq.), Bis(pinacolato)diborane (382.8 mg, 1.51 mmol, 2.0 eq.) and toluene (4 mL). The reaction mixture was stirred at 110° C. for 3 hrs under N₂ atmosphere, cooled to room temperature after the reaction was completed, filtered and concentrated under reduced pressure to obtain a residue. The residue was purified with silica gel chromatography (eluting with petroleum ether/EtOAc=30:1) to afford INT C21 (202.0 mg, 53.0%) as a yellow solid. ¹H NMR (300 MHz, CDCl₃): δ 7.59 (d, J 8.4 Hz, 1H), 7.46 (d, J=1.8 Hz, 1H), 7.33-7.27 (m, 2H), 5.26 (s, 2H), 3.50 (s, 3H), 2.62 (s, 3H), 1.58 (s, 12H), 1.18 (s, 21H). LCMS: 509.2 ([M+H]⁺).

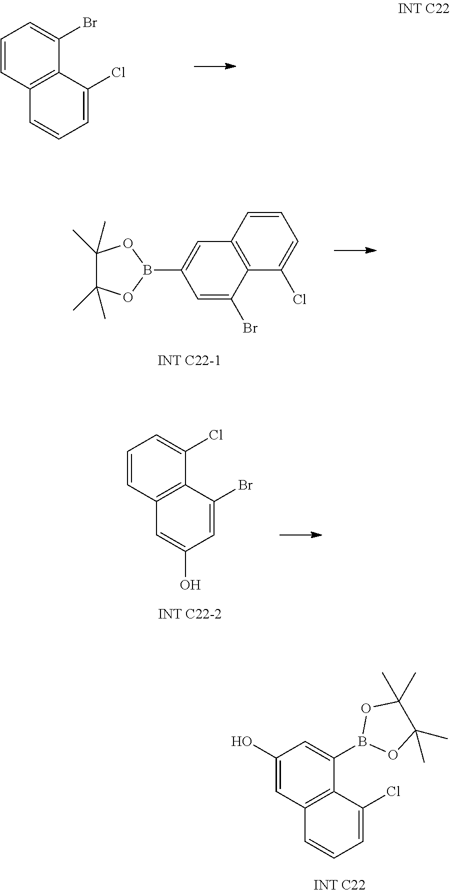

A mixture of 1-bromo-8-chloronaphthalene (8.0 g, 33.1 mmol, 1.0 eq), pinacolborane (10.6 g, 82.8 mmol, 2.5 eq), dtbbpy (1.1 g, 4.0 mmol, 0.12 eq), [Ir(OMe)(1,5-cod)]₂ (2.2 g, 3.3 mmol, 0.1 eq) and THF (130 mL) was stirred overnight at 60° C. under argon atmosphere, and then concentrated after the reaction was completed to obtain a residue. The residue was purified with gel column chromatography (eluting with petroleum ether) to afford a crude product containing INT C22-1 (10.0 g) as a white solid which was used for next step without further purification.

AcOH (31.6 g, 525.76 mmol, 69.0 eq) and aq. H₂O₂(30%, 7.8 g, 68.58 mmol, 9.0 eq) were added to a solution of the crude product containing INT C22-1 (2.8 g) in a mixed solution of THF and H₂O (V_{THF}:V_{H2O}=3:1, 21.2 mL). The reaction mixture was stirred at R.T for 1 h, and then saturated aq. NaHSO₃ (120 mL) was added. The resulting mixture was stirred at R.T for 1 h, and then extracted with EtOAc (100 mL×3). The organic layers were combined, and concentrated to obtain a residue which was purified with gel column chromatography (eluting with petroleum ether/ethyl acetate=80:1 to 20:1) to afford a crude product containing INT C22-2 (620 mg) as a white solid.

A mixture of the crude product containing INT C22-2 (2.0 g, 7.8 mmol, 1.0 eq), Bis(pinacolato)diborane (4.0 g, 15.5 mmol, 2.0 eq), KOAc (2.4 g, 23.3 mmol, 3.0 eq), Pd(dppf)Cl₂ (1.7 g, 2.3 mmol, 0.3 eq) and toluene (80 mL) was stirred at 80° C. for 5 hrs under N₂ atmosphere, cooled to R.T, and then water (80 mL) was added. The resulting mixture was extracted with EtOAc (80 mL×3) and the organic layers were combined, washed with saturated brine, dried over anhydrous Na₂SO₄, and then concentrated to afford a residue which was purified with gel column chromatography (eluting with petroleum ether/ethyl acetate=20:1) to obtain a crude product which was purified with pre-HPLC to afford INT C22 (680 mg, 2.2 mmol, 28% yield) as a white solid. ¹H NMR (300 MHz, DMSO-d₆): δ 9.98 (s, 1H), 7.71 (dd, J=7.9, 1.6 Hz, 1H), 7.55-7.27 (m, 2H), 7.21 (d, J=2.7 Hz, 1H), 7.16 (d, J=2.7 Hz, 1H), 1.35 (s, 12H). LCMS: 305.1 ([M+H]⁺).

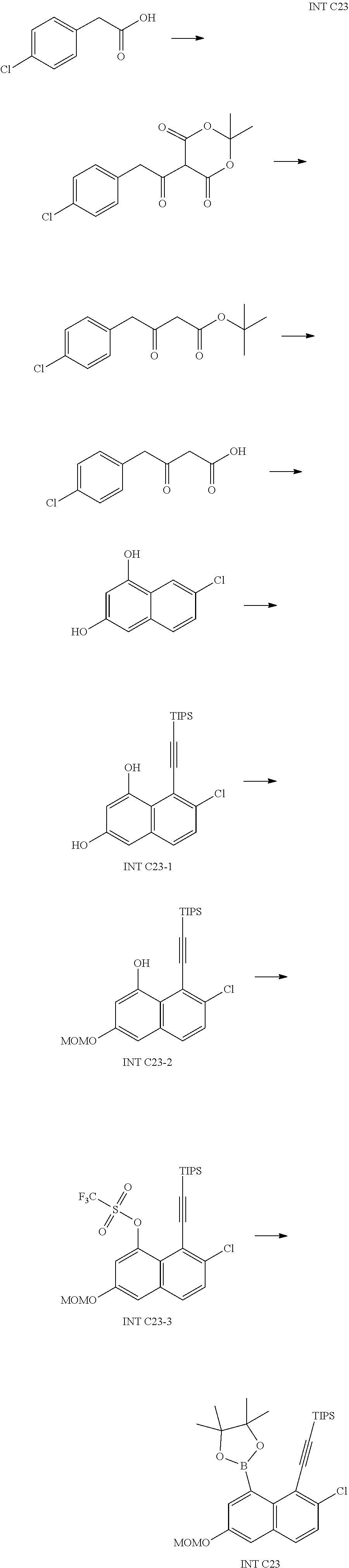

Following an analogous procedure described in procedure of INT C19, 7-chloronaphthalene-1,3-diol was obtained with 2-(4-chlorophenyl)acetic acid as start material.

Bis(dichloro(-6-p-cymene)ruthenium) (0.41 g, 0.67 mmol) and potassium acetate (1.63 g, 16.61 mmol) were added to a solution of 7-chloronaphthalene-1,3-diol (1.23 g, 6.32 mmol) and (bromoethynyl)triisopropylsilane (1.94 g, 7.43 mmol) in 1,4-dioxane (20 mL) at R.T. The reaction mixture was purged with nitrogen, stirred at 100° C. for 18 hrs, cooled to R.T, and then filtered. The filtrate was concentrated under reduced pressure to obtain a residue which was purified with silica gel chromatography (EA:Hex=1:25, v/v) to afford INT C23-1 (1.77 g, 74.7%).

Bromomethyl methyl ether (0.79 g, 6.32 mmol) was added dropwise to a solution of INT C23-1 (1.77 g, 4.72 mmol) and DIEA (2.03 g, 15.71 mmol) in DCM (20 mL) at 0° C. The reaction mixture was stirred at 0° C. for 0.5 h, and then quenched with water (50 mL). The organic layer was concentrated under reduced pressure to afford a crude product containing INT C23-2 (1.97 g) which was used to next step directly without further purification.

Tf₂O (2.04 g, 7.23 mmol) was added dropwise to a solution of the crude product containing INT C23-2 (1.97 g) and DIEA (1.91 g, 14.81 mmol) in DCM (80 mL) at −45° C. The reaction mixture was stirred for 3 hrs at −45° C., warmed to R.T and quenched with water (10 mL). The organic layer was concentrated under reduced pressure to obtain a residue which was purified with silica gel chromatography (EA:Hex=1:20-1:10, v/v) to afford INT C23-3 (1.94 g, the yield of the above two steps is 74.88%).

A mixture of INT C23-3 (1.98 g, 3.59 mmol), 4,4,4′,4′,5,5,5′,5′-Octamethyl-2,2′-bi(1,3,2-dioxaborolane) (1.52 g, 5.99 mmol), Pd(dppf)Cl₂ (0.30 g, 0.41 mmol), potassium acetate (1.30 g, 13.25 mmol) and toluene (20 mL) was purged with nitrogen, stirred at 110° C. for 3.5 hrs, cooled to R.T, and then filtered. The filtrate was concentrated under reduced pressure to obtain a residue which was purified with silica gel chromatography (EA:Hex=1:0-10:1, v/v) to afford INT C23 (232 mg, 12.2%).

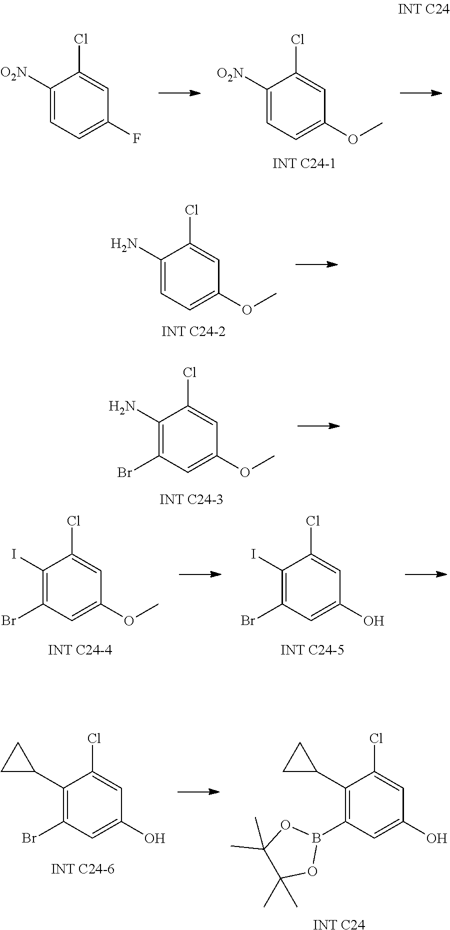

2-chloro-4-fluoro-1-nitrobenzene (25.0 g, 142.4 mmol, 1.0 eq) was added dropwise to a solution of NaOMe (9.3 g, 171 mmol, 1.2 eq) dissolved MeOH (100 mL) when the temperature was below 0° C. The reaction mixture was stirred at R.T for 4 hrs, poured into ice/water (100 mL) to precipitate the solid. The solid was collect by filtration and washed with water (20 mL×2) to afford INT C24-1 (30.4 g) as a white solid. ¹H NMR (300 MHz, DMSO-d₆): δ 8.11 (d, J=9.0 Hz, 1H), 7.33 (d, J=2.7 Hz, 1H), 7.11 (dd, J=9.3, 2.7 Hz, 1H), 3.91 (s, 3H). LCMS: m/z=188.0 [M+H]⁺.

A mixture of INT C24-1 (30.4 g, 162 mmol, 1.0 eq), NH₄Cl (34.7 g, 648 mmol, 4.0 eq), Fe (90.7 g, 1.6 mol, 10.0 eq), MeOH (300 mL) and water (100 mL) was stirred for 6 hrs at 50° C., cooled to R.T, and then filtered. The filter cake was washed with EtOAc (50 mL×3), and the filtrate was washed with water (100 mL) and brine (50 mL) successively, dried with anhydrous Na₂SO₄, and then concentrated to afford a residue containing INT C24-2 which was used for next step without further purification. LCMS: m/z=158.1 [M+H]⁺.

Br₂ (27.0 g, 16 mmol, 0.9 eq) was added dropwise to a solution of the residue containing INT C24-2 (28.2 g, 18 mmol, 1.0 eq) in DCM (60 mL) when the temperature was below 0° C. The reaction mixture was stirred at 25° C. for 4 hrs, then quenched with aq. Na₂S₂O₃ (30 mL), extracted with EtOAc (50 mL×2). The organic layers were combined, washed with water (30 mL) and brine (30 mL) successively, dried with anhydrous Na₂SO₄, and concentrated to afford a residue which was purified by silica gel chromatography (eluting with petroleum ether/EtOAc=100:1) to afford INT C24-3 (20.7 g, yield 48.9%) as a yellow solid.

A solution of NaNO₂ (12.0 g, 175 mmol, 2.0 eq) and KI (36.3 g, 218.8 mmol, 2.5 eq) in water (100 mL) were added to the solution of INT C24-3 (20.7 g, 87.5 mmol, 1.0 eq) and p-Toluenesulfonic acid monohydrate (50.5 g, 262.5 mmol, 3.0 eq) in CH₃CN (600 mL) when the temperature was below 0° C. The reaction mixture was stirred at R.T for 5 hrs, then quenched with aq. NaHCO₃ and aqueous Na₂S₂O₃ successively. The resulting mixture was stirred for 1 h, extracted with EtOAc (50 mL×2). The organic layers were combined, washed with brine (20 mL), dried with anhydrous Na₂SO₄, and concentrated to obtain a residue which was purified with reverse phase C18 column chromatography (eluted with CH₃CN/water=3:1) to afford INT C24-4 (10.5 g, yield 34.5%) as a red solid. ¹H NMR (300 MHz, DMSO-d₆): δ 7.33 (d, J=3.0 Hz, 1H), 7.23 (d, J=2.7 Hz, 1H), 3.79 (s, 3H).

BBr₃ (21.0 g, 85 mmol, 3.6 eq) was added dropwise to a solution of INT C24-4 (8.2 g, 23.6 mmol, 1.0 eq) in DCM (80 mL) at 0° C. The reaction mixture was stirred for 4 hrs at R.T, then quenched with water (50 ml). The pH of the resulting mixture was adjusted to 8-9 with aq. NaOH, and then extracted with DCM (20 mL). The organic layer was washed with water (20 mL) and brine (10 mL) successively, dried with anhydrous Na₂SO₄, and concentrated to obtain a residue. The residue was slurried with hexane, and INT C24-5 (4.6 g, yield 58.4%) was collected by filtration as a red solid. ¹H NMR (300 MHz, DMSO-d₆): δ 7.07 (d, J=3.6 Hz, 1H), 6.94 (d, J=2.7 Hz, 1H). LCMS: m/z=330.8, 332.8 [M−H]⁻.

A mixture of INT C24-5 (4.1 g, 12.2 mmol, 1.0 eq), Cyclopropylboronic acid (1.6 g, 18.3 mmol, 1.5 eq), K₃PO₄ (9.3 g, 44.0 mmol, 3.6 eq), Pd(dppf)Cl₂ (446 mg, 0.6 mmol, 0.05 eq), a mixed solution of 1,4-Dioxane and water (Dioxane/water=3:1, 40 mL) was purged with argon for 10 mins, stirred for 13 hrs at 110° C. under argon atmosphere, cooled to R.T, poured into water (10 mL) and extracted with EtOAc (10 mL×3). The organic layers were combined, washed with water (10 mL) and brine (10 mL) successively, dried with anhydrous Na₂SO₄, and concentrated to obtain a residue which was purified with silica gel chromatography (eluting with petroleum ether/EtOAc=30:1) to afford INT C24-6 (1.5 g) as a gray solid. LCMS: m/z=245.0, 247.1 [M−H]⁻.

INT C24-6 (1.5 g, 6.1 mmol, 1.0 eq), Bis(pinacolato)diborane (3.9 g, 15.3 mmol, 2.5 eq), AcOK (1.78 g, 18.2 mmol, 3.0 eq), Pd(dppf)Cl₂ (444 mg, 0.6 mmol, 0.1 eq) were added successively into 1,4-dioxane (15 mL). The reaction was purged with argon for 10 mins, stirred for 13 hrs at 110° C. under argon, cooled to R.T, poured into water (10 mL) and extracted with EtOAc (10 mL×3). The organic layers were combined, washed with water (10 mL) and brine (10 mL) successively, dried with anhydrous Na₂SO₄, and concentrated to obtain a residue which was purified with Prep-HPLC to afford INT C24 (100 mg) as a gray solid. LCMS: m/z=295.3 [M+H]⁺.

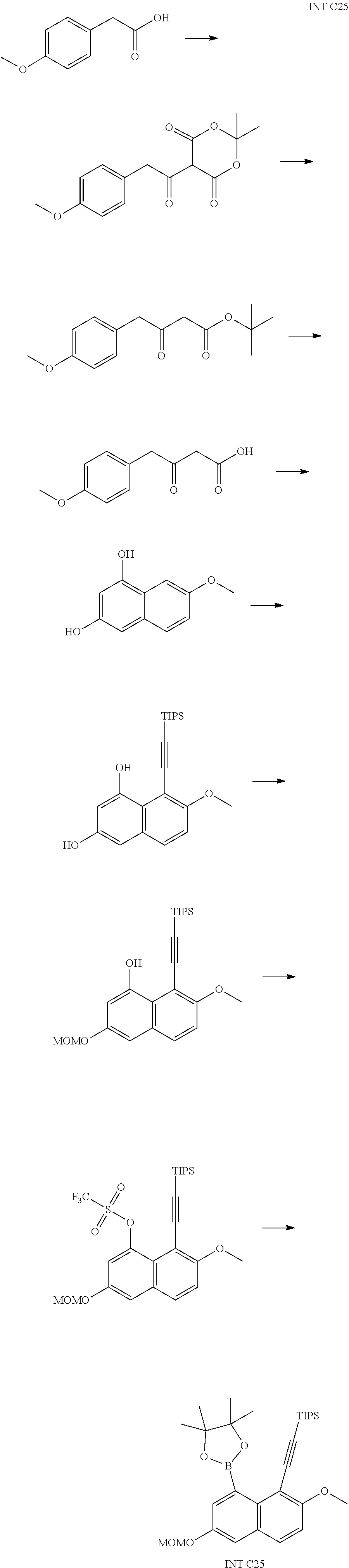

Following an analogous procedure described in procedure of INT C23, INT C25 was obtained with 2-(4-methoxyphenyl)acetic acid as start material.

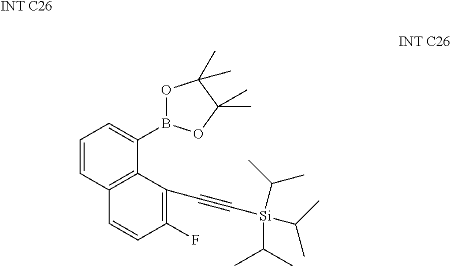 [SMILES: "INT C26" ; CC(C)[Si](C#Cc1c(F)ccc2cccc(B3OC(C)(C)C(C)(C)O3)c12)(C(C)C)C(C)C]

INT C26 was synthesized with 7-fluoronaphthalen-1-ol and INT C15-1 as starting materials through using conventional preparation method.

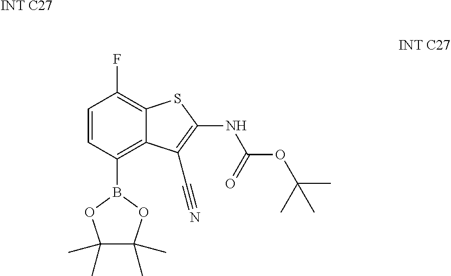 [SMILES: "INT C27" ; CC(C)(C)OC(=O)Nc1sc2c(F)ccc(B3OC(C)(C)C(C)(C)O3)c2c1C#N]

INT C27 was synthesized through using conventional preparation method.

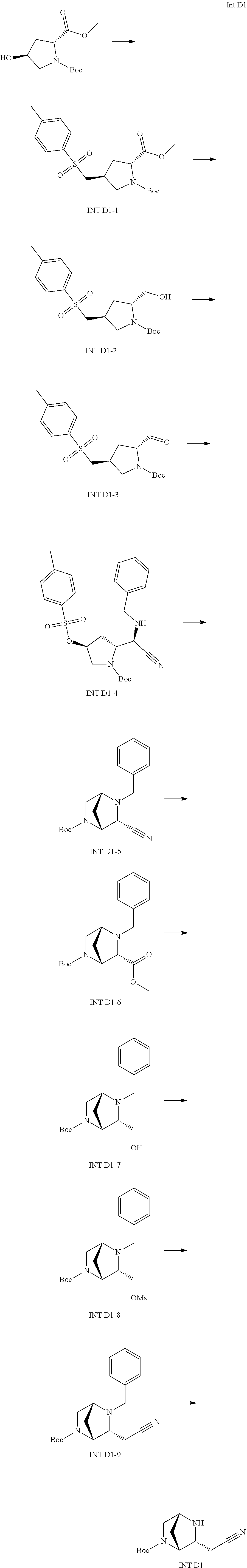

4-methylbenzenesulfonyl chloride (35.56 g, 186.52 mmol) was added dropwise to a solution of 1-(tert-butyl) 2-methyl (2R,4S)-4-hydroxypyrrolidine-1,2-dicarboxylate (16.3 g, 66.46 mmol) in pyridine (160 mL) at 0° C. The reaction mixture was stirred for 22 hrs at R.T, and then concentrated under reduced pressure to obtain a residue. A mixture of the residue and EA (300 mL) was washed with hydrochloric acid solution (150 mL, 0.5 M), saturated ammonium chloride solution (2×200 mL) and brine (200 mL) successively. The organic phase was dried over anhydrous Na₂SO₄, concentrated under reduced pressure to afford a crude product containing INT D1-1 (45.43 g). LCMS: m/z=400 [M+H]⁺.

LiBH₄ (2 M/THF, 125 mL) was added dropwise to a solution of INT D1-1 (45.43 g) in THF (100 mL) at 0° C. The reaction mixture was stirred for 3 hrs at R.T, quenched with water (20 mL) and CH₃COOH (4 mL), and then diluted with EA (200 mL). The resulting mixture was washed with saturated sodium bicarbonate solution (200 mL) and brine (2×200 mL) successively, and the organic layer was concentrated under reduced pressure to obtain a residue which was purified with silica gel chromatography (DCM:MeOH=100:1, v/v) to afford INT D1-2 (22.18 g, 52.5%). LCMS: m/z=372 [M+H]⁺.

DMSO (7.71 g, 98.68 mmol), INT D1-2 (22.18, 59.71 mmol) and TEA (18.35 g, 181.34 mmol) were added dropwise successively to a solution of oxalyl chloride (11.43 g, 90.05 mmol) in DCM (350 mL) at −78° C. The reaction mixture was stirred for 1 h at 0° C., then quenched with water (50 mL). The resulting mixture was washed with aq. hydrochloric acid (100 mL, 1 M), saturated sodium bicarbonate solution (150 mL) and brine (150 mL) successively. The organic phase was separated, dried over Na₂SO₄, and concentrated under reduced pressure to obtain a residue which was purified with silica gel chromatography (Hex:EA=5:1, v/v) to afford INT D1-3 (16.35 g, 74.1%). LCMS: m/z=370 [M+H]⁺.

Diethyl cyanophosphonate (8.76 g, 53.71 mmol) and phenylmethanamine (11.57 g, 107.98 mmol) were added dropwise to a solution of INT D1-3 (15.9 g, 43.04 mmol) dissolve in THF (200 mL). The reaction mixture was stirred for 1 h at R.T, diluted with EA (200 mL) and washed with brine (2×300 mL). The organic layer was separated and concentrated under reduced pressure to obtain a residue which was purified with silica gel chromatography (Hex:EA=8:1, v/v) to afford INT D1-4 (14.85 g, 71.1%). LCMS: m/z=486 [M+H]⁺.

A mixture of INT D1-4 (14.85 g, 30.58 mmol), DIEA (3.95 g, 30.58 mmol) and DCM (230 mL) was stirred for 46.5 hrs at 100° C., cooled to room temperature, and then concentrated under reduced pressure to obtain a residue. A mixture of residue and EA (200 mL) was washed with brine (2×200 mL), and the organic layer was collected and concentrated under reduced pressure to obtain a residue which was purified with silica gel chromatography (Hex:EA=10:1, v/v) to afford INT D1-5 (3.54 g, 36.9%). LCMS: m/z=314 [M+H]⁺.

INT D1-5 (3.33 g, 10.63 mmol) was added to a solution of NaOMe (2.87 g, 53.18 mmol) in MeOH (106 mL). The reaction mixture was stirred at 60° C. for 6 hrs and then stirred at R.T for 18 hrs, cooled to 0° C., and then quenched with aq. hydrochloric acid (3 M, 35 mL). After the pH of the resulting mixture was adjusted to 8 with NaHCO₃, the resulting mixture was concentrated under reduced pressure to remove MeOH, and then extracted with DCM (2×100 mL). The organic layers were combined washed with brine (2×100 mL), dried over Na₂SO₄ and concentrated under reduced pressure to obtain a residue which was purified with silica gel chromatography (Hex:EA=10:1-7:1, v/v) to afford INT D1-6 (2.20 g, 59.8%). LCMS: m/z=347 [M+H]⁺.

LiBH₄ (2 M/THF, 8 mL) was added dropwise to a solution of INT D1-6 (2.20 g, 6.35 mmol) in THF (40 mL) at 0° C. The reaction mixture was stirred for 19 hrs at R.T, and then quenched with water (10 mL) and CH₃COOH (2 mL). The resulting mixture was diluted with EA (100 mL) and washed with saturated sodium bicarbonate solution (100 mL) and brine (100 mL) successively, dried over Na₂SO₄ and concentrated under reduced pressure to obtain a residue which was purified with silica gel chromatography (DCM:MeOH=50:1-30:1, v/v) to afford INT D1-7 (1.51 g, 74.7%). LCMS: m/z=319 [M+H]⁺.

Methanesulfonyl chloride (1.03 g, 8.99 mmol) was added dropwise to a solution of INT D1-7 (1.51 g, 4.74 mmol) and TEA (1.12 g, 11.07 mmol) in THF (16 mL). The reaction mixture was stirred at R.T for 3 hrs, then diluted with EA (30 mL) and washed with brine (3×30 mL). The organic layer was separated and concentrated under reduced pressure to afford a crude product containing INT D1-8 (1.90 g). LCMS: m/z=397 [M+H]⁺.

TMSCN (2.17 g, 21.87 mmol) was added to a mixture of the crude product containing INT D1-8 (1.90 g, 4.79 mmol), CsF (2.99 g, 19.68 mmol) and DMF (19 mL). The reaction mixture was stirred at 60° C. for 2.5 hrs, cooled to R.T and diluted with EA (30 mL). The resulting mixture was washed with brine (3×30 mL). The organic layer was separated and concentrated under reduced pressure to obtain a residue which was purified with silica gel chromatography (EA:Hex=1:2, v/v) to afford INT D1-9 (1.57 g, 67.2%). LCMS: m/z=328 [M+H]⁺.

Wet Pd/C (570 mg, 10%) was added to a solution of INT D1-9 (325 mg, 0.99 mmol) in MeOH (5 mL). The reaction mixture was stirred at R.T for 70 mins under hydrogen atmosphere, and then filtered. The filtrate was concentrated under reduced pressure to afford INT D1 (221 mg, 93.8%). LCMS: m/z=238 [M+H]⁺.

### Example 1

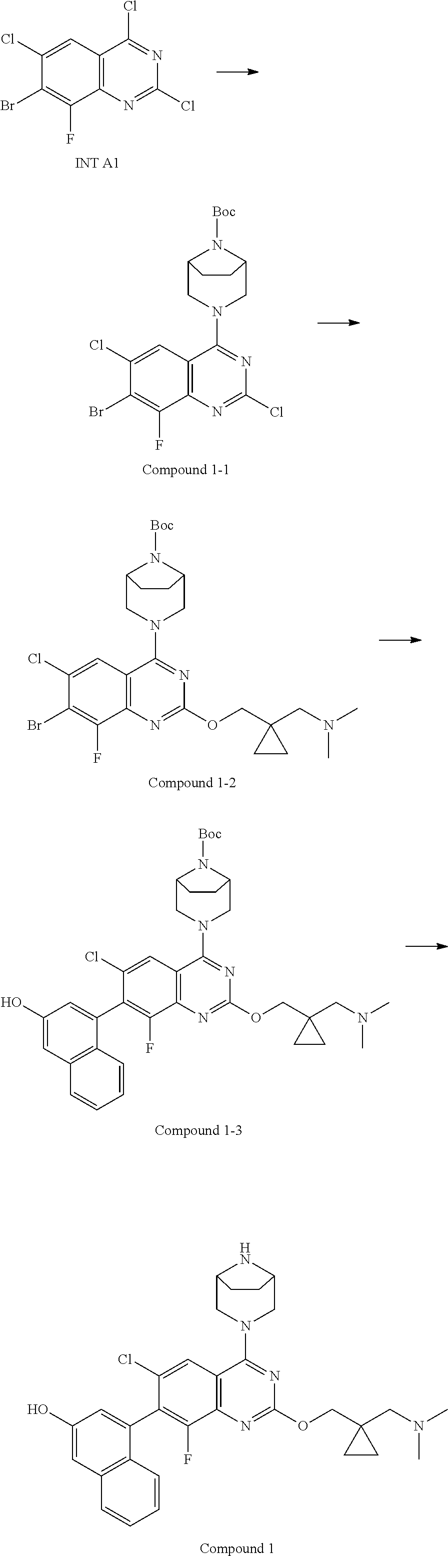

A solution of TNT A1 (300 mg, 0.9 mmol), Et₃N (273 mg, 2.7 mmol) and tert-butyl (1R,5S)-3,8-diazabicyclo[3.2.1]octane-8-carboxylate (191 mg, 0.9 mmol) in 1,4-dioxane (3 mL) was stirred at room temperature for 0.5 h. The solution was concentrated under vacuum, the residue was dissolved in DCM (200 mL), washed with brine (2×100 mL), dried over Na₂SO₄ and concentrated in vacuum. The residue was purified by Prep-TLC (EA/Hex=1:5, v/v) to afford the desired product Compound 1-1(320 mg) as an off-white solid. MS m/z: 505 [M+H]⁺.

A solution of Compound 1-1 (270 mg, 0.53 mmol), INT B1 (343 mg, 2.65 mmol), KF (62 g, 1.06 mmol) in DMSO (5 mL) was stirred at 90° C. for 4 h under nitrogen atmosphere. The mixture was allowed to cool to room temperature and partitioned between water and EA. The organic layer was washed with brine, dried over Na₂SO₄ and concentrated in vacuum. The residue was purified by Prep-TLC (MeOH:DCM=1:10, v/v) to afford the desired product Compound 1-2 (300 mg). MS m/z: 598 [M+H]⁺.

To a solution of Compound 1-2 (59.9 mg, 0.1 mmol), INT C1 (91.09 mg, 0.3 mmol) in 1,4-dioxane (2 mL) and water (0.5 mL) was added Na₂CO₃ (32 mg, 0.3 mmol) and Pd(PPh₃)₄(12 mg, 0.01 mmol). The reaction mixture was stirred at 100° C. for 2 hours under nitrogen atmosphere. The reaction was diluted with EA (30 mL) and washed with brine. The organic layer was dried over Na₂SO₄ and concentrated under vacuum. The residue was purified by Pre-TLC to afford Compound 1-3 (20 mg). MS m/z: 662 [M+H]⁺.

A solution of Compound 1-3 (20 mg, 0.03 mmol), TFA (0.5 mL) in DCM (2 mL) was stirred at RT for 0.5 h. The mixture was concentrated in vacuum. The residue was diluted with DCM (30 mL) and washed with sat.aq.NaHCO₃ (2×30 mL). The organic layer was dried over MgSO₄ and concentrated under vacuum to afford the desired product (25 mg). MS m/z: 562 [M+H]⁺.

¹H NMR (400 MHz, d₆-DMSO) δ 8.00 (s, 1H), 7.82 (d, J=8.4 Hz, 1H), 7.47-7.45 (m, 1H), 7.30-7.29 (m, 1H), 7.24-7.15 (m, 2H), 7.05-7.04 (m, 1H), 4.55-4.51 (m, 2H), 4.32-4.29 (m, 2H), 4.25-4.20 (m, 2H), 3.80-3.77 (m, 2H), 3.21-3.20 (m, 2H), 2.50 (s, 6H), 1.96-1.96 (m, 4H), 0.86-0.78 (in, 4H).

The following compounds were synthesized using the above procedure or modified procedure with the corresponding starting materials.

| | | MS: | |
|---|---|---|---|
| Compound | Structure | [M + H]⁺ | ¹H NMR |
| 2 | 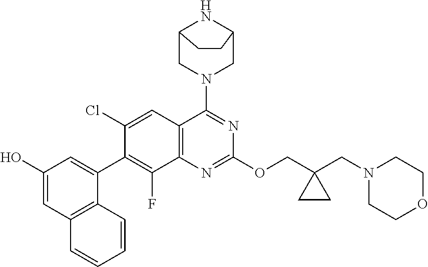 [SMILES: Oc1cc(-c2c(Cl)cc3c(N4CC5CCC(C4)N5)nc(OCC4(CN5CCOCC5)CC4)nc3c2F)c2ccccc2c1] | 604 | |
| 3 | 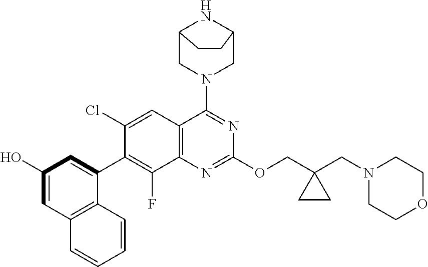 | 604 | |
| 4 | 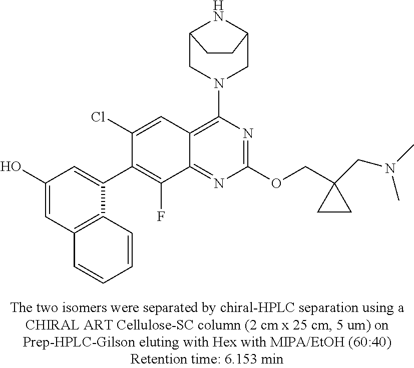 | 562 | |
| 5 | 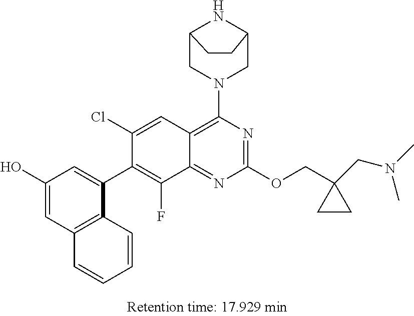 | 562 | |
| 6 | 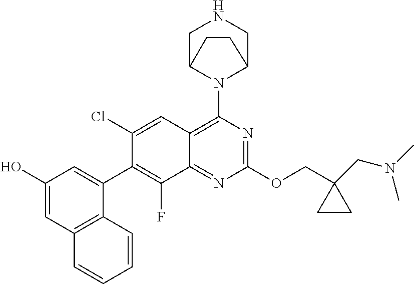 [SMILES: CN(C)CC1(COc2nc(N3C4CCC3CNC4)c3cc(Cl)c(-c4cc(O)cc5ccccc45)c(F)c3n2)CC1] | 562 | ¹H NMR (400 MHz, d₆-DMSO) δ 8.08 (s, 1H), 7.82 (d, J = 8.4 Hz, 1H), 7.47-7.45 (m, 1H), 7.31-7.30 (m, 1H), 7.25-7.21 (m, 2H), 7.07-7.06 (m, 1H), 5.05 (s, 2H), 4.30 (s, 2H), 3.51- 3.37 (m, 2H), 3.34-3.21 (m, 2H), 3.28-3.19 (m, 2H), 2.86- 2.84 (m, 6H), 2.16-2.07 (m, 4H), 0.88-0.85 (m, 2H), 0.79- 0.78 (m, 2H). |
| 7 | 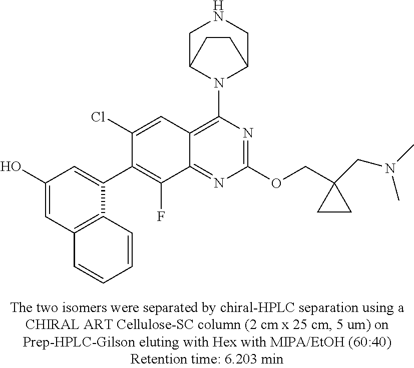 | 562 | |
| 8 | 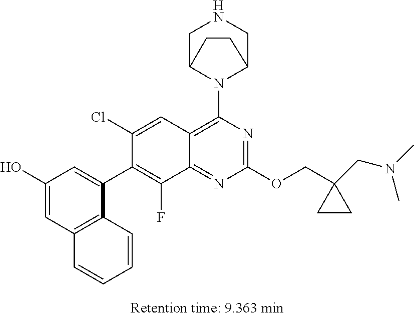 | 562 | |
| 9 | 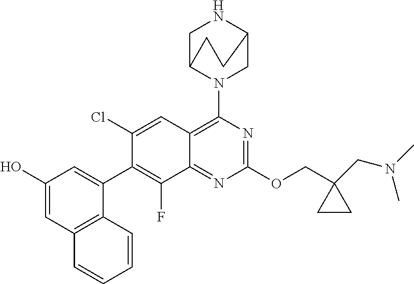 [SMILES: CN(C)CC1(COc2nc(N3CC4CCC3CN4)c3cc(Cl)c(-c4cc(O)cc5ccccc45)c(F)c3n2)CC1] | 562 | |
| 10 | 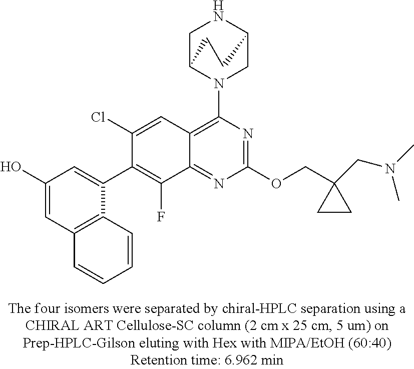 | 562 | |
| 11 | 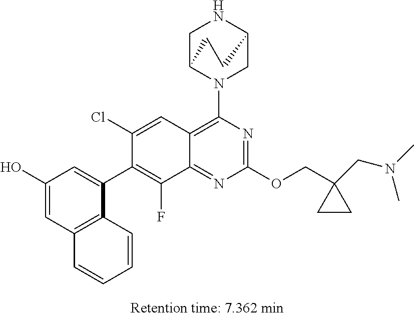 | 562 | |
| 12 | 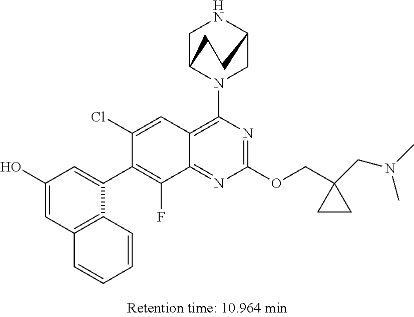 | 562 | |
| 13 | 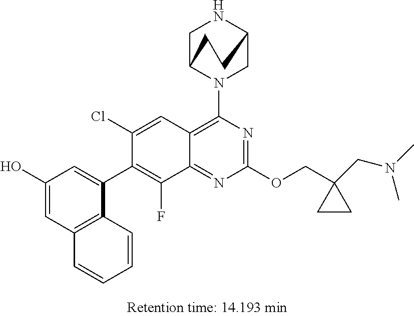 | 562 | |
| 14 | 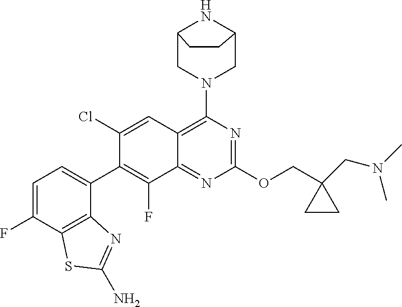 [SMILES: CN(C)CC1(COc2nc(N3CC4CCC(C3)N4)c3cc(Cl)c(-c4ccc(F)c5sc(N)nc45)c(F)c3n2)CC1] | 586 | |
| 15 | 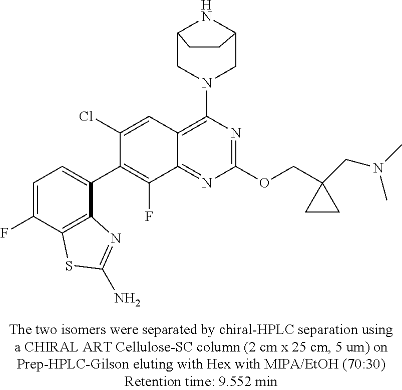 | 586 | |
| 16 | 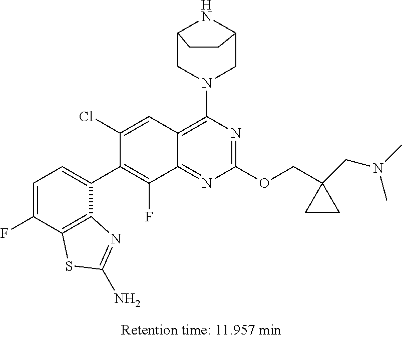 | 586 | |
| 17 | 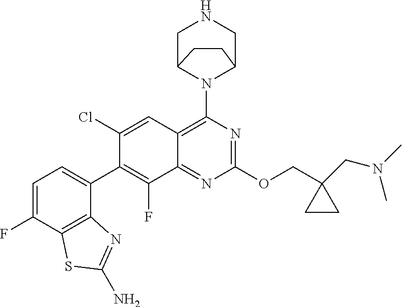 [SMILES: CN(C)CC1(COc2nc(N3C4CCC3CNC4)c3cc(Cl)c(-c4ccc(F)c5sc(N)nc45)c(F)c3n2)CC1] | 586 | |
| 18 | 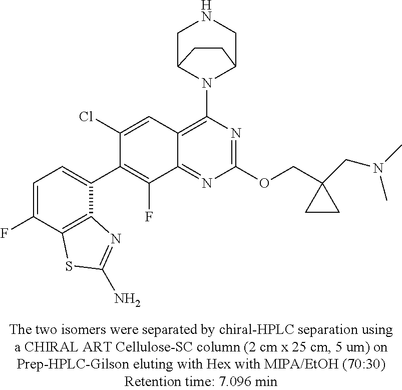 | 586 | |
| 19 | 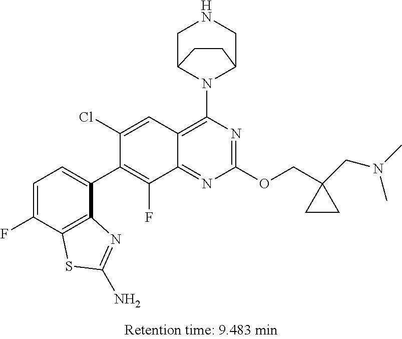 | 586 | |
| 20 | 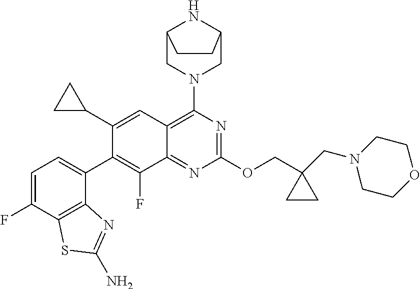 [SMILES: Nc1nc2c(-c3c(C4CC4)cc4c(N5CC6CCC(C5)N6)nc(OCC5(CN6CCOCC6)CC5)nc4c3F)ccc(F)c2s1] | 634 | |
| 21 | 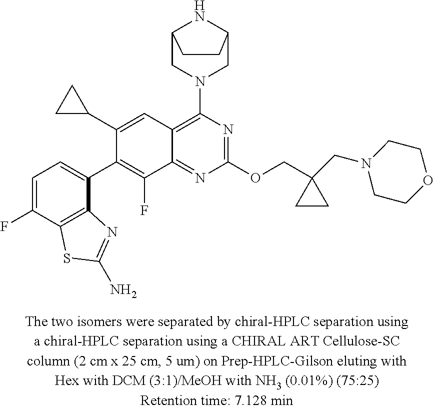 | 634 | |
| 22 | 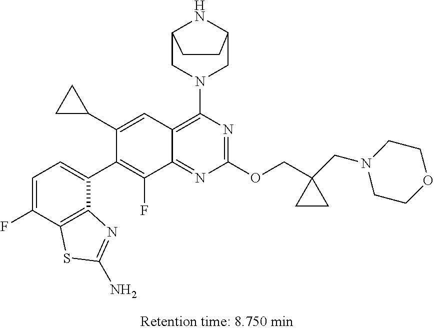 | 634 | |
| 23 | 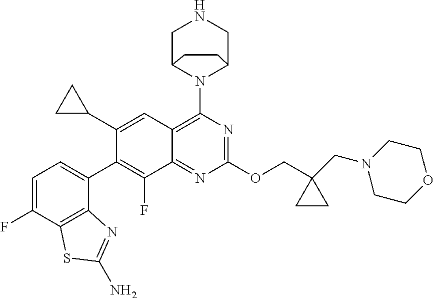 [SMILES: Nc1nc2c(-c3c(C4CC4)cc4c(N5C6CCC5CNC6)nc(OCC5(CN6CCOCC6)CC5)nc4c3F)ccc(F)c2s1] | 634 | |
| 24 | 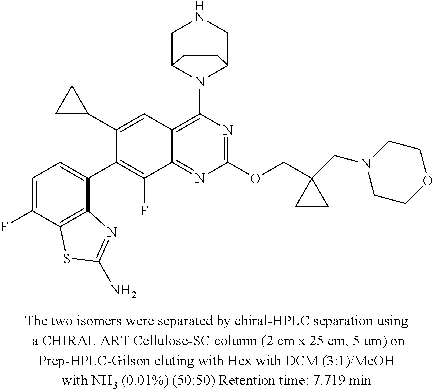 | 634 | |
| 25 | 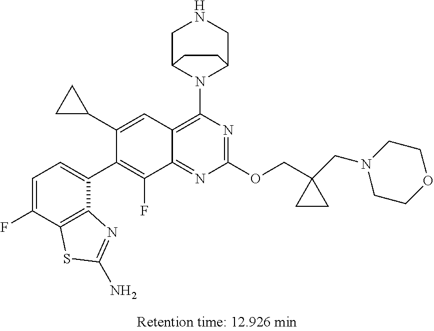 | 634 | |
| 26 | 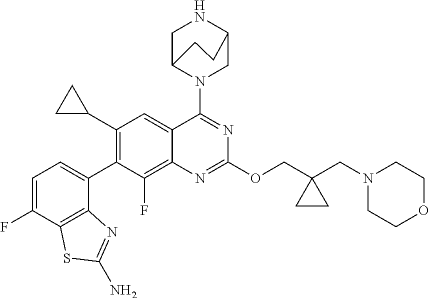 [SMILES: Nc1nc2c(-c3c(C4CC4)cc4c(N5CC6CCC5CN6)nc(OCC5(CN6CCOCC6)CC5)nc4c3F)ccc(F)c2s1] | 634 | |
| 27 | 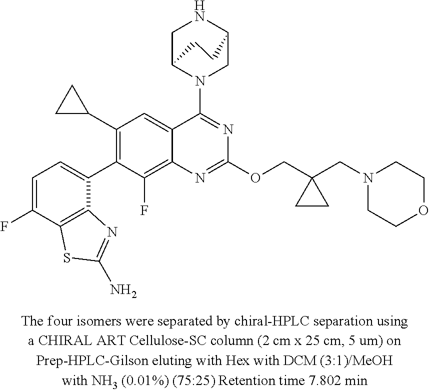 | 634 | |
| 28 | 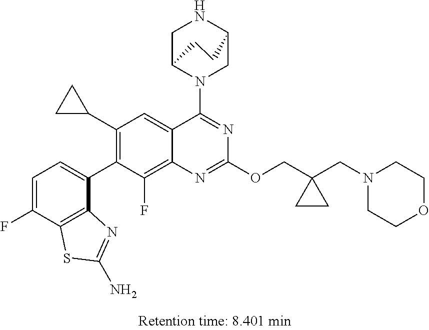 | 634 | |
| 29 | 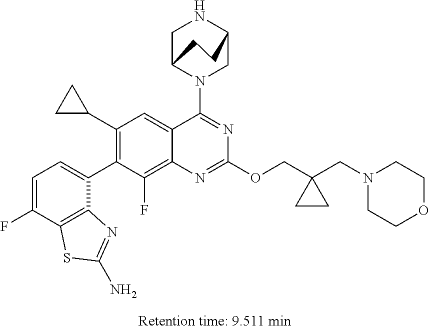 | 634 | |
| 30 | 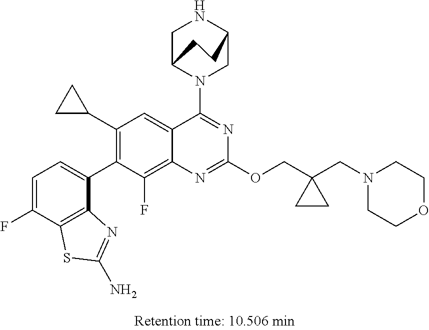 | 634 | |
| 31 | 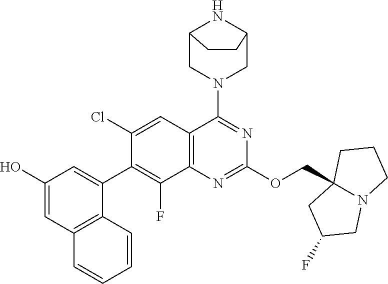 [SMILES: Oc1cc(-c2c(Cl)cc3c(N4CC5CCC(C4)N5)nc(OC[C@@]45CCCN4C[C@H](F)C5)nc3c2F)c2ccccc2c1] | 592 | |
| 32 | 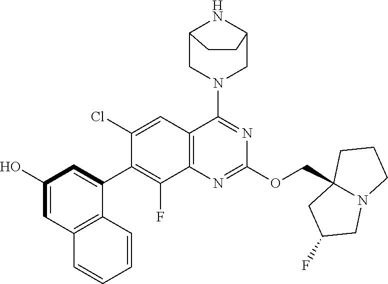 | 592 | |

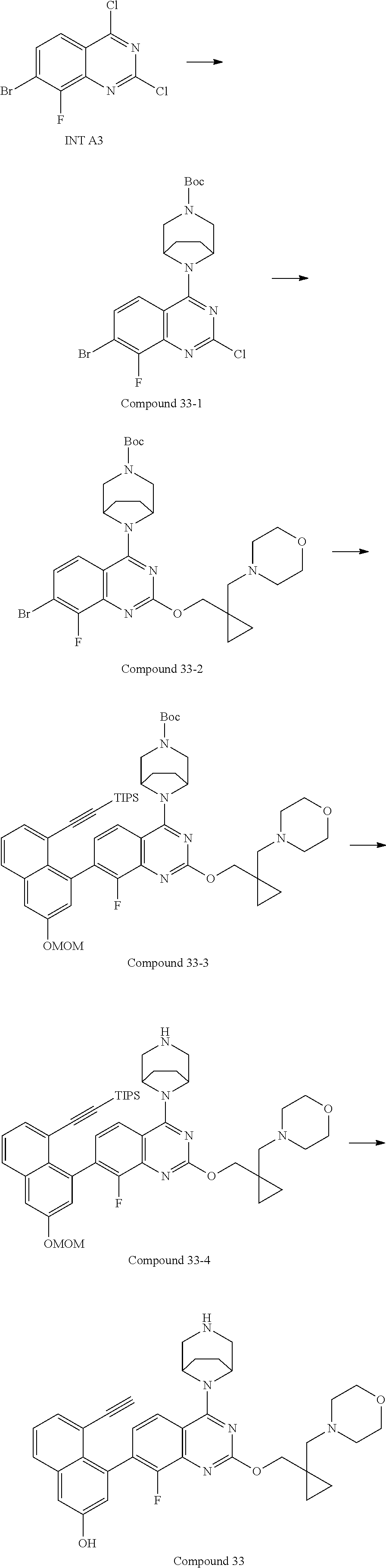

To a solution of INT A3 (7.08 g, 23.93 mmol) and tert-butyl (1R,5S)-3,8-diazabicyclo[3.2.1]octane-3-carboxylate (4.93 g, 23.22 mmol) in DCM (100 mL) was added DIEA (5.98 g, 46.27 mmol) at room temperature. The reaction mixture was stirred for 50 min, then water (50 mL) was added. The organic phase was collected, and concentrated under reduced pressure. The obtained residue was suspended in EA (15 mL) and Hex (100 mL), then filtered. The filter cake was collected and dried to afford Compound 33-1 as a white solid (10.56 g, 93.6%). LCMS [M+1]=471.

To a solution of Compound 33-1 (2.04 g, 4.32 mmol) and INT B2 (1.54 g, 8.99 mmol) in DMSO (15 mL) was added KF (0.63 g, 10.84 mmol) at room temperature. The reaction mixture was stirred at 130° C. for 22 h. The reaction mixture was cooled to room temperature, diluted with EA (50 mL) and water (50 mL). The organic phase was collected and concentrated under reduced pressure. The residue was purified by silica gel chromatography (eluted with EA/Hex=1/4-1/2, v/v) to afford Compound 33-2 (1.57 g, 59.9%). LCMS [M+1]=606.

To a solution of Compound 33-2 (336 mg, 0.55 mmol) and INT C12 (387 mg, 0.78 mmol) in 1,4-dioxane (4 mL) and water (1 mL) were added Cs₂CO₃ (396 mg, 1.22 mmol) and Pd(dppf)Cl₂ (42 mg, 0.057 mmol). The reaction mixture was purged with nitrogen for three times and stirred at 100° C. for 2.3 h. The reaction mixture was cooled to room temperature, diluted with EA (20 mL) and water (20 mL). The organic phase was collected and concentrated under reduced pressure. The residue was purified by prep-TLC (eluted with EA/Hex=1/1, v/v) to afford Compound 33-3 (233 mg, 47.0%). LCMS [M+1]=894.

To a solution of Compound 33-3 (233 mg, 0.26 mmol) in 1,4-dioxane (4 mL) was added HCl/1,4-dioxane (4 M, 4 mL). The reaction mixture was stirred for 2 h at room temperature, then concentrated under reduced pressure. The obtained Compound 33-4 (crude) was used in next step directly without further purification. LCMS [M+1]=750.

To a solution of Compound 33-4 (crude) in DMF (2 mL) was added CsF (0.41 g, 2.70 mmol). The reaction mixture was stirred at room temperature for 2 h, then filtered and the filtrate was collected. CsF was mixed with the filtrate, and the mixture was stirred for another 18 h. The mixture was purified by Prep-HPLC (C18 column, 50*250 mm, 10 μm; A: 0.1% TFA in water, B:CH₃CN, Gradient: 15% B to 35% B in 30 min at a flow rate of 60 mL/min, 233 nm) to afford the TFA salt of Compound 33 (Compound 33-TFA, 86.8 mg, 40.5% for two steps) as a yellow solid. LCMS [M+1]=594.

### Example 3

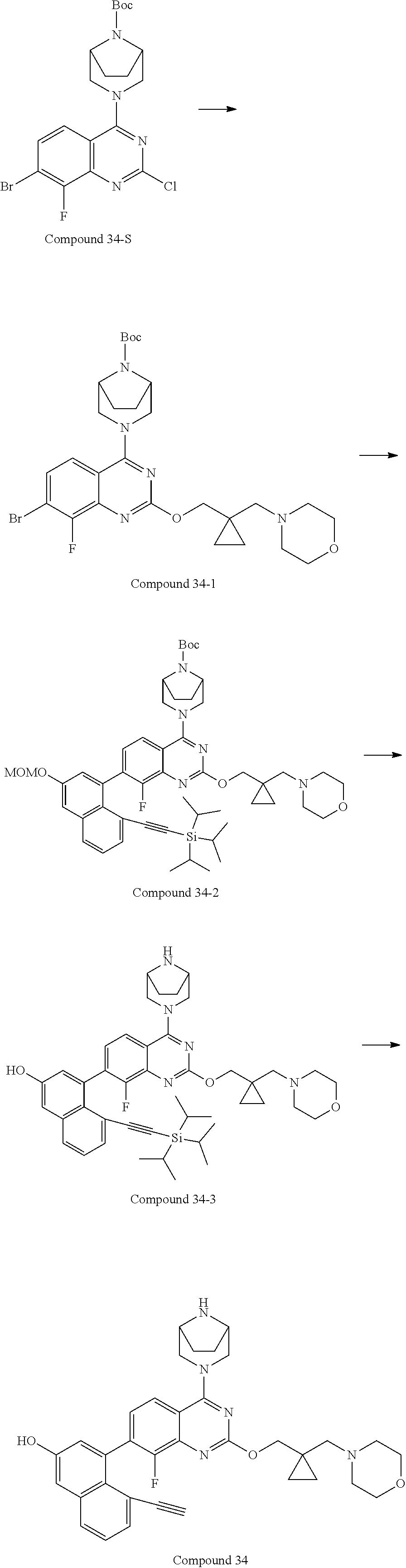

Compound 34-S was synthesized according to the similar procedures of preparing Compound 33-1.

A solution of Compound 34-S (518 mg, 1.10 mmol), INT B2 (375 mg, 2.19 mmol), KF (265 mg, 4.56 mmol) in DMSO (10 mL) was stirred at 130° C. for 20 h under nitrogen atmosphere. The mixture was allowed to cool to room temperature and partitioned between water and EA. The organic layer was washed with brine, dried over Na₂SO₄ and concentrated under vacuum. The residue was purified by Prep-TLC (n-hexane:EA=1:1, v/v) to afford the desired product Compound 34-1 (260 mg, 0.43 mmol). MS m/z: 606 [M+H]⁺.

To a solution of Compound 34-1 (259 mg, 0.43 mmol), INT C12 (256 mg, 0.52 mmol) in 1,4-dioxane (8 mL) and water (2 mL) was added Cs₂CO₃ (296 mg, 0.91 mmol) and Pd(dppf)Cl₂ (46 mg, 0.063 mmol). The reaction mixture was stirred at 100° C. for 2 hours under nitrogen atmosphere. The reaction was diluted with DCM (30 mL) and washed with water. The organic layer was dried over Na₂SO₄ and concentrated under vacuum. The residue was purified by Pre-TLC (n-hexane:EA=1:1, v/v) to afford Compound 34-2 (123 mg, 0.14 mmol). MS m/z: 894 [M+H]⁺.

A solution of Compound 34-2 (122 mg, 0.14 mmol), HCl/EA (4 M, 2.5 mL) in acetonitrile (5 mL) was stirred at RT for 1 h. The reaction was concentrated under vacuum to afford the crude product (Compound 34-3) which was used in next step without further purification (172 mg, 0.23 mmol). MS m/z: 750 [M+H]⁺.

A solution of Compound 34-3 (171 mg, 0.23 mmol), DIEA (10 drops) and CsF (525 mg, 3.46 mmol) in DMF (5 mL) was stirred at RT for 16 h, then 4 h at 45° C. The mixture was diluted with EA (30 mL) and washed with brine (3×30 mL). The organic layer was dried over Na₂SO₄ and concentrated under vacuum. The residue was purified by Prep-HPLC (Daisogel-C18 column, 50*250 mm, 10 μm; A: 0.1% TFA in water, B:CH₃CN, Gradient: 10% B to 35% B in 50 min at a flow rate of 60 mL/min, 220 nm) to afford the desired product (the TFA salt of Compound 34, Compound 34-TFA, 33.1 mg, 22.98%). MS m/z: 594 [M+H]⁺.

### Example 4

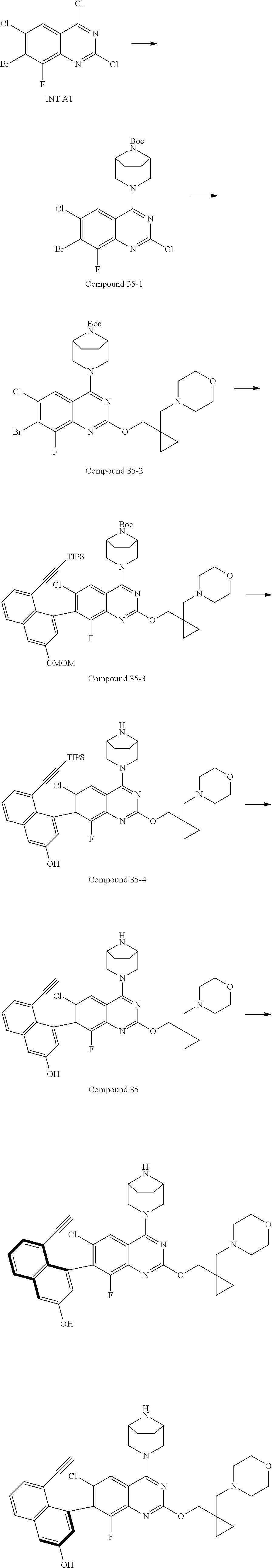

Tert-butyl (1R,5S)-3,8-diazabicyclo[3.2.1]octane-8-carboxylate (195 mg, 918.56 μmol) was added to a solution of INT A1 (305 mg, 923.21 μmol) and N, N-Diisopropylethylamine (249 mg, 1.93 mmol) dissolved in DCM (5.0 mL) at room temperature. The reaction mixture was poured into water (5 mL) after stirring for 1 h, and then extracted with DCM (10 mL×2). The organic layer was washed with water (10 mL×3) and brine (10 mL×2) successively, dried over Na₂SO₄ and then concentrated in vacuo to obtain Compound 35-1 (0.49 g, 968.00 μmol) as a yellow solid. LCMS: 505, 507 [M+H]⁺.

A mixture of Compound 35-1 (10.47 g, 20.68 mmol), INT B2 (5.28 g, 30.83 mmol), DABCO (1.13 g, 10.0738 mmol), and cesium carbonate (19.02 g, 58.38 mmol) dispersed in DMF (30 mL) and THF (30 mL) was stirred at room temperature for 16 hrs. The resulting mixture was diluted with EA (100 mL), washed with brine for three times. The organic layer was collected, dried over anhydrous Na₂SO₄, and then concentrated under reduced pressure to obtain a residue. The residue was purified by Pre-HPLC (eluted with 0-60% ACN in water (0.1% TFA)) to afford Compound 35-2 (7.07 g, 11.03 mmol, 53% yield). MS (ESI, m/z): 640 [M+1]⁺.

A mixture of Compound 35-2 (1018 mg, 1.59 mmol), INT C12 (1015 mg, 2.05 mmol), CataCXium A Pd G₃ (288 mg, 395.46 μmol), and Cs₂CO₃ (1483 mg, 4.55 mmol) dispersed in toluene (25 mL) and water (6 mL) was stirred at 100° C. for 5 hrs under nitrogen atmosphere. The resulting reaction was diluted with EA (100 mL) and washed with water (3×30 mL). The organic layer was dried over Na₂SO₄ and concentrated under vacuum to obtain a residue. The residue was purified by pre-HPLC (eluted with 0-70% ACN in water (0.1% TFA)) to afford Compound 35-3 (937 mg, 1.01 mmol, 63% yield). MS (ESI, m/z): 928 [M+1]⁺.

The Compound 35-3 (937 mg, 1.0090 mmol) was dissolved in CH₃CN (15 mL), and then HCl (4 M in 1,4-dioxane, 5 mL) was added. The reaction mixture was stirred at room temperature for 1 h, and then concentrated under reduced pressure to obtain a residue including Compound 35-4 which was used directly in next step without any purification. MS m/z: 784 [M+H]⁺.

The residue including Compound 35-4 was dissolved in DMF (20 mL), and then CsF (1.65 g, 10.86 mmol) was added. The reaction mixture was stirred at 40° C. for 16 hrs, and then concentrated under reduced pressure to obtain a residue. The residue was purified by Pre-HPLC (Daisogel-C18 column, 50×250 mm, 10 um; A: 0.1% TFA in water, B:CH₃CN, Gradient: 15% B to 40% B in 40 min at a flow rate of 200 mL/min, 250 nm) to afford the TFA salt of Compound 35 (0.32 g, 509.45 μmol, 50% yield). MS m/z: 628 [M+H]⁺.

The enantioseparation of the Compound 35 (37.5 mg) was performed by chiral-HPLC with the following condition: Equipment and Column: Prep CHIRAL ART Cellulose-SA column (2 cm×25 cm, 5 um) on Prep-HPLC-Gilson; Mobile phase: Hex (0.2% IPA·M)/EtOH (50:50); Flow Rate: 20 mL/min. This resulted in a first eluting stereoisomer (Compound 35A, 12.7 mg, Retention Time 4.107 min) and a second eluting stereoisomer (Compound 35B, 12.1 mg, Retention Time 6.851 min).

### Example 5

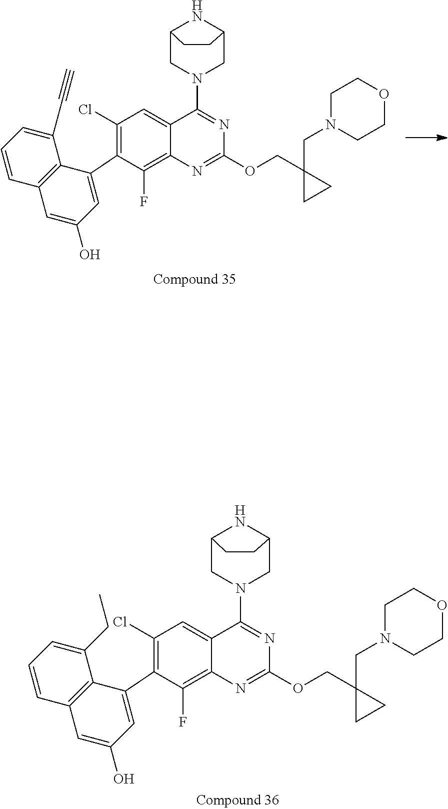 [SMILES: C#Cc1cccc2cc(O)cc(-c3c(Cl)cc4c(N5CC6CCC(C5)N6)nc(OCC5(CN6CCOCC6)CC5)nc4c3F)c12 ; CCc1cccc2cc(O)cc(-c3c(Cl)cc4c(N5CC6CCC(C5)N6)nc(OCC5(CN6CCOCC6)CC5)nc4c3F)c12]

Pd(OH)₂/C (15 mg, 106.81 μmol) was added to a solution of Compound 35 (44.8 mg, 71.32 μmol) dissolved in methanol (5 mL). The reaction mixture was stirred at room temperature for 2 hrs under hydrogen atmosphere, and then filtered. The filtrate was concentrated under reduced pressure and the residue was purified by pre-HPLC the following condition: Column, Venusil PrepG C18, 30*250 mm; Mobile Phase A, 0.1% TFA in water, Mobile Phase B, CH₃CN; Gradient: 15% B to 35% B in 35 min at a flow rate of 40 mL/min, 292 nm) to afford the TFA salt of Compound 36 (35.7 mg, 47.84 μmol, 67% yield, TFA salt). MS (ESI, m/z): 632 [M+1]⁺.

### Example 6

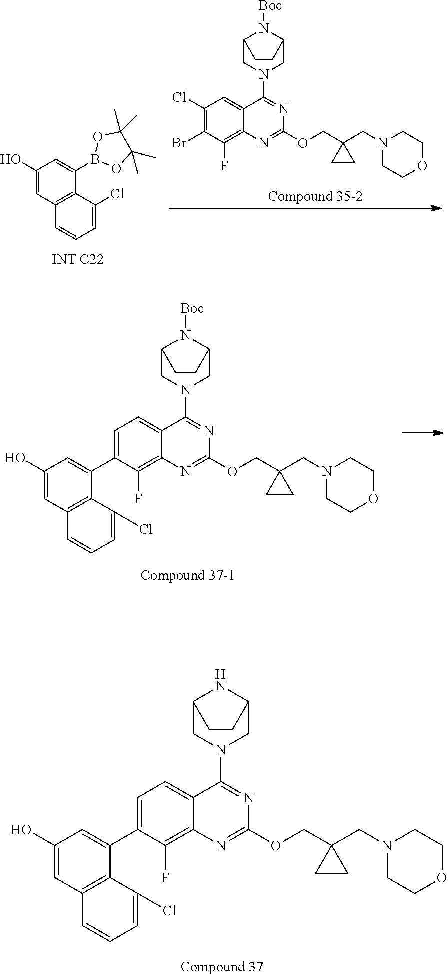

A mixture of Compound 35-2 (101 mg, 0.16 mmol), INT C22 (136 mg, 0.45 mmol), Pd(PPh₃)₄ (118 mg, 0.10 mmol), K₂CO₃ (186 mg, 1.35 mmol), toluene (2 mL), EtOH (1 mL) and water (0.5 mL) was stirred for 22 h at 100° C. under nitrogen atmosphere, cooled to room temperature and concentrated under vacuum to obtain a residue. The residue was purified by Prep-TLC, continued to purify by column chromatography eluted with Petroleum ether/Ethyl acetate (20:1, v/v), and then purified by prep-TLC eluted with MeOH:DCM (1:10) to afford Compound 37-1 (0.10 g, 0.14 mmol) as a brown solid. LCMS: 738 [M+H]⁺.

A mixture of Compound 37-1(0.10 g, 0.14 mmol), TFA (2 mL) and DCM (5 mL) was stirred for 2.5 hrs at room temperature, and then concentrated under vacuum to obtain a residue. The residue was purified by Prep-HPLC with the following conditions: column: Daisogel-C18 column, 50*250 mm; Mobile Phase A: 0.1% TFA in water, Mobile Phase B:CH₃CN; Gradient: 15% Mobile Phase B to 40% Mobile Phase B; Flow time: 40 mins; Flow rate of 60 mL/min; Detector Wavelength: 220 nm. This resulted in the TFA salt of Compound 37 (41.5 mg, 0.055 mmol). LCMS: m/z 638 [M+H]⁺.

### Example 7

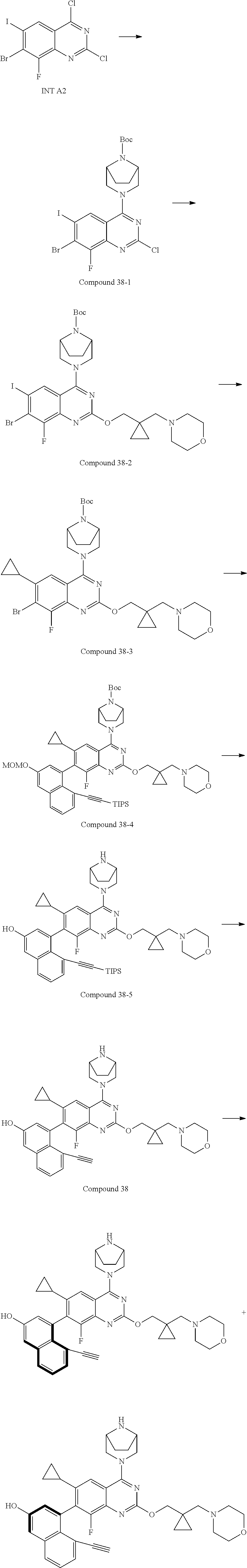

A mixture of INT A2 (18.24 g, 43.24 mmol), DIEA (10.61 g, 82.09 mmol) and tert-butyl (1R,5S)-3,8-diazabicyclo[3.2.1]octane-8-carboxylate (8.90 g, 41.92 mmol) and DCM (150 mL) was stirred at room temperature for 2 hrs. The resulting mixture was diluted with water (200 mL), and then extracted with DCM (200 mL). The combined organic layers were dried over Na₂SO₄ and concentrated in vacuum to obtain a residue. The residue was dispersed in Hex/EA (20:1, v/v, 240 ml) and then filtered. The filter cake was collected and dried to afford Compound 38-1 (20.48 g, 34.27 mmol). MS m/z: 597 [M+H]⁺.

A mixture of Compound 38-1 (3.06 g, 5.12 mmol), INT B2 (1.08 g, 6.31 mmol), DABCO (0.36 g, 3.21 mmol), Cs₂CO₃ (2.57 g, 7.89 mmol), DMF (12 mL) and THF (12 mL) was stirred at room temperature for 5 hrs, diluted with water (40 mL) and extracted with EA (2×40 mL). The organic layer was dried over Na₂SO₄ and concentrated in vacuum to obtain a residue. The residue was purified by Prep-HPLC with the following conditions: Column: C18 column; Mobile Phase A: 0.1% TFA in water; Mobile Phase B:CH₃CN; Gradient: 25% Mobile Phase B to 65% Mobile Phase B; Flow time: 40 min; Flow rate: 120 mL/min; Detector Wavelength: 254 nm. This resulted in Compound 38-2 (1.58 g, 2.16 mmol). MS m/z: 732 [M+H]⁺.

K₂CO₃ (354 mg, 2.56 mmol) and Pd(dppf)Cl₂ (85 mg, 0.11 mmol) were added to a solution of Compound 38-2 (750 mg, 1.02 mmol) and cyclopropylboronic acid (110 mg, 1.28 mmol) dissolved in toluene (10 mL) and water (1 mL). The reaction mixture was stirred at 100° C. for 7 hrs under nitrogen atmosphere, diluted with water (40 mL) and extracted with EA (2×30 mL). The combined organic layers were dried over Na₂SO₄ and concentrated under vacuum to obtain a residue. The residue was purified by Pre-TLC to afford Compound 38-3 (495 mg, 0.77 mmol). MS m/z: 646 [M+H]⁺.

Cs₂CO₃ (517 mg, 1.59 mmol) and cataCXium A Pd G₃ (70 mg, 0.096 mmol) were added to a solution of Compound 38-3 (495 mg, 0.77 mmol) and INT C12 (449 mg, 0.91 mmol) dissolved in toluene (10 mL) and water (2.5 mL). The reaction mixture was stirred at 100° C. for 20 hrs under nitrogen atmosphere, diluted with water (40 mL) and extracted with EA (2×30 mL). The combined organic layers were dried over Na₂SO₄ and concentrated under vacuum to obtain a residue. The residue was purified by Pre-TLC to obtain Compound 38-4 (424 mg, 0.72 mmol). MS m/z: 934 [M+H]⁺.

A solution of Compound 38-4 (424 mg, 0.72 mmol) and HCl (1.5 mL, 4 mol/L in dioxane) dissolved in CH₃CN (4.5 mL) was stirred at RT for 45 mins, and then concentrated in vacuum to obtain a residue containing Compound 38-5 which was directly used in next step without purification. MS m/z: 790 [M+H]⁺.

A mixture of the residue containing Compound 38-5, DIEA (0.5 mL), CsF (1.41 g, 9.28 mmol) and DMF (5 ml) was stirred at 45° C. for 2 hrs, filtered and CsF (1.46 g, 9.61 mmol) was added to the filtrate, continued to stir at 45° C. for 17 hrs. The resulting mixture was diluted with sat. aq. NaHCO₃ (40 mL) and extracted with EA (2×30 mL). The combined organic layers were dried over Na₂SO₄ and concentrated under vacuum to obtain a residue. The residue was purified by Pre-HPLC with the following conditions Column: Daisogel-C18; Mobile Phase A: 0.1% TFA in water; Mobile Phase B: CH₃CN; Gradient: 15% mobile Phase B to 35% mobile Phase B; Flow time: 35 mins; Flow rate: 60 mL/min; Detector Wavelength: 280 nm. This resulted in the TFA salt of Compound 38 (236.1 mg, TFA salt, 0.27 mmol). MS m/z: 634 [M+H]⁺.

The enantioseparation of the Compound 38 was performed by chiral-HPLC with the following condition: Equipment and Column: CHIRAL ART Cellulose-SA column (2 cm×25 cm, 5 um) on Prep-HPLC-Gilson; Mobile phase: Hex (0.1% IPA·M)/EtOH (50:50); Flow Rate: 20 mL/min. This resulted in a first eluting stereoisomer (Compound 38A, 79.6 mg, Retention Time 3.66 min) and a second eluting stereoisomer (Compound 38B, 65.8 mg, Retention Time 6.807 min).

### Example 8

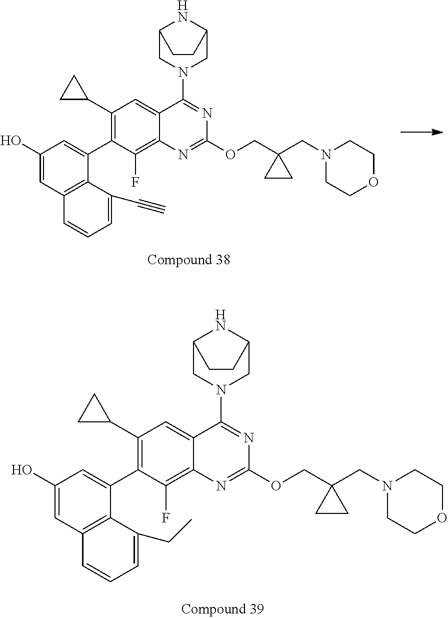 [SMILES: C#Cc1cccc2cc(O)cc(-c3c(C4CC4)cc4c(N5CC6CCC(C5)N6)nc(OCC5(CN6CCOCC6)CC5)nc4c3F)c12 ; CCc1cccc2cc(O)cc(-c3c(C4CC4)cc4c(N5CC6CCC(C5)N6)nc(OCC5(CN6CCOCC6)CC5)nc4c3F)c12]

A mixture of Compound 38 (9.8 mg, 0.015 mmol), Pd (OH)₂/C (15 mg, 20% Palladium content) and MeOH (6 mL) was stirred at room temperature for 1.5 hrs, filtered and the filtrate was concentrated under vacuum to afford a residue. The residue was dissolved in water and freeze dried under vacuum to obtain the TFA salt of Compound 39 (8.5 mg, 0.013 mmol). MS m/z: 638 [M+H]⁺.

### Example 9

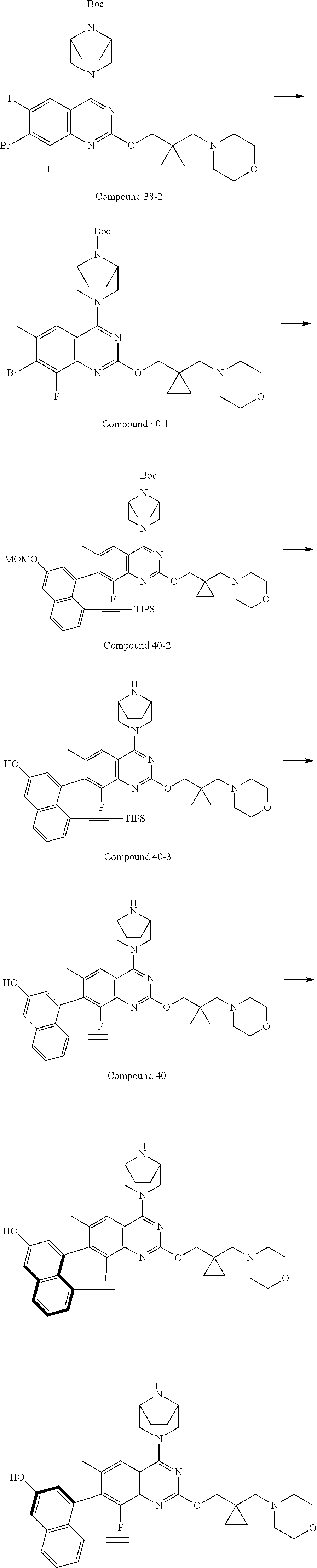

A mixture of Compound 38-2 (300 mg, 409.60 μmol), dipotassium carbonate (245 mg, 1.77 mmol), PdCl₂(dppf) (34 mg, 41.84 μmol), 2,4,6-trimethyl-1,3,5,2,4,6-trioxatriborinane (7 mL) and 1,4-dioxane (10 mL) was stirred at 70° C. for 36 hrs under nitrogen atmosphere, cooled to room temperature and diluted with EA (30 mL) and washed with water (30 mL). The organic layer was dried over Na₂SO₄ and concentrated under vacuum to afford a residue which was purified by Pre-TLC eluted with EA/HEX (1:2, v/v) to afford Compound 40-1 (0.3153 g, 508.10 μmol). MS m/z: 620 [M+H]⁺.

A mixture of Compound 40-1 (290 mg, 467.33 μmol), INT C12 (0.3029 g, 612.48 μmol), CataCXium A Pd G₃ (53.7 mg, 73.74 μmol), Cs₂CO₃ (0.4728 g, 1.45 mmol), toluene (10 mL) and water (2.5 mL) was stirred at 100° C. for 5 hours under nitrogen atmosphere, diluted with EA (30 mL) and then washed with water (30 mL). The organic layer was dried over Na₂SO₄ and concentrated under vacuum to obtain a residue which was purified by Pre-TLC eluted with MeOH/DCM (1:15, v/v) to afford Compound 40-2 (184 mg, 202.59 μmol, 43% yield). MS m/z: 908[M+H]⁺.

To a solution of Compound 40-2 (184 mg, 202.59 mol) in CH₃CN (10 mL) was added HCl (4 M in 1,4-dioxane, 2 mL). The reaction mixture was stirred at room temperature for 1 hour. The resulting mixture was concentrated under reduced pressure and the residue containing Compound 40-3 which was used directly to next step without purification. MS m/z: 764 [M+H]⁺.

To a solution of Compound 40-3 in DMF (5 mL) was added CsF (0.29 g, 1.91 mmol) and the reaction mixture was stirred at 40° C. for 16 hours. The resulting mixture was concentrated under reduced pressure and the residue was purified by pre-HPLC with the following conditions: Column: Venusil PrepG C18 column, 50×250 mm, 10 um; Mobile Phase A: 0.1% TFA in water; Mobile Phase B: CH₃CN; Gradient: 15% Mobile Phase B to 35% Mobile Phase B; Flow time: 40 mins; Flow rate: 40 mL/min; Detector Wavelength: 280 nm) to afford Compound 40 (32 mg, 44.34 μmol, 21% yield, TFA salt), MS m/z: 608 [M+H]⁺.

The enantioseparation of the Compound 40 (27.38 mg) was performed by chiral-HPLC with the following condition: Equipment and Column: CHIRAL ART Cellulose-SA column (2 cm×25 cm, 5 um) on Prep-HPLC-Gilson; Mobile phase: Hex (0.2% IPA·M)/EtOH (50:50); Flow Rate: 20 mL/min. This resulted in a first eluting stereoisomer (Compound 40A, 5.9 mg, Retention Time 3.943 min) and a second eluting stereoisomer (Compound 40B, 5.7 mg, Retention Time 7.861 min).

### Example 10

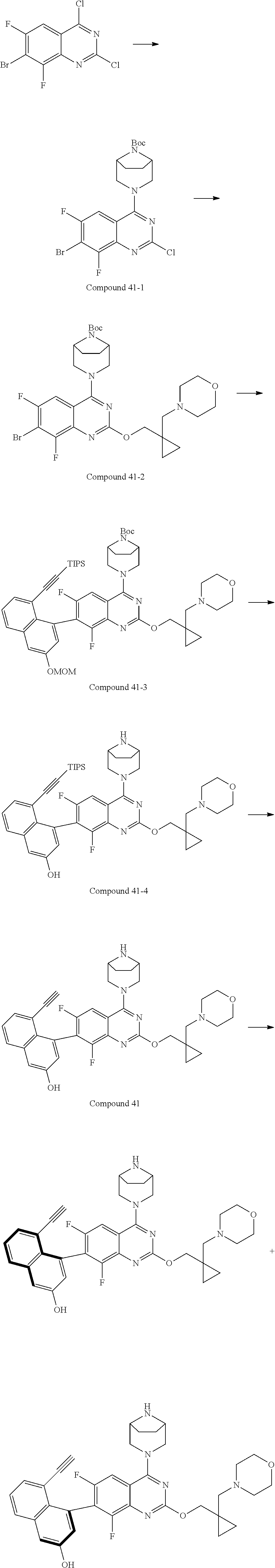

7-bromo-2,4-dichloro-6,8-difluoroquinazoline was synthesized with 3-bromo-2,4-difluorobenzenamine as starting material through using conventional preparation method.

DIEA (8.01 g, 61.98 mmol) was added to a solution of 7-bromo-2,4-dichloro-6,8-difluoroquinazoline (9.39 g, 29.91 mmol) and tert-butyl 3,8-diazabicyclo[3.2.1]octane-8-carboxylate (6.33 g, 29.82 mmol) dissolved in DCM (100 mL) at room temperature. The reaction mixture was stirred for 70 mins, and then water (50 mL) was added. The organic phase was collected, and concentrated under reduced pressure to obtain a residue. The residue was dispersed in EA (15 mL) and Hex (100 mL), and then filtered to afford Compound 41-1 as a white solid (15.13 g, 103.3%).

Triethylenediamine (0.13 g, 1.16 mmol) and Cs₂CO₃ were added to a solution of Compound 41-1 (0.97 g, 1.98 mmol) and INT B2 (0.56 g, 3.27 mmol) dissolved in THF (3 mL) and DMF (3 mL) at room temperature. The reaction mixture was stirred for 16 hrs at room temperature, then concentrated under reduced pressure to obtain a crude which was diluted with EA (20 mL) and water (20 mL). The organic phase was collected and concentrated under reduced pressure to obtain a residue. The residue was dispersed in EA (3 mL) and HEX (5 mL), and then filtered to afford Compound 41-2 (1.29 g, 104.29%). LCMS [M+1]=624.

Cs₂CO₃ (711 mg, 2.18 mmol) and cataCXium A Pd G₃ (122 mg, 0.17 mmol) were added to a solution of Compound 41-2 (631 mg, 1.01 mmol) and INT C12 (656 mg, 1.33 mmol) dissolved in toluene (4 mL) and water (1 mL). The reaction mixture was purged with N₂ for three times and stirred at 100° C. for 3 hrs, cooled to room temperature, diluted with EA (10 mL) and water (10 mL). The collected organic phases was concentrated under reduced pressure to obtain a residue which was purified by prep-TLC (EA/Hex=1/1, v/v) to afford Compound 41-3 (0.52 g, 56.4%). LCMS [M+1]=912.

HCl/dioxane (4 M, 4 mL) was added to a solution of Compound 41-3 (0.52 g, 0.57 mmol) dissolved in dioxane (4 mL). The reaction mixture was stirred for 18.5 hrs at room temperature, and concentrated under reduced pressure to obtain a residue. The residue was diluted with EA (10 mL) and water (10 mL), and which pH value was adjusted to 8 with KHCO₃. The organic phase was collected and concentrated under reduced pressure to afford a crude containing Compound 41-4 (crude) which was used directly in next step without further purification. LCMS [M+1]=768.

CsF (0.49 g, 3.23 mmol) was added to a solution of the crude containing Compound 41-4 dissolved in DMF (4 mL). The reaction mixture was stirred at room temperature for 24 hrs, and then filtered. The filtrate was concentrated under reduced pressure, and the residue was purified by Prep-HPLC with the following conditions: Column: Daisogel-C18 column, 50*250 mm, 10 μm; Mobile Phase A: 0.1% TFA in water; Mobile Phase B: CH₃CN; Gradient: 10% Mobile Phase B to 35% Mobile Phase B; Flow time: 40 mins; Flow rate: 40 mL/min; Detector Wavelength: 240 nm. This resulted in the TFA salt of Compound 41 as a yellow solid. LCMS [M+1]=612.

The enantioseparation of the Compound 41 was performed by chiral-HPLC with the following condition: Equipment and Column: CHIRAL ART Amylose-SA column (20*250 mm) on Prep-HPLC-Gilson; Mobile phase: Hex (0.1% IPA·M)/EtOH (50:50); Flow Rate: 20 mL/min. This resulted in a first eluting stereoisomer (Compound 41A, 44.7 mg, Retention Time 4.057 min) and a second stereoisomer (Compound 41B, 58.5 mg, Retention Time 6.767 min).

### Example 11

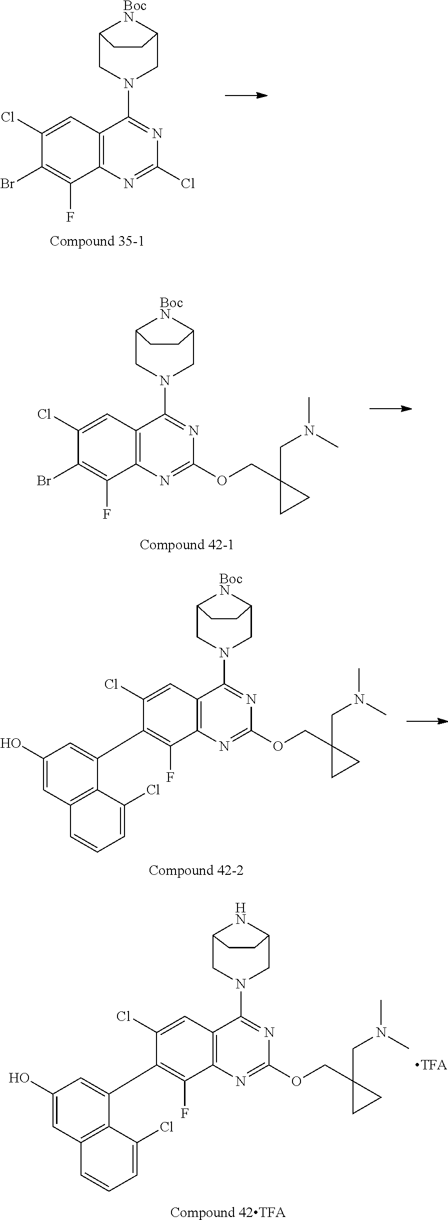

Compound 42-1 was synthesized following the procedure of Compound 35-2 with INT B1 and Compound 35-1 as starting material.

A mixture of Compound 42-1 (20 mg, 0.034 mmol, 1.0 eq), INT C22 (32 mg, 0.1 mmol, 3.0 eq), Na₂CO₃ (12 mg, 0.1 mmol, 3.0 eq), Pd(PPh₃)₄(4 mg, 0.0034 mmol, 0.1 eq) and dioxane/H₂O (4/1, 2 mL) was stirred at 100° C. for 6 hrs under N₂ atmosphere, cooled to room temperature, water (3 mL) was added, and then extracted with EtOAc (3 mL×3). The combined organic layers were washed with saturated brine, dried over anhydrous Na₂SO₄, and then concentrated to afford a residue containing Compound 42-2 which was used directly for next step without further purification. LCMS: 696.2[M+H]+.

TFA (0.3 mL) was added to a solution of Compound 42-2 (crude, 50 mg) dissolved in DCM (0.6 mL). The reaction mixture was stirred at 25° C. for 1 h, and concentrated to obtain a residue which was purified by pre-HPLC to afford the Compound 42-2TFA (13 mg) as a white solid. LCMS: 596.2 [M+H]⁺.

¹H NMR (300 MHz, DMSO-d₆): δ7.91 (s, 1H), 7.86 (dd, J=8.1, 1.3 Hz, 1H), 7.48-7.31 (m, 3H), 6.98 (d, J=2.5 Hz, 1H), 4.47-4.40 (m, 2H), 4.30-4.10 (m, 4H), 4.00-3.60 (m, 2H), 3.20 (d, J=5.6 Hz, 2H), 2.86 (s, 3H), 2.84 (s, 3H), 1.98-1.1.95 (m, 4H), 0.86-0.78 (m, 4H).

### Example 12

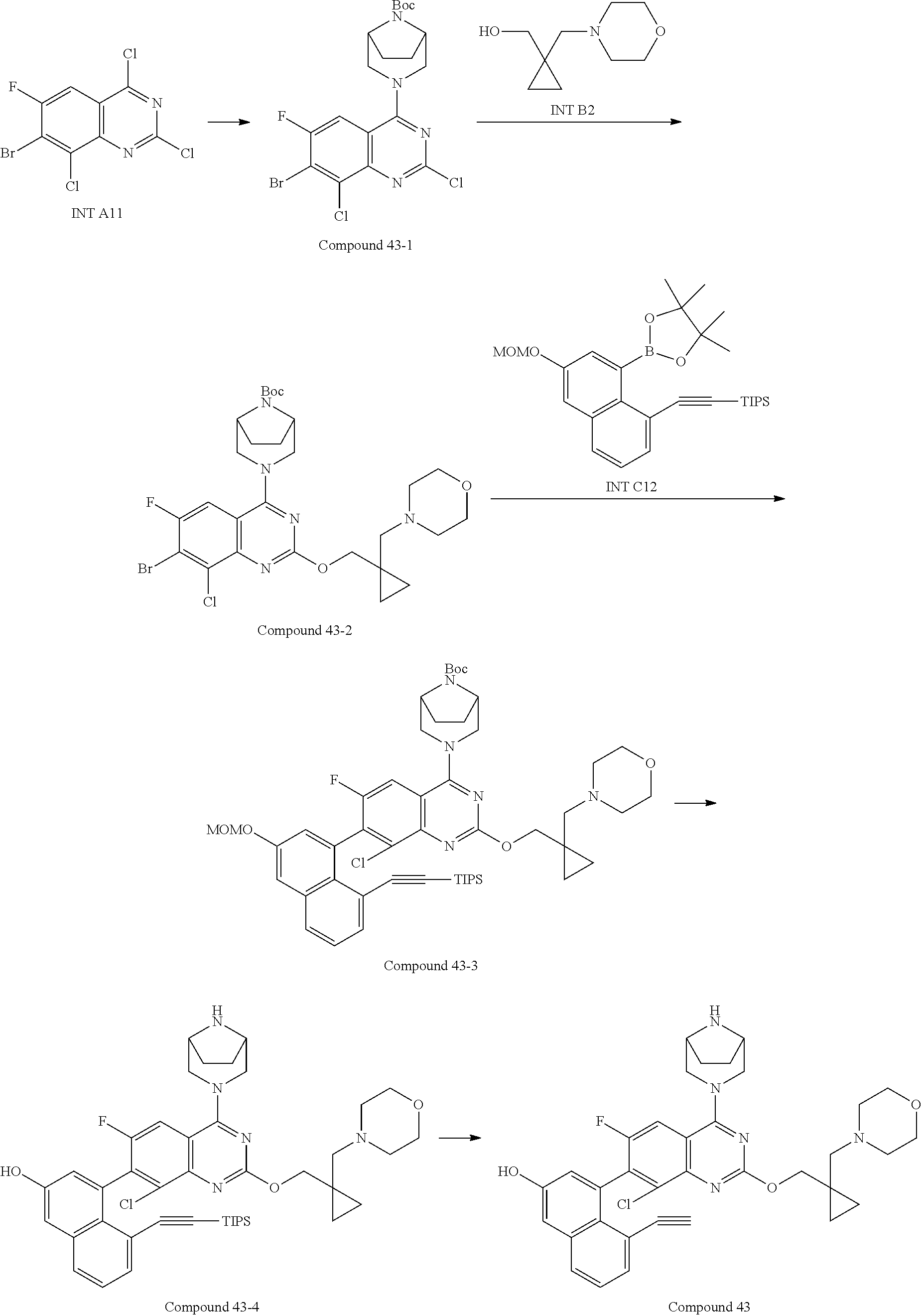

tert-butyl (1R,5S)-3,8-diazabicyclo[3.2.1]octane-8-carboxylate (510 mg, 2.4 mmol, 1.0 eq) and TEA (728 mg, 7.2 mmol, 3.0 eq) were added to a solution of INT A11 (2.2 g) in dioxane (8 mL). The reaction mixture was stirred at room temperature for 6 hrs, water (30 mL) was added, and then extracted with DCM (30 mL×3). The combined organic layers were washed with saturated aq. NH₄Cl (40 mL×2), dried over anhydrous Na₂SO₄, and concentrated to afford a residue which was purified by column chromatography (Petroleum ether/Ethyl acetate=5/1) to afford Compound 43-1 (850 mg, 1.7 mol, 70% yield) as a yellow solid. LCMS: 505.0 [M+H]⁺.

A mixture of Compound 43-1 (500 mg, 1.0 mmol, 1.0 eq), INT B2 (340 mg, 2.0 mmol, 2.0 eq), Cs₂CO₃ (980 mg, 3.0 mmol, 3.0 eq), DABCO (60 mg, 0.5 mmol, 0.5 eq) and THF/DMF (1/1, 10 mL) was stirred at room temperature for overnight, water (50 mL) was added, and extracted with EtOAc (50 mL×3). The combined organic layers were washed with brine and dried over anhydrous sodium sulfate, concentrated to afford a residue, purified by column chromatography (Petroleum ether/Ethyl acetate=1/1) to afford Compound 43-2 (150 mg, 0.23 mol, 23% yield) as a yellow solid LCMS: 640.2 [M+H]⁺. ¹H NMR (300 MHz, CDCl₃) δ 7.43 (d, J=9.3 Hz, 1H), 4.42 (s, 2H), 4.34 (s, 2H), 4.27-4.16 (m, 2H), 3.67-3.59 (m, 4H), 3.59-3.48 (m, 2H), 2.46 (s, 4H), 2.40 (s, 2H), 2.00-1.91 (m, 2H), 1.85-1.75 (m, 2H), 1.51 (s, 9H), 0.75-0.65 (m, 2H), 0.50-0.40 (m, 2H).

Compound 43-2 (100 mg, 0.16 mmol, 1.0 eq), INT C12 (225 mg, 0.47 mmol, 3.0 eq), Cs₂CO₃ (145 mg, 0.47 mmol, 3.0 eq) and Cata Cxium A Pd G3 (15 mg, 0.023 mmol, 0.15 eq) were placed in the reaction bottle. The bottle was purged with argon for three times, and then a solution of toluene/H₂O (4/1, 10 mL) was added at room temperature. The mixture was stirred at 100° C. for 4 hrs, water (30 mL) was added, and then extracted with EtOAc (3×30 mL). The combined organic layers were washed with saturated brine, dried over anhydrous Na₂SO₄, and then concentrated to afford a residue which was purified by pre-TLC to afford Compound 43-3 (60 mg, 0.065 mmol, 40% yield) as a light-yellow solid. ¹H NMR (300 MHz, DMSO-d₆): δ 8.03 (dd, J=9.6, 1.5 Hz, 1H), 7.69-7.61 (m, 2H), 7.59-7.56 (m, 1H), 7.53-7.51 (m, 1H), 7.15 (d, J=2.7 Hz, 1H), 5.37 (d, J=1.2 Hz, 2H), 4.59-4.54 (m, 1H), 4.34-4.27 (m, 4H), 3.86-3.82 (m, 1H), 3.57-3.54 (m, 4H), 3.45 (s, 3H), 2.41-2.37 (m, 6H), 2.28-2.26 (m, 2H), 2.03-1.85 (m, 4H), 1.46 (s, 9H), 0.86-0.79 (m, 21H), 0.66-0.63 (m, 2H), 0.48-0.43 (m, 2H). LCMS: 928.4 ([M+H]⁺).

HCl/dioxane (4 M, 0.65 ml) was added to a solution of Compound 43-3 (60 mg, 0.065 mmol, 1.0 eq) dissolved in ACN (3.2 mL) at 0° C. The reaction mixture was stirred at 0° C. for 3.5 hrs, and then concentrated to obtain a crude containing Compound 43-4 as a yellow solid which was used directly in the next step without further purification. LCMS: 784.3 [M+H]⁺.

CsF (198 mg, 1.3 mmol, 20.0 eq) was added to a solution of the crude containing Compound 43-4 in DMF (4.0 ml). The reaction mixture was stirred at 40° C. for overnight, and then purified by pre-HPLC to afford Compound 43 (16 mg, 0.026 mmol, 39% yield) as a white solid. LCMS: 628.2 [M+H]⁺.

¹H NMR (400 MHz, DMSO-d₆) δ 10.15 (s, 1H), 7.88 (dd, J=8.0, 1.6 Hz, 1H), 7.63 (d, J=10.1 Hz, 1H), 7.43 (dt, J=15.1, 6.3 Hz, 2H), 7.33 (d, J=2.5 Hz, 1H), 6.97 (d, J=2.5 Hz, 1H), 4.37-4.10 (m, 4H), 3.62-3.41 (m, 9H), 2.39 (s, 4H), 2.33-2.27 (m, 2H), 1.76-1.57 (m, 4H), 0.64 (s, 2H), 0.40 (s, 2H).

### Example 13

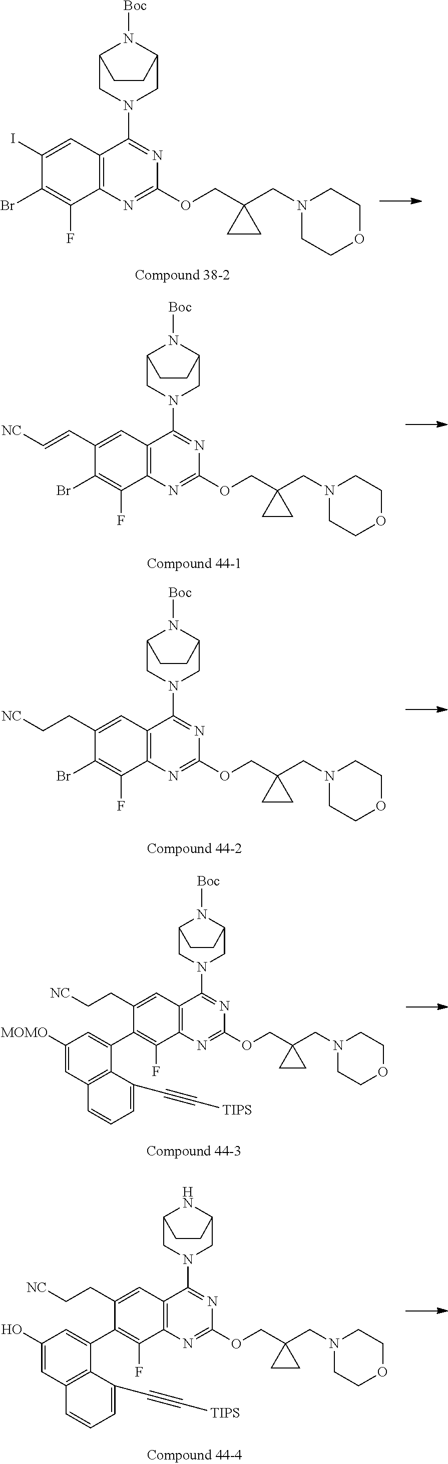

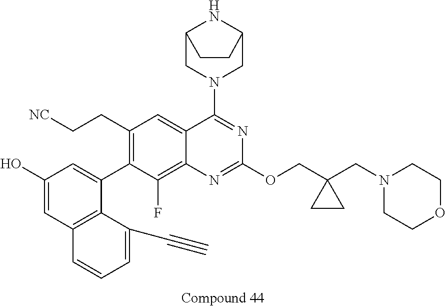 [SMILES: C#Cc1cccc2cc(O)cc(-c3c(CCC#N)cc4c(N5CC6CCC(C5)N6)nc(OCC5(CN6CCOCC6)CC5)nc4c3F)c12]

Pd(OAc)₂ (62 mg, 0.28 mmol), tri-o-tolylphosphane (110 mg, 0.36 mmol) and TEA (324 mg, 3.20 mmol) were added to a solution of Compound 38-2 (0.70 g, 0.96 mmol) and acrylonitrile (249 mg, 4.69 mmol) in DMF (10 mL). The reaction mixture was stirred at 90° C. for 6 hrs under nitrogen atmosphere, diluted with EA (40 mL) and washed with brine (40 mL). The organic layer was dried over Na₂SO₄ and then concentrated under reduced pressure to obtain a residue which was purified by Pre-TLC to afford Compound 44-1 (490 mg, 0.63 mmol). MS: m/z=657 [M+H]⁺.

LiBH₄ (1.2 mL, 2 mol/L in THF) was added to a solution of Compound 44-1 (299 mg, 0.45 mmol) in THF (10 mL). The reaction mixture was stirred at R.T for 3.5 hrs, quenched with water (30 mL), and then extracted with EA (40 mL). The organic layer was dried over Na₂SO₄ and then concentrated under reduced pressure to afford Compound 44-2 (265 mg, 0.30 mmol). MS m/z=659 [M+H]⁺.

Cs₂CO₃ (117 mg, 0.36 mmol) and cataCXium A Pd G₃ (31 mg, 0.043 mmol) were added to a solution of Compound 44-2 (101 mg, 0.15 mmol) and INT C12 (100 mg, 0.20 mmol) in toluene (4 mL) and water (1 mL). The reaction mixture was stirred at 100° C. for 17 hrs under nitrogen atmosphere, diluted with water (40 mL) and then extracted with EA (2×30 mL). The organic layers were combined, dried over Na₂SO₄ and then concentrated under reduced pressure to obtain a residue which was purified by Pre-TLC to afford Compound 44-3 (47 mg, 0.50 mmol). MS m/z=947 [M+H]⁺.

A mixture of Compound 44-3 (47 mg, 0.50 mmol), HCl (1 mL, 4 mol/L in 1,4-dioxane) and acetonitrile (3 mL) was stirred at R.T for 45 mins, and then concentrated under reduced pressure to obtain a residue. The residue was dispersed in saturated aq. NaHCO₃ (30 mL) and extracted with EA (2×30 mL). The organic layers were combined, dried over Na₂SO₄ and concentrated under reduced pressure to afford a crude containing Compound 44-4 (43 mg, 0.054 mmol). MS m/z=803 [M+H]⁺.

A mixture of the crude containing Compound 44-4 (43 mg, 0.054 mmol), CsF (0.40 g, 2.63 mmol) and DMF (4 mL) was stirred at 40° C. for 20 hrs, then filtered and the filtrate was concentrated under reduced pressure to obtain a residue which was purified by Pre-HPLC (C18 column; eluent A: 0.1% v/v TFA in water; eluent B: CH₃CN; Gradient: 15% B to 30% B in 35 mins at a flow rate of 40 mL/min; 242 nm) to afford the desired product (18.4 mg of the TFA salt of Compound 44, 0.021 mmol). MS m/z=647 [M+H]⁺.

### Example 14

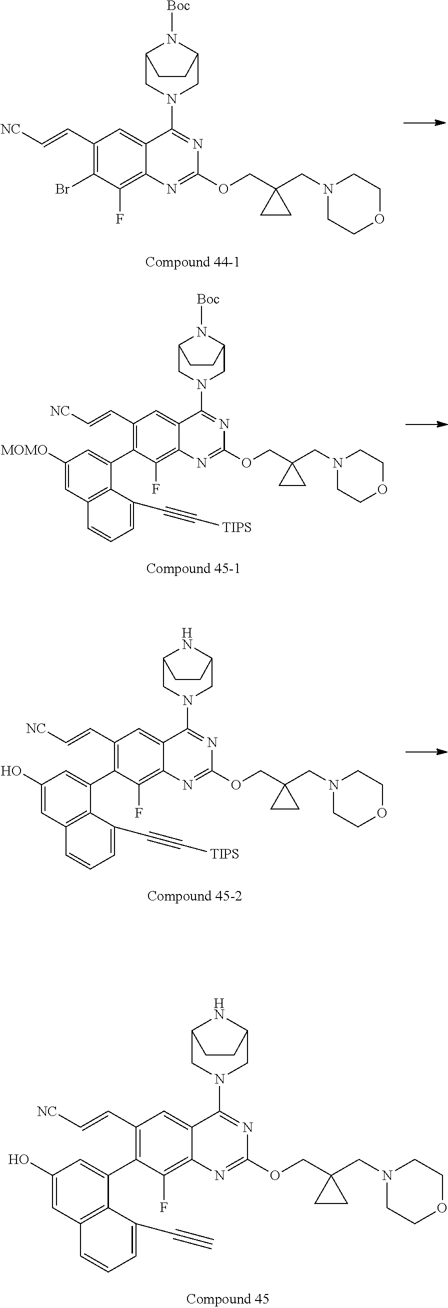

Cs₂CO₃ (107 mg, 0.33 mmol) and cataCXium A Pd G₃ (29 mg, 0.040 mmol) were added to a solution of Compound 44-1 (91 mg, 0.14 mmol), INT C12 (91 mg, 0.18 mmol) in toluene (4 mL) and water (1 mL). The reaction mixture was stirred at 100° C. for 22 hrs under nitrogen atmosphere, diluted with water (40 mL) and then extracted with EA (2×30 mL). The organic layers were combined, dried over Na₂SO₄ and then concentrated under reduced pressure to obtain a residue which was purified by Pre-TLC to afford Compound 45-1 (83 mg, 0.13 mmol). MS m/z=945 [M+H]⁺.

A solution of Compound 45-1 (83 mg, 0.13 mmol), HCl (1 mL, 4 mol/L in dioxane) in acetonitrile (3 mL) was stirred at R.T for 1 h, and then concentrated under reduced pressure to obtain a residue. The residue was diluted with saturated aq. NaHCO₃ (30 mL) and then extracted with EA (2×30 mL). The organic layers were combined, dried over Na₂SO₄ and then concentrated under reduced pressure to afford a crude containing Compound 45-2 (74 mg, 0.092 mmol). MS m/z=801 [M+H]⁺.

A mixture of the crude containing Compound 45-2 (74 mg, 0.092 mmol), CsF (0.78 g, 5.13 mmol) and DMF (4 mL) was stirred at 40° C. for 19 hrs, then filtered and the filtrate was concentrated under reduced pressure to obtain a residue which was purified by Pre-HPLC (C18 column; eluent A: 0.1% v/v TFA in water; eluent B: CH₃CN; Gradient: 15% B to 40% B in 35 min at a flow rate of 60 mL/min; 240 nm) to afford the desired product (46.2 mg of the TFA salt of Compound 45, 0.053 mmol). MS m/z=645 [M+H]⁺.

### Example 15

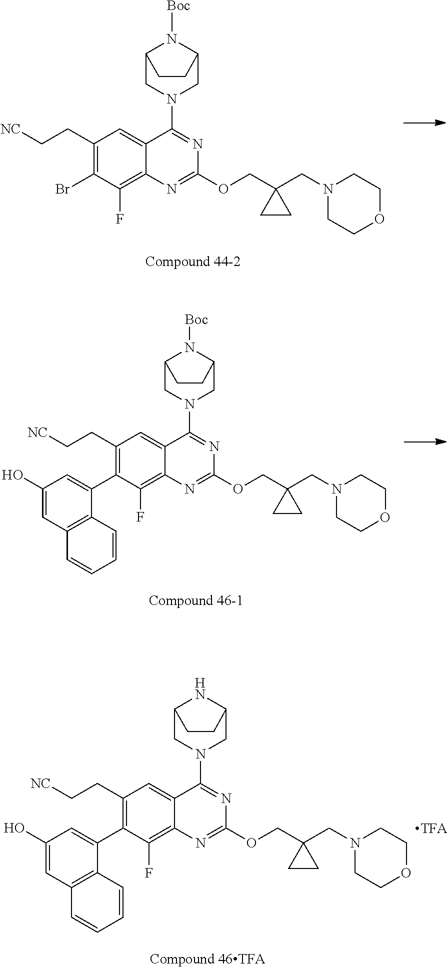 [SMILES: CC(C)(C)OC(=O)N1C2CCC1CN(c1nc(OCC3(CN4CCOCC4)CC3)nc3c(F)c(Br)c(CCC#N)cc13)C2 ; CC(C)(C)OC(=O)N1C2CCC1CN(c1nc(OCC3(CN4CCOCC4)CC3)nc3c(F)c(-c4cc(O)cc5ccccc45)c(CCC#N)cc13)C2 ; N#CCCc1cc2c(N3CC4CCC(C3)N4)nc(OCC3(CN4CCOCC4)CC3)nc2c(F)c1-c1cc(O)cc2ccccc12]

Na₂CO₃ (31 mg, 0.29 mmol) and Pd(PPh₃)₄(34 mg, 0.029 mmol) were added to a solution of Compound 44-2 (83 mg, 0.13 mmol) and INT C1 (62 mg, 0.23 mmol) in 1,4-dioxane (4 mL) and water (1 mL). The reaction mixture was stirred at 85° C. for 6 hrs under nitrogen atmosphere, diluted with water (40 mL) and then extracted with EA (2×30 mL). The organic layers were combined, dried over Na₂SO₄ and then concentrated under reduced pressure to obtain a residue which was purified by Pre-TLC to afford Compound 46-1 (54 mg, 0.075 mmol). MS m/z=723 [M+H]+.

A solution of Compound 46-1 (54 mg, 0.075 mmol) and TFA (1 mL) in DCM (4 mL) was stirred at R.T for 1 h, and then concentrated under reduced pressure to obtain a residue which was purified by Pre-HPLC (C18 column; eluent A: 0.1% v/v TFA in water; eluent B: CH₃CN; Gradient: 15% B to 35% B 5 in 30 min at a flow rate of 60 mL/min; 240 nm) to afford the desired product (23.3 mg of the TFA salt of Compound 46, 0.027 mmol). MS m/z=623 [M+H]⁺.

### Example 16

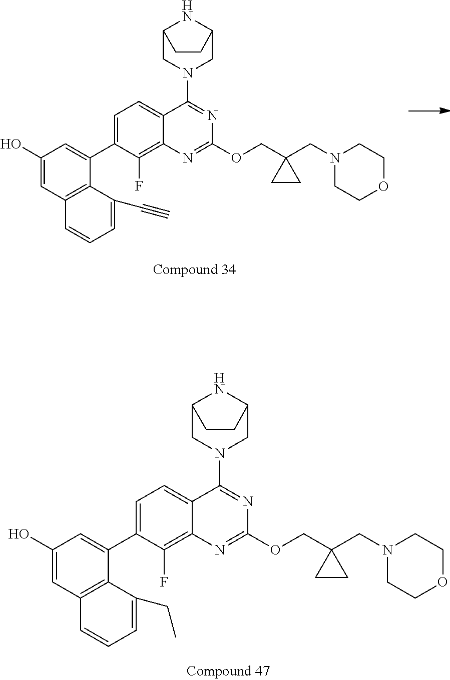 [SMILES: C#Cc1cccc2cc(O)cc(-c3ccc4c(N5CC6CCC(C5)N6)nc(OCC5(CN6CCOCC6)CC5)nc4c3F)c12 ; CCc1cccc2cc(O)cc(-c3ccc4c(N5CC6CCC(C5)N6)nc(OCC5(CN6CCOCC6)CC5)nc4c3F)c12]

A mixture of Compound 34 (67 mg, 0.11 mmol), Pd(OH)₂/C (43 mg, 20% palladium content) and 10 MeOH (10 mL) was stirred at R.T for 3 hrs under hydrogen atmosphere, then filtered and the filtrate was concentrated under reduced pressure to obtain a residue which was purified by Pre-HPLC (C18 column; eluent A: 0.1% v/v TFA in water; eluent B: CH₃CN; Gradient: 15% B to 40% B in 40 min at a flow rate of 60 mL/min; 280 nm) to afford Compound 47 (21.6 mg, 0.036 mmol). MS m/z=598 [M+H]⁺.

### Example 17

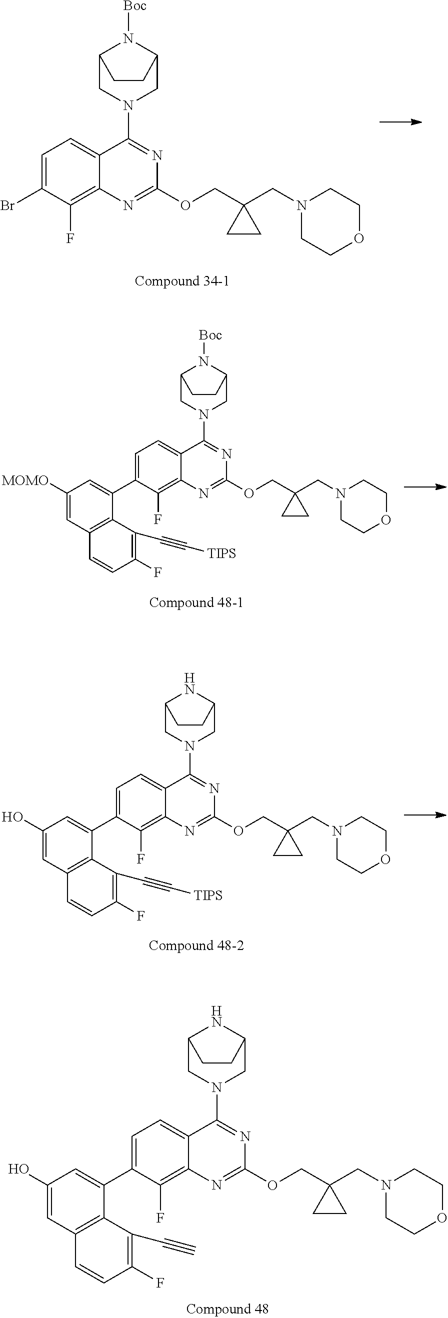

K₃PO₄ (674 mg, 3.18 mmol) and cataCXium A Pd G₃ (89 mg, 0.12 mmol) were added to a solution of Compound 34-1 (711 mg, 1.18 mmol) and INT C13 (723 mg, 1.41 mmol) in toluene (6 mL) and water (1.5 mL). The reaction mixture was stirred at 100° C. for 3 hrs under nitrogen atmosphere, diluted with water (40 mL) and then extracted with EA (40 mL). The collected organic layer was dried over Na₂SO₄ and then concentrated under reduced pressure to obtain a residue which was purified by Pre-TLC to afford Compound 48-1 (552 mg, 0.61 mmol). MS m/z=912 [M+H]⁺.

A mixture of Compound 48-1 (552 mg, 0.61 mmol), HCl (2 mL, 4 mol/L in 1,4-dioxane) and acetonitrile (6 mL) was stirred at R.T for 30 mins, and then concentrated under reduced pressure to obtain a residue containing Compound 48-2 which was directly used in the next step. MS m/z=768 [M+H]⁺.

A mixture of the residue containing Compound 48-2, DIEA (1 mL), CsF (1.83 g, 12.05 mmol) and DMF (5 mL) was stirred at 45° C. for 1 h, then filtered. A mixture of filtrate and CsF (1.80 g, 12.05 mmol) was stirred at 45° C. for 17 hrs, diluted with saturated aq. NaHCO₃ (40 mL) and then extracted with EA (2×30 mL). The organic layers were combined, dried over Na₂SO₄ and concentrated under reduced pressure to obtain a residue which was purified by Pre-HPLC (C18 column; eluent A: 0.1% v/v TFA in water; eluent B: CH₃CN; Gradient: 15% B to 35% B in 35 min at a flow rate of 60 mL/min; 285 nm) to afford Compound 48 (286 mg, 0.47 mmol). MS m/z=612 [M+H]⁺.

### Example 18

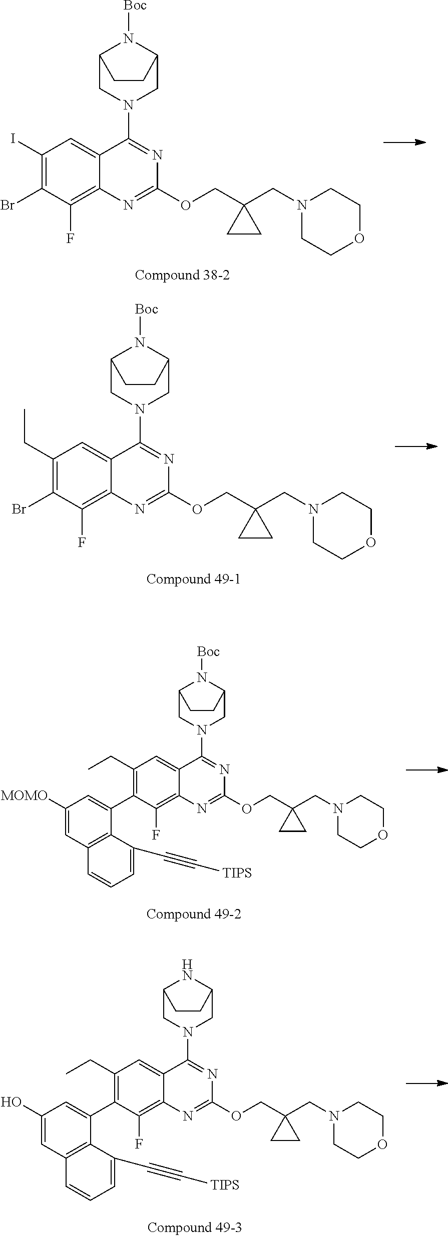

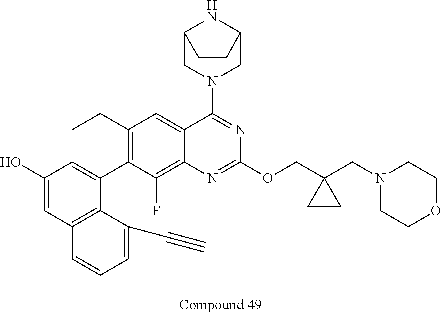 [SMILES: C#Cc1cccc2cc(O)cc(-c3c(CC)cc4c(N5CC6CCC(C5)N6)nc(OCC5(CN6CCOCC6)CC5)nc4c3F)c12]

Pd(dppf)Cl₂ (68 mg, 0.093 mmol) and K₂CO₃ (76 mg, 0.055 mmol) were added to a solution of Compound 38-2 (203 mg, 0.28 mmol) and ethylboronic acid (27 mg, 0.37 mmol) in toluylene (4 mL) and water (1 mL). The reaction mixture was stirred at 100° C. for 5 hrs under nitrogen atmosphere, diluted with EA (20 mL) and then washed with brine (40 mL). The organic layer were dried over Na₂SO₄ and then concentrated under reduced pressure to obtain a residue which was purified by Pre-TLC to afford Compound 49-1 (154 mg, 0.024 mmol). MS m/z=634 [M+H]⁺.

Cs₂CO₃ (147 mg, 0.45 mmol) and cataCXium A Pd G₃ (39 mg, 0.053 mmol) was added to a solution of Compound 49-1 (154 mg, 0.24 mmol) and INT C12 (135 mg, 0.27 mmol) in toluene (4 mL) and water (1 mL). The reaction mixture was stirred at 100° C. for 6 hrs under nitrogen atmosphere, diluted with water (20 mL) and extracted with EA (20 mL). The organic layers were combined, dried over Na₂SO₄ and concentrated under reduced pressure to obtain a residue which was purified by Pre-TLC to afford Compound 49-2 (151 mg, 0.16 mmol). MS m/z=922 [M+H]⁺.

A mixture of Compound 49-2 (151 mg, 0.16 mmol), HCl (1 mL, 4 mol/L in 1,4-dioxane) and acetonitrile (3 mL) was stirred at R.T for 1 h, and then concentrated under reduced pressure to obtain a residue. The residue was dispersed in saturated aq. NaHCO₃ (30 mL) and extracted with EA (2×20 mL). The organic layers were combined, dried over Na₂SO₄ and then concentrated under reduced pressure to afford a crude containing Compound 49-3 (130 mg, 0.17 mmol). MS m/z=778 [M+H]⁺.

A mixture of the crude containing Compound 49-3 (130 mg, 0.17 mmol), CsF (0.56 g, 3.69 mmol) and DMF (5 mL) was stirred at 40° C. for 22 hrs, then filtered and the filtrate was concentrated under reduced pressure to obtain a residue which was purified by Pre-HPLC (C18 column; eluent A: 0.1% v/v NH₃·H₂O in water; eluent B: CH₃CN; Gradient: 40% B to 70% B in 45 min at a flow rate of 70 mL/min; 236 nm) to afford Compound 49 (3.7 mg, 5.95 μmol). MS m/z=622 [M+H]⁺.

### Example 19

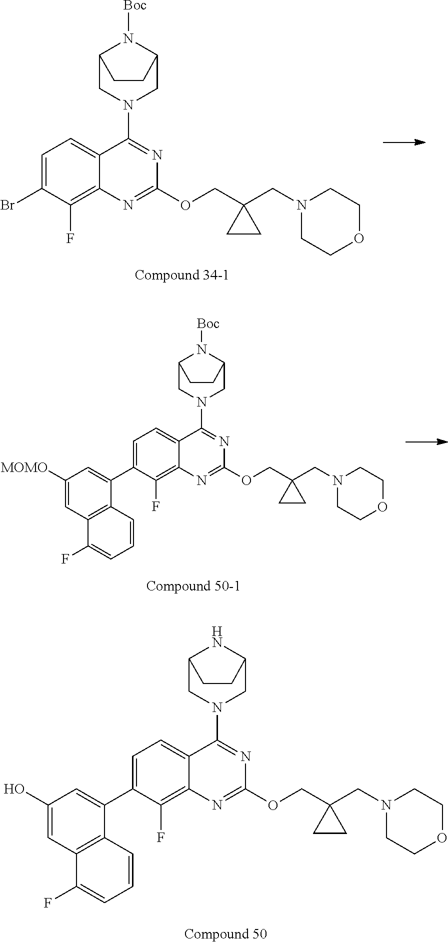

Na₂CO₃ (27 mg, 0.25 mmol) and Pd(PPh₃)₄(30 mg, 0.026 mmol) were added to a solution of Compound 34-1 (63 mg, 0.10 mmol) and INT C14 (48 mg, 0.14 mmol) in 1,4-dioxane (2 mL) and water (0.5 mL). The reaction mixture was stirred at 80° C. for 3.5 hrs under nitrogen atmosphere, diluted with water (20 mL) and then extracted with EA (20 mL). The organic layers were combined, dried over Na₂SO₄ and then concentrated under reduced pressure to obtain a residue which was purified by Pre-TLC to afford Compound 50-1 (80 mg, 0.11 mmol). MS m/z=732 [M+H]⁺.

A mixture of Compound 50-1 (80 mg, 0.11 mmol), HCl (1 mL, 4 mol/L in 1,4-dioxane) and acetonitrile (3 mL) was stirred at R.T for 1 h, and then concentrated under reduced pressure to obtain a residue which was purified by Pre-HPLC (C18 column; eluent A: 0.1% v/v TFA in water; eluent B: CH₃CN; Gradient: 15% B to 40% B in 35 min at a flow rate of 60 mL/min; 290 nm) to afford the desired product (51.9 mg of the TFA salt of Compound 50, 0.064 mmol). MS m/z=588 [M+H]⁺.

### Example 20

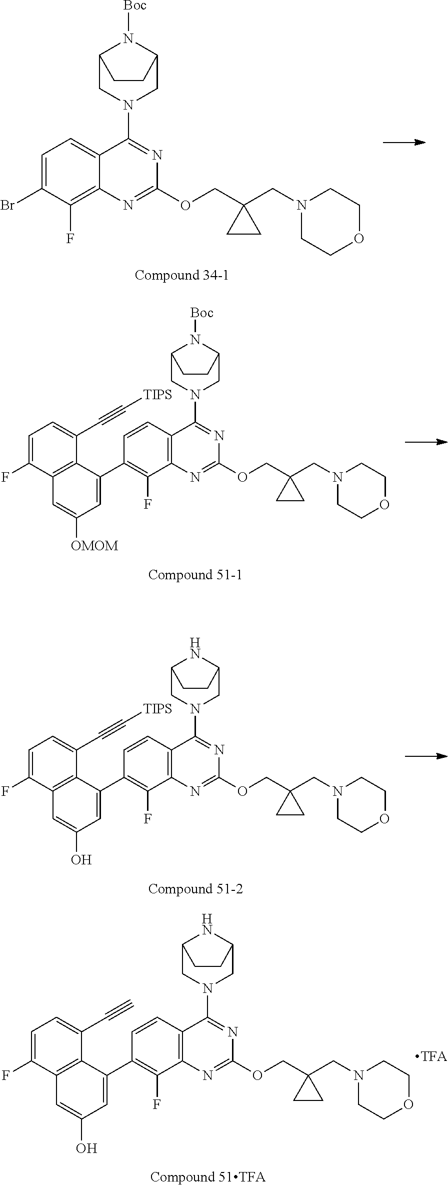

A mixture of Compound 34-1 (100 mg, 164.87 μmol), INT C15 (98 mg, 191.20 μmol), cataCXium A Pd G₃ (21 mg, 28.83 mol), K₃PO₄ (131 mg, 617.14 μmol), toluene (8 mL) and water (2.0 mL) was stirred at 100° C. for 2.5 hrs under nitrogen atmosphere, diluted with EA (50 mL) and washed with water (2×30 mL). The organic layer was dried over Na₂SO₄ and concentrated under reduced pressure to obtain a residue which was purified by Pre-TLC (MeOH:DCM=1:20, v/v) to afford Compound 51-1 (154 mg, 168.82 μmol, 102.3% yield). MS m/z=912 [M+H]⁺.

HCl (4 M in 1,4-dioxane, 2.0 mL) was added to a solution of Compound 51-1 (154 mg, 168.82 mol) in CH₃CN (6 mL). The reaction mixture was stirred at room temperature for 1 h, and then concentrated under reduced pressure to obtain a residue containing Compound 51-2 which was used directly in next step. MS m/z=768 [M+H]⁺.

CsF (0.29 g, 1.90 mmol) was added to a solution of the residue containing Compound 51-2 in DMF (5 mL). The reaction mixture was stirred at 40° C. for 16 hrs, concentrated under reduced pressure to obtain a residue which was purified by Pre-HPLC (Agela Venusil PrepG C18 column, 50*250 mm, 10 m; eluent A: 0.1% TFA in water; eluent B: CH₃CN; Gradient: 15% B to 45% B in 35 min at a flow rate of 60 mL/min; 240 nm) to afford the TFA salt of Compound 51 (102.9 mg, 141.79 μmol, 83.9% yield). MS m/z=612 [M+H]⁺.

### Example 21

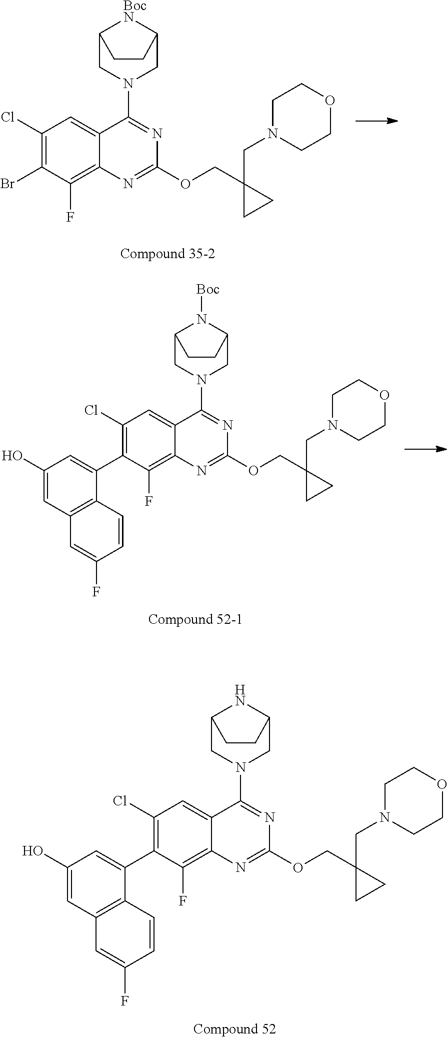 [SMILES: CC(C)(C)OC(=O)N1C2CCC1CN(c1nc(OCC3(CN4CCOCC4)CC3)nc3c(F)c(Br)c(Cl)cc13)C2 ; CC(C)(C)OC(=O)N1C2CCC1CN(c1nc(OCC3(CN4CCOCC4)CC3)nc3c(F)c(-c4cc(O)cc5cc(F)ccc45)c(Cl)cc13)C2 ; Oc1cc(-c2c(Cl)cc3c(N4CC5CCC(C4)N5)nc(OCC4(CN5CCOCC5)CC4)nc3c2F)c2ccc(F)cc2c1]

A mixture of Compound 35-2 (200.0 mg, 0.3 mmol, 1.3 eq), INT C16 (270.0 mg, 0.9 mmol, 3.0 eq), Pd(PPh₃)₄(72.0 mg, 0.06 mmol, 0.2 eq), Na₂CO₃ (100.0 mg, 0.9 mmol, 3 eq), dioxane (12 ml), and H₂O (3 ml) was stirred under N₂ atmosphere for 2 hrs at 100° C., and then concentrated to afford a residue which was purified by pre-HPLC to obtain Compound 52-1 (53.0 mg, 7.8% yield) as a white solid. LCMS m/z=722 ([M+H]⁺).

HCl/dioxane (4 M, 1.8 ml) was added to a solution of Compound 52-1 (53.0 mg, 0.08 mmol, 1.0 eq) in MeCN (9 mL). the reaction mixture was stirred at room temperature for 1.5 hrs, and then concentrated to obtain a crude which was purified by pre-HPLC to afford Compound 52 (23.0 mg, 50.0%) as a white solid. ¹H NMR (300 MHz, MeOD): δ 7.95 (d, J=1.8 Hz, 1H), 7.42-7.38 (m, 1H), 7.29-7.22 (m, 2H), 7.03-6.96 (m, 2H), 4.59-4.54 (m, 2H), 4.04 (s, 2H), 3.89 (s, 2H), 3.75-3.62 (m, 6H), 2.52-2.46 (m, 6H), 1.98-1.94 (m, 4H), 0.72-0.69 (t, J=4.2 Hz, 2H), 0.52-0.48 (t, J=4.5 Hz, 2H). LCMS: 622 ([M+H]⁺).

### Example 22

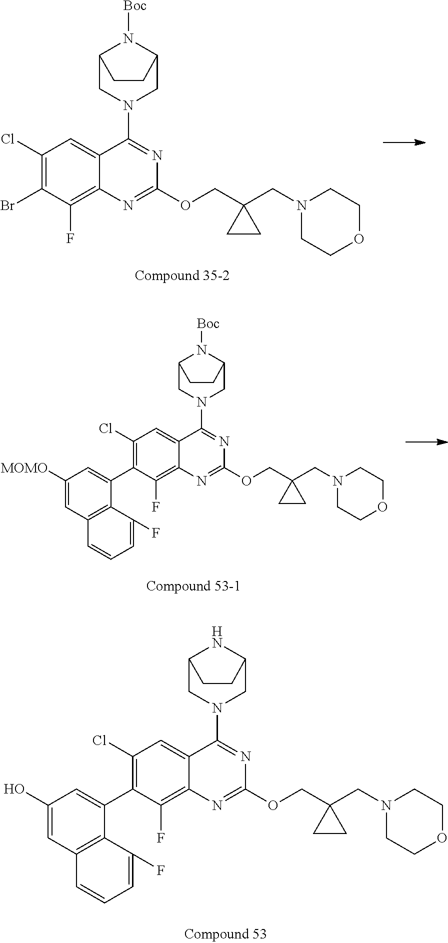

A mixture of Compound 35-2 (145.0 mg, 0.2 mmol, 1.0 eq), INT C17 (398.0 mg, 1.38 mmol, 6.0 eq), Na₂CO₃ (73.0 mg, 0.7 mmol, 3.0 eq), Pd (PPh₃)₄(23.0 mg, 0.02 mmol, 0.1 eq) were added successively into 1,4-dioxane (6.0 mL) and water (2.0 mL), then purged with argon for 10 mins, stirred overnight at 80° C. under argon atmosphere, cooled to room temperature, and then filtered. The filtrate was concentrated to obtain a residue which was purified by Pre-TLC (eluted with DCM/MeOH=10:1) to afford Compound 53-1 (40.0 mg, yield 23.1%) as a yellow solid. LCMS: 766 ([M+H]⁺).

A mixture of Compound 53-1 (40.0 mg, 0.05 mmol, 1.0 eq), MeCN (1 mL) and HCl/Dioxane (0.5 mL) was stirred at room temperature for 1 h, and then concentrated under reduced pressure to afford a crude product which was purified by Prep-HPLC. The eluent was lyophilizated to afford Compound 53 (5.6 mg, yield 17.5%) as a white solid. ¹H NMR (400 MHz, CD₃OD): δ 7.87-7.86 (m, 1H), 7.56 (d, J=8.4 Hz, 1H), 7.37-7.35 (m, 1H), 7.30-7.29 (m, 1H), 6.96 (d, J=2.4 Hz, 1H), 6.89-6.84 (m, 1H), 4.55-4.48 (m, 2H), 4.39 (s, 2H), 3.88-3.86 (m, 2H), 3.74-3.69 (m, 2H), 3.66-3.63 (m, 4H), 2.53-2.51 (m, 4H), 2.46 (s, 2H), 1.99-1.97 (m, 4H), 0.72-0.69 (m, 2H), 0.51-0.49 (m, 2H). LCMS: 622.1 ([M+H]⁺).

### Example 23

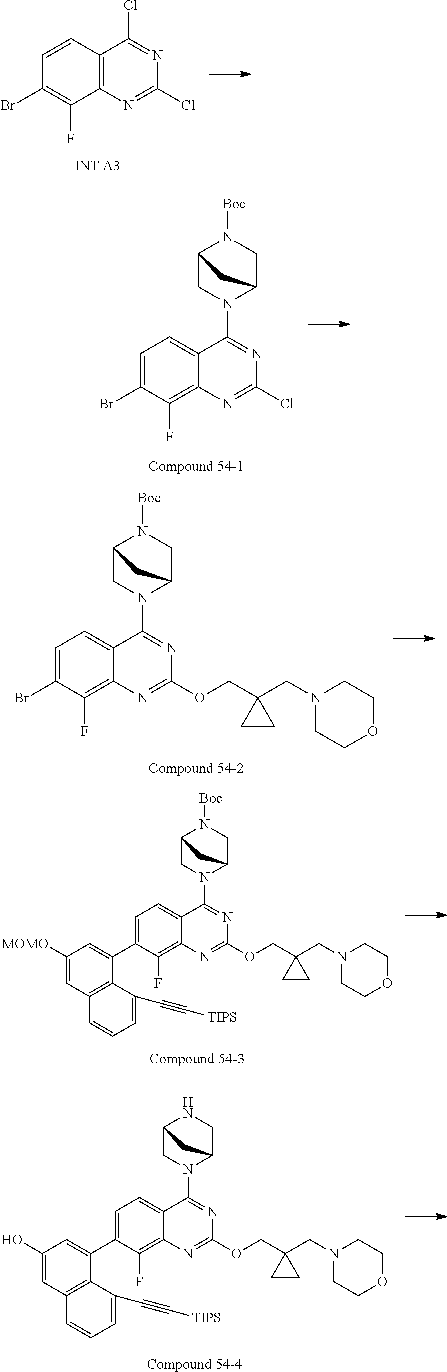

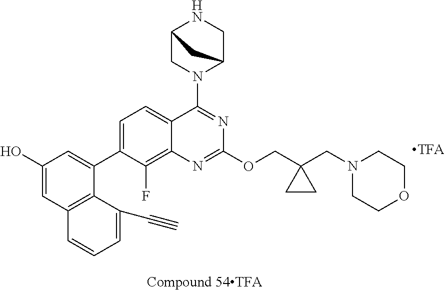 [SMILES: C#Cc1cccc2cc(O)cc(-c3ccc4c(N5C[C@H]6C[C@@H]5CN6)nc(OCC5(CN6CCOCC6)CC5)nc4c3F)c12 ; "Compound 54·TFA"]

A mixture of INT A3 (202 mg, 0.68 mmol), DIPEA (213 mg, 1.65 mmol), tert-butyl (1R,4R)-2,5-diazabicyclo[2.2.1] heptane-2-carboxylate (134 mg, 0.68 mmol) and DCM (10 mL) was stirred at room temperature for 1 h, and then concentrated under reduced pressure to obtain a residue. The residue was washed with a mixed solution of Hex and EA (V_{Hex}:V_{EA}=10:1, 20 mL) to afford Compound 54-1 (399 mg, 0.87 mmol). MS m/z: 457 [M+H]⁺.

A mixture of Compound 54-1 (399 mg, 0.87 mmol), INT B2 (196 mg, 1.14 mmol), DABCO (66 mg, 0.59 mmol), Cs₂CO₃ (592 mg, 1.82 mmol), DMF (4 mL) and THF (4 mL) was stirred at room temperature for 22 hrs, diluted with water (40 mL) and then extracted with EA (2×40 mL). The organic layers were combined, dried over Na₂SO₄ and concentrated under reduced pressure to obtain a residue which was washed with a mixed solution of Hex and EA (V_{Hex}:V_{EA}=10:1, 22 mL) to afford Compound 54-2 (312 mg, 0.53 mmol). MS m/z: 592 [M+H]⁺.

K₃PO₄ (107 mg, 0.50 mmol) and cataCXium A Pd G₃ (24 mg, 0.033 mmol) was added to a solution of Compound 54-2 (100 mg, 0.17 mmol) and INT C12 (101 mg, 0.20 mmol) in toluene (4 mL) and water (1 mL). The reaction mixture was stirred at 100° C. for 3.5 hrs under nitrogen atmosphere, diluted with water (20 mL) and then extracted with EA (20 mL). The organic layers were combined, dried over Na₂SO₄ and concentrated under reduced pressure to obtain a residue which was purified by Pre-TLC to afford Compound 54-3 (121 mg, 0.14 mmol). MS m/z: 880 [M+H]⁺.

A mixture of Compound 54-3 (121 mg, 0.14 mmol), HCl/1,4-dioxane (1 mL, 4 mol/L in 1,4-dioxane) and acetonitrile (3 mL) was stirred at R.T for 30 mins, and then concentrated under reduced pressure to obtain a residue. The residue was diluted with sat. aq. NaHCO₃ (30 mL) and extracted with EA (2×40 mL). The organic layers were combined, dried over Na₂SO₄ and concentrated under reduced pressure to afford Compound 54-4 (101 mg, 0.14 mmol). MS m/z: 736 [M+H]⁺.

A mixture of Compound 54-4 (101 mg, 0.14 mmol), CsF (0.63 g, 4.15 mmol) in DMF (5 mL) was stirred at 40° C. for 3.5 hrs, and then filtered. The filtrate was concentrated under reduced pressure to obtain a residue which was purified by Pre-HPLC (C18 column; eluent A: 0.1% v/v TFA in water; eluent B: CH₃CN; Gradient: 15% B to 35% B in 30 min at a flow rate of 60 mL/min; 245 nm) to afford the TFA salt of Compound 54 (49.7 mg, 0.11 mmol). MS m/z: 580 [M+H]⁺.

### Example 24

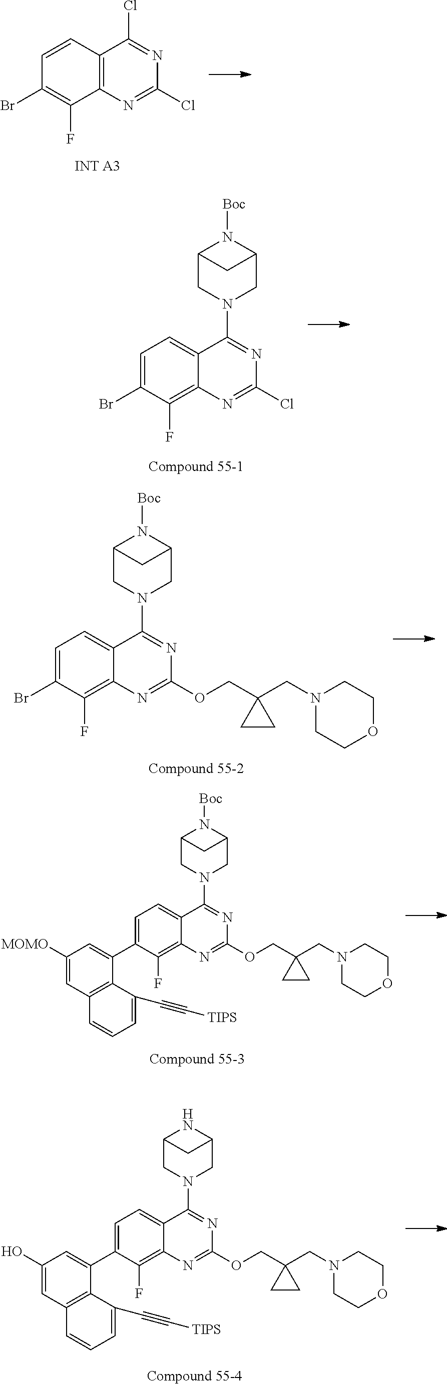

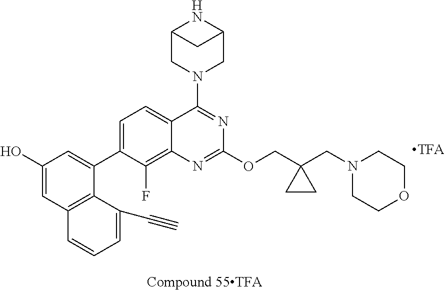 [SMILES: C#Cc1cccc2cc(O)cc(-c3ccc4c(N5CC6CC(C5)N6)nc(OCC5(CN6CCOCC6)CC5)nc4c3F)c12 ; "Compound 55·TFA"]

A mixture of INT A3 (109 mg, 0.37 mmol), DIPEA (114 mg, 0.88 mmol) and tert-butyl 3,6-diazabicyclo[3.1.1]heptane-6-carboxylate (78 mg, 0.39 mmol) and DCM (5 mL) was stirred at room temperature for 15 mins, and then concentrated under reduced pressure to obtain a residue. The residue was washed with a mixed solution of Hex and EA (V_{Hex}:V_{EA}=20:1, 20 mL) to afford Compound 55-1 (182 mg, 0.40 mmol). MS m/z: 457 [M+H]⁺.

A mixture of Compound 55-1 (182 mg, 0.40 mmol), INT B2 (113 mg, 0.66 mmol), DABCO (29 mg, 0.26 mmol), Cs₂CO₃ (202 mg, 0.62 mmol), DMF (3 mL) and THF (3 mL) was stirred at room temperature for 23 hrs, diluted with water (40 mL) and extracted with EA (2×40 mL). The organic layers were combined, dried over Na₂SO₄ and concentrated under reduced pressure to obtain a residue which was purified by Pre-TLC to afford Compound 55-2 (146 mg, 0.25 mmol). MS m/z: 592 [M+H]⁺.

K₃PO₄ (144 mg, 0.68 mmol) and cataCXium A Pd G₃ (31 mg, 0.043 mmol) was added to a solution of Compound 55-2 (146 mg, 0.25 mmol) and INT C12 (144 mg, 0.29 mmol) in toluene (4 mL) and water (1 mL). The reaction mixture was stirred at 100° C. for 2.5 hrs under nitrogen atmosphere, diluted with water (20 mL) and extracted with EA (20 mL). The organic layers were combined, dried over Na₂SO₄ and concentrated under reduced pressure to obtain a residue which was purified by Pre-TLC to afford Compound 55-3 (166 mg, 0.19 mmol). MS m/z: 880 [M+H]⁺.

A solution of Compound 55-3 (166 mg, 0.19 mmol) and HCl/1,4-dioxane (1 mL, 4 mol/L in 1,4-dioxane) in acetonitrile (3 mL) was stirred at R.T for 0.5 h, and then concentrated under reduced pressure to obtain a residue containing Compound 55-4 which was directly used in the next step. MS m/z: 736 [M+H]⁺.

A mixture of the residue containing Compound 55-4, DIEA (1 mL), CsF (0.62 g, 4.08 mmol) and DMF (3 mL) was stirred at 45° C. for 0.5 h, then filtered to obtain a filtrate. A mixture of the filtrate and CsF (0.72 g, 4.74 mmol) was stirred at 45° C. for 16 hrs, and then filtered to obtain the filtrate which was concentrated under reduced pressure to obtain a residue. The residue was purified by Pre-HPLC (C18 column; eluent A: 0.1% v/v TFA in water; eluent B: CH₃CN; Gradient: 15% B to 35% B in 30 min at a flow rate of 60 mL/min; 240 nm) to afford the TFA salt of Compound 55 (83.8 mg, 0.10 mmol). MS m/z: 580 [M+H]⁺.

### Example 25

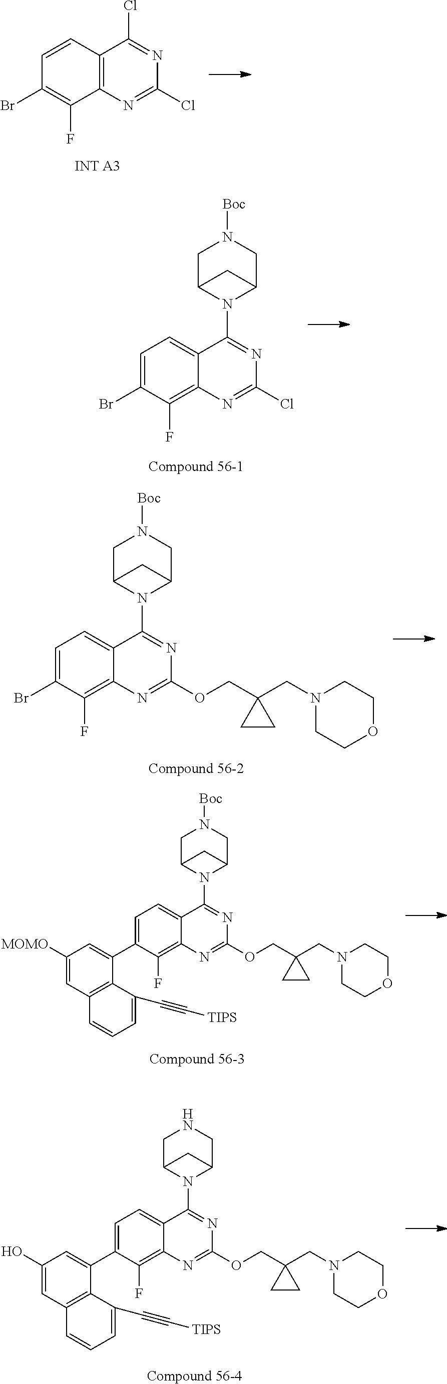

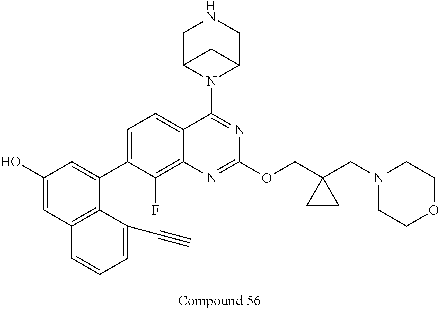 [SMILES: C#Cc1cccc2cc(O)cc(-c3ccc4c(N5C6CNCC5C6)nc(OCC5(CN6CCOCC6)CC5)nc4c3F)c12 ; "Compound 56"]

A solution of INT A3 (147 mg, 0.50 mmol), DIPEA (178 mg, 1.38 mmol) and tert-butyl 3,6-diazabicyclo[3.1.1]heptane-3-carboxylate (98 mg, 0.49 mmol) in DCM (10 mL) was stirred at room temperature for 0.5 h, and then concentrated under reduced pressure to obtain a residue. The residue was washed with a mixed solution of Hex and EA (V_{Hex}:VEA=20:1, 20 mL) to afford Compound 56-1 (284 mg, 0.62 mmol). MS m/z: 457 [M+H]⁺.

A mixture of Compound 56-1 (284 mg, 0.62 mmol), INT B2 (148 mg, 0.86 mmol), DABCO (43 mg, 0.38 mmol) and Cs₂CO₃ (308 mg, 0.95 mmol), DMF (4 mL) and THF (4 mL) was stirred at room temperature for 23 hrs, diluted with water (40 mL) and then extracted with EA (2×40 mL). The organic layers were combined, dried over Na₂SO₄ and concentrated under reduced pressure to obtain a residue which was purified by Pre-TLC to afford Compound 56-2 (231 mg, 0.39 mmol). MS m/z: 592 [M+H]⁺.

K₃PO₄ (112 mg, 0.53 mmol) and cataCXium A Pd G₃ (31 mg, 0.043 mmol) was added to a solution of Compound 56-2 (109 mg, 0.18 mmol) and INT C12 (108 mg, 0.22 mmol) in toluene (4 mL) and water (1 mL). The reaction mixture was stirred at 100° C. for 2.5 hrs under nitrogen atmosphere, diluted with water (20 mL) and then extracted with EA (20 mL). The organic layers were combined, dried over Na₂SO₄ and concentrated under reduced pressure to obtain a residue which was purified by Pre-TLC to afford Compound 56-3 (100 mg, 0.11 mmol). MS m/z: 880 [M+H]⁺.

A solution of Compound 56-3 (100 mg, 0.11 mmol) and HCl/1,4-dioxane (1 mL, 4 mol/L in 1,4-dioxane) in acetonitrile (3 mL) was stirred at R.T for 1 h, and then concentrated under reduced pressure to obtain a residue containing Compound 56-4 which was directly used in the next step. MS m/z: 736 [M+H]⁺.

A mixture of the residue containing Compound 56-4, DIEA (1 mL), CsF (0.61 g, 4.02 mmol) and DMF (5 mL) was stirred at 45° C. for 0.5 h, and then filtered to obtain a filtrate. A mixture of the filtrate and CsF (0.82 g, 5.40 mmol) was stirred at 45° C. for 5 hrs, and then filtered to obtain a filtrate which was concentrated under reduced pressure to obtain a residue. The residue was purified by Pre-HPLC (C18 column; eluent A: 0.1% v/v TFA in water; eluent B: CH₃CN; Gradient: 15% B to 35% B in 35 min at a flow rate of 60 mL/min; 240 nm) to afford the TFA salt of Compound 56 (35.8 mg, 0.044 mmol). MS m/z: 580 [M+H]⁺.

### Example 26

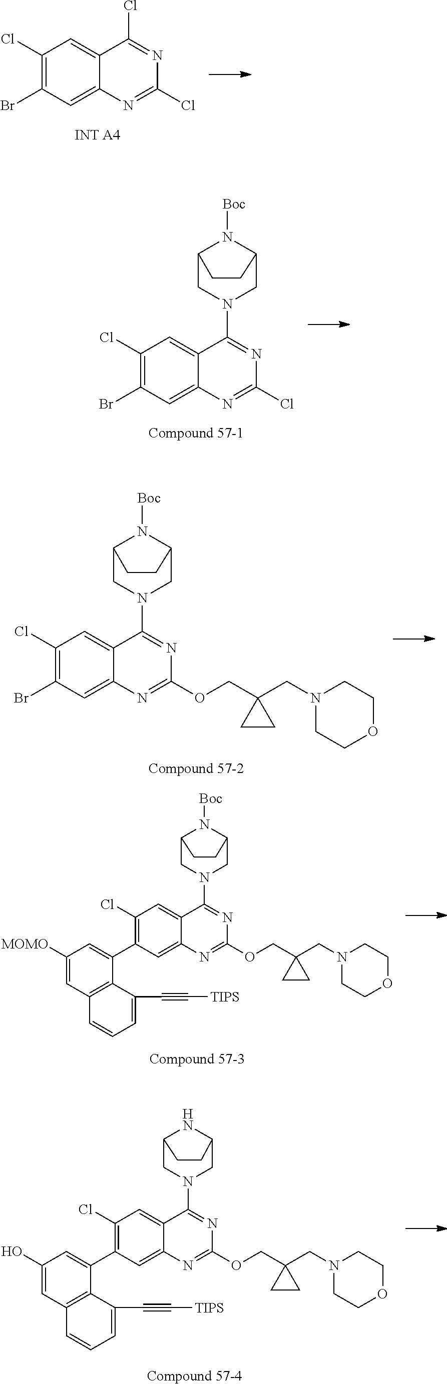

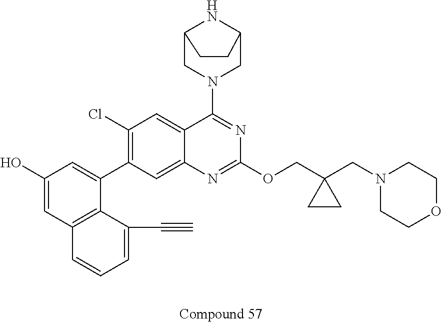 [SMILES: C#Cc1cccc2cc(O)cc(-c3cc4nc(OCC5(CN6CCOCC6)CC5)nc(N5CC6CCC(C5)N6)c4cc3Cl)c12 ; "Compound 57"]

A mixture of INT A4 (1.6 g, 5.12 mmol), DIPEA (1.29 g, 9.98 mmol) and tert-butyl (1R,5S)-3,8-diazabicyclo[3.2.1]octane-8-carboxylate (1 g, 4.71 mmol) and DCM (20 mL) was stirred at R.T for 1.5 hrs, diluted with water (40 mL) and extracted with EA (2×40 mL). The organic layers were combined, dried over Na₂SO₄ and concentrated under reduced pressure to obtain a residue. The residue was washed with a mixed solution of Hex and EA (V_{Hex}:V_{EA}=5:1, 30 mL) to afford Compound 57-1 (1.73 g, 3.54 mmol). MS m/z: 487 [M+H]⁺.

A mixture of Compound 57-1 (1.70 g, 3.48 mmol), INT B2 (1.04 g, 6.08 mmol), DABCO (220 mg, 1.96 mmol), Cs₂CO₃ (2.37 g, 7.28 mmol), DMF (8 mL) and THF (8 mL) was stirred at R.T for 15 hrs, and then concentrated under reduced pressure to obtain a residue. The residue was dispersed in water (20 mL) and extracted with EA (2×20 mL). The organic layers were combined, dried over Na₂SO₄ and concentrated under reduced pressure to obtain a residue. The residue was washed with water (20 mL) to afford Compound 57-2 (1.96 g, 3.15 mmol). MS m/z: 622 [M+H]⁺.

K₃PO₄ (193 mg, 0.91 mmol) and cataCXium A Pd G₃ (35 mg, 0.048 mmol) was added to a solution of Compound 57-2 (206 mg, 0.33 mmol) and INT C12 (189 mg, 0.38 mmol) in toluene (4 mL) and water (1 mL). The reaction mixture was stirred at 100° C. for 3 hours under nitrogen atmosphere, diluted with water (20 mL) and extracted with EA (20 mL). The organic layers were combined, dried over Na₂SO₄ and concentrated under reduced pressure to obtain a residue which was purified with Pre-TLC to afford Compound 57-3 (166 mg, 0.18 mmol). MS m/z: 910 [M+H]⁺.

A solution of Compound 57-3 (166 mg, 0.18 mmol) and HCl (2 mL, 4 mol/L in 1,4-dioxane) dissolve in acetonitrile (6 mL) was stirred at R.T for 1 h, and then concentrated under reduced pressure to obtain a residue containing Compound 57-4 which was directly used in the next step. MS m/z: 766 [M+H]⁺.

A mixture of the residue containing Compound 57-4, DIEA (1 mL), CsF (0.88 g, 5.79 mmol) and DMF (6 mL) was stirred for 2 hrs at 45° C., and then filtered. A mixture of the filtrate and CsF (0.94 g, 6.18 mmol) was stirred at 45° C. for 16 hrs, and then filtered to obtain a filtrate which was concentrated under reduced pressure to afford a residue. The residue was purified with Pre-HPLC (C18 column; eluent A: 0.1% v/v TFA in water; eluent B: CH₃CN; Gradient: 15% B to 35% B in 35 min at a flow rate of 60 mL/min; 285 nm) to afford Compound 57 (79.3 mg TFA salt, 0.095 mmol). MS m/z: 610 [M+H]⁺.

### Example 27

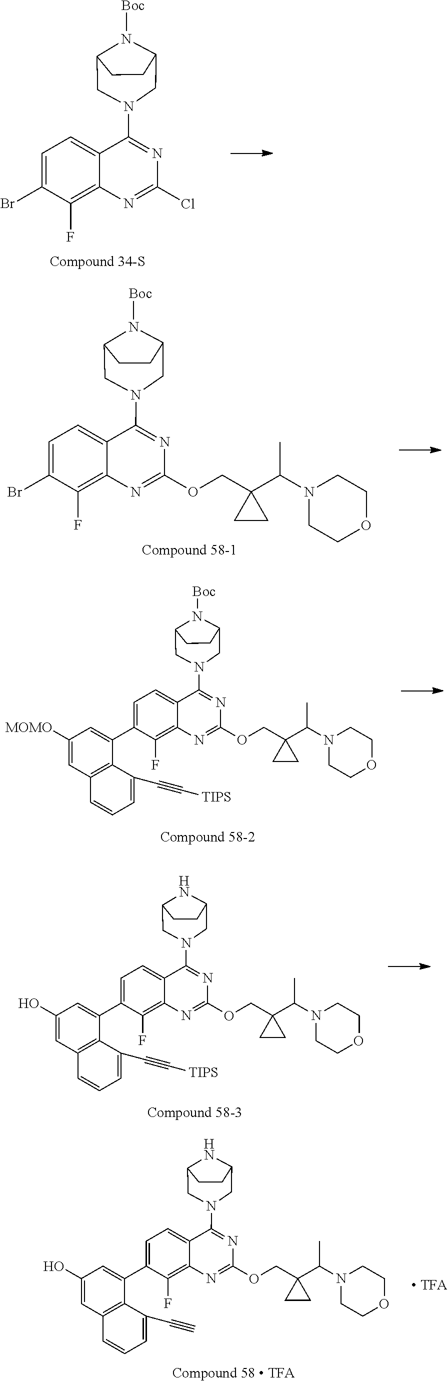

A mixture of Compound 34-S (306 mg, 0.65 mmol), INT B16 (123 mg, 0.66 mmol), KF (228 mg, 3.92 mmol) and DMSO (8 mL) was stirred at 120° C. for 22 hrs, diluted with water (40 mL) and extracted with EA (2×40 mL). The organic layers were combined, dried over Na₂SO₄ and concentrated under reduced pressure to obtain a residue which was purified with Pre-TLC to afford Compound 58-1 (116 mg, 0.40 mmol). MS m/z: 620 [M+H]⁺.

Na₂CO₃ (71 mg, 0.67 mmol) and Pd(dppf)Cl₂ (51 mg, 0.070 mmol) was added to a solution of Compound 58-1 (109 mg, 0.18 mmol) and INT C12 (121 mg, 0.24 mmol) in 1,4-dioxane (4 mL) and water (1 mL). The reaction mixture was stirred at 85° C. for 2 hrs under nitrogen atmosphere, diluted with water (20 mL) and extracted with EA (20 mL). The organic layers were combined, dried over Na₂SO₄ and concentrated under reduced pressure to obtain a residue which purified with Pre-TLC to afford Compound 58-2 (55 mg, 0.061 mmol). MS m/z: 908 [M+H]⁺.

A solution of Compound 58-2 (55 mg, 0.061 mmol) and HCl (2.5 mL, 4 mol/L in 1,4-dioxane) in acetonitrile (5 mL) was stirred at R.T for 1.5 hrs, and then concentrated under reduced pressure to obtain a residue containing Compound 58-3 which was used directly in the next step. MS m/z: 764 [M+H]⁺.

A mixture of the residue containing Compound 58-3, DIEA (0.5 mL), CsF (0.88 g, 5.79 mmol) and DMF (5 mL) was stirred at 40° C. for 16 hrs, and then filtered. The filtrate was concentrated under reduced pressure to obtain a residue which was purified with Pre-HPLC (C18 column; eluent A: 0.1% v/v TFA in water; eluent B: CH₃CN; Gradient: 10% B to 45% B in 40 min at a flow rate of 60 mL/min; 230 nm) to afford the TFA salt of Compound 58 (15.1 mg, 0.018 mmol). MS m/z: 608 [M+H]⁺.

### Example 28

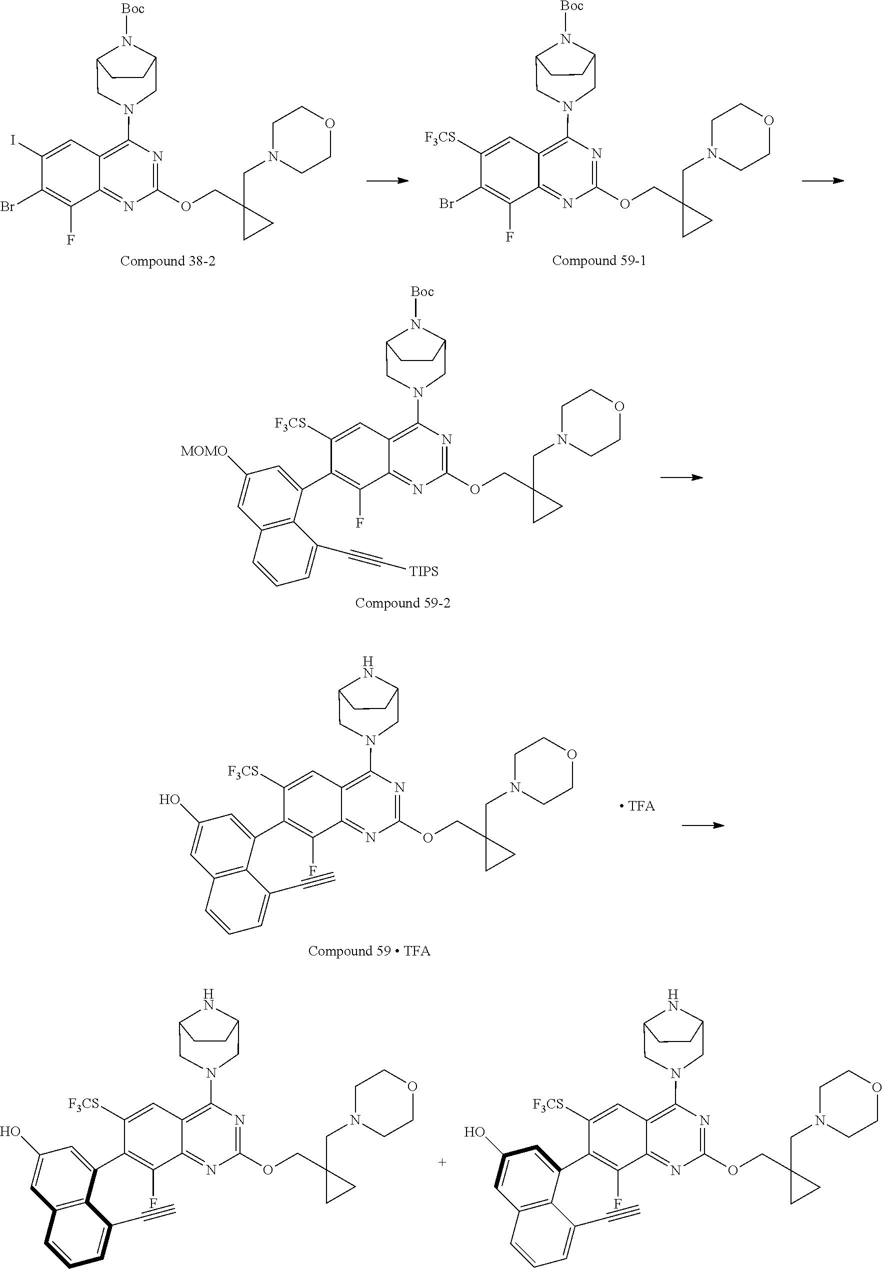

A mixture of Compound 38-2 (305 mg, 0.416 mmol), (trifluoromethylthio) silver (I) (304 mg, 1.46 mmol), CuI (800 mg, 4.20 mmol) and DMF (2.5 mL) was stirred at 95° C. for 3 hrs under nitrogen atmosphere, and then filtered. The filtrate was concentrated under reduced pressure to obtain a residue which was purified with Pre-TLC (Hex:EA=3:1, v/v) to afford Compound 59-1. MS m/z: 706 [M+H]⁺.

Cs₂CO₃ (74 mg, 0.227 mmol) and cataCXium A Pd G₃ (8 mg, 0.011 mmol) was added to a solution of Compound 59-1 (71 mg, 0.10 mmol), INT C12 (59 mg, 0.12 mmol) in toluene (4 mL) and water (1 mL). The reaction mixture was stirred overnight at 100° C. under nitrogen atmosphere, diluted with water (20 mL) and extracted with EA (2×20 mL). The organic layers were combined, dried over Na₂SO₄ and concentrated under reduced pressure to obtain a residue which was purified with Pre-TLC (Hex:EA=3:1, v/v) to afford Compound 59-2 (63 mg, 0.063 mmol). MS m/z: 994 [M+H]⁺.

A solution of Compound 59-2 (63 mg, 0.063 mmol) and HCl (1 mL, 1 M in 1,4-dioxane) in CH₃CN (3 mL) was stirred at R.T for 1 h, and then concentrated under reduced pressure to obtain a residue. The residue was diluted with saturated aq. NaHCO₃ (20 mL) and extracted with EA (2×20 mL). The organic layers were combined, dried over Na₂SO₄ and concentrated under reduced pressure to obtain a residue. The mixture of the residue, CsF (0.12 g, 0.79 mmol) and DMF (3 mL) was stirred at 40° C. for 2 hours under nitrogen atmosphere. The mixture was diluted with EA (20 mL), and washed with sat. NaHCO₃ (20 mL) and brine (20 mL). The collected organic layer was dried over Na₂SO₄, concentrated, and then purified with Prep-HPLC (C18 column; eluent A: 0.1% TFA in water; eluent B: CH₃CN; Gradient: 10% B to 40% B in 40 min at a flow rate of 40 mL/min; 242 nm) to afford the TFA salt of Compound 59 (28.8 mg). MS m/z: 694 [M+H]⁺.

The enantioseparation of the Compound 59 was performed by chiral-HPLC with the following condition: CHIRAL ART Amylose-SA column (2 cm×25 cm, 5 um) on Prep-HPLC-Gilson; Mobile phase: Hex (0.1% IPA·M)/EtOH (40:60); Flow rate: 20 mL/min. This resulted in a first eluting stereoisomer (Compound 59A, 6.3 mg, Retention Time 3.639 min) and a second eluting stereoisomer (Compound 59B, 4.3 mg, Retention Time 6.608 min).

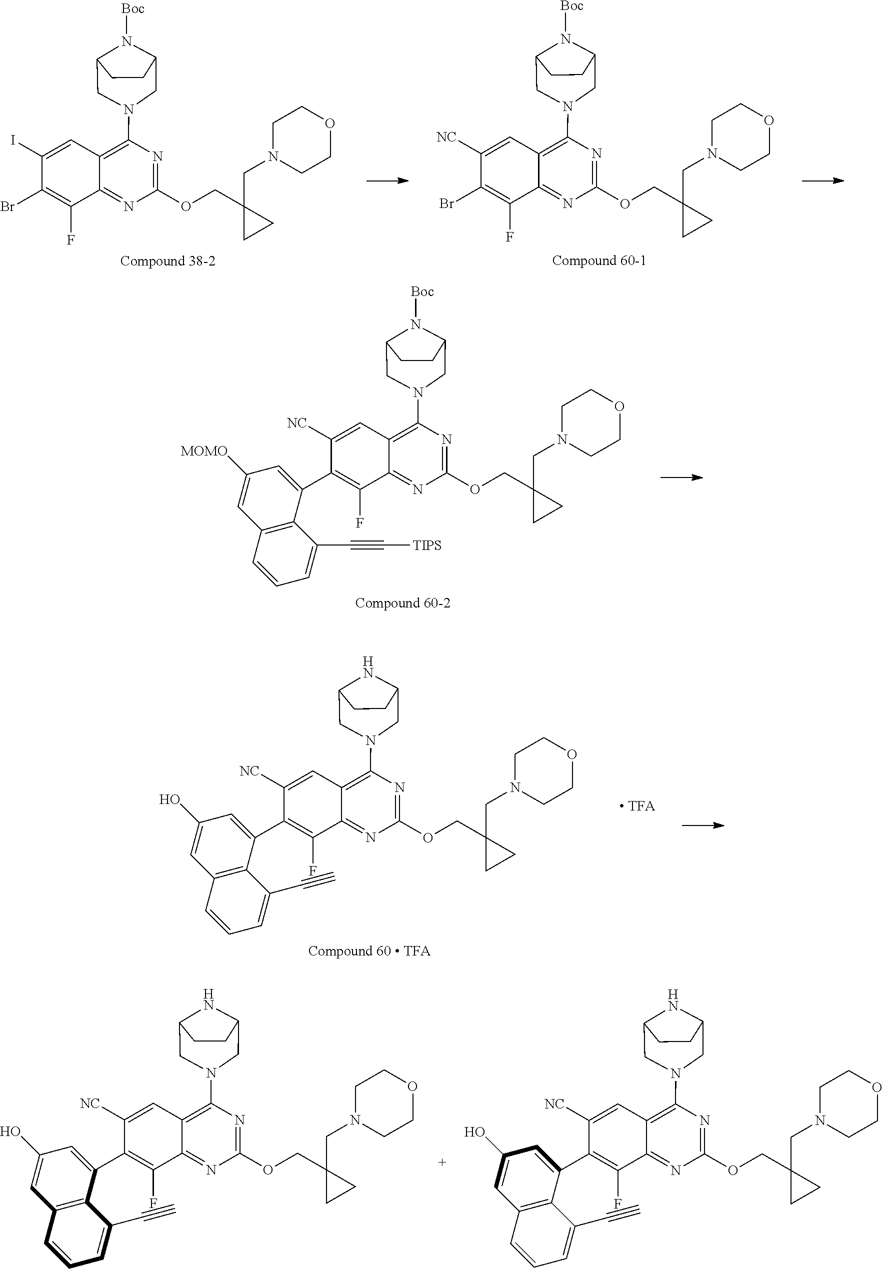

A mixture of Compound 38-2 (300 mg, 0.41 mmol), zinc cyanide (77 mg, 0.66 mmol), Pd(PPh₃)₄ (64 mg, 0.055 mmol) and DMF (8 mL) was stirred overnight at 100° C. under nitrogen atmosphere, and then filtered. The filtrate was concentrated under reduced pressure to obtain a residue which was purified with Pre-TLC (Hex:EA=2:1, v/v) to afford Compound 60-1 (222 mg, 0.35 mmol). MS m/z: 631/633 [M+H]⁺.

Cs₂CO₃ (241 mg, 0.740 mmol) and cataCXium A Pd G₃ (26 mg, 0.036 mmol) were added to a solution of Compound 60-1 (222 mg, 0.35 mmol), INT C12 (210 mg, 0.43 mmol) in toluene (4 mL) and water (1 mL). The reaction mixture was stirred overnight at 100° C. under nitrogen atmosphere, diluted with water (20 mL) and extracted with EA (2×20 mL). The organic layers were combined, dried over Na₂SO₄ and concentrated under reduced pressure to obtain a residue which was purified with Pre-TLC (Hex:EA=3:1, v/v) to afford Compound 60-2 (226 mg, 0.25 mmol). MS m/z: 919 [M+H]⁺.

A solution of Compound 60-2 (226 mg, 0.25 mmol), and HCl (1 mL, 1 M in 1,4-dioxane) in MeCN (3 mL) was stirred at R.T for 1 h, and then concentrated under reduced pressure to obtain a residue. The residue was diluted with sat. NaHCO₃ (20 mL) and extracted with EA (2×20 mL). The organic layers were combined, dried over Na₂SO₄ and concentrated under reduced pressure to obtain a residue. A mixture of the residue, CsF (0.44 g, 2.90 mmol) and DMF (3 mL) was stirred at 40° C. for 2 hours under nitrogen atmosphere. The mixture was diluted with EA (30 mL), and washed with sat. NaHCO₃ (20 mL) and brine (20 mL). The collected organic layer was dried over Na₂SO₄, concentrated, and purified with Prep-HPLC (C18 column; eluent A: 0.1% TFA in water; eluent B: CH₃CN; Gradient: 15% B to 40% B in 40 min at a flow rate of 60 mL/min; 240 nm) to afford the TFA salt of Compound 60 (163.9 mg, 0.194 mmol). MS m/z: 619 [M+H]⁺.

The enantioseparation of the Compound 60 was performed by chiral-HPLC with the following condition: CHIRAL ART Amylose-SA column (2 cm×25 cm, 5 um) on Prep-HPLC-Gilson; Mobile phase: Hex (1% IPA·M)/EtOH (50:50); Flow rate: 20 mL/min. This resulted in a first eluting stereoisomer (Compound 60A, 29.1 mg, Retention Time 4.393 min) and a second eluting stereoisomer (Compound 60B, 20 mg, Retention Time 7.424 min).

### Example 30

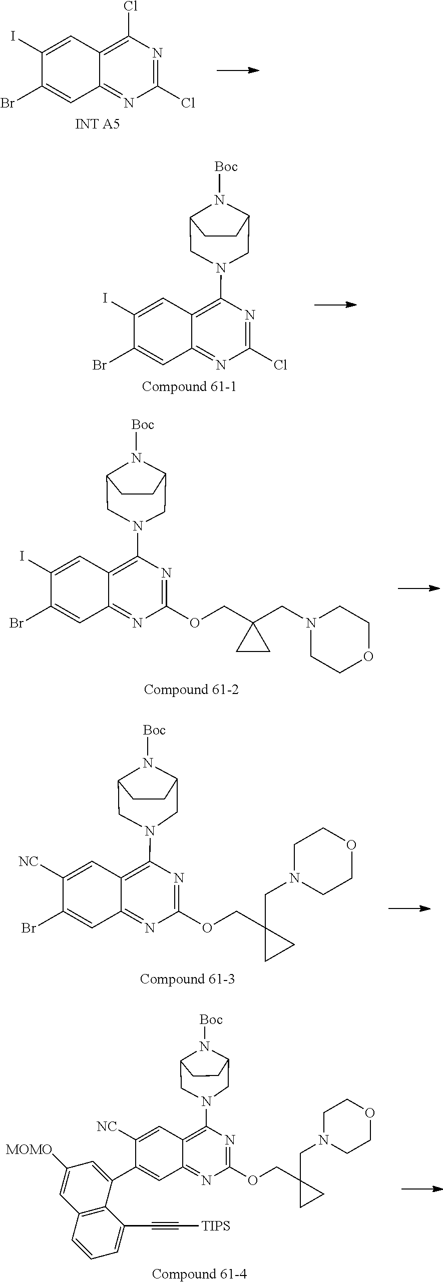

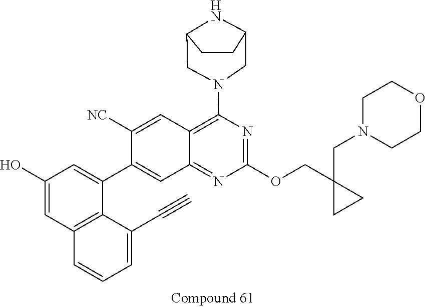 [SMILES: C#Cc1cccc2cc(O)cc(-c3cc4nc(OCC5(CN6CCOCC6)CC5)nc(N5CC6CCC(C5)N6)c4cc3C#N)c12]

DIEA (1.83 g, 14.16 mmol) was added dropwise to a solution of INT A5 (2.99 g, 7.40 mmol) and tert-butyl 3,8-diazabicyclo[3.2.1]octane-8-carboxylate (1.43 g, 6.74 mmol) in DCM (30 mL) at 0° C. The reaction mixture was stirred at R.T for 2 hrs under nitrogen atmosphere, concentrated under reduced pressure to obtain a residue. The mixture of the residue and EA (200 mL) was washed with brine (2×200 mL), dried over Na₂SO₄ and concentrated under reduced pressure to obtain a crude product. The crude product was slurried with Hex/EA (90 mL, 10:1, v/v), filtered, and then dried to afford Compound 61-1 (3.10 g, 5.35 mmol) as a brown solid. MS m/z: 579/601 [M+H]⁺.

A mixture of Compound 61-1 (1.50 g, 2.59 mmol), INT B2 (0.54 g, 3.15 mmol), triethylenediamine (0.18 g, 1.60 mmol), Cs₂CO₃ (1.26 g, 3.87 mmol) and THF/DMF (12 mL, 1:1, v/v) was stirred at R.T for 2 days under nitrogen atmosphere, diluted with water (20 mL) and extracted with EA (2×20 mL). The organic layers were combined, dried over Na₂SO₄ and concentrated under reduced pressure to obtain a residue. The residue was purified by silica gel chromatography (eluting with 20% EA/Hex) to afford Compound 61-2 (1.13 g, 1.58 mmol). MS m/z: 714/716 [M+H]⁺.

A mixture of Compound 61-2 (300 mg, 0.42 mmol), zinc cyanide (77 mg, 0.66 mmol), Pd(PPh₃)₄ (50 mg, 0.043 mmol) and DMF (8 mL) was stirred overnight at 100° C. under nitrogen atmosphere, then filtered and the filtrate concentrated under reduced pressure to obtain a residue which was purified with Pre-TLC (Hex:EA=3:1, v/v) to afford Compound 61-3 (230 mg, 0.32 mmol). MS m/z: 613/615 [M+H]⁺.

Cs₂CO₃ (255 mg, 0.78 mmol) and cataCXium A Pd G₃ (30 mg, 0.041 mmol) were added to a solution of Compound 61-3 (230 mg, 0.32 mmol), INT C12 (214 mg, 0.43 mmol) in toluene (4 mL) and water (1 mL). The reaction mixture was stirred at 100° C. for 8 hrs under nitrogen atmosphere, diluted with water (20 mL) and extracted with EA (2×20 mL). The organic layers were combined, dried over Na₂SO₄ and concentrated under reduced pressure to obtain a residue which was purified with Pre-TLC (Hex:EA=3:1, v/v) to afford Compound 61-4 (188 mg, 0.21 mmol). MS m/z: 901 [M+H]⁺.

A solution of Compound 61-4 (188 mg, 0.21 mmol) and HCl (1 mL, 1 M in 1,4-dioxane) in MeCN (3 mL) was stirred at R.T for 1 h, and then concentrated under reduced pressure to obtain a residue. The residue was diluted with sat. NaHCO₃ (20 mL) and extracted with EA (2×20 mL). The organic layers were combined, dried over Na₂SO₄ and concentrated under reduced pressure to obtain a residue. A mixture of the residue, CsF (0.37 g, 2.44 mmol) and DMF (3 mL) was stirred at 40° C. for 2 hrs under nitrogen atmosphere, and then purified with Prep-HPLC (C18 column; eluent A: 0.1% TFA in water; eluent B: CH₃CN; Gradient: 15% B to 40% B in 40 min at a flow rate of 60 mL/min; 240 nm) to afford the TFA salt of Compound 61 (120.3 mg, 0.145 mmol). MS m/z: 601 [M+H]⁺

### Example 31

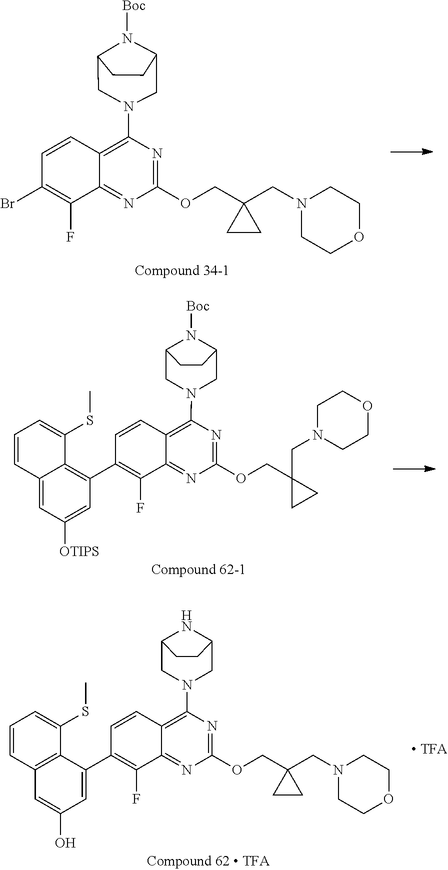

K₃PO₄ (300 mg, 1.41 mmol) and cataCXium A Pd G₃ (34 mg, 0.047 mmol) were added to a solution of Compound 34-1 (202 mg, 0.33 mmol), INT C18 (199 mg, 0.42 mmol) in toluene (6 mL) and water (1.5 mL). The reaction mixture was stirred at 100° C. for 4 hrs under nitrogen atmosphere, diluted with water (20 mL) and extracted with EA (2×20 mL). The organic layers were combined, dried over Na₂SO₄ and concentrated under reduced pressure to obtain a residue which was purified with Pre-TLC (Hex:EA=1.5:1, v/v) to afford Compound 62-1 (263 mg, 0.30 mmol) as a light yellow solid. MS m/z: 872[M+H]⁺.

A solution of Compound 62-1 (252 mg, 0.29 mmol) and HCl (0.8 mL, 1 M in 1,4-dioxane) in DCM (2 mL) was stirred at R.T for 2 hrs, and then concentrated in vacuum to obtain a residue. A mixture of the residue and sat. NaHCO₃ (20 mL) was extracted with EA (2×20 mL), and the organic layers were combined, dried over Na₂SO₄ and concentrated under reduced pressure to obtain a crude product. Tetrabutylammonium fluoride (0.7 mL, 1 M in THF) was added to a solution of the crude product in THF (2 mL). The resulting mixture was stirred at R.T for 30 mins under nitrogen atmosphere, and then purified with Prep-HPLC (C18 column; eluent A: 0.1% TFA in water; eluent B: CH₃CN; Gradient: 10% B to 35% B in 40 min at a flow rate of 60 mL/min; 245 nm) to afford the TFA salt of Compound 62 (203.2 mg, 0.241 mmol). MS m/z: 616 [M+H]⁺.

### Example 32

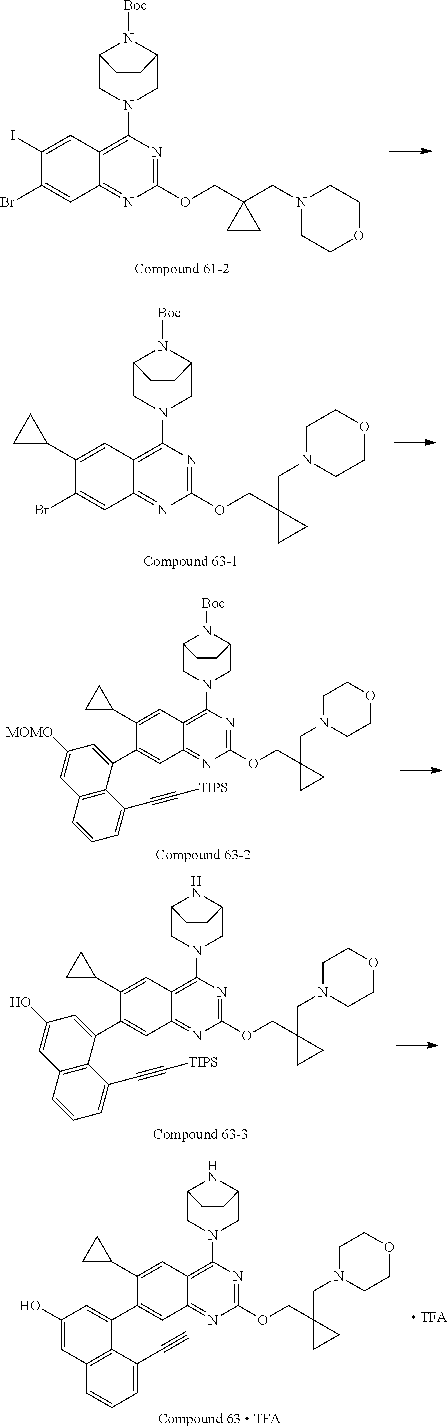

K₂CO₃ (150 mg, 1.09 mmol), Pd(dppf)Cl₂·CH₂Cl₂ (34 mg, 0.046 mmol) was added to a solution of Compound 61-2 (304 mg, 0.43 mmol), cyclopropylboronic acid (41 mg, 0.48 mmol) in toluene (6 mL) and water (1 mL). The reaction mixture was stirred overnight at 100° C. under nitrogen atmosphere, then filtered and the filtrate was concentrated under reduced pressure to obtain a residue which was purified with Pre-TLC (Hex:EA=1:1, v/v) to afford Compound 63-1 (140 mg, 0.22 mmol). MS m/z: 628/630 [M+H]⁺.

Cs₂CO₃ (153 mg, 0.47 mmol) and cataCXium A Pd G₃ (20 mg, 0.027 mmol) was added to a solution of Compound 63-1 (140 mg, 0.22 mmol) and INT C12 (126 mg, 0.26 mmol) in toluene (4 mL) and water (1 mL) was added. The reaction mixture was stirred overnight at 100° C. under nitrogen atmosphere, diluted with water (20 mL) and extracted with EA (2×20 mL). The organic layers were combined, dried over Na₂SO₄ and concentrated under reduced pressure to obtain a residue which was purified with Pre-TLC (Hex:EA=3:1, v/v) to afford Compound 63-2 (164 mg, 0.18 mmol). MS m/z: 916 [M+H]⁺.

A solution of Compound 63-2 (164 mg, 0.18 mmol) and HCl (1 mL, 1 M in 1,4-dioxane) in MeCN (3 mL) was stirred at R.T for 1 h, and then concentrated under reduced pressure to obtain a residue. The residue was diluted with sat. NaHCO₃ (20 mL) and then extracted with EA (2×20 mL). The organic layers were combined, dried over Na₂SO₄ and concentrated under reduced pressure to afford Compound 63-3 (136 mg, 0.18 mmol). MS m/z: 772 [M+H]⁺.

A mixture of Compound 63-3, CsF (0.31 g, 2.04 mmol) and DMF (3 mL) was stirred overnight at 40° C. under nitrogen atmosphere, and then purified with Prep-HPLC (C18 column; eluent A: 0.1% TFA in water; eluent B: CH₃CN; Gradient: 10% B to 40% B in 40 min at a flow rate of 60 mL/min; 245 nm) to afford the TFA salt of Compound 63 (70.5 mg, 0.084 mmol). MS m/z: 616 [M+H]⁺.

### Example 33

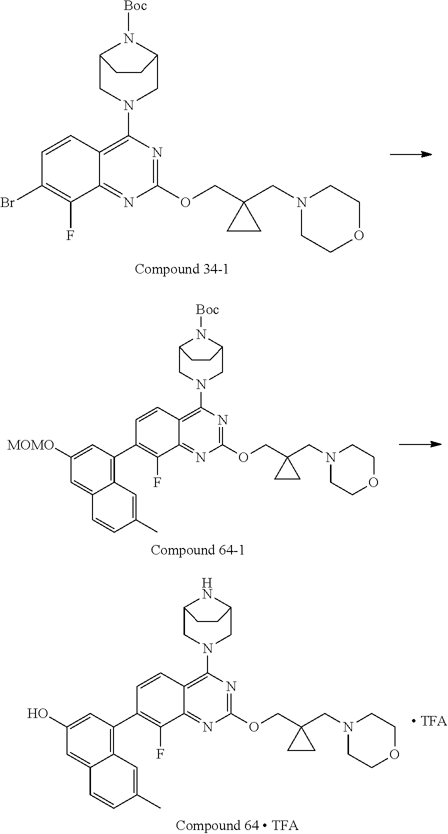

Na₂CO₃ (67 mg, 0.63 mmol) and Pd(PPh₃)₄(45 mg, 0.039 mmol) were added to a solution of Compound 34-1 (157 mg, 0.26 mmol), INT C19 (103 mg, 0.31 mmol) in 1,4-dioxane (4 mL) and water (1 mL). The reaction mixture was stirred at 75° C. for 4 hrs under nitrogen atmosphere, diluted with water (20 mL) and extracted with EA (2×20 mL). The organic layers were combined, dried over Na₂SO₄ and concentrated under reduced pressure to obtain a residue which was purified with Pre-TLC (Hex:EA=1.5:1, v/v) to afford Compound 64-1 (196 mg, 0.27 mmol) as a light yellow solid. MS m/z: 728[M+H]⁺.

A mixture of Compound 64-1 (196 mg, 0.27 mmol), HCl (2 mL, 1 M in 1,4-dioxane) and MeCN (4 mL) was stirred at R.T for 1 h, and then concentrated in vacuum to obtain a residue. The residue was diluted with saturated aq. NaHCO₃ (20 mL) and extracted with EA (2×20 mL). The organic layers were combined, dried over Na₂SO₄ and concentrated under reduced pressure to obtain a crude product which was purified by Prep-HPLC (C18 column; eluent A: 0.1% TFA in water; eluent B: CH₃CN; Gradient: 15% B to 40% B in 35 min at a flow rate of 60 mL/min; 280 nm) to afford the TFA salt of Compound 64 (107.8 mg, 0.19 mmol). MS m/z: 584[M+H]⁺.

### Example 34

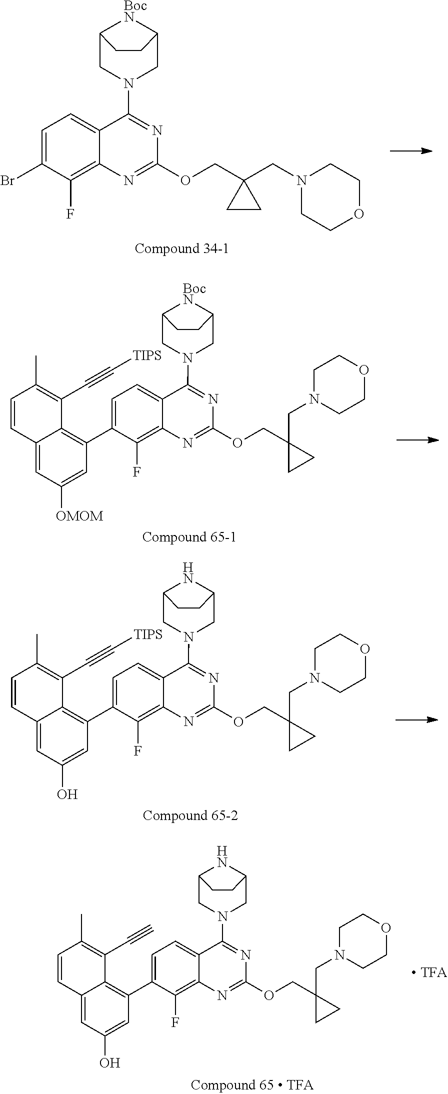

A mixture of Compound 34-1 (85 mg, 140.14 μmol), INT C21 (74 mg, 145.50 μmol), cataCXium A Pd G₃ (18 mg, 24.71 μmol), K₃PO₄ (117 mg, 551.19 μmol) in toluene (8 mL) and water (2.0 mL) was stirred at 100° C. for 2.5 hrs under nitrogen atmosphere, diluted with EA (50 mL), and then washed with water (2×30 mL). The organic layer was dried over Na₂SO₄ and concentrated under reduced pressure to obtain a residue which was purified with Pre-TLC (MeOH:DCM=1:20, v/v) to afford Compound 65-1 (109 mg, 120.01 μmol, 85.6% yield). MS m/z: 908 [M+H]⁺.

A mixture of Compound 65-1 (109 mg, 120.01 μmol), HCl/1,4-dioxane (4 M in 1,4-dioxane, 1.5 mL) and CH₃CN (6 mL) was stirred at R.T for 1 h, and then concentrated under reduced pressure to obtain a residue containing Compound 65-2 which was used in next step directly. MS m/z: 764[M+H]⁺.

A mixture of CsF (0.22 g, 1.44 mmol), the residue containing Compound 65-2 and DMF (5 mL) was stirred at 40° C. for 16 hrs, and then concentrated under reduced pressure and the residue was purified by Pre-HPLC (Daisogel-C18 column, 50*250 mm, 10 mi; eluent A: 0.1% TFA in water; eluent B: CH₃CN; Gradient: 15% B to 35% B in 35 min at a flow rate of 60 mL/min; 280 nm) to afford the TFA salt of Compound 65 (60.2 mg, 83.40 μmol, 69.4% yield). MS m/z: 608[M+H]⁺.

### Example 35

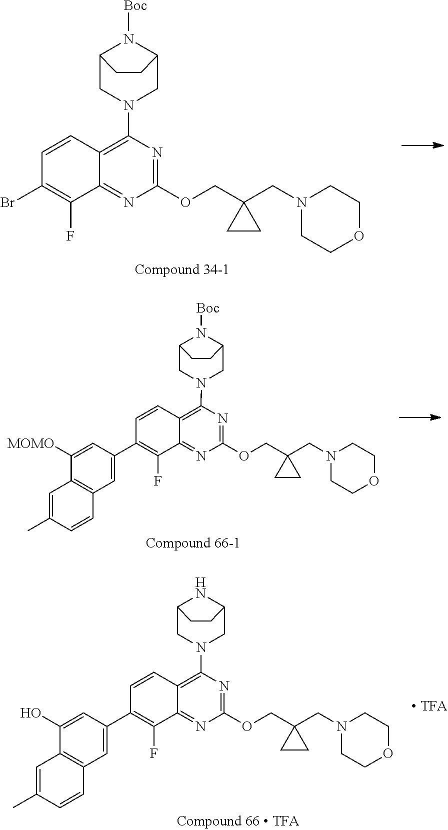

Na₂CO₃ (60 mg, 0.57 mmol) and Pd(PPh₃)₄(31 mg, 0.027 mmol) were added to a mixture of Compound 34-1 (120 mg, 0.20 mmol), INT C20 (94 mg, 0.29 mmol), 1,4-dioxane (4 mL) and water (1 mL). The reaction mixture was stirred at 75° C. for 3 hrs under nitrogen atmosphere, diluted with water (20 mL) and extracted with EA (2×20 mL). The organic layers were combined, dried over Na₂SO₄ and concentrated under reduced pressure. The residue was purified by Pre-TLC (Hex:EA=1:1, v/v) to afford Compound 66-1 (145 mg, 0.20 mmol) as a light yellow solid. MS m/z: 728[M+H]⁺.

A solution of Compound 66-1 (145 mg, 0.20 mmol) and HCl (1 mL, 1 M in 1,4-dioxane) in MeCN (3 mL) was stirred at R.T for 1 h, and then concentrated under reduced pressure to obtain a residue. The residue was diluted with saturated NaHCO₃ (20 mL) and extracted with EA (2×20 mL). The organic layers were combined, dried over Na₂SO₄ and concentrated under reduced pressure to obtain a crude product which was purified by Prep-HPLC (C18 column; eluent A: 0.1% TFA in water; eluent B: CH₃CN; Gradient: 15% B to 40% B in 35 min at a flow rate of 60 mL/min; 280 nm) to afford the TFA salt of Compound 66 (65.3 mg, 0.080 mmol). MS m/z: 584[M+H]⁺.

### Example 36

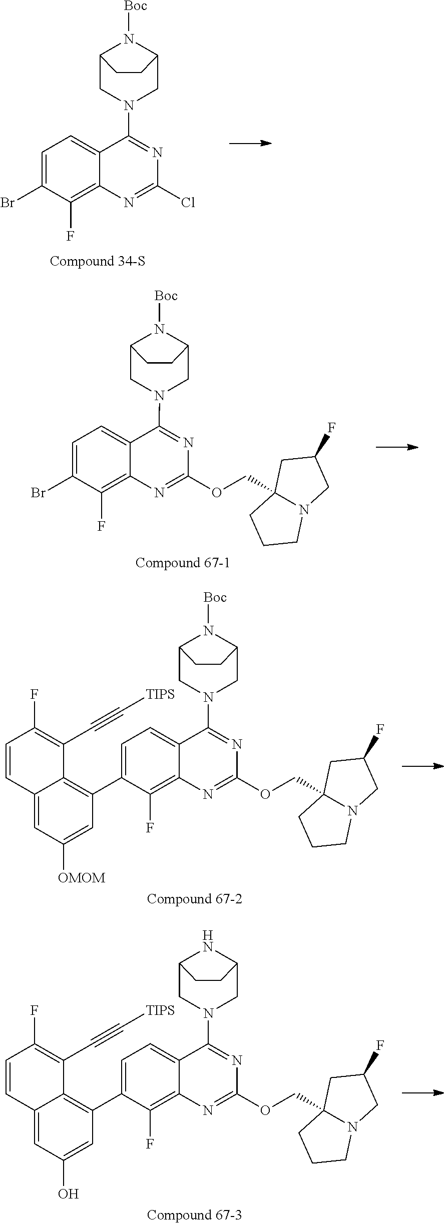

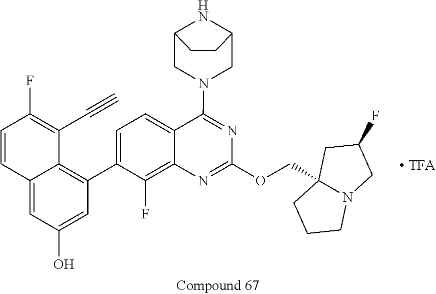 [SMILES: C#Cc1c(F)ccc2cc(O)cc(-c3ccc4c(N5CC6CCC(C5)N6)nc(OC[C@@]56CCCN5C[C@H](F)C6)nc4c3F)c12]

To a solution of Compound 34-S (1.54 g, 3.26 mmol) and ((2R,7aS)-2-fluorotetrahydro-1H-pyrrolizin-7a(5H)-yl)methanol (0.89 g, 5.59 mmol) in DMSO (12 mL) was added KF (0.55 g, 9.47 mmol) at room temperature. The reaction mixture was stirred at 100° C. for 17 h, cooled to room temperature, diluted with EA (50 mL) and water (50 mL). The organic phase was collected and concentrated under reduced pressure. The residue was purified by silica gel chromatography to afford Compound 67-1 (0.93 g, 47.9%). LCMS [M+1]=594.

To a solution of Compound 67-1 (446 mg, 0.75 mmol) and INT C13 (546 mg, 1.07 mmol) in 1,4-dioxane (4 mL) and water (1 mL) were added Cs₂CO₃ (492 mg, 1.51 mmol) and Pd(dppf)Cl₂ (56 mg, 0.077 mmol). The reaction mixture was purged with N₂ for three times and stirred at 100° C. for 3 h, cooled to room temperature, diluted with EA (50 mL) and water (50 mL). The organic phase was collected and concentrated under reduced pressure. The residue was prepared by prep-TLC to afford Compound 67-2 (215 mg, 32.5%). LCMS [M+1]=900.

To a solution of Compound 67-2 (208 mg, 0.24 mmol) in 1,4-dioxane (5 mL) was added HCl/1,4-dioxane (4 M, 5 mL). The reaction mixture was stirred for 2 h at room temperature, and concentrated under reduced pressure. The obtained Compound 67-3 (crude) was used directly without purification. LCMS [M+1]=756.

To a solution of Compound 67-3 (crude) in DMF (2 mL) was added CsF (0.18 g, 1.19 mmol). The reaction mixture was stirred at room temperature for 2 h, and then filtered to collect the filtrate. CsF (0.18 g, 1.19 mmol) was mixed with the filtrate, and the mixture was stirred for 18 h. MeOH (5 mL) was added, and the mixture was filtered. The filtrate was concentrated under reduced pressure. The residue was purified by Prep-HPLC (C18 column, 50×250 mm, 10 um; A: 0.1% TFA in water, B: CH₃CN, Gradient: 10% B to 40% B in 40 min at a flow rate of 60 mL/min, 220 nm) to afford the TFA salt of Compound 67 (crude, Compound 67. TFA) as a yellow solid. The crude salt was freed by Et₃N to afford a solution of free base, and then purified by chiral-HPLC separation using a CHIRAL ART Cellulose-SC column on Prep-HPLC-Gilson eluting with Hex (0.1% IPA·M)/EtOH (50:50) at a flow rate of 20 mL/min to afford Compound 67 (59.8 mg, 43.6% for two steps). LCMS [M+1]=600. ¹H NMR (400 MHz, CD₃OD) δ 7.82 (dd, J=9.1, 5.8 Hz, 1H), 7.75 (d, J=8.5 Hz, 1H), 7.33-7.25 (m, 2H), 7.21 (dd, J=8.6, 6.8 Hz, 1H), 7.09 (d, J=2.5 Hz, 1H), 5.44-5.19 (m, 1H), 4.50 (t, J=11.0 Hz, 2H), 4.24 (m, 2H), 3.69-3.54 (m, 4H), 3.28-3.12 (m, 4H), 3.08-2.94 (m, 1H), 2.42-2.09 (m, 3H), 2.06-1.94 (m, 2H), 1.94-1.80 (m, 5H).

### Example 37

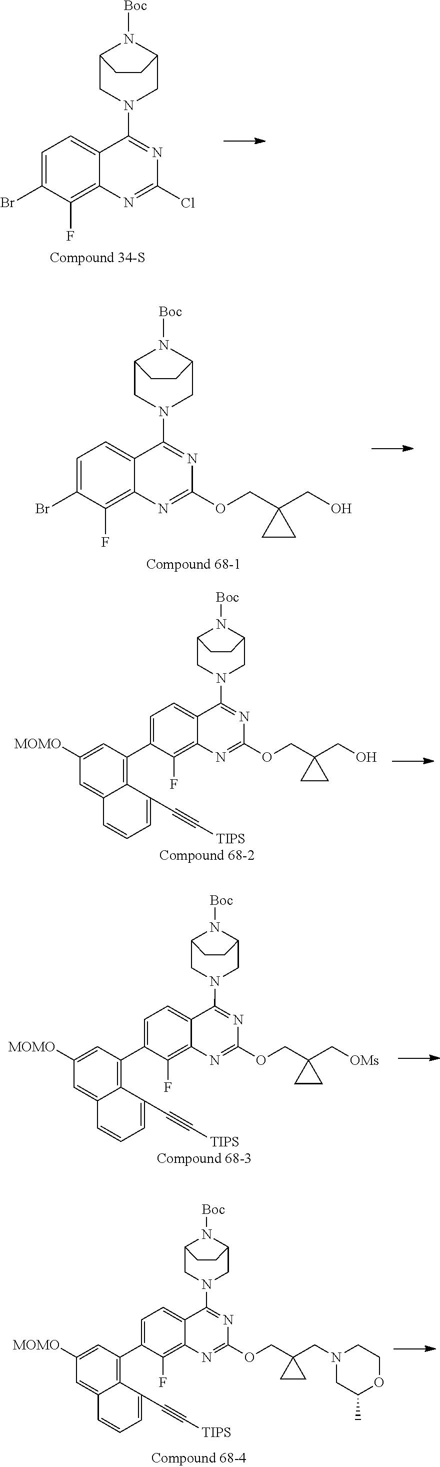

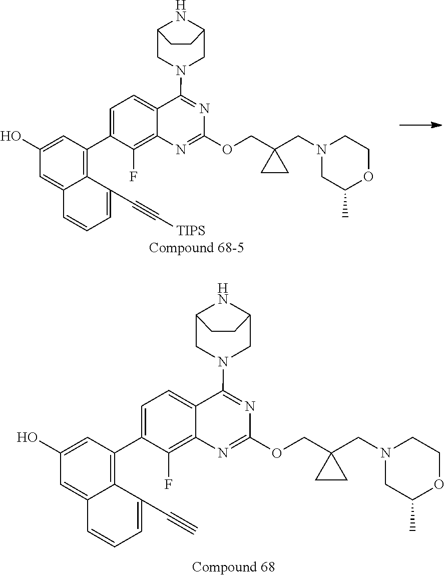

Compound 34-S (6.0 g, 12.7 mmol, 1.0 eq.), cyclopropane-1,1-diyldimethanol (3.9 g, 38.1 mmol, 3.0 eq.), triethylenediamine (1.42 g, 12.7 mmol, 1.0 eq.) and Cs₂CO₃ (12.4 g, 38.1 mmol, 3.0 eq.) were added successively into a mixed solution of THF and DMF (V_{THF}/V_{DMF}=1:1, 120 mL). The reaction mixture was stirred overnight at R.T, poured into water (60 mL) and extracted with EtOAc (60 mL). The organic layer was washed with brine (30 mL), dried with anhydrous Na₂SO₄, and concentrated to obtain a residue. The residue was slurried with EtOAc and then filtered to afford Compound 68-1 (5.2 g, yield 76.1%) as a white solid. LCMS: 537 ([M+H]⁺).

Compound 68-1 (5.2 g, 9.7 mmol, 1.0 eq.), INT C12 (7.17 g, 14.5 mmol, 1.5 eq.), K₃PO₄ (6.15 g, 29.0 mmol, 3.0 eq.), cataCxium A Pd G₃ (1.06 g, 1.45 mmol, 0.15 eq.) were added successively into a mixed solution of THF and water (V_{THF}/V_{water}=5:1, 60 mL) at R.T. The reaction mixture was purged with argon for 10 mins, stirred at 60° C. for 3 hrs, cooled to R.T, poured into water (60 mL) and extracted with EtOAc (60 mL×2). The organic layers were combined, washed with brine (30 mL), dried with anhydrous Na₂SO₄, and then concentrated to obtain a residue which was purified with silica gel chromatography (eluting with petroleum ether/EtOAc=3:1) to afford Compound 68-2 (3.2 g, yield 40.0%) as a yellow solid. LCMS: 825 ([M+H]⁺).

A mixture of Compound 68-2 (3.2 g, 3.88 mmol, 1.0 eq.), MsCl (533 mg, 4.65 mmol, 1.2 eq.), TEA (1.17 g, 11.6 mmol, 3.0 eq.) and DCM (32 mL) was stirred for 2 hrs at R.T, poured into water (30 mL) and extracted with DCM (20 mL). The organic layer was washed with brine (20 mL), dried with anhydrous Na₂SO₄, and concentrated to obtain a crude product containing Compound 68-3 (3.2 g, yield 91.3%) as a yellow solid which was used directly for next step without purification. LCMS: 903 ([M+H]⁺).

A mixture of (R)-2-methylmorpholine (150 mg, 0.17 mmol, 2.0 eq.), TEA (84.0 mg, 0.85 mmol, 5.0 eq.) and DMF (2 mL) was stirred at R.T for 2 hrs, and then Compound 68-3 (150.0 mg, 0.17 mmol, 1.0 eq.) was added. The reaction mixture was stirred for 2 hrs at 80° C., cooled to R.T, poured into water (5 mL) and extracted with EtOAc (5 mL×2). The organic layers were combined, washed with brine (5 mL), dried with anhydrous Na₂SO₄, and concentrated to obtain a residue which was purified with Pre-TLC (eluting with ether/EtOAc=3:1) to afford Compound 68-4 (80 mg, yield 40.0%) as yellow oil. LCMS: 908 ([M+H]⁺).

A solution of Compound 68-4 (80 mg, 0.07 mmol, 1.0 eq.), HCl/Dioxane (0.7 mL) and MeCN (2 mL) was stirred at R.T for 1 h, and then concentrated under reduced pressure to afford a crude product containing Compound 68-5 (80 mg) as a yellow solid. LCMS: 764.00 ([M+H]⁺).

A mixture of crude product containing Compound 68-5 (80 mg), CsF (158 mg, 1.0 mmol, 10.0 eq.) and DMF (2 mL) was stirred overnight at 80° C., and then filtered. The filtrate was purified with Prep-HPLC to afford Compound 68 (6 mg, the yield of the above two steps is 11.3%) as a white solid. ¹H NMR (400 MHz, CD₃OD-d₄): δ 7.80-7.74 (m, 2H), 7.47 (d, J=0.8 Hz, 1H), 7.39-7.37 (m, 1H), 7.27-7.26 (m, 2H), 7.03 (d, J=2.4 Hz, 1H), 4.54-4.50 (m, 2H), 4.42-4.40 (m, 2H), 3.97-3.95 (m, 2H), 3.70-3.66 (m, 1H), 3.66-3.61 (m, 2H), 3.60-3.59 (m, 2H), 2.98-2.95 (m, 2H), 2.88 (s, 1H), 2.52-2.50 (m, 1H), 2.46-2.45 (m, 1H), 2.14-2.04 (m, 6H), 1.09 (t, J=6.4 Hz, 3H), 0.73-0.70 (m, 2H), 0.51-0.49 (m, 2H). LCMS: 608.1 ([M+H]⁺).

### Example 38

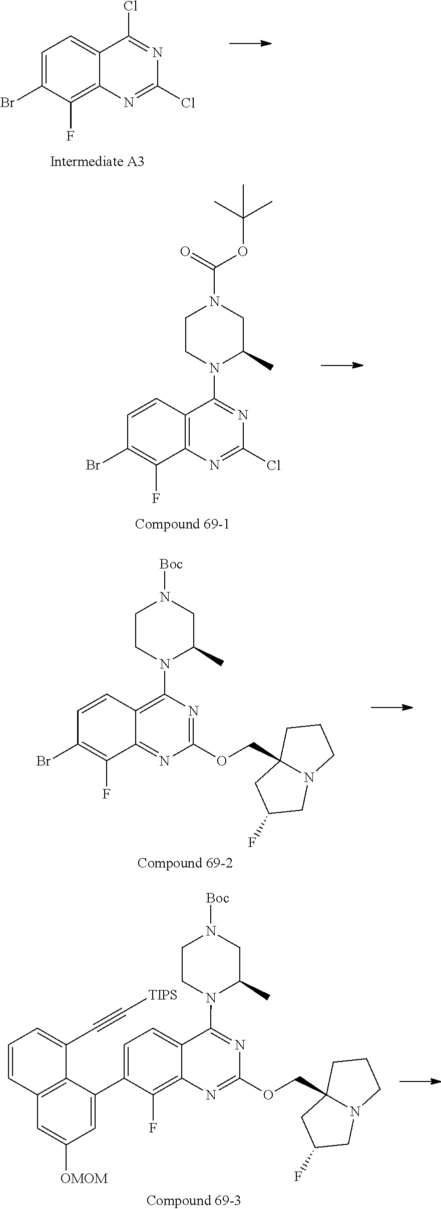

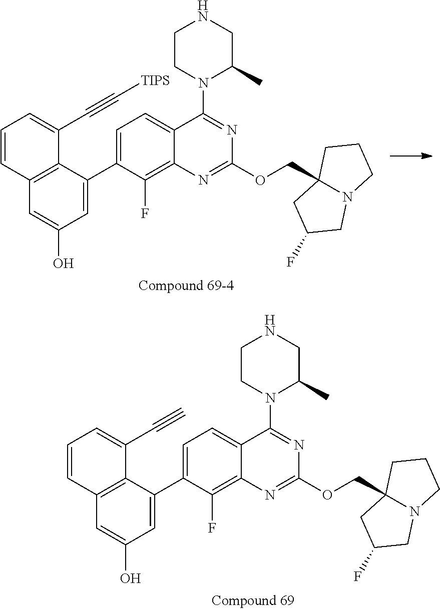

To a solution of INT A3 (2.02 g, 6.83 mmol) and tert-butyl (R)-3-methylpiperazine-1-carboxylate (1.22 g, 6.09 mmol) in DCM (20 mL) was added DIEA (1.74 g, 13.46 mmol) at room temperature. The reaction mixture was stirred for 18 h, then water (20 mL) was added. The organic phase was collected, and concentrated under reduced pressure. The obtained residue was suspended in EA (10 mL) and Hex (90 mL), filtered and the filter cake was collected and dried to afford Compound 69-1 as an off-white solid (2.74 g, 97.8%).

To a solution of Compound 69-1 (1.74 g, 3.78 mmol) and ((2R,7aS)-2-fluorotetrahydro-1H-pyrrolizin-7a(5H)-yl)methanol (0.89 g, 5.59 mmol) in DMSO (12 mL) was added KF (0.42 g, 7.23 mmol) at room temperature. The reaction mixture was stirred at 105° C. for 25 h, cooled to room temperature. The reaction mixture was diluted with EA (50 mL) and water (50 mL). The organic phase was collected and concentrated under reduced pressure. The residue was purified by silica gel chromatography (EA/Hex=1/4-2/1, v/v) to afford Compound 69-2 (1.16 g, 52.4%). LCMS [M+1]=582.

To a solution of Compound 69-2 (222 mg, 0.38 mmol) and INT C12 (245 mg, 0.50 mmol) in 1,4-dioxane (4 mL) and water (1 mL) were added Na₂CO₃ (275 mg, 2.59 mmol) and Pd(dppf)Cl₂ (33 mg, 45.10 μmol). The reaction mixture was purged with nitrogen for three times and stirred at 100° C. for 3.5 h, cooled to room temperature and filtered. The collected filtrate was concentrated under reduced pressure. The residue was prepared by Prep-TLC (eluted with EA) to afford Compound 69-3 (157 mg, 47.3%). LCMS [M+1]=870.

To a solution of Compound 69-3 (159 mg, 0.18 mmol) in 1,4-dioxane (4 mL) was added HCl/1,4-dioxane (4 M, 4 mL). The reaction mixture was stirred for 2 h at room temperature, and concentrated under reduced pressure. The obtained Compound 69-4 (crude) was used directly in next step without further purification. LCMS [M+1]=726.

To a solution of Compound 69-4 (crude) in DMF (2 mL) was added CsF (0.38 g, 2.50 mmol). The reaction mixture was stirred at room temperature for 2.5 h, then filtered and the filtrate was collected. CsF (0.38 g, 2.50 mmol) was mixed with the filtrate, and the mixture was stirred for another 20 h. The mixture was purified by Prep-HPLC (Agela Venusil PrepG C18 column, 30*250 mm, 10 μm; A: 0.1% TFA in water, B: CH₃CN, Gradient: 10% B to 35% B in 40 min at a flow rate of 40 mL/min, 220 nm) to afford the TFA salt of Compound 69 (Compound 69-TFA, 81.8 mg, 56.1% for two steps) as a yellow solid. LCMS [M+1]=570. ¹H NMR (400 MHz, CD₃OD) δ 7.79 (d, J=7.4 Hz, 1H), 7.71 (dd, J=8.5, 4.2 Hz, 1H), 7.48 (dd, J=7.1, 1.1 Hz, 1H), 7.39-7.33 (m, 1H), 7.29-7.22 (m, 2H), 7.04 (d, J=2.6 Hz, 1H), 5.43-5.20 (m, 1H), 4.81-4.70 (m, 1H), 4.40-4.11 (m, 3H), 3.74-3.53 (m, 1H), 3.31-3.20 (m, 3H), 3.19-2.97 (m, 4H), 2.96-2.83 (m, 2H), 2.44-2.11 (m, 3H), 2.08-1.83 (m, 3H), 1.53 (dd, J=6.8, 5.0 Hz, 3H).

### Example 39

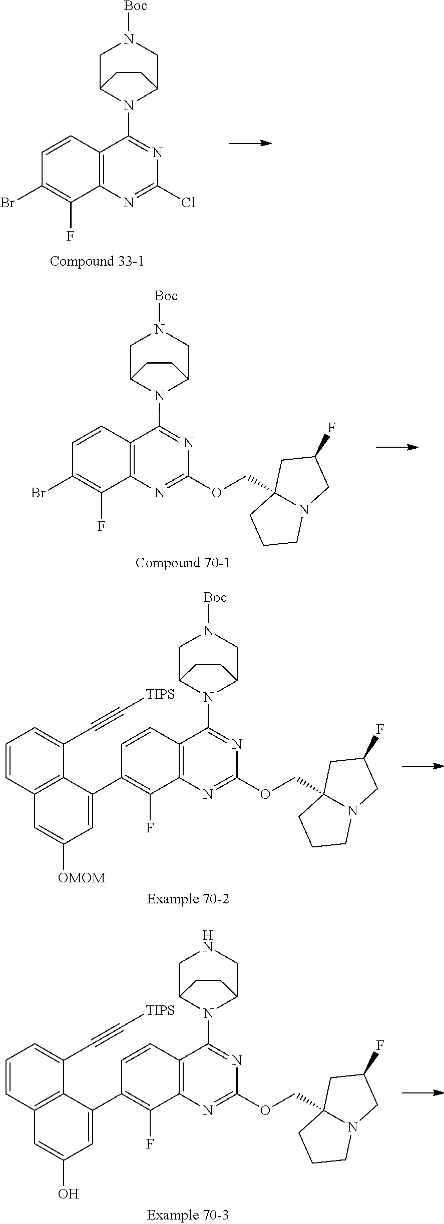

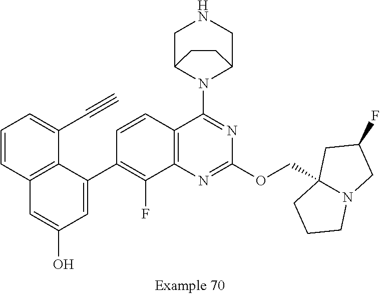 [SMILES: C#Cc1cccc2cc(O)cc(-c3ccc4c(N5C6CCC5CNC6)nc(OC[C@@]56CCCN5C[C@H](F)C6)nc4c3F)c12]

To a suspension of Compound 33-1 (234 mg, 0.50 mmol) and ((2R,7aS)-2-fluorotetrahydro-1H-pyrrolizin-7a(5H)-yl)methanol (137 mg, 0.86 mmol) in DMSO (5 mL) was added KF (90 mg, 1.55 mmol) and the mixture was purged with nitrogen followed by stirring at 120° C. for 48 h. The mixture was diluted with EA (40 mL) and water (30 mL), the organic layer was collected, washed with brine (30 mL), dried over anhydrous Na₂SO₄, filtered and concentrated under reduced pressure. The residue was purified by Pre-TLC (DCM:MeOH=20:1, v/v) to afford Compound 70-1 (192 mg, 0.32 mmol). MS: m/z 594 [M+1]⁺.

To a solution of Compound 70-1 (192 mg, 0.32 mmol), INT 3 (230 mg, 0.47 mmol) and Na₂CO₃ (96 mg, 0.91 mmol) in 1,4-dioxane (8 mL) and water (2 mL) was added PdCl₂(dppf)₂ (43 mg, 0.037 mmol). The mixture was purged with nitrogen followed by stirring at 100° C. for 3 h. Upon completion, the reaction mixture was concentrated under reduced pressure, the residue was diluted with EA (40 mL) and water (30 mL) and the organic layer was separated. The combined organic layers were concentrated under reduced pressure. The residue was purified by Pre-HPLC (0.1% TFA in water/acetonitrile) to afford Compound 70-2 (78 mg, 0.088 mmol). MS: m/z 882 [M+H]⁺.

To a solution of Compound 70-2 (78 mg, 0.088 mmol) in CH₃CN (4 mL) was added HCl (a solution of 1,4-dioxane, 4 M, 1.5 mL). The reaction mixture was stirred at room temperature for 3 h. After completion, the reaction mixture was concentrated under reduced pressure to afford Compound 70-3 (172 mg, crude) which was used in next step without further purification. MS: m/z 738 [M+H]⁺.

To a mixture of Compound 70-3 (172 mg, crude) in DMF (4 mL) was added CsF (188 mg, 1.24 mmol). The mixture was stirred at room temperature for 24 hours. After completion, the mixture was purified by Pre-HPLC (Daisogel-C18 column, 50*250 mm, A: 0.1% TFA in water, B: CH₃CN, Gradient: 15% B to 40% B in 35 min at a flow rate of 60 mL/min, 220 nm) to afford the TFA salt of Compound 70 (Compound 70-TFA, 8.6 mg, 0.015 mmol). MS: m/z 582 [M+H]⁺.

### Example 40

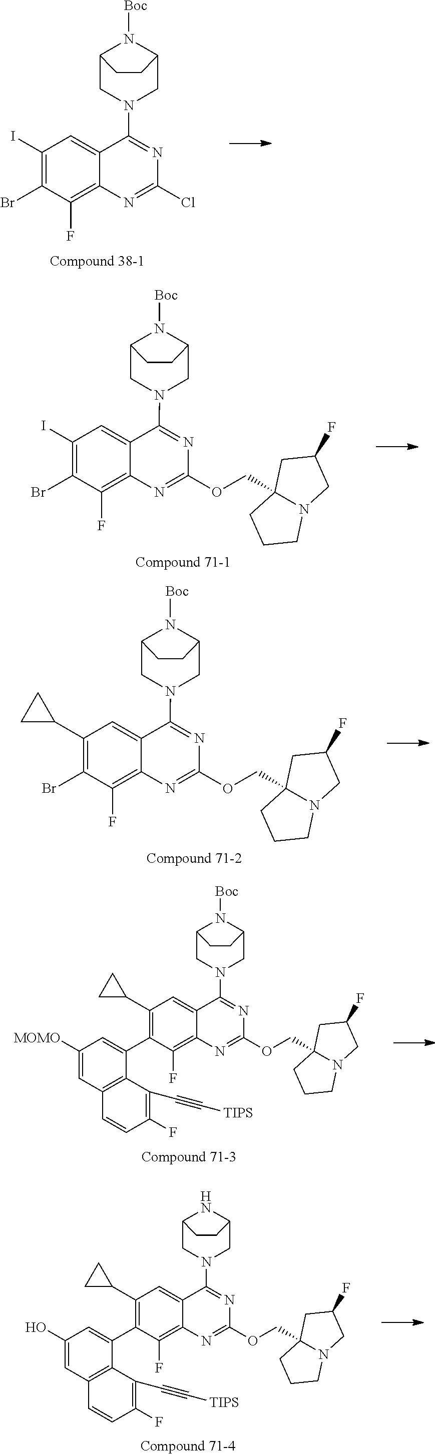

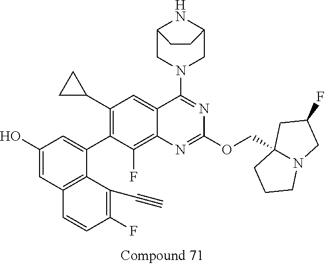 [SMILES: C#Cc1c(F)ccc2cc(O)cc(-c3c(C4CC4)cc4c(N5CC6CCC(C5)N6)nc(OC[C@@]56CCCN5C[C@H](F)C6)nc4c3F)c12]

A mixture of Compound 38-1 (4.15 g, 6.94 mmol), ((2R,7aS)-2-fluorotetrahydro-1H-pyrrolizin-7a(5H)yl)methanol (2.79 g, 17.53 mmol), KF (1.79 g, 30.81 mmol) and DMSO (30 mL) was stirred at 120° C. for 16 hrs under nitrogen atmosphere, water (80 mL) was added, and then filtered. The filtrate and the eluent of the filter cake which was washed with Hex:EA=2:1 (60 mL) were combined and concentrated in vacuum to obtain a residue. The residue was purified by silica gel column chromatography eluted with Hex/EA (2:1 to 1:100, v/v) to afford the desired product Compound 71-1 (2.86 g, 3.97 mmol). MS m/z: 720 [M+H]⁺.

K₃PO₄ (2.11 g, 9.94 mmol) and Pd(dppf)Cl₂ (0.31 g, 0.42 mmol) was added to a solution of Compound 71-1 (2.86 g, 3.97 mmol), cyclopropylboronic acid (3.41 g, 39.70 mmol) dissolved in 1,4-dioxane (24 mL) and water (6 mL). The reaction mixture was stirred at 70° C. for 3.5 hours under nitrogen atmosphere, diluted with water (40 mL) and extracted with EA (2×30 mL). The combined organic layers were dried over Na₂SO₄ and concentrated under vacuum to obtain a residue which was purified by silica gel column chromatography eluted with Hex/EA (3:1˜1:100, v/v) to afford Compound 71-2 (1.70 g, 2.68 mmol). MS m/z: 634 [M+H]⁺.

Potassium trimethylsilanolate (184 mg, 1.43 mmol), ruphos Pd G₃ (41 mg, 0.049 mmol) and ruphos (39 mg, 0.084 mmol) was added to a solution of Compound 71-2 (417 mg, 0.66 mmol) and INT C13 (513 mg, 1.00 mmol) dissolved in 1,4-dioxane (10 mL) and water (2.5 mL). The reaction mixture was stirred at 100° C. for 5 hrs under nitrogen atmosphere, diluted with EA (40 mL) and washed with brine (40 mL). The organic layer was dried over Na₂SO₄ and concentrated under vacuum to obtain a residue which was purified by Prep-TLC to afford Compound 71-3 (125 mg, 0.13 mmol). MS m/z: 940 [M+H]⁺.

A solution of Compound 71-3 (125 mg, 0.13 mmol), HCl/dioxane (1.5 mL, 4 mol/L), and ACN (3 mL) was stirred at RT for 1 h, and then concentrated in vacuum to afford a crude containing Compound 71-4. MS m/z: 796 [M+H]⁺.

TABF (1 mL, 1 M in THF) was added to a solution of Compound 71-4 dissolved in DMF (2 ml). The reaction mixture was stirred at 35° C. for 1.5 hrs, diluted with EA (40 mL), washed with saturated aq. K₂CO₃ (40 mL). The organic layer was dried over Na₂SO₄ and concentrated in vacuum to afford a residue which was purified by Prep-HPLC to afford Compound 71 (1.7 mg, 2.7 umol). MS m/z: 640 [M+H]⁺.

### Example 41

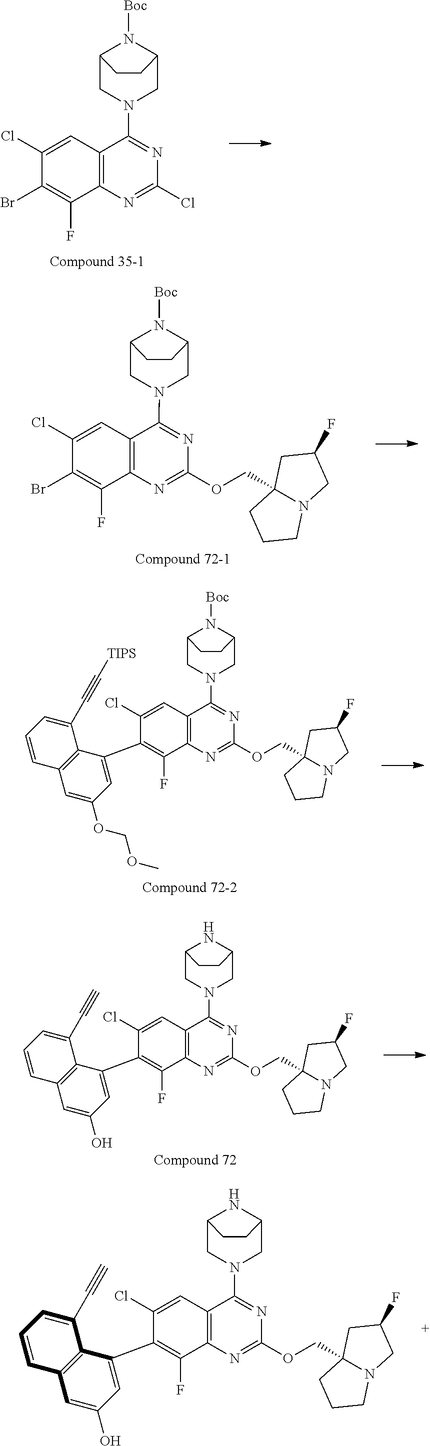

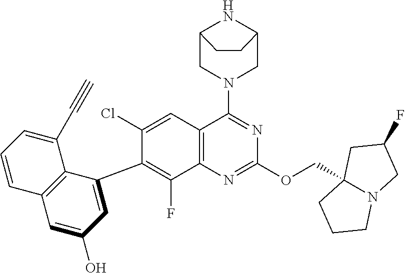

Compound 72-1 was synthesized using Compound 35-1 as a starting material according to the synthetic procedure of Compound 69-1.

Cs₂CO₃ (332 mg, 1.02 mmol) and cataCXium A Pd G3 (33 mg, 0.045 mmol) was added to a solution of Compound 72-1 (201 mg, 0.32 mmol), INT C12 (229 mg, 0.46 mmol) dissolved in toluene (4 mL) and water (1 mL). The reaction mixture was stirred at 100° C. for 4 hrs under nitrogen atmosphere, and then filtered. The filtrate was concentrated under vacuum to obtain a residue which was purified by Pre-TLC (Hex:EA=1:1, v/v) to afford Compound 72-2 as brown oil (145 mg, 0.158 mmol). MS m/z: 917 [M+H]⁺.

A mixture of Compound 72-2 (145 mg, 0.158 mmol), HCl (1 mL, 1 M in dioxane) and CH₃CN (3 mL) was stirred at RT for 1.5 hrs, and then concentrated in vacuum to obtain a crude. The crude was diluted with saturated NaHCO₃ (20 mL) and extracted with EA (2×20 mL). The combined organic layers were dried over Na₂SO₄ and concentrated under vacuum to obtain a residue. The residue was dissolved in DMF (3 mL) and CsF (410 mg, 2.70 mmol) was added. The reaction mixture was stirred at 40° C. for 2 hours under nitrogen atmosphere, diluted with water (20 mL), extracted with EA (2×20 mL), and the collected organic layers were dried over Na₂SO₄ and concentrated under vacuum to obtain a resulting mixture containing Compound 72.

The enantioseparation of the resulting mixture containing Compound 72 was performed by chiral-HPLC with the following condition: Equipment and Column: CHIRALPAK ID column (2 cm×25 cm, 5 um) on Prep-HPLC-Gilson; Mobile phase: Hex (0.1% IPA·M)/EtOH (50:50); Flow Rate: 20 mL/min; Detector Wavelength: 220 nm. This resulted in a first eluting stereoisomer (Compound 72A, 26.9 mg, Retention Time 4.473 min) and a second eluting stereoisomer (Compound 72B, 21.0 mg, Retention Time 5.971 min).

### Example 42

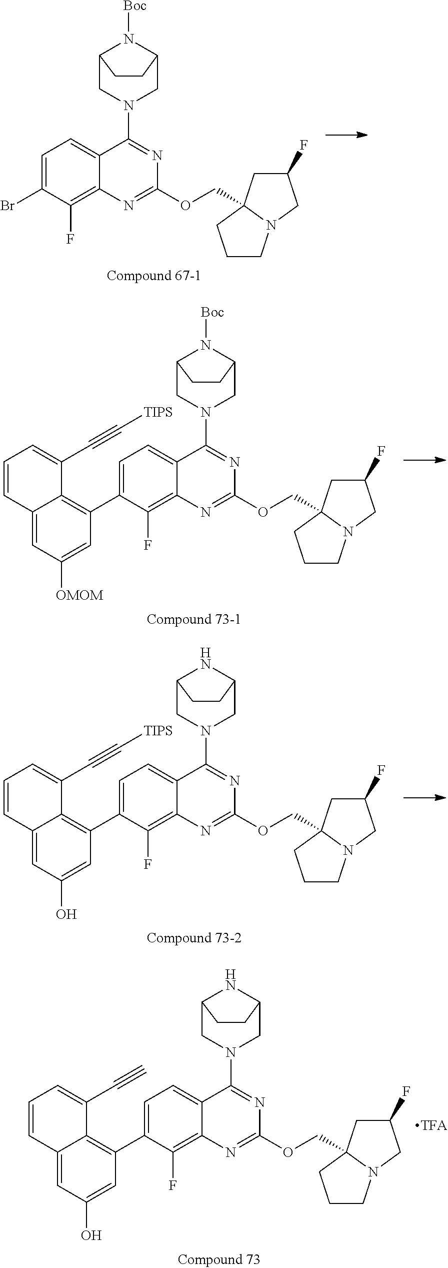

Compound 73 was synthesized using Compound 67-1 as starting material according to the synthetic procedure of Compound 67.

### Example 43

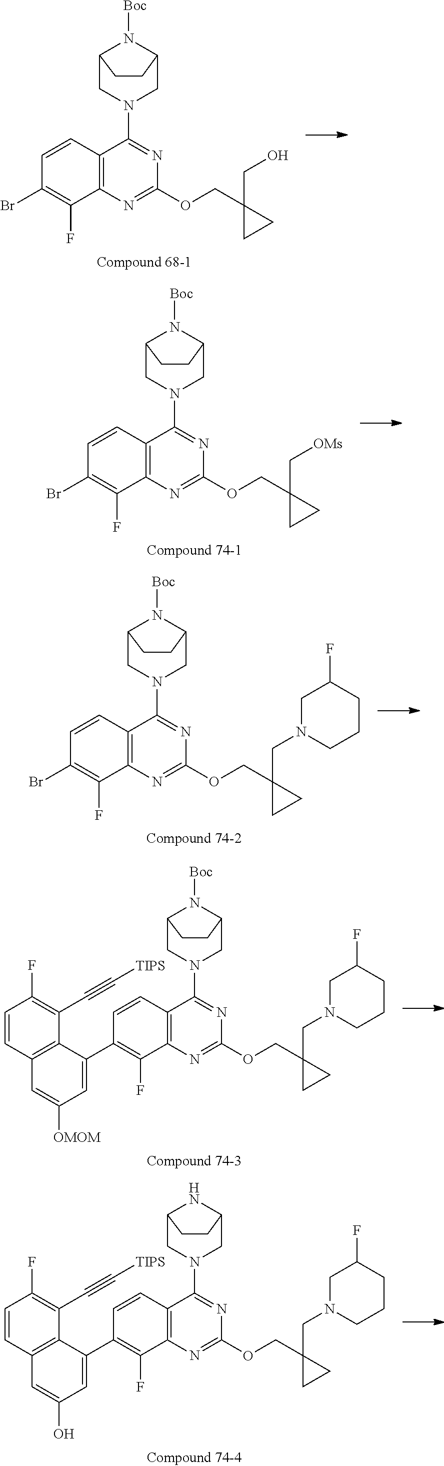

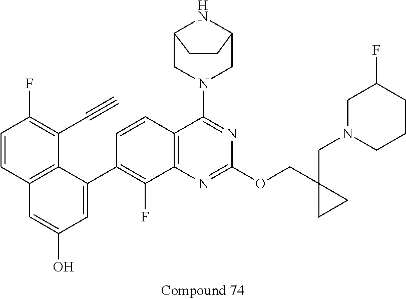 [SMILES: C#Cc1c(F)ccc2cc(O)cc(-c3ccc4c(N5CC6CCC(C5)N6)nc(OCC5(CN6CCCC(F)C6)CC5)nc4c3F)c12]

To a mixture of Compound 68-1 (1.93 g, 3.59 mmol), TEA (1.13 g, 11.17 mmol) in DCM (50 mL) was added MsCl (0.92 g, 8.03 mmol). The reaction mixture was stirred at 0° C. for 1 hrs, diluted with DCM (50 mL) and washed with water (60 mL). The organic layer was dried over anhydrous Na₂SO₄, and concentrated under reduced pressure to afford Compound 74-1 (2.19 g, 2.21 mmol). MS: m/z 615 [M+1]⁺.

A mixture of Compound 74-1 (295 mg, 0.48 mmol), 3-fluoropiperidine hydrochloride (141 mg, 1.37 mmol), TEA (210 mg, 2.08 mmol) and DMF (5 mL) was stirred at 90° C. for 18 hrs, and then diluted with EA (40 mL) and water (30 mL). The organic layer was washed with brine, dried over anhydrous Na₂SO₄, and concentrated under reduced pressure to obtain a residue which was purified with Pre-TLC (Hex:EA=3:2, v/v) to afford Compound 74-2 (143 mg, 0.23 mmol). MS: m/z 622 [M+1]⁺.

A mixture of Compound 74-2 (143 mg, 0.23 mmol), INT C13 (146 mg, 0.28 mmol), Cs₂CO₃ (226 mg, 0.69 mmol), Pd(dppf)Cl₂ (18 mg, 0.025 mmol), 1, 4-dioxane (8 mL) and water (2 mL) was purged with nitrogen, stirred at 100° C. for 20 hrs, and then concentrated under reduced pressure to obtain a residue. The residue was diluted with EA (40 mL) and water (30 mL), and the organic layer was concentrated under reduced pressure to obtain a crude product which was purified with Pre-TLC (Hex:EA=1:1, v/v) to afford Compound 74-3 (61 mg, 0.066 mmol). MS: m/z 928 [M+H]⁺.

A solution of Compound 74-3 (61 mg, 0.065 mmol), HCl/1, 4-dioxane (4 M, 1.5 mL) in CH₃CN (4 mL) was stirred at R.T for 3 hrs, and then concentrated under reduced pressure to afford a crude product containing Compound 74-4 (85 mg, crude). MS: m/z 784 [M+H]⁺.

A mixture of the crude product containing Compound 74-4 (85 mg, crude), CsF (170 mg, 1.12 mmol) and DMF (5 mL) was stirred at R.T for 18 hrs, then diluted with EA (40 mL) and water (30 mL), and the pH of the resulting mixture was adjusted to 8-9 with aq. NaHCO₃(5%). The organic layer was concentrated under reduced pressure to obtain a residue which was purified with Pre-HPLC (C18 column; eluent A: 0.1% TFA in water; eluent B: CH₃CN; Gradient: 10% B to 35% B in 40 min at a flow rate of 40 mL/min; 280 nm) to afford Compound 74 (16.1 mg, 0.026 mmol). MS: m/z 628 [M+H]⁺.

### Example 44

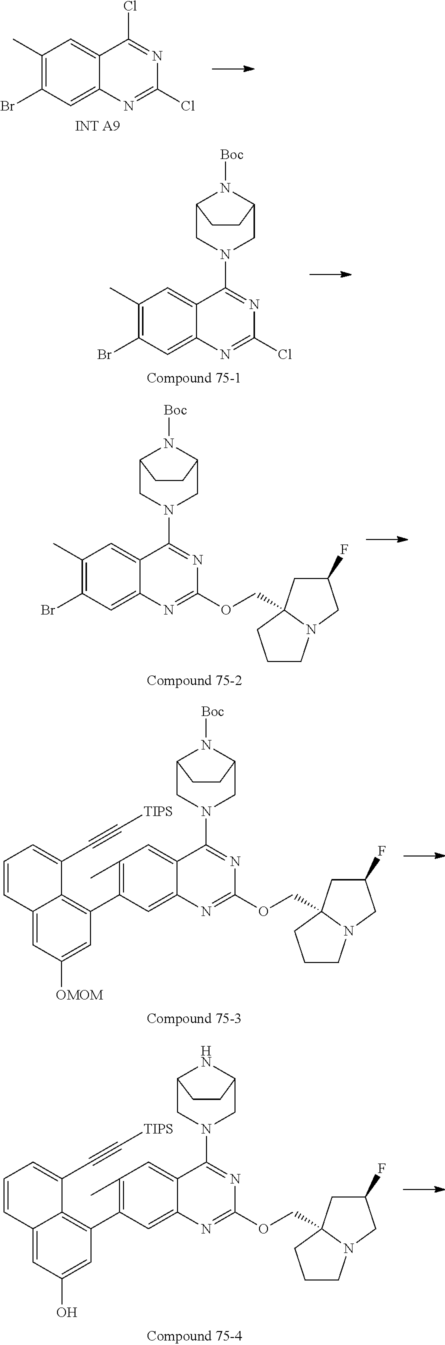

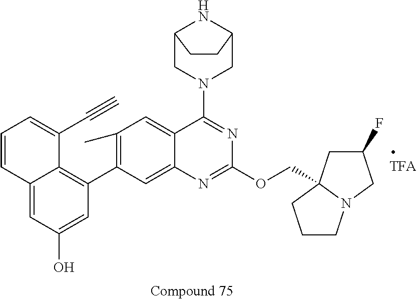 [SMILES: C#Cc1cccc2cc(O)cc(-c3cc4nc(OC[C@@]56CCCN5C[C@H](F)C6)nc(N5CC6CCC(C5)N6)c4cc3C)c12]

INT A9 (400.0 mg, 1.4 mmol, 1.0 eq), 8-boc-3,8-diaza-bicyclo [3.2.1] octane (291 mg, 1.4 mmol, 1.0 eq), TEA (415 mg, 3.9 mmol, 3.0 eq) were added successively into 1,4-dioxane (5 mL). The reaction mixture was stirred for 30 mins at R.T, poured into water (5 mL) after the reaction was completed, and then extracted with EtOAc (5 mL). The organic layer was washed with water (5 mL) and brine (5 mL) successively, dried with anhydrous Na₂SO₄, and concentrated to obtain a residue. The residue was slurried with ether and filtered to afford Compound 75-1 (600.0 mg, yield 93.6%) as a yellow solid. LCMS: 466.8, 468.8 ([M+H]⁺).

Compound 75 was synthesized using Compound 75-1 as starting material according to the synthetic procedure of Compound 67.

### Example 45

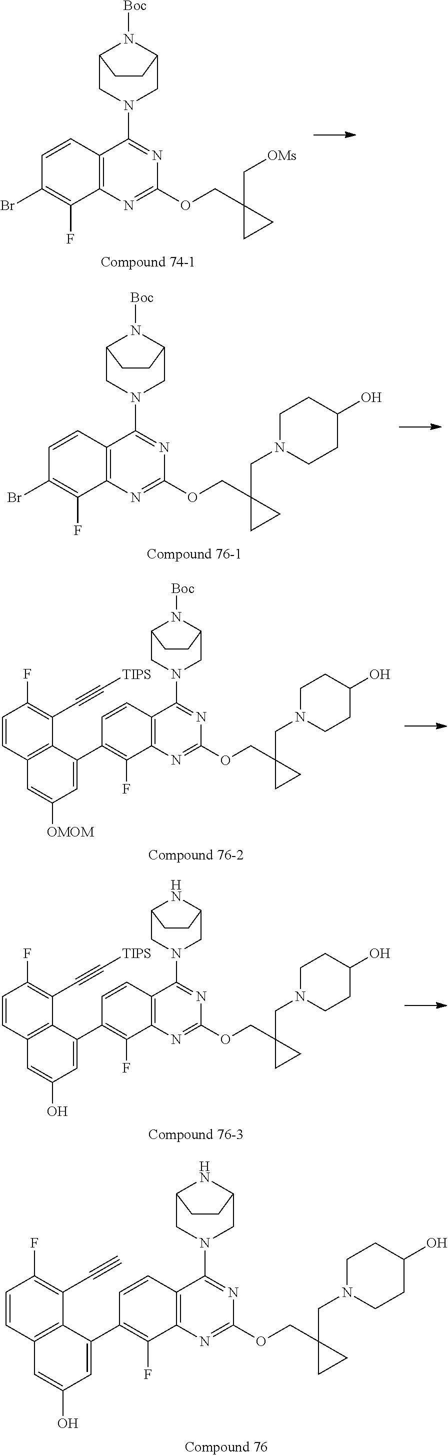

A mixture of Compound 74-1 (339 mg, 0.55 mmol), 4-Hydroxypiperidine (120 mg, 1.19 mmol), TEA (163 mg, 1.61 mmol) and DMF (5 mL) was stirred at 90° C. for 18 hrs, and then diluted with EA (30 mL) and water (30 mL). The organic layer was washed with brine, dried over anhydrous Na₂SO₄ and concentrated under reduced pressure to obtain a residue which was purified with Pre-TLC (DCM:MeOH=10:1, v/v) to afford Compound 76-1 (311 mg, 0.51 mmol). MS: m/z 620 [M+1]⁺.

A mixture of Compound 76-1 (288.6 mg, 0.47 mmol), INT C13 (239 mg, 0.47 mmol), PdCl₂(dppf)₂ (37 mg, 0.051 mmol), Cs₂CO₃ (444 mg, 1.36 mmol), 1, 4-dioxane (8 mL) and water (2 mL) was purged with nitrogen, stirred at 100° C. for 4 hrs, and then concentrated under reduced pressure. The residue was diluted with EA (30 mL) and water (30 mL). The organic layer was concentrated under reduced pressure to obtain a residue which was purified with silica gel chromatography (eluting with Hex:EA from 1:1 to 0:1, v/v) to afford Compound 76-2 (45 mg, 0.049 mmol). MS: m/z 926 [M+H]⁺.

A solution of Compound 76-2 (45 mg, 0.049 mmol), HCl/1, 4-dioxane (4 M, 1.5 mL) in CH₃CN (5 mL) was stirred at R.T for 3 hrs, and then concentrated under reduced pressure to afford a crude product containing Compound 76-3. MS: m/z 782 [M+H]⁺.

To a mixture of the crude product containing Compound 76-3, CsF (186 mg, 1.22 mmol) and DMF (4 mL) was stirred at R.T for 17 hrs, and then filtered. The filtrate was concentrated under reduced pressure to obtain a residue which was purified with Pre-HPLC (C18 column; eluent A: 0.1% TFA in water; eluent B: CH₃CN; Gradient: 15% B to 40% B in 35 min at a flow rate of 60 mL/min; 285 nm) to afford Compound 76 (17.8 mg, 0.021 mmol). MS: m/z 626 [M+H]⁺.

### Example 46

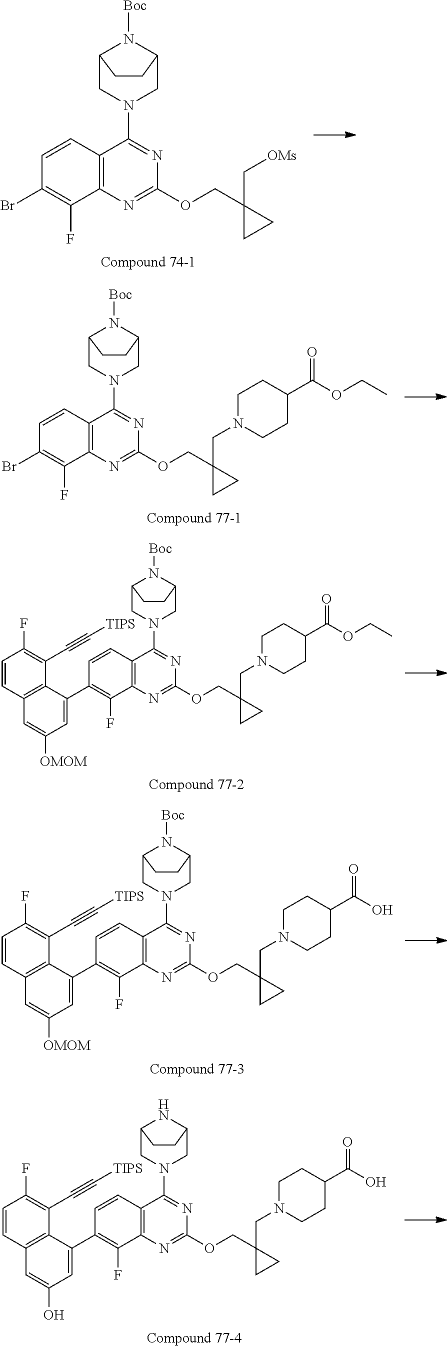

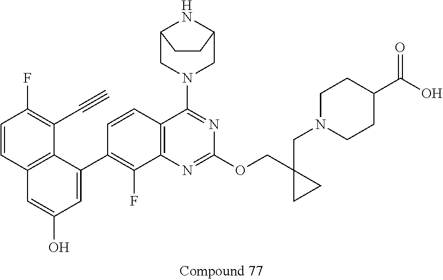 [SMILES: C#Cc1c(F)ccc2cc(O)cc(-c3ccc4c(N5CC6CCC(C5)N6)nc(OCC5(CN6CCC(C(=O)O)CC6)CC5)nc4c3F)c12]

TEA (277 mg, 2.73 mmol) was added to a mixture of Compound 74-1 (407 mg, 0.66 mmol), ethyl 4-piperidinecarboxylate (227 mg, 1.44 mmol) and DMF (5 mL). The reaction mixture was stirred at 90° C. for 20 hrs, and then diluted with EA (45 mL) and water (40 mL). The organic layer was washed with brine, dried over anhydrous Na₂SO₄, and concentrated under reduced pressure to obtain a residue which was purified with Pre-TLC (Hex:EA=1:2, v/v) to afford Compound 77-1 (214 mg, 0.32 mmol). MS: m/z 676 [M+1]⁺.

Pd(dppf)Cl₂ (27 mg, 0.037 mmol) was added to a mixture of Compound 77-1 (214 mg, 0.32 mmol), INT C13 (207 mg, 0.40 mmol), Cs₂CO₃ (309 mg, 0.95 mmol), 1, 4-dioxane (8 mL) and water (2 mL). The reaction mixture was purged with nitrogen, stirred at 100° C. for 6 hrs, and then concentrated under reduced pressure to obtain a residue. The residue was diluted with EA (50 mL), filtered and concentrated under reduced pressure to obtain a crude product which was purified with Pre-TLC (Hex:EA=1:2, v/v) to afford Compound 77-2 (88 mg, 0.090 mmol). MS: m/z 982 [M+H]⁺.

NaOH (19 mg, 0.48 mmol) was added to a solution of Compound 77-2 (80.7 mg, 0.082 mmol) in MeOH (5 mL) and water (1 mL). The reaction mixture was stirred at R.T for 24 hrs, and then concentrated under reduced pressure to afford a crude product containing Compound 77-3 (162 mg). MS: m/z 954 [M+H]⁺.

A solution of the crude product containing Compound 77-3 (162 mg), HCl/1, 4-dioxane (4 M, 2.5 mL) and CH₃CN (7 mL) was stirred at R.T for 2 hrs, and then concentrated under reduced pressure to obtain a residue which was diluted with EA (40 mL) and TEA (0.3 mL). The resulting mixture was filtered and the filtrate was concentrated under reduced pressure to afford a crude product containing Compound 77-4. MS: m/z 810 [M+H]⁺.

A mixture of the crude product containing Compound 77-4, CsF (198 mg, 1.30 mmol) and DMF (4 mL) was stirred at R.T for 19 hrs, and then filtered. The filtrate was concentrated under reduced pressure to obtain a residue which was purified with Pre-HPLC (C18 column; eluent A: 0.1% TFA in water; eluent B: CH₃CN; Gradient: 15% B to 40% B in 30 min at a flow rate of 60 mL/min; 285 nm) to afford Compound 77 (33.7 mg, 0.044 mmol). MS: m/z 654 [M+H]⁺.

### Example 47

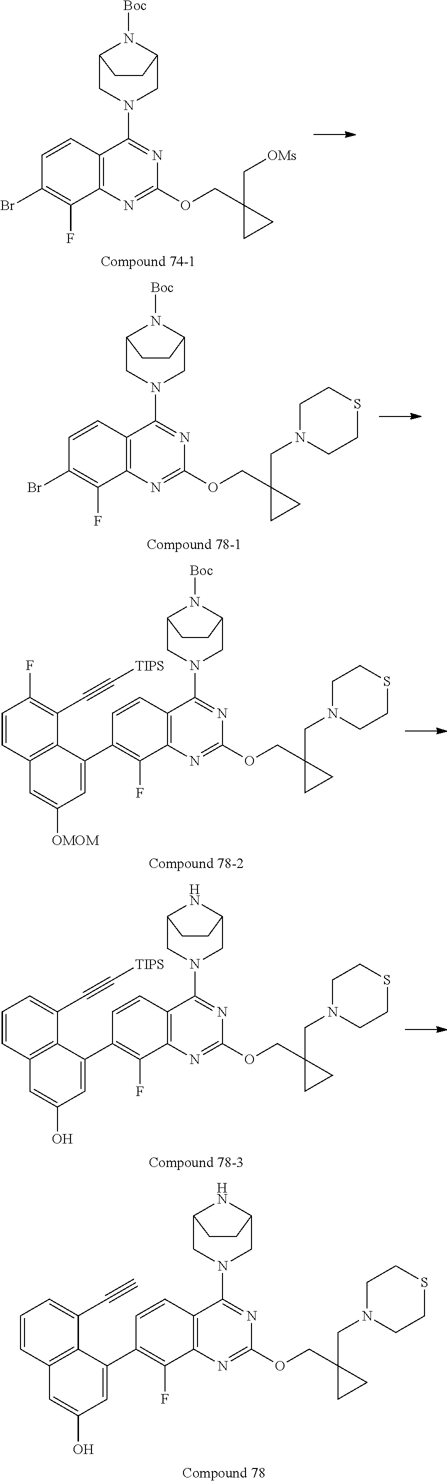

TEA (129 mg, 1.27 mmol) was added to a mixture of Compound 74-1 (250 mg, 0.41 mmol), thiomorpholine (157 mg, 1.52 mmol) and DMF (5 mL). The reaction mixture was stirred at 90° C. for 18 hrs, and then water (30 mL) was added. The resulting mixture was extracted with EA (30 mL), and the organic layer was washed with brine and concentrated under reduced pressure to obtain a residue which was purified with Pre-TLC (Hex:EA=2:1, v/v) to afford Compound 78-1 (170 mg, 0.27 mmol). MS: m/z 622 [M+1]⁺.

CataCXium A Pd G₃ (21 mg, 0.029 mmol) was added to a mixture of Compound 78-1 (170 mg, 0.27 mmol), INT C12 (161 mg, 0.33 mmol), K₃PO₄ (177 mg, 0.83 mmol), THF (8 mL) and water (2 mL). The reaction mixture was purged with nitrogen, stirred at 60° C. for 3 hrs, and then concentrated under reduced pressure to obtain a residue which was diluted with EA (40 mL) and water (30 mL). The organic layer was concentrated under reduced pressure to obtain a crude product which was purified with silica gel chromatography (eluting with Hex:EA=5:1, v/v) to afford Compound 78-2 (195 mg, 0.21 mmol). MS: m/z 910 [M+1]⁺.

A solution of Compound 78-2 (195 mg, 0.21 mmol) and HCl/1, 4-dioxane (4 M, 2 mL) in CH₃CN (5 mL) was stirred at R.T for 2 hrs, and then concentrated under reduced pressure to afford a crude product containing Compound 78-3. MS: m/z 766 [M+H]⁺.

A mixture of the crude product containing Compound 78-3, CsF (296 mg, 1.95 mmol) and DMF (4 mL) was stirred at R.T for 24 hrs, and then filtered. The filtrate was concentrated under reduced pressure to obtain a residue which was purified with Pre-HPLC (C18 column; eluent A: 0.1% TFA in water; eluent B: CH₃CN; Gradient: 10% B to 40% B in 40 min at a flow rate of 60 mL/min; 234 nm) to afford Compound 78 (101.6 mg, 1.21 mmol). MS: m/z 610 [M+H]⁺.

### Example 48

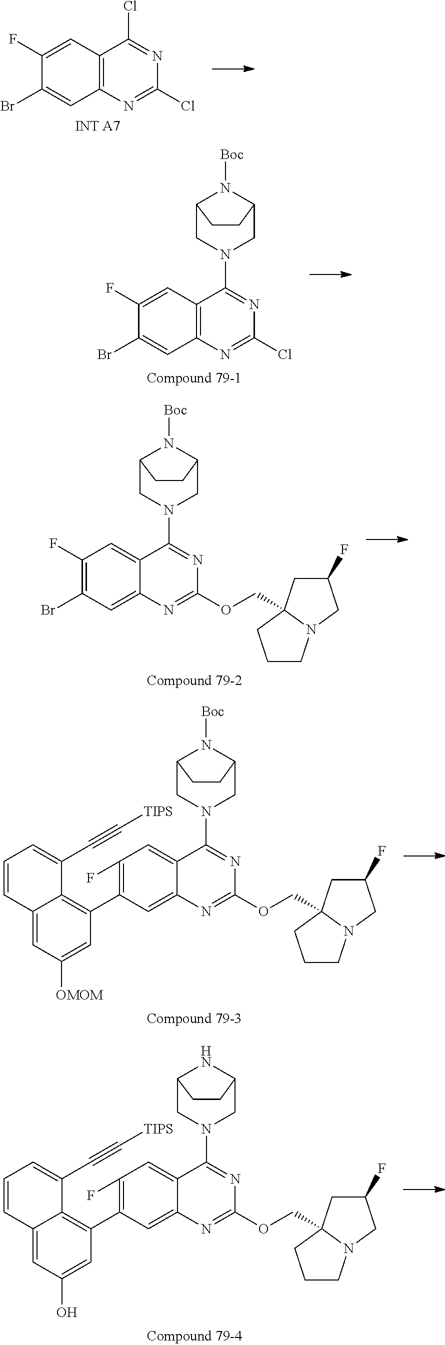

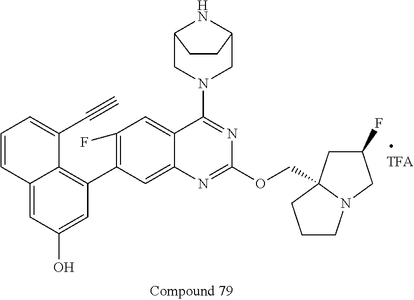 [SMILES: C#Cc1cccc2cc(O)cc(-c3cc4nc(OC[C@@]56CCCN5C[C@H](F)C6)nc(N5CC6CCC(C5)N6)c4cc3F)c12]

Tert-butyl (1R, 5S)-3,8-diazabicyclo[3.2.1]octane-8-carboxylate (716.4 mg, 3.38 mmol, 1.0 eq) and TEA (1.03 g, 10.14 mmol, 3.0 eq) were added to a solution of the crude product containing INT A7 (1.0 g, 3.38 mmol, 1.0 eq) in dioxane (10 mL). The reaction mixture was stirred at R.T for 1.5 hrs, diluted with water (30 mL), and extracted with DCM (30 mL×3). The organic layers were combined, washed with saturated aq. NaCl (30 mL), and concentrated to obtain a residue which was purified by silica gel chromatography (petroleum ether/ethyl acetate=25:1 to 8:1) to afford Compound 79-1 (1.1 g, 69.2%) as a yellow solid. ¹H NMR (300 MHz, DMSO-d₆): δ 8.19 (d, J=6.9 Hz, 1H), 8.07 (d, J=9.9 Hz, 1H), 4.44-4.30 (m, 4H), 3.67-3.63 (m, 2H), 1.86-1.84 (m, 2H), 1.70-1.67 (m, 2H), 1.52 (s, 9H). LCMS: 471.0 ([M+H]⁺).

Compound 79 was synthesized using Compound 79-1 as starting material according to the synthetic procedure of Compound 67.

### Example 49

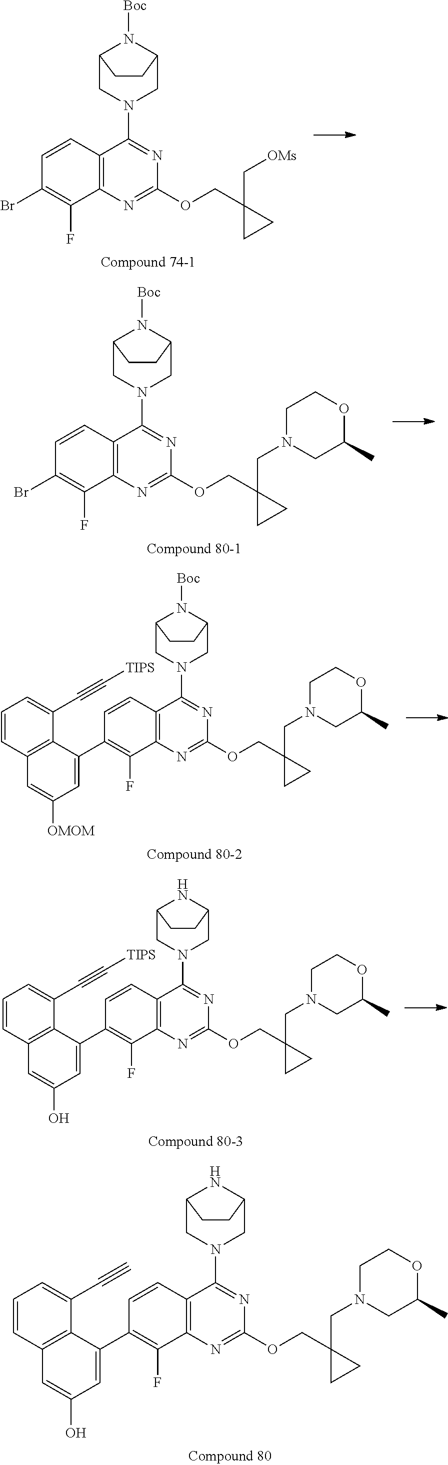

TEA (246 mg, 2.43 mmol) was added to a solution of (S)-2-methylmorpholine hydrochloride (141 mg, 1.02 mmol) in DMF (5 mL). The mixture was stirred at R.T for 1.5 hrs, and then Compound 74-1 (297 mg, 0.48 mmol) was added. The reaction mixture was stirred at 90° C. for 16 hrs, diluted with water (30 mL) and extracted with EA (40 mL). The organic layer was washed with brine, dried over anhydrous Na₂SO₄, and concentrated under reduced pressure to obtain a residue which was purified with Pre-TLC (Hex:EA=2:1, v/v) to afford Compound 80-1 (141 mg, 0.23 mmol). MS: m/z 620 [M+1]⁺.

CataCXium A Pd G₃ (17 mg, 0.023 mmol) was added to a mixture of Compound 80-1 (141 mg, 0.23 mmol), INT C12 (142 mg, 0.29 mmol), K₃PO₄ (267 mg, 1.26 mmol), THF (8 mL) and water (2 mL). The reaction mixture was purged with nitrogen, stirred at 60° C. for 2 hrs, and then concentrated under reduced pressure to obtain a residue which was diluted with EA (35 mL) and water (30 mL). The organic layer was separated, and concentrated under reduced pressure to obtain a crude product which was purified with silica gel chromatography (eluting with Hex:EA=4:1, v/v) to afford Compound 80-2 (192 mg, 0.21 mmol). MS: m/z 908 [M+1]⁺.

A solution of Compound 80-2 (192 mg, 0.21 mmol) and HCl/1,4-dioxane (4 M, 2 mL) in CH₃CN (5 mL) was stirred at R.T for 2 hrs, and then concentrated under reduced pressure to obtain a residue which was diluted with EA (40 mL) and water (30 mL). The pH of the resulting mixture was adjusted to 8-9 with aq. NaHCO₃ (5%). The organic layer was washed with brine, dried over anhydrous Na₂SO₄, and concentrated under reduced pressure to afford Compound 80-3 (156 mg, 0.20 mmol). MS: m/z 764 [M+H]⁺.

A mixture of Compound 80-3 (156 mg, 0.20 mmol), CsF (349 mg, 2.30 mmol) and DMF (5 mL) was stirred at R.T for 18 hrs, and then diluted with EA (40 mL) and water (30 mL). The pH of the resulting mixture was adjusted to 8-9 with aq. NaHCO₃ (5%). The organic layer was separated and concentrated under reduced pressure to obtain a residue which was purified with Pre-HPLC (C18 column; eluent A: 0.1% TFA in water; eluent B: CH₃CN; Gradient: 15% B to 35% B in 35 min at a flow rate of 60 mL/min; 234 nm) to afford Compound 80 (123.9 mg, 0.15 mmol). MS: m/z 608 [M+H]⁺.

### Example 50

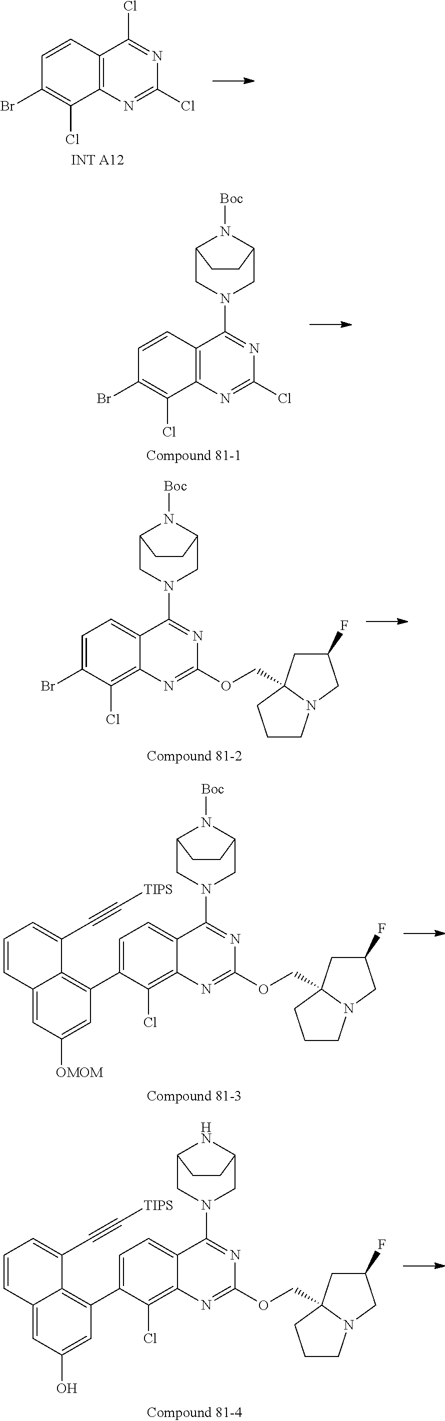

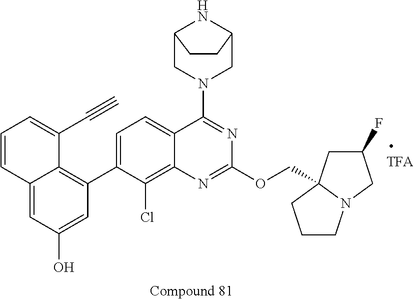 [SMILES: C#Cc1cccc2cc(O)cc(-c3ccc4c(N5CC6CCC(C5)N6)nc(OC[C@@]56CCCN5C[C@H](F)C6)nc4c3Cl)c12]

Compound 81 was obtained following the procedure of Compound 67 with INT A12 as start material.

### Example 51

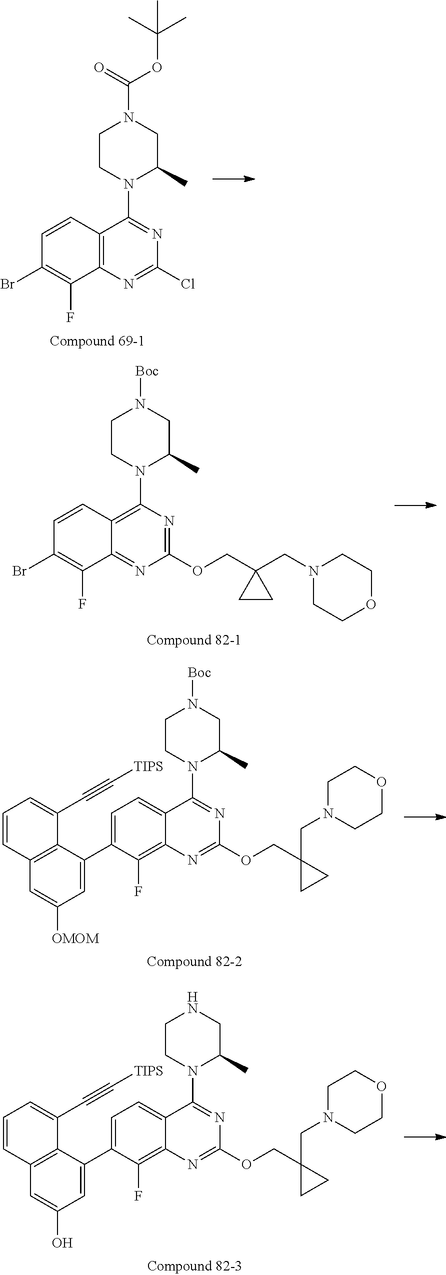

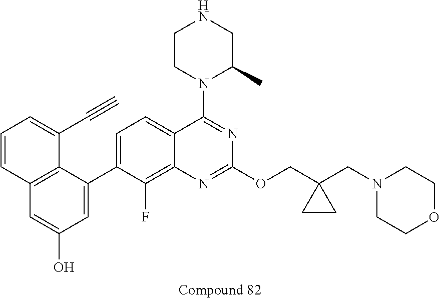 [SMILES: C#Cc1cccc2cc(O)cc(-c3ccc4c(N5CCNC[C@H]5C)nc(OCC5(CN6CCOCC6)CC5)nc4c3F)c12]

Compound 82 was obtained following the procedure of Compound 69 with Compound 69-1 as start material.

### Example 52

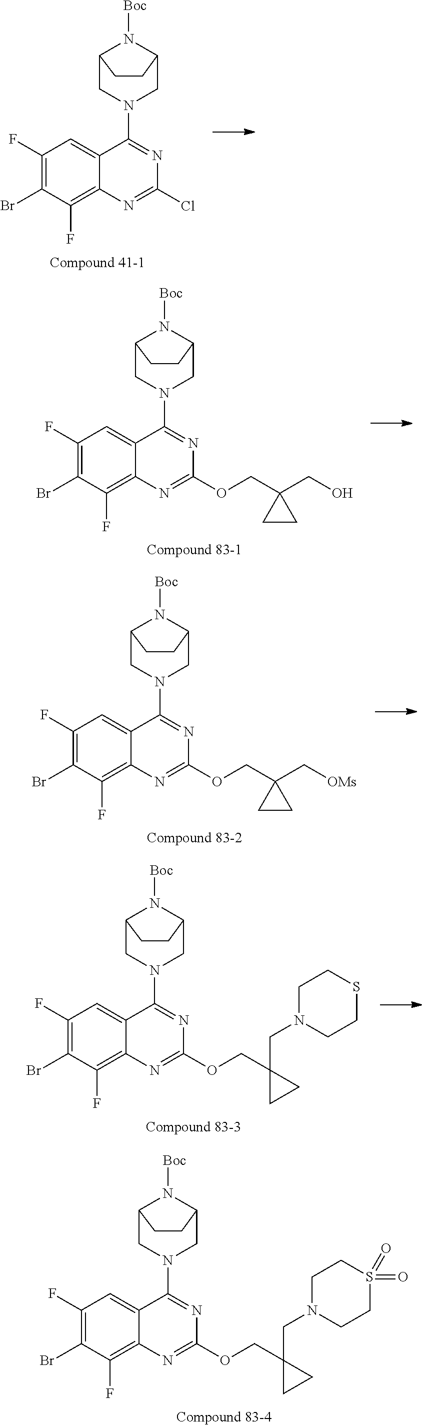 [SMILES: CC(C)(C)OC(=O)N1C2CCC1CN(c1nc(Cl)nc3c(F)c(Br)c(F)cc13)C2 ; CC(C)(C)OC(=O)N1C2CCC1CN(c1nc(OCC3(CO)CC3)nc3c(F)c(Br)c(F)cc13)C2 ; CC(C)(C)OC(=O)N1C2CCC1CN(c1nc(OCC3(COS(C)(=O)=O)CC3)nc3c(F)c(Br)c(F)cc13)C2 ; CC(C)(C)OC(=O)N1C2CCC1CN(c1nc(OCC3(CN4CCSCC4)CC3)nc3c(F)c(Br)c(F)cc13)C2 ; CC(C)(C)OC(=O)N1C2CCC1CN(c1nc(OCC3(CN4CCS(=O)(=O)CC4)CC3)nc3c(F)c(Br)c(F)cc13)C2]

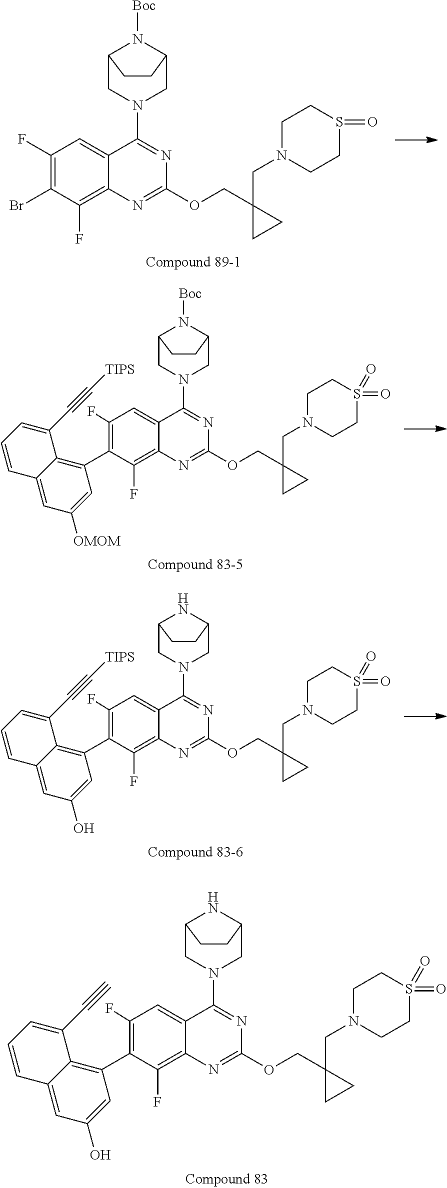

Compound 41-1 (13.5 g, 27.6 mmol, 1.0 eq.), cyclopropane-1,1-diyldimethanol (8.45 g, 82.6 mmol, 3.0 eq.), triethylenediamine (3.1 g, 27.6 mmol, 1.0 eq.), Cs₂CO₃ (27 g, 82.6 mmol, 3.0 eq.) were added successively into a mixed solution of THF and DMF (V_{THF}/V_{DMF}=1:1, 200 mL). The mixture was stirred at room temperature for 5 hrs, poured into water (200 mL) after the reaction was completed, and then extracted with EtOAc (200 mL). The organic layer was washed with brine (50 mL), dried with Na₂SO₄, and then concentrated to obtain a residue which was purified with silica gel chromatography (eluting with petroleum ether/EtOAc=5:1) to afford Compound 83-1 (9.75 g, yield 63.6%) as a white solid. LCMS: 555 ([M+H]⁺).

To a mixture of Compound 83-1(1.20 g, 2.16 mmol), TEA (0.69 g, 6.81 mmol) in DCM (25 mL) was added MsCl (0.62 g, 5.41 mmol). The reaction mixture was stirred at 0° C. for 0.5 hrs, diluted with DCM (10 mL) and washed with water (30 mL). The organic layer was dried over anhydrous Na₂SO₄, and concentrated under reduced pressure to afford Compound 83-2 (1.53 g, 2.42 mmol). MS: m/z 633 [M+1]⁺.

TEA (116 mg, 1.15 mmol) was added to a solution of Compound 83-2 (325 mg, 0.51 mmol) and thiomorpholine (141 mg, 1.37 mmol) in DMF (5 mL). The mixture was stirred at 90° C. for 24 hrs, and then diluted with EA (40 mL) and water (40 mL). The organic layer was separated, washed with brine, dried over anhydrous Na₂SO₄, and concentrated under reduced pressure to obtain a residue which was purified with Pre-TLC (Hex:EA=2:1, v/v) to afford Compound 83-3 (232 mg, 0.36 mmol). MS: m/z 640 [M+1]⁺.

M-CPBA (146 mg, 2.24 mmol) was added to a solution of Compound 83-3 (222 mg, 0.35 mmol) in DCM (15 mL) at 0° C. The reaction mixture was stirred at 0° C. for 30 mins, and then concentrated under reduced pressure to obtain a residue which was diluted with EA (40 mL) and water (30 mL). The pH of the resulting mixture was adjusted to 9-10 with NaHCO₃. The organic layer was separated and concentrated under reduced pressure to obtain a crude product which was purified with Pre-TLC (DCM:MeOH=20:1, v/v) to afford Compound 83-4 (208 mg, 0.31 mmol). MS: m/z 672 [M+1]⁺.

CataCXium A Pd G₃ (22 mg, 0.030 mmol) was added to a mixture of Compound 83-4 (208 mg, 0.31 mmol), INT C12 (177 mg, 0.36 mmol), Cs₂CO₃ (307 mg, 0.94 mmol), toluene (8 mL) and water (2 mL). The reaction mixture was purged with nitrogen, stirred at 100° C. for 17 hrs, and concentrated under reduced pressure to obtain a residue which was diluted with EA (40 mL) and water (30 mL). The organic layer was separated, washed with brine, dried over anhydrous Na₂SO₄, filtered and concentrated under reduced pressure to obtain a crude product which was purified with Pre-TLC (DCM:MeOH=10:1, v/v) to afford Compound 83-5 (64 mg, 0.067 mmol). MS: m/z 960 [M+H]⁺.

A solution of Compound 83-5 (64 mg, 0.067 mmol) and HCl/1,4-dioxane (4 M, 2 mL) in CH₃CN (5 mL) was stirred at R.T for 1 h, and then concentrated under reduced pressure to afford a crude product containing Compound 83-6. MS: m/z 816 [M+H]⁺.

A mixture of the crude product containing Compound 83-6, CsF (339 mg, 2.23 mmol) and DMF (6 mL) was stirred at R.T for 16 hrs, and then filtered. The filtrate was concentrated under reduced pressure to obtain a residue which was purified with Pre-HPLC (C18 column; eluent A: 0.1% TFA in water; eluent B: CH₃CN; Gradient: 15% B to 45% B in 40 min at a flow rate of 60 mL/min; 240 nm) to afford Compound 83 (14 mg, 0.016 mmol). MS: m/z 660 [M+H]⁺.

### Example 53

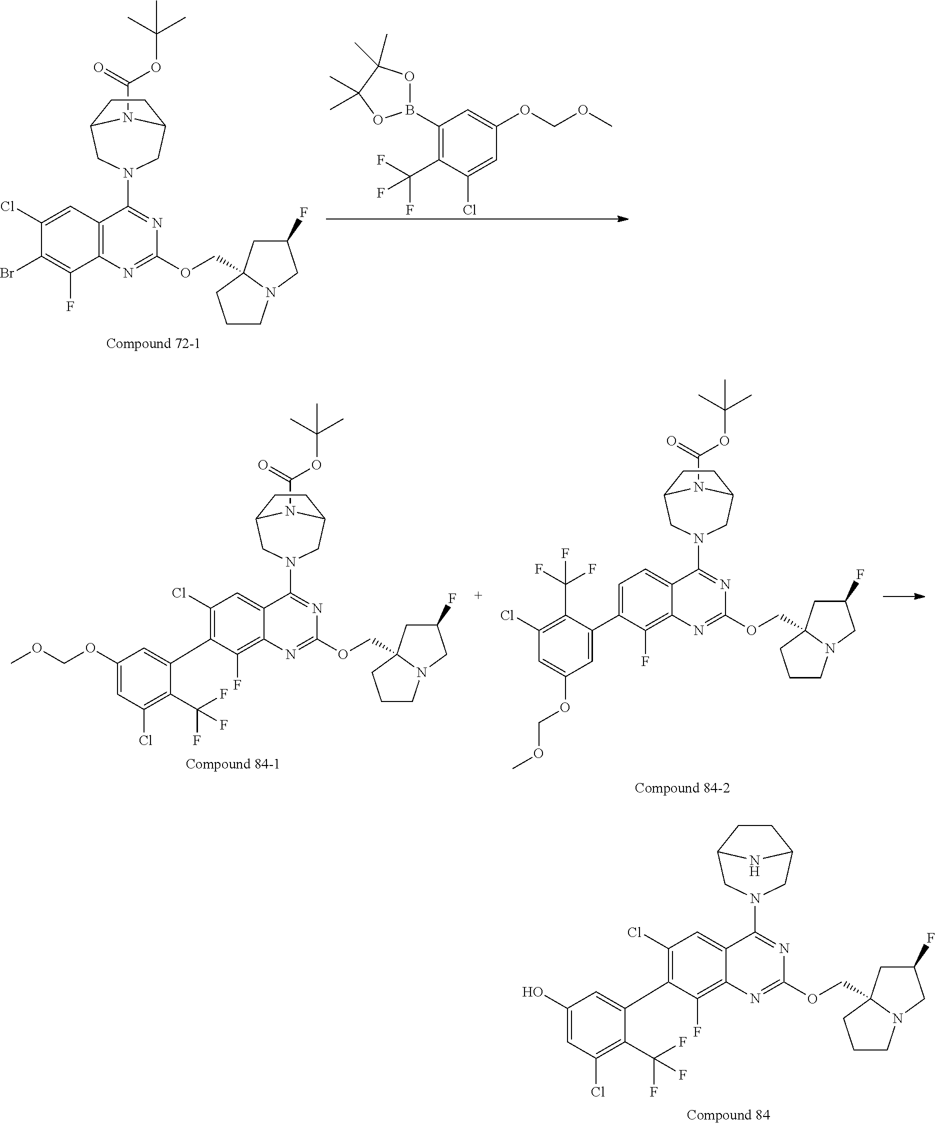

Compound 84 was obtained following the procedure of Compound 69 with Compound 72-1 as start material.

### Example 54

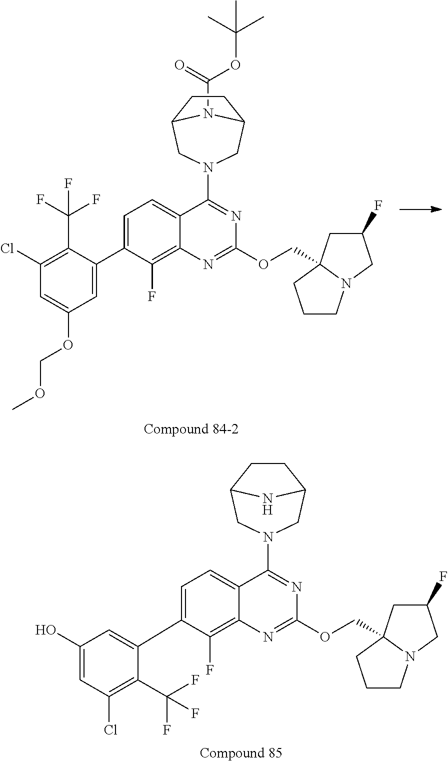 [SMILES: COCOc1cc(Cl)c(C(F)(F)F)c(-c2ccc3c(N4CC5CCC(C4)N5C(=O)OC(C)(C)C)nc(OC[C@@]45CCCN4C[C@H](F)C5)nc3c2F)c1 ; Oc1cc(Cl)c(C(F)(F)F)c(-c2ccc3c(N4CC5CCC(C4)N5)nc(OC[C@@]45CCCN4C[C@H](F)C5)nc3c2F)c1]

Compound 85 was obtained following the procedure of Compound 69 with Compound 84-2 as start material.

### Example 55

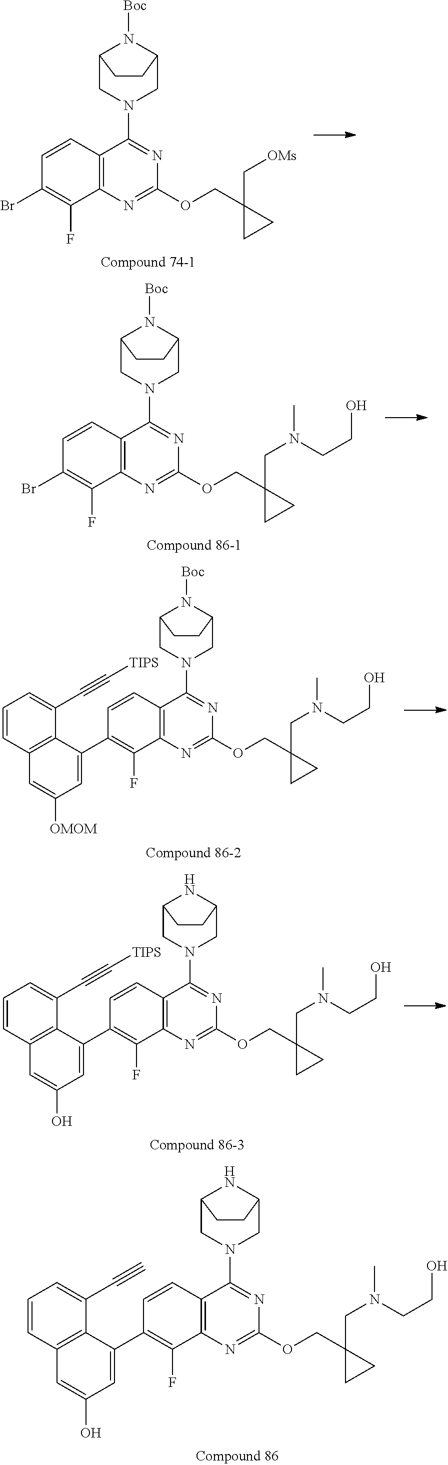

Compound 86 was obtained following the procedure of Compound 74 with Compound 74-1 as start material.

### Example 56

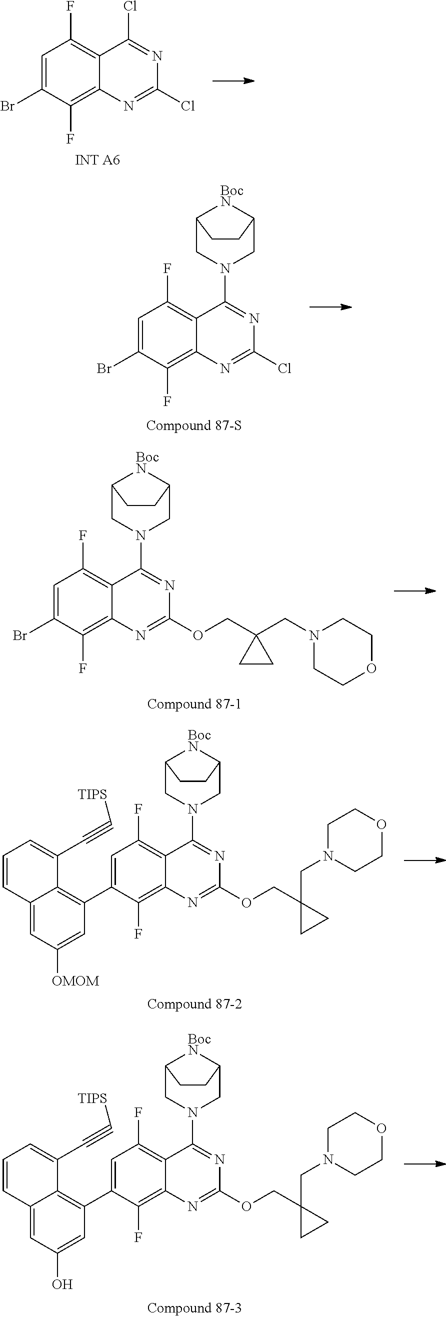

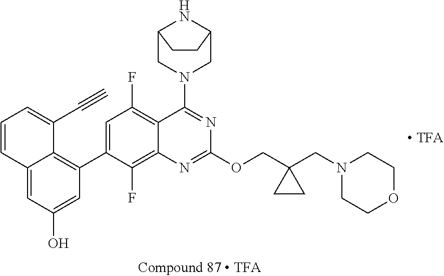 [SMILES: C#Cc1cccc2cc(O)cc(-c3cc(F)c4c(N5CC6CCC(C5)N6)nc(OCC5(CN6CCOCC6)CC5)nc4c3F)c12]

Tert-butyl (1R,5S)-3,8-diazabicyclo[3.2.1]octane-8-carboxylate (1.19 g, 5.60 mmol) was added to a solution of INT A6 (1.87 g, 5.95 mmol) and N,N-diisopropylethylamine (1.4 mL) in DCM (15 mL) at R.T. The reaction was stirred for 2 hrs, poured into water (50 mL) and extracted with DCM (40 mL×2). The organic layers were combined and washed with brine (10 mL×2), dried over Na2SO4 and concentrated under reduced pressure to afford Compound 87-S (1.83 g, 3.73 mmol, 62.7% yield). LCMS: 489 [M+H]+.

A mixture of Compound 87-S (1.83 g, 3.73 mmol), potassium fluoride (78 mg, 1.34 mmol) and INT B2 (156 mg, 911.02 μmol) and DMSO (5 mL) was stirred at 120° C. for 16 hrs under nitrogen atmosphere, cooled to R.T, and then water (30 mL) and EA (50 mL) were added. The organic layer was separated, dried over Na₂SO₄ and concentrated under reduced pressure to obtain a residue which was purified with Prep-TLC (MeOH:DCM=1:20, v/v) to afford Compound 87-1 (135 mg, 216.16 μmol, 52.6% yield). LCMS: 624 [M+H]⁺.

A mixture of Compound 87-1 (135 mg, 216.16 μmol), INT C12 (106 mg, 214.33 μmol), cataCXium A Pd G₃ (14 mg, 19.22 mol), potassium phosphate (109 mg, 513.50 μmol), toluene (8 mL) and water (2.0 mL) was stirred at 100° C. for 2.5 hrs under nitrogen atmosphere. The resulting mixture was diluted with EA (50 mL) and washed with water (2×30 mL). The organic layer was dried over Na₂SO₄ and concentrated under reduced pressure to obtain a residue which was purified with Pre-TLC (MeOH:DCM=1:20, v/v) to afford Compound 87-2 (141 mg, 154.57 μmol, 91.9% yield). MS m/z: 912 [M+H]⁺.

A solution of Compound 87-2 (141 mg, 154.57 μmol), HCl/1,4-dioxane (4 M in 1,4-dioxane, 1.5 mL) in CH₃CN (6 mL) was stirred at R.T for 1 h, and then concentrated under reduced pressure to obtain a residue containing Compound 87-3 which was used directly for next step without purification. MS m/z: 768 [M+H]⁺.

A mixture of the residue containing Compound 87-3, CsF (376 mg, 2.47 mmol) and DMF (5 mL) was stirred at 40° C. for 16 hrs, and then concentrated under reduced pressure to obtain a residue which was purified with Pre-HPLC (Daisogel-C18 column, 50*250 mm, 10 μm; eluent A: 0.1% TFA in water; eluent B: CH₃CN; Gradient: 15% B to 45% B in 35 min at a flow rate of 60 mL/min; 290 nm) to afford the TFA salt of Compound 87 (80.4 mg, 110.78 μmol, 71.6% yield). MS m/z: 612 [M+H]⁺.

### Example 57

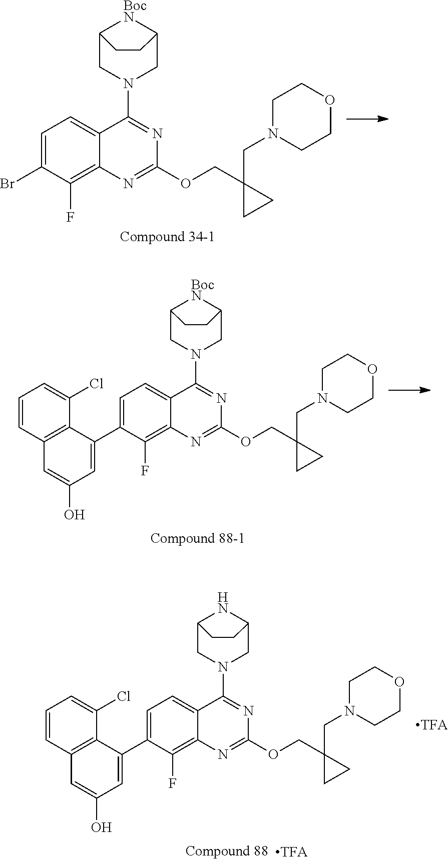 [SMILES: CC(C)(C)OC(=O)N1C2CCC1CN(c1nc(OCC3(CN4CCOCC4)CC3)nc3c(F)c(Br)ccc13)C2 ; CC(C)(C)OC(=O)N1C2CCC1CN(c1nc(OCC3(CN4CCOCC4)CC3)nc3c(F)c(-c4cc(O)cc5cccc(Cl)c45)ccc13)C2 ; Oc1cc(-c2ccc3c(N4CC5CCC(C4)N5)nc(OCC4(CN5CCOCC5)CC4)nc3c2F)c2c(Cl)cccc2c1]

A mixture of Compound 34-1 (58 mg, 95.62 μmol), INT C22 (39 mg, 128.04 μmol), Pd(dppf)Cl₂ (12 mg, 14.76 μmol), Cs₂CO₃ (89 mg, 273.15 μmol), 1,4-dioxane (6 mL) and water (2 mL) was stirred at 100° C. for 2.5 hrs under nitrogen atmosphere, cooled to R.T, diluted with EA (50 mL) and then washed with water (2×30 mL). The organic layer was dried over Na₂SO₄ and concentrated under reduced pressure to obtain a residue which was purified with Pre-TLC (MeOH:DCM=1:13, v/v) to afford Compound 88-1 (72 mg, 102.23 μmol, 106.9% yield). MS m/z: 704 [M+H]⁺.

TFA (1.5 mL) was added to a solution of Compound 88-1 (72 mg, 102.23 μmol) in DCM (6.0 mL). The reaction mixture was stirred at R.T for 1 h, and then concentrated under reduced pressure to obtain a residue which was purified with Pre-HPLC (Daisogel-C18 column, 50*250 mm, 10 μm; eluent A: 0.1% TFA in water, eluent B: CH₃CN; Gradient: 15% B to 35% B in 35 min at a flow rate of 200 mL/min; 231 nm) to afford the TFA salt of Compound 88 (47.7 mg, 66.42 μmol, yield 64.9%). MS m/z: 604 [M+H]⁺.

### Example 58

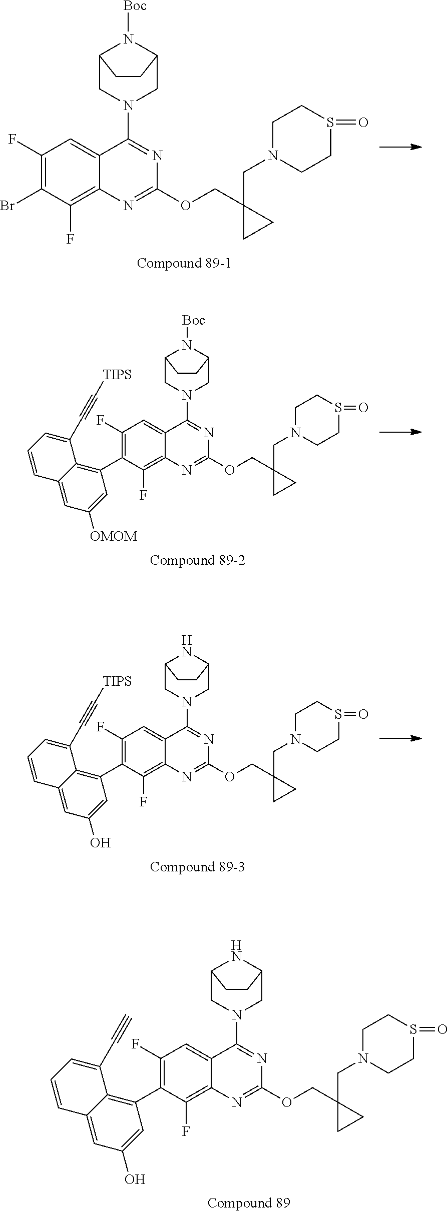

Compound 89 was obtained following the procedure of Compound 83 with Compound 89-1 as start material.

### Example 59

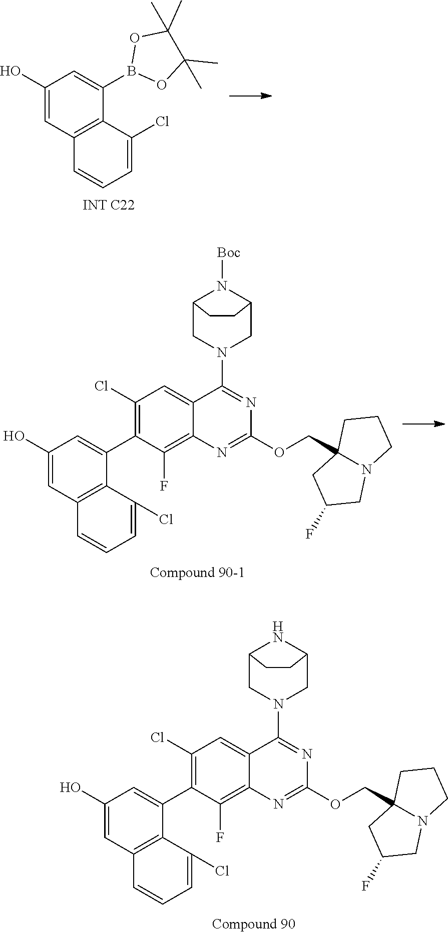

Compound 90 was obtained following the procedure of Compound 35 with Compound 72-1 and INT C22 as start material.

### Example 60

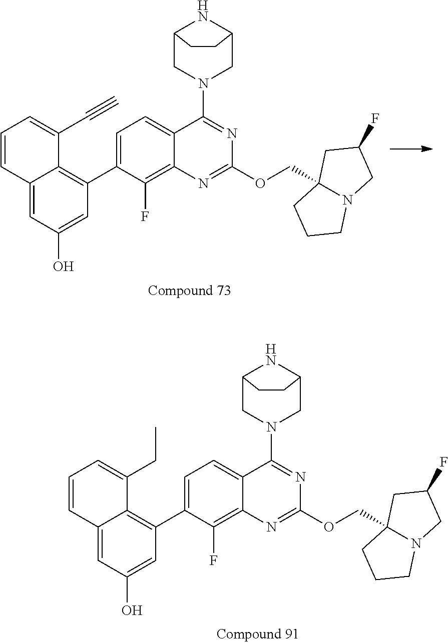 [SMILES: C#Cc1cccc2cc(O)cc(-c3ccc4c(N5CC6CCC(C5)N6)nc(OC[C@@]56CCCN5C[C@H](F)C6)nc4c3F)c12 ; CCc1cccc2cc(O)cc(-c3ccc4c(N5CC6CCC(C5)N6)nc(OC[C@@]56CCCN5C[C@H](F)C6)nc4c3F)c12]

Compound 91 was obtained following the procedure of Compound 36 with Compound 73 as start material.

### Example 61

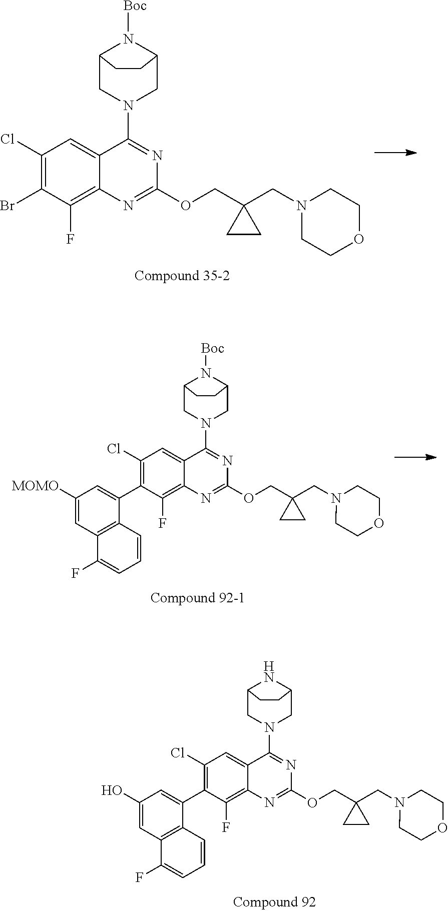

Compound 92 was obtained following the procedure of Compound 50 with Compound 35-2 as start material.

### Example 62

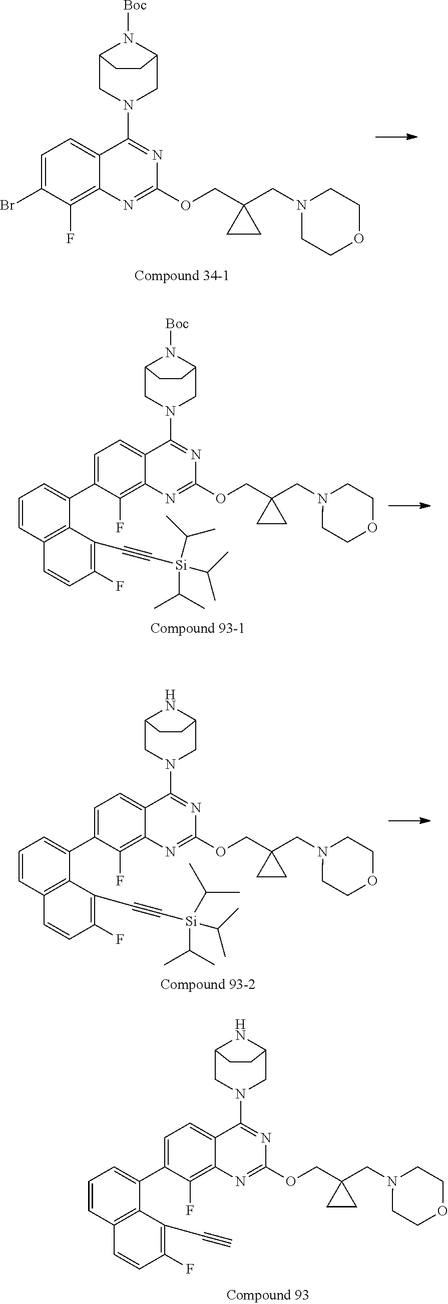 [SMILES: CC(C)(C)OC(=O)N1C2CCC1CN(c1nc(OCC3(CN4CCOCC4)CC3)nc3c(F)c(Br)ccc13)C2 ; CC(C)[Si](C#Cc1c(F)ccc2cccc(-c3ccc4c(N5CC6CCC(C5)N6C(=O)OC(C)(C)C)nc(OCC5(CN6CCOCC6)CC5)nc4c3F)c12)(C(C)C)C(C)C ; CC(C)[Si](C#Cc1c(F)ccc2cccc(-c3ccc4c(N5CC6CCC(C5)N6)nc(OCC5(CN6CCOCC6)CC5)nc4c3F)c12)(C(C)C)C(C)C ; C#Cc1c(F)ccc2cccc(-c3ccc4c(N5CC6CCC(C5)N6)nc(OCC5(CN6CCOCC6)CC5)nc4c3F)c12]

Compound 93 was obtained following the procedure of Compound 34 with Compound 34-1 and INT C26 as start materials.

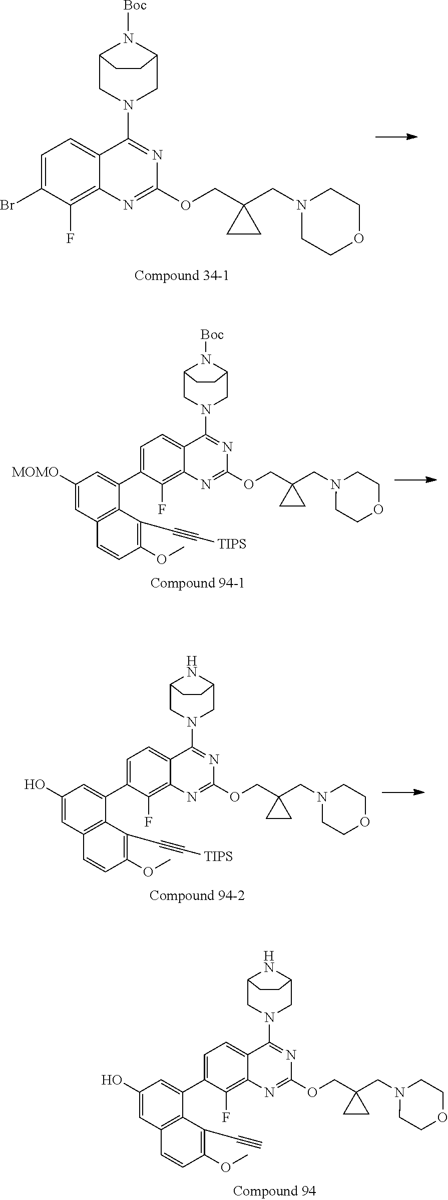

### Example 63

Compound 94 was obtained following the procedure of Compound 48 with Compound 34-1 and INT C25 as start materials.

### Example 64

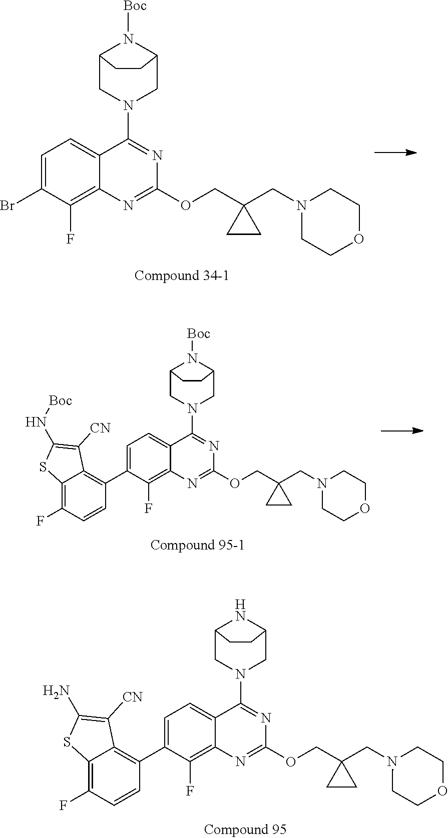 [SMILES: CC(C)(C)OC(=O)N1C2CCC1CN(c1nc(OCC3(CN4CCOCC4)CC3)nc3c(F)c(Br)ccc13)C2 ; CC(C)(C)OC(=O)Nc1sc2c(F)ccc(-c3ccc4c(N5CC6CCC(C5)N6C(=O)OC(C)(C)C)nc(OCC5(CN6CCOCC6)CC5)nc4c3F)c2c1C#N ; N#Cc1c(N)sc2c(F)ccc(-c3ccc4c(N5CC6CCC(C5)N6)nc(OCC5(CN6CCOCC6)CC5)nc4c3F)c12]

Compound 95 was obtained following the procedure of Compound 48 with Compound 34-1 and INT C27 as start materials.

### Example 65

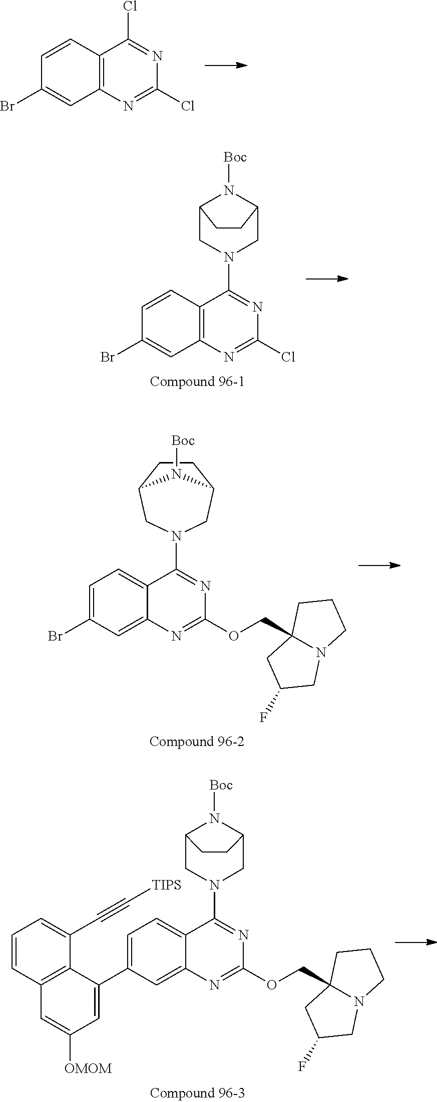

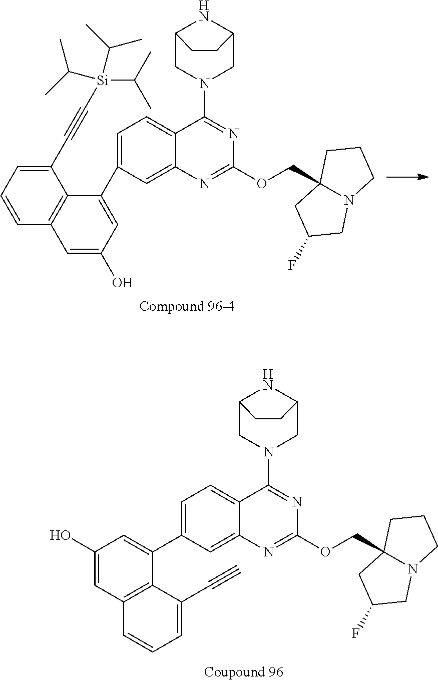 [SMILES: CC(C)[Si](C#Cc1cccc2cc(O)cc(-c3ccc4c(N5CC6CCC(C5)N6)nc(OC[C@@]56CCCN5C[C@H](F)C6)nc4c3)c12)(C(C)C)C(C)C ; C#Cc1cccc2cc(O)cc(-c3ccc4c(N5CC6CCC(C5)N6)nc(OC[C@@]56CCCN5C[C@H](F)C6)nc4c3)c12]

Compound 96 was obtained following the procedure of Compound 36 with 7-bromo-2,4-dichloroquinazoline as start material.

### Example 66

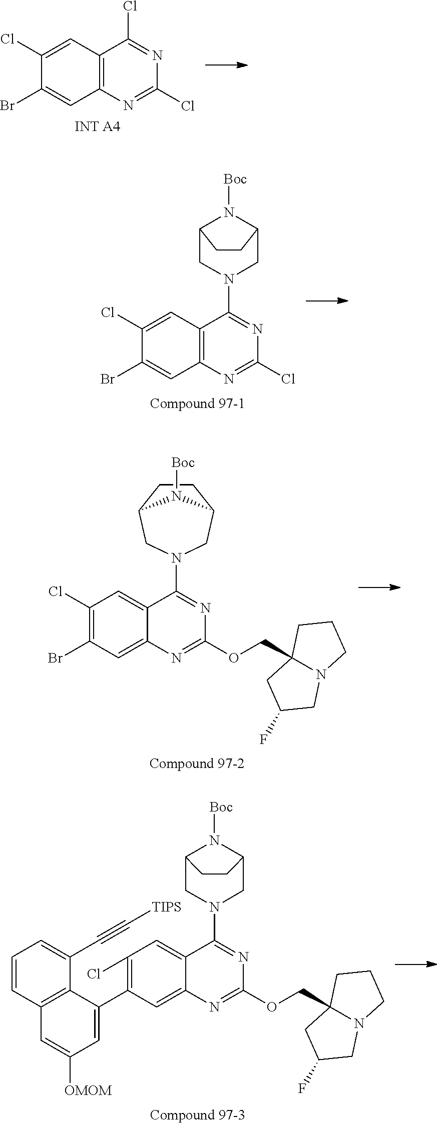

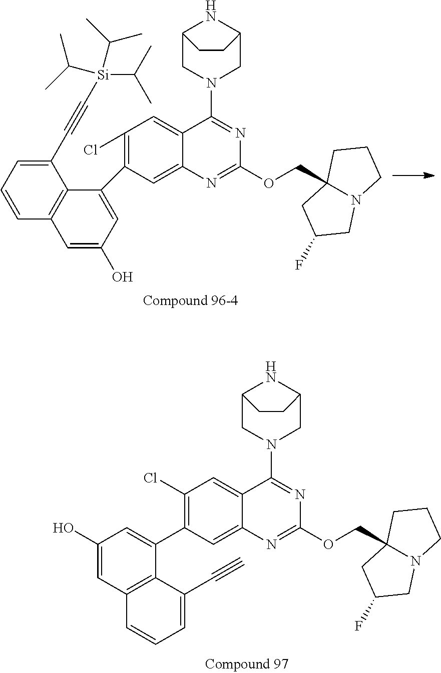 [SMILES: CC(C)[Si](C#Cc1cccc2cc(O)cc(-c3cc4nc(OC[C@@]56CCCN5C[C@H](F)C6)nc(N5CC6CCC(C5)N6)c4cc3Cl)c12)(C(C)C)C(C)C ; C#Cc1cccc2cc(O)cc(-c3cc4nc(OC[C@@]56CCCN5C[C@H](F)C6)nc(N5CC6CCC(C5)N6)c4cc3Cl)c12]

Compound 97 was obtained following the procedure of Compound 36 with INT A4 as start material.

### Example 67

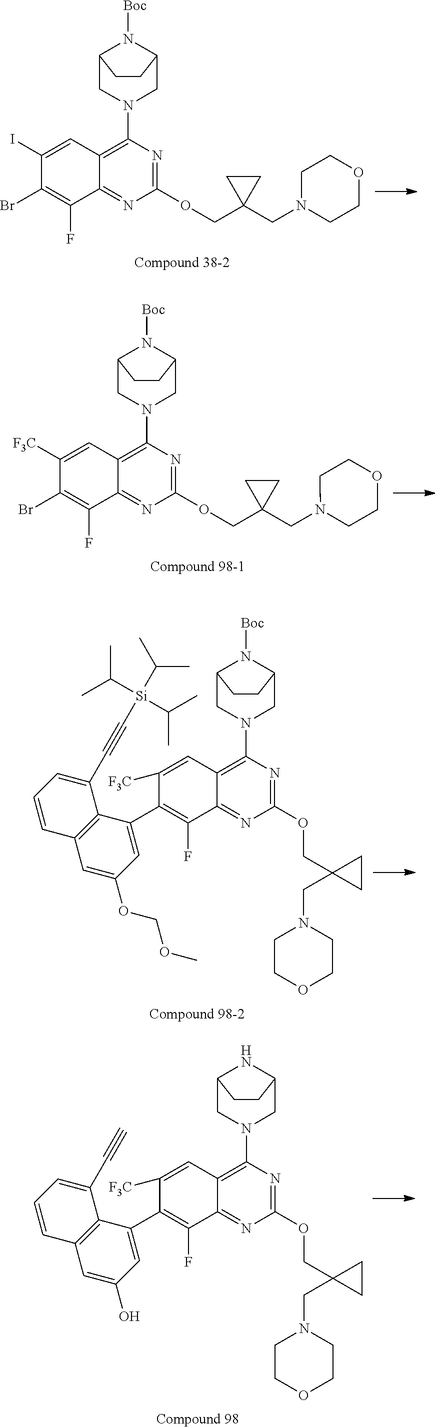 [SMILES: CC(C)(C)OC(=O)N1C2CCC1CN(c1nc(OCC3(CN4CCOCC4)CC3)nc3c(F)c(Br)c(I)cc13)C2 ; CC(C)(C)OC(=O)N1C2CCC1CN(c1nc(OCC3(CN4CCOCC4)CC3)nc3c(F)c(Br)c(C(F)(F)F)cc13)C2 ; COCOc1cc(-c2c(C(F)(F)F)cc3c(N4CC5CCC(C4)N5C(=O)OC(C)(C)C)nc(OCC4(CN5CCOCC5)CC4)nc3c2F)c2c(C#C[Si](C(C)C)(C(C)C)C(C)C)cccc2c1 ; C#Cc1cccc2cc(O)cc(-c3c(C(F)(F)F)cc4c(N5CC6CCC(C5)N6)nc(OCC5(CN6CCOCC6)CC5)nc4c3F)c12]

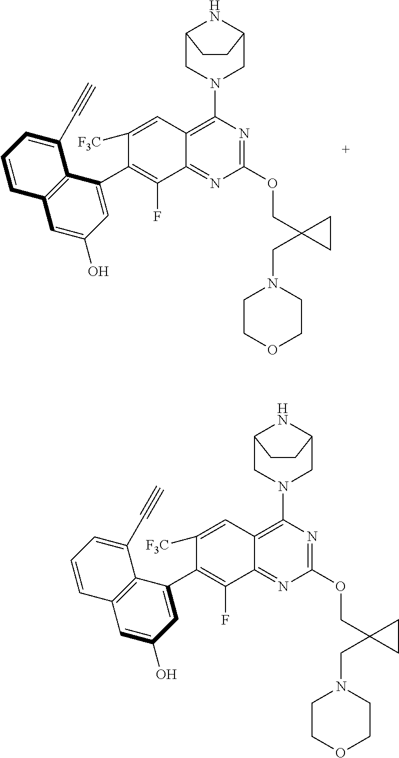

To a solution of Compound 38-2 (6.03 g, 8.23 mmol), diphenyl-(trifluoromethyl)-sulfonium trifluoromethanesulfonate (6.62 g, 16.37 mmol) in NMP (70 mL) was added Copper powder (1.57 g, 24.71 mmol). The reaction mixture was stirred at 60° C. for overnight under nitrogen atmosphere. The reaction was filtered and the filtrate concentrated under vacuum. The residue was purified by Flash (C18 column, A: 0.1% TFA in water, B: CH₃CN, Gradient: 30% B to 60% B in 30 min at a flow rate of 100 mL/min, 254 nm) to afford the desired product Compound 98-1 (3.57 g, 5.29 mmol). MS m/z: 674/676 [M+H]⁺.

To a solution of Compound 98-1 (3.57 g, 5.29 mmol), INT C12 (3.10 g, 6.27 mmol) in toluene (36 mL) and water (9 mL) were added Cs₂CO₃ (3.46 g, 10.62 mmol) and cataCXium A Pd G3 (410 mg, 0.563 mmol). The reaction mixture was stirred at 100° C. for overnight under nitrogen atmosphere. The reaction was diluted with water (50 mL) and extracted with EA (2×50 mL). The organic layers were combined, dried over Na₂SO₄ and concentrated under vacuum. The residue was purified by silica gel chromatographic column (Hex:EA=3:1, v/v) to afford the desired product Compound 98-2 (3.00 g, 2.31 mmol). MS m/z: 962 [M+H]⁺.

A solution of Compound 98-2 (3.00 g, 2.31 mmol), HCl (8 mL, 1 M in dioxane) in MeCN (35 mL) was stirred at RT for 1 h. The mixture was concentrated in vacuum. The residue was diluted with sat. NaHCO₃ (20 mL) and extracted with EA (2×20 mL). The organic layers were combined, dried over Na₂SO₄ and concentrated under vacuum. The residue in DMF (30 mL) was added CsF (4.86 g, 31.99 mmol). The reaction mixture was stirred at 40° C. for 2 hours under nitrogen atmosphere. The reaction mixture was purified by Prep-HPLC (C18 column, A: 0.1% TFA in water, B: CH₃CN, Gradient: 10% B to 40% B in 40 min at a flow rate of 40 mL/min, 242 nm) to afford the desired product Compound 98 (1.00 g, 1.12 mmol, 2TFA salt). MS m/z: 662 [M+H]⁺

The two isomers were separated by chiral-HPLC separation using a CHIRALPAK-ID column (2 cm×25 cm, 5 um) on Prep-HPLC-Gilson eluting with HeX (0.1% IPA·M)/EtOH (50:50) at a flow rate of 20 mL/min to afford first peak Compound 98A (224.8 mg, Ret Time 4.536 min) and second peak Compound 98B (218.4 mg, Ret Time 5.566 min) respectively.

### Example 68

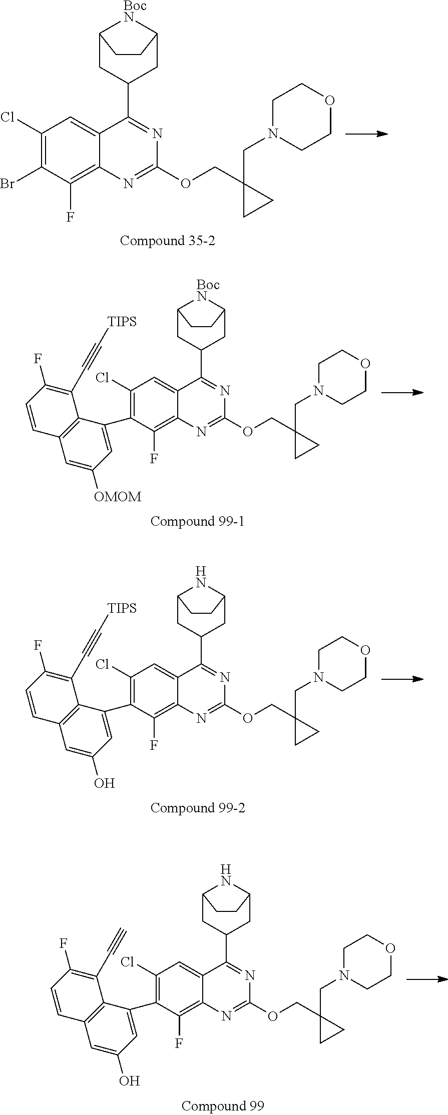

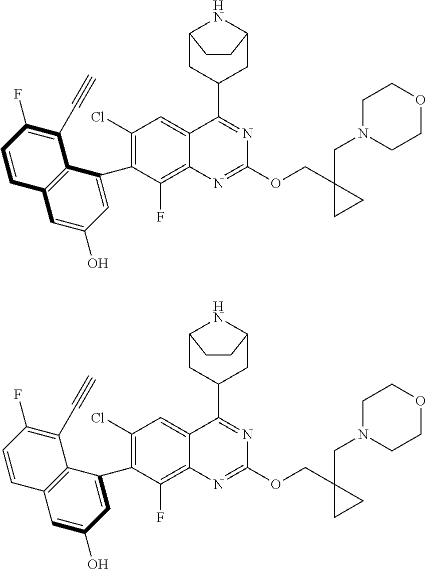

Compound 99 (Compound 99A and Compound 99B) was obtained following the procedure of Compound 35 with INT C13 and Compound 35-2 as start materials.

### Example 69

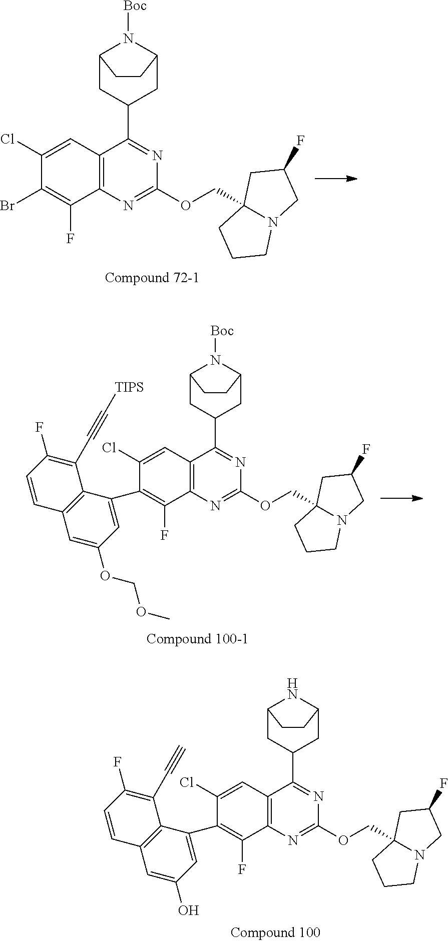

Compound 100 was obtained following the procedure of Compound 72 with INT C13 and Compound 72-1 as start materials.

### Example 70

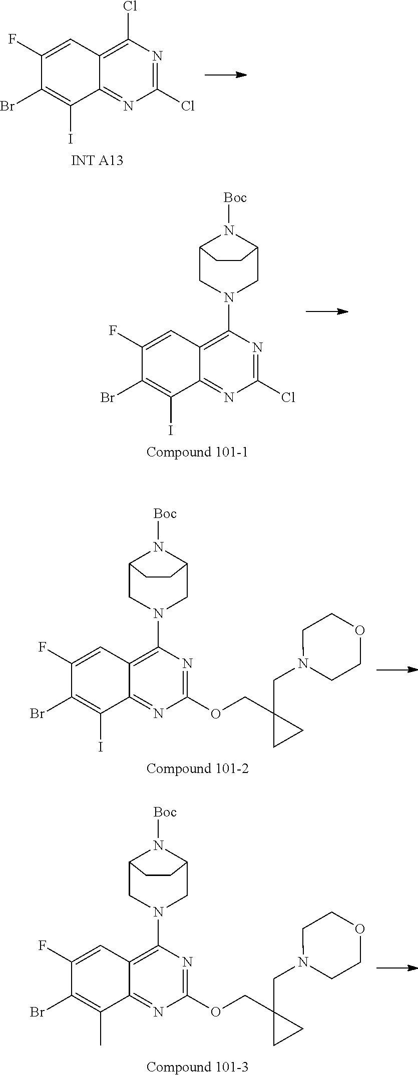

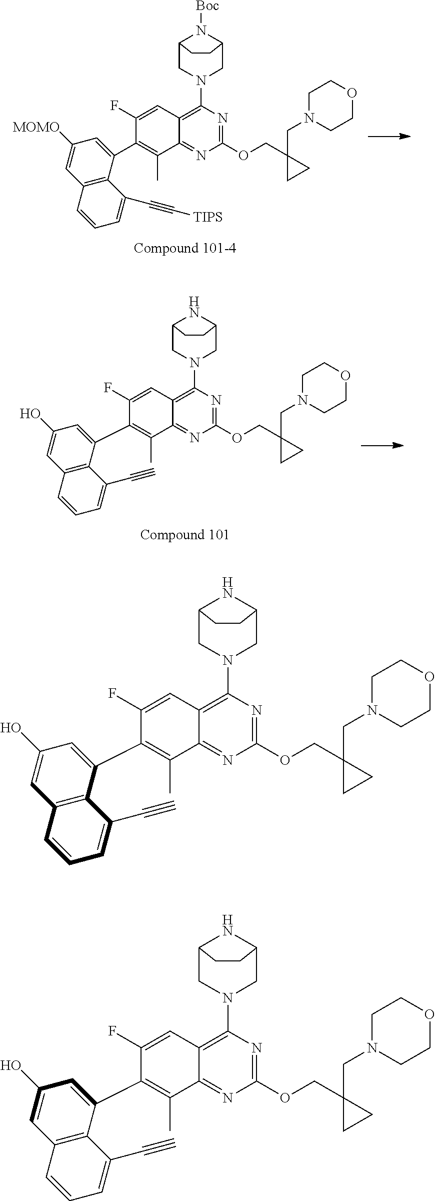

Compound 101 (Compound 101A and Compound 101B) was obtained following the procedure of Compound 38 with INT A13 as start material.

### Example 71

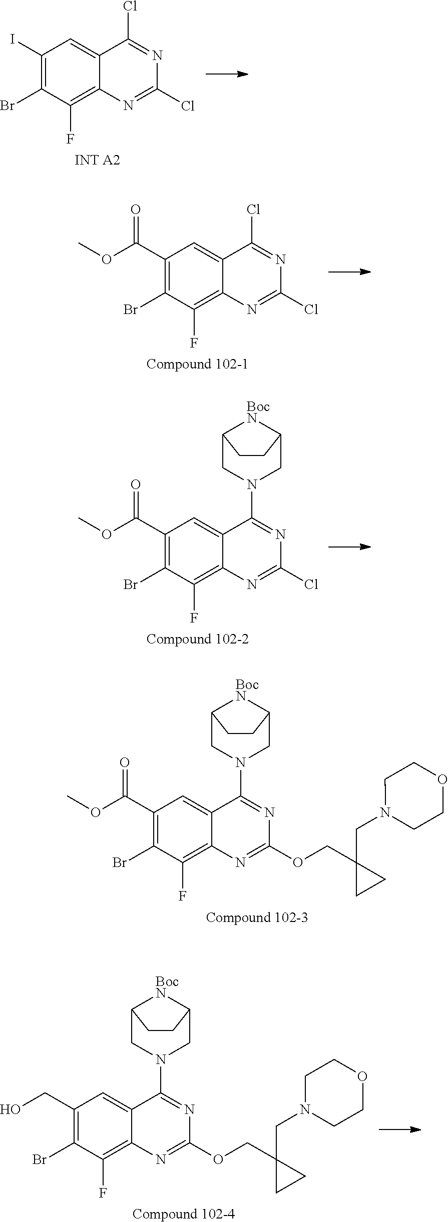

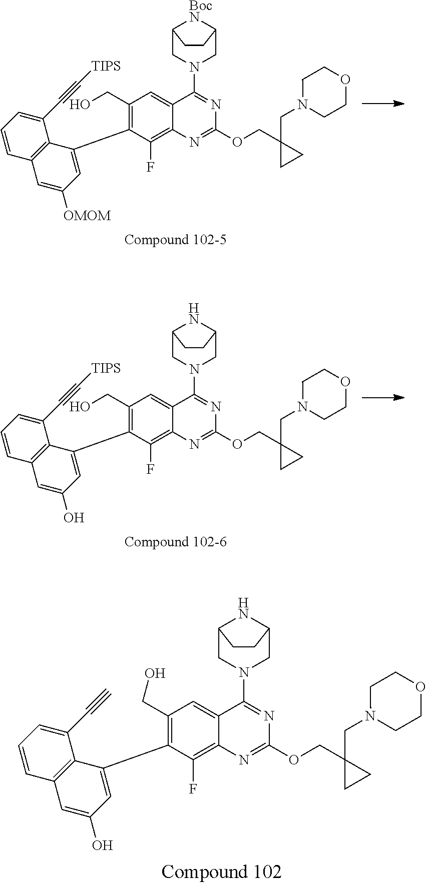

Compound 102 was obtained following the procedure of Compound 38 with INT A2 as start material.

### Example 72

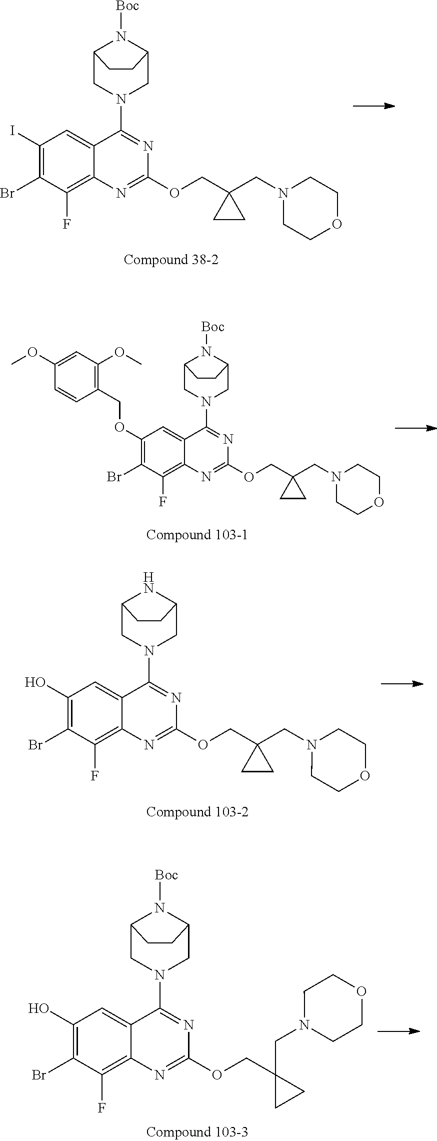 [SMILES: CC(C)(C)OC(=O)N1C2CCC1CN(c1nc(OCC3(CN4CCOCC4)CC3)nc3c(F)c(Br)c(I)cc13)C2 ; COc1ccc(COc2cc3c(N4CC5CCC(C4)N5C(=O)OC(C)(C)C)nc(OCC4(CN5CCOCC5)CC4)nc3c(F)c2Br)c(OC)c1 ; Oc1cc2c(N3CC4CCC(C3)N4)nc(OCC3(CN4CCOCC4)CC3)nc2c(F)c1Br ; CC(C)(C)OC(=O)N1C2CCC1CN(c1nc(OCC3(CN4CCOCC4)CC3)nc3c(F)c(Br)c(O)cc13)C2]

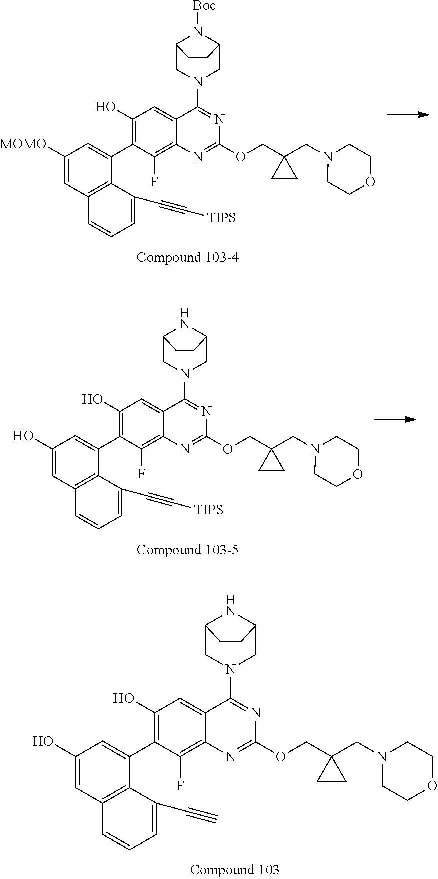

Compound 103 was obtained following the procedure of Compound 38 with Compound 38-2 as start material.

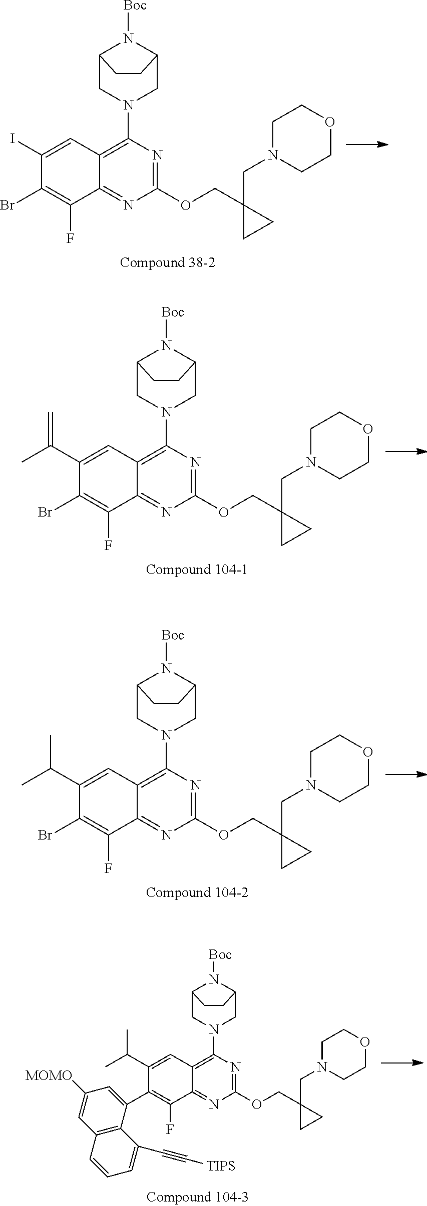

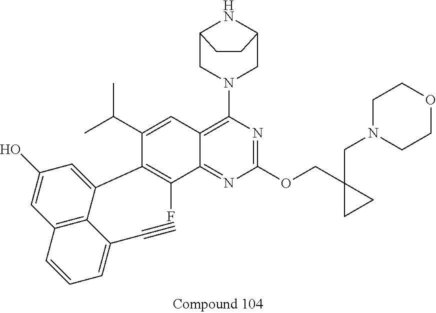 [SMILES: C#Cc1cccc2cc(O)cc(-c3c(C(C)C)cc4c(N5CC6CCC(C5)N6)nc(OCC5(CN6CCOCC6)CC5)nc4c3F)c12]

### Example 73

To a solution of Compound 38-2 (501 mg, 0.68 mmol), 4,4,5,5-Tetramethyl-2-(prop-1-en-2-yl)-1,3,2-dioxaborolane (187 mg, 1.11 mmol) in 1,4-dioxane (10 mL) were added K₃PO₄ (477 mg, 2.25 mmol) and Pd(dppf)Cl₂—CH₂Cl₂ (59 mg, 0.073 mmol). The reaction mixture was stirred overnight at 80° C. under nitrogen atmosphere. The reaction was filtered and the filtrate concentrated under reduced pressure. The residue was purified by Pre-TLC (Hex:EA=2:1, v/v) to afford the desired product Compound 104-1 (352 mg, 0.544 mmol). MS m/z: 646/648 [M+H]⁺.

To a solution of Compound 104-1 (288 mg, 0.445 mmol) in MeOH/EA (25 mL/20 mL, v/v) was added Pt/C (39 mg, 0.010 mmol, 5% wt). The reaction mixture was stirred overnight at RT under H₂ atmosphere. The reaction was filtered and concentrated under reduced pressure. The residue was purified by Pre-TLC (Hex:EA=2:1, v/v) to afford the desired product Compound 104-2 (119 mg, 0.183 mmol). MS m/z: 648/650 [M+H]⁺.

To a solution of Compound 104-2 (119 mg, 0.183 mmol), INT C12 (101 mg, 0.204 mmol) in toluene (4 mL) and water (1 mL) were added Cs₂CO₃ (121 mg, 0.371 mmol) and cataCXium A Pd G₃ (14 mg, 0.019 mmol). The reaction mixture was stirred overnight at 100° C. under nitrogen atmosphere. The reaction was diluted with water (20 mL) and extracted with EA (2×20 mL). The organic layers were combined, dried over Na₂SO₄ and concentrated under reduced pressure. The residue was purified by Pre-TLC (Hex:EA=3:1, v/v) to afford the desired product Compound 104-3 (121 mg, 0.045 mmol, 35% purity). MS m/z: 936 [M+H]⁺.

A solution of Compound 104-3 (121 mg, 0.045 mmol, 35% purity), HCl (1 mL, 4 M in dioxane) in MeCN (3 mL) was stirred at RT for 1 h. The mixture was concentrated under reduced pressure. The residue was diluted with sat. NaHCO₃ (20 mL) and then extracted with EA (2×20 mL). The organic layers were combined, dried over Na₂SO₄ and concentrated under reduced pressure. The residue dissolved in DMF (3 mL) and CsF (0.20 g, 1.32 mmol) was added. The reaction mixture was stirred overnight at 40° C. under nitrogen atmosphere. The reaction mixture was purified by Prep-HPLC (Durashell C18 column, A: 0.1% NH₃·H₂O in water, B: CH₃CN, Gradient: 20% B to 55% B to 80% B in 60 min at a flow rate of 40 mL/min, 238 nm) to afford Compound 104 (4.20 mg, 0.007 mmol). MS m/z: 636 [M+H]⁺.

### Example 74

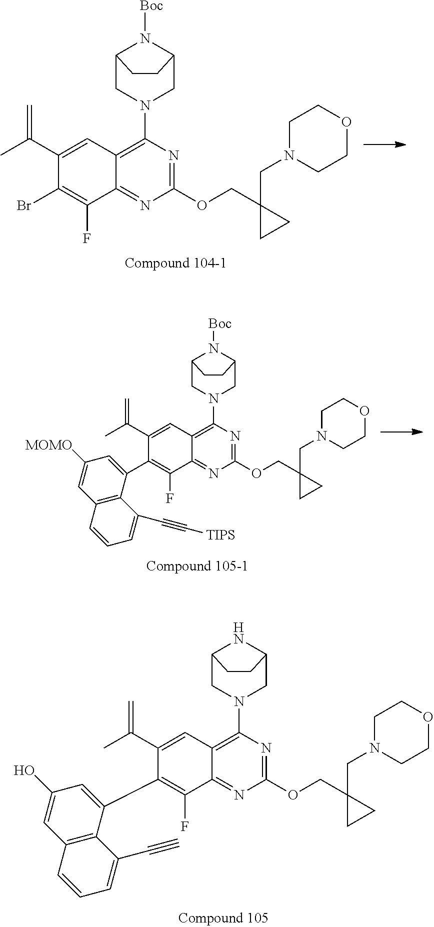

To a solution of Compound 104-1 (113 mg, 0.17 mmol), INT C12 (109 mg, 0.22 mmol) in toluene (4 mL) and water (1 mL) were added Cs₂CO₃ (124 mg, 0.38 mmol) and cataCXium A Pd G₃ (27 mg, 0.037 mmol). The reaction mixture was stirred overnight at 100° C. under nitrogen atmosphere. The reaction was diluted with water (40 mL) and extracted with EA (2×40 mL). The organic layers were combined, dried over Na₂SO₄ and concentrated under reduced pressure. The residue was purified by Pre-TLC (Hex:EA=1.5:1, v/v) to afford the desired product Compound 105-1 (49 mg, 0.052 mmol, 30% purity). MS m/z: 934 [M+H]⁺.

A solution of Compound 105-1 (49 mg, 0.052 mmol), HCl (1 mL, 4 M in dioxane) in MeCN (3 mL) was stirred at RT for 40 minutes. The mixture was concentrated under reduced pressure. The residue was diluted with sat. NaHCO₃ (20 mL) and then extracted with EA (2×20 mL). The organic layers were combined, dried over Na₂SO₄ and concentrated under reduced pressure. The residue dissolved in DMF (3 mL) and CsF (0.37 g, 2.44 mmol) was added. The reaction mixture was stirred overnight at 40° C. under nitrogen atmosphere. The reaction was filtered and the filtrate concentrated under reduced pressure. The residue was purified by Prep-HPLC to afford Compound 105 (1.80 mg, 2.7%). MS m/z: 634 [M+H]⁺.

### Example 75

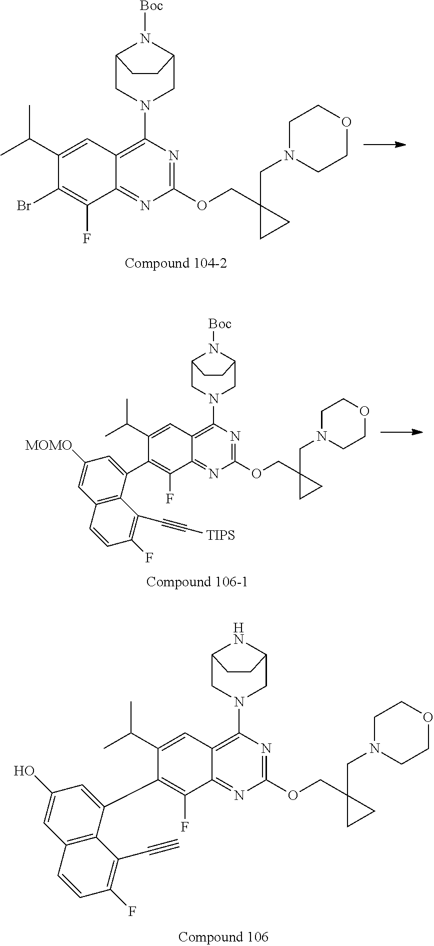

Compound 106 was obtained following the procedure of Compound 104 with Compound 104-2 and INT C13 as start materials.

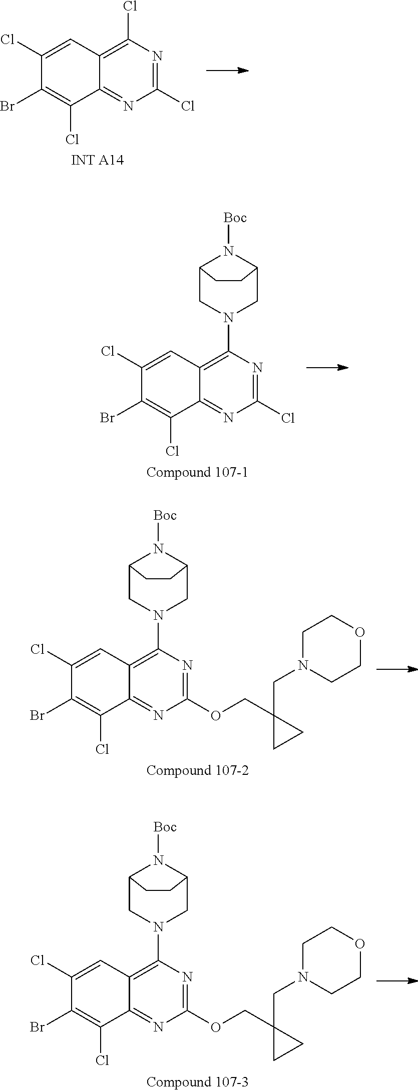

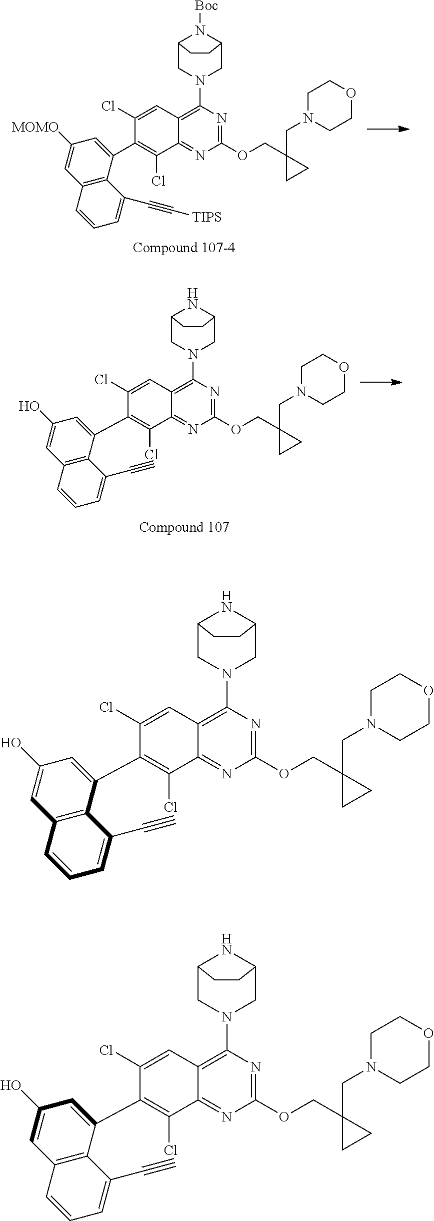

### Example 76

Compound 107 (Compound 107A and Compound 107B) was obtained following the procedure of Compound 35 with INT A14 as start material.

### Example 77

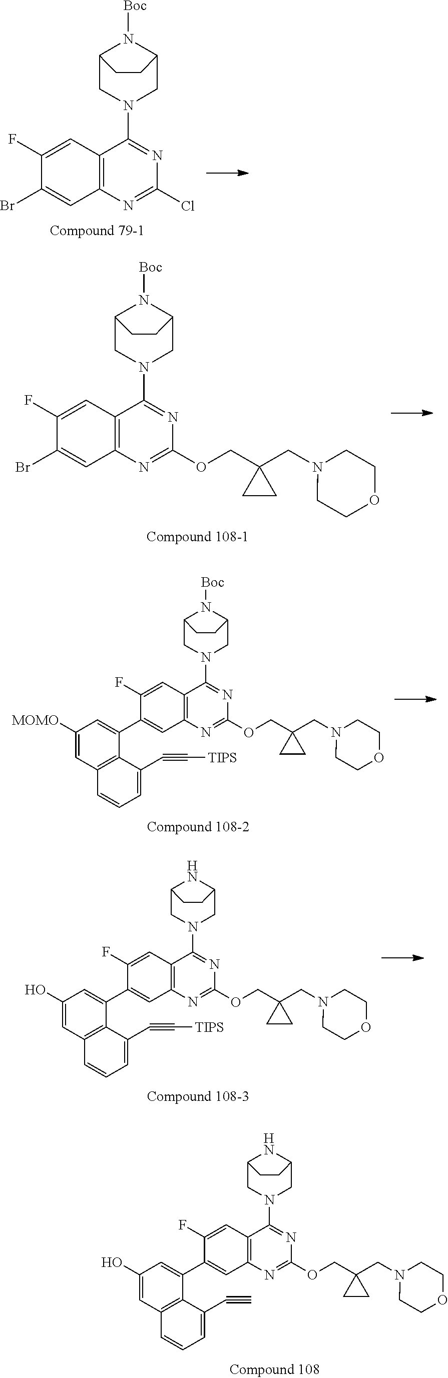

Compound 79-1 (500.0 mg, 1.06 mmol, 1.0 eq), INT B2 (362.5 mg, 2.12 mmol, 2.0 eq), DABCO (59.4 mg, 0.53 mmol, 0.5 eq), and Cs₂CO₃ (1.04 g, 3.18 mmol, 3.0 eq) were dispersed in a mixed solution of THF and DMF (V_{TH}F/V_{DMF}=8.5 ml/8.5 ml). The reaction mixture was stirred at R.T for 18 hrs, diluted with water (20 mL), and then extracted with EtOAc (10 mL×3). The organic layers were combined, dried over Na₂SO₄, concentrated to obtain a residue which was purified with column chromatography (petroleum ether/ethyl acetate=6:1) to afford Compound 108-1 (350.0 mg, 51.1% yield) as a yellow solid. LCMS: 606 ([M+H]⁺).

Compound 108-1 (290.0 mg, 0.48 mmol, 1.0 eq), INT C12 (307.4 mg, 0.62 mmol, 1.3 eq), K₃PO₄ (304.4 mg, 1.43 mmol, 3.0 eq), cataCXium A Pd G₃ (52.2 mg, 0.07 mmol, 0.15 eq) and a mixed solution of 1,4-dioxane and H₂O (V_{1,4-dioxane}/V_{H2O}=2.4 ml/0.5 ml) were placed in an oven-dried flask. The reaction mixture was stirred for 1 h at 60° C. under N₂ atmosphere, cooled to R.T, diluted with water (1 mL), and then extracted with EtOAc (1 mL×3). The organic layers were combine, dried over Na₂SO₄, and concentrated to obtain a residue which was purified with silica gel chromatography (petroleum ether/ethyl acetate=5:1) to afford Compound 108-2 (220.0 mg, 51.5% yield) as yellow oil. LCMS: 894 ([M+H]⁺).

HCl/dioxane (4 M, 2 ml) was added dropwise for 1 h to a solution of Compound 108-2 (200 mg, 0.22 mmol, 1.0 eq) in MeCN (20 mL) at 0° C. The reaction mixture was concentrated to afford a crude product containing Compound 108-3 (300 mg) as yellow oil which was used for next step without any further purification. LCMS: 750 ([M+H]⁺).

CsF (931.6 mg, 6.13 mmol, 20.0 eq) was added to a solution of the crude product containing Compound 108-3 (230 mg, 0.31 mmol, 1.0 eq) in DMF (230 ml). The reaction mixture was stirred overnight at 40° C., diluted with water (20 mL), then extracted with EtOAc (20 mL×3). The organic layers were combined, dried over Na₂SO₄, concentrated to obtain a residue which was purified with pre-HPLC to afford Compound 108 (34.4 mg, 38.2%) as a white solid. ¹H NMR (400 MHz, DMSO-d₆): δ7.89 (d, J=8.0 Hz, 1H), 7.58 (d, J=10.4 Hz, 1H), 7.48-7.46 (m, 2H), 7.43-7.40 (m, 1H), 7.31-7 (d, J=2.4 Hz, 1H), 7.01 (d, J=2.4 Hz, 1H), 4.25-4.14 (m, 4H), 3.60 (s, 1H), 3.54 (m, 6H), 3.46-3.44 (m, 2H), 2.39 (s, 1H), 2.31-2.20 (m, 7H), 1.72-1.68 (m, 4H), 0.62-0.61 (m, 2H), 0.40-0.39 (m, 2H). LCMS: 594.2 ([M+H]⁺).

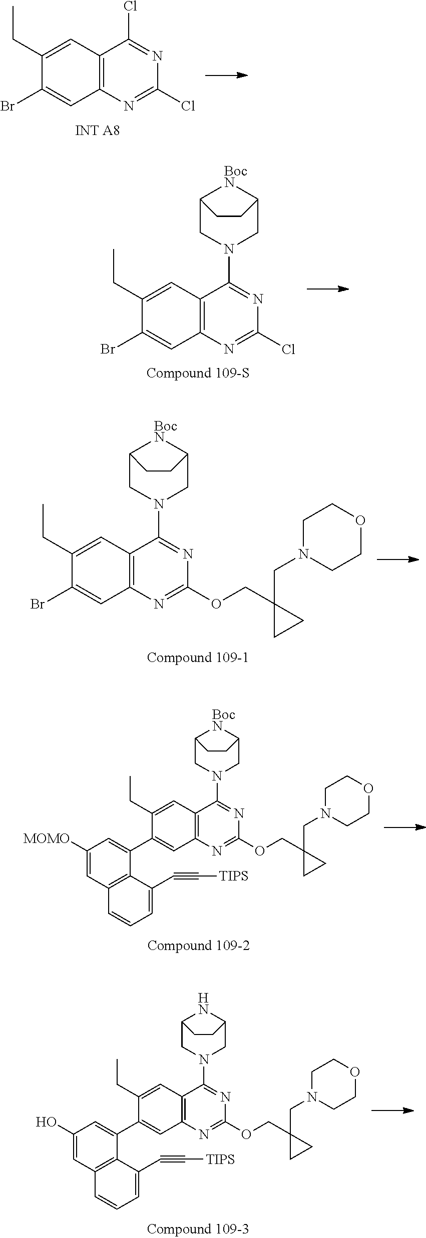

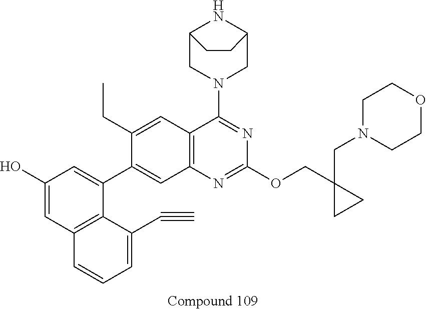 [SMILES: C#Cc1cccc2cc(O)cc(-c3cc4nc(OCC5(CN6CCOCC6)CC5)nc(N5CC6CCC(C5)N6)c4cc3CC)c12]

Tert-butyl-3,8-diazabicyclo[3.2.1]octane-8-carboxylate (413.8 mg, 1.95 mmol, 1.3 eq) was added to a solution of the crude product containing INT A8 and TEA (592.6 mg, 5.58 mmol, 3.9 eq) in 1,4-dioxane (3.5 mL). The reaction mixture was stirred at R.T for 7 hrs, diluted with H₂O (6 mL), and extracted with DCM (6 mL×3). The organic layers were combined, dried over Na₂SO₄ and concentrated to obtain a residue which was purified by silica gel chromatography (Petroleum ether/Ethyl acetate=10:1 to 5:1) to afford Compound 109-S (570 mg, 1.2 mmol, 79.1% yield) as a yellow solid. LCMS: 481.1 ([M+H]⁺).

DABCO (34 mg, 0.2 mmol, 0.5 eq) and Cs₂CO₃ (391.0 mg, 1.2 mmol, 3.0 eq) were added to a solution of Compound 109-S (200 mg, 0.4 mmol, 1.0 eq) and INT B2 (142.2 mg, 0.8 mmol, 2.0 eq) in DMF (2 mL) and THF (2 mL). The reaction mixture was stirred at 60° C. for 7 hrs under N₂ atmosphere, diluted with water (10 mL), then extracted with EtOAc (10 mL×3). The organic layers were combined, dried over Na₂SO₄, and concentrated to obtain a residue which was purified with silica gel chromatography (petroleum ether/ethyl acetate=20:1 to 5:1) to afford Compound 109-1 (200 mg, 0.33 mmol, 40.7% yield) as a yellow liquid. LCMS: 616.2 ([M+H]⁺).

A mixture of Compound 109-1 (110 mg, 0.2 mmol, 1.0 eq), INT C12 (292 mg, 0.6 mmol, 3.0 eq), K₃PO₄ (127.4 mg, 0.6 mmol, 3.0 eq), cataCXium A Pd G3 (21.8 mg, 0.03 mmol, 0.15 eq) and a mixed solution of THF and H₂O (V_{THF}:V_{H2O}=4:1, 12 mL) was purged with argon, stirred at 60° C. for 1 h, diluted with water (20 mL), and then extracted with EtOAc (20 mL×3). The organic layers were combined, washed with saturated brine, dried over anhydrous Na₂SO₄, and concentrated to obtain a residue which was purified with pre-TLC to afford Compound 109-2 (110 mg, 0.12 mmol, 40.5% yield) as a yellow solid. LCMS: 904.0 ([M+H]⁺).

HCl/dioxane (4 M, 1 ml) was added to a solution of Compound 109-2 (110 mg, 0.12 mmol, 1.0 eq) in MeCN (4.5 mL) at 0° C. The reaction mixture was stirred at 0° C. for 2.5 hrs, and then concentrated to afford a crude product containing Compound 109-3 as a yellow solid which was used directly for the next step without any further purification. LCMS: 760.4 ([M+H]⁺).

A mixture of the crude product containing Compound 109-3, CsF (273.4 mg, 1.8 mmol, 15.0 eq) and DMF (4.0 ml) was stirred overnight at 50° C., and then purified by pre-HPLC to afford Compound 109 (22.4 mg, 0.037 mmol, 30.9% yield) as a white solid. ¹H NMR (300 MHz, DMSO-d₆): δ 10.03 (s, 1H), 7.93-7.77 (m, 1H), 7.64 (s, 1H), 7.47-7.32 (m, 2H), 7.25 (d, J=3.7 Hz, 2H), 6.90 (d, J=2.5 Hz, 1H), 4.26 (d, J=11.6 Hz, 1H), 4.19 (s, 2H), 4.10 (d, J=12.0 Hz, 1H), 3.53 (s, 6H), 3.49-3.39 (m, 2H), 3.36 (s, 2H), 2.37 (s, 4H), 2.32-2.23 (m, 3H), 1.84-1.64 (m, 4H), 1.00 (t, J=7.5 Hz, 3H), 0.60-0.59 (m, 2H), 0.39-0.38 (m, 2H). LCMS: 603.3 ([M+H]⁺).

### Example 79

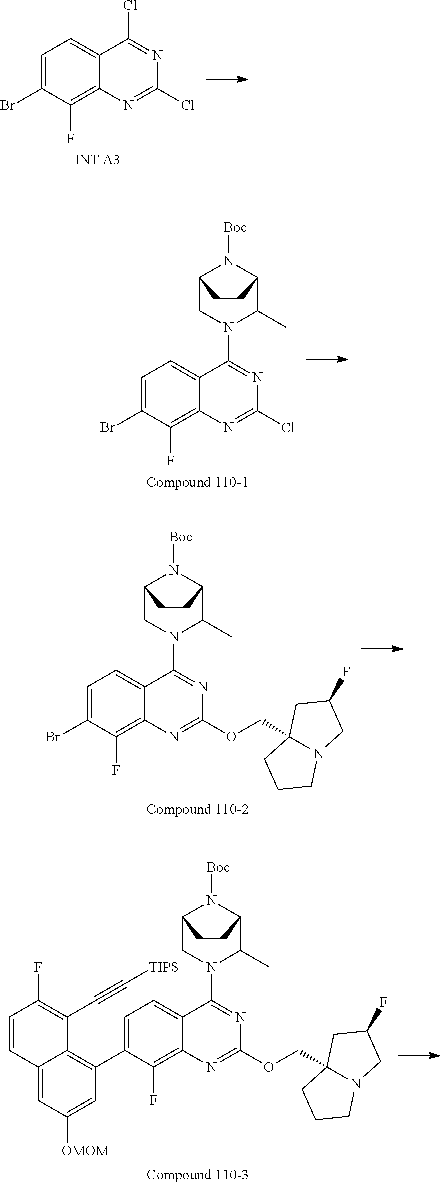

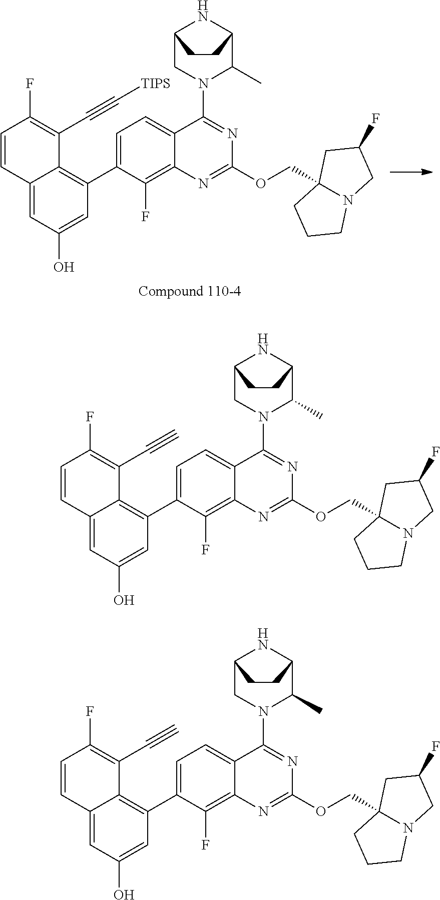

Compound 110 (Compound 110A and Compound 110B) were obtained following the procedure of Compound 67 with INT A3 as start material.

### Example 80

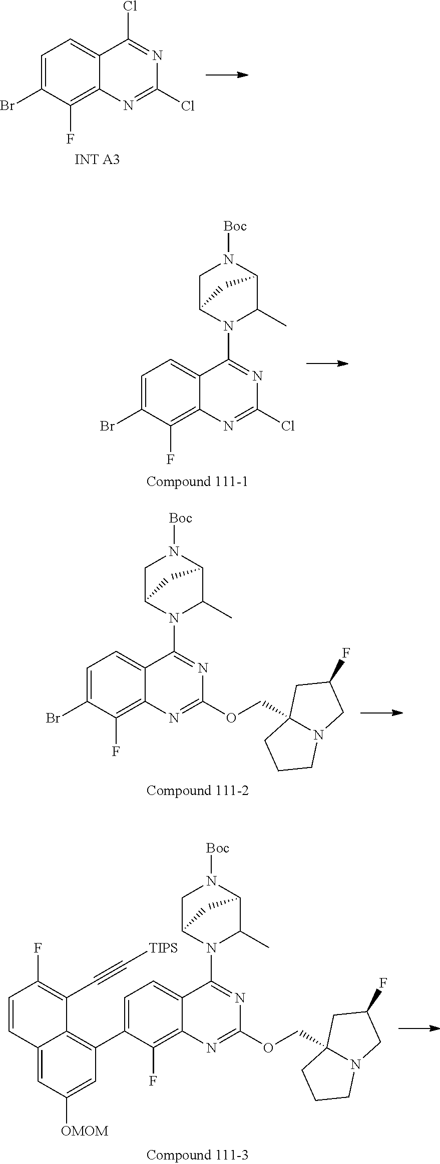

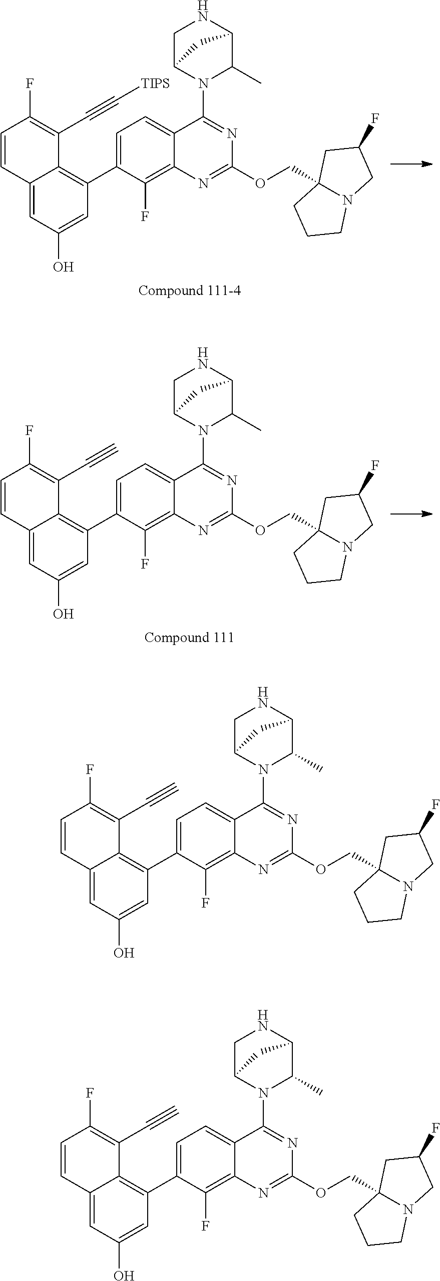

Compound 111 (Compound 111A and Compound 111B) were obtained following the procedure of Compound 67 with INT A3 as start material.

### Example 81

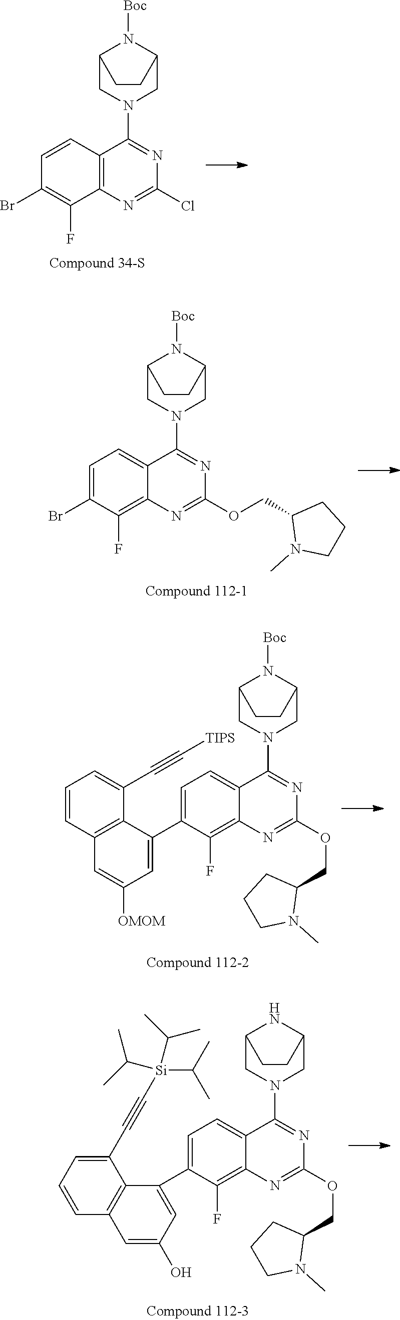

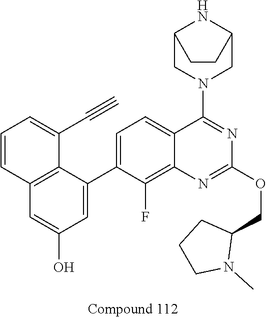 [SMILES: C#Cc1cccc2cc(O)cc(-c3ccc4c(N5CC6CCC(C5)N6)nc(OC[C@@H]5CCCN5C)nc4c3F)c12]

Compound 112 was obtained following the procedure of Compound 34 with Compound 34-S as start material.

### Example 82

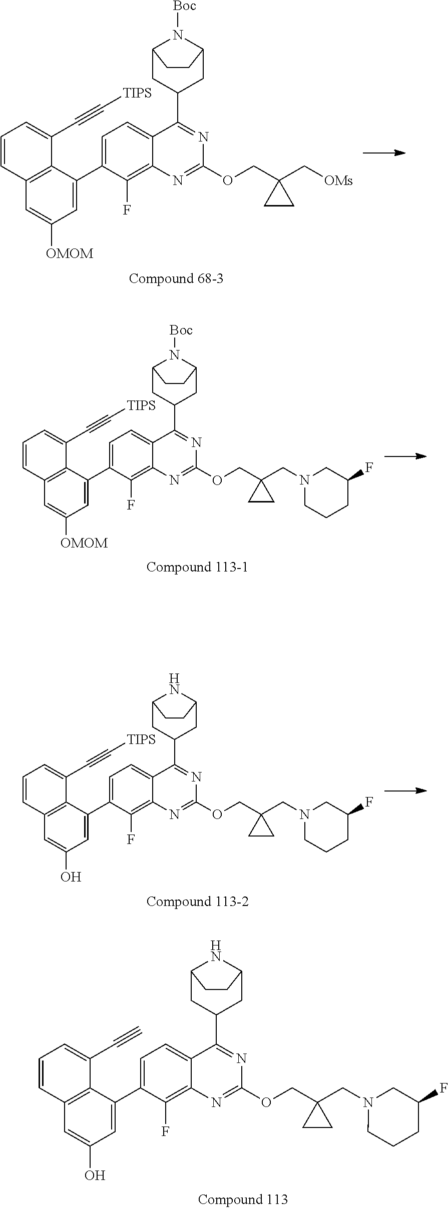

TEA (87.0 mg, 0.9 mmol, 5.0 eq) were added to a solution of (3S)-3-fluoropiperidine hydrochloride (36.0 mg, 0.3 mmol, 1.5 eq) in DMF (2 mL), after stirring for 2 hrs, Compound 68-3 (155.0 mg, 0.2 mmol, 1.0 eq) was added. The reaction mixture was stirred for 2 hrs at 80° C., cooled to R.T, poured into water (5 mL) and extracted with EtOAc (5 mL×2). The organic layers were combined, washed with water (5 mL) and brine (5 mL) successively, dried with anhydrous Na₂SO₄, and concentrated to obtain a residue which was purified with Pre-TLC (eluted with DCM/MeOH=10:1) to afford Compound 113-1 (39.0 mg, yield 24.8%) as a yellow liquid. LCMS: 910 ([M+H]⁺).

A solution of Compound 113-1 (50.0 mg, 0.05 mmol, 1.0 eq) and HCl/1,4-dioxane (3.5 mL) at room temperature in MeCN (5 mL) was stirred for 1 h at 30° C., and then concentrated to afford a crude product containing Compound 113-2 (60.0 mg) as a yellow solid. LCMS: 766 ([M+H]⁺).

A mixture of the crude product containing Compound 113-2 (60.0 mg), CsF (122.0 mg, 0.8 mmol, 10.0 eq) and DMF (3 mL) was stirred for 3 hrs at 60° C., and then filtered. The filtrate was purified with Prep-HPLC to afford Compound 113 (5.5 mg) as a white solid. ¹H NMR (400 MHz, CD₃OD-d₄): δ 7.79-7.72 (m, 2H), 7.48-7.47 (m, 1H), 7.38-7.34 (m, 1H), 7.26-7.19 (m, 2H), 7.03 (d, J=2.4 Hz, 1H), 4.58-4.33 (m, 6H), 4.37 (s, 2H), 3.63-3.59 (m, 2H), 2.85 (d, J=2.4 Hz, 2H), 2.65-2.54 (m, 2H), 2.50-2.41 (m, 3H), 1.96-1.91 (m, 5H), 1.81-1.79 (m, 2H), 0.72-0.70 (m, 2H), 0.50-0.49 (m, 2H). LCMS: 610.3 ([M+H]⁺).

### Example 83

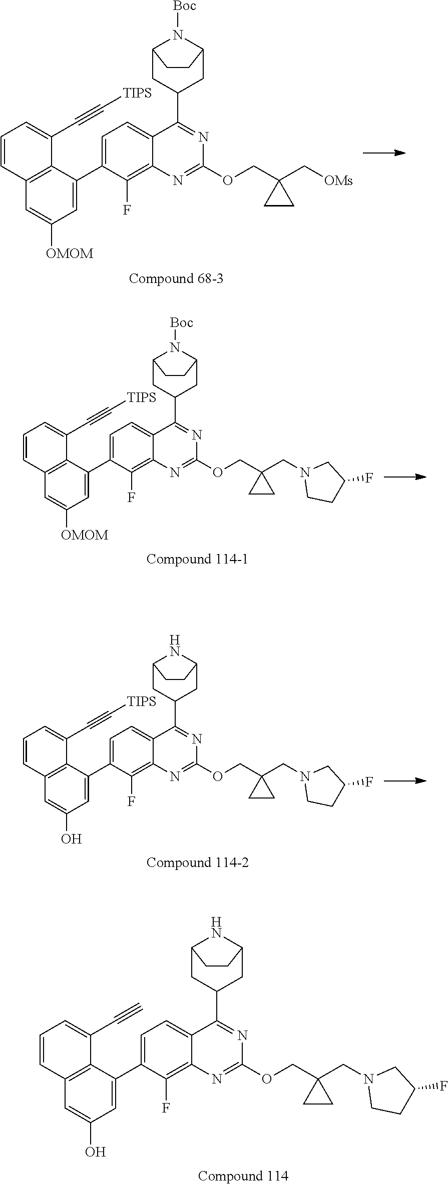

TEA (76.0 mg, 0.8 mmol, 5.0 eq) was added to a solution of (R)-3-fluoropyrrolidin (20.0 mg, 0.2 mmol, 1.5 eq) in DMF (2 mL), after stirring for 2 hrs at R.T, Compound 68-3 (135.0 mg, 0.2 mmol, 1.0 eq) was added. The reaction mixture was stirred for 2 hrs at 80° C., cooled to R.T, poured into water (5 mL) and extracted with EtOAc (5 mL×2). The organic layers were combined, washed with water (5 mL) and brine (5 mL) successively, dried with anhydrous Na₂SO₄, and concentrated to obtain a residue which was purified with Pre-TLC (eluting with DCM/MeOH=10:1) to afford Compound 114-1 (77.5 mg, yield 50.4%) as a yellow liquid. LCMS: 896 ([M+H]⁺).

A solution of Compound 114-1 (100.0 mg, 0.1 mmol, 1.0 eq) and HCl/Dioxane (4 M, 3.5 mL) in MeCN (5 mL) was stirred for 1 h at R.T, and then concentrated to afford a crude product containing Compound 114-2 (110 mg) as a yellow solid which was used directly for next step without any further purification. LCMS: 752 ([M+H]⁺).

A mixture of the crude product containing Compound 114-2 (110.0 mg crude, 0.15 mmol, 1.0 eq), CsF (228.0 mg, 1.5 mmol, 10.0 eq) and DMF (3 mL) was stirred for 3 hrs at 60° C., and then filtered. The filtrate was purified with Prep-HPLC to afford Compound 114 (15 mg) as a white solid. ¹H NMR (400 MHz, CD₃OD-d₄): δ 7.79-7.72 (m, 2H), 7.48-7.47 (m, 1H), 7.38-7.34 (m, 1H), 7.26-7.20 (m, 2H), 7.03 (d, J=2.4 Hz, 1H), 5.22-5.08 (m, 1H), 4.54-4.42 (m, 3H), 4.37-4.30 (m, 1H), 3.76 (s, 2H), 3.65-3.60 (m, 2H), 2.99-2.93 (m, 2H), 2.87-2.72 (m, 2H), 2.59-2.54 (m, 2H), 2.19-2.17 (m, 2H), 1.97-1.91 (m, 5H), 0.72-0.71 (m, 2H), 0.55-0.53 (m, 2H). LCMS: 596.3 ([M+H]⁺).

### Example 84

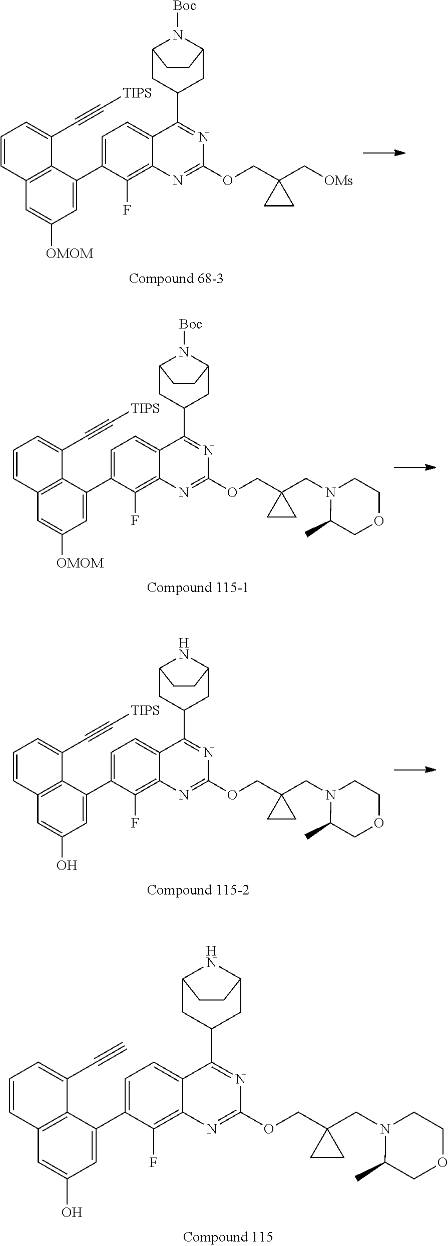

Compound 68-3 (200.0 mg, 0.22 mmol, 1.0 eq), (R)-3-methylmorpholine (44.7 mg, 0.44 mmol, 2.0 eq), TEA (67.1 mg, 0.66 mmol, 3.0 eq) were added successively into DMF (2 mL). The reaction mixture was stirred for 2 hrs at 80° C., cooled to R.T, poured into water (5 mL) and extracted with EtOAc (5 mL×2). The organic layers were combined, washed with water (5 mL) and brine (5 mL) successively, dried with anhydrous Na₂SO₄, and concentrated to obtain a residue which was purified with Pre-TLC (eluted with ether/EtOAc=2:1) to afford Compound 115-1 (45 mg, yield 19.8%) as yellow oil. LCMS: 908 ([M+H]⁺).

A solution of Compound 115-1 (45 mg, 0.05 mmol, 1.0 eq) and HCl/Dioxane (4M, 0.4 mL) in MeCN (2 mL) was stirred for 2 hrs at R.T, and then concentrated to afford a crude product containing Compound 115-2 (40 mg) as a yellow solid. LCMS: 764 ([M+H]⁺).

A mixture of the crude product containing Compound 115-2 (40 mg crude, 0.05 mmol, 1.0 eq), CsF (120 mg, 0.5 mmol, 10.0 eq) and DMF (3 mL) was stirred for 3 hrs at 40° C., and then filtered. The filtrate was purified with Prep-HPLC to afford Compound 115 (3.5 mg, yield 13.0%) as a white solid. ¹H NMR (400 MHz, CD₃OD-d₄): δ 7.79 (d, J=8.0 Hz, 1H), 7.74 (d, J=8.4 Hz, 1H), 7.48 (d, J=7.2 Hz, 1H), 7.36 (t, J=8.0 Hz, 1H), 7.27-7.24 (m, 2H), 7.03-7.02 (m, 1H), 4.69 (t, J=12.0 Hz, 1H), 4.60-4.52 (m, 1H), 4.50-4.45 (m, 1H), 4.09 (t, J=10.8 Hz, 1H), 3.84 (s, 2H), 3.76-3.70 (m, 1H), 3.66-3.57 (m, 4H), 3.48-3.34 (m, 1H), 3.19-3.09 (m, 2H), 2.85 (d, J=7.2 Hz, 1H), 2.40-2.30 (m, 1H), 2.29-2.20 (m, 1H), 2.02-1.95 (m, 4H), 1.74-1.70 (m, 1H), 0.94-0.90 (m, 3H), 0.74-0.73 (m, 1H), 0.65-0.67 (m, 2H), 0.42-0.41 (m, 1H). LCMS: 608 ([M+H]⁺).

### Example 85

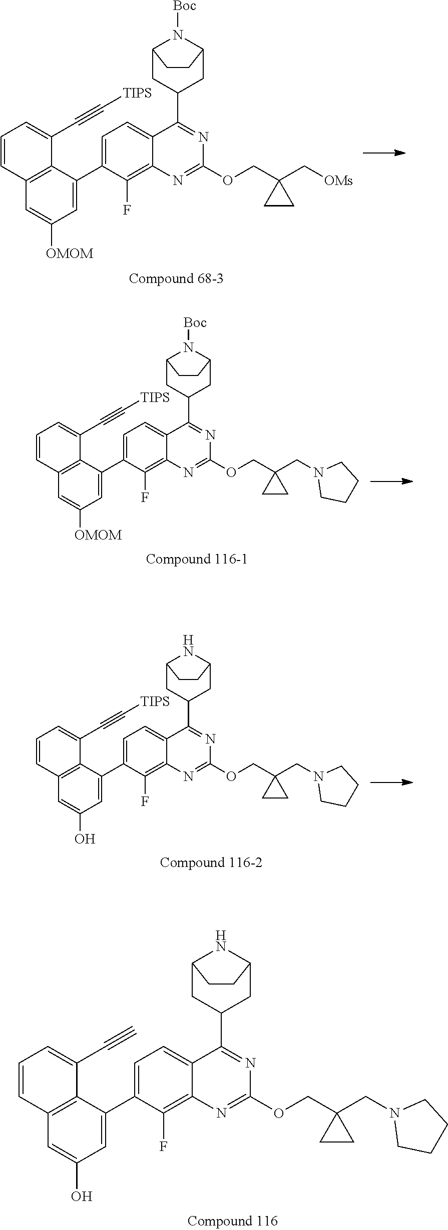

Compound 68-3 (110.0 mg, 0.12 mmol, 1.0 eq), pyrrolidine (17.2 mg, 0.24 mmol, 2.0 eq), TEA (37 mg, 0.36 mmol, 3.0 eq) were added successively into DMF (2 mL). The reaction mixture was stirred for 2 hrs at 80° C., cooled to R.T, poured into water (5 mL) and extracted with EtOAc (5 mL×2). The organic layers were combined, washed with water (5 mL) and brine (5 mL) successively, dried with anhydrous Na₂SO₄, and concentrated to obtain a residue which was purified with Pre-TLC (eluted with DCM/MeOH=10:1) to afford Compound 116-1 (70 mg, yield 66.4%) as yellow oil. LCMS: 878 ([M+H]⁺).

A solution of Compound 116-1 (100.0 mg, 0.1 mmol, 1.0 eq) and HCl/Dioxane (4 M, 1 mL) in MeCN (2 mL) was stirred at R.T for 1 h, and then concentrated to afford a crude product containing Compound 116-2 (110 mg) as a yellow solid. LCMS: 734 ([M+H]⁺).

A mixture of the crude product containing Compound 116-2 (110.0 mg), CsF (228.0 mg, 1.5 mmol, 10.0 eq) and DMF (3 mL) was stirred for 3 hrs at R.T, and then filtered. The filtrate was purified with Prep-HPLC to afford Compound 116 (15 mg, the yield of the above two steps is 23%) as a white solid. ¹H NMR (400 MHz, MeOD-d₄): δ 7.80 (d, J=8.4 Hz, 2H), 7.50-7.48 (m, 1H), 7.40-7.38 (m, 1H), 7.28-7.27 (m, 2H), 7.05 (d, J=2.4 Hz, 1H), 4.62-4.59 (m, 1H), 4.52-4.48 (m, 2H), 4.22 (d, J=12.0 Hz, 1H), 3.76-3.58 (m, 4H), 3.32-3.30 (m, 5H), 3.01 (d, J=12.4 Hz, 1H), 2.94 (s, 1H), 1.94-1.92 (m, 4H), 1.90-1.88 (m, 4H), 0.88-0.86 (m, 2H), 0.79-0.76 (m, 2H). LCMS: 578.1 ([M+H]⁺).

### Example 86

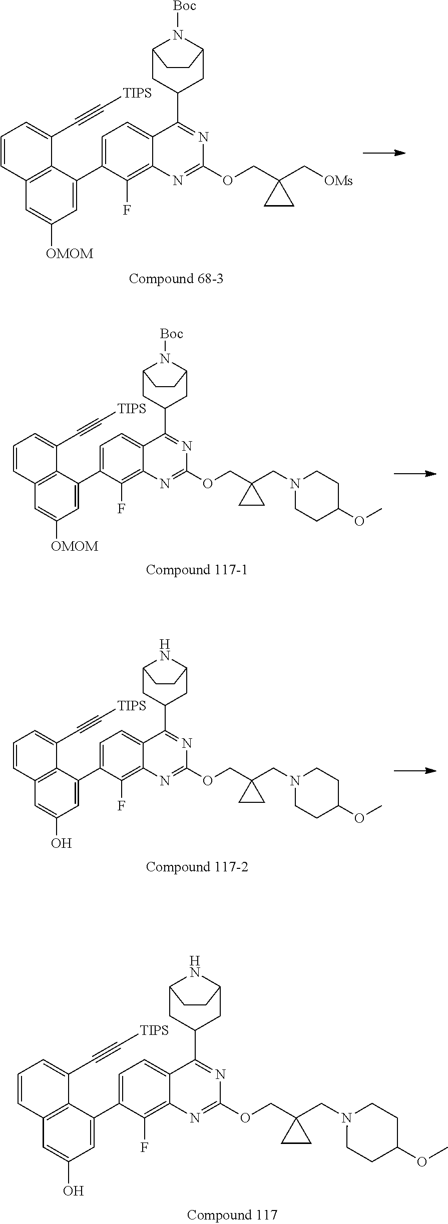

Compound 68-3 (150.0 mg, 0.16 mmol, 1.0 eq), 4-methoxypiperidine (38.3 mg, 0.33 mmol, 2.0 eq), TEA (50.3 mg, 0.48 mmol, 3.0 eq) were added successively into DMF (2 mL). The reaction mixture was stirred for 2 hrs at 80° C., cooled to R.T, poured into water (5 mL) and extracted with EtOAc (5 mL×2). The organic layers were combined, washed with water (5 mL) and brine (5 mL), dried with anhydrous Na₂SO₄, and then concentrated to obtain a residue which was purified with Pre-TLC (eluted with DCM/MeOH=10:1) to afford Compound 117-1 (70 mg, yield 45.8%) as yellow oil. LCMS: 922 ([M+H]⁺).

A solution of Compound 117-1 (110.0 mg, 0.12 mmol, 1.0 eq) and HCl/1,4-dioxane (1 mL) in MeCN (2 mL) was stirred for 2 hrs at R.T, and then concentrated to afford a crude product containing Compound 117-2 (110 mg) as a yellow solid. LCMS: 778 ([M+H]⁺).

CsF (322.0 mg, 2.1 mmol, 15.0 eq) was added to a solution of the crude product containing Compound 117-2 (110.0 mg) in DMF (3 mL) at room temperature. The reaction mixture was stirred for 3 hrs at 40° C., and then filtered. The filtrate was purified with Prep-HPLC to afford Compound 117 (9 mg, the yield of the above two steps is 12.2%) as a white solid. ¹H NMR (400 MHz, MeOD-d₄): δ 7.80-7.74 (m, 2H), 7.47 (d, J=0.8 Hz, 1H), 7.39-7.37 (m, 1H), 7.27-7.26 (m, 2H), 7.03 (d, J=2.4 Hz, 1H), 4.48-4.46 (m, 3H), 4.35-4.34 (m, 1H), 3.86-3.84 (m, 2H), 3.77-3.76 (m, 2H), 3.57-3.54 (m, 1H), 3.38-3.35 (m, 2H), 3.14-3.12 (m, 2H), 3.00 (s, 1H), 2.88-2.66 (m, 4H), 2.12-2.10 (m, 1H), 1.95-1.93 (m, 6H), 1.74-1.72 (m, 2H), 0.83-0.81 (m, 2H), 0.66-0.64 (m, 2H). LCMS: 622.3 ([M+H]⁺).

### Example 87

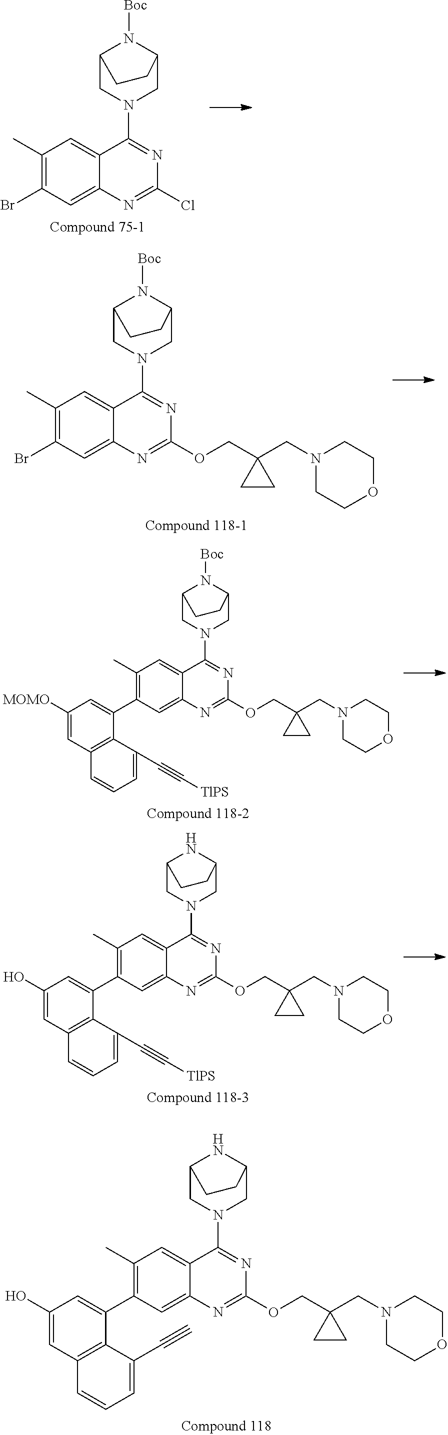

Compound 75-1 (600.0 mg, 1.3 mmol, 1.0 eq), INT B2 (438.0 mg, 2.6 mmol, 2.0 eq), DABCO (105.0 mg, 0.64 mmol, 0.5 eq), Cs₂CO₃ (1.3 g, 3.8 mmol, 3.0 eq) were added successively into a mixed solution of THF (5 mL) and DMF (5 mL). The reaction mixture was stirred for 6 hrs at 60° C., cooled to R.T, poured into water (5 mL) and extracted with EtOAc (5 mL×2). The organic layers were combined, washed with water (5 mL) and brine (5 mL) successively, dried with anhydrous Na₂SO₄, and concentrated to obtain a residue which was purified with Pre-TLC (eluting with DCM/MeOH=10:1) to afford Compound 118-1 (430 mg, yield 55.6%) as a yellow liquid. LCMS: 602.0, 603.8 ([M+H]⁺).

Compound 118-1 (370.0 mg, 0.6 mmol, 1.0 eq), INT C12 (455.0 mg, 0.9 mmol, 1.5 eq), K₃PO₄ (391.0 mg, 1.8 mmol, 3.0 eq), cataCxium A Pd G₃(67.0 mg, 0.1 mmol, 0.1 eq) were added successively into a mixed solution of THF and water (V_{THF}/V_{water}=1:1, 10 mL). The reaction mixture was purged with argon for 10 mins. The mixture was stirred for 3 hrs at 60° C. under argon atmosphere, cooled to R.T, poured into water (5 mL) and extracted with DCM (5 mL×2). The organic layers were combined, washed with water (5 mL) and brine (5 mL) successively, dried with anhydrous Na₂SO₄, and concentrated to obtain a residue which was purified with Pre-TLC (eluting with DCM/MeOH=10:1) to afford a crude product containing Compound 118-2 (240.0 mg) as a yellow solid. LCMS: 890 ([M+H]⁺).

HCl/1,4-dioxane (3.5 mL) was added to a solution of the crude product containing Compound 118-2 (280 mg, 0.3 mmol, 1.0 eq) in MeCN (5 mL) at room temperature. The reaction mixture was stirred for 1 h at 30° C., and then concentrated to afford a crude product containing Compound 118-3 (300 mg) as a yellow solid which was used for next step without further purification. LCMS: 746 ([M+H]⁺).

CsF (610.0 mg, 4.0 mmol, 10.0 eq) was added to a solution of the crude product containing Compound 118-3 (300.0 mg) in DMF (3 mL) at R.T. The reaction mixture was stirred for 3 hrs at 60° C., and then filtered. The filtrate was purified with Prep-HPLC to afford Compound 118 (66.6 mg) as a white solid. ¹H NMR (400 MHz, CD₃OD-d₄): δ 7.80-7.78 (m, 1H), 7.69 (s, 1H), 7.47-7.45 (m, 2H), 7.37-7.35 (m, 1H), 7.24 (d, J=2.4 Hz, 1H), 6.90 (d, J=2.4 Hz, 1H), 4.56-4.48 (m, 2H), 4.37 (s, 2H), 4.10 (brs, 2H), 3.73-3.65 (m, 6H), 2.83 (s, 1H), 2.58-2.52 (m, 4H), 2.51 (s, 2H), 2.19-2.10 (m, 7H), 0.72-0.70 (m, 2H), 0.53-0.51 (m, 2H). LCMS: 590.3 ([M+H]⁺).

### Example 88

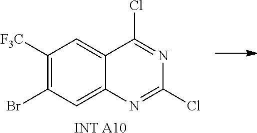

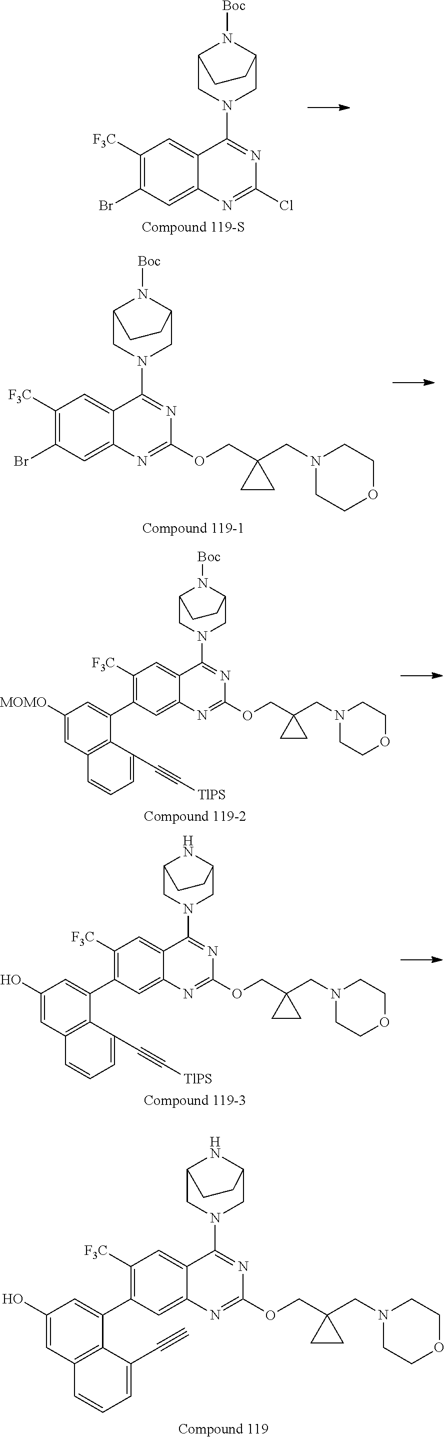

Compound 119-S was obtained following the procedure of Compound 34-S with INT A10 as start material.

Compound 119-S (420.0 mg, 0.8 mmol, 1.0 eq), INT B2 (274.0 mg, 1.6 mmol, 2.0 eq), DABCO (45.0 mg, 0.4 mmol, 0.5 eq), and Cs₂CO₃ (782.0 mg, 2.4 mmol, 3.0 eq) were added successively into a mixed solution of THF (5 mL) and DMF (5 mL). The reaction mixture was stirred for 6 hrs at 60° C., cooled to R.T, poured into water (5 mL) and extracted with EtOAc (5 mL×2). The organic layer were combined, washed with water (5 mL) and brine (5 mL) successively, dried with anhydrous Na₂SO₄, and concentrated to obtain a residue which was purified with Pre-TLC (eluting with DCM/MeOH=10:1) to afford Compound 119-1 (120.0 mg, yield 22.7%) as a white solid. LCMS: 656.2, 658.2 ([M+H]⁺).

Compound 119-1 (60.0 mg, 0.09 mmol, 1.0 eq), INT C12 (67.0 mg, 0.14 mmol, 1.5 eq), K₃PO₄ (57.0 mg, 0.27 mmol, 3.0 eq), and cataCxium A Pd G3 (10 mg, 0.01 mmol, 0.1 eq) were added successively into a mixed solution of THF and water (V_{THF}/V_{water}=1:1, 3 mL). The reaction mixture was purges with argon for 10 min, stirred for 3 hrs at 60° C. under argon atmosphere, cooled to R.T, poured into water (5 mL) and extracted with DCM (5 mL×2). The organic layers were combined, washed with water (5 mL) and brine (5 mL) successively, dried with anhydrous Na₂SO₄, and the concentrated to obtain a residue which was purified with Pre-TLC (eluting with DCM/MeOH=10:1) to afford a crude product containing Compound 119-2 (25 mg, crude) as a yellow solid. LCMS: 944.0 ([M+H]⁺).

A mixture of the crude product containing Compound 119-2 (50.0 mg), HCl/1,4-dioxane (4 M, 3.5 mL) and MeCN (5 mL) was stirred for 1 h at 30° C., and then concentrated to afford a crude product containing Compound 119-3 (60.0 mg) as a yellow solid which was used for next step without further purification. LCMS: 800.4 ([M+H]⁺).

CsF (106.0 mg, 0.7 mmol, 10.0 eq) was added to a solution of the crude product containing Compound 119-3 (60.0 mg) in DMF (3 mL) at R.T. The reaction mixture was stirred for 3 hrs at 60° C., and then filtered. The filtrate was purified with Prep-HPLC to afford Compound 119 (57.0 mg) as a white solid. ¹H NMR (400 MHz, CD₃OD-d₄): δ 8.23 (s, 1H), 7.78 (d, J=8.0 Hz, 1H), 7.49-7.45 (m, 2H), 7.37-7.33 (m, 1H), 7.25 (d, J=2.4 Hz, 1H), 6.98 (d, J=2.0 Hz, 1H), 4.60-4.57 (m, 2H), 4.38 (s, 2H), 3.94 (brs, 2H), 3.78 (t, J=14.4 Hz, 2H), 3.64 (t, J=4.4 Hz, 4H), 2.90 (s, 1H), 2.53 (brs, 4H), 2.46 (s, 2H), 2.01 (s, 4H), 0.71-0.69 (m, 2H), 0.52-0.49 (m, 2H). LCMS: 644.1 ([M+H]⁺).

### Example 89

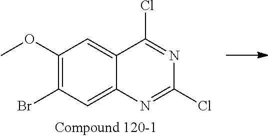

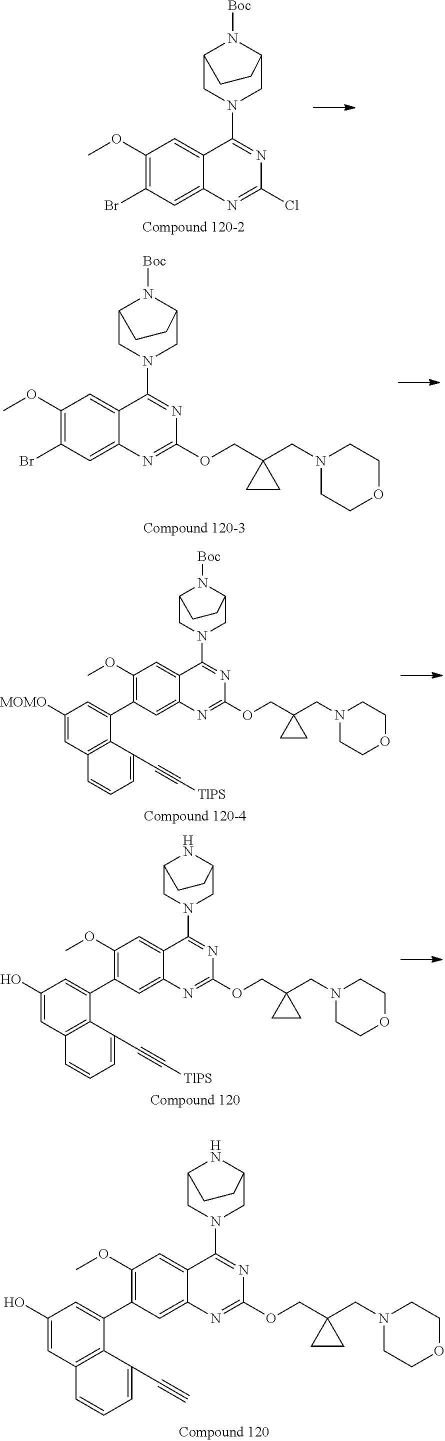

Compound 120-1 was obtained following the procedure of INT A4 with 2-amino-4-bromo-5-methoxybenzoic acid as start material.

Compound 120-1 (308.0 mg, 1.0 mmol, 1.0 eq), 8-boc-3,8-diaza-bicyclo [3.2.1] octane (212.0 mg, 1.0 mmol, 1.0 eq), TEA (303.0 mg, 3.0 mmol, 3.0 eq) were added successively into 1,4-dioxane (5 mL). The reaction mixture was stirred for 30 minutes at R.T, poured into water (5 mL) and extracted with EtOAc (5 mL). The organic layer was washed with water (5 mL) and brine (5 mL) successively, dried with anhydrous Na₂SO₄, and concentrated to obtain a residue. The residue was slurried with ether and Compound 120-2 (440.0 mg, yield 90.9%) was collected by filtration as a yellow solid. LCMS: 482.8, 484.8 ([M+H]⁺).

Compound 120-2 (310.0 mg, 0.6 mmol, 1.0 eq), INT B2 (219.0 mg, 1.3 mmol, 2.0 eq), DABCO (36.0 mg, 0.3 mmol, 0.5 eq), and Cs₂CO₃ (626.0 mg, 1.9 mmol, 3.0 eq) were added successively into a mixed solution of THF (5 mL) and DMF (5 mL). The reaction mixture was stirred for 6 hrs at 60° C., cooled to room temperature, poured into water (5 mL) and extracted with EtOAc (5 mL×2). The organic layers were combined, washed with water (5 mL) and brine (5 mL) successively, dried with anhydrous Na₂SO₄, and concentrated to obtain a residue which was purified with Pre-TLC (eluting with DCM/MeOH=10:1) to afford Compound 120-3 (350.0 mg, yield 88.4%) as a white solid. LCMS: 618.0, 620.0 ([M+H]⁺).

Compound 120-3 (290.0 mg, 0.4 mmol, 1.0 eq), INT C12 (349.0 mg, 0.7 mmol, 1.5 eq), K₃PO₄ (300.0 mg, 1.4 mmol, 3.0 eq), and cataCxium A Pd G₃ (52.0 mg, 0.07 mmol, 0.1 eq) were added successively into a mixed solution of THF and water (V_{THF}/V_{water}=5:1, 12 mL). The reaction mixture was purged with argon for 10 mins, stirred for 3 hrs at 60° C. under argon atmosphere, cooled to R.T, poured into water (15 mL) and extracted with DCM (15 mL×2). The organic layers were combined, washed with water (15 mL) and brine (15 mL) successively, dried with anhydrous Na₂SO₄, and concentrated to obtain a residue which was purified with Pre-TLC (eluting with DCM/MeOH=10:1) to afford a crude product containing Compound 120-4 (280.0 mg) as a yellow solid. LCMS: 906.1 ([M+H]⁺).

HCl/1,4-dioxane (3.5 mL) was added to a solution of the crude product containing Compound 120-4 (280.0 mg) in MeCN (5 mL) at R.T. The reaction mixture was stirred for 1 h at 30° C., and then concentrated to afford a crude product containing Compound 120-5 (250.0 mg) as a yellow solid. LCMS: 762.1 ([M+H]⁺).

CsF (500.0 mg, 3.3 mmol, 10.0 eq) was added to a solution of the crude product containing Compound 120-5 (250.0 mg) in DMF (3 mL) at R.T. The reaction mixture was stirred for 3 hrs at 60° C., and then filtered. The filtrate was purified with Prep-HPLC to afford Compound 120 (57.0 mg) as a white solid. ¹H NMR (400 MHz, DMSO-d₆): δ 10.07 (brs, 1H), 7.87-7.85 (m, 1H), 7.66 (s, 1H), 7.45-7.38 (m, 2H), 7.33 (s, 1H), 7.27-7.26 (m, 1H), 6.88 (d, J=2.4 Hz, 1H), 4.36-4.33 (m, 1H), 4.21-4.19 (m, 3H), 3.97 (s, 2H), 3.60-3.47 (m, 6H), 3.41 (s, 1H), 2.40-2.38 (m, 4H), 2.31 (s, 2H), 2.07 (s, 3H), 1.99-1.88 (m, 4H), 0.63-0.61 (m, 2H), 0.42-0.39 (m, 2H). LCMS: 606.3 ([M+H]⁺).

### Example 90

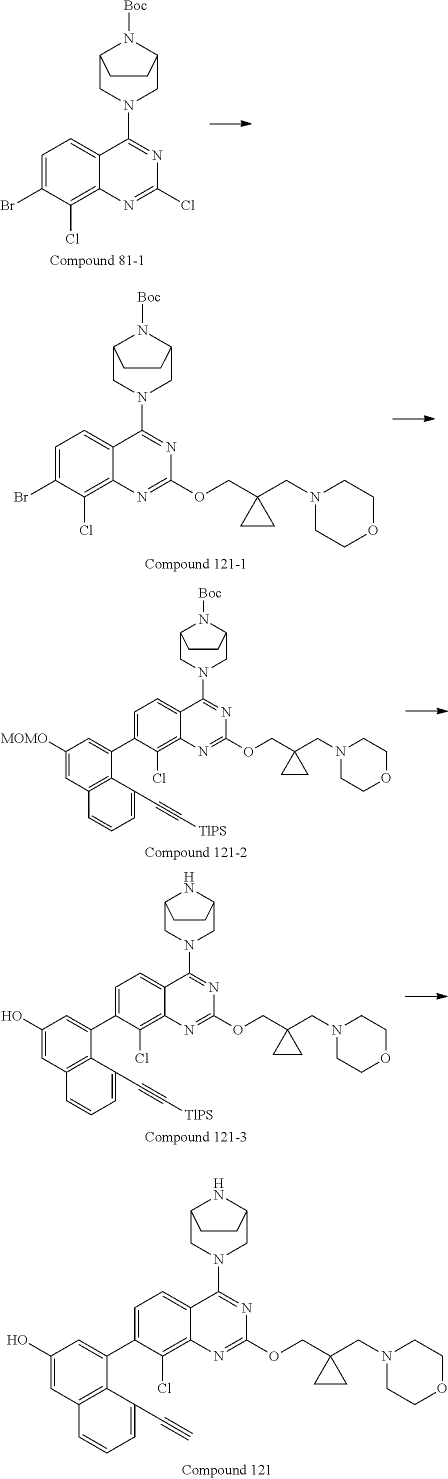

INT B2 (280.6 mg, 1.6 mmol, 2.0 eq), DABCO (44.9 mg, 0.4 mmol, 0.5 eq) and Cs₂CO₃ (782.0 mg, 2.4 mmol, 3.0 eq) were added to a mixture of Compound 81-1 (400 mg, 0.8 mmol, 1.0 eq) in THF and DMF (V_{THF}/V_{DMF}=1:1, 8 mL) at R.T. The reaction mixture was stirred at R.T for 16 hrs, poured into water (10 mL) and extracted with DCM (20 mL×2). The organic layers were combined, washed with brine (20 mL), dried over Na₂SO₄, and the filtrate was concentrated to obtain a residue. The residue was slurried with petroleum ether/EtOAc=2:1 and Compound 121-1 (270 mg, 52.8%) was collected by filtration as a white solid. LCMS: 622.1, 624.15 ([M+H]⁺).

Compound 121-1 (270 mg, 0.4 mmol, 1.0 eq), INT C12 (296.7 mg, 0.6 mmol, 1.5 eq), K₃PO₄ (254.7 mg, 1.2 mmol, 3.0 eq), CataCxium A Pd G₃ (43.7 mg, 0.06 mmol, 0.15 eq) were added successively into a mixed solution of THF and water (V_{THF}/V_{water}=1:1, 4 mL). The reaction mixture was purged with argon for 10 min, stirred for 2 hrs at 60° C. under argon atmosphere, cooled to R.T, poured into water (5 mL) and extracted with DCM (10 mL×2). The organic layers were combined, washed with water (10 mL) and brine (10 mL) successively, dried over Na₂SO₄, and concentrated to obtain a residue which was purified with Prep-TLC (eluting with EtOAc) to afford Compound 121-2 (100.0 mg, 25.3%) as a yellow solid. LCMS: 910 ([M+H]⁺).

HCl/1,4-dioxane (4.0M, 0.8 mL) was added to a solution of Compound 121-2 (100.0 mg, 0.1 mmol, 1.0 eq) in MeCN (4 mL) at 0° C. The reaction mixture was stirred at R.T for 1 h, and then concentrated under reduced pressure to afford a crude product containing Compound 121-3 (120.0 mg) as a yellow solid which was used for next step without further purification. LCMS: 766 ([M+H]⁺).

CsF (151.9 mg, 1.0 mmol, 10.0 eq) was added to a solution of the crude product containing Compound 121-3 (120.0 mg) in DMF (2 mL) at R.T. The reaction mixture was stirred for 3 hrs at 40° C., and then filtered. The filtrate was purified by Prep-HPLC to afford Compound 121 (13.0 mg, the yield of the above two steps is 19.4%) as a white solid. ¹H NMR (400 MHz, DMSO-d₆): δ 7.84-7.82 (m, 1H), 7.79-7.77 (m, 1H), 7.46-7.44 (m, 1H), 7.37-7.33 (m, 1H), 7.25-7.22 (m, 2H), 6.96 (d, J=2.8 Hz, 1H), 4.44-4.43 (m, 3H), 4.37-4.34 (m, 1H), 3.65-3.58 (m, 6H), 3.57-3.54 (m, 2H), 2.76 (s, 1H), 2.51-2.50 (m, 4H), 2.45-2.44 (m, 2H), 1.90-1.88 (m, 4H), 0.73-0.71 (m, 2H), 0.49-0.48 (m, 2H). LCMS: 610.0 ([M+H]⁺).

### Example 91

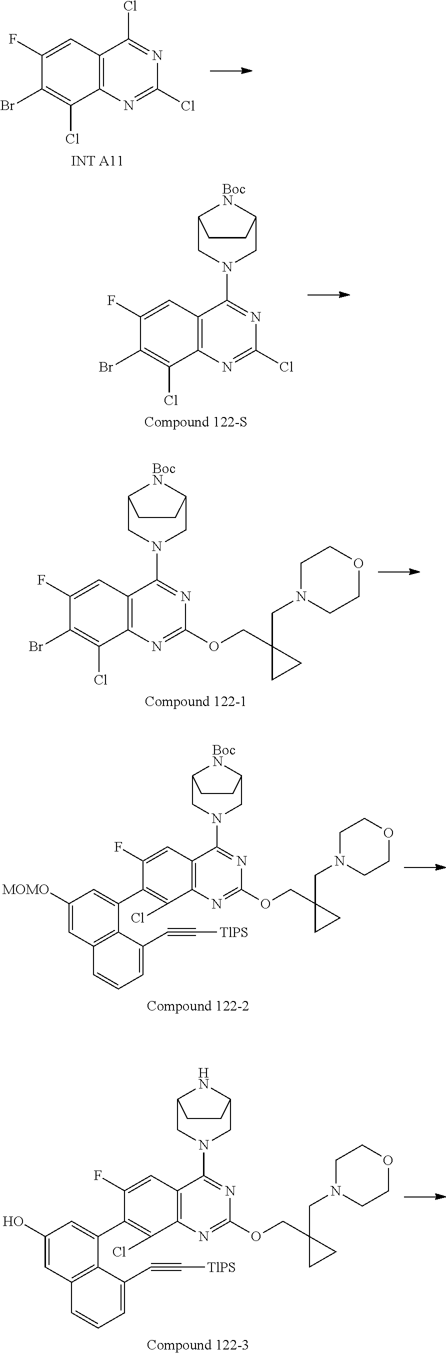

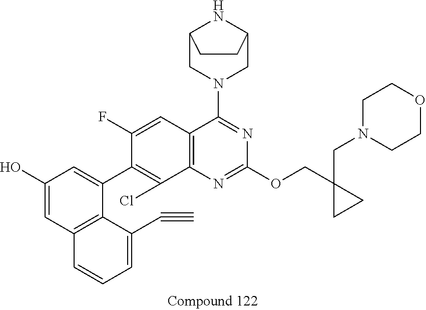 [SMILES: C#Cc1cccc2cc(O)cc(-c3c(F)cc4c(N5CC6CCC(C5)N6)nc(OCC5(CN6CCOCC6)CC5)nc4c3Cl)c12]

Compound 122-S was obtained following the procedure of Compound 120-2 was obtained following the procedure with INT A11 as start material.

A mixture of Compound 122-S (500 mg, 1.0 mmol, 1.0 eq), INT B2 (340 mg, 2.0 mmol, 2.0 eq), Cs₂CO₃ (980 mg, 3.0 mmol, 3.0 eq), DABCO (60 mg, 0.5 mmol, 0.5 eq) and a mixture solution of THF and DMF (V_{THF}/V_{DMF}=1/1, 10 mL) was stirred overnight at R.T, diluted with water (50 mL) after the reaction was completed, and extracted with EtOAc (50 mL×3). The organic layers were combined, washed with brine and dried over anhydrous Na₂SO₄, concentrated to obtain a residue which was purified with silica gel chromatography (petroleum ether/ethyl acetate=1/1) to afford Compound 122-1 (150 mg, 0.23 mol, 23% yield) as a yellow solid. ¹H NMR (300 MHz, CDCl₃) δ 7.43 (d, J=9.3 Hz, 1H), 4.42 (s, 2H), 4.34 (s, 2H), 4.27-4.16 (m, 2H), 3.67-3.59 (m, 4H), 3.59-3.48 (m, 2H), 2.46 (s, 4H), 2.40 (s, 2H), 2.00-1.91 (m, 2H), 1.85-1.75 (m, 2H), 1.51 (s, 9H), 0.75-0.65 (m, 2H), 0.50-0.40 (m, 2H). LCMS: 640.2 ([M+H]⁺).

A mixture of Compound 122-1 (100 mg, 0.16 mmol, 1.0 eq), INT C12 (225 mg, 0.47 mmol, 3.0 eq), Cs₂CO₃ (145 mg, 0.47 mmol, 3.0 eq), Cata Cxium A Pd G₃ (15 mg, 0.023 mmol, 0.15 eq), and a mixed solution toluene and H₂O (Vₜₒₗᵤₑₙₑ/V_{H2O}=4/1, 10 mL) was purged with argon, stirred at 100° C. for 4 hrs, diluted with water (30 mL) after the reaction was completed, and then extracted with EtOAc (3×30 mL). The organic layers were combined, washed with saturated brine and dried over anhydrous Na₂SO₄, and concentrated to obtain a residue which was purified with pre-TLC to afford Compound 122-2 (60 mg, 0.065 mmol, 40% yield) as a light-yellow solid. ¹H NMR (300 MHz, DMSO-d₆): δ 8.03 (dd, J=9.6, 1.5 Hz, 1H), 7.69-7.61 (m, 2H), 7.59-7.56 (m, 1H), 7.53-7.51 (m, 1H), 7.15 (d, J=2.7 Hz, 1H), 5.37 (d, J=1.2 Hz, 2H), 4.59-4.54 (m, 1H), 4.34-4.27 (m, 4H), 3.86-3.82 (m, 1H), 3.57-3.54 (m, 4H), 3.45 (s, 3H), 2.41-2.37 (m, 6H), 2.28-2.26 (m, 2H), 2.03-1.85 (m, 4H), 1.46 (s, 9H), 0.86-0.79 (m, 21H), 0.66-0.63 (m, 2H), 0.48-0.43 (m, 2H). LCMS: 928.4 ([M+H]⁺).

A solution of Compound 122-2 (60 mg, 0.065 mmol, 1.0 eq) and HCl/1,4-dioxane (4 M, 0.65 ml) in MeCN (3.2 mL) was stirred at 0° C. for 3.5 hrs, and then concentrated to afford a crude product containing Compound 122-3 as a yellow solid which was used directly to the next step without further purification. LCMS: 784.3 ([M+H]⁺).

A mixture of the crude product containing Compound 122-3, CsF (198 mg, 1.3 mmol, 20.0 eq) and DMF (4.0 ml) was stirred overnight at 40° C., and then purified with pre-HPLC to afford Compound 122 (16 mg, 0.026 mmol, 39% yield) as a white solid. ¹H NMR (400 MHz, DMSO-d₆) δ 10.15 (s, 1H), 7.88 (dd, J=8.0, 1.6 Hz, 1H), 7.63 (d, J=10.1 Hz, 1H), 7.43 (dt, J=15.1, 6.3 Hz, 2H), 7.33 (d, J=2.5 Hz, 1H), 6.97 (d, J=2.5 Hz, 1H), 4.37-4.10 (m, 4H), 3.62-3.41 (m, 9H), 2.39 (s, 4H), 2.33-2.27 (m, 2H), 1.76-1.57 (m, 4H), 0.65-0.63 (m, 2H), 0.41-0.39 (m, 2H). LCMS: 628.2 ([M+H]⁺).

### Example 92

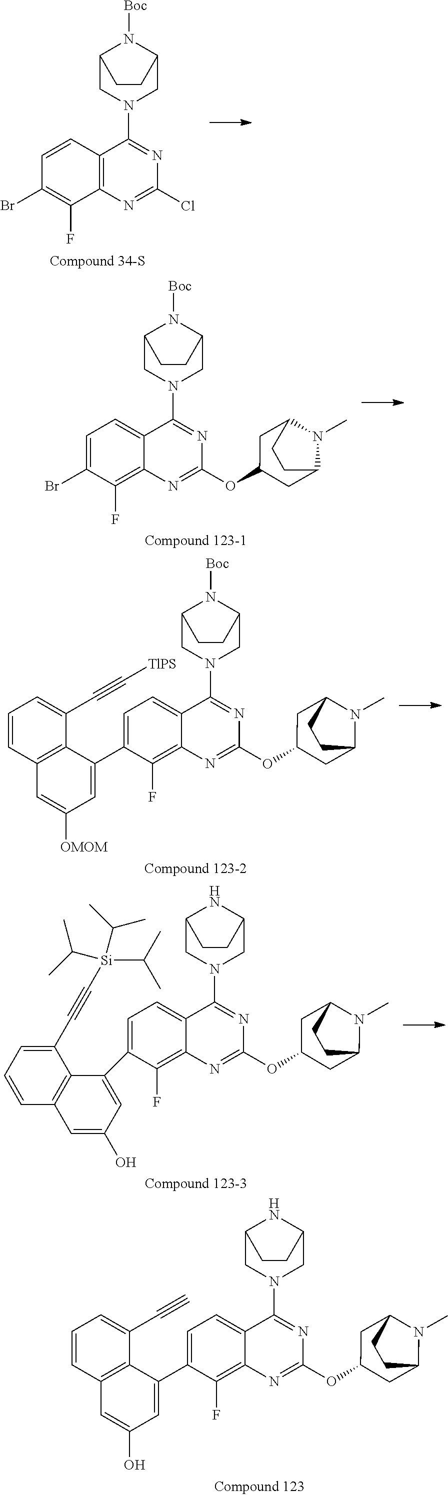

Compound 123 was obtained following the procedure of Compound 34 with Compound 34-S as start material.

### Example 93

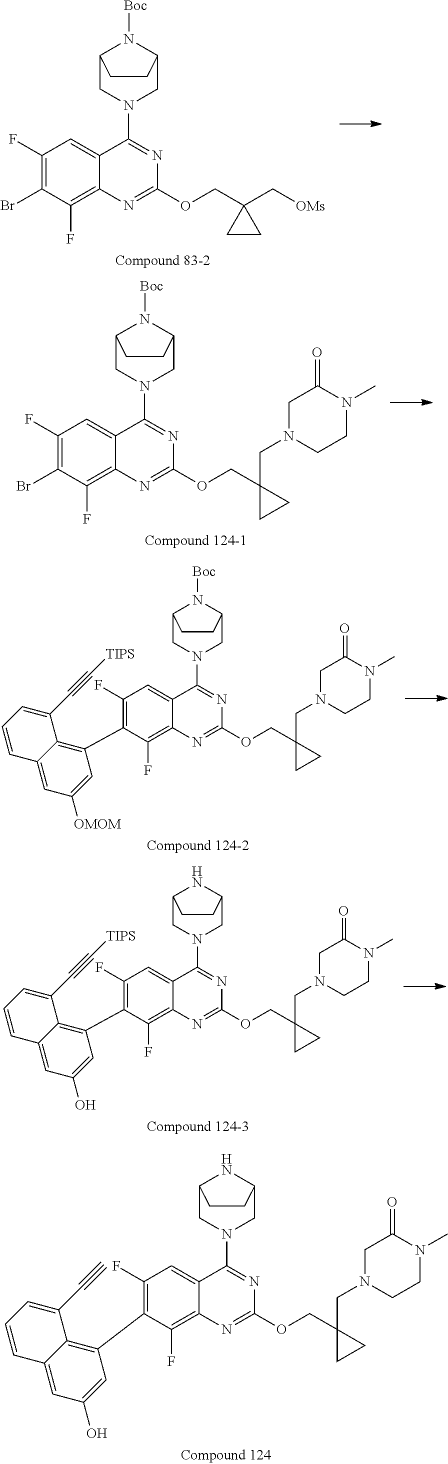

A mixture of Compound 83-2 (300 mg, 0.47 mmol), 1-Methylpiperazin-2-one (102 mg, 0.89 mmol), TEA (149 mg, 1.47 mmol) and DMF (5 mL) was stirred at 80° C. for 18 hrs, and then water (30 mL) was added. The reaction mixture was extracted with EA (40 mL). The organic layer was washed with brine, and concentrated under reduced pressure to obtain a residue which was purified with Pre-TLC (EA) to afford Compound 124-1 (247 mg, 0.38 mmol). MS: m/z 651 [M+1]⁺.

A mixture of Compound 124-1 (247 mg, 0.38 mmol), INT C12 (216 mg, 0.44 mmol), cataCXium A Pd G₃ (27 mg, 0.037 mmol), Cs₂CO₃ (375 mg, 1.15 mmol), toluene (8 mL) and water (2 mL) was purged with nitrogen, stirred at 100° C. for 16 hrs. The reaction mixture was concentrated under reduced pressure to obtain a residue. The residue was diluted with EA (40 mL) and water (30 mL), and the organic layer was concentrated under reduced pressure to obtain a crude product which was purified with Pre-TLC (Hex:EA=0:1, v/v) to afford Compound 124-2 (265 mg, 0.28 mmol). MS: m/z 939 [M+H]⁺.

A solution of Compound 124-2 (265 mg, 0.28 mmol), HCl/1, 4-dioxane (4 M, 2 mL) in CH₃CN (5 mL) was stirred at R.T for 1 h, and then concentrated under reduced pressure to obtain a residue. The pH of the mixture of the residue, EA (40 mL) and water (30 mL) was adjusted to 8-9 with aq. NaHCO₃ (5%). The organic phase was washed with brine, dried over anhydrous Na₂SO₄, and concentrated under reduced pressure to afford Compound 124-3 (314.3 mg, 0.40 mmol). MS: m/z 795 [M+H]⁺.

A mixture of Compound 124-3 (314.3 mg, 0.40 mmol), CsF (972 mg, 6.40 mmol) and DMF (5 mL) was stirred at R.T for 16 hrs, and then concentrated under reduced pressure to obtain a residue which was purified with Pre-HPLC (C18 column; eluent A: 0.1% TFA in water; eluent B: CH₃CN; Gradient: 15% B to 40% B in 35 min at a flow rate of 60 mL/min; 245 nm) to afford Compound 124 (260.9 mg, 0.30 mmol). MS: m/z 639 [M+H]⁺.

### Example 94

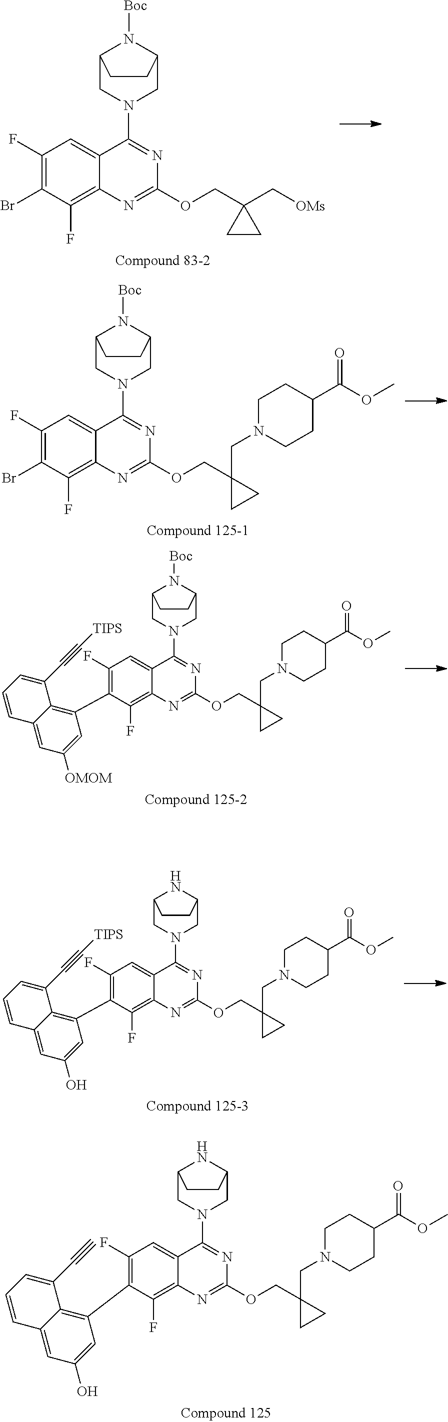

Compound 125 was obtained following the procedure of Compound 83 with Compound 83-2 as start material.

### Example 95

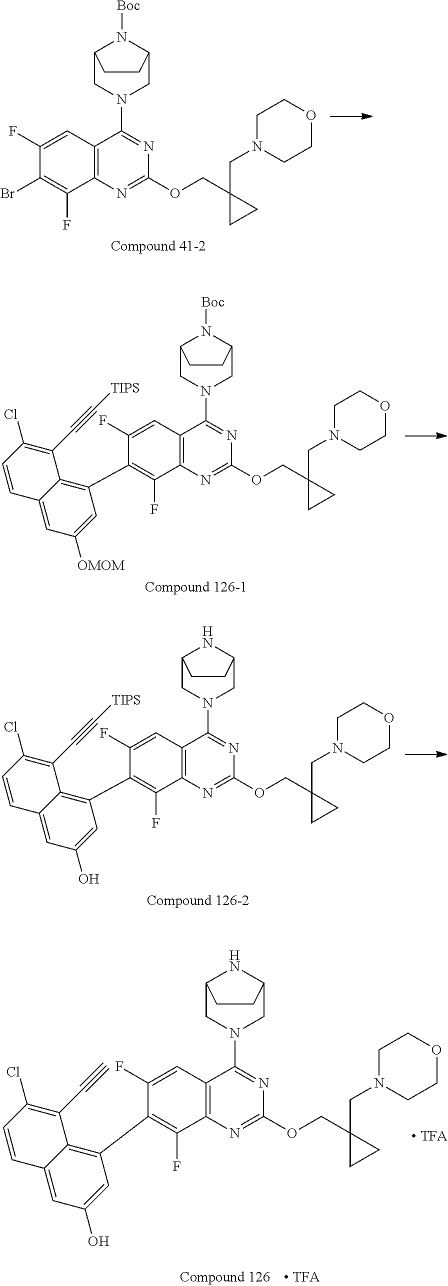

Cs₂CO₃ (132 mg, 0.41 mmol) and cataCXium A Pd G₃ (25 mg, 0.03 mmol) were added to a solution of Compound 41-2 (149 mg, 0.24 mmol) and INT C23 (116 mg, 0.22 mmol) in toluene (4 mL) and water (1 mL). The reaction mixture was purged with nitrogen, stirred at 100° C. for 3 hrs, cooled to R.T, and then filtered. The filtrate was concentrated under reduced pressure to obtain a residue which was purified with prep-TLC (EA:Hex=1:1, v/v) to afford Compound 126-1 (35 mg, 15.5%). LCMS m/z: 946 [M+H]⁺.

A solution of Compound 126-1 (35 mg, 0.04 mmol) and HCl/1,4-dioxane (4 M, 1 mL) in 1,4-dioxane (2 mL) was stirred for 2.5 hrs at R.T, and then concentrated under reduced pressure to afford a crude product containing Compound 126-2 which was used in next step without further purification. LCMS m/z: 802 [M+H]⁺.

A mixture of the crude product containing Compound 126-2, CsF (0.56 g, 3.69 mmol) and DMF (2 mL) was stirred at R.T for 3 hrs, then filtered. The filtrate was concentrated under reduced pressure to obtain a residue which was purified with Prep-HPLC (Venusil PrepG C18, 30*250 mm, 10 μm; eluent A: 0.1% TFA in water; eluent B: CH₃CN; Gradient: 15% eluent B to 40% eluent B in 35 min at a flow rate of 60 mL/min; 240 nm) to afford the TFA salt of Compound 126 (8.9 mg, 16.2%) as a yellow solid. LCMS m/z: 646 [M+H]⁺.

### Example 96

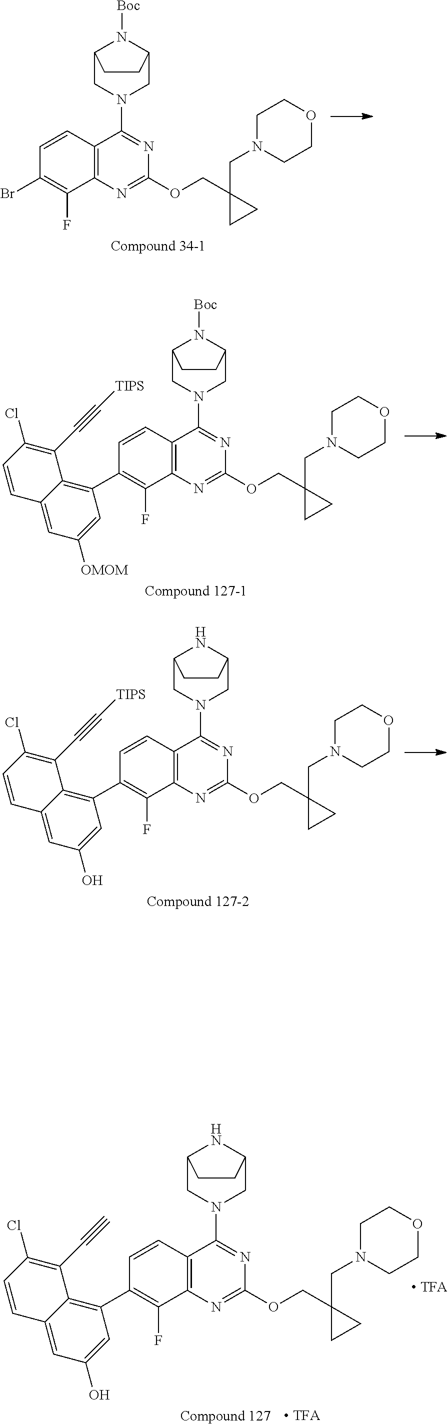

Cs₂CO₃ (278 mg, 0.85 mmol) and cataCXium A Pd G₃ (43 mg, 0.06 mmol) were added to a solution of Compound 34-1 (269 mg, 0.44 mmol) and INT C23 (353 mg, 0.67 mmol) in toluene (4 mL) and water (1 mL). The reaction mixture was purged with nitrogen, stirred at 100° C. for 4 hrs, cooled to R.T, and then EA (10 mL) and water (10 mL) were added. The organic layer was concentrated under reduced pressure to obtain a residue which was purified with prep-TLC (EA:Hex=1:1, v/v) to afford Compound 127-1 (102 mg, 25.5%). LCMS m/z: 928 [M+H]⁺.

A solution of Compound 127-1 (102 mg, 0.10 mmol) and HCl/1,4-dioxane (4 M, 2 mL) in 1,4-dioxane (2 mL) was stirred for 4 hrs at R.T, and then concentrated under reduced pressure to obtain a residue. The residue was diluted with EA (10 mL) and water (10 mL). The pH of the resulting mixture was adjusted to 9 with NaHCO₃. The organic phase was separated and concentrated under reduced pressure to afford a crude product containing Compound 127-2 which was used in next step without further purification. LCMS m/z: 784 [M+H]⁺.

A mixture of the crude product containing Compound 127-2, CsF (0.35 g, 2.30 mmol) and DMF (2 mL) was stirred for 16 hrs at R.T, then filtered. The filtrate was concentrated under reduced pressure to obtain a residue which was purified with Prep-HPLC (Daisogel-C18 column, 50*250 mm, 10 μm; eluent A: 0.1% TFA in water; eluent B: CH₃CN; Gradient: 20% eluent B to 40% eluent B in 35 min at a flow rate of 60 mL/min; 240 nm) to afford the TFA salt of Compound 127 (24.1 mg, 14.8%) as a yellow solid. LCMS m/z: 628 [M+H]⁺.

### Example 97

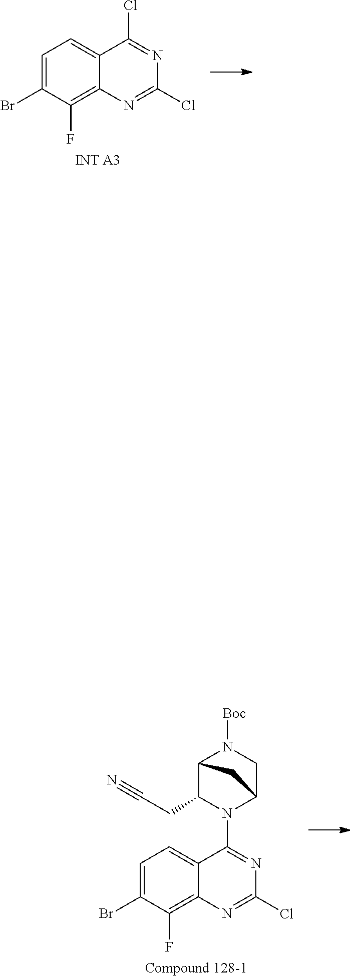

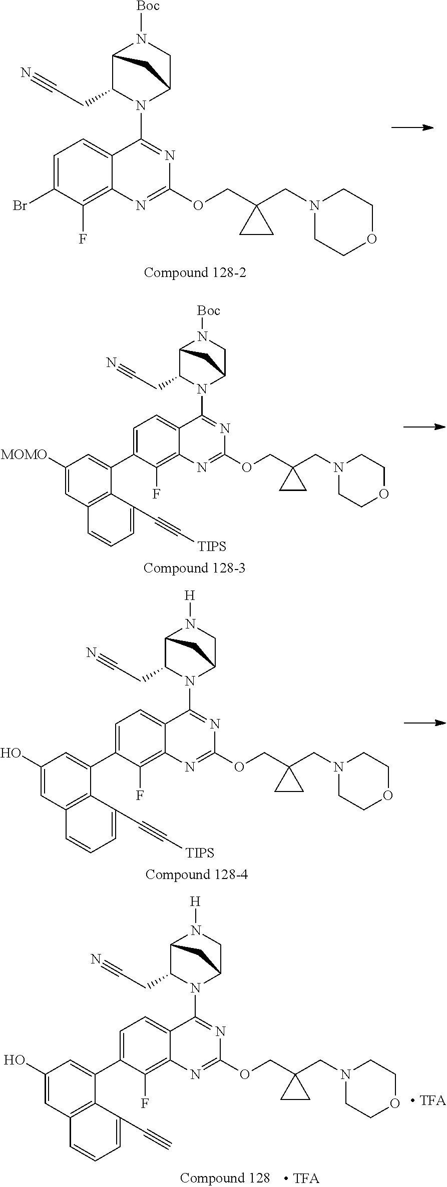

DIEA (162 mg, 1.25 mmol) was added to a solution of INT A3 (186 mg, 0.63 mmol) and INT D1 (221 mg, 0.96 mmol) in DCM (5 mL) at R.T. The reaction mixture was stirred for 4 hrs, and then concentrated under reduced pressure to obtain a residue. The residue was slurred with EA (2 mL) and Hex (5 mL), and a crude product containing Compound 128-1 was collected by filtration as a white solid (371 mg). LCMS m/z: 496 [M+H]⁺.

Triethylenediamine (42 mg, 0.37 mmol) and Cs₂CO₃ (511 mg, 1.57 mmol) were added to a solution of Compound 128-1 (371 mg, 0.75 mmol) and INT B2 (243 mg, 1.42 mmol) in THF (2 mL) and DMF (2 mL) at R.T. The reaction mixture was stirred for 22.5 hrs at R.T, and then water (15 mL) was added. The resulting mixture was extracted with EA (2×15 mL), and the organic layers were combined and concentrated under reduced pressure to obtain a residue which was purified with prep-TLC (EA:Hex=1.5:1, v/v) to afford Compound 128-2 (88 mg, 18.7%). LCMS m/z: 631 [M+H]⁺.

Cs₂CO₃ (90 mg, 0.28 mmol) and cataCXium A Pd G₃ (10 mg, 0.01 mmol) were added to a solution of Compound 128-2 (88 mg, 0.14 mmol) and INT C12 (100 mg, 0.20 mmol) in toluene (2 mL) and water (0.5 mL). The reaction mixture was purged with N₂, stirred at 100° C. for 6.5 hrs, cooled to R.T, diluted with water (10 mL), and then extracted with EA (2×10 mL). The organic layers were combined and concentrated under reduced pressure to obtain a residue which was purified with prep-TLC (EA:Hex=1:1, v/v) to afford Compound 128-3 (96 mg, 75.0%). LCMS m/z: 919 [M+H]⁺.

A solution of Compound 128-3 (96 mg, 0.10 mmol) and HCl/1,4-dioxane (4 M, 2 mL) in 1,4-dioxane (2 mL) was stirred for 2 hrs at R.T, then concentrated under reduced pressure to obtain a residue. The pH of a mixture of the residue and water (5 mL) was adjusted to 9 with NaHCO₃. The resulting mixture was extracted with EA (2×10 mL). The organic layers were combined and concentrated under reduced pressure to afford a crude product containing Compound 128-4 (80 mg). LCMS m/z: 775 [M+H]⁺.

CsF (0.18 g, 1.19 mmol) was added to a solution of the crude product containing Compound 128-4 (80 mg) in DMF (3 mL). The reaction mixture was stirred at R.T for 24 hrs, then filtered. The filtrate was concentrated under reduced pressure to obtain a residue which was purified with Prep-HPLC (Daisogel-C18 column, 50*250 mm, 10 μm; eluent A: 0.1% TFA in water; eluent B: CH₃CN; Gradient: 15% eluent B to 40% eluent B in 35 min at a flow rate of 60 mL/min; 230 nm) to afford the TFA salt of Compound 128 (31.1 mg, 20.6%) as a yellow solid. LCMS m/z: 619 [M+H]⁺.

### Example 98

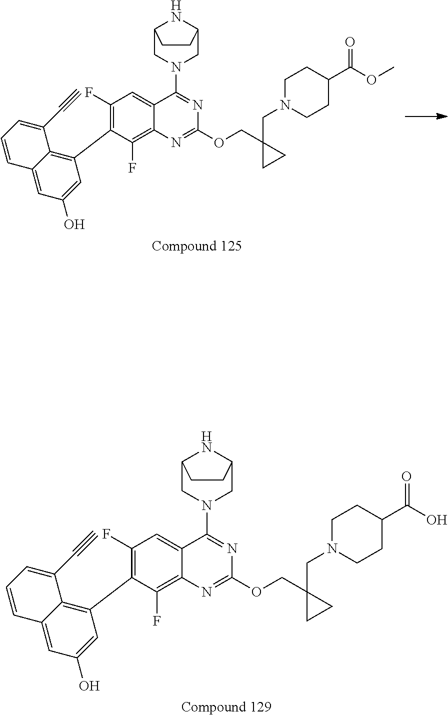 [SMILES: C#Cc1cccc2cc(O)cc(-c3c(F)cc4c(N5CC6CCC(C5)N6)nc(OCC5(CN6CCC(C(=O)OC)CC6)CC5)nc4c3F)c12 ; C#Cc1cccc2cc(O)cc(-c3c(F)cc4c(N5CC6CCC(C5)N6)nc(OCC5(CN6CCC(C(=O)O)CC6)CC5)nc4c3F)c12]

Compound 129 was obtained hydrolysis of Compound 125 with NaOH.

### Example 99

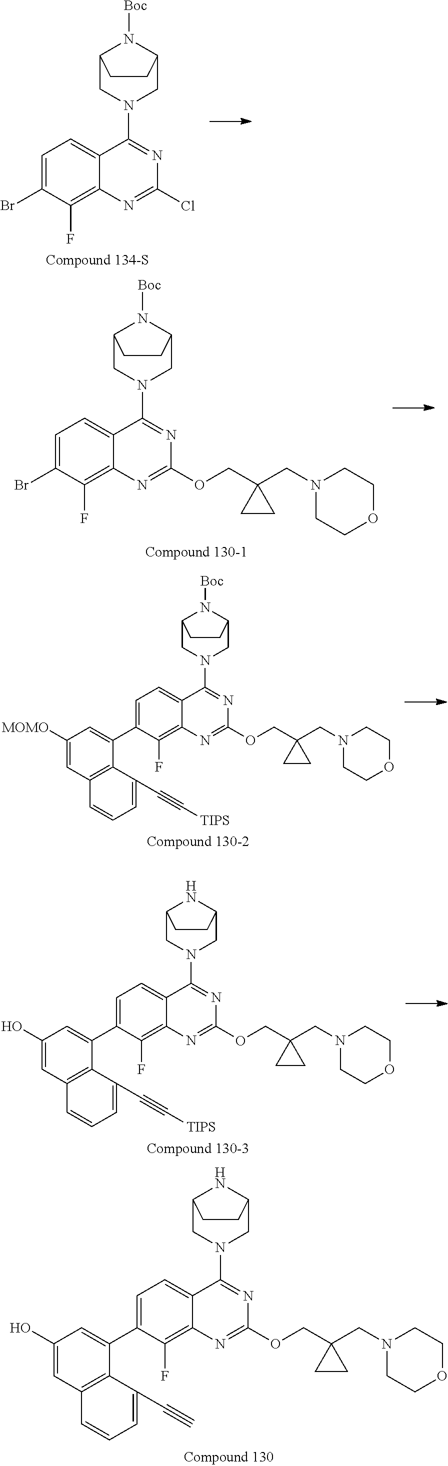

Compound 34-S (510 mg, 1.08 mmol, 1.0 eq), INT B18 (400 mg, 2.16 mmol, 2.0 eq), DABCO (121 mg, 1.08 mmol, 1.0 eq), and Cs₂CO₃ (1.06 g, 3.23 mmol, 3.0 eq) were added successively into a mixed solution of THF and DMF (V_{THF}/V_{DMF}=1:1, 10 mL). The reaction mixture was stirred at R.T for 5 hrs, poured into water (10 mL) and extracted with EtOAc (10 mL×2). The organic layers were combined, washed with brine (10 mL), dried over anhydrous Na₂SO₄, and concentrated to obtain a residue which was purified with silica gel chromatography (eluting with petroleum ether/EtOAc=5:1) to afford Compound 130-1 (150 mg, yield 19.3%) as yellow oil. LCMS m/z: 620[M+H]⁺.

A mixture of Compound 130-1 (150 mg, 0.24 mmol, 1.0 eq), INT C12 (362 mg, 0.73 mmol, 3.0 eq), K₃PO₄ (154 mg, 0.73 mmol, 3.0 eq), CataCxium A Pd G₃ (24.6 mg, 0.036 mmol, 0.15 eq), and a mixed solution of THF and water (V_{THF}/V_{water}=5:1, 2 mL) was purged with argon for 10 mins, stirred at 60° C. for 4 hrs under argon atmosphere, cooled to R.T, poured into water (5 mL) and extracted with EtOAc (5 mL×2). The organic layers were combined, washed with brine (5 mL), dried with anhydrous Na₂SO₄, and concentrated to obtain a residue which was purified with Pre-TLC (eluting with ether/EtOAc=3:1) to afford Compound 130-2 (68 mg, yield 31.0%) as yellow oil. LCMS m/z: 908[M+H]⁺.

A solution of Compound 130-2 (68 mg, 0.074 mmol, 1.0 eq) and HCl/Dioxane (4 M, 0.6 mL) in MeCN (2 mL) was stirred at R.T for 1 h, then concentrated under reduced pressure to afford a crude product containing Compound 130-3 (75 mg) as a yellow solid. LCMS m/z: 764[M+H]⁺.

A mixture of the crude product containing Compound 130-3 (75 mg), CsF (223 mg, 1.0 mmol, 10.0 eq) and DMF (1 mL) was stirred overnight at R.T, and then filtered. The filtrate was purified with Prep-HPLC to afford Compound 130 (4.3 mg, yield 26.2%) as a white solid. ¹H NMR (400 MHz, CD₃OD-d₄): δ 7.80-7.74 (m, 2H), 7.48 (d, J=6.8 Hz, 1H), 7.37 (t, J=7.6 Hz, 1H), 7.28-7.24 (m, 2H), 7.04 (d, J=2.4 Hz, 1H), 4.64-4.52 (m, 4H), 3.93-3.91 (m, 2H), 3.61-3.58 (m, 2H), 3.31-3.29 (m, 4H), 2.88 (s, 1H), 2.59 (s, 2H), 2.47-2.46 (m, 4H), 2.08-1.93 (m, 10H). LCMS: 608.10 ([M+H]⁺).

### Example 100

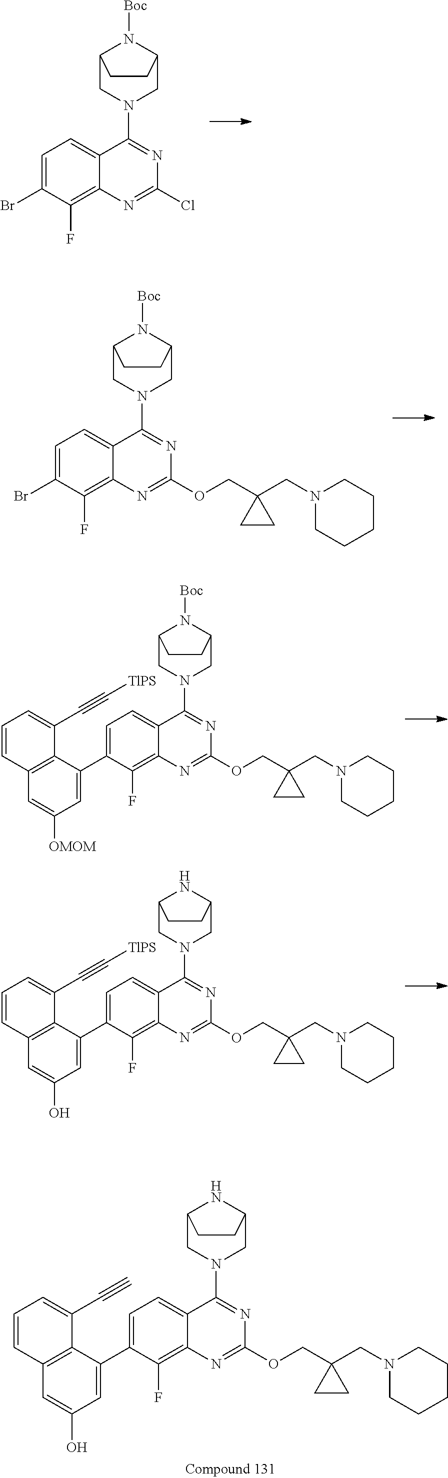

The Compound 131 was synthesized according to the synthetic procedure of Example 3.

### Example 101

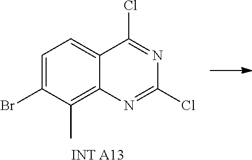

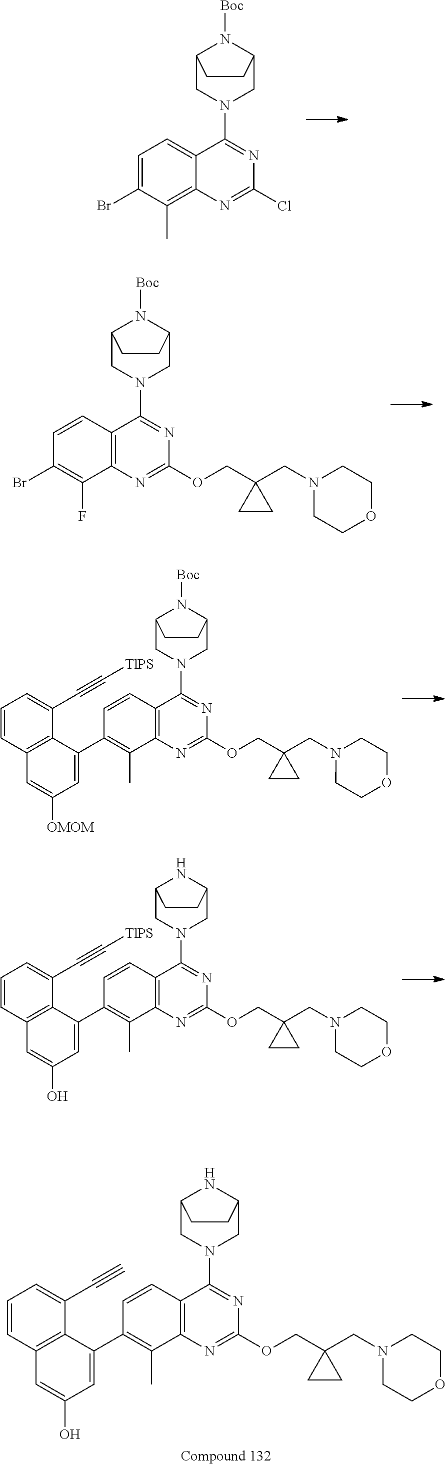

The Compound 132 was synthesized according to the synthetic procedure of Example 3.

### Example 102

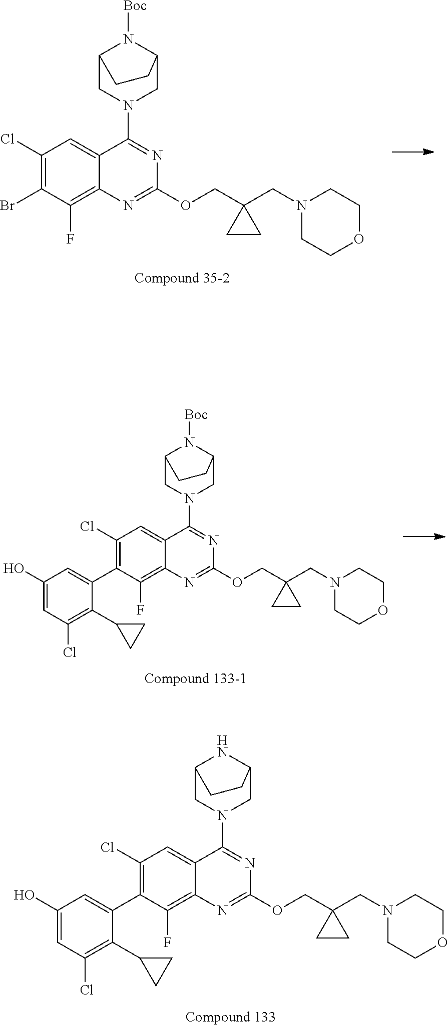 [SMILES: CC(C)(C)OC(=O)N1C2CCC1CN(c1nc(OCC3(CN4CCOCC4)CC3)nc3c(F)c(Br)c(Cl)cc13)C2 ; CC(C)(C)OC(=O)N1C2CCC1CN(c1nc(OCC3(CN4CCOCC4)CC3)nc3c(F)c(-c4cc(O)cc(Cl)c4C4CC4)c(Cl)cc13)C2 ; Oc1cc(Cl)c(C2CC2)c(-c2c(Cl)cc3c(N4CC5CCC(C4)N5)nc(OCC4(CN5CCOCC5)CC4)nc3c2F)c1]

Compound 35-2 (142 mg, 0.2 mmol, 1.0 eq), INT C24 (85 mg, 0.3 mmol, 1.5 eq), K₃PO₄ (141 mg, 0.6 mmol, 3.0 eq), CxiumA-Pd G3 (25 mg, 0.03 mmol, 0.15 eq) were added successively into a mixed solution of THF and water (V_{THF}/V_{water}=5:1, 3 mL). The reaction mixture was purged with argon for 10 mins, stirred for 3 hrs at 60° C. under argon atmosphere, cooled to R.T, poured into water (5 mL) and extracted with EtOAc (5 mL×2). The organic layers were combined, washed with water (5 mL) and brine (5 mL) successively, dried with anhydrous Na₂SO₄, and concentrated to obtain a residue which purified with Pre-TLC (eluting with DCM/MeOH=10:1) to afford Compound 133-1 (70 mg, yield 43.4%) as a gray solid. LCMS: m/z=728.3 [M+H]⁺.

TFA (1 mL) was added to a solution of Compound 133-1 (70 mg, 0.1 mmol, 1.0 eq) in DCM (5 mL) at R.T. the reaction mixture was stirred for 1 h, and then concentrated to obtain a residue which was purified with Prep-HPLC to afford Compound 133 (16 mg, yield 19.5%) as a white solid. ¹H NMR (400 MHz, DMSO-d₆): δ 7.95 (s, 1H), 6.97 (d, J=2.4 Hz, 1H), 6.54 (d, J=2.4 Hz, 1H), 4.52-4.84 (m, 1H), 4.42-4.39 (m, 1H), 4.37-4.26 (m, 2H), 4.21-4.18 (m, 2H), 4.03-4.00 (m, 2H), 3.83-3.79 (m, 1H), 3.75-3.69 (m, 3H), 3.63-3.61 (m, 2H), 3.28-3.26 (m, 2H), 3.14-3.12 (m, 2H), 1.97-1.96 (m, 4H), 1.67-1.62 (m, 1H), 0.87-0.79 (m, 4H), 0.59-0.51 (m, 2H), 0.18-0.13 (m, 2H). LCMS: m/z=628.2 ([M+H−2TFA]⁺).

### Example 103

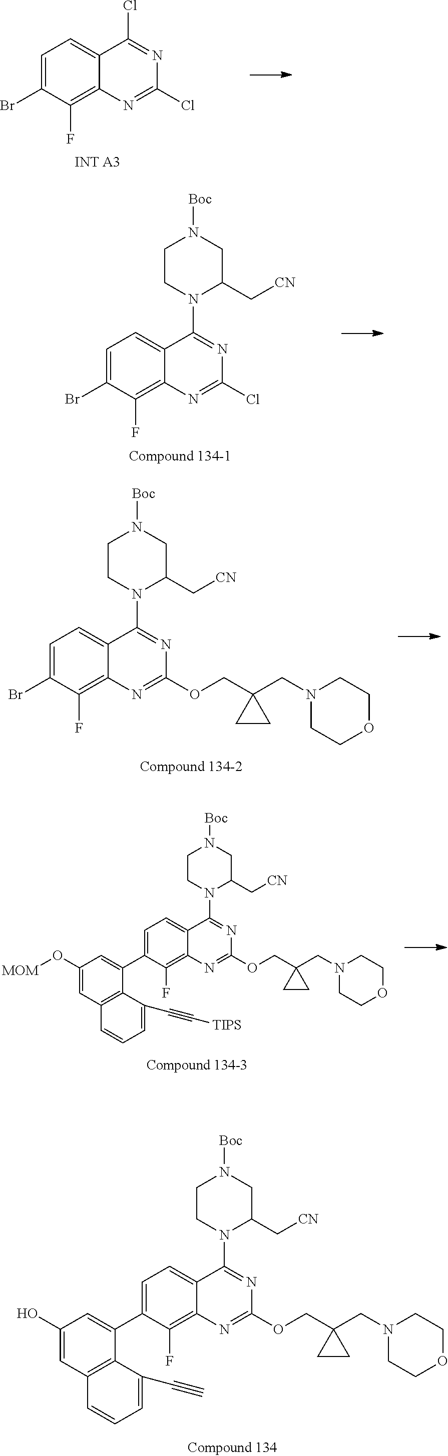

A solution of INT A3 (0.48 g, 1.62 mmol), DIEA (1.47 g, 11.37 mmol) and tert-butyl 3-(cyanomethyl)piperazine-1-carboxylate (826 mg, 3.67 mmol) in DCM (10 mL) was stirred at 80° C. for 2.5 hrs, and then concentrated under reduced pressure to obtain a residue. The residue was purified by Prep-TLC (EtOAc:PE=1:3, v/v) to afford Compound 134-1 (557 mg, 1.15 mmol, 70.84%). MS m/z: 484 [M+H]⁺.

A mixture of Compound 134-1 (557 mg, 1.15 mmol), INT B2 (305 mg, 1.78 mmol), DABCO (102 mg, 0.91 mmol) and Cs₂CO₃ (1002 mg, 3.08 mmol), DMF (5 mL) and THF (5 mL) was stirred at room temperature for 40 hrs, diluted with water (20 mL) and then extracted with EA (20 mL). The organic layers were combined, dried over Na₂SO₄ and concentrated under reduced pressure to obtain a residue which was purified by Pre-TLC to afford Compound 134-2 (0.09 g, 0.15 mmol, 12.64%). MS m/z: 619 [M+H]⁺.

Cs₂CO₃ (30 mg, 0.092 mmol) and cataCXium A Pd G₃ (11 mg, 0.015 mmol) were added to a solution of Compound 134-2 (30 mg, 0.048 mmol) and INT C12 (32 mg, 0.065 mmol) in toluene (4 mL) and water (1 mL). The reaction mixture was stirred at 100° C. for 2.5 hrs under nitrogen atmosphere, and then concentrated under reduced pressure to obtain a residue which was purified by Pre-TLC (EA:Hex=1:1, v/v) to afford Compound 134-3 (40 mg, 91.05%). MS m/z: 907 [M+H]⁺.

A solution of Compound 134-3 (40 mg, 0.044 mmol) and HCl/1,4-dioxane (1 mL, 4 mol/L in 1,4-dioxane) in acetonitrile (3 mL) was stirred at R.T for 30 mins, and then concentrated under reduced pressure to obtain a residue. The residue dissolved in DMF (5 mL) and CsF (0.61 g, 4.02 mmol) was added. The reaction mixture was stirred overnight at R.T, and then filtered to obtain a filtrate which was concentrated under reduced pressure to obtain a residue. The residue was purified by Pre-HPLC to afford the TFA salt of Compound 134 (2.6 mg, 0.0036 mmol). MS m/z: 607 [M+H]⁺.

### Example 104

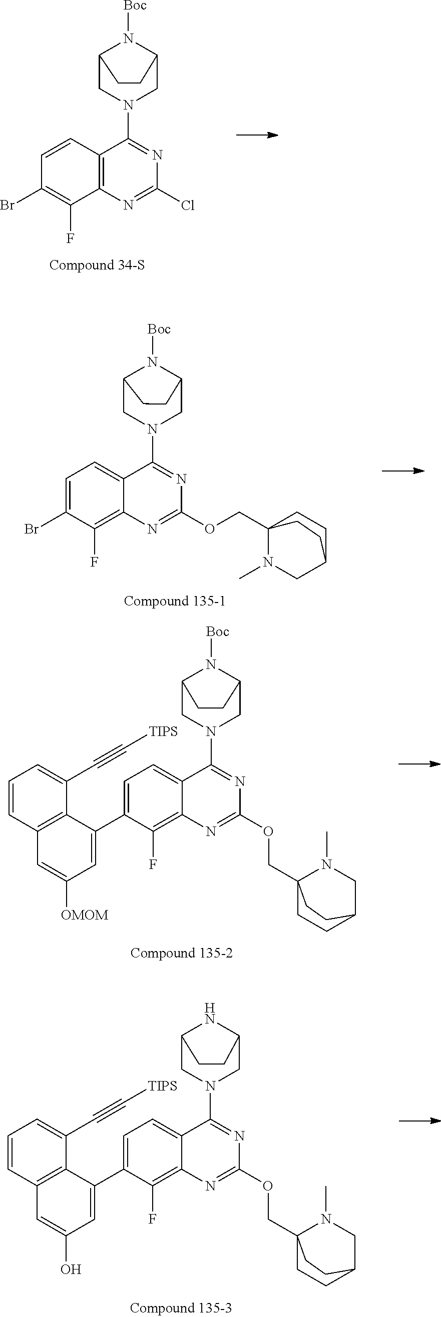

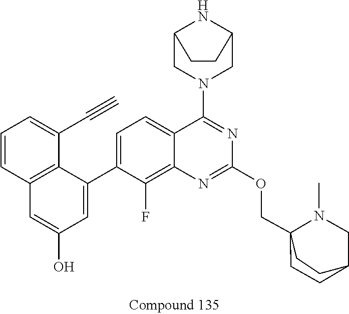 [SMILES: C#Cc1cccc2cc(O)cc(-c3ccc4c(N5CC6CCC(C5)N6)nc(OCC56CCC(CC5)CN6C)nc4c3F)c12]

Compound 135 was obtained following the procedure of Compound 34 with Compound 34-S as start material.

### Example 105

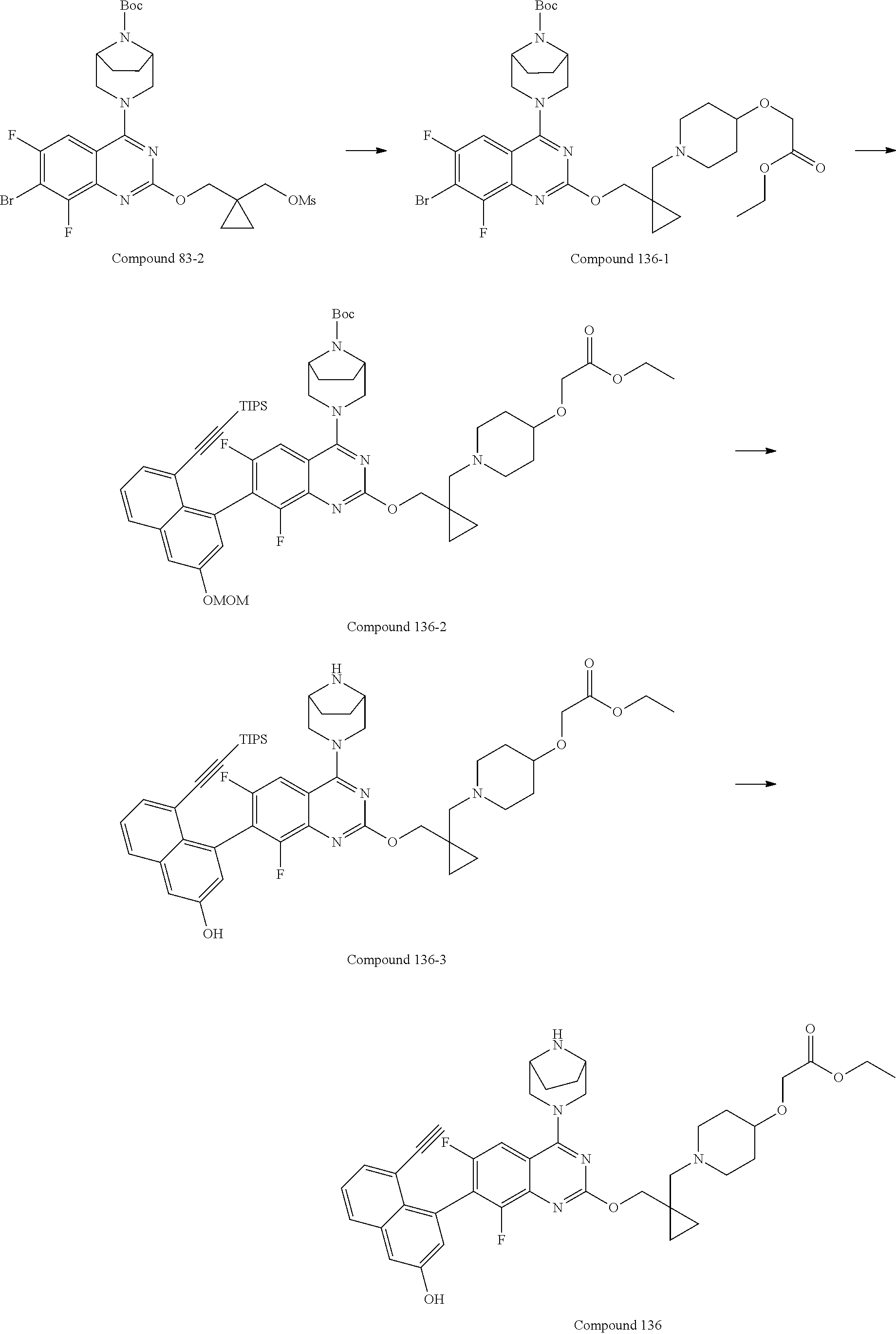

Compound 136 was obtained following the procedure of Compound 83 with Compound 83-2 as start material.

The compounds in the following table can be synthesized with reference to the above examples.

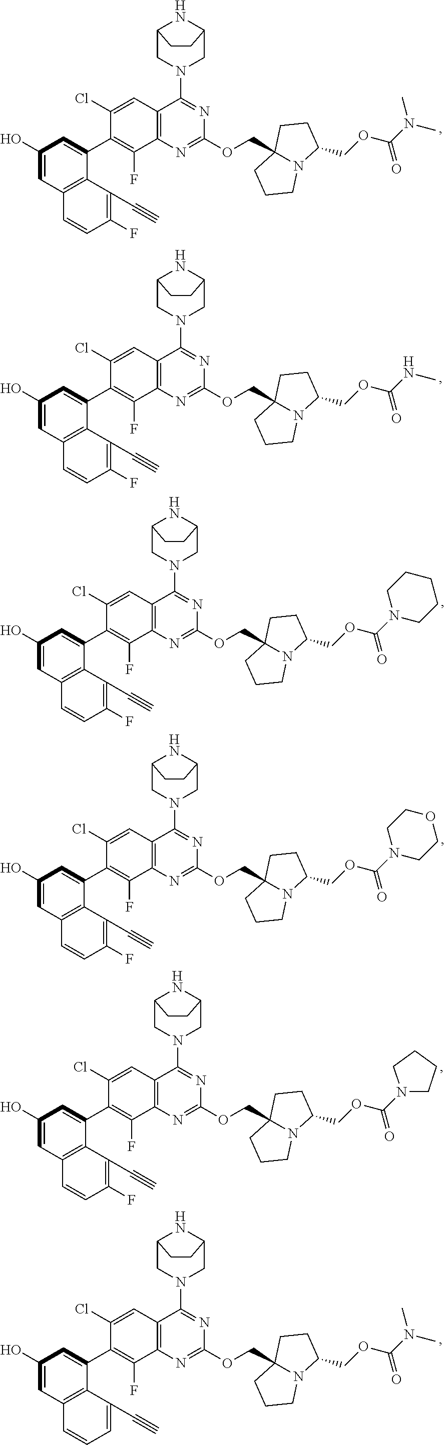

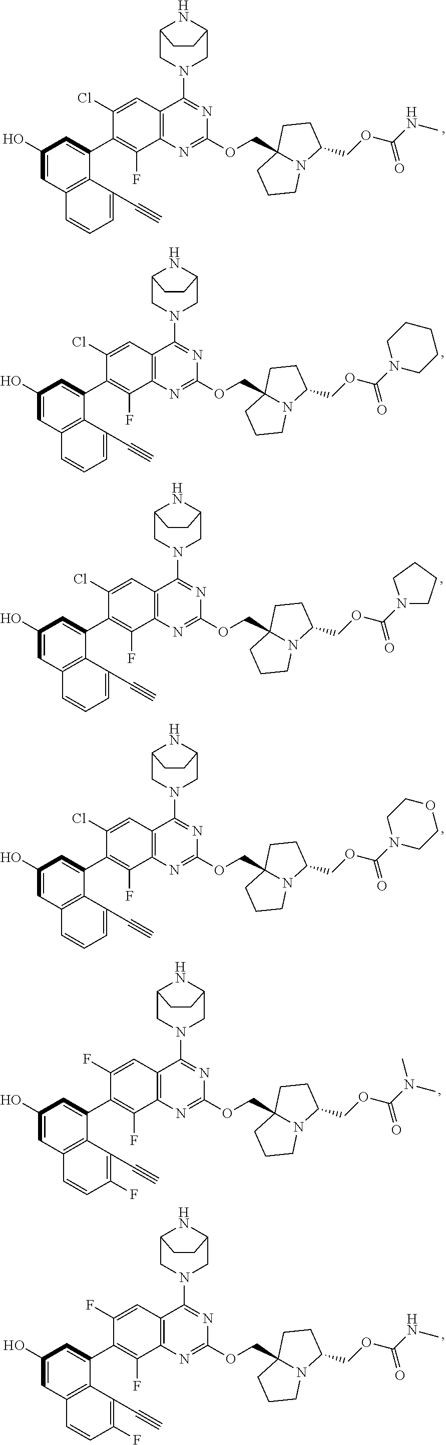

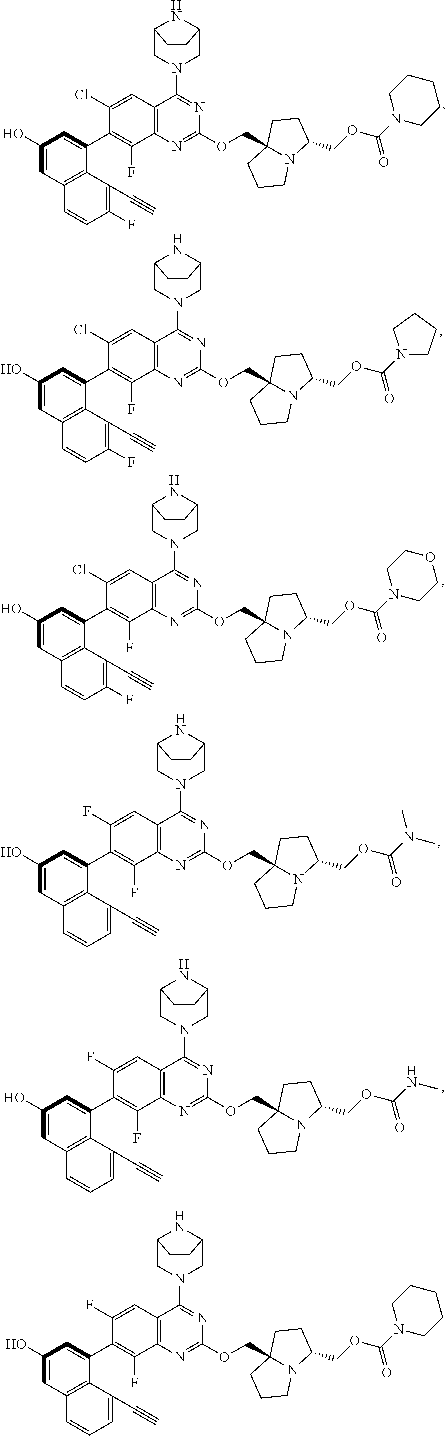

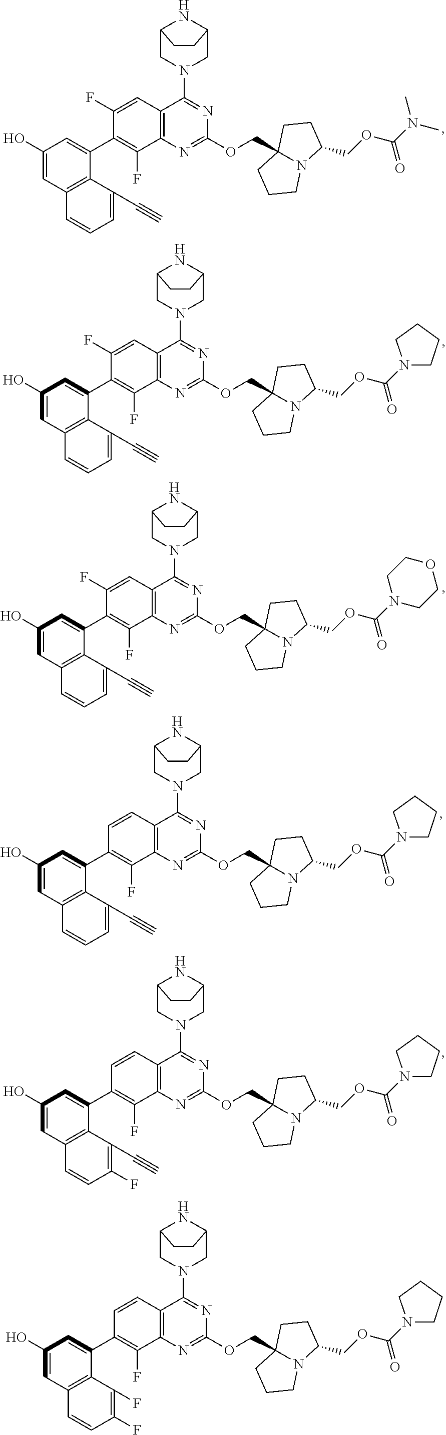

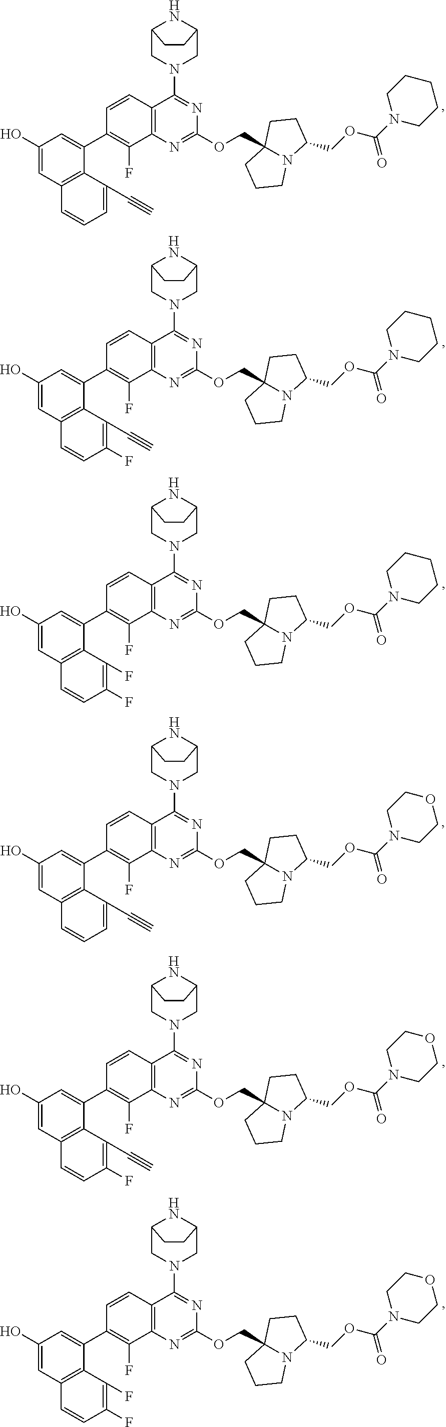

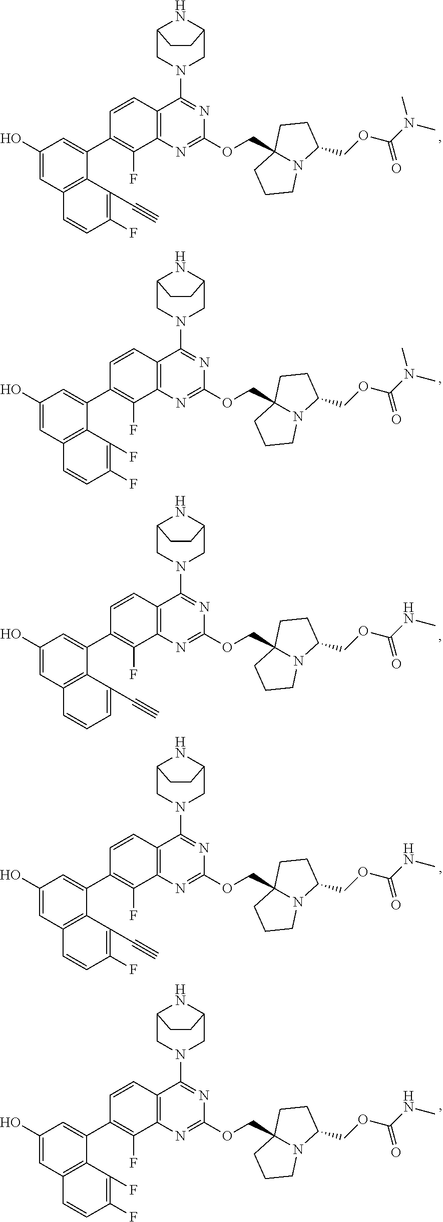 [SMILES: C#Cc1c(F)ccc2cc(O)cc(-c3ccc4c(N5CC6CCC(C5)N6)nc(OC[C@]56CCCN5[C@@H](COC(=O)N(C)C)CC6)nc4c3F)c12 ; CN(C)C(=O)OC[C@H]1CC[C@@]2(COc3nc(N4CC5CCC(C4)N5)c4ccc(-c5cc(O)cc6ccc(F)c(F)c56)c(F)c4n3)CCCN12 ; C#Cc1cccc2cc(O)cc(-c3ccc4c(N5CC6CCC(C5)N6)nc(OC[C@]56CCCN5[C@@H](COC(=O)NC)CC6)nc4c3F)c12 ; C#Cc1c(F)ccc2cc(O)cc(-c3ccc4c(N5CC6CCC(C5)N6)nc(OC[C@]56CCCN5[C@@H](COC(=O)NC)CC6)nc4c3F)c12 ; CNC(=O)OC[C@H]1CC[C@@]2(COc3nc(N4CC5CCC(C4)N5)c4ccc(-c5cc(O)cc6ccc(F)c(F)c56)c(F)c4n3)CCCN12]

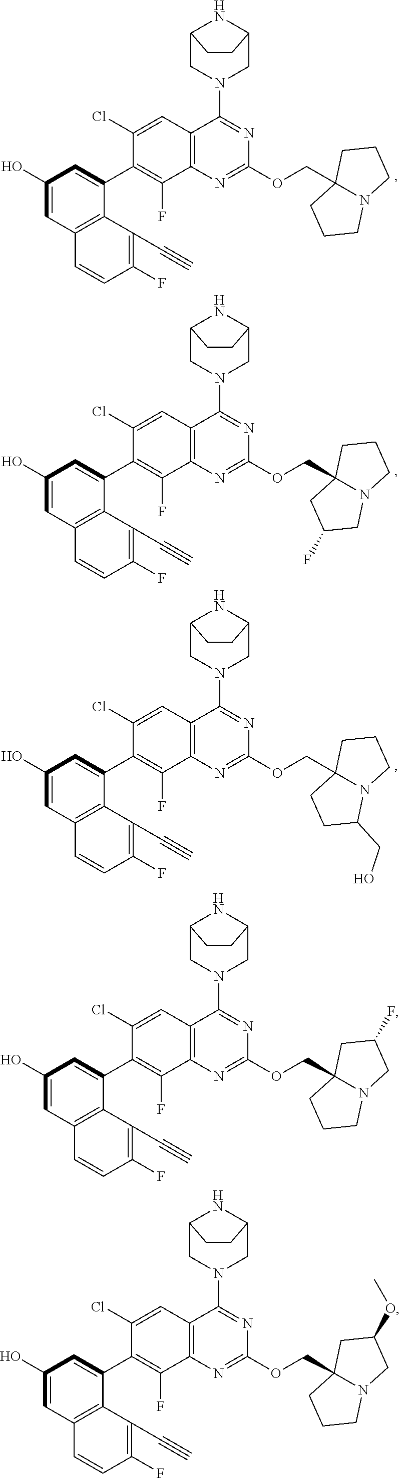

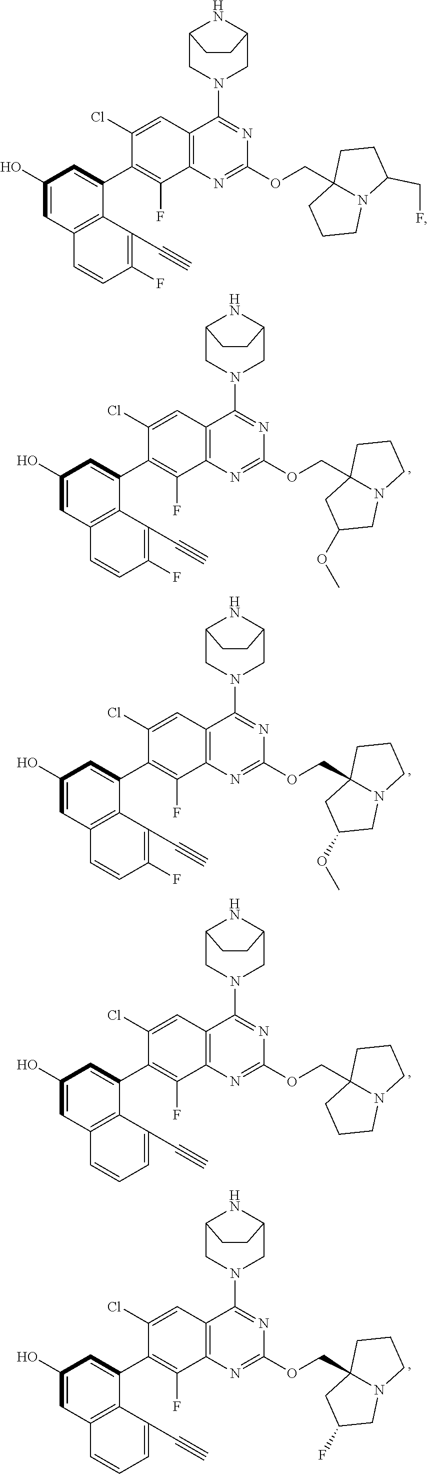

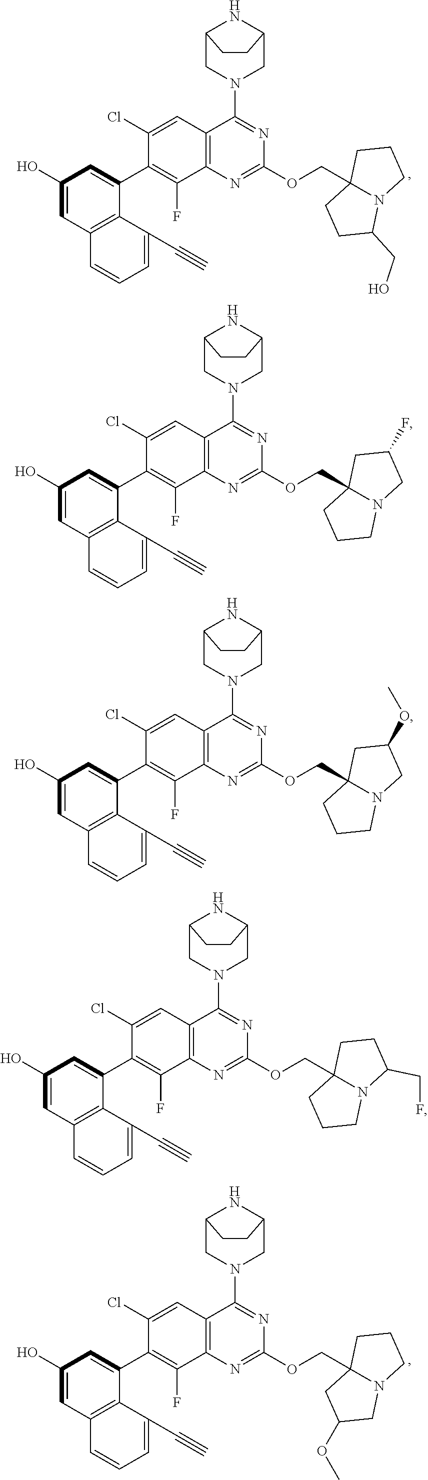

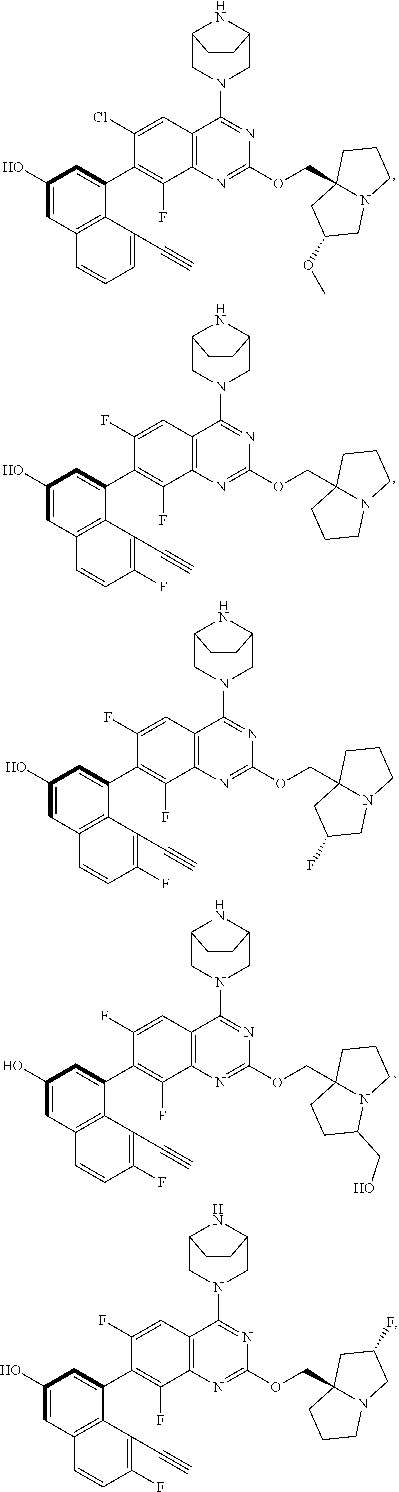

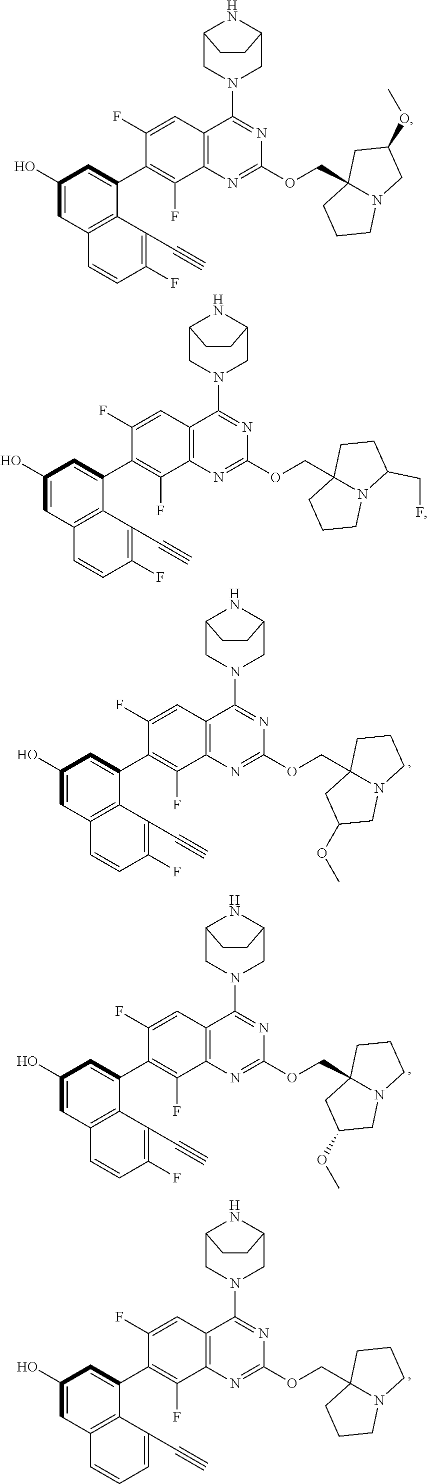

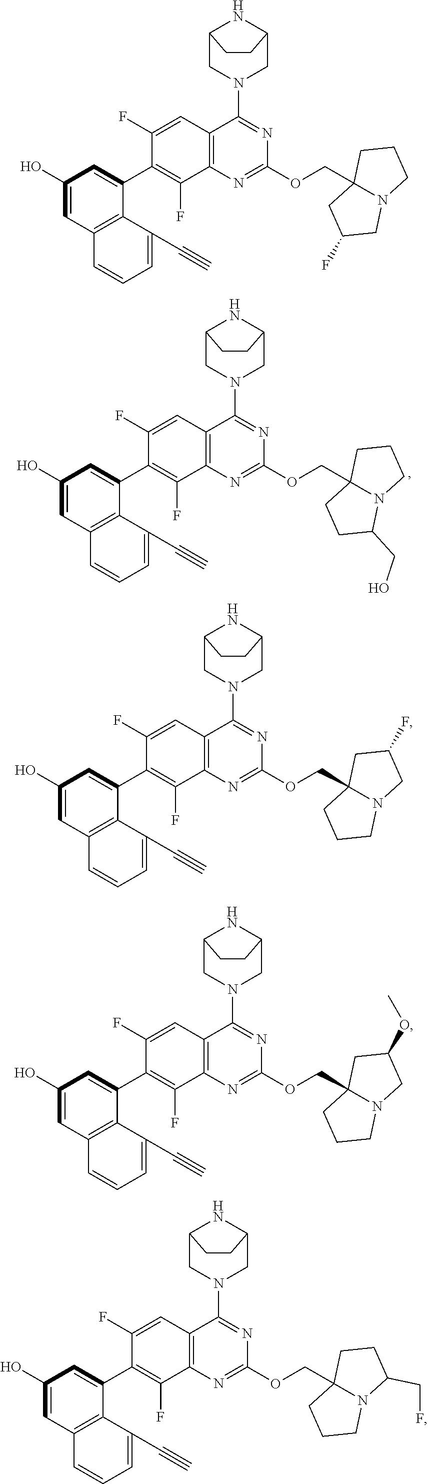

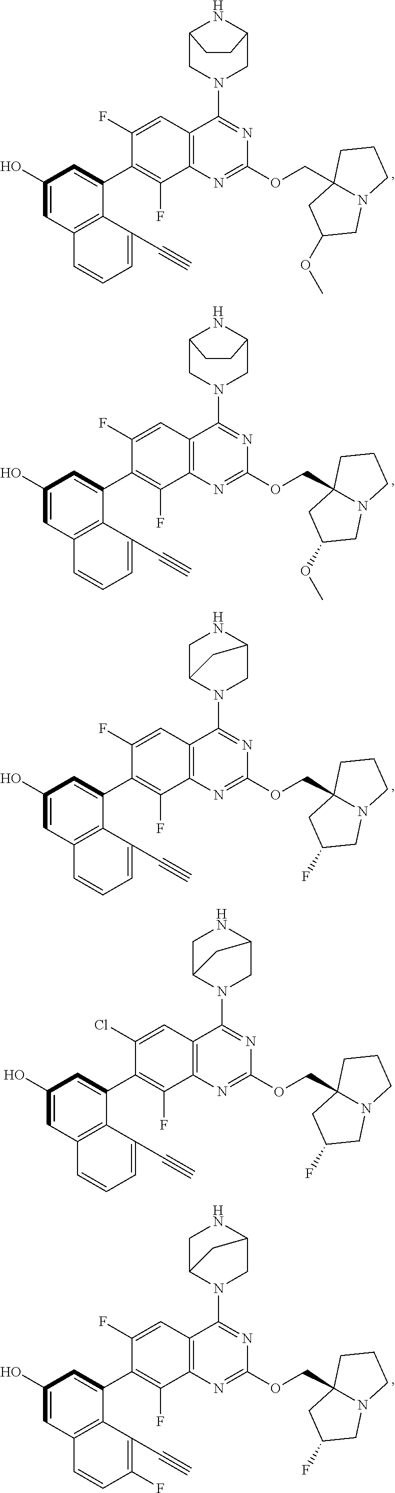

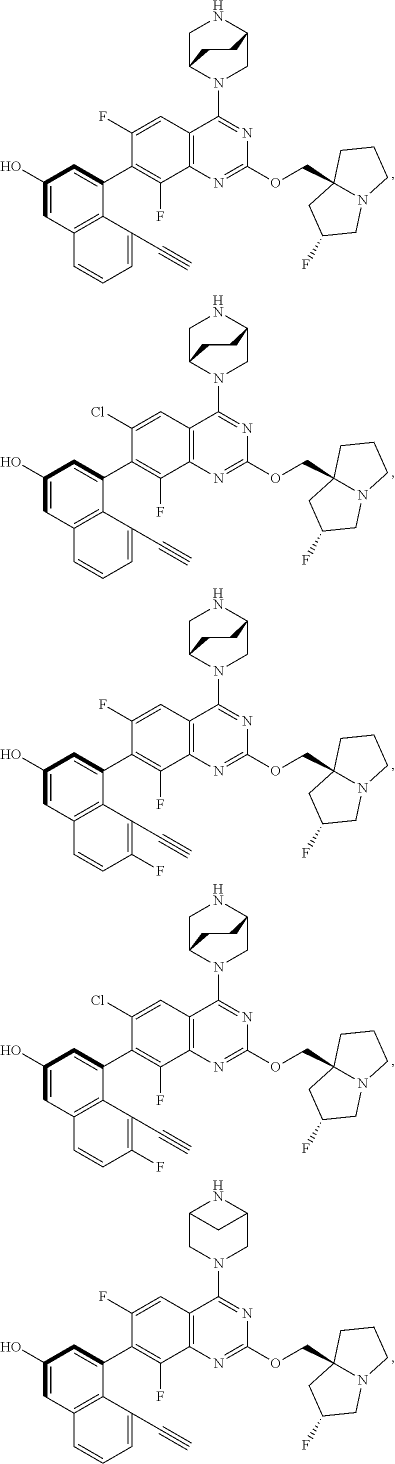

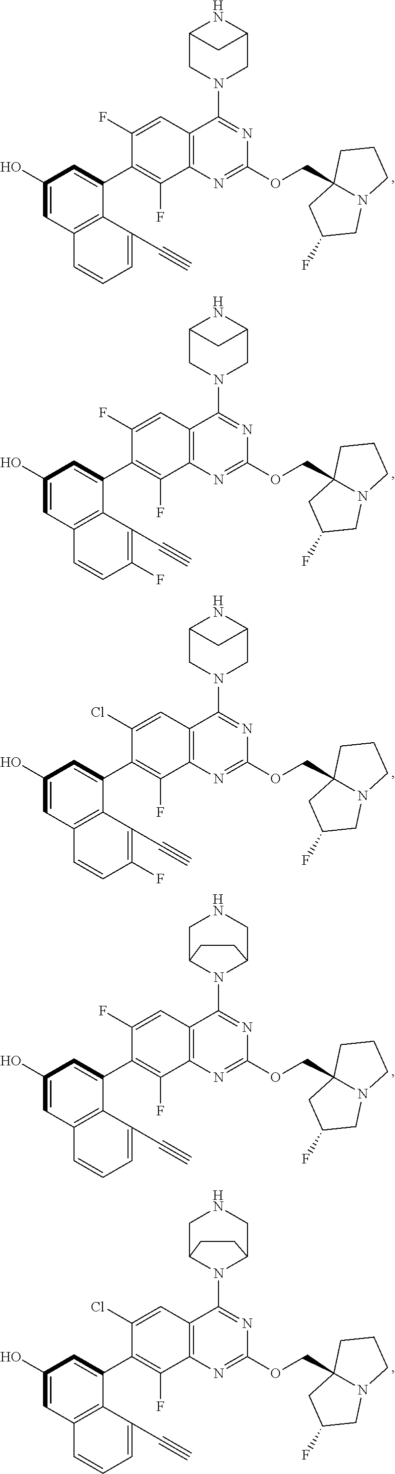

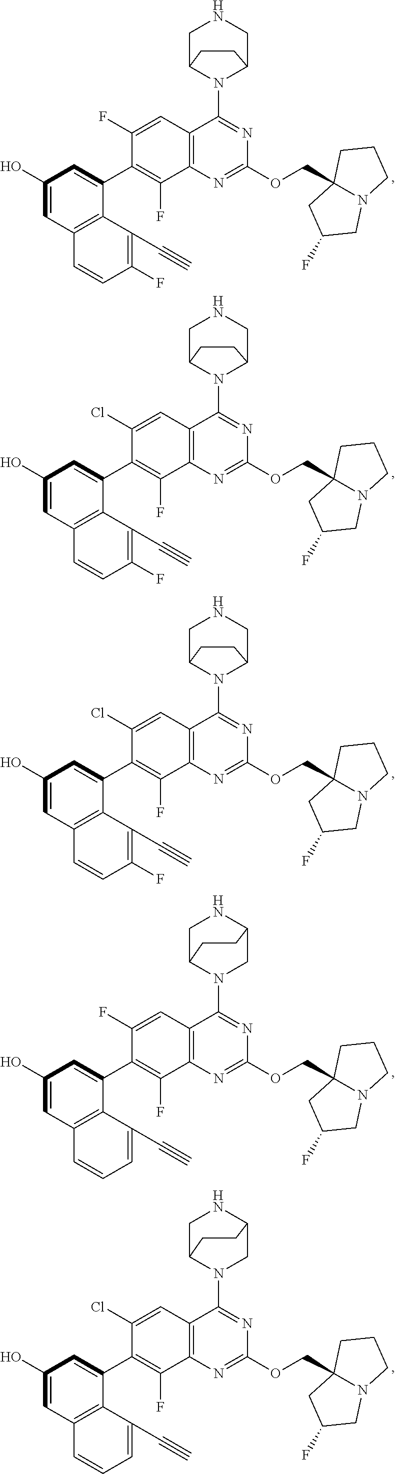

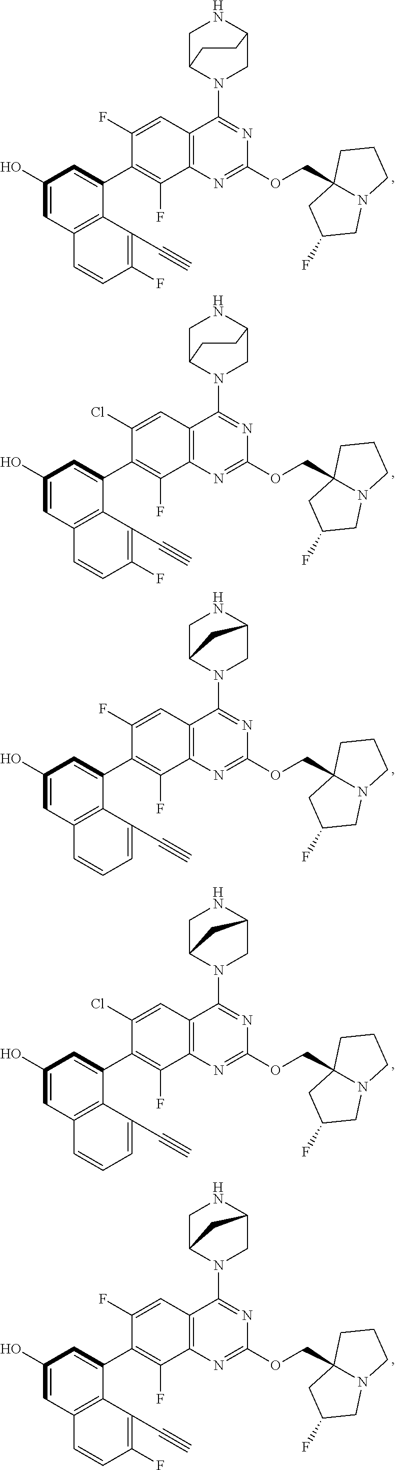

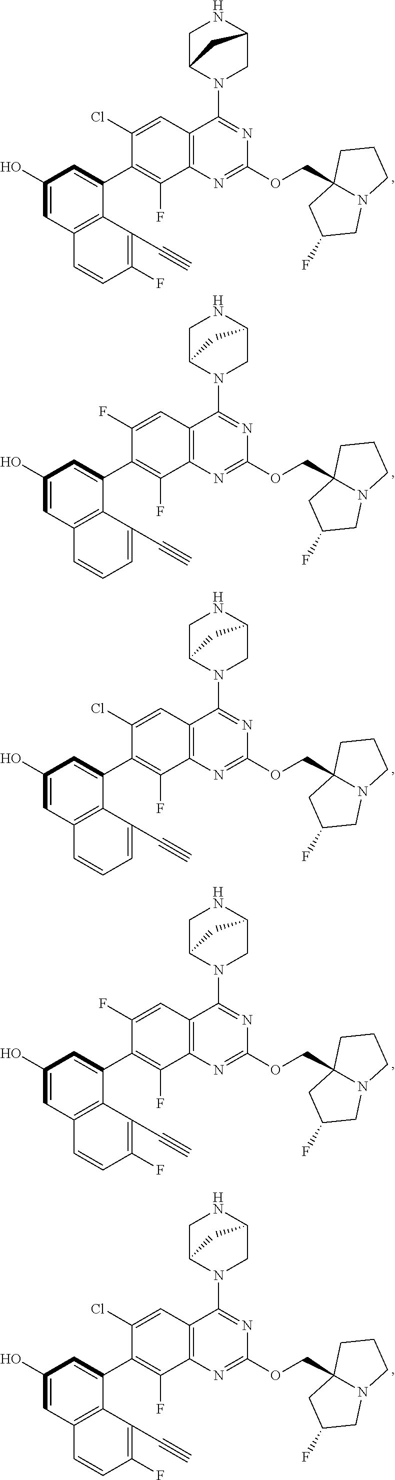

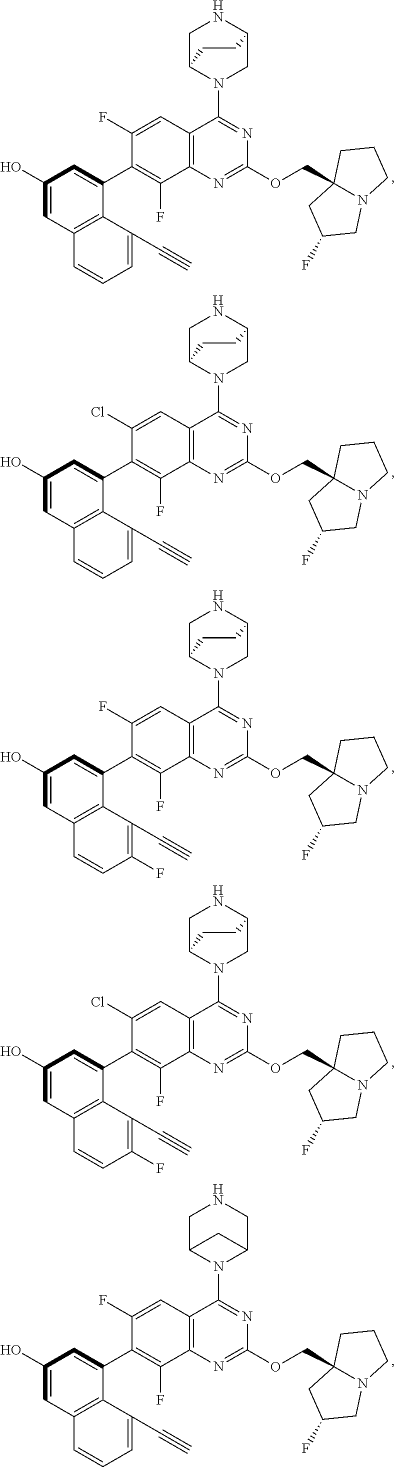

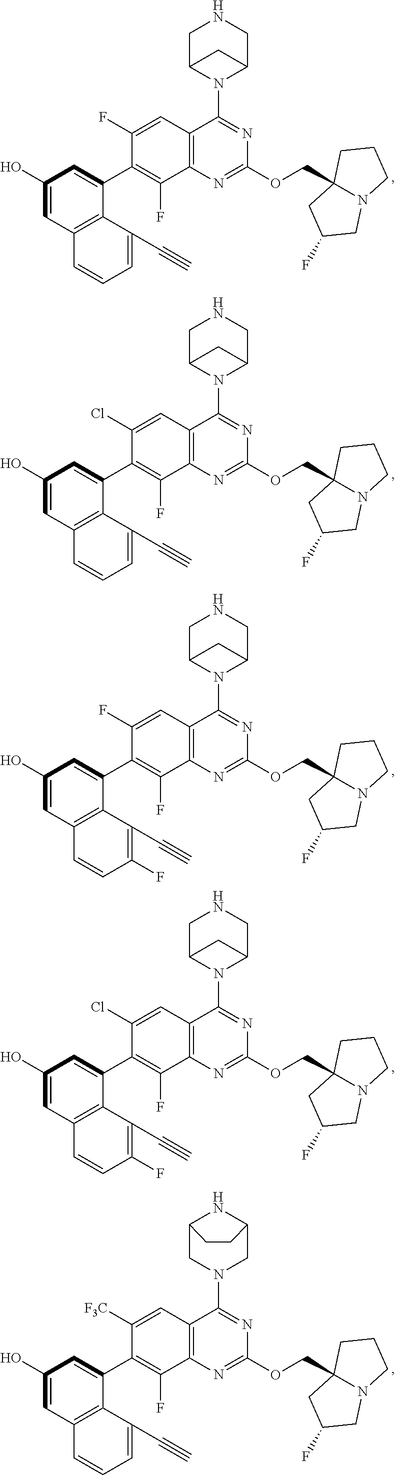

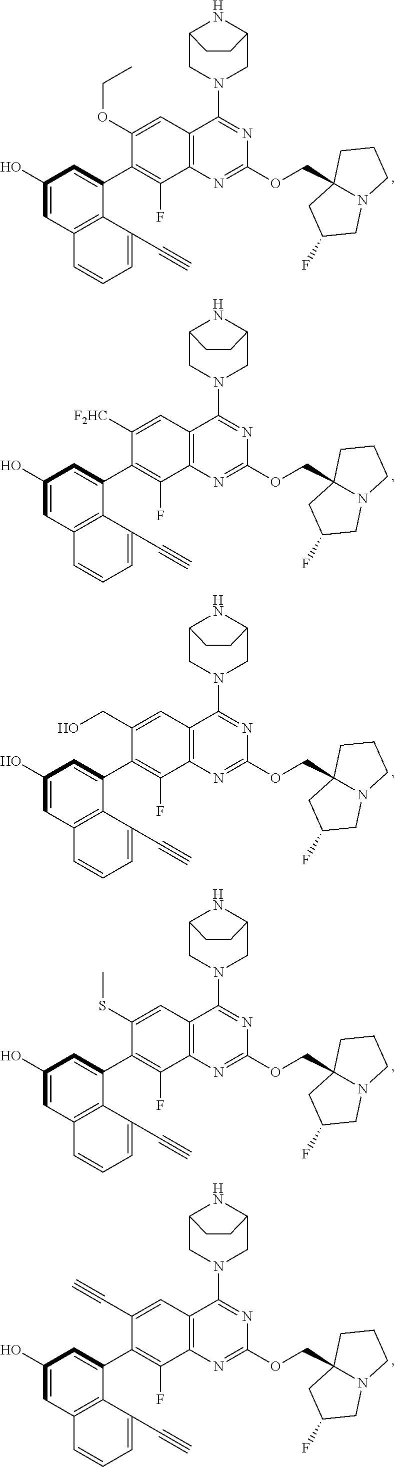

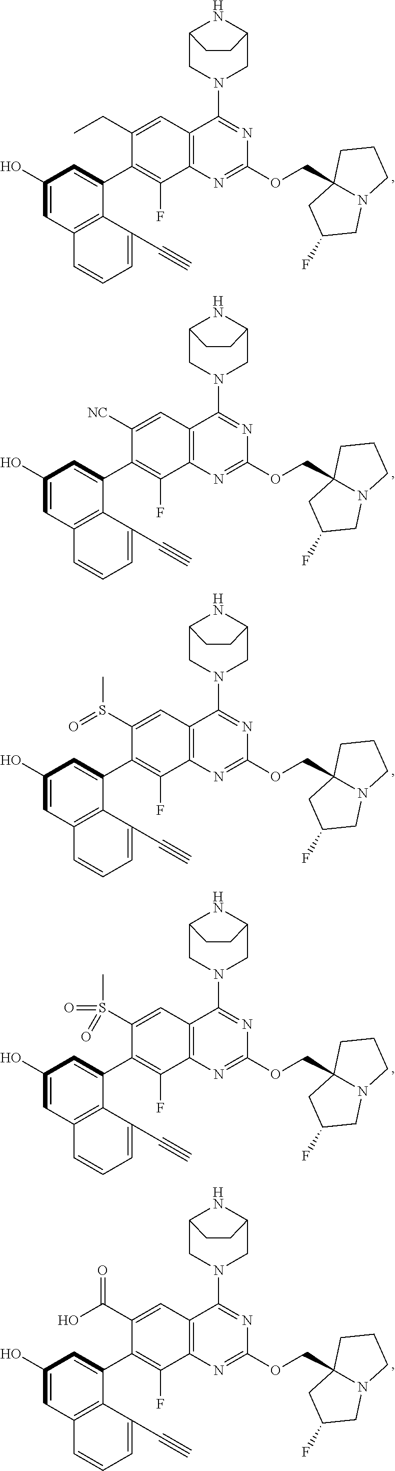

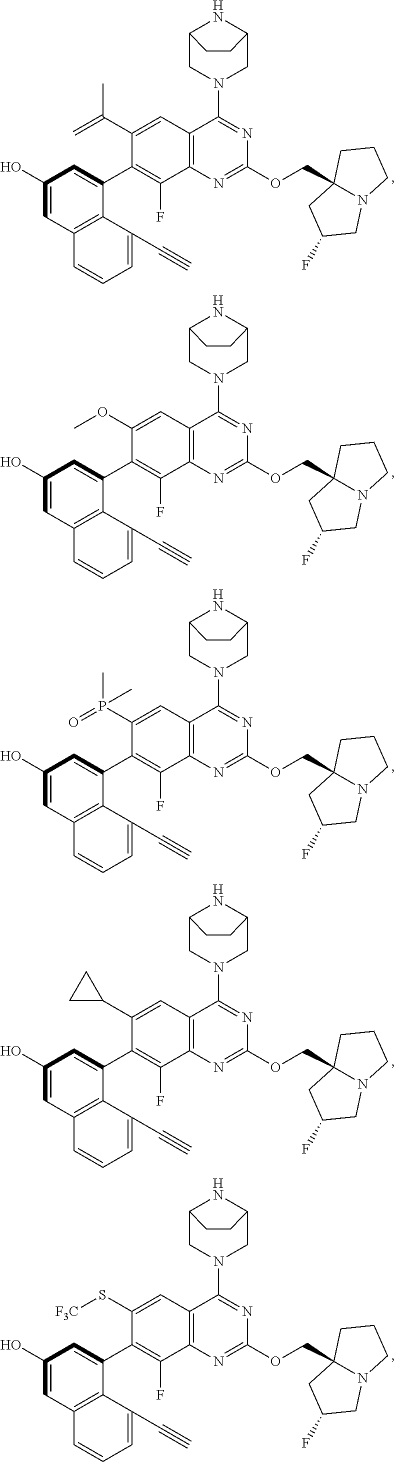

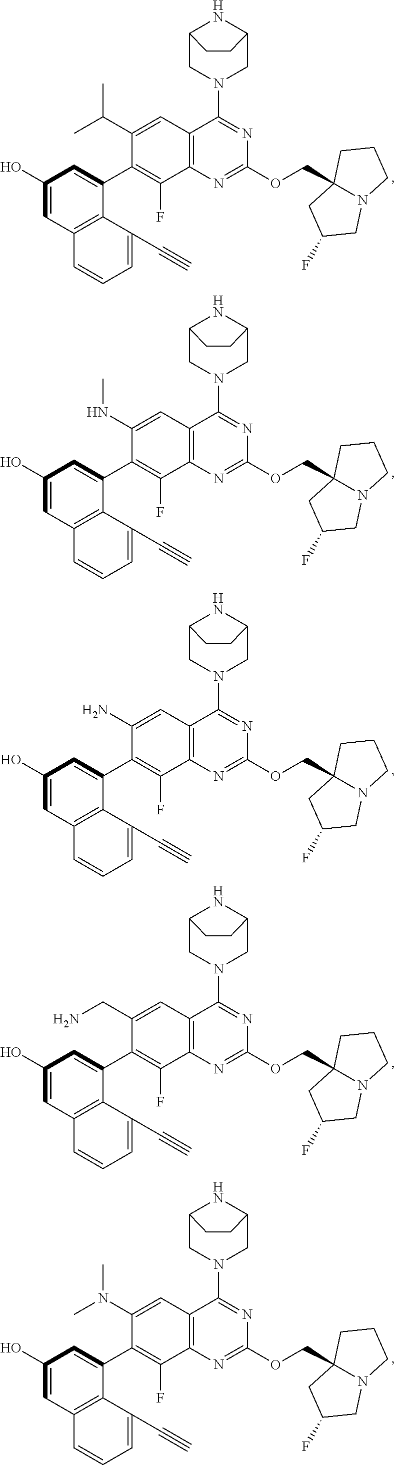

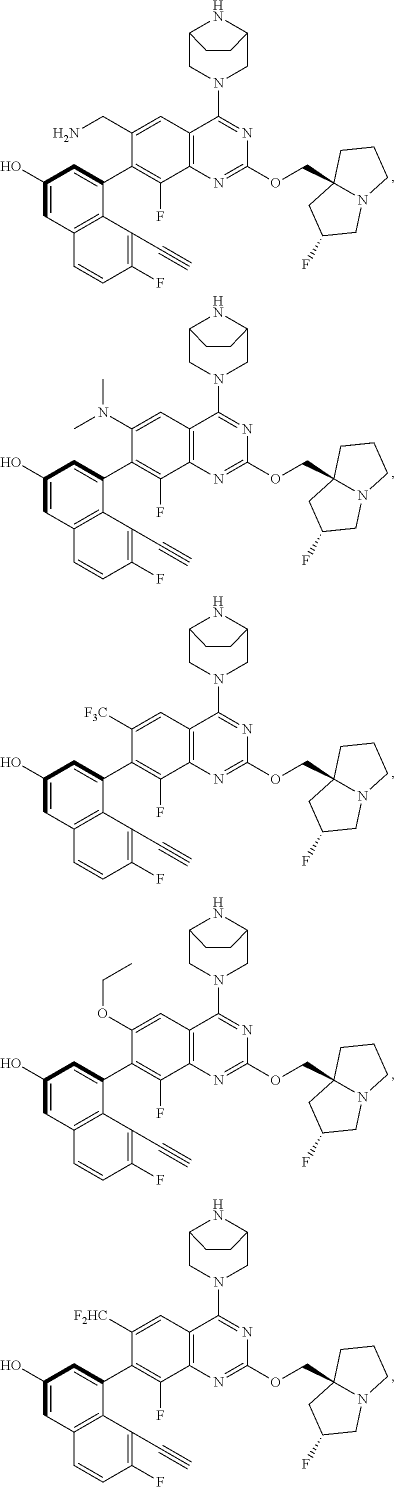

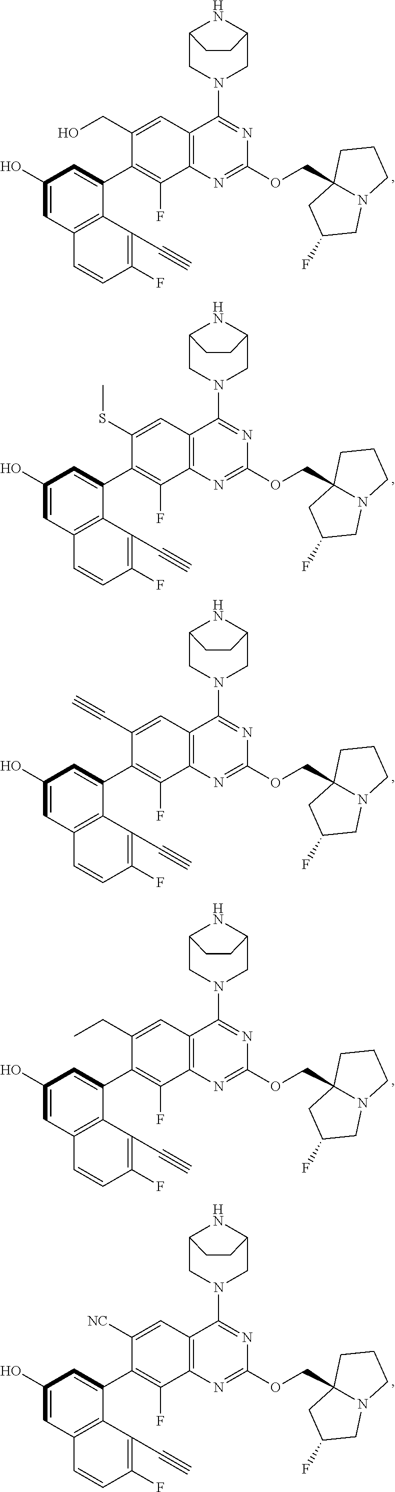

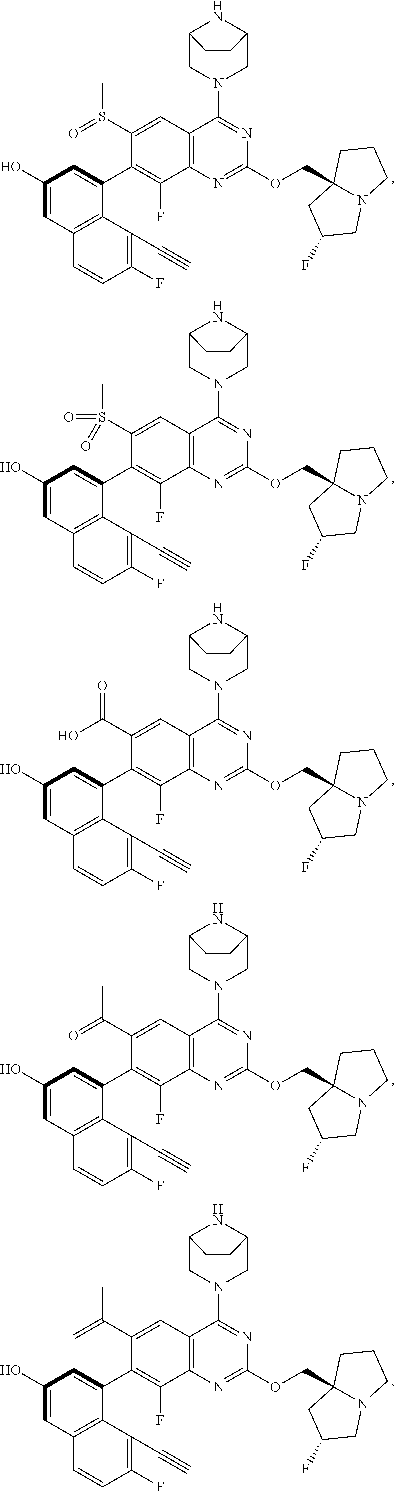

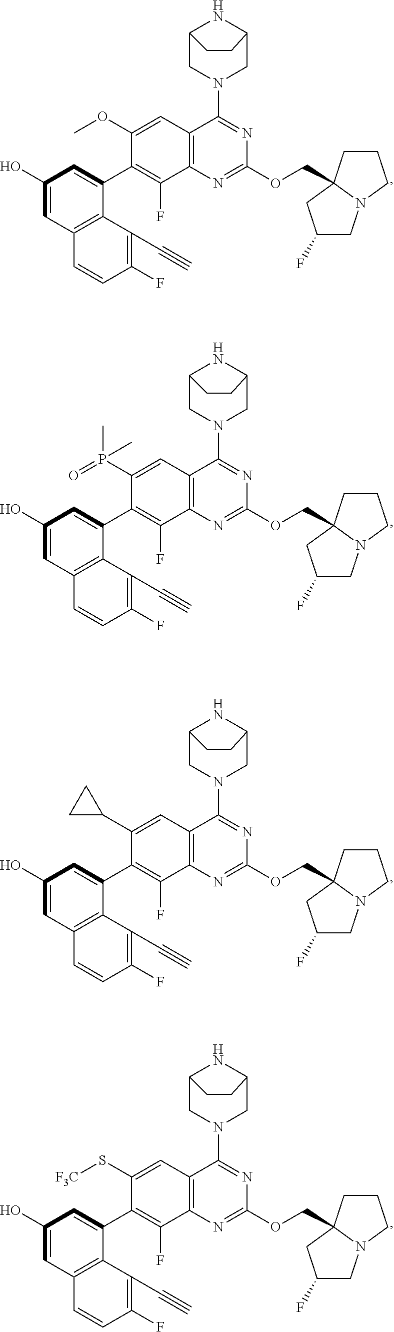

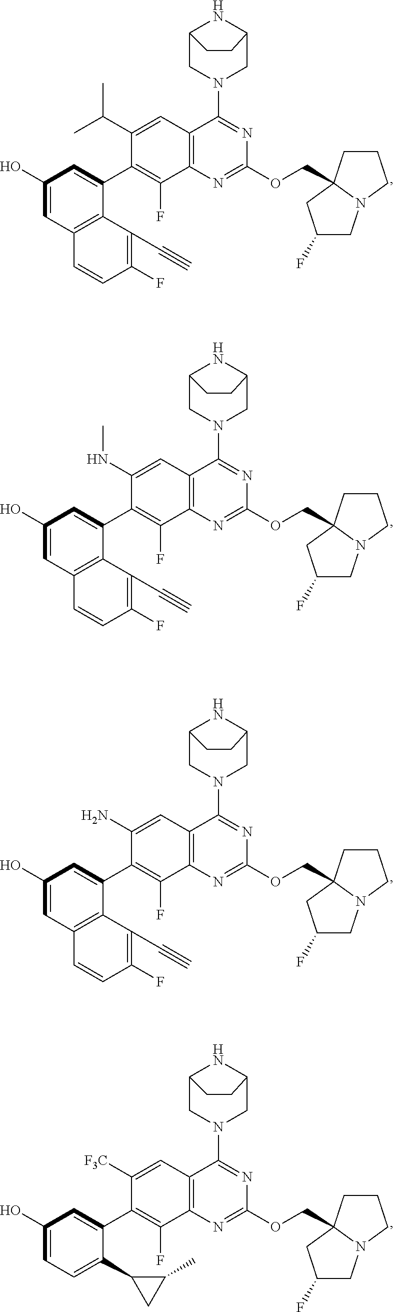

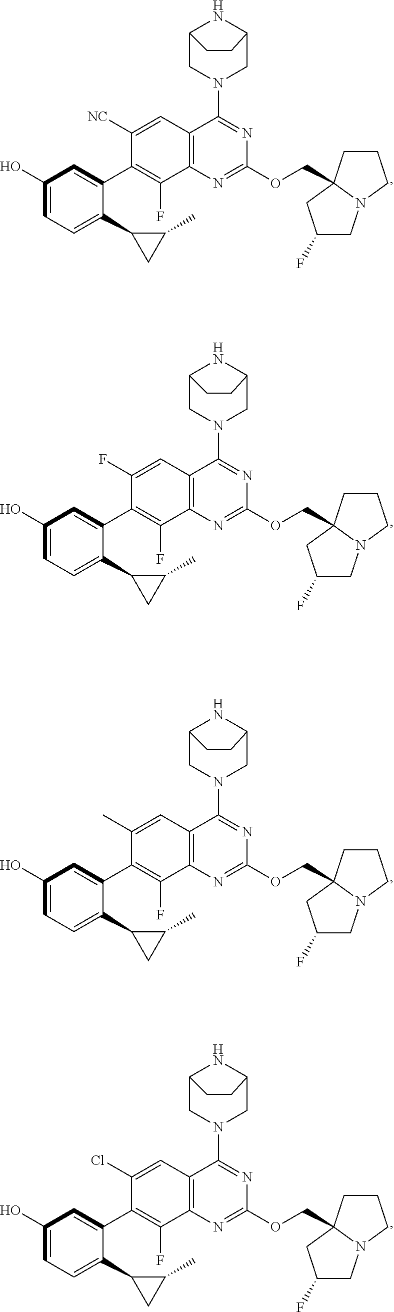

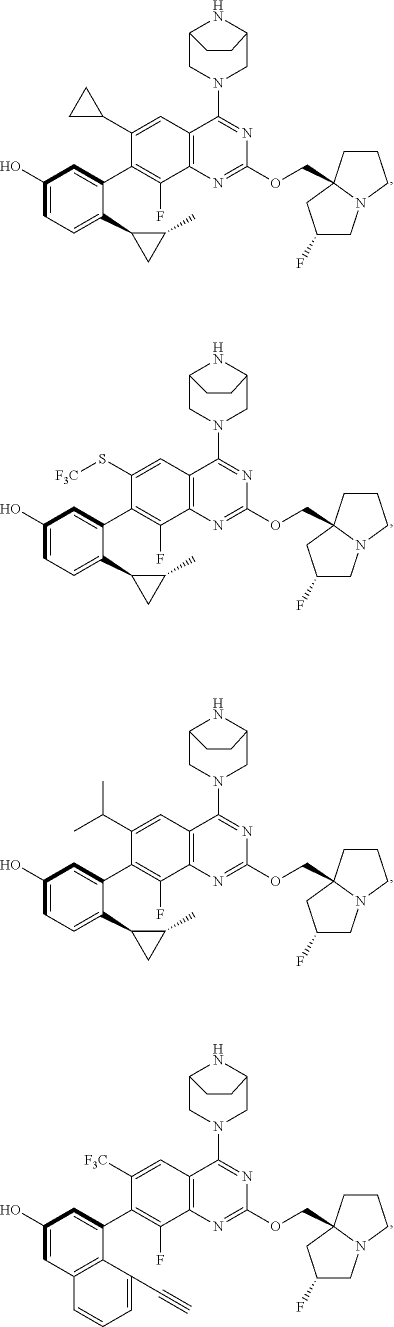

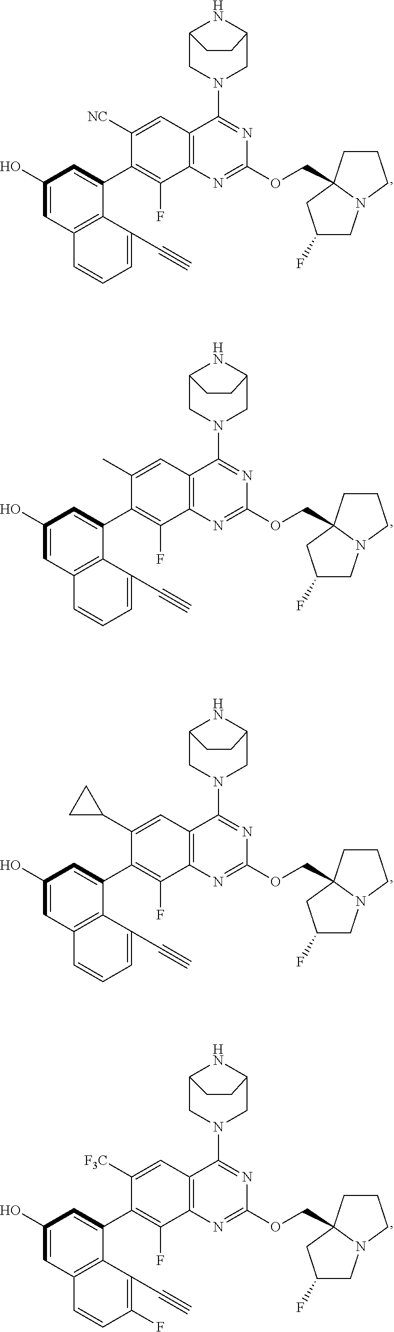

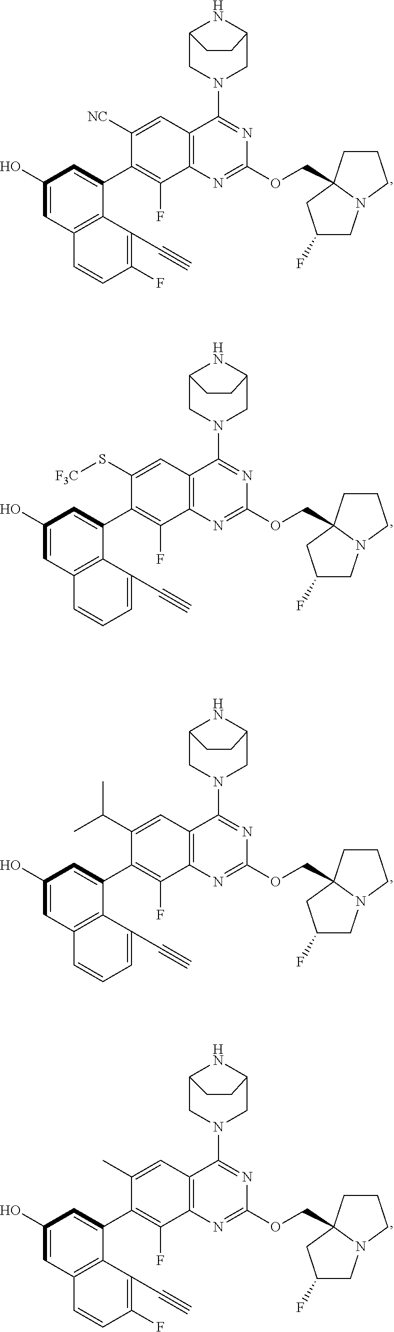

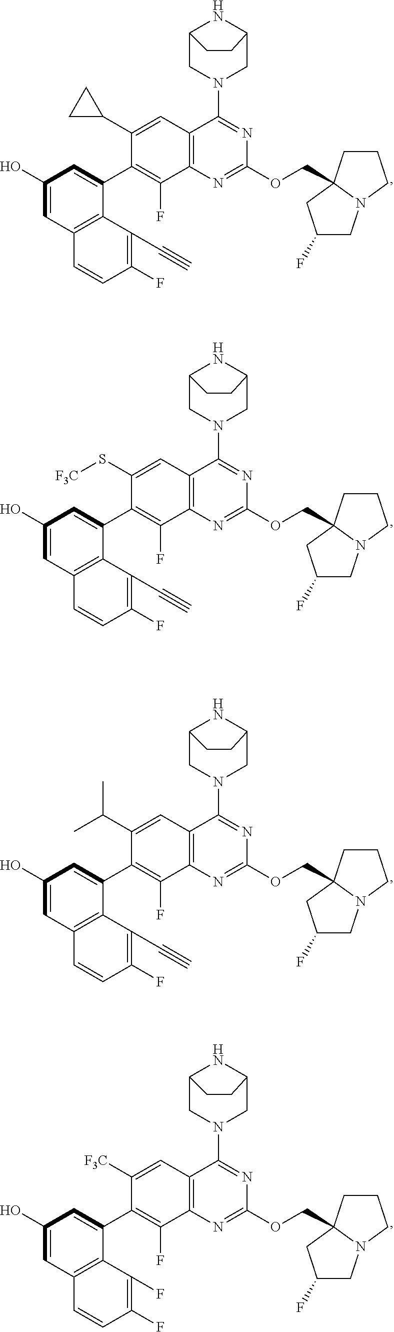

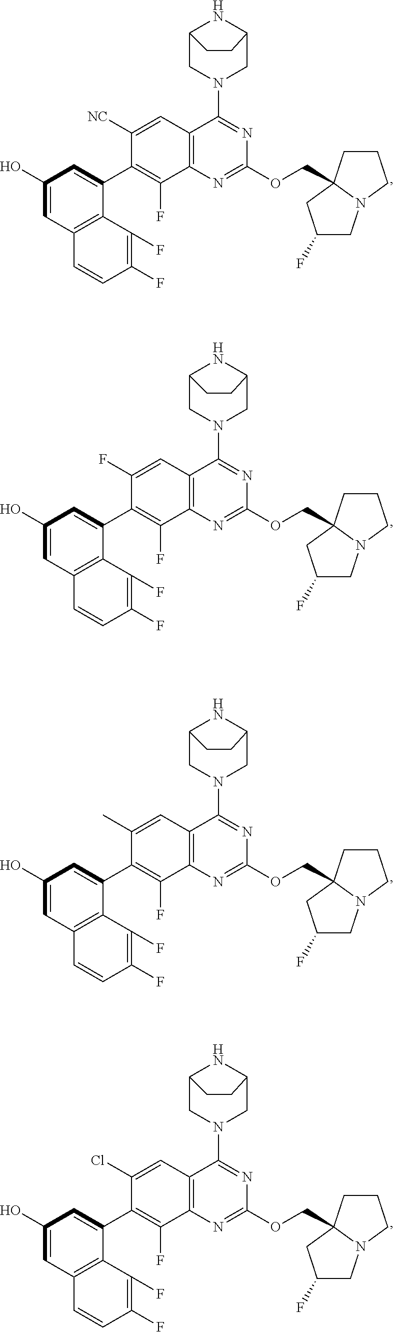

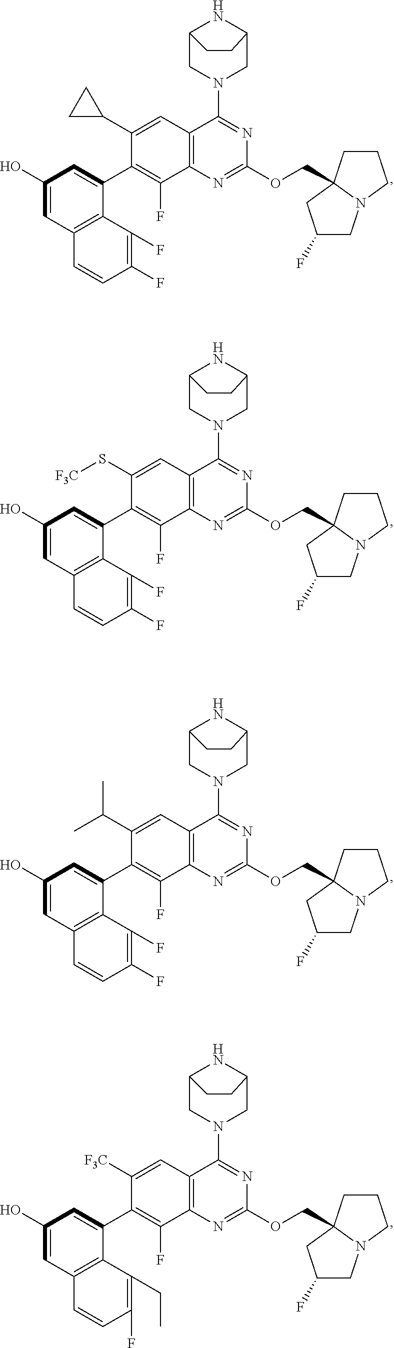

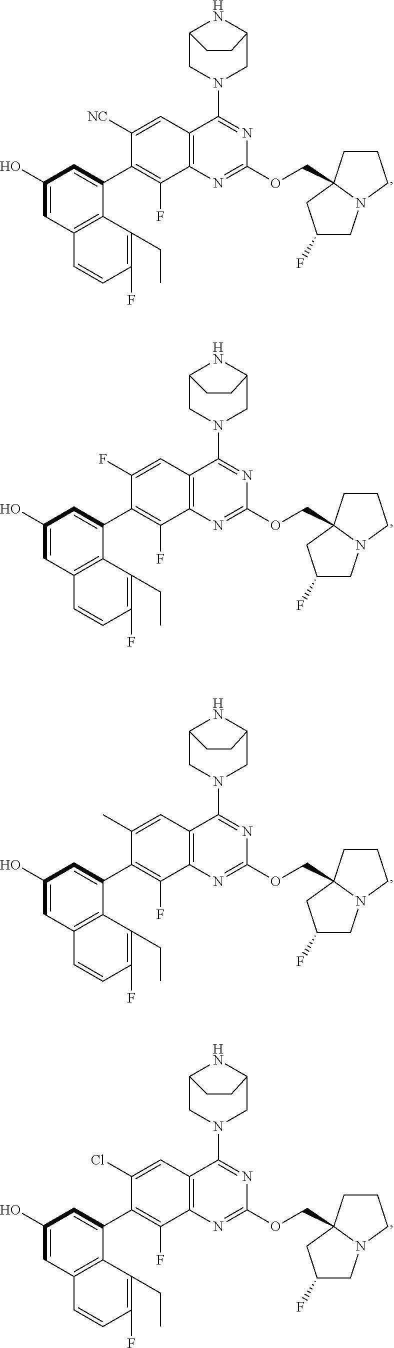

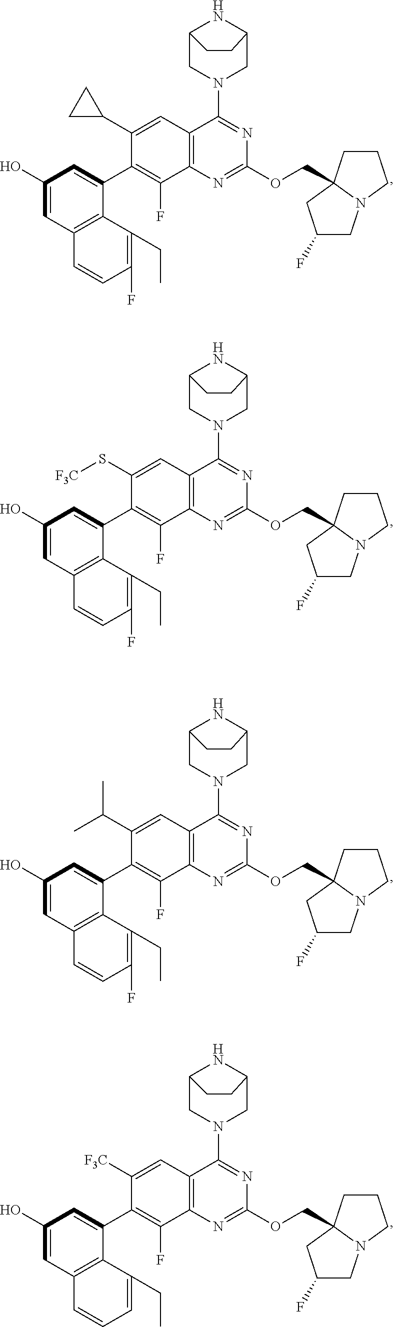

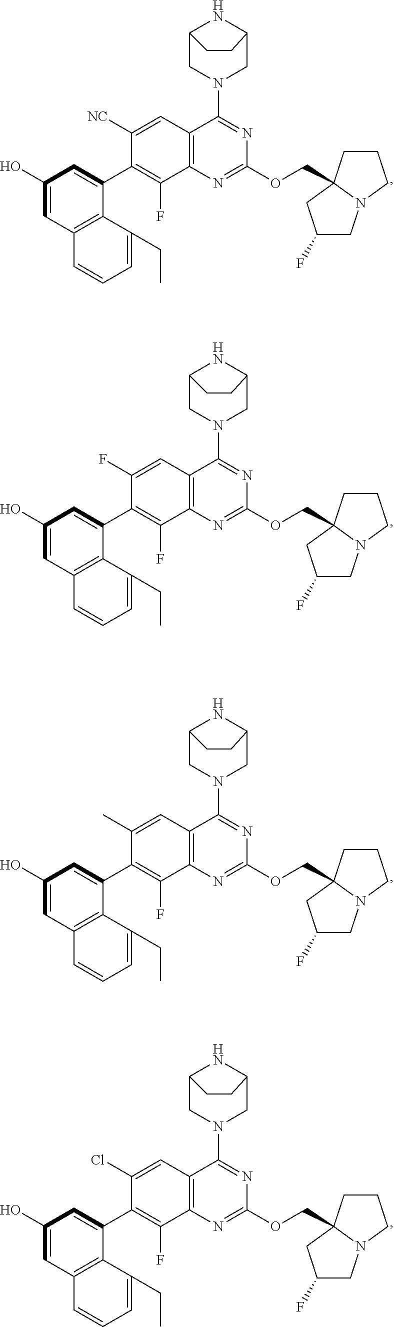

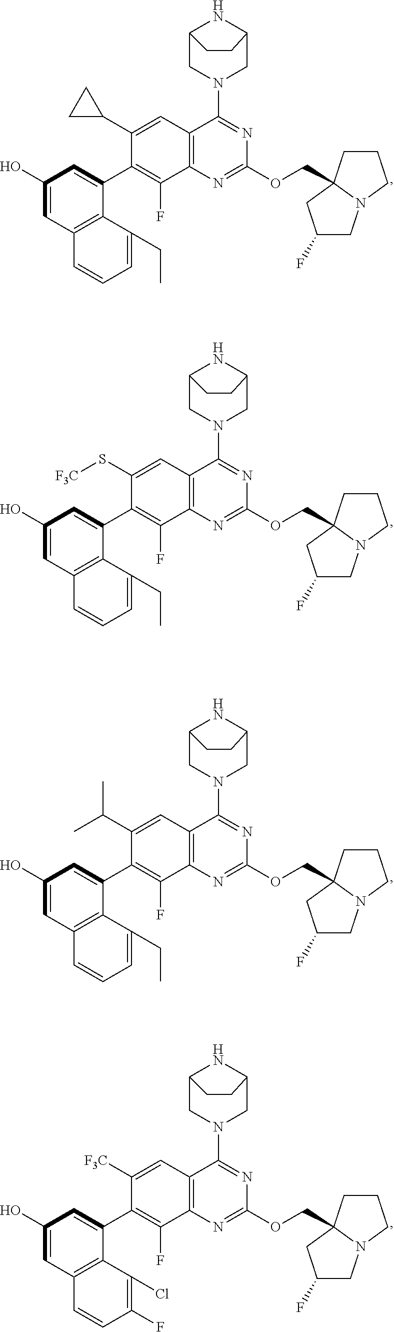

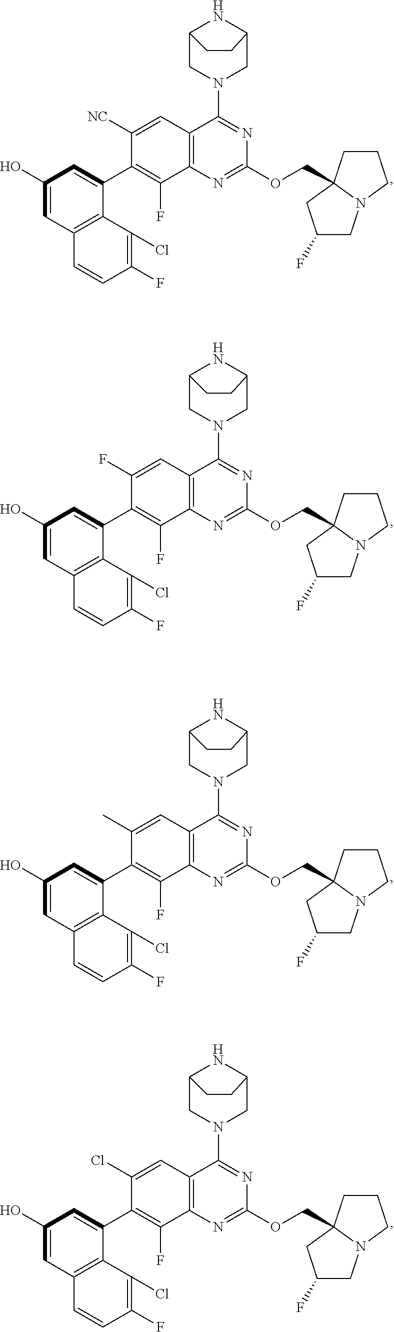

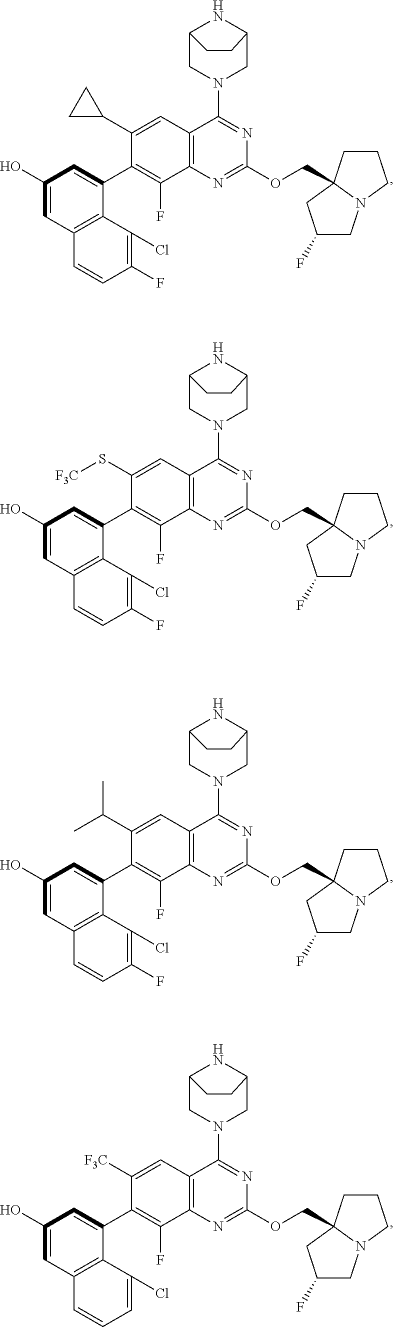

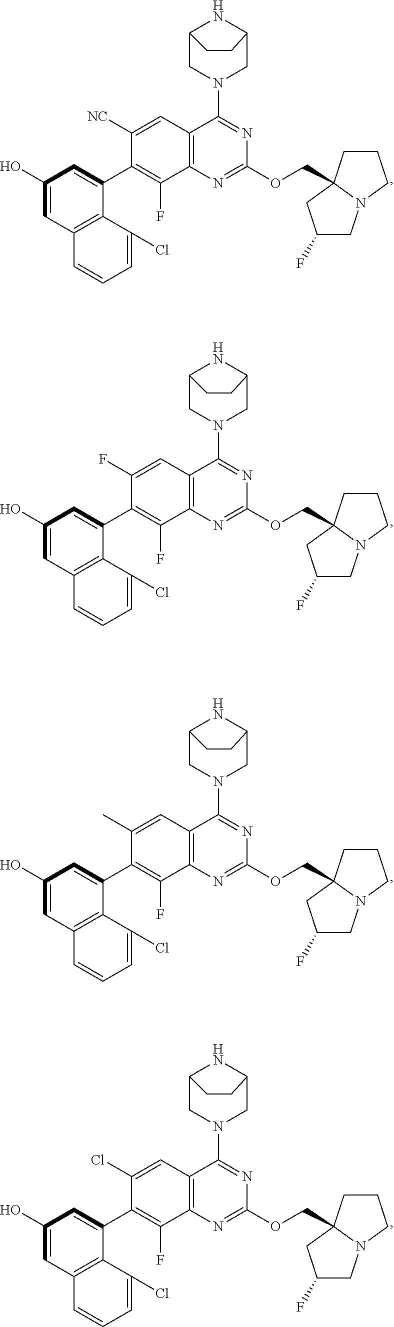

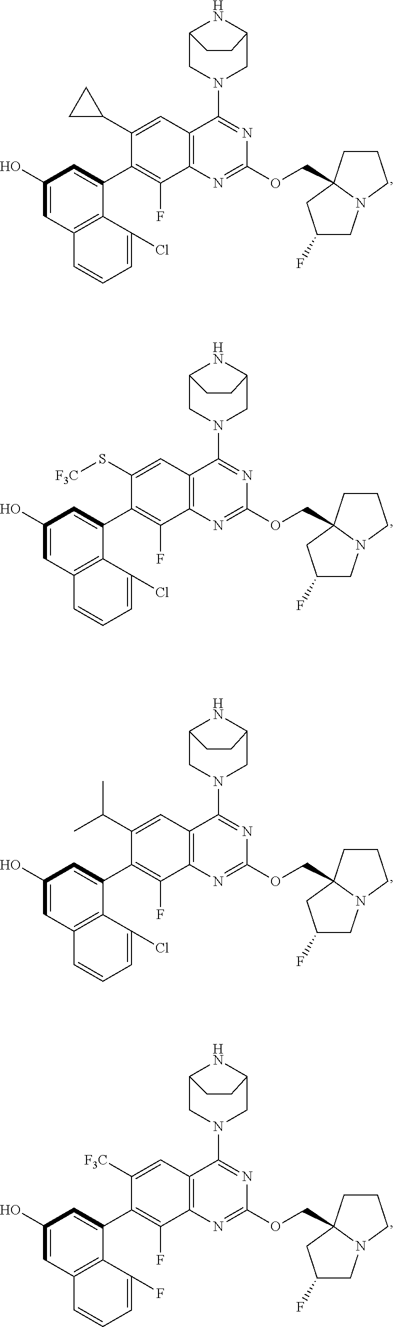

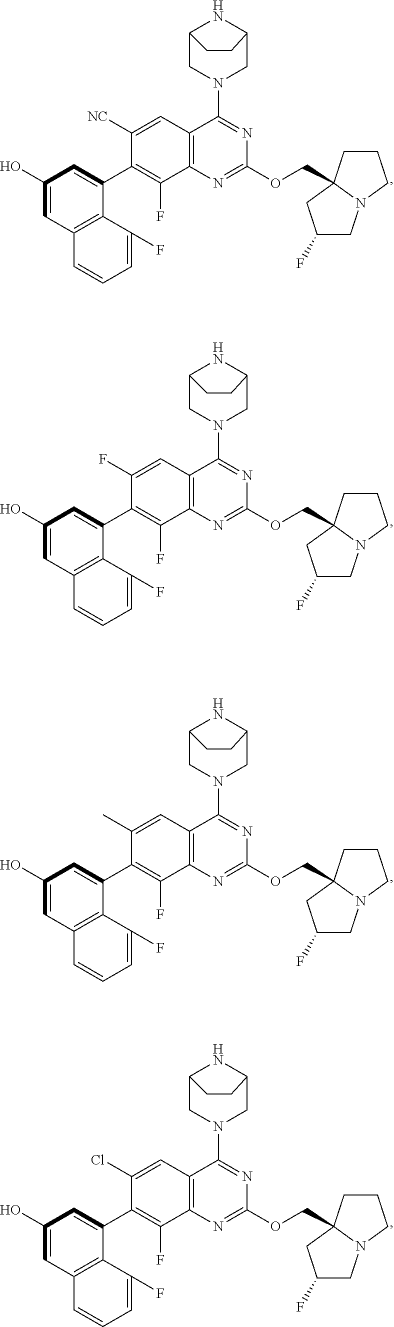

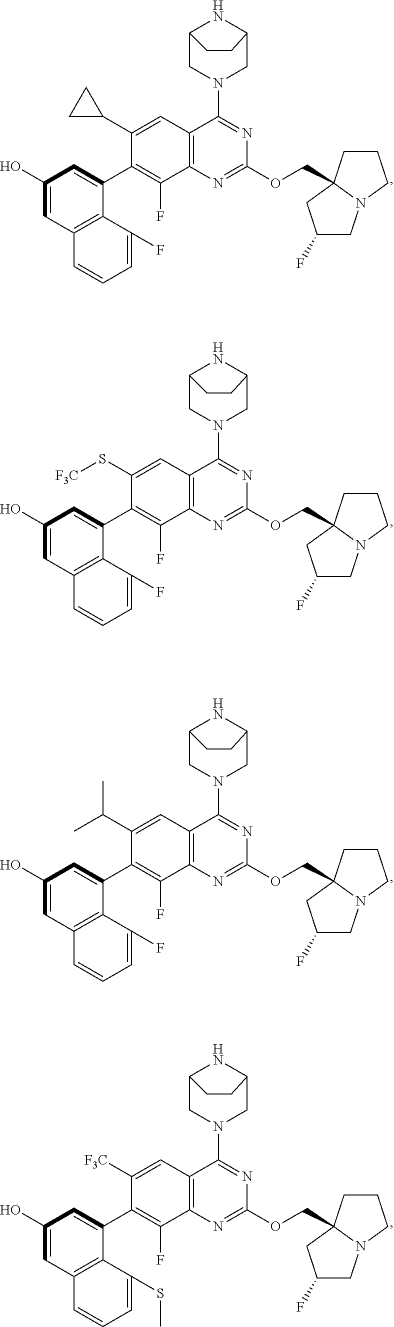

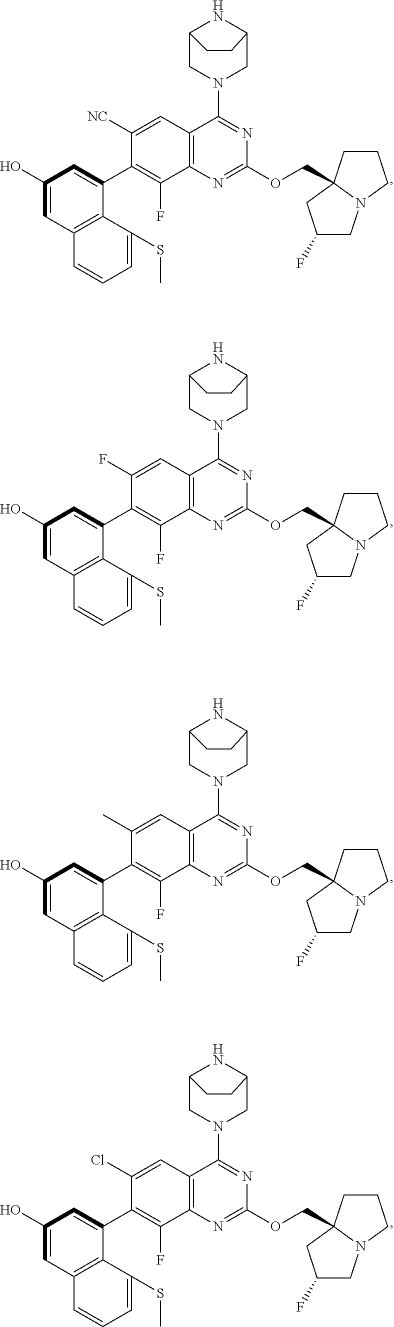

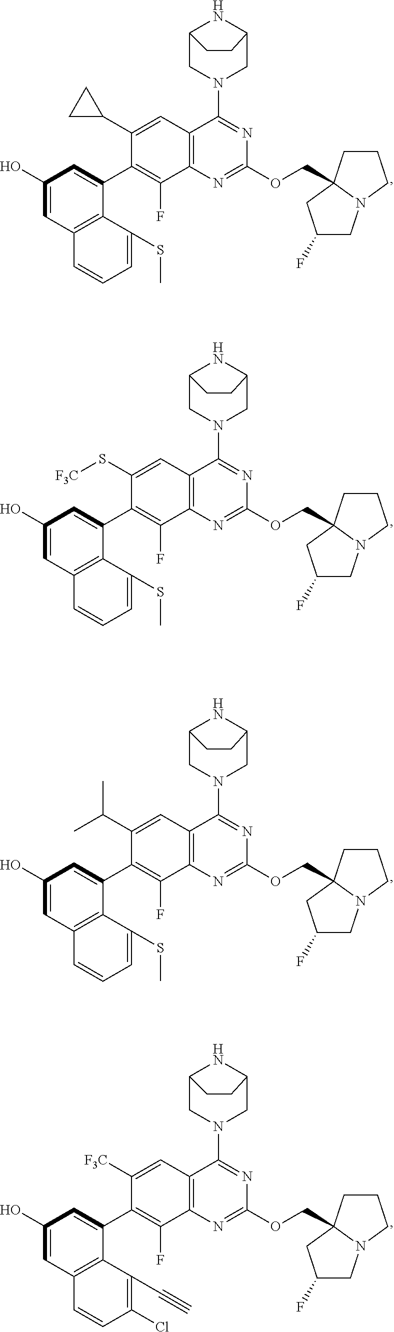

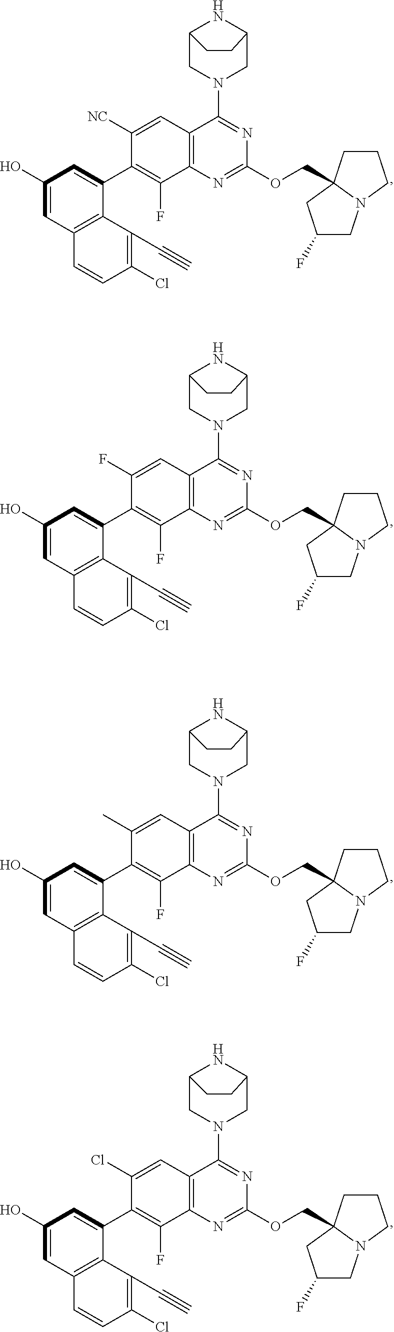

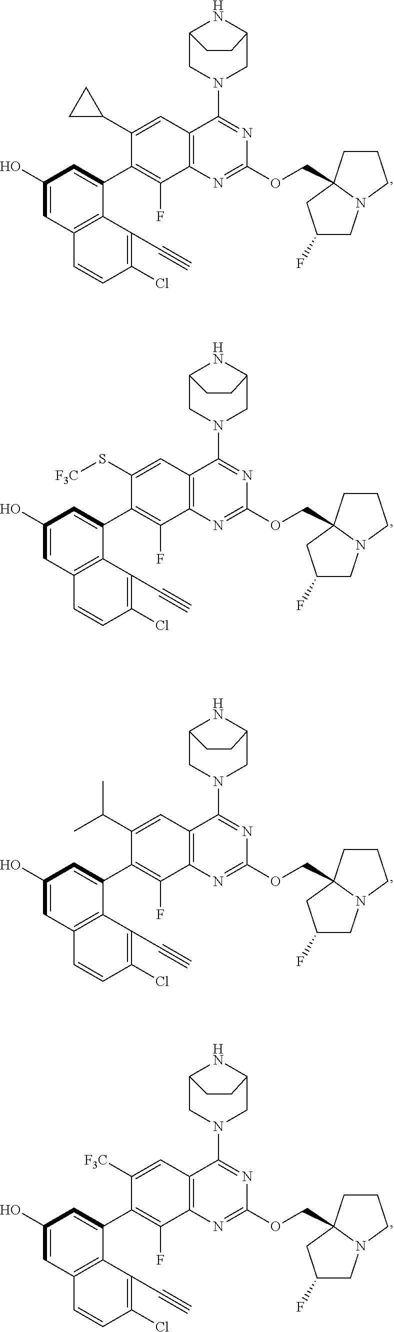

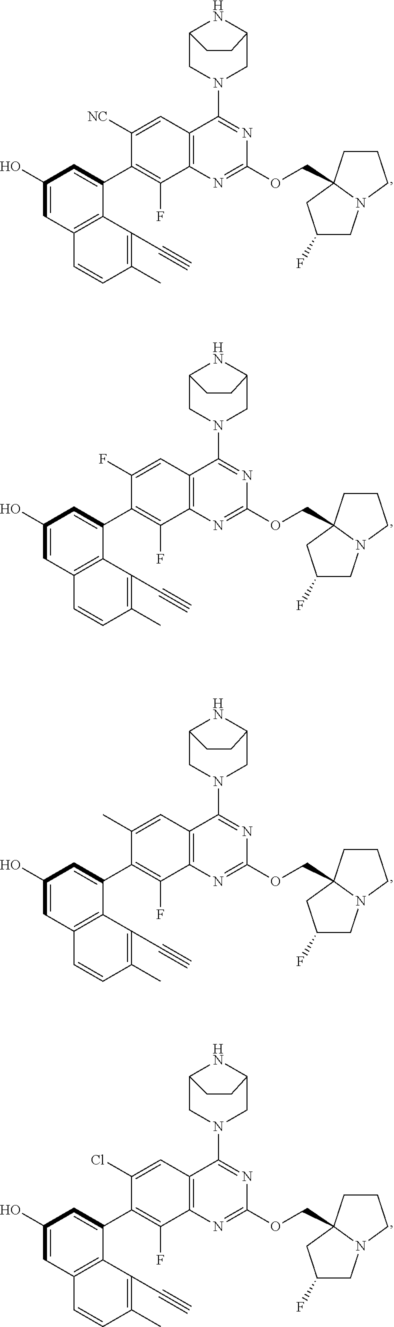

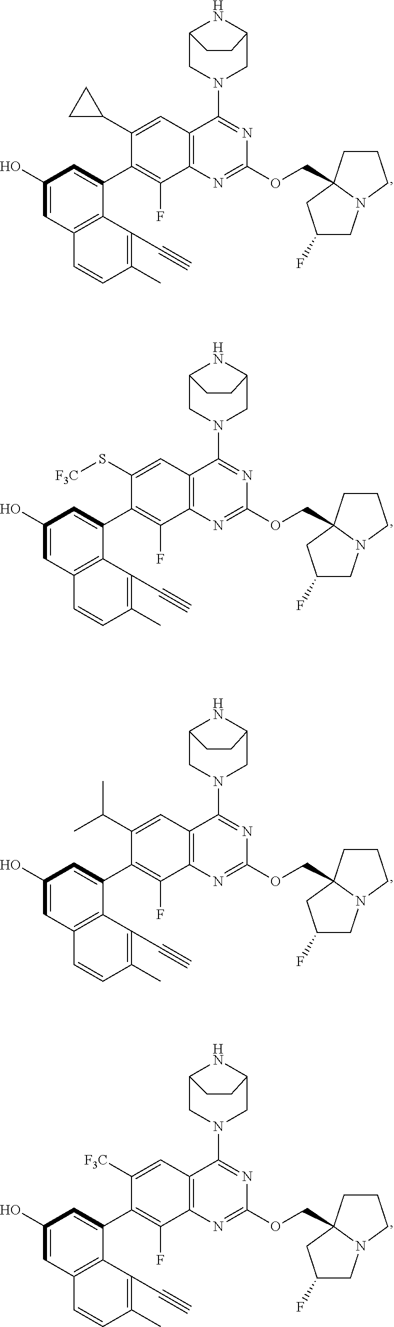

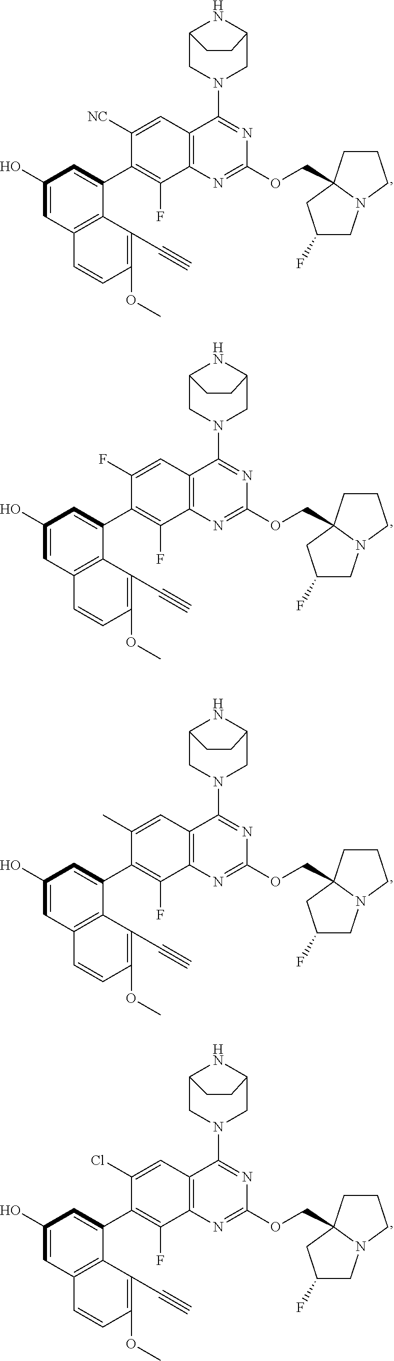

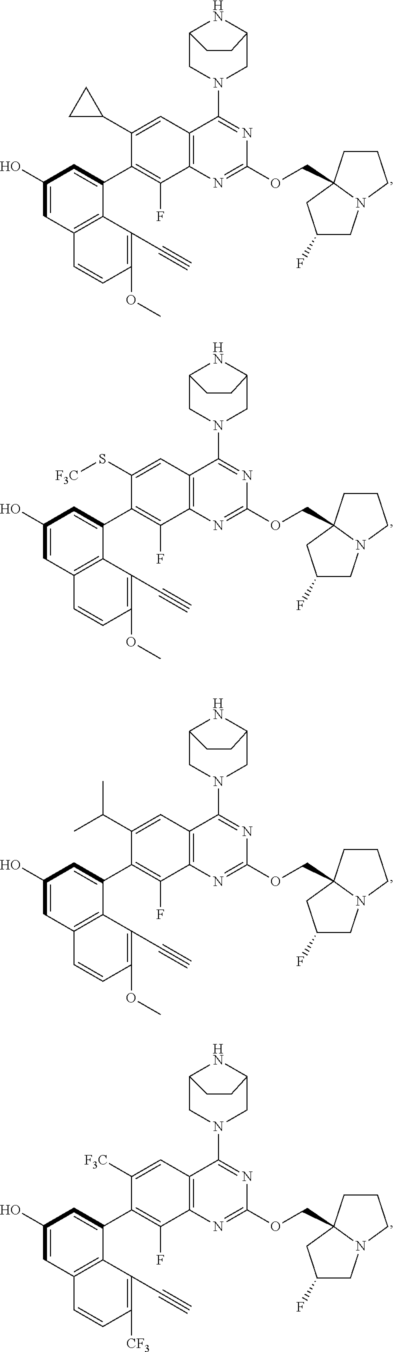

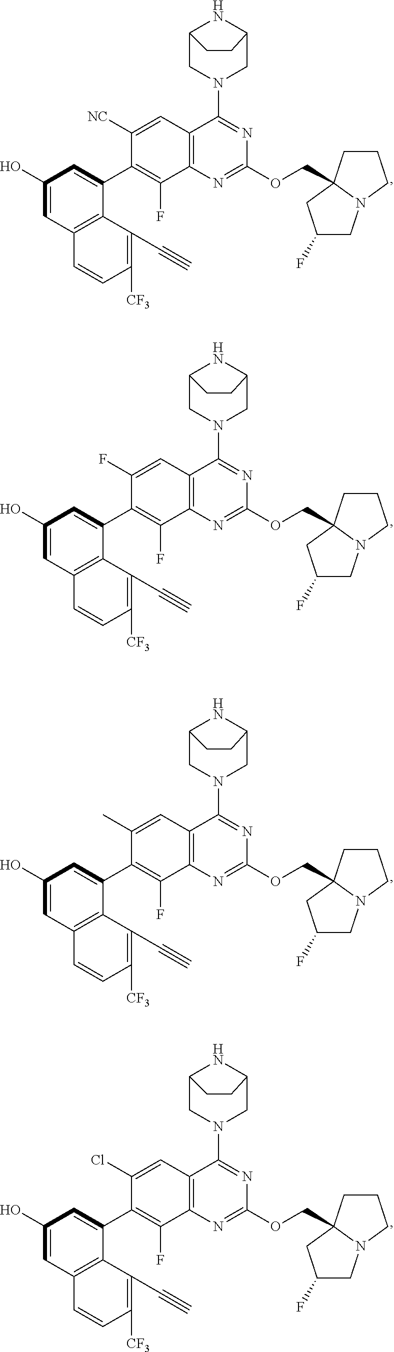

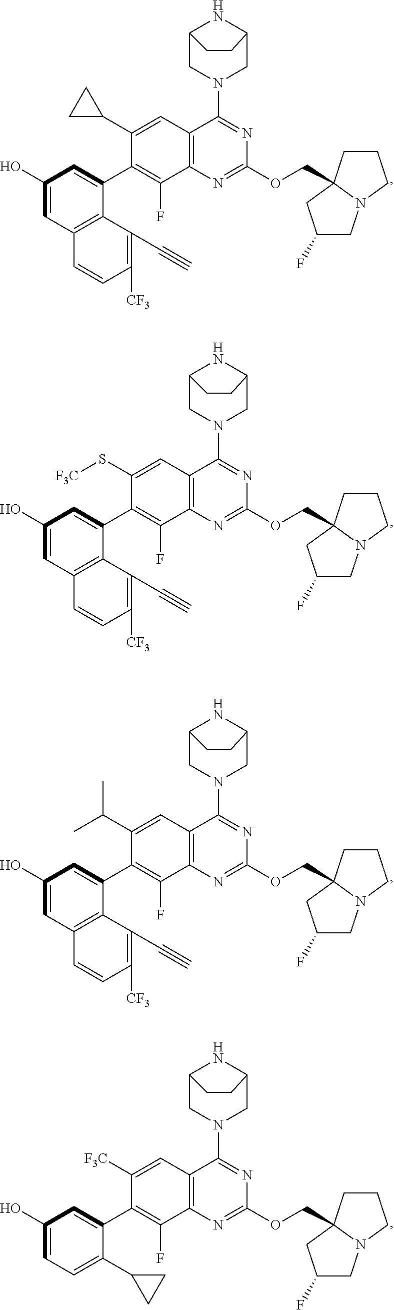

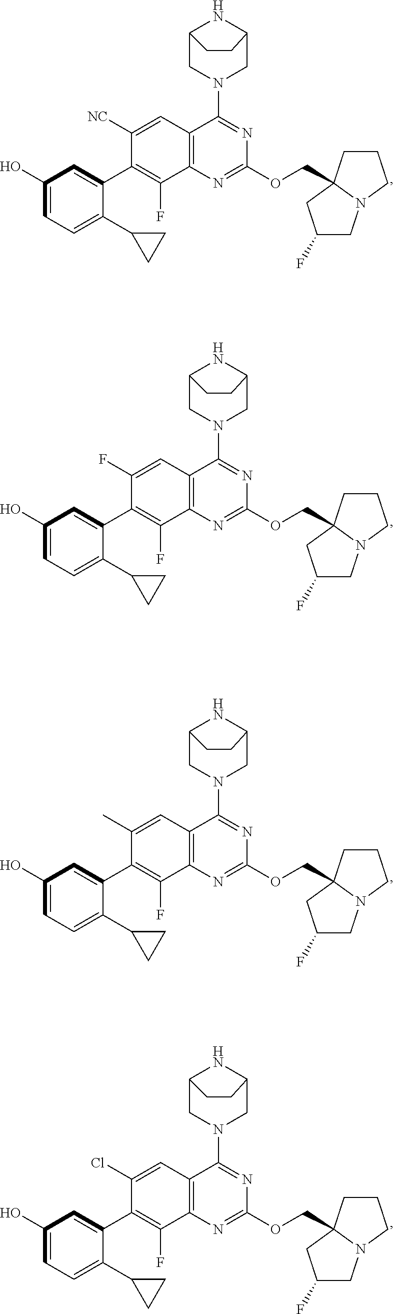

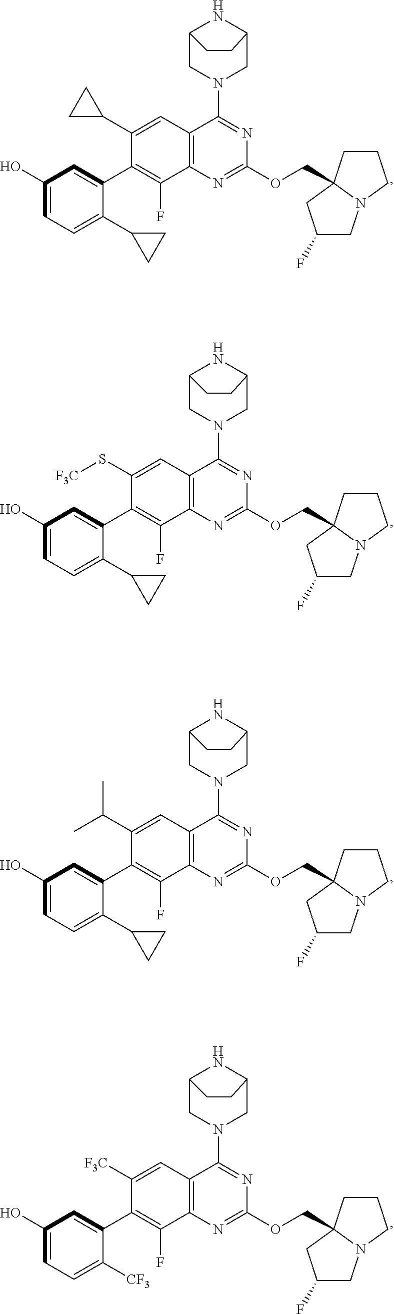

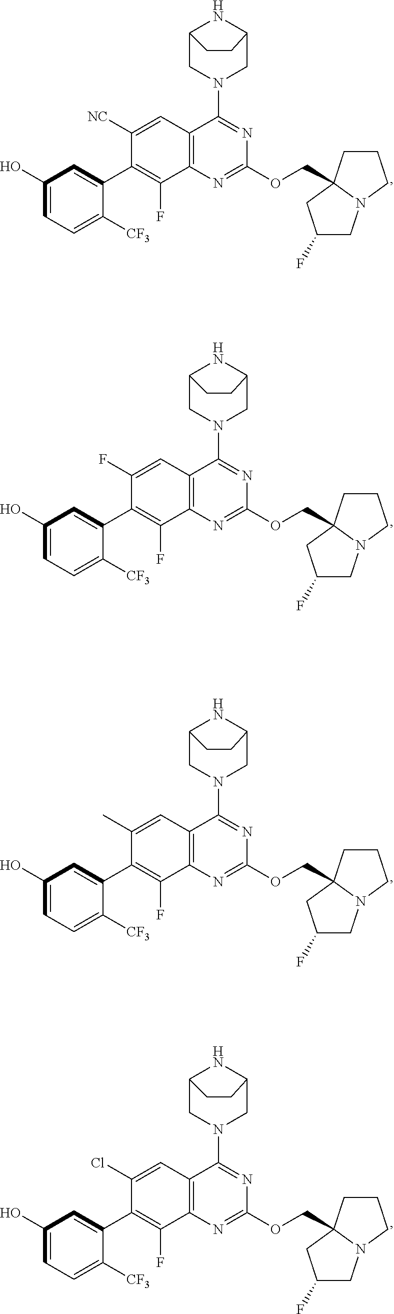

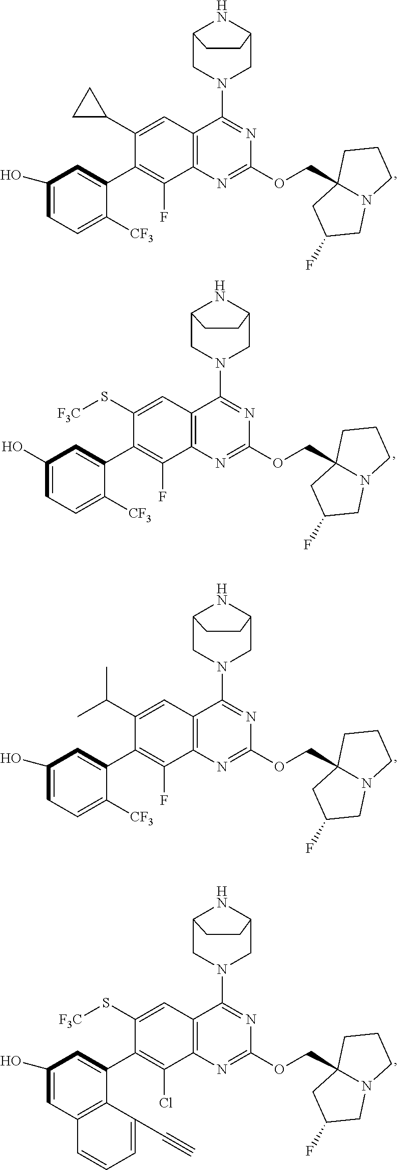

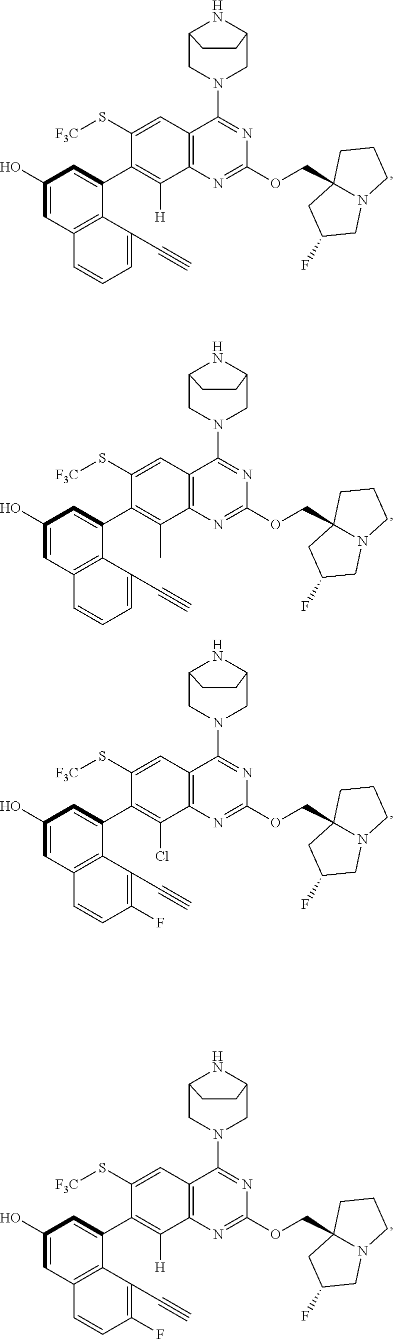

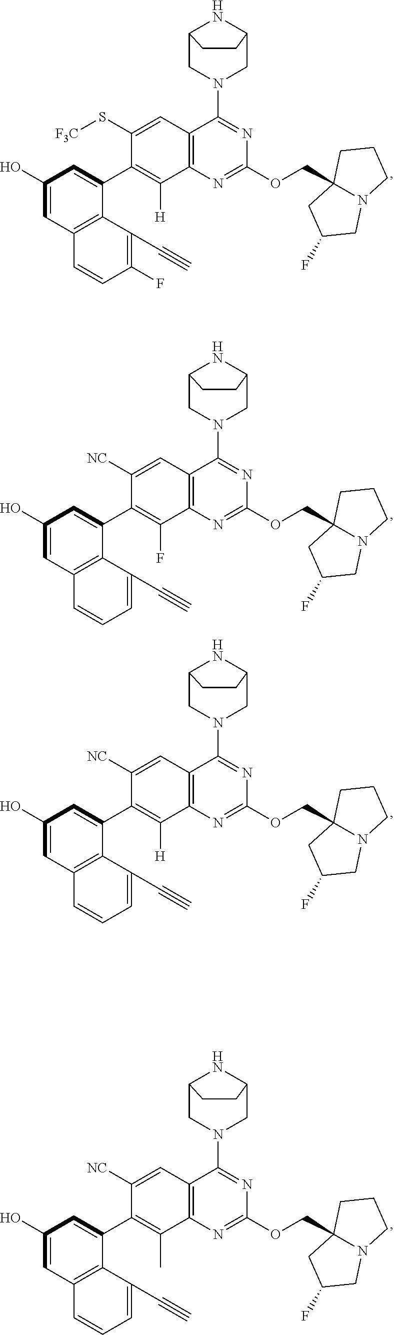

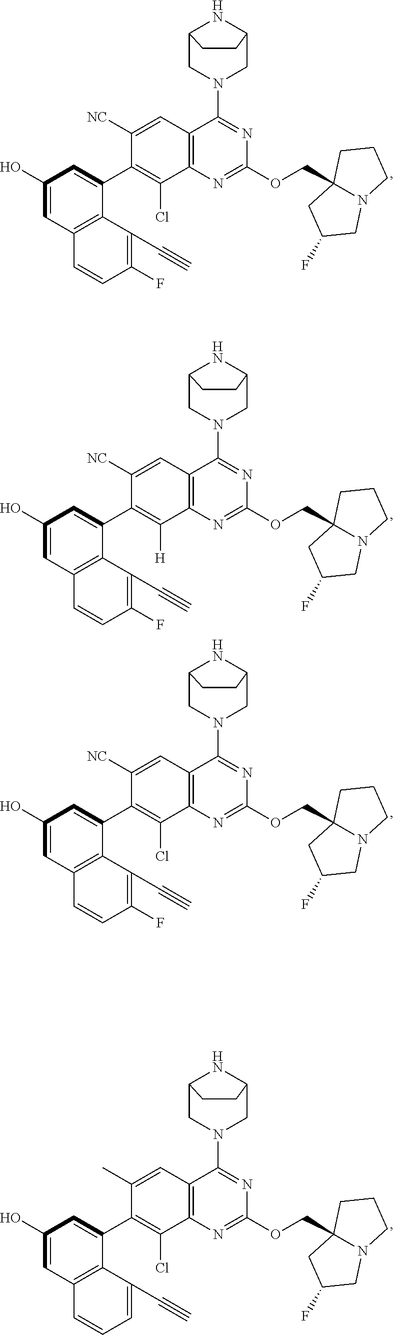

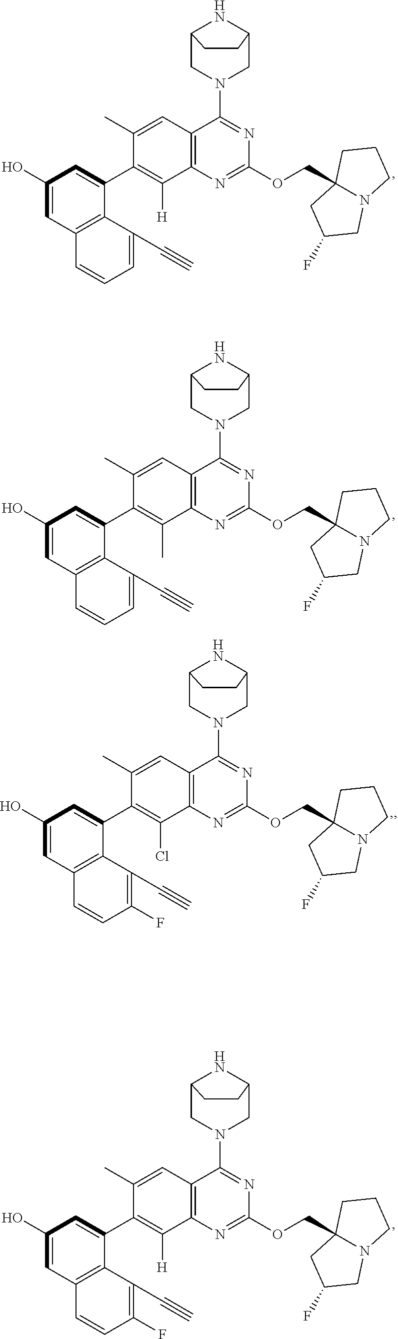

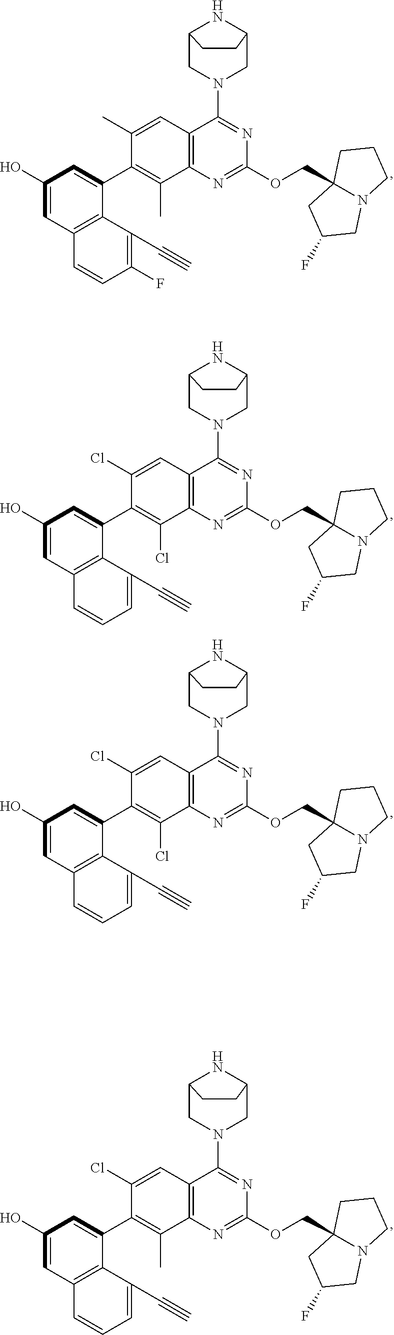

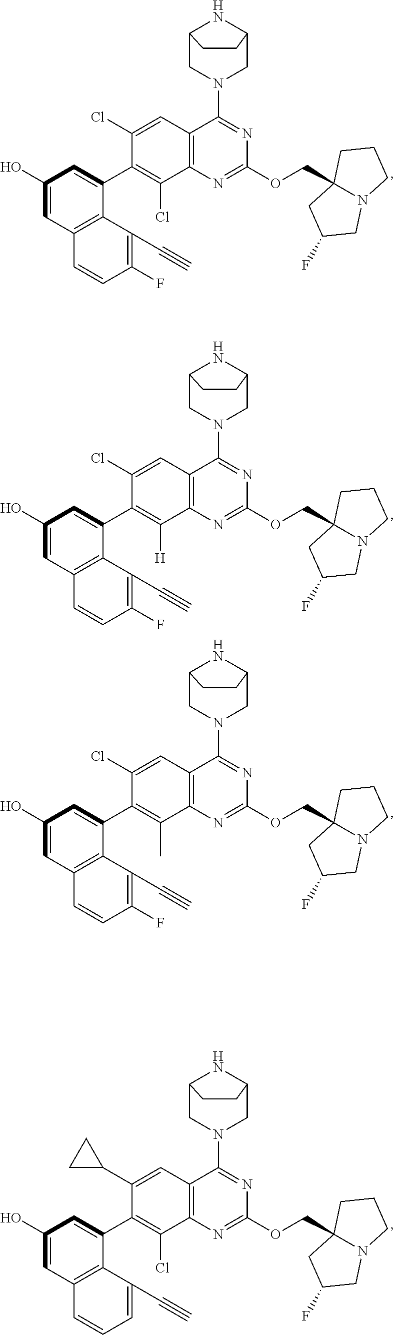

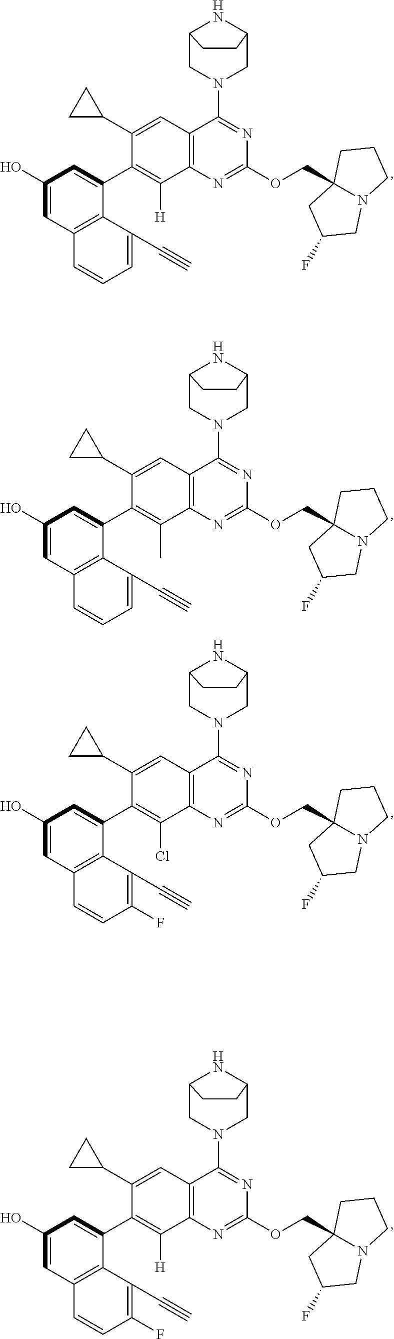

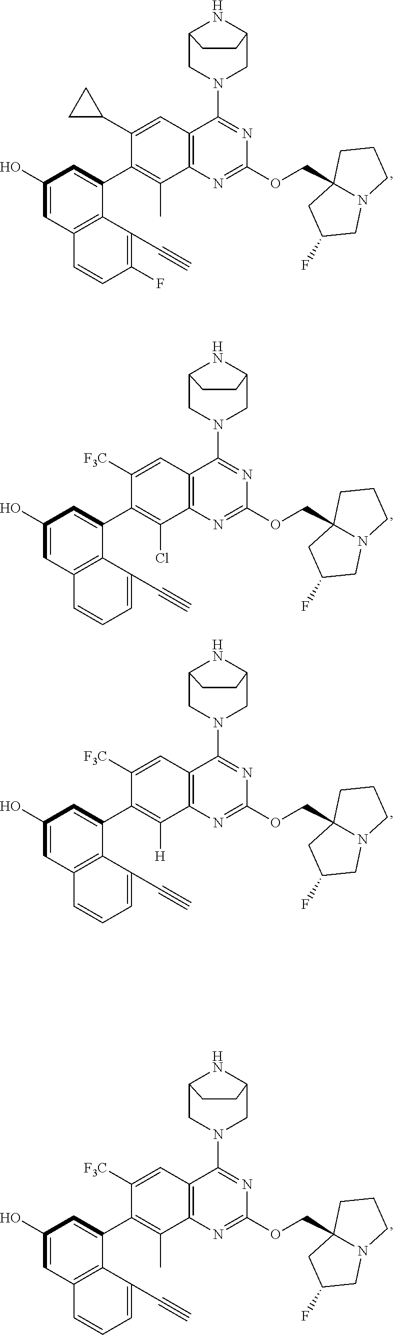

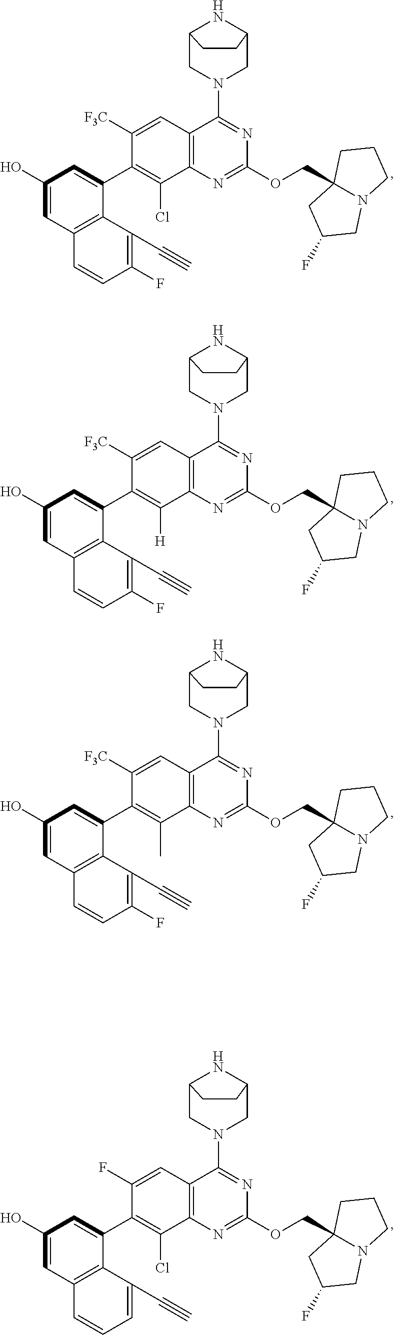

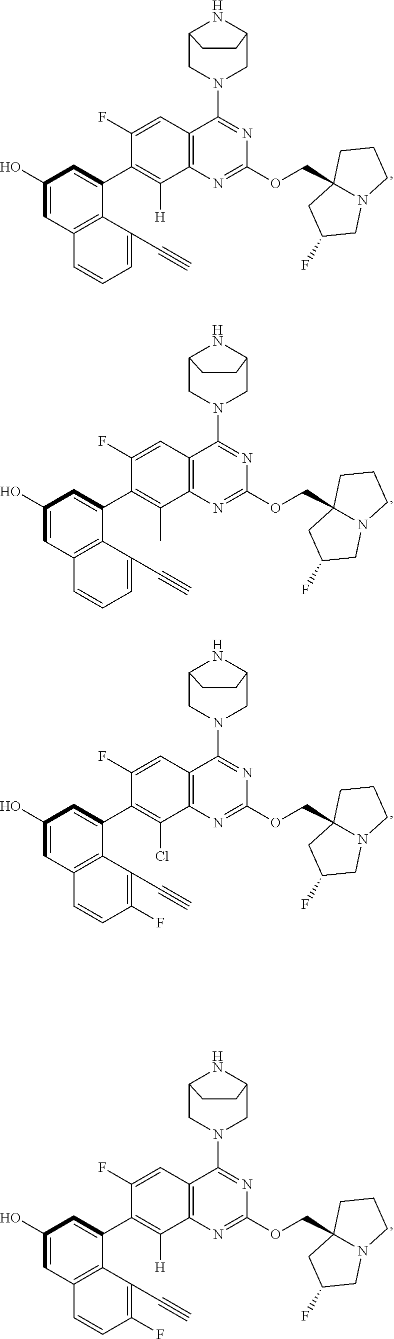

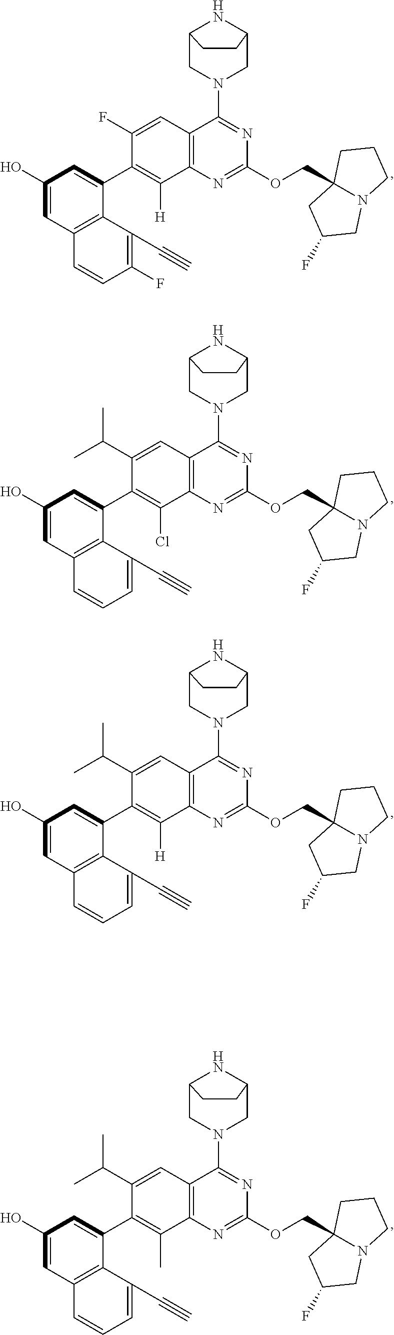

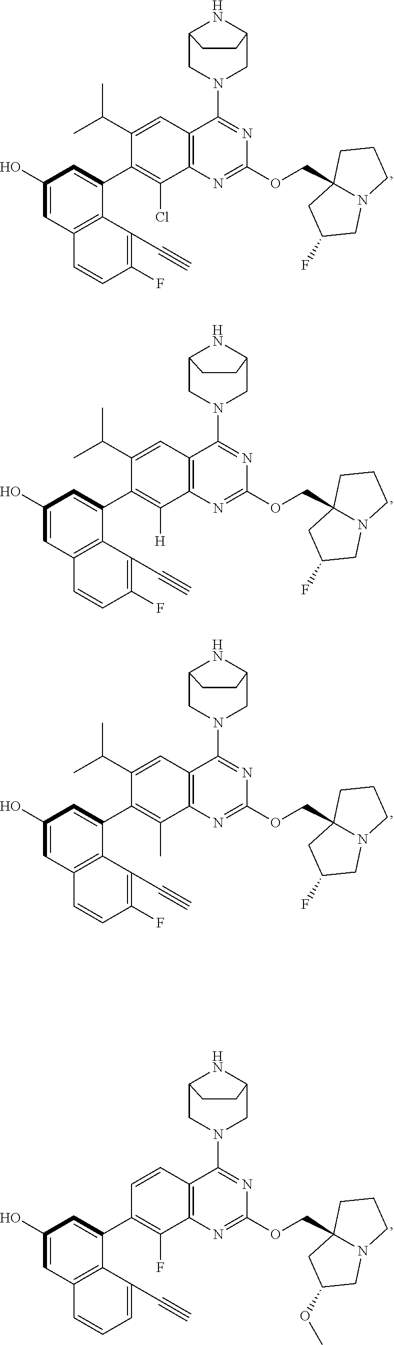

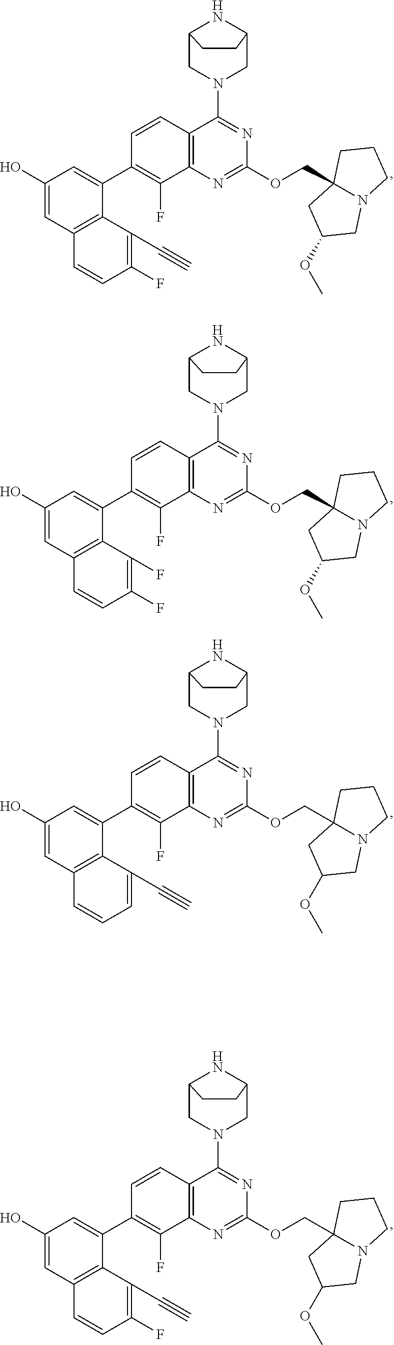 [SMILES: C#Cc1c(F)ccc2cc(O)cc(-c3ccc4c(N5CC6CCC(C5)N6)nc(OC[C@@]56CCCN5C[C@H](OC)C6)nc4c3F)c12 ; CO[C@H]1CN2CCC[C@@]2(COc2nc(N3CC4CCC(C3)N4)c3ccc(-c4cc(O)cc5ccc(F)c(F)c45)c(F)c3n2)C1 ; C#Cc1cccc2cc(O)cc(-c3ccc4c(N5CC6CCC(C5)N6)nc(OCC56CCCN5CC(OC)C6)nc4c3F)c12 ; C#Cc1c(F)ccc2cc(O)cc(-c3ccc4c(N5CC6CCC(C5)N6)nc(OCC56CCCN5CC(OC)C6)nc4c3F)c12]

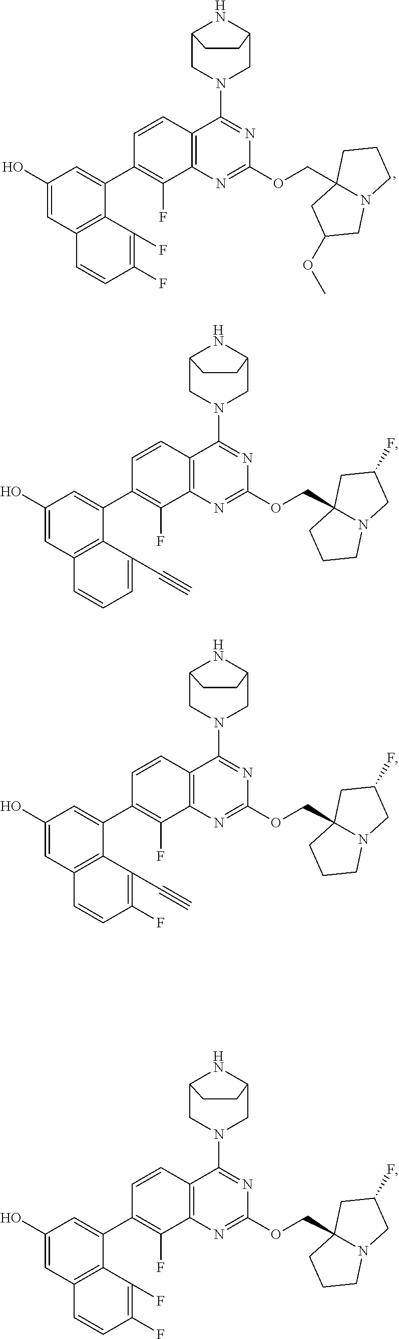 [SMILES: COC1CN2CCCC2(COc2nc(N3CC4CCC(C3)N4)c3ccc(-c4cc(O)cc5ccc(F)c(F)c45)c(F)c3n2)C1 ; C#Cc1cccc2cc(O)cc(-c3ccc4c(N5CC6CCC(C5)N6)nc(OC[C@]56CCCN5C[C@@H](F)C6)nc4c3F)c12 ; C#Cc1c(F)ccc2cc(O)cc(-c3ccc4c(N5CC6CCC(C5)N6)nc(OC[C@]56CCCN5C[C@@H](F)C6)nc4c3F)c12 ; Oc1cc(-c2ccc3c(N4CC5CCC(C4)N5)nc(OC[C@]45CCCN4C[C@@H](F)C5)nc3c2F)c2c(F)c(F)ccc2c1]

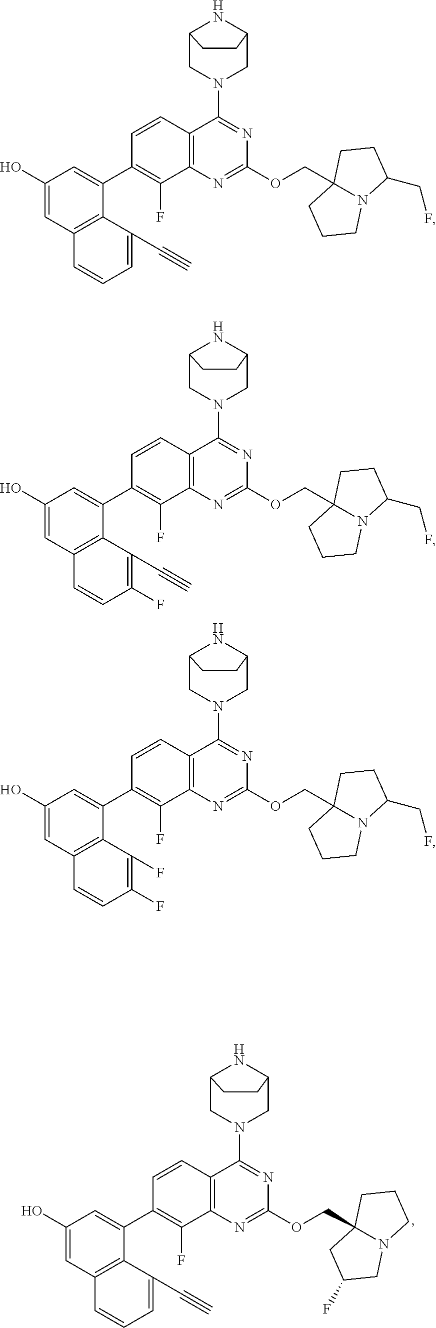 [SMILES: C#Cc1cccc2cc(O)cc(-c3ccc4c(N5CC6CCC(C5)N6)nc(OCC56CCCN5C(CF)CC6)nc4c3F)c12 ; C#Cc1c(F)ccc2cc(O)cc(-c3ccc4c(N5CC6CCC(C5)N6)nc(OCC56CCCN5C(CF)CC6)nc4c3F)c12 ; Oc1cc(-c2ccc3c(N4CC5CCC(C4)N5)nc(OCC45CCCN4C(CF)CC5)nc3c2F)c2c(F)c(F)ccc2c1 ; C#Cc1cccc2cc(O)cc(-c3ccc4c(N5CC6CCC(C5)N6)nc(OC[C@@]56CCCN5C[C@H](F)C6)nc4c3F)c12]

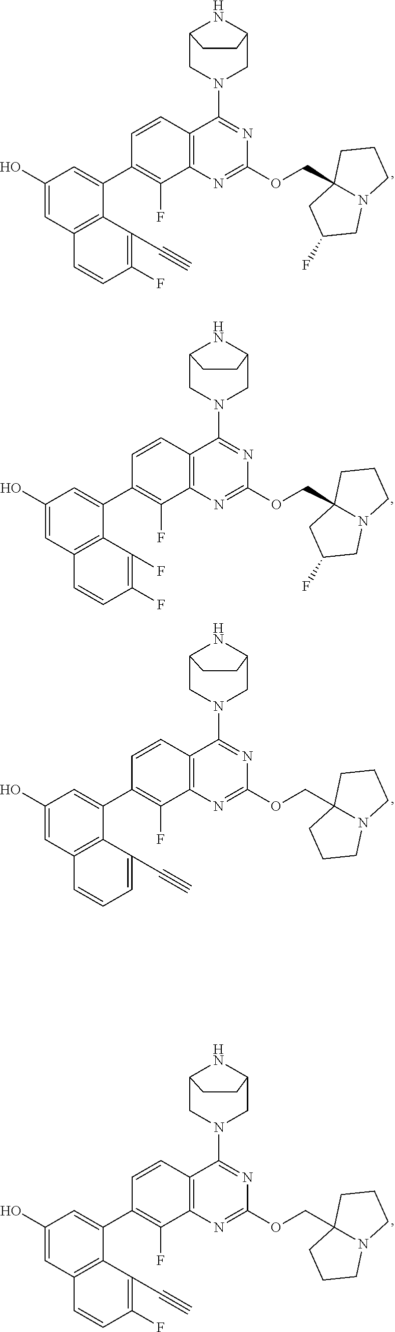 [SMILES: C#Cc1c(F)ccc2cc(O)cc(-c3ccc4c(N5CC6CCC(C5)N6)nc(OC[C@@]56CCCN5C[C@H](F)C6)nc4c3F)c12 ; Oc1cc(-c2ccc3c(N4CC5CCC(C4)N5)nc(OC[C@@]45CCCN4C[C@H](F)C5)nc3c2F)c2c(F)c(F)ccc2c1 ; C#Cc1cccc2cc(O)cc(-c3ccc4c(N5CC6CCC(C5)N6)nc(OCC56CCCN5CCC6)nc4c3F)c12 ; C#Cc1c(F)ccc2cc(O)cc(-c3ccc4c(N5CC6CCC(C5)N6)nc(OCC56CCCN5CCC6)nc4c3F)c12]

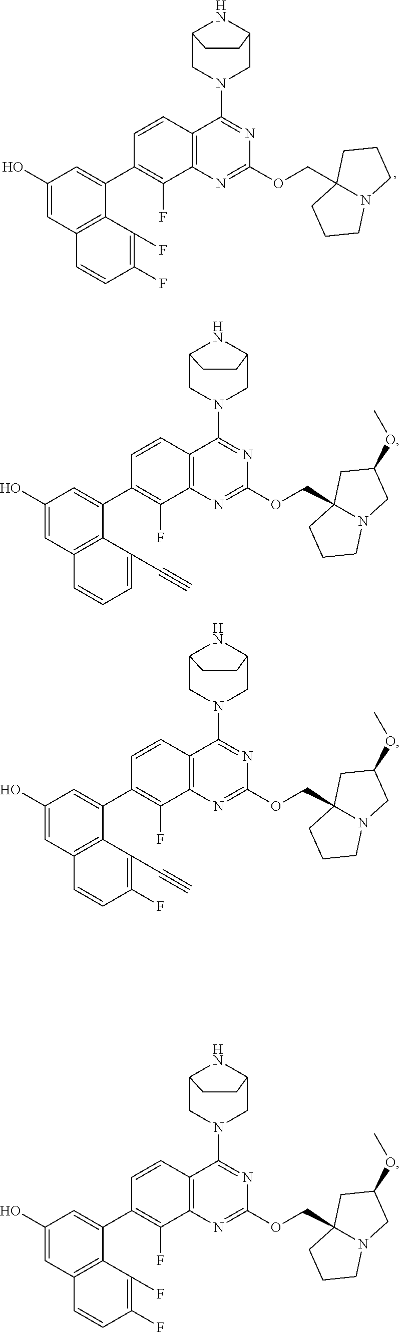 [SMILES: Oc1cc(-c2ccc3c(N4CC5CCC(C4)N5)nc(OCC45CCCN4CCC5)nc3c2F)c2c(F)c(F)ccc2c1 ; C#Cc1cccc2cc(O)cc(-c3ccc4c(N5CC6CCC(C5)N6)nc(OC[C@]56CCCN5C[C@H](OC)C6)nc4c3F)c12 ; C#Cc1c(F)ccc2cc(O)cc(-c3ccc4c(N5CC6CCC(C5)N6)nc(OC[C@]56CCCN5C[C@H](OC)C6)nc4c3F)c12 ; CO[C@H]1CN2CCC[C@]2(COc2nc(N3CC4CCC(C3)N4)c3ccc(-c4cc(O)cc5ccc(F)c(F)c45)c(F)c3n2)C1]

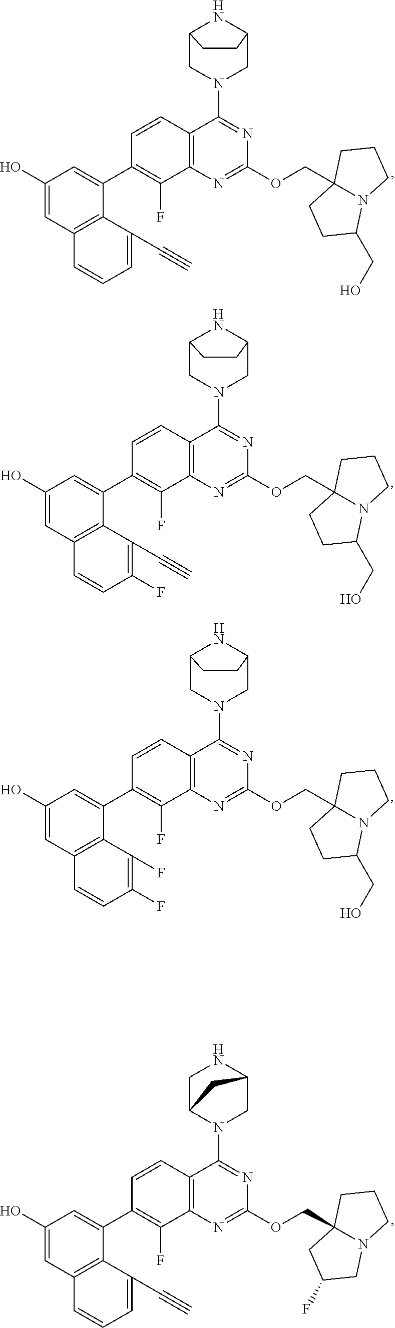 [SMILES: C#Cc1cccc2cc(O)cc(-c3ccc4c(N5CC6CCC(C5)N6)nc(OCC56CCCN5C(CO)CC6)nc4c3F)c12 ; C#Cc1c(F)ccc2cc(O)cc(-c3ccc4c(N5CC6CCC(C5)N6)nc(OCC56CCCN5C(CO)CC6)nc4c3F)c12 ; OCC1CCC2(COc3nc(N4CC5CCC(C4)N5)c4ccc(-c5cc(O)cc6ccc(F)c(F)c56)c(F)c4n3)CCCN12 ; C#Cc1cccc2cc(O)cc(-c3ccc4c(N5C[C@@H]6C[C@H]5CN6)nc(OC[C@@]56CCCN5C[C@H](F)C6)nc4c3F)c12]

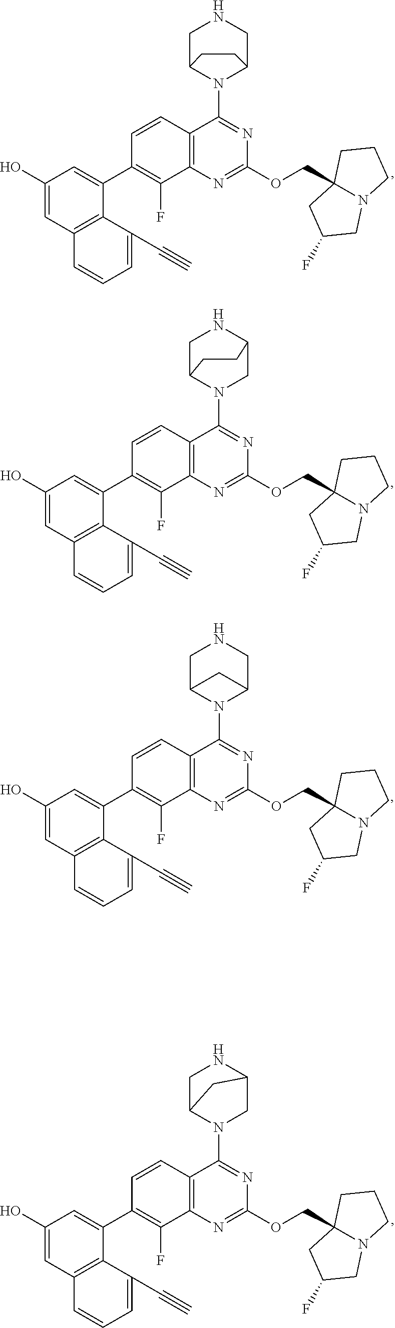 [SMILES: C#Cc1cccc2cc(O)cc(-c3ccc4c(N5C6CCC5CNC6)nc(OC[C@@]56CCCN5C[C@H](F)C6)nc4c3F)c12 ; C#Cc1cccc2cc(O)cc(-c3ccc4c(N5CC6CCC5CN6)nc(OC[C@@]56CCCN5C[C@H](F)C6)nc4c3F)c12 ; C#Cc1cccc2cc(O)cc(-c3ccc4c(N5C6CNCC5C6)nc(OC[C@@]56CCCN5C[C@H](F)C6)nc4c3F)c12 ; C#Cc1cccc2cc(O)cc(-c3ccc4c(N5CC6CC5CN6)nc(OC[C@@]56CCCN5C[C@H](F)C6)nc4c3F)c12]

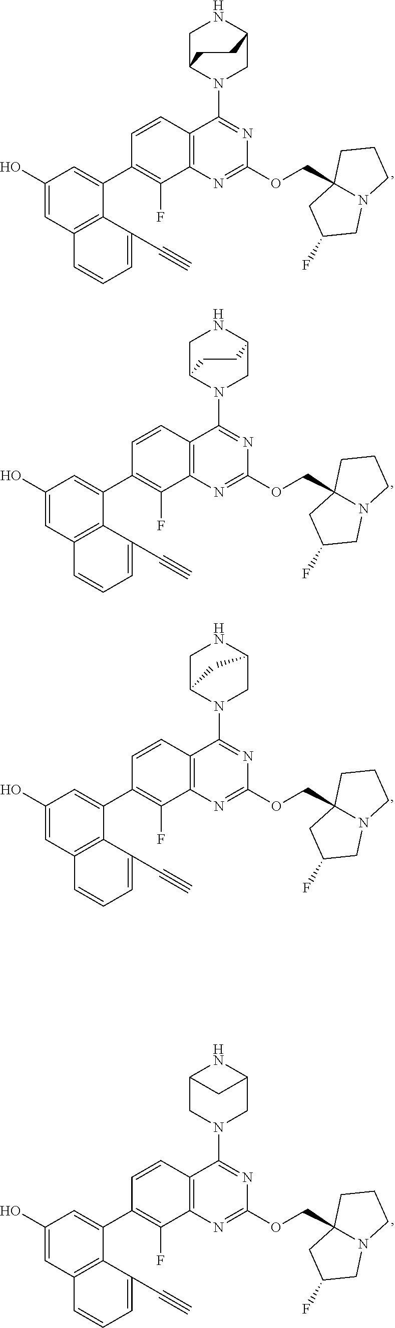 [SMILES: C#Cc1cccc2cc(O)cc(-c3ccc4c(N5C[C@@H]6CC[C@H]5CN6)nc(OC[C@@]56CCCN5C[C@H](F)C6)nc4c3F)c12 ; C#Cc1cccc2cc(O)cc(-c3ccc4c(N5C[C@H]6CC[C@@H]5CN6)nc(OC[C@@]56CCCN5C[C@H](F)C6)nc4c3F)c12 ; C#Cc1cccc2cc(O)cc(-c3ccc4c(N5C[C@H]6C[C@@H]5CN6)nc(OC[C@@]56CCCN5C[C@H](F)C6)nc4c3F)c12 ; C#Cc1cccc2cc(O)cc(-c3ccc4c(N5CC6CC(C5)N6)nc(OC[C@@]56CCCN5C[C@H](F)C6)nc4c3F)c12]

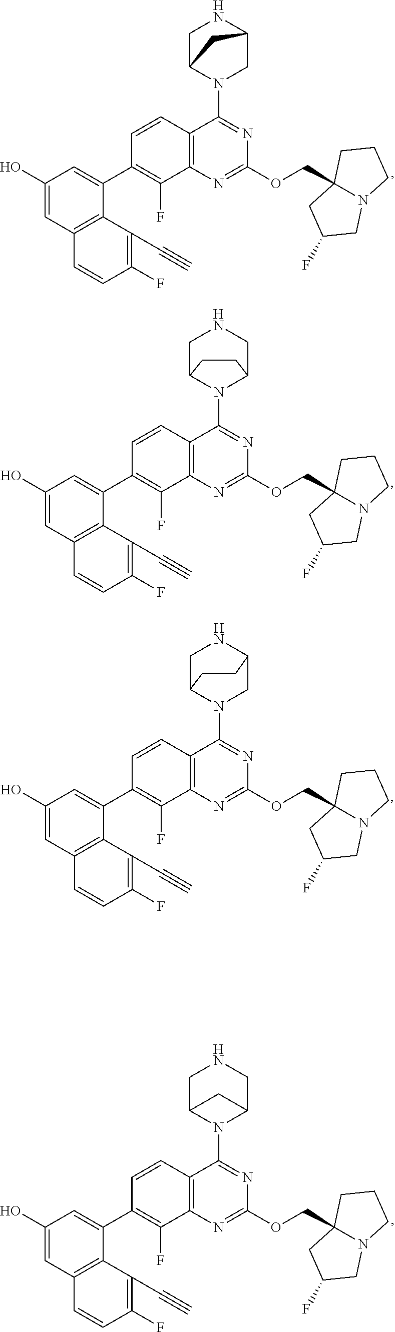 [SMILES: C#Cc1c(F)ccc2cc(O)cc(-c3ccc4c(N5C[C@@H]6C[C@H]5CN6)nc(OC[C@@]56CCCN5C[C@H](F)C6)nc4c3F)c12 ; C#Cc1c(F)ccc2cc(O)cc(-c3ccc4c(N5C6CCC5CNC6)nc(OC[C@@]56CCCN5C[C@H](F)C6)nc4c3F)c12 ; C#Cc1c(F)ccc2cc(O)cc(-c3ccc4c(N5CC6CCC5CN6)nc(OC[C@@]56CCCN5C[C@H](F)C6)nc4c3F)c12 ; C#Cc1c(F)ccc2cc(O)cc(-c3ccc4c(N5C6CNCC5C6)nc(OC[C@@]56CCCN5C[C@H](F)C6)nc4c3F)c12]

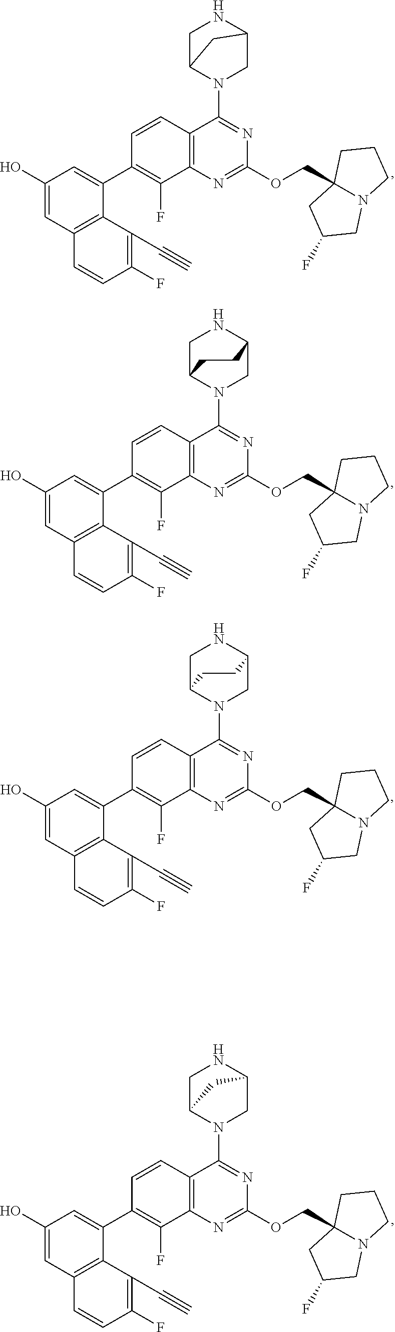 [SMILES: C#Cc1c(F)ccc2cc(O)cc(-c3ccc4c(N5CC6CC5CN6)nc(OC[C@@]56CCCN5C[C@H](F)C6)nc4c3F)c12 ; C#Cc1c(F)ccc2cc(O)cc(-c3ccc4c(N5C[C@@H]6CC[C@H]5CN6)nc(OC[C@@]56CCCN5C[C@H](F)C6)nc4c3F)c12 ; C#Cc1c(F)ccc2cc(O)cc(-c3ccc4c(N5C[C@H]6CC[C@@H]5CN6)nc(OC[C@@]56CCCN5C[C@H](F)C6)nc4c3F)c12 ; C#Cc1c(F)ccc2cc(O)cc(-c3ccc4c(N5C[C@H]6C[C@@H]5CN6)nc(OC[C@@]56CCCN5C[C@H](F)C6)nc4c3F)c12]

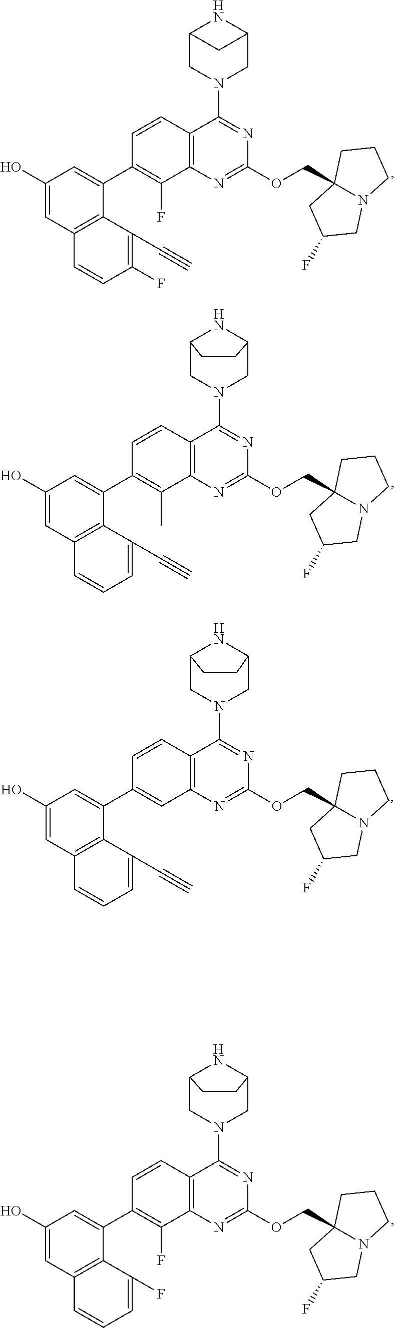 [SMILES: C#Cc1c(F)ccc2cc(O)cc(-c3ccc4c(N5CC6CC(C5)N6)nc(OC[C@@]56CCCN5C[C@H](F)C6)nc4c3F)c12 ; C#Cc1cccc2cc(O)cc(-c3ccc4c(N5CC6CCC(C5)N6)nc(OC[C@@]56CCCN5C[C@H](F)C6)nc4c3C)c12 ; C#Cc1cccc2cc(O)cc(-c3ccc4c(N5CC6CCC(C5)N6)nc(OC[C@@]56CCCN5C[C@H](F)C6)nc4c3)c12 ; Oc1cc(-c2ccc3c(N4CC5CCC(C4)N5)nc(OC[C@@]45CCCN4C[C@H](F)C5)nc3c2F)c2c(F)cccc2c1]

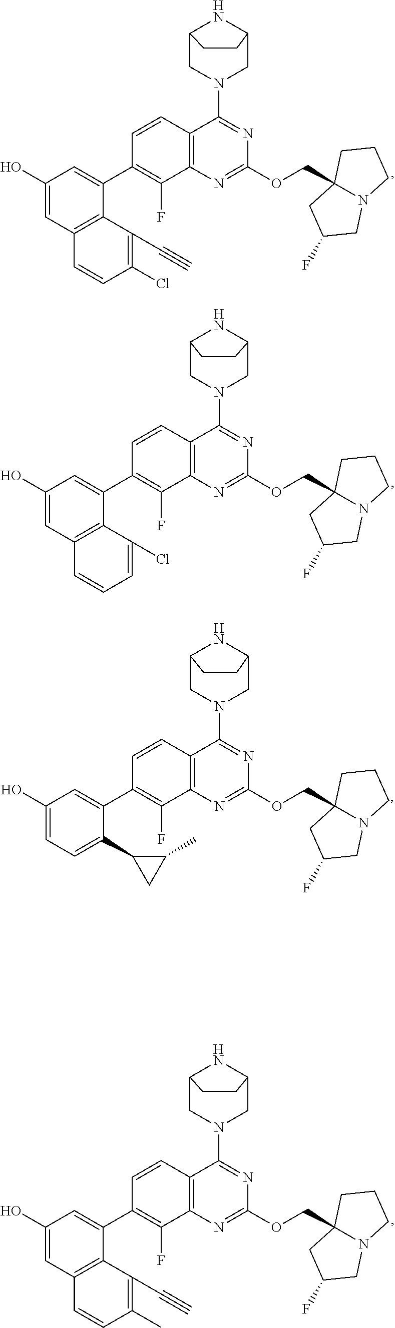 [SMILES: C#Cc1c(Cl)ccc2cc(O)cc(-c3ccc4c(N5CC6CCC(C5)N6)nc(OC[C@@]56CCCN5C[C@H](F)C6)nc4c3F)c12 ; Oc1cc(-c2ccc3c(N4CC5CCC(C4)N5)nc(OC[C@@]45CCCN4C[C@H](F)C5)nc3c2F)c2c(Cl)cccc2c1 ; C[C@@H]1C[C@H]1c1ccc(O)cc1-c1ccc2c(N3CC4CCC(C3)N4)nc(OC[C@@]34CCCN3C[C@H](F)C4)nc2c1F ; C#Cc1c(C)ccc2cc(O)cc(-c3ccc4c(N5CC6CCC(C5)N6)nc(OC[C@@]56CCCN5C[C@H](F)C6)nc4c3F)c12]

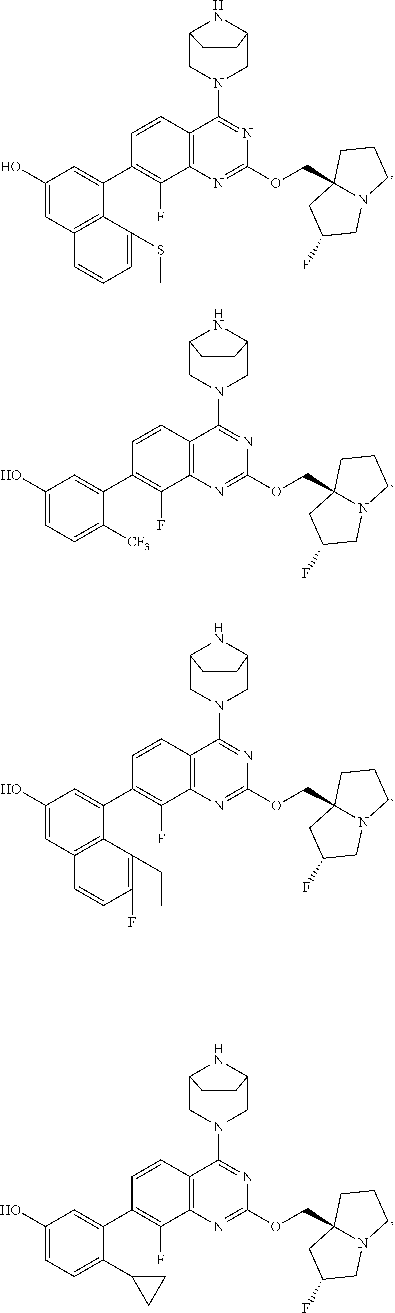 [SMILES: CSc1cccc2cc(O)cc(-c3ccc4c(N5CC6CCC(C5)N6)nc(OC[C@@]56CCCN5C[C@H](F)C6)nc4c3F)c12 ; Oc1ccc(C(F)(F)F)c(-c2ccc3c(N4CC5CCC(C4)N5)nc(OC[C@@]45CCCN4C[C@H](F)C5)nc3c2F)c1 ; CCc1c(F)ccc2cc(O)cc(-c3ccc4c(N5CC6CCC(C5)N6)nc(OC[C@@]56CCCN5C[C@H](F)C6)nc4c3F)c12 ; Oc1ccc(C2CC2)c(-c2ccc3c(N4CC5CCC(C4)N5)nc(OC[C@@]45CCCN4C[C@H](F)C5)nc3c2F)c1]

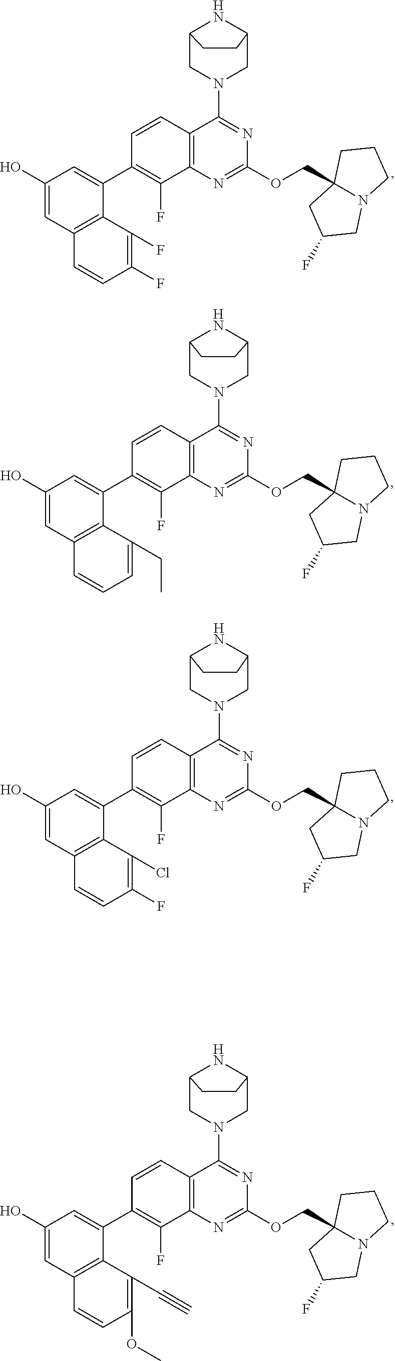 [SMILES: Oc1cc(-c2ccc3c(N4CC5CCC(C4)N5)nc(OC[C@@]45CCCN4C[C@H](F)C5)nc3c2F)c2c(F)c(F)ccc2c1 ; CCc1cccc2cc(O)cc(-c3ccc4c(N5CC6CCC(C5)N6)nc(OC[C@@]56CCCN5C[C@H](F)C6)nc4c3F)c12 ; Oc1cc(-c2ccc3c(N4CC5CCC(C4)N5)nc(OC[C@@]45CCCN4C[C@H](F)C5)nc3c2F)c2c(Cl)c(F)ccc2c1 ; C#Cc1c(OC)ccc2cc(O)cc(-c3ccc4c(N5CC6CCC(C5)N6)nc(OC[C@@]56CCCN5C[C@H](F)C6)nc4c3F)c12]

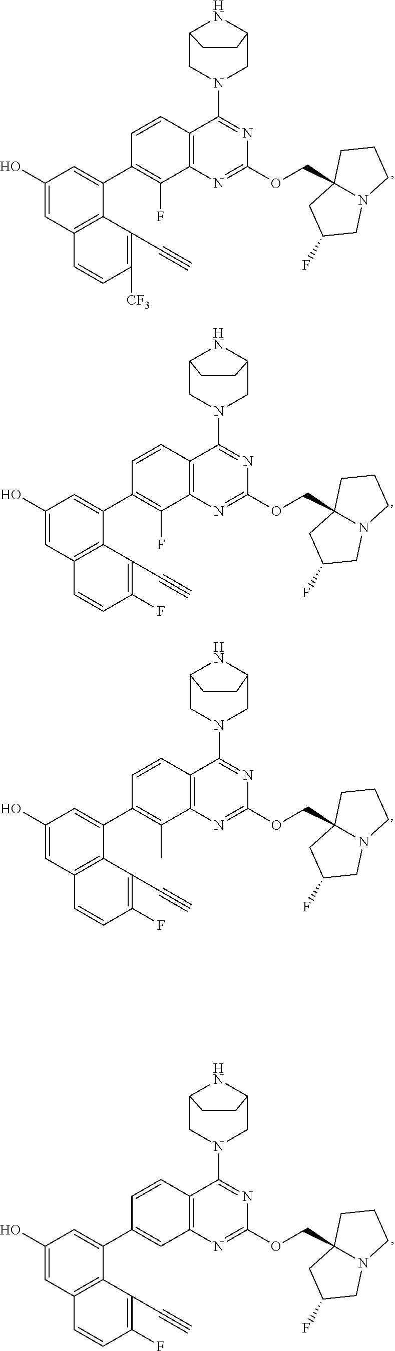 [SMILES: C#Cc1c(C(F)(F)F)ccc2cc(O)cc(-c3ccc4c(N5CC6CCC(C5)N6)nc(OC[C@@]56CCCN5C[C@H](F)C6)nc4c3F)c12 ; C#Cc1c(F)ccc2cc(O)cc(-c3ccc4c(N5CC6CCC(C5)N6)nc(OC[C@@]56CCCN5C[C@H](F)C6)nc4c3F)c12 ; C#Cc1c(F)ccc2cc(O)cc(-c3ccc4c(N5CC6CCC(C5)N6)nc(OC[C@@]56CCCN5C[C@H](F)C6)nc4c3C)c12 ; C#Cc1c(F)ccc2cc(O)cc(-c3ccc4c(N5CC6CCC(C5)N6)nc(OC[C@@]56CCCN5C[C@H](F)C6)nc4c3)c12]

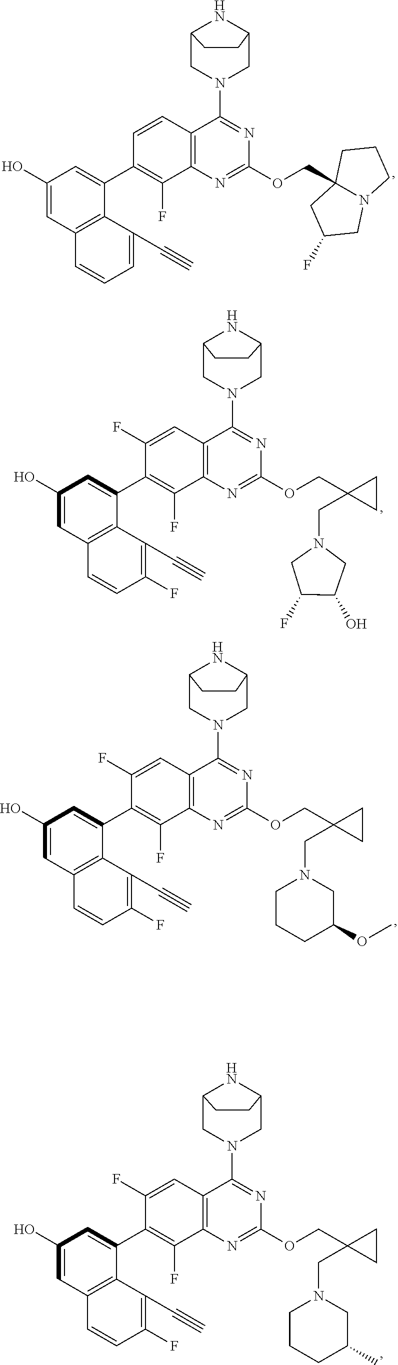

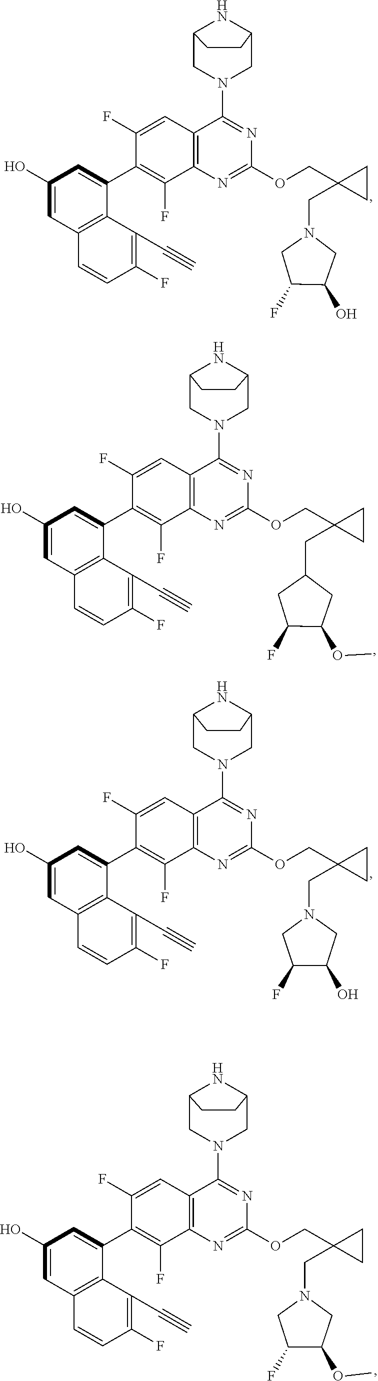

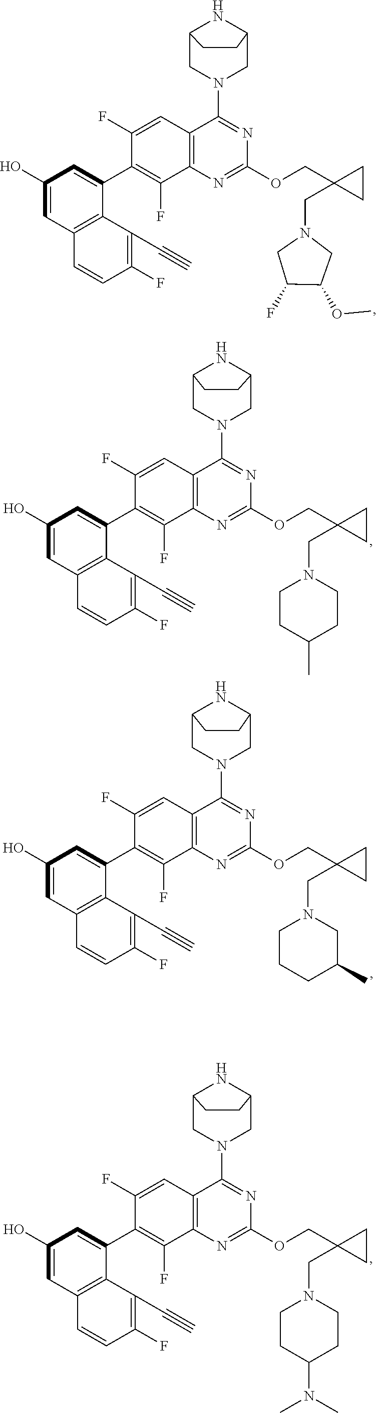

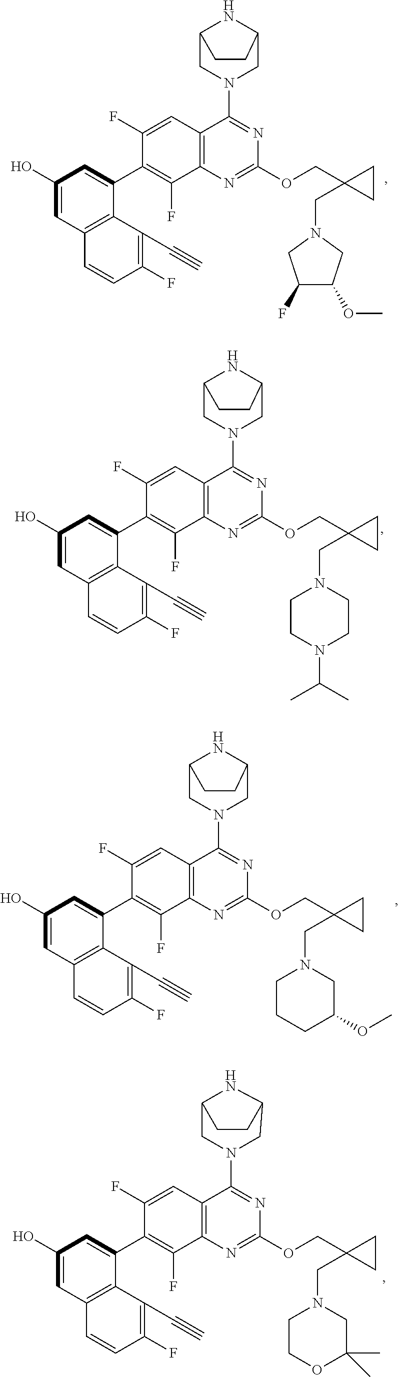

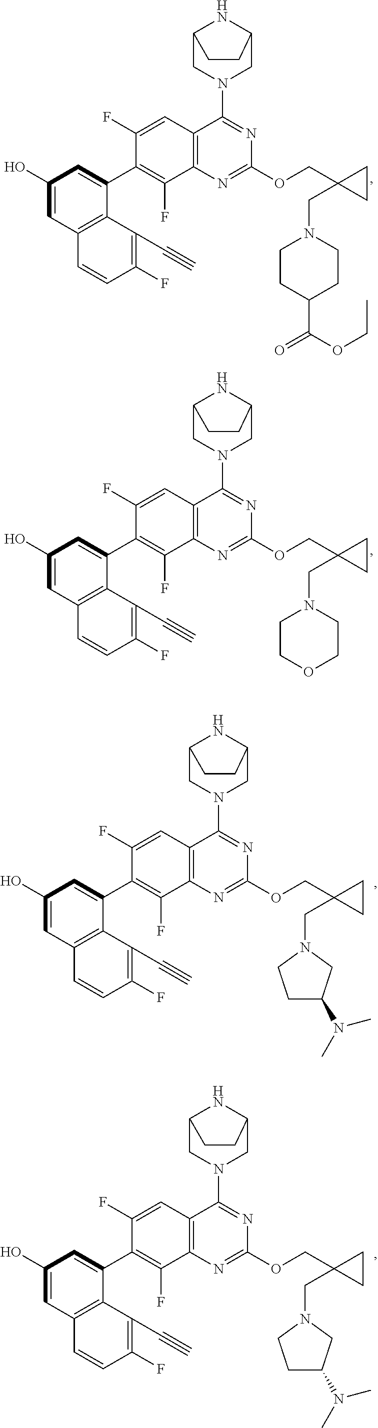

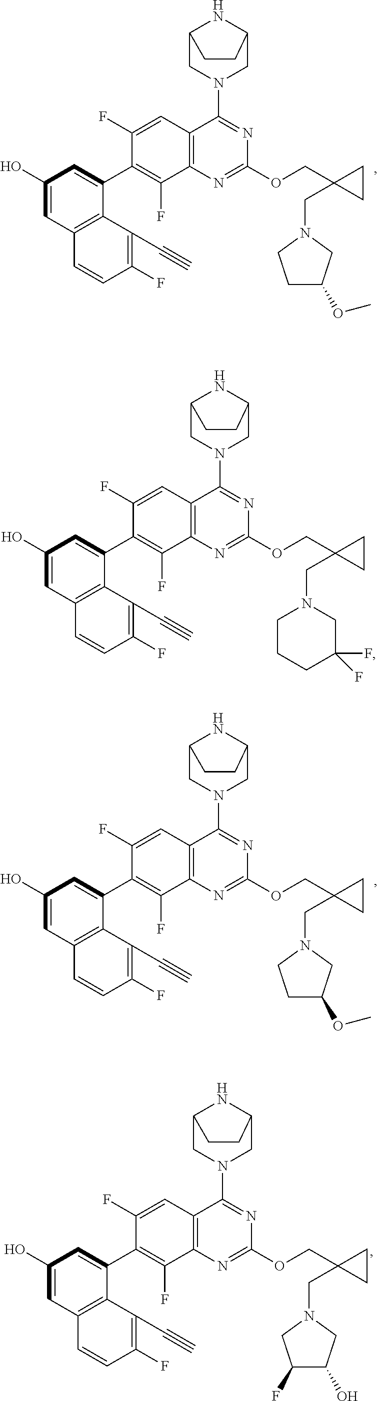

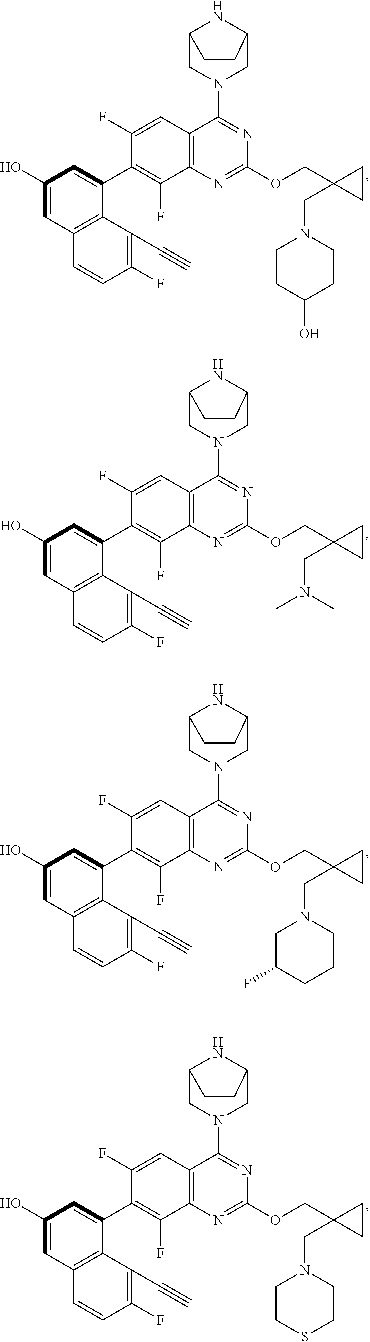

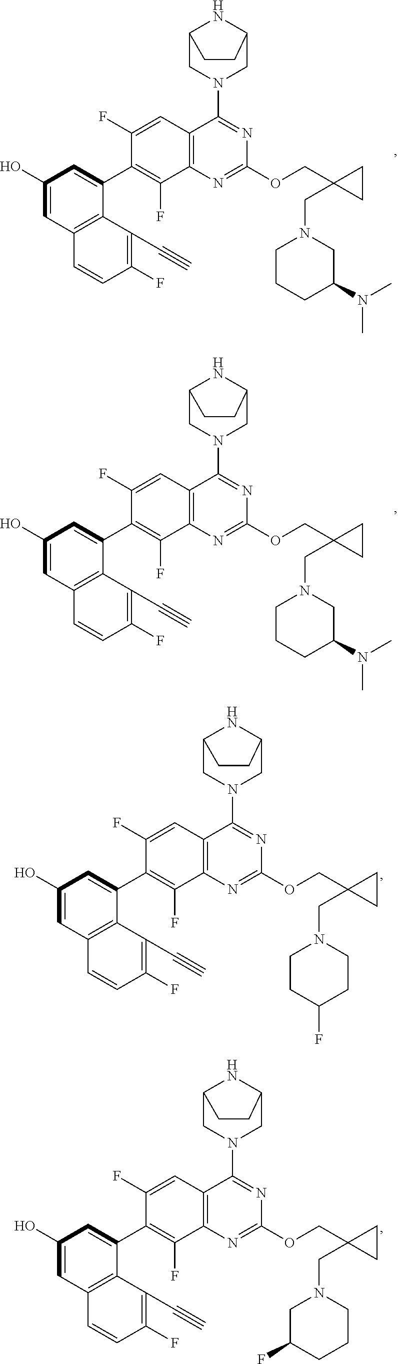

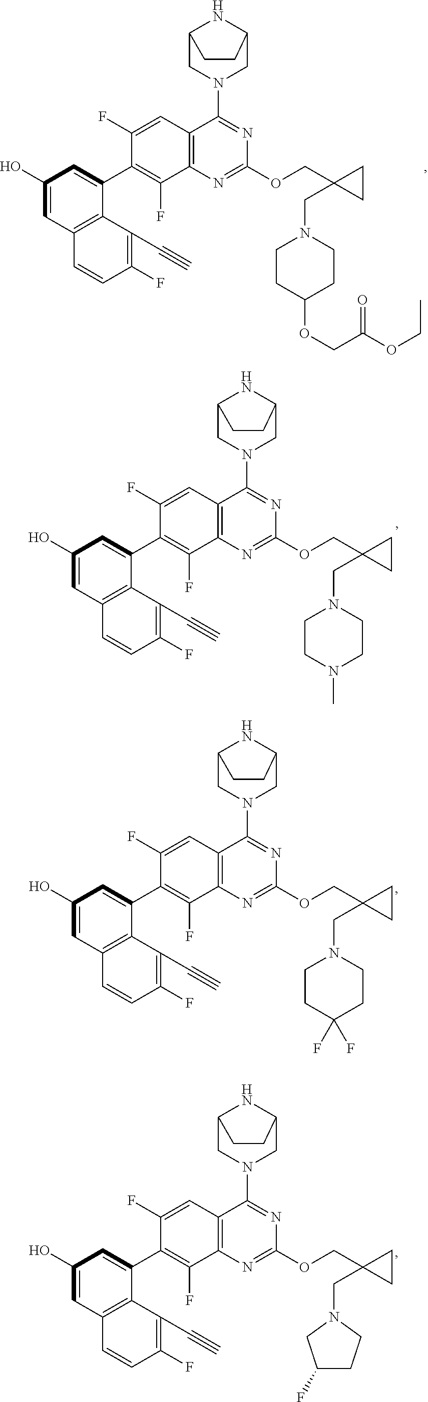

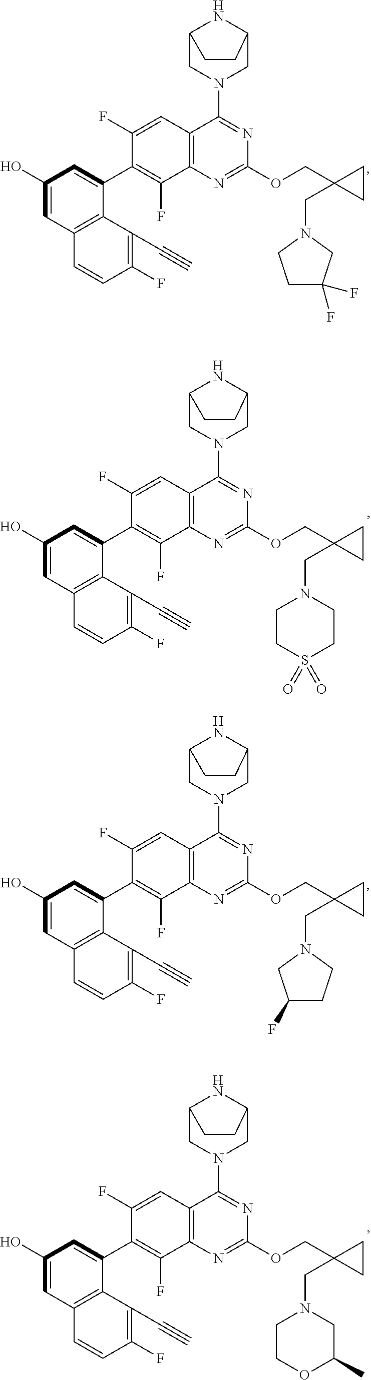

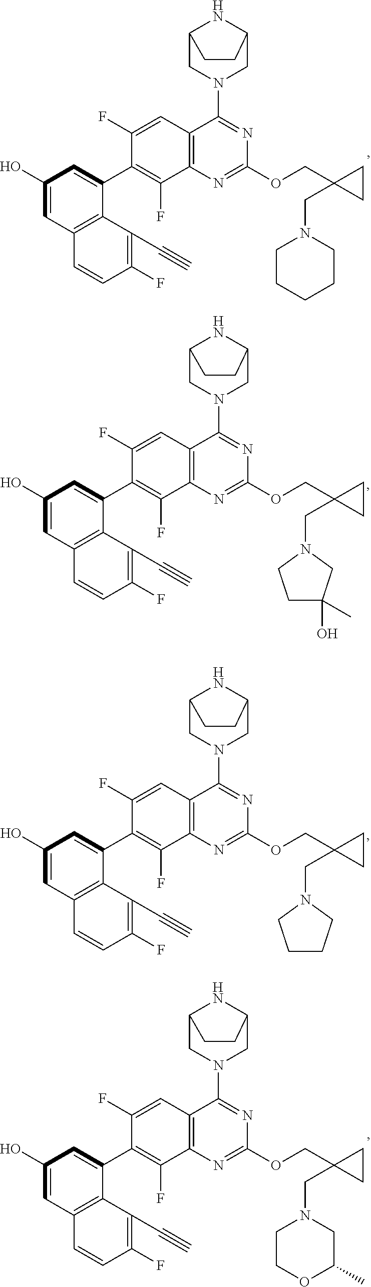

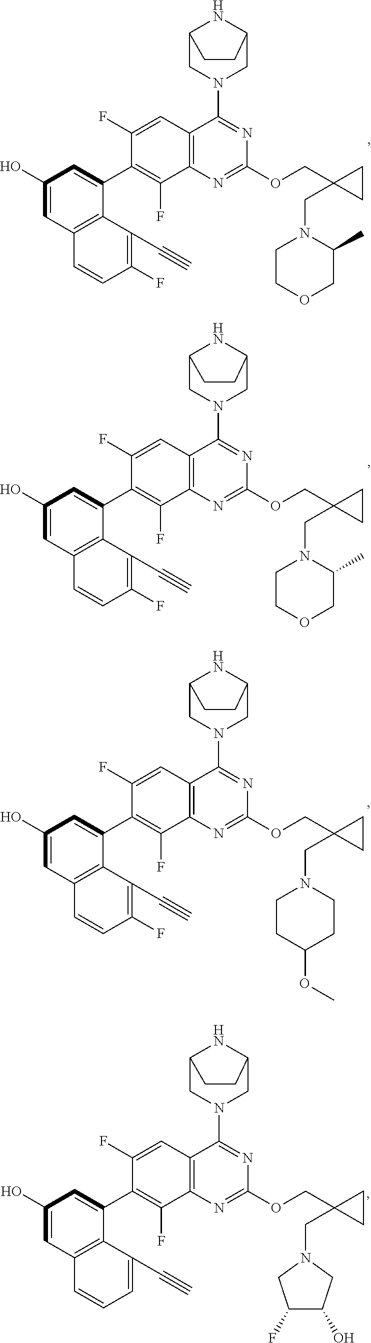

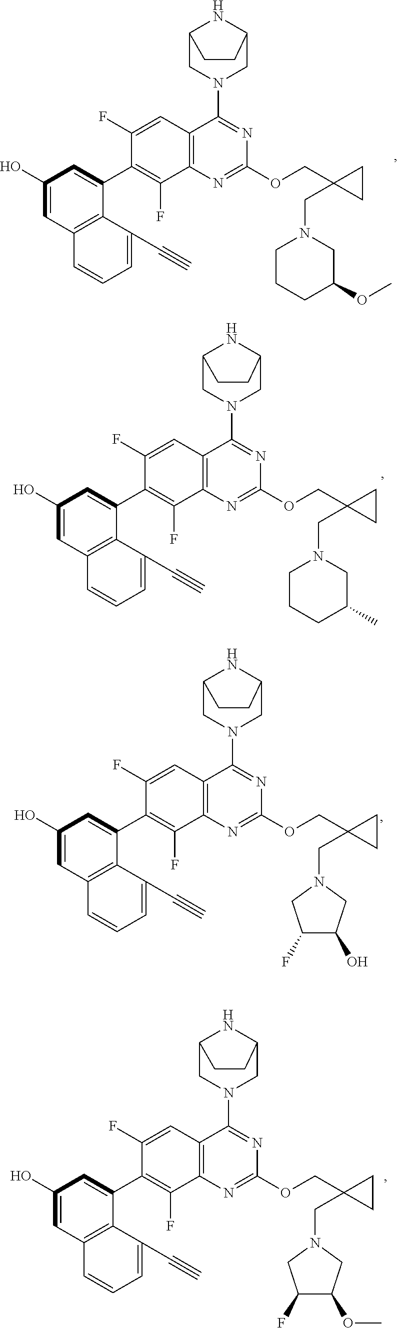

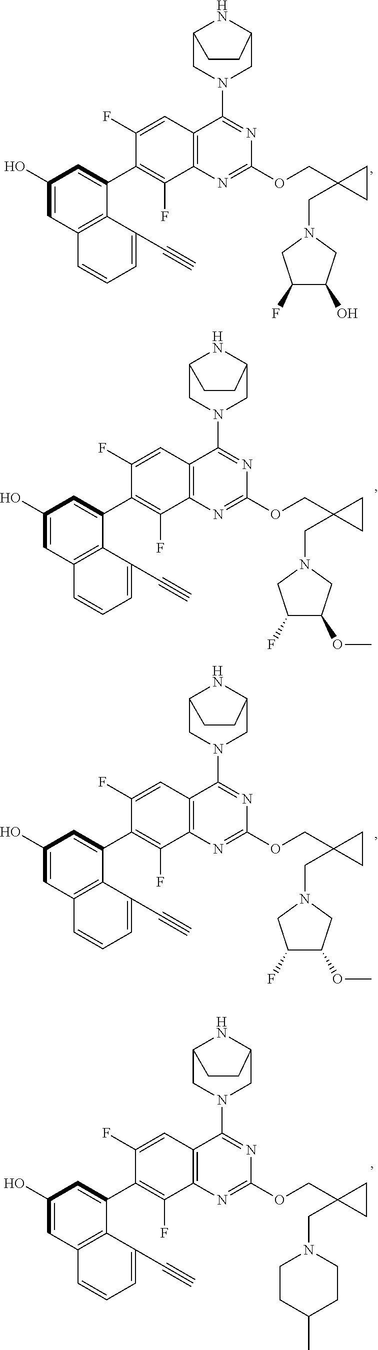

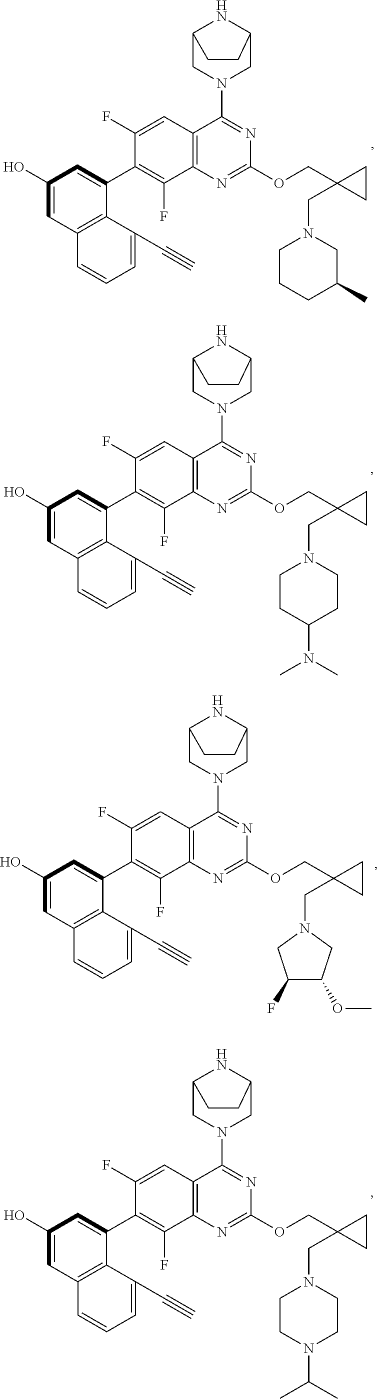

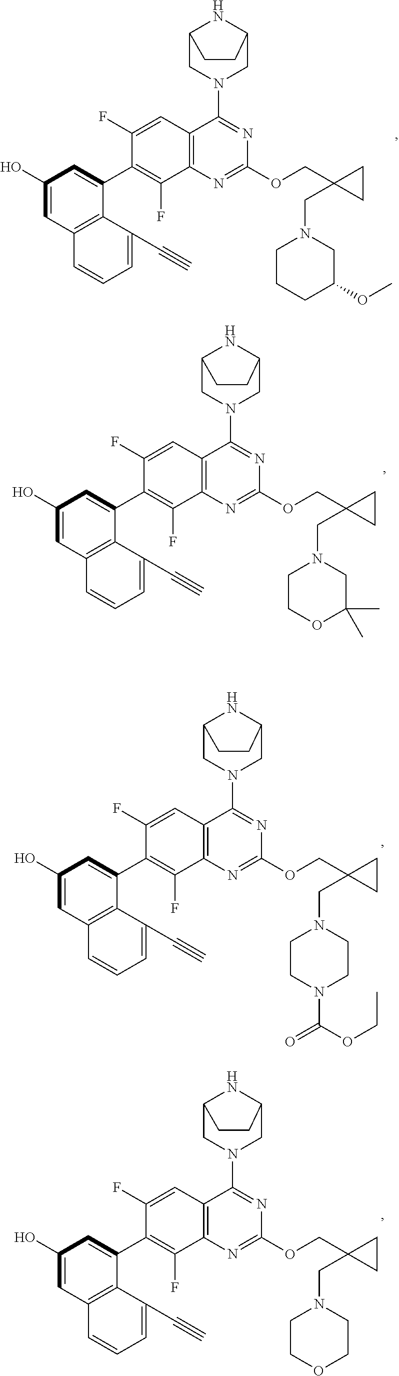

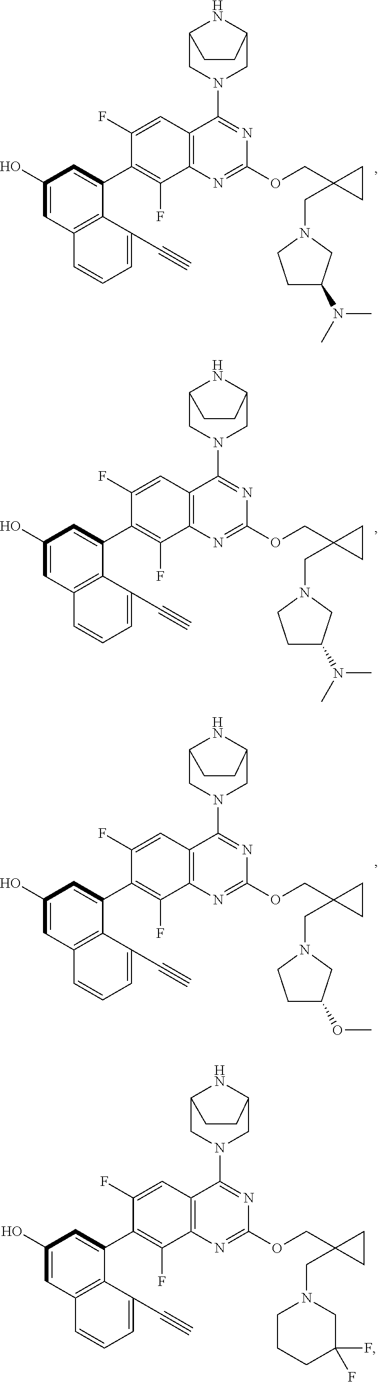

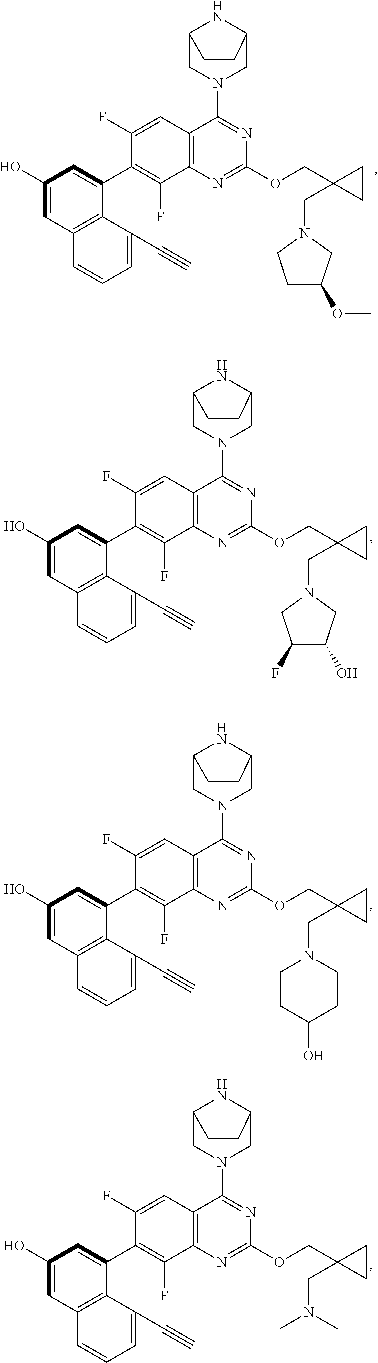

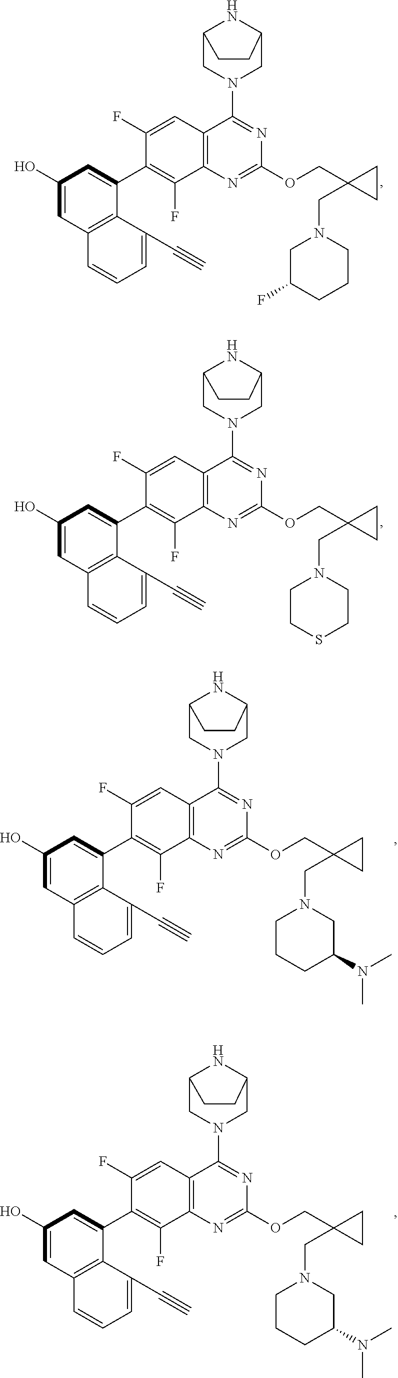

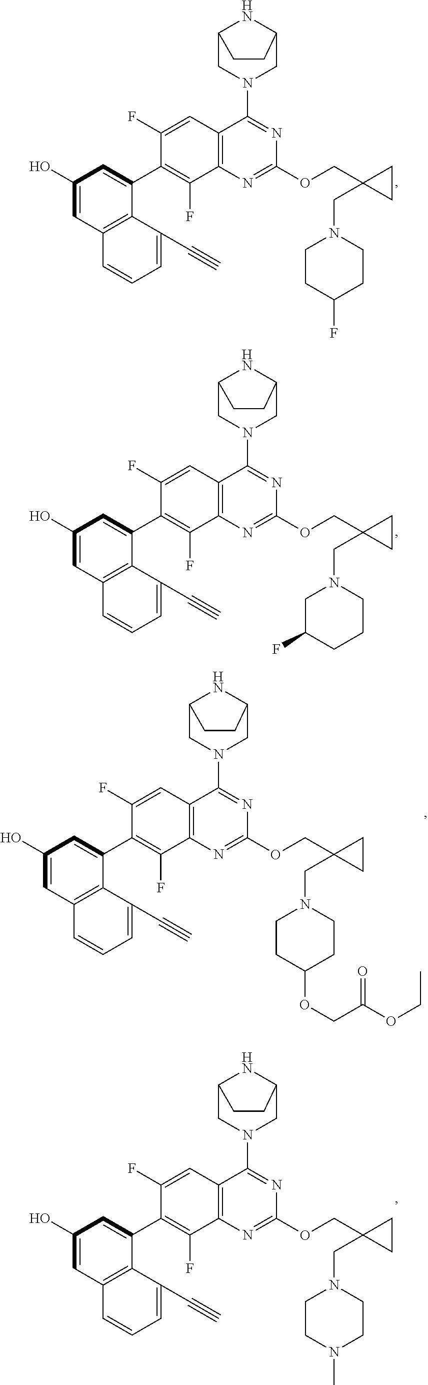

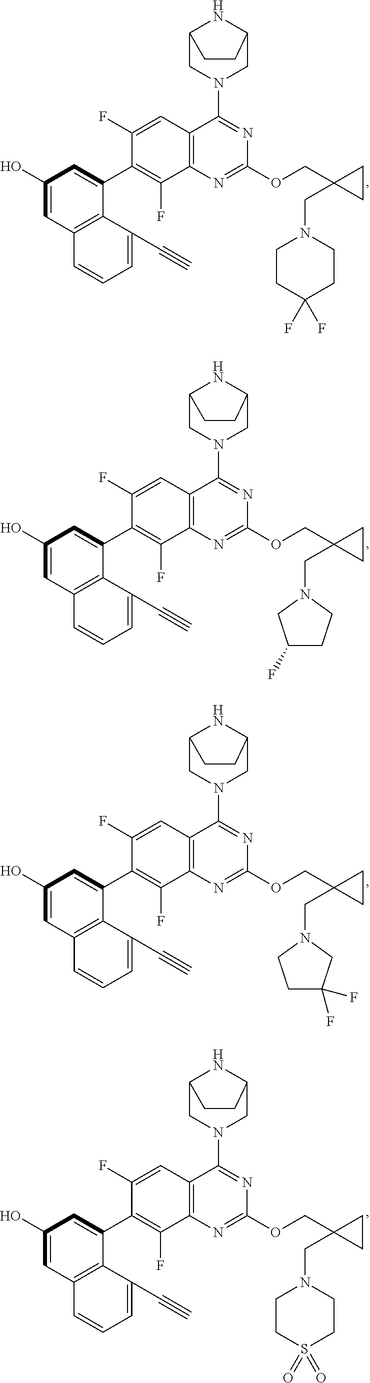

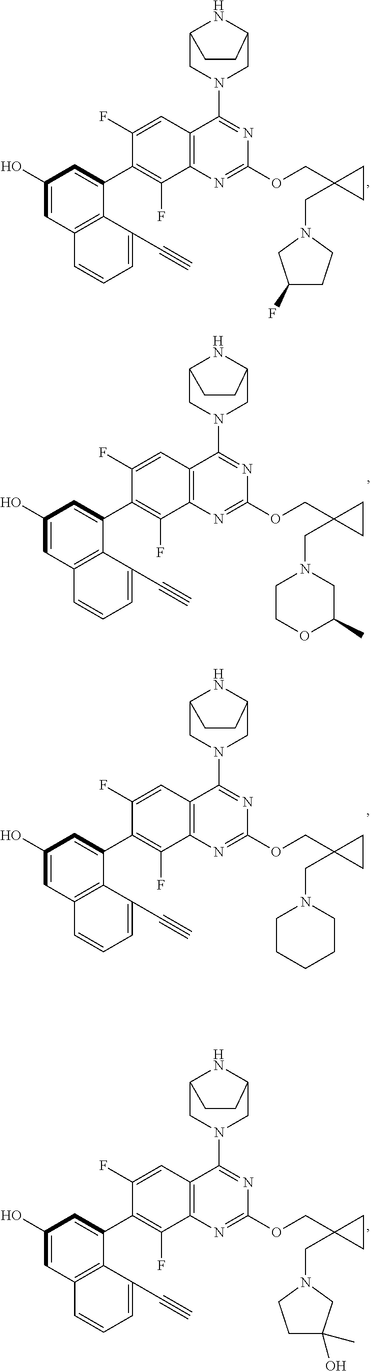

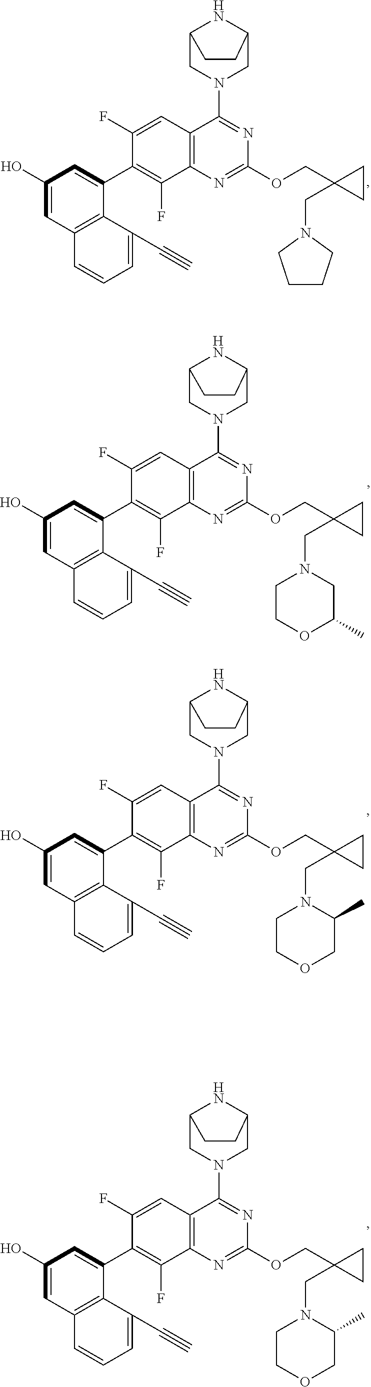

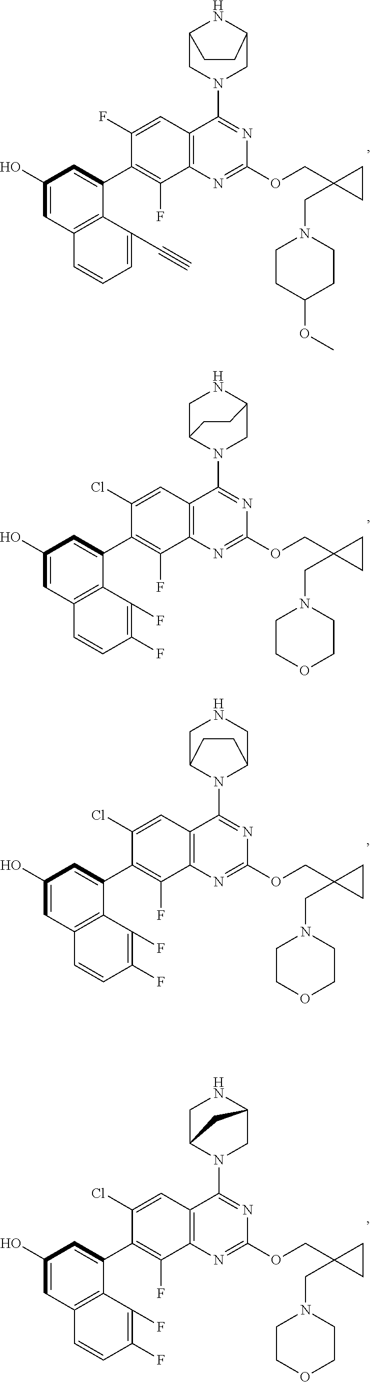

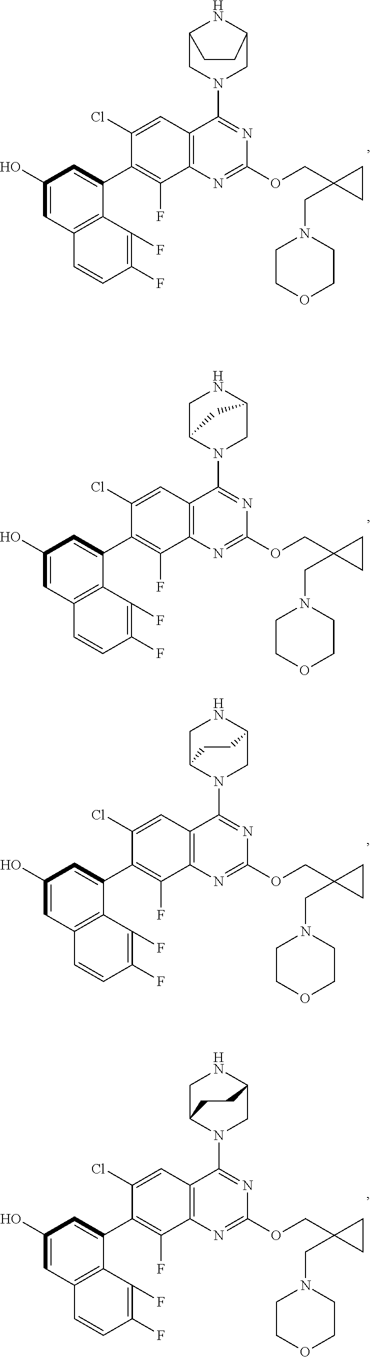

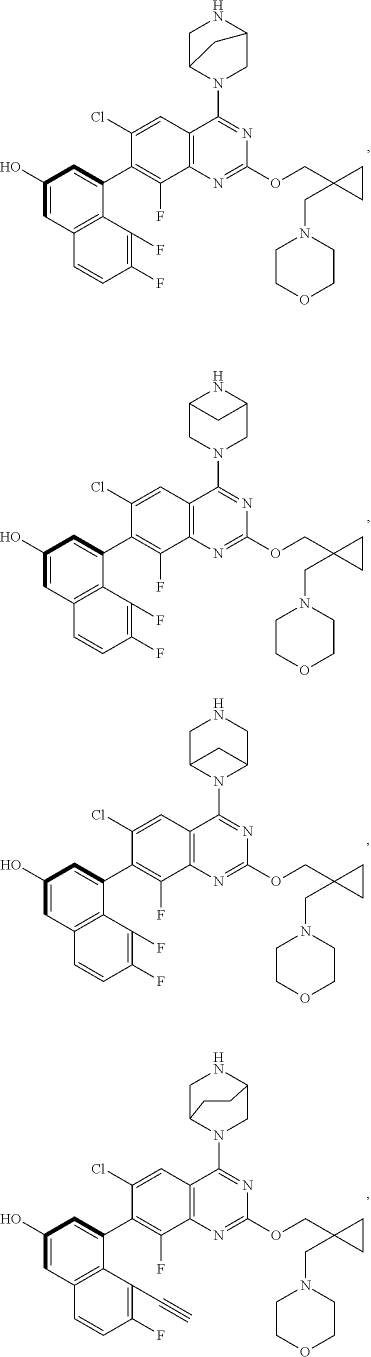

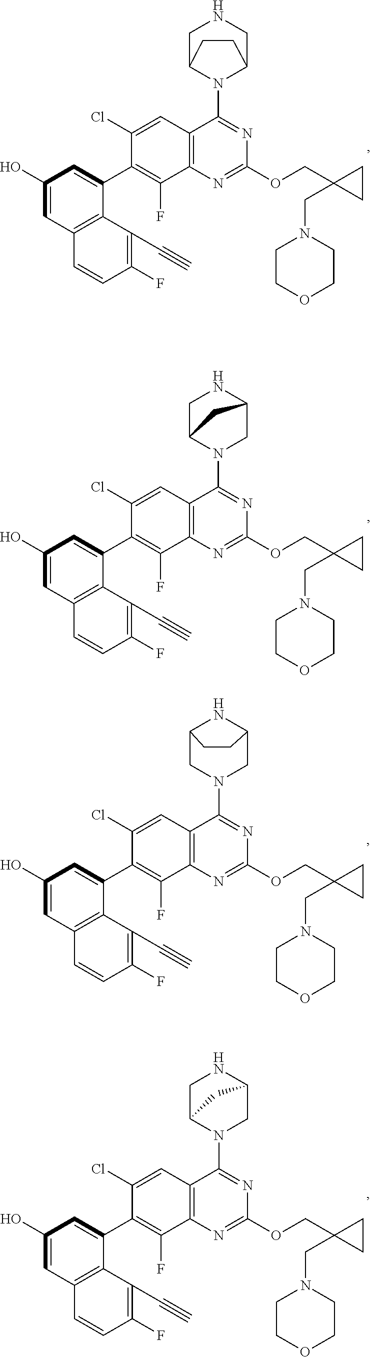

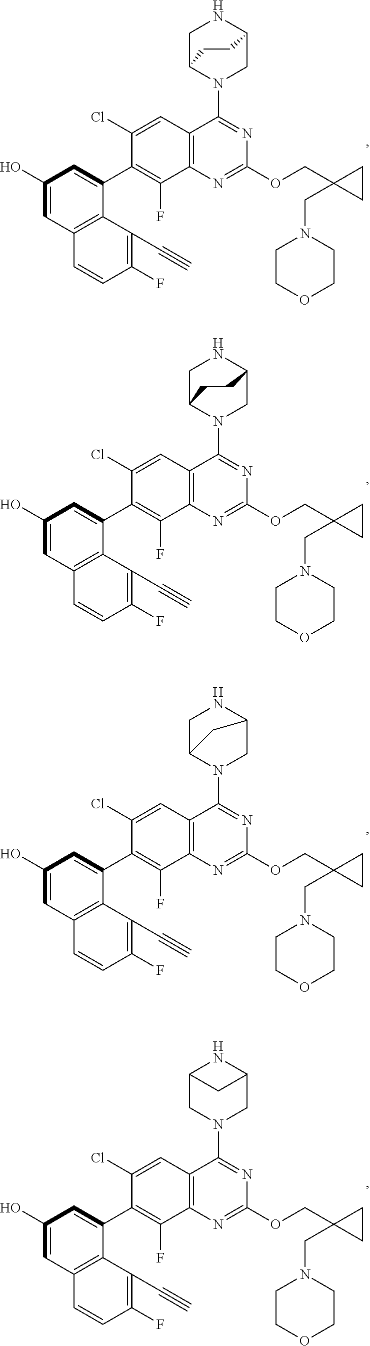

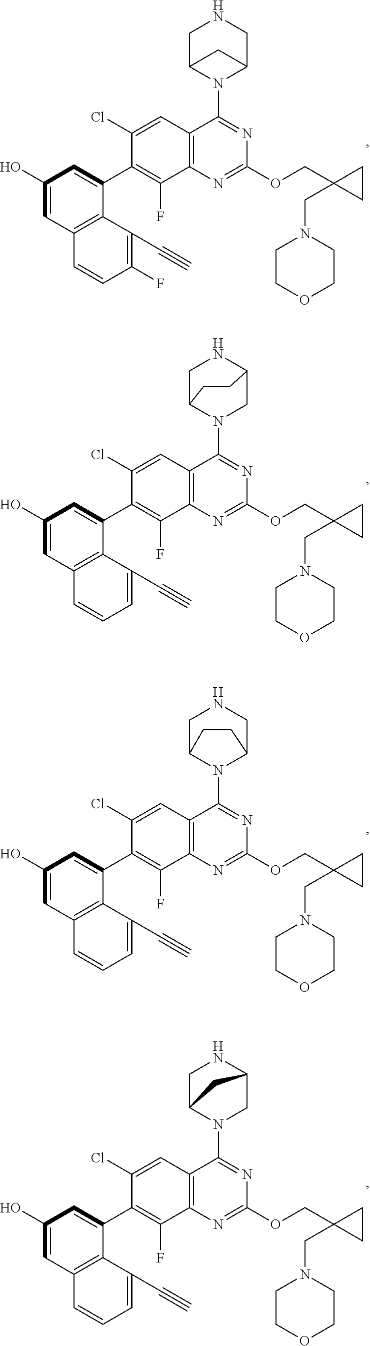

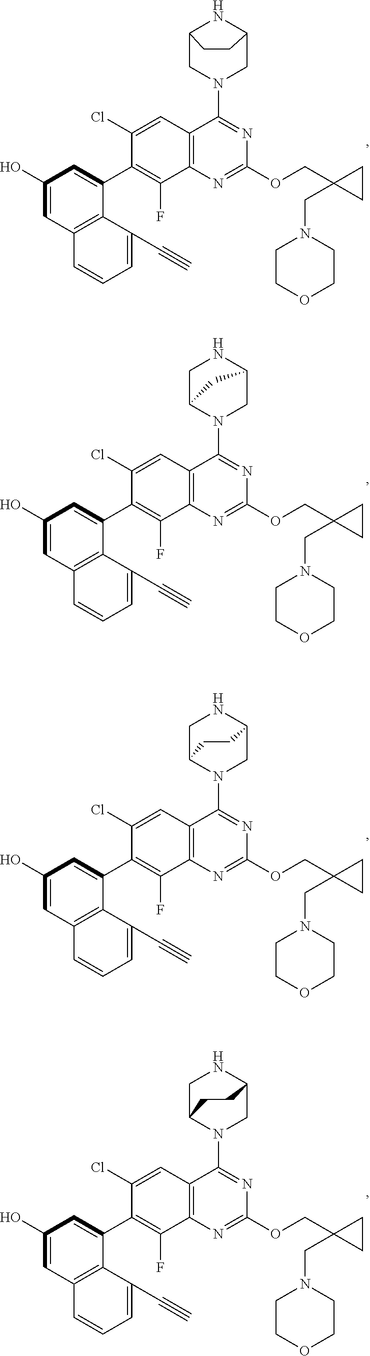

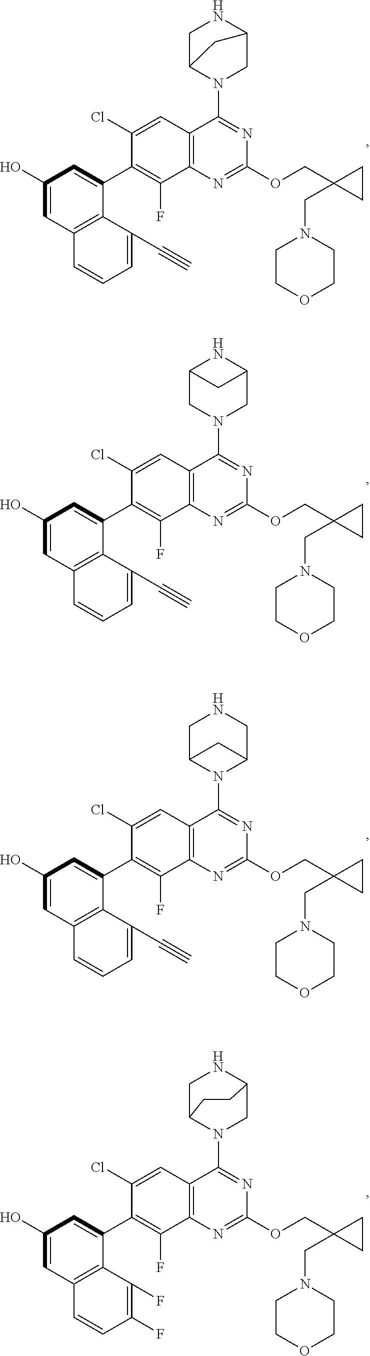

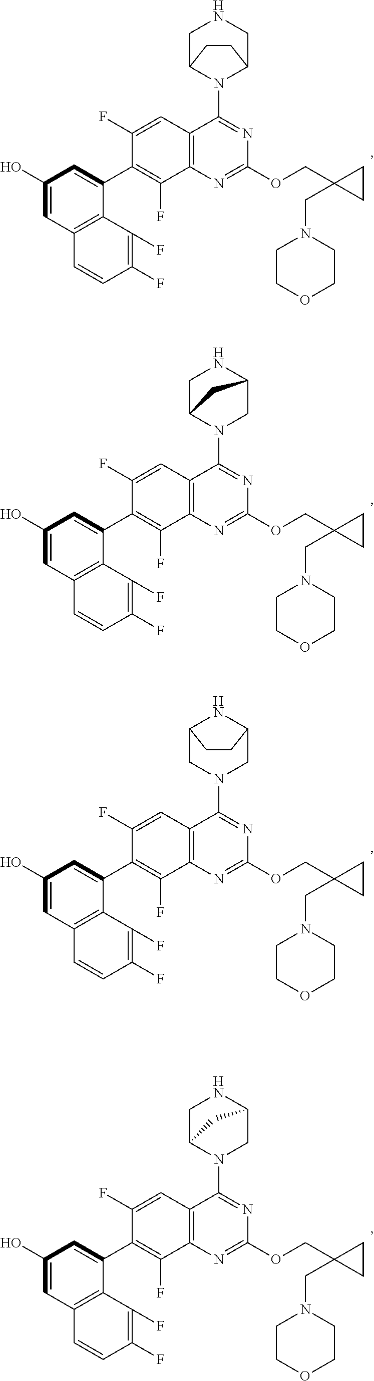

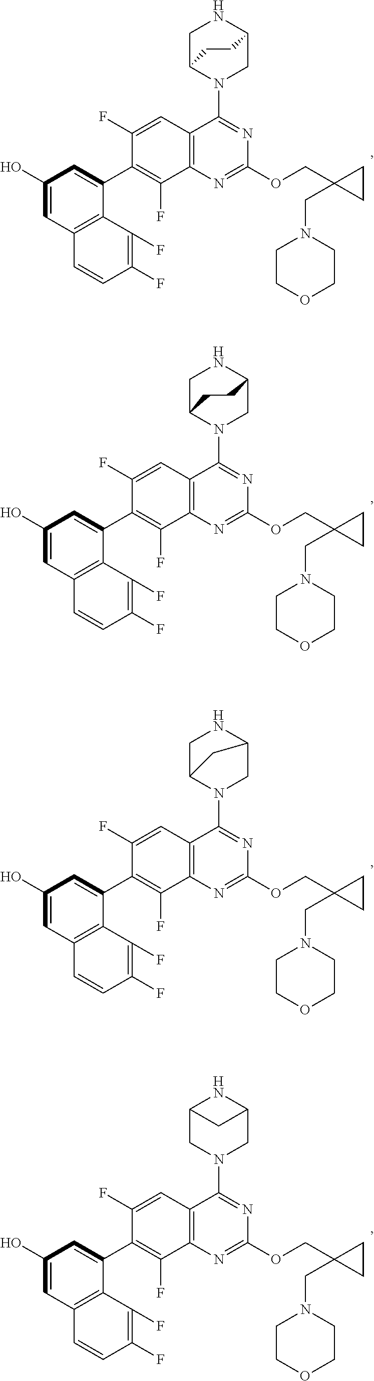

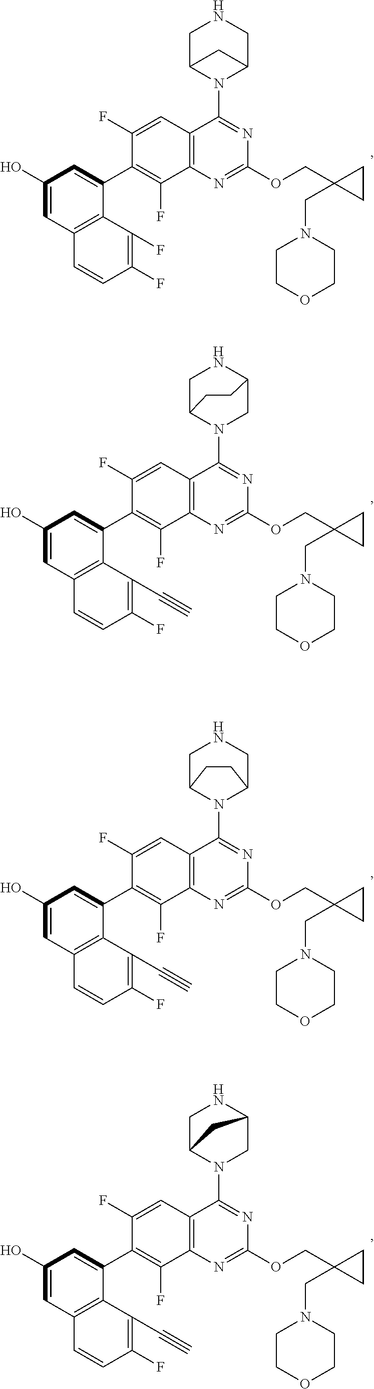

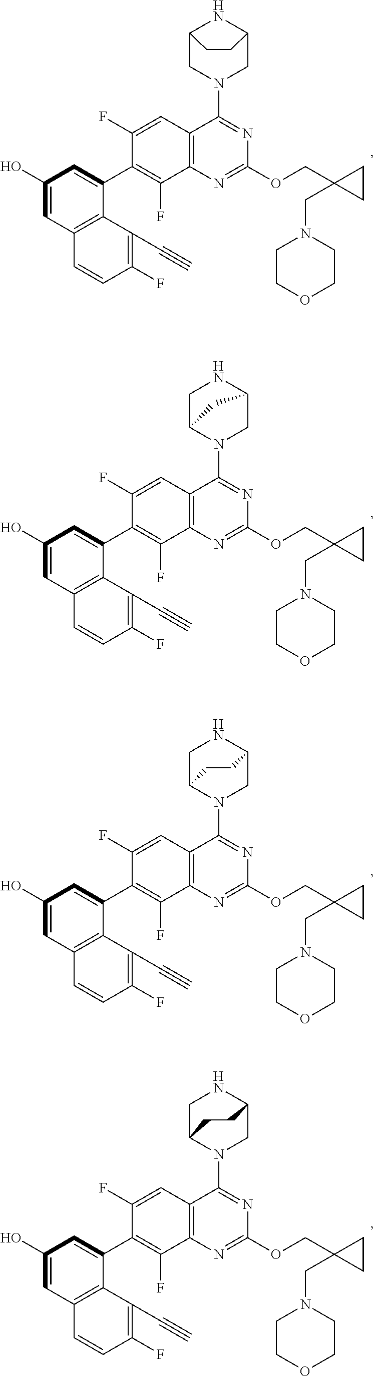

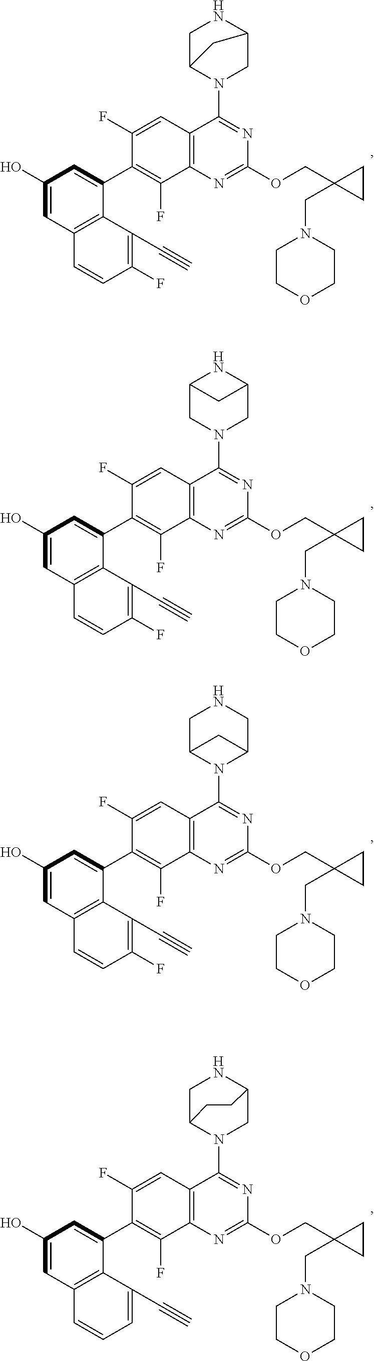

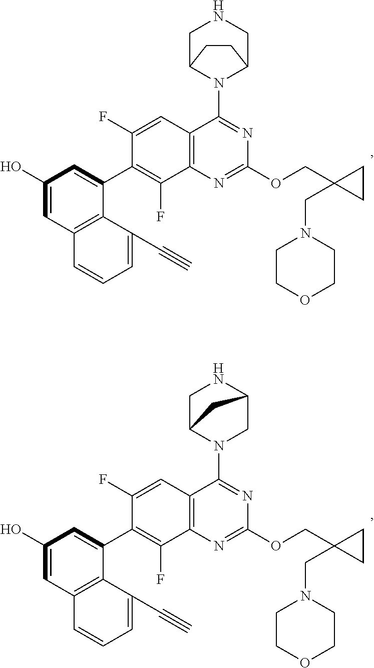

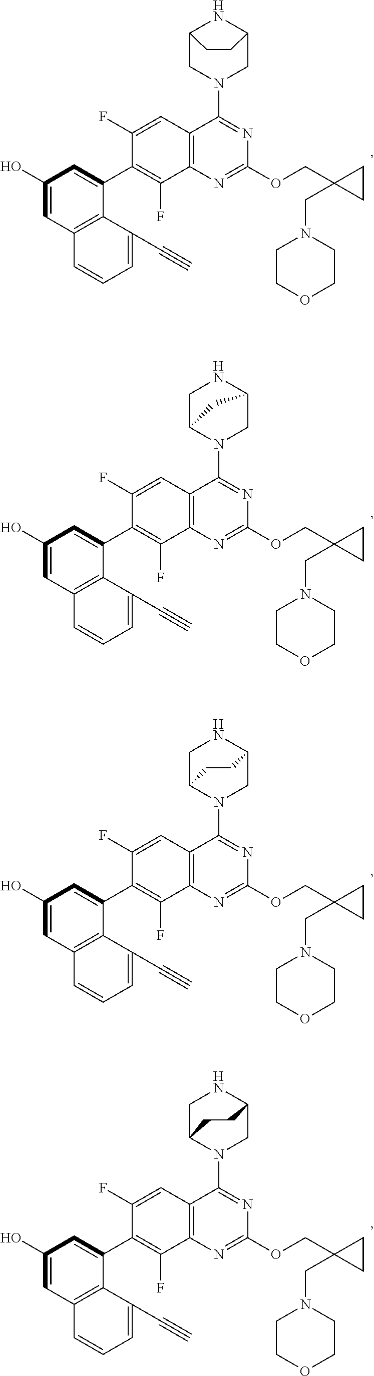

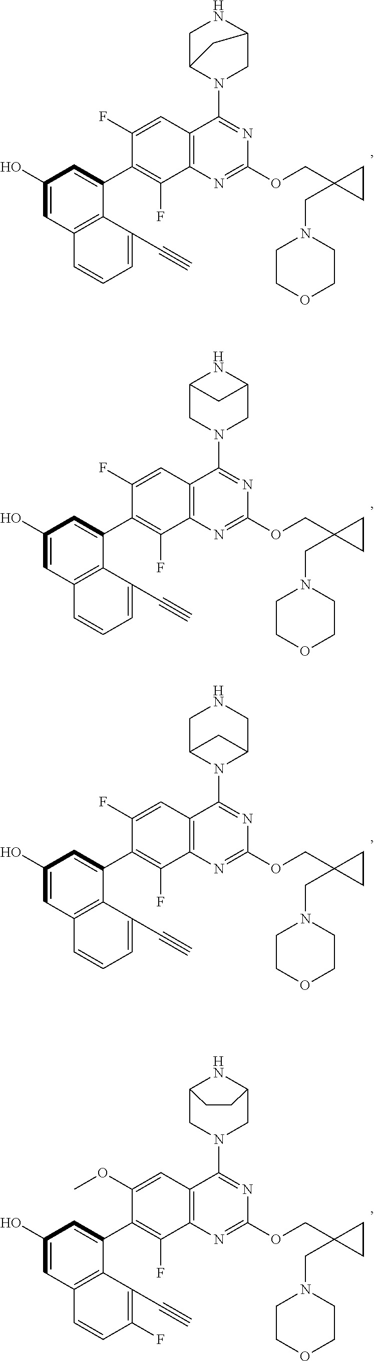

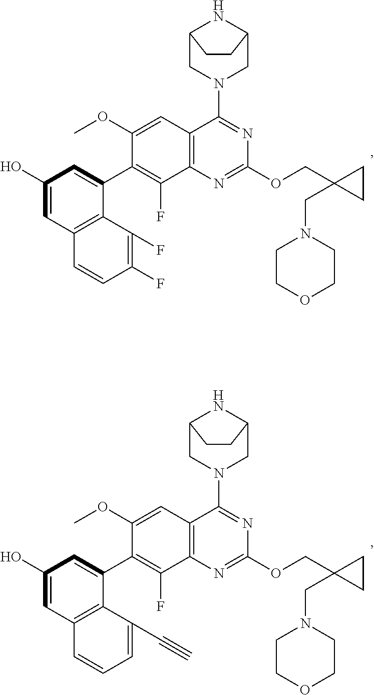

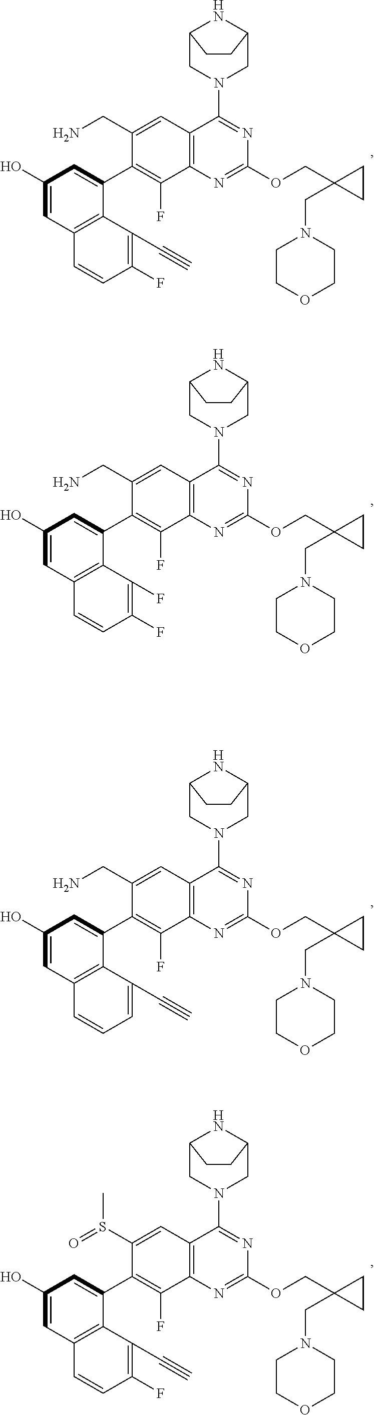

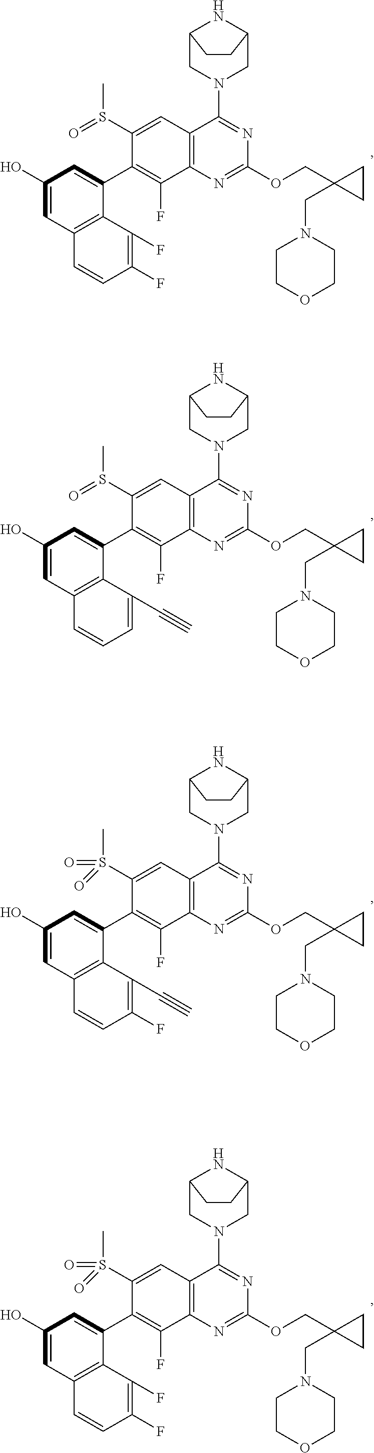

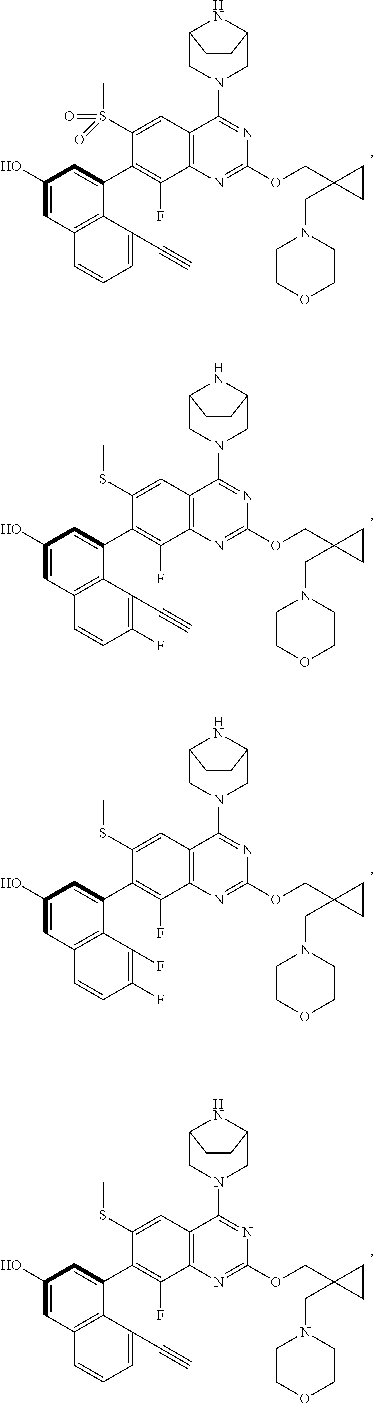

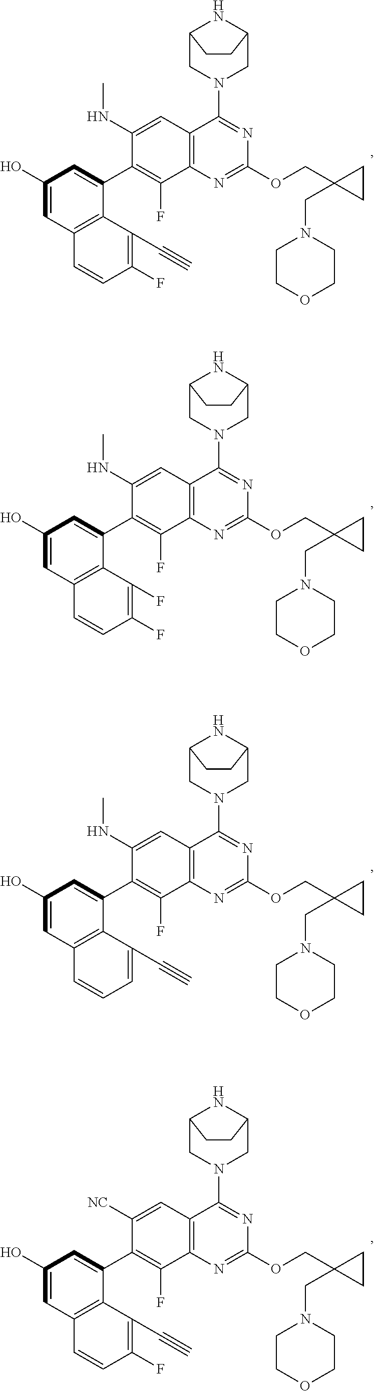

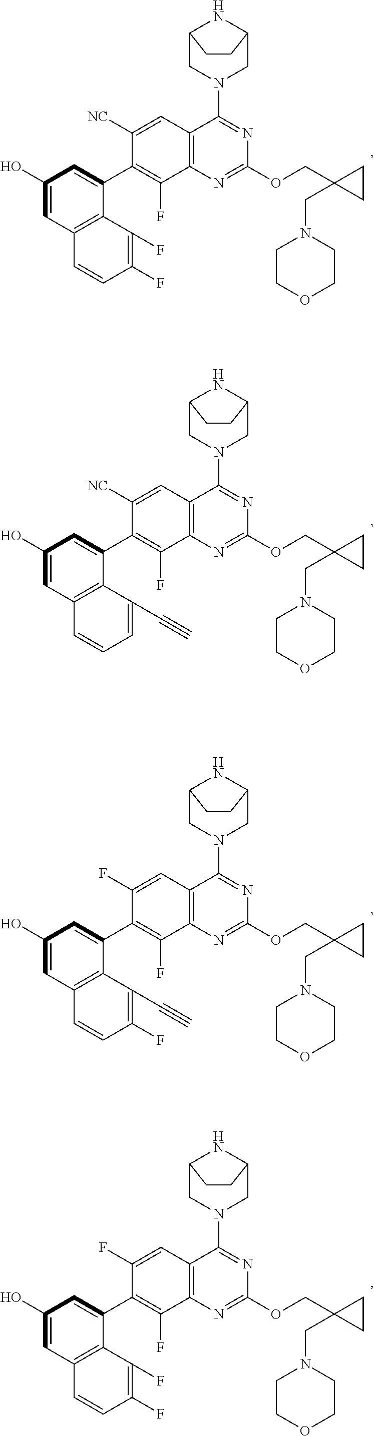

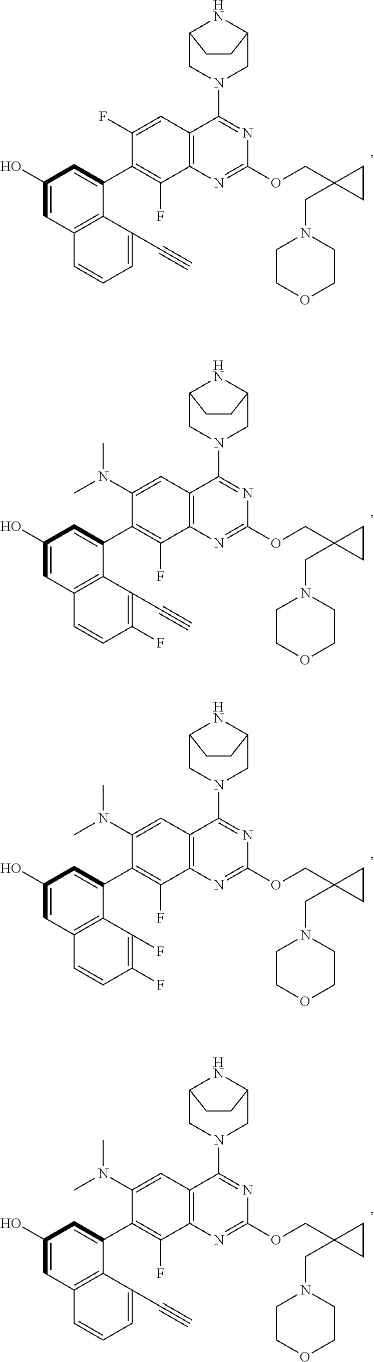

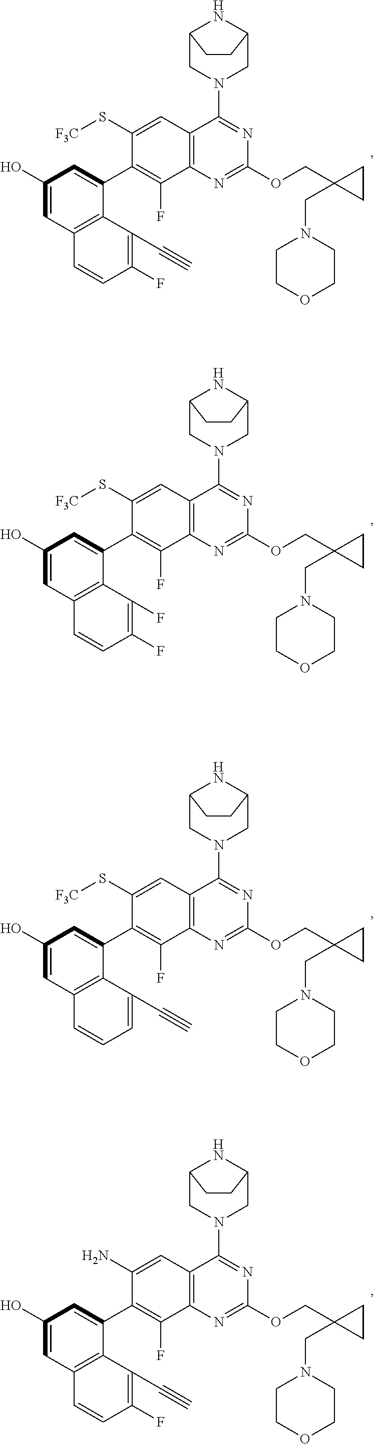

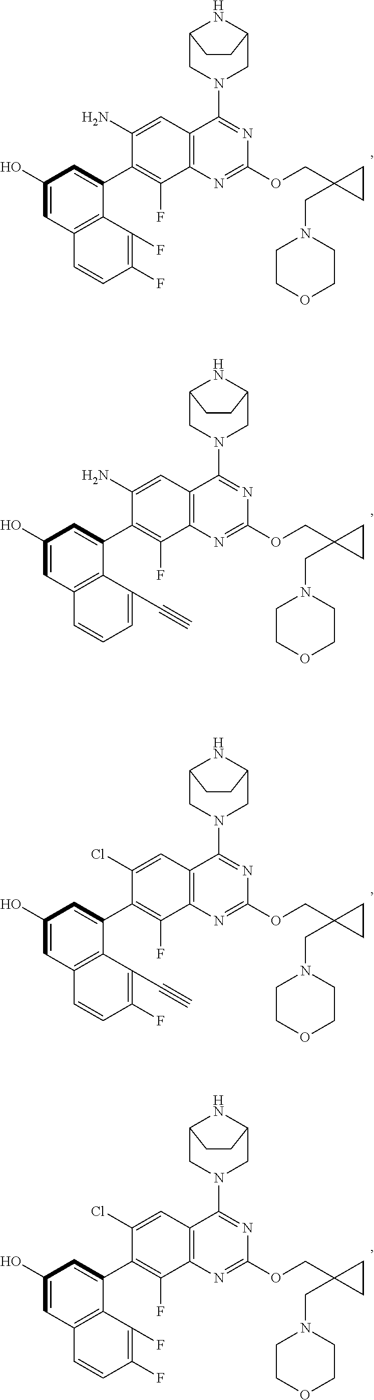

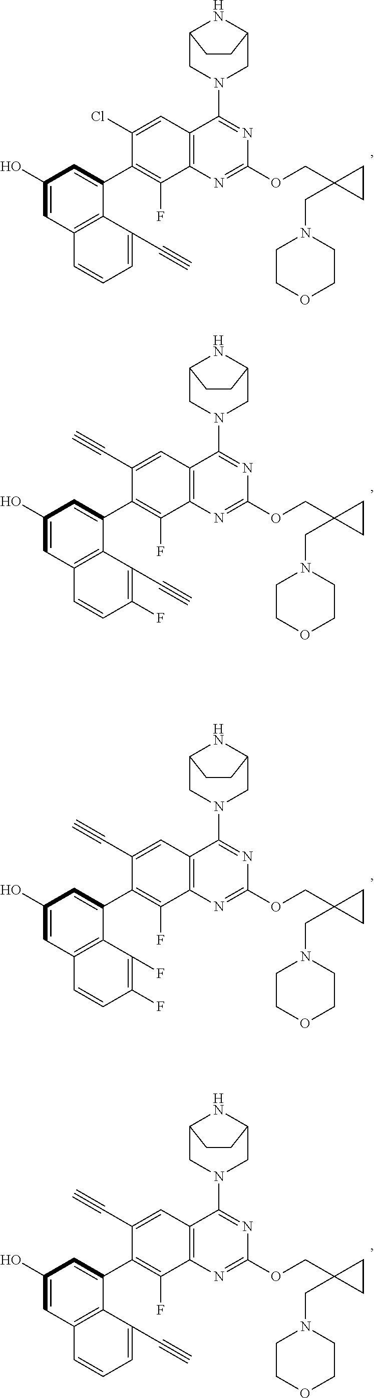

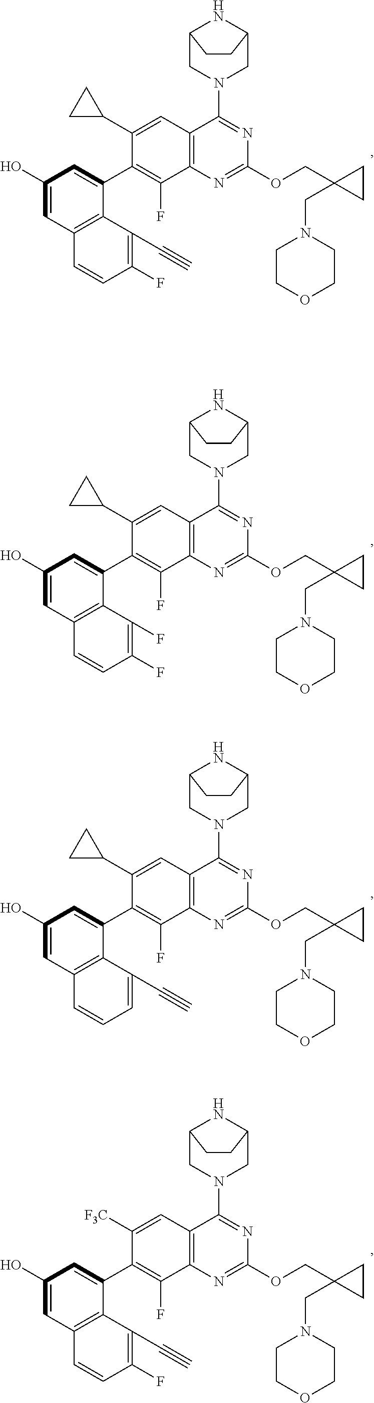

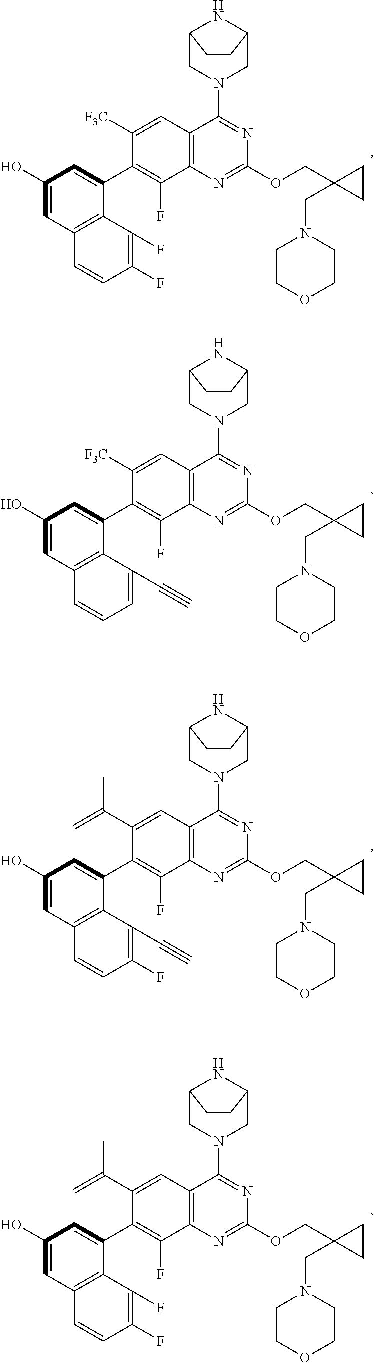

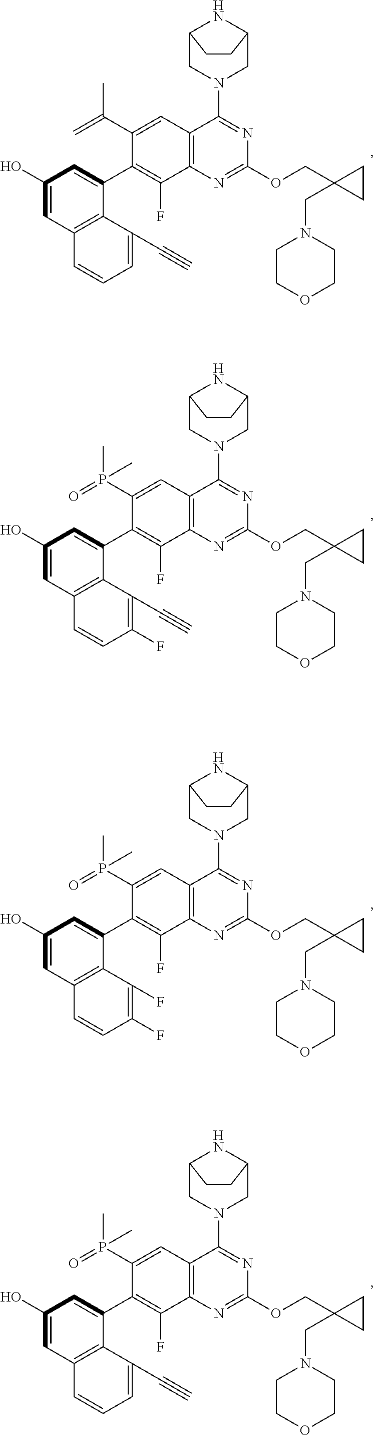

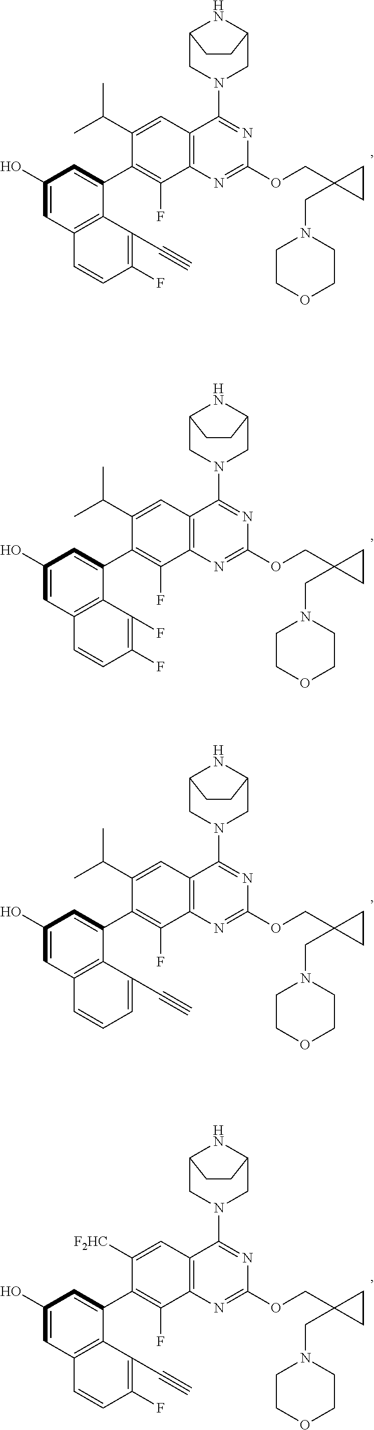

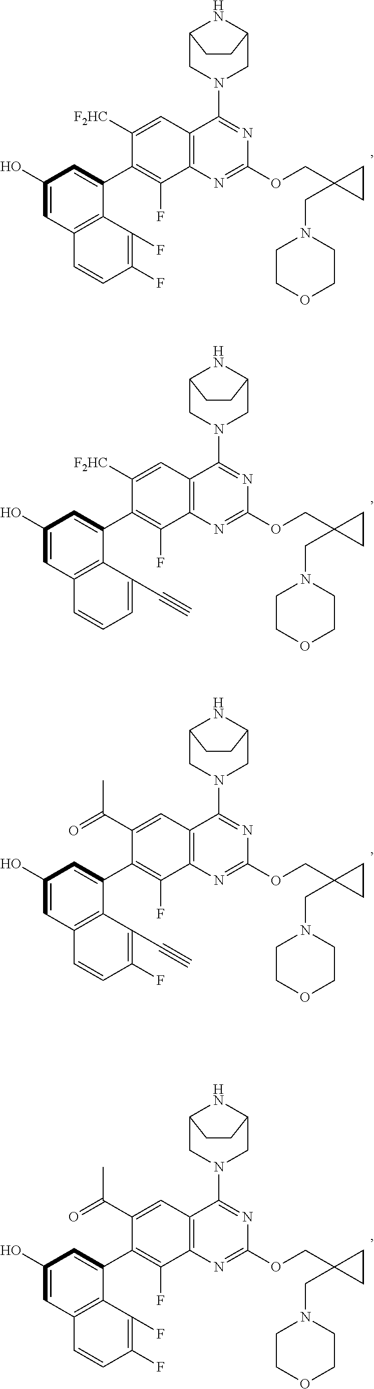

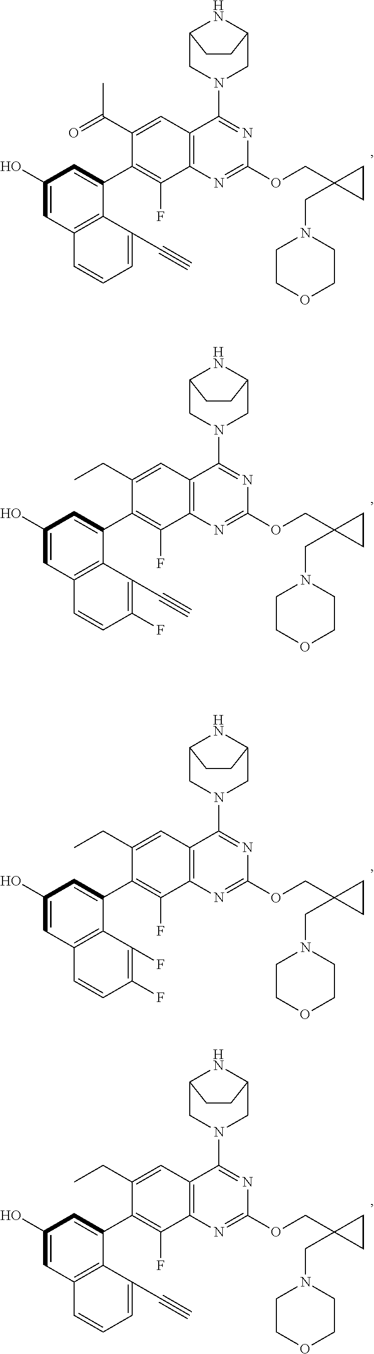

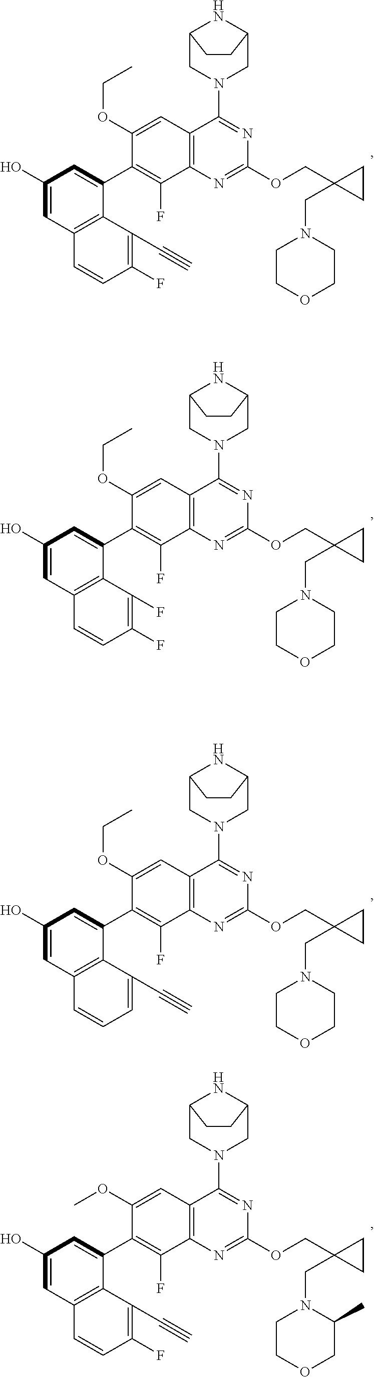

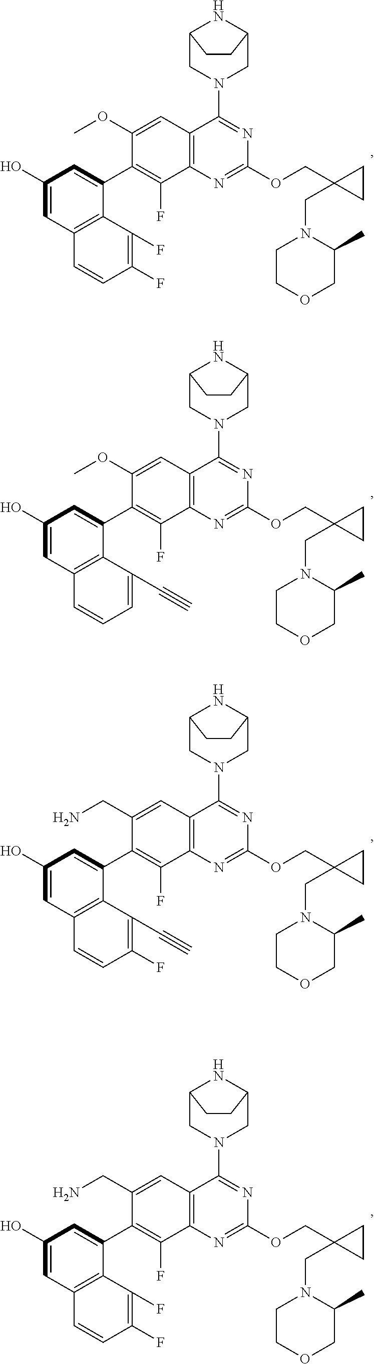

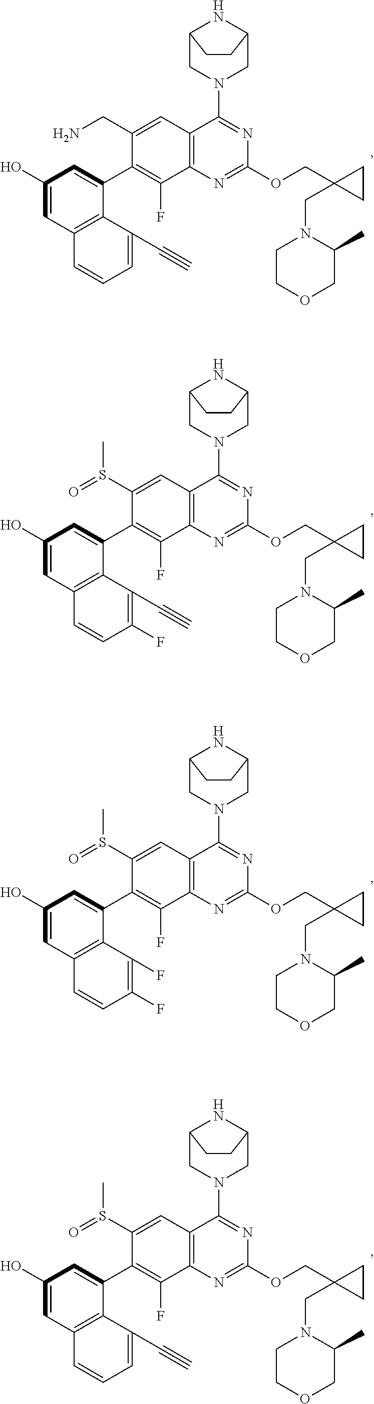

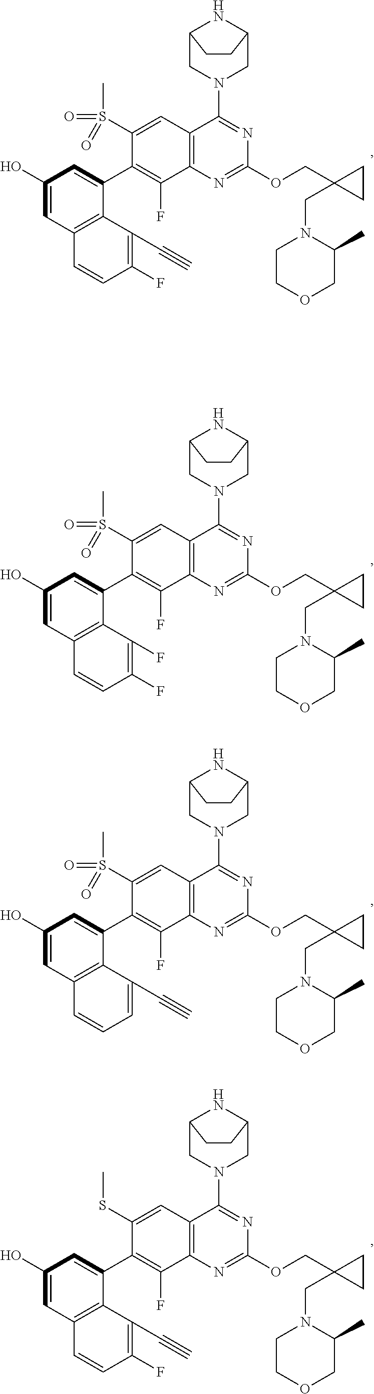

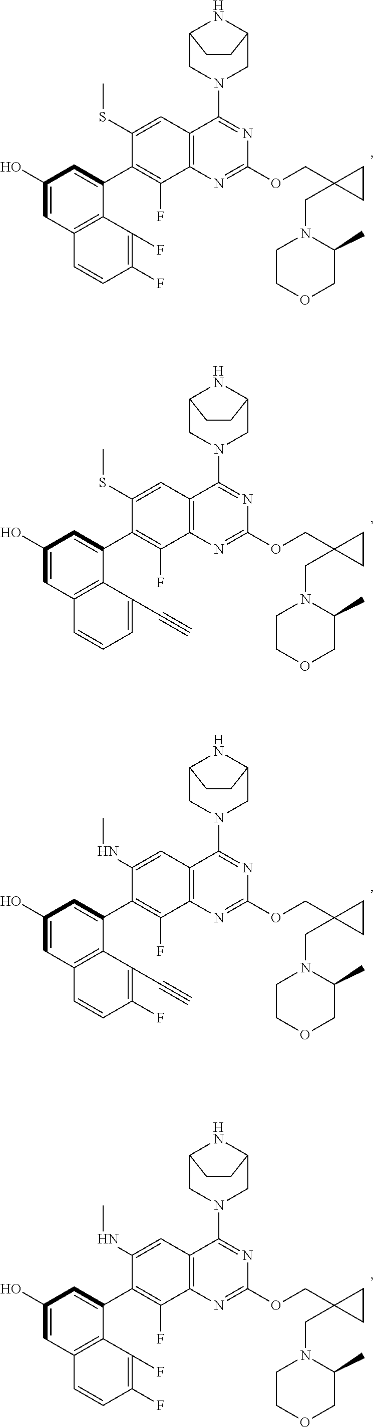

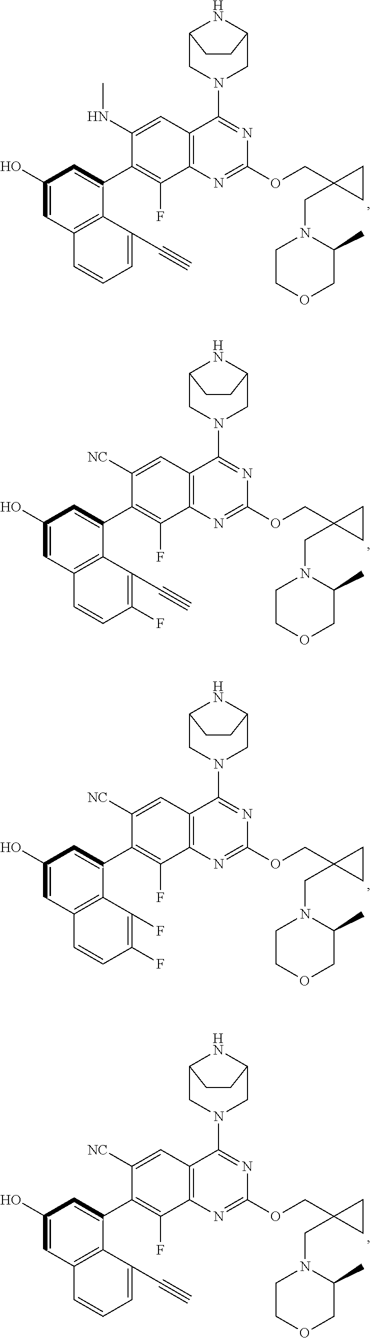

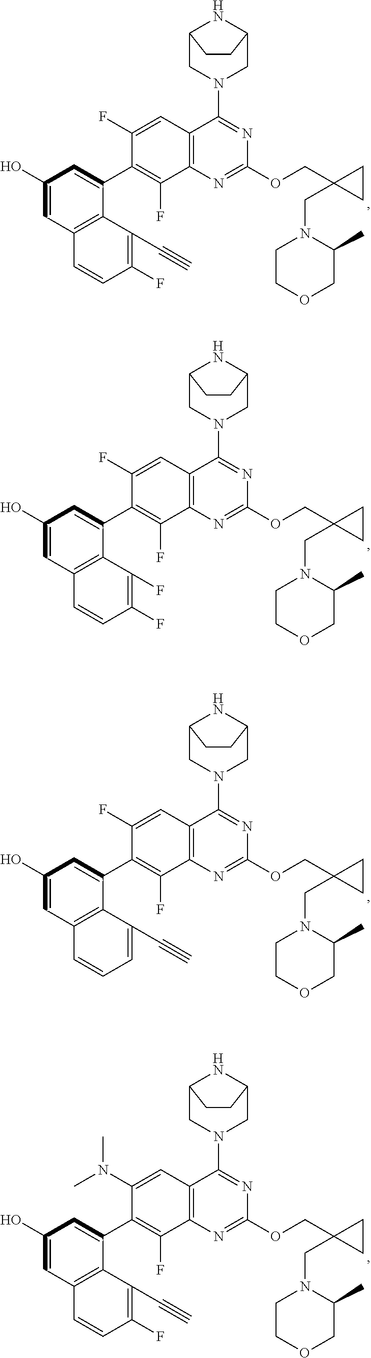

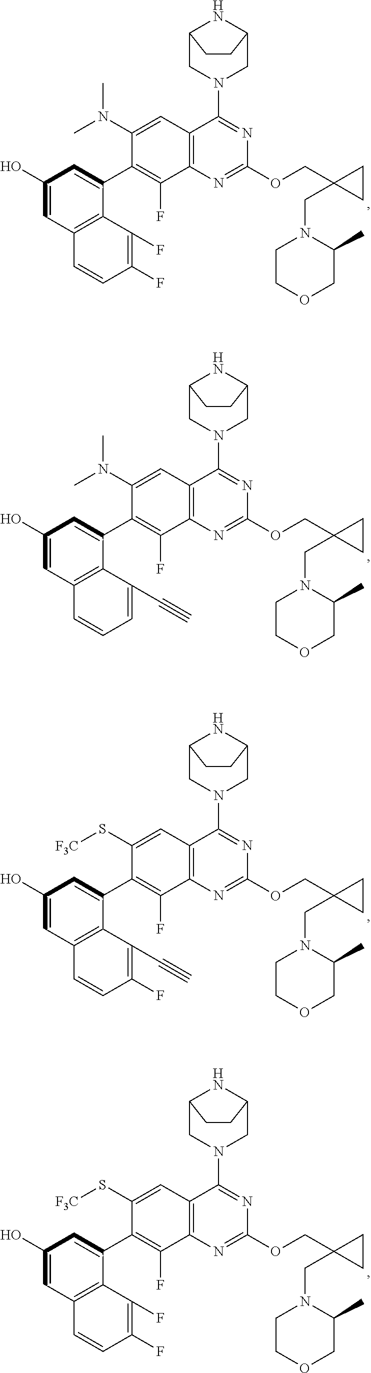

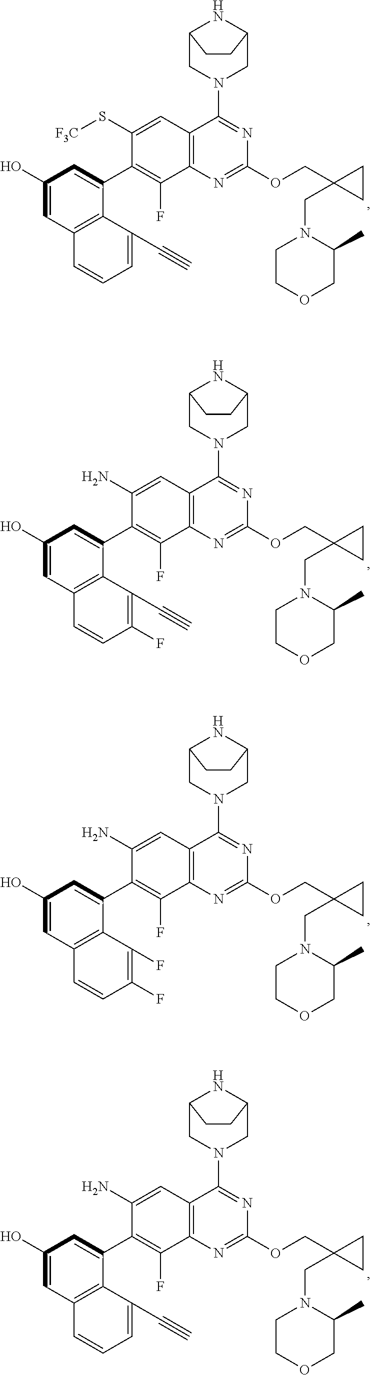

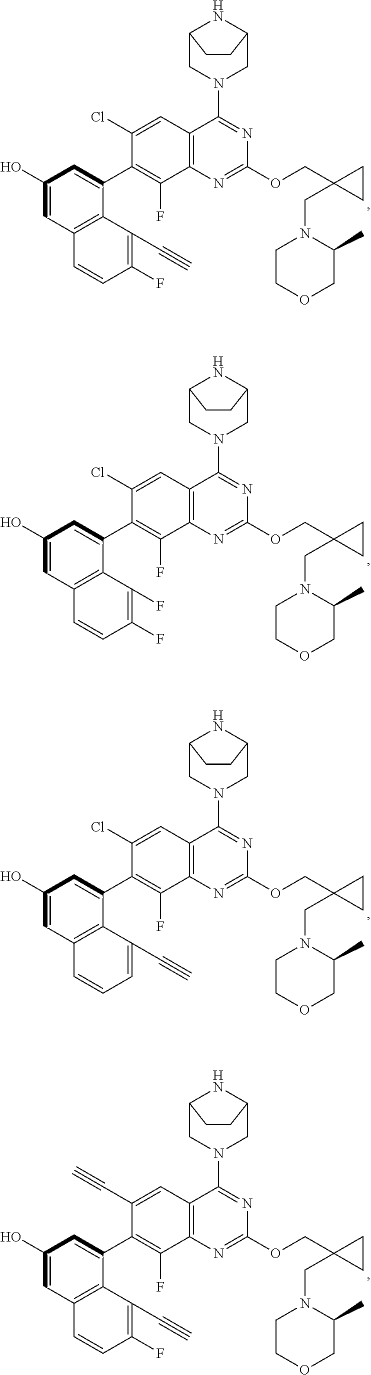

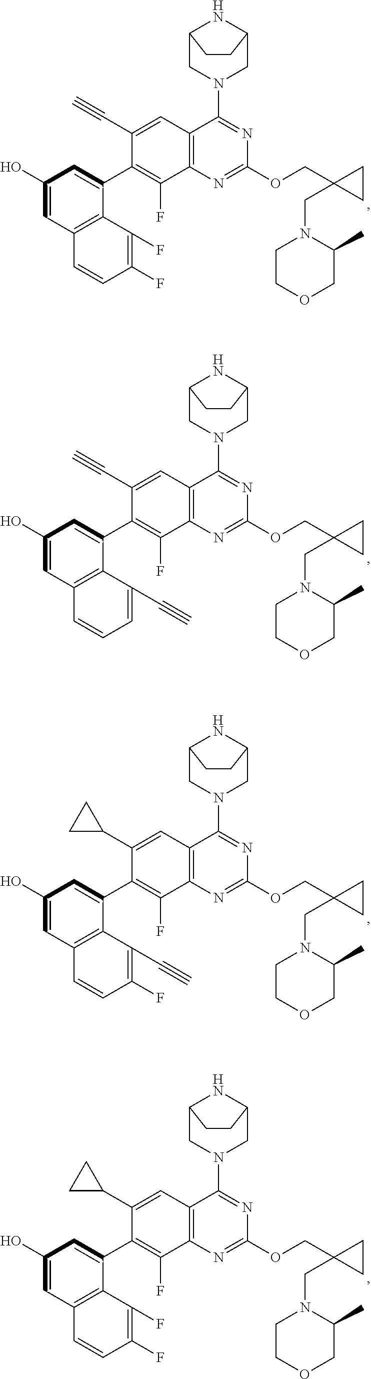

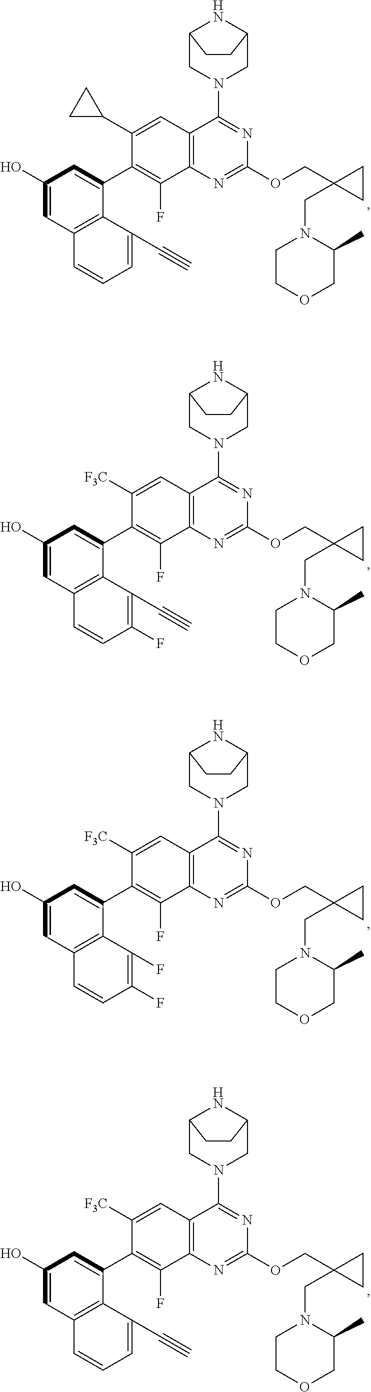

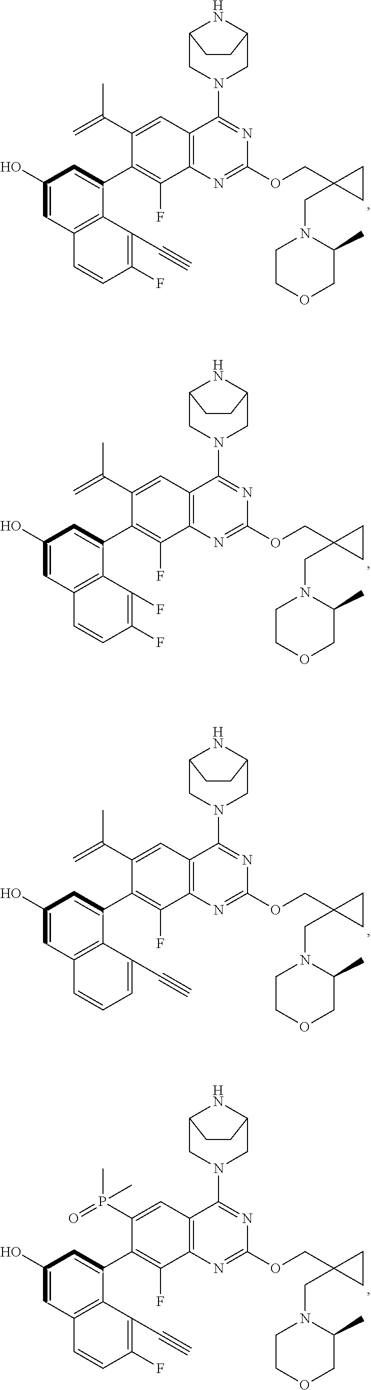

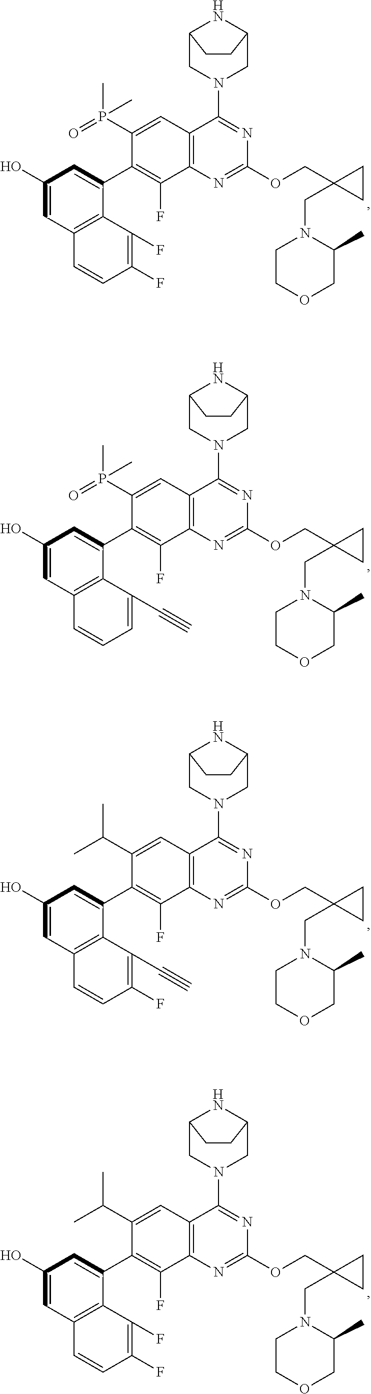

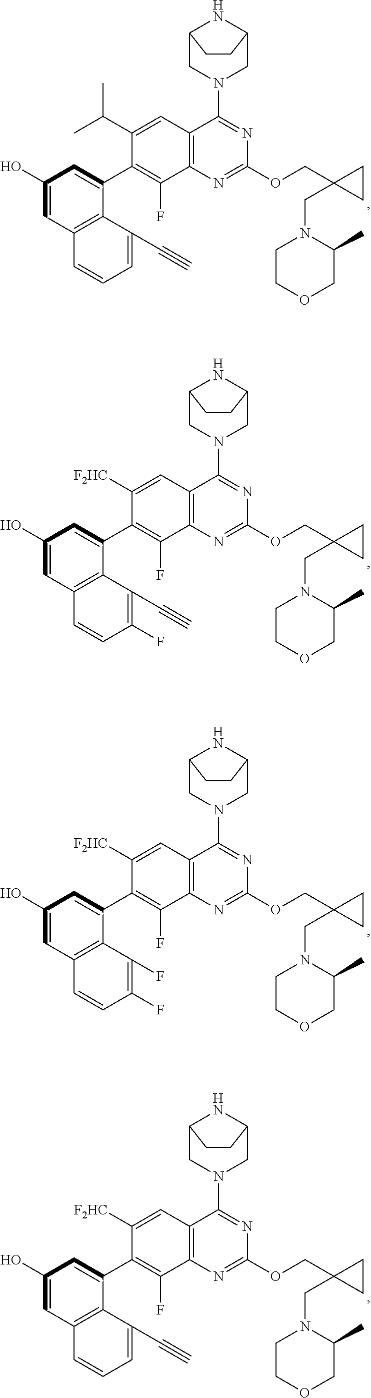

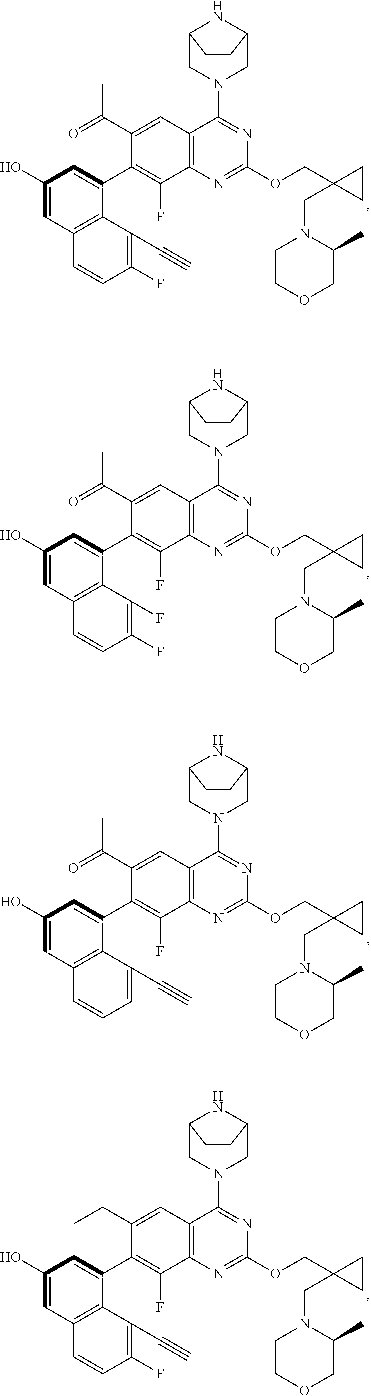

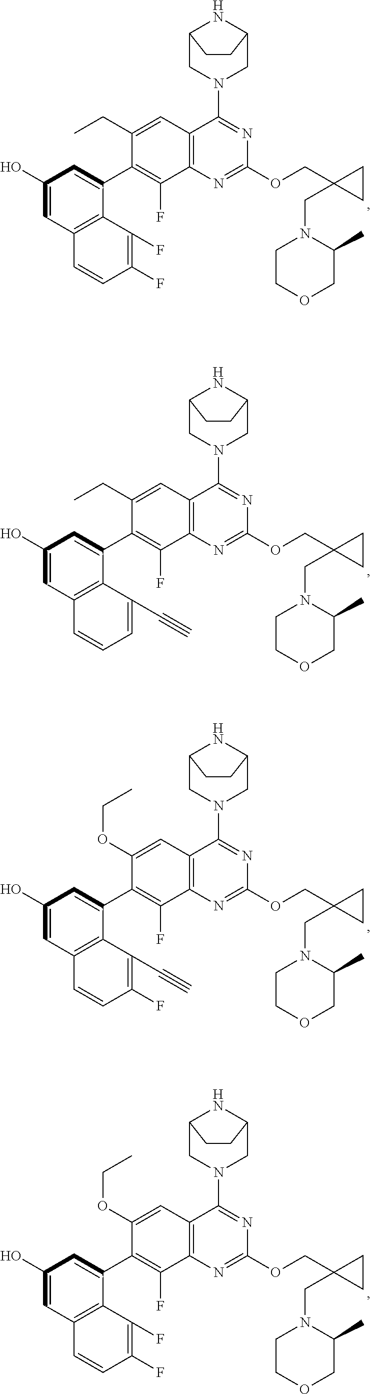

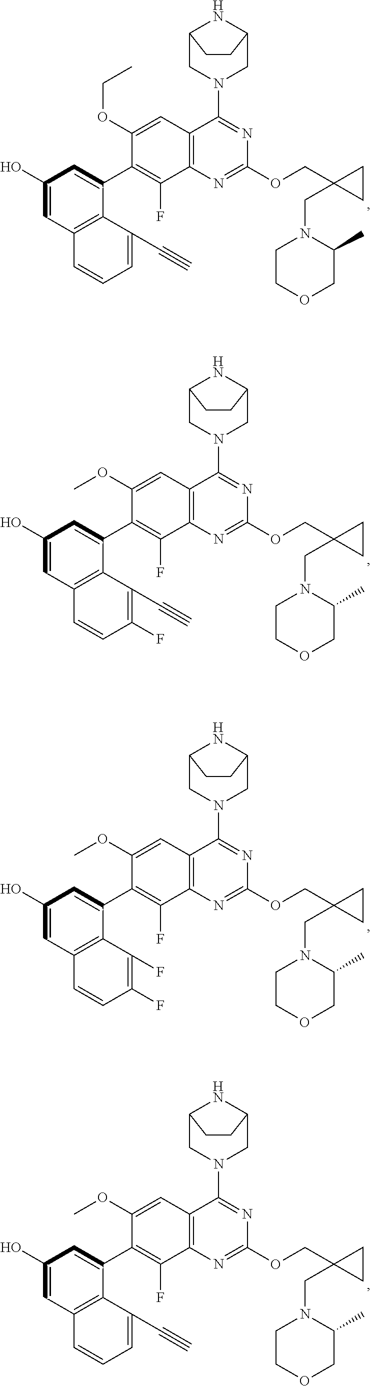

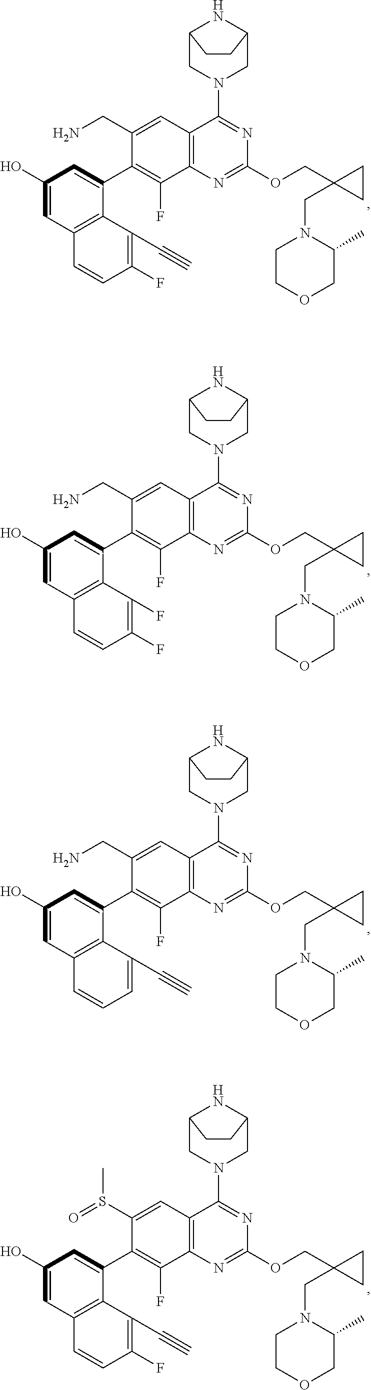

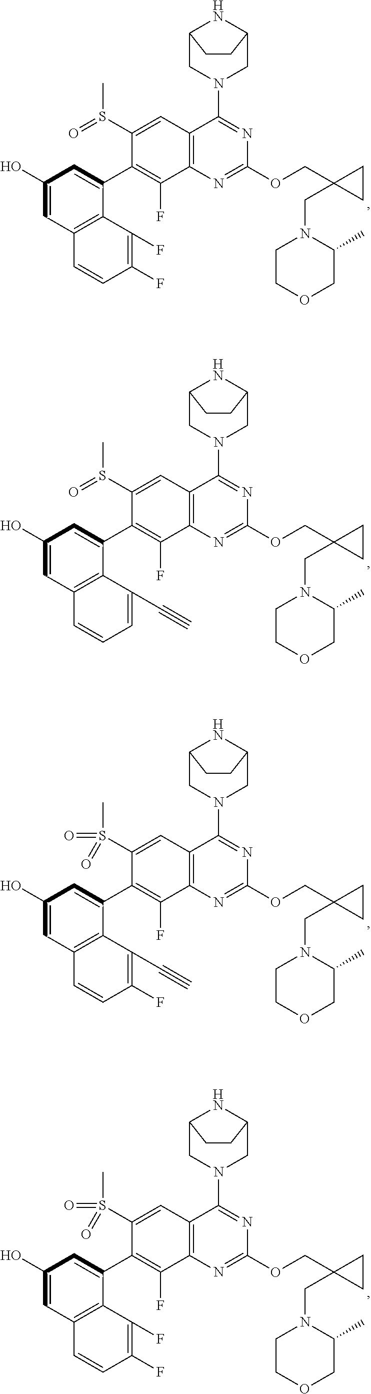

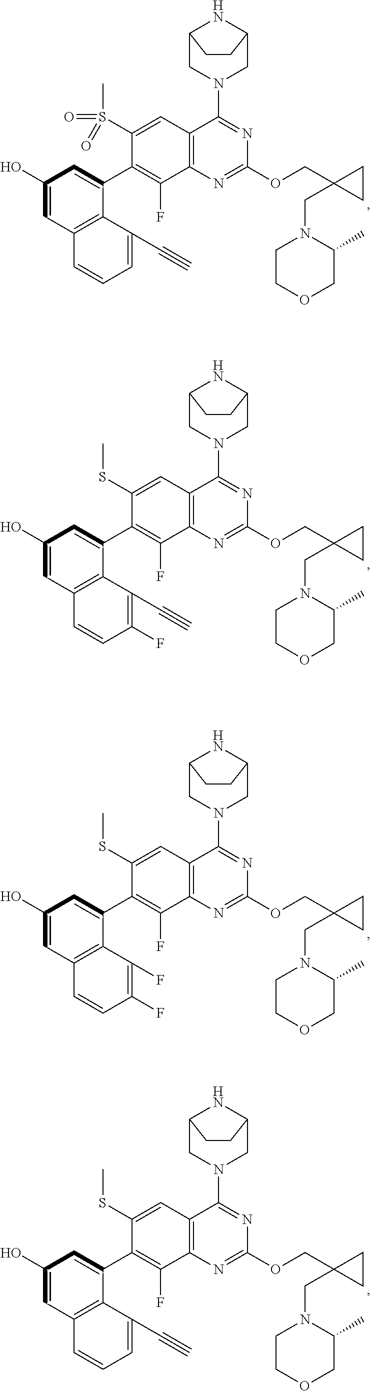

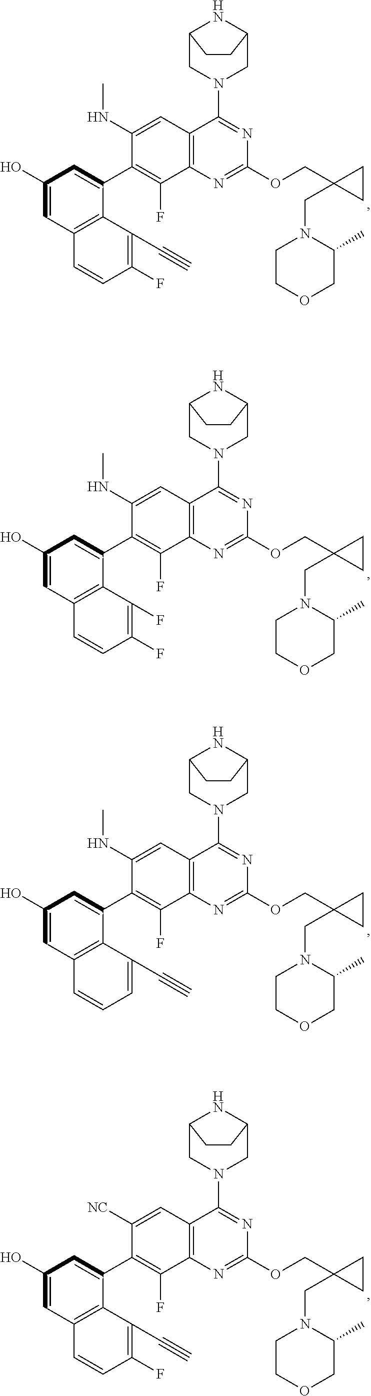

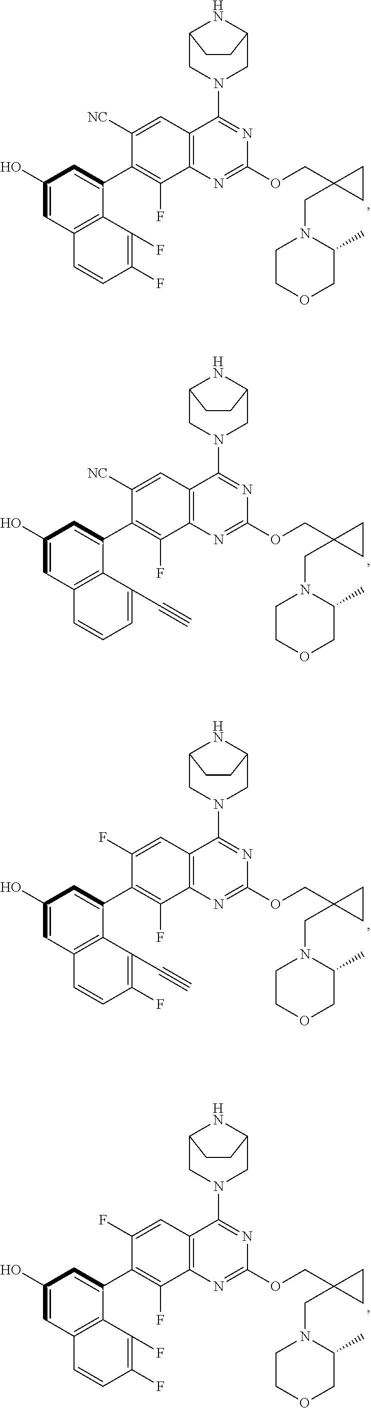

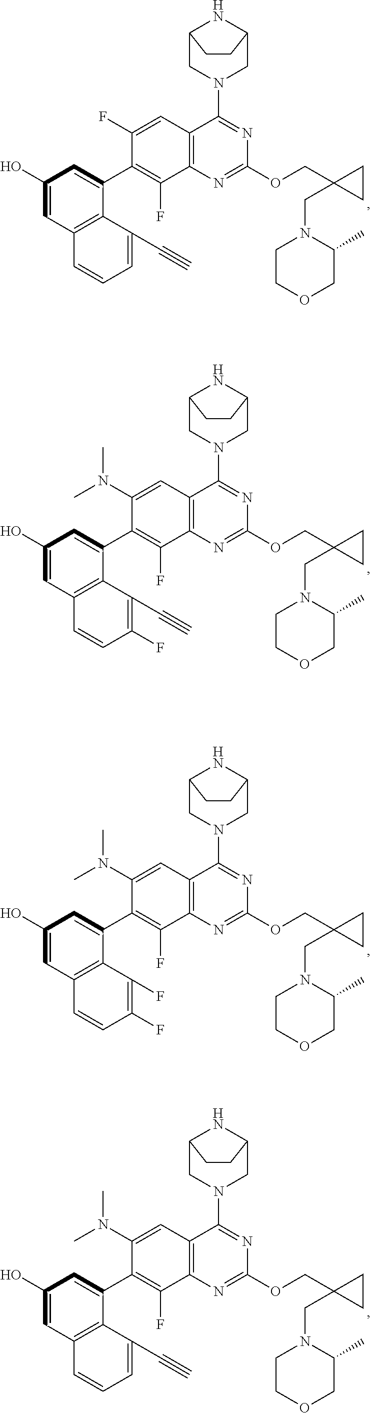

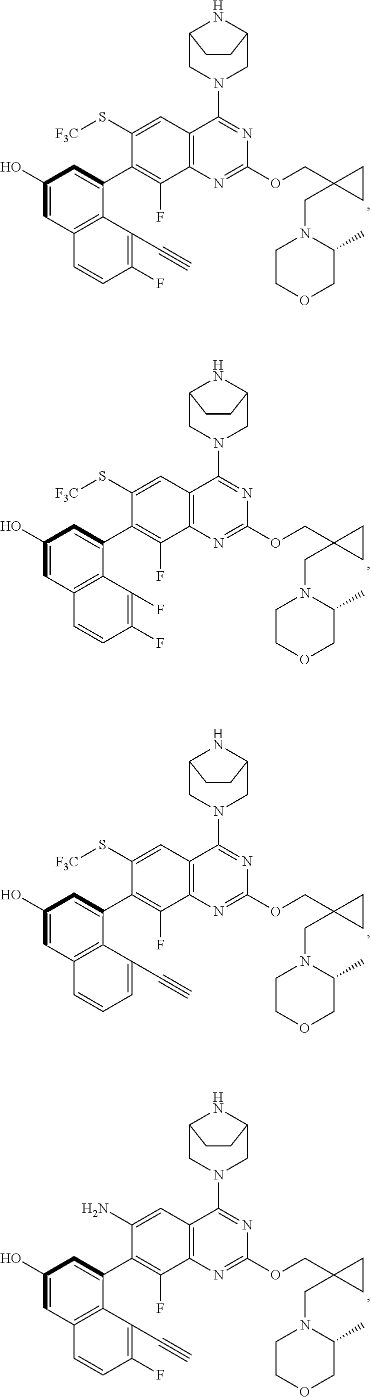

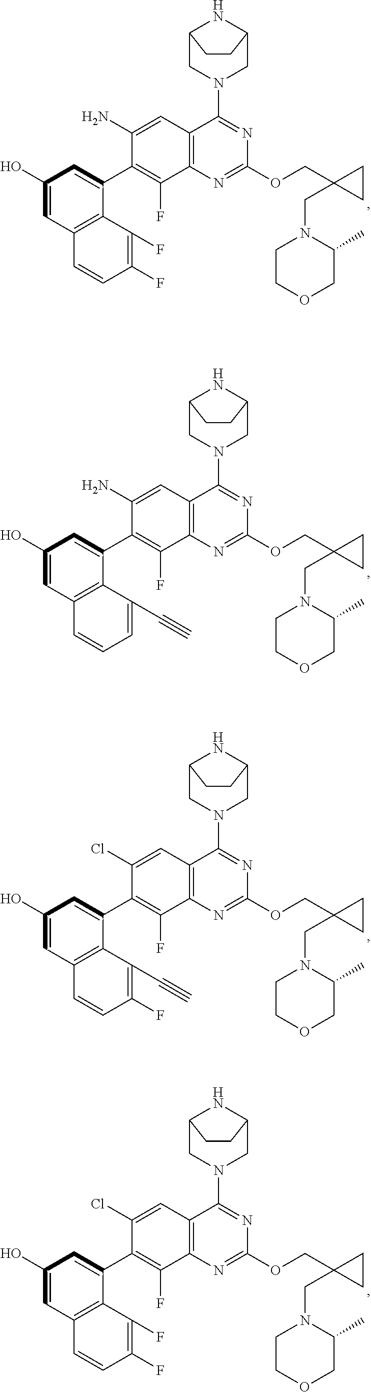

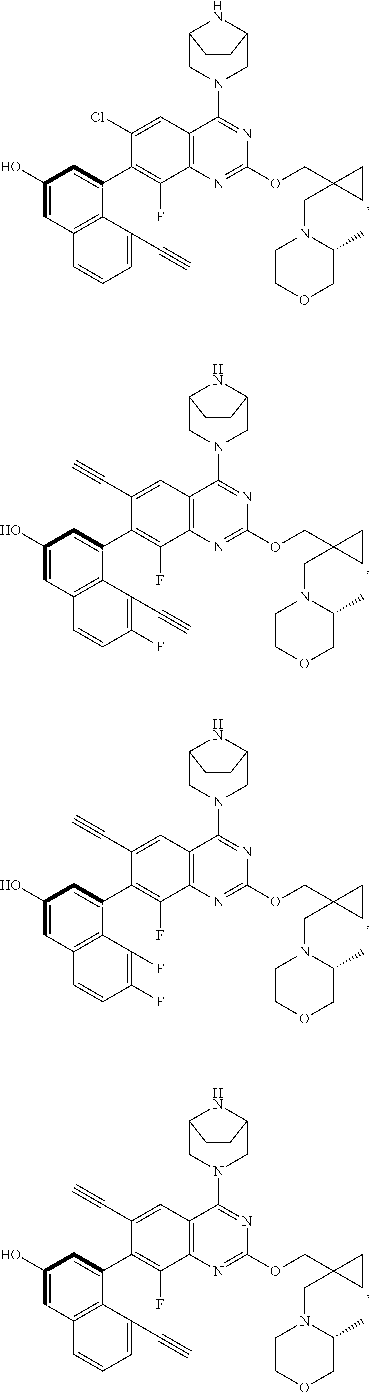

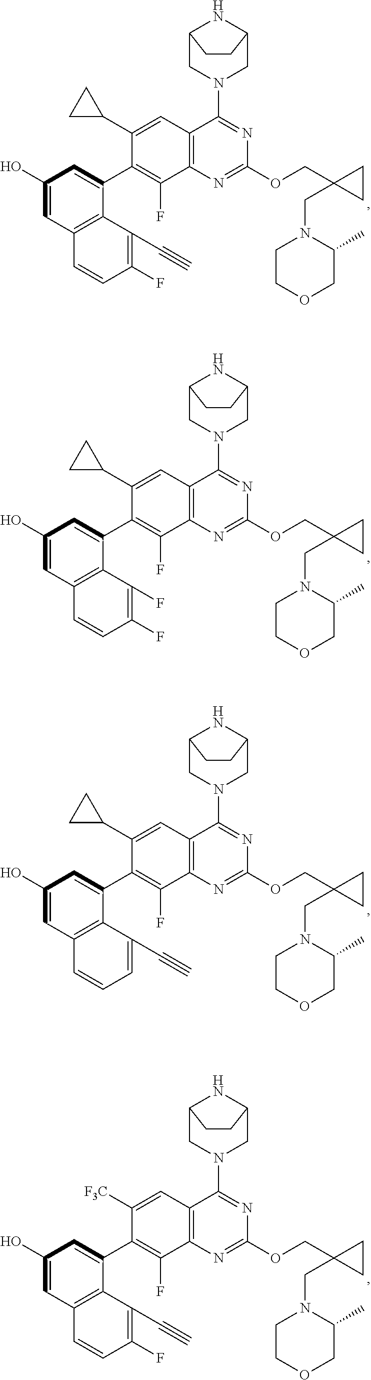

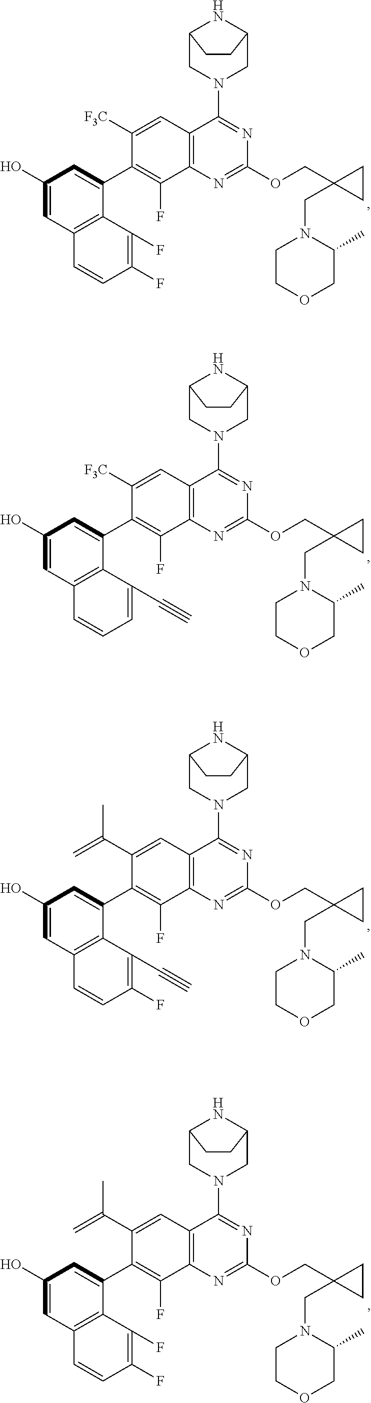

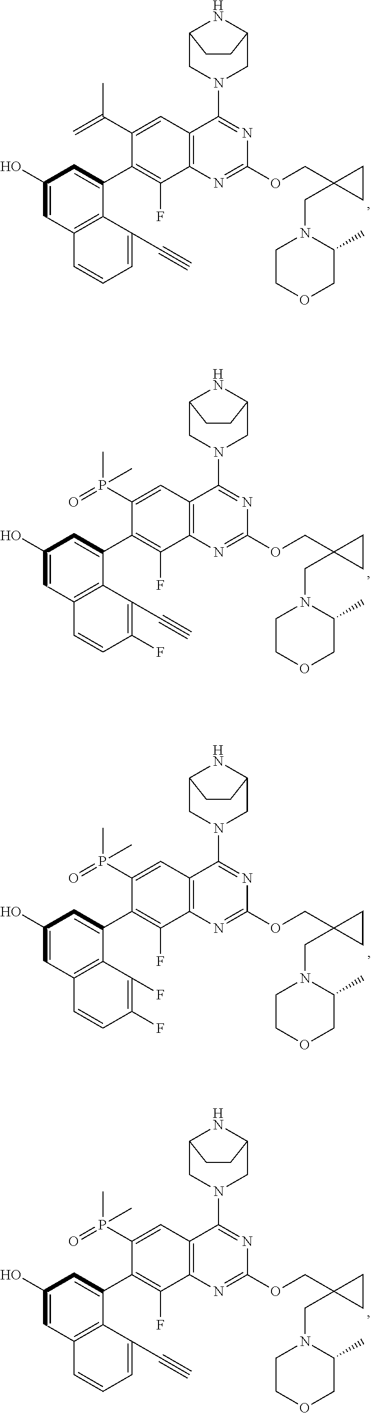

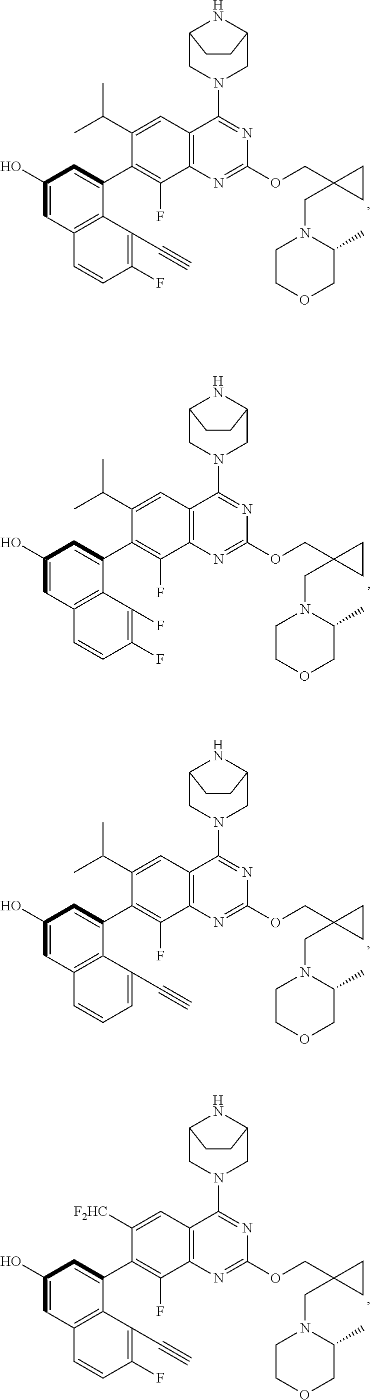

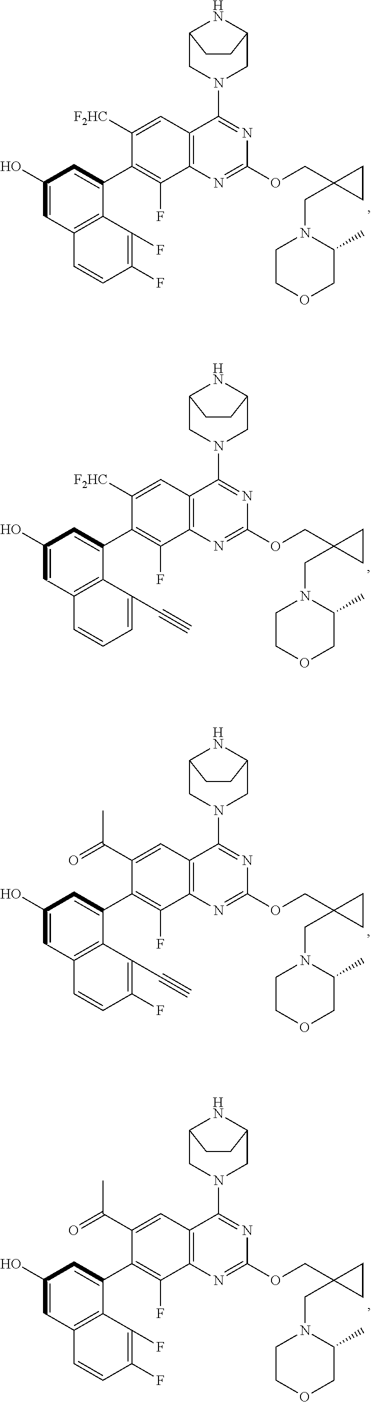

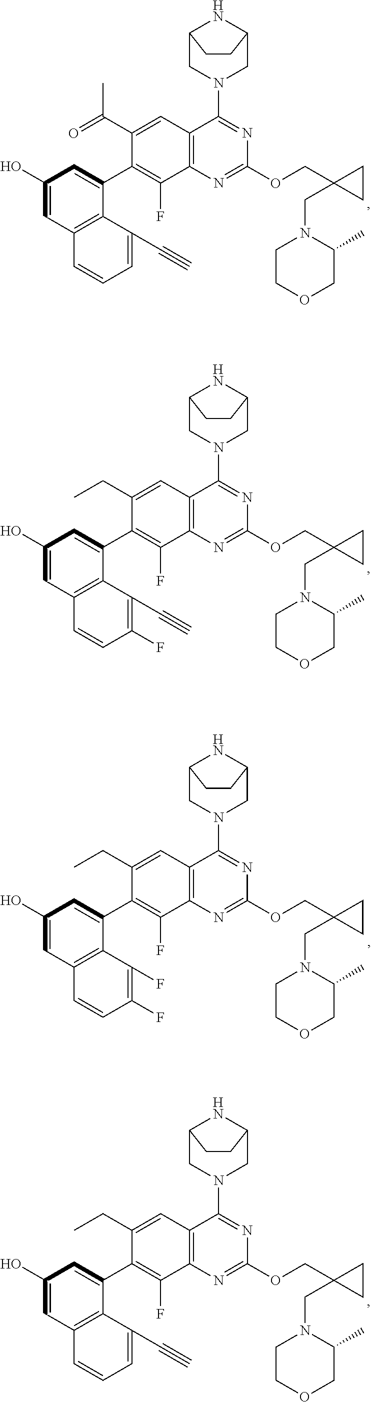

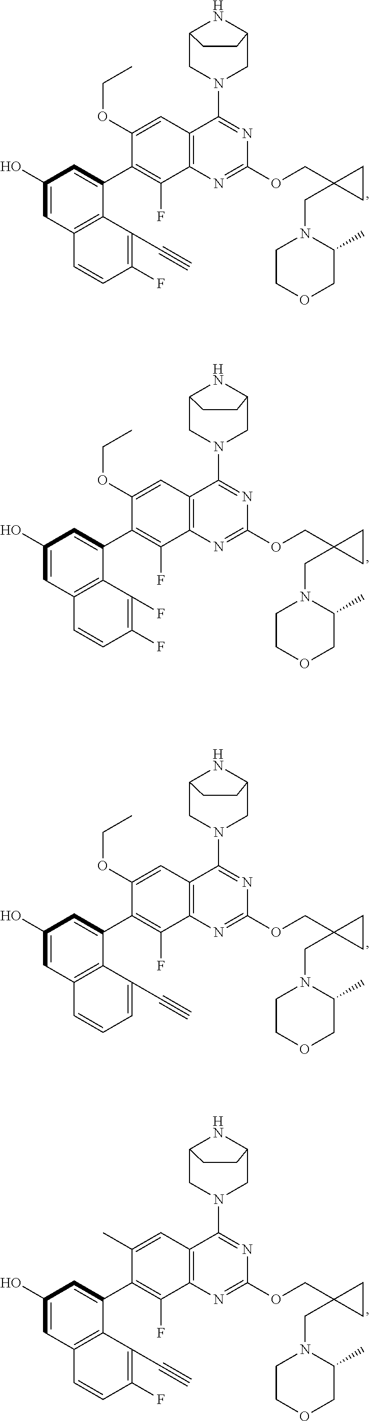

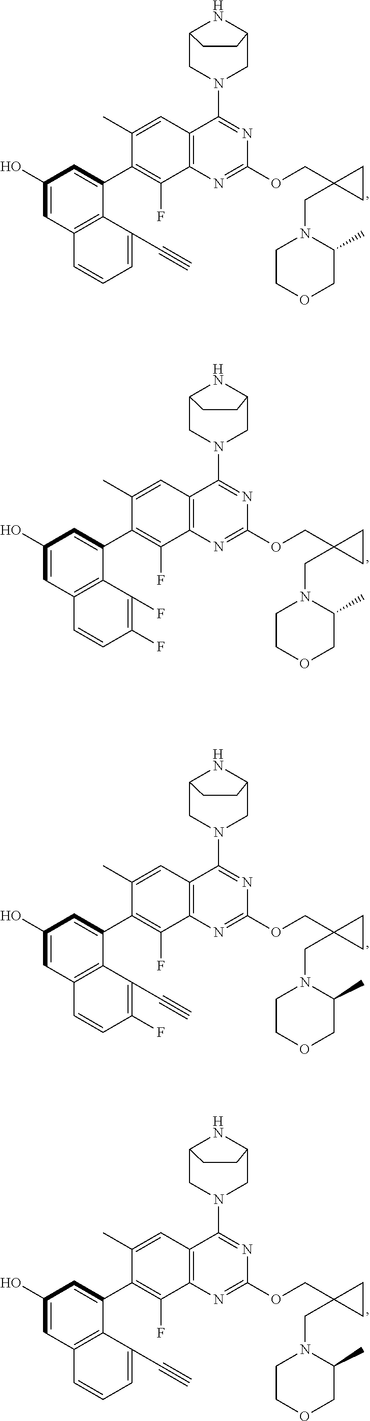

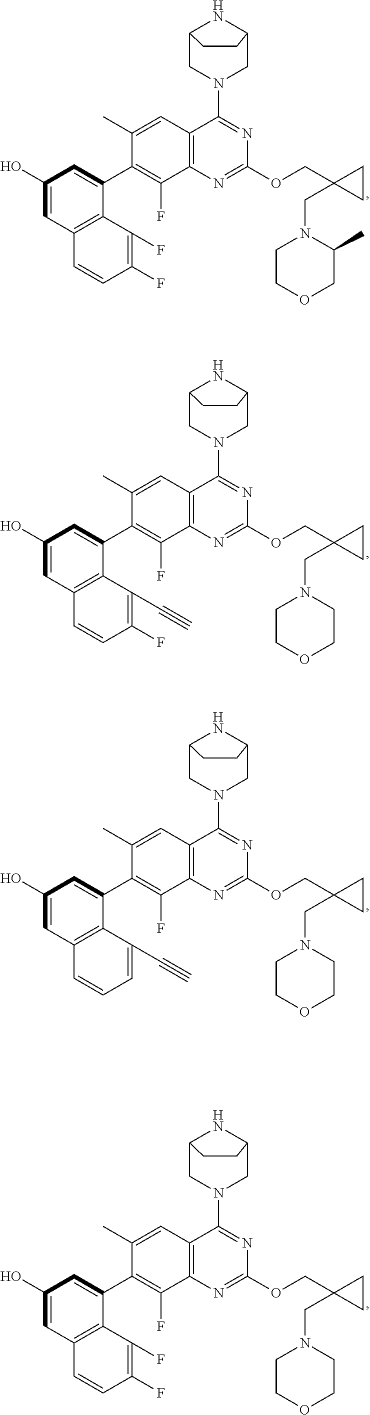

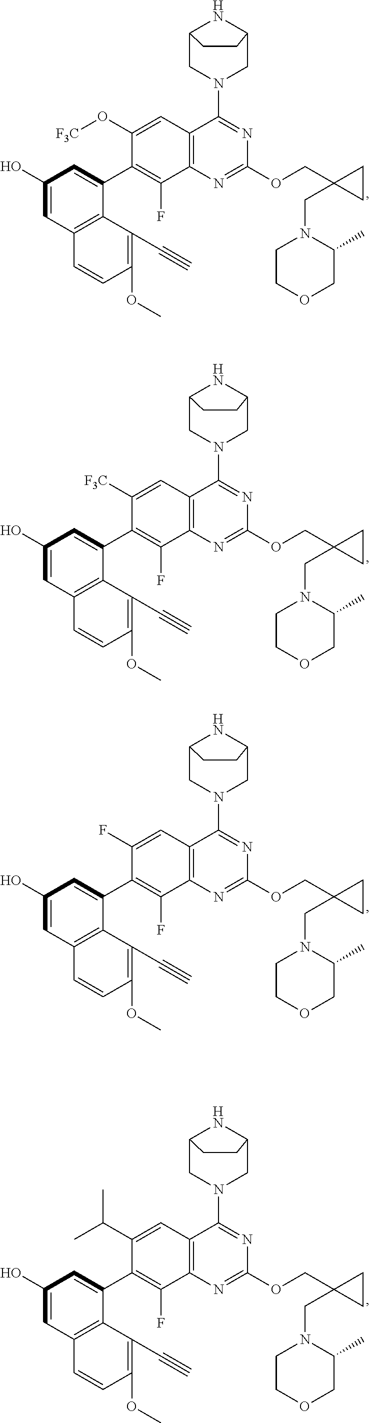

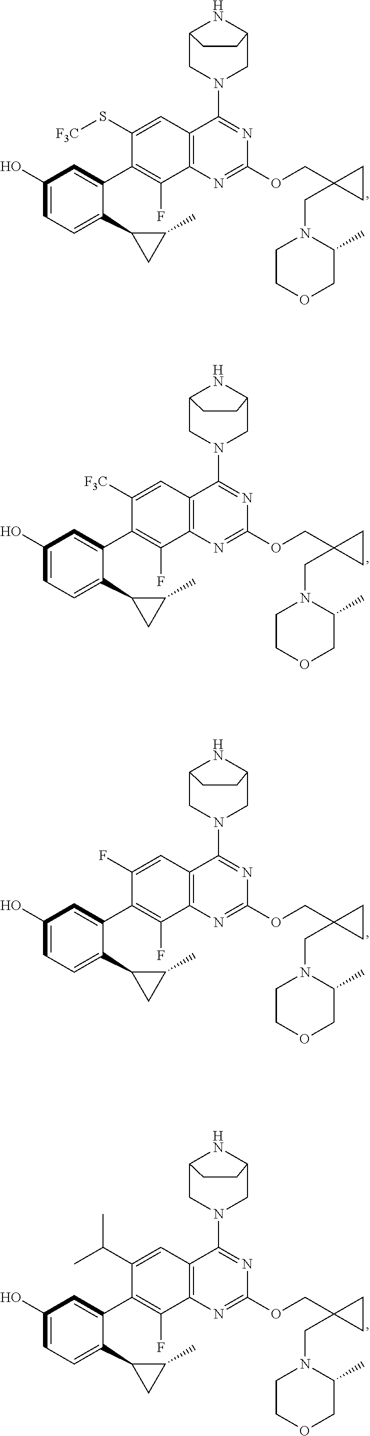

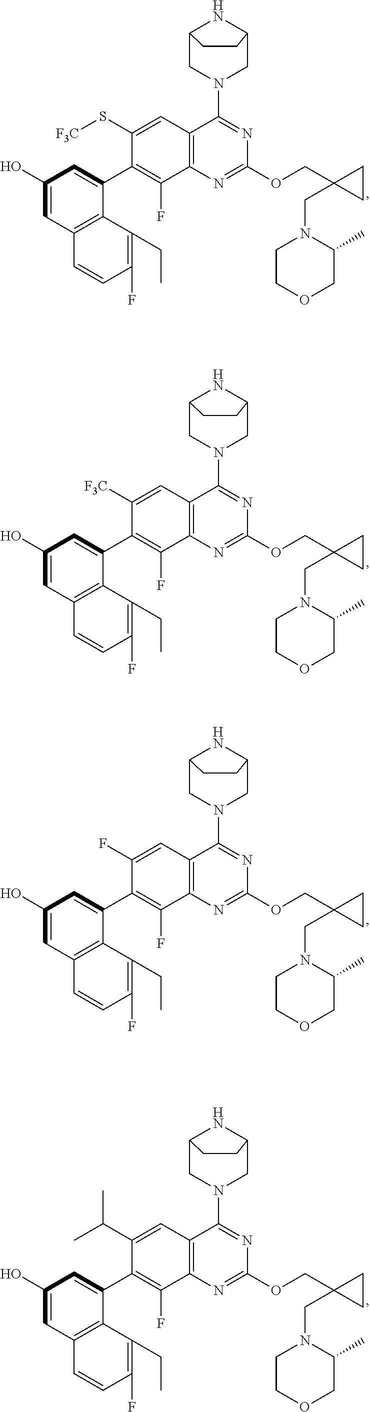

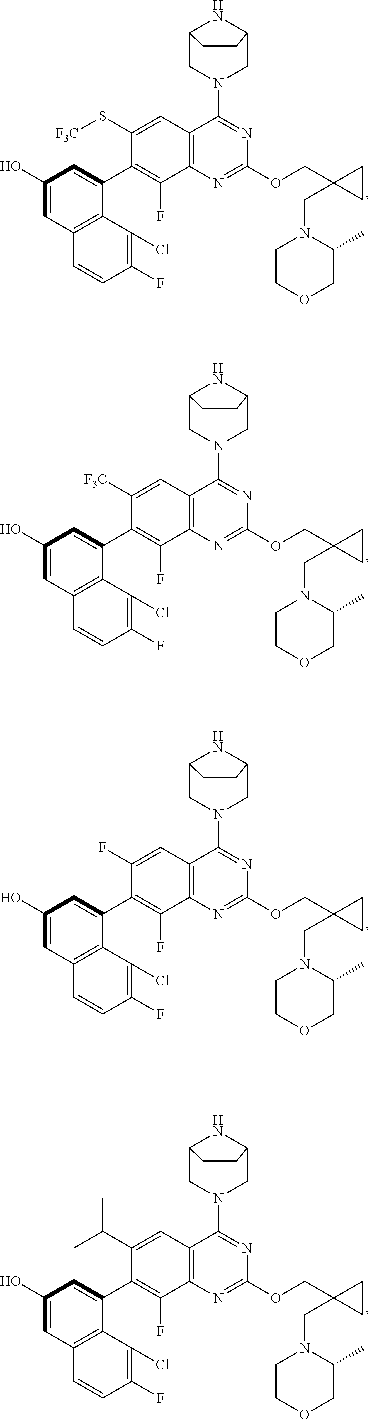

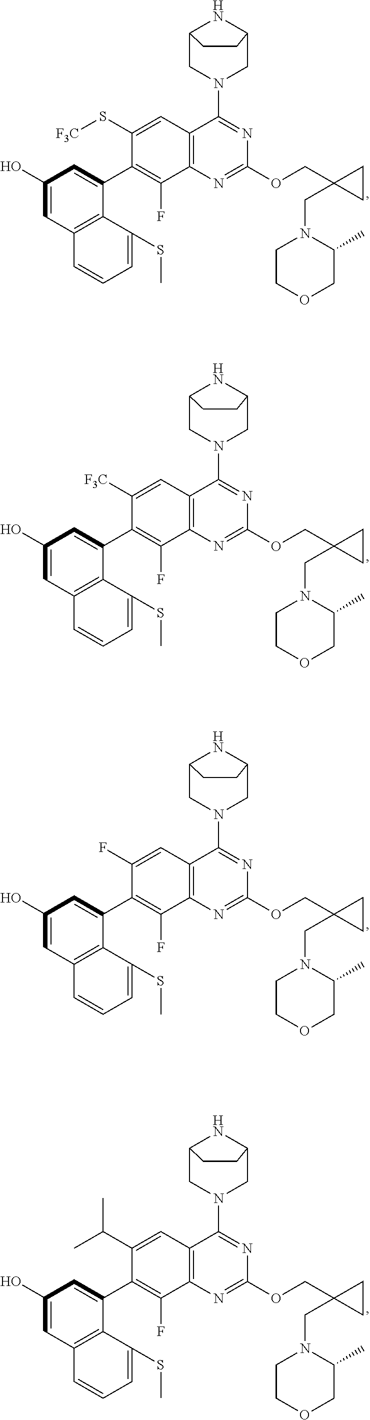

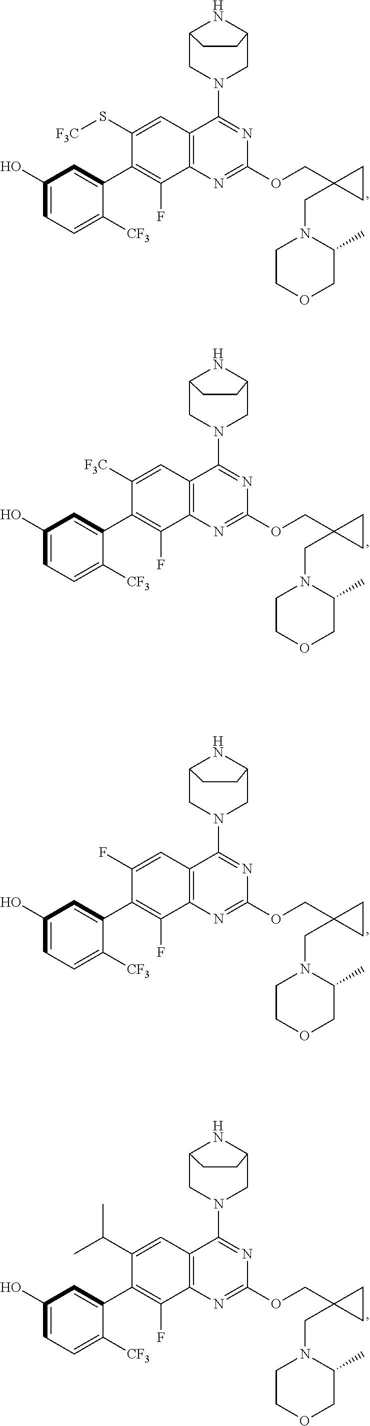

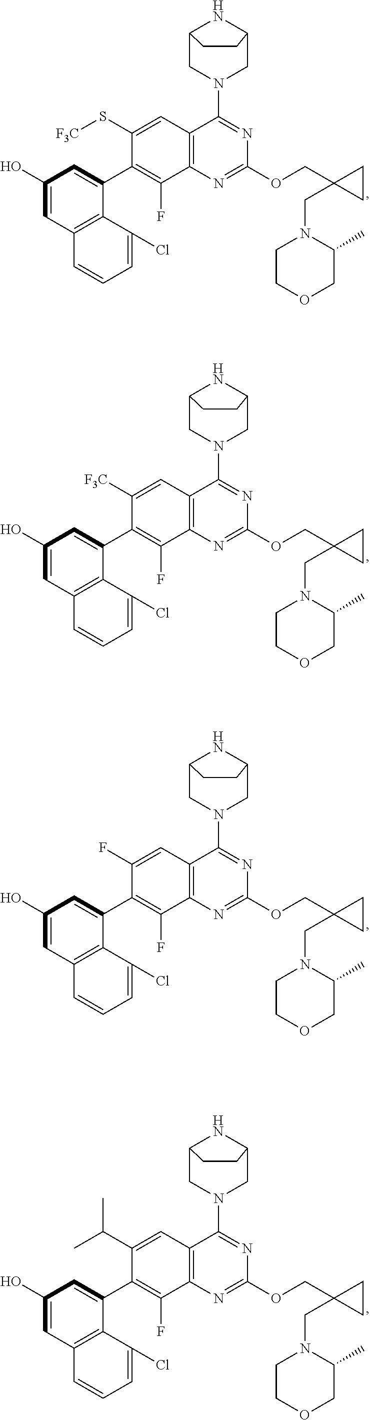

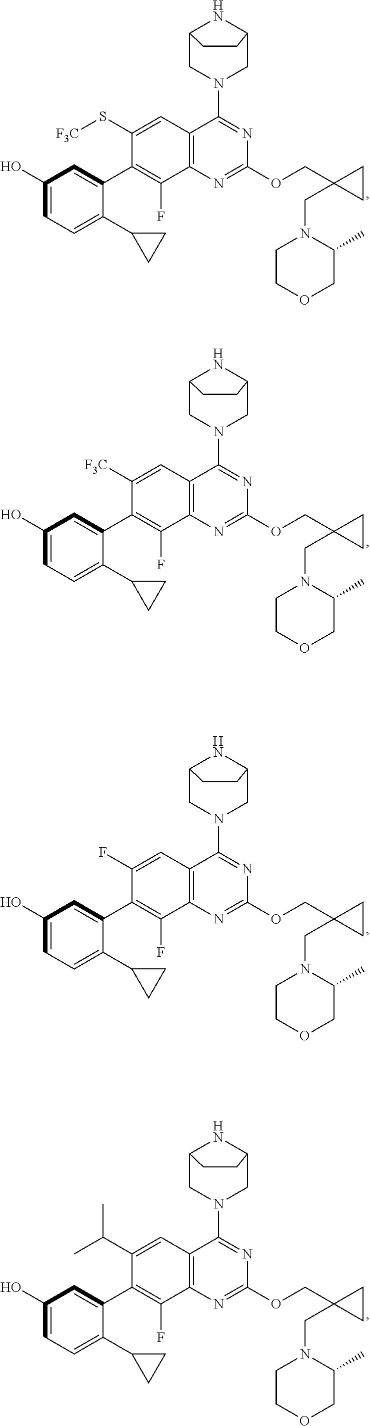

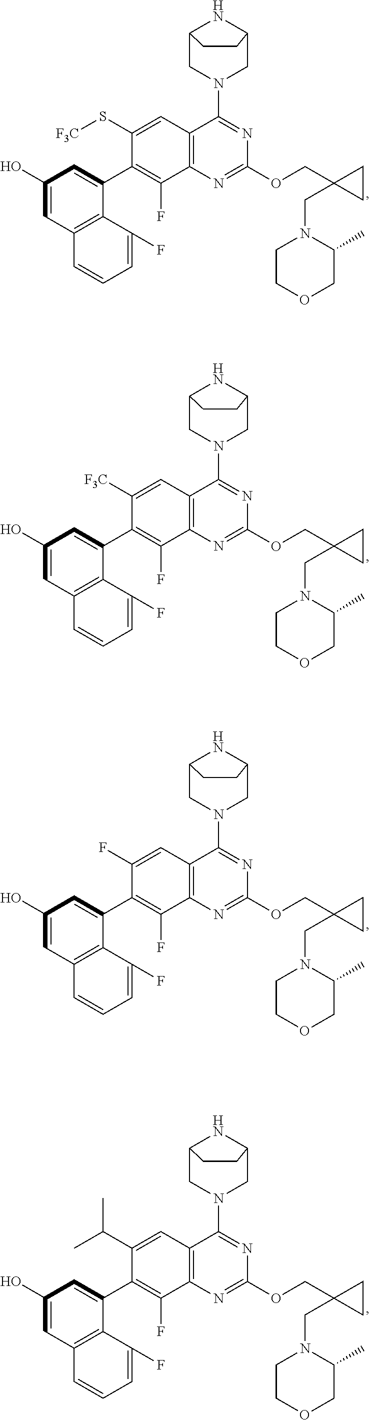

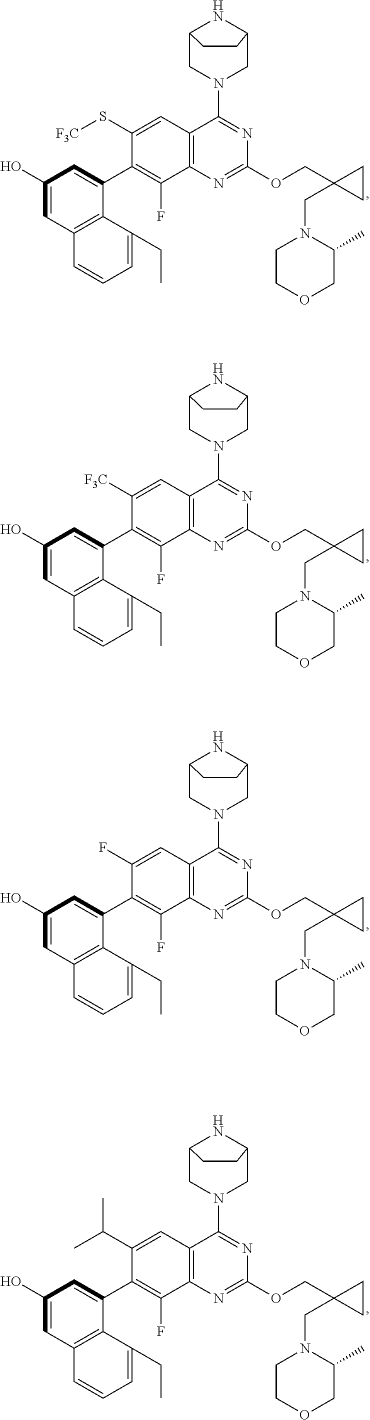

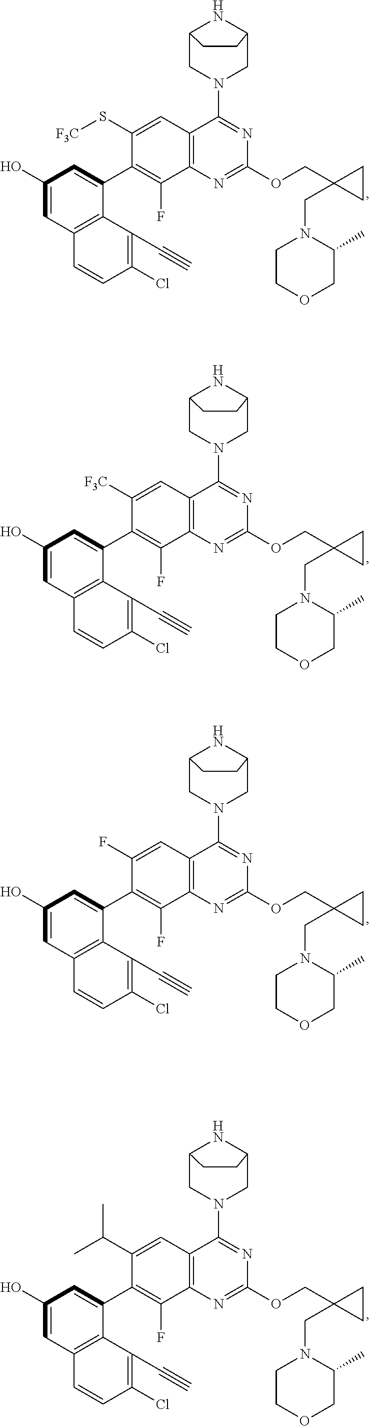

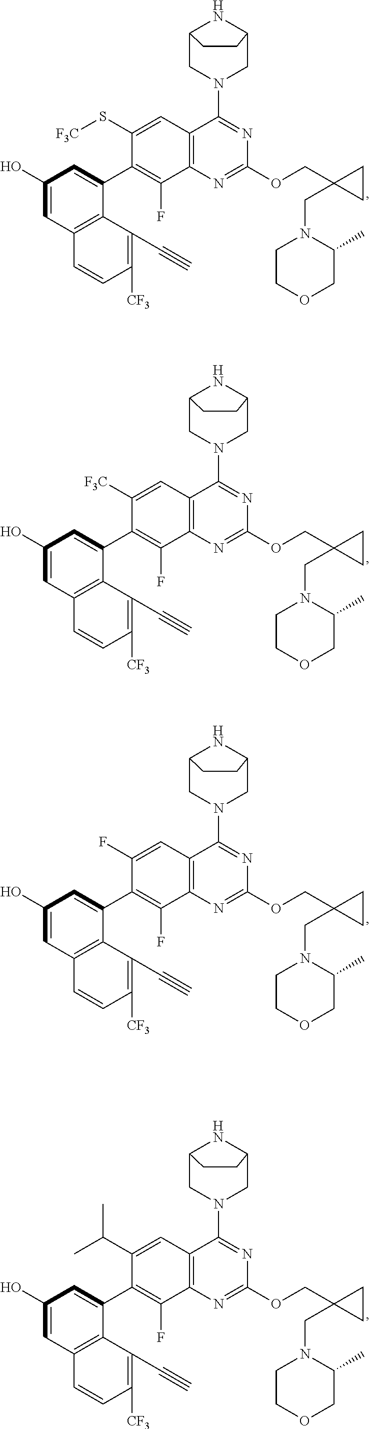

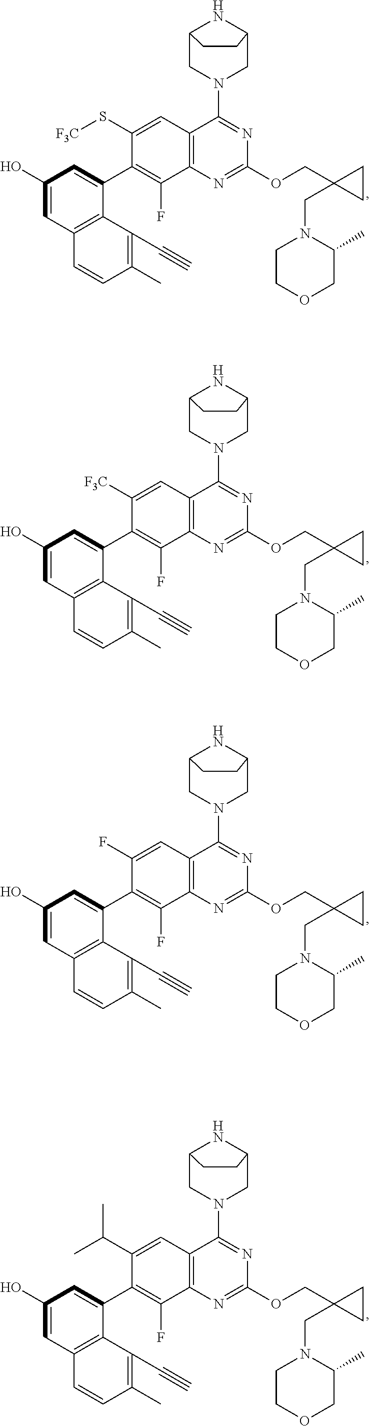

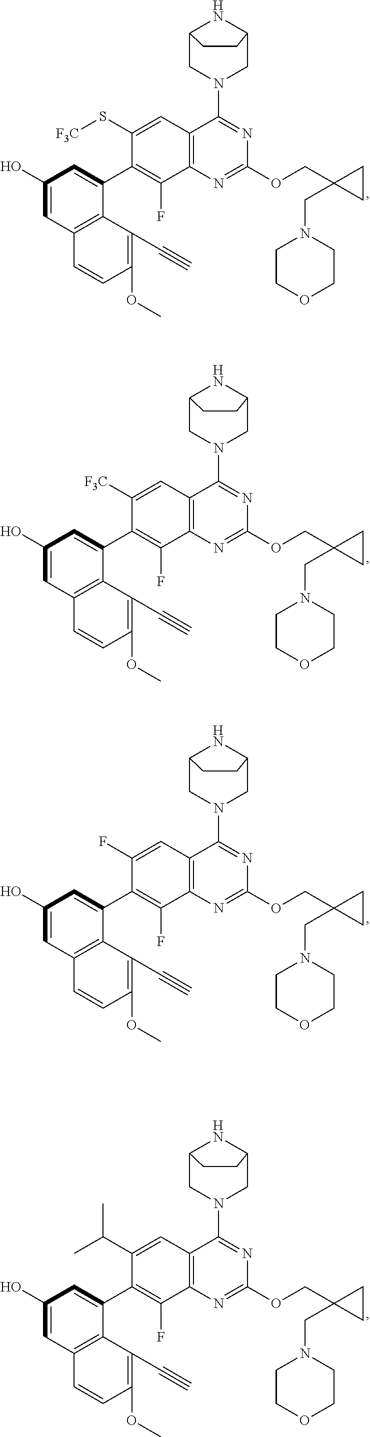

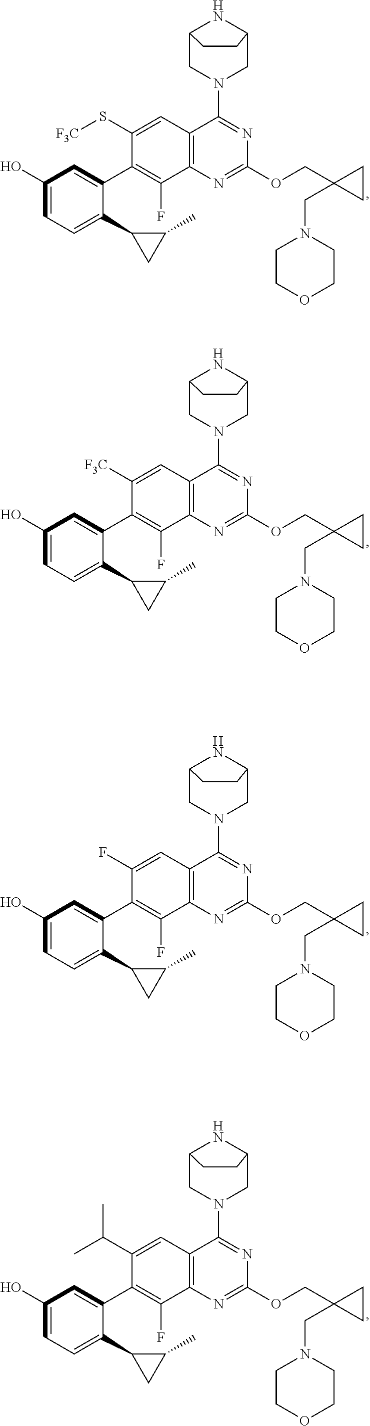

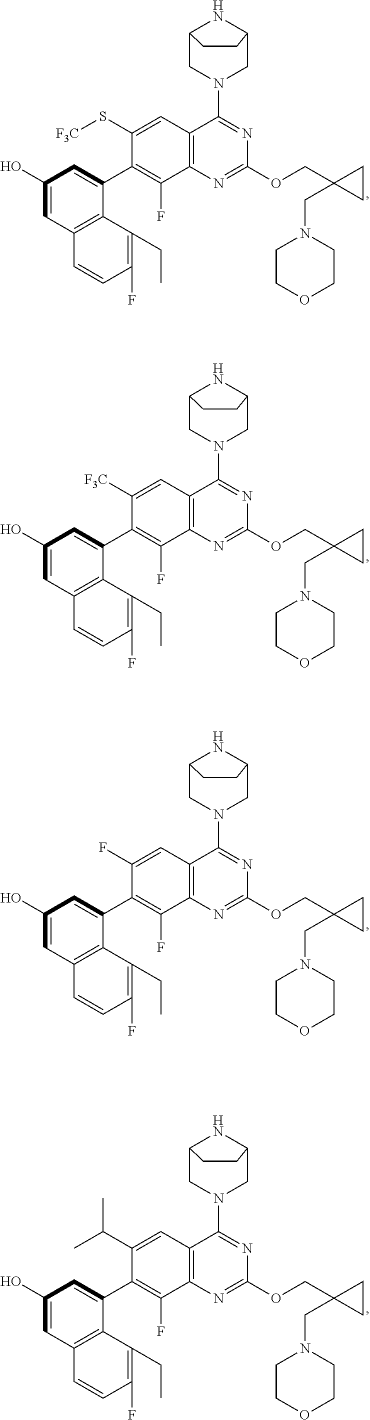

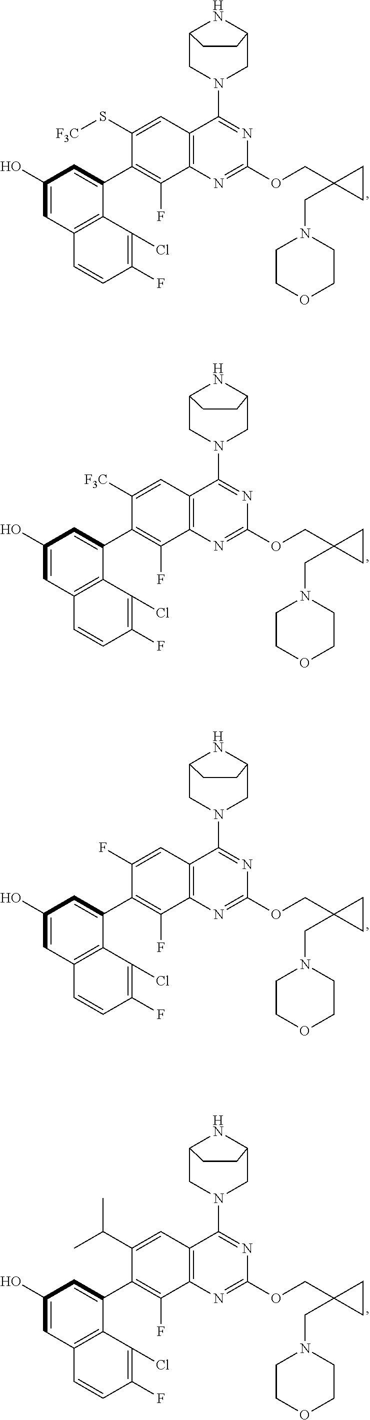

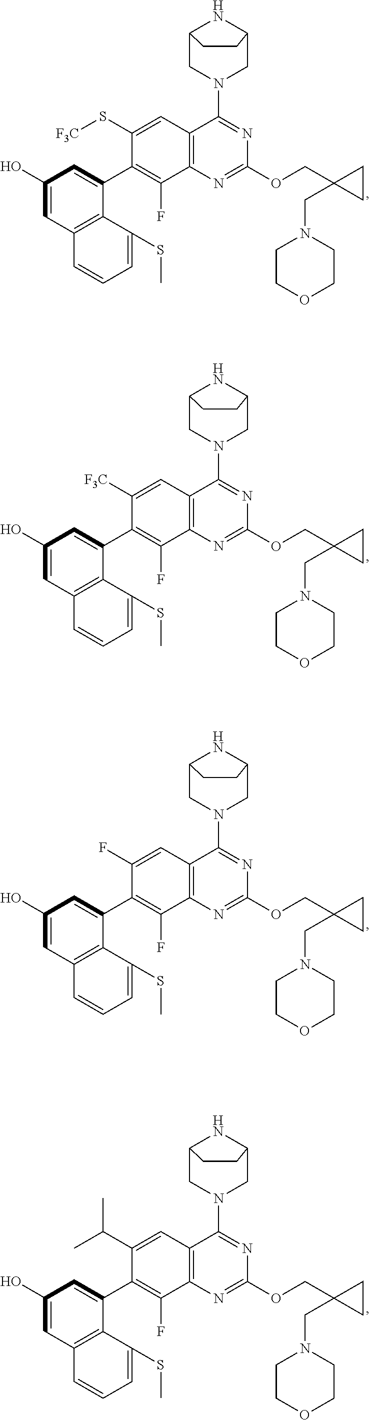

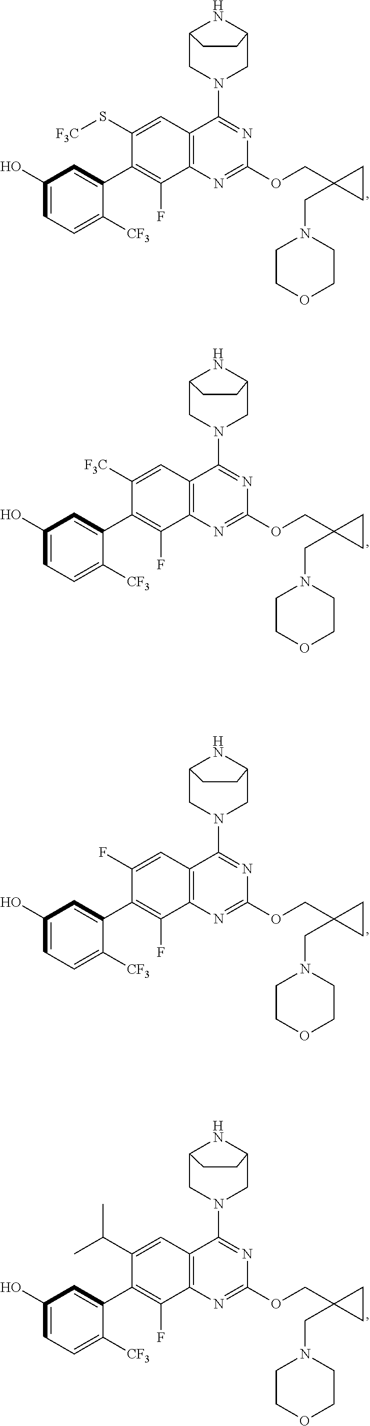

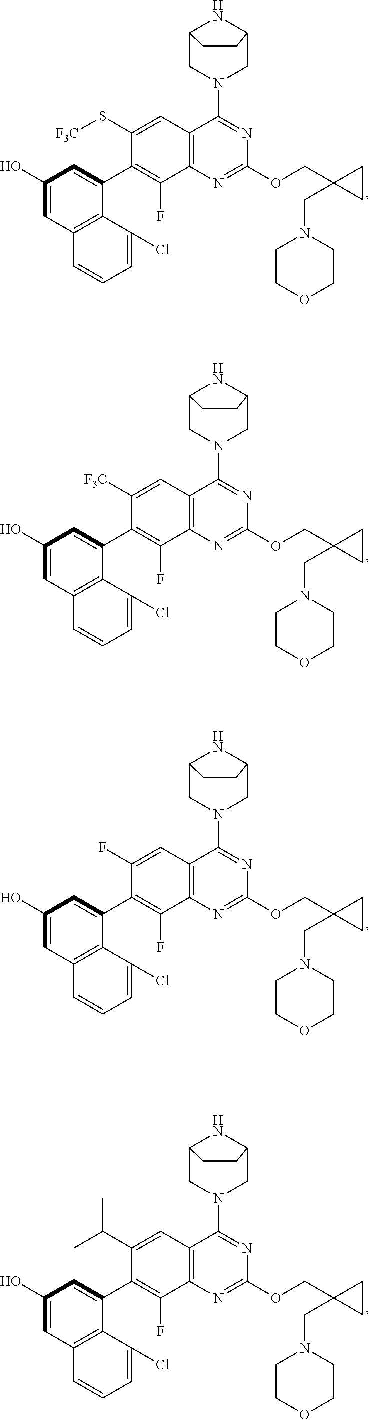

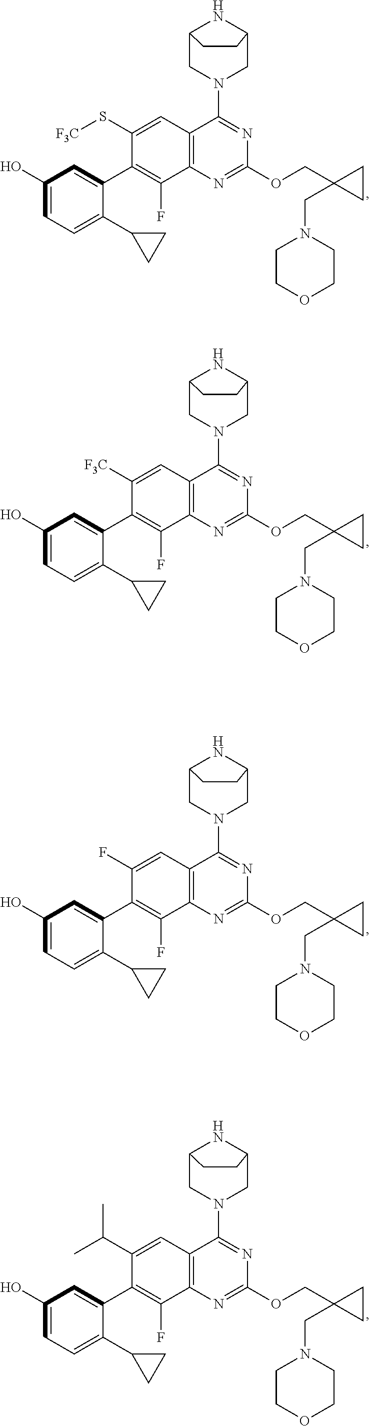

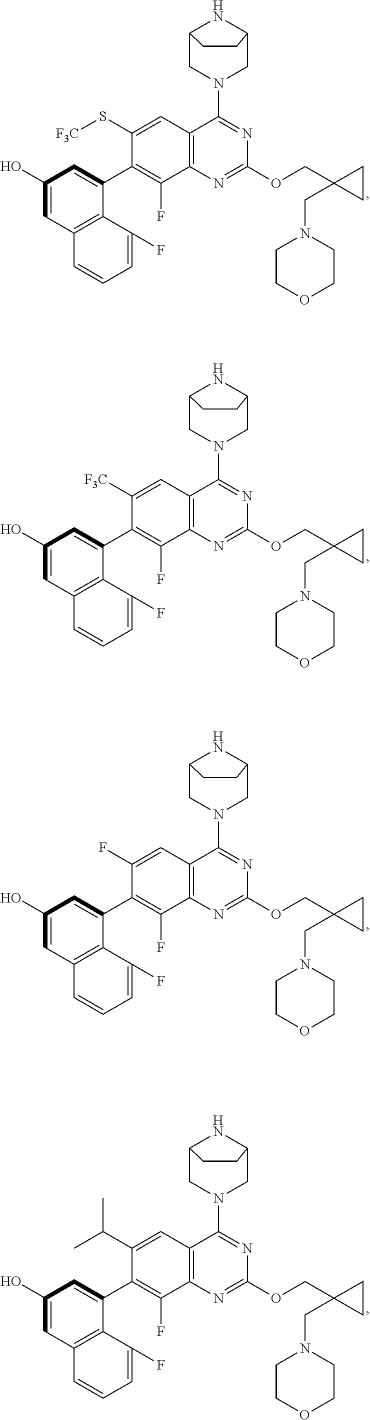

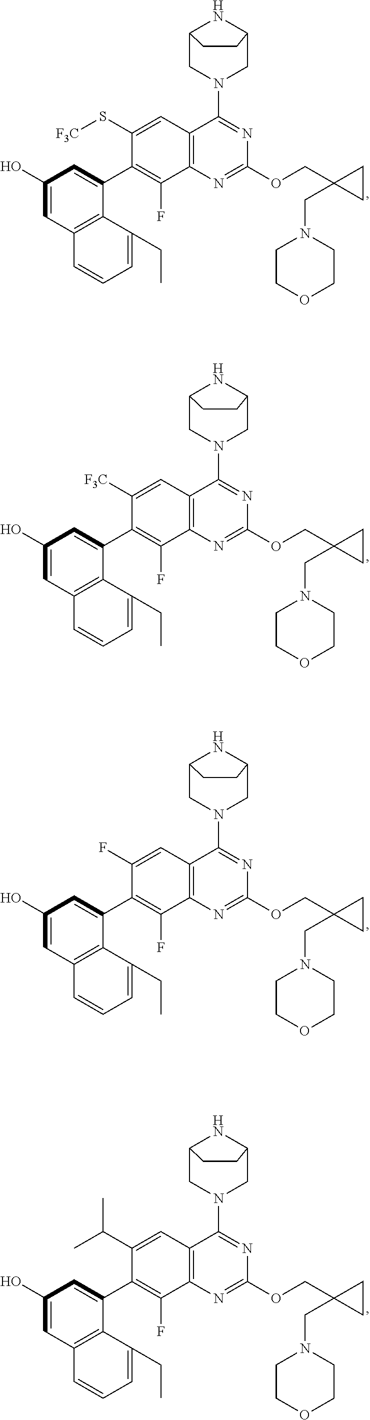

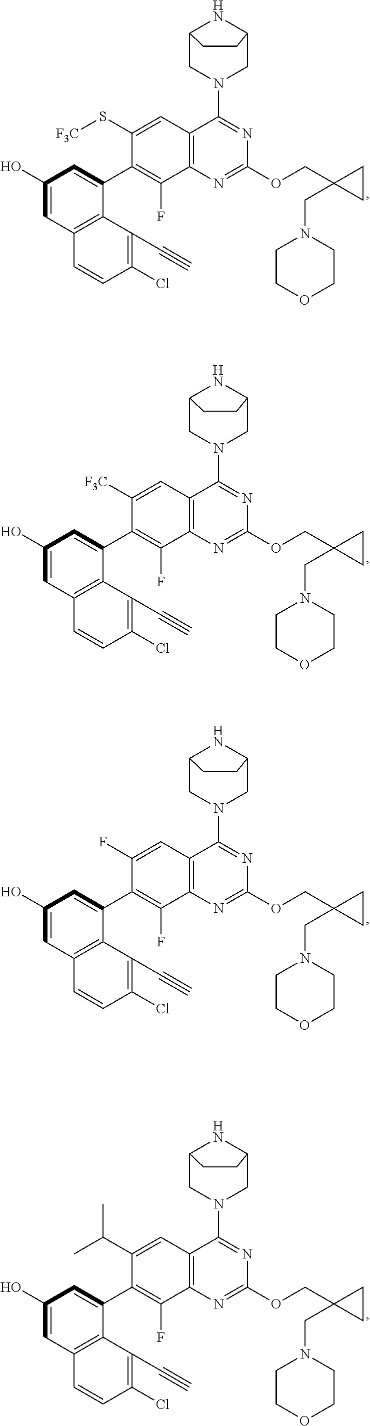

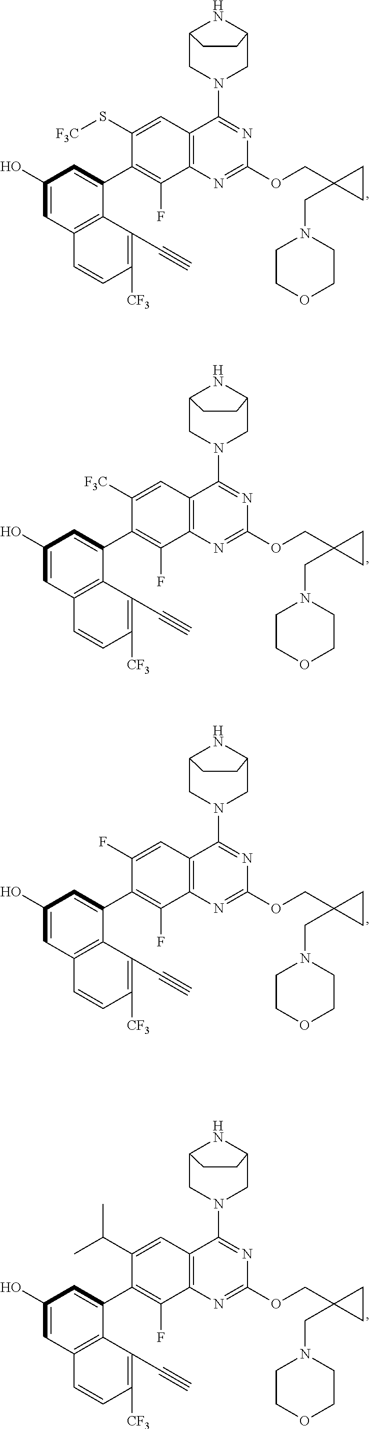

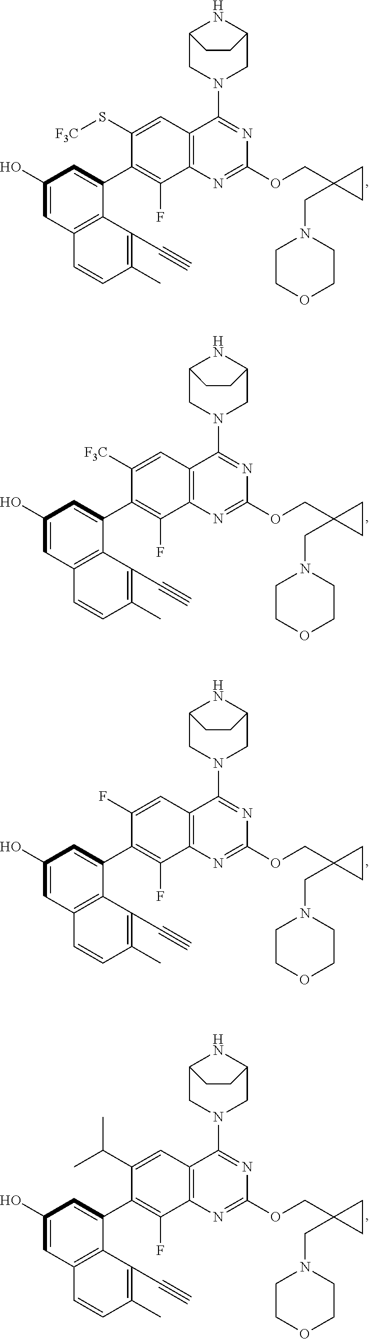

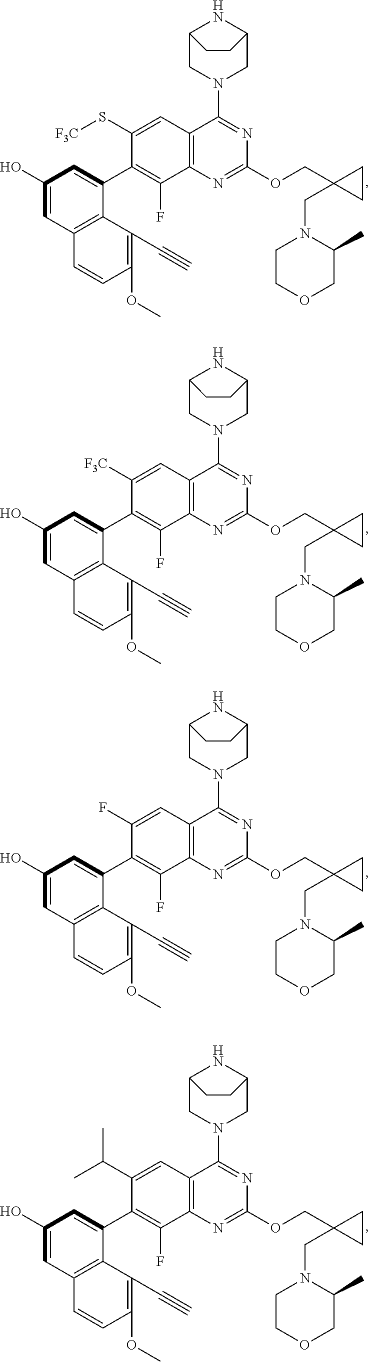

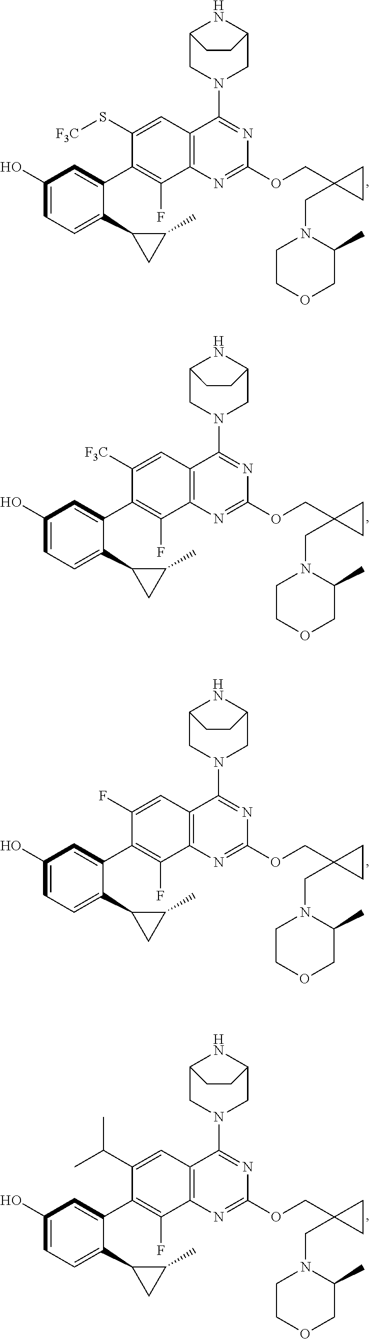

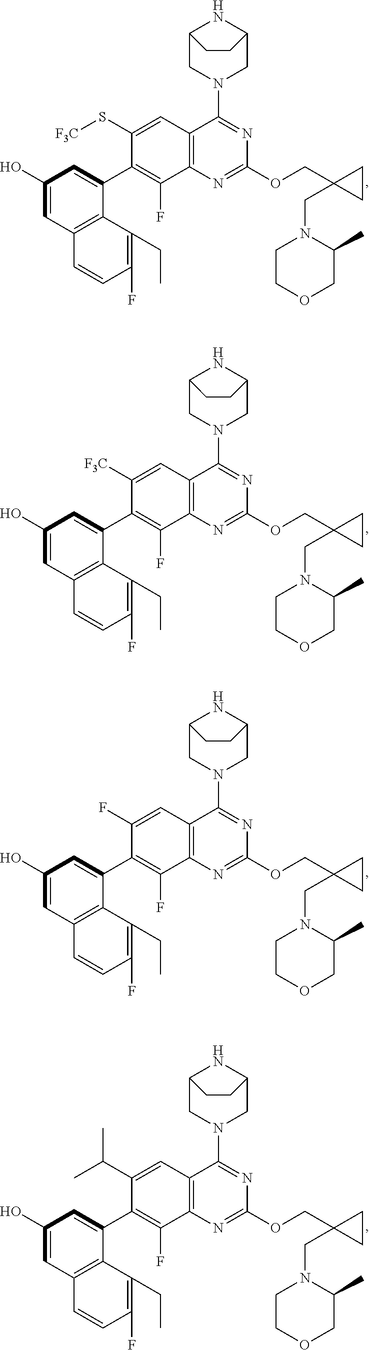

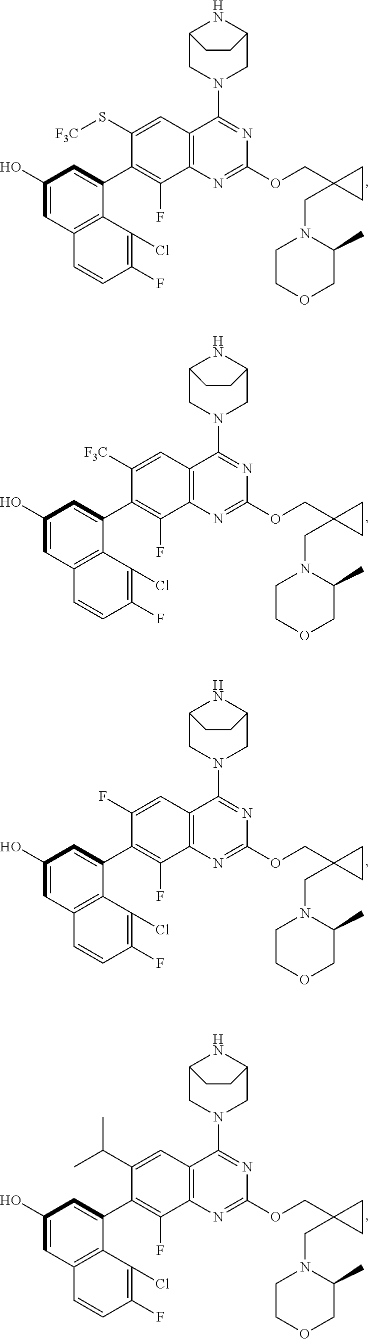

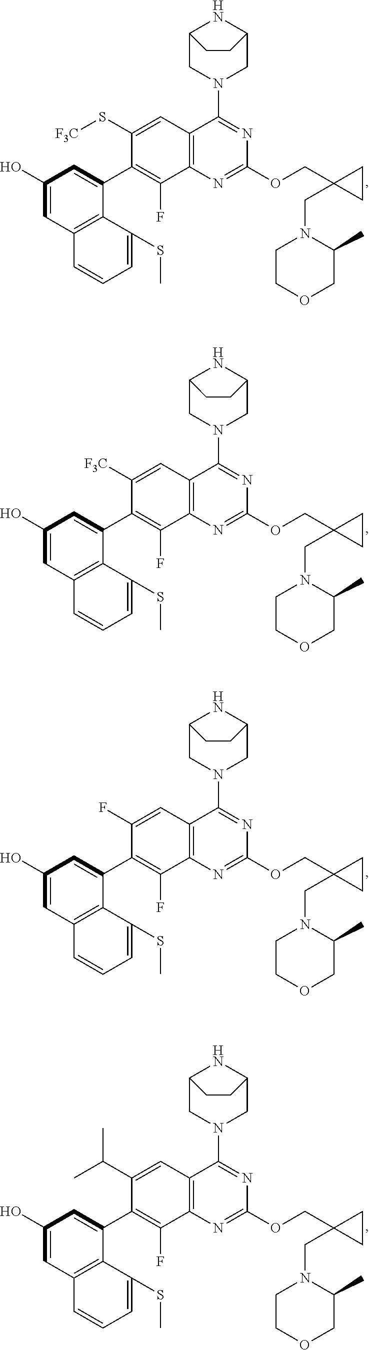

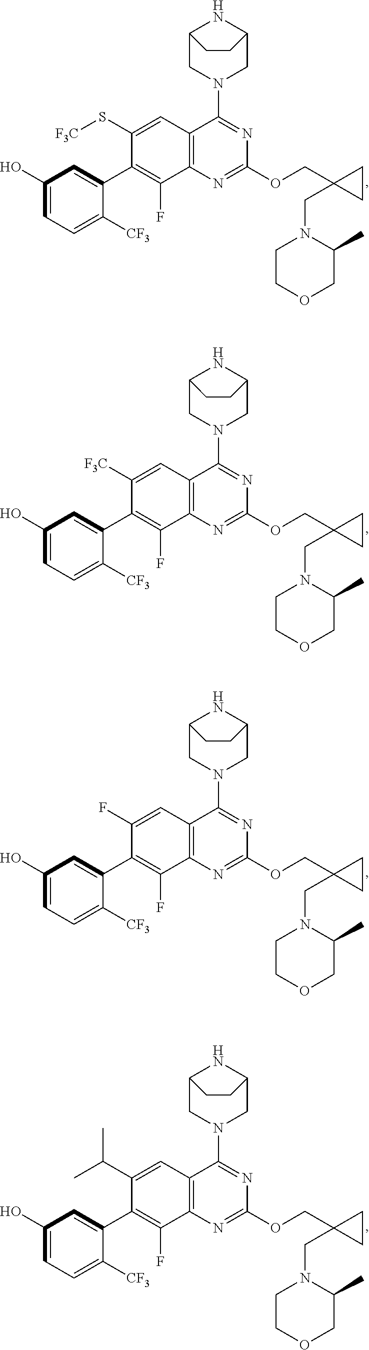

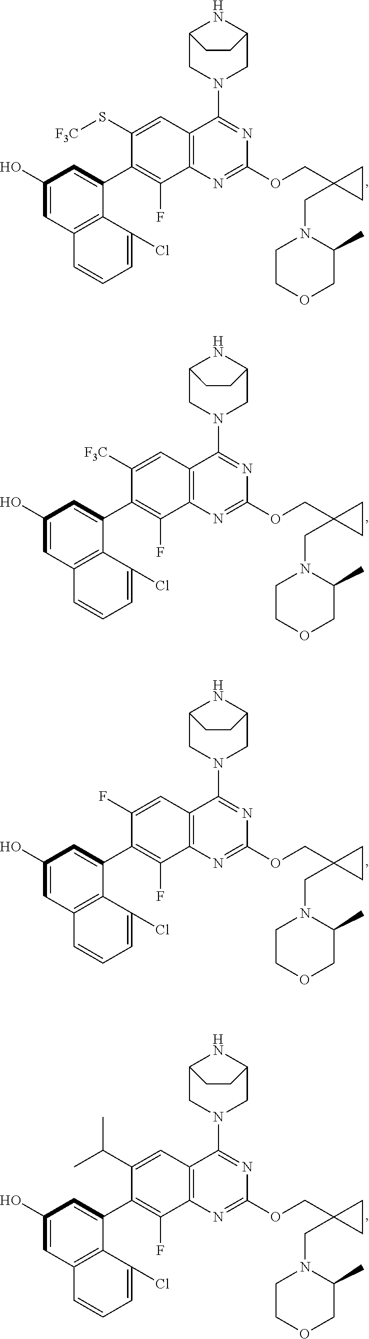

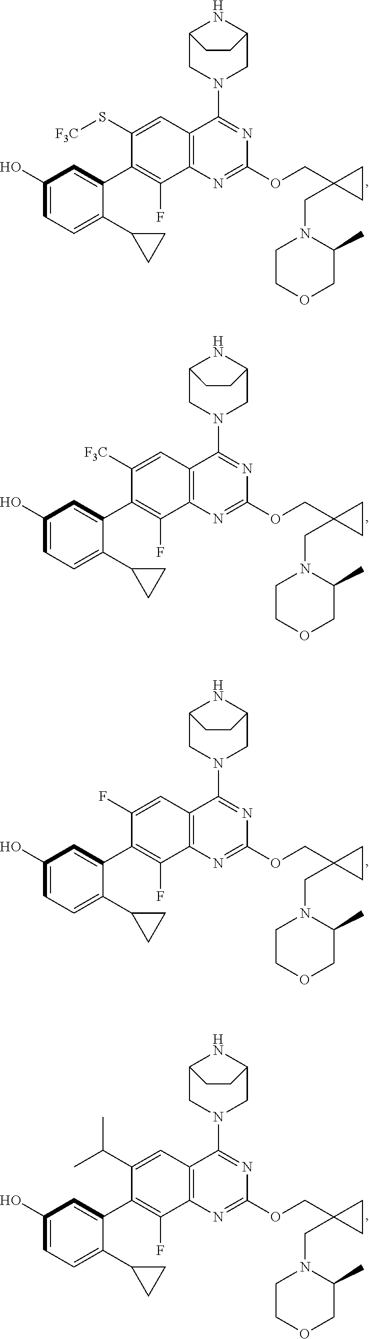

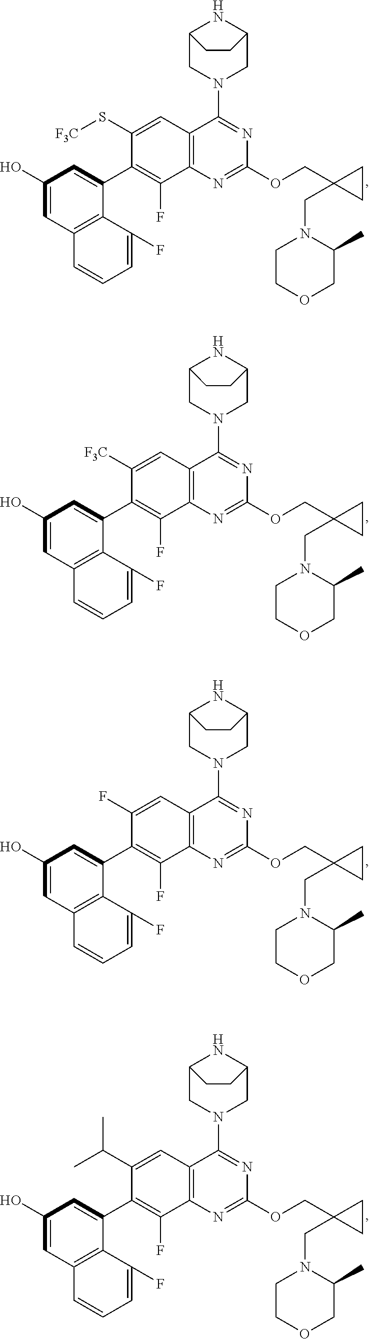

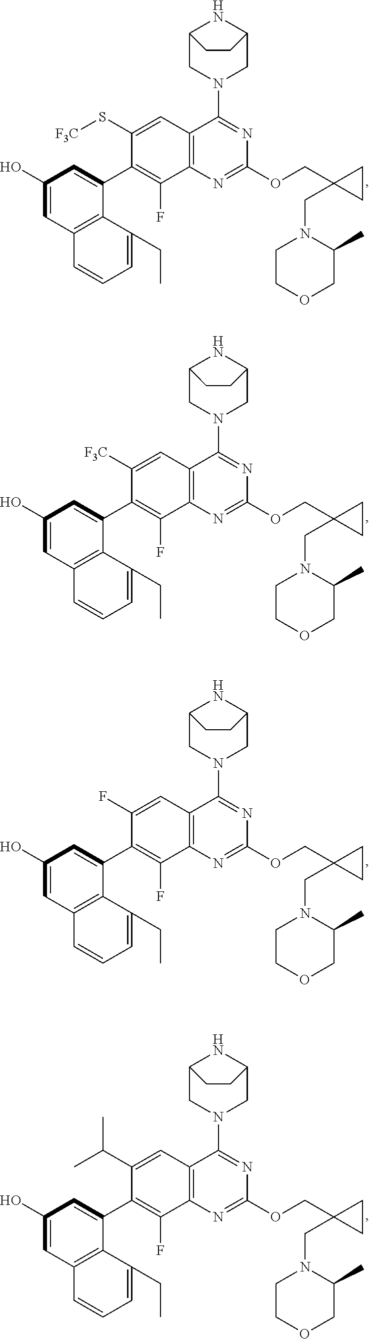

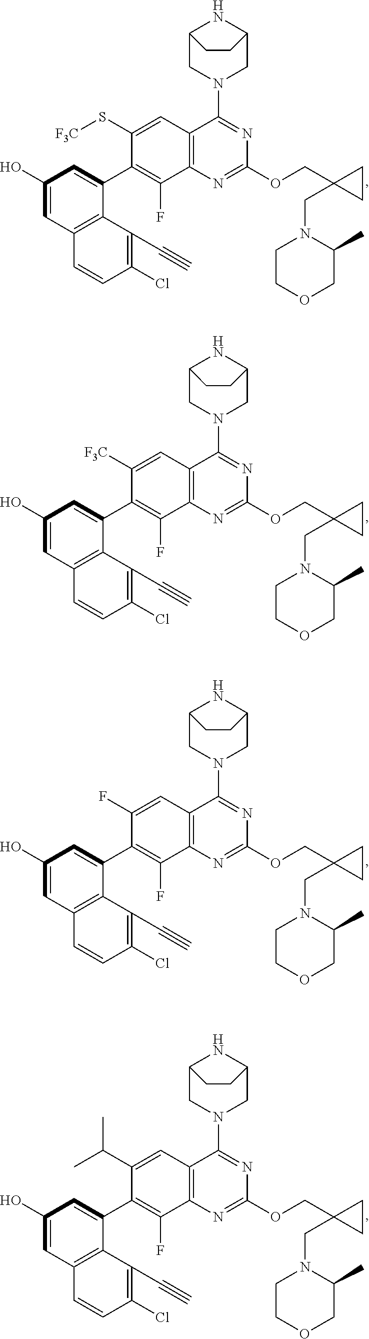

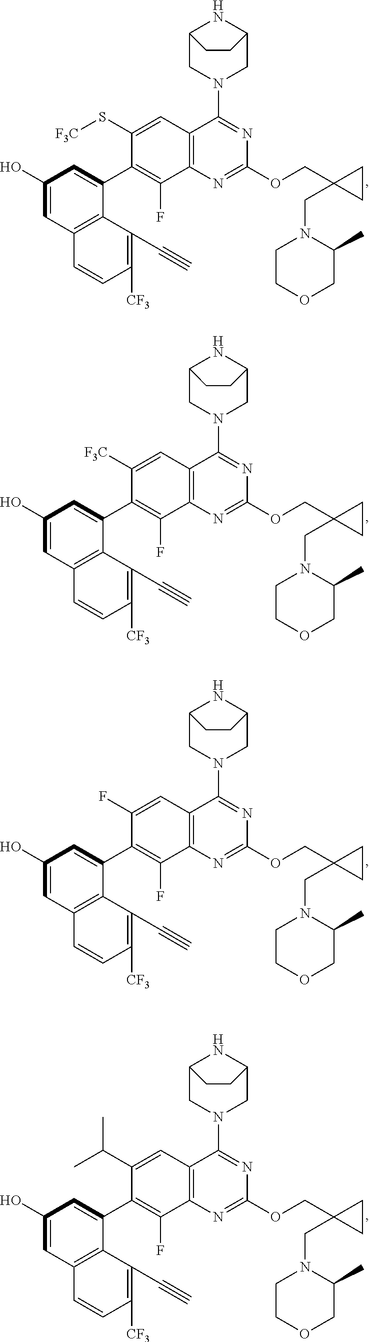

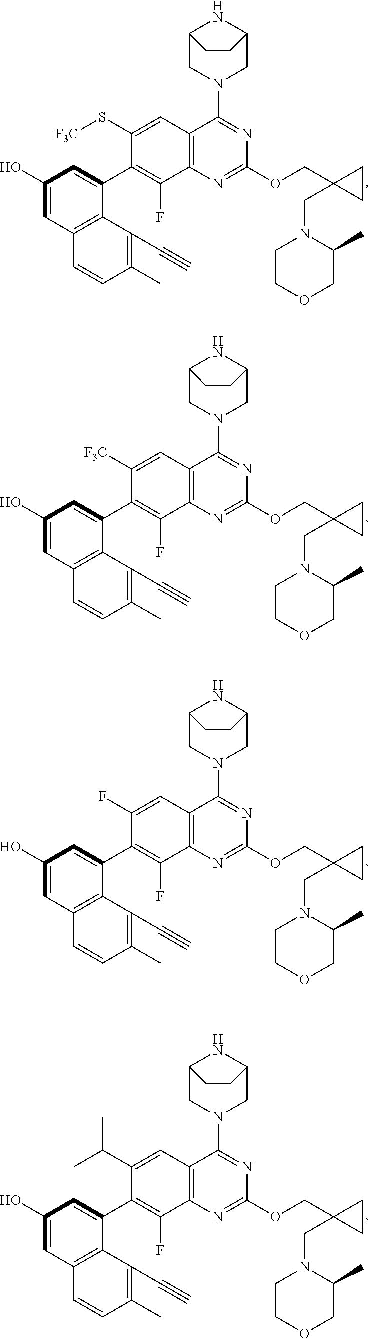

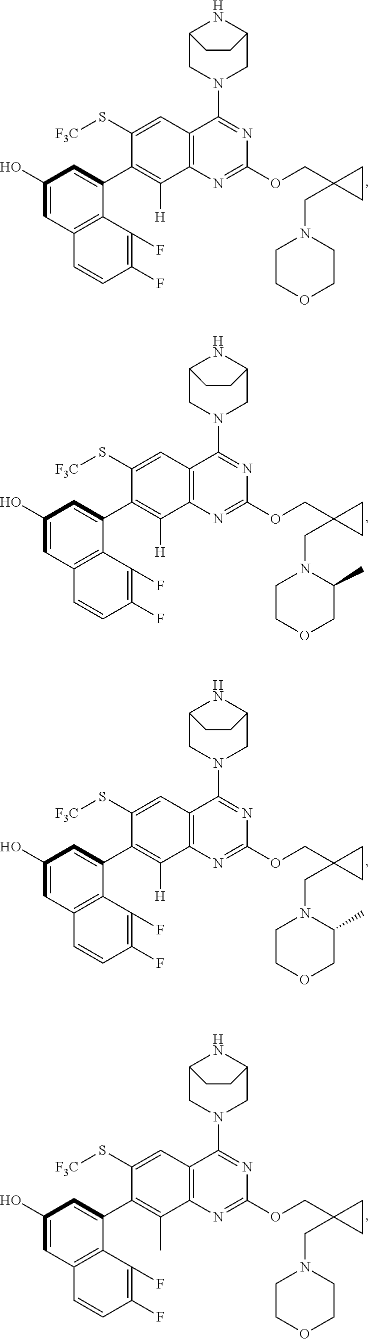

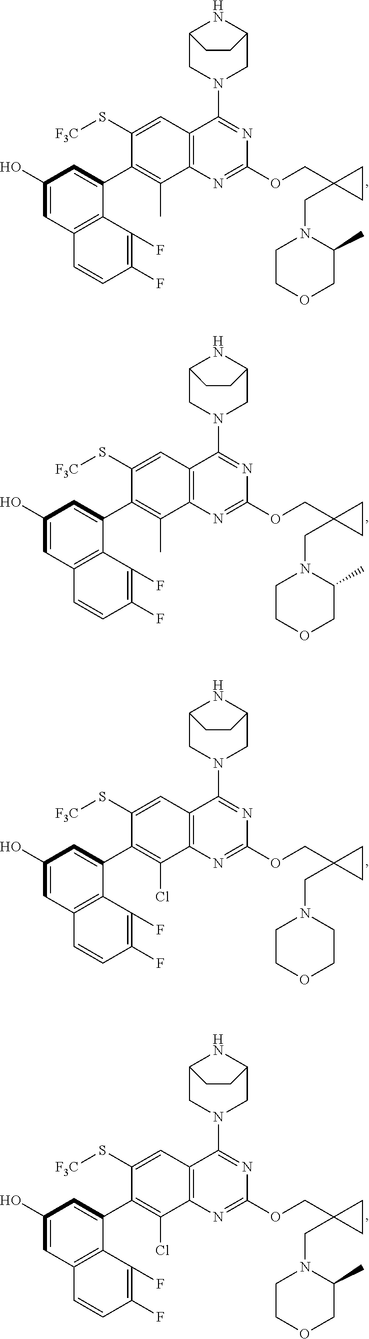

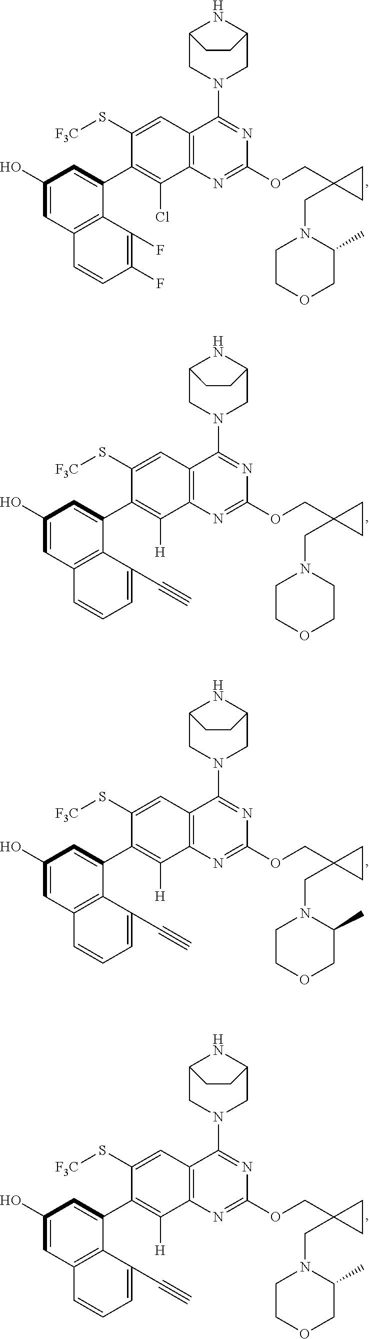

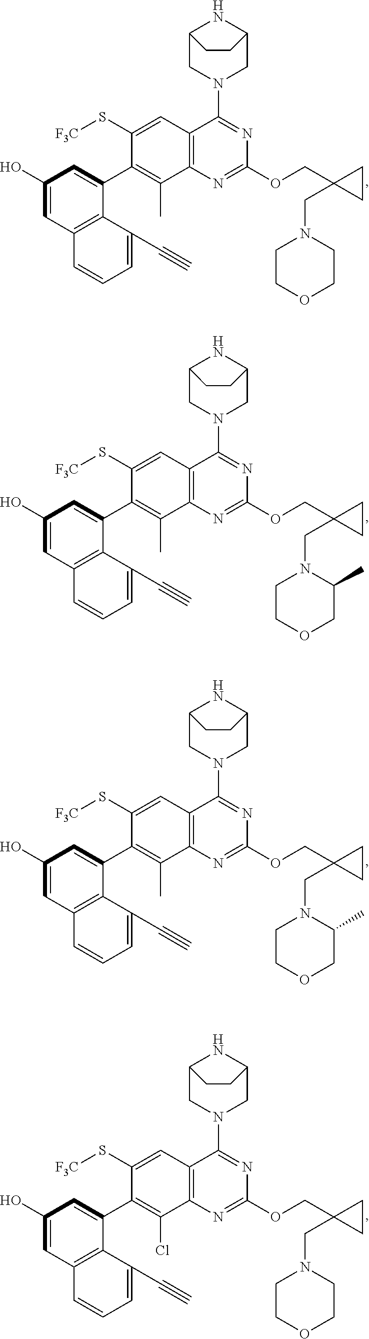

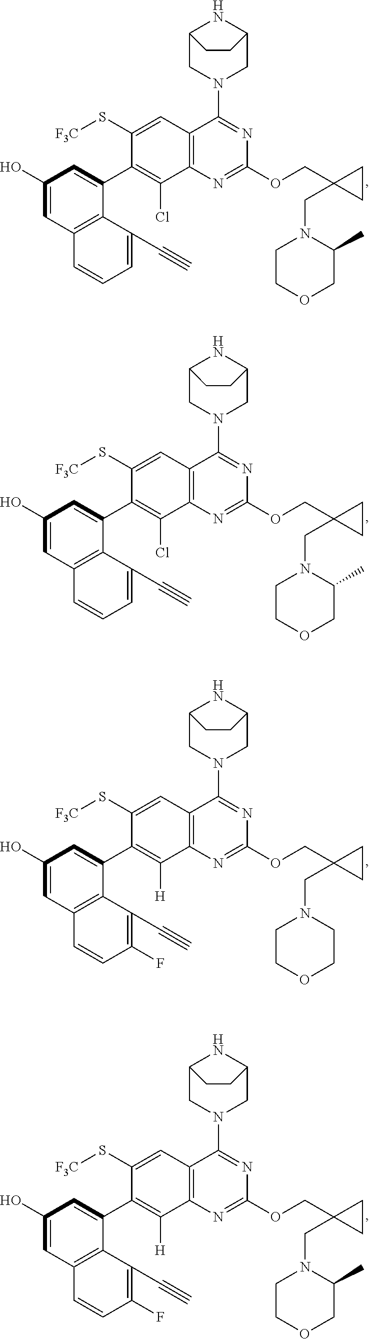

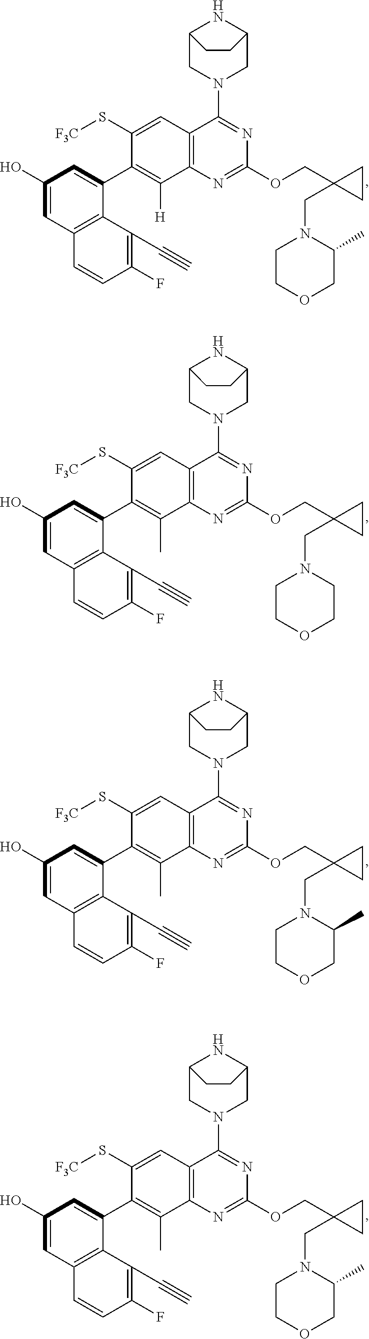

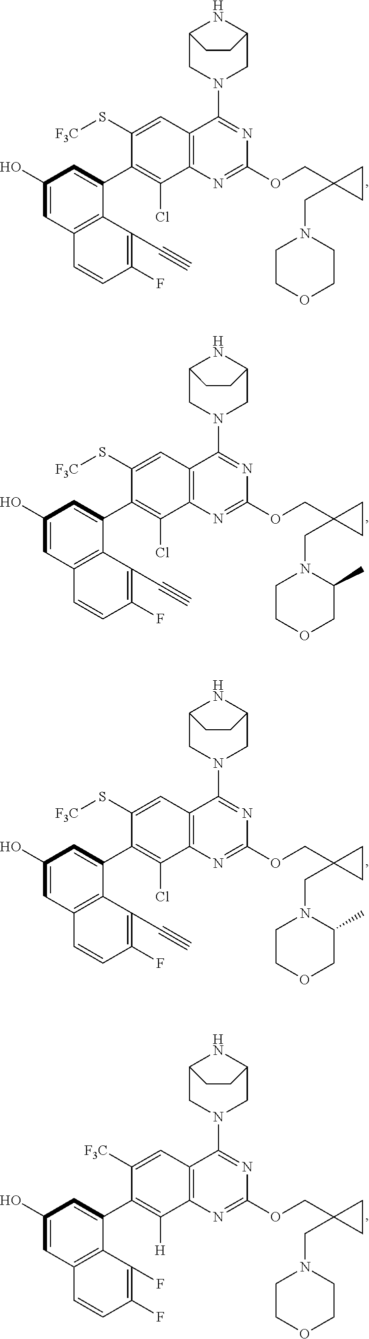

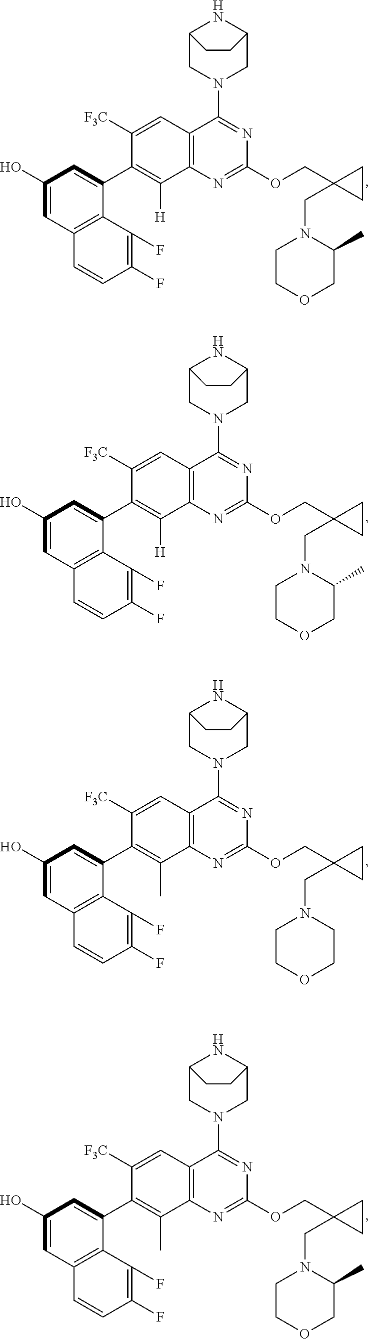

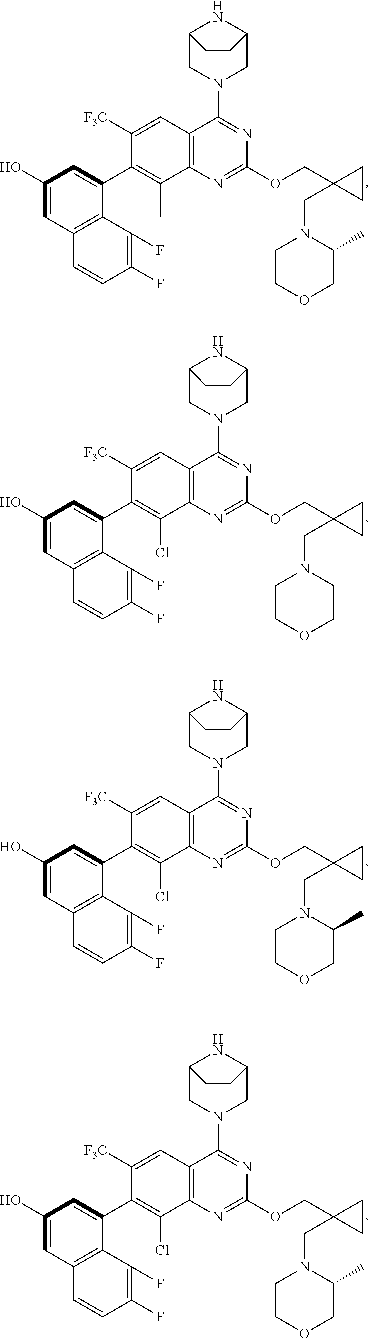

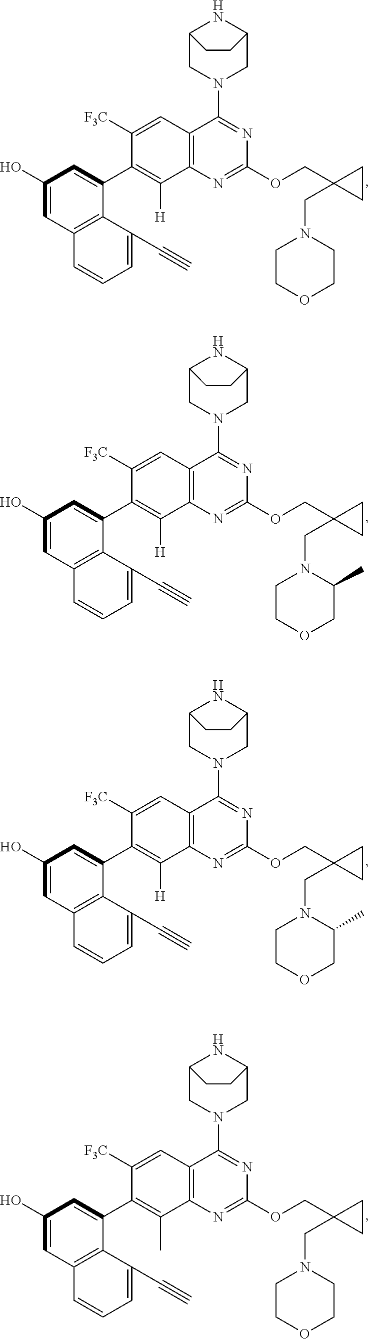

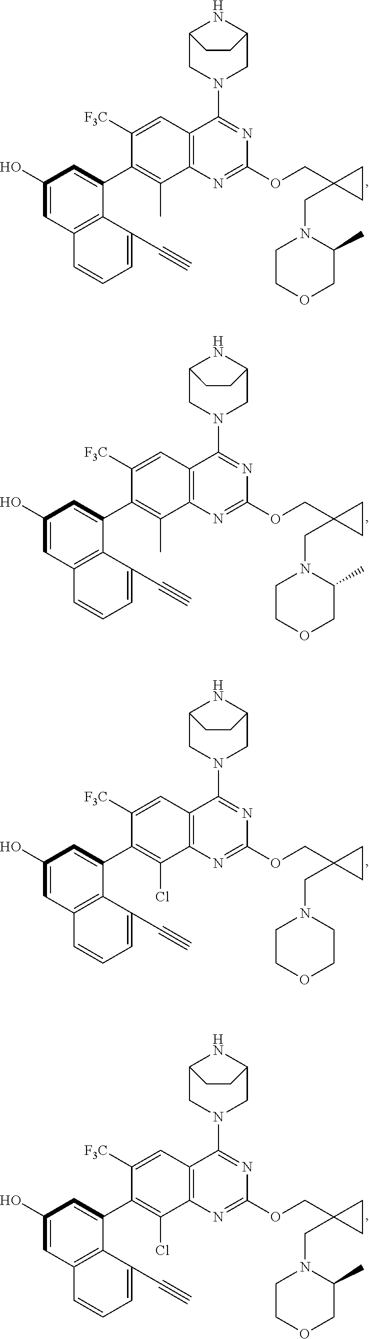

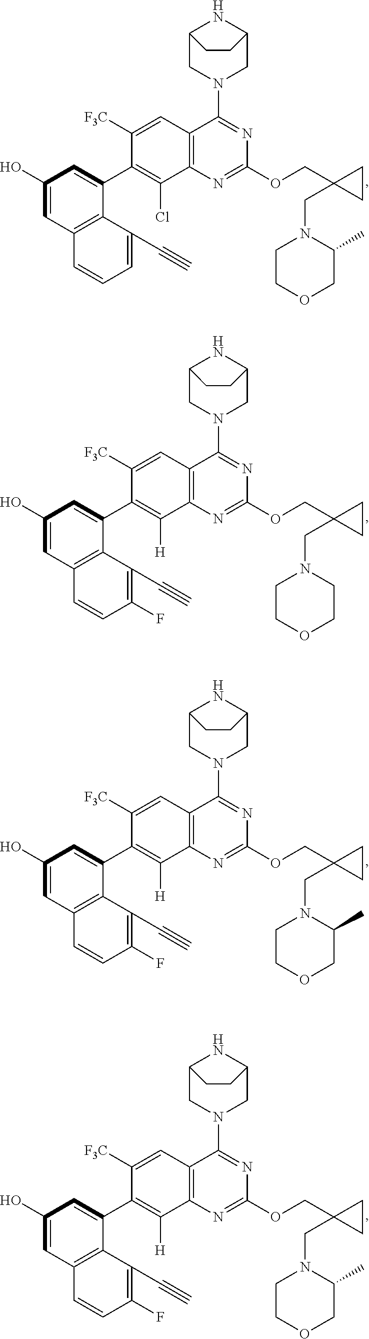

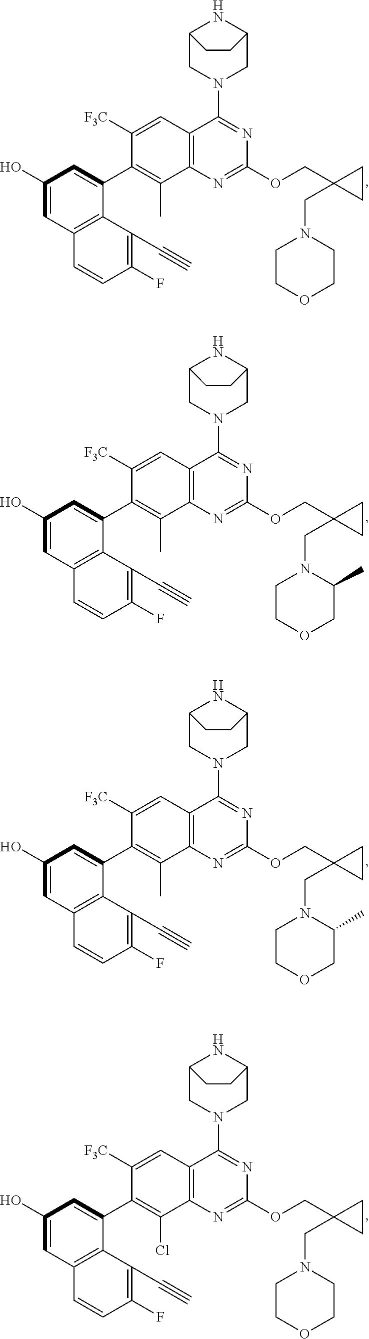

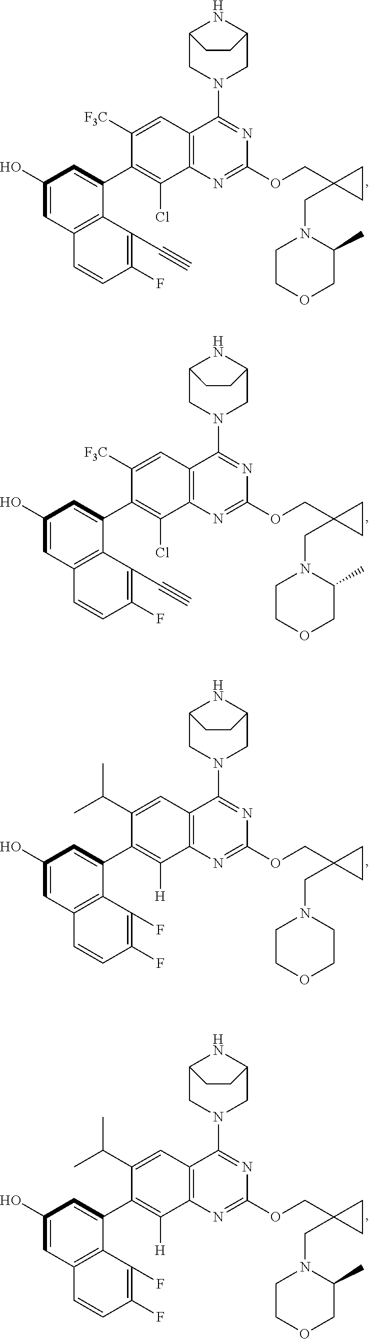

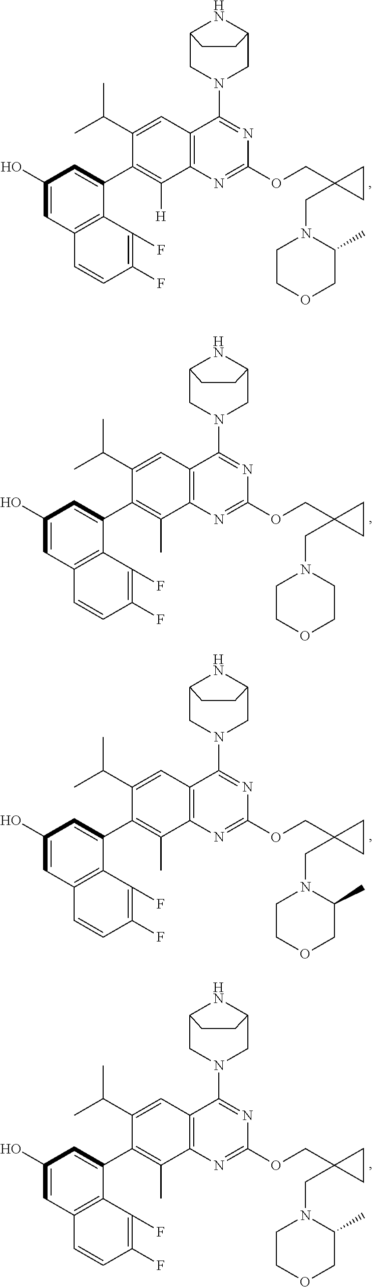

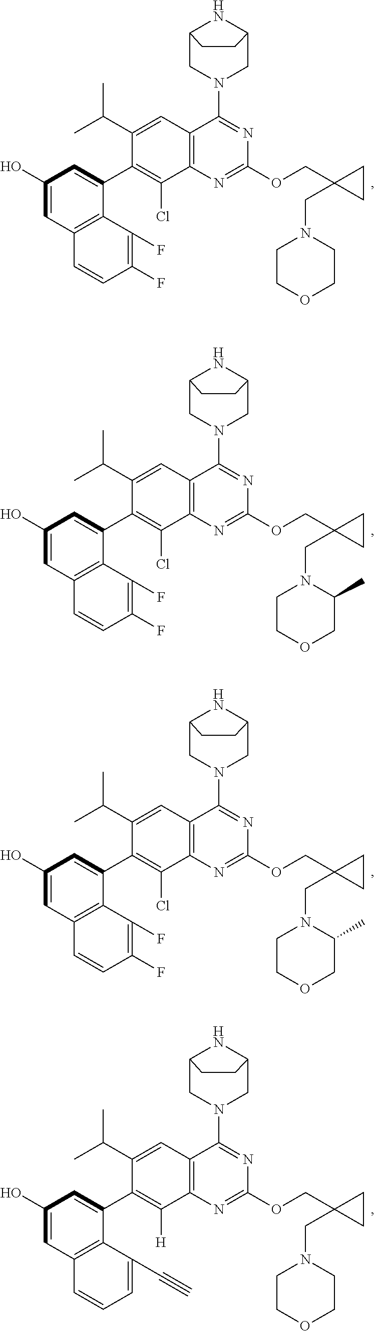

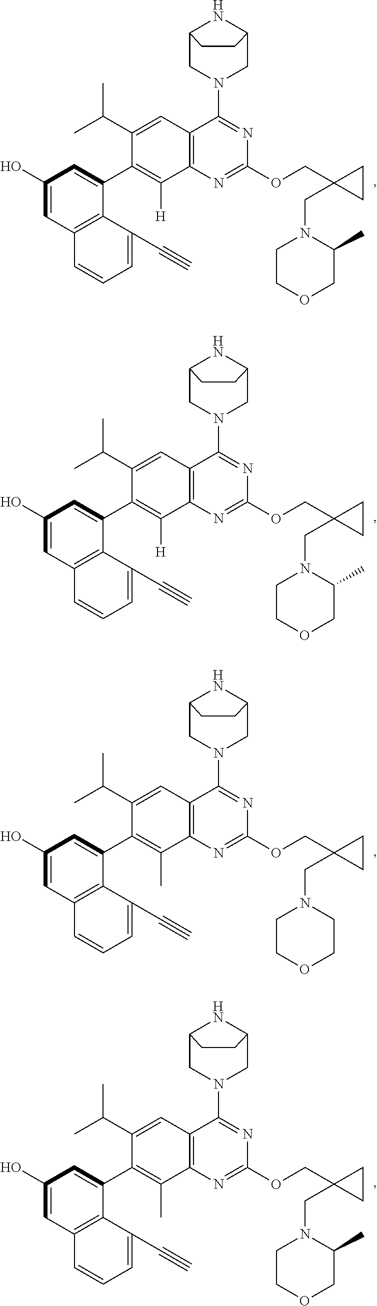

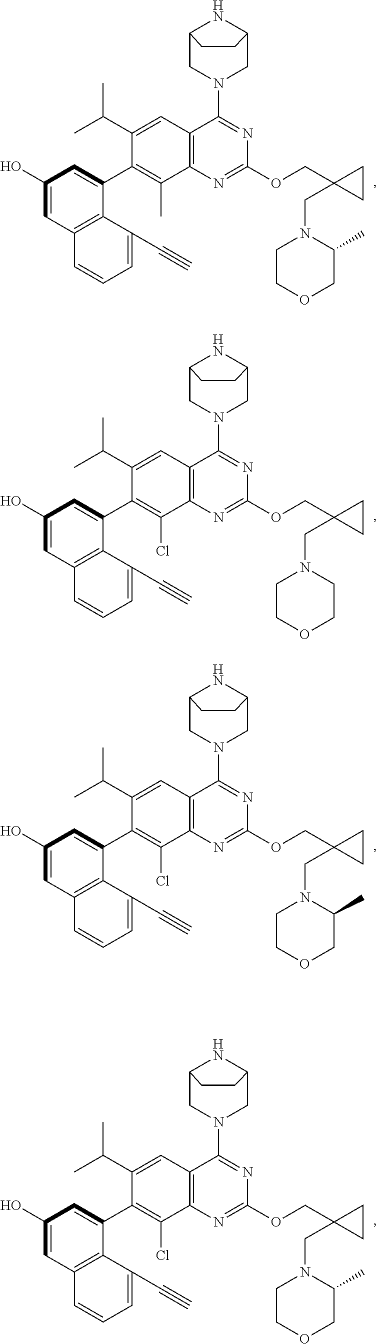

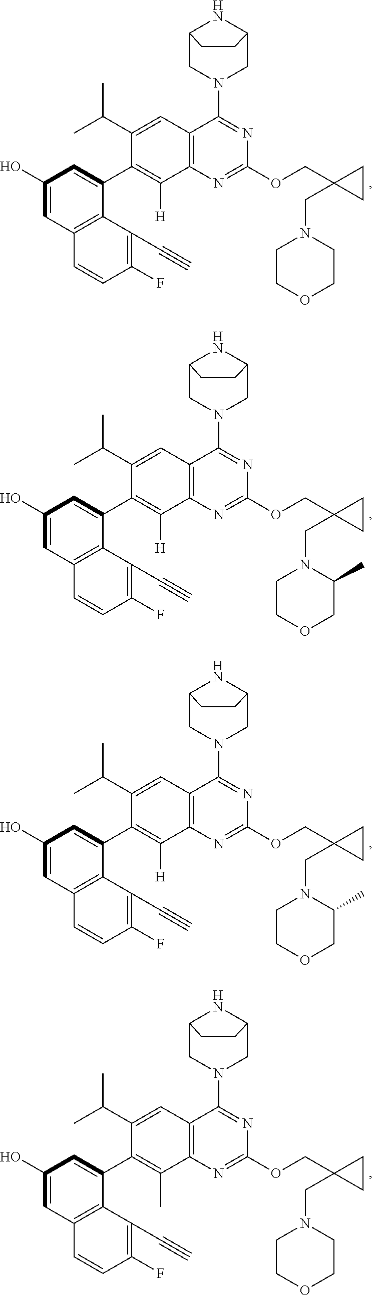

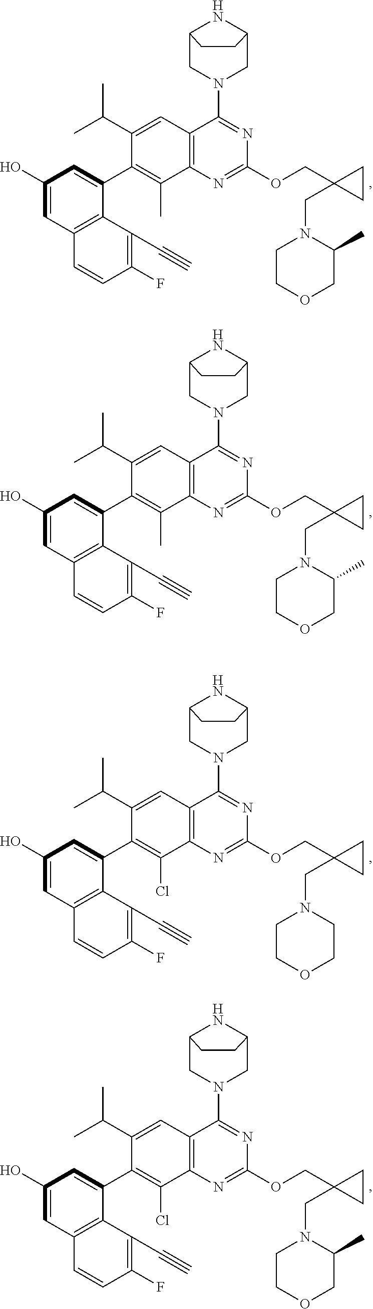

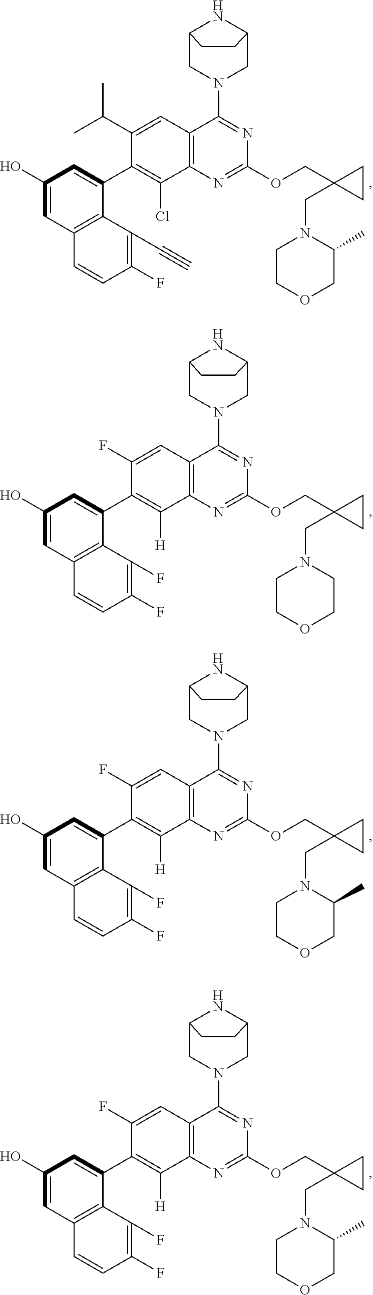

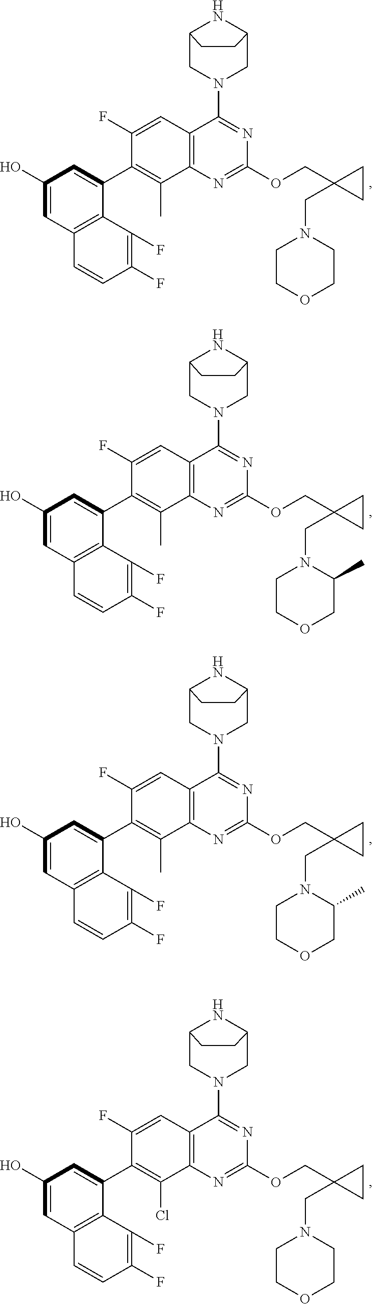

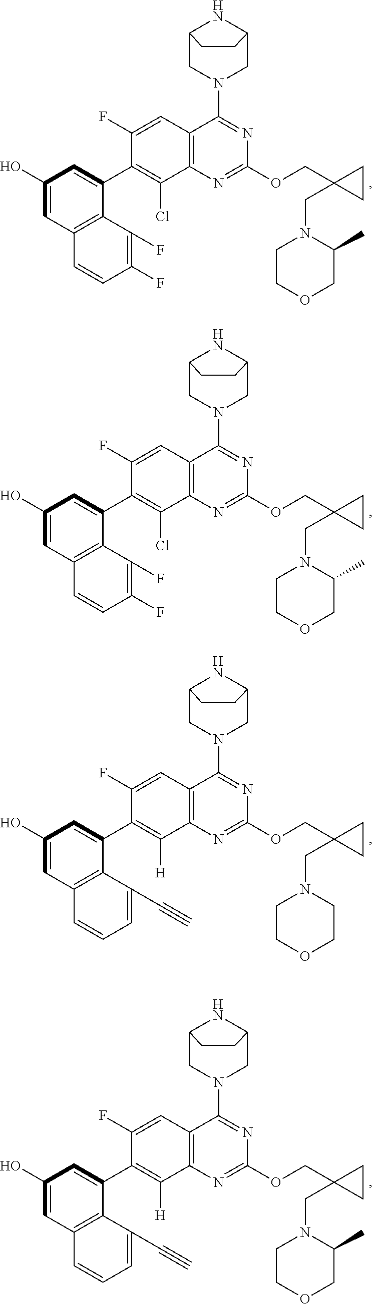

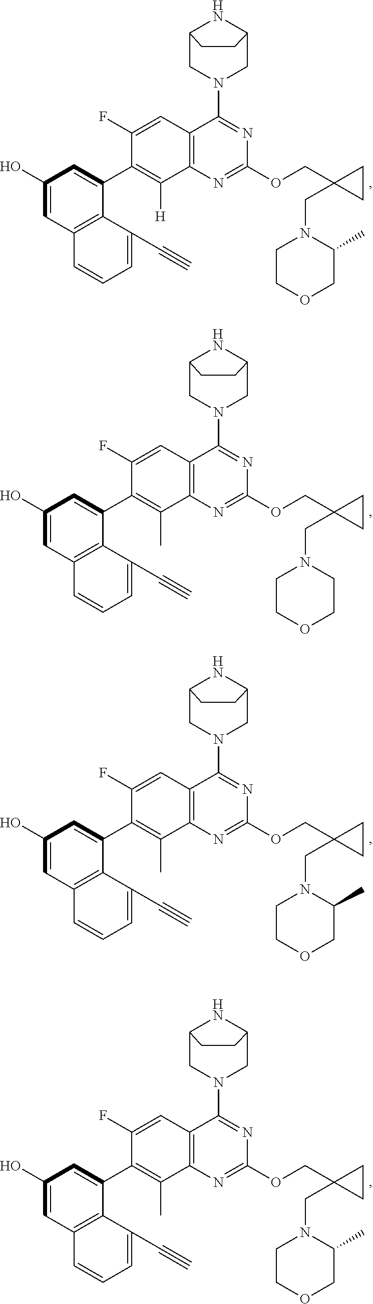

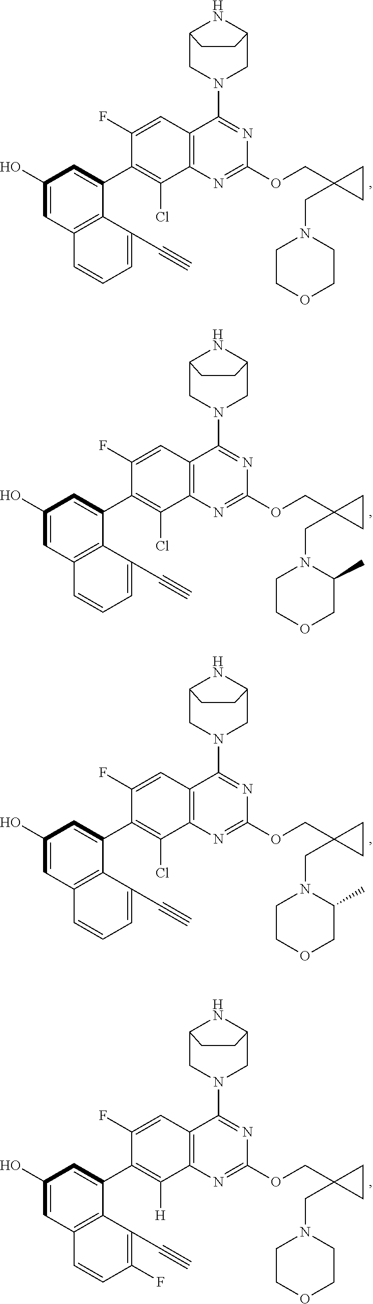

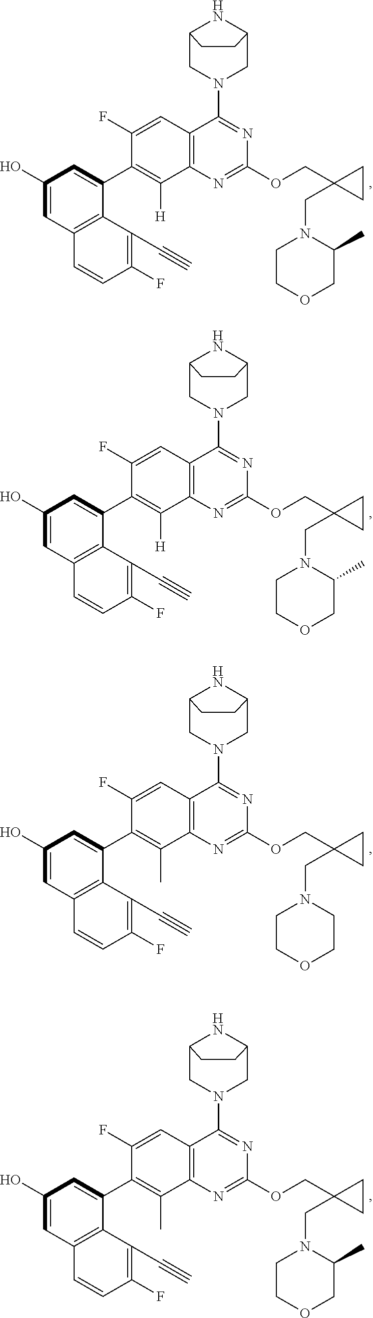

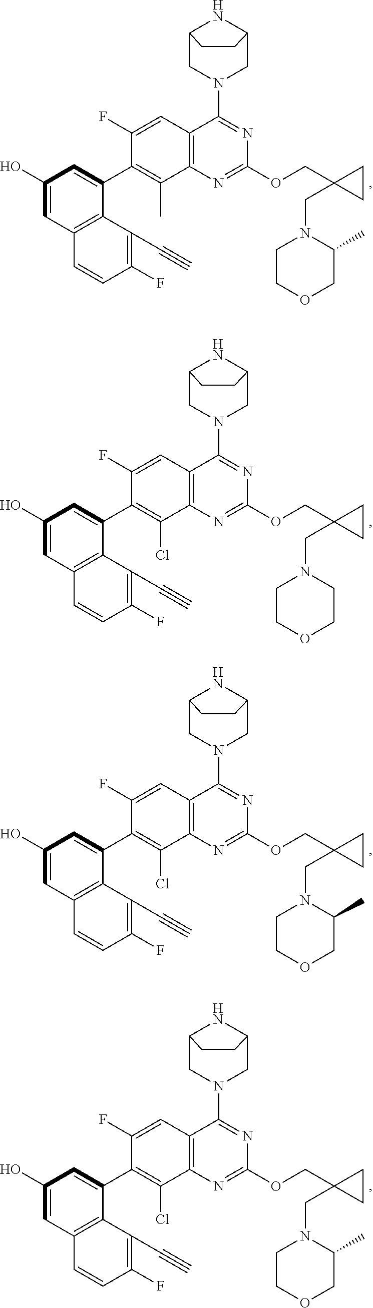

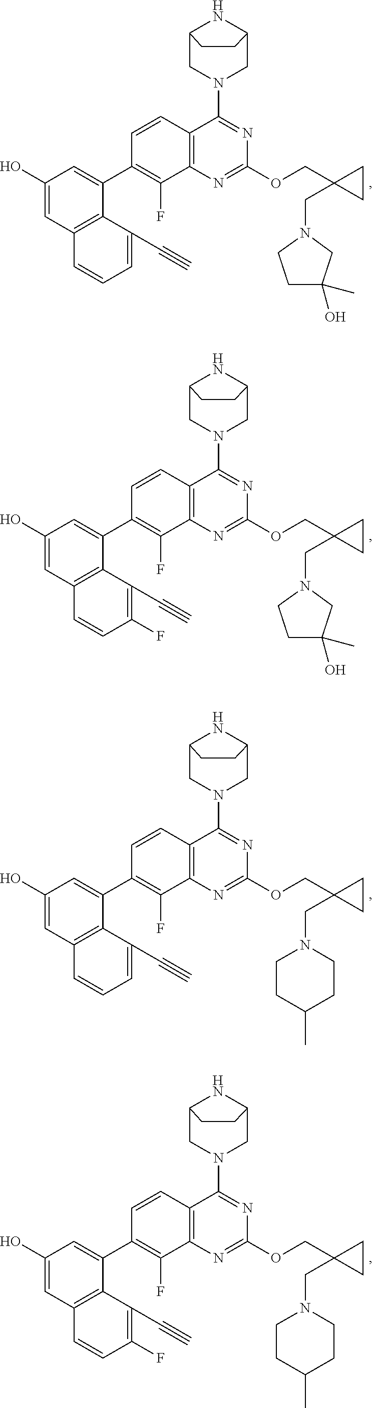 [SMILES: C#Cc1cccc2cc(O)cc(-c3ccc4c(N5CC6CCC(C5)N6)nc(OCC5(CN6CCC(C)(O)C6)CC5)nc4c3F)c12 ; C#Cc1c(F)ccc2cc(O)cc(-c3ccc4c(N5CC6CCC(C5)N6)nc(OCC5(CN6CCC(C)(O)C6)CC5)nc4c3F)c12 ; C#Cc1cccc2cc(O)cc(-c3ccc4c(N5CC6CCC(C5)N6)nc(OCC5(CN6CCC(C)CC6)CC5)nc4c3F)c12 ; C#Cc1c(F)ccc2cc(O)cc(-c3ccc4c(N5CC6CCC(C5)N6)nc(OCC5(CN6CCC(C)CC6)CC5)nc4c3F)c12]

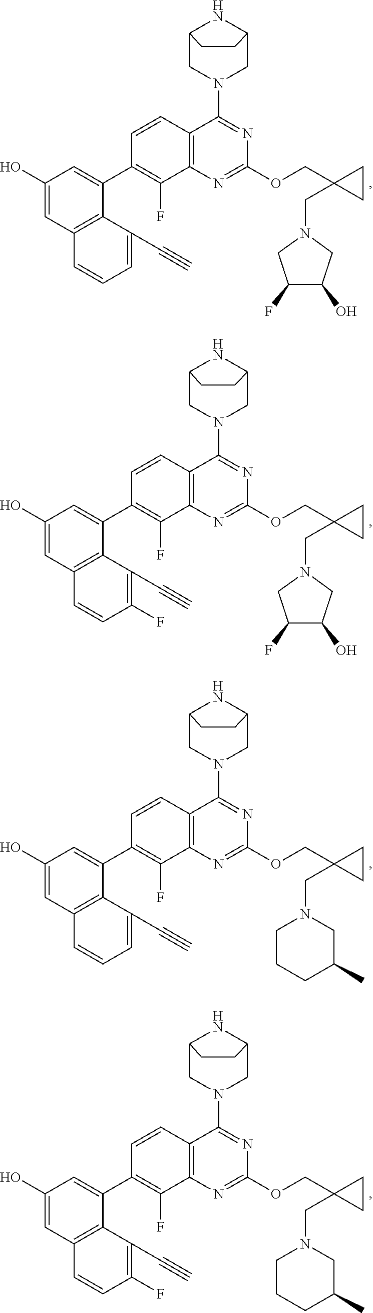 [SMILES: C#Cc1cccc2cc(O)cc(-c3ccc4c(N5CC6CCC(C5)N6)nc(OCC5(CN6C[C@@H](O)[C@@H](F)C6)CC5)nc4c3F)c12 ; C#Cc1c(F)ccc2cc(O)cc(-c3ccc4c(N5CC6CCC(C5)N6)nc(OCC5(CN6C[C@@H](O)[C@@H](F)C6)CC5)nc4c3F)c12 ; C#Cc1cccc2cc(O)cc(-c3ccc4c(N5CC6CCC(C5)N6)nc(OCC5(CN6CCC[C@H](C)C6)CC5)nc4c3F)c12 ; C#Cc1c(F)ccc2cc(O)cc(-c3ccc4c(N5CC6CCC(C5)N6)nc(OCC5(CN6CCC[C@H](C)C6)CC5)nc4c3F)c12]

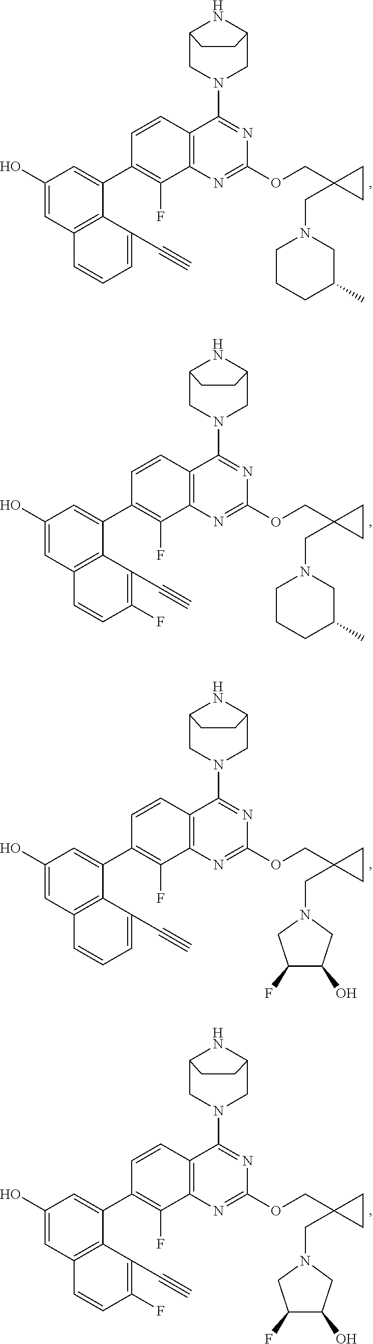 [SMILES: C#Cc1cccc2cc(O)cc(-c3ccc4c(N5CC6CCC(C5)N6)nc(OCC5(CN6CCC[C@@H](C)C6)CC5)nc4c3F)c12 ; C#Cc1c(F)ccc2cc(O)cc(-c3ccc4c(N5CC6CCC(C5)N6)nc(OCC5(CN6CCC[C@@H](C)C6)CC5)nc4c3F)c12 ; C#Cc1cccc2cc(O)cc(-c3ccc4c(N5CC6CCC(C5)N6)nc(OCC5(CN6C[C@@H](O)[C@@H](F)C6)CC5)nc4c3F)c12 ; C#Cc1c(F)ccc2cc(O)cc(-c3ccc4c(N5CC6CCC(C5)N6)nc(OCC5(CN6C[C@@H](O)[C@@H](F)C6)CC5)nc4c3F)c12]

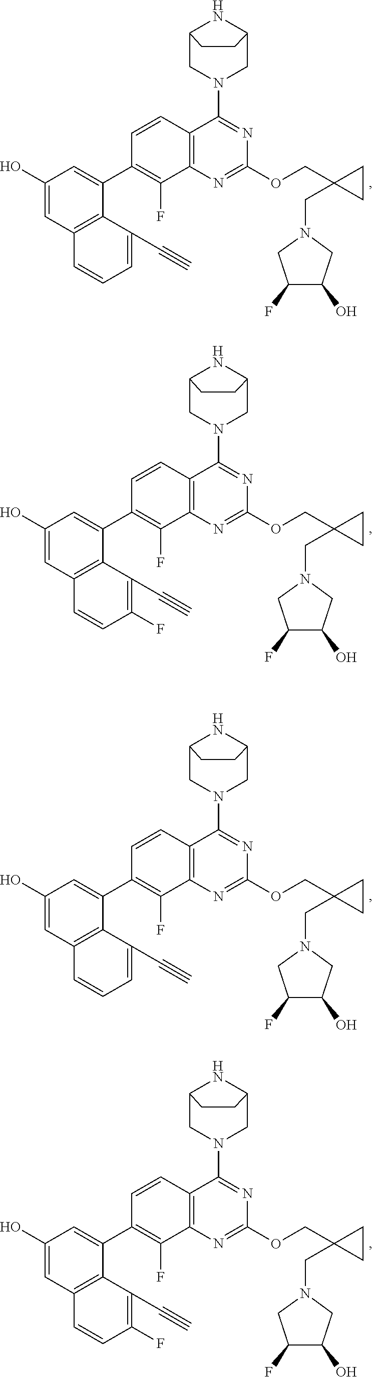 [SMILES: C#Cc1cccc2cc(O)cc(-c3ccc4c(N5CC6CCC(C5)N6)nc(OCC5(CN6C[C@@H](O)[C@@H](F)C6)CC5)nc4c3F)c12 ; C#Cc1c(F)ccc2cc(O)cc(-c3ccc4c(N5CC6CCC(C5)N6)nc(OCC5(CN6C[C@@H](O)[C@@H](F)C6)CC5)nc4c3F)c12 ; C#Cc1cccc2cc(O)cc(-c3ccc4c(N5CC6CCC(C5)N6)nc(OCC5(CN6C[C@@H](O)[C@@H](F)C6)CC5)nc4c3F)c12 ; C#Cc1c(F)ccc2cc(O)cc(-c3ccc4c(N5CC6CCC(C5)N6)nc(OCC5(CN6C[C@@H](O)[C@@H](F)C6)CC5)nc4c3F)c12]

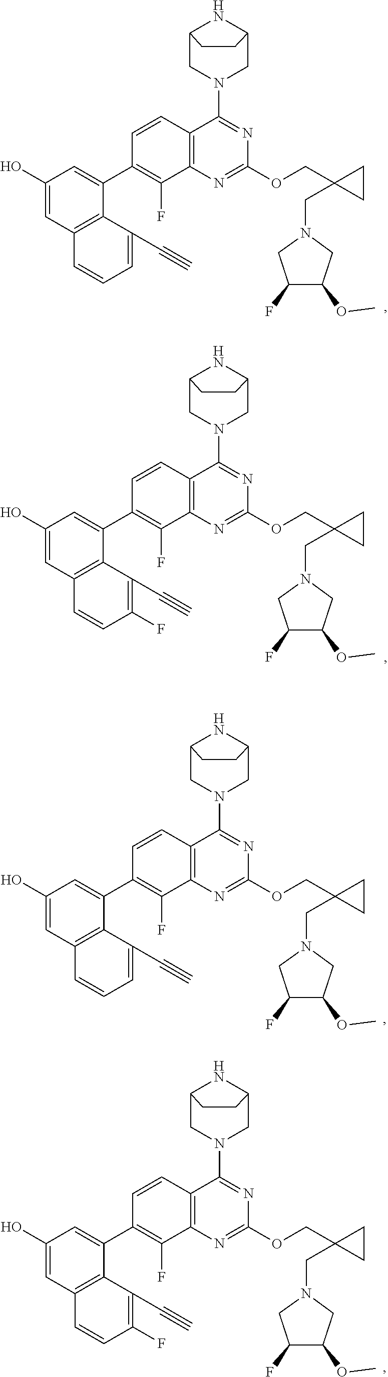 [SMILES: C#Cc1cccc2cc(O)cc(-c3ccc4c(N5CC6CCC(C5)N6)nc(OCC5(CN6C[C@H](F)[C@H](OC)C6)CC5)nc4c3F)c12 ; C#Cc1c(F)ccc2cc(O)cc(-c3ccc4c(N5CC6CCC(C5)N6)nc(OCC5(CN6C[C@H](F)[C@H](OC)C6)CC5)nc4c3F)c12 ; C#Cc1cccc2cc(O)cc(-c3ccc4c(N5CC6CCC(C5)N6)nc(OCC5(CN6C[C@H](F)[C@H](OC)C6)CC5)nc4c3F)c12 ; C#Cc1c(F)ccc2cc(O)cc(-c3ccc4c(N5CC6CCC(C5)N6)nc(OCC5(CN6C[C@H](F)[C@H](OC)C6)CC5)nc4c3F)c12]

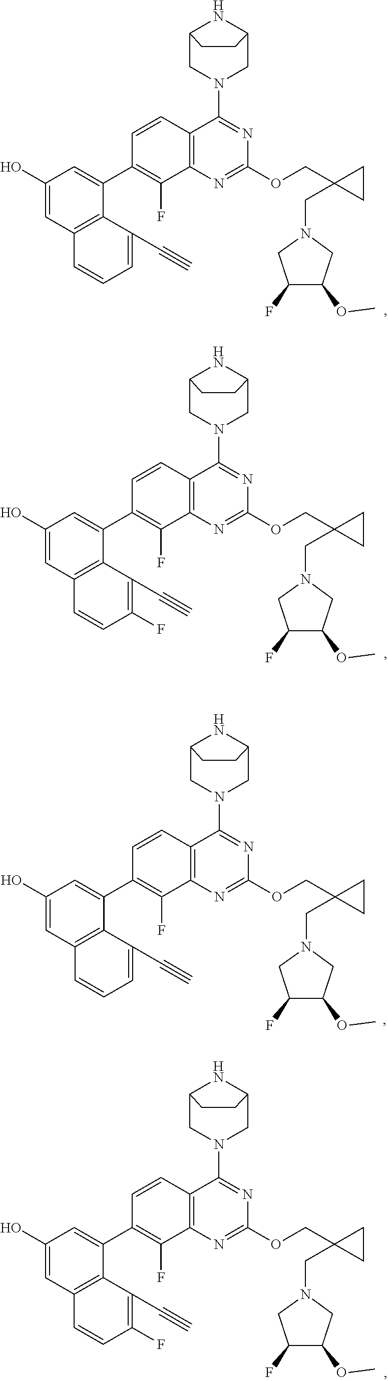 [SMILES: C#Cc1cccc2cc(O)cc(-c3ccc4c(N5CC6CCC(C5)N6)nc(OCC5(CN6C[C@H](F)[C@H](OC)C6)CC5)nc4c3F)c12 ; C#Cc1c(F)ccc2cc(O)cc(-c3ccc4c(N5CC6CCC(C5)N6)nc(OCC5(CN6C[C@H](F)[C@H](OC)C6)CC5)nc4c3F)c12 ; C#Cc1cccc2cc(O)cc(-c3ccc4c(N5CC6CCC(C5)N6)nc(OCC5(CN6C[C@H](F)[C@H](OC)C6)CC5)nc4c3F)c12 ; C#Cc1c(F)ccc2cc(O)cc(-c3ccc4c(N5CC6CCC(C5)N6)nc(OCC5(CN6C[C@H](F)[C@H](OC)C6)CC5)nc4c3F)c12]

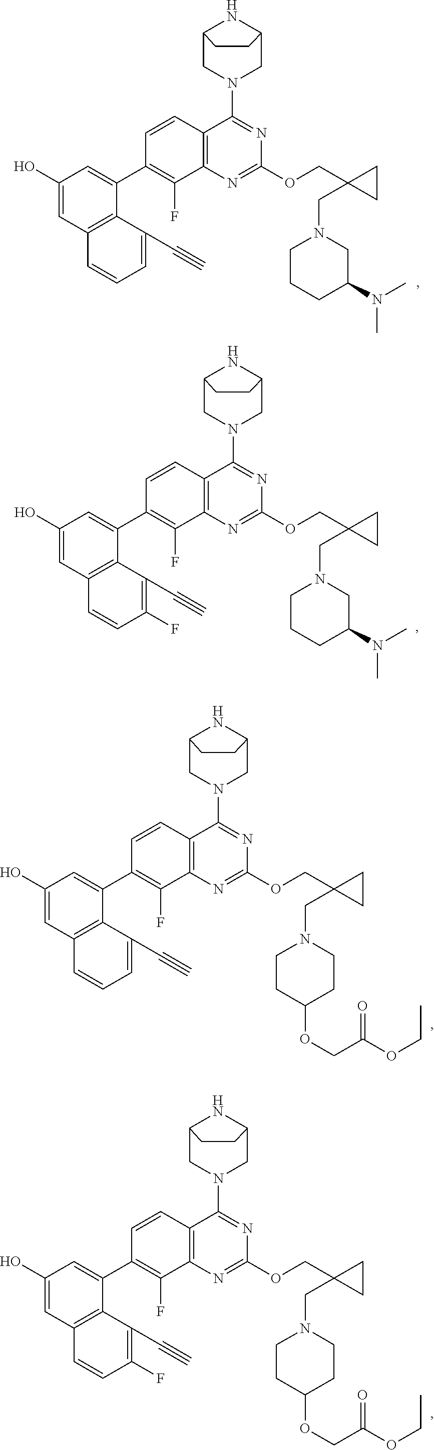 [SMILES: C#Cc1cccc2cc(O)cc(-c3ccc4c(N5CC6CCC(C5)N6)nc(OCC5(CN6CCC[C@H](N(C)C)C6)CC5)nc4c3F)c12 ; C#Cc1c(F)ccc2cc(O)cc(-c3ccc4c(N5CC6CCC(C5)N6)nc(OCC5(CN6CCC[C@H](N(C)C)C6)CC5)nc4c3F)c12 ; C#Cc1cccc2cc(O)cc(-c3ccc4c(N5CC6CCC(C5)N6)nc(OCC5(CN6CCC(OCC(=O)OCC)CC6)CC5)nc4c3F)c12 ; C#Cc1c(F)ccc2cc(O)cc(-c3ccc4c(N5CC6CCC(C5)N6)nc(OCC5(CN6CCC(OCC(=O)OCC)CC6)CC5)nc4c3F)c12]

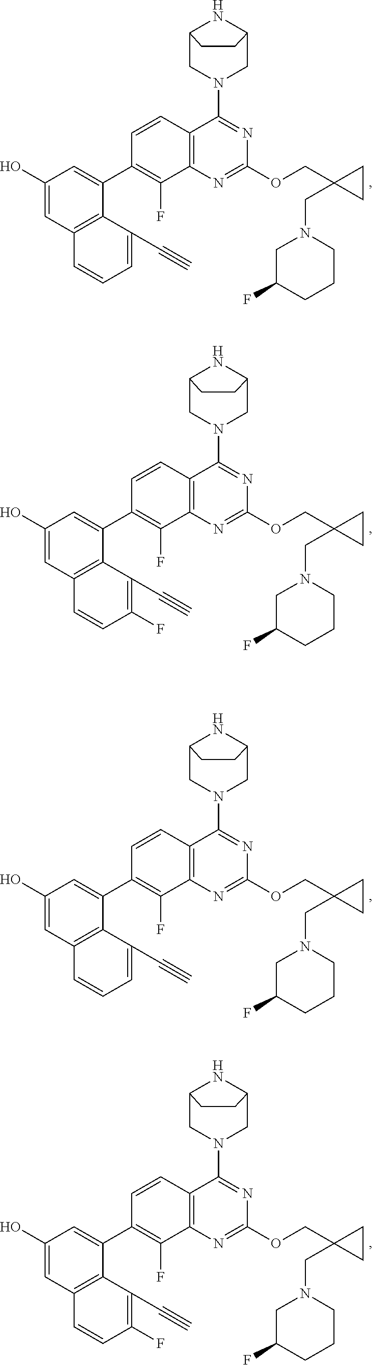 [SMILES: C#Cc1cccc2cc(O)cc(-c3ccc4c(N5CC6CCC(C5)N6)nc(OCC5(CN6CCC[C@@H](F)C6)CC5)nc4c3F)c12 ; C#Cc1c(F)ccc2cc(O)cc(-c3ccc4c(N5CC6CCC(C5)N6)nc(OCC5(CN6CCC[C@@H](F)C6)CC5)nc4c3F)c12 ; C#Cc1cccc2cc(O)cc(-c3ccc4c(N5CC6CCC(C5)N6)nc(OCC5(CN6CCC[C@@H](F)C6)CC5)nc4c3F)c12 ; C#Cc1c(F)ccc2cc(O)cc(-c3ccc4c(N5CC6CCC(C5)N6)nc(OCC5(CN6CCC[C@@H](F)C6)CC5)nc4c3F)c12]

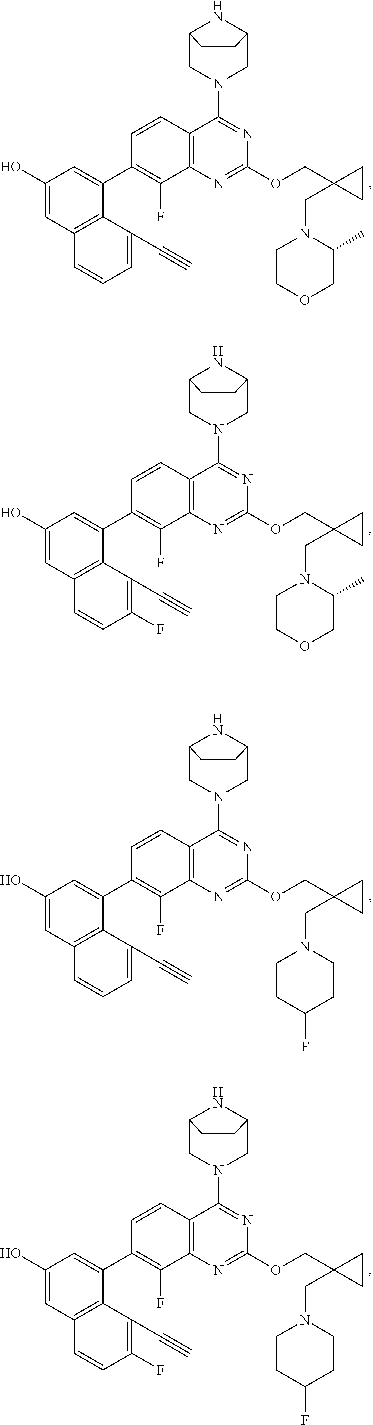 [SMILES: C#Cc1cccc2cc(O)cc(-c3ccc4c(N5CC6CCC(C5)N6)nc(OCC5(CN6CCOC[C@H]6C)CC5)nc4c3F)c12 ; C#Cc1c(F)ccc2cc(O)cc(-c3ccc4c(N5CC6CCC(C5)N6)nc(OCC5(CN6CCOC[C@H]6C)CC5)nc4c3F)c12 ; C#Cc1cccc2cc(O)cc(-c3ccc4c(N5CC6CCC(C5)N6)nc(OCC5(CN6CCC(F)CC6)CC5)nc4c3F)c12 ; C#Cc1c(F)ccc2cc(O)cc(-c3ccc4c(N5CC6CCC(C5)N6)nc(OCC5(CN6CCC(F)CC6)CC5)nc4c3F)c12]

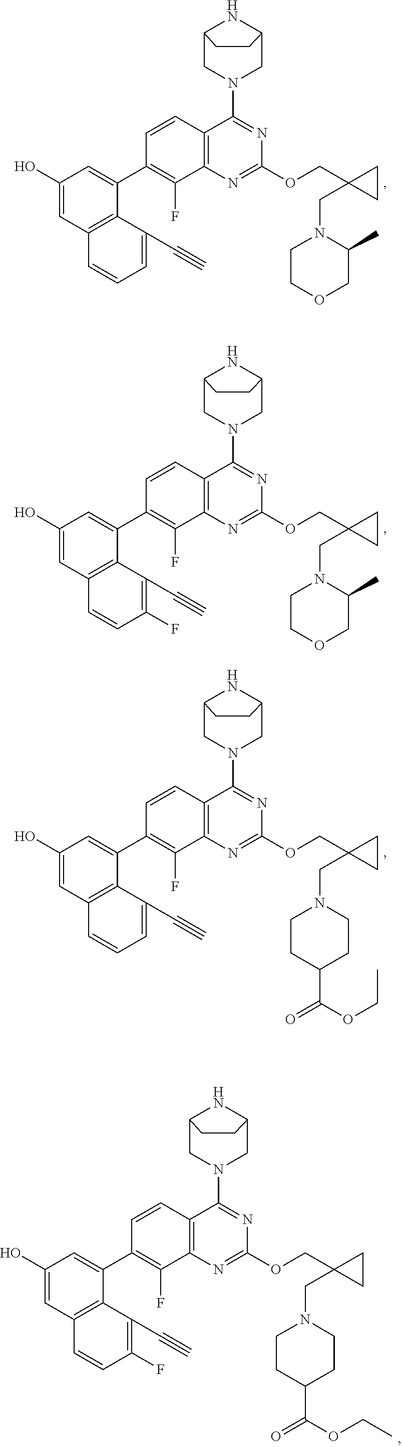 [SMILES: C#Cc1cccc2cc(O)cc(-c3ccc4c(N5CC6CCC(C5)N6)nc(OCC5(CN6CCOC[C@@H]6C)CC5)nc4c3F)c12 ; C#Cc1c(F)ccc2cc(O)cc(-c3ccc4c(N5CC6CCC(C5)N6)nc(OCC5(CN6CCOC[C@@H]6C)CC5)nc4c3F)c12 ; C#Cc1cccc2cc(O)cc(-c3ccc4c(N5CC6CCC(C5)N6)nc(OCC5(CN6CCC(C(=O)OCC)CC6)CC5)nc4c3F)c12 ; C#Cc1c(F)ccc2cc(O)cc(-c3ccc4c(N5CC6CCC(C5)N6)nc(OCC5(CN6CCC(C(=O)OCC)CC6)CC5)nc4c3F)c12]

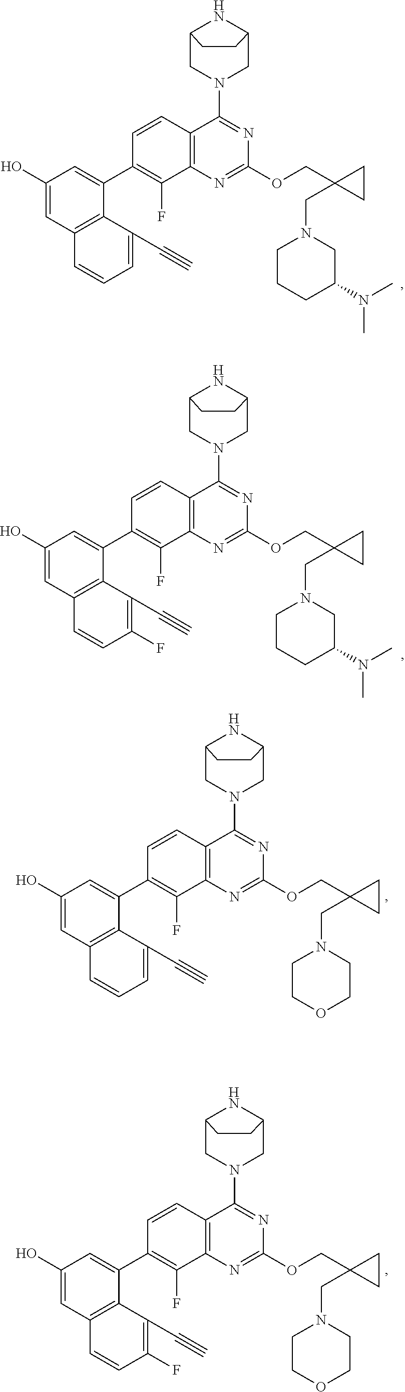 [SMILES: C#Cc1cccc2cc(O)cc(-c3ccc4c(N5CC6CCC(C5)N6)nc(OCC5(CN6CCC[C@@H](N(C)C)C6)CC5)nc4c3F)c12 ; C#Cc1c(F)ccc2cc(O)cc(-c3ccc4c(N5CC6CCC(C5)N6)nc(OCC5(CN6CCC[C@@H](N(C)C)C6)CC5)nc4c3F)c12 ; C#Cc1cccc2cc(O)cc(-c3ccc4c(N5CC6CCC(C5)N6)nc(OCC5(CN6CCOCC6)CC5)nc4c3F)c12 ; C#Cc1c(F)ccc2cc(O)cc(-c3ccc4c(N5CC6CCC(C5)N6)nc(OCC5(CN6CCOCC6)CC5)nc4c3F)c12]

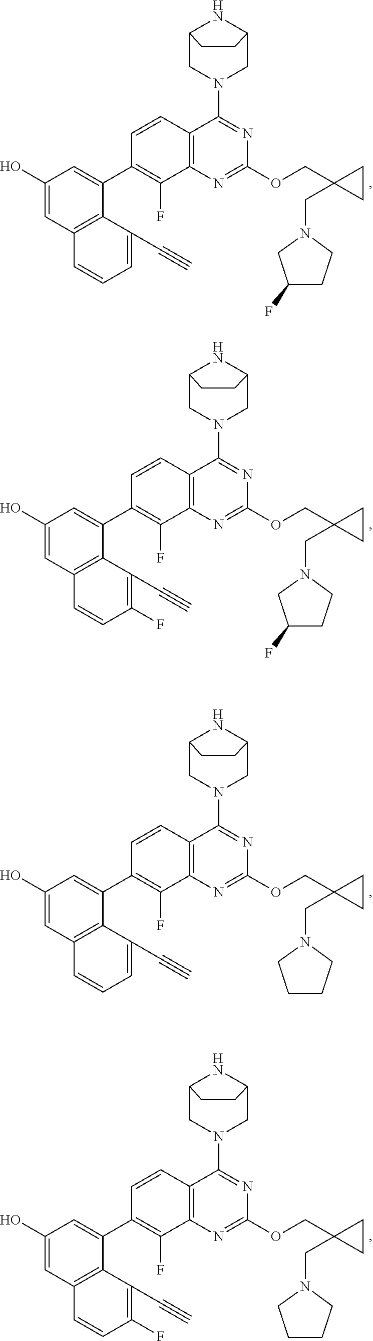 [SMILES: C#Cc1cccc2cc(O)cc(-c3ccc4c(N5CC6CCC(C5)N6)nc(OCC5(CN6CC[C@@H](F)C6)CC5)nc4c3F)c12 ; C#Cc1c(F)ccc2cc(O)cc(-c3ccc4c(N5CC6CCC(C5)N6)nc(OCC5(CN6CC[C@@H](F)C6)CC5)nc4c3F)c12 ; C#Cc1cccc2cc(O)cc(-c3ccc4c(N5CC6CCC(C5)N6)nc(OCC5(CN6CCCC6)CC5)nc4c3F)c12 ; C#Cc1c(F)ccc2cc(O)cc(-c3ccc4c(N5CC6CCC(C5)N6)nc(OCC5(CN6CCCC6)CC5)nc4c3F)c12]

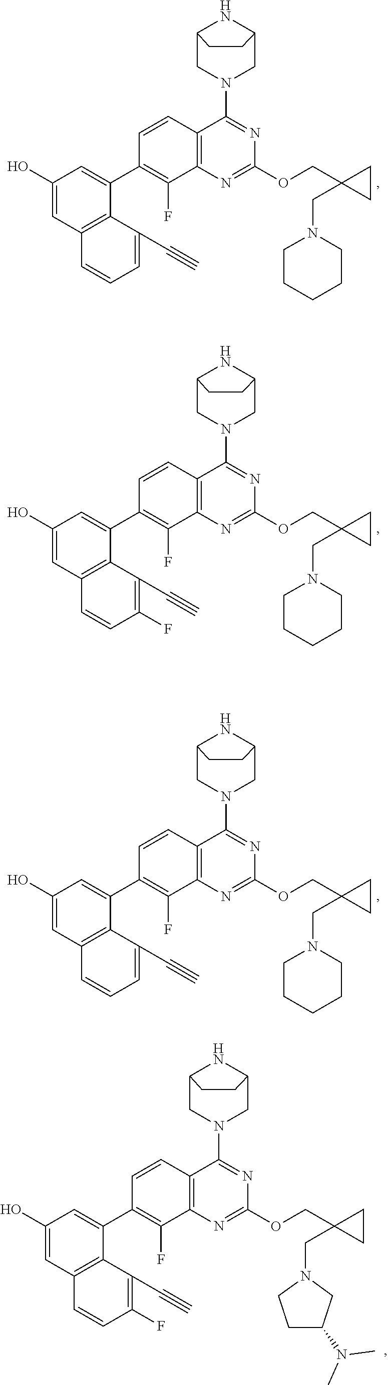 [SMILES: C#Cc1cccc2cc(O)cc(-c3ccc4c(N5CC6CCC(C5)N6)nc(OCC5(CN6CCCCC6)CC5)nc4c3F)c12 ; C#Cc1c(F)ccc2cc(O)cc(-c3ccc4c(N5CC6CCC(C5)N6)nc(OCC5(CN6CCCCC6)CC5)nc4c3F)c12 ; C#Cc1cccc2cc(O)cc(-c3ccc4c(N5CC6CCC(C5)N6)nc(OCC5(CN6CCCCC6)CC5)nc4c3F)c12 ; C#Cc1c(F)ccc2cc(O)cc(-c3ccc4c(N5CC6CCC(C5)N6)nc(OCC5(CN6CC[C@@H](N(C)C)C6)CC5)nc4c3F)c12]

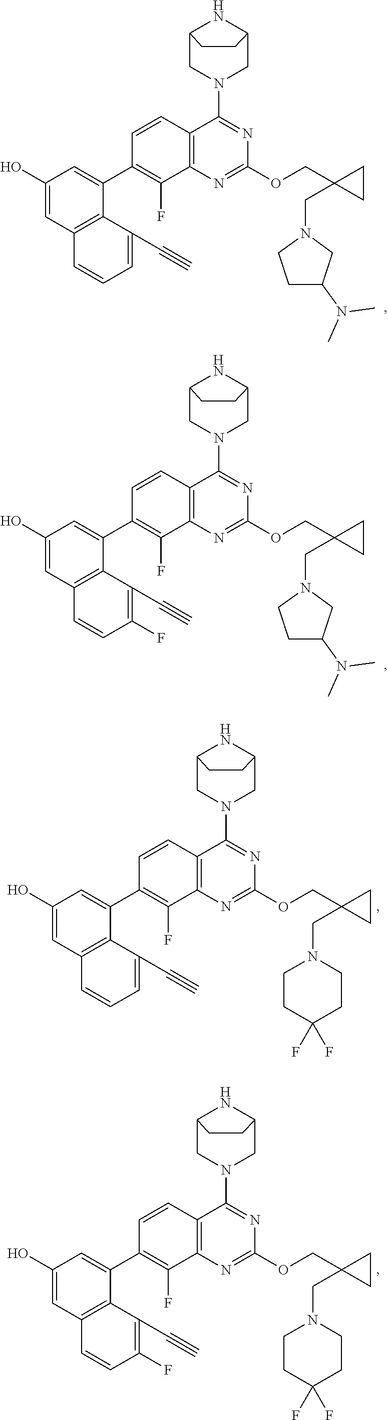 [SMILES: C#Cc1cccc2cc(O)cc(-c3ccc4c(N5CC6CCC(C5)N6)nc(OCC5(CN6CCC(N(C)C)C6)CC5)nc4c3F)c12 ; C#Cc1c(F)ccc2cc(O)cc(-c3ccc4c(N5CC6CCC(C5)N6)nc(OCC5(CN6CCC(N(C)C)C6)CC5)nc4c3F)c12 ; C#Cc1cccc2cc(O)cc(-c3ccc4c(N5CC6CCC(C5)N6)nc(OCC5(CN6CCC(F)(F)CC6)CC5)nc4c3F)c12 ; C#Cc1c(F)ccc2cc(O)cc(-c3ccc4c(N5CC6CCC(C5)N6)nc(OCC5(CN6CCC(F)(F)CC6)CC5)nc4c3F)c12]

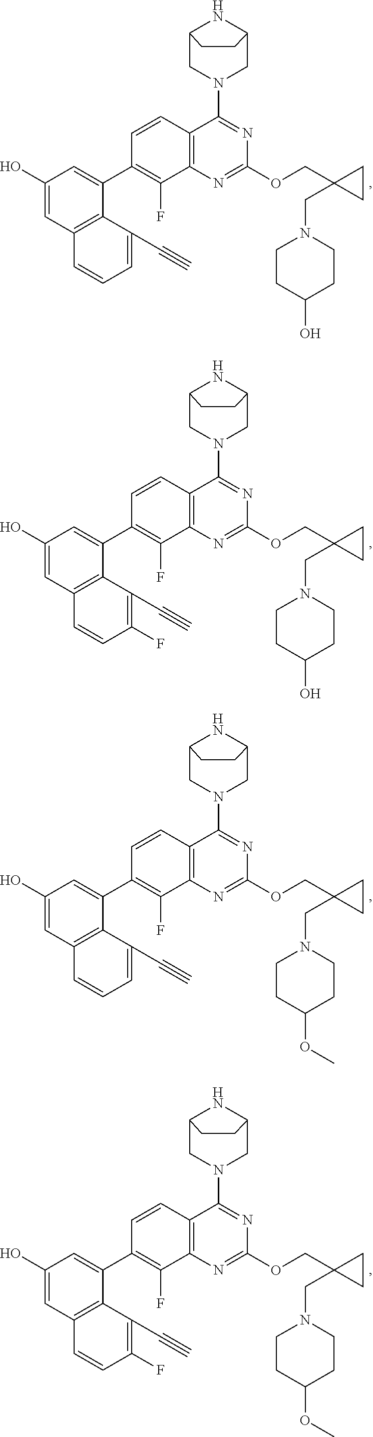 [SMILES: C#Cc1cccc2cc(O)cc(-c3ccc4c(N5CC6CCC(C5)N6)nc(OCC5(CN6CCC(O)CC6)CC5)nc4c3F)c12 ; C#Cc1c(F)ccc2cc(O)cc(-c3ccc4c(N5CC6CCC(C5)N6)nc(OCC5(CN6CCC(O)CC6)CC5)nc4c3F)c12 ; C#Cc1cccc2cc(O)cc(-c3ccc4c(N5CC6CCC(C5)N6)nc(OCC5(CN6CCC(OC)CC6)CC5)nc4c3F)c12 ; C#Cc1c(F)ccc2cc(O)cc(-c3ccc4c(N5CC6CCC(C5)N6)nc(OCC5(CN6CCC(OC)CC6)CC5)nc4c3F)c12]

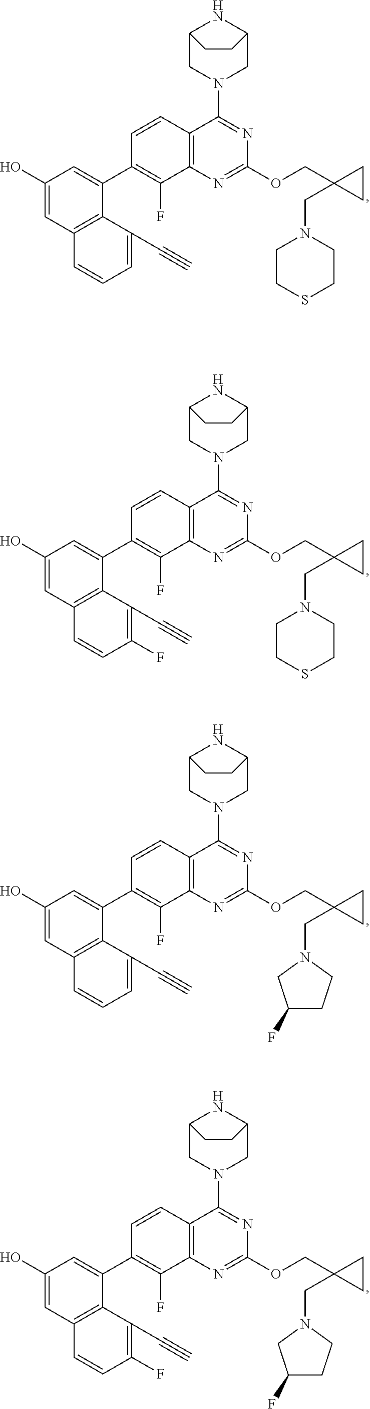 [SMILES: C#Cc1cccc2cc(O)cc(-c3ccc4c(N5CC6CCC(C5)N6)nc(OCC5(CN6CCSCC6)CC5)nc4c3F)c12 ; C#Cc1c(F)ccc2cc(O)cc(-c3ccc4c(N5CC6CCC(C5)N6)nc(OCC5(CN6CCSCC6)CC5)nc4c3F)c12 ; C#Cc1cccc2cc(O)cc(-c3ccc4c(N5CC6CCC(C5)N6)nc(OCC5(CN6CC[C@@H](F)C6)CC5)nc4c3F)c12 ; C#Cc1c(F)ccc2cc(O)cc(-c3ccc4c(N5CC6CCC(C5)N6)nc(OCC5(CN6CC[C@@H](F)C6)CC5)nc4c3F)c12]

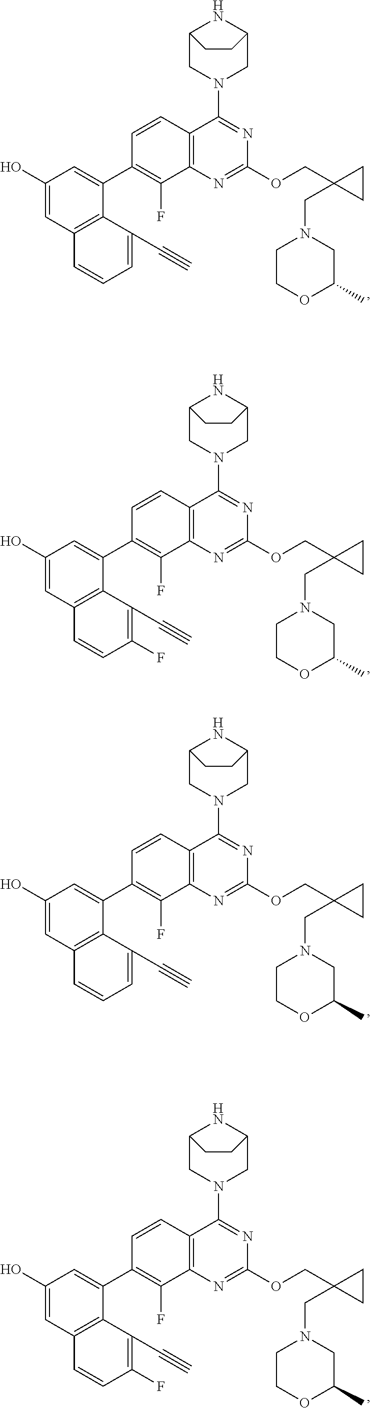 [SMILES: C#Cc1cccc2cc(O)cc(-c3ccc4c(N5CC6CCC(C5)N6)nc(OCC5(CN6CCO[C@@H](C)C6)CC5)nc4c3F)c12 ; C#Cc1c(F)ccc2cc(O)cc(-c3ccc4c(N5CC6CCC(C5)N6)nc(OCC5(CN6CCO[C@@H](C)C6)CC5)nc4c3F)c12 ; C#Cc1cccc2cc(O)cc(-c3ccc4c(N5CC6CCC(C5)N6)nc(OCC5(CN6CCO[C@H](C)C6)CC5)nc4c3F)c12 ; C#Cc1c(F)ccc2cc(O)cc(-c3ccc4c(N5CC6CCC(C5)N6)nc(OCC5(CN6CCO[C@H](C)C6)CC5)nc4c3F)c12]

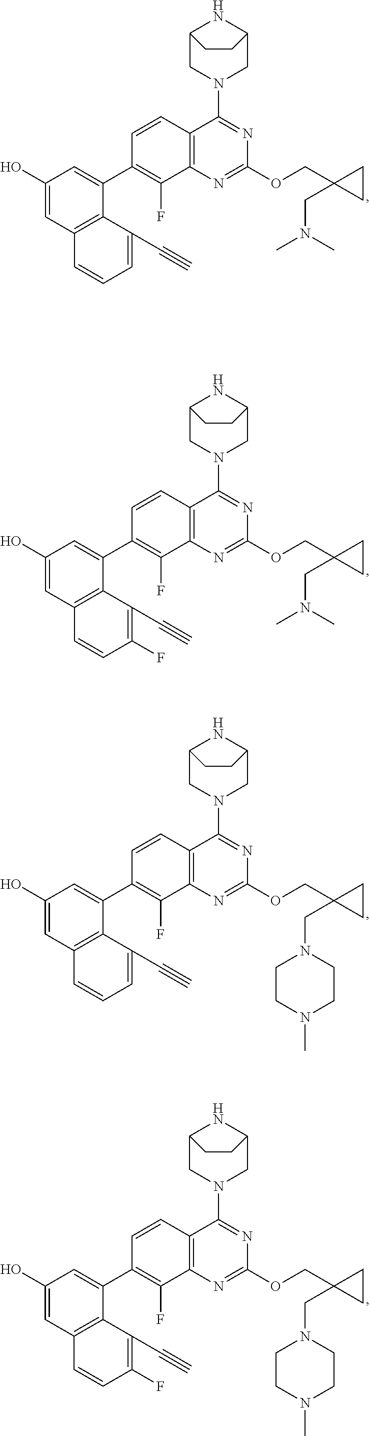 [SMILES: C#Cc1cccc2cc(O)cc(-c3ccc4c(N5CC6CCC(C5)N6)nc(OCC5(CN(C)C)CC5)nc4c3F)c12 ; C#Cc1c(F)ccc2cc(O)cc(-c3ccc4c(N5CC6CCC(C5)N6)nc(OCC5(CN(C)C)CC5)nc4c3F)c12 ; C#Cc1cccc2cc(O)cc(-c3ccc4c(N5CC6CCC(C5)N6)nc(OCC5(CN6CCN(C)CC6)CC5)nc4c3F)c12 ; C#Cc1c(F)ccc2cc(O)cc(-c3ccc4c(N5CC6CCC(C5)N6)nc(OCC5(CN6CCN(C)CC6)CC5)nc4c3F)c12]

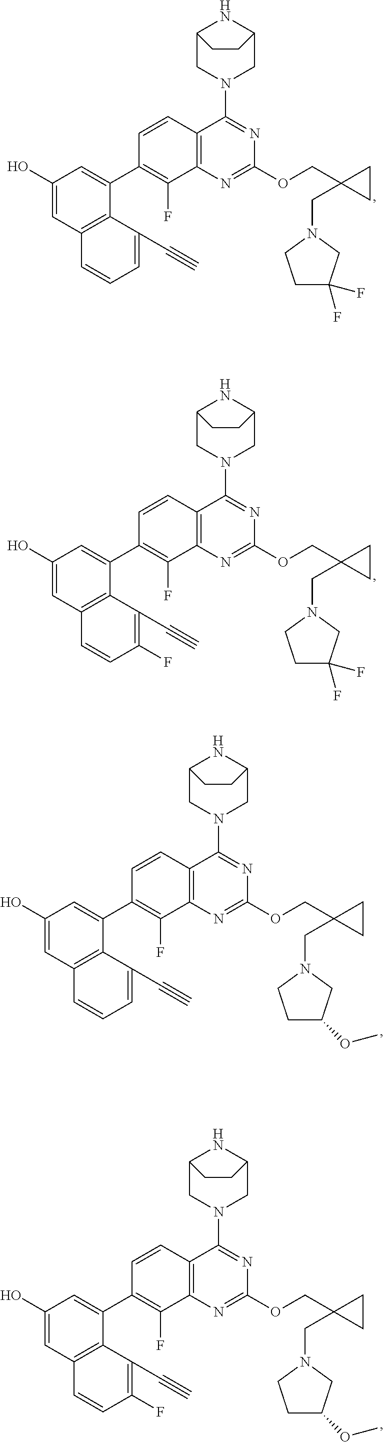 [SMILES: C#Cc1cccc2cc(O)cc(-c3ccc4c(N5CC6CCC(C5)N6)nc(OCC5(CN6CCC(F)(F)C6)CC5)nc4c3F)c12 ; C#Cc1c(F)ccc2cc(O)cc(-c3ccc4c(N5CC6CCC(C5)N6)nc(OCC5(CN6CCC(F)(F)C6)CC5)nc4c3F)c12 ; C#Cc1cccc2cc(O)cc(-c3ccc4c(N5CC6CCC(C5)N6)nc(OCC5(CN6CC[C@@H](OC)C6)CC5)nc4c3F)c12 ; C#Cc1c(F)ccc2cc(O)cc(-c3ccc4c(N5CC6CCC(C5)N6)nc(OCC5(CN6CC[C@@H](OC)C6)CC5)nc4c3F)c12]

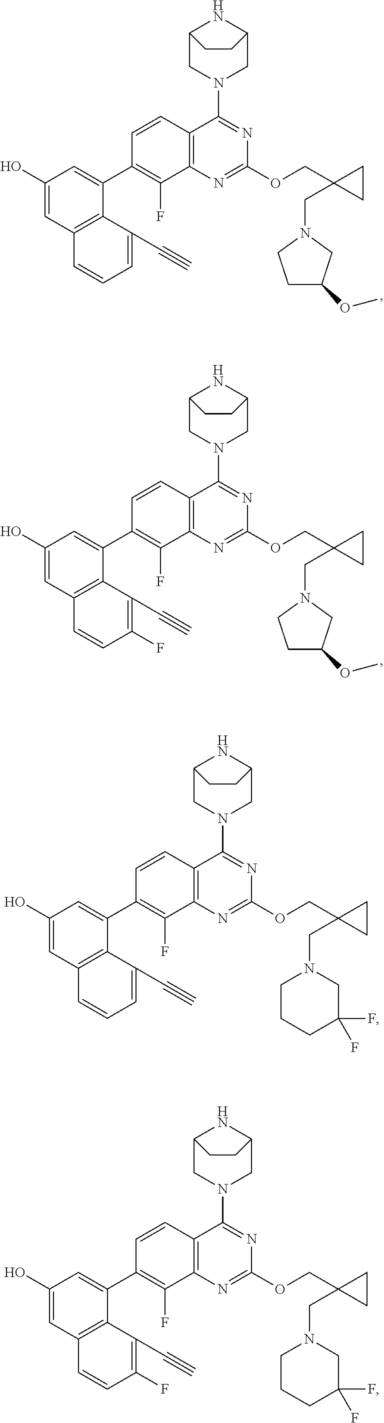 [SMILES: C#Cc1cccc2cc(O)cc(-c3ccc4c(N5CC6CCC(C5)N6)nc(OCC5(CN6CC[C@H](OC)C6)CC5)nc4c3F)c12 ; C#Cc1c(F)ccc2cc(O)cc(-c3ccc4c(N5CC6CCC(C5)N6)nc(OCC5(CN6CC[C@H](OC)C6)CC5)nc4c3F)c12 ; C#Cc1cccc2cc(O)cc(-c3ccc4c(N5CC6CCC(C5)N6)nc(OCC5(CN6CCCC(F)(F)C6)CC5)nc4c3F)c12 ; C#Cc1c(F)ccc2cc(O)cc(-c3ccc4c(N5CC6CCC(C5)N6)nc(OCC5(CN6CCCC(F)(F)C6)CC5)nc4c3F)c12]

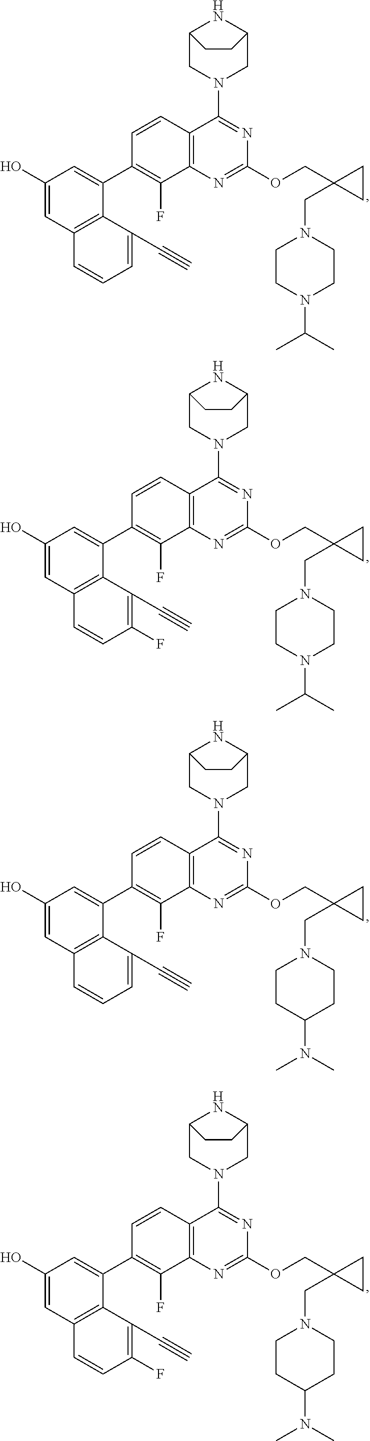 [SMILES: C#Cc1cccc2cc(O)cc(-c3ccc4c(N5CC6CCC(C5)N6)nc(OCC5(CN6CCN(C(C)C)CC6)CC5)nc4c3F)c12 ; C#Cc1c(F)ccc2cc(O)cc(-c3ccc4c(N5CC6CCC(C5)N6)nc(OCC5(CN6CCN(C(C)C)CC6)CC5)nc4c3F)c12 ; C#Cc1cccc2cc(O)cc(-c3ccc4c(N5CC6CCC(C5)N6)nc(OCC5(CN6CCC(N(C)C)CC6)CC5)nc4c3F)c12 ; C#Cc1c(F)ccc2cc(O)cc(-c3ccc4c(N5CC6CCC(C5)N6)nc(OCC5(CN6CCC(N(C)C)CC6)CC5)nc4c3F)c12]

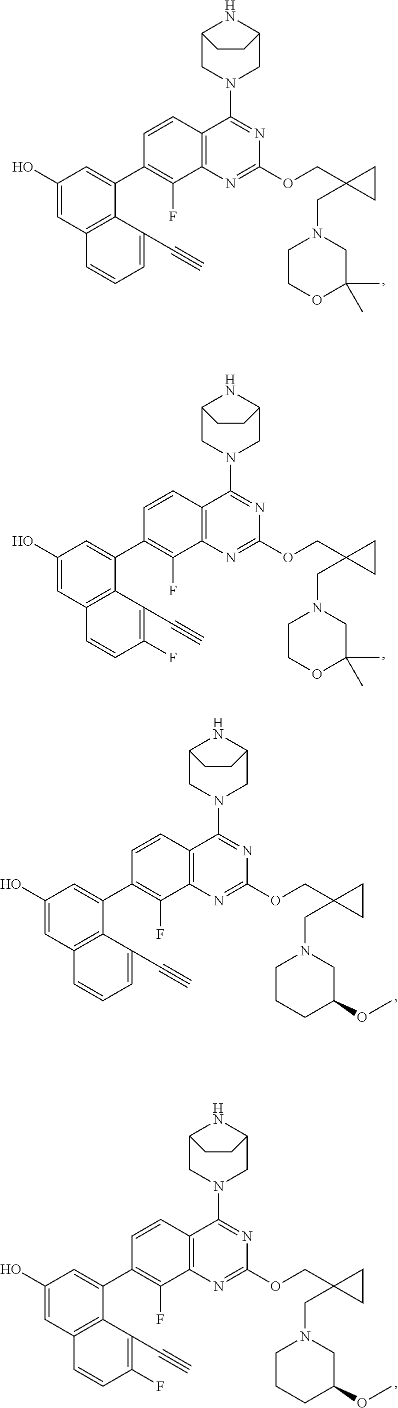 [SMILES: C#Cc1cccc2cc(O)cc(-c3ccc4c(N5CC6CCC(C5)N6)nc(OCC5(CN6CCOC(C)(C)C6)CC5)nc4c3F)c12 ; C#Cc1c(F)ccc2cc(O)cc(-c3ccc4c(N5CC6CCC(C5)N6)nc(OCC5(CN6CCOC(C)(C)C6)CC5)nc4c3F)c12 ; C#Cc1cccc2cc(O)cc(-c3ccc4c(N5CC6CCC(C5)N6)nc(OCC5(CN6CCC[C@H](OC)C6)CC5)nc4c3F)c12 ; C#Cc1c(F)ccc2cc(O)cc(-c3ccc4c(N5CC6CCC(C5)N6)nc(OCC5(CN6CCC[C@H](OC)C6)CC5)nc4c3F)c12]

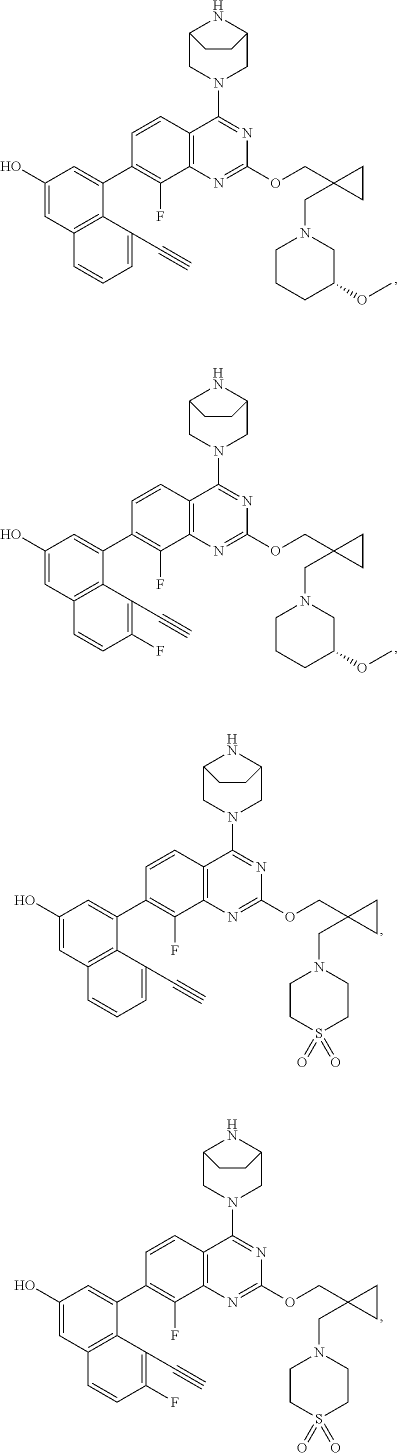 [SMILES: C#Cc1cccc2cc(O)cc(-c3ccc4c(N5CC6CCC(C5)N6)nc(OCC5(CN6CCC[C@@H](OC)C6)CC5)nc4c3F)c12 ; C#Cc1c(F)ccc2cc(O)cc(-c3ccc4c(N5CC6CCC(C5)N6)nc(OCC5(CN6CCC[C@@H](OC)C6)CC5)nc4c3F)c12 ; C#Cc1cccc2cc(O)cc(-c3ccc4c(N5CC6CCC(C5)N6)nc(OCC5(CN6CCS(=O)(=O)CC6)CC5)nc4c3F)c12 ; C#Cc1c(F)ccc2cc(O)cc(-c3ccc4c(N5CC6CCC(C5)N6)nc(OCC5(CN6CCS(=O)(=O)CC6)CC5)nc4c3F)c12]

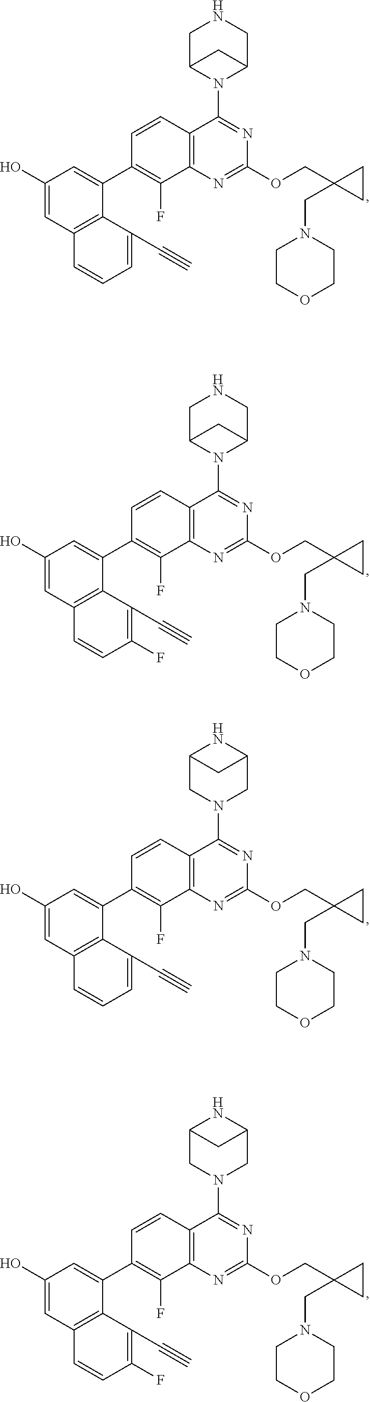 [SMILES: C#Cc1cccc2cc(O)cc(-c3ccc4c(N5C6CNCC5C6)nc(OCC5(CN6CCOCC6)CC5)nc4c3F)c12 ; C#Cc1c(F)ccc2cc(O)cc(-c3ccc4c(N5C6CNCC5C6)nc(OCC5(CN6CCOCC6)CC5)nc4c3F)c12 ; C#Cc1cccc2cc(O)cc(-c3ccc4c(N5CC6CC(C5)N6)nc(OCC5(CN6CCOCC6)CC5)nc4c3F)c12 ; C#Cc1c(F)ccc2cc(O)cc(-c3ccc4c(N5CC6CC(C5)N6)nc(OCC5(CN6CCOCC6)CC5)nc4c3F)c12]

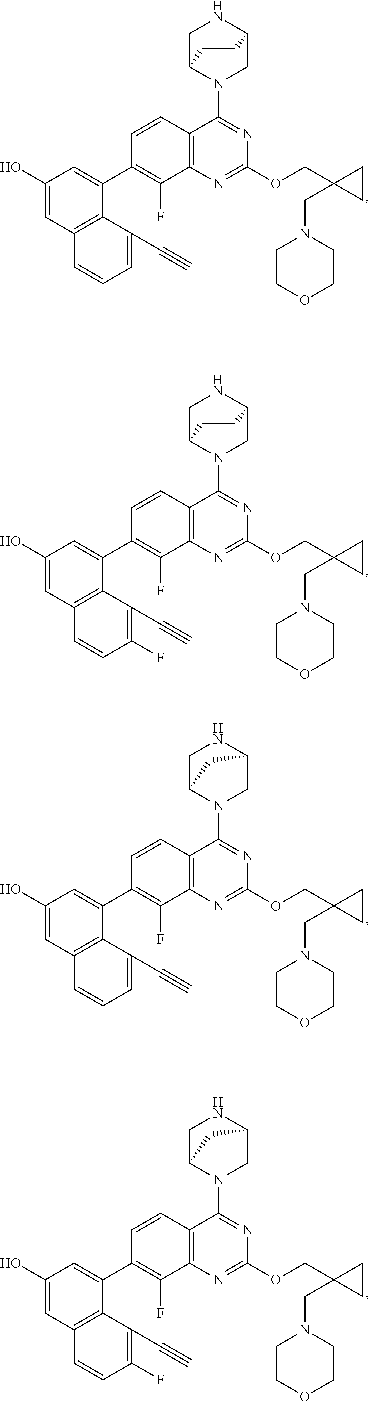 [SMILES: C#Cc1cccc2cc(O)cc(-c3ccc4c(N5C[C@H]6CC[C@@H]5CN6)nc(OCC5(CN6CCOCC6)CC5)nc4c3F)c12 ; C#Cc1c(F)ccc2cc(O)cc(-c3ccc4c(N5C[C@H]6CC[C@@H]5CN6)nc(OCC5(CN6CCOCC6)CC5)nc4c3F)c12 ; C#Cc1cccc2cc(O)cc(-c3ccc4c(N5C[C@H]6C[C@@H]5CN6)nc(OCC5(CN6CCOCC6)CC5)nc4c3F)c12 ; C#Cc1c(F)ccc2cc(O)cc(-c3ccc4c(N5C[C@H]6C[C@@H]5CN6)nc(OCC5(CN6CCOCC6)CC5)nc4c3F)c12]

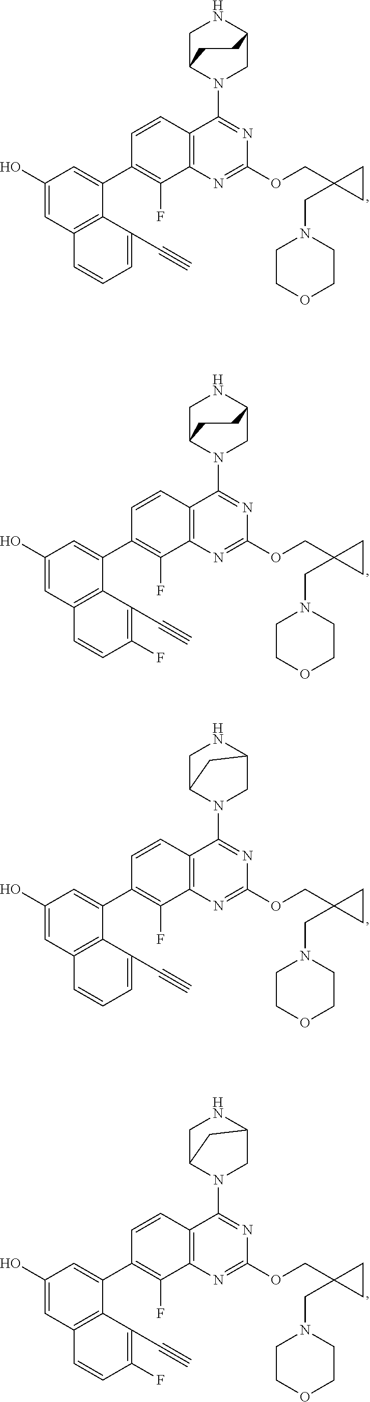 [SMILES: C#Cc1cccc2cc(O)cc(-c3ccc4c(N5C[C@@H]6CC[C@H]5CN6)nc(OCC5(CN6CCOCC6)CC5)nc4c3F)c12 ; C#Cc1c(F)ccc2cc(O)cc(-c3ccc4c(N5C[C@@H]6CC[C@H]5CN6)nc(OCC5(CN6CCOCC6)CC5)nc4c3F)c12 ; C#Cc1cccc2cc(O)cc(-c3ccc4c(N5CC6CC5CN6)nc(OCC5(CN6CCOCC6)CC5)nc4c3F)c12 ; C#Cc1c(F)ccc2cc(O)cc(-c3ccc4c(N5CC6CC5CN6)nc(OCC5(CN6CCOCC6)CC5)nc4c3F)c12]

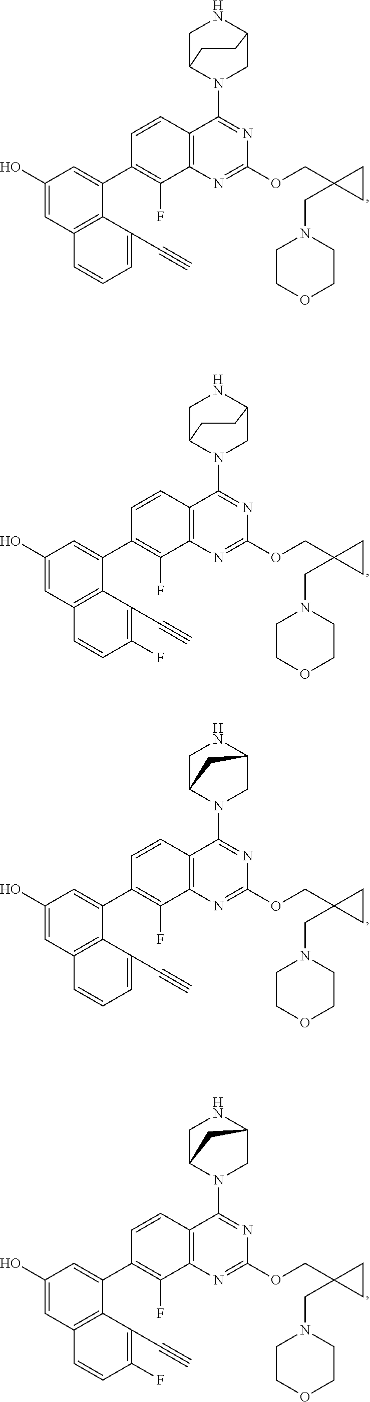 [SMILES: C#Cc1cccc2cc(O)cc(-c3ccc4c(N5CC6CCC5CN6)nc(OCC5(CN6CCOCC6)CC5)nc4c3F)c12 ; C#Cc1c(F)ccc2cc(O)cc(-c3ccc4c(N5CC6CCC5CN6)nc(OCC5(CN6CCOCC6)CC5)nc4c3F)c12 ; C#Cc1cccc2cc(O)cc(-c3ccc4c(N5C[C@@H]6C[C@H]5CN6)nc(OCC5(CN6CCOCC6)CC5)nc4c3F)c12 ; C#Cc1c(F)ccc2cc(O)cc(-c3ccc4c(N5C[C@@H]6C[C@H]5CN6)nc(OCC5(CN6CCOCC6)CC5)nc4c3F)c12]

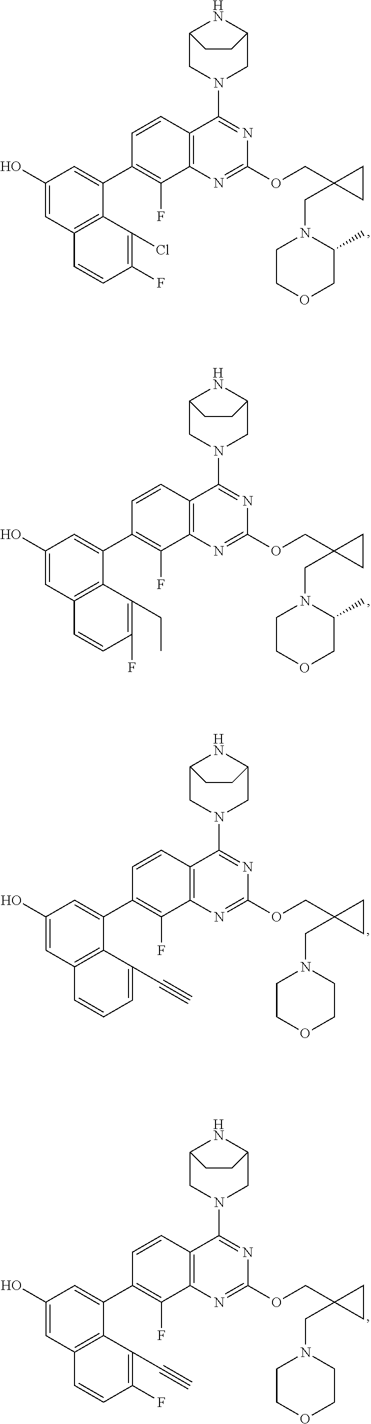 [SMILES: C[C@@H]1COCCN1CC1(COc2nc(N3CC4CCC(C3)N4)c3ccc(-c4cc(O)cc5ccc(F)c(Cl)c45)c(F)c3n2)CC1 ; CCc1c(F)ccc2cc(O)cc(-c3ccc4c(N5CC6CCC(C5)N6)nc(OCC5(CN6CCOC[C@H]6C)CC5)nc4c3F)c12 ; C#Cc1cccc2cc(O)cc(-c3ccc4c(N5CC6CCC(C5)N6)nc(OCC5(CN6CCOCC6)CC5)nc4c3F)c12 ; C#Cc1c(F)ccc2cc(O)cc(-c3ccc4c(N5CC6CCC(C5)N6)nc(OCC5(CN6CCOCC6)CC5)nc4c3F)c12]

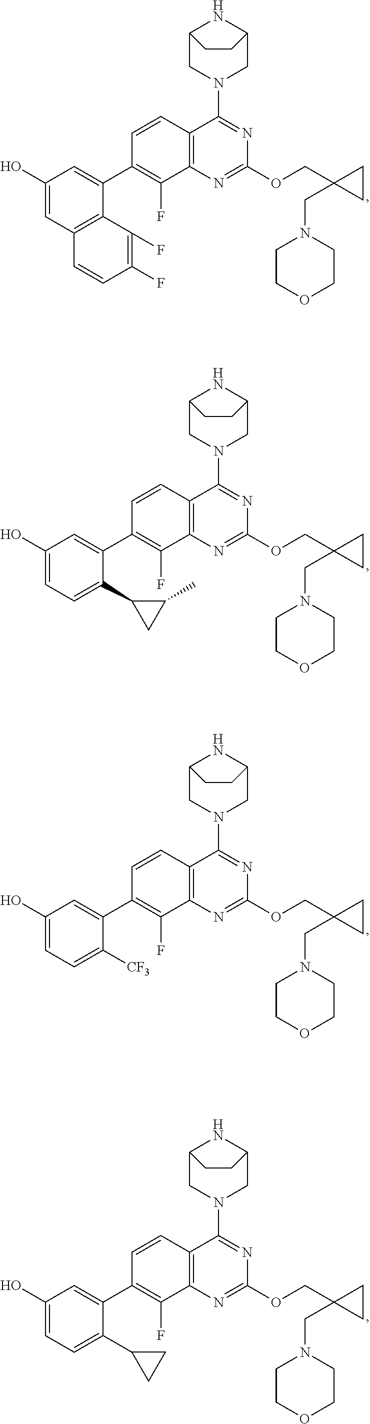 [SMILES: Oc1cc(-c2ccc3c(N4CC5CCC(C4)N5)nc(OCC4(CN5CCOCC5)CC4)nc3c2F)c2c(F)c(F)ccc2c1 ; C[C@@H]1C[C@H]1c1ccc(O)cc1-c1ccc2c(N3CC4CCC(C3)N4)nc(OCC3(CN4CCOCC4)CC3)nc2c1F ; Oc1ccc(C(F)(F)F)c(-c2ccc3c(N4CC5CCC(C4)N5)nc(OCC4(CN5CCOCC5)CC4)nc3c2F)c1 ; Oc1ccc(C2CC2)c(-c2ccc3c(N4CC5CCC(C4)N5)nc(OCC4(CN5CCOCC5)CC4)nc3c2F)c1]

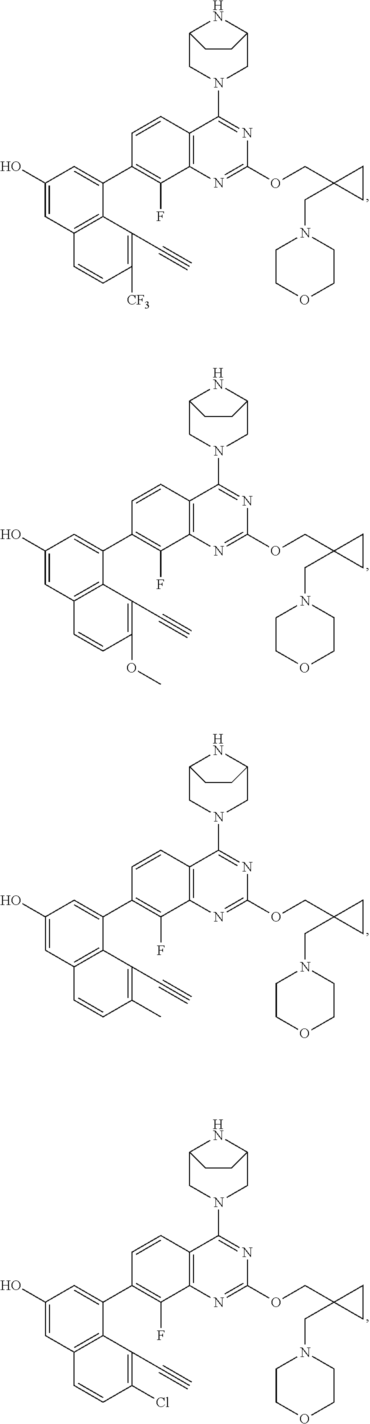 [SMILES: C#Cc1c(C(F)(F)F)ccc2cc(O)cc(-c3ccc4c(N5CC6CCC(C5)N6)nc(OCC5(CN6CCOCC6)CC5)nc4c3F)c12 ; C#Cc1c(OC)ccc2cc(O)cc(-c3ccc4c(N5CC6CCC(C5)N6)nc(OCC5(CN6CCOCC6)CC5)nc4c3F)c12 ; C#Cc1c(C)ccc2cc(O)cc(-c3ccc4c(N5CC6CCC(C5)N6)nc(OCC5(CN6CCOCC6)CC5)nc4c3F)c12 ; C#Cc1c(Cl)ccc2cc(O)cc(-c3ccc4c(N5CC6CCC(C5)N6)nc(OCC5(CN6CCOCC6)CC5)nc4c3F)c12]

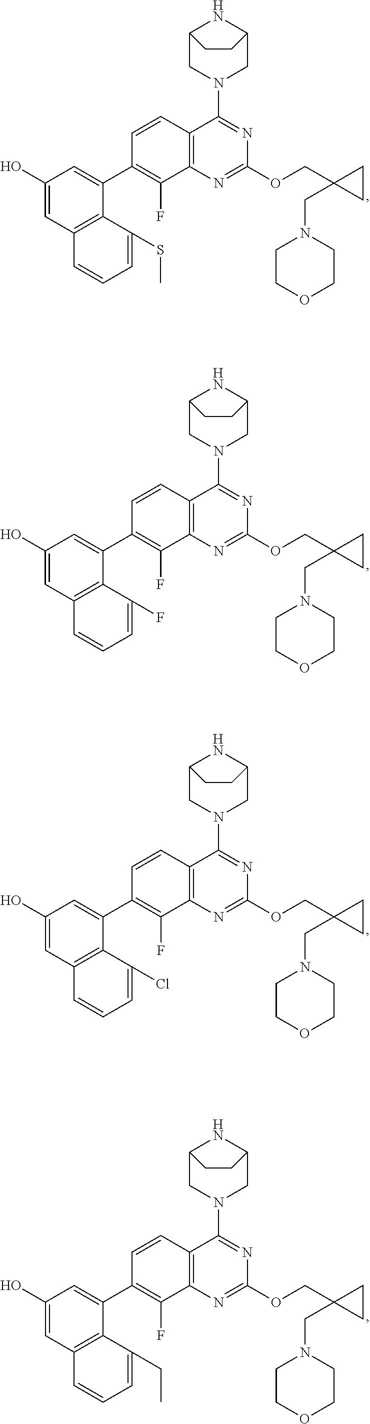 [SMILES: CSc1cccc2cc(O)cc(-c3ccc4c(N5CC6CCC(C5)N6)nc(OCC5(CN6CCOCC6)CC5)nc4c3F)c12 ; Oc1cc(-c2ccc3c(N4CC5CCC(C4)N5)nc(OCC4(CN5CCOCC5)CC4)nc3c2F)c2c(F)cccc2c1 ; Oc1cc(-c2ccc3c(N4CC5CCC(C4)N5)nc(OCC4(CN5CCOCC5)CC4)nc3c2F)c2c(Cl)cccc2c1 ; CCc1cccc2cc(O)cc(-c3ccc4c(N5CC6CCC(C5)N6)nc(OCC5(CN6CCOCC6)CC5)nc4c3F)c12]

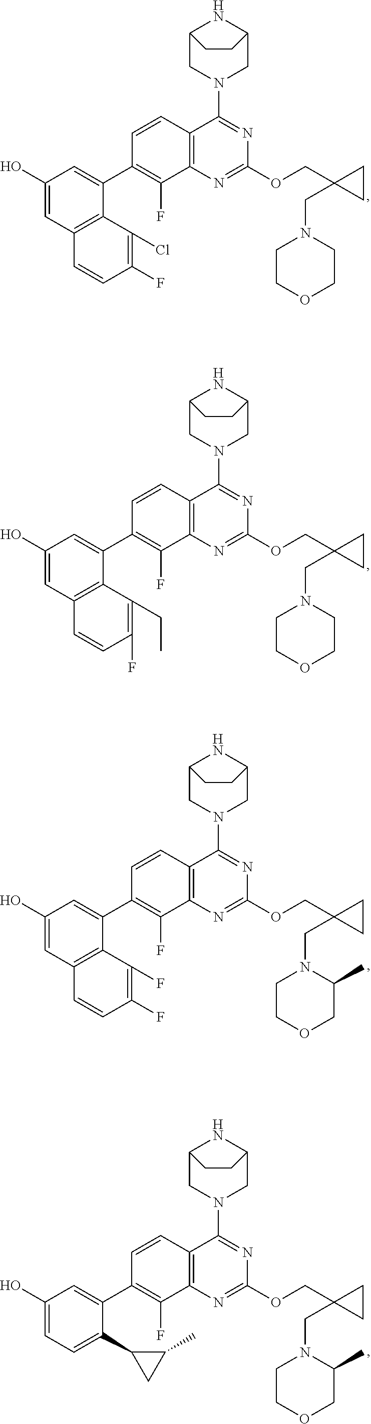 [SMILES: Oc1cc(-c2ccc3c(N4CC5CCC(C4)N5)nc(OCC4(CN5CCOCC5)CC4)nc3c2F)c2c(Cl)c(F)ccc2c1 ; CCc1c(F)ccc2cc(O)cc(-c3ccc4c(N5CC6CCC(C5)N6)nc(OCC5(CN6CCOCC6)CC5)nc4c3F)c12 ; C[C@H]1COCCN1CC1(COc2nc(N3CC4CCC(C3)N4)c3ccc(-c4cc(O)cc5ccc(F)c(F)c45)c(F)c3n2)CC1 ; C[C@@H]1C[C@H]1c1ccc(O)cc1-c1ccc2c(N3CC4CCC(C3)N4)nc(OCC3(CN4CCOC[C@@H]4C)CC3)nc2c1F]

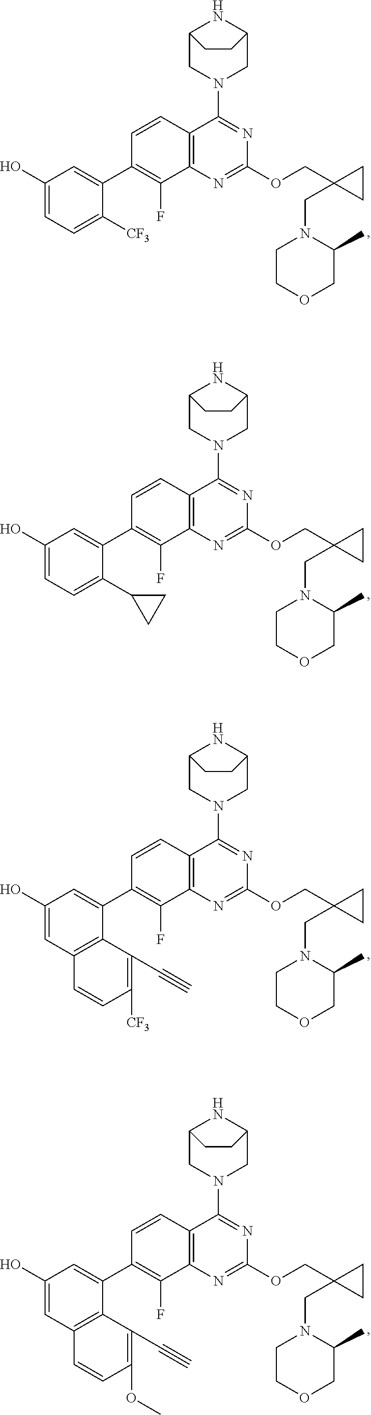 [SMILES: C[C@H]1COCCN1CC1(COc2nc(N3CC4CCC(C3)N4)c3ccc(-c4cc(O)ccc4C(F)(F)F)c(F)c3n2)CC1 ; C[C@H]1COCCN1CC1(COc2nc(N3CC4CCC(C3)N4)c3ccc(-c4cc(O)ccc4C4CC4)c(F)c3n2)CC1 ; C#Cc1c(C(F)(F)F)ccc2cc(O)cc(-c3ccc4c(N5CC6CCC(C5)N6)nc(OCC5(CN6CCOC[C@@H]6C)CC5)nc4c3F)c12 ; C#Cc1c(OC)ccc2cc(O)cc(-c3ccc4c(N5CC6CCC(C5)N6)nc(OCC5(CN6CCOC[C@@H]6C)CC5)nc4c3F)c12]

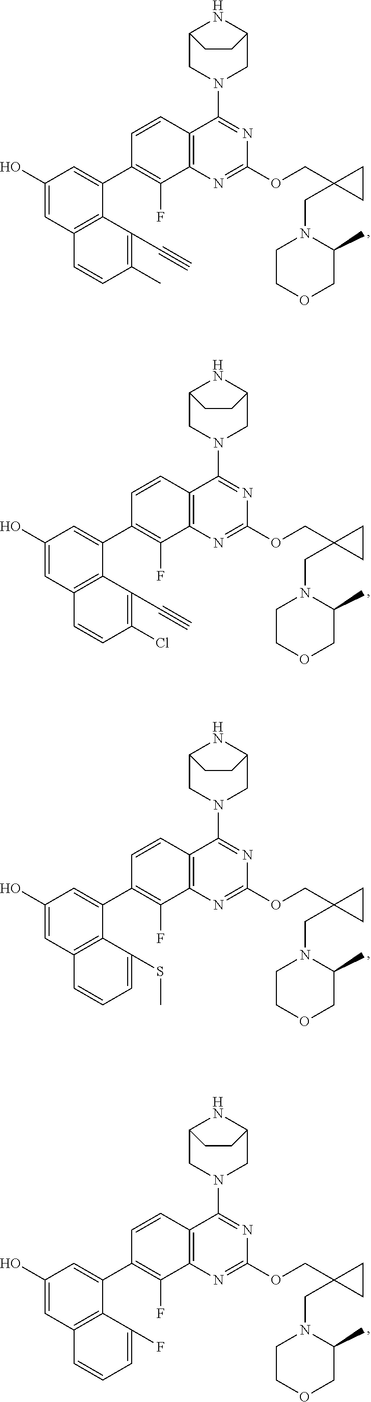 [SMILES: C#Cc1c(C)ccc2cc(O)cc(-c3ccc4c(N5CC6CCC(C5)N6)nc(OCC5(CN6CCOC[C@@H]6C)CC5)nc4c3F)c12 ; C#Cc1c(Cl)ccc2cc(O)cc(-c3ccc4c(N5CC6CCC(C5)N6)nc(OCC5(CN6CCOC[C@@H]6C)CC5)nc4c3F)c12 ; CSc1cccc2cc(O)cc(-c3ccc4c(N5CC6CCC(C5)N6)nc(OCC5(CN6CCOC[C@@H]6C)CC5)nc4c3F)c12 ; C[C@H]1COCCN1CC1(COc2nc(N3CC4CCC(C3)N4)c3ccc(-c4cc(O)cc5cccc(F)c45)c(F)c3n2)CC1]

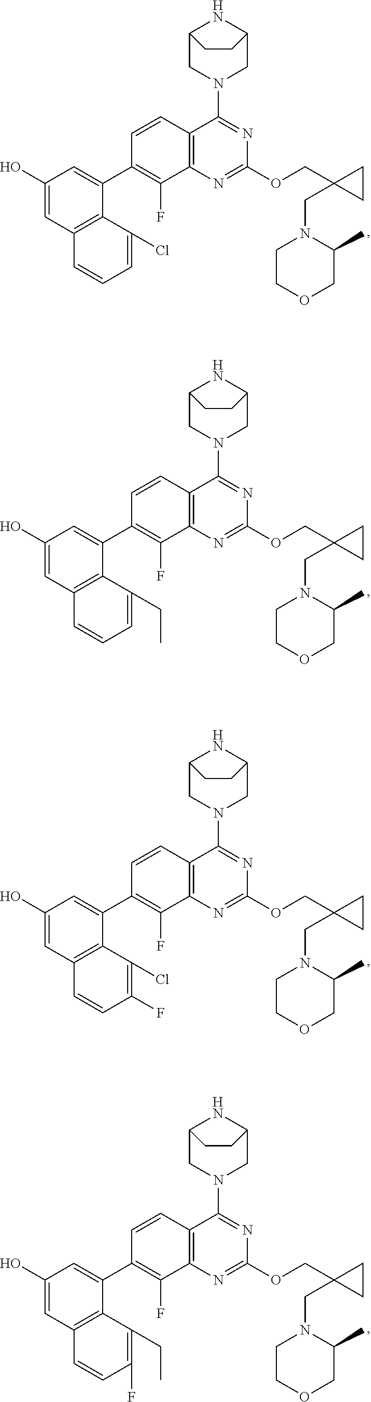 [SMILES: C[C@H]1COCCN1CC1(COc2nc(N3CC4CCC(C3)N4)c3ccc(-c4cc(O)cc5cccc(Cl)c45)c(F)c3n2)CC1 ; CCc1cccc2cc(O)cc(-c3ccc4c(N5CC6CCC(C5)N6)nc(OCC5(CN6CCOC[C@@H]6C)CC5)nc4c3F)c12 ; C[C@H]1COCCN1CC1(COc2nc(N3CC4CCC(C3)N4)c3ccc(-c4cc(O)cc5ccc(F)c(Cl)c45)c(F)c3n2)CC1 ; CCc1c(F)ccc2cc(O)cc(-c3ccc4c(N5CC6CCC(C5)N6)nc(OCC5(CN6CCOC[C@@H]6C)CC5)nc4c3F)c12]

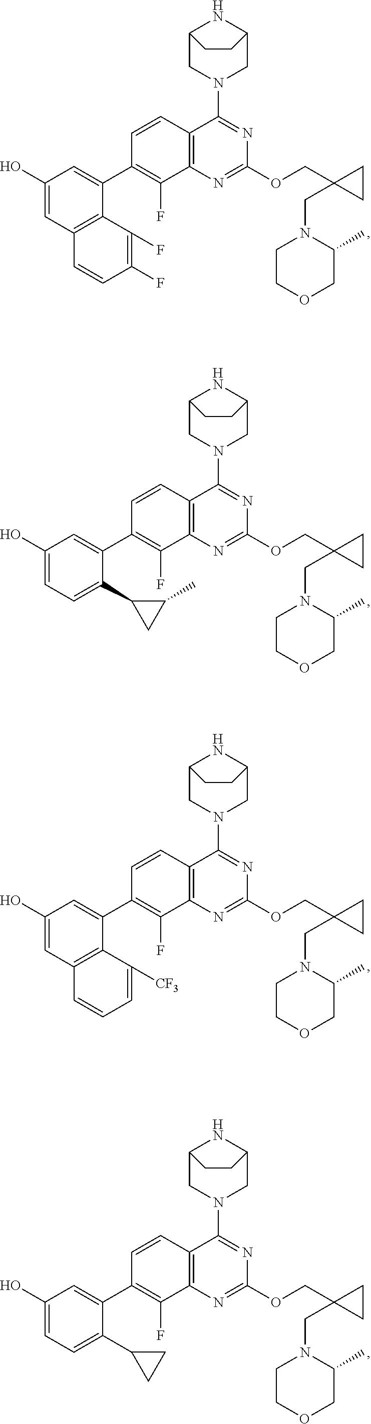 [SMILES: C[C@@H]1COCCN1CC1(COc2nc(N3CC4CCC(C3)N4)c3ccc(-c4cc(O)cc5ccc(F)c(F)c45)c(F)c3n2)CC1 ; C[C@@H]1C[C@H]1c1ccc(O)cc1-c1ccc2c(N3CC4CCC(C3)N4)nc(OCC3(CN4CCOC[C@H]4C)CC3)nc2c1F ; C[C@@H]1COCCN1CC1(COc2nc(N3CC4CCC(C3)N4)c3ccc(-c4cc(O)cc5cccc(C(F)(F)F)c45)c(F)c3n2)CC1 ; C[C@@H]1COCCN1CC1(COc2nc(N3CC4CCC(C3)N4)c3ccc(-c4cc(O)ccc4C4CC4)c(F)c3n2)CC1]

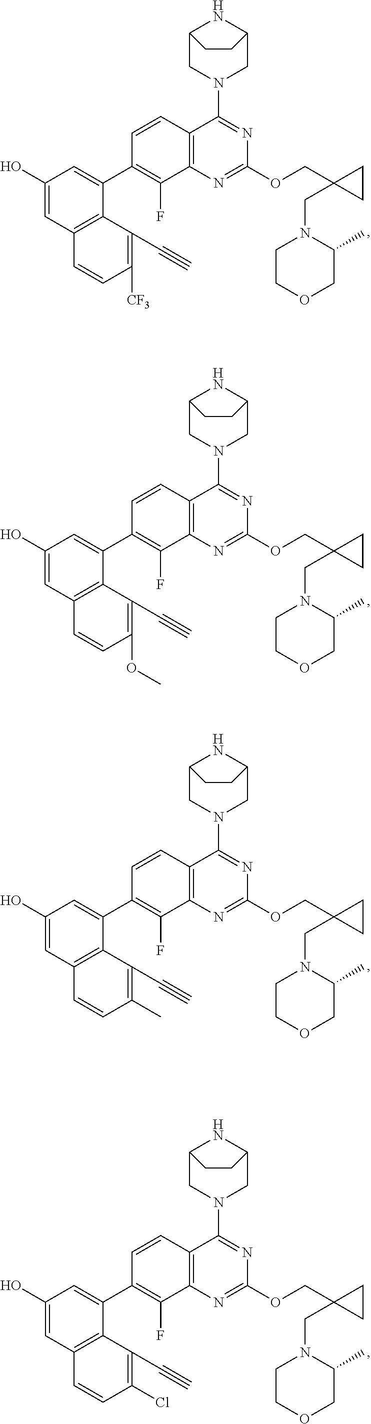 [SMILES: C#Cc1c(C(F)(F)F)ccc2cc(O)cc(-c3ccc4c(N5CC6CCC(C5)N6)nc(OCC5(CN6CCOC[C@H]6C)CC5)nc4c3F)c12 ; C#Cc1c(OC)ccc2cc(O)cc(-c3ccc4c(N5CC6CCC(C5)N6)nc(OCC5(CN6CCOC[C@H]6C)CC5)nc4c3F)c12 ; C#Cc1c(C)ccc2cc(O)cc(-c3ccc4c(N5CC6CCC(C5)N6)nc(OCC5(CN6CCOC[C@H]6C)CC5)nc4c3F)c12 ; C#Cc1c(Cl)ccc2cc(O)cc(-c3ccc4c(N5CC6CCC(C5)N6)nc(OCC5(CN6CCOC[C@H]6C)CC5)nc4c3F)c12]

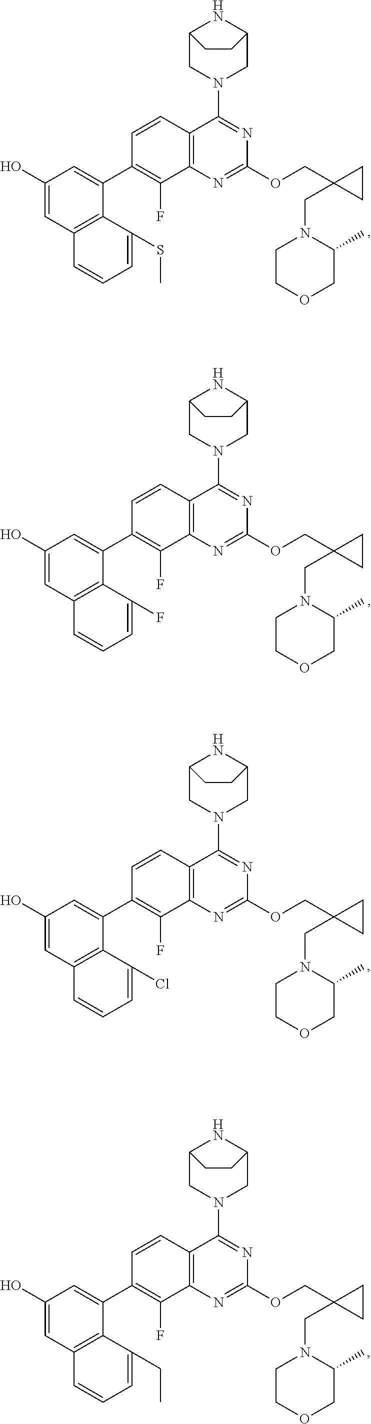 [SMILES: CSc1cccc2cc(O)cc(-c3ccc4c(N5CC6CCC(C5)N6)nc(OCC5(CN6CCOC[C@H]6C)CC5)nc4c3F)c12 ; C[C@@H]1COCCN1CC1(COc2nc(N3CC4CCC(C3)N4)c3ccc(-c4cc(O)cc5cccc(F)c45)c(F)c3n2)CC1 ; C[C@@H]1COCCN1CC1(COc2nc(N3CC4CCC(C3)N4)c3ccc(-c4cc(O)cc5cccc(Cl)c45)c(F)c3n2)CC1 ; CCc1cccc2cc(O)cc(-c3ccc4c(N5CC6CCC(C5)N6)nc(OCC5(CN6CCOC[C@H]6C)CC5)nc4c3F)c12]

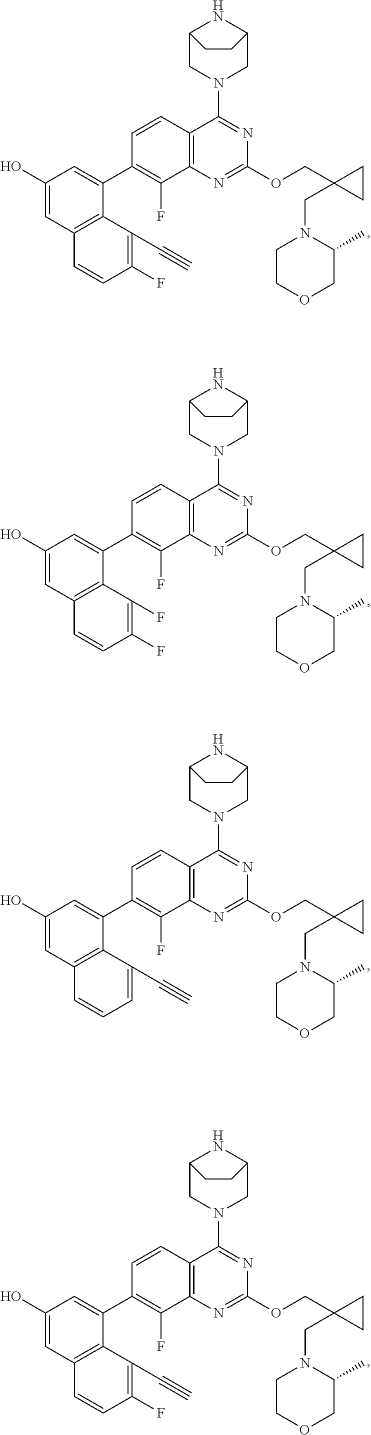 [SMILES: C#Cc1c(F)ccc2cc(O)cc(-c3ccc4c(N5CC6CCC(C5)N6)nc(OCC5(CN6CCOC[C@H]6C)CC5)nc4c3F)c12 ; C[C@@H]1COCCN1CC1(COc2nc(N3CC4CCC(C3)N4)c3ccc(-c4cc(O)cc5ccc(F)c(F)c45)c(F)c3n2)CC1 ; C#Cc1cccc2cc(O)cc(-c3ccc4c(N5CC6CCC(C5)N6)nc(OCC5(CN6CCOC[C@H]6C)CC5)nc4c3F)c12 ; C#Cc1c(F)ccc2cc(O)cc(-c3ccc4c(N5CC6CCC(C5)N6)nc(OCC5(CN6CCOC[C@H]6C)CC5)nc4c3F)c12]

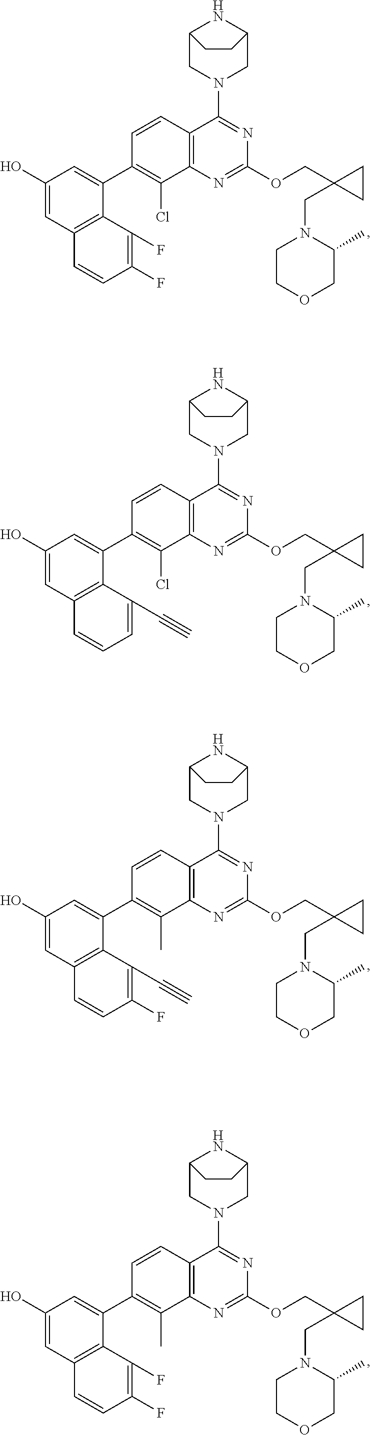 [SMILES: C[C@@H]1COCCN1CC1(COc2nc(N3CC4CCC(C3)N4)c3ccc(-c4cc(O)cc5ccc(F)c(F)c45)c(Cl)c3n2)CC1 ; C#Cc1cccc2cc(O)cc(-c3ccc4c(N5CC6CCC(C5)N6)nc(OCC5(CN6CCOC[C@H]6C)CC5)nc4c3Cl)c12 ; C#Cc1c(F)ccc2cc(O)cc(-c3ccc4c(N5CC6CCC(C5)N6)nc(OCC5(CN6CCOC[C@H]6C)CC5)nc4c3C)c12 ; Cc1c(-c2cc(O)cc3ccc(F)c(F)c23)ccc2c(N3CC4CCC(C3)N4)nc(OCC3(CN4CCOC[C@H]4C)CC3)nc12]

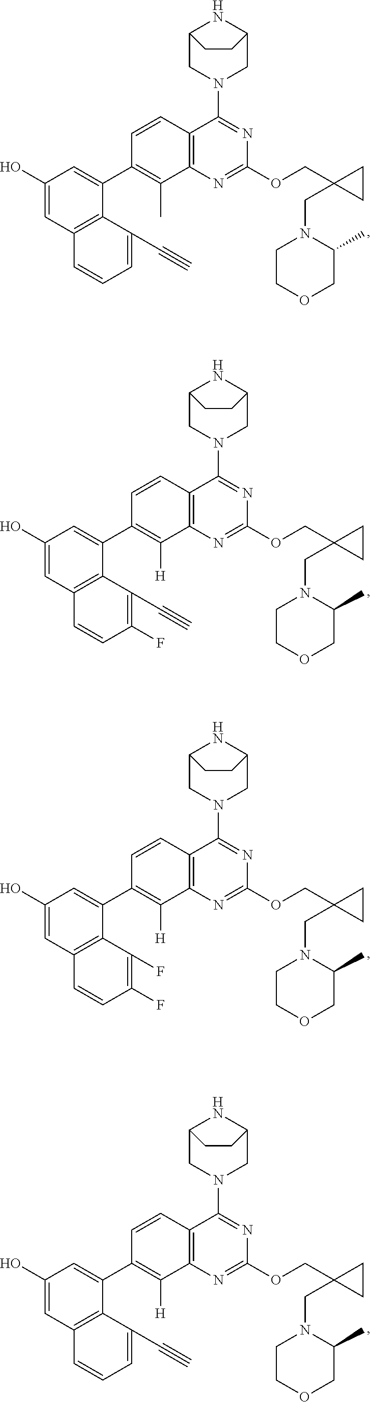 [SMILES: C#Cc1cccc2cc(O)cc(-c3ccc4c(N5CC6CCC(C5)N6)nc(OCC5(CN6CCOC[C@H]6C)CC5)nc4c3C)c12 ; C#Cc1c(F)ccc2cc(O)cc(-c3ccc4c(N5CC6CCC(C5)N6)nc(OCC5(CN6CCOC[C@@H]6C)CC5)nc4c3)c12 ; C[C@H]1COCCN1CC1(COc2nc(N3CC4CCC(C3)N4)c3ccc(-c4cc(O)cc5ccc(F)c(F)c45)cc3n2)CC1 ; C#Cc1cccc2cc(O)cc(-c3ccc4c(N5CC6CCC(C5)N6)nc(OCC5(CN6CCOC[C@@H]6C)CC5)nc4c3)c12]

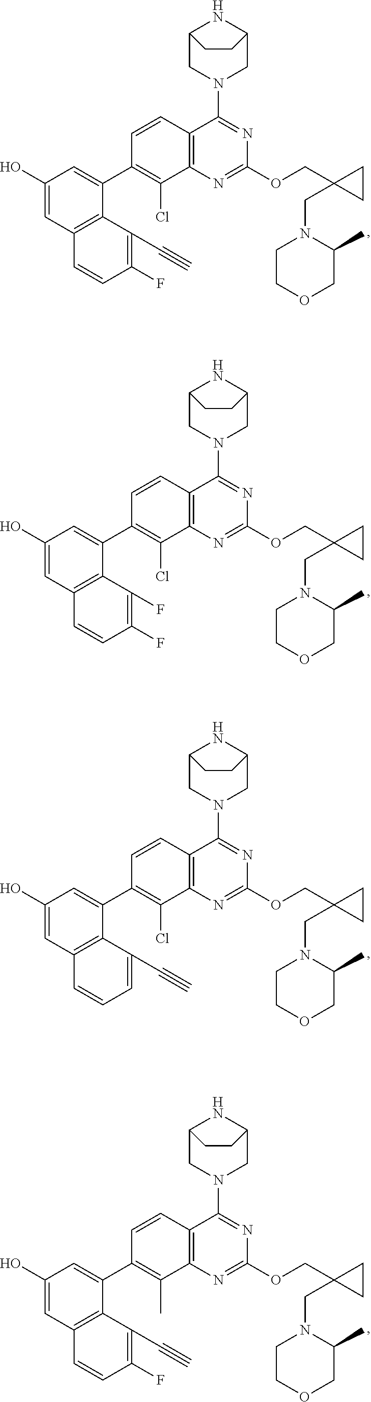 [SMILES: C#Cc1c(F)ccc2cc(O)cc(-c3ccc4c(N5CC6CCC(C5)N6)nc(OCC5(CN6CCOC[C@@H]6C)CC5)nc4c3Cl)c12 ; C[C@H]1COCCN1CC1(COc2nc(N3CC4CCC(C3)N4)c3ccc(-c4cc(O)cc5ccc(F)c(F)c45)c(Cl)c3n2)CC1 ; C#Cc1cccc2cc(O)cc(-c3ccc4c(N5CC6CCC(C5)N6)nc(OCC5(CN6CCOC[C@@H]6C)CC5)nc4c3Cl)c12 ; C#Cc1c(F)ccc2cc(O)cc(-c3ccc4c(N5CC6CCC(C5)N6)nc(OCC5(CN6CCOC[C@@H]6C)CC5)nc4c3C)c12]

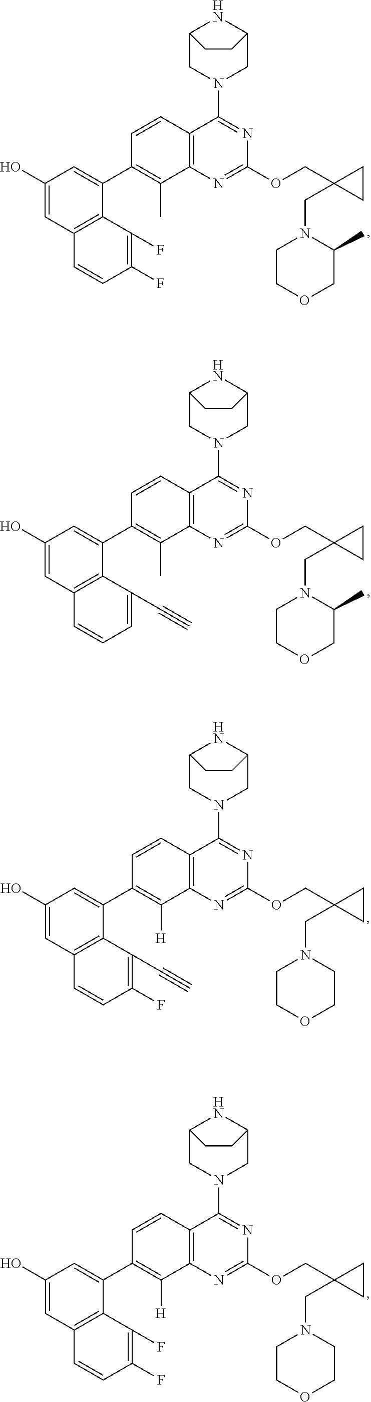 [SMILES: Cc1c(-c2cc(O)cc3ccc(F)c(F)c23)ccc2c(N3CC4CCC(C3)N4)nc(OCC3(CN4CCOC[C@@H]4C)CC3)nc12 ; C#Cc1cccc2cc(O)cc(-c3ccc4c(N5CC6CCC(C5)N6)nc(OCC5(CN6CCOC[C@@H]6C)CC5)nc4c3C)c12 ; C#Cc1c(F)ccc2cc(O)cc(-c3ccc4c(N5CC6CCC(C5)N6)nc(OCC5(CN6CCOCC6)CC5)nc4c3)c12 ; Oc1cc(-c2ccc3c(N4CC5CCC(C4)N5)nc(OCC4(CN5CCOCC5)CC4)nc3c2)c2c(F)c(F)ccc2c1]

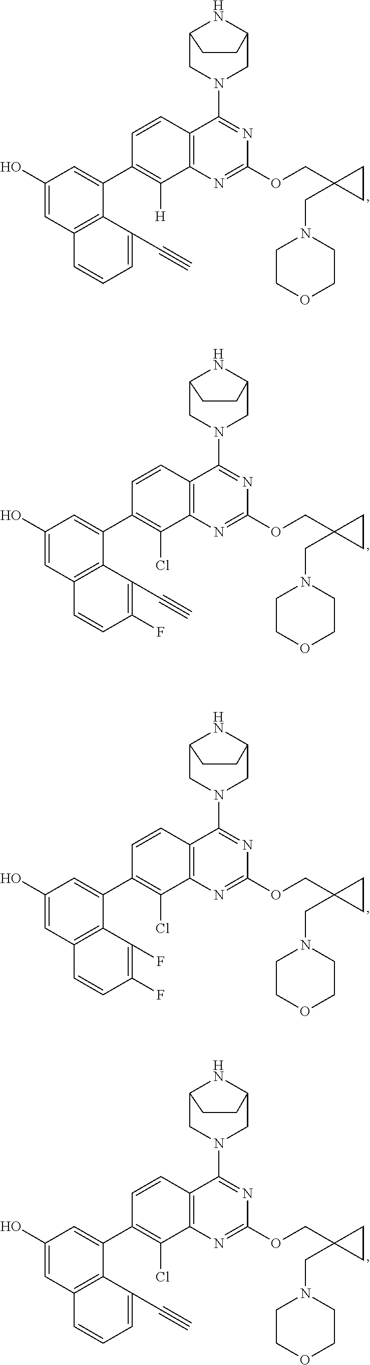 [SMILES: C#Cc1cccc2cc(O)cc(-c3ccc4c(N5CC6CCC(C5)N6)nc(OCC5(CN6CCOCC6)CC5)nc4c3)c12 ; C#Cc1c(F)ccc2cc(O)cc(-c3ccc4c(N5CC6CCC(C5)N6)nc(OCC5(CN6CCOCC6)CC5)nc4c3Cl)c12 ; Oc1cc(-c2ccc3c(N4CC5CCC(C4)N5)nc(OCC4(CN5CCOCC5)CC4)nc3c2Cl)c2c(F)c(F)ccc2c1 ; C#Cc1cccc2cc(O)cc(-c3ccc4c(N5CC6CCC(C5)N6)nc(OCC5(CN6CCOCC6)CC5)nc4c3Cl)c12]

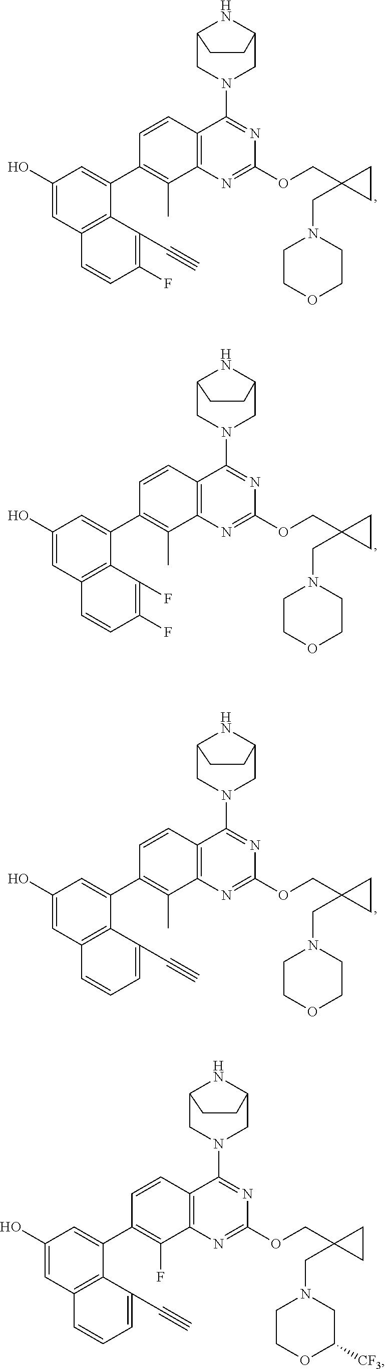 [SMILES: C#Cc1c(F)ccc2cc(O)cc(-c3ccc4c(N5CC6CCC(C5)N6)nc(OCC5(CN6CCOCC6)CC5)nc4c3C)c12 ; Cc1c(-c2cc(O)cc3ccc(F)c(F)c23)ccc2c(N3CC4CCC(C3)N4)nc(OCC3(CN4CCOCC4)CC3)nc12 ; C#Cc1cccc2cc(O)cc(-c3ccc4c(N5CC6CCC(C5)N6)nc(OCC5(CN6CCOCC6)CC5)nc4c3C)c12 ; C#Cc1cccc2cc(O)cc(-c3ccc4c(N5CC6CCC(C5)N6)nc(OCC5(CN6CCO[C@@H](C(F)(F)F)C6)CC5)nc4c3F)c12]

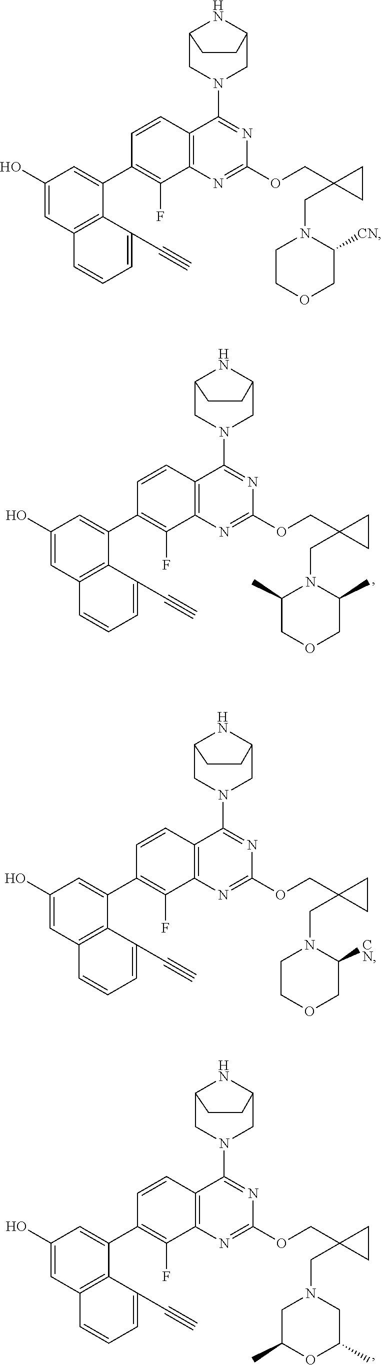 [SMILES: C#Cc1cccc2cc(O)cc(-c3ccc4c(N5CC6CCC(C5)N6)nc(OCC5(CN6CCOC[C@H]6C#N)CC5)nc4c3F)c12 ; C#Cc1cccc2cc(O)cc(-c3ccc4c(N5CC6CCC(C5)N6)nc(OCC5(CN6[C@H](C)COC[C@@H]6C)CC5)nc4c3F)c12 ; C#Cc1cccc2cc(O)cc(-c3ccc4c(N5CC6CCC(C5)N6)nc(OCC5(CN6CCOC[C@@H]6C#N)CC5)nc4c3F)c12 ; C#Cc1cccc2cc(O)cc(-c3ccc4c(N5CC6CCC(C5)N6)nc(OCC5(CN6C[C@H](C)O[C@@H](C)C6)CC5)nc4c3F)c12]

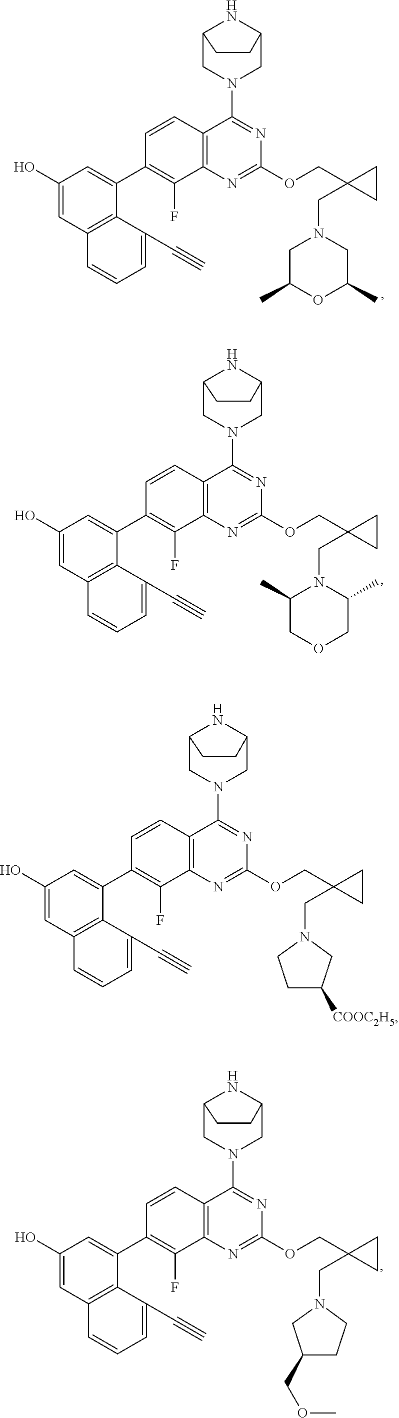

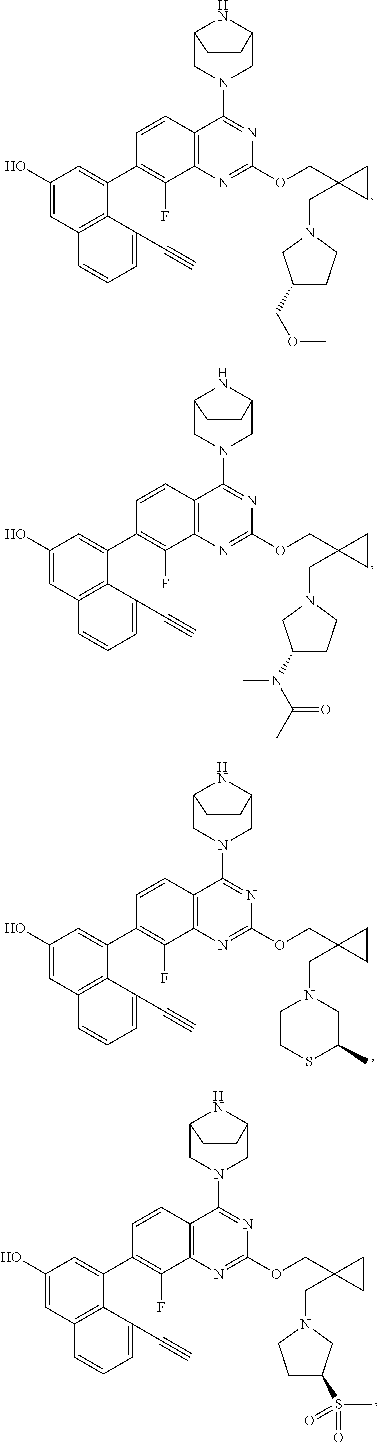 [SMILES: C#Cc1cccc2cc(O)cc(-c3ccc4c(N5CC6CCC(C5)N6)nc(OCC5(CN6CC[C@H](COC)C6)CC5)nc4c3F)c12 ; C#Cc1cccc2cc(O)cc(-c3ccc4c(N5CC6CCC(C5)N6)nc(OCC5(CN6CC[C@H](N(C)C(C)=O)C6)CC5)nc4c3F)c12 ; C#Cc1cccc2cc(O)cc(-c3ccc4c(N5CC6CCC(C5)N6)nc(OCC5(CN6CCS[C@H](C)C6)CC5)nc4c3F)c12 ; C#Cc1cccc2cc(O)cc(-c3ccc4c(N5CC6CCC(C5)N6)nc(OCC5(CN6CC[C@H](S(C)(=O)=O)C6)CC5)nc4c3F)c12]

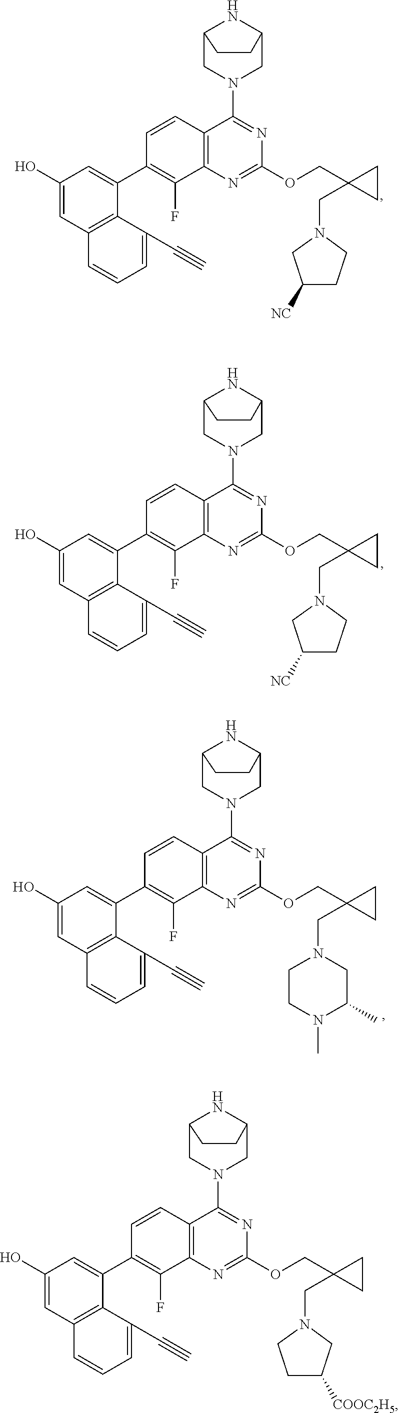

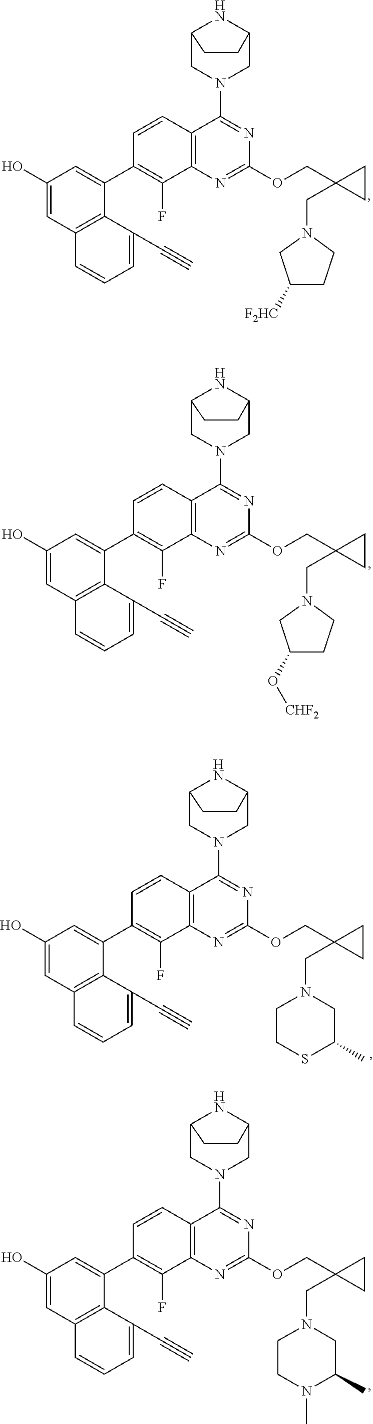 [SMILES: C#Cc1cccc2cc(O)cc(-c3ccc4c(N5CC6CCC(C5)N6)nc(OCC5(CN6CC[C@H](C(F)F)C6)CC5)nc4c3F)c12 ; C#Cc1cccc2cc(O)cc(-c3ccc4c(N5CC6CCC(C5)N6)nc(OCC5(CN6CC[C@H](OC(F)F)C6)CC5)nc4c3F)c12 ; C#Cc1cccc2cc(O)cc(-c3ccc4c(N5CC6CCC(C5)N6)nc(OCC5(CN6CCS[C@@H](C)C6)CC5)nc4c3F)c12 ; C#Cc1cccc2cc(O)cc(-c3ccc4c(N5CC6CCC(C5)N6)nc(OCC5(CN6CCN(C)[C@H](C)C6)CC5)nc4c3F)c12]

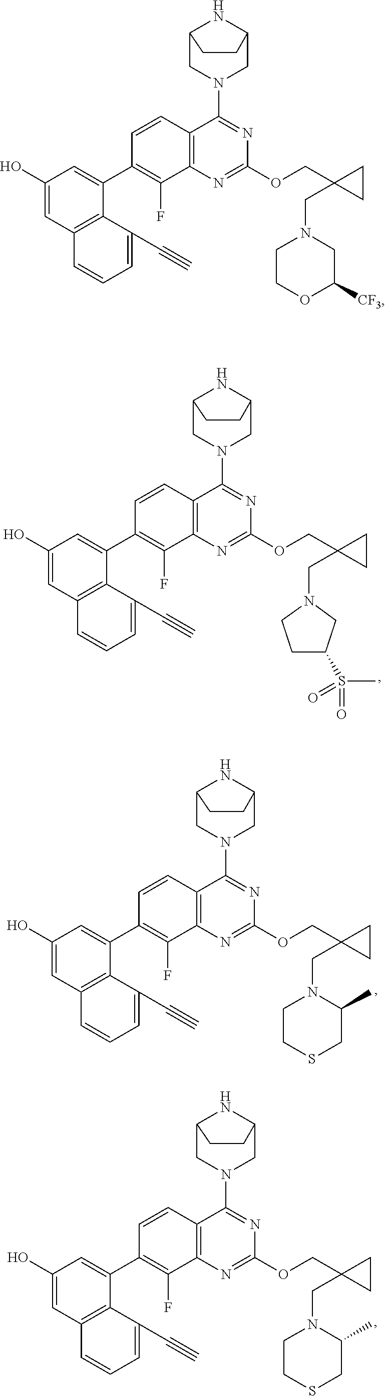 [SMILES: C#Cc1cccc2cc(O)cc(-c3ccc4c(N5CC6CCC(C5)N6)nc(OCC5(CN6CCO[C@H](C(F)(F)F)C6)CC5)nc4c3F)c12 ; C#Cc1cccc2cc(O)cc(-c3ccc4c(N5CC6CCC(C5)N6)nc(OCC5(CN6CC[C@@H](S(C)(=O)=O)C6)CC5)nc4c3F)c12 ; C#Cc1cccc2cc(O)cc(-c3ccc4c(N5CC6CCC(C5)N6)nc(OCC5(CN6CCSC[C@@H]6C)CC5)nc4c3F)c12 ; C#Cc1cccc2cc(O)cc(-c3ccc4c(N5CC6CCC(C5)N6)nc(OCC5(CN6CCSC[C@H]6C)CC5)nc4c3F)c12]

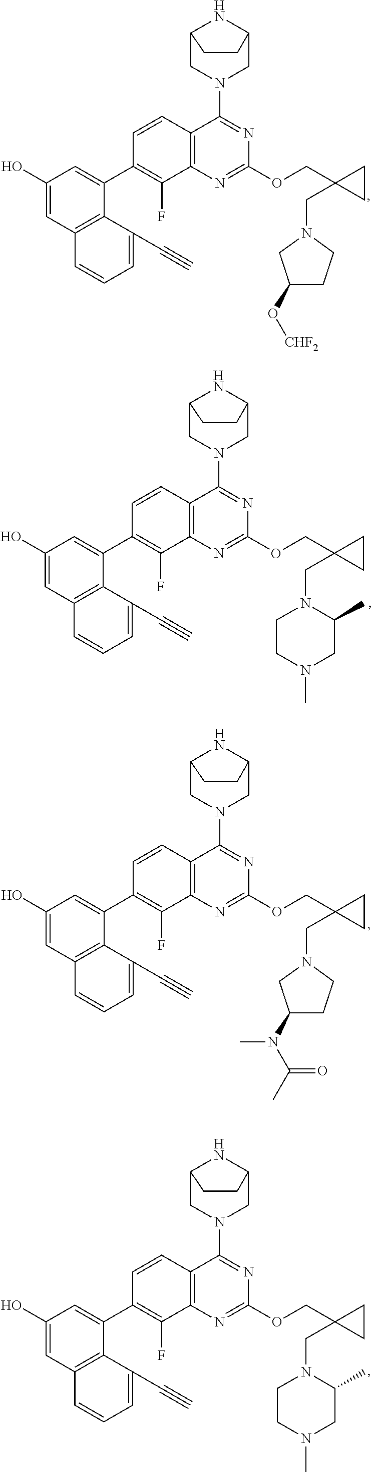 [SMILES: C#Cc1cccc2cc(O)cc(-c3ccc4c(N5CC6CCC(C5)N6)nc(OCC5(CN6CC[C@@H](OC(F)F)C6)CC5)nc4c3F)c12 ; C#Cc1cccc2cc(O)cc(-c3ccc4c(N5CC6CCC(C5)N6)nc(OCC5(CN6CCN(C)C[C@@H]6C)CC5)nc4c3F)c12 ; C#Cc1cccc2cc(O)cc(-c3ccc4c(N5CC6CCC(C5)N6)nc(OCC5(CN6CC[C@@H](N(C)C(C)=O)C6)CC5)nc4c3F)c12 ; C#Cc1cccc2cc(O)cc(-c3ccc4c(N5CC6CCC(C5)N6)nc(OCC5(CN6CCN(C)C[C@H]6C)CC5)nc4c3F)c12]

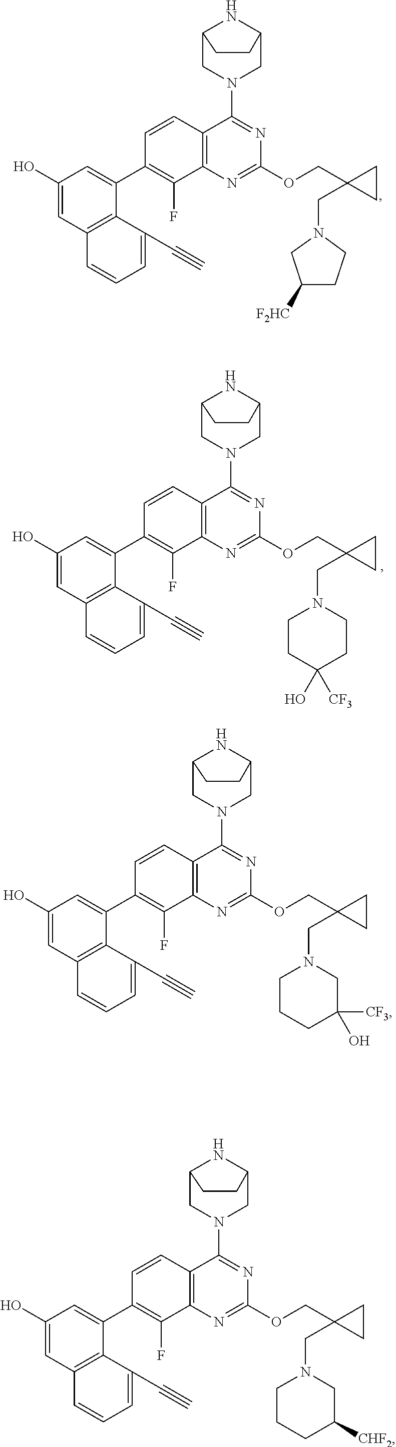 [SMILES: C#Cc1cccc2cc(O)cc(-c3ccc4c(N5CC6CCC(C5)N6)nc(OCC5(CN6CC[C@@H](C(F)F)C6)CC5)nc4c3F)c12 ; C#Cc1cccc2cc(O)cc(-c3ccc4c(N5CC6CCC(C5)N6)nc(OCC5(CN6CCC(O)(C(F)(F)F)CC6)CC5)nc4c3F)c12 ; C#Cc1cccc2cc(O)cc(-c3ccc4c(N5CC6CCC(C5)N6)nc(OCC5(CN6CCCC(O)(C(F)(F)F)C6)CC5)nc4c3F)c12 ; C#Cc1cccc2cc(O)cc(-c3ccc4c(N5CC6CCC(C5)N6)nc(OCC5(CN6CCC[C@H](C(F)F)C6)CC5)nc4c3F)c12]

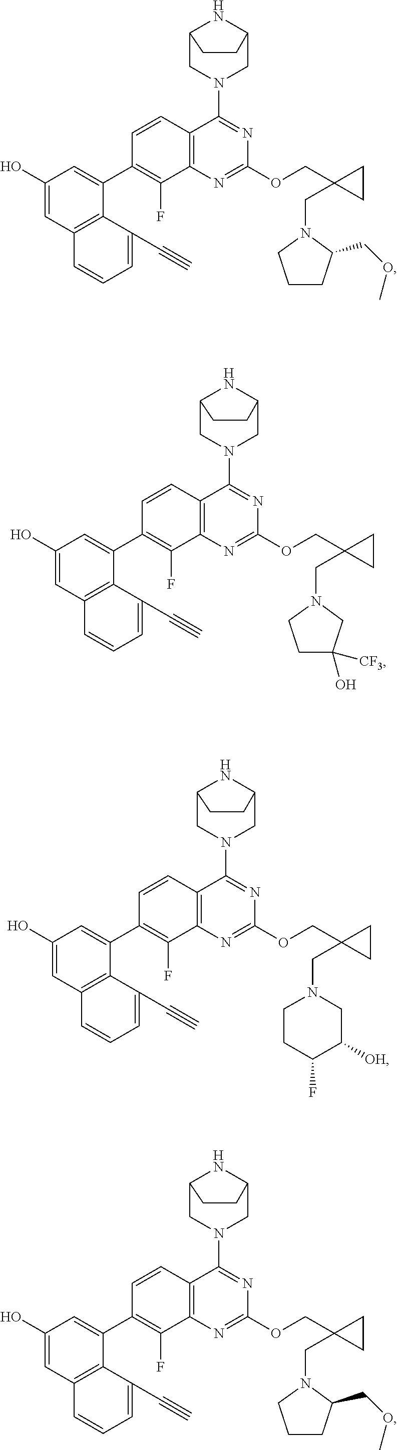 [SMILES: C#Cc1cccc2cc(O)cc(-c3ccc4c(N5CC6CCC(C5)N6)nc(OCC5(CN6CCC[C@H]6COC)CC5)nc4c3F)c12 ; C#Cc1cccc2cc(O)cc(-c3ccc4c(N5CC6CCC(C5)N6)nc(OCC5(CN6CCC(O)(C(F)(F)F)C6)CC5)nc4c3F)c12 ; C#Cc1cccc2cc(O)cc(-c3ccc4c(N5CC6CCC(C5)N6)nc(OCC5(CN6CC[C@@H](F)[C@@H](O)C6)CC5)nc4c3F)c12 ; C#Cc1cccc2cc(O)cc(-c3ccc4c(N5CC6CCC(C5)N6)nc(OCC5(CN6CCC[C@@H]6COC)CC5)nc4c3F)c12]

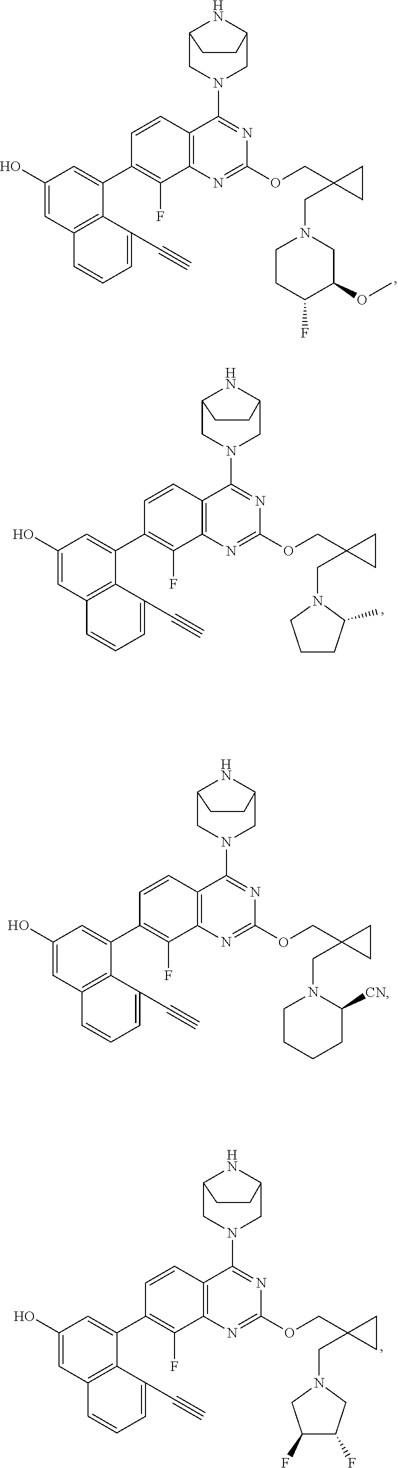 [SMILES: C#Cc1cccc2cc(O)cc(-c3ccc4c(N5CC6CCC(C5)N6)nc(OCC5(CN6CC[C@@H](F)[C@H](OC)C6)CC5)nc4c3F)c12 ; C#Cc1cccc2cc(O)cc(-c3ccc4c(N5CC6CCC(C5)N6)nc(OCC5(CN6CCC[C@H]6C)CC5)nc4c3F)c12 ; C#Cc1cccc2cc(O)cc(-c3ccc4c(N5CC6CCC(C5)N6)nc(OCC5(CN6CCCC[C@@H]6C#N)CC5)nc4c3F)c12 ; C#Cc1cccc2cc(O)cc(-c3ccc4c(N5CC6CCC(C5)N6)nc(OCC5(CN6C[C@H](F)[C@@H](F)C6)CC5)nc4c3F)c12]

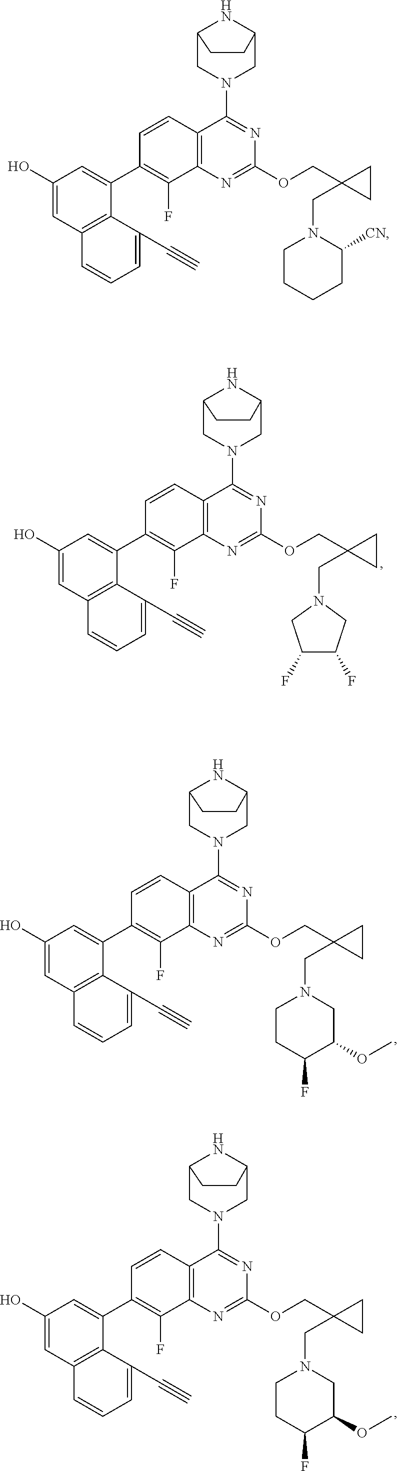 [SMILES: C#Cc1cccc2cc(O)cc(-c3ccc4c(N5CC6CCC(C5)N6)nc(OCC5(CN6CCCC[C@H]6C#N)CC5)nc4c3F)c12 ; C#Cc1cccc2cc(O)cc(-c3ccc4c(N5CC6CCC(C5)N6)nc(OCC5(CN6C[C@@H](F)[C@@H](F)C6)CC5)nc4c3F)c12 ; C#Cc1cccc2cc(O)cc(-c3ccc4c(N5CC6CCC(C5)N6)nc(OCC5(CN6CC[C@H](F)[C@@H](OC)C6)CC5)nc4c3F)c12 ; C#Cc1cccc2cc(O)cc(-c3ccc4c(N5CC6CCC(C5)N6)nc(OCC5(CN6CC[C@H](F)[C@H](OC)C6)CC5)nc4c3F)c12]

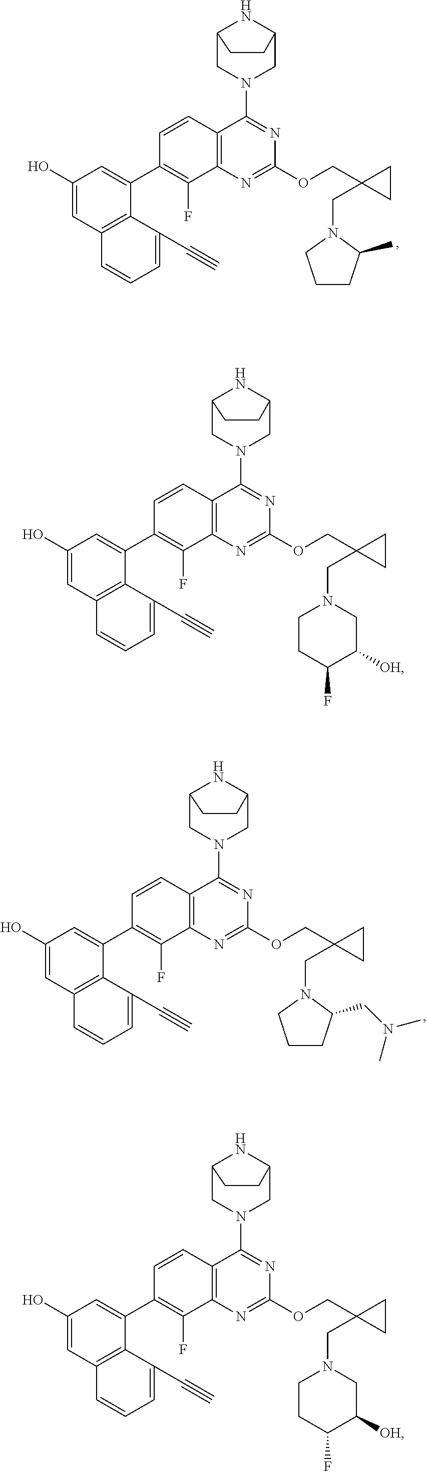 [SMILES: C#Cc1cccc2cc(O)cc(-c3ccc4c(N5CC6CCC(C5)N6)nc(OCC5(CN6CCC[C@@H]6C)CC5)nc4c3F)c12 ; C#Cc1cccc2cc(O)cc(-c3ccc4c(N5CC6CCC(C5)N6)nc(OCC5(CN6CC[C@H](F)[C@@H](O)C6)CC5)nc4c3F)c12 ; C#Cc1cccc2cc(O)cc(-c3ccc4c(N5CC6CCC(C5)N6)nc(OCC5(CN6CCC[C@H]6CN(C)C)CC5)nc4c3F)c12 ; C#Cc1cccc2cc(O)cc(-c3ccc4c(N5CC6CCC(C5)N6)nc(OCC5(CN6CC[C@@H](F)[C@H](O)C6)CC5)nc4c3F)c12]

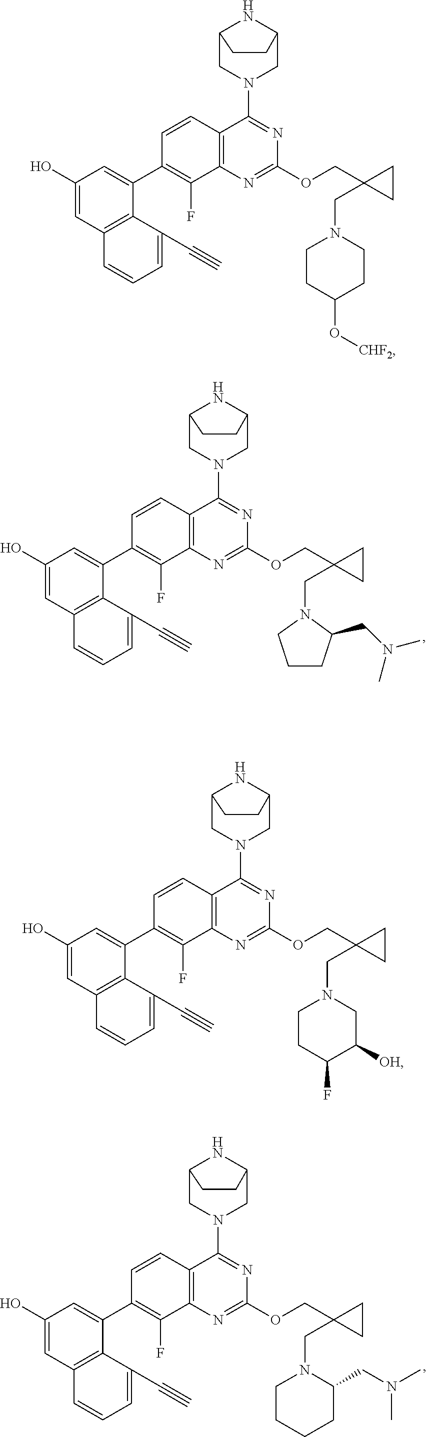 [SMILES: C#Cc1cccc2cc(O)cc(-c3ccc4c(N5CC6CCC(C5)N6)nc(OCC5(CN6CCC(OC(F)F)CC6)CC5)nc4c3F)c12 ; C#Cc1cccc2cc(O)cc(-c3ccc4c(N5CC6CCC(C5)N6)nc(OCC5(CN6CCC[C@@H]6CN(C)C)CC5)nc4c3F)c12 ; C#Cc1cccc2cc(O)cc(-c3ccc4c(N5CC6CCC(C5)N6)nc(OCC5(CN6CC[C@H](F)[C@H](O)C6)CC5)nc4c3F)c12 ; C#Cc1cccc2cc(O)cc(-c3ccc4c(N5CC6CCC(C5)N6)nc(OCC5(CN6CCCC[C@H]6CN(C)C)CC5)nc4c3F)c12]

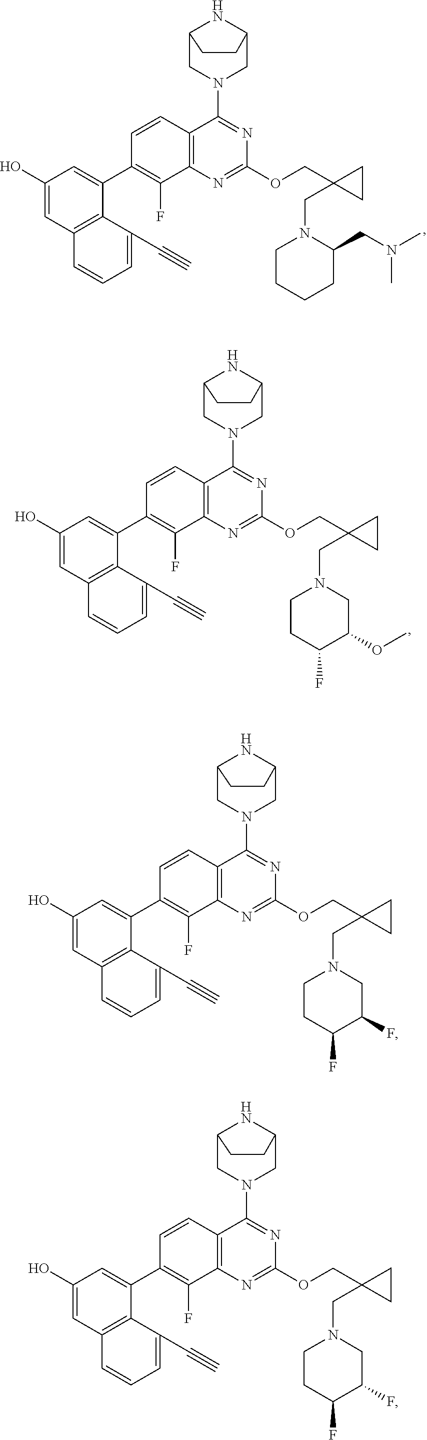 [SMILES: C#Cc1cccc2cc(O)cc(-c3ccc4c(N5CC6CCC(C5)N6)nc(OCC5(CN6CCCC[C@@H]6CN(C)C)CC5)nc4c3F)c12 ; C#Cc1cccc2cc(O)cc(-c3ccc4c(N5CC6CCC(C5)N6)nc(OCC5(CN6CC[C@@H](F)[C@@H](OC)C6)CC5)nc4c3F)c12 ; C#Cc1cccc2cc(O)cc(-c3ccc4c(N5CC6CCC(C5)N6)nc(OCC5(CN6CC[C@H](F)[C@H](F)C6)CC5)nc4c3F)c12 ; C#Cc1cccc2cc(O)cc(-c3ccc4c(N5CC6CCC(C5)N6)nc(OCC5(CN6CC[C@H](F)[C@@H](F)C6)CC5)nc4c3F)c12]

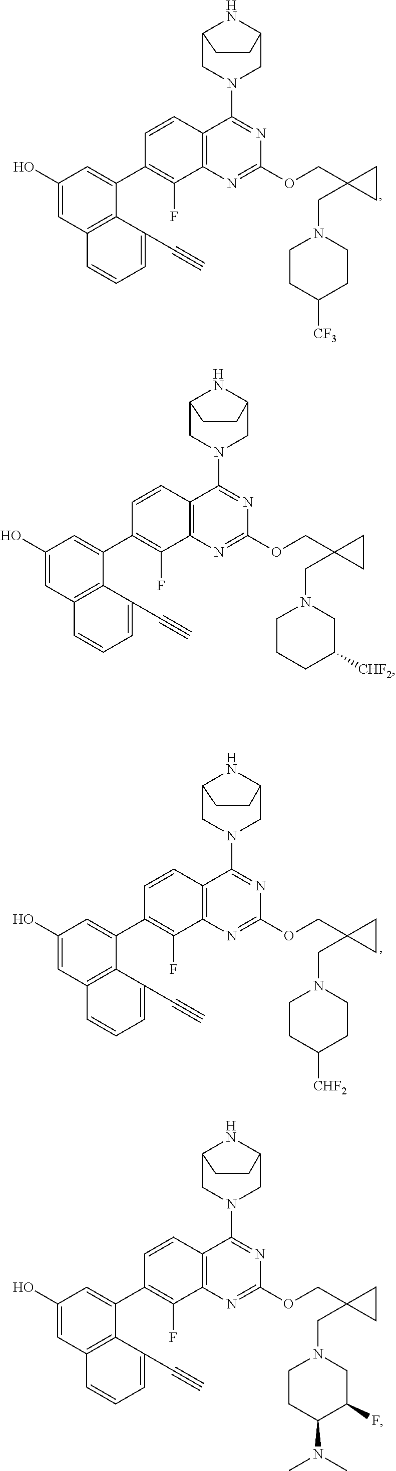 [SMILES: C#Cc1cccc2cc(O)cc(-c3ccc4c(N5CC6CCC(C5)N6)nc(OCC5(CN6CCC(C(F)(F)F)CC6)CC5)nc4c3F)c12 ; C#Cc1cccc2cc(O)cc(-c3ccc4c(N5CC6CCC(C5)N6)nc(OCC5(CN6CCC[C@@H](C(F)F)C6)CC5)nc4c3F)c12 ; C#Cc1cccc2cc(O)cc(-c3ccc4c(N5CC6CCC(C5)N6)nc(OCC5(CN6CCC(C(F)F)CC6)CC5)nc4c3F)c12 ; C#Cc1cccc2cc(O)cc(-c3ccc4c(N5CC6CCC(C5)N6)nc(OCC5(CN6CC[C@H](N(C)C)[C@H](F)C6)CC5)nc4c3F)c12]

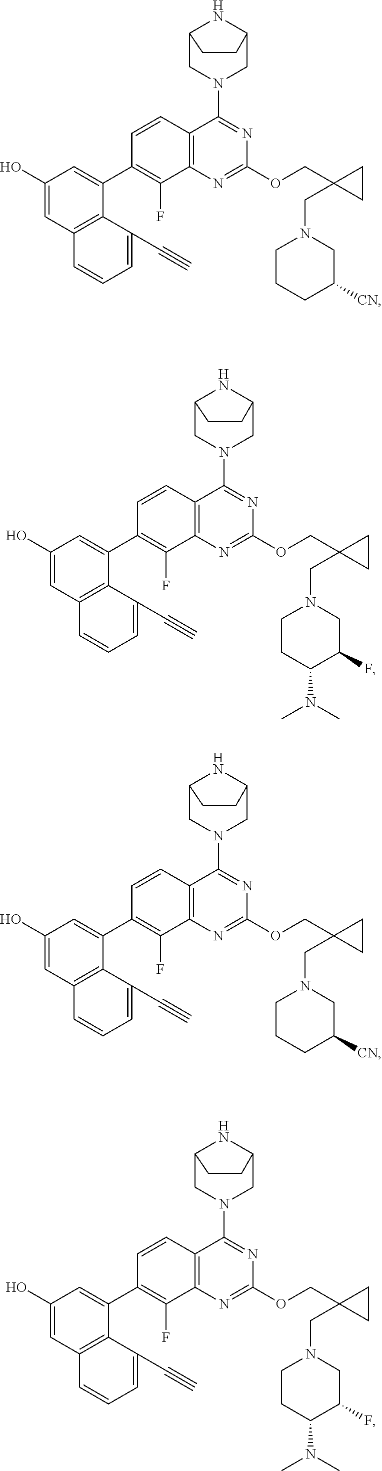 [SMILES: C#Cc1cccc2cc(O)cc(-c3ccc4c(N5CC6CCC(C5)N6)nc(OCC5(CN6CCC[C@@H](C#N)C6)CC5)nc4c3F)c12 ; C#Cc1cccc2cc(O)cc(-c3ccc4c(N5CC6CCC(C5)N6)nc(OCC5(CN6CC[C@@H](N(C)C)[C@H](F)C6)CC5)nc4c3F)c12 ; C#Cc1cccc2cc(O)cc(-c3ccc4c(N5CC6CCC(C5)N6)nc(OCC5(CN6CCC[C@H](C#N)C6)CC5)nc4c3F)c12 ; C#Cc1cccc2cc(O)cc(-c3ccc4c(N5CC6CCC(C5)N6)nc(OCC5(CN6CC[C@@H](N(C)C)[C@@H](F)C6)CC5)nc4c3F)c12]

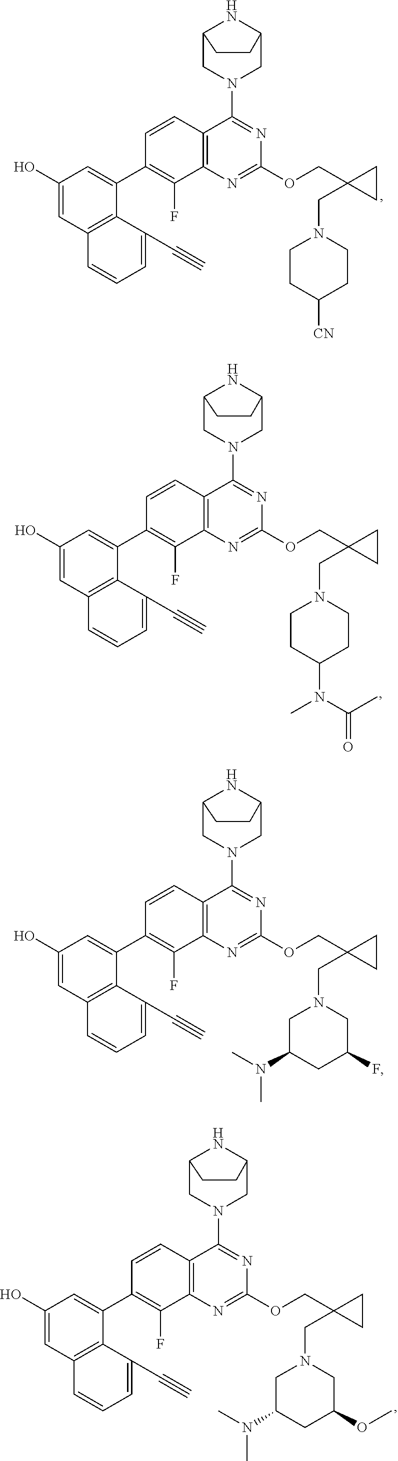 [SMILES: C#Cc1cccc2cc(O)cc(-c3ccc4c(N5CC6CCC(C5)N6)nc(OCC5(CN6CCC(C#N)CC6)CC5)nc4c3F)c12 ; C#Cc1cccc2cc(O)cc(-c3ccc4c(N5CC6CCC(C5)N6)nc(OCC5(CN6CCC(N(C)C(C)=O)CC6)CC5)nc4c3F)c12 ; C#Cc1cccc2cc(O)cc(-c3ccc4c(N5CC6CCC(C5)N6)nc(OCC5(CN6C[C@@H](F)C[C@@H](N(C)C)C6)CC5)nc4c3F)c12 ; C#Cc1cccc2cc(O)cc(-c3ccc4c(N5CC6CCC(C5)N6)nc(OCC5(CN6C[C@@H](OC)C[C@H](N(C)C)C6)CC5)nc4c3F)c12]

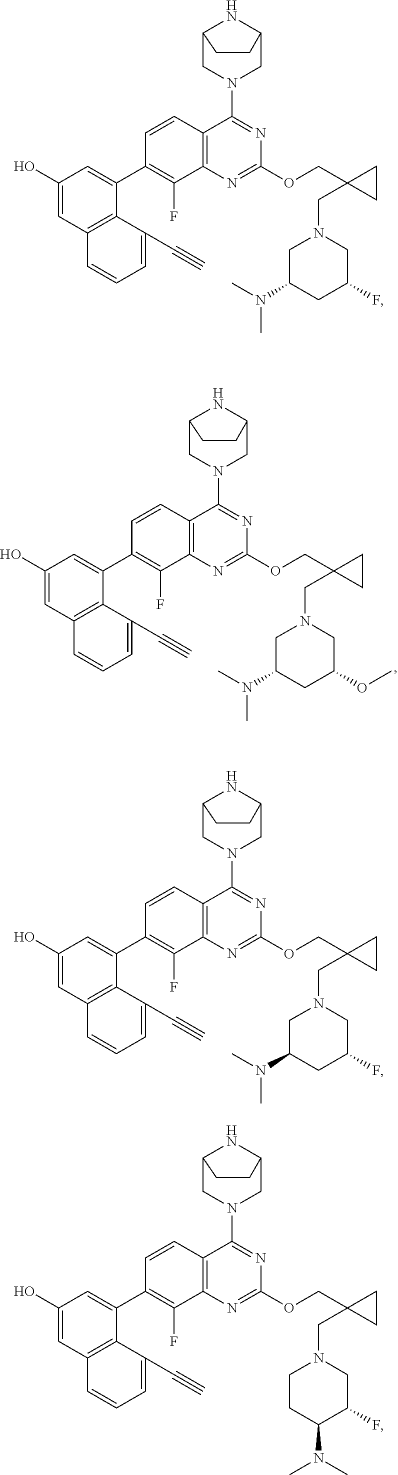 [SMILES: C#Cc1cccc2cc(O)cc(-c3ccc4c(N5CC6CCC(C5)N6)nc(OCC5(CN6C[C@H](F)C[C@H](N(C)C)C6)CC5)nc4c3F)c12 ; C#Cc1cccc2cc(O)cc(-c3ccc4c(N5CC6CCC(C5)N6)nc(OCC5(CN6C[C@H](OC)C[C@H](N(C)C)C6)CC5)nc4c3F)c12 ; C#Cc1cccc2cc(O)cc(-c3ccc4c(N5CC6CCC(C5)N6)nc(OCC5(CN6C[C@H](F)C[C@@H](N(C)C)C6)CC5)nc4c3F)c12 ; C#Cc1cccc2cc(O)cc(-c3ccc4c(N5CC6CCC(C5)N6)nc(OCC5(CN6CC[C@H](N(C)C)[C@@H](F)C6)CC5)nc4c3F)c12]

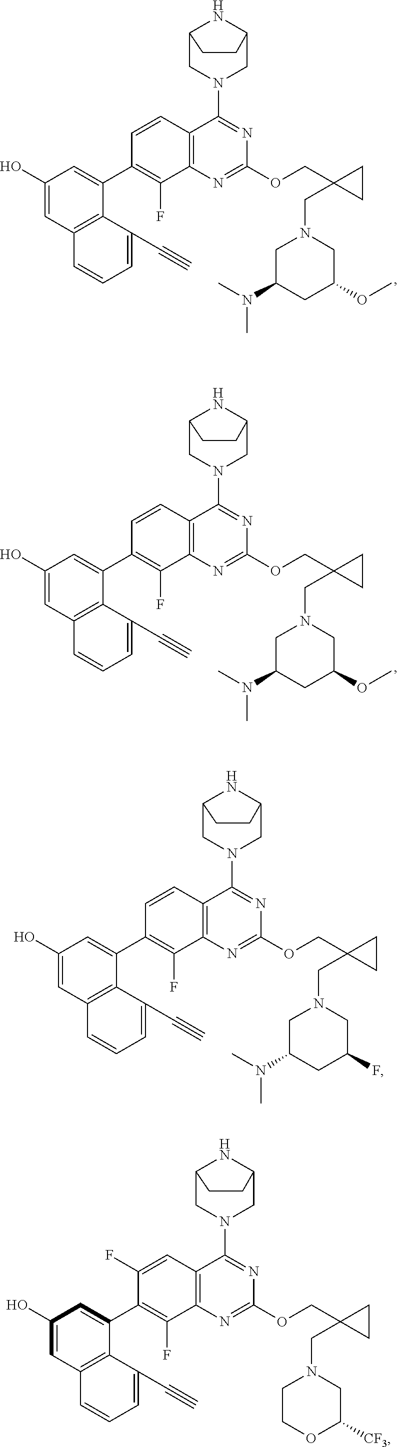

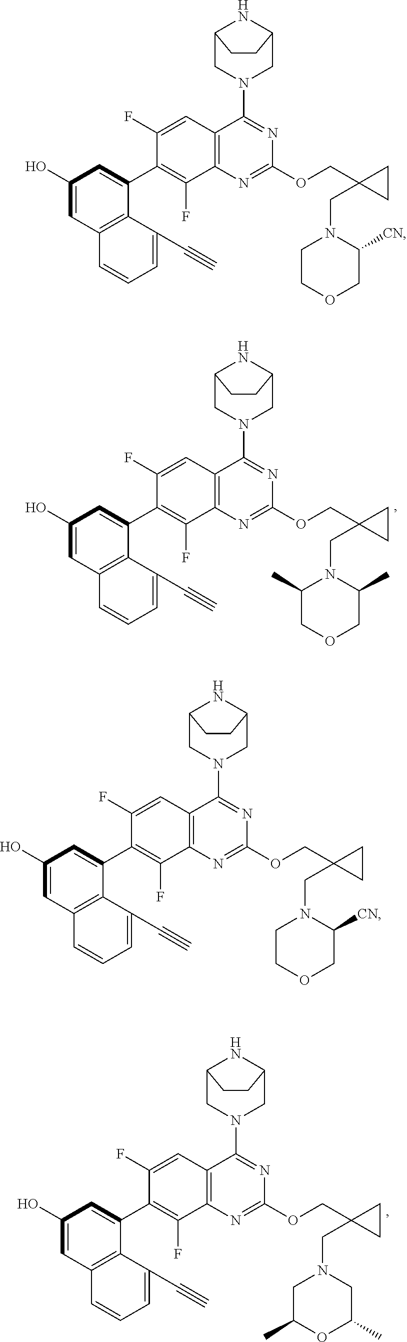

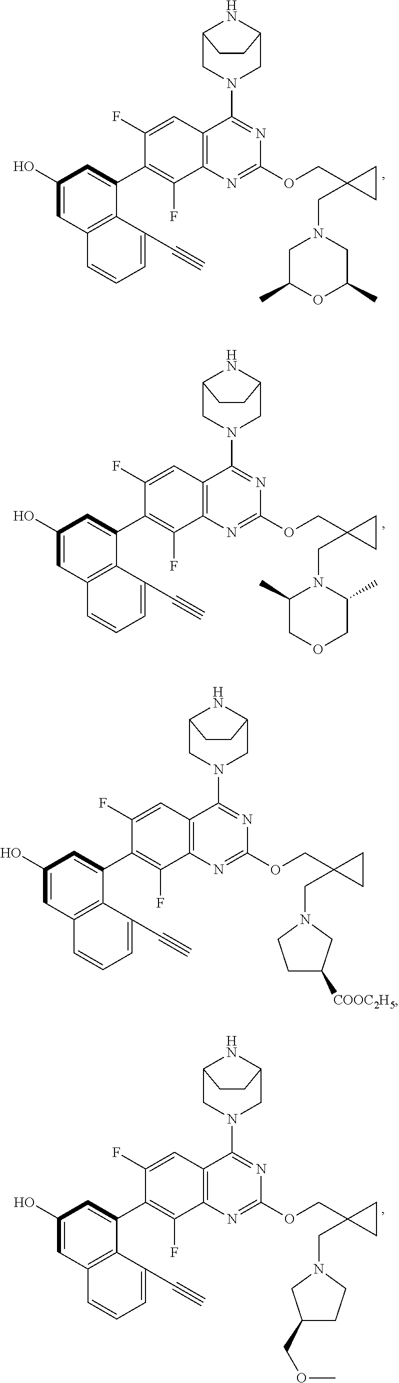

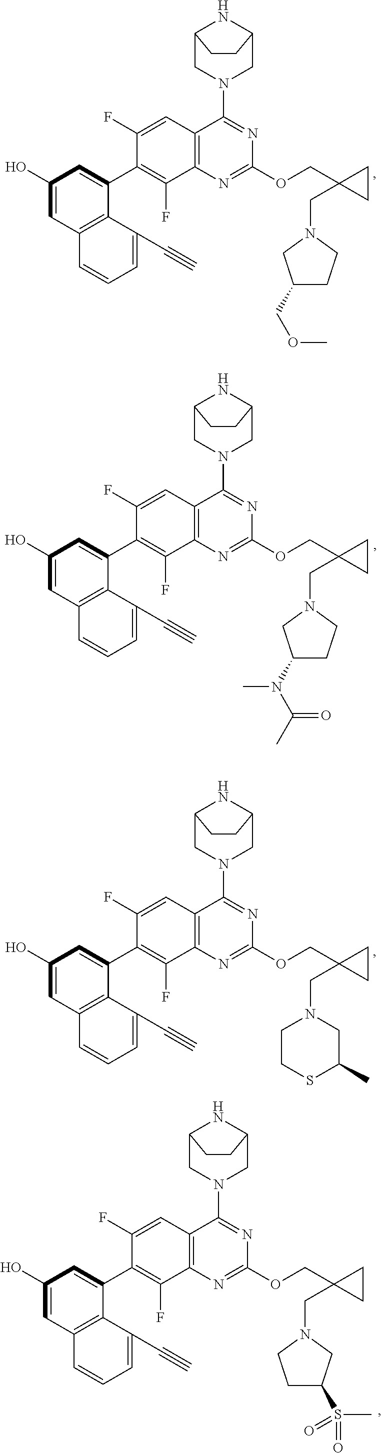

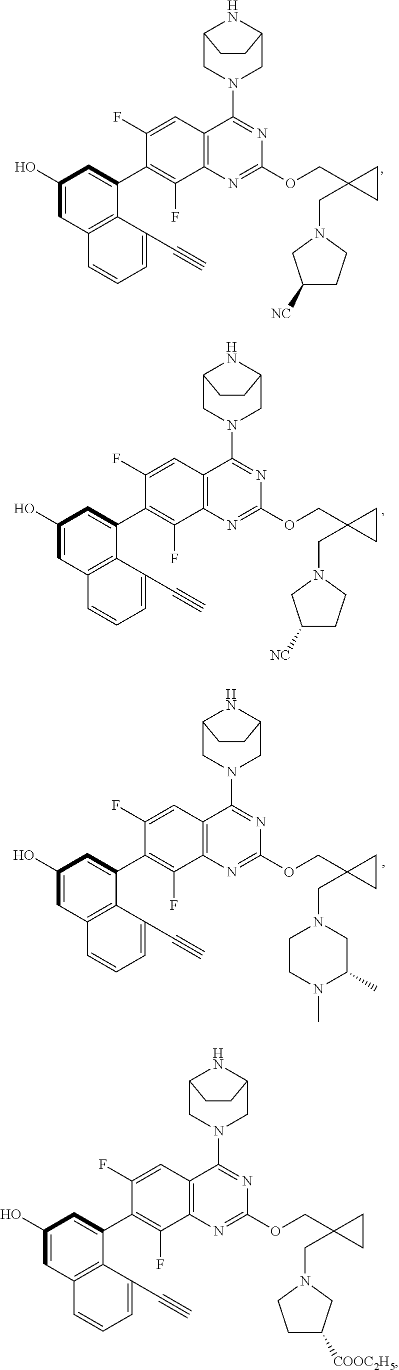

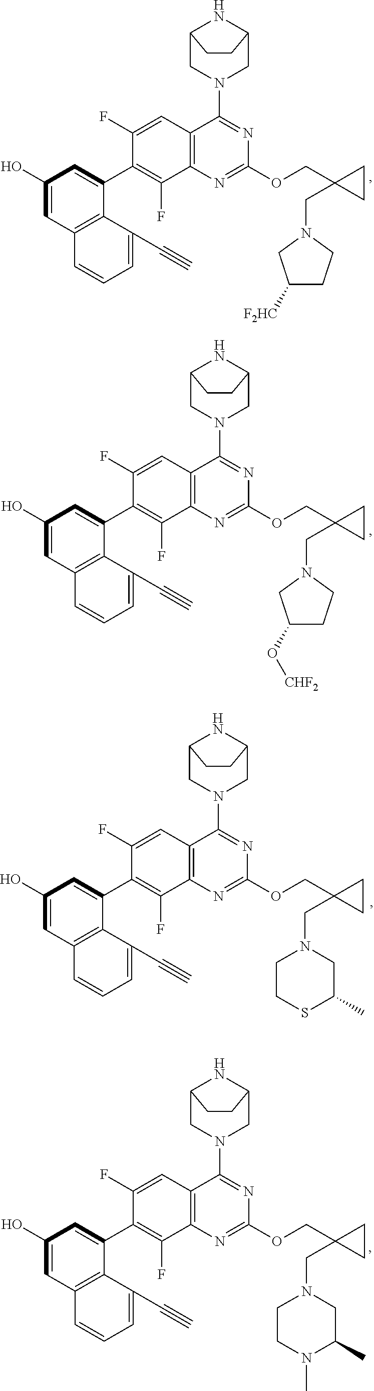

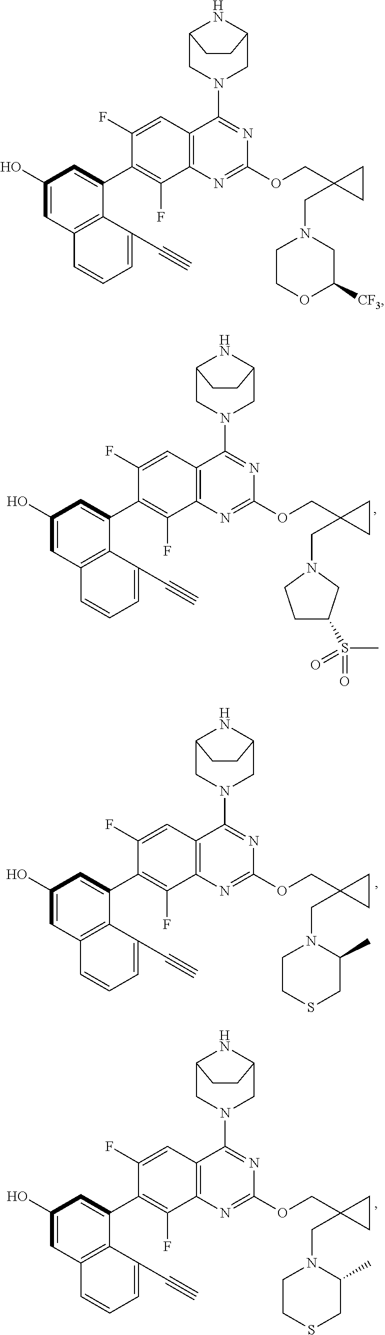

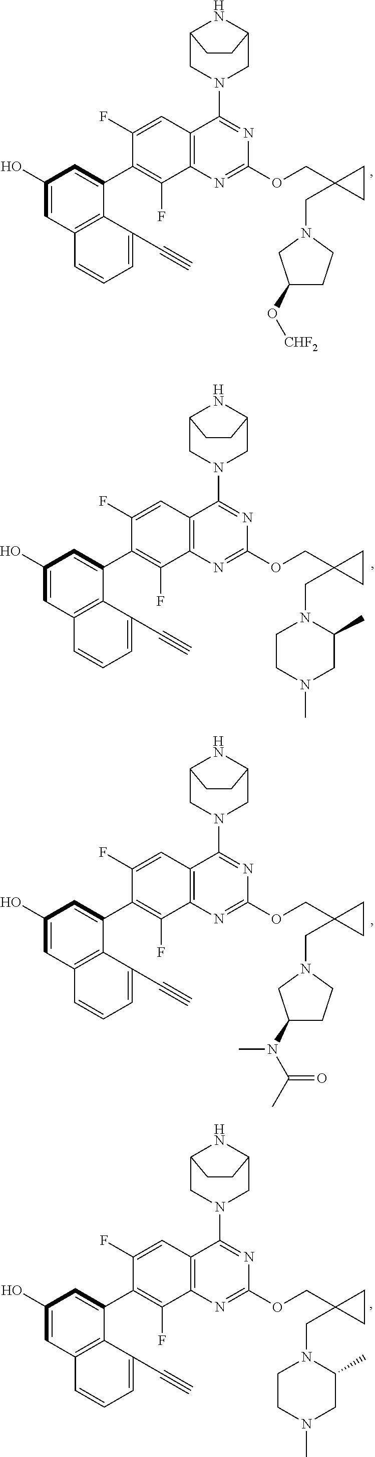

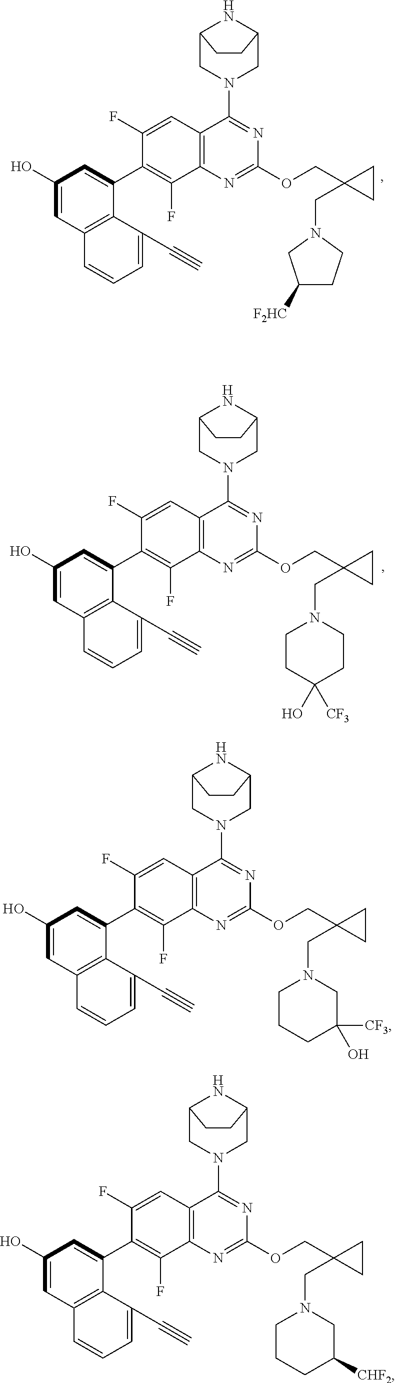

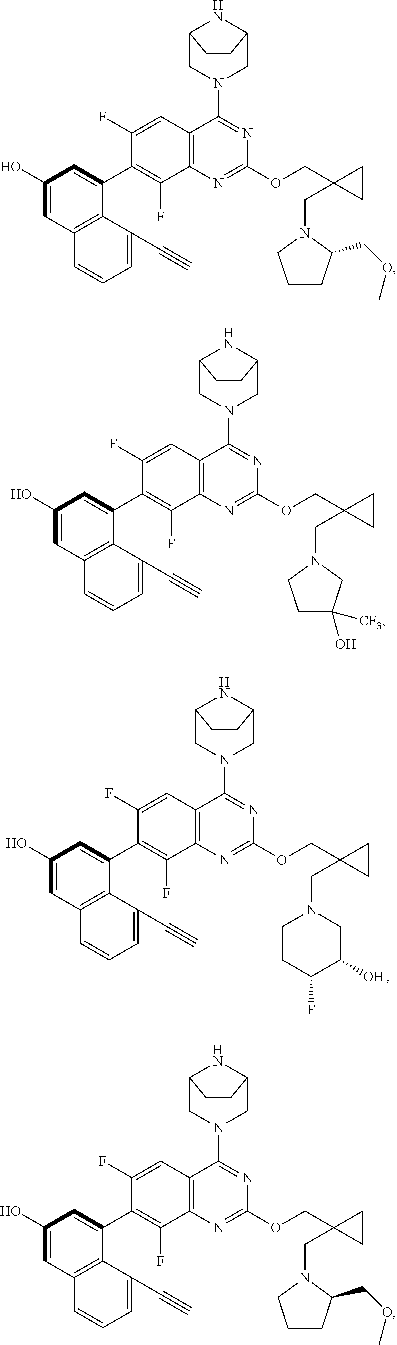

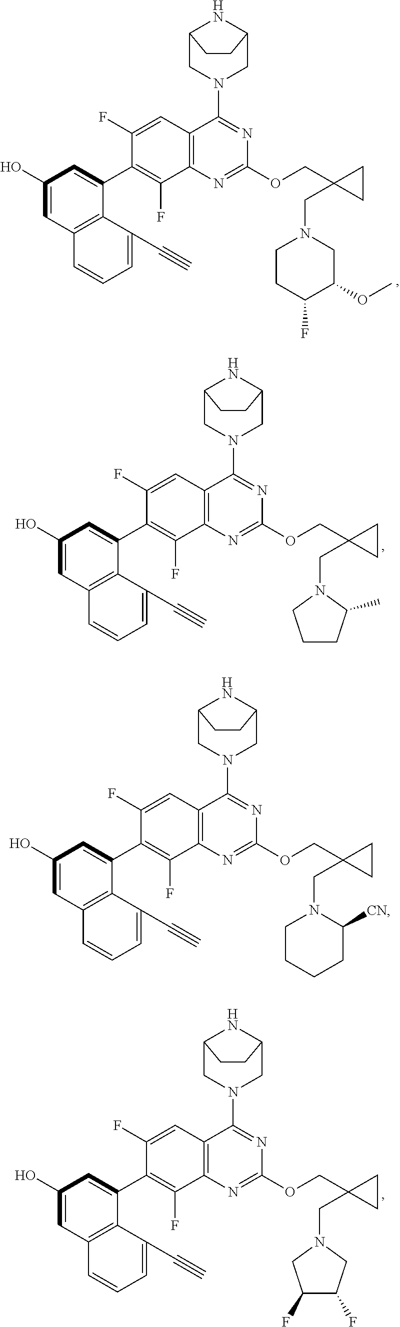

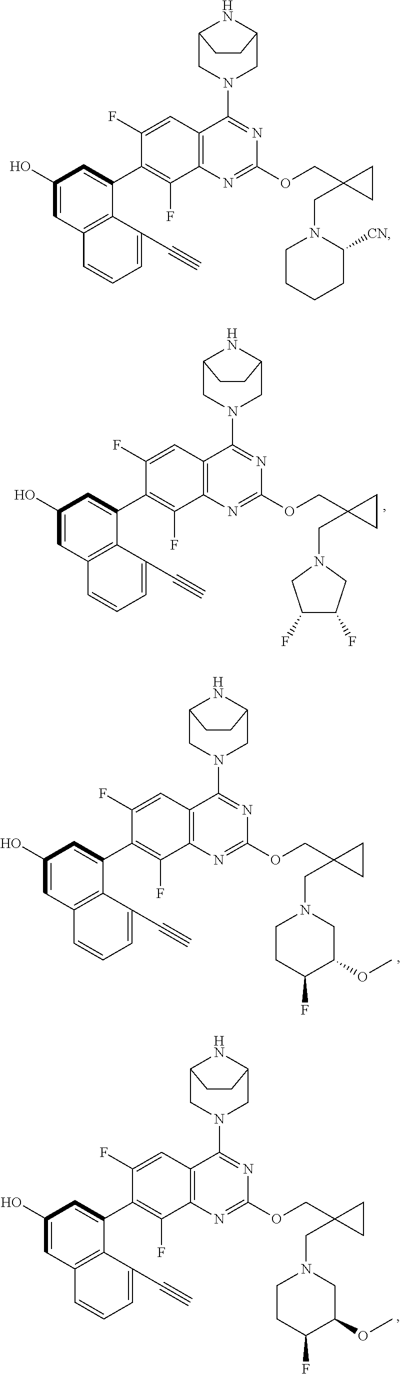

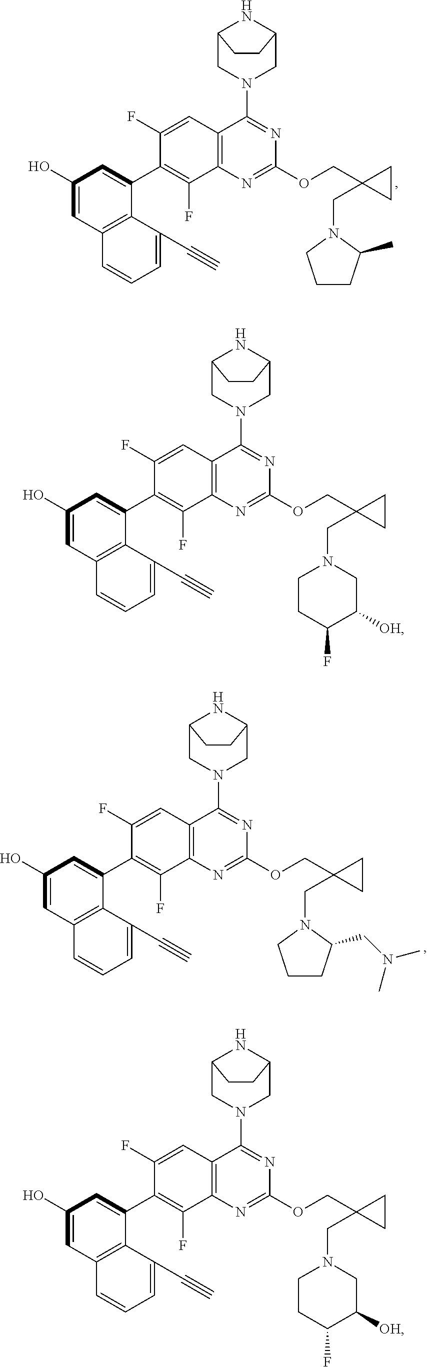

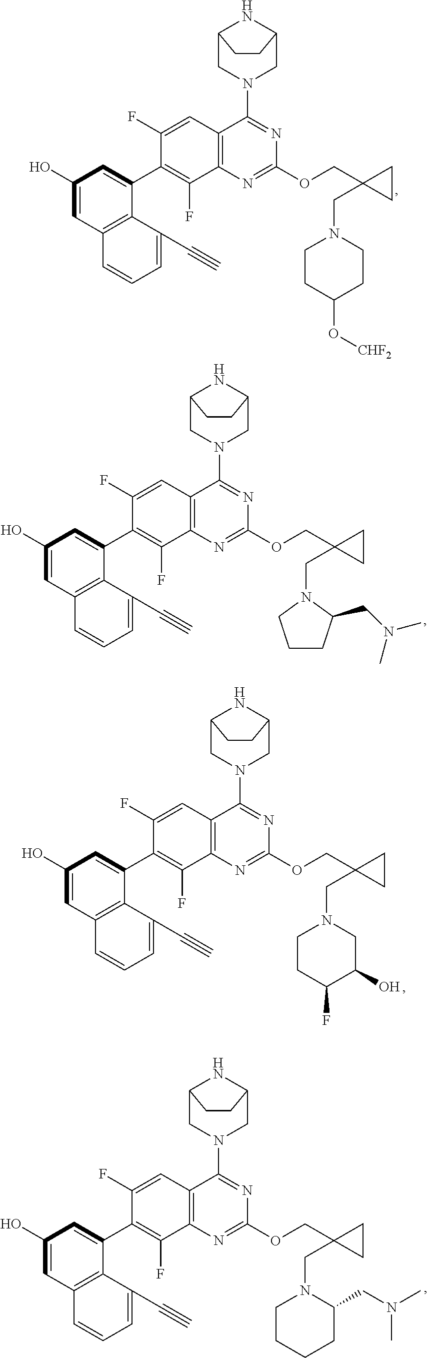

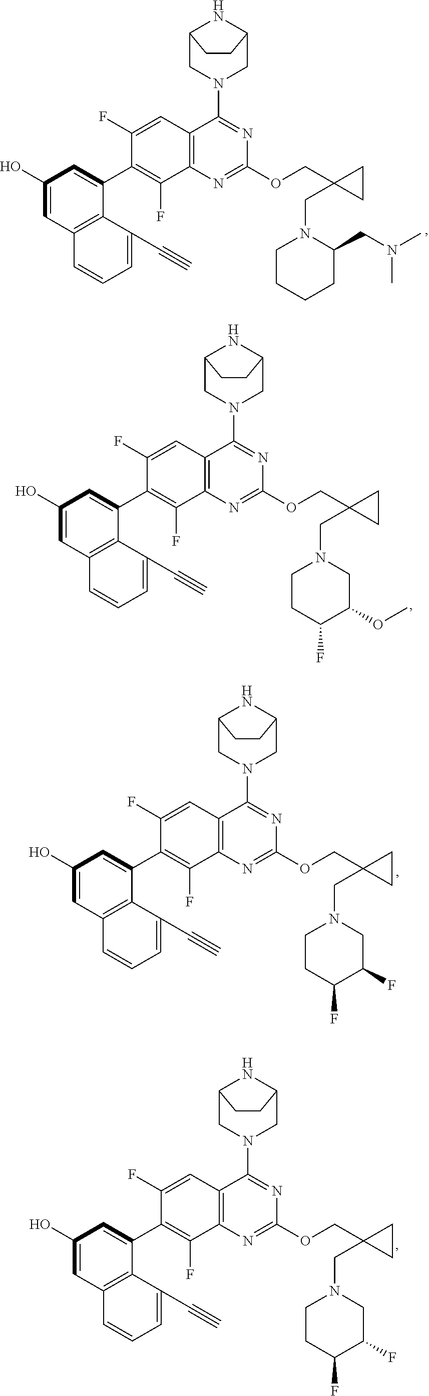

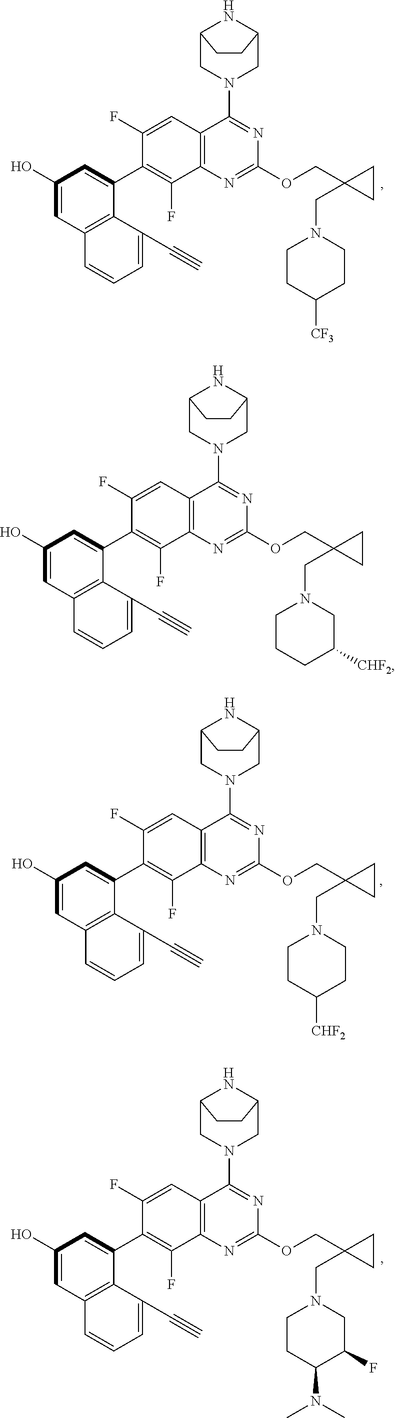

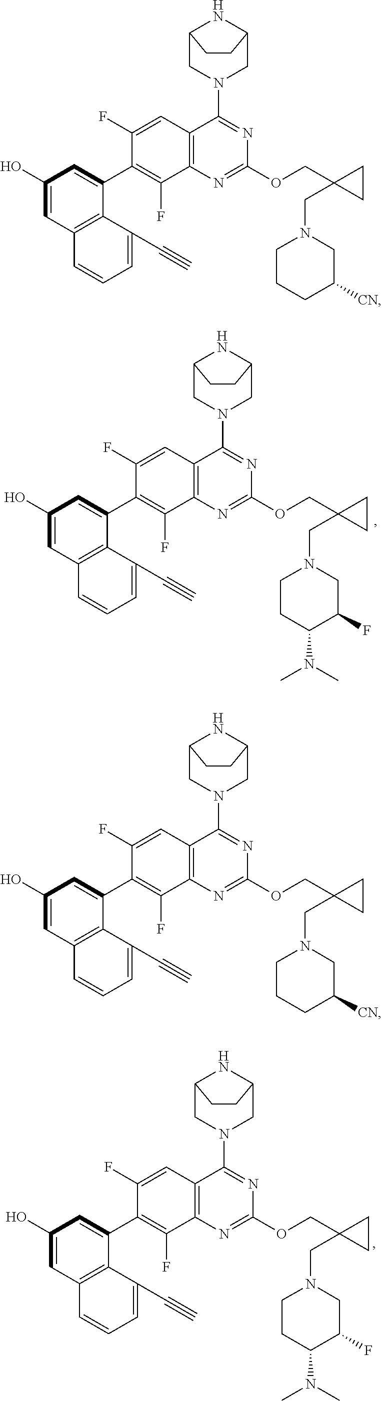

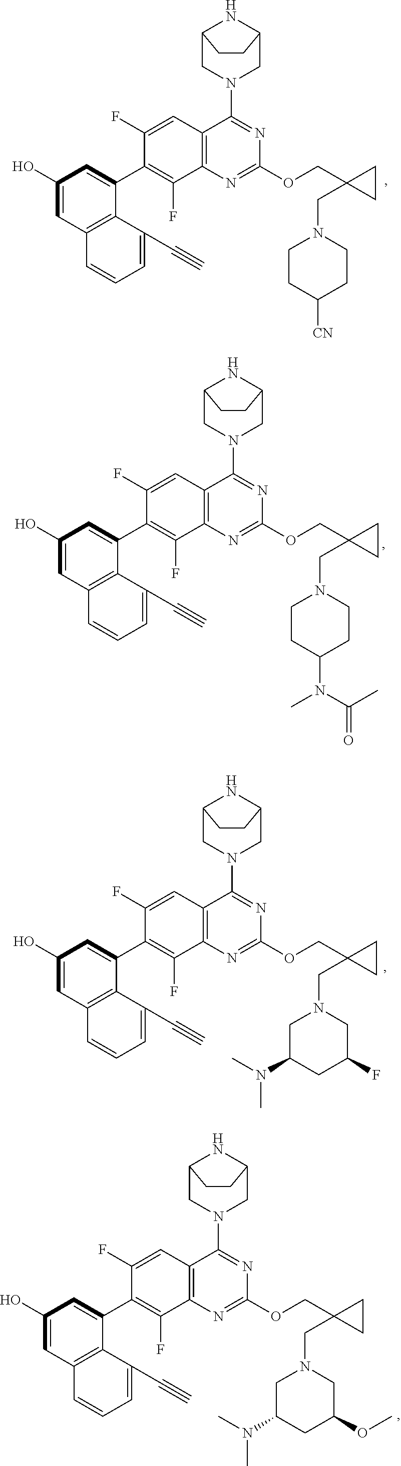

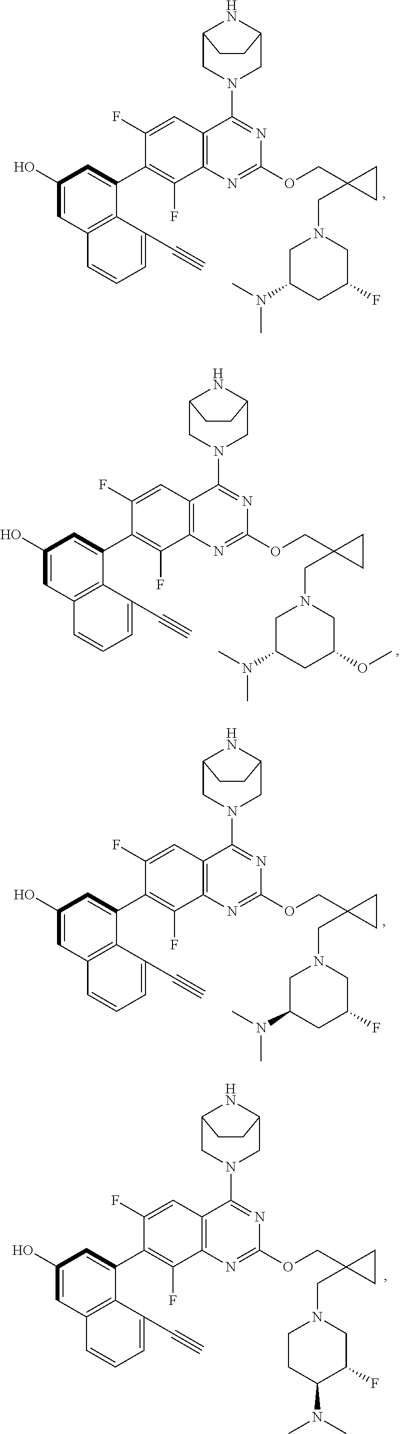

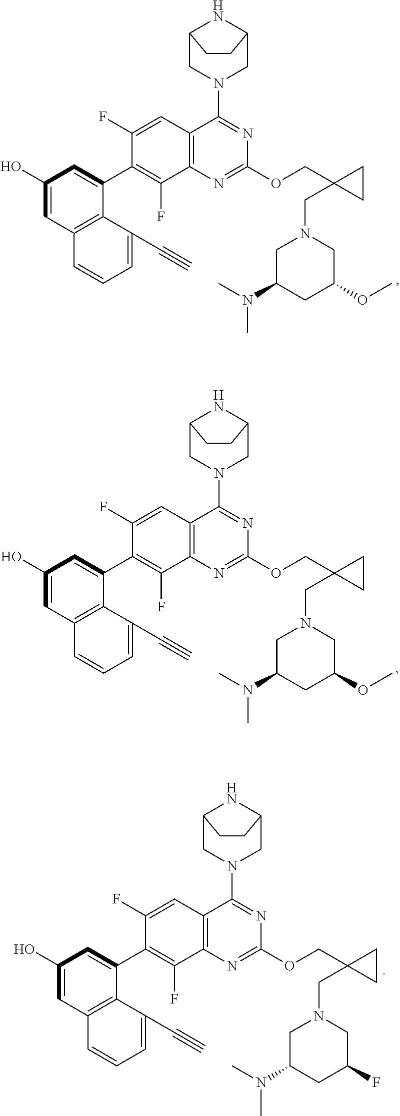

### Pharmacological Experiments

1. SOS1 Catalyzed Nucleotide Exchange Assay

GDP-loaded HIS-KRAS (G12D, aa 1-169) was pre-incubated with a compound in the presence of 10 nM GDP in a 384-well plate (Greiner) for 15 min, then purified SOS1 ExD (Flag tag, aa 564-1049), BODIPY™ FL GTP (Invitrogen) and MAb (monoclonal antibody) Anti 6HIS-Tb cryptate Gold (Cisbio) were added to the assay wells (Final concentration: 1.5 nM GDP-loaded HIS-KRAS (G12D), 5 nM GDP, 0.5 μM SOS1 ExD, 80 nM BODIPY™ FL GTP, 52.5 ng/mL MAb Anti 6HIS-Tb cryptate Gold) and incubated for 4 hours at 25° C. Wells containing same percent of DMSO served as vehicle control, and wells without KRAS served as low control. TR-FRET signals were read on Tecan Spark multimode microplate reader. The parameters were F486: Excitation 340 nm, Emission 486 nm, Lag time 100 μs, Integration time 200 μs; F515: Excitation 340 nm, Emission 515 nm, Lag time 100 μs, Integration time 200 μs. TR-FRET ratios for each individual wells were calculated by equation: TR-FRET ratio=(Signal F515/Signal F486)*10000. The percent of activation of compounds treated wells were normalized between vehicle control and low control (% Activation=(TR-FRET ratio_{Compound treated}−TR-FRET ratio_{Low control})/(TR-FRET ratio_{Vehicle control}−TR-FRET ratio_{Low control})*100%). Then the data were analyzed either by fitting a 4-parameter logistic model or by Excel to calculate IC₅₀ values.

2. GTP-KRAS and cRAF Interaction Assay

GppNp-loaded HIS-KRAS (G12D, aa 1-169) was pre-incubated with a compound in the presence of 200 M GTP in a 384-well plate (Greiner) for 15 min, then cRAF RBD (GST tag, aa 50-132, CreativeBioMart), MAb Anti GST-d2 (Cisbio) and MAb Anti 6HIS-Tb cryptate Gold (Cisbio) were added to the assay wells (Final concentration: 2.0 nM GppNp-loaded HIS-KRAS (G12D), 100 μM GTP, 35 nM cRAF RBD, 1 μg/mL MAb Anti GST-d2, 52.5 ng/mL MAb Anti 6HIS-Tb cryptate Gold) and incubated for 2 hours at 25° C. Wells containing same percent of DMSO served as vehicle control, and wells without KRAS served as low control. HTRF signals were read on Tecan Spark multimode microplate reader and HTRF ratios were calculated under manufacturer's instructions. The percent of activation of compounds treated wells were normalized between vehicle control and low control (% Activation=(HTRF ratio_{Compound treated}−HTRF ratio_{Low control})/(HTRF ratio_{Vehicle control}−HTRF ratio_{Low control})*100). Then the data were analyzed either by fitting a 4-parameter logistic model or by Excel to calculate IC₅₀ values.

3. Phospho-ERK1/2(THR202/TYR204) HTRF Assay

AGS cells expressing KRAS G12D were cultured in F12K medium (Gibco) containing 10% fetal bovine serum (Gibco). The AGS cells in culture medium were seeded in 96-well plates at a concentration of 40,000 cells/well and then put in a 37° C./5% CO₂ cell incubator to incubate overnight. The next day, culture medium was removed and the compound diluted in assay medium (F12K, 0.1% FBS) was added in each well. After 2 hours incubation in a 37° C./5% CO₂ cell incubator, the assay medium in 96-well plates was removed, then 50 μL of 1× blocking reagent-supplemented lysis buffer (Cisbio) was added and the plates were incubated at 25° C. for 45 min with shaking. 10 μL of cell lysates from the 96-well plates were transferred to a 384-well plate (Greiner) containing 2.5 μL/well HTRF® pre-mixed antibodies (Cisbio 64AERPEH). Incubate 4 hours at 25° C. and then read HTRF signals on Tecan Spark multimode microplate reader. The data were analyzed using a 4-parameter logistic model to calculate IC₅₀ values.

4. Cell Growth Inhibition Assay

AGS cultured in F12K medium (Gibco) containing 10% fetal bovine serum (Gibco). Cells in culture medium were plated in 96-well plates at a concentration of 500 cells/well (100 L/well) and allowed to attach overnight.

AsPC-1 cultured in RPMI-1640 medium (Gibco) containing 10% fetal bovine serum (Gibco). Cells in culture medium were plated in 96-well plates at a concentration of 1000 cells/well (100 L/well) and allowed to attach overnight.

The next day, compounds were diluted in culture medium and added to the plates. After 6 days incubation in a 37° C./5% CO₂ cell incubator, the cell viability was detected by CellTiter-Glo® Luminescent Cell Viability Assay kit (Promega). Luminescent signals were read on Tecan Spark multimode microplate reader and analyzed using a 4-parameter logistic model to calculate IC₅₀ values.

The results of the above experiments were shown in the table:

| | | GTP-KRAS/ | | AGS Cell | AsPC-1 Cell |
|---|---|---|---|---|---|
| | Nucleotide | cRAF | AGS | growth | growth |
| Compound | exchange | interaction | p-ERK | inhibition | inhibition |
| No. | IC₅₀ (μM) | | | | |
| 33 | 0.014 | 0.269 | | 0.103 | 0.0803 |
| 34 | 0.00873 | 0.0126 | 0.00216 | 0.0111 | 0.0191 |
| 35 | 0.0104 | 0.113 | 0.0322 | 0.00858 | 0.0993 |
| 35A | 0.651 | | | | |
| 35B | 0.00594 | 0.111 | 0.0062 | 0.0134 | 0.0395 |
| 36 | 0.0328 | | | | |
| 37 | 0.0514 | | | | |
| 38 | 0.0138 | 0.126 | 0.0198 | 0.051 | 0.141 |
| 38A | ~1 | 8.304 | | | |
| 38B | 0.000727 | 0.0132 | 0.0124 | 0.0105 | 0.158 |
| 39 | 0.322 | 1.412 | | | |
| 40 | 0.0277 | 0.0743 | 0.00372 | 0.0154 | 0.0555 |
| 40A | 0.0921 | 1.612 | | | |
| 40B | 0.000824 | 0.0224 | 0.00197 | 0.00492 | 0.0458 |
| 41 | 0.00917 | 0.211 | 0.00263 | 0.00696 | 0.0187 |
| 41A | 0.12 | 0.417 | | | |
| 41B | 0.0069 | 0.0504 | 0.00101 | 0.000671 | 0.00384 |
| 42 | 0.0322 | 0.524 | | | |
| 43 | 0.018 | 0.417 | 0.0178 | 0.0183 | 0.0436 |
| 44 | 0.00214 | 0.0182 | 0.00381 | 0.0132 | 0.185 |
| 46 | 0.00505 | 0.353 | | 0.0792 | |
| 47 | 0.00721 | 0.262 | 0.0296 | 0.068 | 0.0763 |
| 48 | 0.00714 | 0.0177 | 0.000794 | 0.00114 | 0.0199 |
| 49 | 0.00351 | 0.0263 | 0.0168 | 0.0107 | 0.088 |
| 50 | 0.0569 | 1.115 | 0.441 | | |
| 51 | 0.0264 | 0.176 | 0.0153 | 0.0309 | 0.0607 |
| 53 | 0.0571 | 0.853 | 0.0996 | | |
| 54 | 0.0898 | 0.356 | | | |
| 55 | 0.0116 | 0.147 | 0.0275 | 0.0769 | 0.649 |
| 56 | 0.146 | 1.905 | | | |
| 57 | 0.0458 | 0.35 | 0.0958 | 0.124 | 0.399 |
| 58 | 0.00686 | 0.347 | 0.0495 | | |
| 59 | 0.0403 | 0.0945 | 0.0252 | 0.00525 | 0.0127 |
| 59A | >0.1 | >1 | | | |
| 59B | 0.00165 | 0.00628 | 0.0108 | 0.006 | 0.00554 |
| 60 | 0.0268 | 0.0894 | 0.00785 | 0.0246 | 0.138 |
| 60A | 0.766 | 7.691 | | | |
| 60B | 0.0125 | 0.0629 | 0.00696 | 0.0131 | 0.207 |
| 61 | 0.0235 | 0.284 | 0.0328 | 0.13 | 0.897 |
| 62 | 0.00931 | 0.33 | 0.0134 | 0.0287 | 0.118 |
| 63 | 0.0668 | 0.22 | | | |
| 64 | 0.0454 | 0.329 | 0.0618 | 0.073 | 0.178 |
| 65 | 0.0118 | 0.0998 | 0.00632 | 0.00365 | 0.0159 |
| 67 | 0.00877 | 0.0203 | 0.00372 | 0.00692 | 0.0341 |
| 68 | 0.0144 | 0.131 | 0.00483 | 0.00326 | 0.0078 |
| 69 | 0.0251 | 0.781 | | | 1.069 |
| 70 | 0.00843 | 0.0591 | | | 0.14 |
| 71 | 0.06 | 0.0732 | 0.00931 | 0.205 | 0.723 |
| 72A | 0.0859 | 1.484 | | | |
| 72B | 0.0033 | 0.023 | 0.00151 | 0.00471 | 0.0487 |
| 73 | 0.00302 | 0.037 | 0.000757 | 0.007 | 0.042 |
| 74 | 0.01 | 0.0398 | 0.0124 | 0.00441 | 0.0213 |
| 75 | 0.00926 | 0.0842 | 0.0146 | 0.00564 | 0.0623 |
| 76 | 0.00433 | 0.0648 | 0.0083 | 0.0721 | 0.671 |
| 77 | 0.00511 | 0.0741 | 0.173 | 1.184 | >10 |
| 78 | 0.0088 | 0.0998 | 0.0316 | 0.00404 | 0.0303 |
| 79 | 0.00481 | 0.13 | 0.00666 | 0.00775 | 0.109 |
| 80 | 0.00376 | 0.0694 | 0.00363 | 0.00526 | 0.0241 |
| 81 | 0.00949 | 0.225 | 0.0139 | 0.0162 | 0.153 |
| 82 | 0.1 | >1 | | | |
| 83 | 0.018 | 3.147 | 0.131 | 0.273 | 5.086 |
| 84 | 0.0354 | 0.325 | | | |
| 85 | 0.0691 | 2.213 | | | |
| 86 | 0.0209 | 0.097 | 0.0074 | 0.0603 | 0.528 |
| 87 | 0.0111 | 0.133 | 0.016 | 0.0216 | 0.0612 |
| 88 | 0.0167 | 0.148 | 0.0357 | 0.017 | 0.153 |
| 89 | 0.0205 | 1.943 | >1 | 0.0149 | 0.0966 |
| 90 | 0.00535 | 0.0848 | 0.0124 | 0.0146 | 0.1 |
| 91 | 0.00482 | 0.102 | 0.00854 | 0.0123 | 0.0316 |
| 92 | 0.152 | 3.959 | | | |
| 93 | 0.0932 | 2.18 | | | |
| 94 | 0.00534 | 2.525 | 0.0285 | 0.333 | 1.154 |
| 95 | 0.00328 | 0.388 | >0.2 | 0.0527 | 0.572 |
| 96 | 0.0295 | 0.146 | 0.0342 | 0.205 | 0.394 |
| 97 | 0.00764 | 0.0446 | 0.00522 | 0.0453 | 0.127 |
| 98 | 0.00129 | 0.0201 | 0.0137 | 0.018 | 0.0697 |
| 98A | 0.219 | 4.258 | 1.827 | 0.319 | 1.441 |
| 98B | 0.000826 | 0.0112 | 0.0116 | 0.00603 | 0.0367 |
| 99A | 0.101 | 2.072 | | | |
| 99B | 0.000403 | 0.0135 | | | |
| 100 | 0.00593 | 0.055 | 0.00171 | 0.00568 | 0.033 |
| 101 | 0.00159 | 0.277 | 0.125 | 0.0396 | 0.113 |
| 101B | 0.00115 | 0.138 | 0.139 | 0.0193 | 0.058 |
| 102 | 0.00642 | 0.0742 | 0.0404 | 0.0825 | 1.941 |
| 105 | 0.00157 | 0.0156 | 0.013 | 0.0184 | 0.0633 |
| 106 | 0.000332 | 0.0013 | 0.0086 | 0.0018 | 0.0128 |
| 107A | 0.783 | >10 | | | |
| 107B | 0.00316 | 0.399 | 0.0576 | 0.15 | 0.395 |
| 107 | 0.00411 | 0.524 | 0.14 | 0.107 | 0.282 |
| 108 | 0.0281 | 0.298 | 0.019 | 0.0219 | 0.0636 |
| 109 | 0.0485 | 0.14 | 0.0519 | 0.0283 | 0.0869 |
| 113 | 0.0126 | 0.124 | 0.0159 | 0.00435 | 0.0184 |
| 114 | 0.0123 | 0.12 | 0.00902 | 0.00447 | 0.0187 |
| 115 | 0.0282 | 0.121 | 0.00423 | 0.00423 | 0.00752 |
| 116 | 0.0125 | 0.11 | 0.0108 | 0.0208 | 0.189 |
| 117 | 0.0171 | 0.129 | 0.0103 | 0.0124 | 0.0856 |
| 118 | 0.0192 | 0.165 | 0.064 | 0.0311 | 0.145 |
| 119 | 0.0429 | 0.2 | 0.113 | 0.0816 | 0.362 |
| 120 | 0.0374 | 0.307 | 0.0199 | 0.0281 | 0.18 |
| 121 | 0.0288 | 0.318 | 0.0197 | 0.0112 | 0.067 |
| 126 | 0.00207 | 0.0247 | 0.0208 | 0.000486 | 0.0143 |
| 127 | 0.00118 | 0.0157 | 0.00261 | 0.00182 | 0.024 |
| 128 | >0.1 | 0.519 | | | |
| 130 | 0.00664 | 0.429 | >0.2 | 0.0799 | 0.462 |
| 131 | 0.0091 | 0.0904 | 0.00986 | 0.0134 | 0.055 |
| 132 | 0.0257 | 0.794 | 0.0964 | 0.0332 | 0.111 |
| 133 | 0.00782 | 0.688 | 0.456 | 0.14 | 0.714 |
| 134 | 0.117 | 0.76 | | | |

It is to be understood that, if any prior art publication is referred to herein; such reference does not constitute an admission that the publication forms a part of the common general knowledge in the art in any country.

The disclosures of all publications, patents, patent applications and published patent applications referred to herein by an identifying citation are hereby incorporated herein by reference in their entirety.

Although the foregoing invention has been described in some detail by way of illustration and example for purposes of clarity of understanding, it is apparent to those skilled in the art that certain minor changes and modifications will be practiced. Therefore, the description and Examples should not be construed as limiting the scope of the invention.

1-26. (canceled)

27. A compound of formula (I), a stereoisomer thereof, an atropisomer thereof, a deuterated derivatives thereof, a tautomer thereof, or a pharmaceutically acceptable salt thereof: 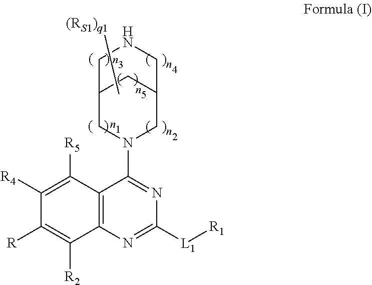
Wherein,
n₁ is selected from 0, 1, 2, 3, 4, 5, or 6; n₂ is selected from 0, 1, 2, 3, 4, 5, or 6; n₃ is selected from 0, 1, 2, 3, 4, 5, or 6; n₄ is selected from 0, 1, 2, 3, 4, 5, or 6; n₅ is selected from 0, 1, 2, 3, 4, 5, or 6;
Each of R_{S1} at each occurrence is independently selected from halogen, —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, —CN, oxo, —N(R₆₁)₂, —OR₆₁, —SR₆₁, —S(═O)R₆₂, —S(═O)₂R₆₂, —C(═O)R₆₂, —C(═O)OR₆₁, —OC(═O)R₆₂, —C(═O)N(R₆₁)₂, —NR₆₁C(═O)R₆₂, —OC(═O)OR₆₁, —NR₆₁C(═O)OR₆, —OC(═O)N(R₆₁)₂, —NR₆₁C(═O)N(R₆₁)₂, —S(═O)OR₆₁, —OS(═O)R₆₂, —S(═O)N(R₆₁)₂, —NR₆₁S(═O)R₆₂, —S(═O)₂OR₆₁, —OS(═O)₂R₆₂, —S(═O)₂N(R₆₁)₂, —NR₆₁S(═O)₂R₆₂, —OS(═O)₂OR₆₁, —NR₆₁S(═O)₂OR₆₁, —OS(═O)₂N(R₆₁)₂, —NR₆₁S(═O)₂N(R₆₁)₂, —P(R₆₁)₂, —P(═O)(R₆₂)₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein, said —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is optionally independently substituted with one or more substituents selected from halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —N(R₆₃)₂, —OR₆₃, —SR₆₃, —S(═O)R₆₄, —S(═O)₂R₆₄, —C(═O)R₆₄, —C(═O)OR₆₃, —OC(═O)R₆₄, —C(═O)N(R₆₃)₂, —NR₆₃C(═O)R₆₄, —OC(═O)OR₆₃, —NR₆₃C(═O)OR₆₃, —OC(═O)N(R₆₃)₂, —NR₆₃C(═O)N(R₆₃)₂, —S(═O)OR₆₃, —OS(═O)R₆₄, —S(═O)N(R₆₃)₂, —NR₆₃S(═O)R₆₄, —S(═O)₂OR₆₃, —OS(═O)₂R₆₄, —S(═O)₂N(R₆₃)₂, —NR₆₃S(═O)₂R₆₄, —OS(═O)₂OR₆₃, —NR₆₃S(═O)₂OR₆₃, —OS(═O)₂N(R₆₃)₂, —NR₆₃S(═O)₂N(R₆₃)₂, —P(R₆₃)₂, —P(═O)(R₆₄)₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;
Optionally, two R_{S1} together with the carbon atom to which they are both attached form a 3-10 membered carbocyclic ring or a 3-10 heterocyclic ring; wherein, said 3-10 membered carbocylic ring or 3-10 heterocyclic ring is optionally substituted with one or more R_{6b};
Optionally, two adjacent R_{S1} together with the carbon atoms to which they are respectively attached form a 3-10 membered carbocyclic ring, a 3-10 membered heterocyclic ring, a 6-10 membered aryl ring or a 5-10 membered heteroaryl ring, wherein, each of rings is independently optionally substituted with one or more R_{6c};
Each of q₁ is independently selected from 0, 1, 2, 3, 4, 5 or 6;
L₁ is a bond, O, S, S(═O), S(═O)₂ or NR₆ₐ;
R₁ is selected from 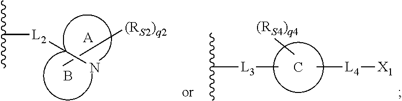
L₂ is selected from a bond or C₁₋₁₀ alkylene optionally substituted with one or more R_{6d};
L₃ is selected from a bond or C₁₋₁₀ alkylene optionally substituted with one or more R₆ₑ;
L₄ is selected from a bond or C₁₋₁₀ alkylene optionally substituted with one or more R_{6f};
Ring A or ring B is independently selected from a 3-10 membered heterocyclic ring which is optionally further contains 1, 2, or 3 heteroatoms selected from N, O or S;
Ring C is selected from a 3-10 membered carbocyclic ring or a 3-10 membered heterocyclic ring; wherein the moiety of -L₃- and -L₄-X₁ are attached to the same atom or different atoms of the ring C;
X₁ is selected from —N(R₆₅)₂, —OR₆₅, —SR₆₅, 3-10 membered heterocyclyl, or 5-10 membered heteroaryl, wherein said 3-10 membered heterocyclyl or 5-10 membered heteroaryl is optionally independently substituted with one or more R_{S3};
Each of (R_{S2} and R_{S3}) at each occurrence is independently selected from halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, oxo, —N(R₆₆)₂, —OR₆₆, —SR₆₆, —S(═O)R₆₇, —S(═O)₂R₆₇, —C(═O)R₆₇, —C(═O)OR₆₆, —OC(═O)R₆₇, —C(═O)N(R₆₆)₂, —NR₆₆C(═O)R₆₇, —OC(═O)OR₆₆, —NR₆₆C(═O)OR₆₆, —NR₆₆C(═S)OR₆₆, —OC(═O)N(R₆₆)₂, —NR₆₆C(═O)N(R₆₆)₂, —S(═O)OR₆₆, —OS(═O)R₆₇, —S(═O)N(R₆₆)₂, —NR₆₆S(═O)R₆₇, —S(═O)₂OR₆₆, —OS(═O)₂R₆₇, —S(═O)₂N(R₆₆)₂, —NR₆₆S(═O)₂R₆₇, —OS(═O)₂OR₆₆, —NR₆₆S(═O)₂OR₆₆, —OS(═O)₂N(R₆₆)₂, —NR₆₆S(═O)₂N(R₆₆)₂, —P(R₆₆)₂, —P(═O)(R₆₇)₂, 3-8 membered cycloalkyl, 3-8 membered cycloalkenyl, 3-8 membered cycloalkynyl, 4-8 membered heterocyclyl, 6-10 membered aryl, 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-8 membered cycloalkyl, 3-8 membered cycloalkenyl, 3-8 membered cycloalkynyl, 3-8 membered heterocyclyl, 6-10 membered aryl or 5-10 membered heteroaryl is optionally independently substituted with one or more substituents selected from halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —N(R₆₈)₂, —OR₆₈, —SR₆₈, —S(═O)R₆₉, —S(═O)₂R₆₉, —C(═O)R₆₉, —C(═O)OR₈, —OC(═O)R₆₉, —C(═O)N(R₆₈)₂, —NR₆₈C(═O)R₆₉, —OC(═O)OR₆₈, —NR₆₈C(═O)OR₆₈, —NR₆₈C(═S)OR₆₈, —OC(═O)N(R₆₈)₂, —NR₆₈C(═O)N(R₆₈)₂, —S(═O)OR₆₈, —OS(═O)R₆₉, —S(═O)N(R₆₈)₂, —NR₆₈S(═O)R₆₉, —S(═O)₂OR₆₈, —OS(═O)₂R₆₉, —S(═O)₂N(R₆₈)₂, —NR₆₈S(═O)₂R₆₉, —OS(═O)₂OR₆₈, —NR₆₈S(═O)₂OR₆₈, —OS(═O)₂N(R₆₈)₂, —NR₆₈S(═O)₂N(R₆₈)₂, —P(R₆₈)₂, —P(═O)(R₆₉)₂, 3-6 membered cycloalkyl, 3-6 membered cycloalkenyl, 3-6 membered cycloalkynyl, 3-6 membered heterocyclyl, 6-10 membered aryl or 5-10 membered heteroaryl;
Optionally, two R_{S2} together with the carbon atom to which they are both attached or two R_{S3} together with the carbon atom to which they are both attached form a 3-10 membered carbocyclic ring or a 3-10 heterocyclic ring; wherein, sais 3-10 membered carbocylic ring or 3-10 heterocyclic ring is optionally substituted with one or more R₆ₕ;
Optionally, two adjacent R_{S2} together with the carbon atoms to which they are respectively attached or two adjacent R_{S3} together with the carbon atoms to which they are respectively attached form a 3-10 membered carbocyclic ring, a 3-10 membered heterocyclic ring, a 6-10 membered aryl ring or a 5-10 membered heteroaryl ring, wherein, each of rings is independently optionally substituted with one or more R₆ᵢ;
Optionally, two nonadjacent R_{S2} or two nonadjacent R_{S3} are connected together to form a bridge containing 0, 1, 2, 3, 4, 5 or 6 carbon atoms, wherein, each of the carbon atoms in the bridge is optionally replaced by 1 or 2 heteroatoms selected from N, O, S, S═O or S(═O)₂; the hydrogen on the each of carbon atoms or N atoms is optionally independently substituted with R₆ⱼ;
q₂ is selected from 0, 1, 2, 3, 4, 5 or 6;
Each of R_{S4} at each occurrence is independently selected from halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, oxo, —N(R₇₁)₂, —OR₇₁, —SR₇₁, —S(═O)R₇₂, —S(═O)₂R₇₁, —C(═O)R₇₂, —C(═O)OR₇₁, —OC(═O)R₇₂, —C(═O)N(R₇₁)₂, —NR₇₁C(═O)R₇₂, —OC(═O)OR₇₁, —NR₇₁C(═O)OR₇₁, —OC(═O)N(R₇₁)₂, —NR₇₁C(═O)N(R₇₁)₂, —S(═O)OR₇₁, —OS(═O)R₇₂, —S(═O)N(R₇₁)₂, —NR₇₁S(═O)R₇₂, —S(═O)₂OR₇₁, —OS(═O)₂R₇₂, —S(═O)₂N(R₇₁)₂, —NR₇₁S(═O)₂R₇₂, —OS(═O)₂OR₇₁, —NR₇₁S(═O)₂OR₇₂, —OS(═O)₂N(R₇₁)₂, —NR₇₁S(═O)₂N(R₇₁)₂, —P(R₇₁)₂, —P(═O)(R₇₂)₂, 3-6 membered cycloalkyl, 3-6 membered cycloalkenyl, 3-6 membered cycloalkynyl, 3-6 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-6 membered cycloalkyl, 3-6 membered cycloalkenyl, 3-6 membered cycloalkynyl, 3-6 membered heterocyclyl, 6-10 membered aryl or 5-10 membered heteroaryl is optionally independently substituted with one or more substituents selected from halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —N(R₇₃)₂, —OR₇₃, —SR₇₃, —S(═O)R₇₄, —S(═O)₂R₇₃, —C(═O)R₇₄, —C(═O)OR₇₃, —OC(═O)R₇₄, —C(═O)N(R₇₃)₂, —NR₇₃C(═O)R₇₄, —OC(═O)OR₇₃, —NR₇₃C(═O)OR₇₃, —OC(═O)N(R₇₃)₂, —NR₇₃C(═O)N(R₇₃)₂, —S(═O)OR₇₃, —OS(═O)R₇₄, —S(═O)N(R₇₃)₂, —NR₇₃S(═O)R₇₄, —S(═O)₂OR₇₃, —OS(═O)₂R₇₄, —S(═O)₂N(R₇₃)₂, —NR₇₃S(═O)₂R₇₄, —OS(═O)₂OR₇₃, —NR₇₃S(═O)₂OR₇₄, —OS(═O)₂N(R₇₃)₂, —NR₇₃S(═O)₂N(R₇₃)₂, —P(R₇₃)₂, —P(═O)(R₇₄)₂, 3-6 membered cycloalkyl, 3-6 membered cycloalkenyl, 3-6 membered cycloalkynyl, 3-6 membered heterocyclyl, 6-10 membered aryl or 5-10 membered heteroaryl;
q₄ is selected from 0, 1, 2, 3, 4, 5 or 6;
R₃ is selected from 6-10 membered aryl or 5-10 membered heteroaryl, wherein said 6-10 membered aryl or 5-10 membered heteroaryl is optionally independently substituted with one or more R₃₁;
R₃₁ at each occurrence is independently selected from halogen, —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, —CN, oxo, —N(R₇₅)₂, —OR₇₅, —SR₇₅, —S(═O)R₇₆, —S(═O)₂R₇₆, —C(═O)R₇₆, —C(═O)OR₇₅, —OC(═O)R₇₆, —C(═O)N(R₇₅)₂, —NR₇₅C(═O)R₇₆, —OC(═O)OR₇₅, —NR₇₅C(═O)OR₇₅, —OC(═O)N(R₇₅)₂, —NR₇₅C(═O)N(R₇₅)₂, —S(═O)OR₇₅, —OS(═O)R₇₆, —S(═O)N(R₇₅)₂, —NR₇₅S(═O)R₇₆, —S(═O)₂OR₇₅, —OS(═O)₂R₇₆, —S(═O)₂N(R₇₅)₂, —NR₇₅S(═O)₂R₇₆, —OS(═O)₂OR₇₅, —NR₇₅S(═O)₂OR₇₅, —OS(═O)₂N(R₇₅)₂, —NR₇₅S(═O)₂N(R₇₅)₂, —P(R₇₅)₂, —P(═O)(R₇₅)₂, 3-10 membered cycloalkyl, 3-10 membered cycloalkenyl, 3-10 membered cycloalkynyl, 3-10 membered heterocyclyl, 6-10 membered aryl or 5-10 membered heteroaryl, wherein said —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is optionally independently substituted with one or more substituents selected from halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —N(R₇₇)₂, —OR₇₇, —SR₇₇, —S(═O)R₇₈, —S(═O)₂R₇₈, —C(═O)R₇₈, —C(═O)OR₇₇, —OC(═O)R₇₈, —C(═O)N(R₇₇)₂, —NR₇₇C(═O)R₇₈, —OC(═O)OR₇₇, —NR₇₇C(═O)OR₇₇, —OC(═O)N(R₇₇)₂, —NR₇₇C(═O)N(R₇₇)₂, —S(═O)OR₇₇, —OS(═O)R₇₈, —S(═O)N(R₇₇)₂, —NR₇₇S(═O)R₇₈, —S(═O)₂OR₇₇, —OS(═O)₂R₇₈, —S(═O)₂N(R₇₇)₂, —NR₇₇S(═O)₂R₇₈, —OS(═O)₂OR₇₇, —NR₇₇S(═O)₂OR₇₇, —OS(═O)₂N(R₇₇)₂, —NR₇₇S(═O)₂N(R₇₇)₂, —P(R₇₇)₂, —P(═O)(R₇₈)₂, 3-6 membered cycloalkyl, 3-6 membered cycloalkenyl, 3-6 membered cycloalkynyl, 3-6 membered heterocyclyl, 6-10 membered aryl or 5-10 membered heteroaryl;
Each of (R₂, R₄ and R₅) is independently selected from hydrogen, halogen, —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, —CN, oxo, —N(R₈₁)₂, —OR₈₁, —SR₈₁, —S(═O)R₈₂, —S(═O)₂R₈₂, —C(═O)R₈₂, —C(═O)OR₈₁, —OC(═O)R₈₂, —C(═O)N(R₈₁)₂, —NR₈₁C(═O)R₈₂, —OC(═O)OR₈₁, —NR₈₁C(═O)OR₈₁, —OC(═O)N(R₈₁)₂, —NR₈₁C(═O)N(R₈₁)₂, —S(═O)OR₈₁, —OS(═O)R₈₂, —S(═O)N(R₈₁)₂, —NR₈₁S(═O)R₈₂, —S(═O)₂OR₈₁, —OS(═O)₂R₈₂, —S(═O)₂N(R₈₁)₂, —NR₈₁S(═O)₂R₈₂, —OS(═O)₂OR₈₁, —NR₈₁S(═O)₂OR₈₁, —OS(═O)₂N(R₈₁)₂, —NR₈₁S(═O)₂N(R₈₁)₂, —P(R₈₁)₂, —P(═O)(R₈₂)₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is optionally independently substituted with one or more substituents selected from halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —N(R₈₃)₂, —OR₈₃, —SR₈₃, —S(═O)R₈₄, —S(═O)₂R₈₄, —C(═O)R₈₄, —C(═O)OR₈₃, —OC(═O)R₈₃, —C(═O)N(R₈₃)₂, —NR₈₃C(═O)R₈₄, —OC(═O)OR₈₃, —NR₈₃C(═O)OR₈₃, —OC(═O)N(R₈₃)₂, —NR₈₃C(═O)N(R₈₄)₂, —S(═O)OR₈₃, —OS(═O)R₈₄, —S(═O)N(R₈₃)₂, —NR₈₃S(═O)R₈₄, —S(═O)₂OR₈₃, —OS(═O)₂R₈₄, —S(═O)₂N(R₈₃)₂, —NR₈₃S(═O)₂R₈₄, —OS(═O)₂OR₈₃, —NR₈₃S(═O)₂OR₈₃, —OS(═O)₂N(R₈₃)₂, —NR₈₃S(═O)₂N(R₈₃)₂, —P(R₈₃)₂, —P(═O)(R₈₄)₂, 3-6 membered cycloalkyl, 3-6 membered cycloalkenyl, 3-6 membered cycloalkynyl, 3-6 membered heterocyclyl, 6-10 membered aryl or 5-10 membered heteroaryl;
Each of (R₆ₐ, R₆₁, R₆₃, R₆₅, R₆₆, R₆₈, R₇₁, R₇₃, R₇₅, R₇₇, R₈₁ and R₈₃) at each occurrence is independently selected from hydrogen, halogen, —C₁₋₁₀alkyl, haloC₁₋₁₀alkyl, —C₂₋₁₀alkenyl, —C₂₋₁₀alkynyl, —S(═O)Rₐ, —S(═O)₂Rₐ, —C(═O)Rₐ, —C(═O)ORₐ, —C(═O)N(Rₐ)₂, —S(═O)ORₐ, —S(═O)N(Rₐ)₂, —S(═O)₂ORₐ, —S(═O)₂N(Rₐ)₂, —P(═O)(Rₐ)₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₁₀alkyl, haloC₁₋₁₀alkyl, —C₂₋₁₀alkenyl, —C₂₋₁₀alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is optionally independently substituted with one or more substituents selected from halogen, —C₁₋₆alkyl, haloC₁₋₆alkyl, haloC₁₋₆alkoxy, —C₂₋₆alkenyl, —C₂₋₆alkynyl, —CN, —N(R_{c})₂, —OR_{c}, —SR_{c}, —S(═O)R_{d}, —S(═O)₂R_{d}, —C(═O)R_{d}, —C(═O)OR_{c}, —OC(═O)R_{d}, —C(═O)N(R_{c})₂, —NR_{c}C(═O)R_{d}, —OC(═O)OR_{c}, —NR_{c}C(═O)OR_{d}, —OC(═O)N(R_{c})₂, —NR_{c}C(═O)N(R_{c})₂, —S(═O)OR_{c}, —OS(═O)R_{d}, —S(═O)N(R_{c})₂, —NR_{c}S(═O)R_{d}, —S(═O)₂OR_{c}, —OS(═O)₂R_{d}, —S(═O)₂N(R_{c})₂, —NR_{c}S(═O)₂R_{d}, —OS(═O)₂OR_{c}, —NR_{c}S(═O)₂OR_{c}, —OS(═O)₂NR_{c}, —NR_{c}S(═O)₂N(R_{c})₂, —P(R_{c})₂, —P(═O)(R_{d})₂, 3-6 membered cycloalkyl, 3-6 membered cycloalkenyl, 3-6 membered cycloalkynyl, 3-6 membered heterocyclyl, 6-10 membered aryl or 5-10 membered heteroaryl;
Optionally, each of (two R₆₁, two R₆₃, two R₆₅, two R₆₆, two R₆₈, two R₇₁, two R₇₃, two R₇₅, two R₇₇, two R₈₁, and two R₈₃) independently together with the nitrogen atom to which they are both attached forms a 3-20 membered heterocyclic ring or a 5-10 membered heteroaryl ring, wherein, said 3-20 membered heterocyclic ring or 5-10 membered heteroaryl ring is optionally independently substituted with one or more R₆ₖ;
Each of (R₆₂, R₆₄, R₆₇, R₆₉, R₇₂, R₇₄, R₇₆, R₇₈, R₈₂ and R₈₄) at each occurrence is independently selected from hydrogen, —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, —N(R_{b})₂, —OR_{b}, —SR_{b}, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₁₀alkyl, haloC₁₋₁₀alkyl, haloC₁₋₁₀alkoxy, —C₂₋₁₀alkenyl, —C₂₋₁₀alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is optionally independently substituted with one or more substituents selected from halogen, —C₁₋₆alkyl, haloC₁₋₆alkyl, haloC₁₋₆alkoxy, —C₂₋₆alkenyl, —C₂₋₆alkynyl, —CN, —N(R_{c})₂, —OR, —SR, —S(═O)R_{d}, —S(═O)₂R_{d}, —C(═O)R_{d}, —C(═O)OR_{c}, —OC(═O)R_{d}, —C(═O)N(R_{c})₂, —NR_{c}C(═O)R_{d}, —OC(═O)OR_{c}, —NR_{c}C(═O)OR_{d}, —OC(═O)N(R_{c})₂, —NR_{c}C(═O)N(R_{c})₂, —S(═O)OR_{c}, —OS(═O)R_{d}, —S(═O)N(R_{c})₂, —NR_{c}S(═O)R_{d}, —S(═O)₂OR_{c}, —OS(═O)₂R_{d}, —S(═O)₂N(R_{c})₂, —NR_{c}S(═O)₂R_{d}, —OS(═O)₂OR_{c}, —NR_{c}S(═O)₂OR_{c}, —OS(═O)₂NR_{c}, —NR_{c}S(═O)₂N(R_{c})₂, —P(R_{c})₂, —P(═O)(R_{d})₂, 3-6 membered cycloalkyl, 3-6 membered cycloalkenyl, 3-6 membered cycloalkynyl, 3-10 membered heterocyclyl, 6-10 membered aryl or 5-10 membered heteroaryl;
Each of (Rₐ, R_{b}, R_{c} and R_{d}) at each occurrence is independently selected from hydrogen, —C₁₋₆alkyl, haloC₁₋₆alkyl, haloC₁₋₆alkoxy, —C₂₋₆alkenyl, —C₂₋₆alkynyl, 3-6 membered cycloalkyl, 3-6 membered cycloalkenyl, 3-6 membered cycloalkynyl, 3-6 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, —C₂₋₆alkynyl, 3-6 membered cycloalkyl, 3-6 membered cycloalkenyl, 3-6 membered cycloalkynyl, 3-6 membered heterocyclyl, 6-10 membered aryl or 5-10 membered heteroaryl is optionally independently substituted with one or more R₆₁;
Optionally, each of (two Rₐ, two R_{b} and two R_{c}) independently together with the atom to which they are both attached forms a 3-6 membered heterocyclic ring, wherein said 3-6 membered heterocyclic ring is independently optionally substituted with one or more R₆ₘ;
Each of (R_{6b}, R_{6c}, R_{6d}, R₆ₑ, R_{6f}, R₆ₕ, R₆ᵢ, R₆ⱼ, R₆ₖ, R₆ₗ and R₆ₘ) at each occurrence is independently selected from halogen, —C₁₋₆alkyl, haloC₁₋₆alkyl, haloC₁₋₆alkoxy, —C₂₋₆alkenyl, —C₂₋₆alkynyl, —CN, oxo, —NH₂, —NH(C₁₋₆alkyl), —N(C₁₋₆alkyl)₂, —OH, —O(C₁₋₆alkyl), —SH, —S(C₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —OC(═O)O(C₁₋₆alkyl), —NHC(═O)(OC₁₋₆alkyl), —N(C₁₋₆alkyl)C(═O)(OC₁₋₆alkyl), —OC(═O)NH(C₁₋₆alkyl), —OC(═O)N(C₁₋₆alkyl)₂, —NHC(═O)NH₂, —NHC(═O)NH(C₁₋₆alkyl), —NHC(═O)N(C₁₋₆alkyl)₂, —N(C₁₋₆alkyl)C(═O)NH₂, —N(C₁₋₆alkyl)C(═O)NH(C₁₋₆alkyl), —N(C₁₋₆alkyl)C(═O)N(C₁₋₆alkyl)₂, —S(═O)(OC₁₋₆alkyl), —OS(═O)(C₁₋₆alkyl), —S(═O)NH₂, —S(═O)NH(C₁₋₆alkyl), —S(═O)N(C₁₋₆alkyl)₂, —NHS(═O)(C₁₋₆alkyl), —N(C₁₋₆alkyl)S(═O)(C₁₋₆alkyl), —S(═O)₂(OC₁₋₆alkyl), —OS(═O)₂(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), —OS(═O)₂O(C₁₋₆alkyl), —NHS(═O)₂O(C₁₋₆alkyl), —N(C₁₋₆alkyl)S(═O)₂O(C₁₋₆alkyl), —OS(═O)₂NH₂, —OS(═O)₂NH(C₁₋₆alkyl), —OS(═O)₂N(C₁₋₆alkyl)₂, —NHS(═O)₂NH₂, —NHS(═O)₂NH(C₁₋₆alkyl), —NHS(═O)₂N(C₁₋₆alkyl)₂, —N(C₁₋₆alkyl)S(═O)₂NH₂, —N(C₁₋₆alkyl)S(═O)₂NH(C₁₋₆alkyl), —N(C₁₋₆alkyl)S(═O)₂N(C₁₋₆alkyl)₂, —PH(C₁₋₆alkyl), —P(C₁₋₆alkyl)₂, —P(═O)H(C₁₋₆alkyl), —P(═O)(C₁₋₆alkyl)₂, 3-6 membered cycloalkyl, 3-6 membered cycloalkenyl, 3-6 membered cycloalkynyl, 3-6 membered heterocyclyl, 6-10 membered aryl or 5-10 membered heteroaryl; wherein, said —C₁₋₆alkyl, haloC₁₋₆alkyl, haloC₁₋₆alkoxy, —C₂₋₆alkenyl, —C₂₋₆alkynyl, 3-6 membered cycloalkyl, 3-6 membered cycloalkenyl, 3-6 membered cycloalkynyl, 3-6 membered heterocyclyl, 6-10 membered aryl or 5-10 membered heteroaryl is optionally substituted with one or more substituents selected from halogen, —C₁₋₃alkyl, haloC₁₋₃alkyl, haloC₁₋₃alkoxy, —C₂₋₃alkenyl, —C₂₋₃alkynyl, —CN, —NH₂, —NH(C₁₋₃alkyl), —N(C₁₋₃alkyl)₂, —OH, —O(C₁₋₃alkyl), —SH, —S(C₁₋₃alkyl), —S(═O)(C₁₋₃alkyl), —S(═O)₂(C₁₋₃alkyl), —C(═O)(C₁₋₃alkyl), —C(═O)OH, —C(═O)(OC₁₋₃alkyl), —OC(═O)(C₁₋₃alkyl), —C(═O)NH₂, —C(═O)NH(C₁₋₃alkyl), —C(═O)N(C₁₋₃alkyl)₂, —NHC(═O)(C₁₋₃alkyl), —N(C₁₋₃alkyl)C(═O)(C₁₋₃alkyl), —OC(═O)O(C₁₋₃alkyl), —NHC(═O)(OC₁₋₃alkyl), —N(C₁₋₃alkyl)C(═O)(OC₁₋₃alkyl), —OC(═O)NH(C₁₋₃alkyl), —OC(═O)N(C₁₋₃alkyl)₂, —NHC(═O)NH₂, —NHC(═O)NH(C₁₋₃alkyl), —NHC(═O)N(C₁₋₃alkyl)₂, —N(C₁₋₃alkyl)C(═O)NH₂, —N(C₁₋₃alkyl)C(═O)NH(C₁₋₃alkyl), —N(C₁₋₃alkyl)C(═O)N(C₁₋₃alkyl)₂, —S(═O)(OC₁₋₃alkyl), —OS(═O)(C₁₋₃alkyl), —S(═O)NH₂, —S(═O)NH(C₁₋₃alkyl), —S(═O)N(C₁₋₃alkyl)₂, —NHS(═O)(C₁₋₃alkyl), —N(C₁₋₃alkyl)S(═O)(C₁₋₃alkyl), —S(═O)₂(OC₁₋₃alkyl), —OS(═O)₂(C₁₋₃alkyl), —S(═O)₂NH₂, —S(═O)₂NH(C₁₋₃alkyl), —S(═O)₂N(C₁₋₃alkyl)₂, —NHS(═O)₂(C₁₋₃alkyl), —N(C₁₋₃alkyl)S(═O)₂(C₁₋₃alkyl), —OS(═O)₂O(C₁₋₃alkyl), —NHS(═O)₂O(C₁₋₃alkyl), —N(C₁₋₃alkyl)S(═O)₂O(C₁₋₃alkyl), —OS(═O)₂NH₂, —OS(═O)₂NH(C₁₋₃alkyl), —OS(═O)₂N(C₁₋₃alkyl)₂, —NHS(═O)₂NH₂, —NHS(═O)₂NH(C₁₋₃alkyl), —NHS(═O)₂N(C₁₋₃alkyl)₂, —N(C₁₋₃alkyl)S(═O)₂NH₂, —N(C₁₋₃alkyl)S(═O)₂NH(C₁₋₃alkyl), —N(C₁₋₃alkyl)S(═O)₂N(C₁₋₃alkyl)₂, —PH(C₁₋₃alkyl), —P(C₁₋₃alkyl)₂, —P(═O)H(C₁₋₃alkyl), —P(═O)(C₁₋₃alkyl)₂, 3-6 membered cycloalkyl, 3-6 membered cycloalkenyl, 3-6 membered cycloalkynyl, 3-6 membered heterocyclyl, 6 membered aryl or 5-6 membered heteroaryl;
Each of (heterocyclyl and heteroaryl) at each occurrence is independently contain 1, 2, 3 or 4 heteroatoms selected from N, O, S, S(═O) or S(═O)₂.

28. The compound of claim 27, wherein,
L₁ is a bond, O, S, S(═O), S(═O)₂ or NR₆a;
R₁ is selected from 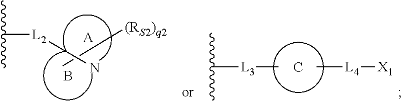
L₂ is selected from a bond or C₁₋₃alkylene optionally substituted with one or more R_{6d};
L₃ is selected from a bond or C₁₋₃alkylene optionally substituted with one or more R₆ₑ;
L₄ is selected from a bond or C₁₋₃ alkylene optionally substituted with one or more R_{6f};
Ring A or ring B is a 5 membered heterocyclic ring which is optionally further contains 1, 2, or 3 heteroatoms selected from N, O or S;
Ring C is selected from a 3-6 membered carbocyclic ring or a 3-6 heterocyclic ring; wherein the moiety of -L₃- and -L₄-X₁ are attached to the same atom or different atoms of the ring C;
X₁ is selected from —N(R₆₅)₂, 5-6 membered heterocyclyl, or 5-10 membered heteroaryl, wherein said 5-6 membered heterocyclyl or 5-10 membered heteroaryl is optionally independently substituted with 1, 2, 3, 4, 5, or 6 R_{S3}; and
R_{S2}, R_{S3}, q₂, R_{6d}, R₆ₑ, R_{6f}, and R₆₅ have the same definition as in 1.

29. The compound of claim 27, wherein,
-L₁-R₁ is selected from 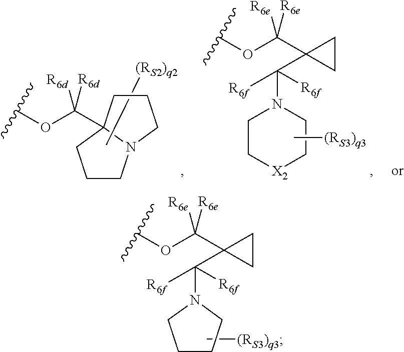
wherein q₃ is selected from 0, 1, 2, 3, 4, 5, or 6.

30. The compound of claim 27, wherein,
the compound is selected from any one of formula (II-1) to (II-6): 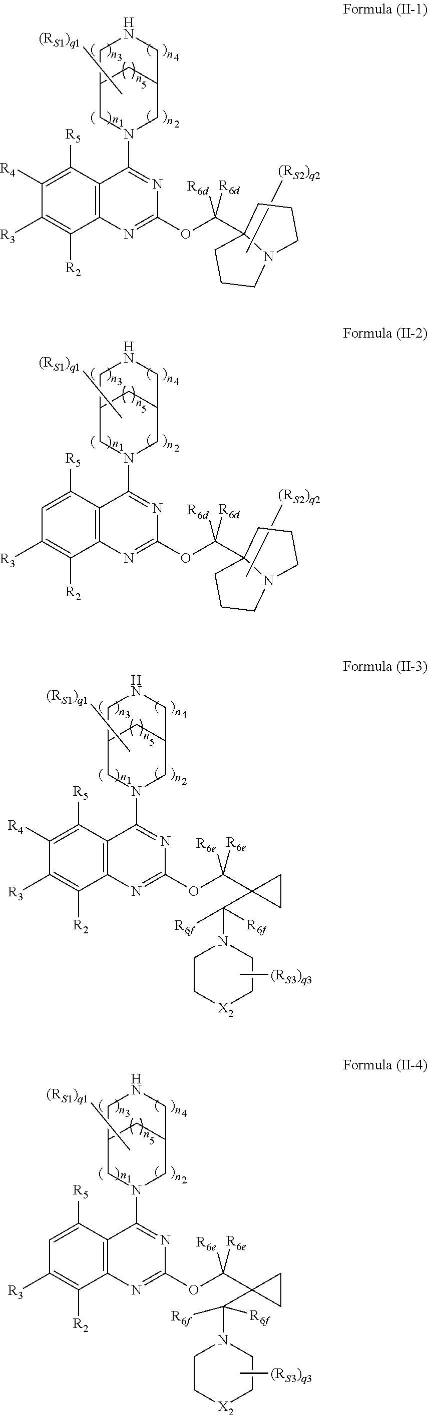 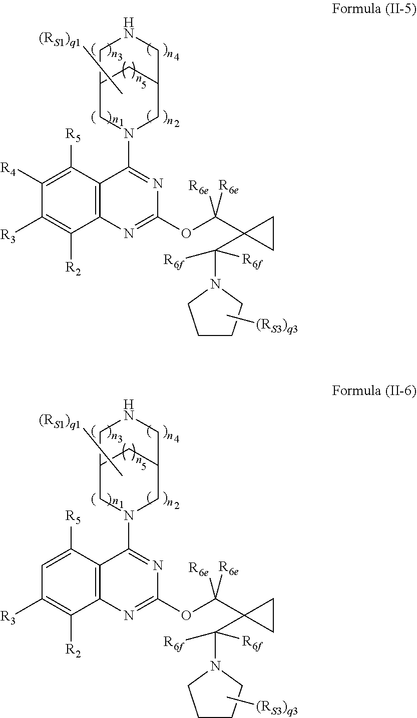
Wherein,
R₄ is selected from 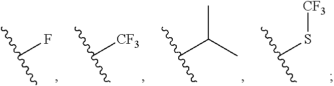
X₂ is selected from CH₂, NH, O, S, S═O, or O═S═O; and R₂, R₃, R₅, R_{S1}, R_{S2}, R_{S3}, R_{6d}, R₆ₑ, R_{6f}, n₁, n₂, n₃, n₄, n₅, q₁, q₂, and q₃ have the same definitions as in claim 27.

31. The compound of claim 27, wherein,
n₃, n₄, n₅ or n₆ is independently selected from 0 or 1, and n₇ is selected from 1 or 2; provided that satisfies one of the following conditions:
n₃ and n₄ are 0, and n₅ and n₆ are 1;
n₃ and n₄ are 1, and n₅ and n₆ are 0;
n₃ and n₅ are 0, and n₄ and n₆ are 1; or
n₃ and n₅ are 1, and n₄ and n₆ are 0.

32. The compound of claim 27, wherein,
the moiety of 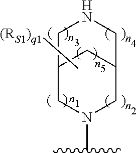
is selected from

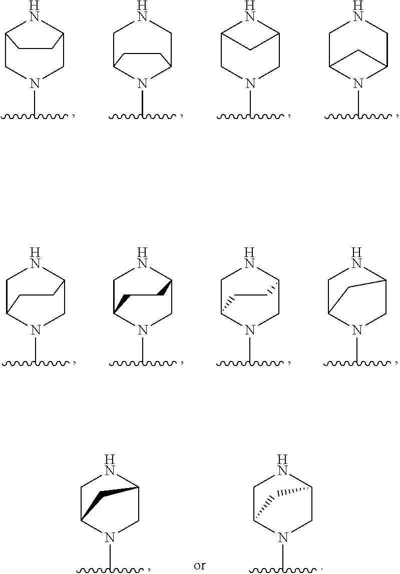

33. The compound of claim 27, wherein,
the compound is selected from any one of formula (III-1) to (III-60):

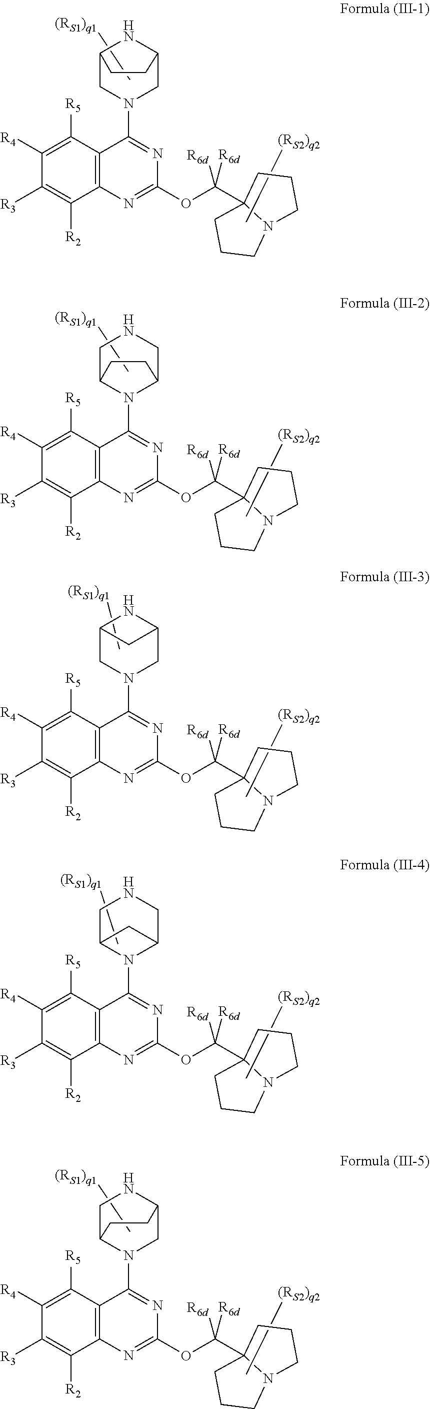

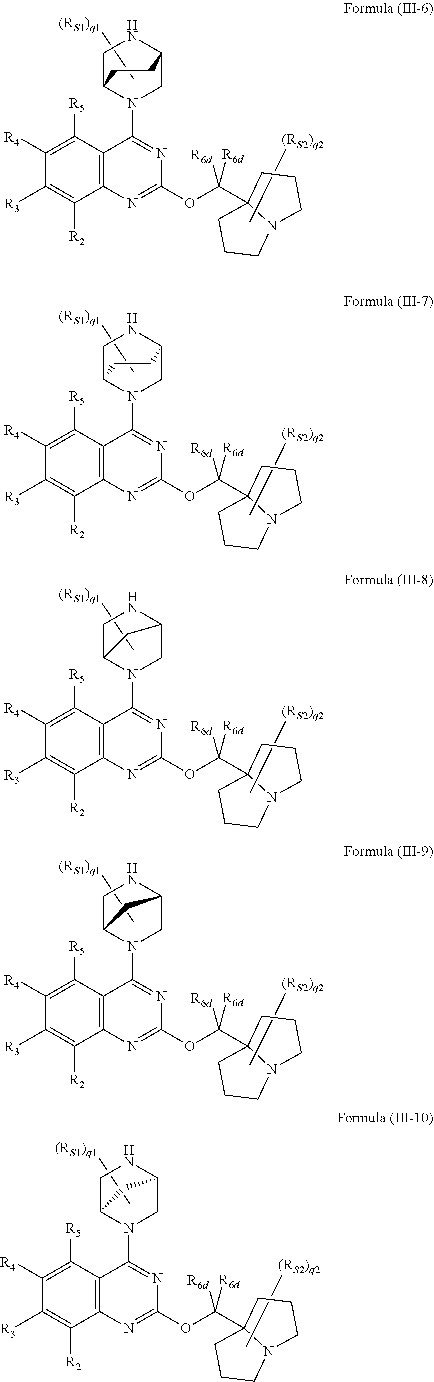

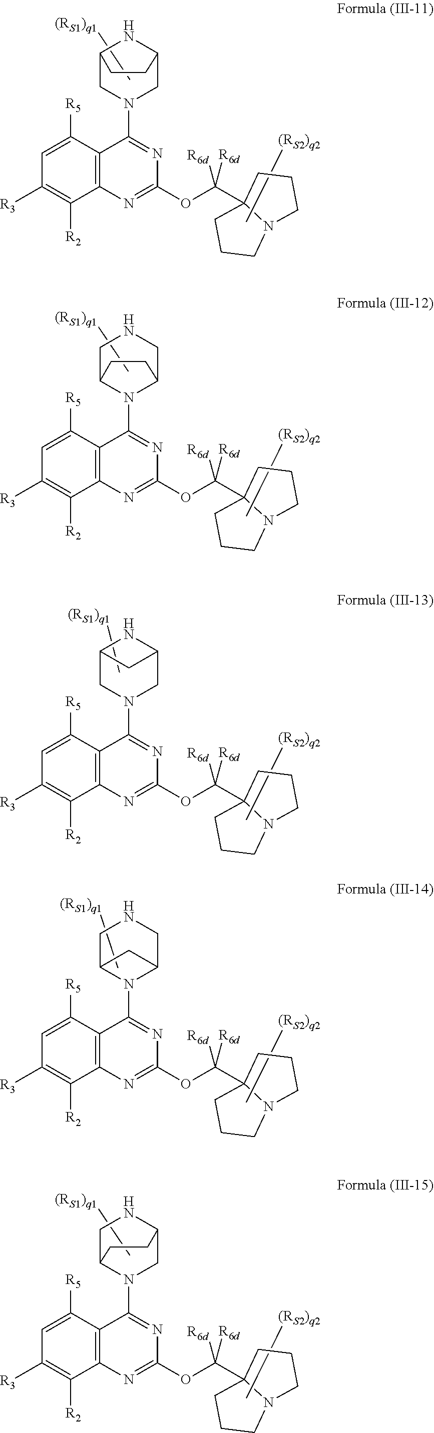

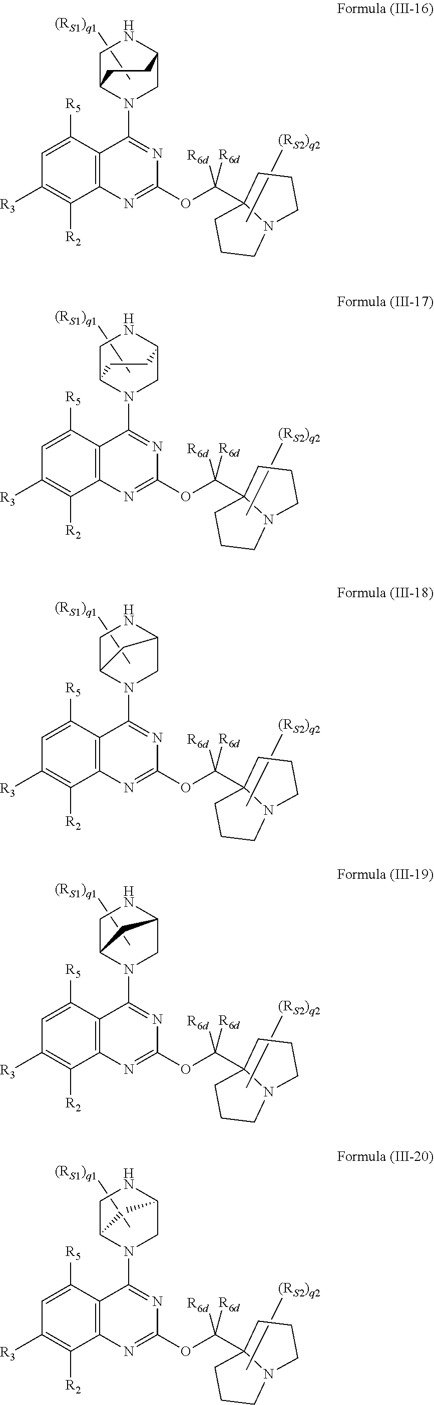

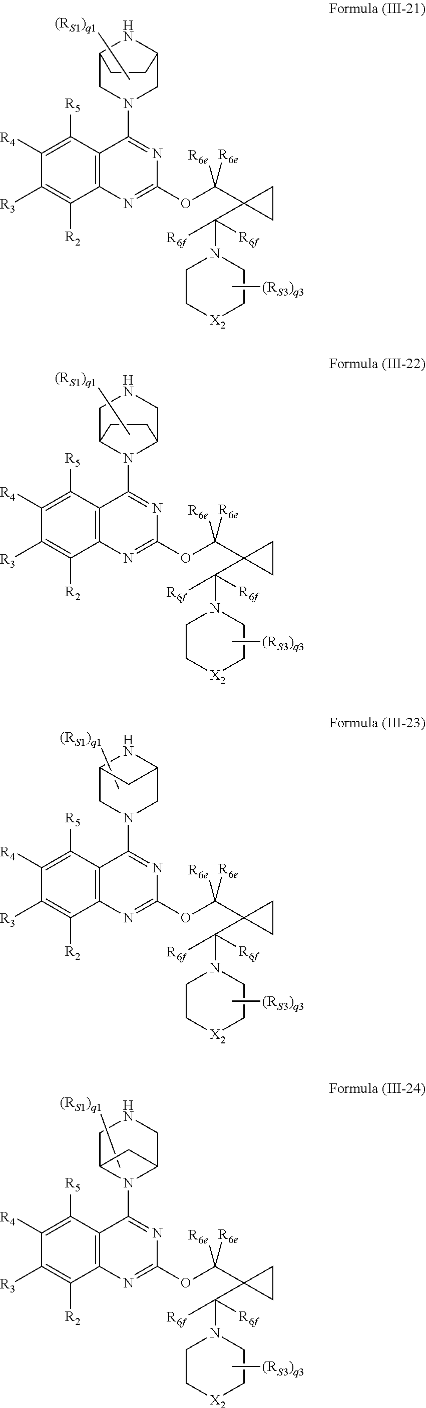

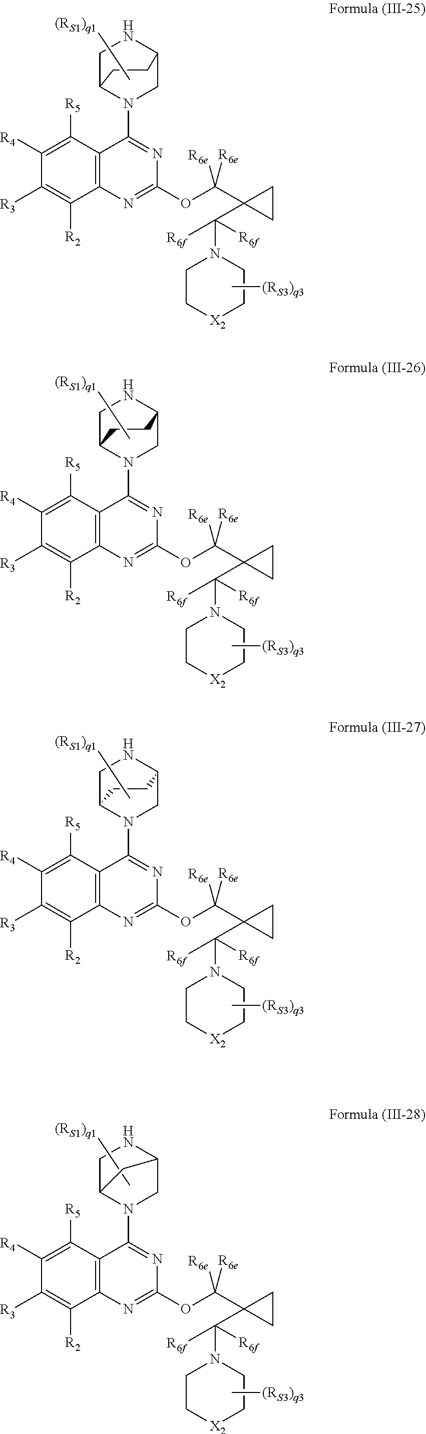

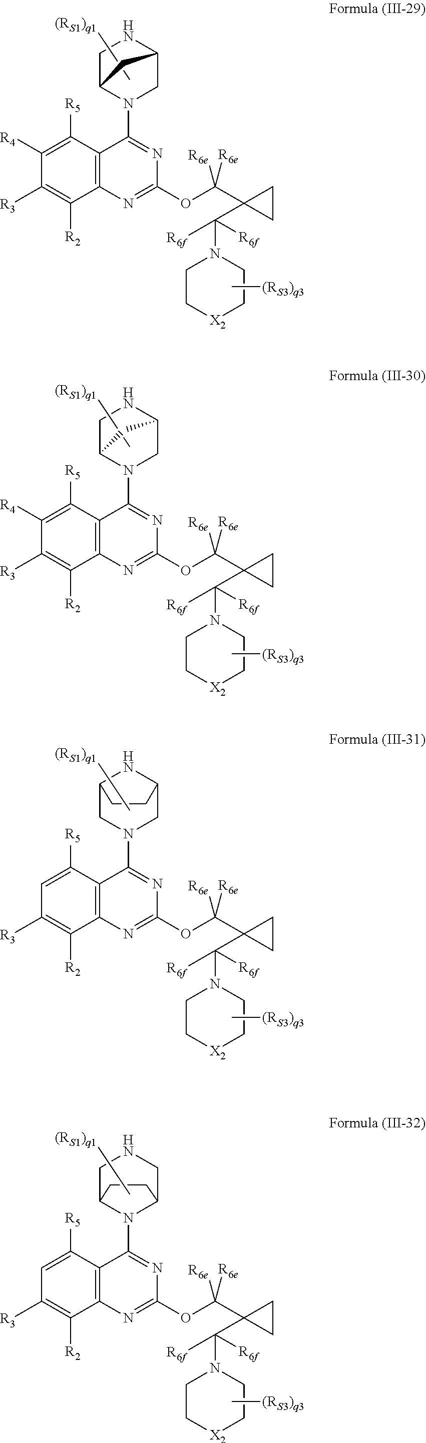

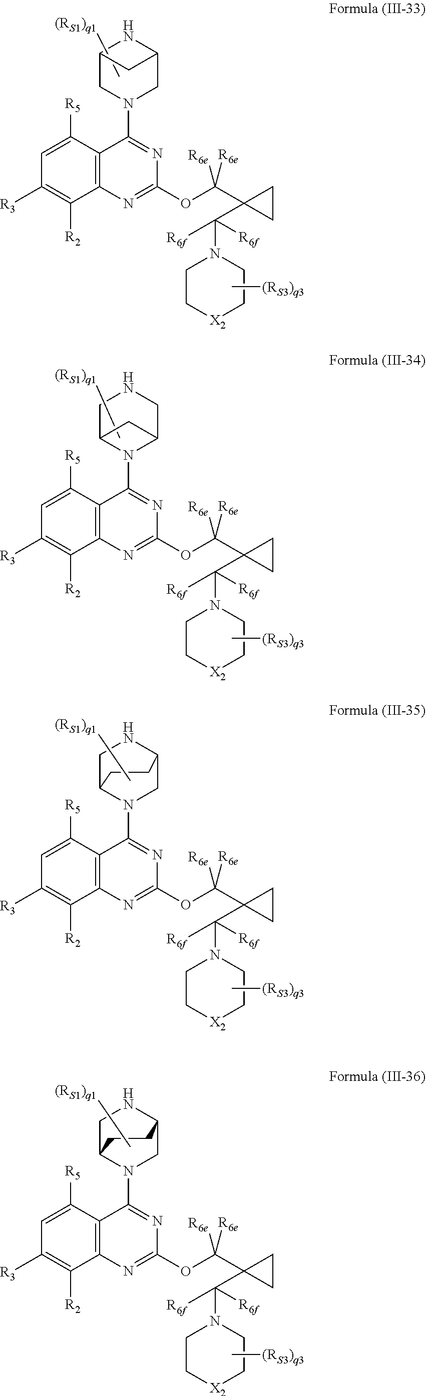

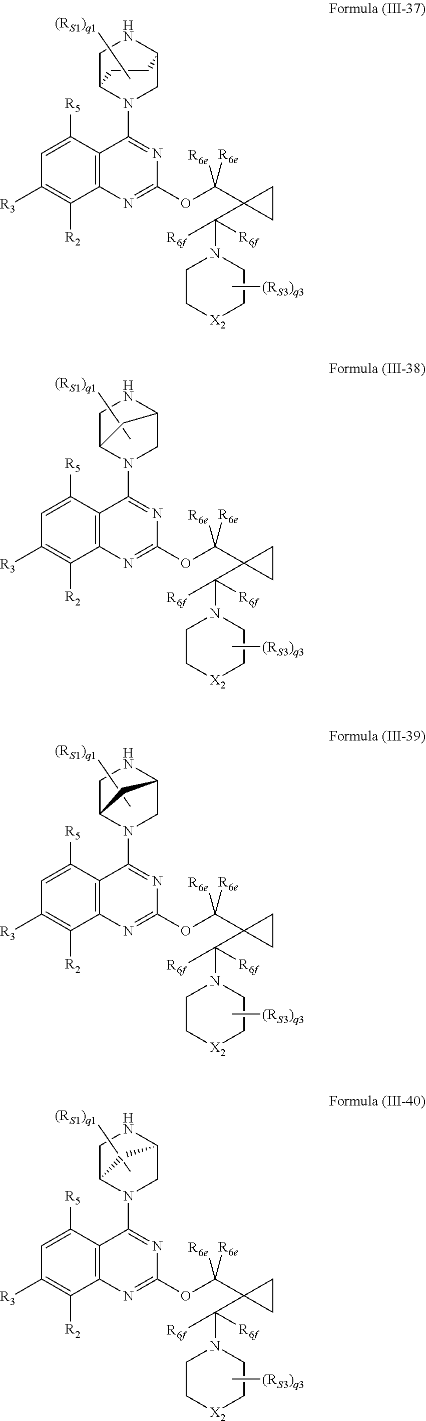

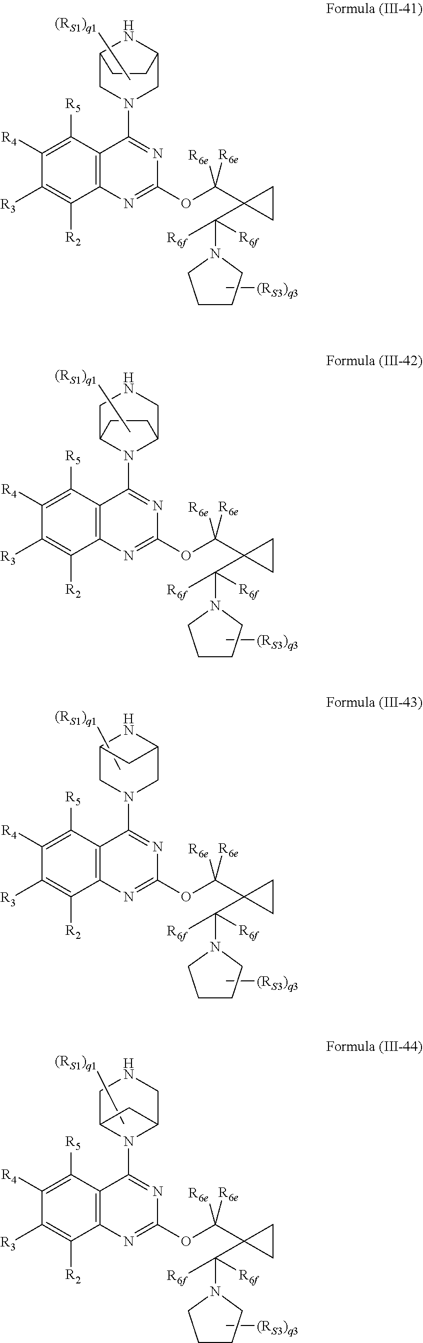

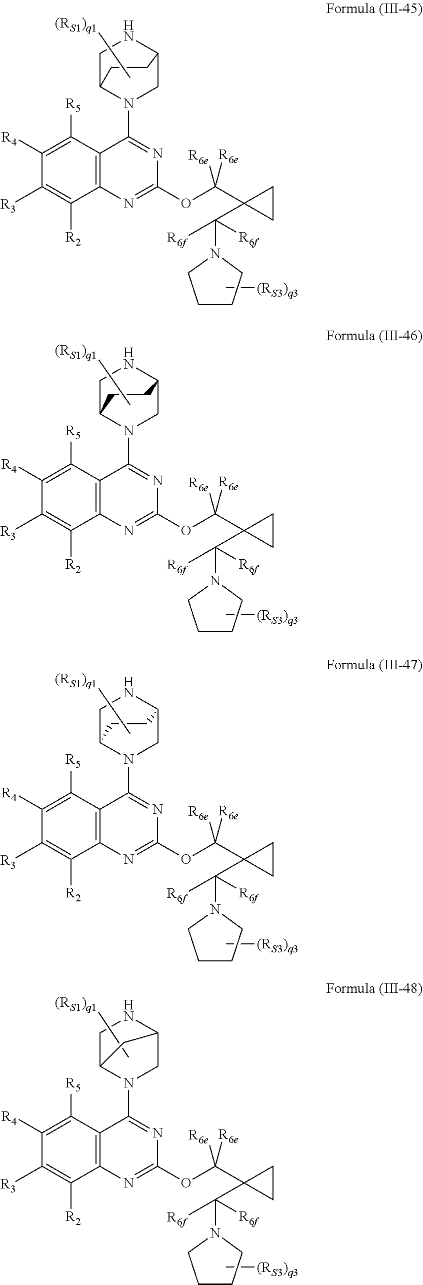

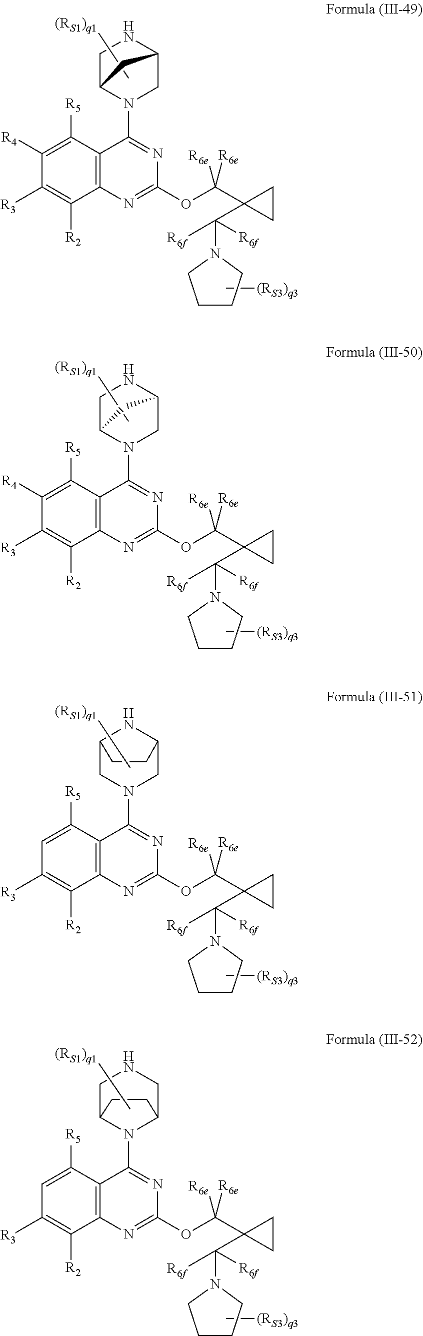

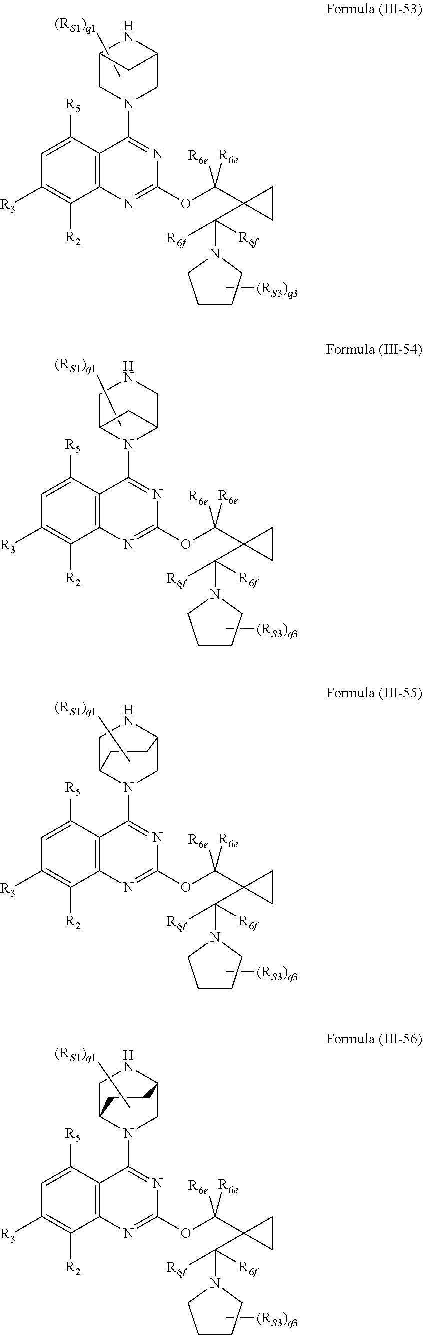

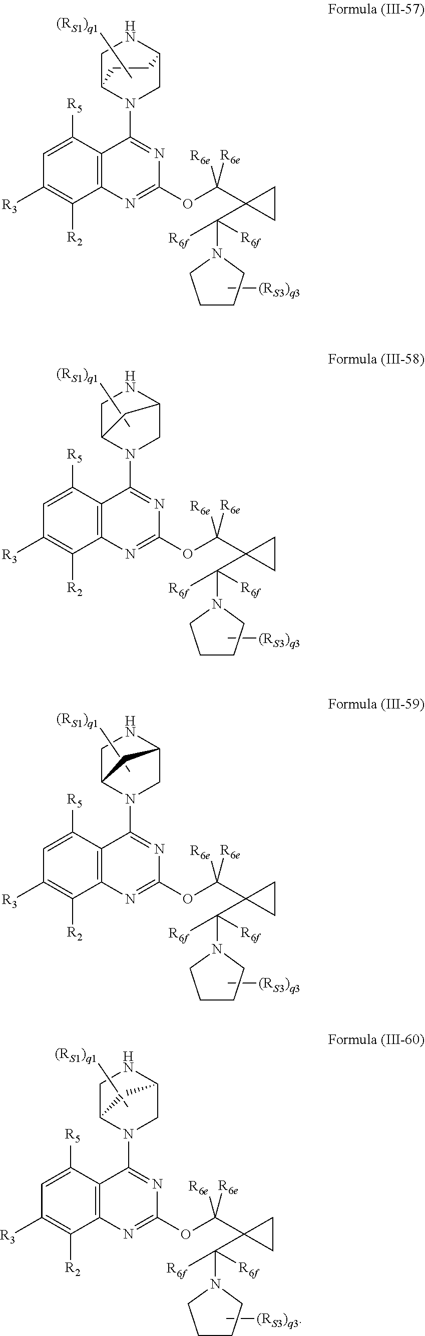

34. The compound of claim 27, wherein,
R₃ is selected from phenyl or naphthyl, wherein said Phenyl or naphthyl is optionally independently substituted with one or more R₃₁;
R₃₁ at each occurrence is independently selected from —OH, —C≡CH, —F, —Cl, —CH₃, —CH₂CH₃, —S—CH₃, —O—CH₃, —CF₃,

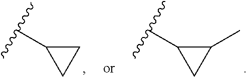

35. The compound of claim 27, wherein, R₃ is selected from

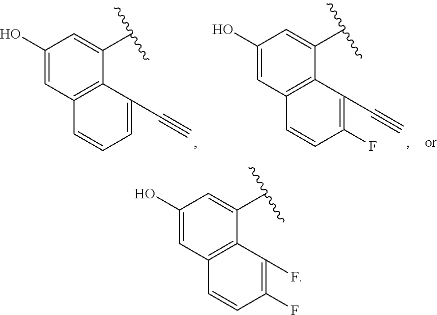 [SMILES: *c1cc(O)cc2cccc(C#C)c12 ; *c1cc(O)cc2ccc(F)c(C#C)c12 ; *c1cc(O)cc2ccc(F)c(F)c12]

36. The compound of claim 27, wherein,
the compound is selected from any one of formula (IV-1) to (IV-180):

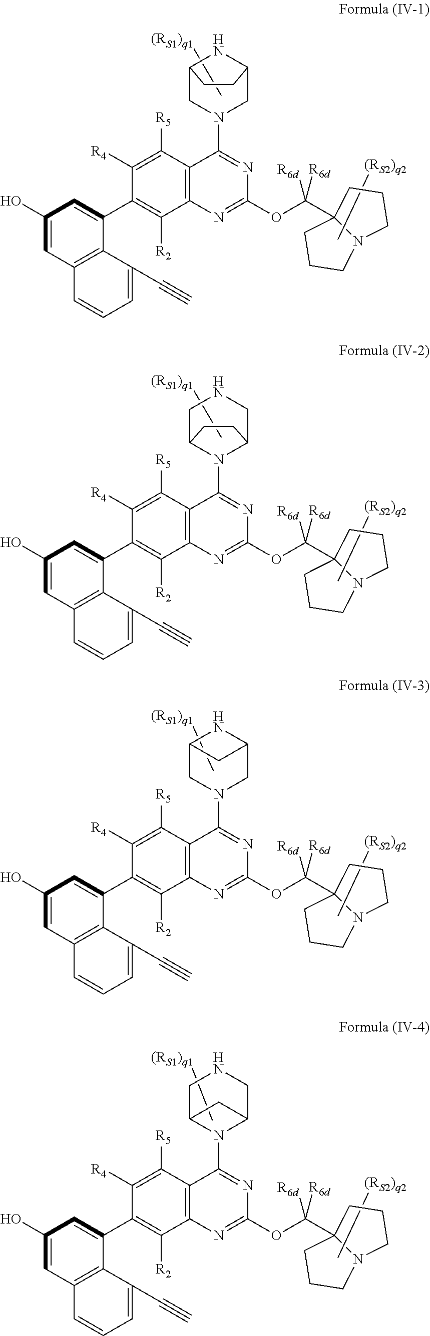

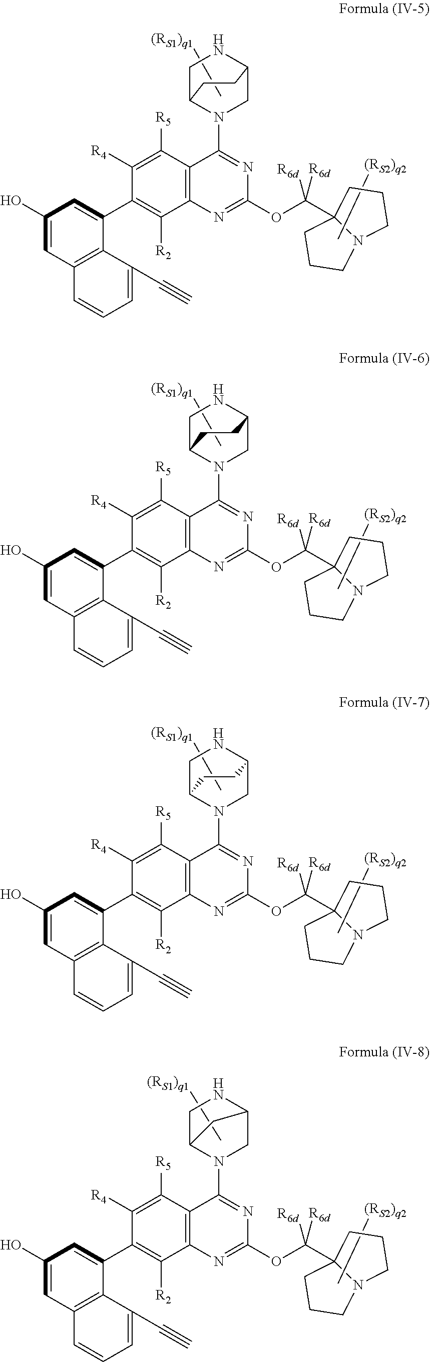

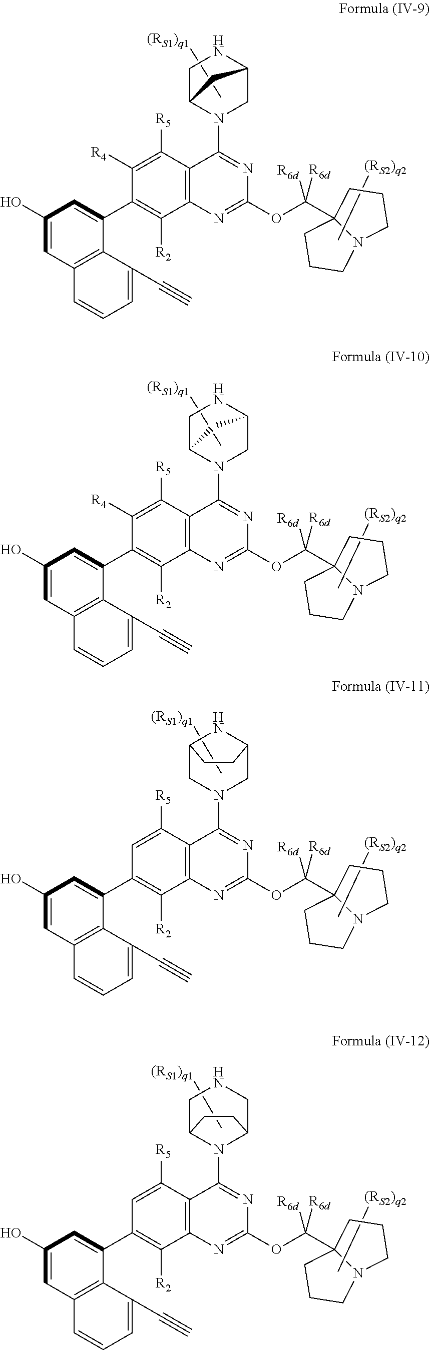

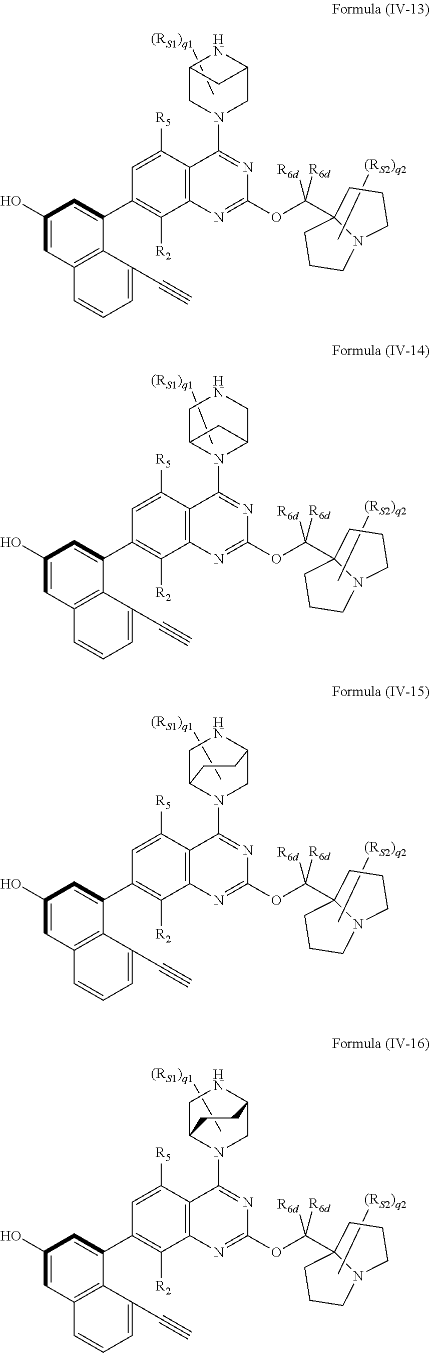

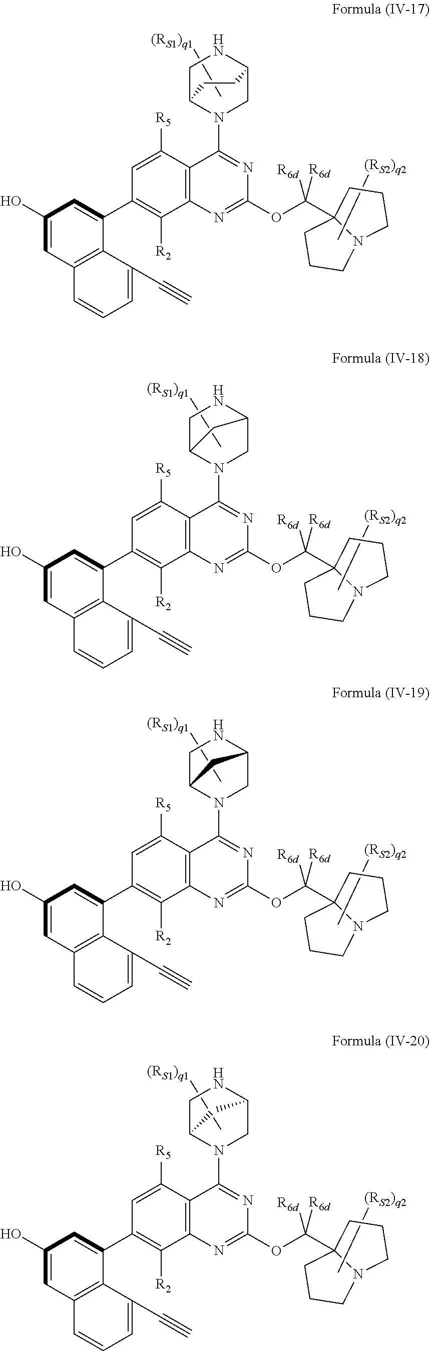

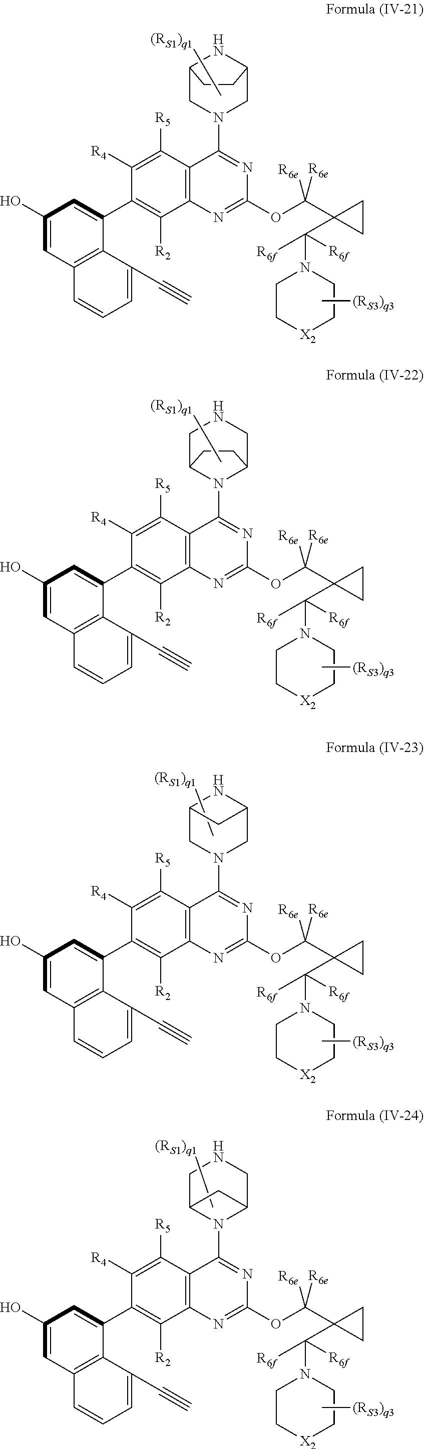

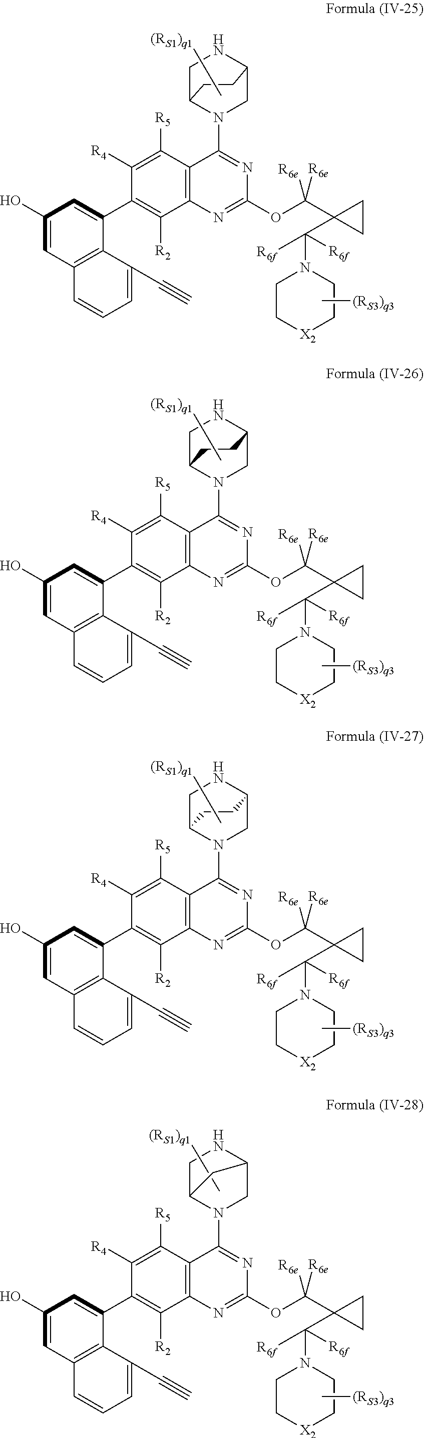

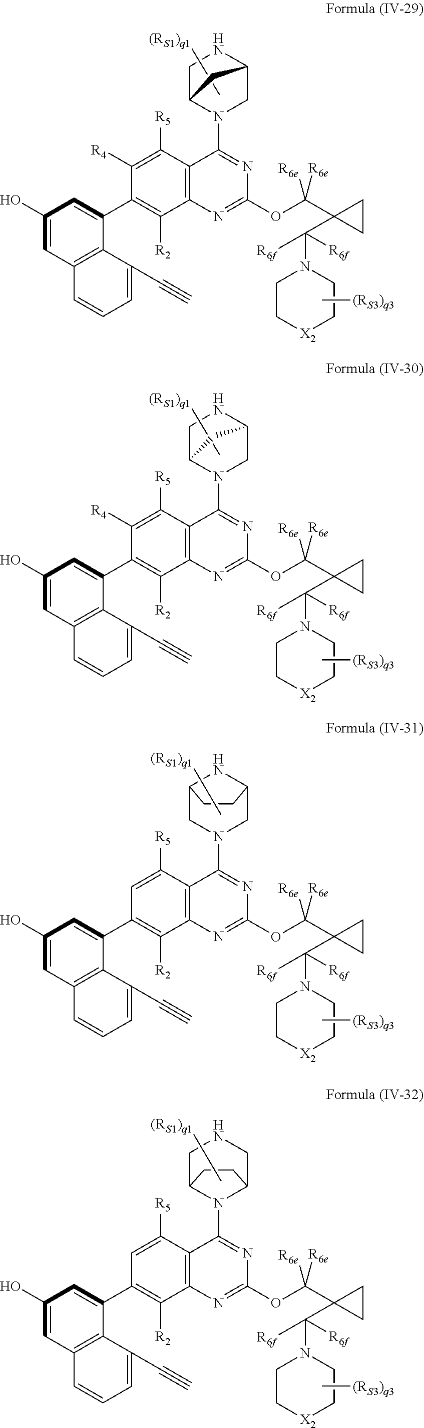

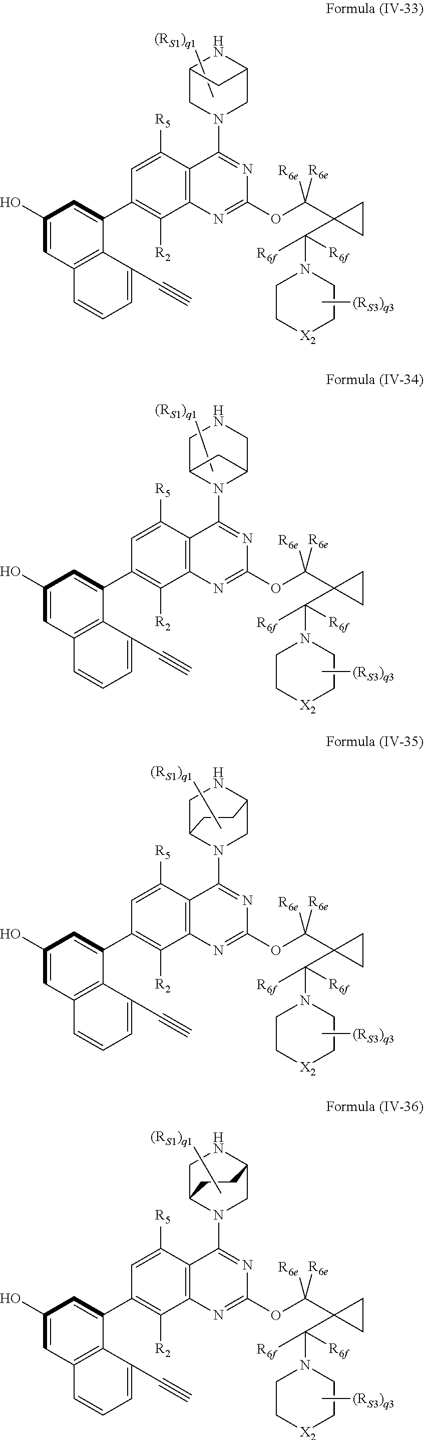

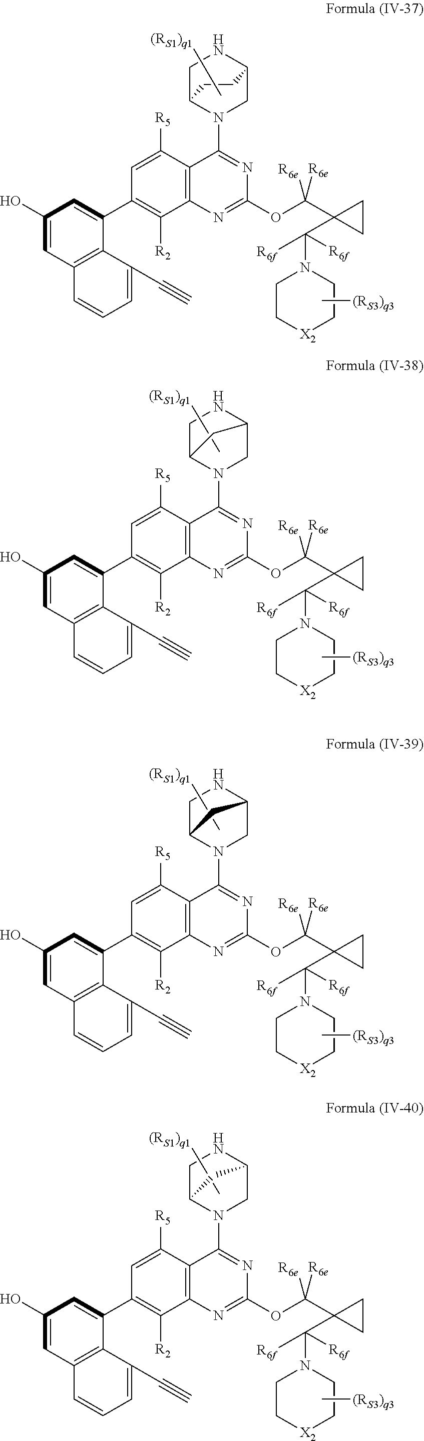

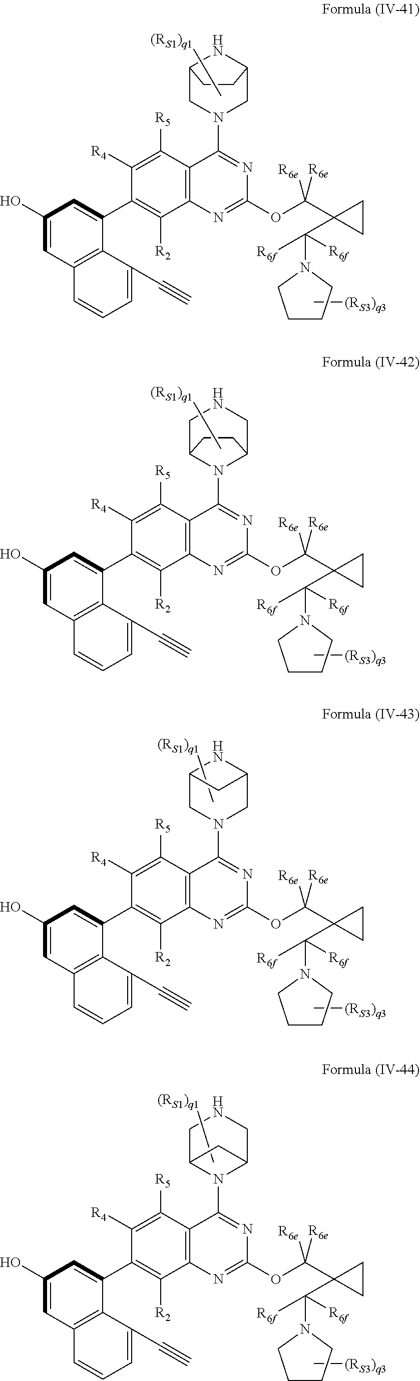

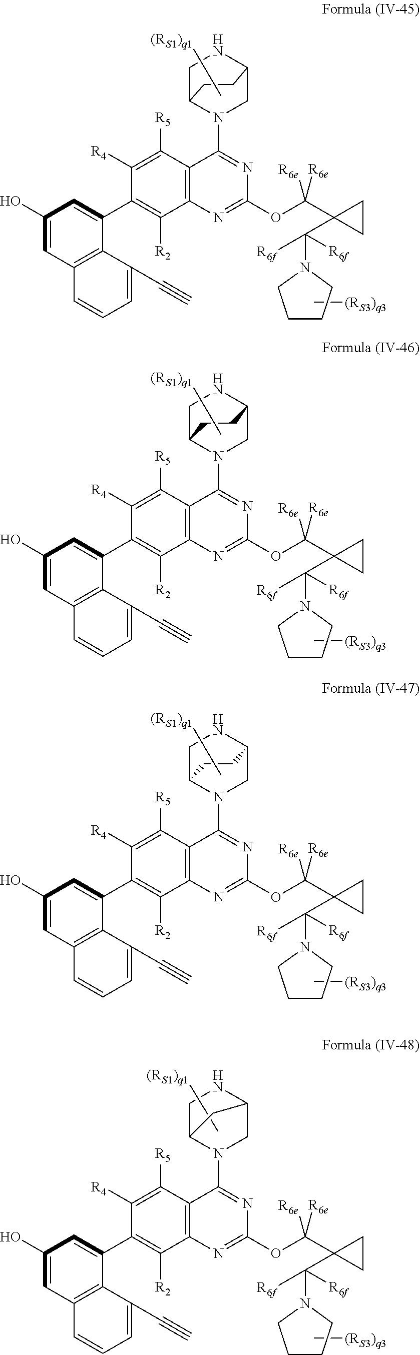

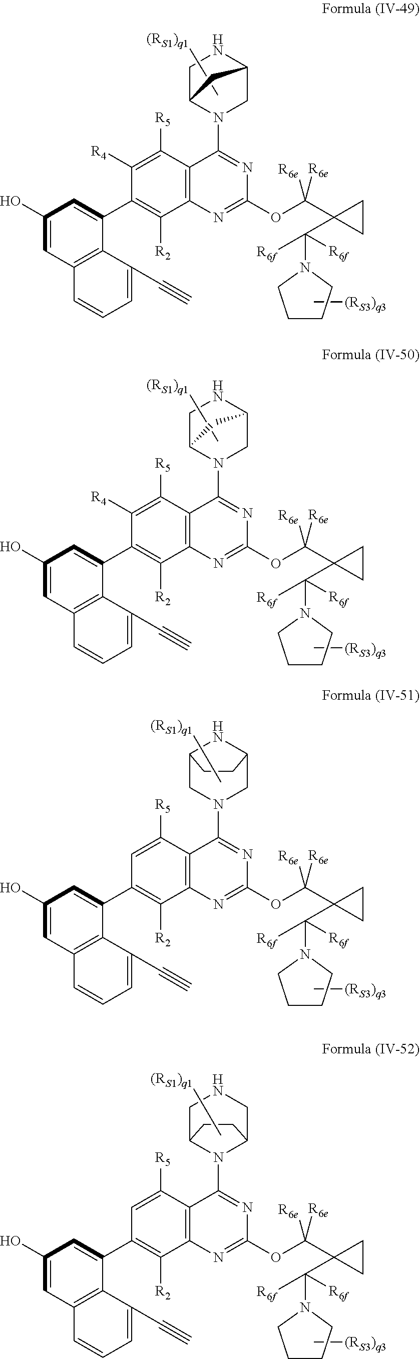

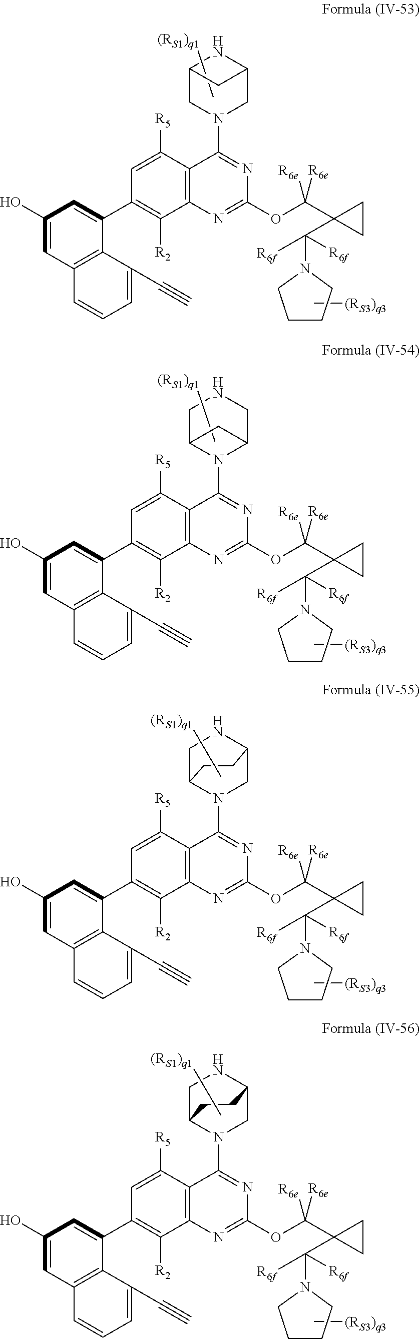

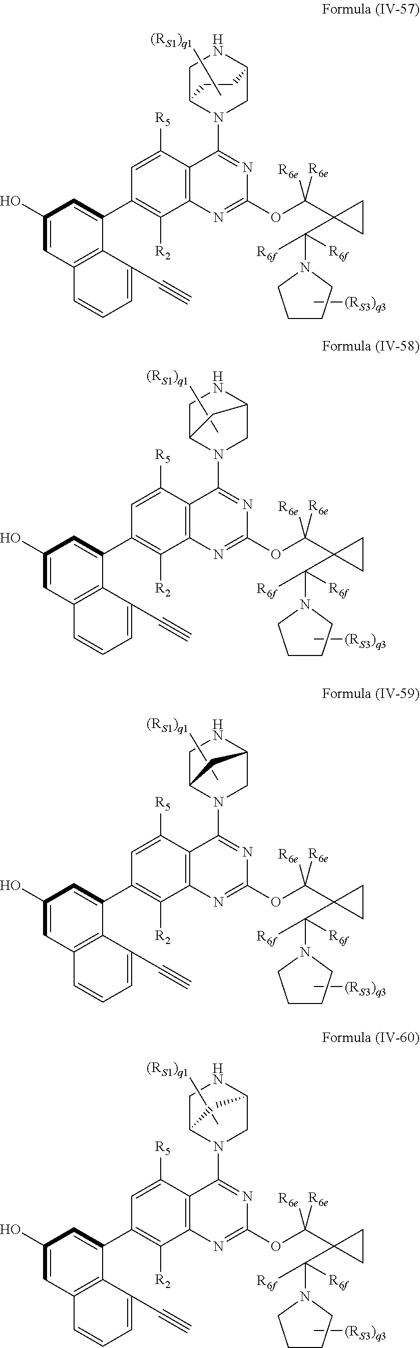

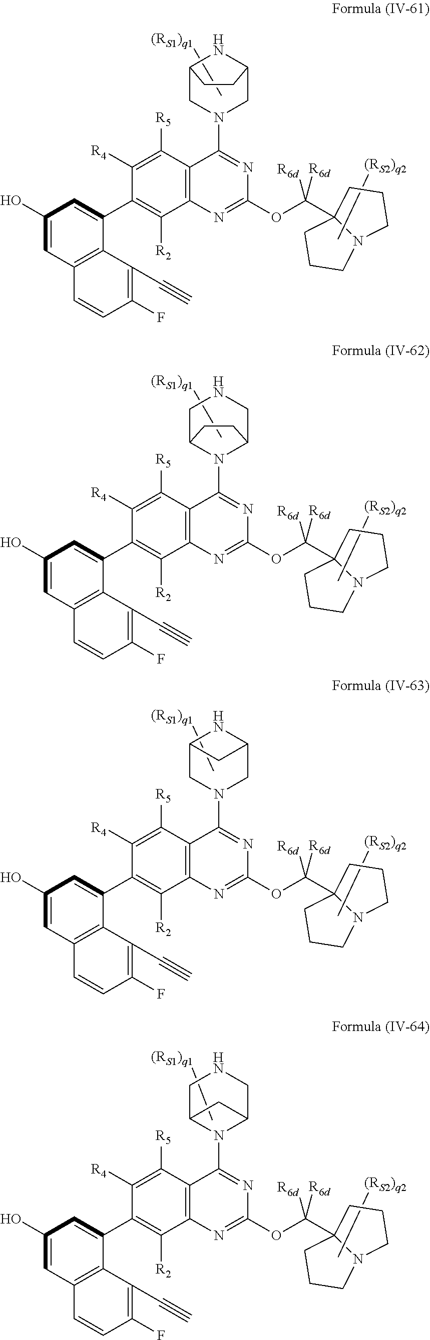

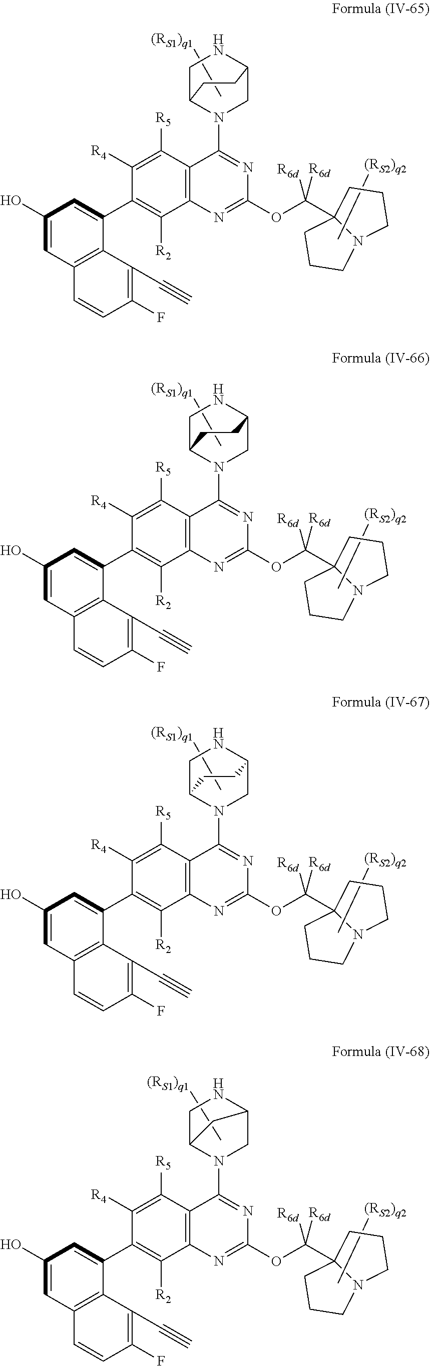

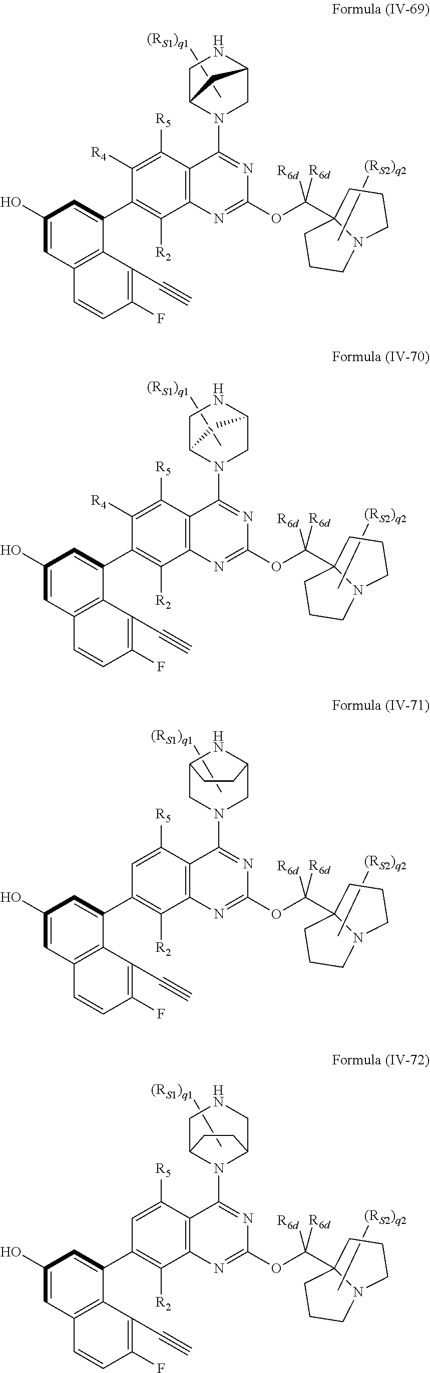

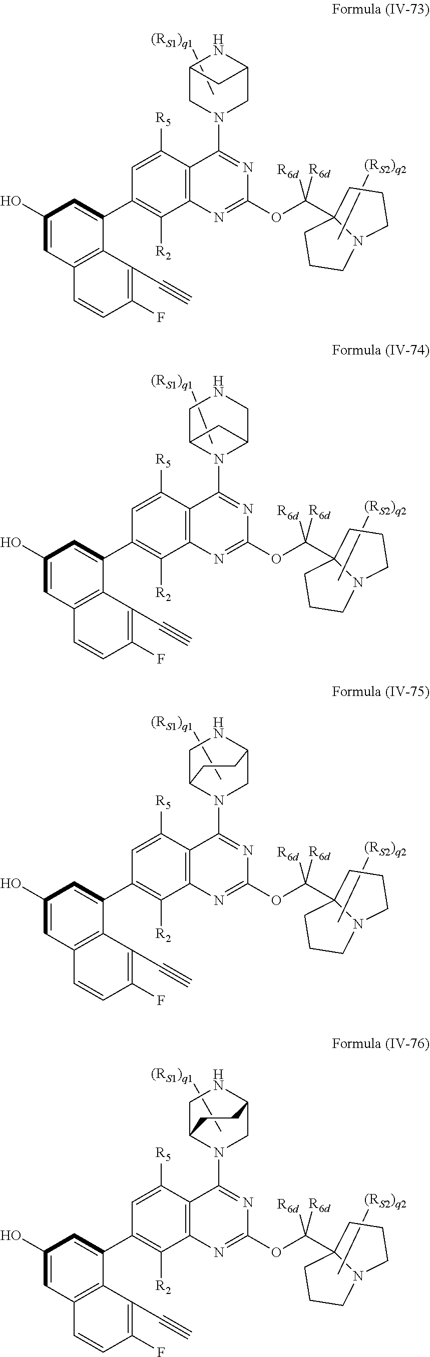

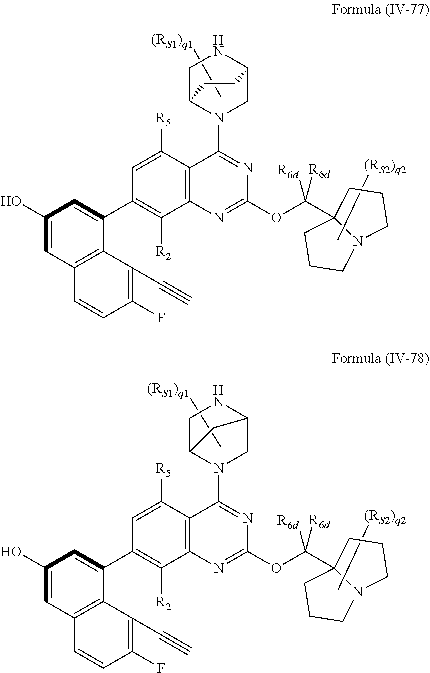

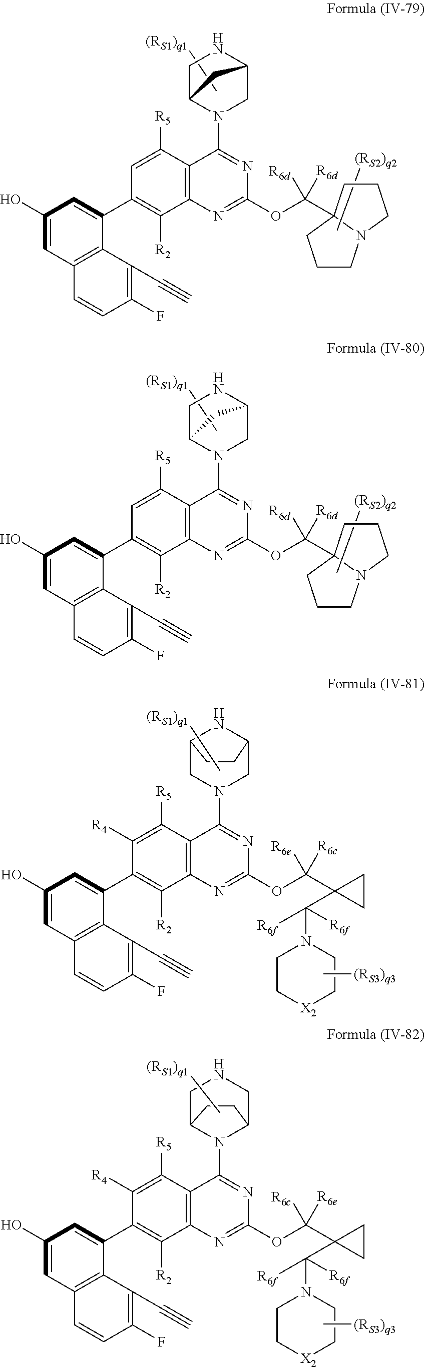

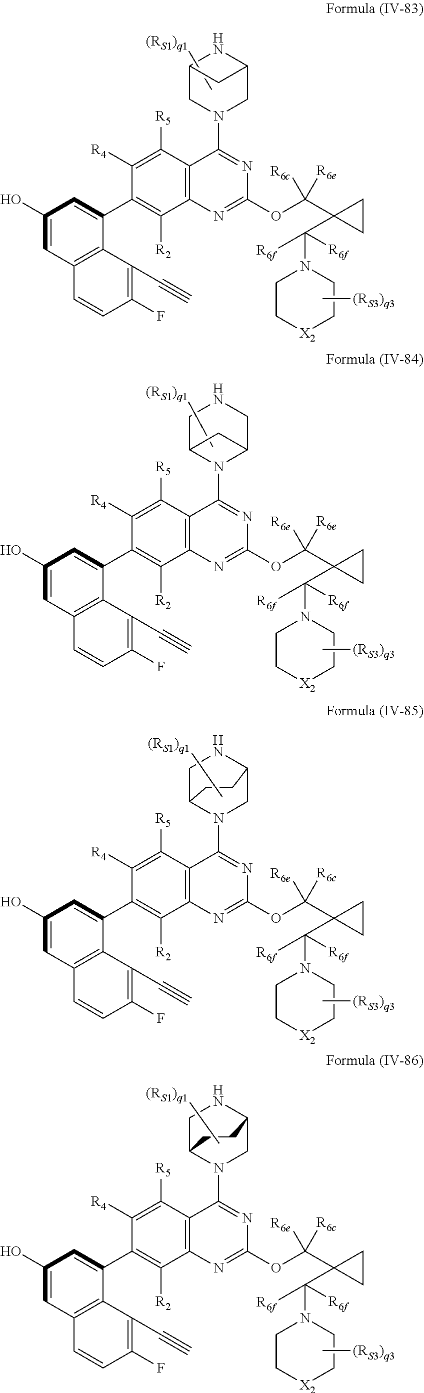

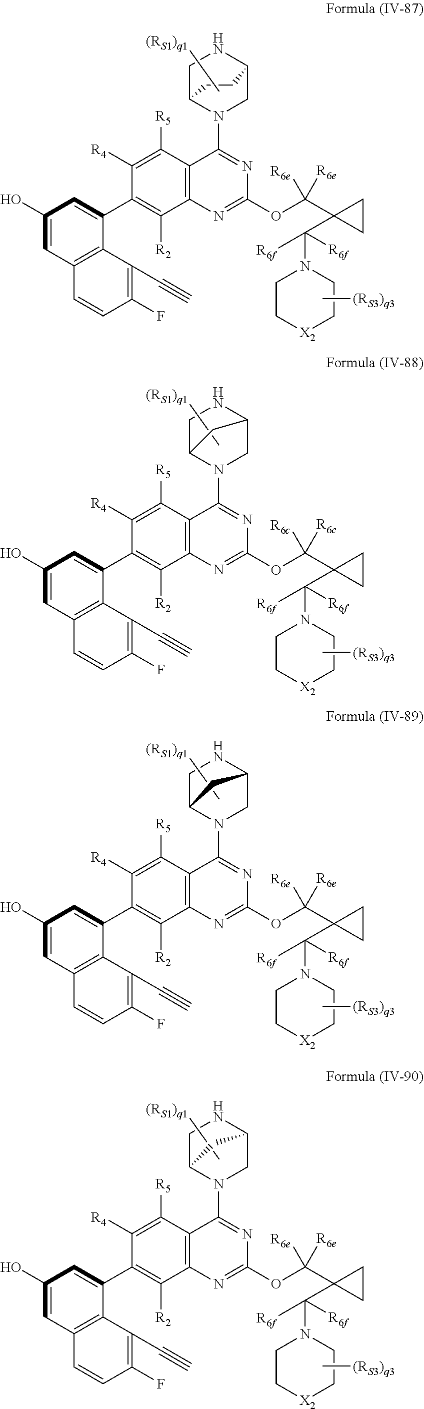

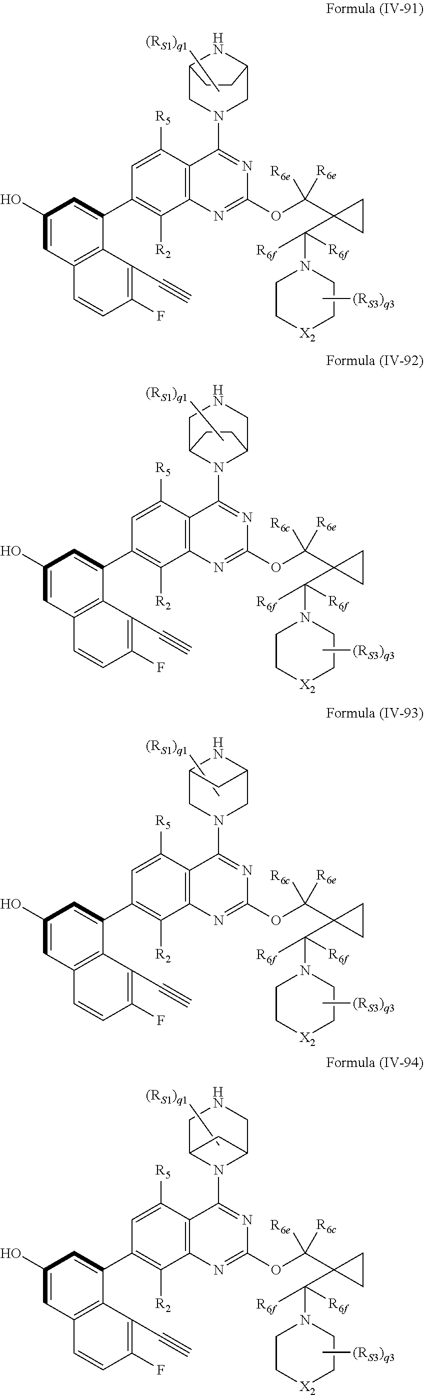

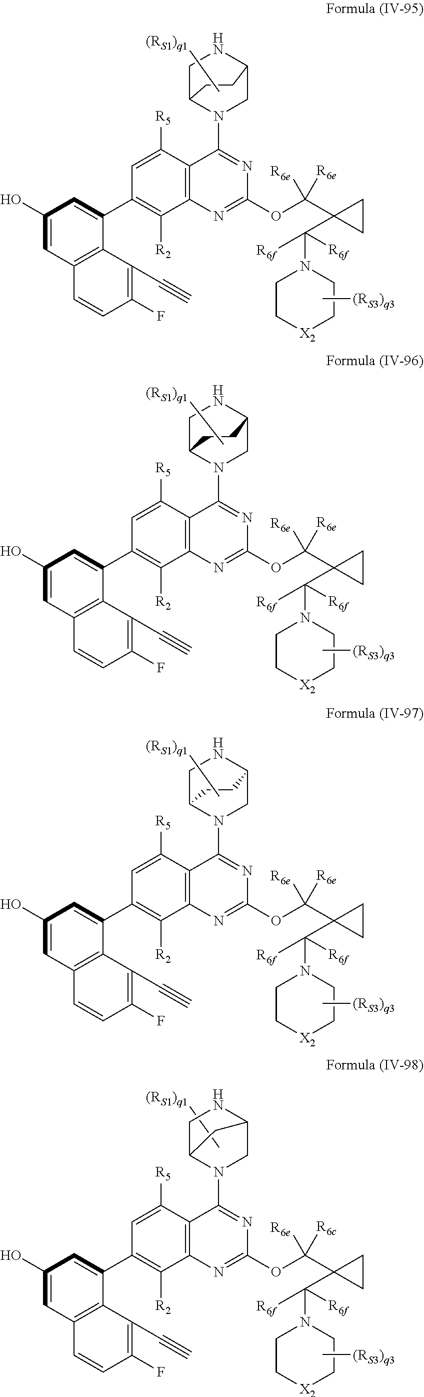

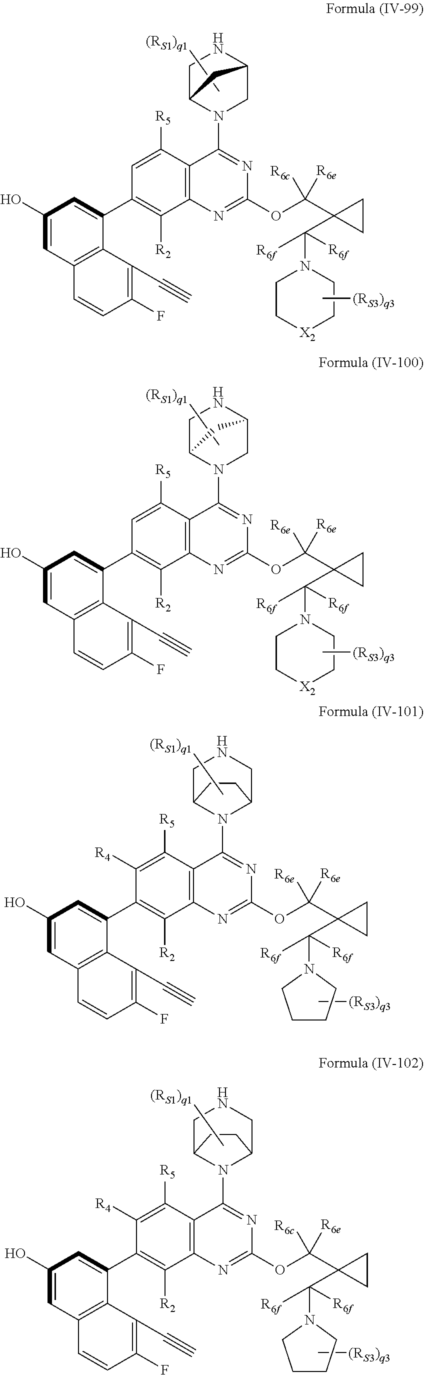

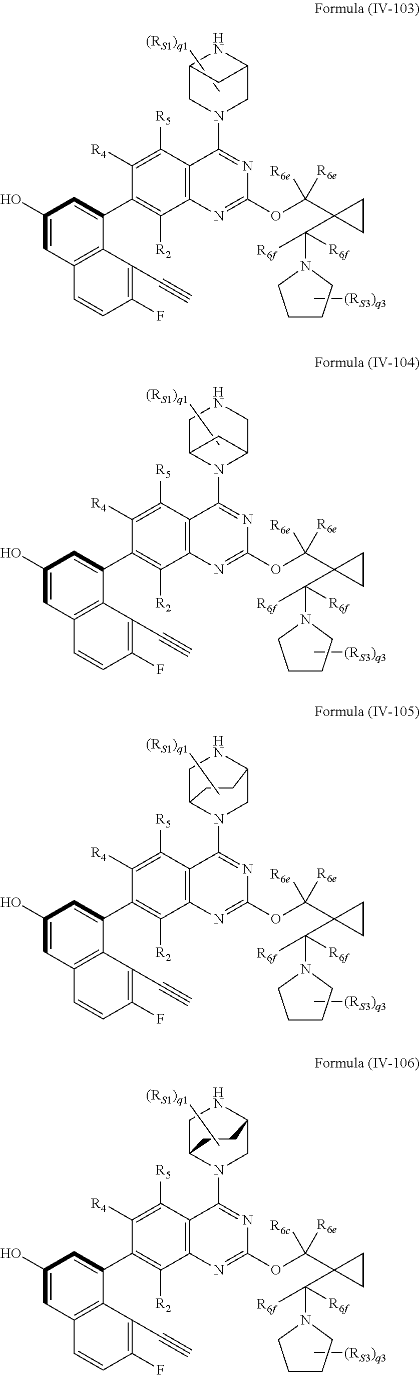

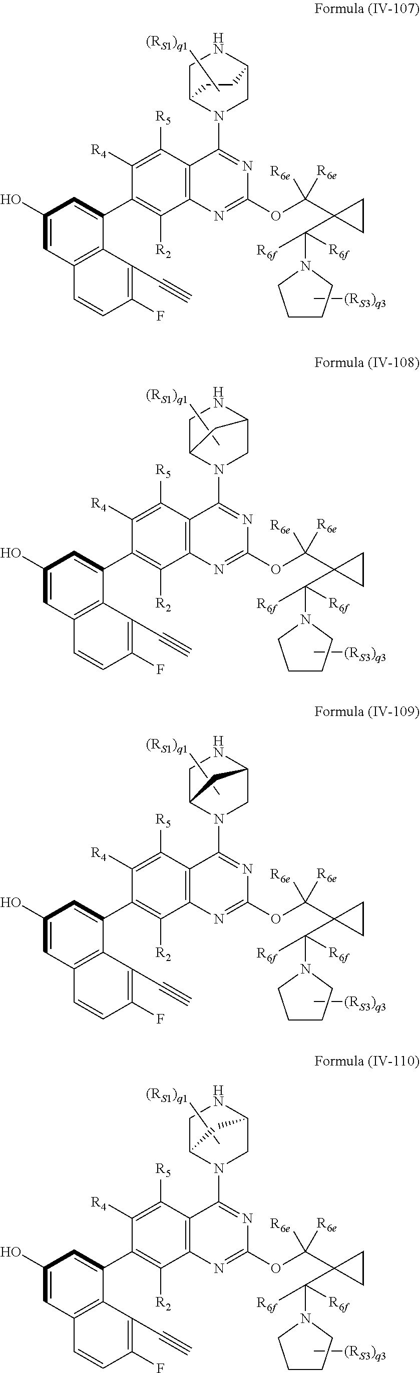

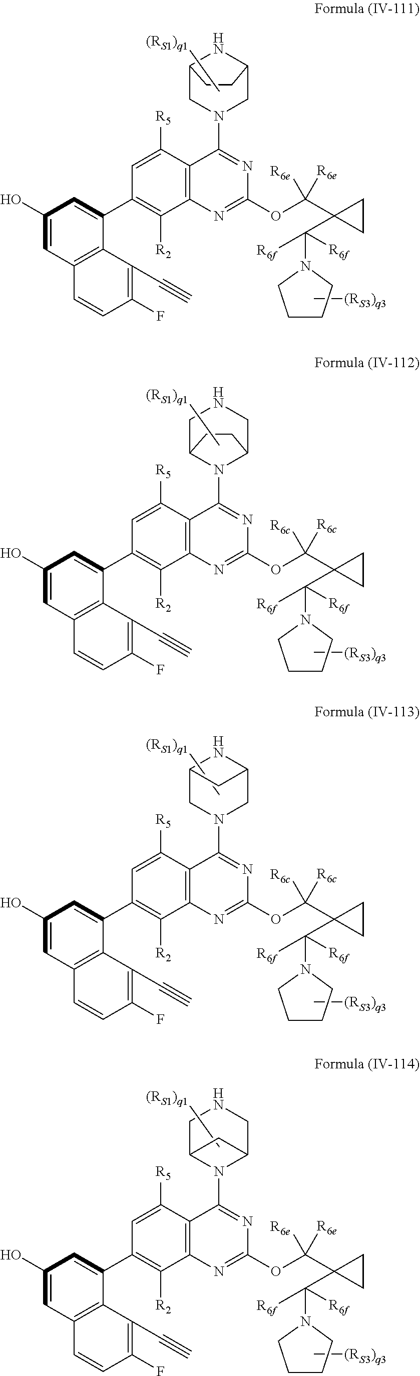

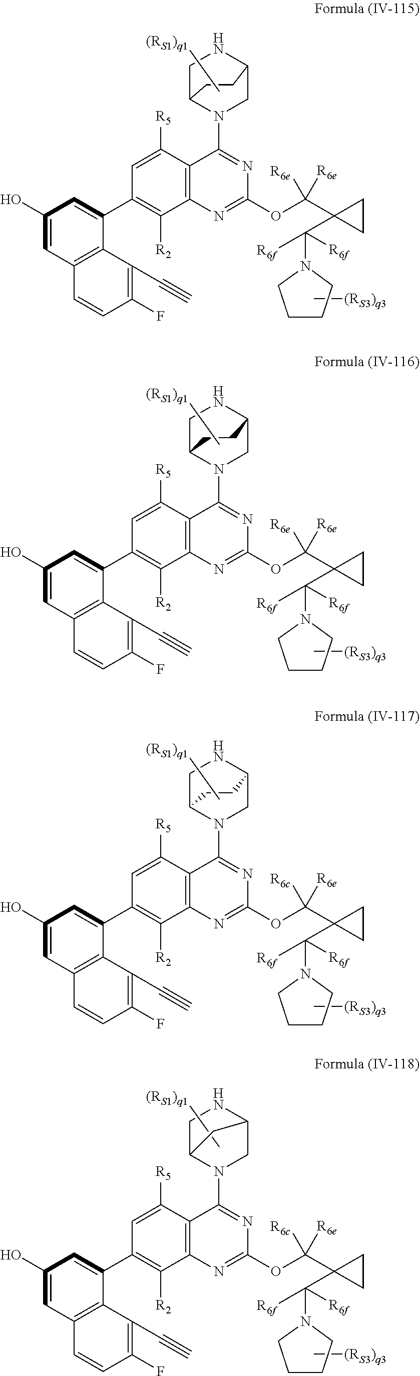

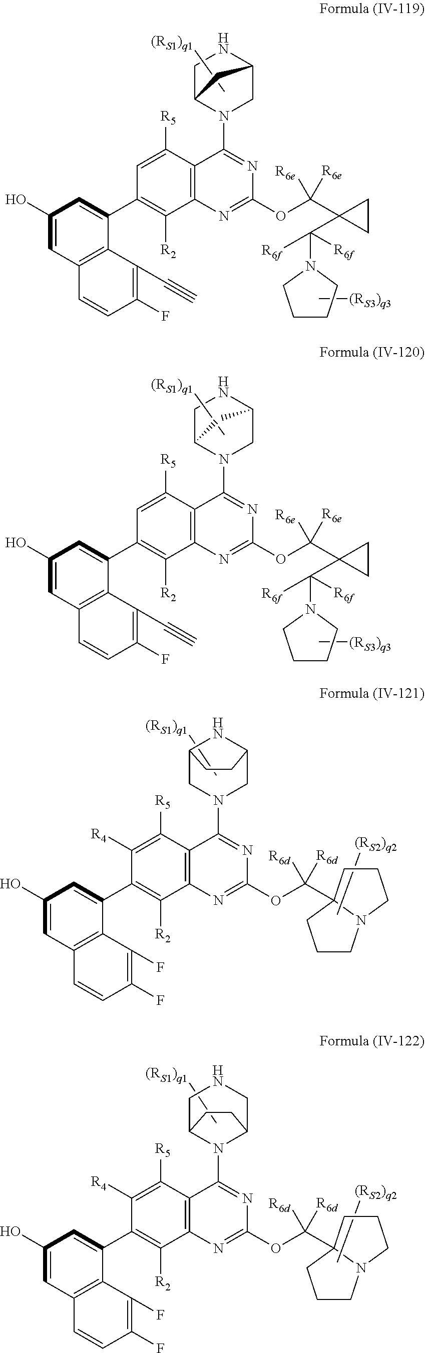

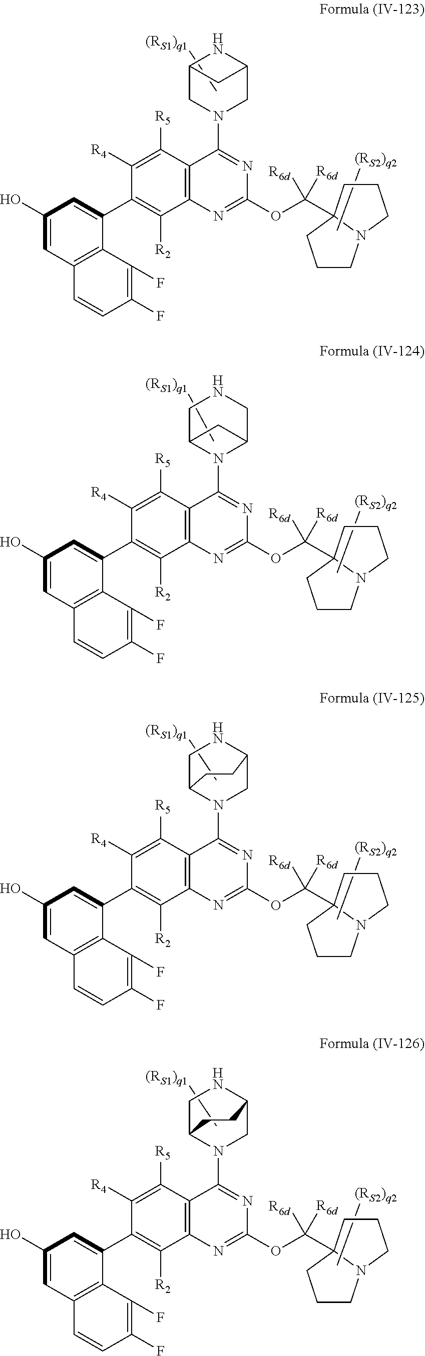

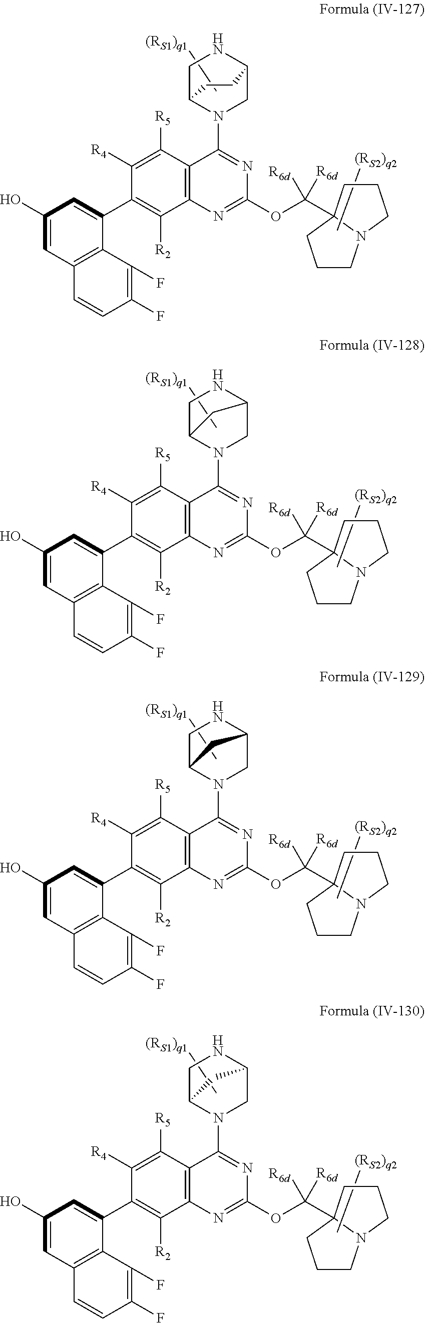

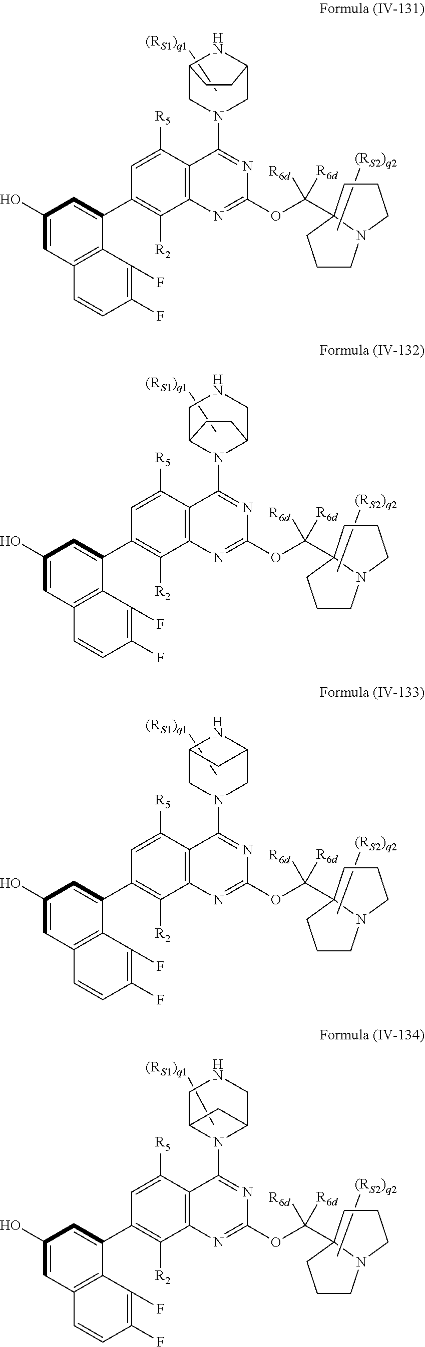

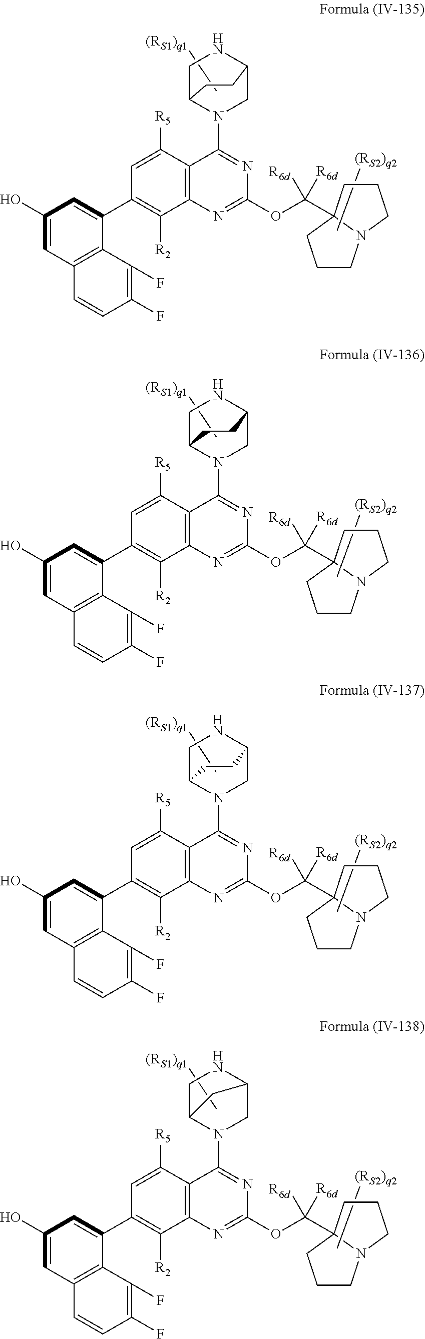

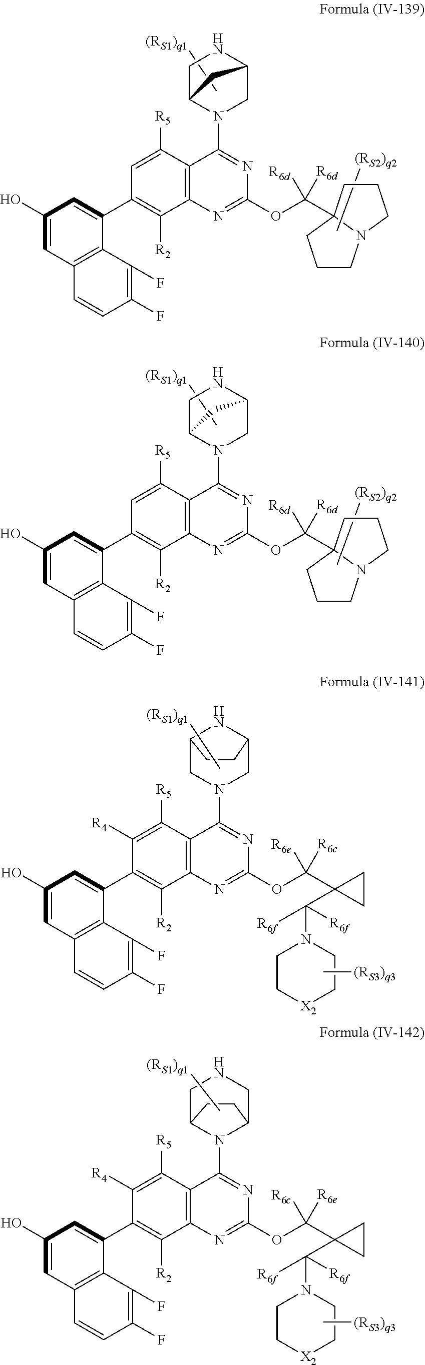

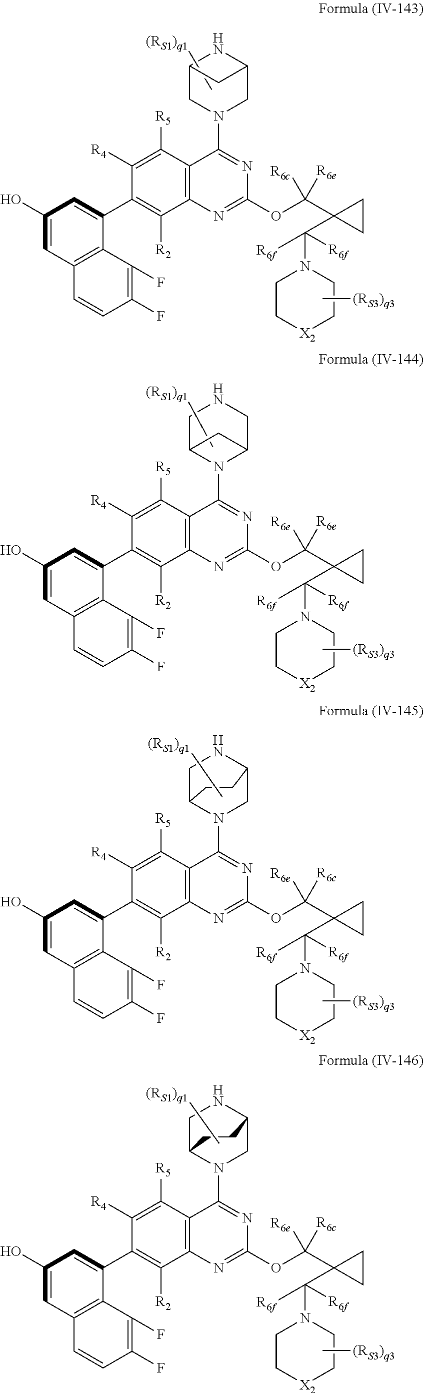

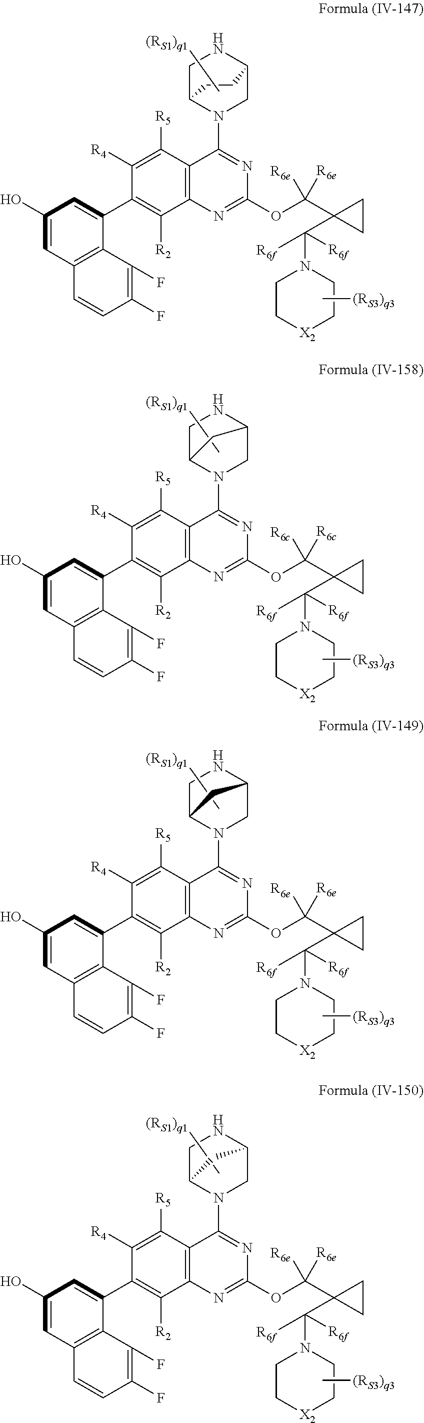

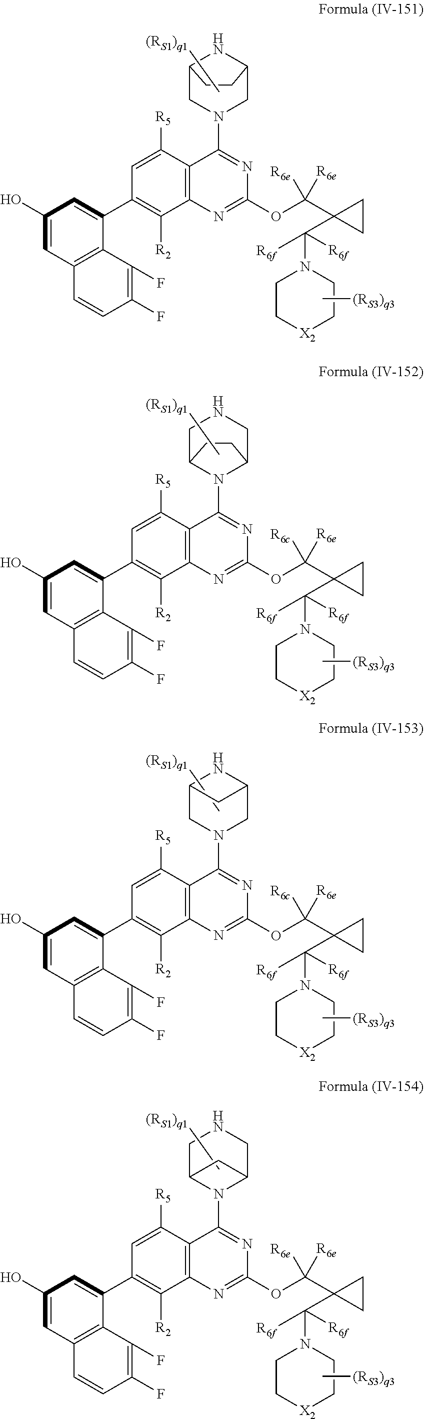

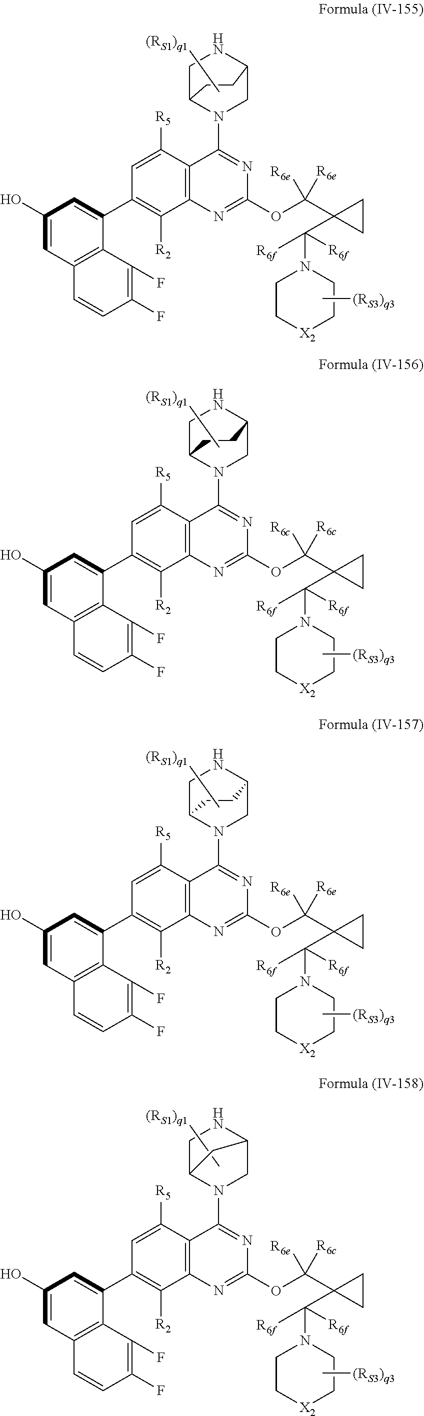

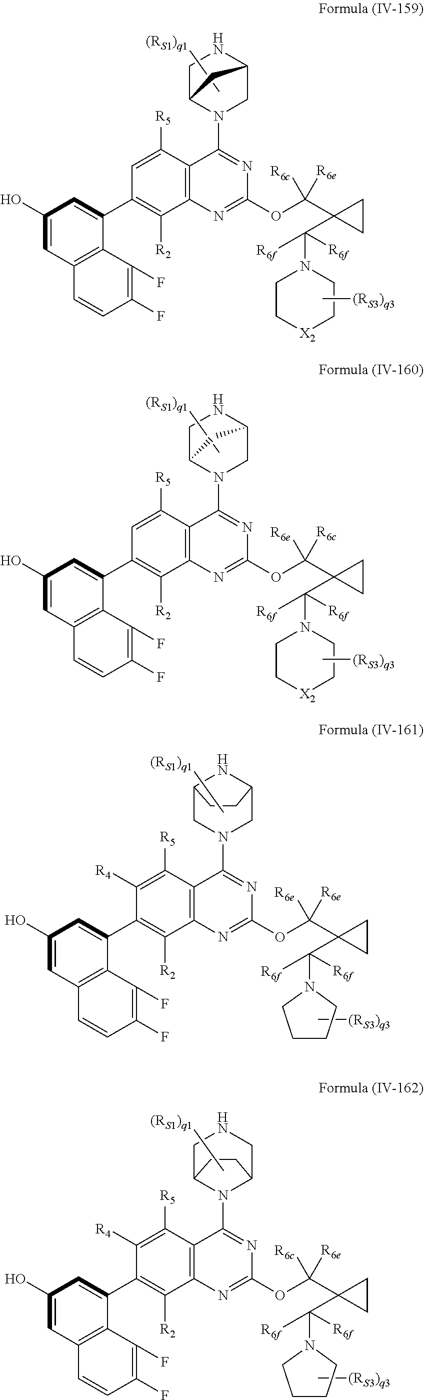

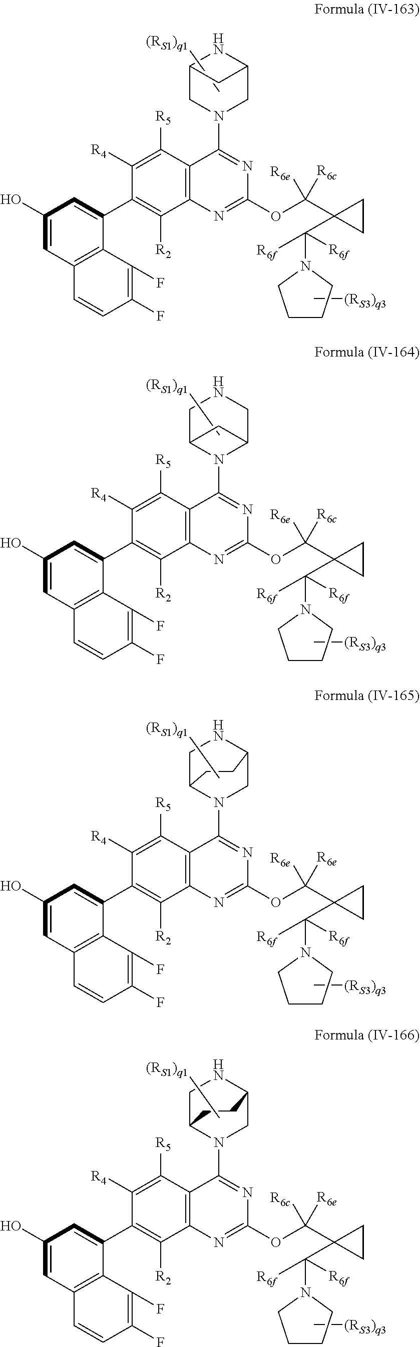

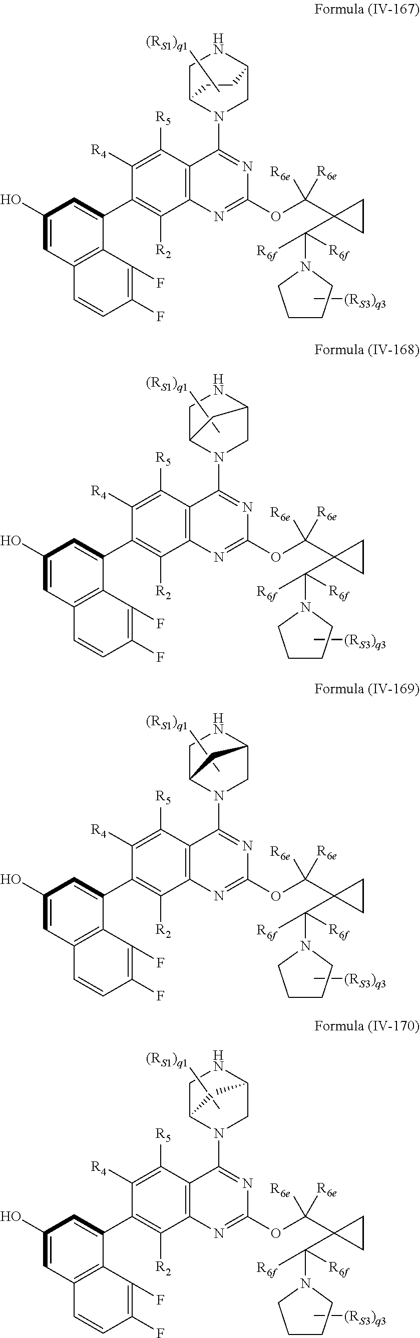

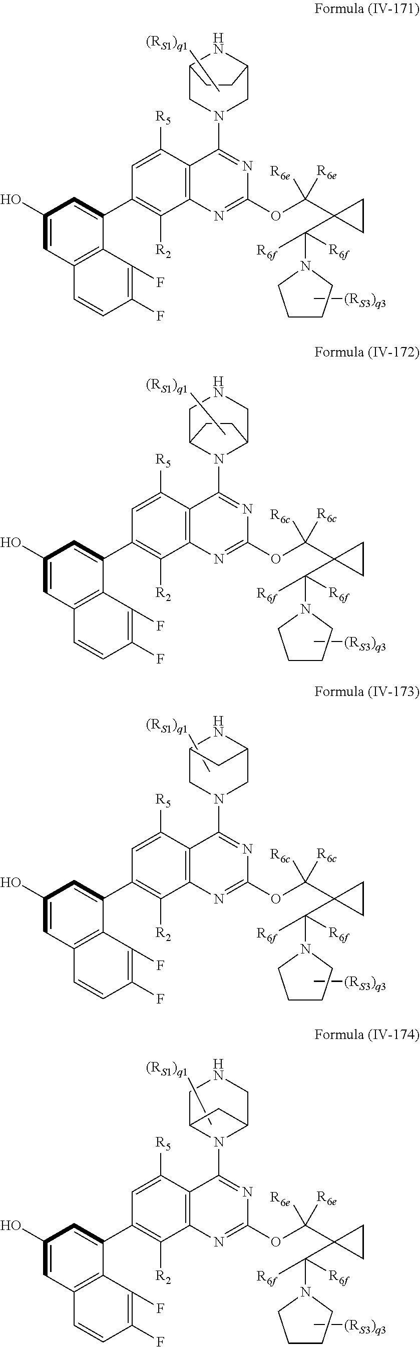

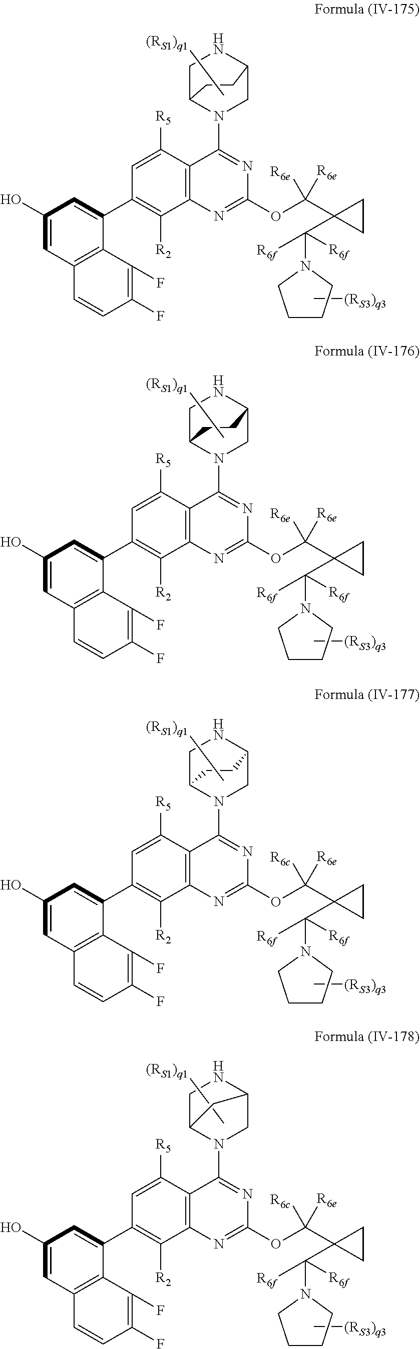

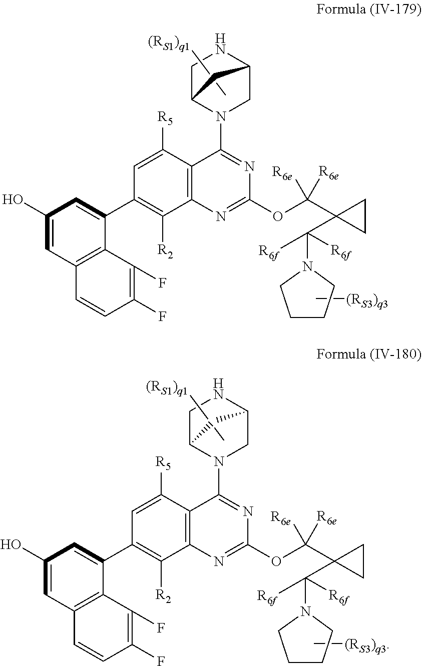

37. The compound of claim 27, wherein,
Wherein the moiety of 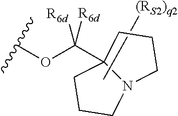
is selected from

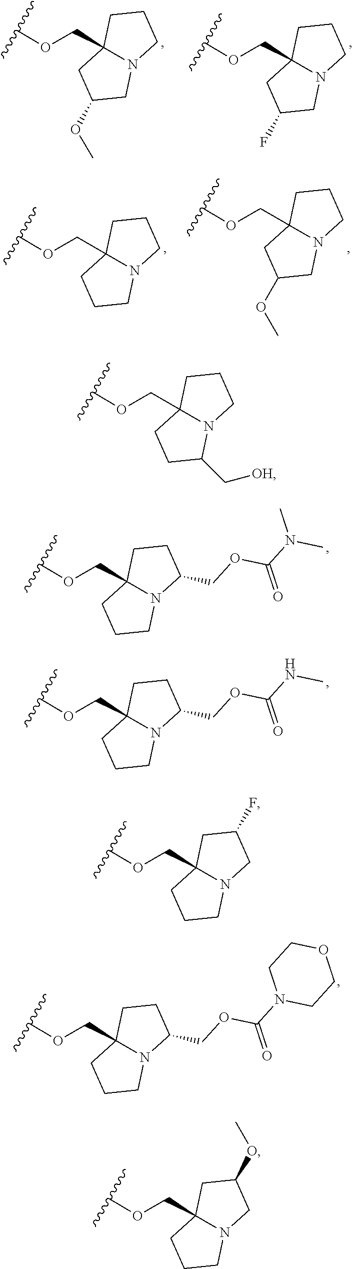

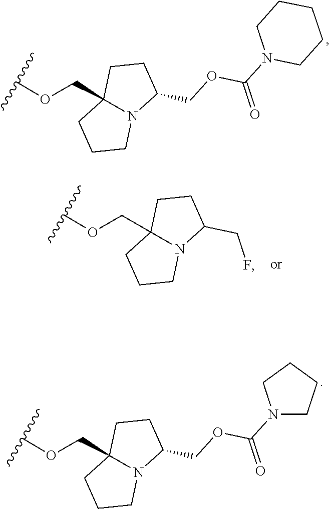 [SMILES: *OC[C@]12CCCN1[C@@H](COC(=O)N1CCCCC1)CC2 ; *OCC12CCCN1C(CF)CC2 ; "or" ; *OC[C@]12CCCN1[C@@H](COC(=O)N1CCCC1)CC2]

38. The compound of claim 27, wherein,
the moiety of 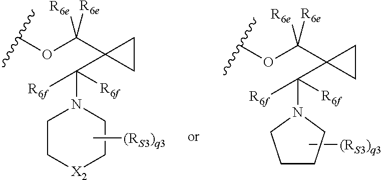
is selected from

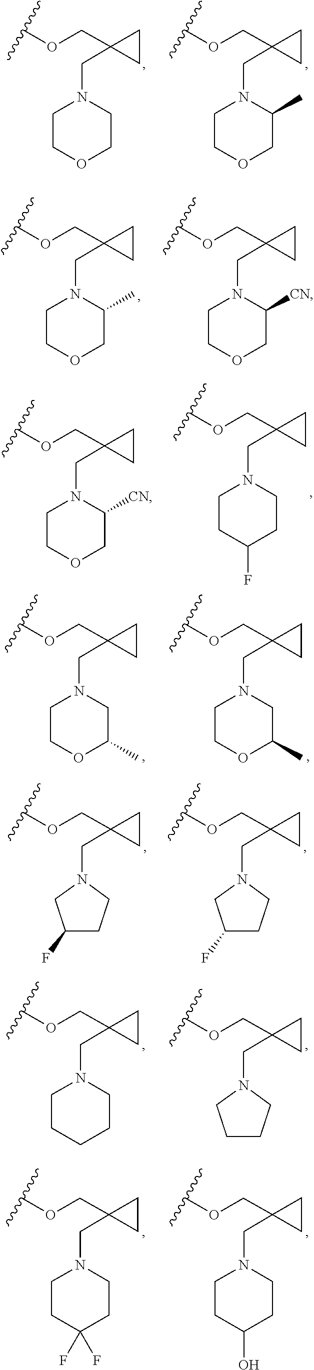

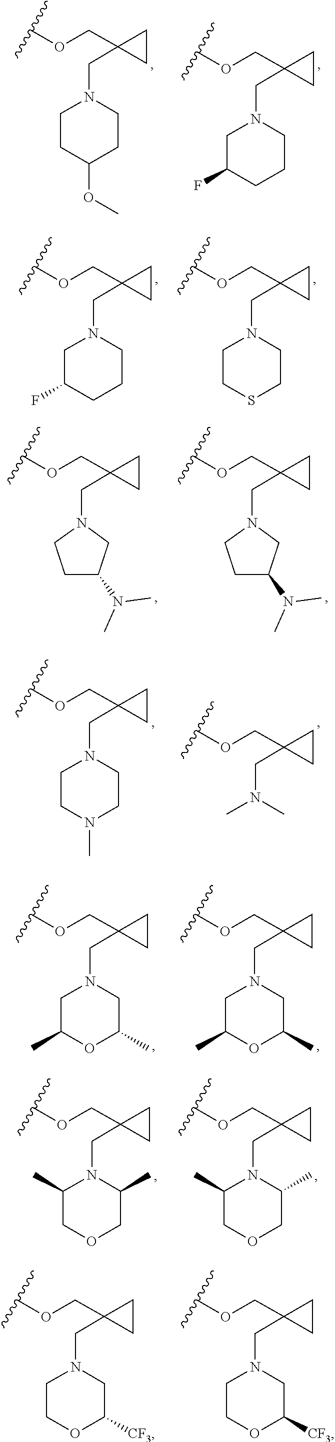

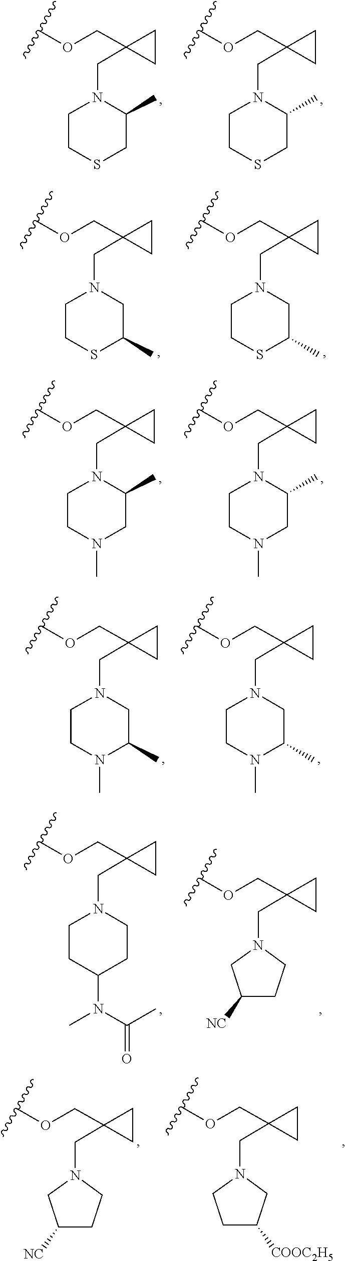

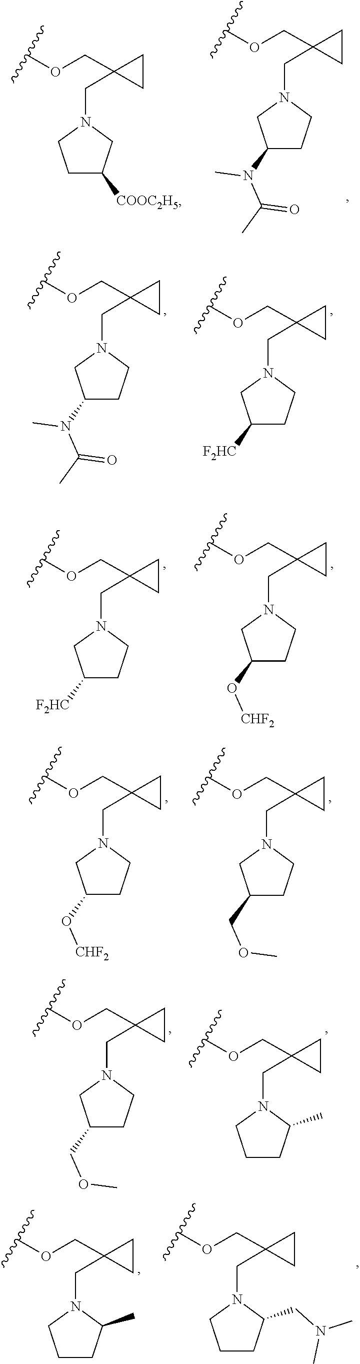

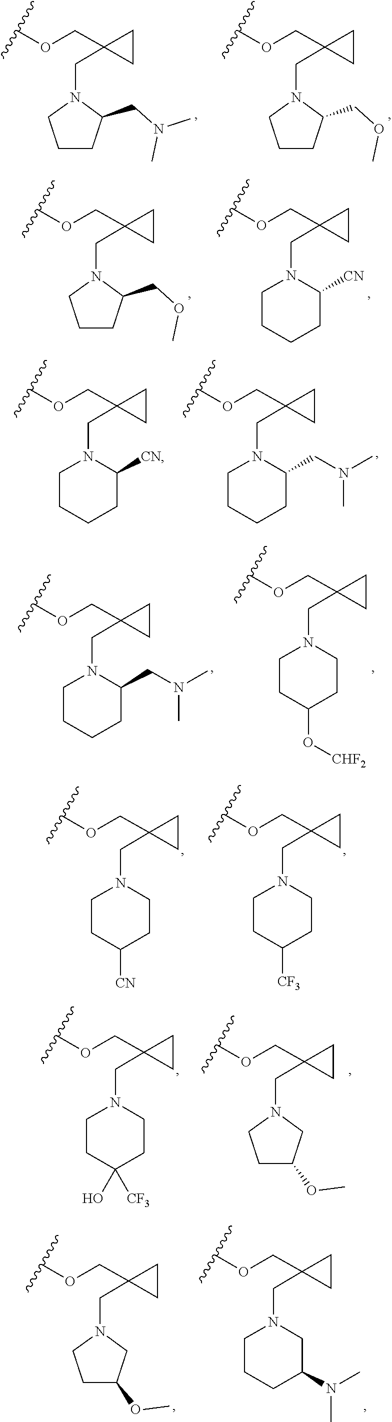

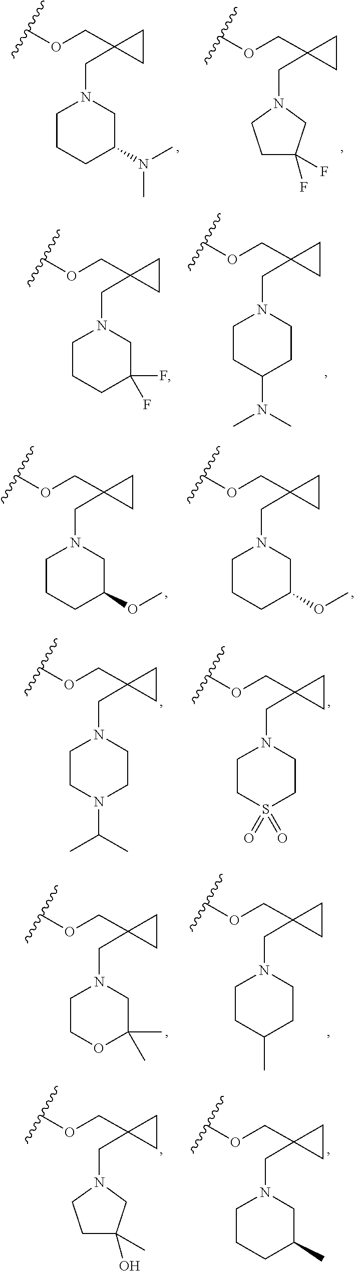

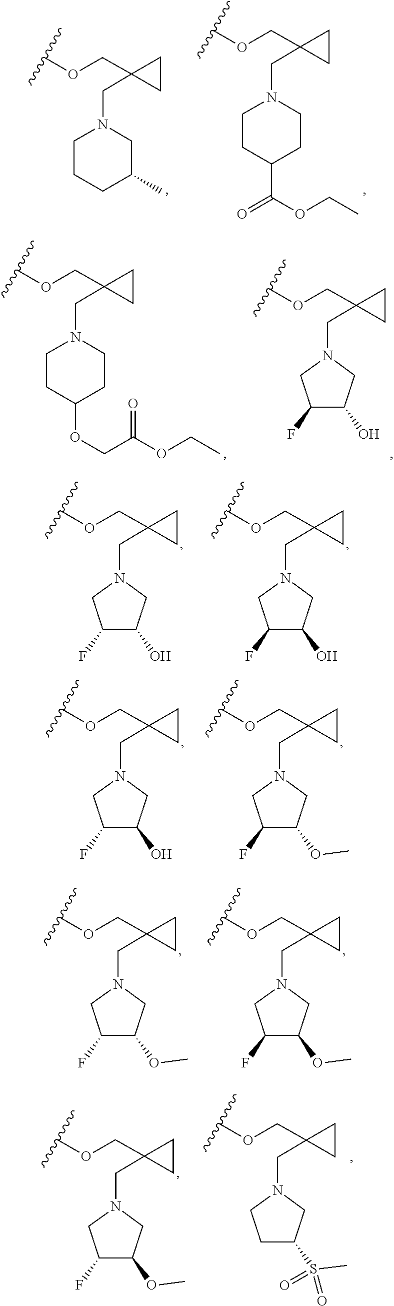

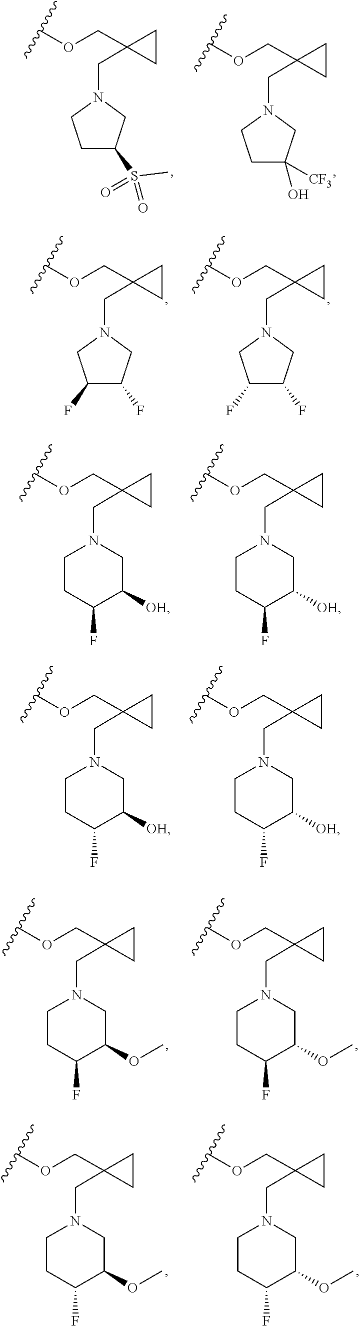

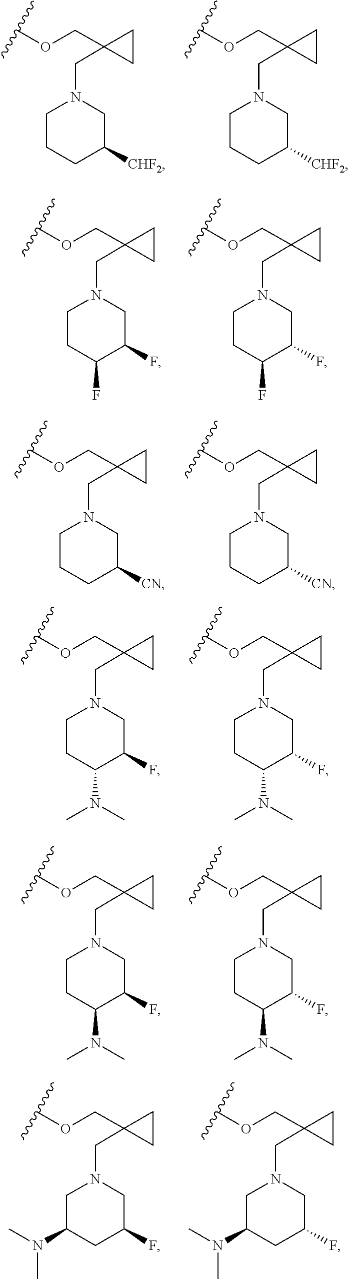

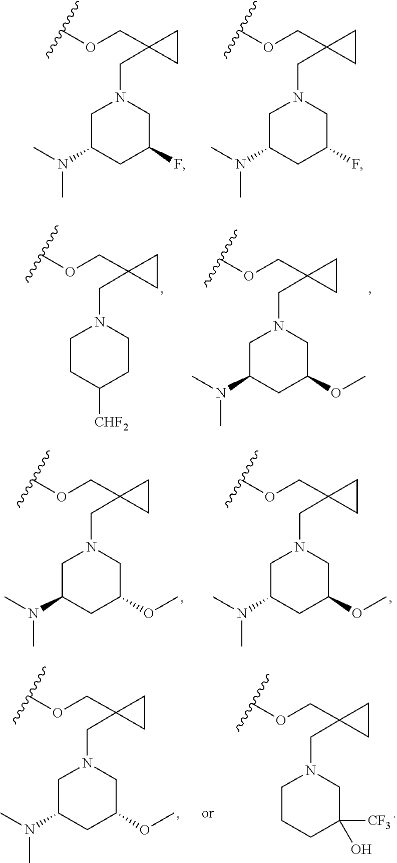

39. The compound of claim 27, wherein,
R₂ is selected from 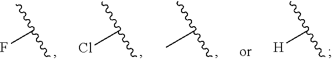
R₅ is hydrogen;
q₁ is 0.

40. The compound of claim 27, wherein,
the compound is of formula (V-1): 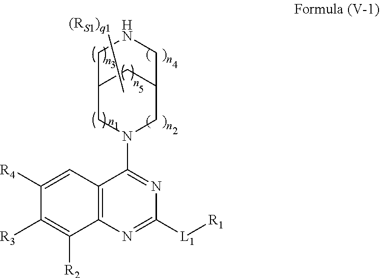
Wherein,
moiety of 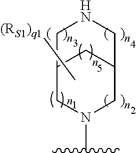
is selected from 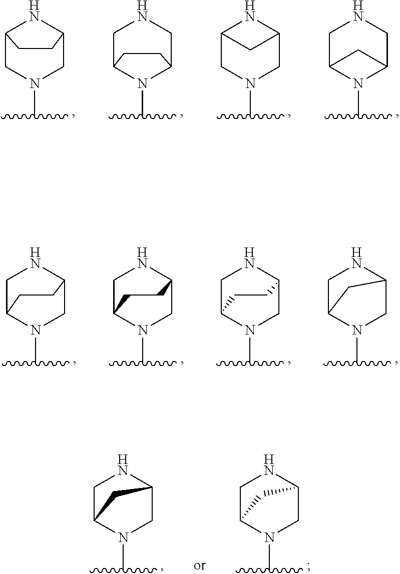
-L₁-R₁ is selected from 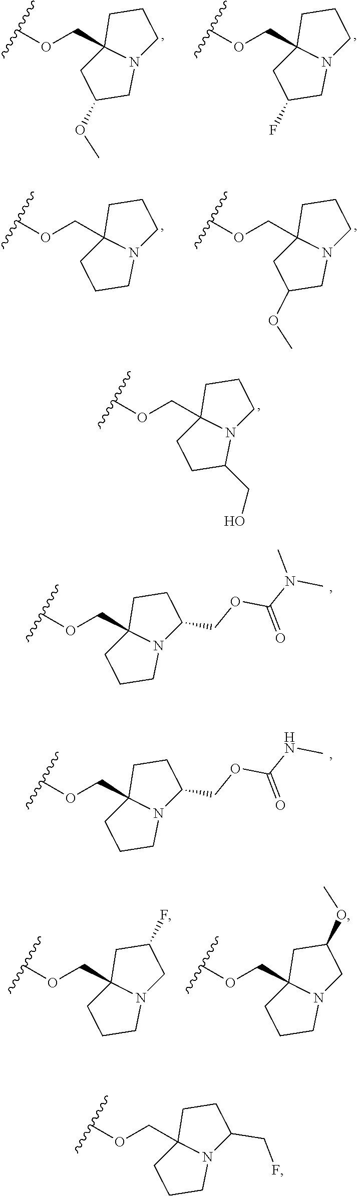 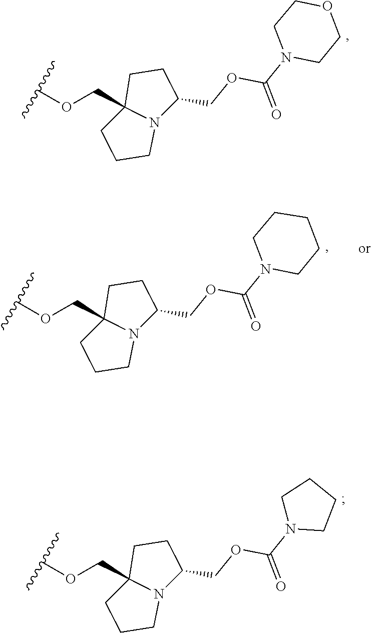 [SMILES: *OC[C@]12CCCN1[C@@H](COC(=O)N1CCOCC1)CC2 ; *OC[C@]12CCCN1[C@@H](COC(=O)N1CCCCC1)CC2 ; *OC[C@]12CCCN1[C@@H](COC(=O)N1CCCC1)CC2]
R₂ is selected from 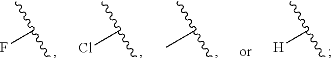
R₃ is selected from 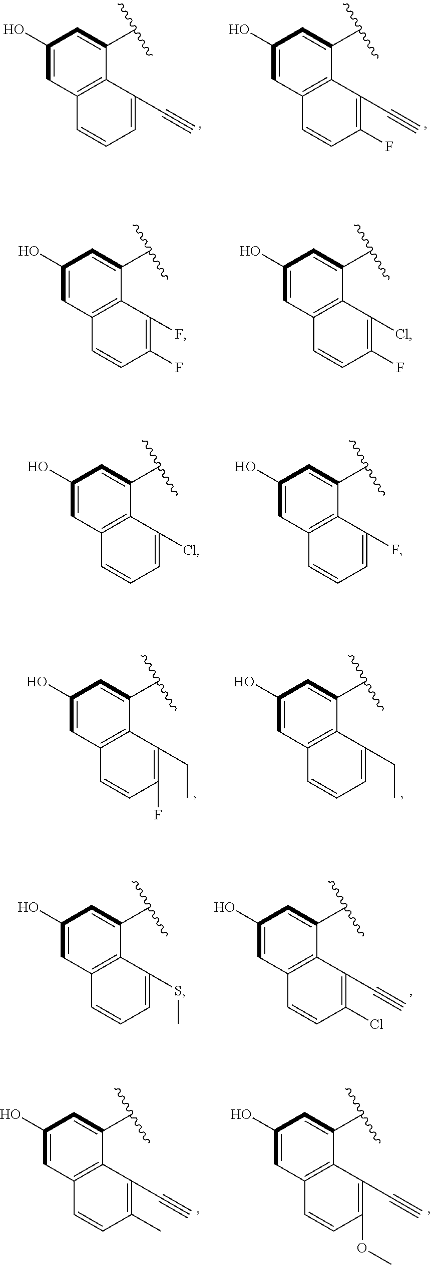 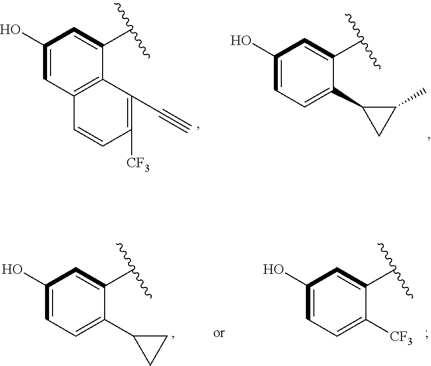
and
R₄ is selected from

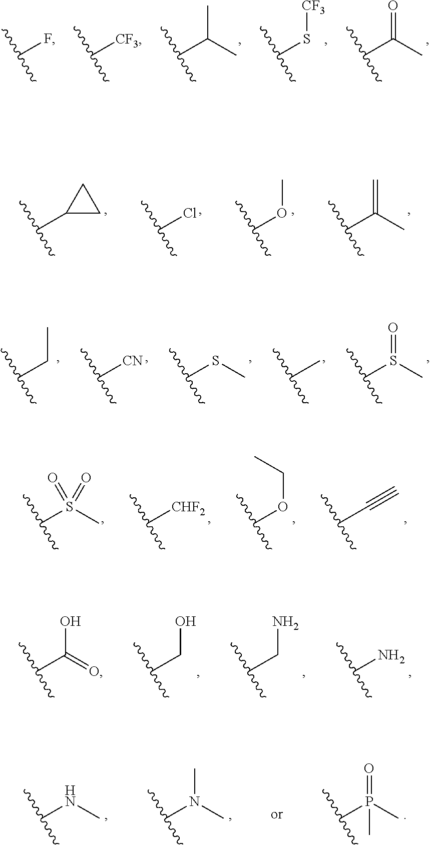

41. The compound of claim 27, wherein,
the compound is of formula (V-2): 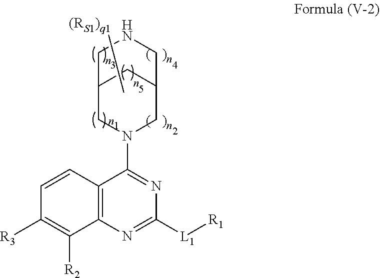
Wherein,
moiety of 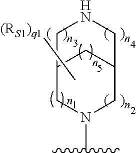
is selected from 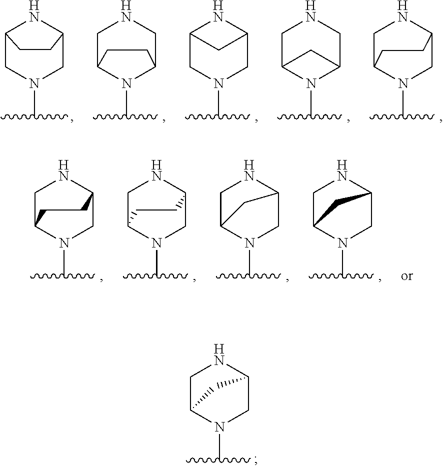
-L₁-R₁ is selected from 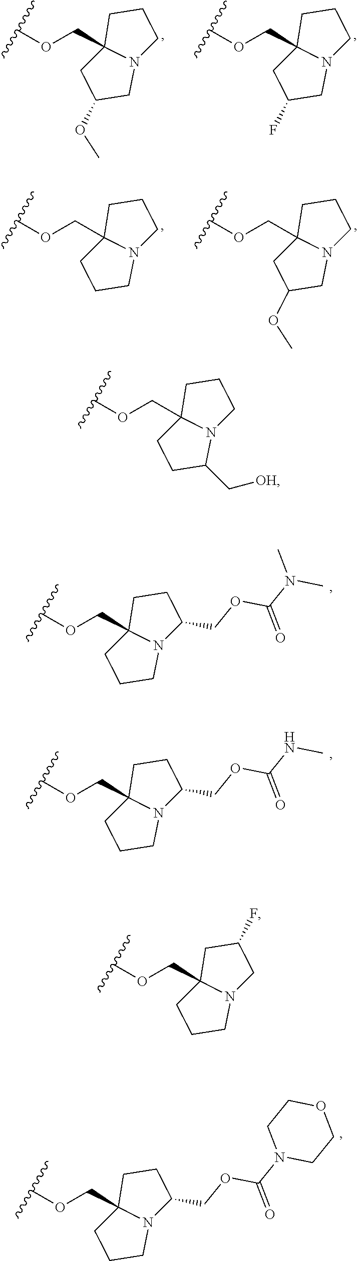 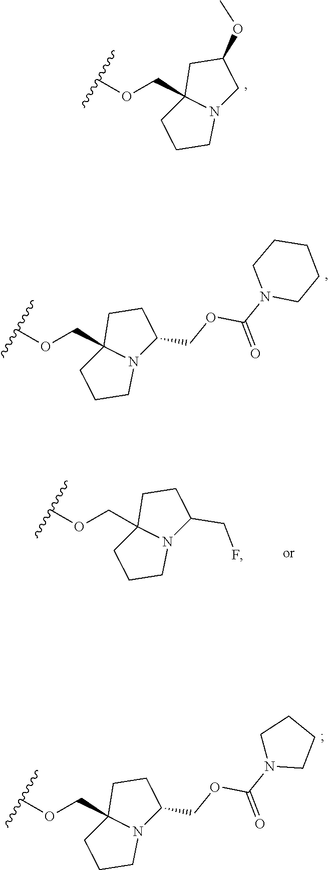 [SMILES: *OC[C@]12CCCN1C[C@H](OC)C2 ; *OC[C@]12CCCN1[C@@H](COC(=O)N1CCCCC1)CC2 ; *OCC12CCCN1C(CF)CC2 ; *OC[C@]12CCCN1[C@@H](COC(=O)N1CCCC1)CC2]
R₂ is selected from 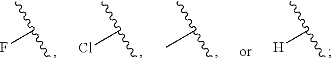
and
R₃ is selected from

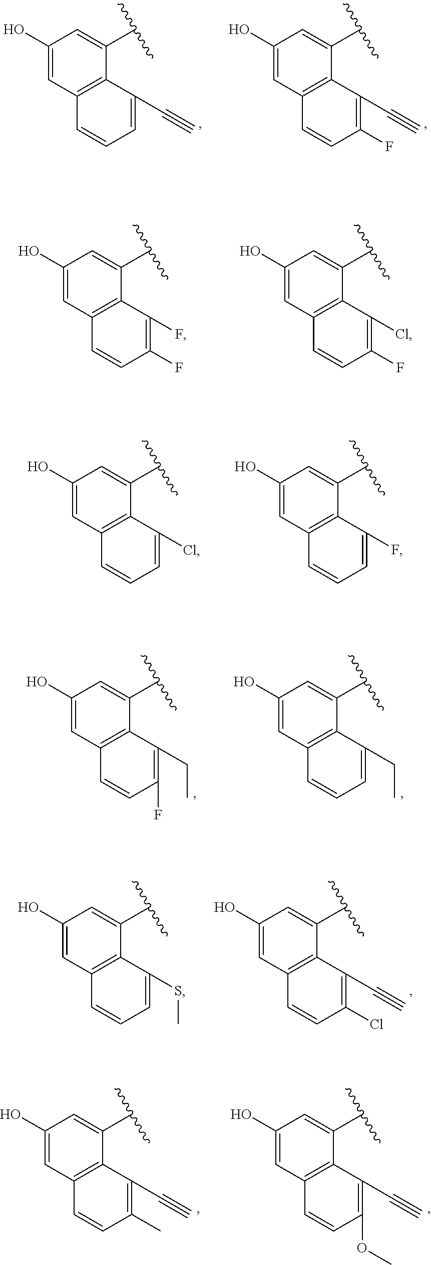

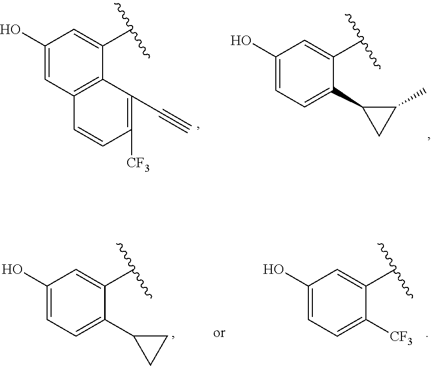 [SMILES: *c1cc(O)cc2ccc(C(F)(F)F)c(C#C)c12 ; *c1cc(O)ccc1[C@@H]1C[C@H]1C ; *c1cc(O)ccc1C1CC1 ; *c1cc(O)ccc1C(F)(F)F]

42. The compound of claim 27, wherein,
the compound is of formula (V-3): 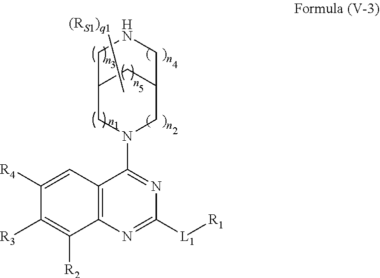
Wherein,
moiety of 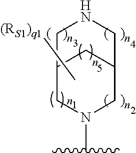
is selected from 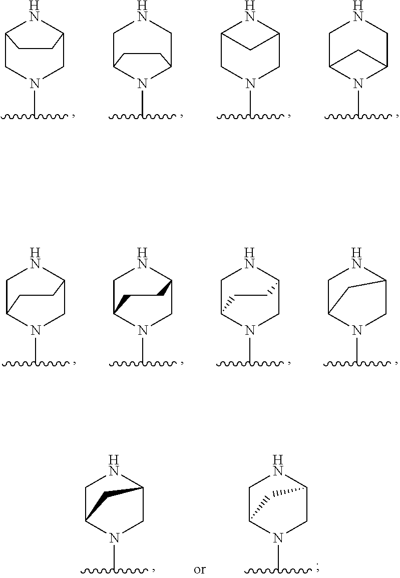
-L₁-R₁ is selected from 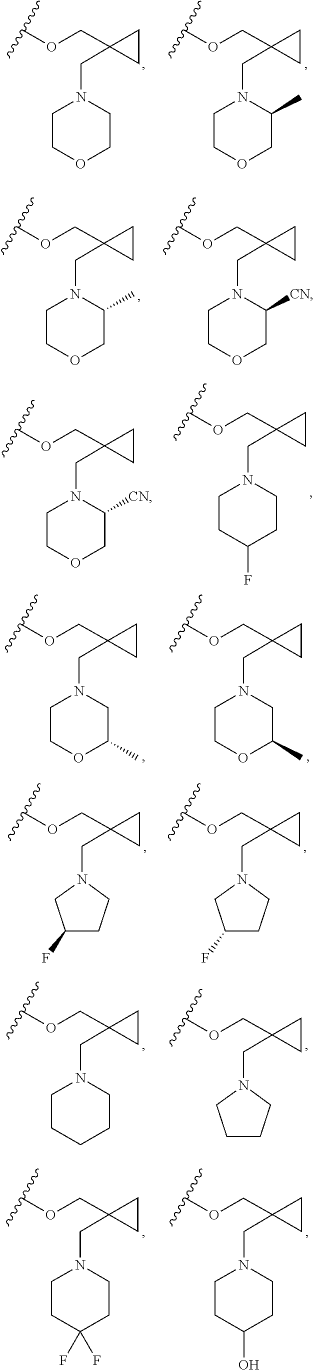 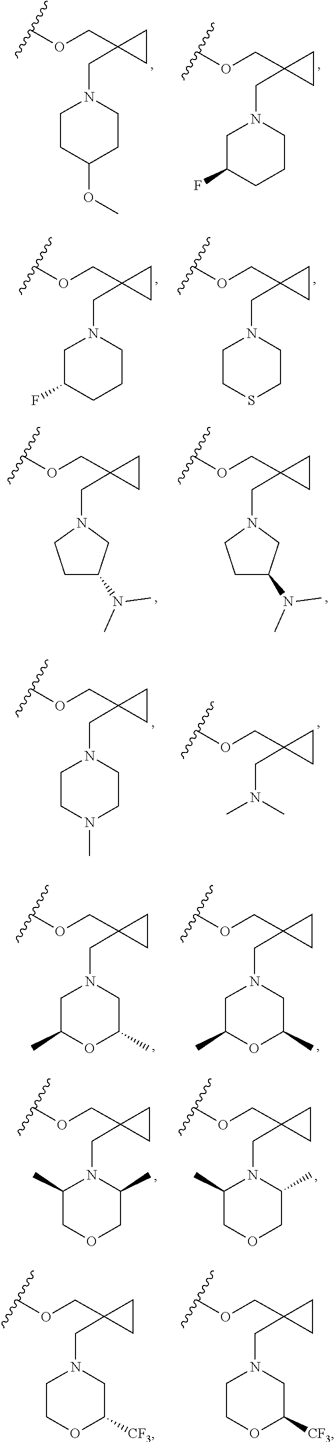 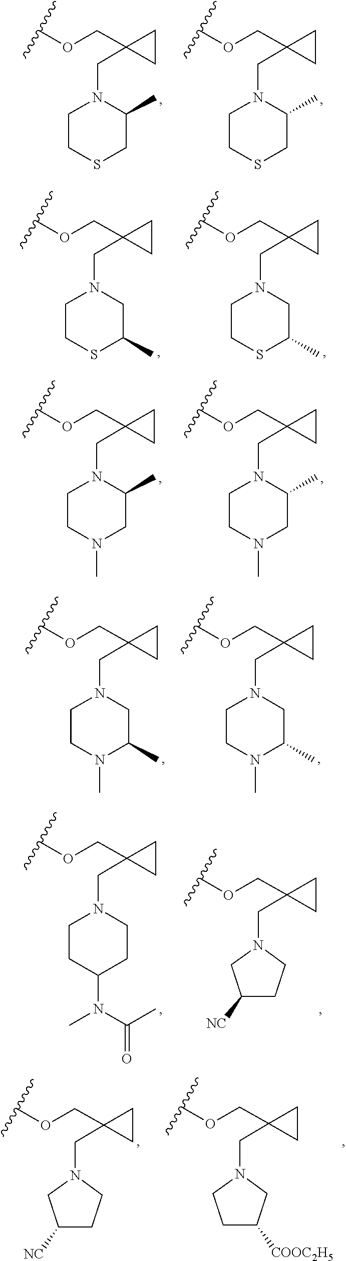 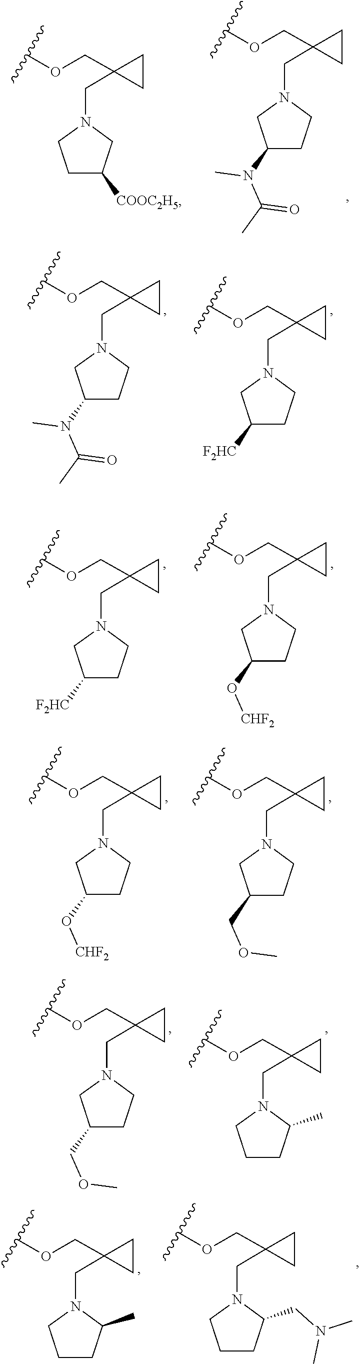 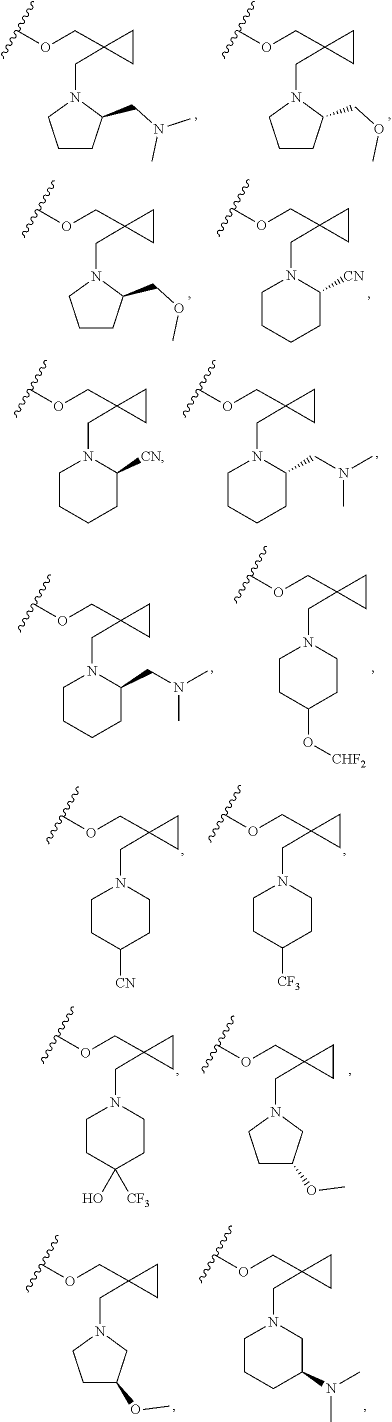 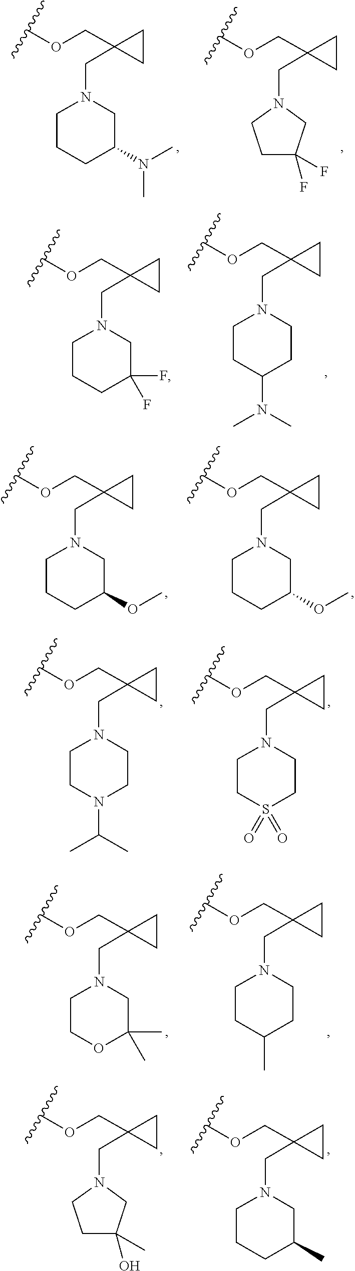 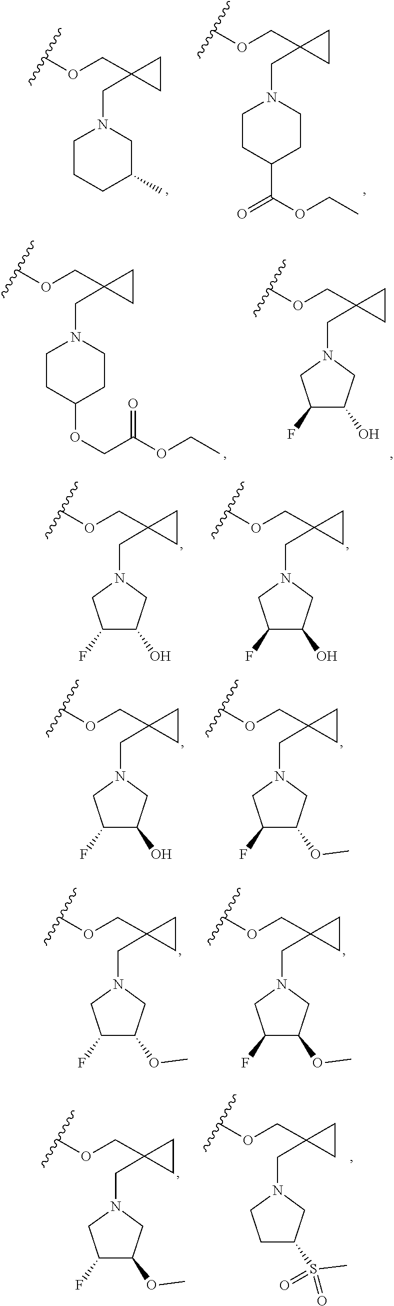 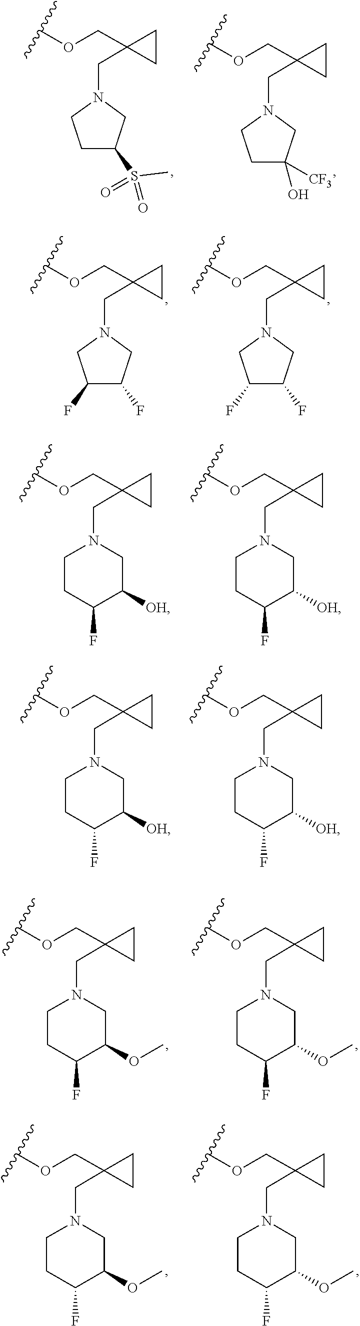 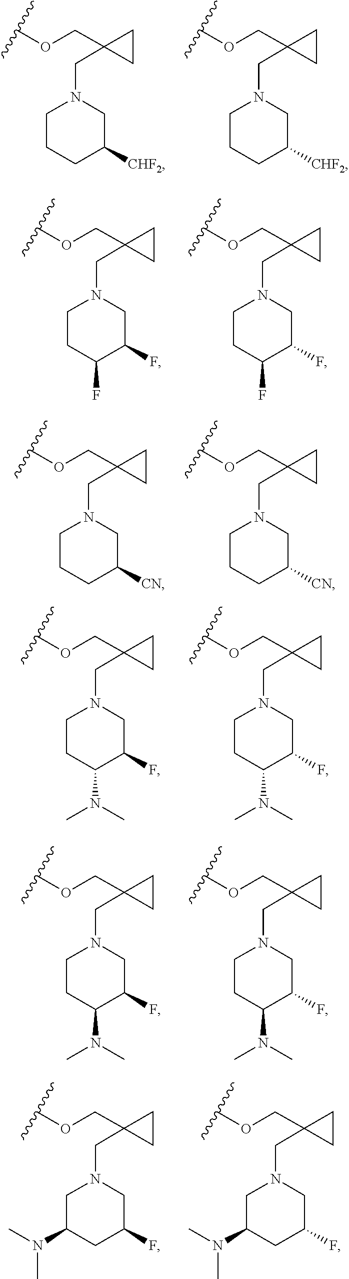 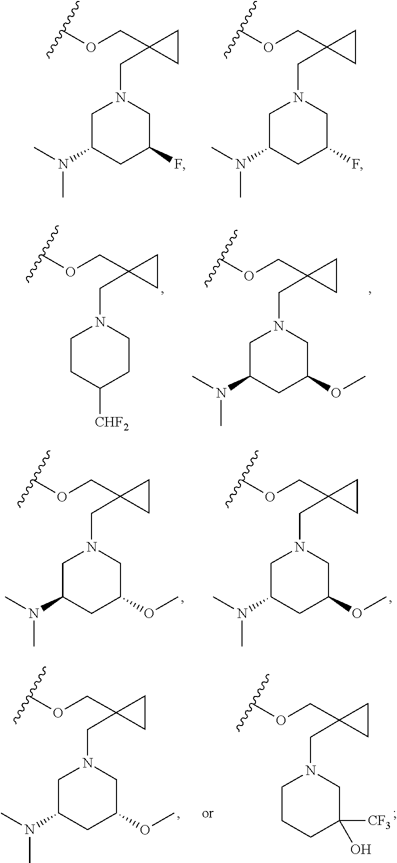
R₂ is selected from 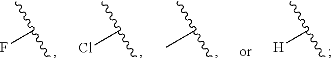
R₃ is selected from 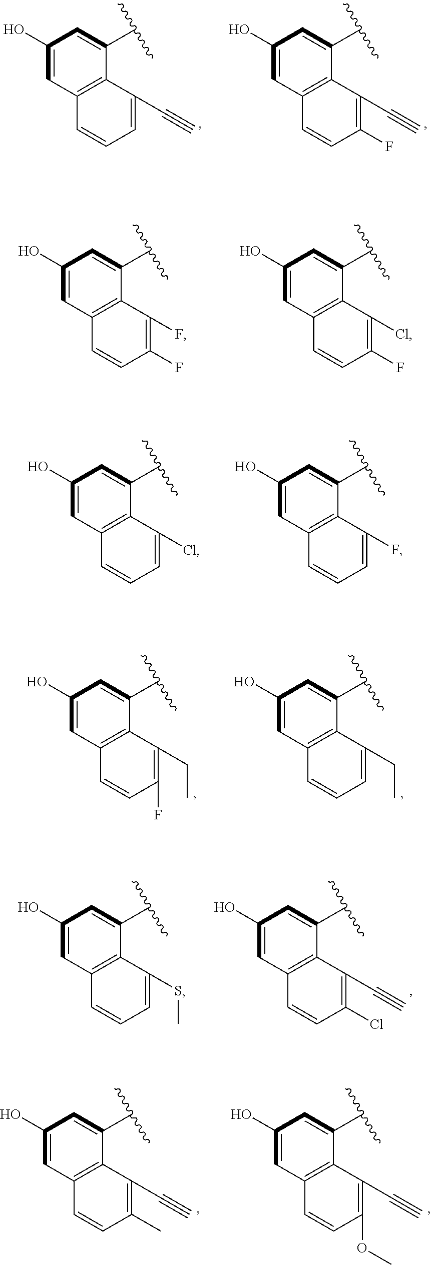 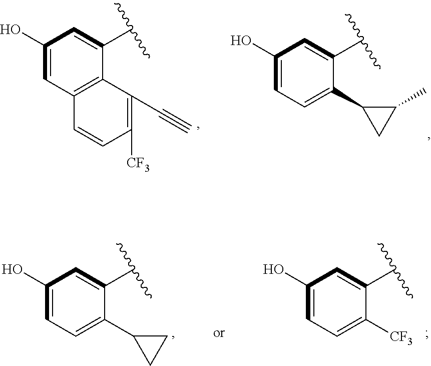
R₄ is selected from

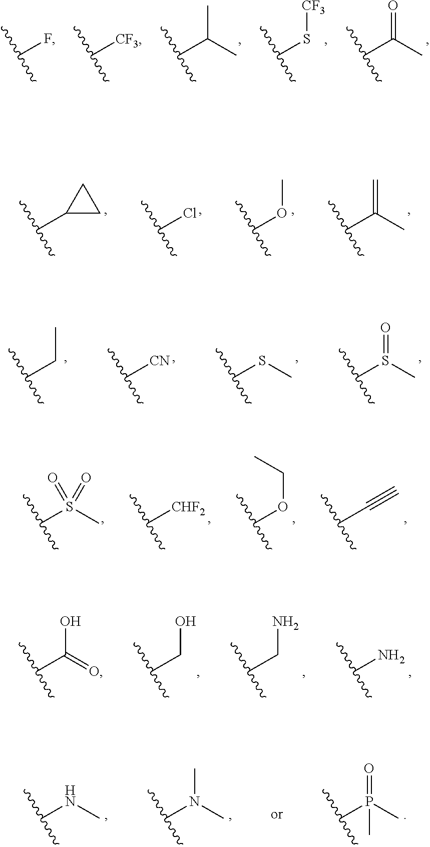

43. The compound of claim 27, wherein,
the compound is of formula (V-4): 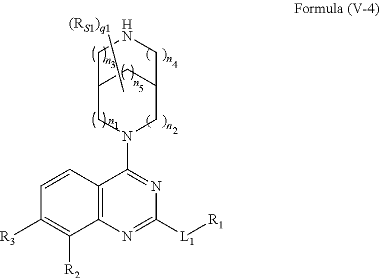
Wherein,
moiety of 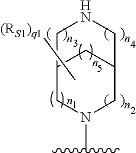
is selected from 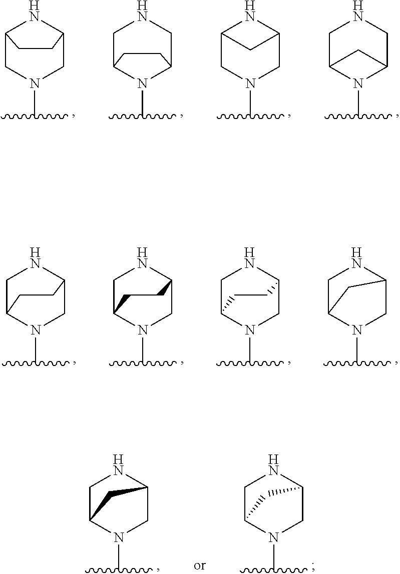
-L₁-R₁ is selected from 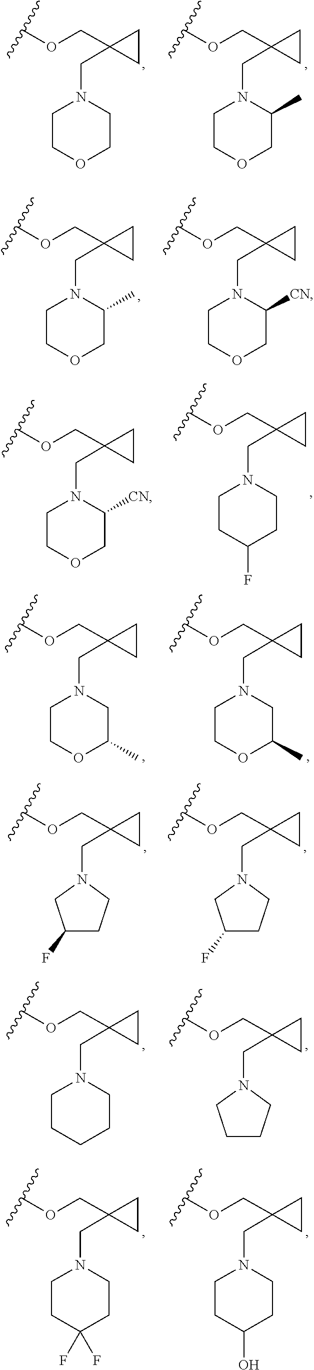 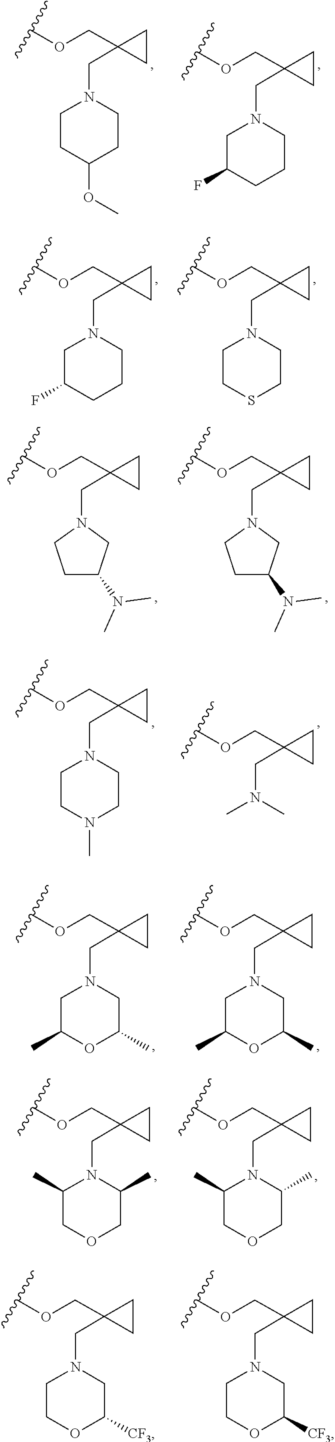 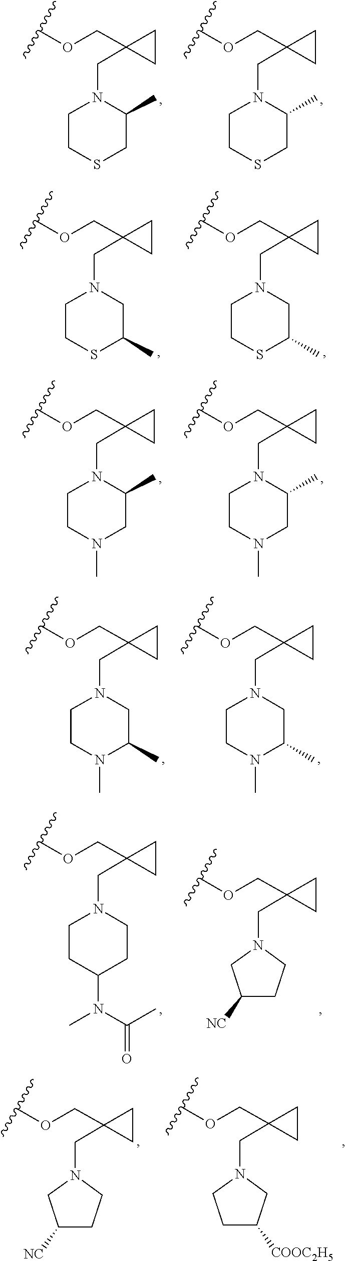 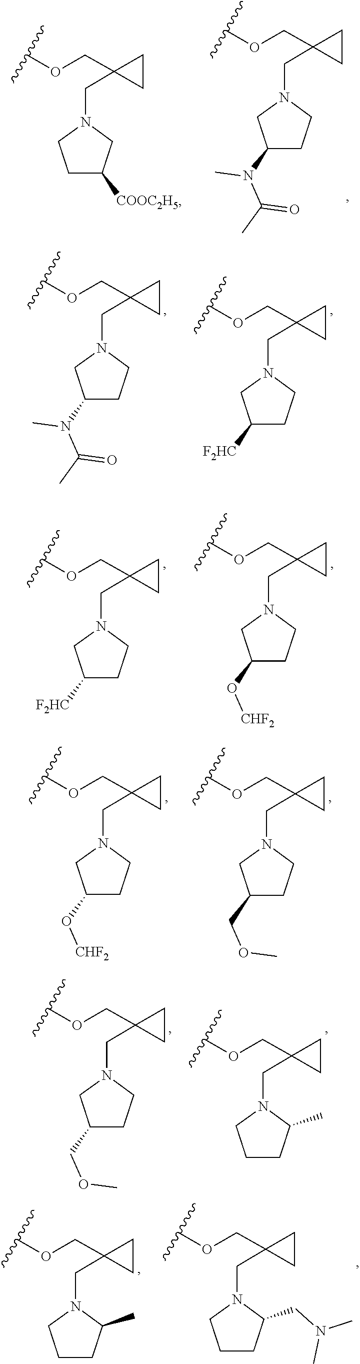 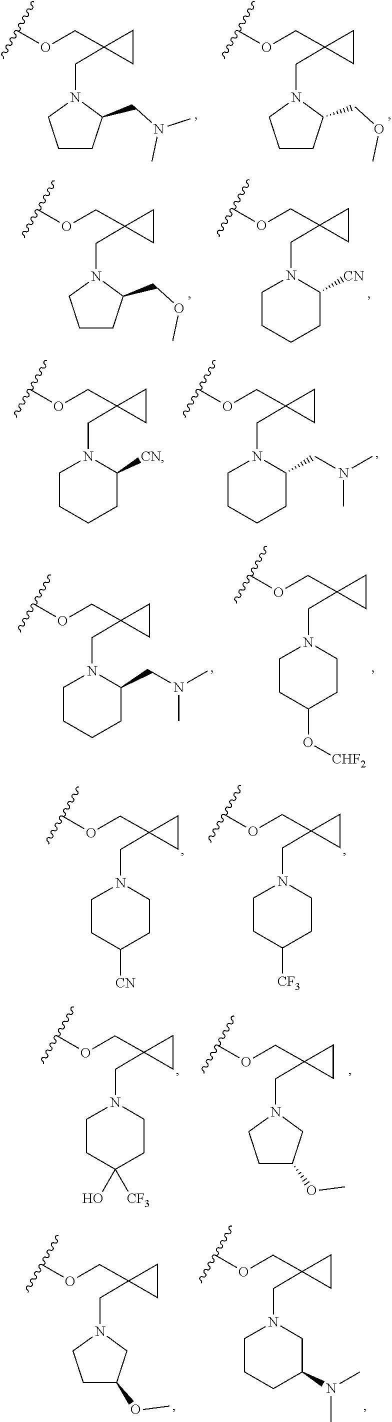 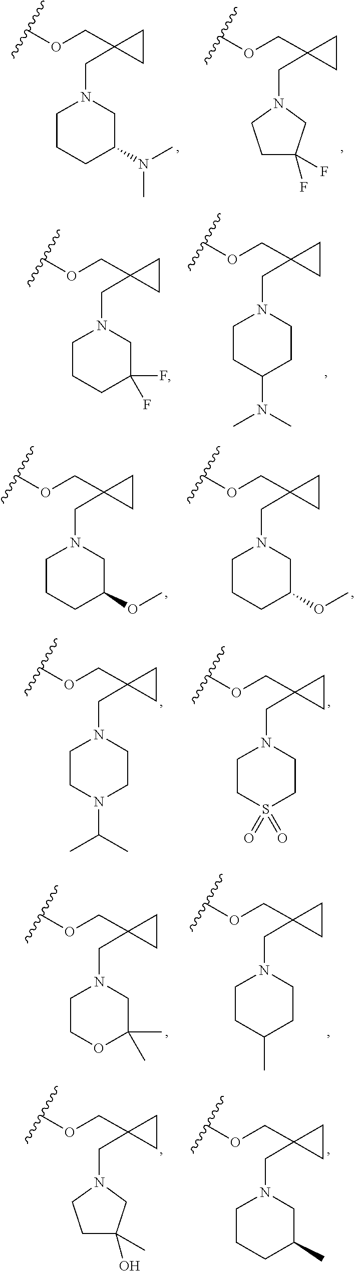 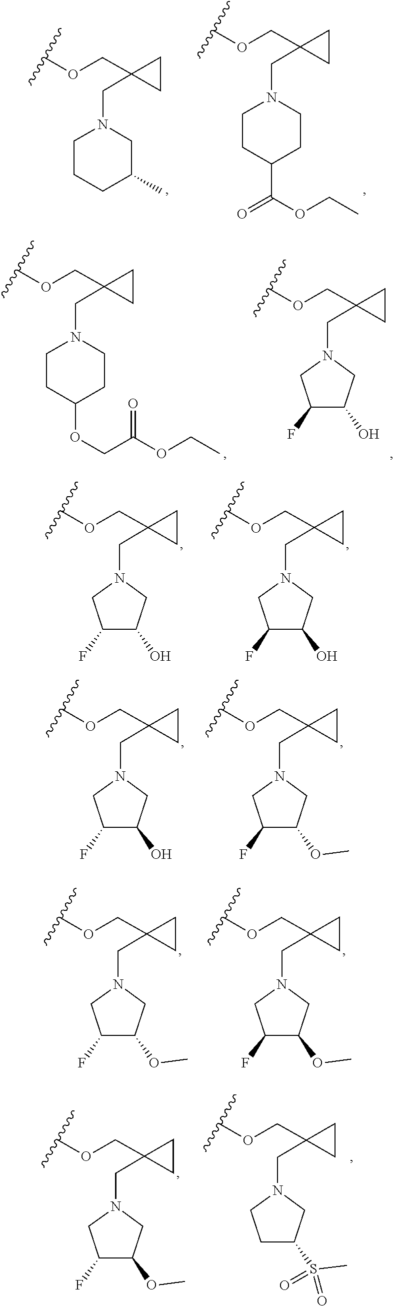 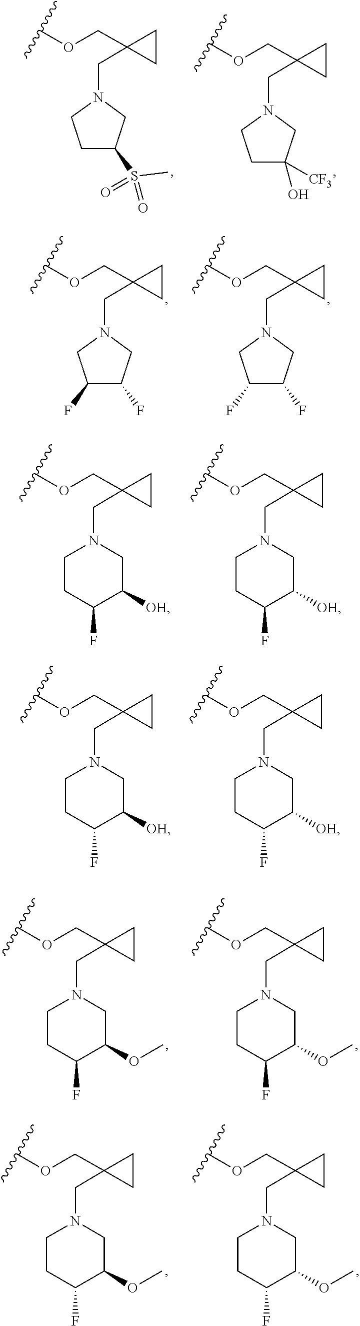 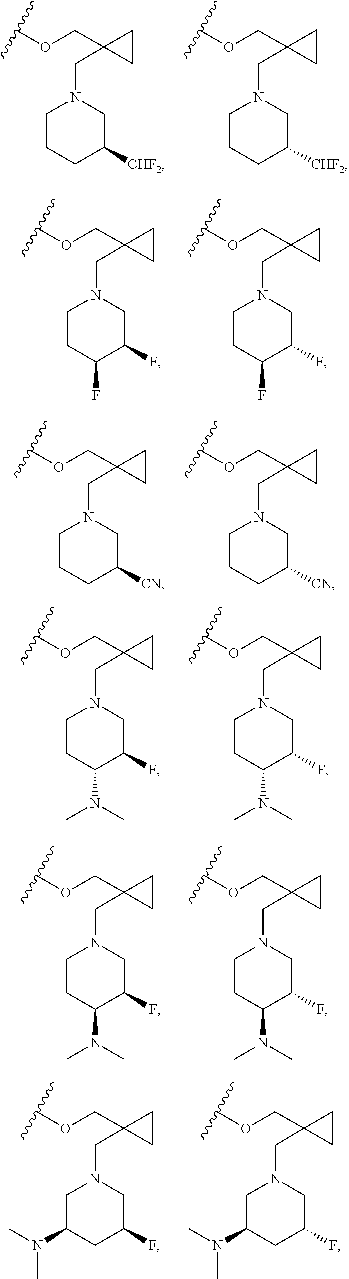 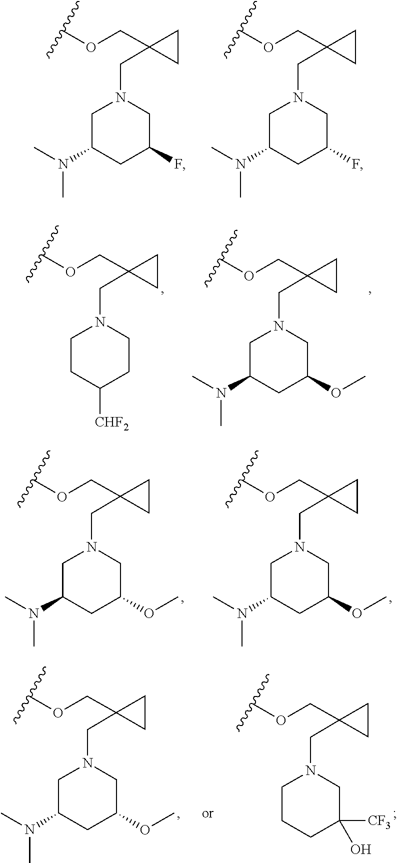
R₂ is selected from 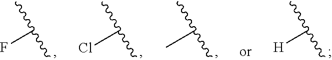
and
R₃ is selected from

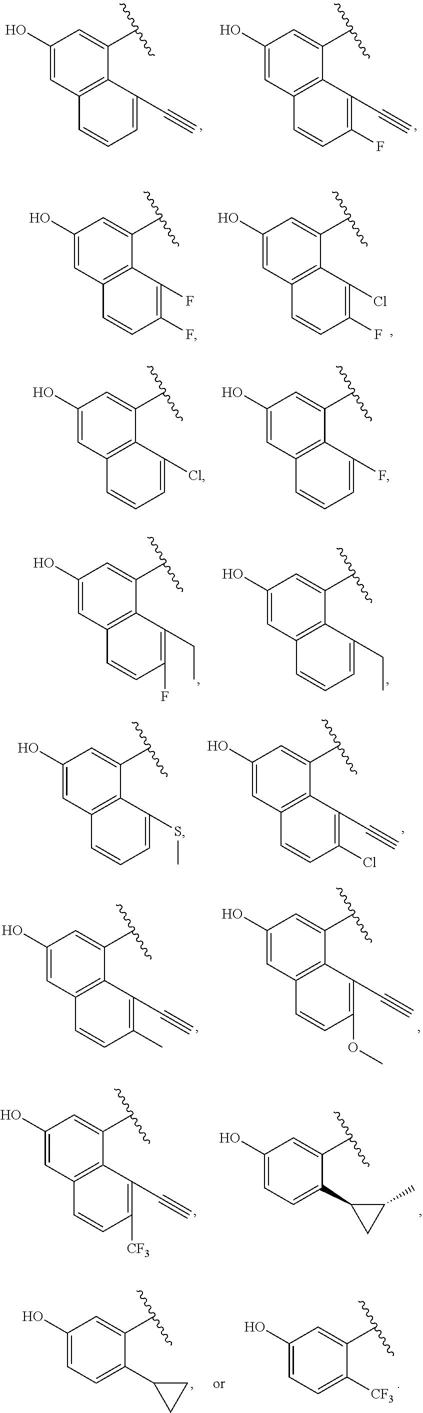

44. The compound of claim 27, wherein,
the compound is selected from:

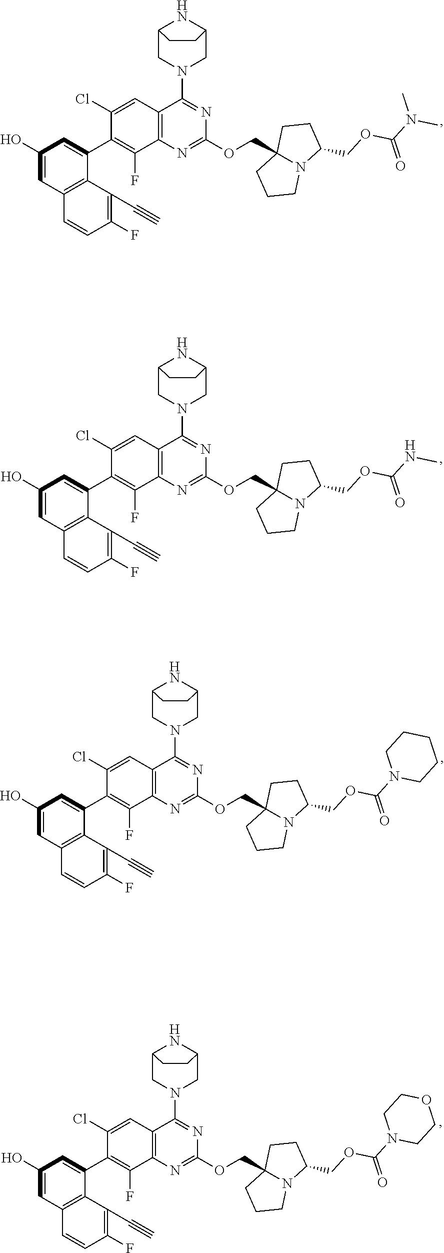

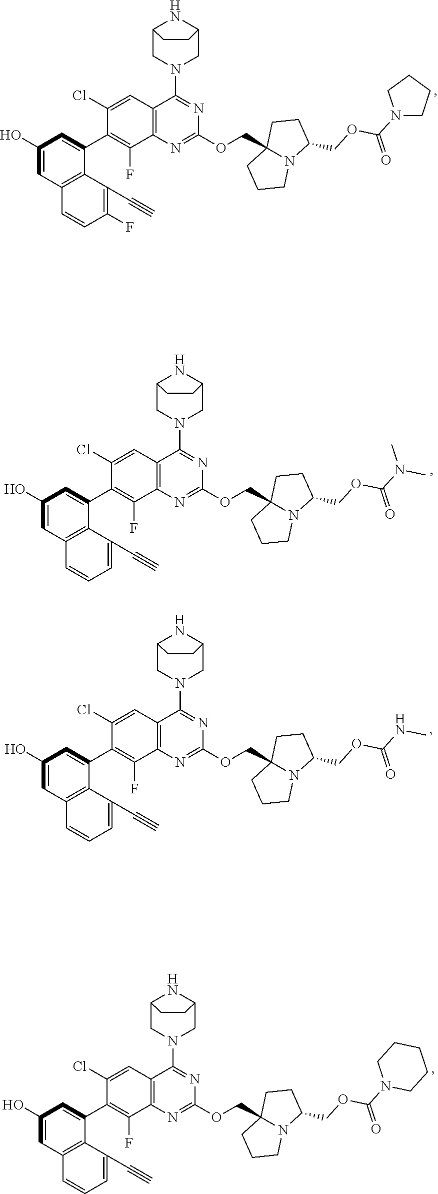

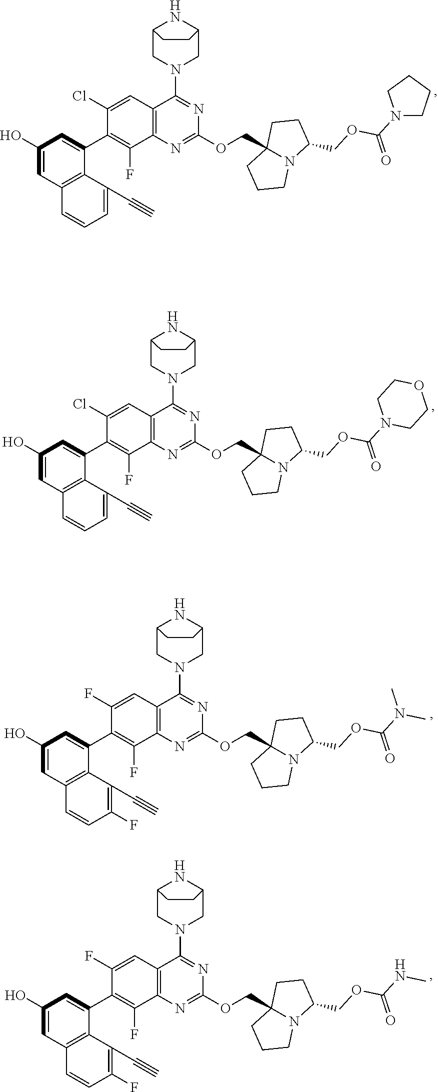

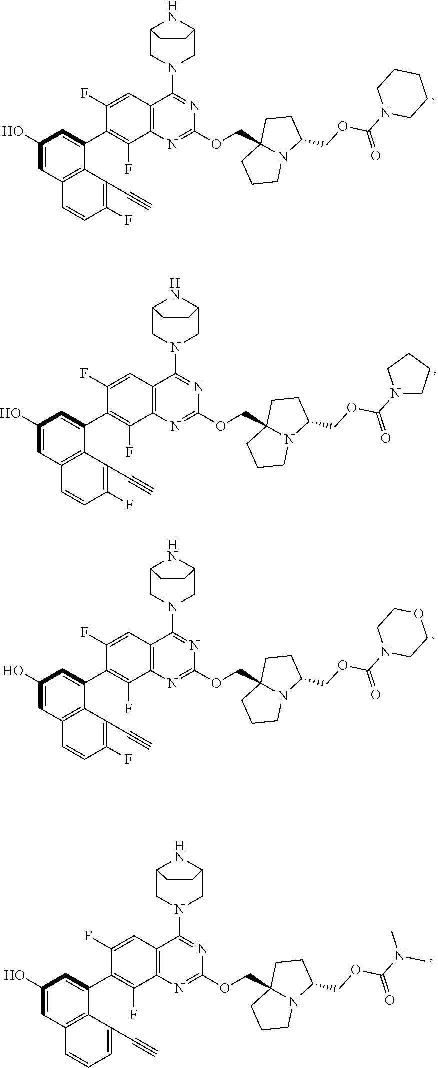

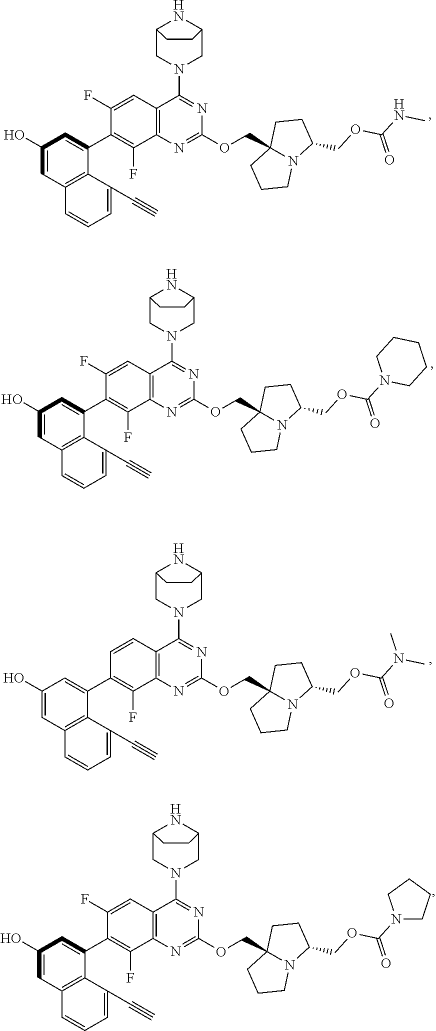

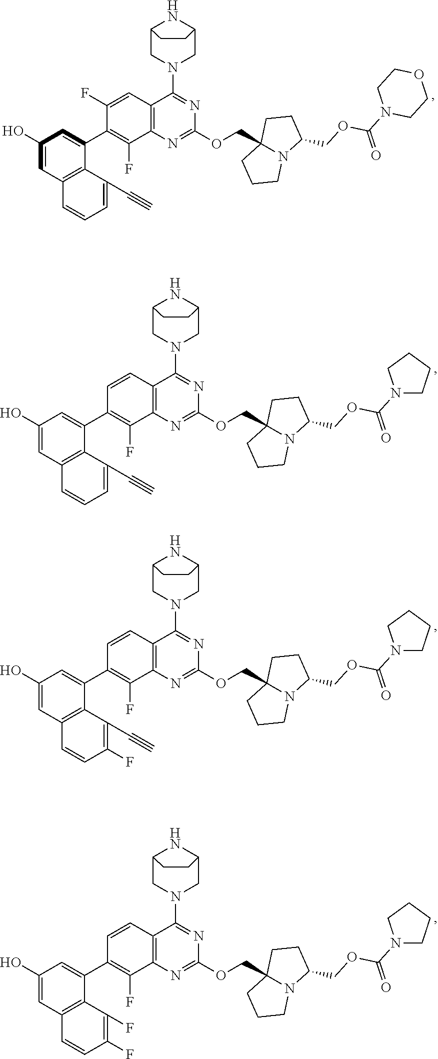

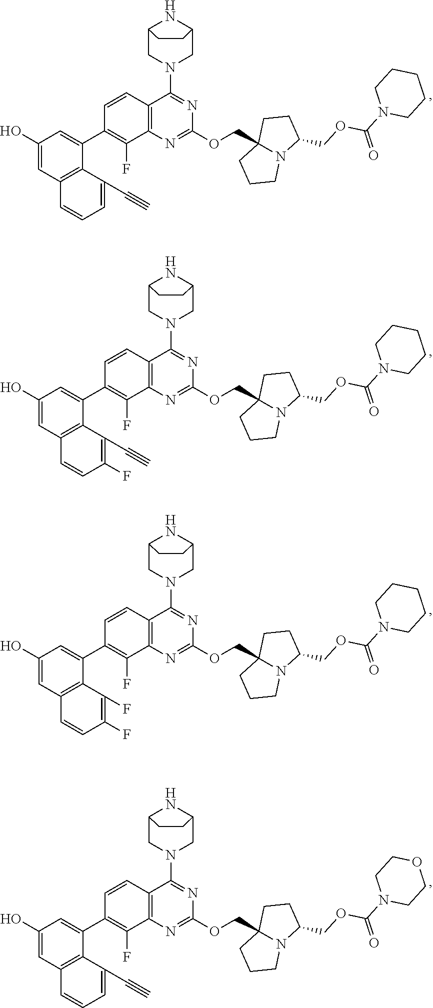 [SMILES: C#Cc1cccc2cc(O)cc(-c3ccc4c(N5CC6CCC(C5)N6)nc(OC[C@]56CCCN5[C@@H](COC(=O)N5CCCCC5)CC6)nc4c3F)c12 ; C#Cc1c(F)ccc2cc(O)cc(-c3ccc4c(N5CC6CCC(C5)N6)nc(OC[C@]56CCCN5[C@@H](COC(=O)N5CCCCC5)CC6)nc4c3F)c12 ; O=C(OC[C@H]1CC[C@@]2(COc3nc(N4CC5CCC(C4)N5)c4ccc(-c5cc(O)cc6ccc(F)c(F)c56)c(F)c4n3)CCCN12)N1CCCCC1 ; C#Cc1cccc2cc(O)cc(-c3ccc4c(N5CC6CCC(C5)N6)nc(OC[C@]56CCCN5[C@@H](COC(=O)N5CCOCC5)CC6)nc4c3F)c12]

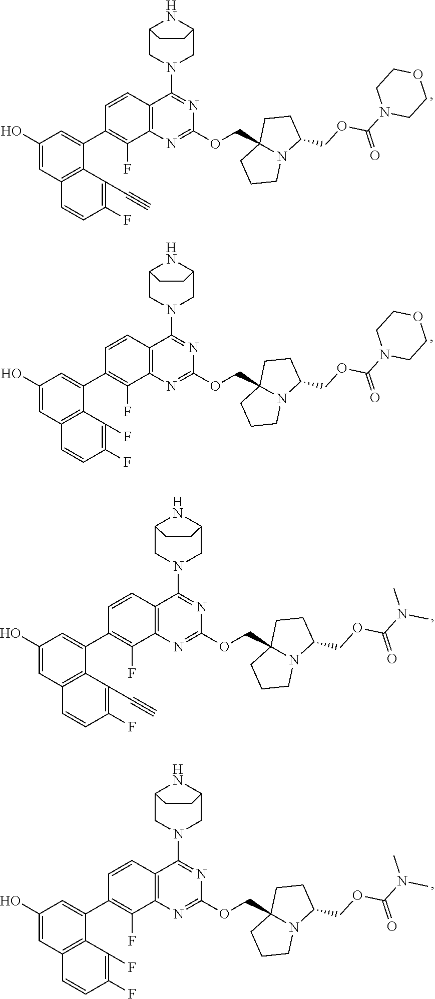 [SMILES: C#Cc1c(F)ccc2cc(O)cc(-c3ccc4c(N5CC6CCC(C5)N6)nc(OC[C@]56CCCN5[C@@H](COC(=O)N5CCOCC5)CC6)nc4c3F)c12 ; O=C(OC[C@H]1CC[C@@]2(COc3nc(N4CC5CCC(C4)N5)c4ccc(-c5cc(O)cc6ccc(F)c(F)c56)c(F)c4n3)CCCN12)N1CCOCC1 ; C#Cc1c(F)ccc2cc(O)cc(-c3ccc4c(N5CC6CCC(C5)N6)nc(OC[C@]56CCCN5[C@@H](COC(=O)N(C)C)CC6)nc4c3F)c12 ; CN(C)C(=O)OC[C@H]1CC[C@@]2(COc3nc(N4CC5CCC(C4)N5)c4ccc(-c5cc(O)cc6ccc(F)c(F)c56)c(F)c4n3)CCCN12]

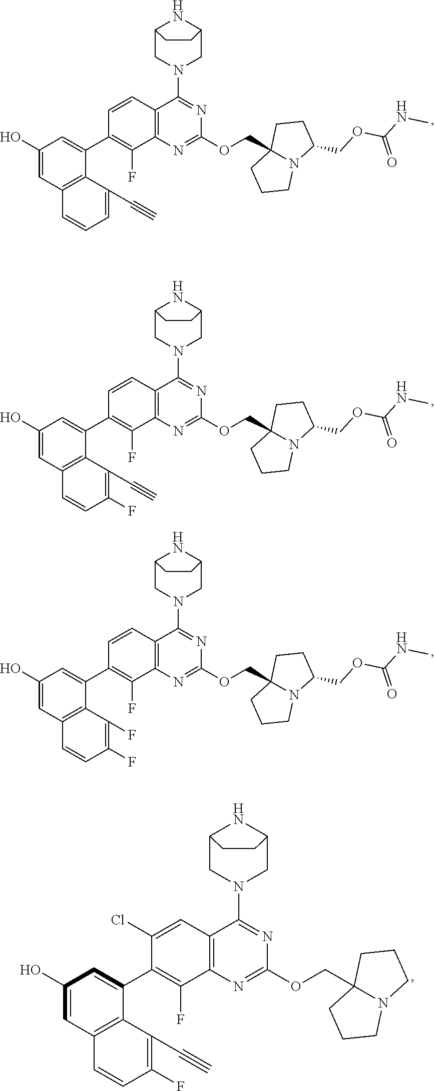

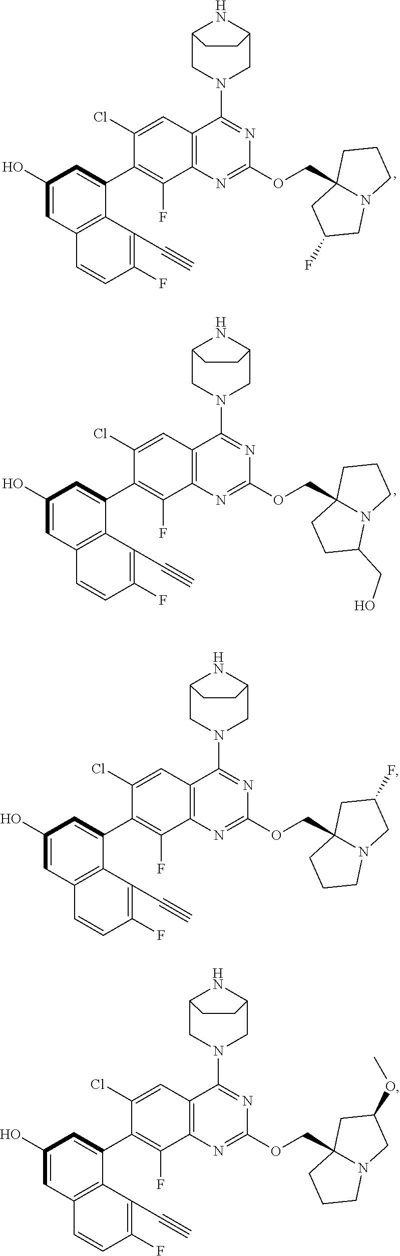

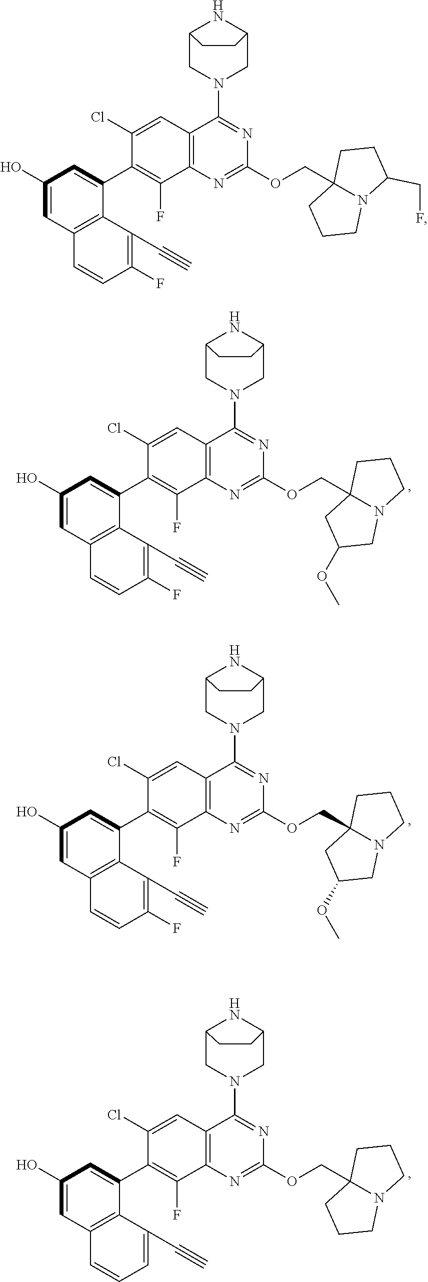

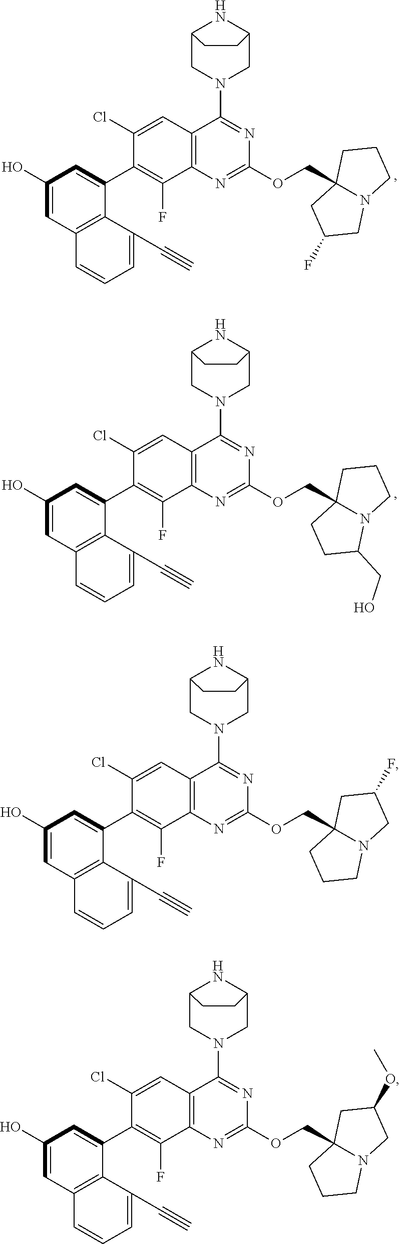

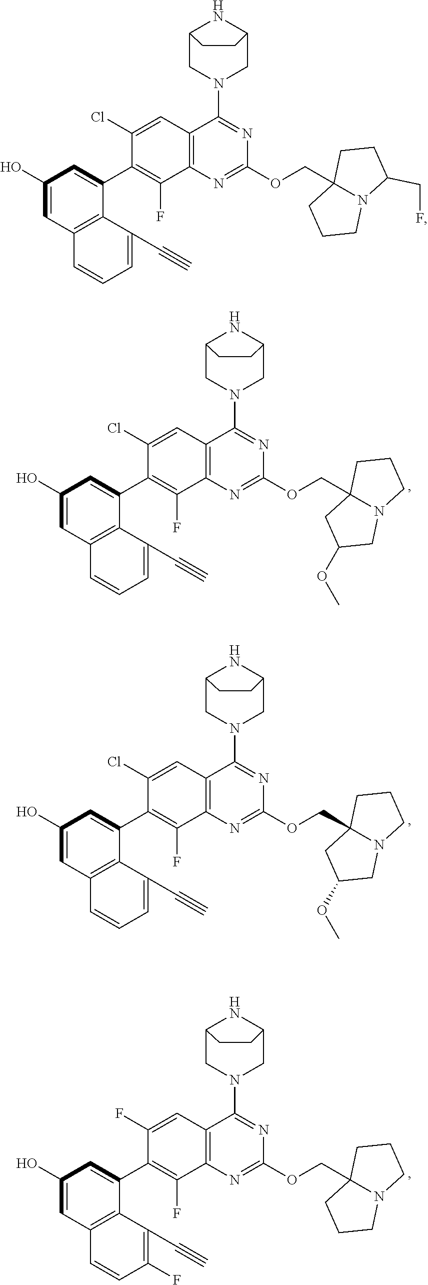

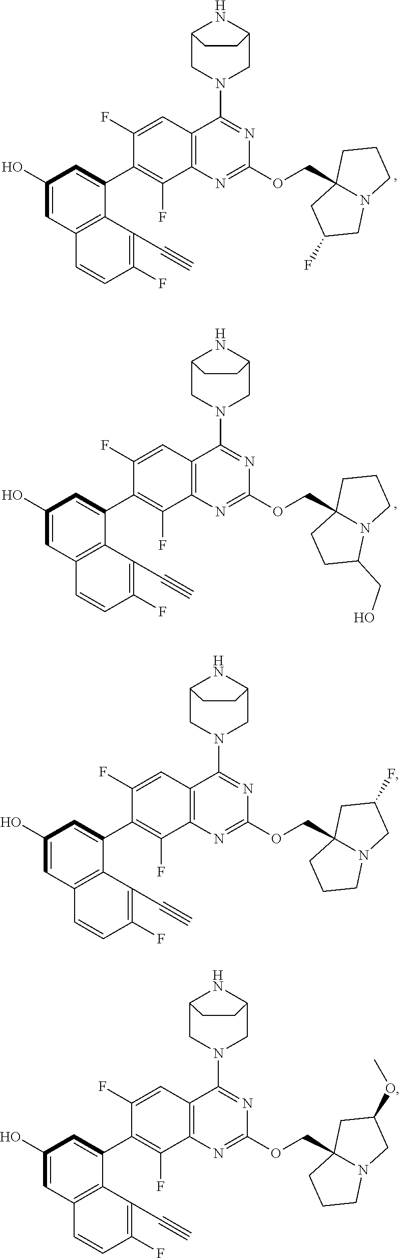

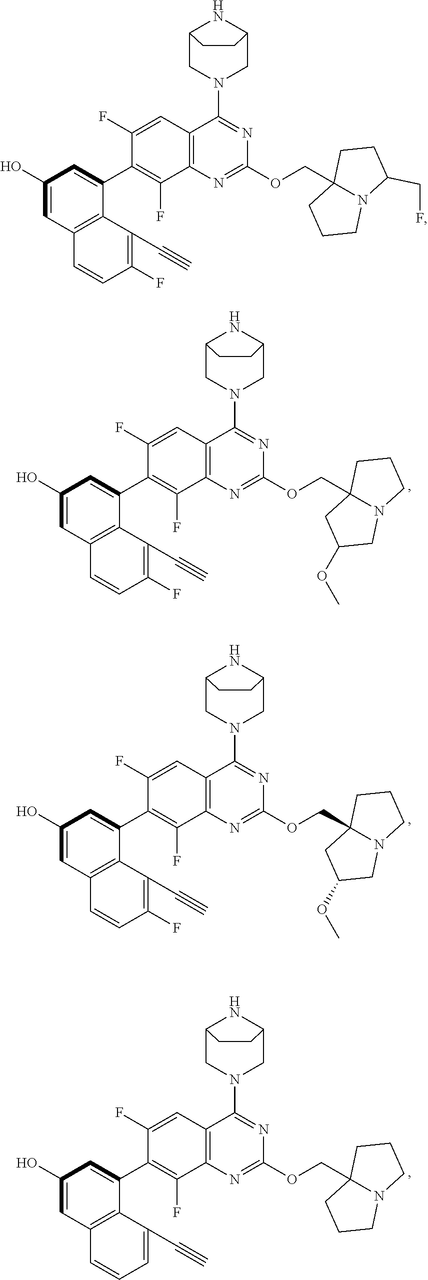

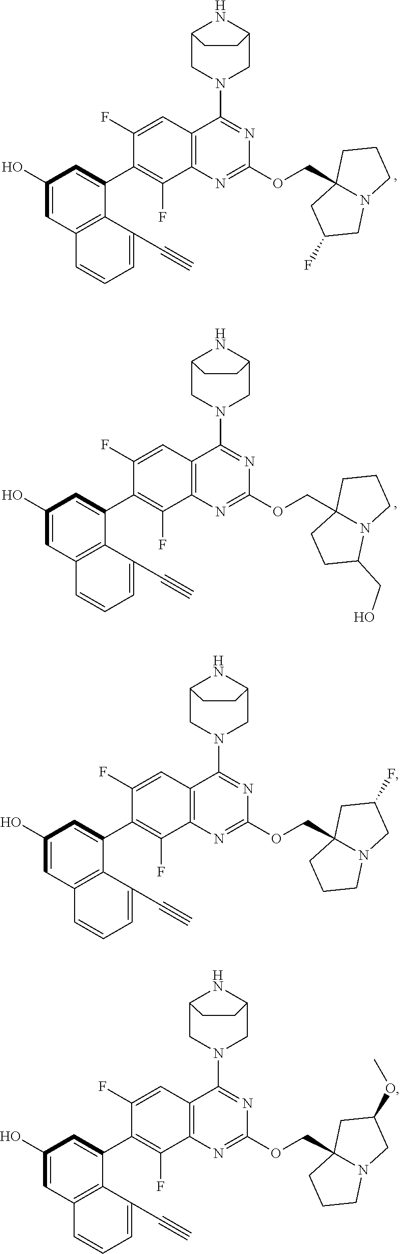

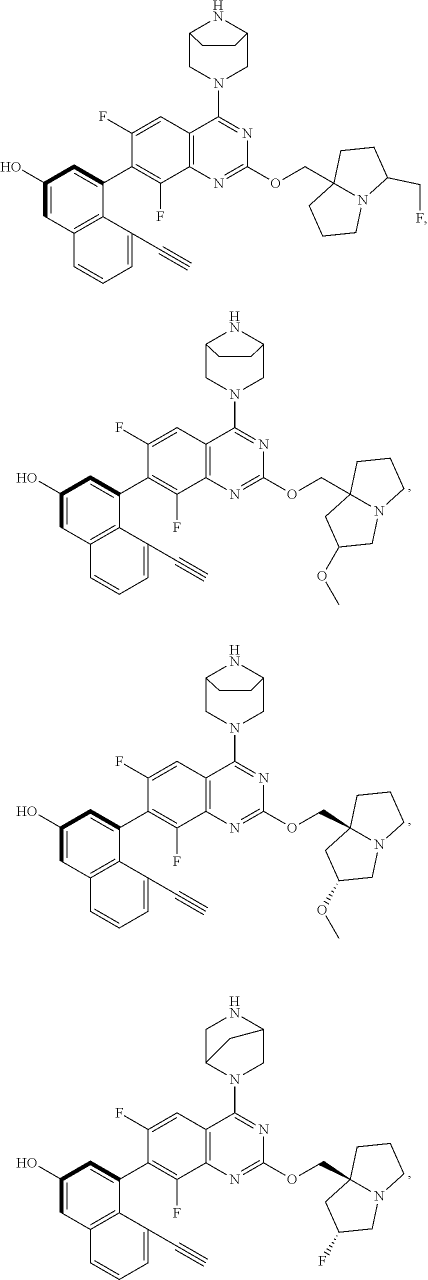

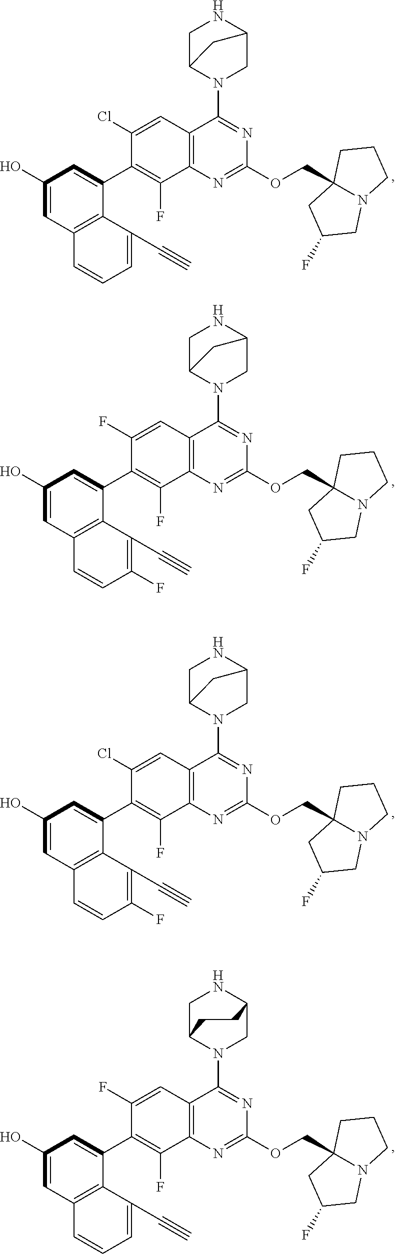

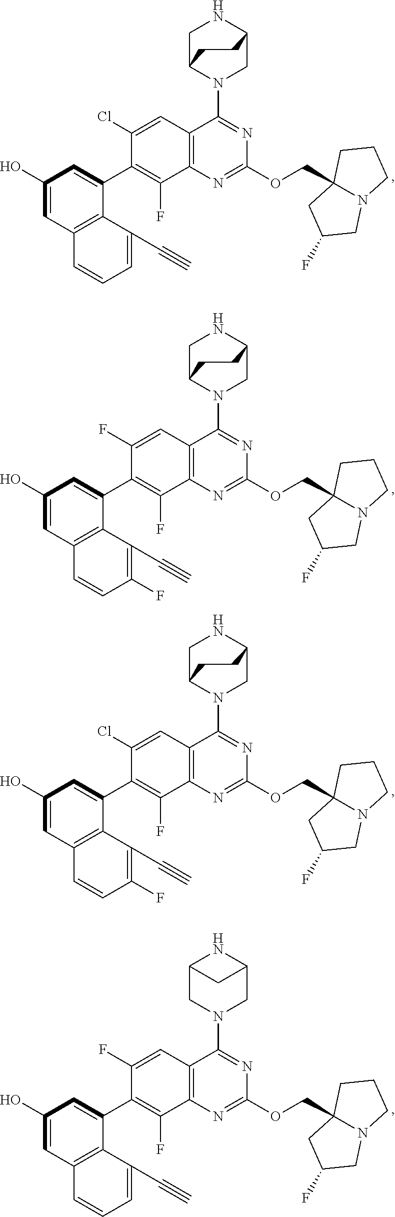

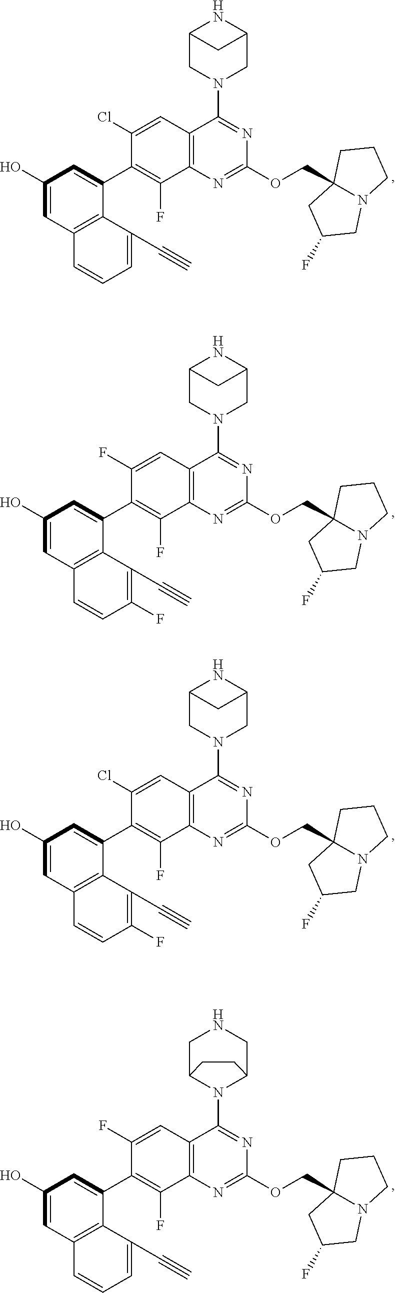

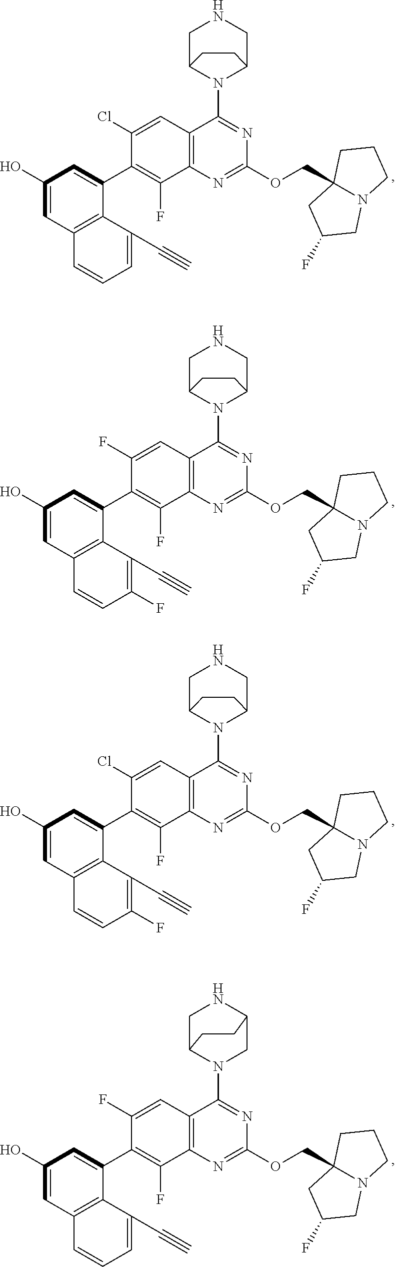

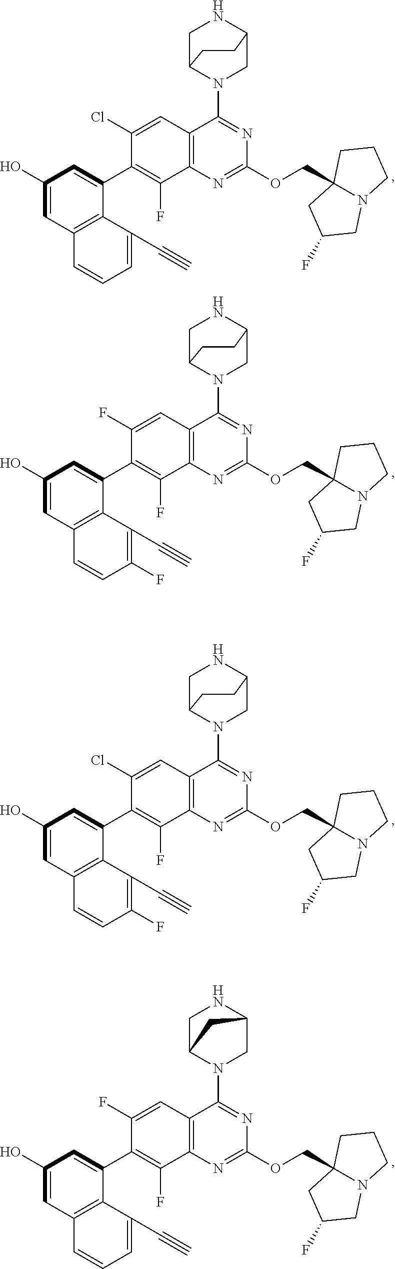

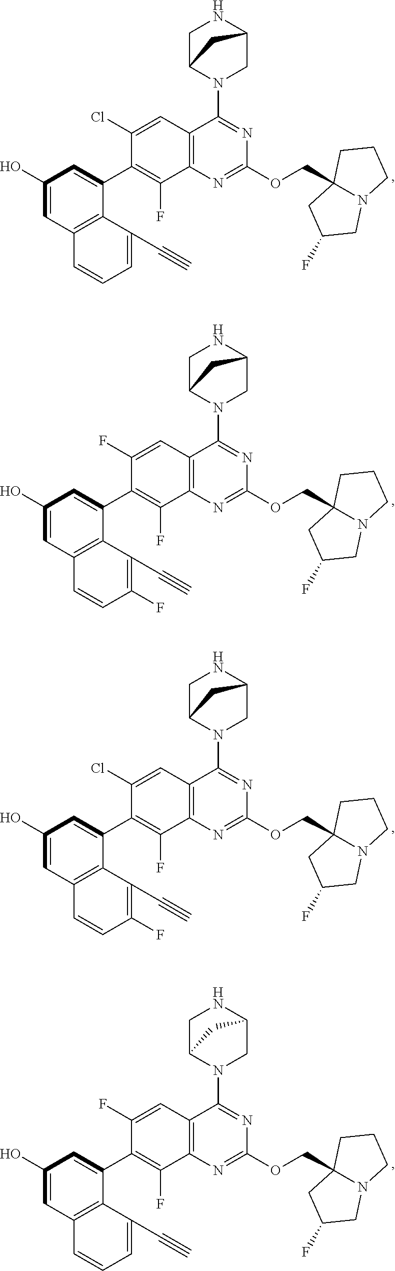

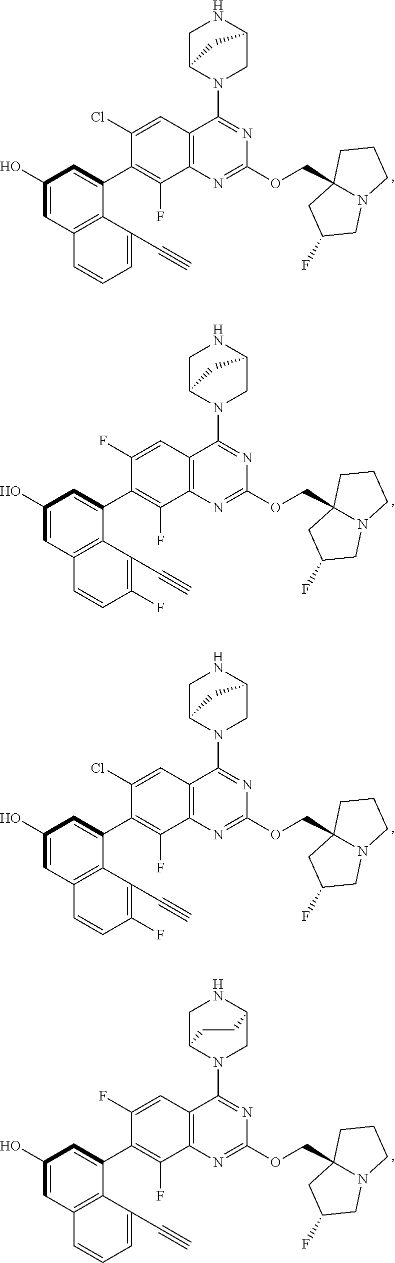

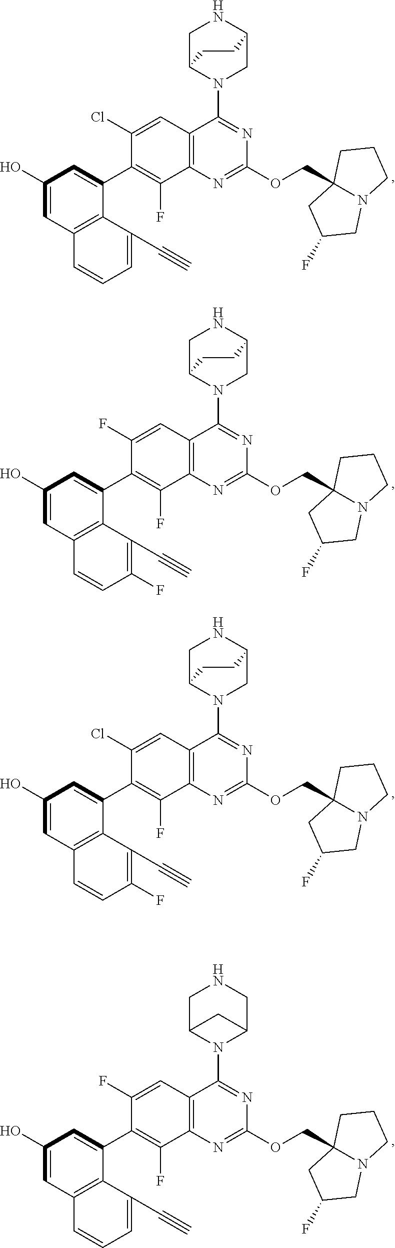

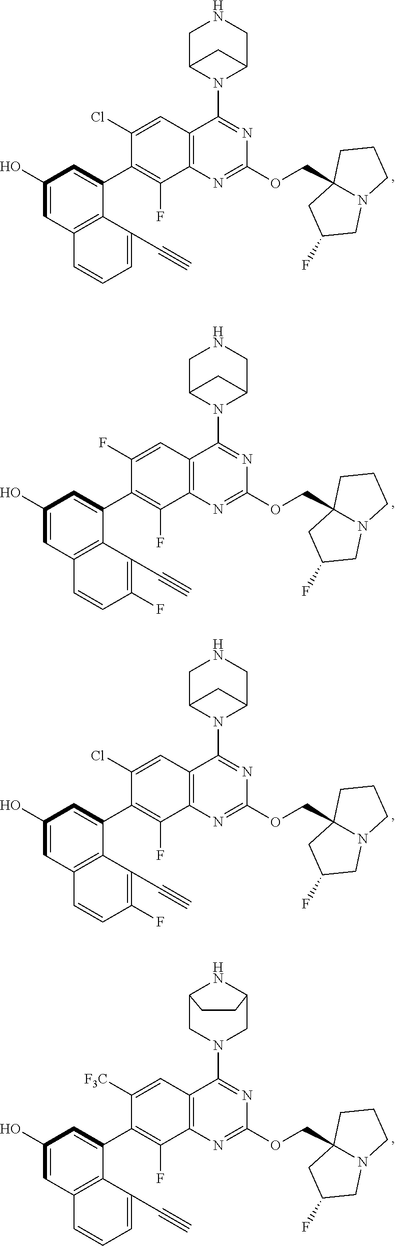

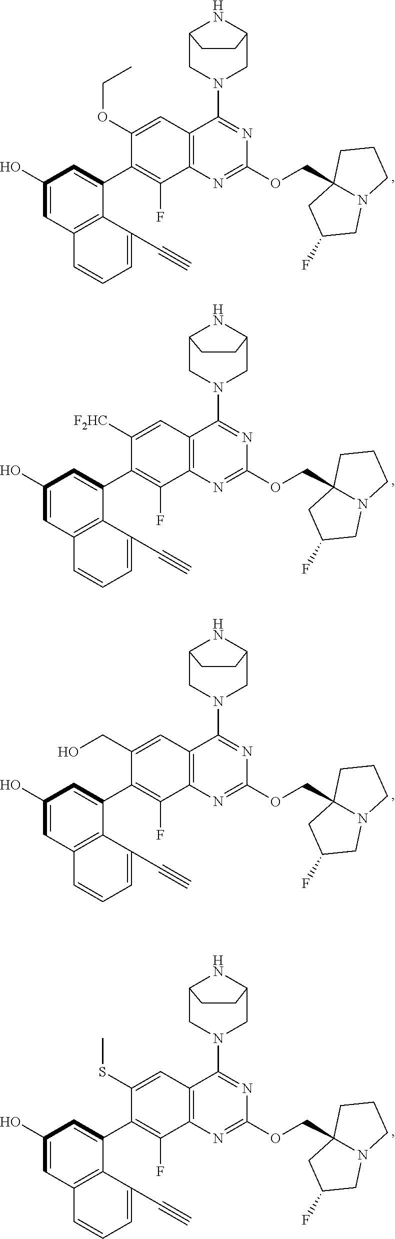

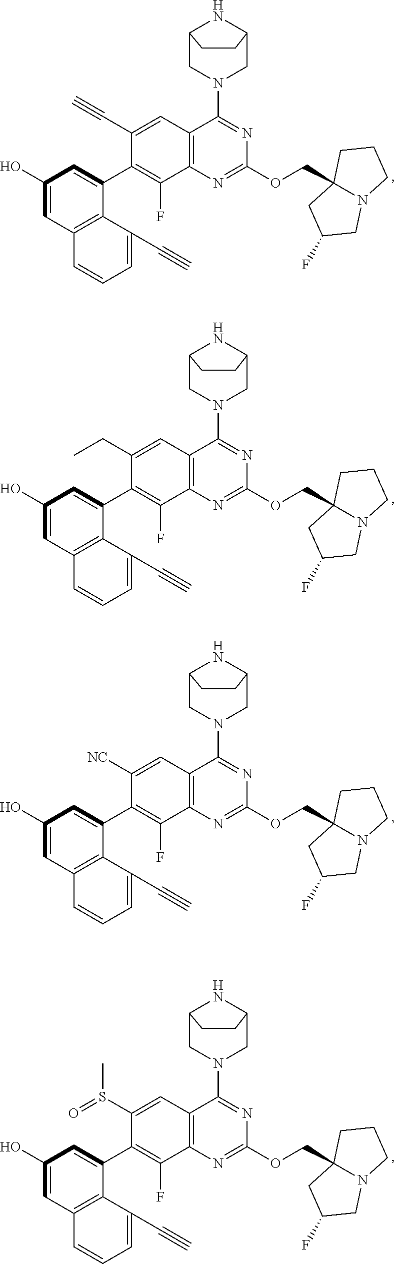

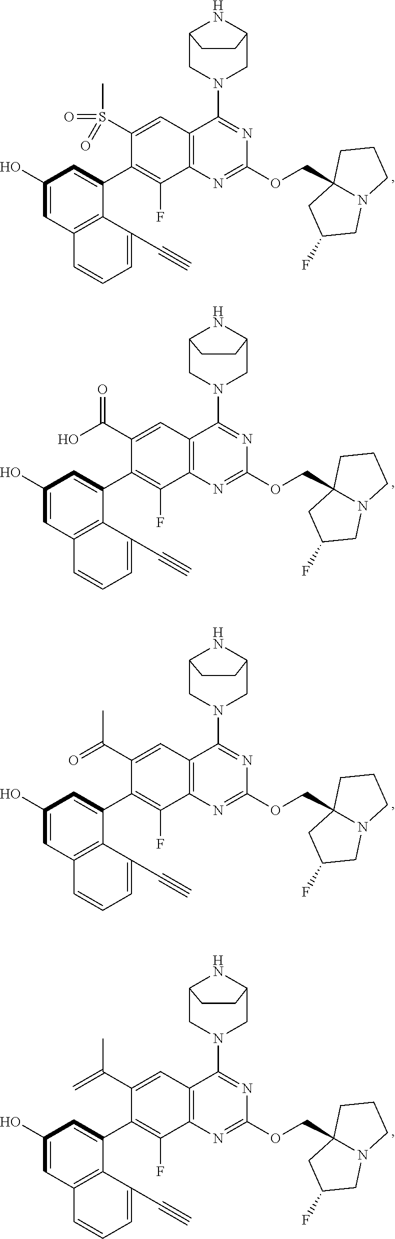

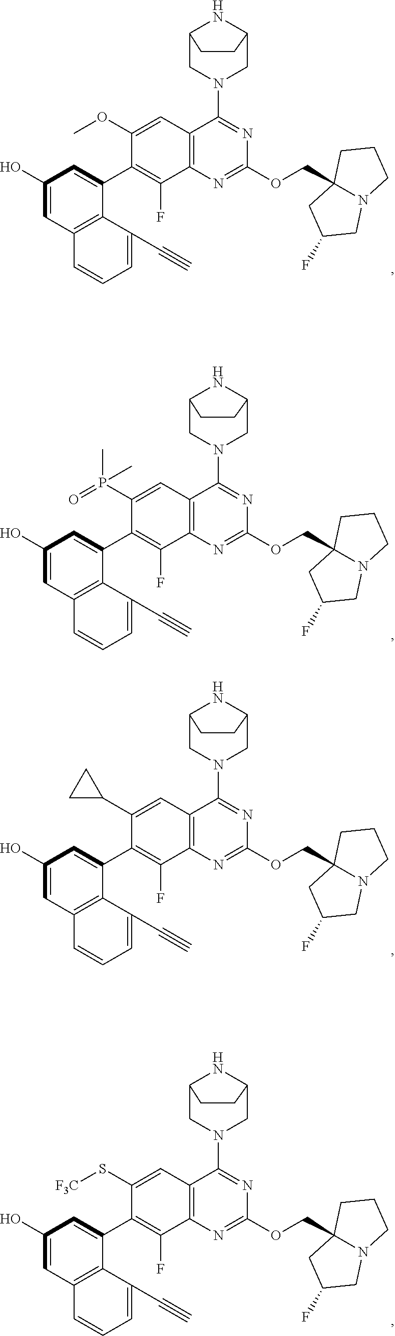

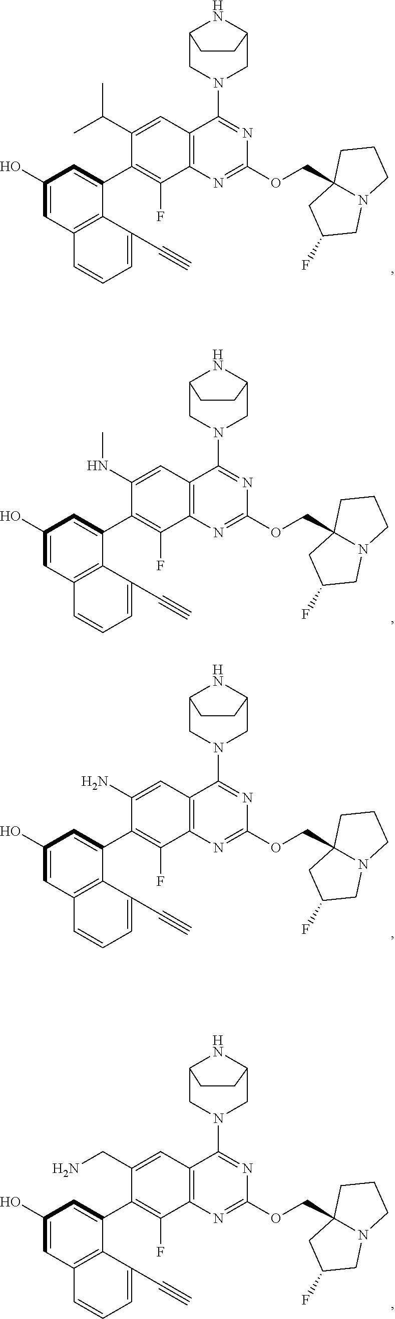

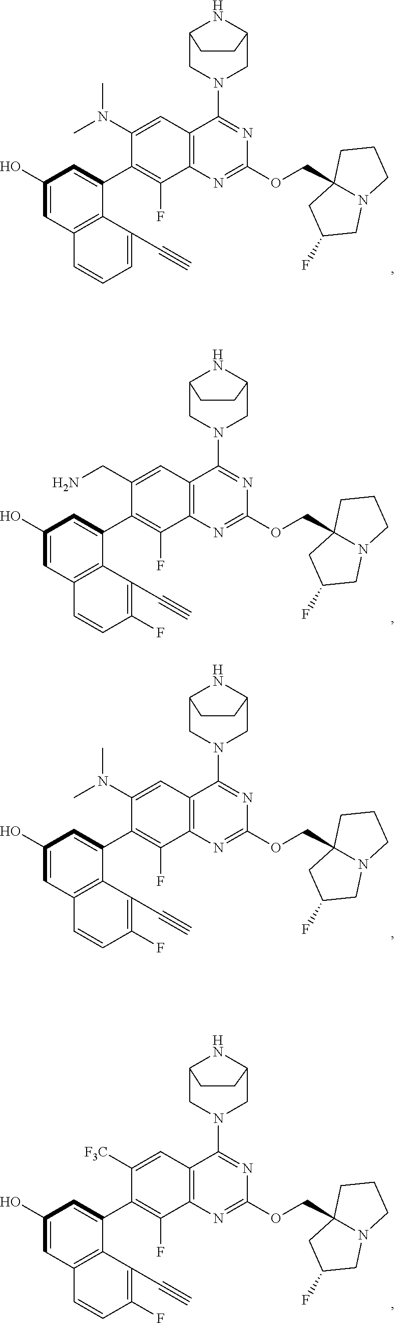

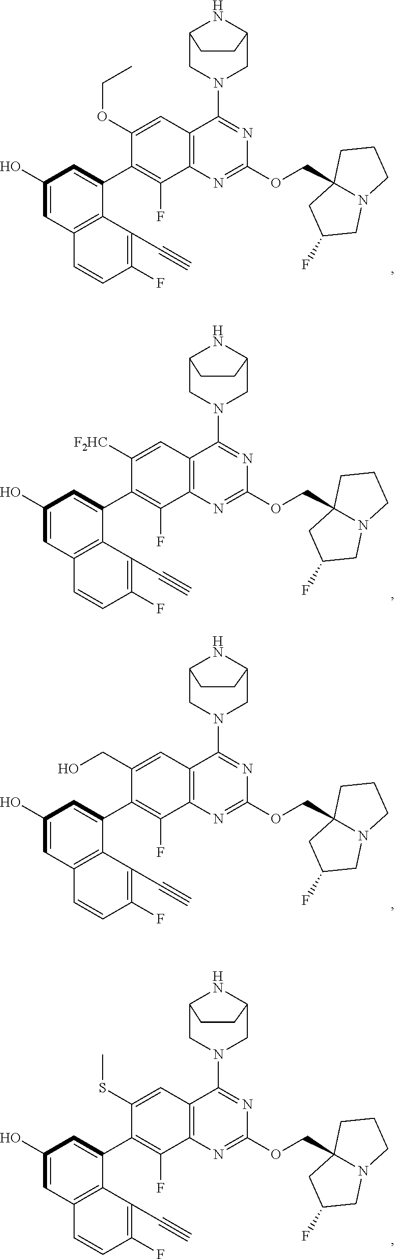

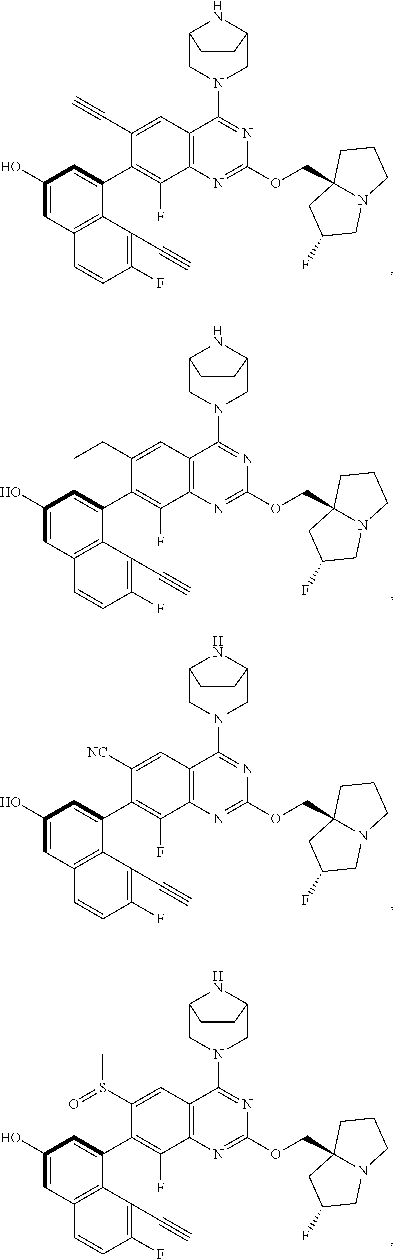

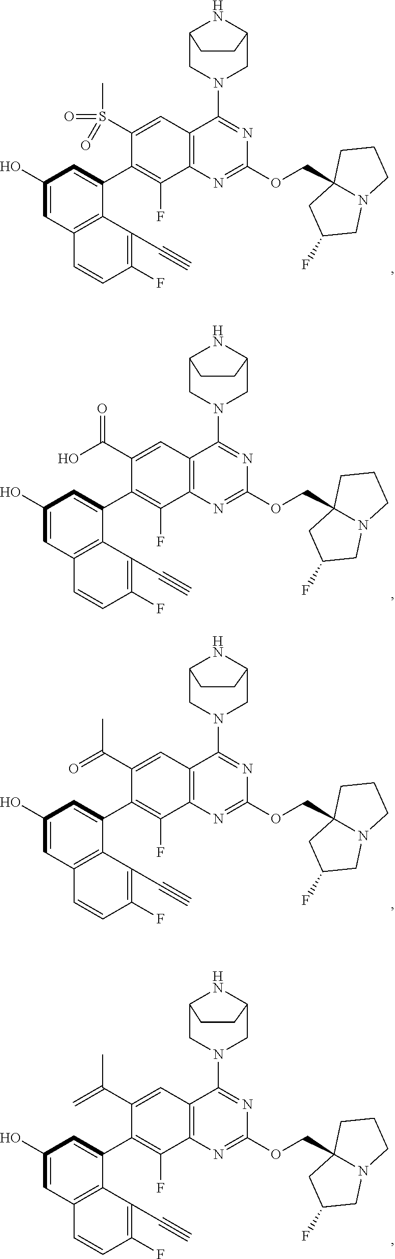

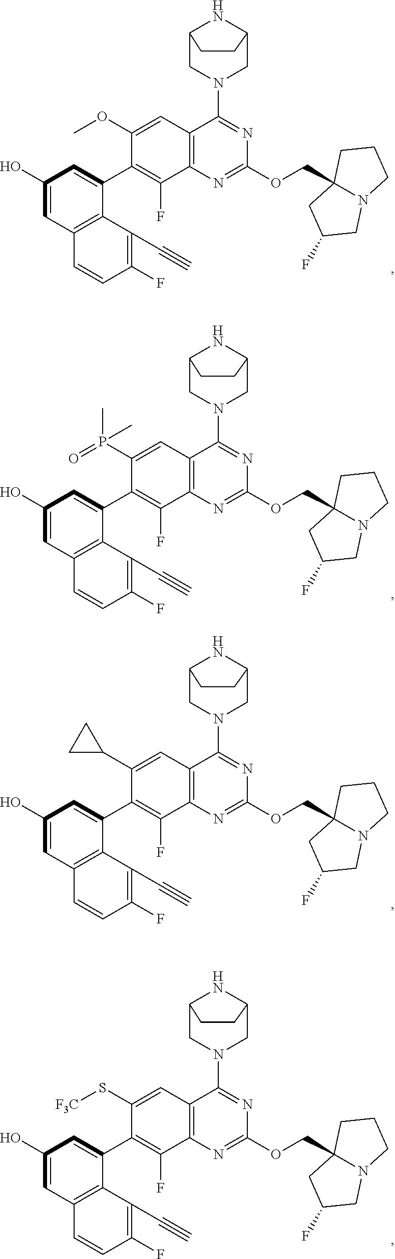

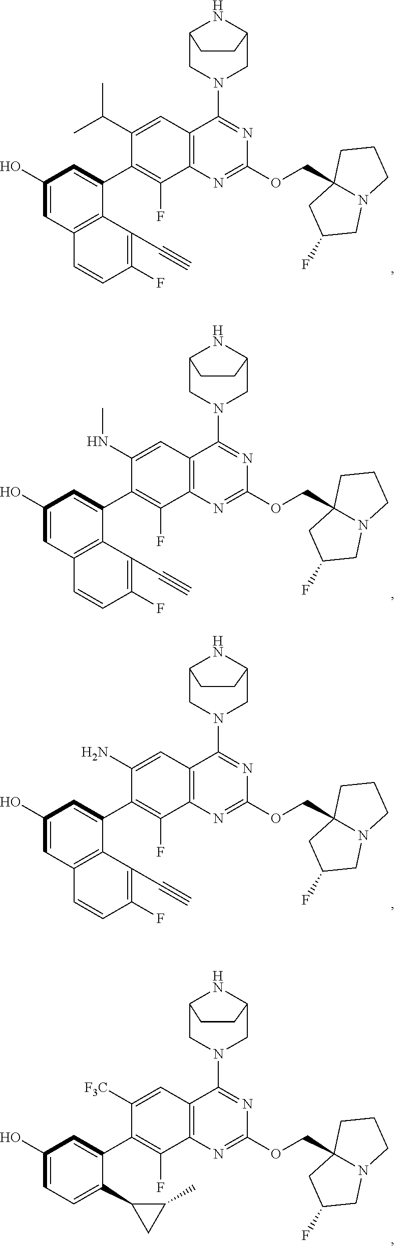

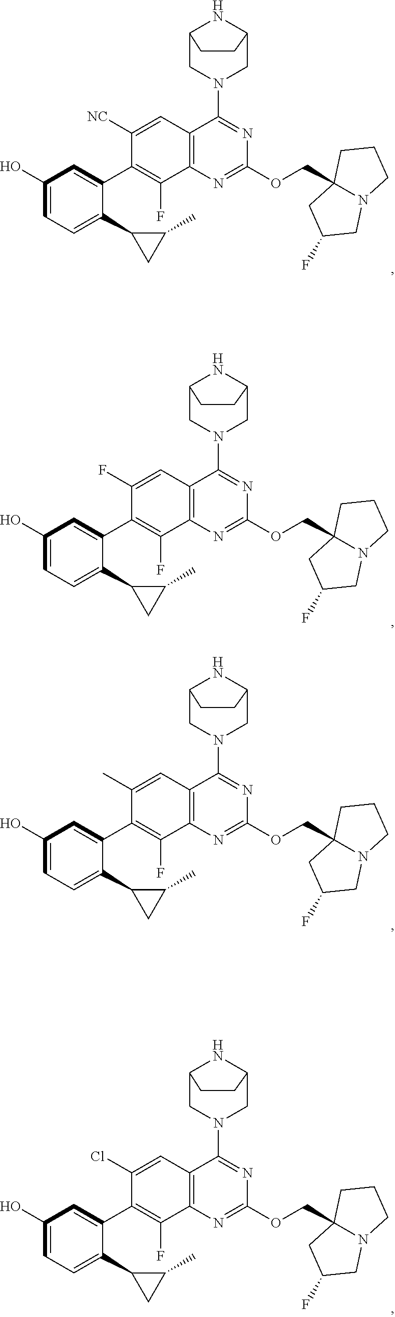

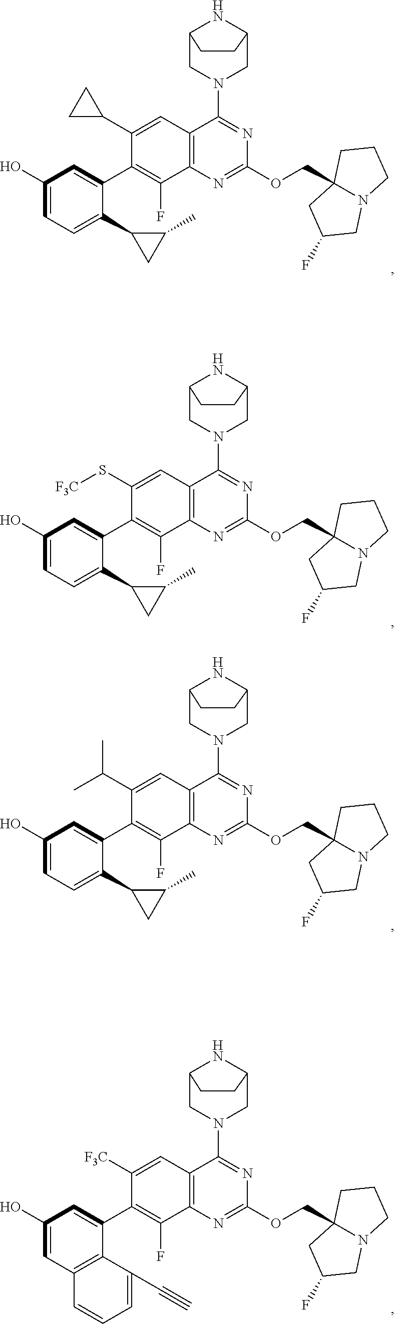

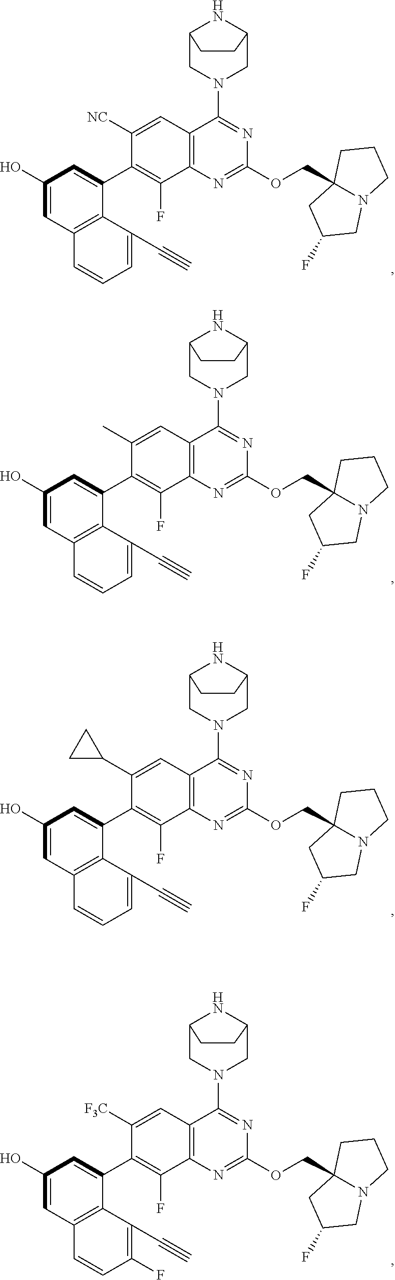

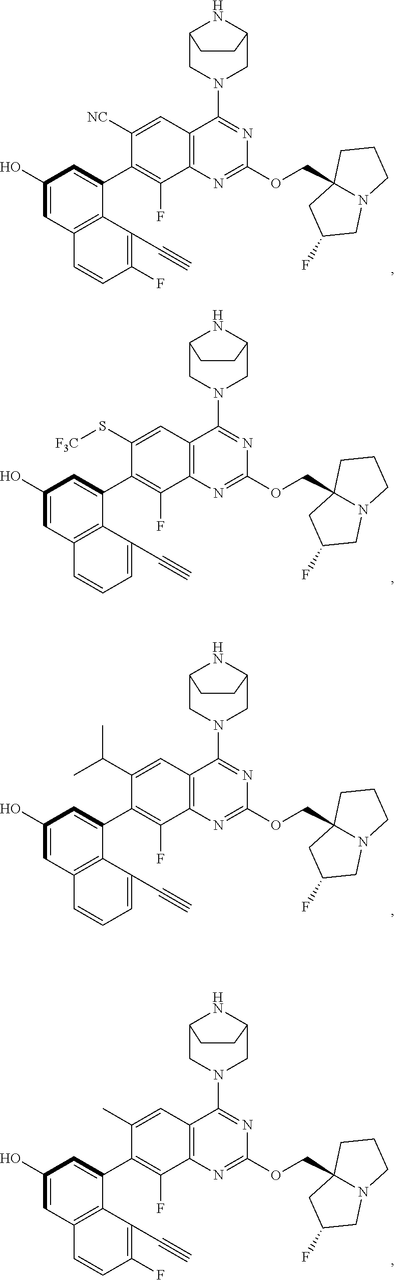

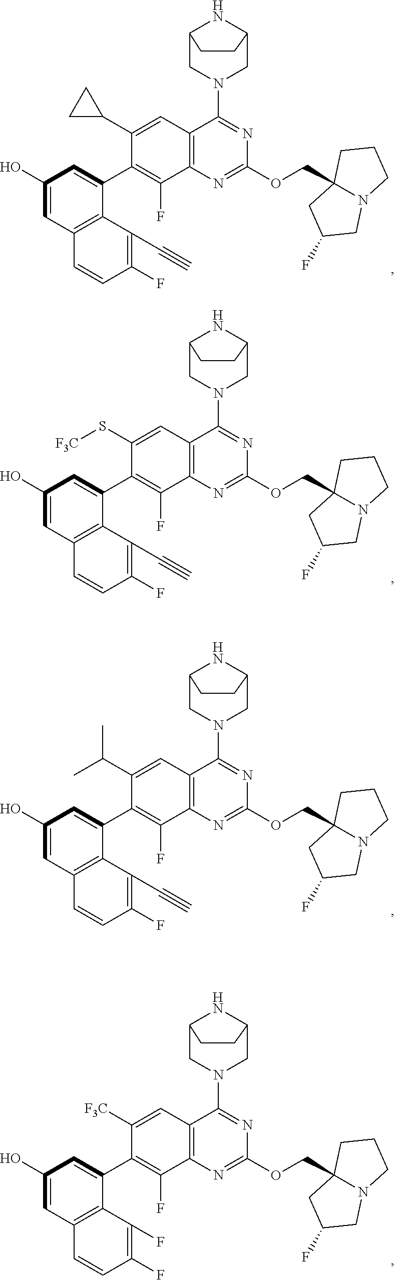

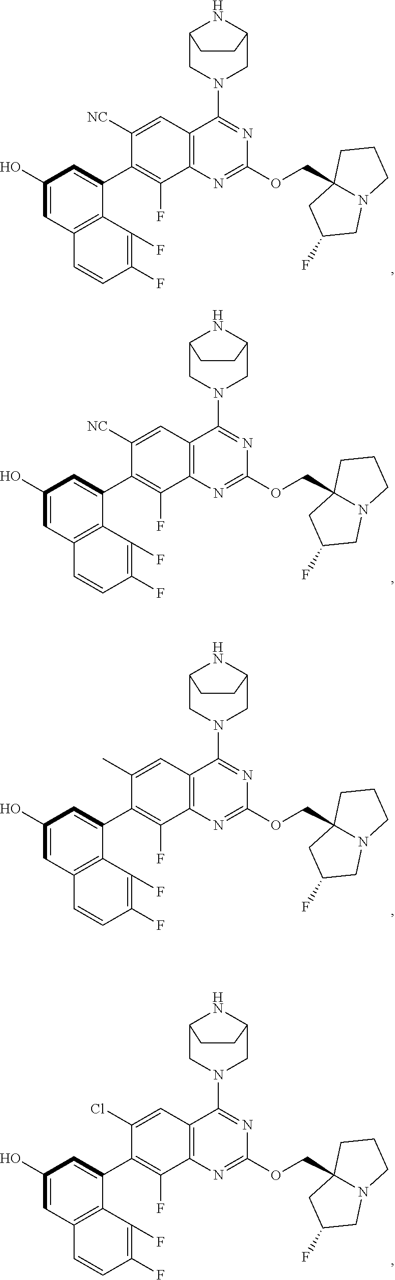

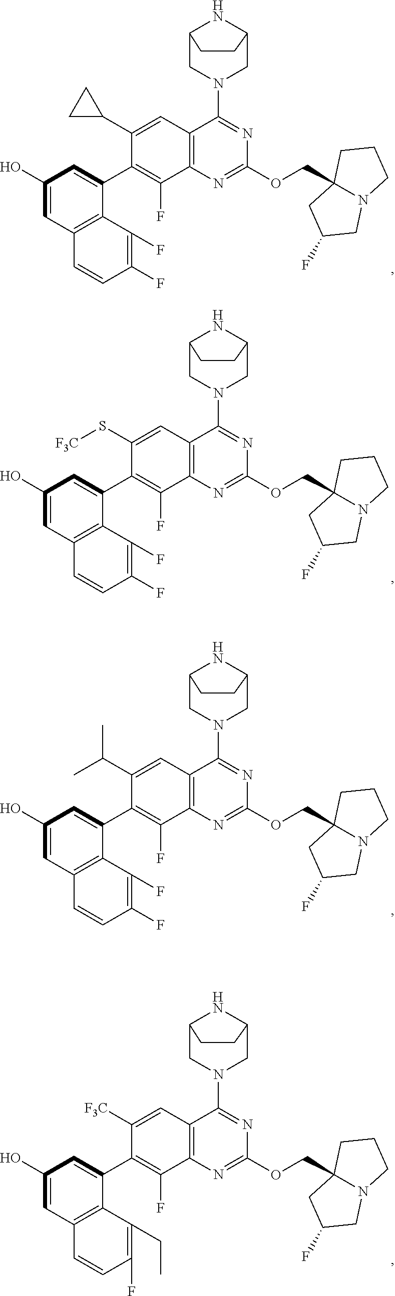

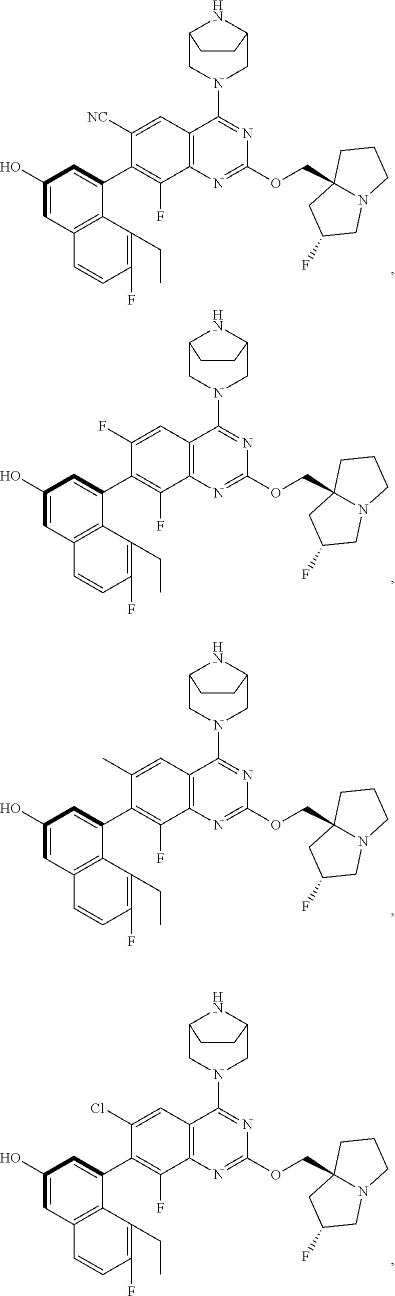

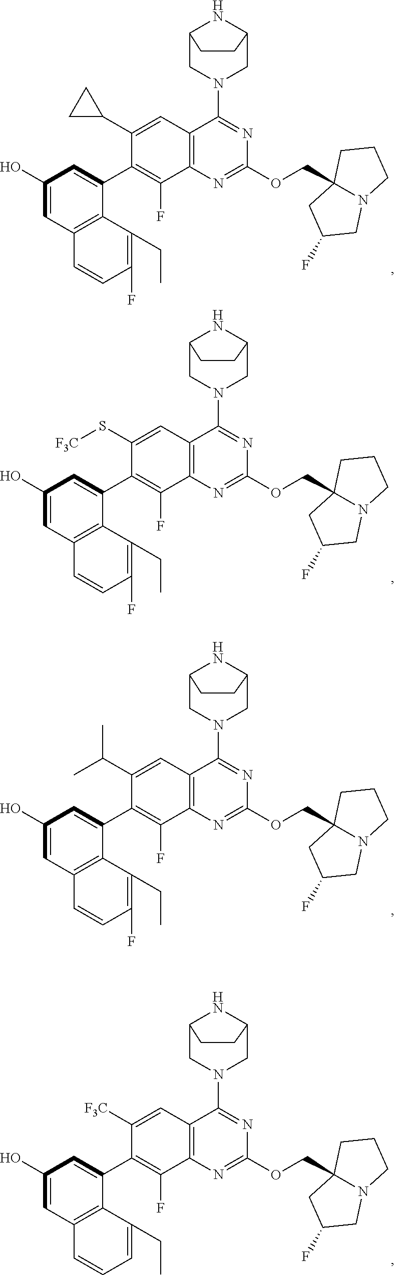

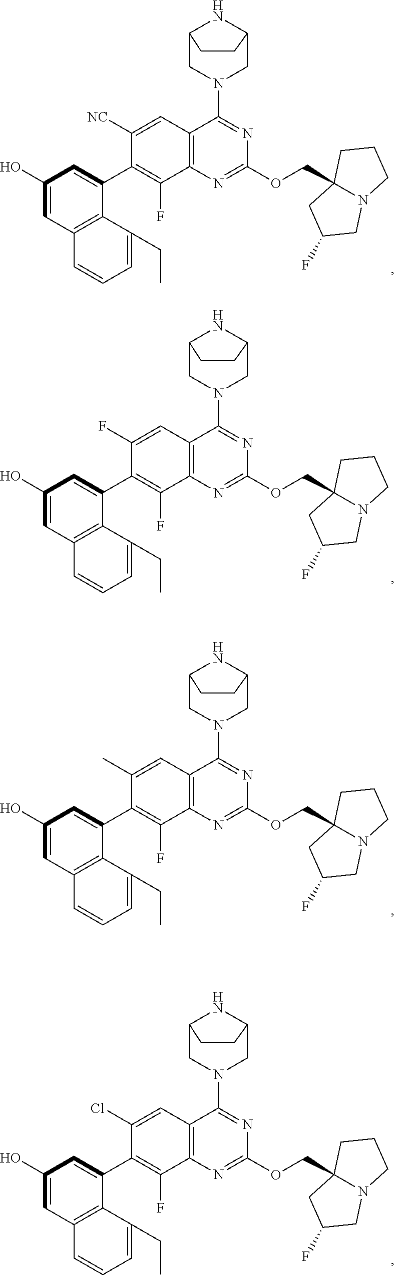

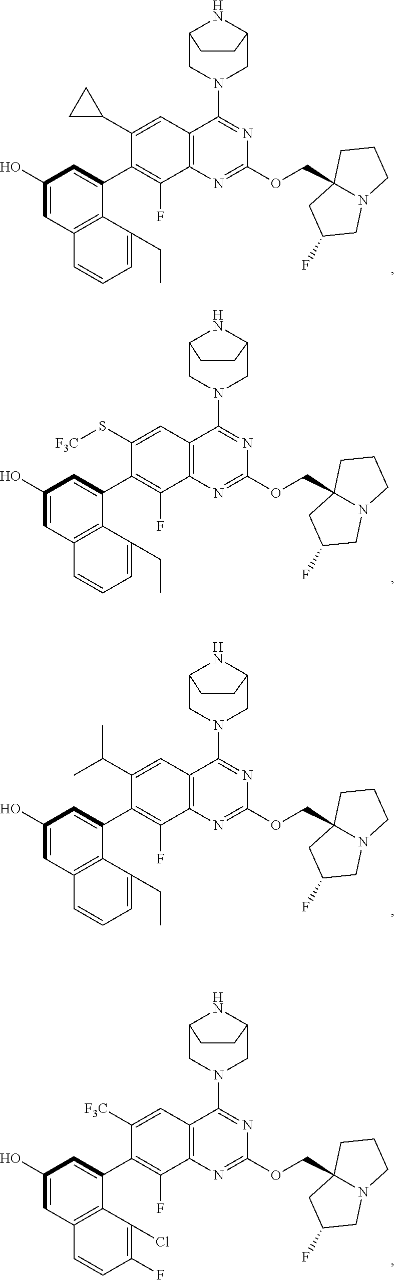

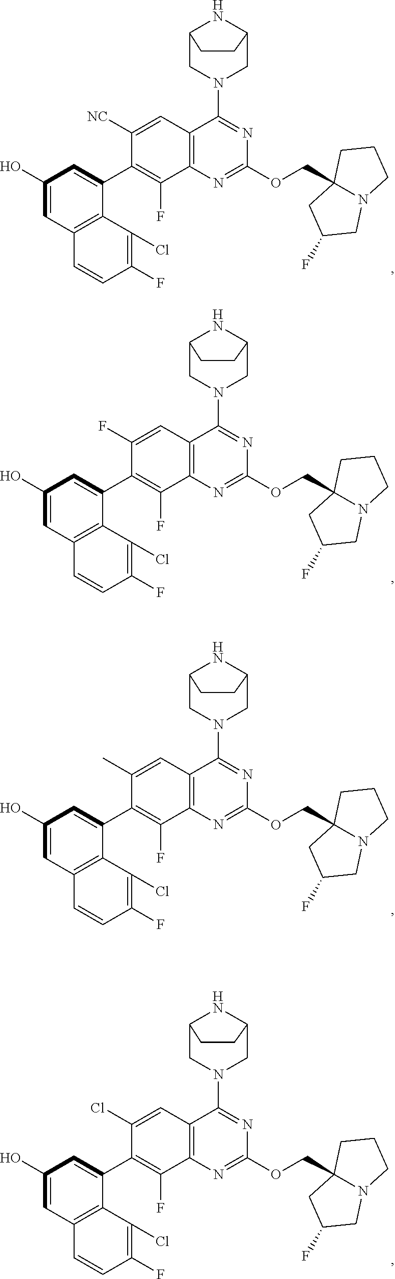

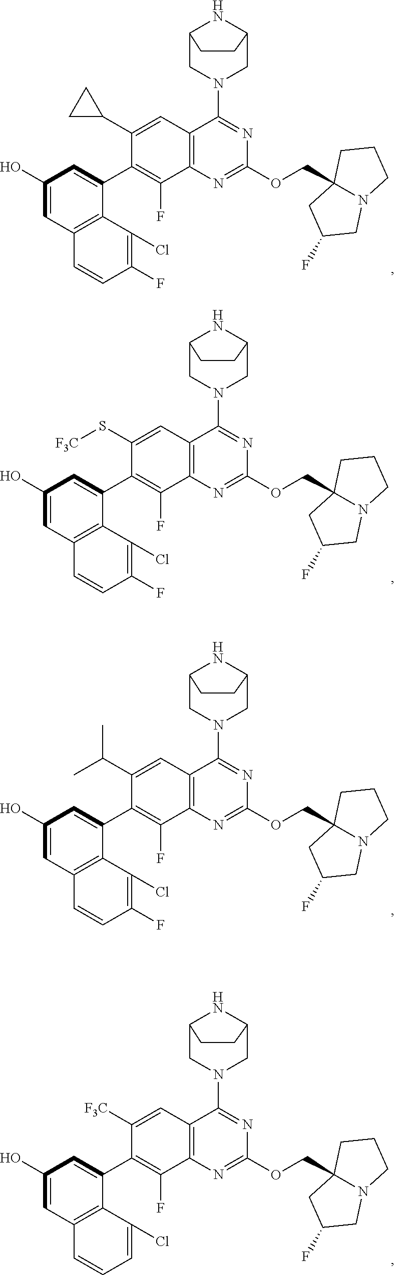

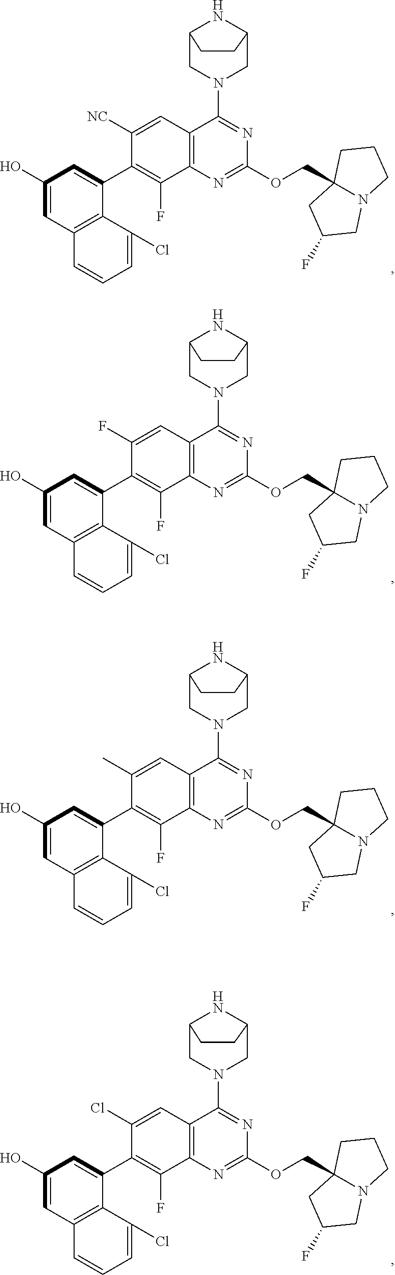

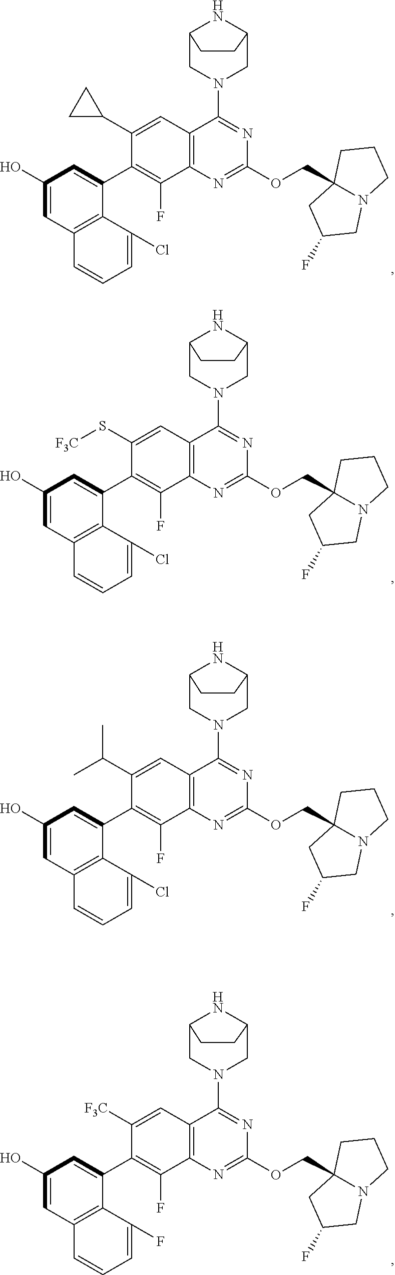

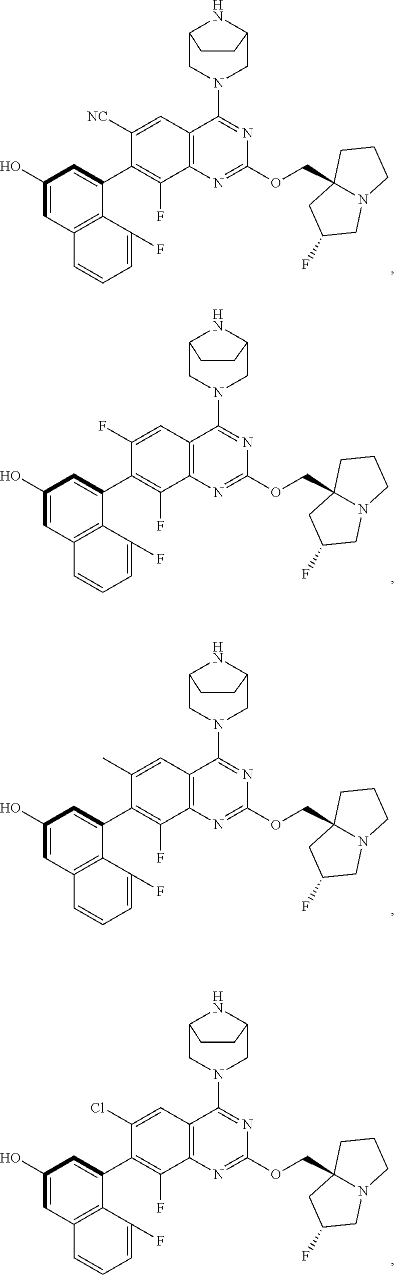

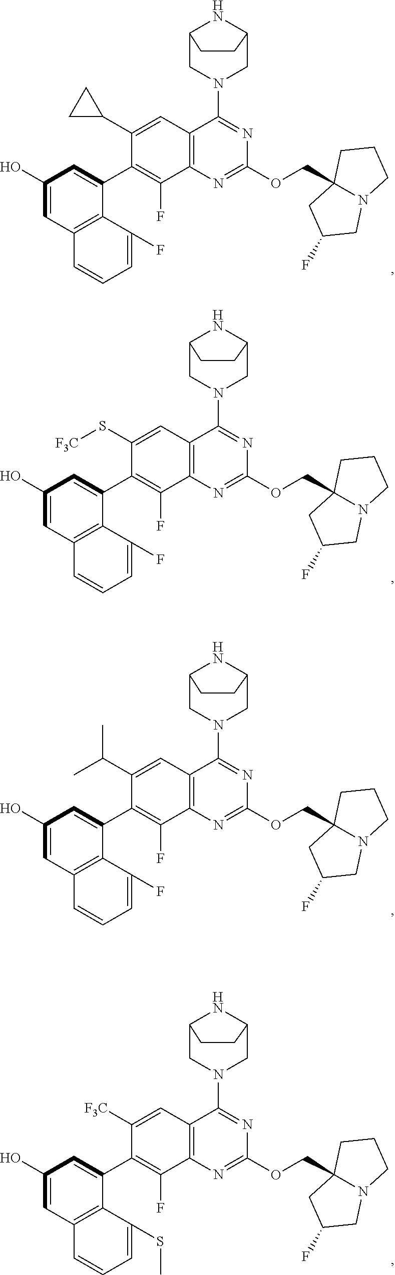

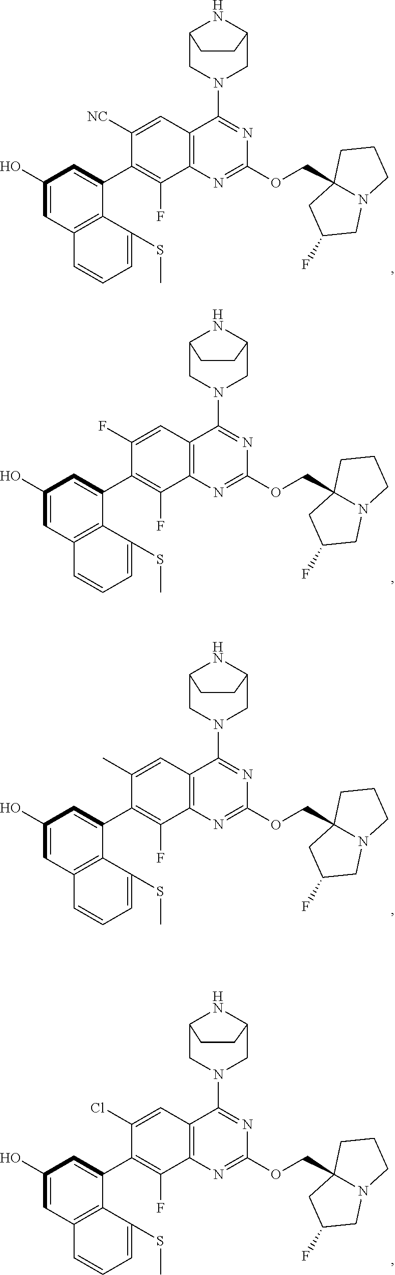

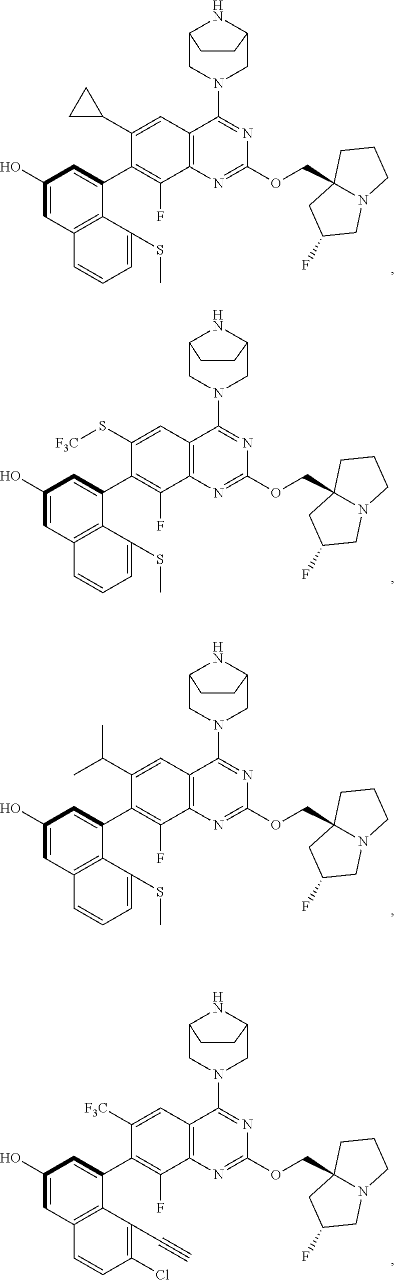

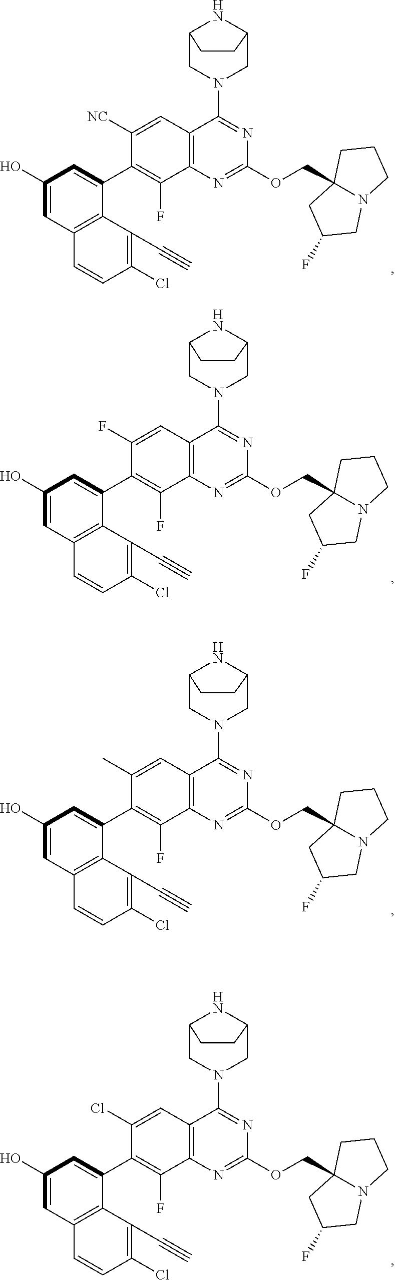

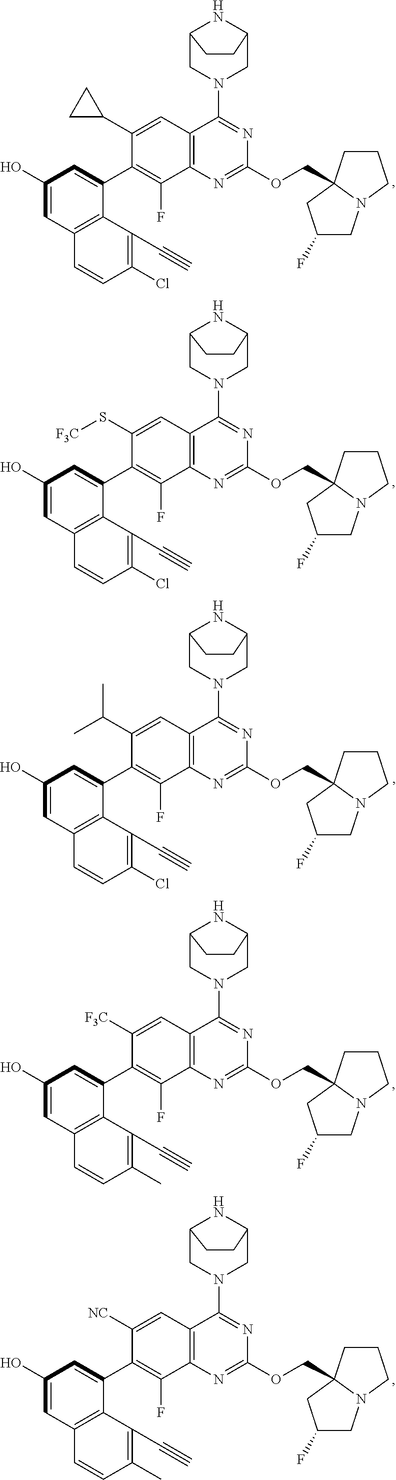

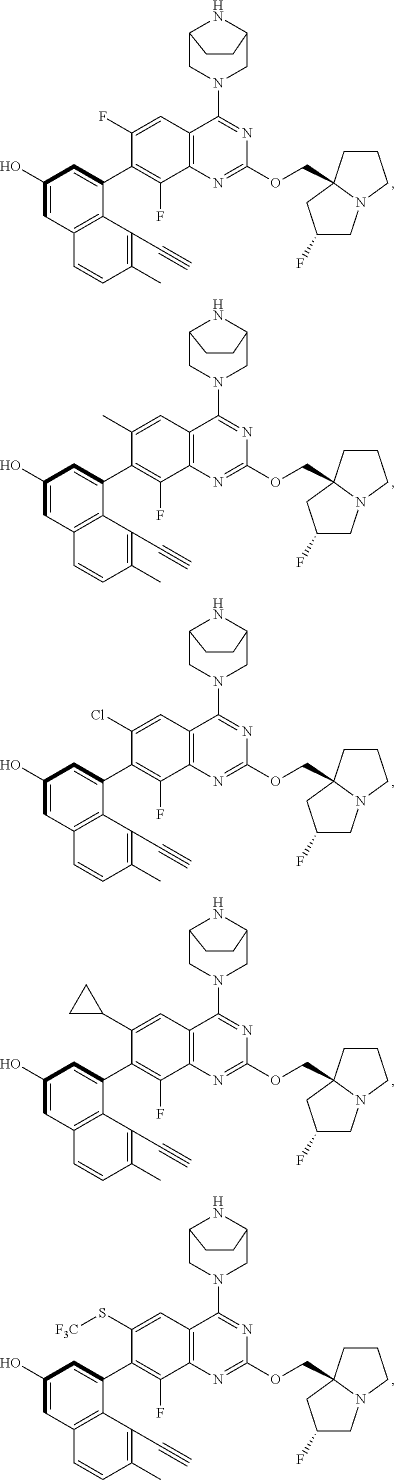

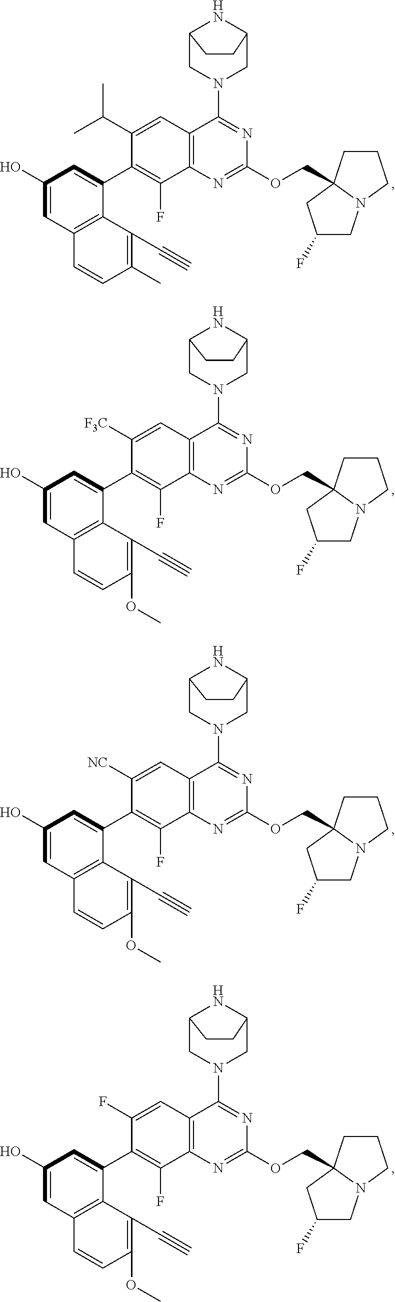

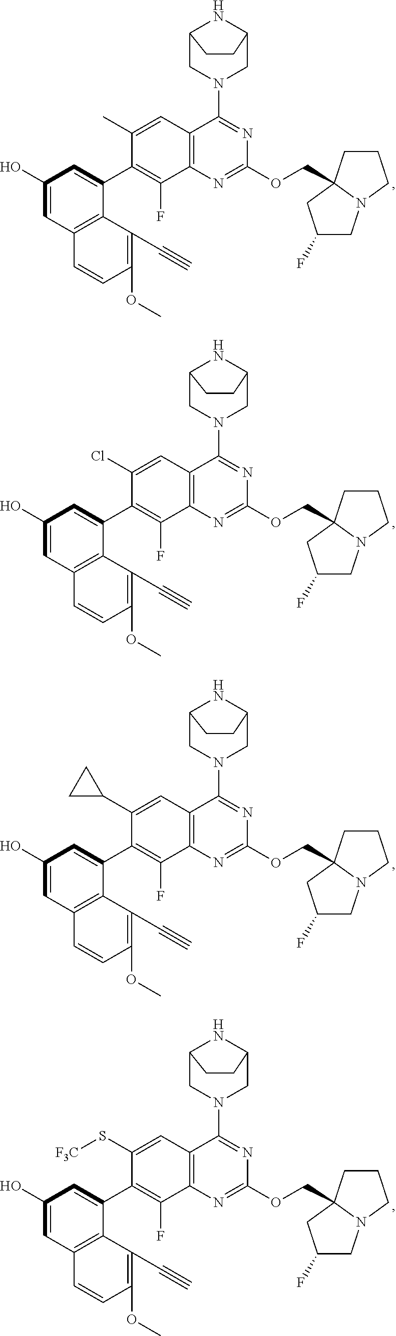

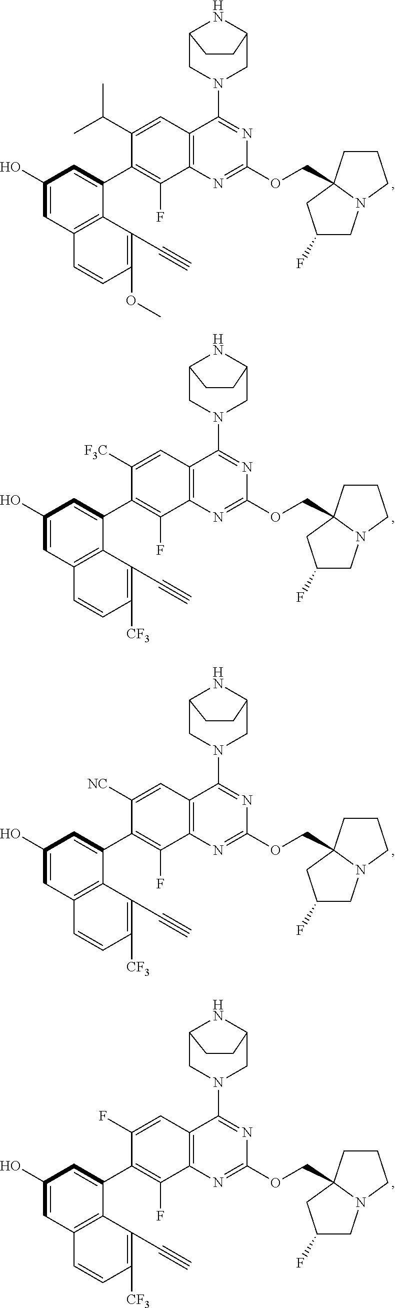

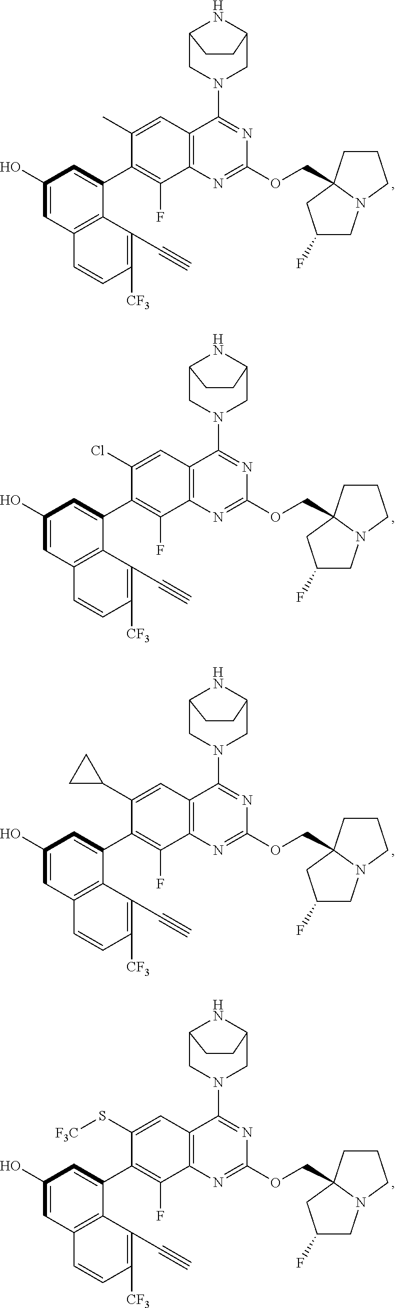

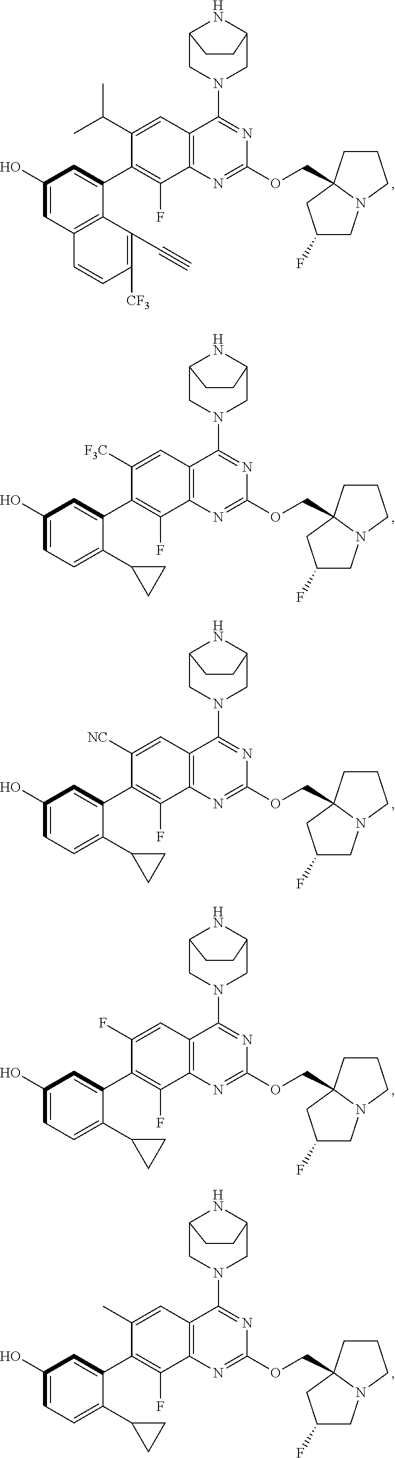

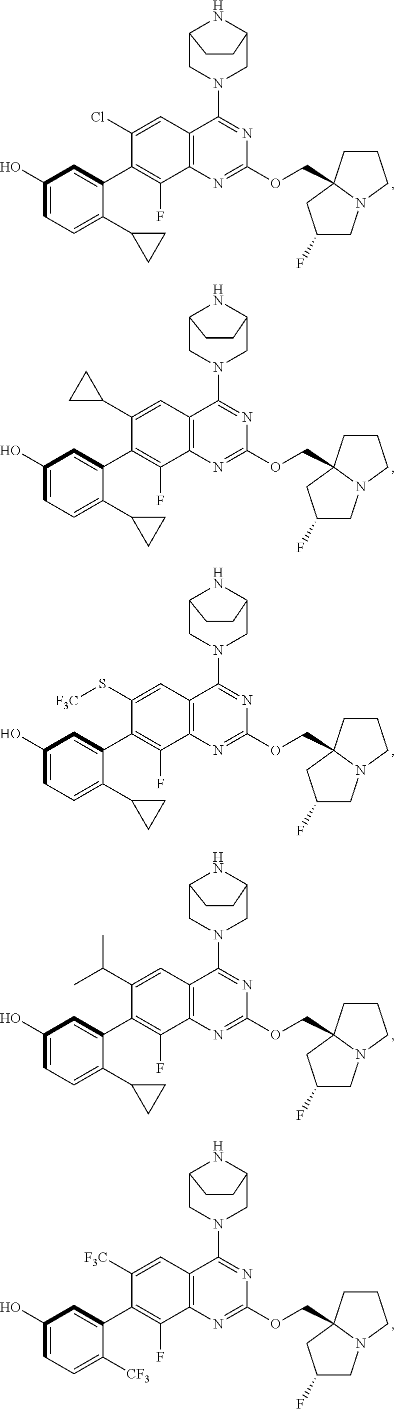

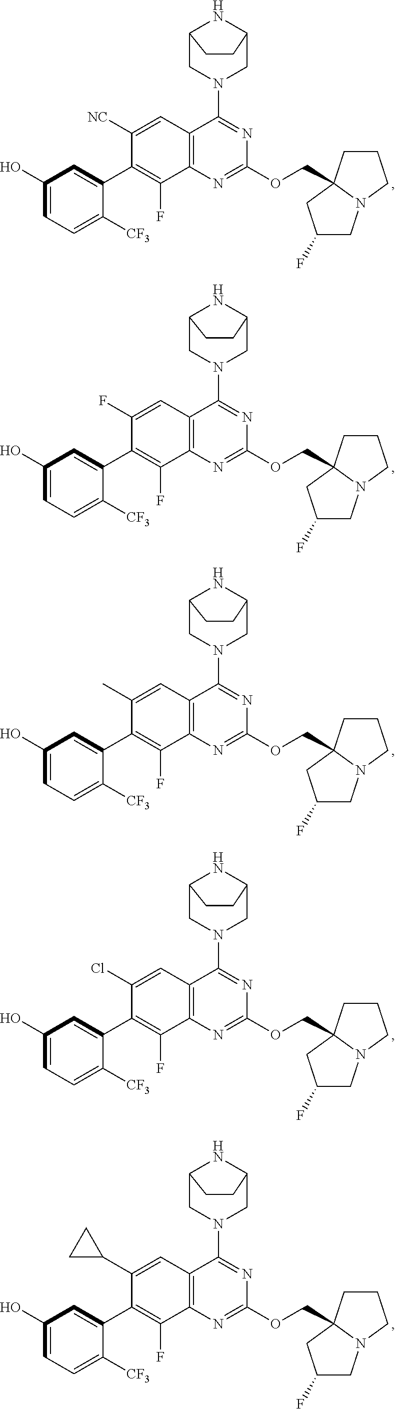

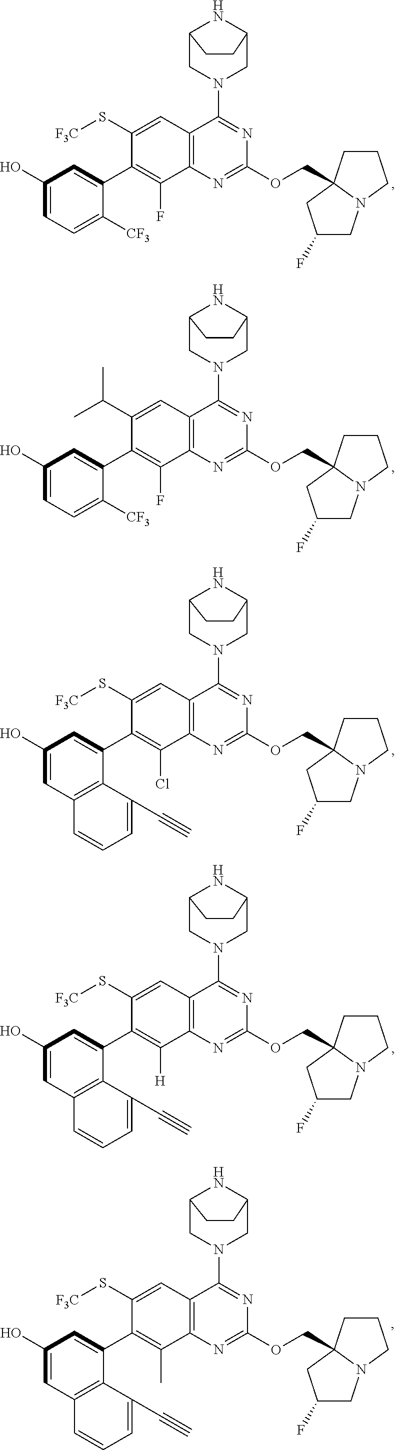

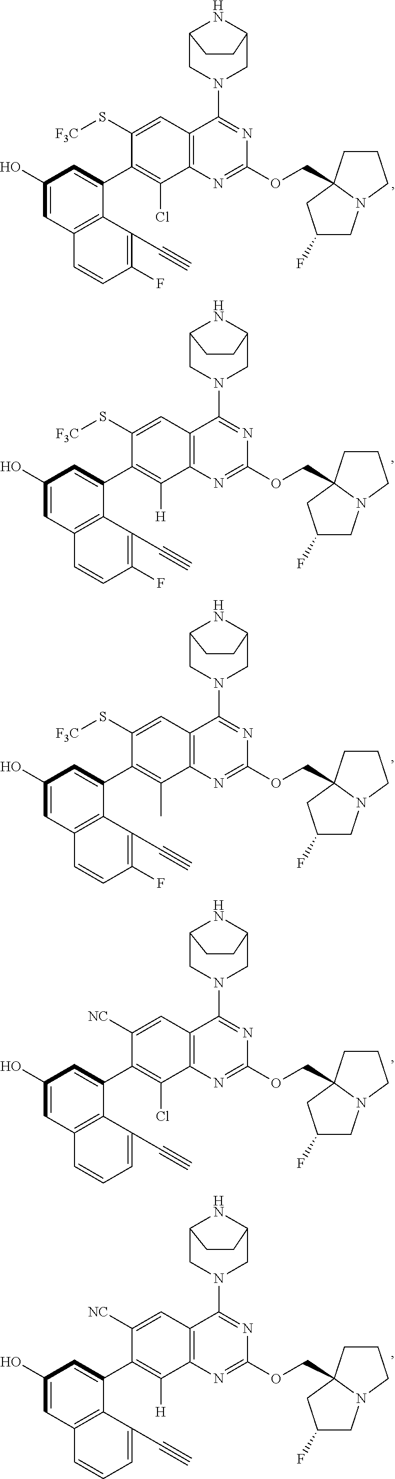

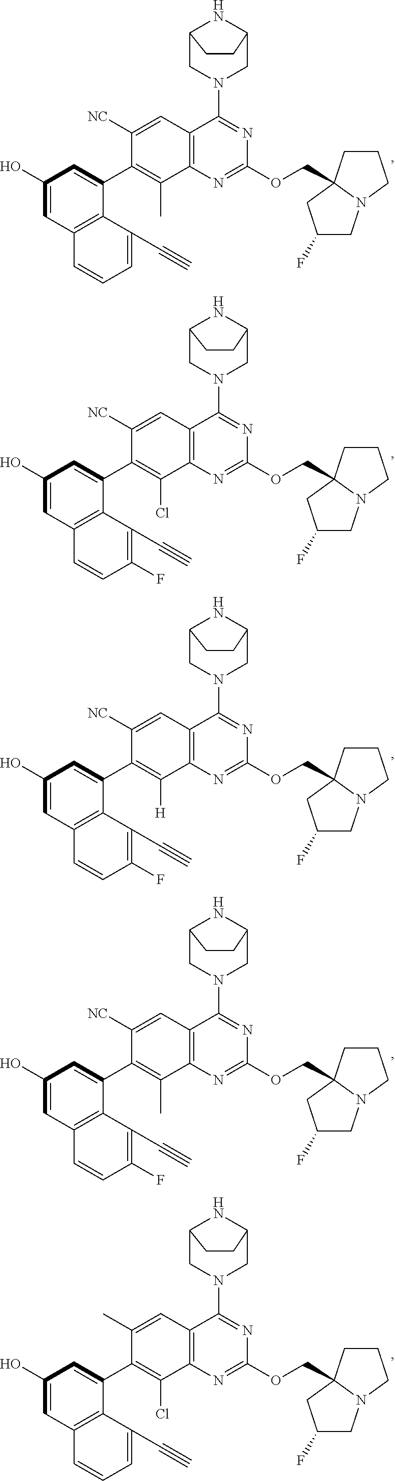

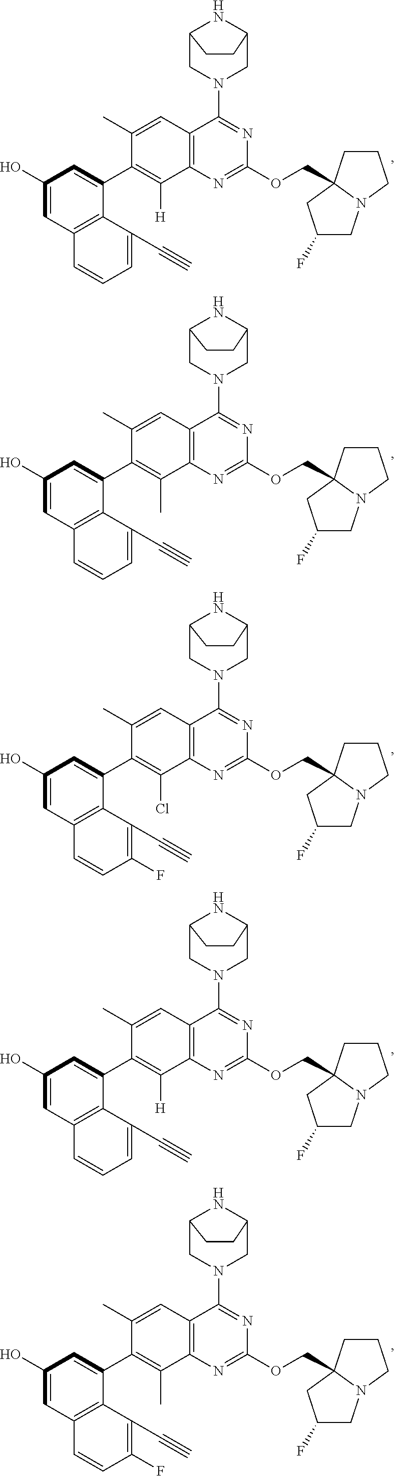

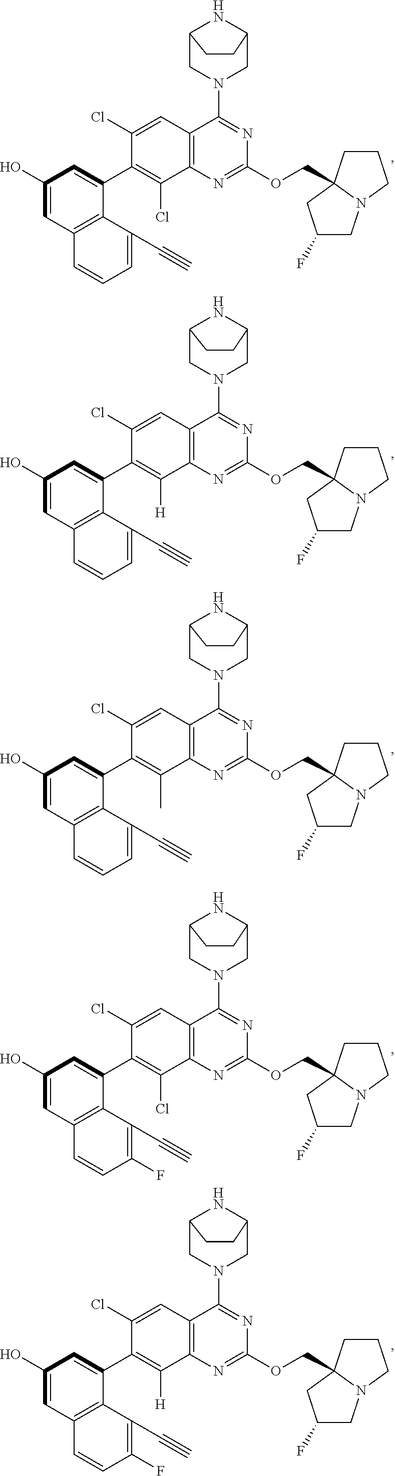

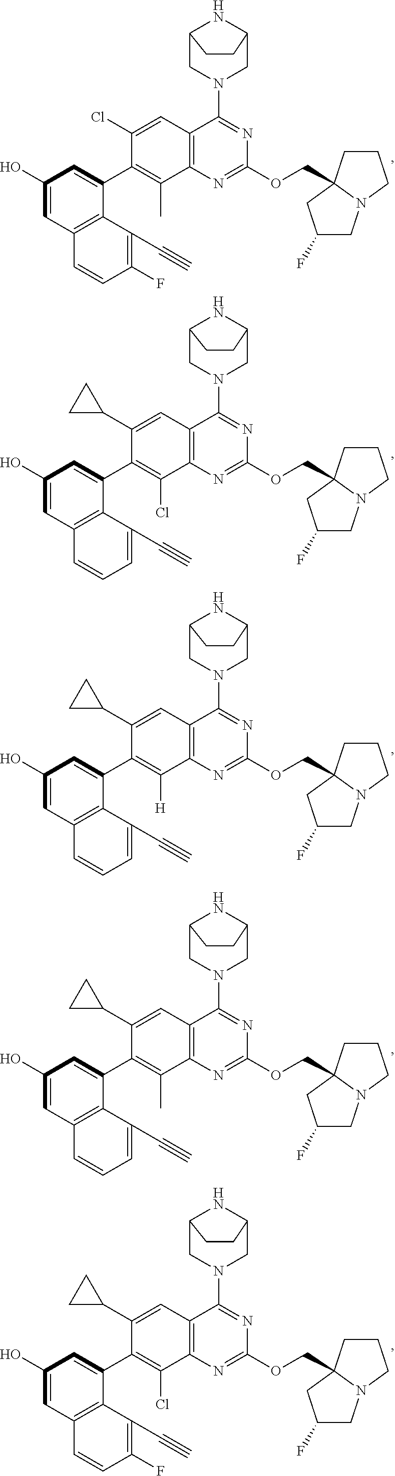

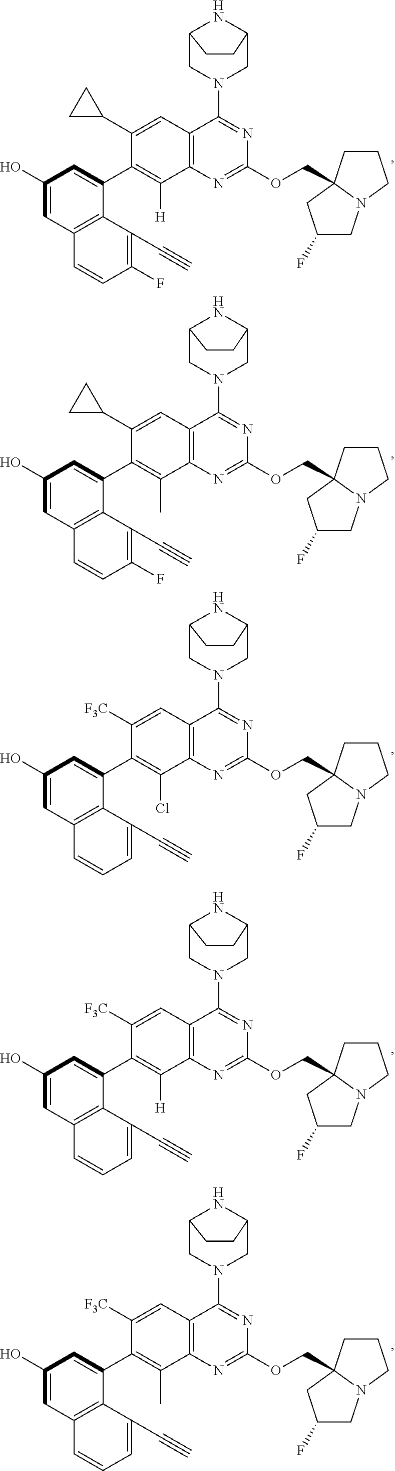

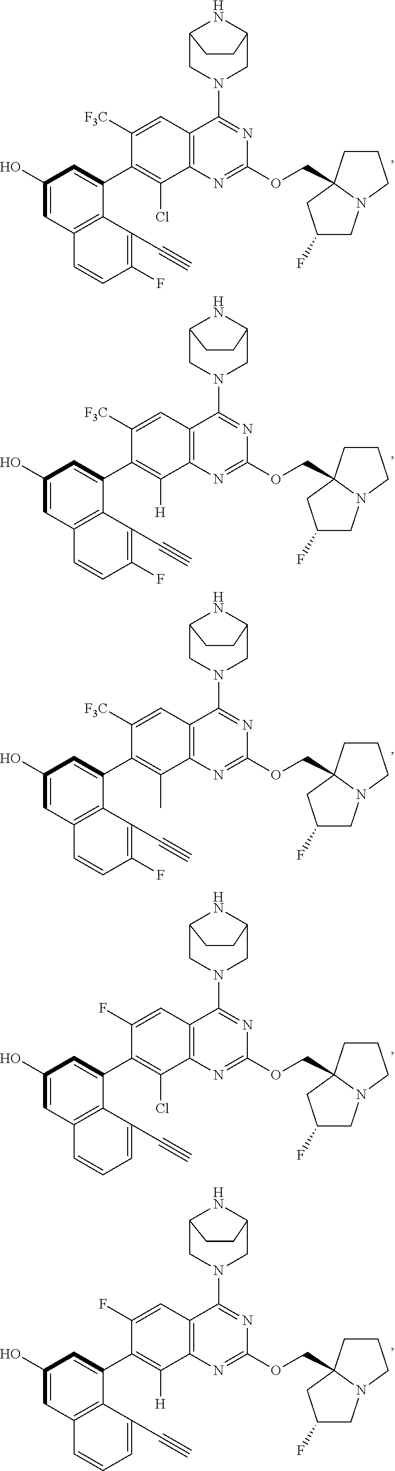

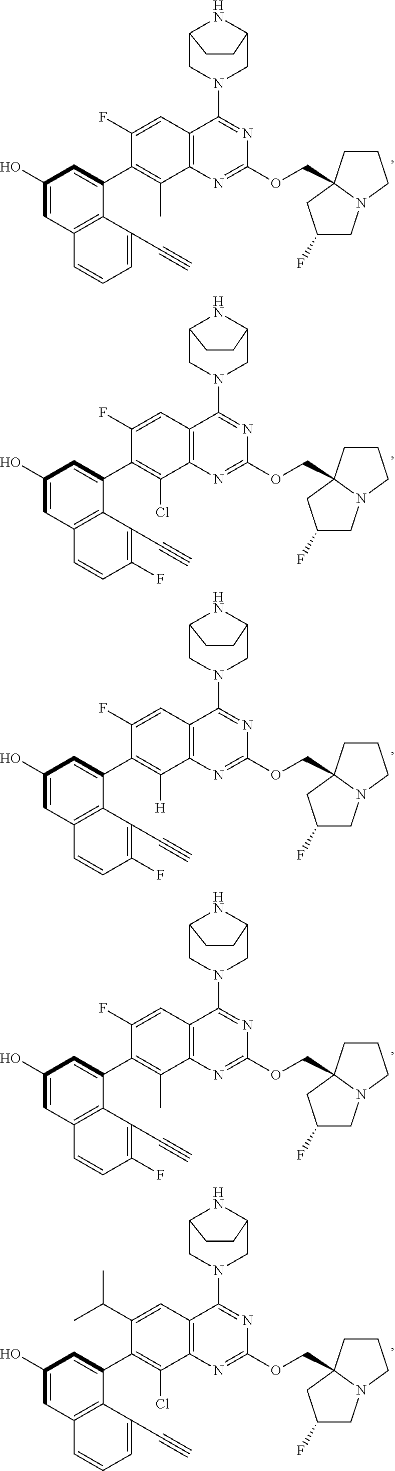

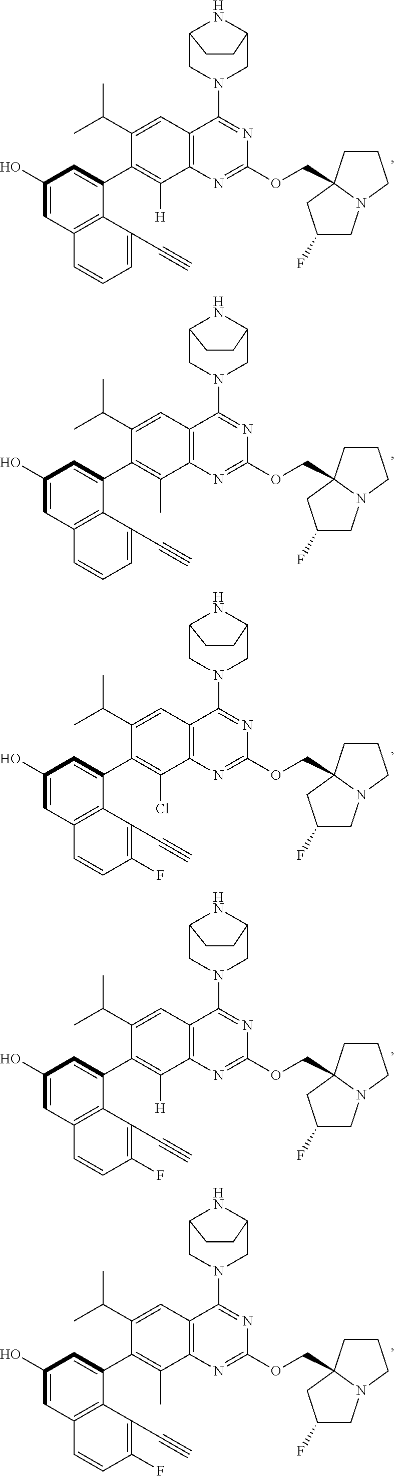

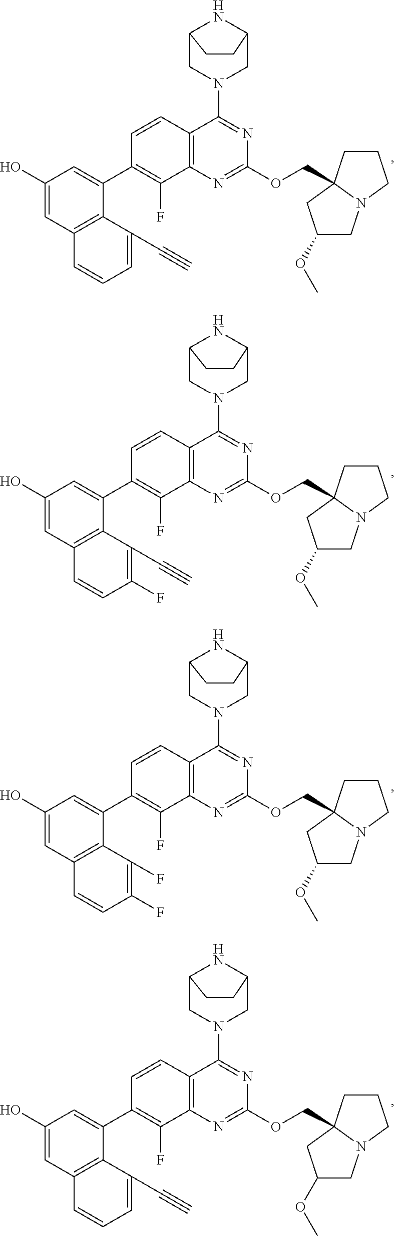 [SMILES: C#Cc1cccc2cc(O)cc(-c3ccc4c(N5CC6CCC(C5)N6)nc(OC[C@@]56CCCN5C[C@H](OC)C6)nc4c3F)c12 ; C#Cc1c(F)ccc2cc(O)cc(-c3ccc4c(N5CC6CCC(C5)N6)nc(OC[C@@]56CCCN5C[C@H](OC)C6)nc4c3F)c12 ; CO[C@H]1CN2CCC[C@@]2(COc2nc(N3CC4CCC(C3)N4)c3ccc(-c4cc(O)cc5ccc(F)c(F)c45)c(F)c3n2)C1 ; C#Cc1cccc2cc(O)cc(-c3ccc4c(N5CC6CCC(C5)N6)nc(OC[C@@]56CCCN5CC(OC)C6)nc4c3F)c12]

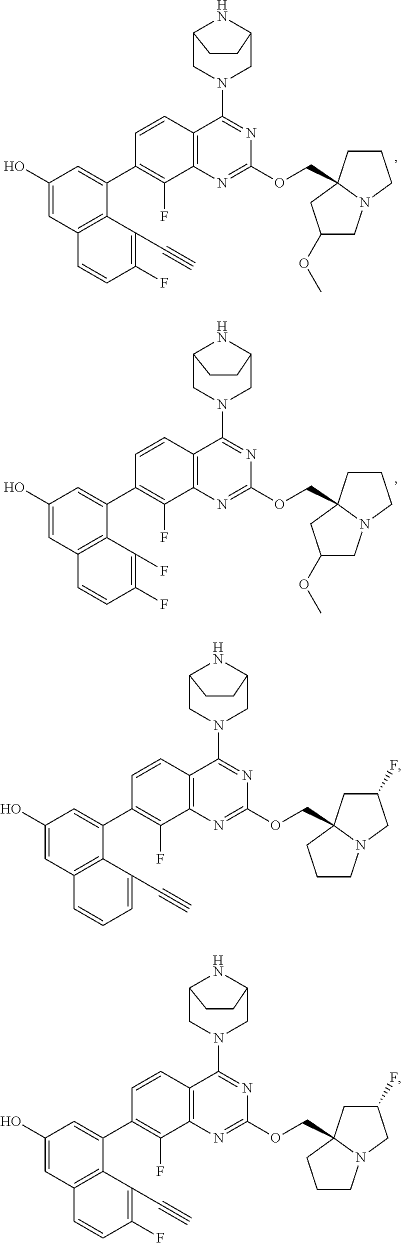 [SMILES: C#Cc1c(F)ccc2cc(O)cc(-c3ccc4c(N5CC6CCC(C5)N6)nc(OC[C@@]56CCCN5CC(OC)C6)nc4c3F)c12 ; COC1CN2CCC[C@@]2(COc2nc(N3CC4CCC(C3)N4)c3ccc(-c4cc(O)cc5ccc(F)c(F)c45)c(F)c3n2)C1 ; C#Cc1cccc2cc(O)cc(-c3ccc4c(N5CC6CCC(C5)N6)nc(OC[C@]56CCCN5C[C@@H](F)C6)nc4c3F)c12 ; C#Cc1c(F)ccc2cc(O)cc(-c3ccc4c(N5CC6CCC(C5)N6)nc(OC[C@]56CCCN5C[C@@H](F)C6)nc4c3F)c12]

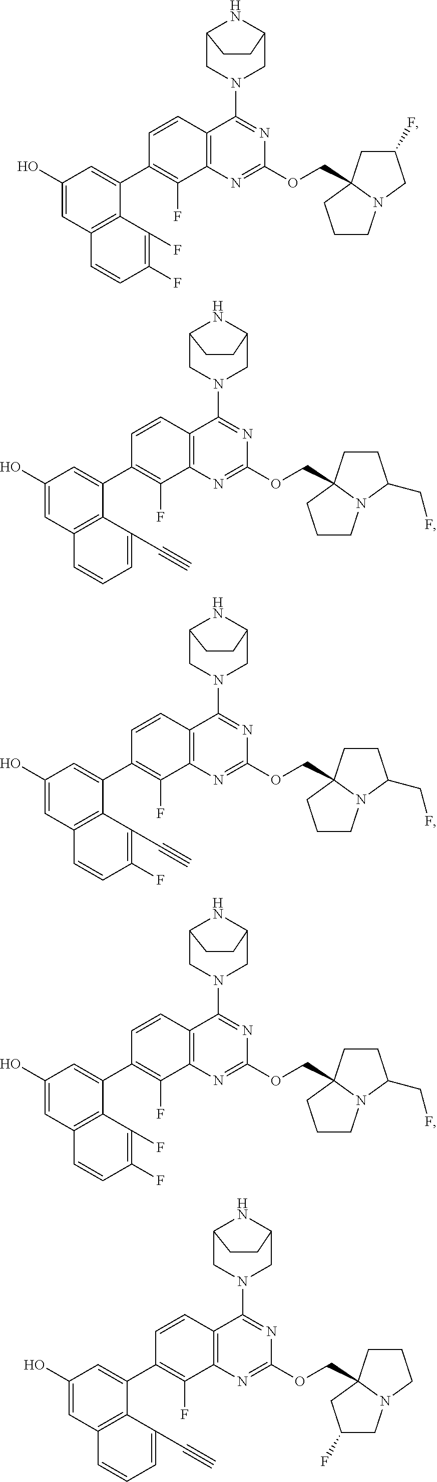 [SMILES: Oc1cc(-c2ccc3c(N4CC5CCC(C4)N5)nc(OC[C@]45CCCN4C[C@@H](F)C5)nc3c2F)c2c(F)c(F)ccc2c1 ; C#Cc1cccc2cc(O)cc(-c3ccc4c(N5CC6CCC(C5)N6)nc(OC[C@]56CCCN5C(CF)CC6)nc4c3F)c12 ; C#Cc1c(F)ccc2cc(O)cc(-c3ccc4c(N5CC6CCC(C5)N6)nc(OC[C@]56CCCN5C(CF)CC6)nc4c3F)c12 ; Oc1cc(-c2ccc3c(N4CC5CCC(C4)N5)nc(OC[C@]45CCCN4C(CF)CC5)nc3c2F)c2c(F)c(F)ccc2c1 ; C#Cc1cccc2cc(O)cc(-c3ccc4c(N5CC6CCC(C5)N6)nc(OC[C@@]56CCCN5C[C@H](F)C6)nc4c3F)c12]

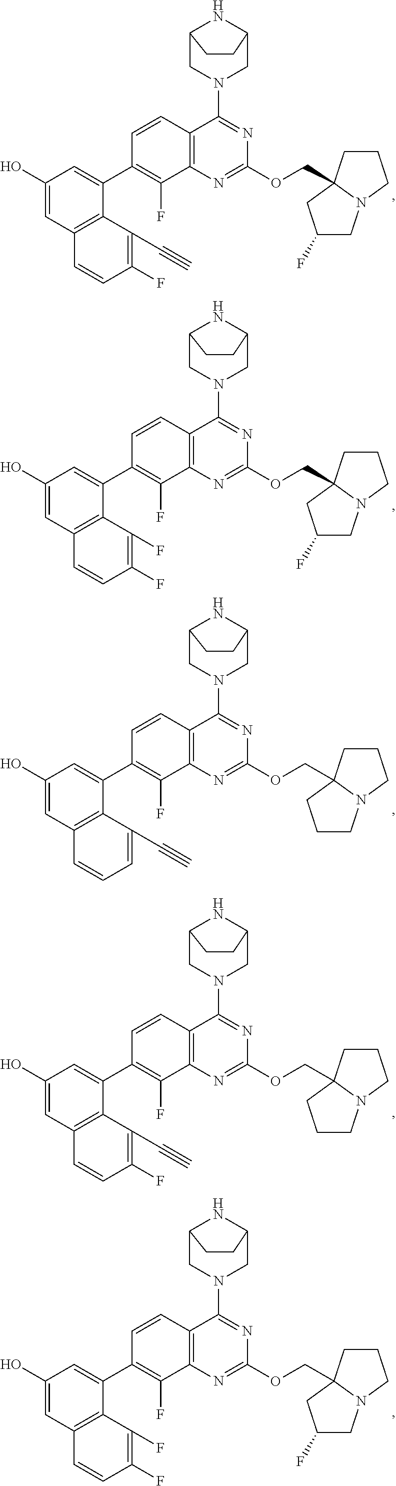 [SMILES: C#Cc1c(F)ccc2cc(O)cc(-c3ccc4c(N5CC6CCC(C5)N6)nc(OC[C@@]56CCCN5C[C@H](F)C6)nc4c3F)c12 ; Oc1cc(-c2ccc3c(N4CC5CCC(C4)N5)nc(OC[C@@]45CCCN4C[C@H](F)C5)nc3c2F)c2c(F)c(F)ccc2c1 ; C#Cc1cccc2cc(O)cc(-c3ccc4c(N5CC6CCC(C5)N6)nc(OCC56CCCN5CCC6)nc4c3F)c12 ; C#Cc1c(F)ccc2cc(O)cc(-c3ccc4c(N5CC6CCC(C5)N6)nc(OCC56CCCN5CCC6)nc4c3F)c12 ; Oc1cc(-c2ccc3c(N4CC5CCC(C4)N5)nc(OCC45CCCN4C[C@H](F)C5)nc3c2F)c2c(F)c(F)ccc2c1]

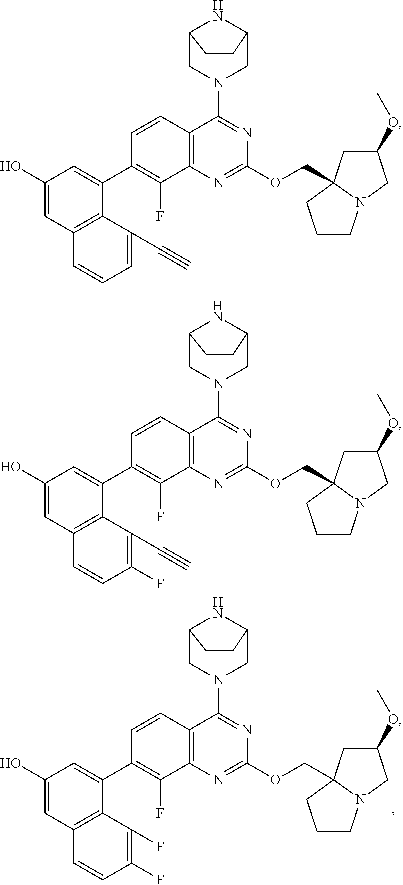 [SMILES: C#Cc1cccc2cc(O)cc(-c3ccc4c(N5CC6CCC(C5)N6)nc(OC[C@]56CCCN5C[C@H](OC)C6)nc4c3F)c12 ; C#Cc1c(F)ccc2cc(O)cc(-c3ccc4c(N5CC6CCC(C5)N6)nc(OC[C@]56CCCN5C[C@H](OC)C6)nc4c3F)c12 ; CO[C@H]1CN2CCCC2(COc2nc(N3CC4CCC(C3)N4)c3ccc(-c4cc(O)cc5ccc(F)c(F)c45)c(F)c3n2)C1]

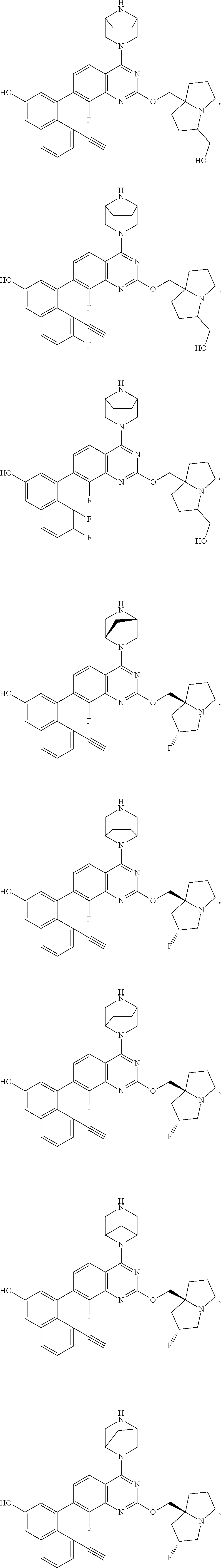

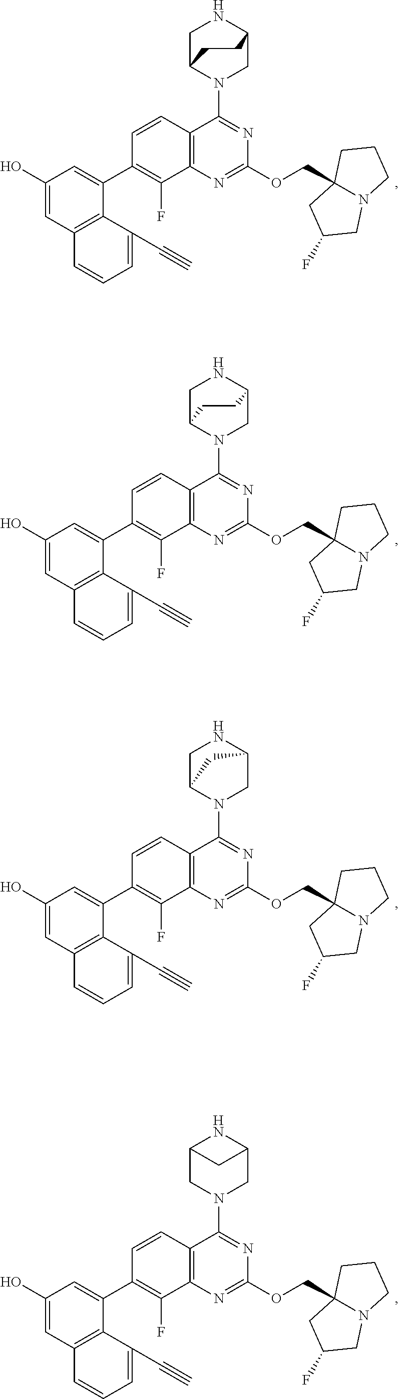 [SMILES: C#Cc1cccc2cc(O)cc(-c3ccc4c(N5C[C@@H]6CC[C@H]5CN6)nc(OC[C@@]56CCCN5C[C@H](F)C6)nc4c3F)c12 ; C#Cc1cccc2cc(O)cc(-c3ccc4c(N5C[C@H]6CC[C@@H]5CN6)nc(OC[C@@]56CCCN5C[C@H](F)C6)nc4c3F)c12 ; C#Cc1cccc2cc(O)cc(-c3ccc4c(N5C[C@H]6C[C@@H]5CN6)nc(OC[C@@]56CCCN5C[C@H](F)C6)nc4c3F)c12 ; C#Cc1cccc2cc(O)cc(-c3ccc4c(N5CC6CC(C5)N6)nc(OC[C@@]56CCCN5C[C@H](F)C6)nc4c3F)c12]

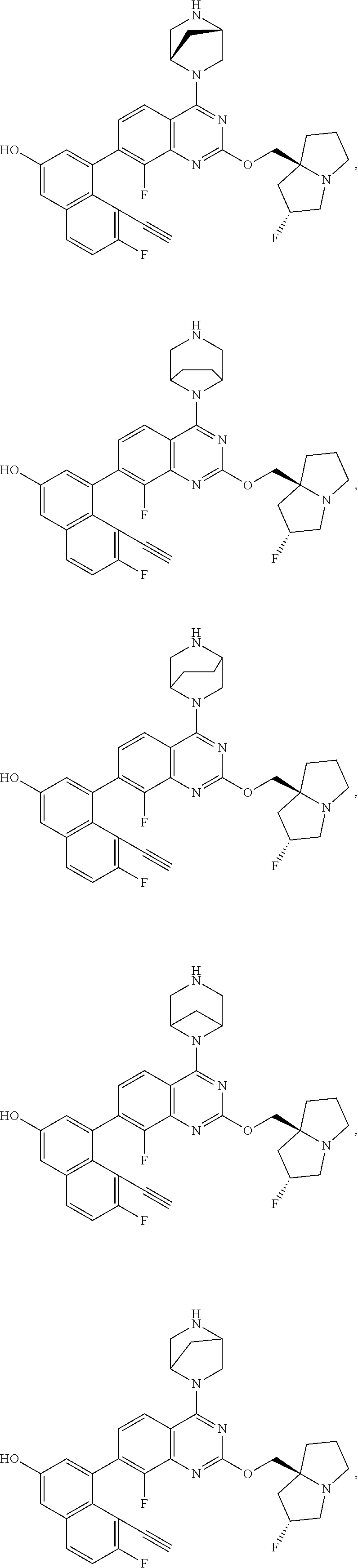 [SMILES: C#Cc1c(F)ccc2cc(O)cc(-c3ccc4c(N5C[C@@H]6C[C@H]5CN6)nc(OC[C@@]56CCCN5C[C@H](F)C6)nc4c3F)c12 ; C#Cc1c(F)ccc2cc(O)cc(-c3ccc4c(N5C6CCC5CNC6)nc(OC[C@@]56CCCN5C[C@H](F)C6)nc4c3F)c12 ; C#Cc1c(F)ccc2cc(O)cc(-c3ccc4c(N5CC6CCC5CN6)nc(OC[C@@]56CCCN5C[C@H](F)C6)nc4c3F)c12 ; C#Cc1c(F)ccc2cc(O)cc(-c3ccc4c(N5C6CNCC5C6)nc(OC[C@@]56CCCN5C[C@H](F)C6)nc4c3F)c12 ; C#Cc1c(F)ccc2cc(O)cc(-c3ccc4c(N5CC6CC5CN6)nc(OC[C@@]56CCCN5C[C@H](F)C6)nc4c3F)c12]

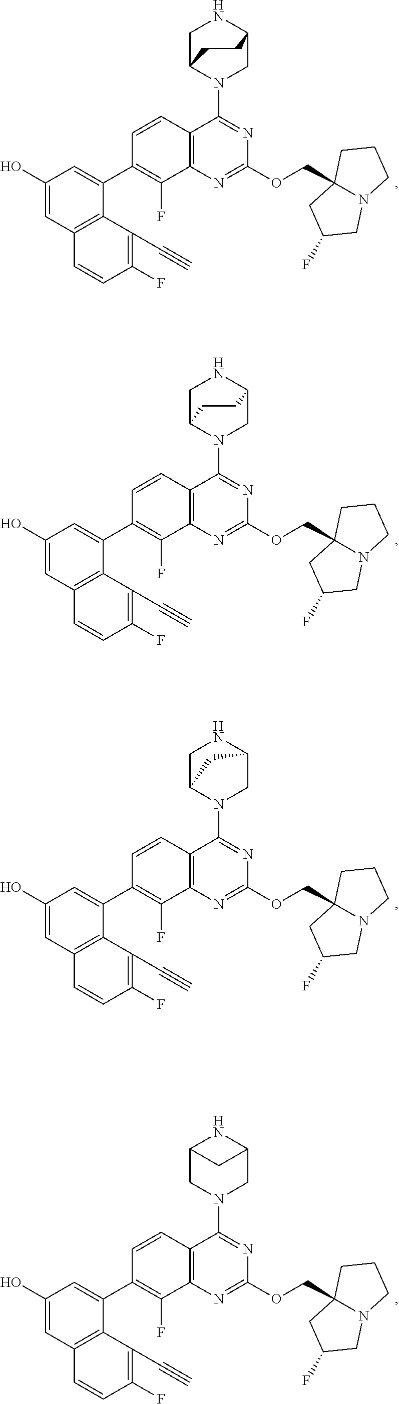 [SMILES: C#Cc1c(F)ccc2cc(O)cc(-c3ccc4c(N5C[C@@H]6CC[C@H]5CN6)nc(OC[C@@]56CCCN5C[C@H](F)C6)nc4c3F)c12 ; C#Cc1c(F)ccc2cc(O)cc(-c3ccc4c(N5C[C@H]6CC[C@@H]5CN6)nc(OC[C@@]56CCCN5C[C@H](F)C6)nc4c3F)c12 ; C#Cc1c(F)ccc2cc(O)cc(-c3ccc4c(N5C[C@H]6C[C@@H]5CN6)nc(OC[C@@]56CCCN5C[C@H](F)C6)nc4c3F)c12 ; C#Cc1c(F)ccc2cc(O)cc(-c3ccc4c(N5CC6CC(C5)N6)nc(OC[C@@]56CCCN5C[C@H](F)C6)nc4c3F)c12]

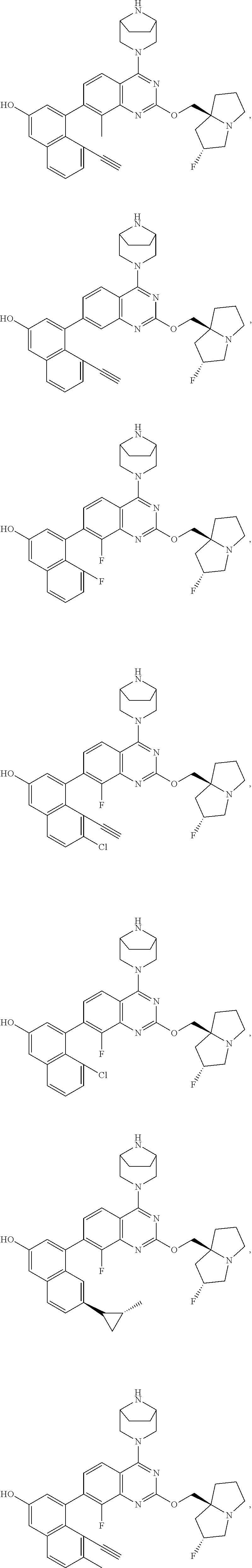

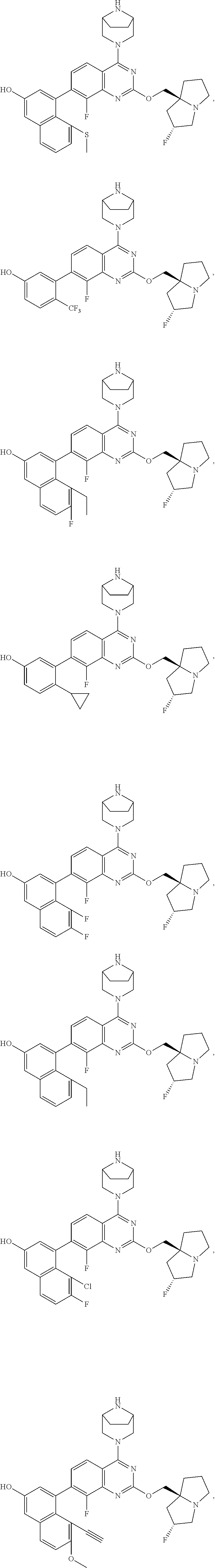

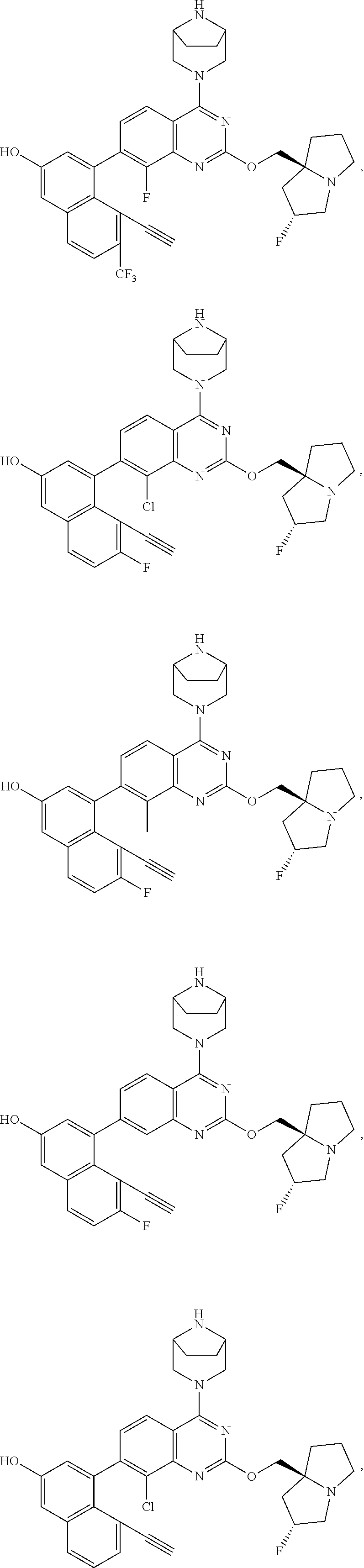 [SMILES: C#Cc1c(C(F)(F)F)ccc2cc(O)cc(-c3ccc4c(N5CC6CCC(C5)N6)nc(OC[C@@]56CCCN5C[C@H](F)C6)nc4c3F)c12 ; C#Cc1c(F)ccc2cc(O)cc(-c3ccc4c(N5CC6CCC(C5)N6)nc(OC[C@@]56CCCN5C[C@H](F)C6)nc4c3Cl)c12 ; C#Cc1c(F)ccc2cc(O)cc(-c3ccc4c(N5CC6CCC(C5)N6)nc(OC[C@@]56CCCN5C[C@H](F)C6)nc4c3C)c12 ; C#Cc1c(F)ccc2cc(O)cc(-c3ccc4c(N5CC6CCC(C5)N6)nc(OC[C@@]56CCCN5C[C@H](F)C6)nc4c3)c12 ; C#Cc1cccc2cc(O)cc(-c3ccc4c(N5CC6CCC(C5)N6)nc(OC[C@@]56CCCN5C[C@H](F)C6)nc4c3Cl)c12]

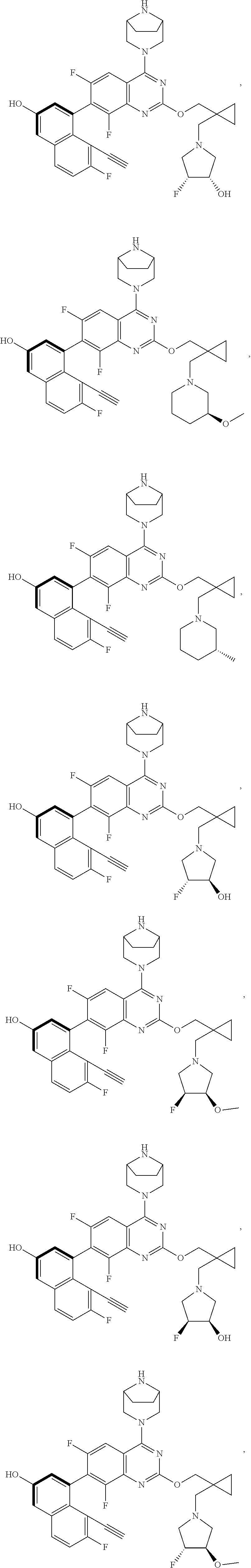

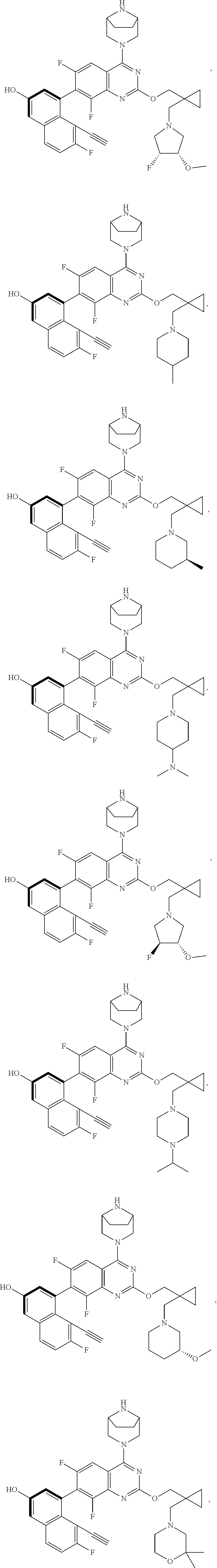

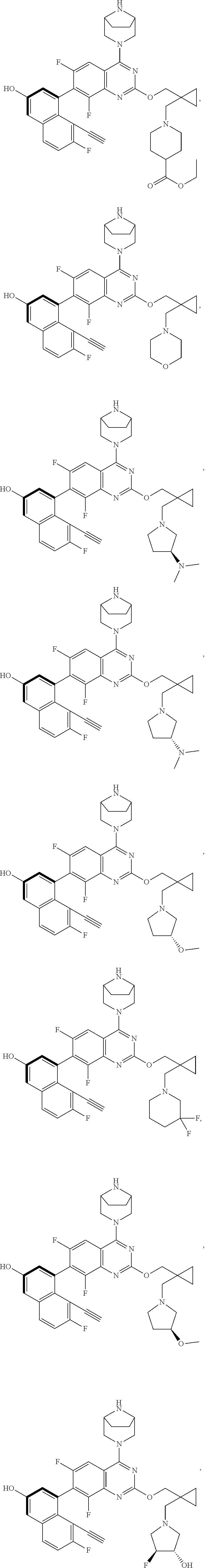

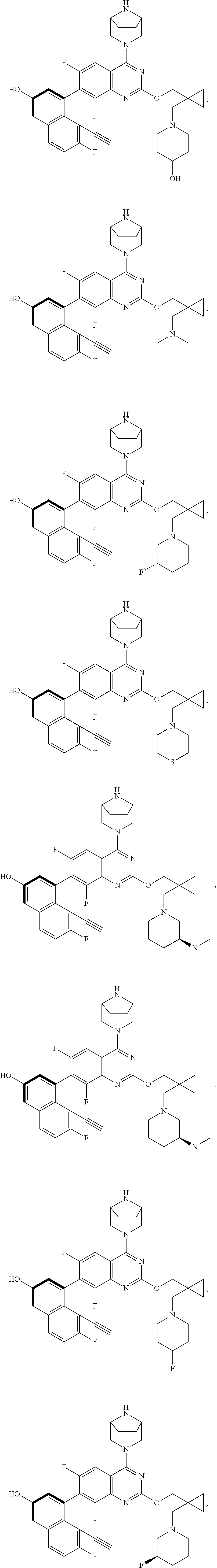

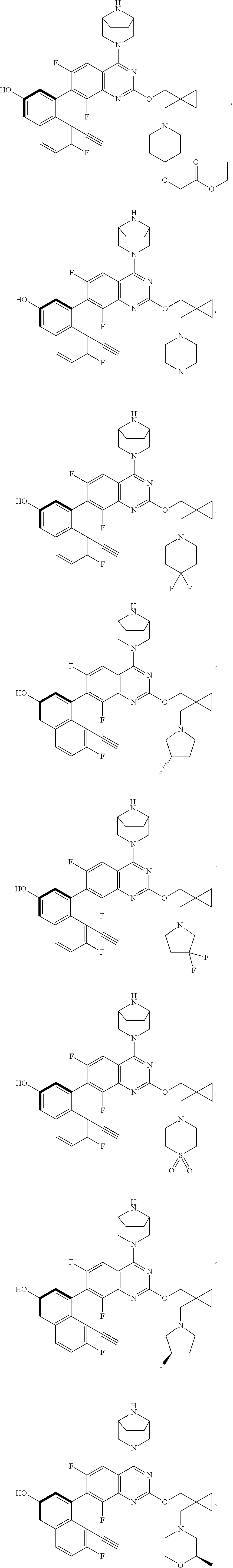

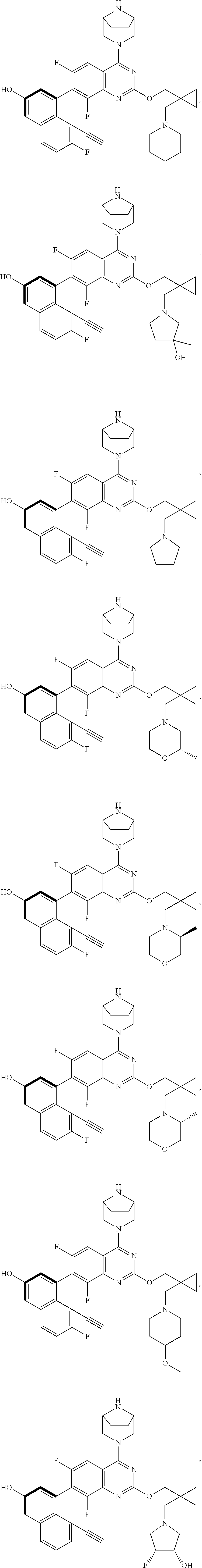

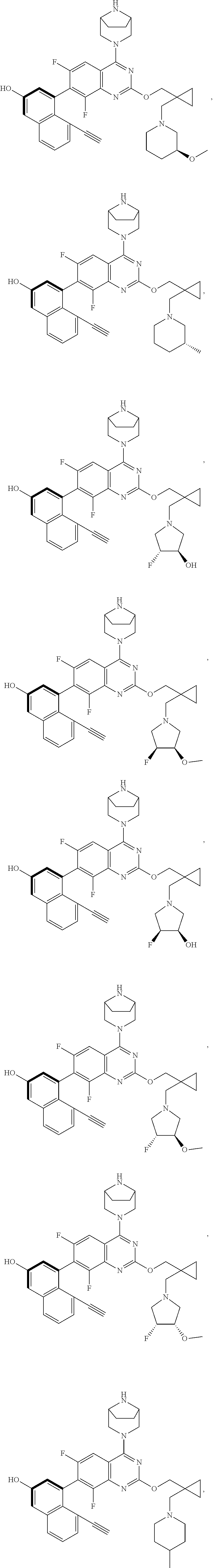

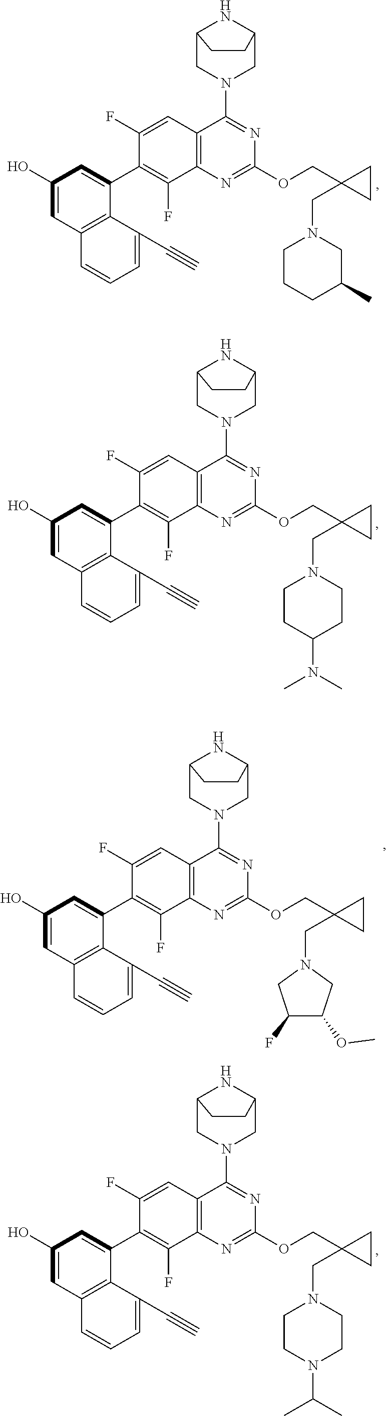

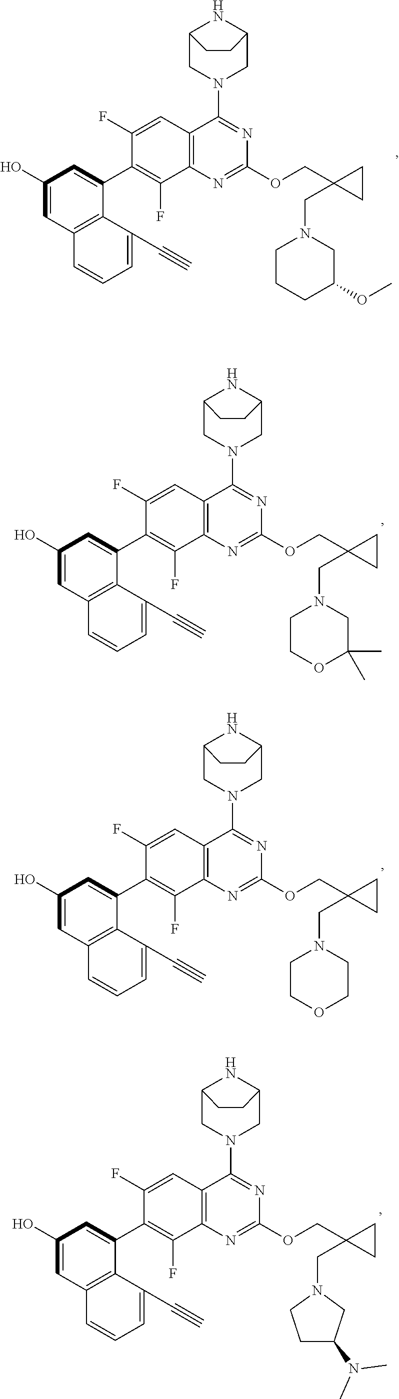

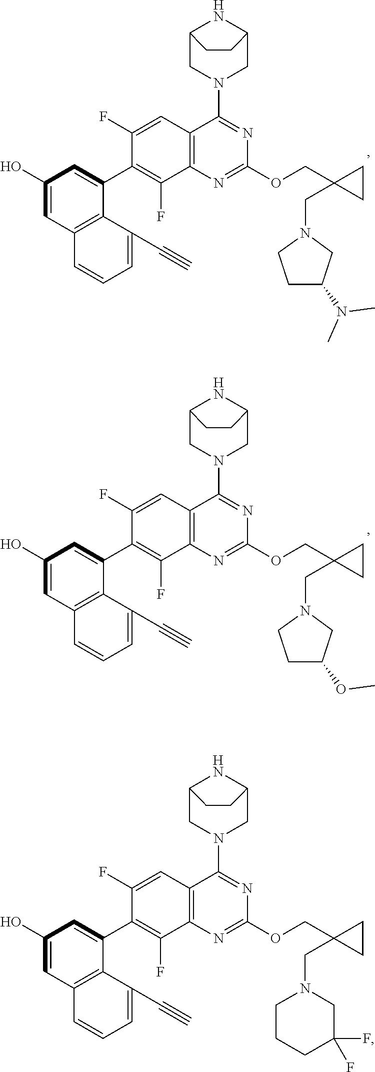

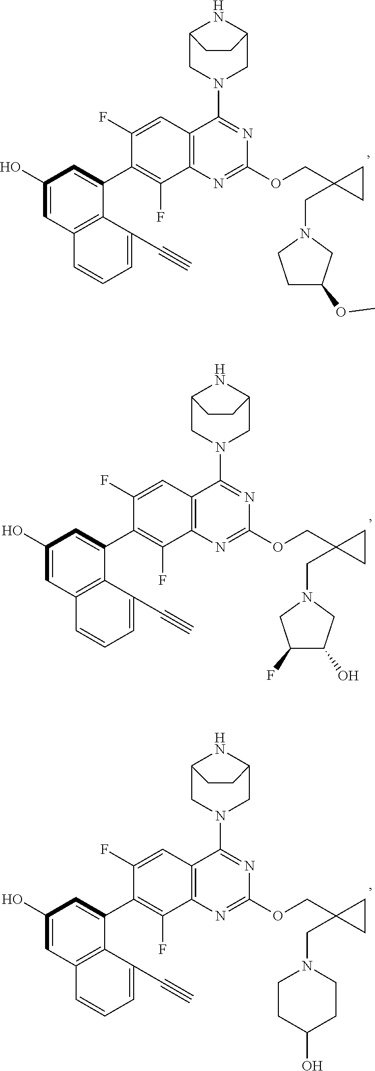

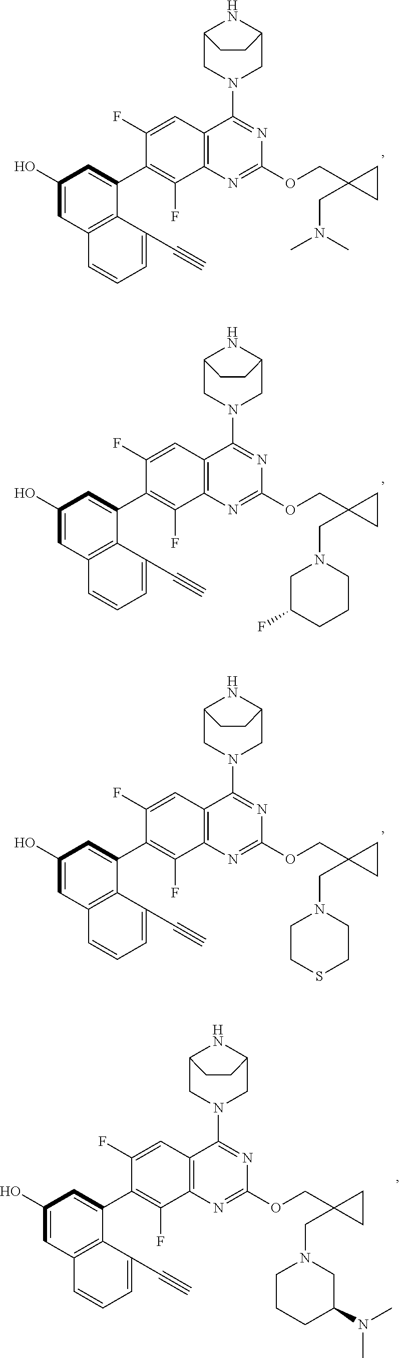

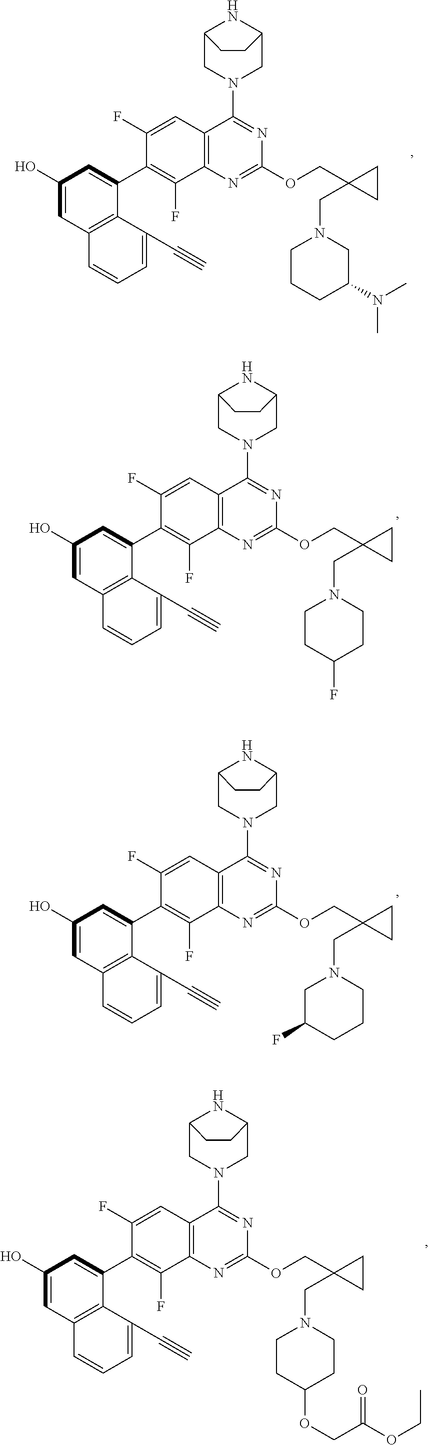

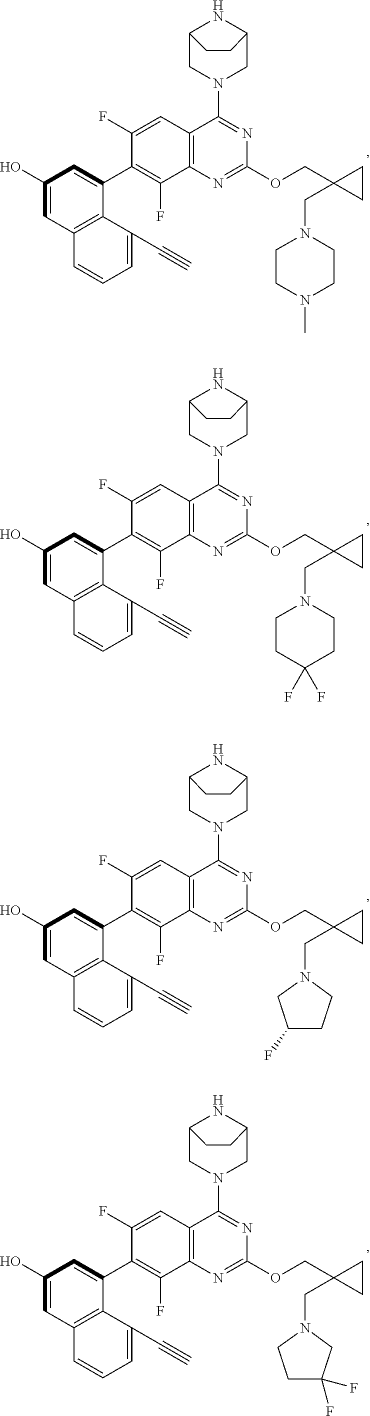

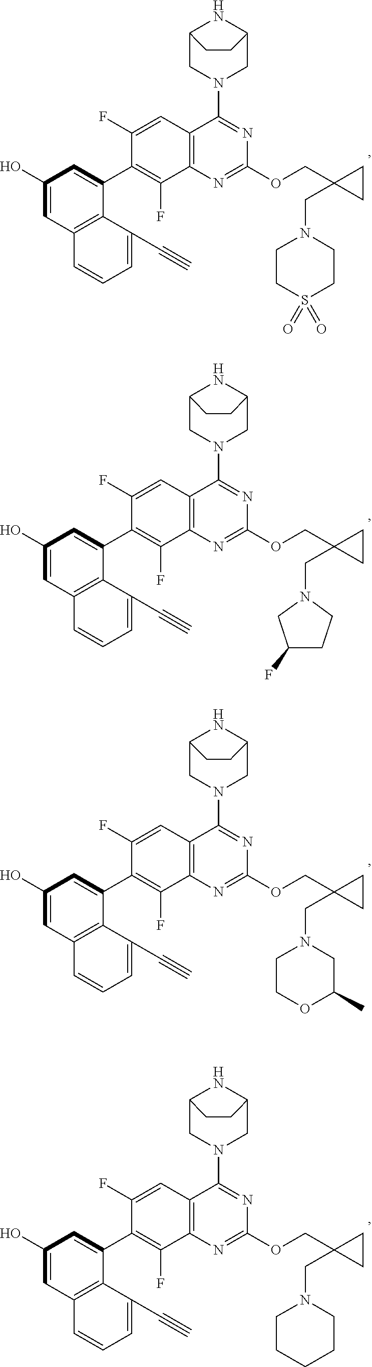

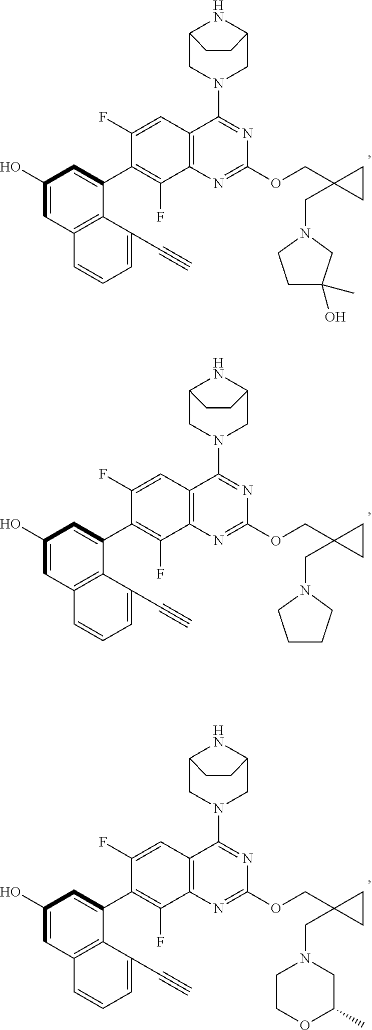

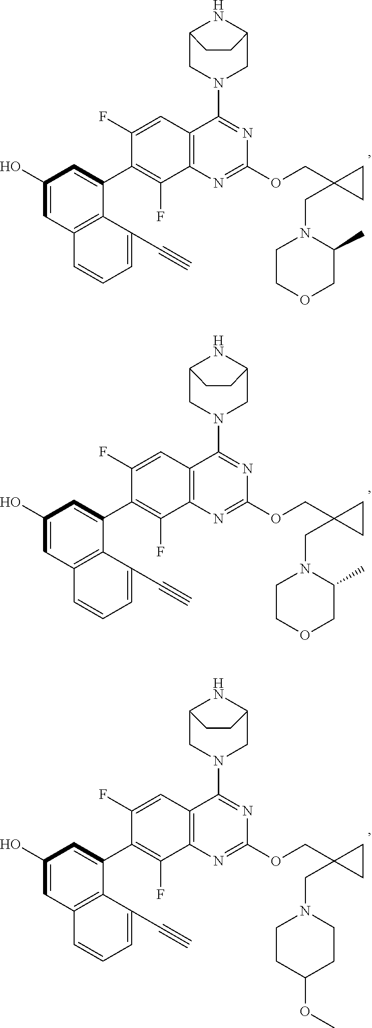

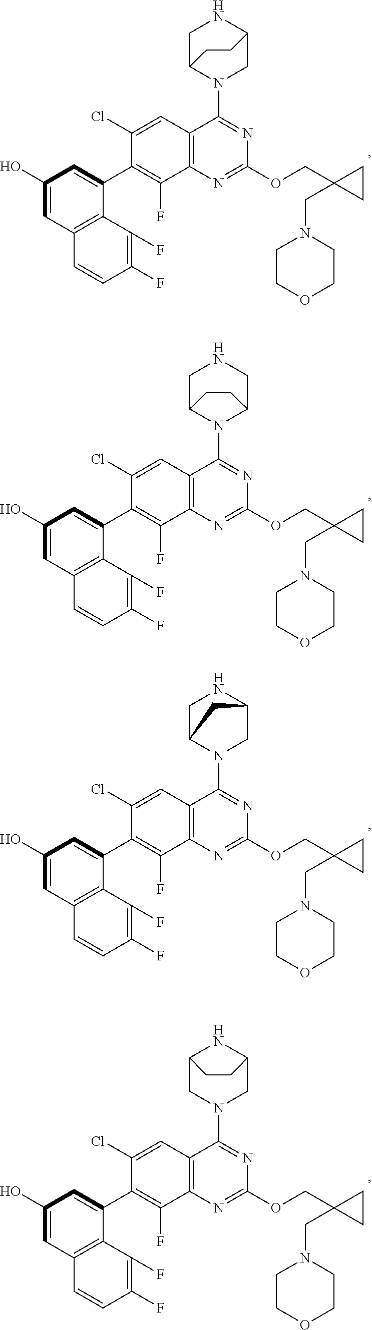

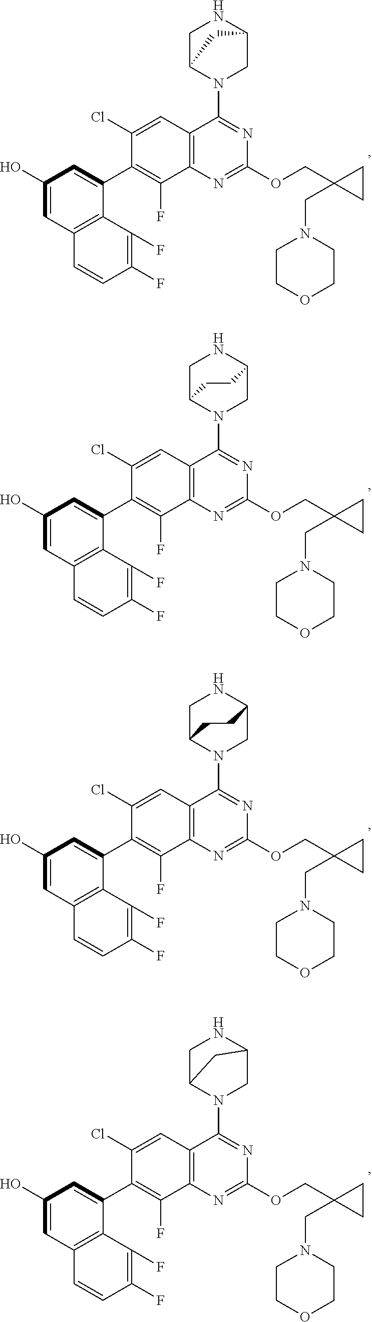

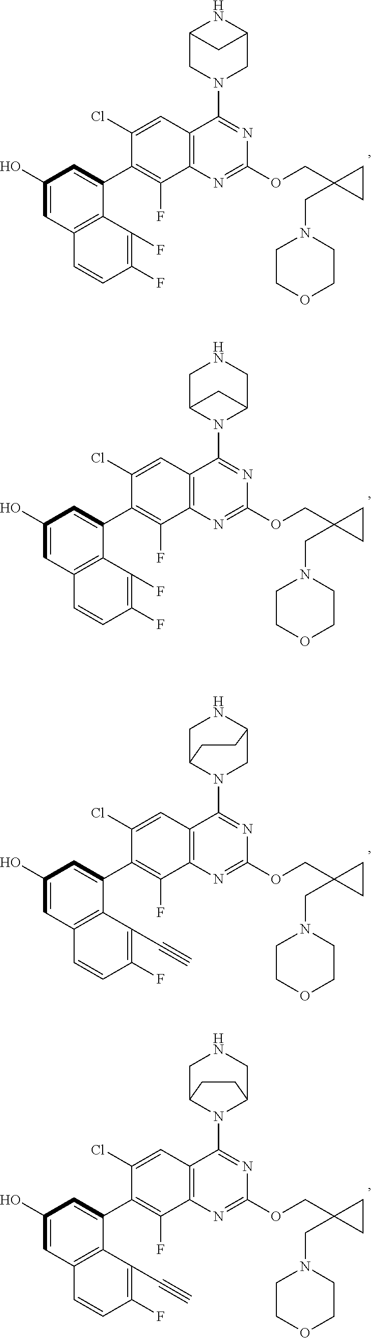

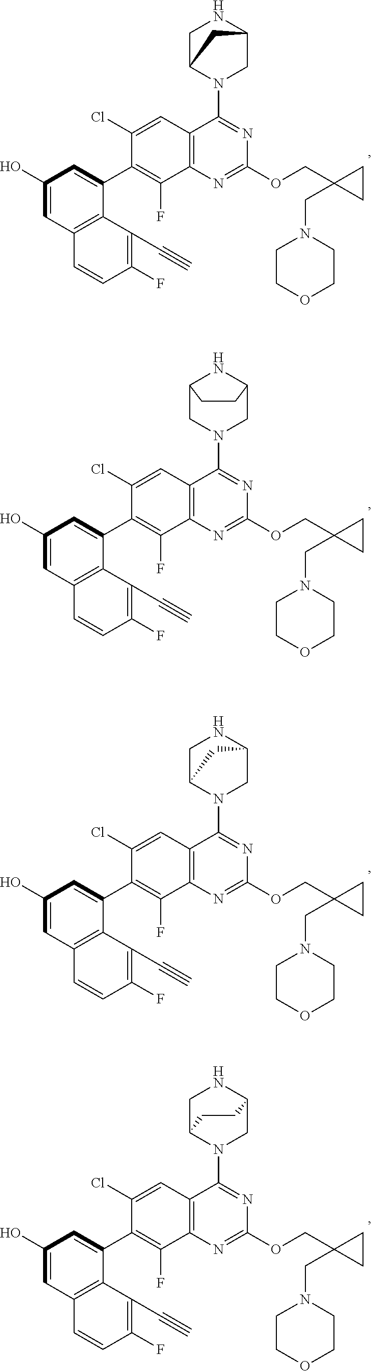

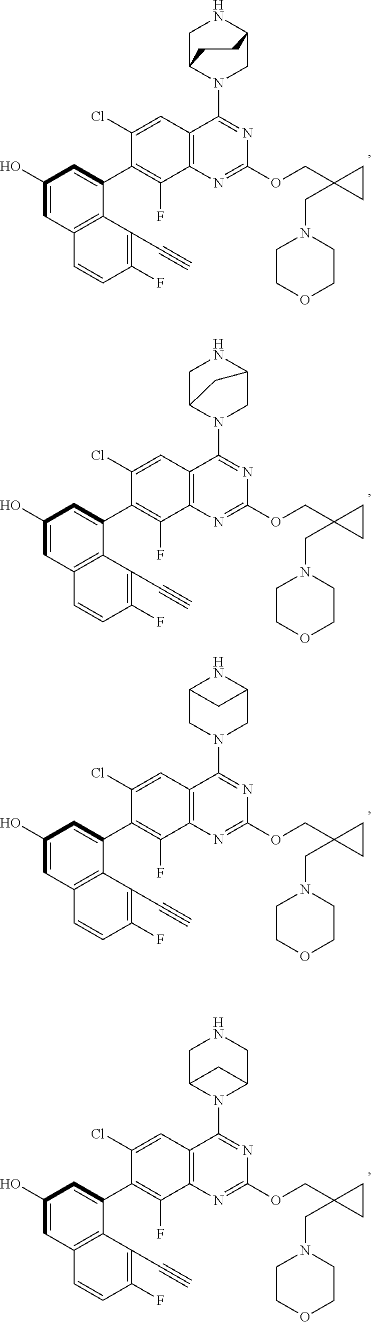

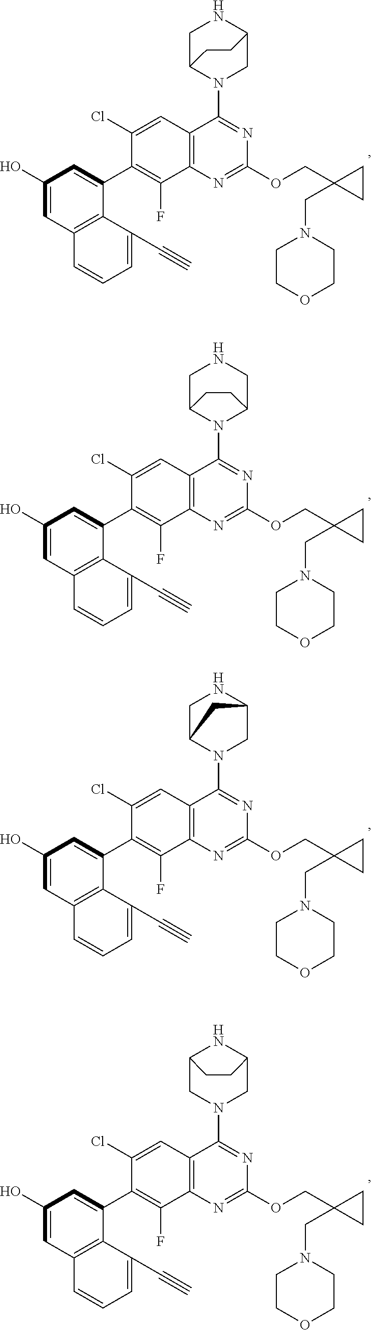

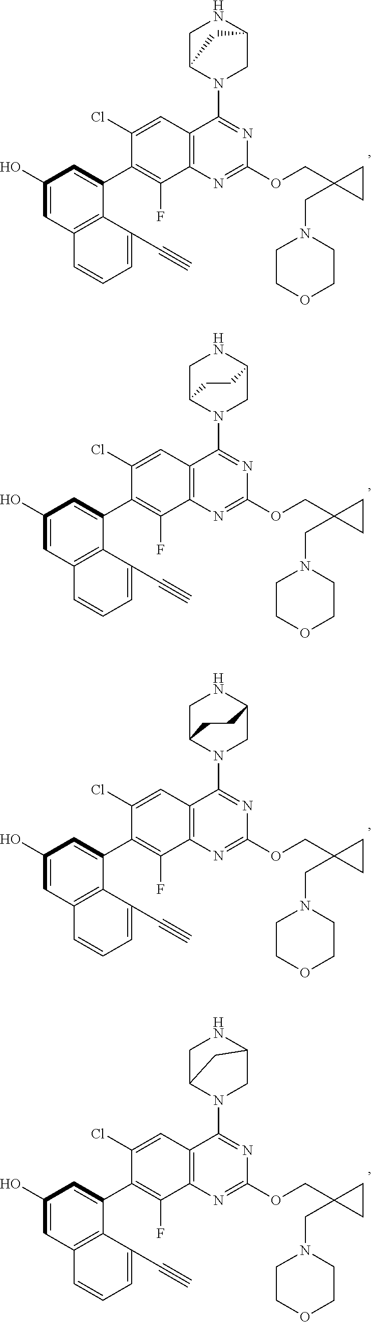

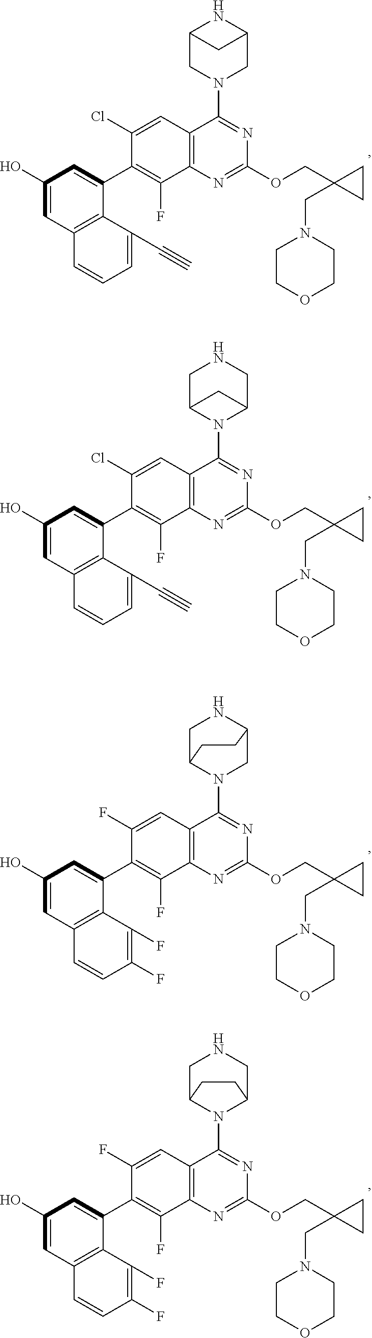

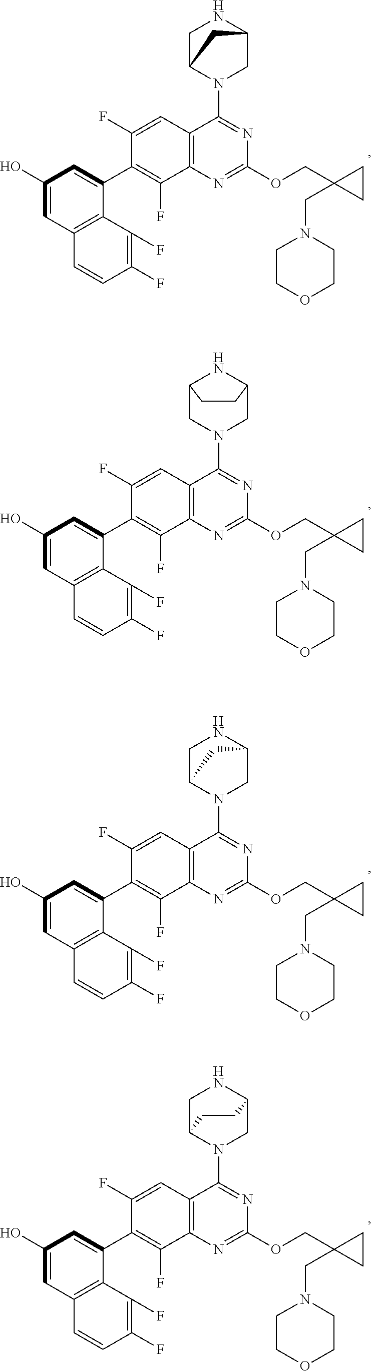

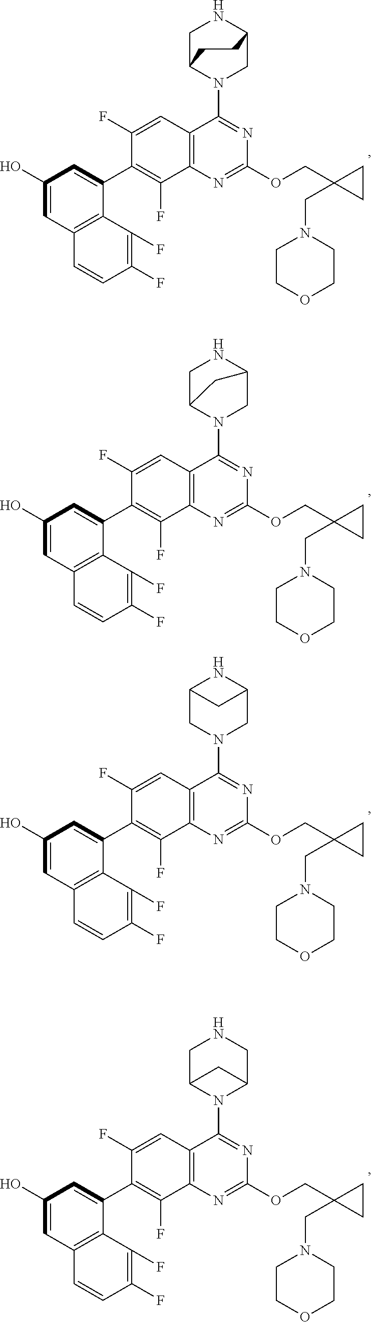

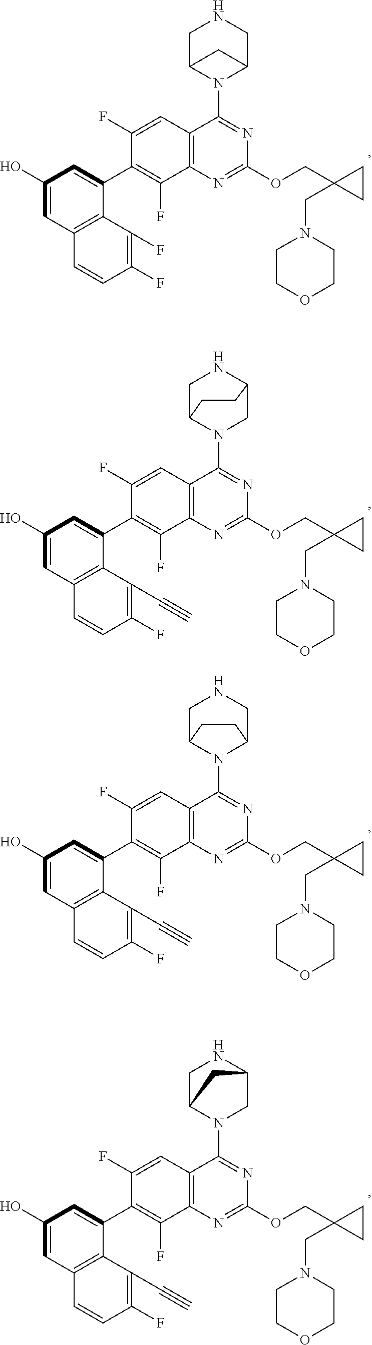

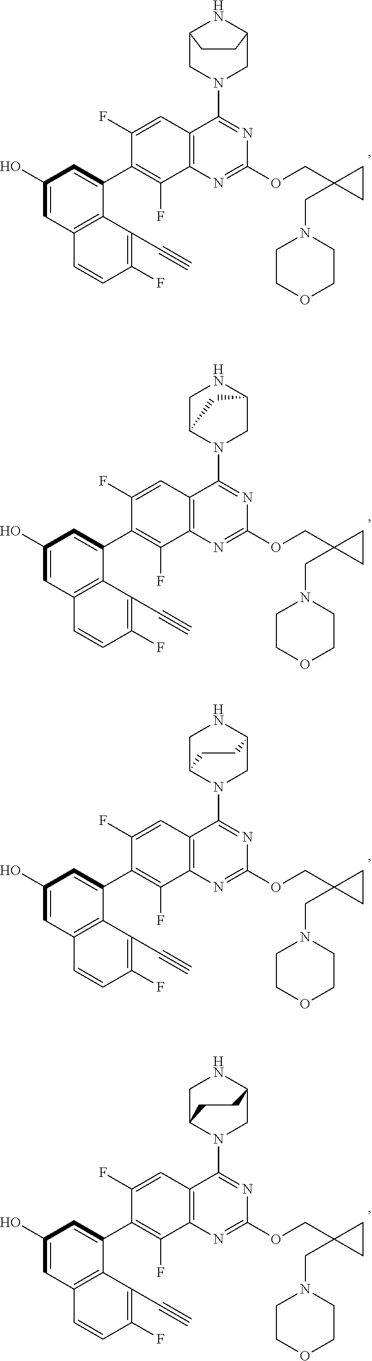

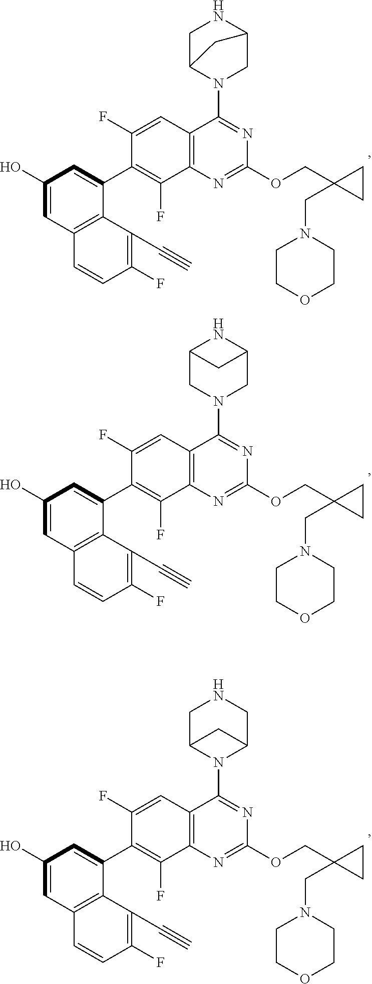

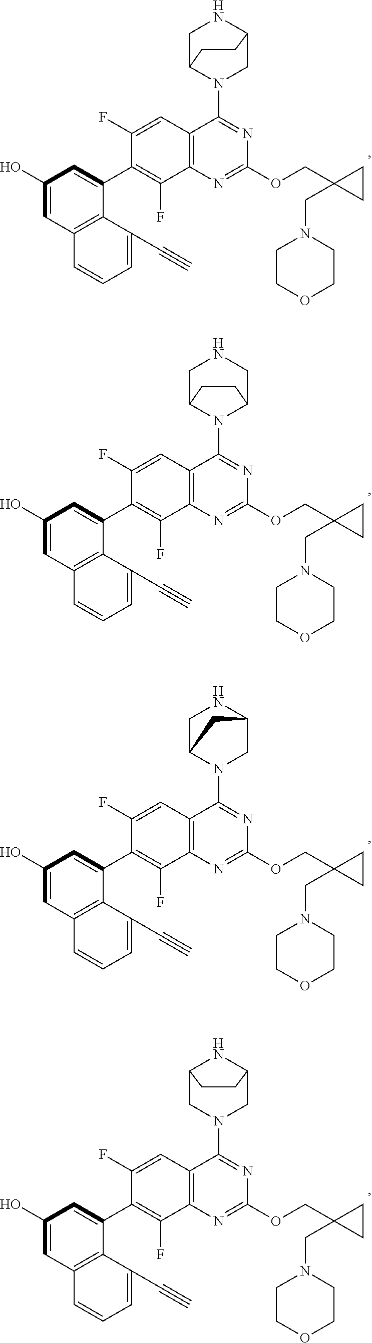

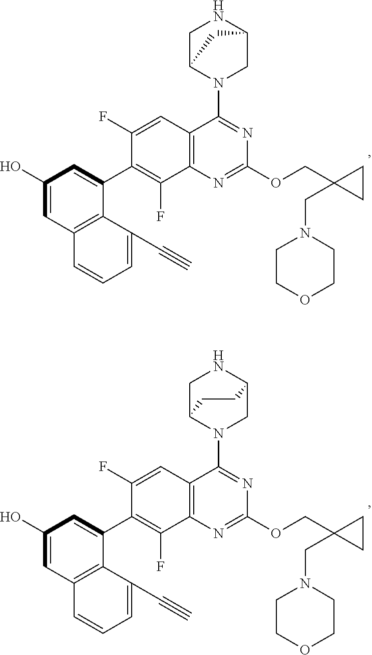

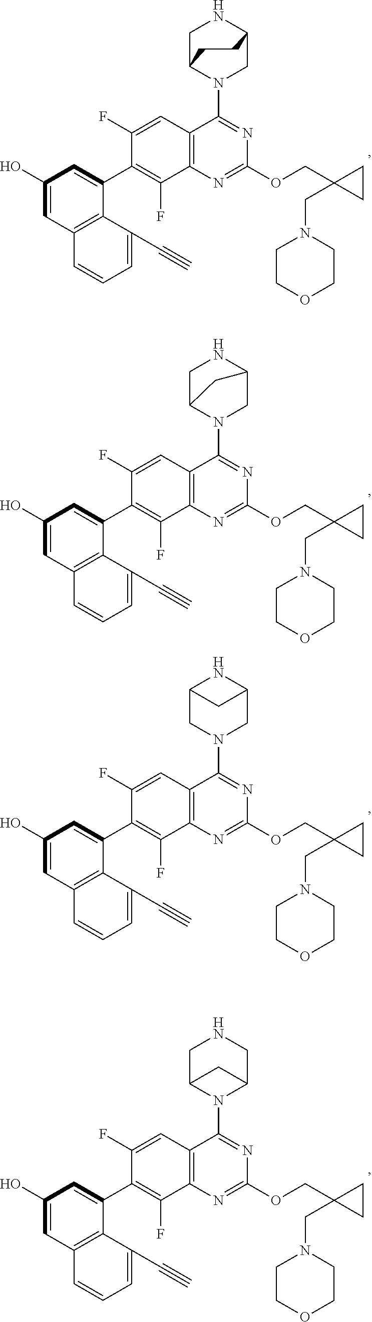

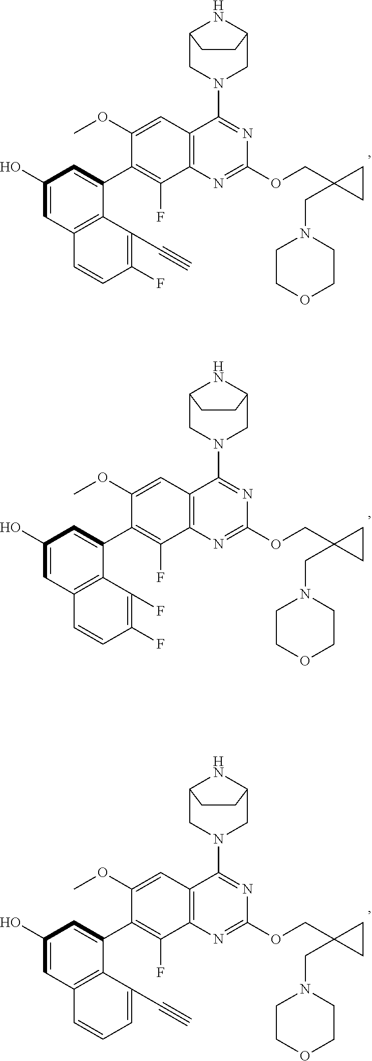

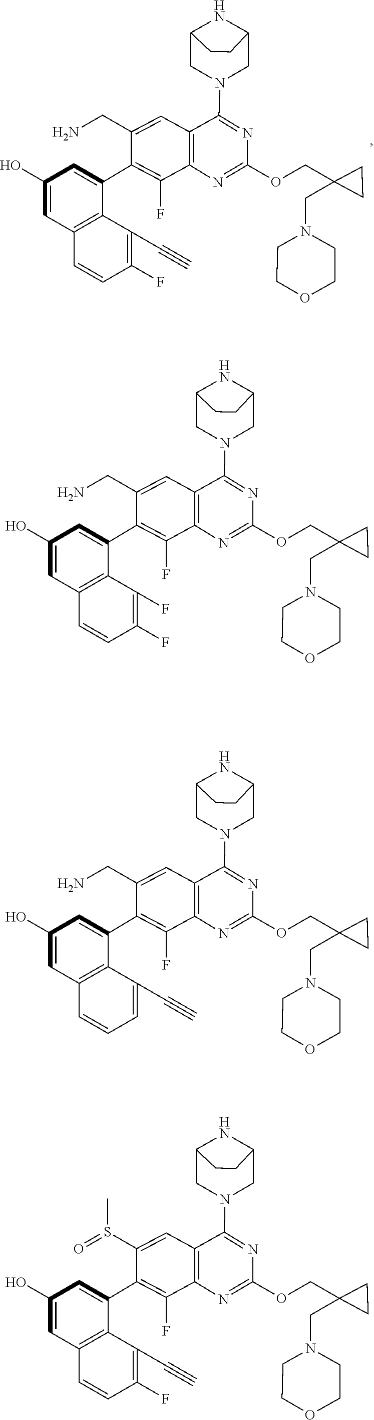

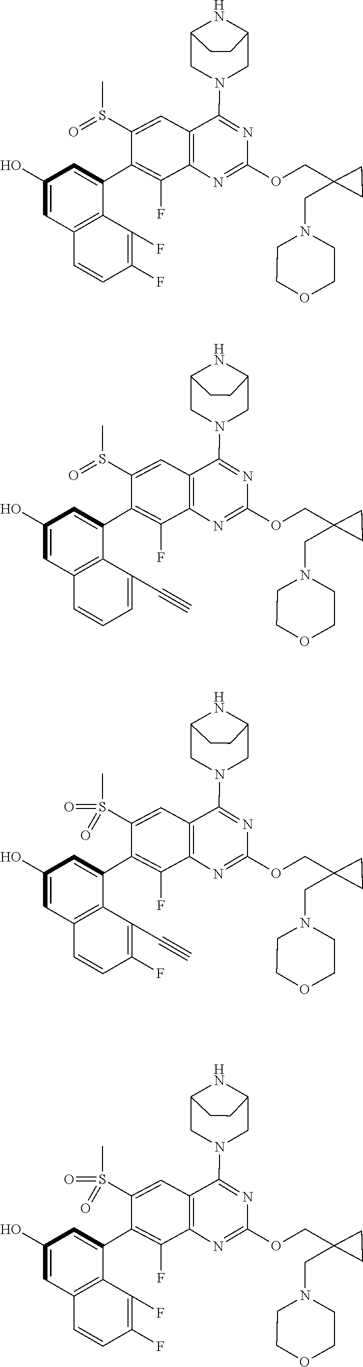

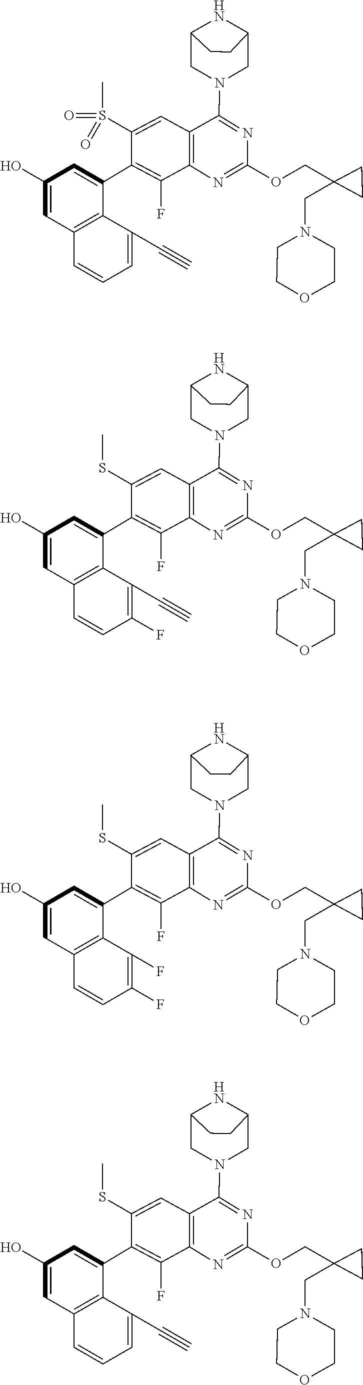

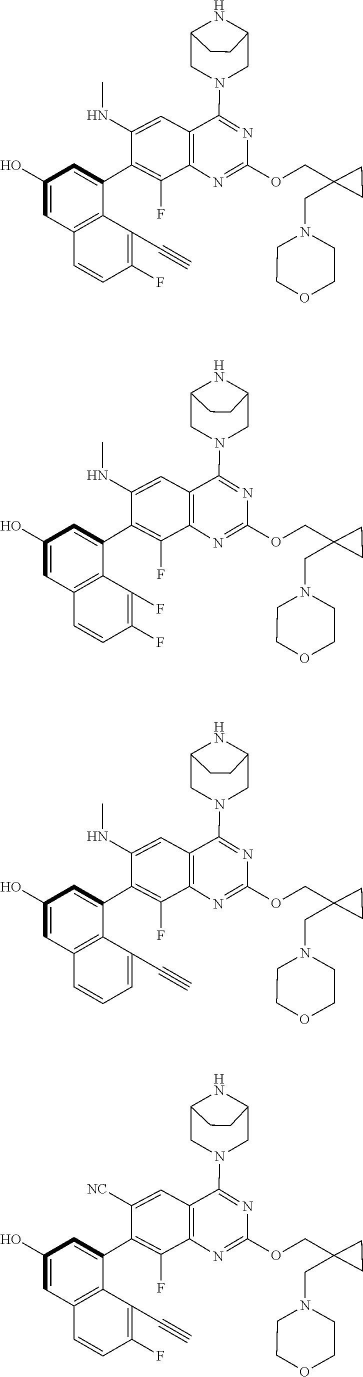

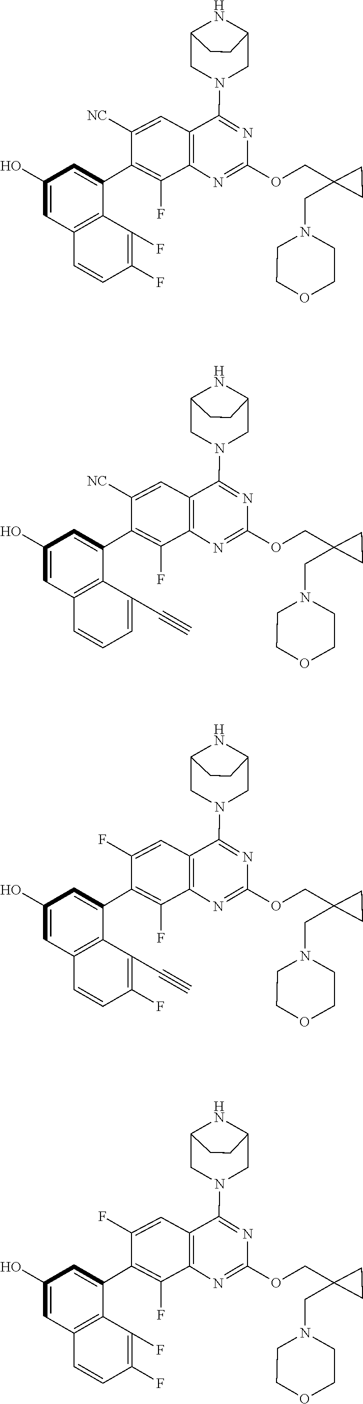

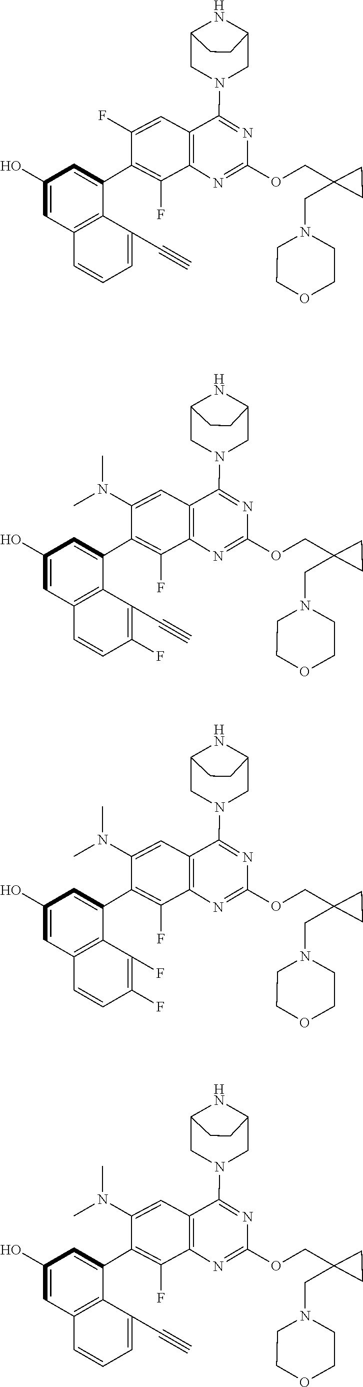

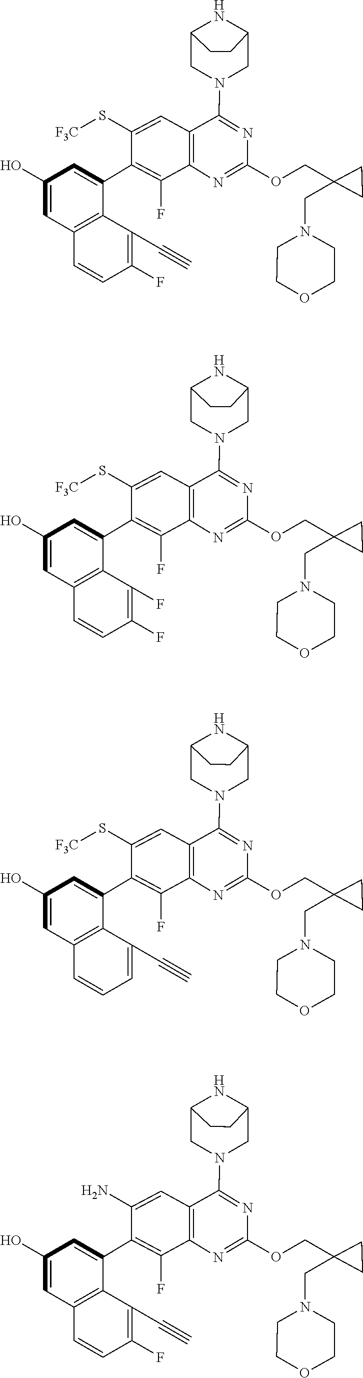

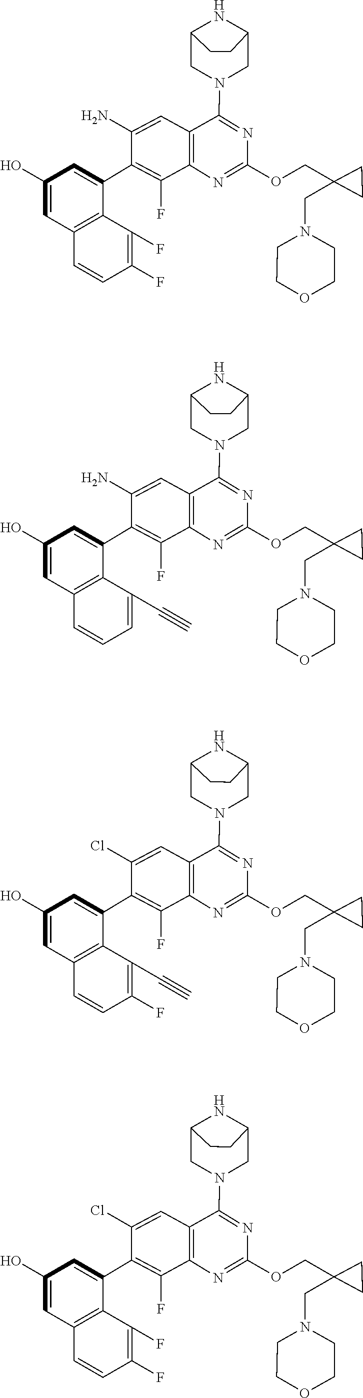

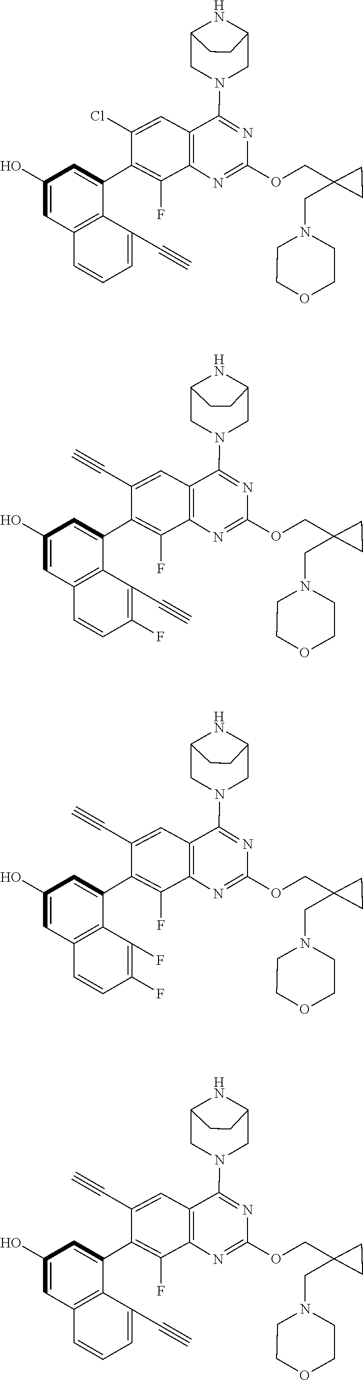

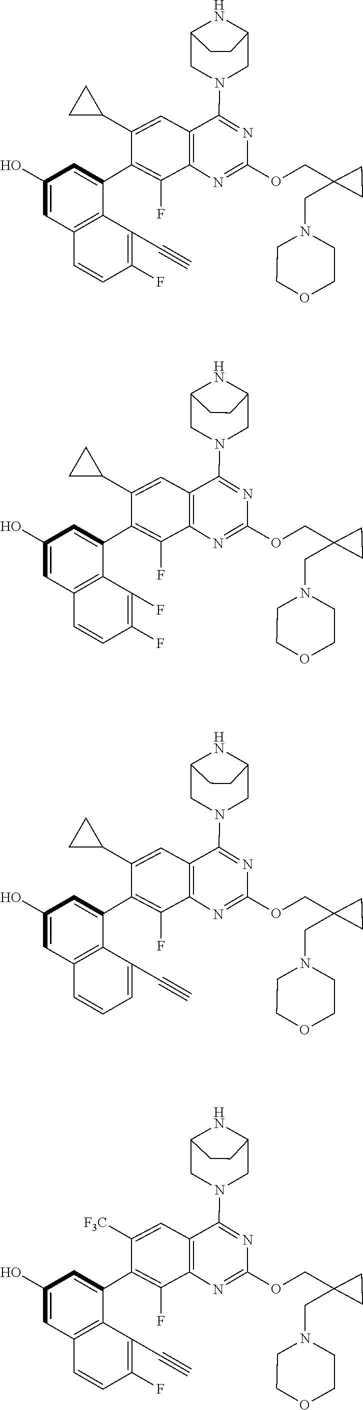

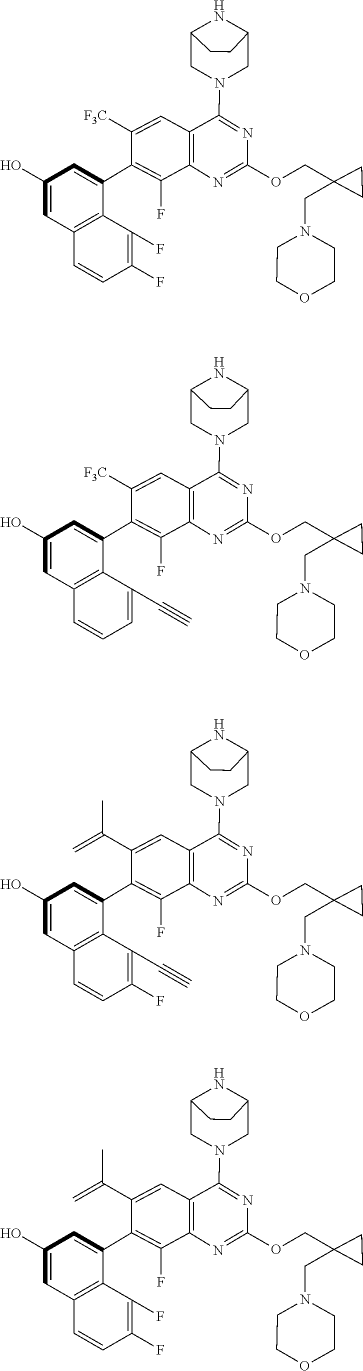

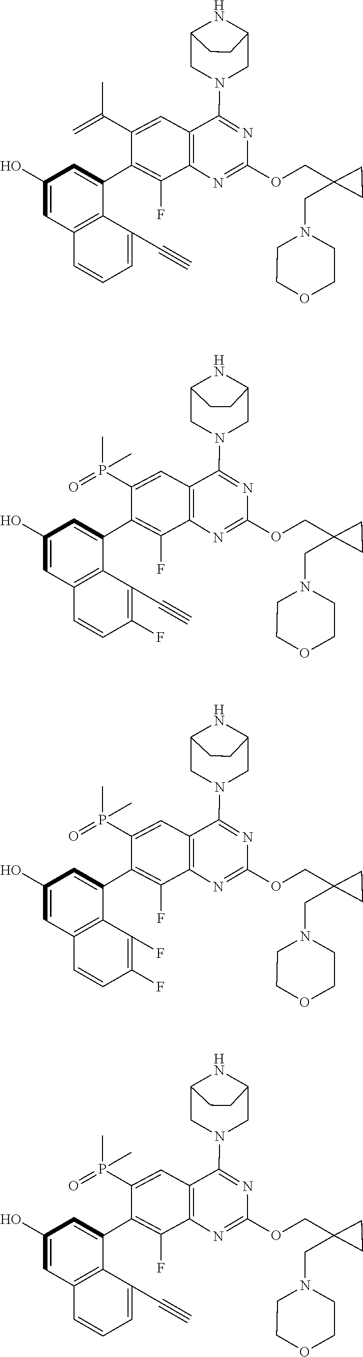

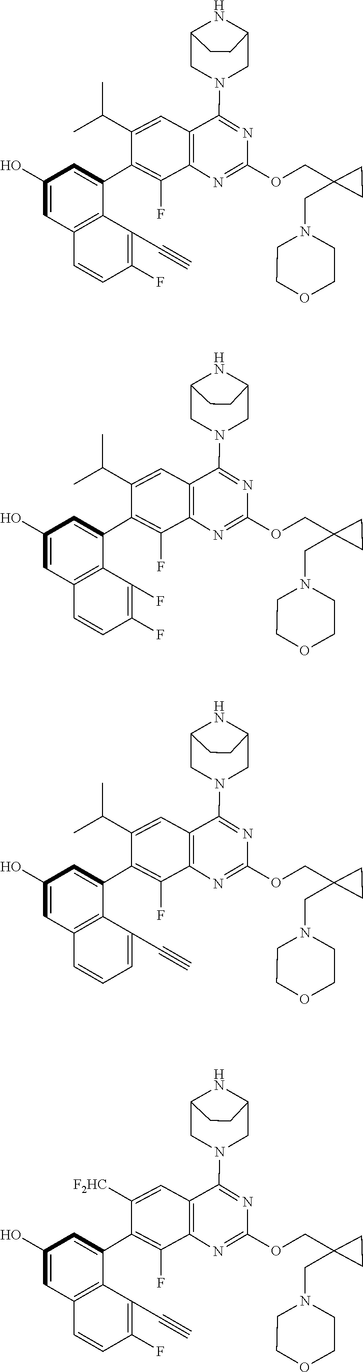

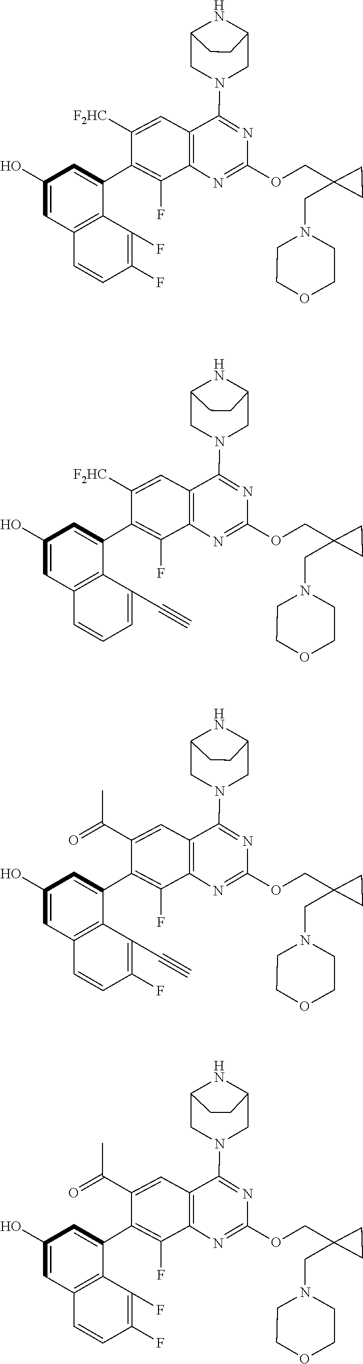

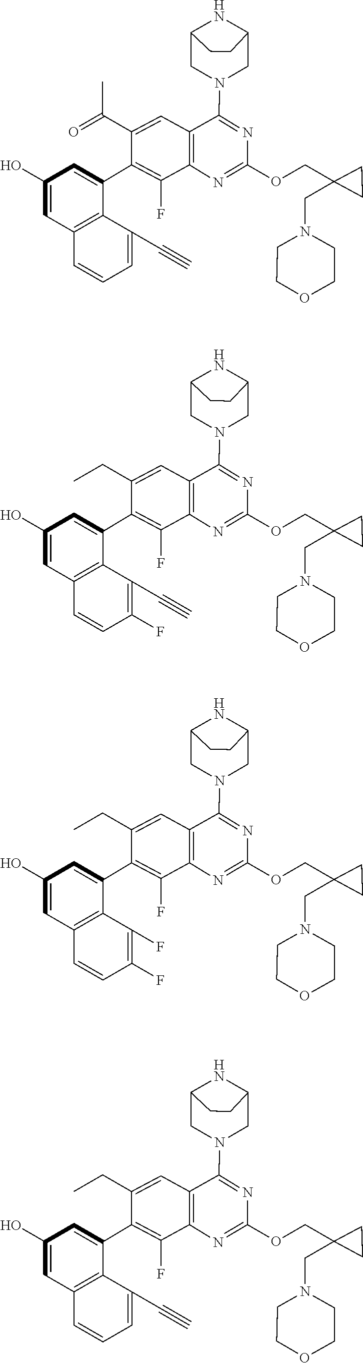

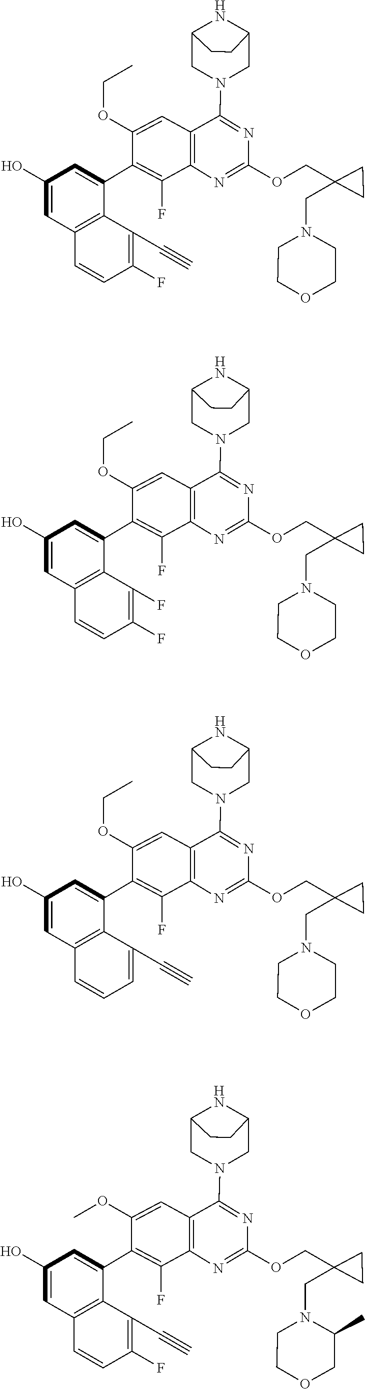

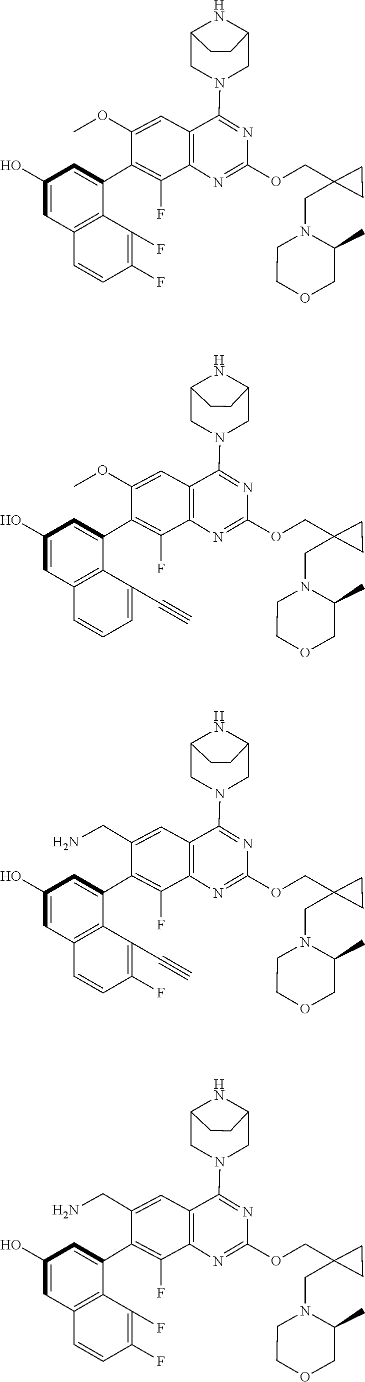

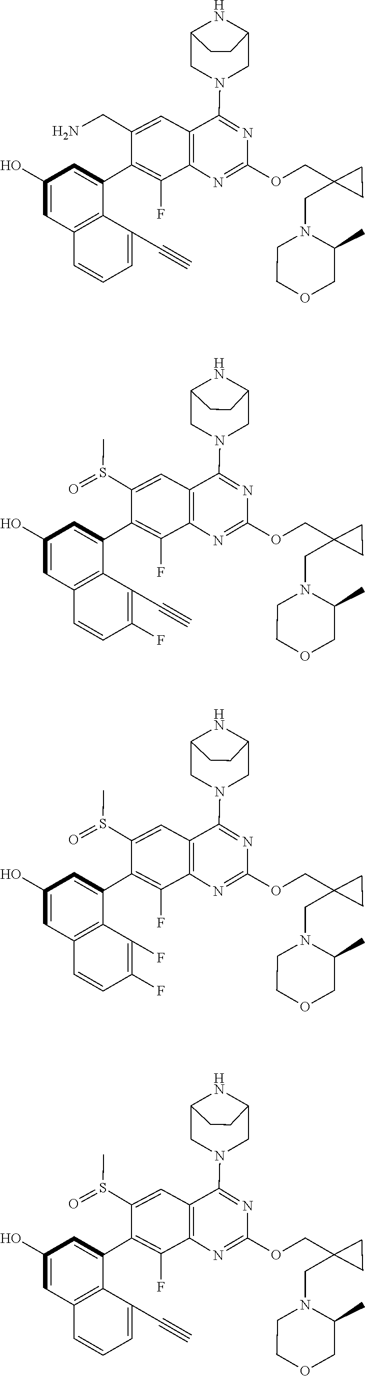

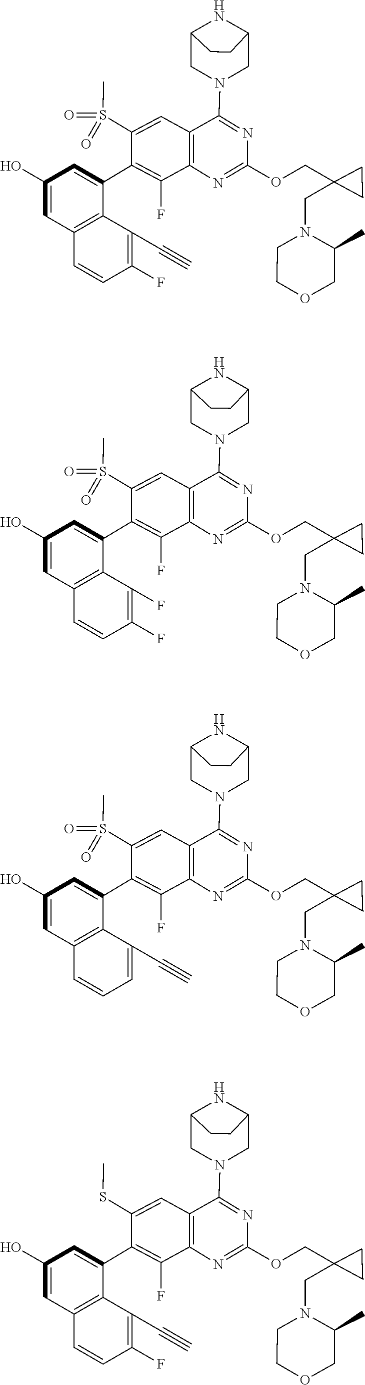

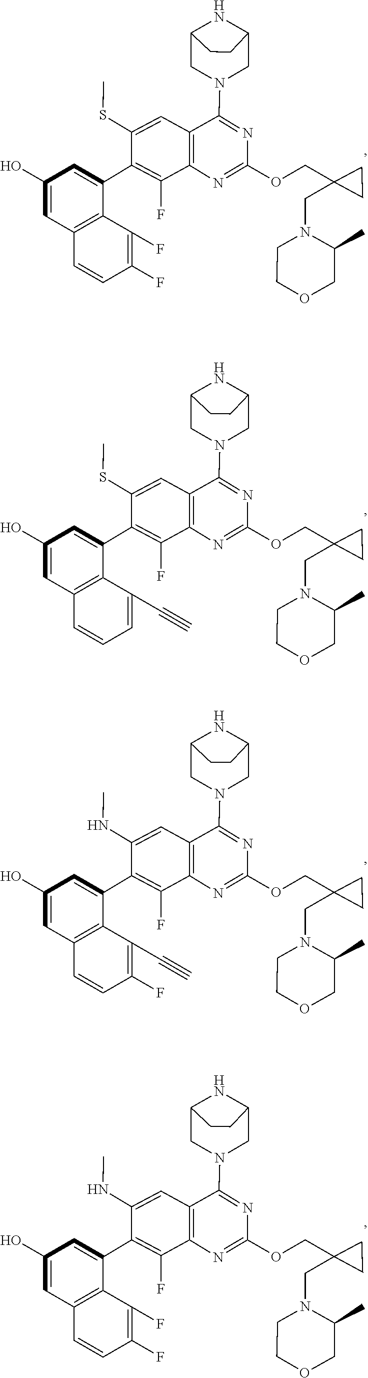

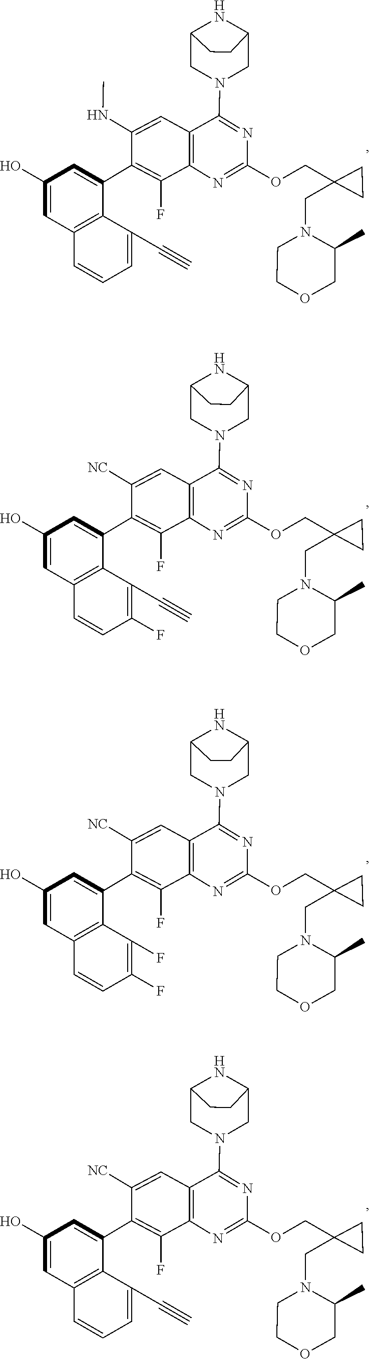

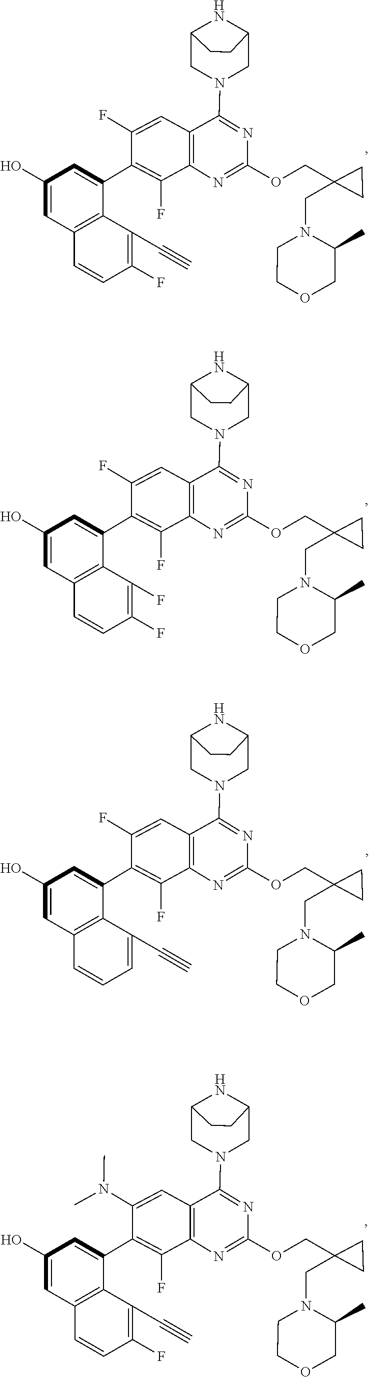

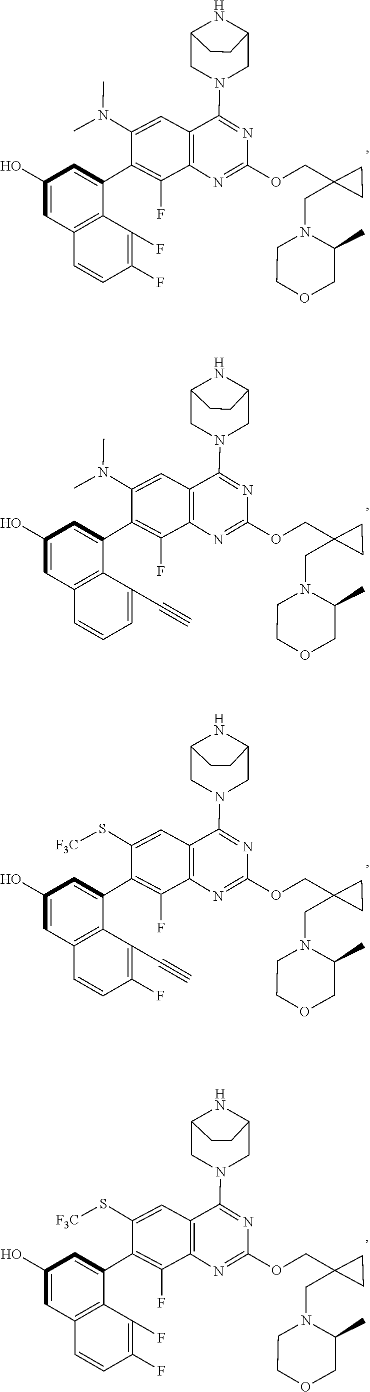

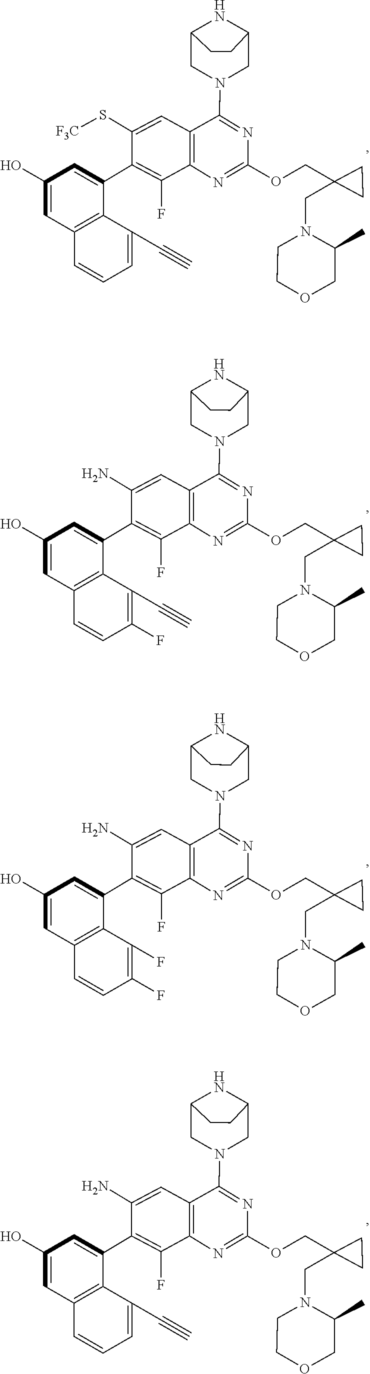

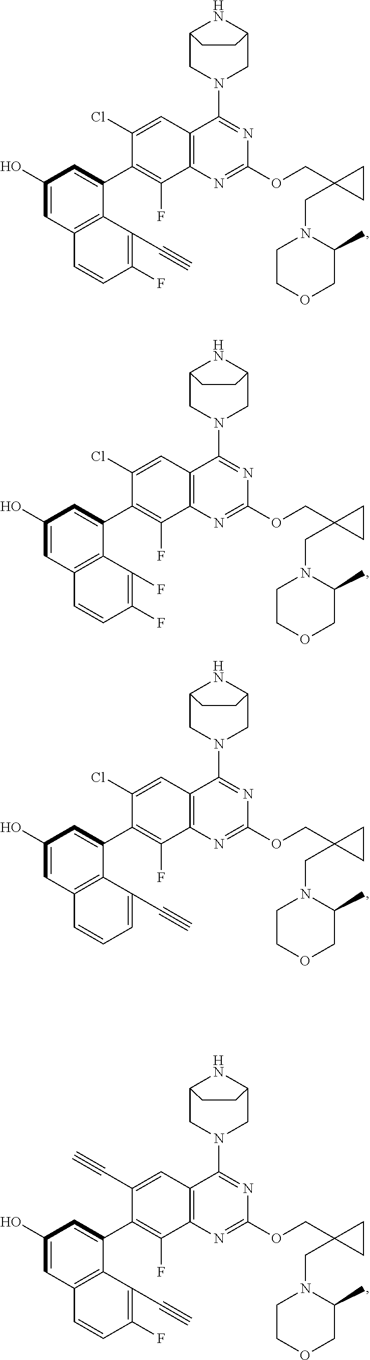

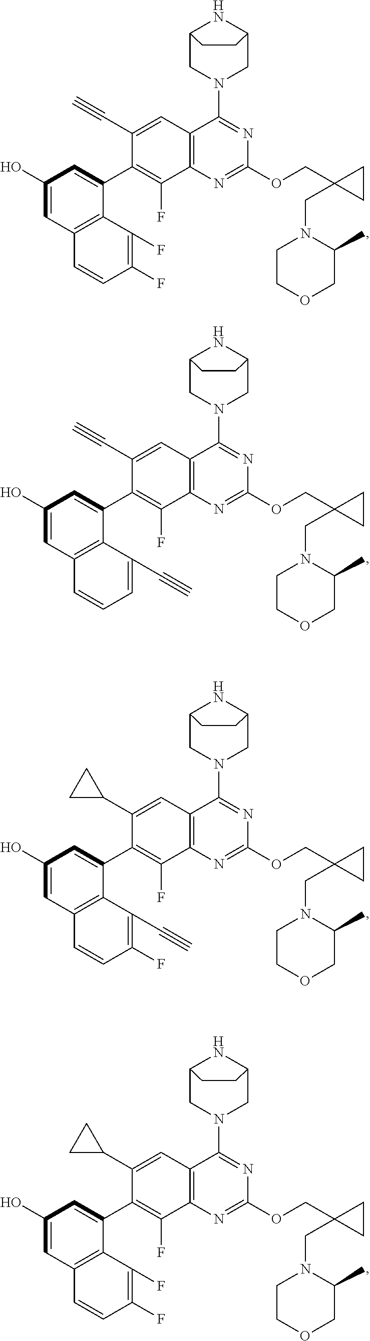

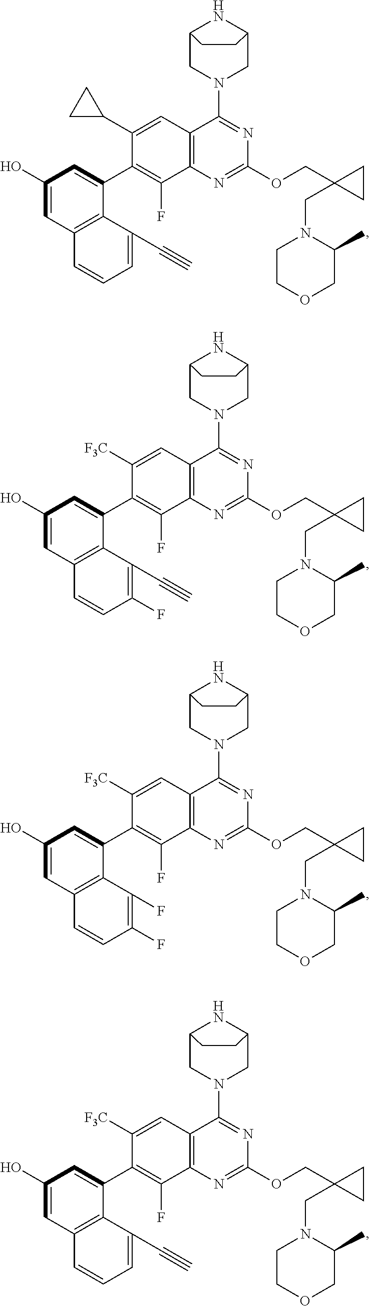

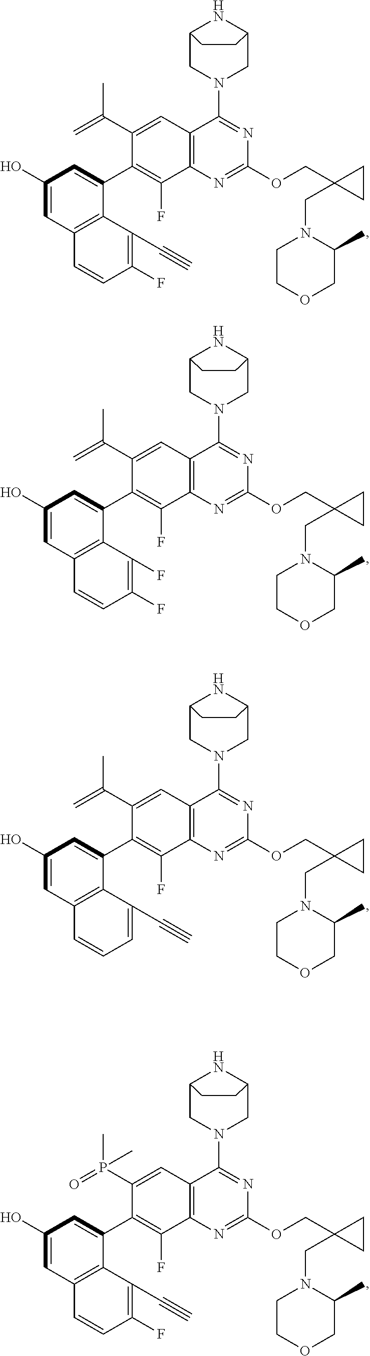

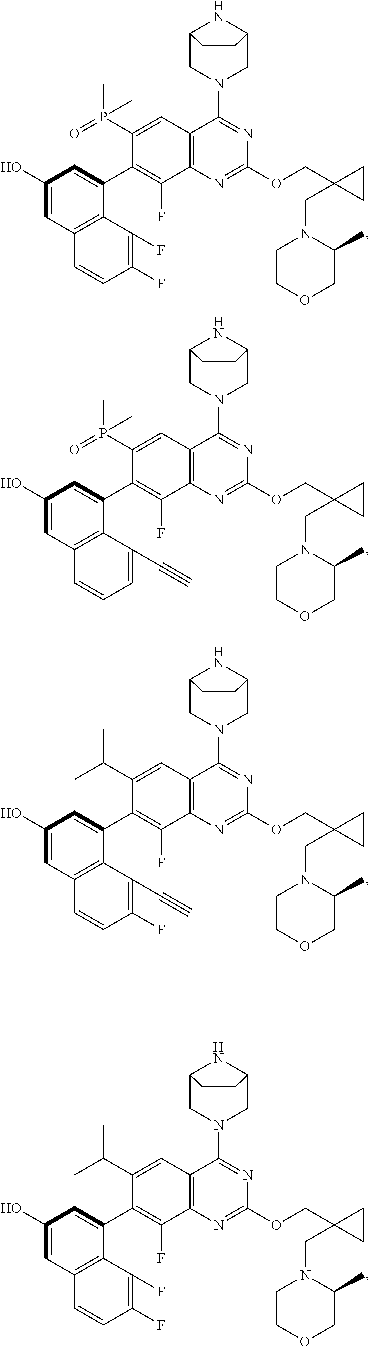

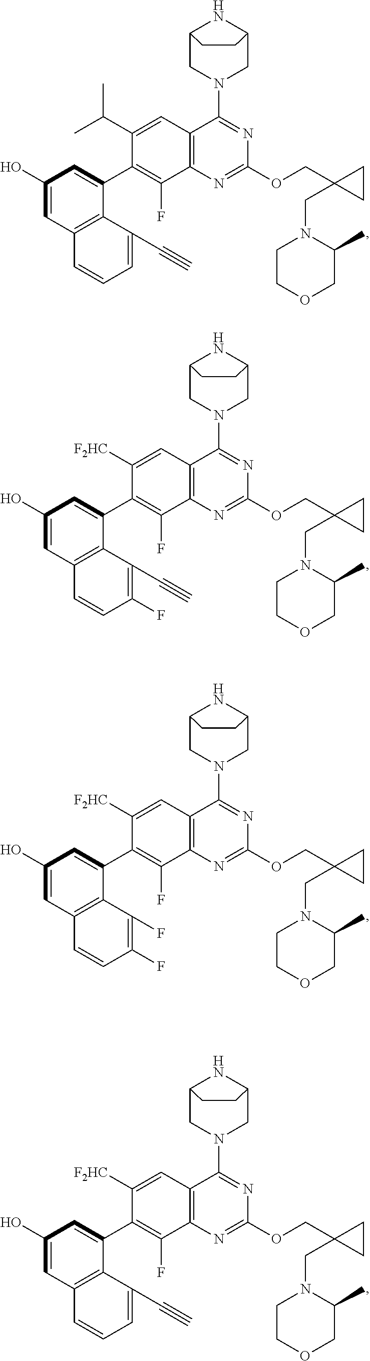

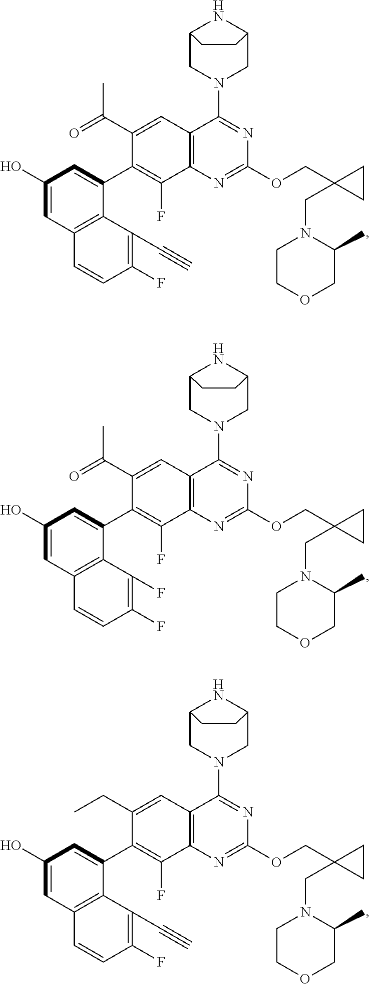

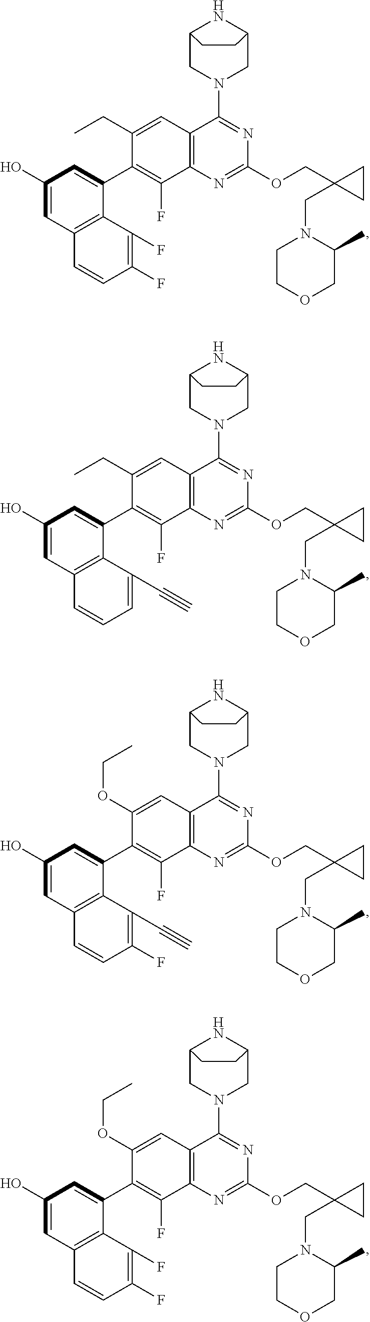

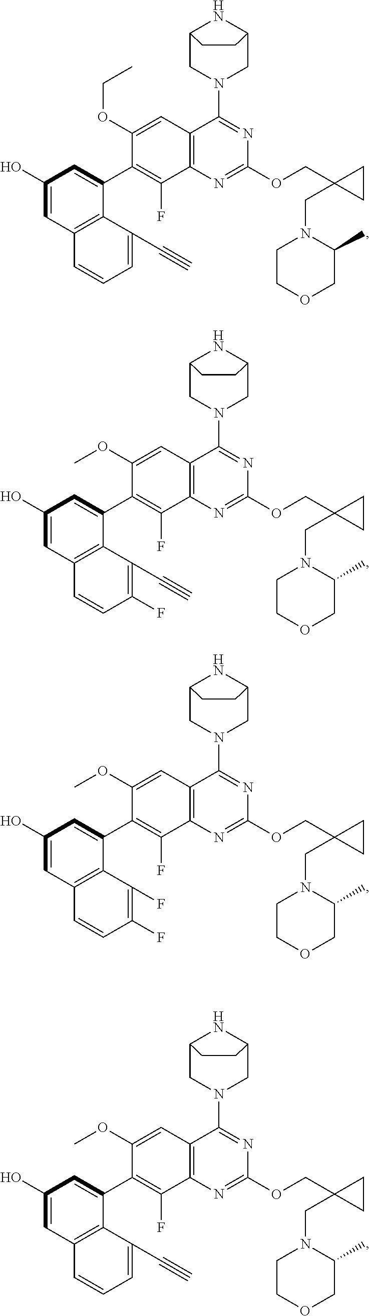

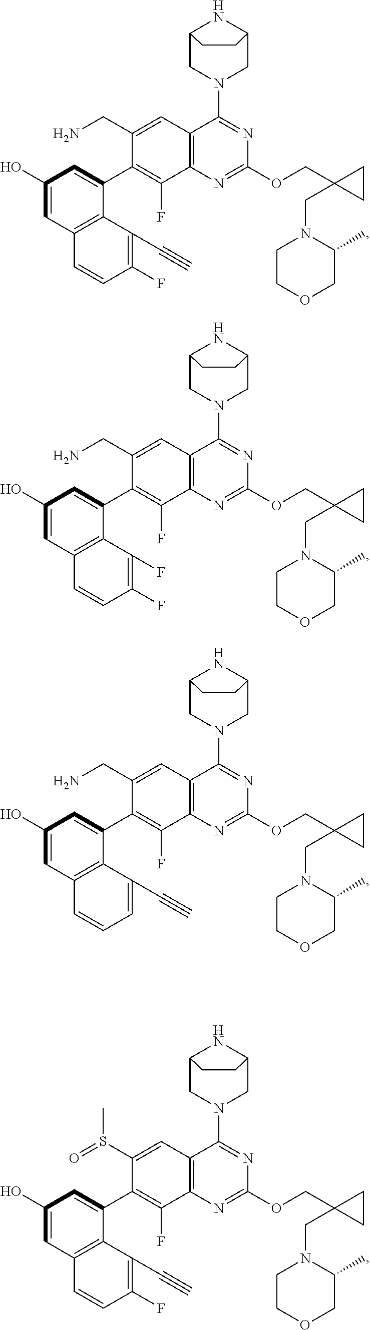

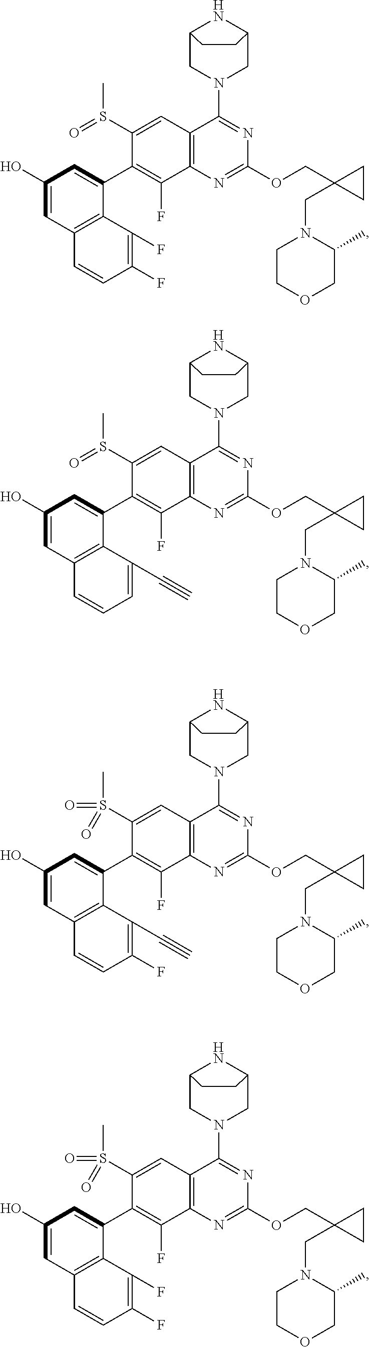

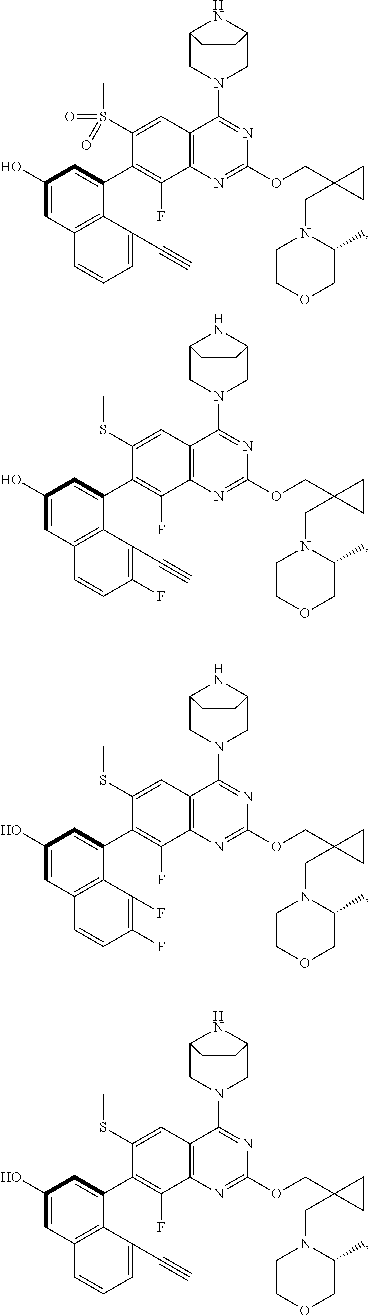

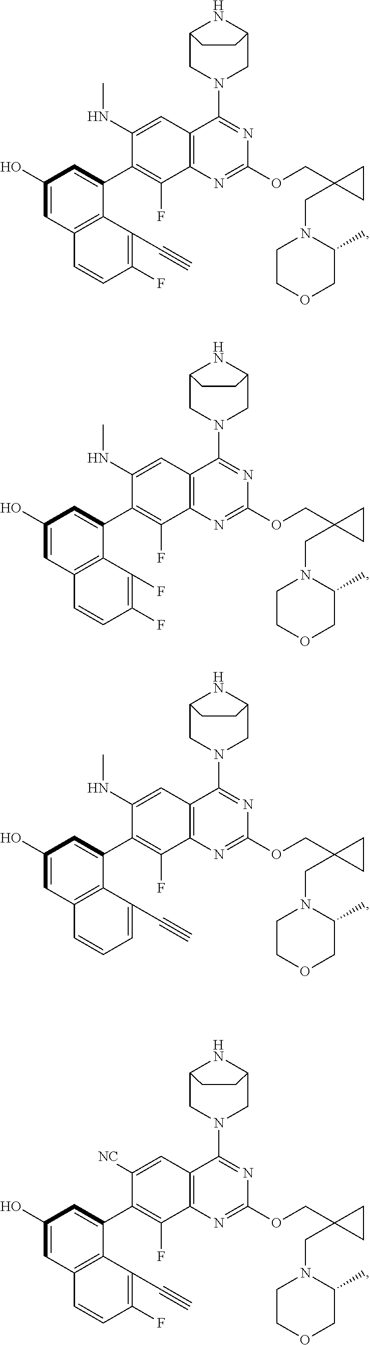

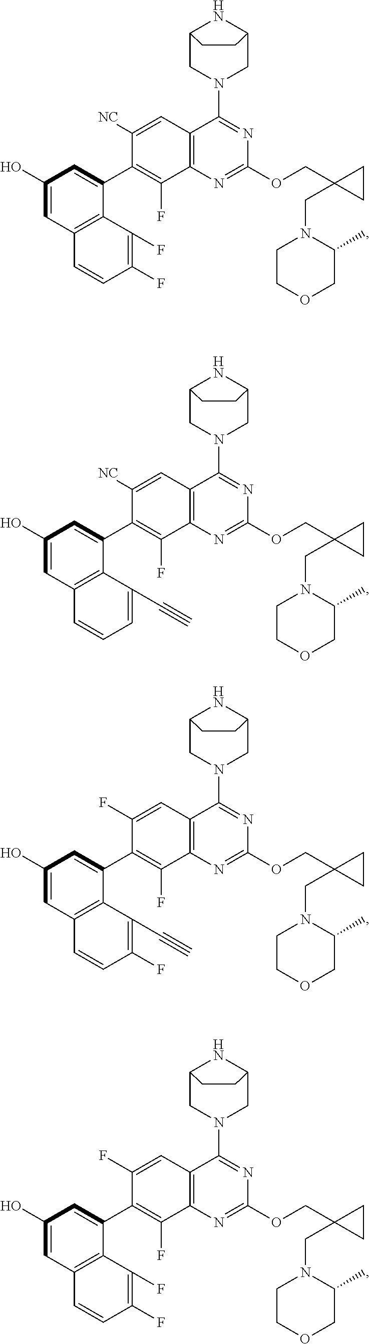

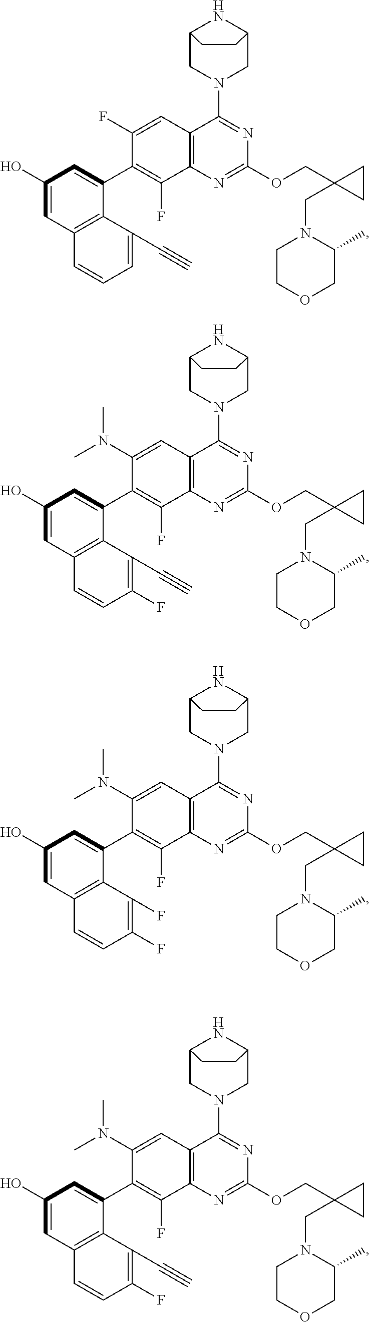

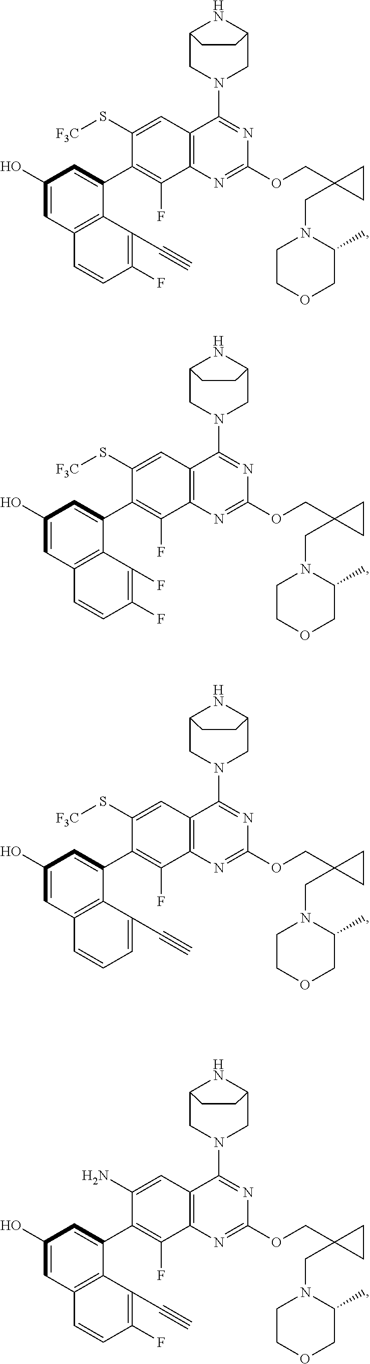

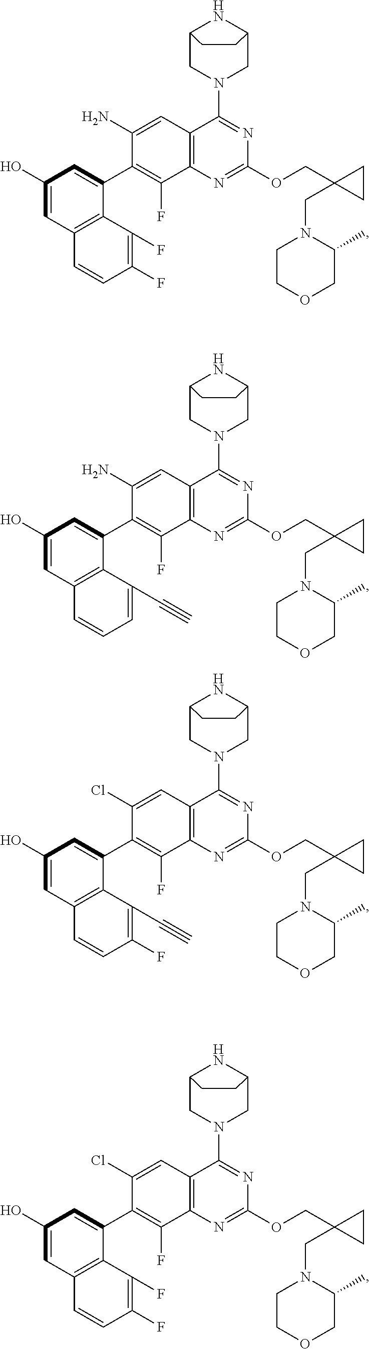

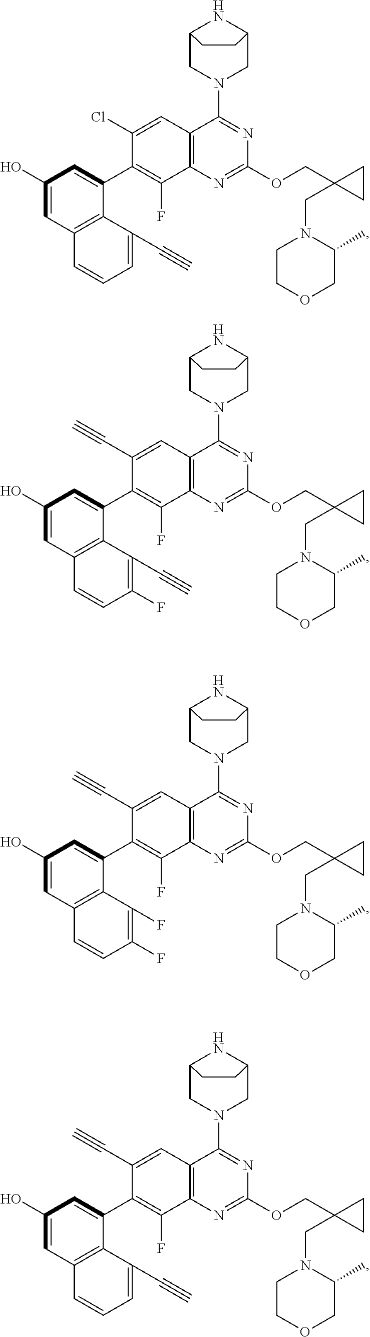

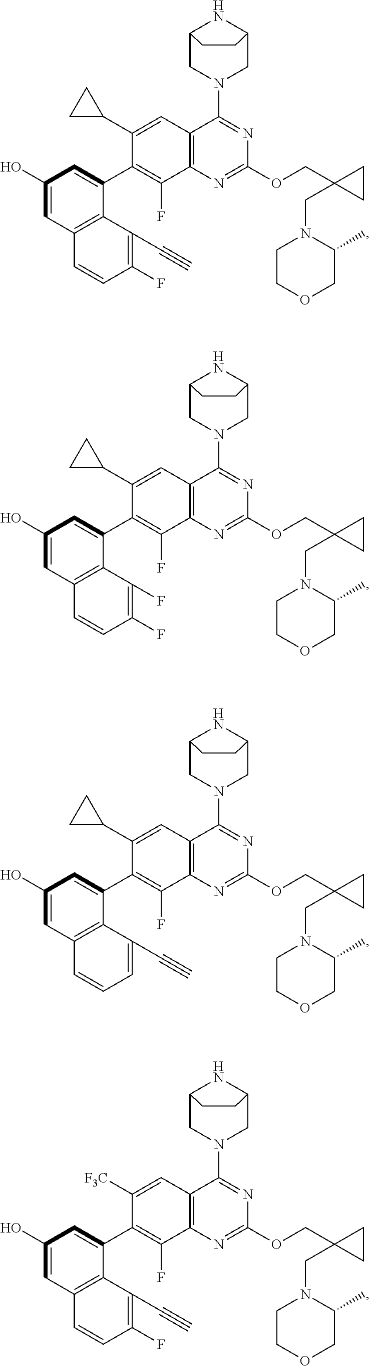

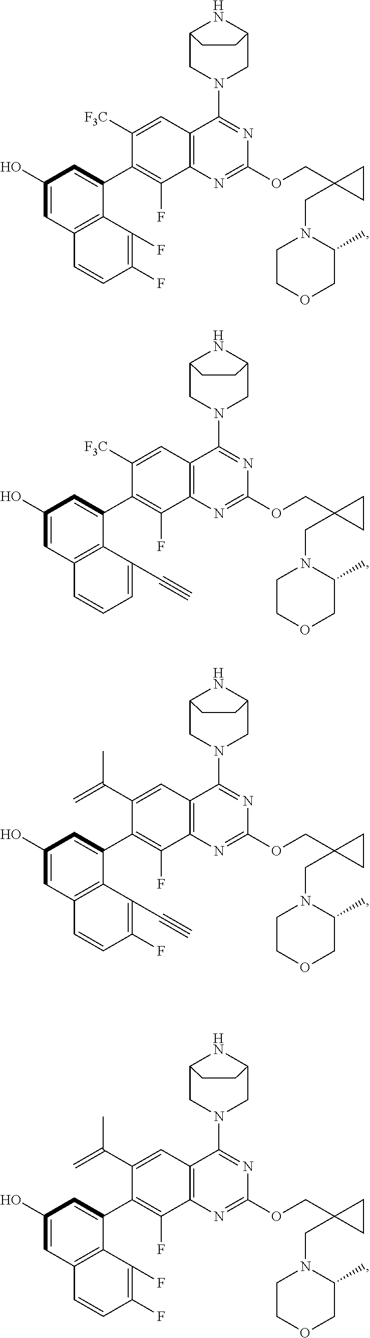

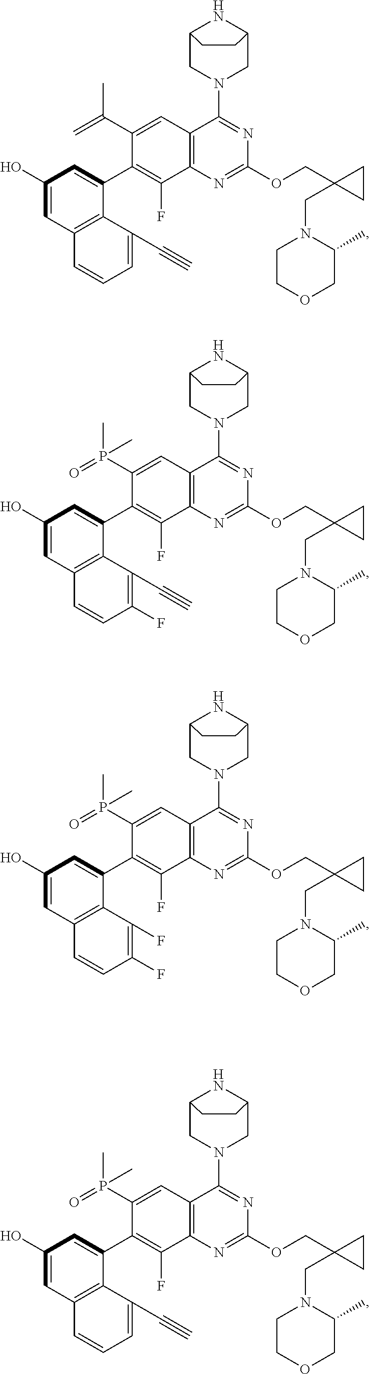

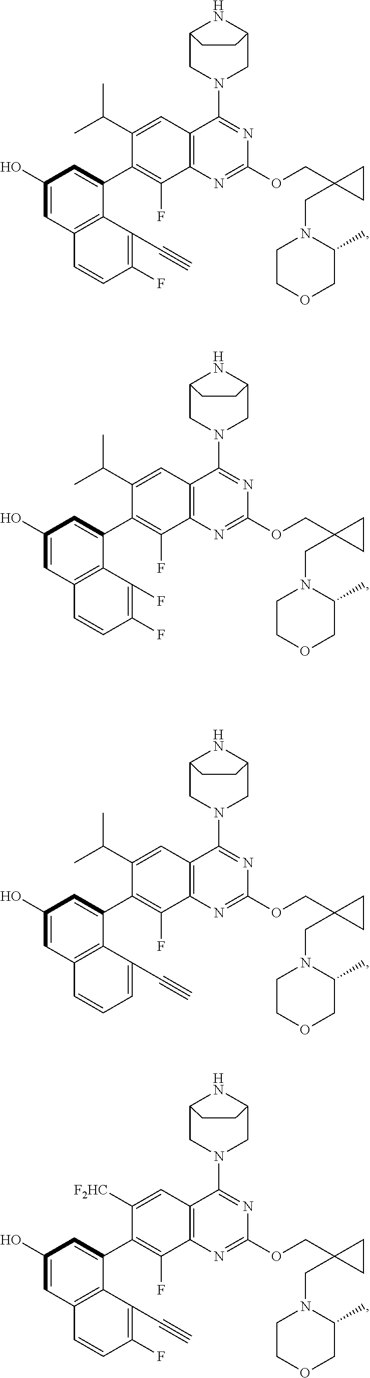

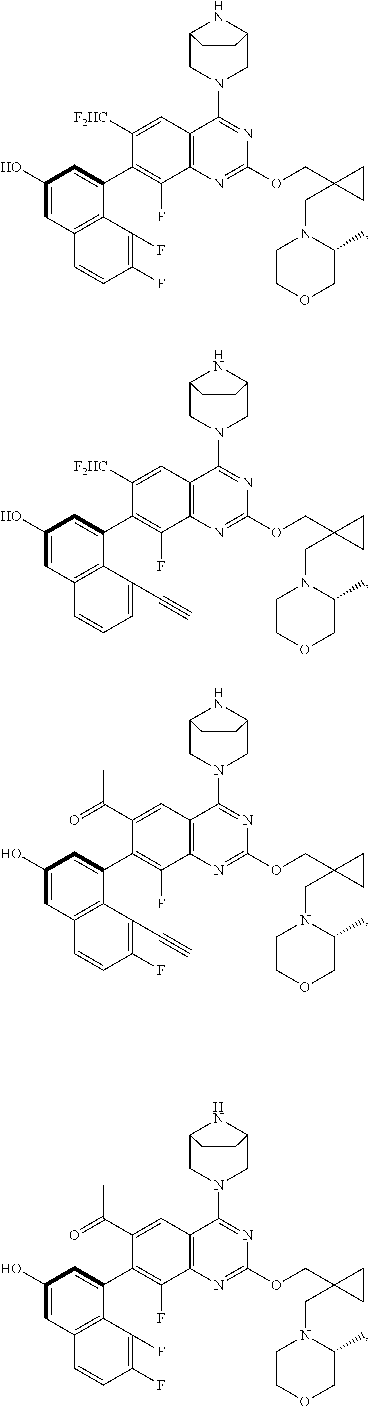

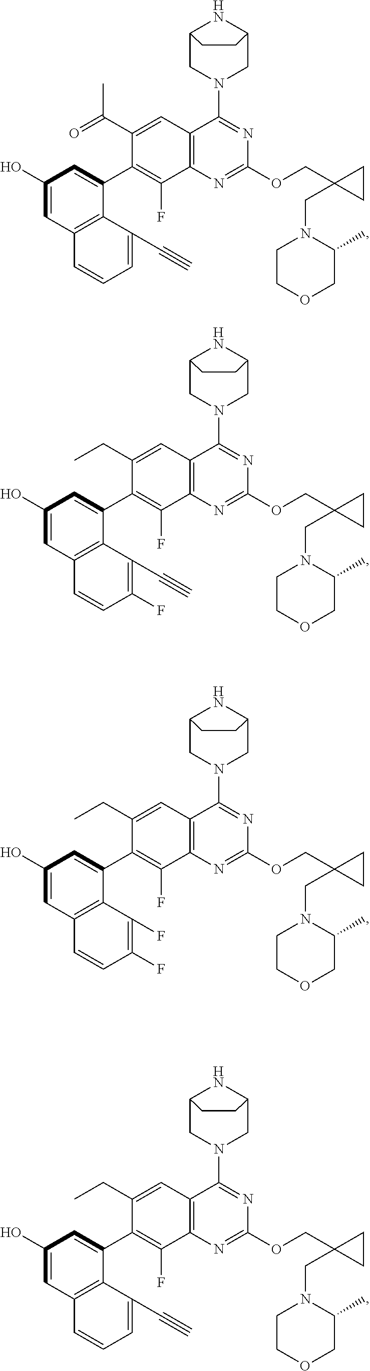

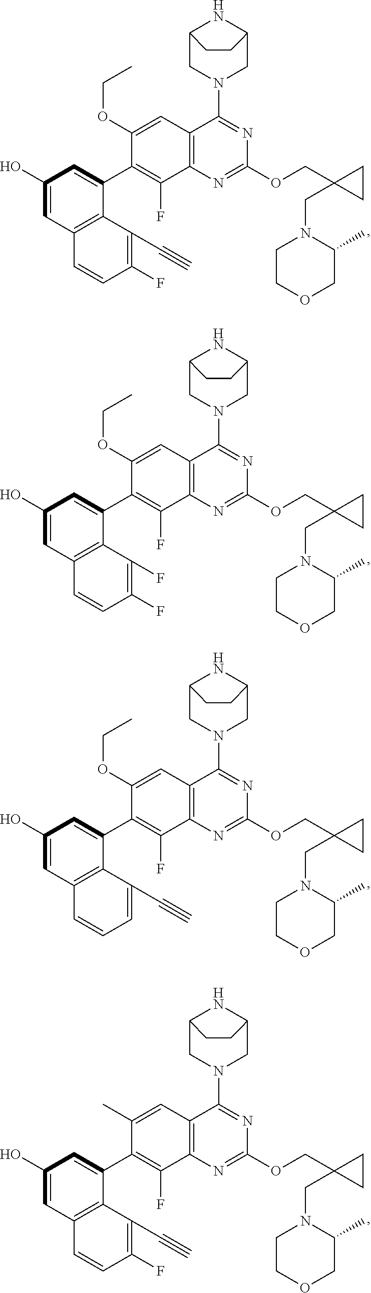

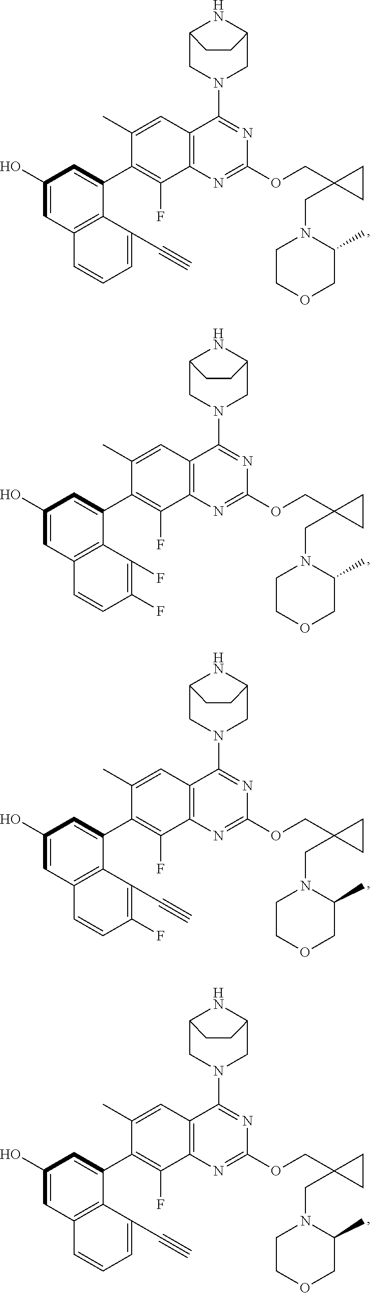

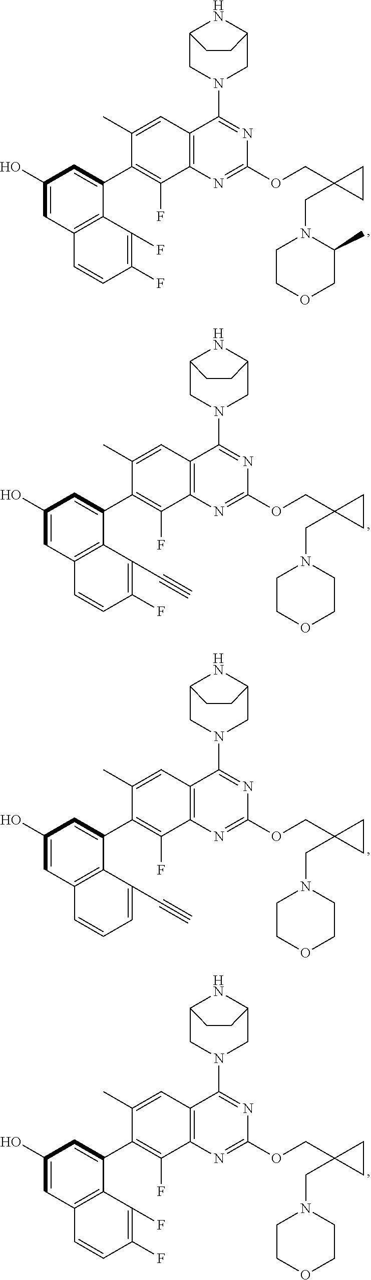

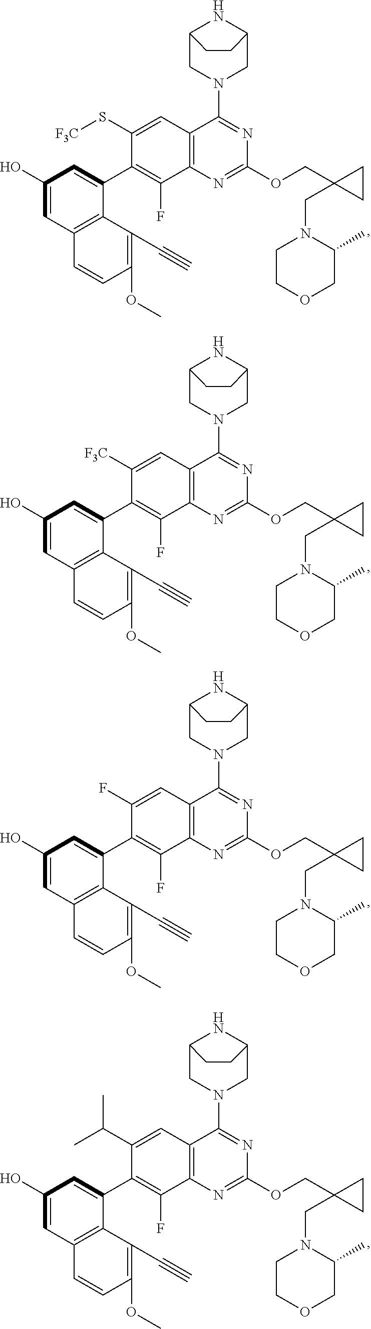

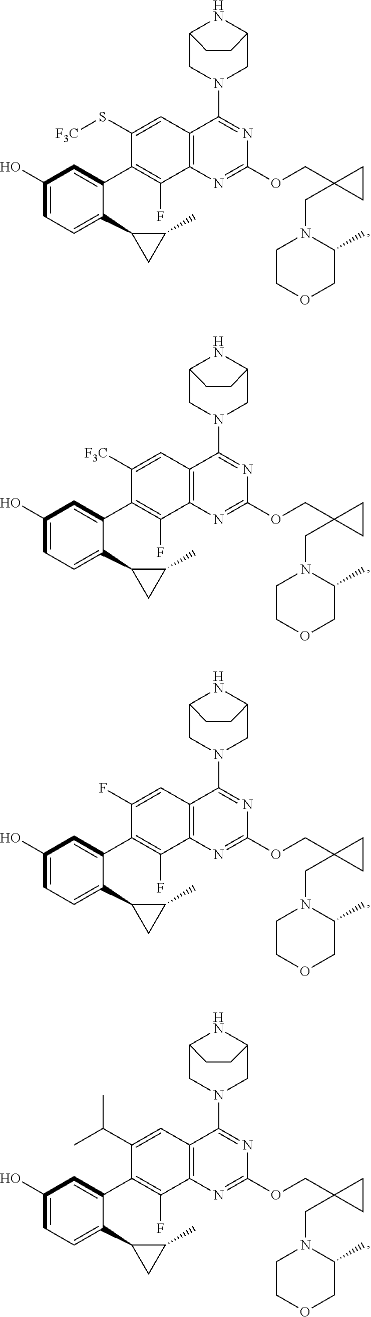

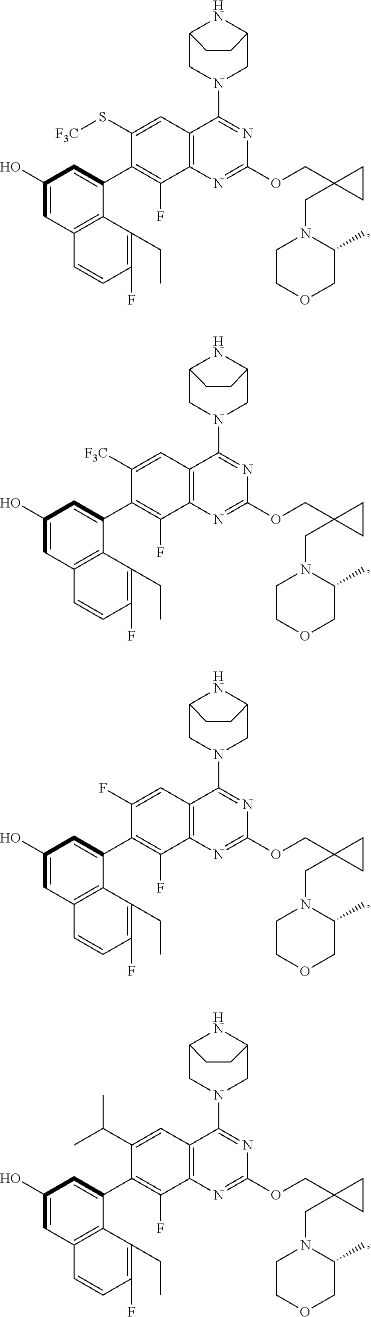

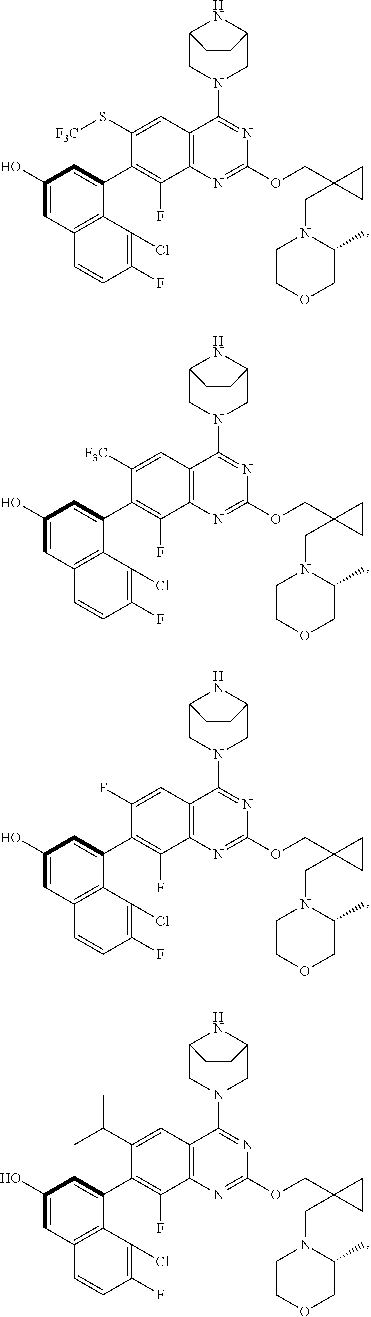

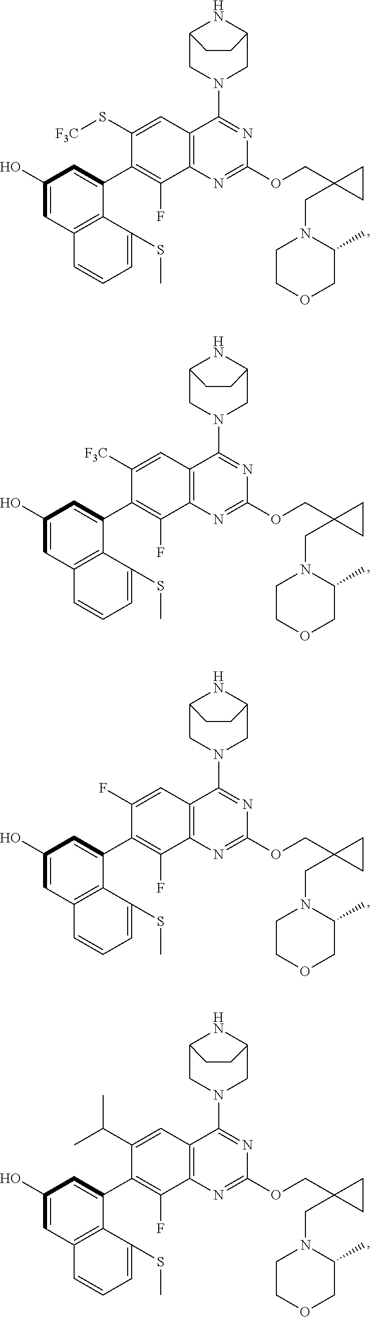

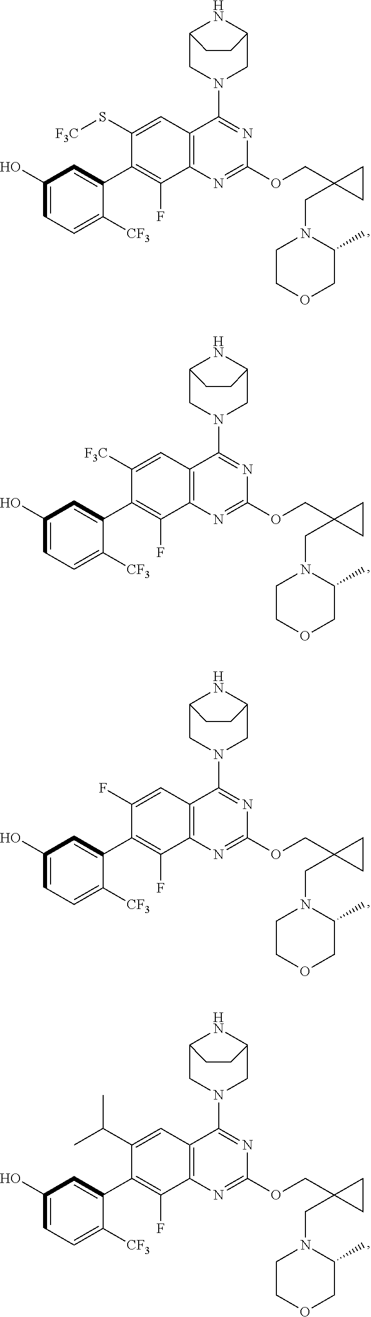

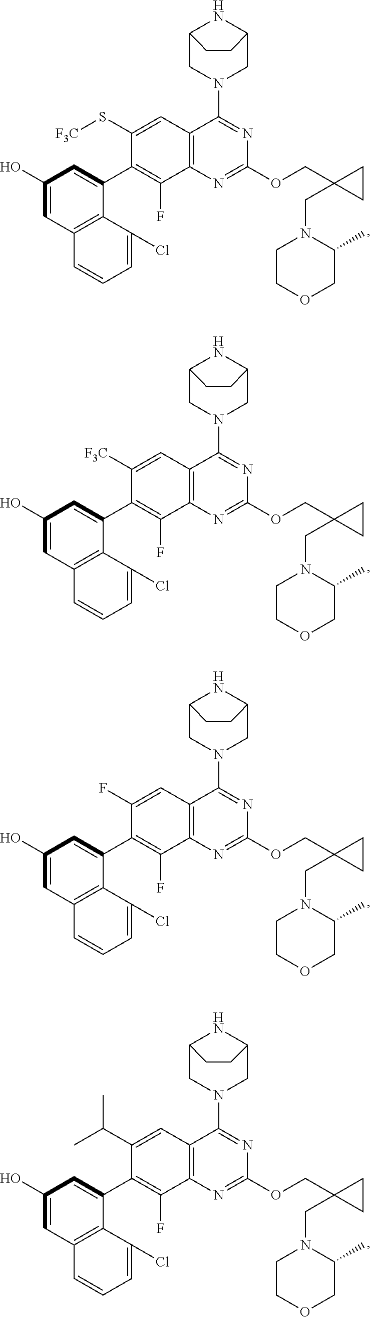

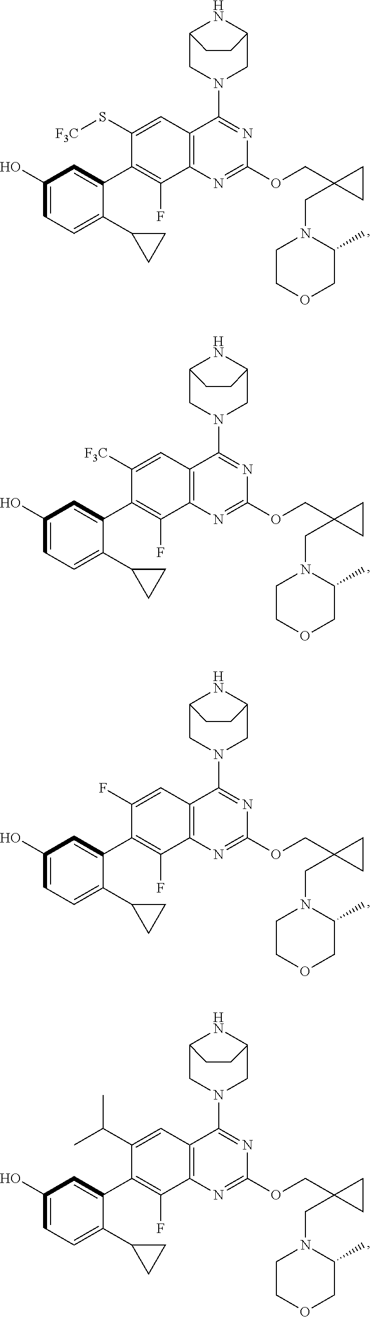

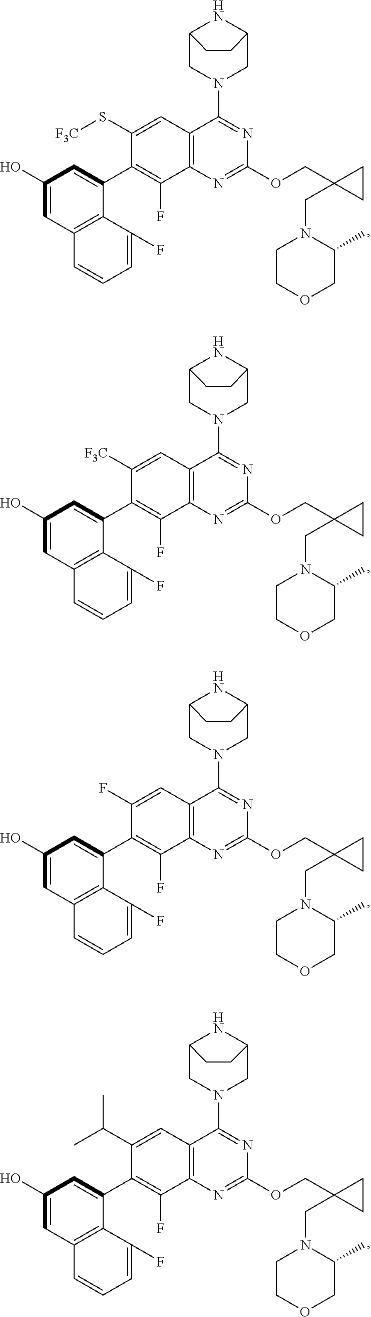

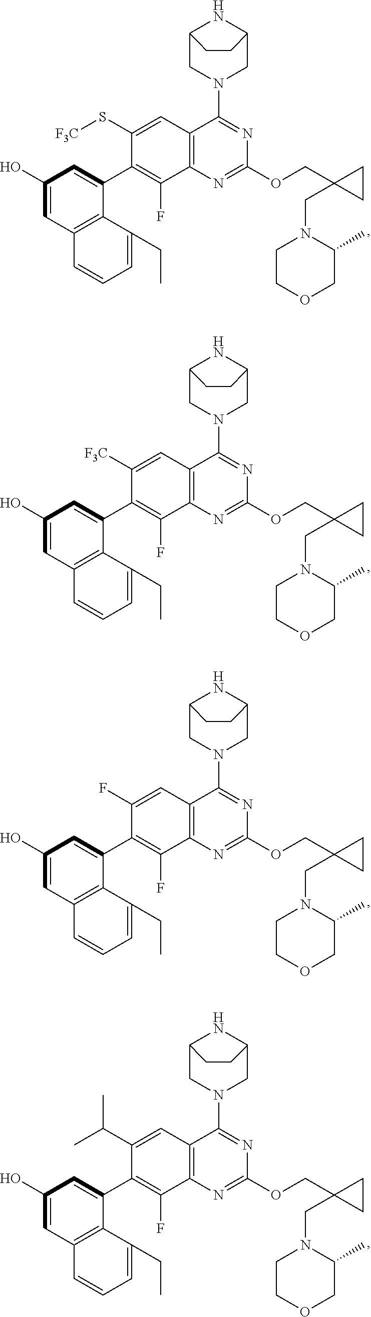

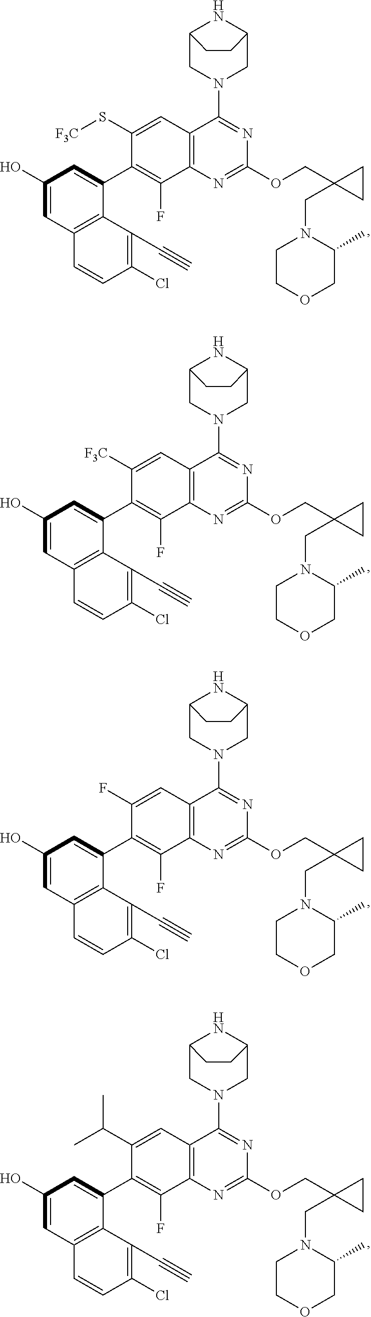

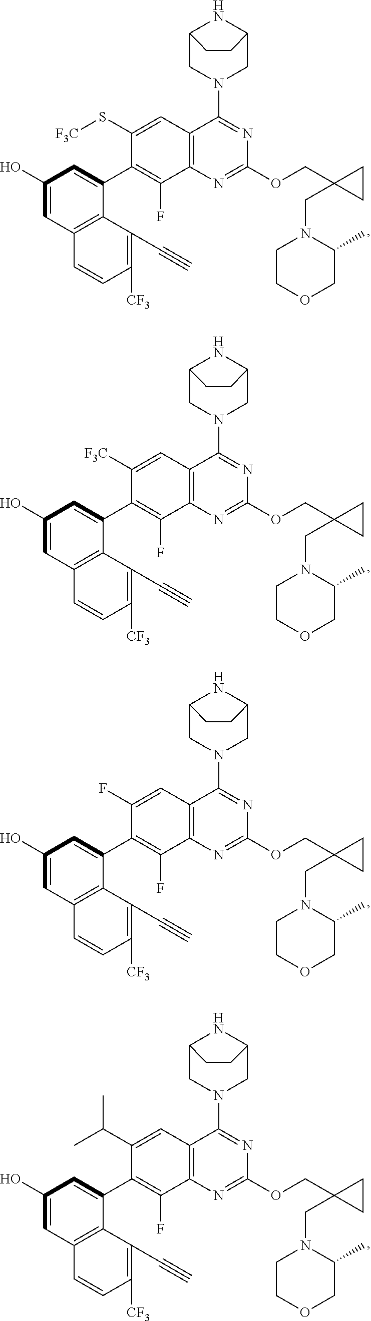

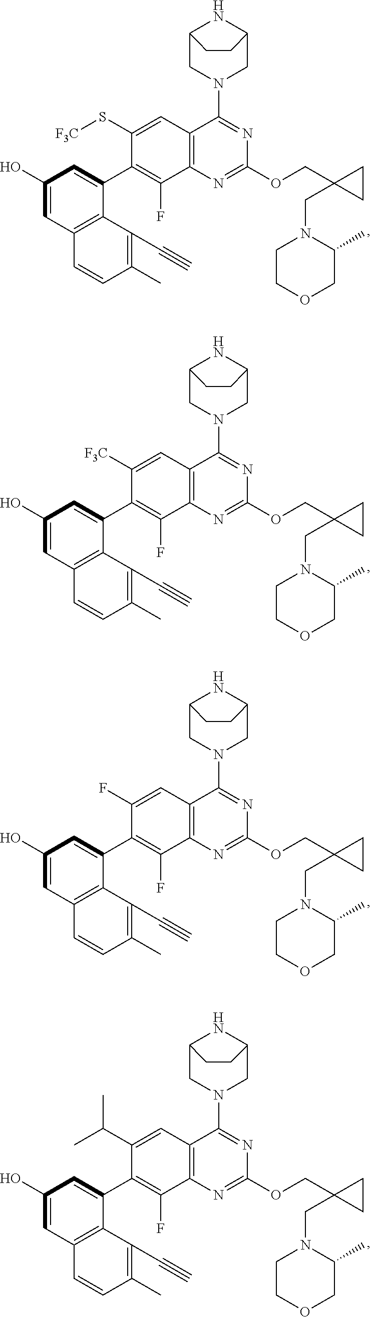

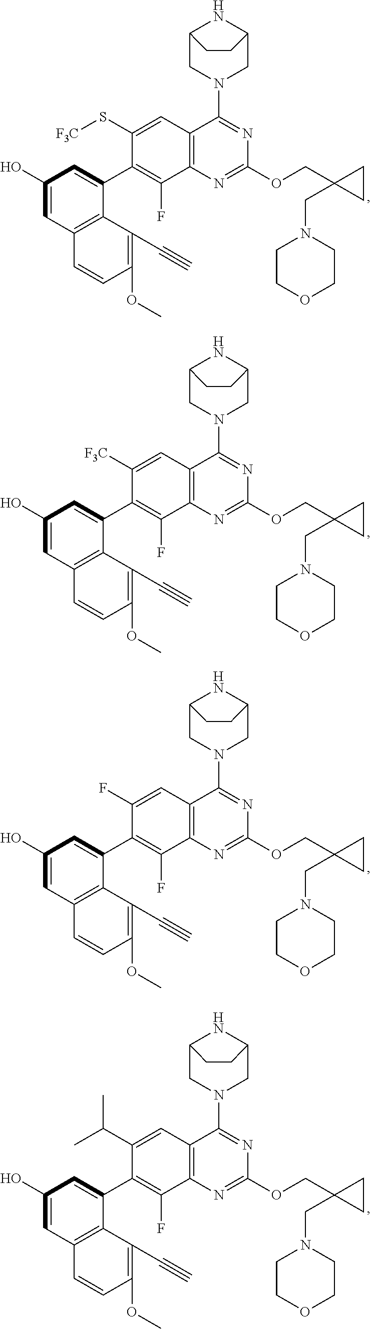

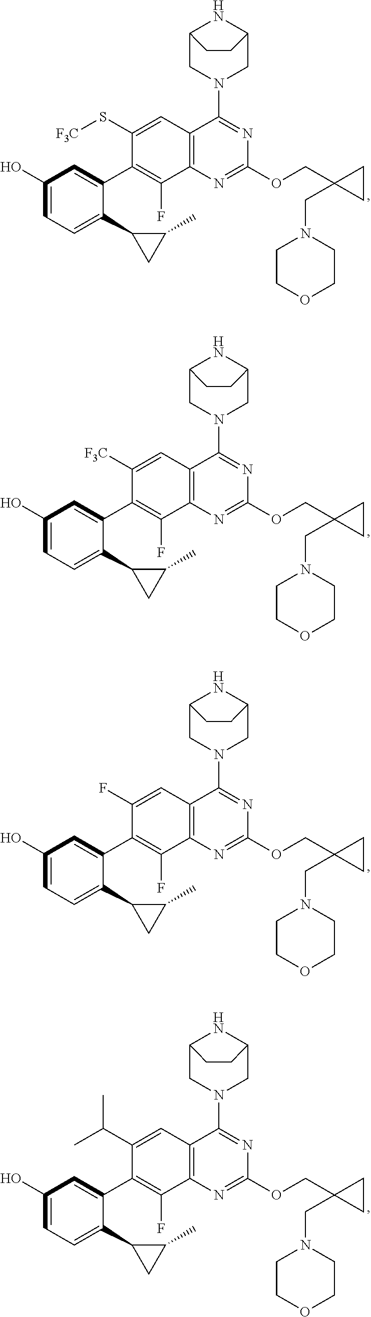

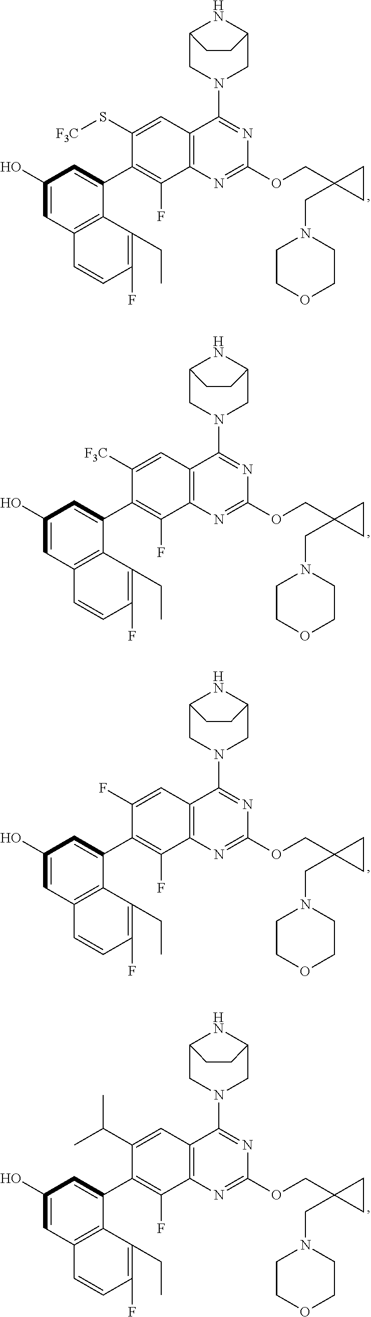

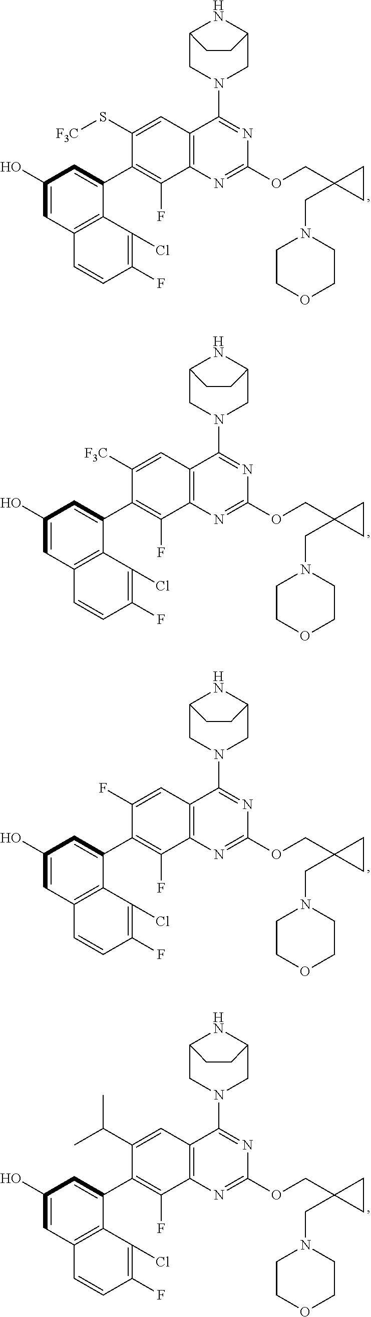

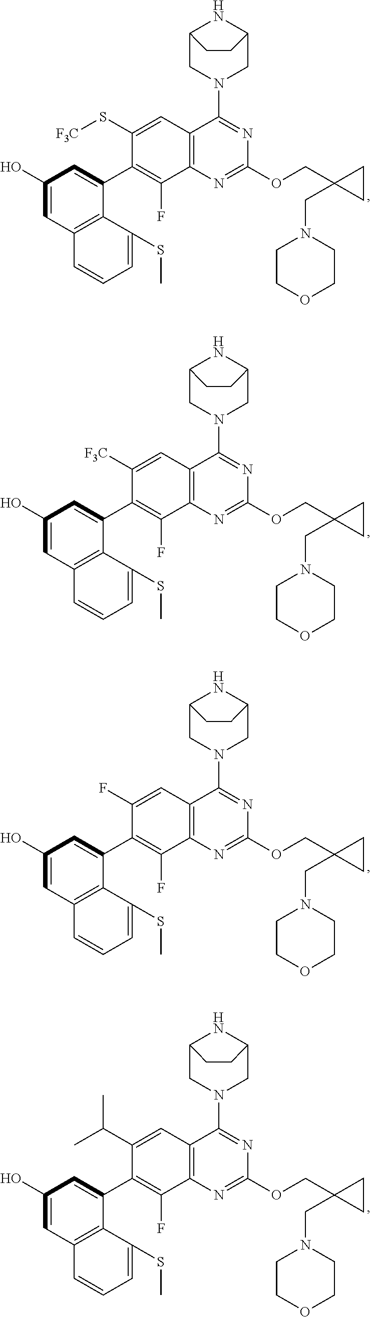

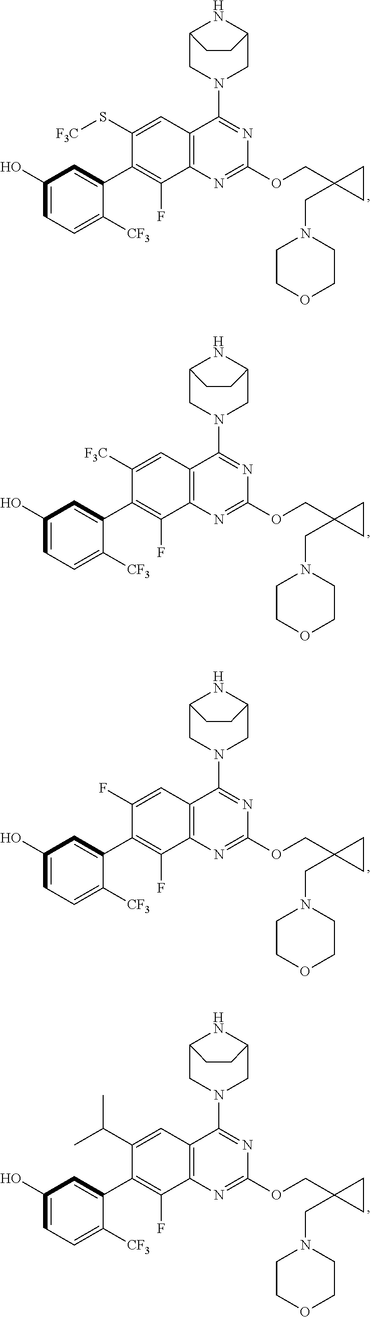

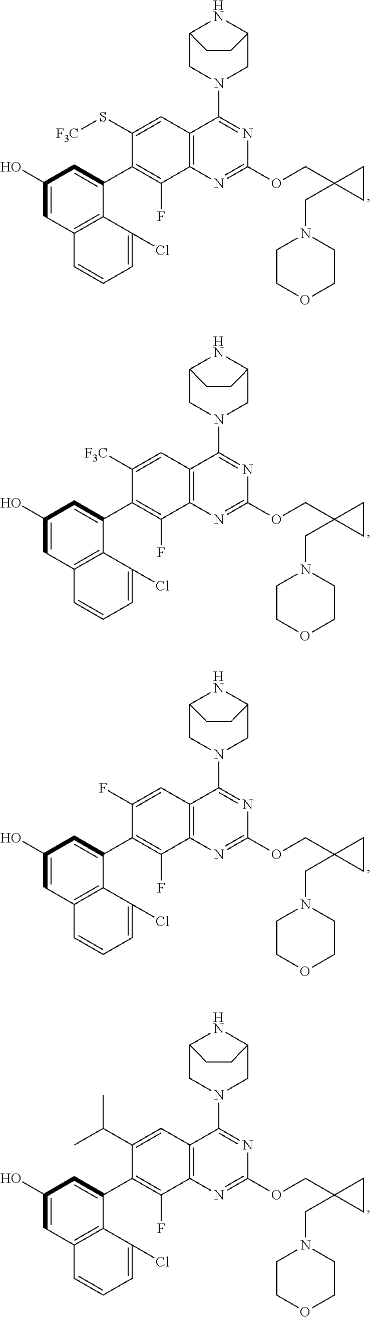

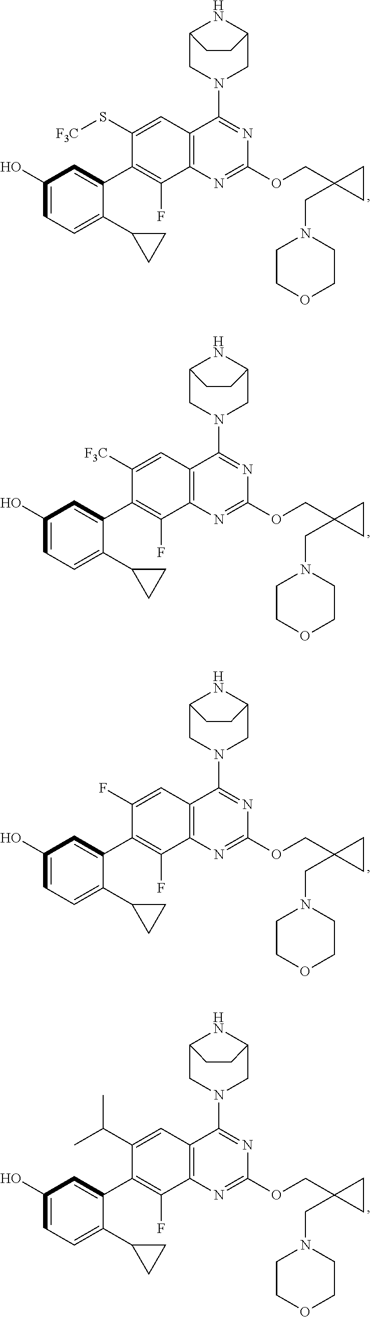

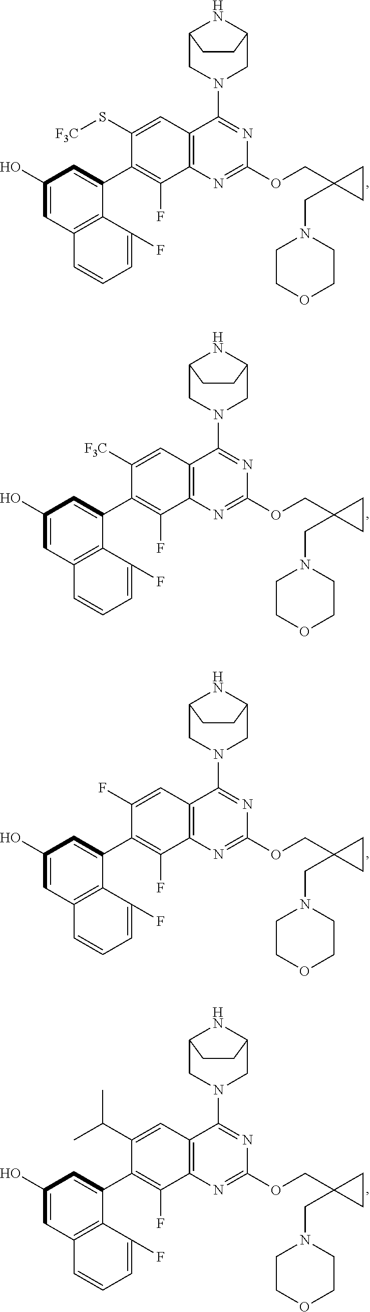

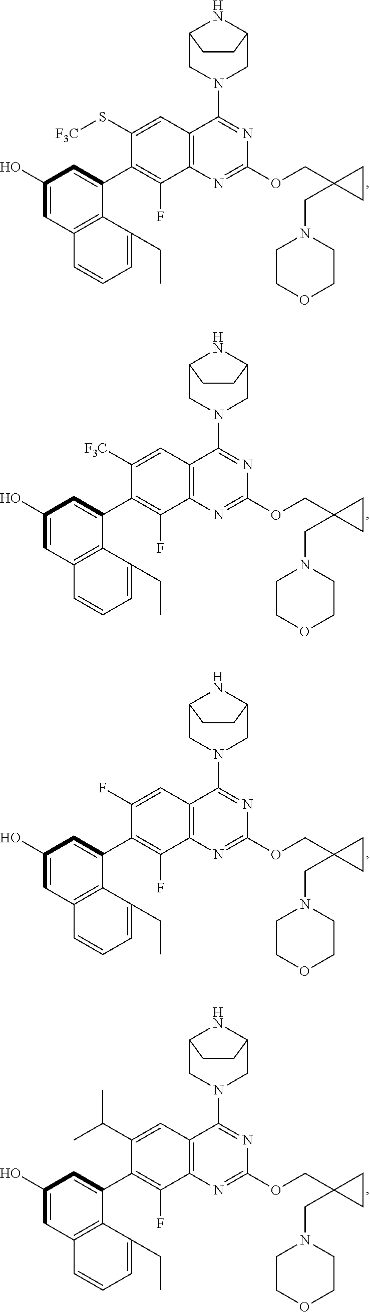

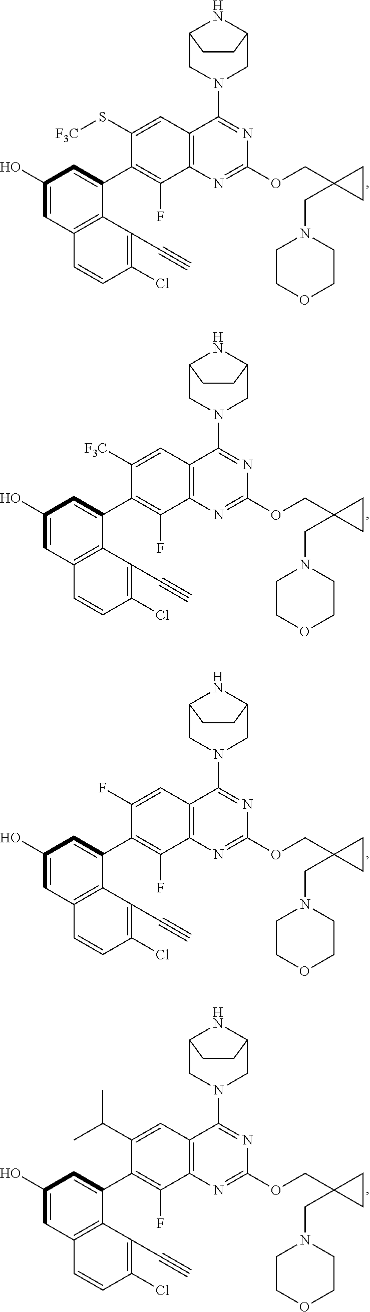

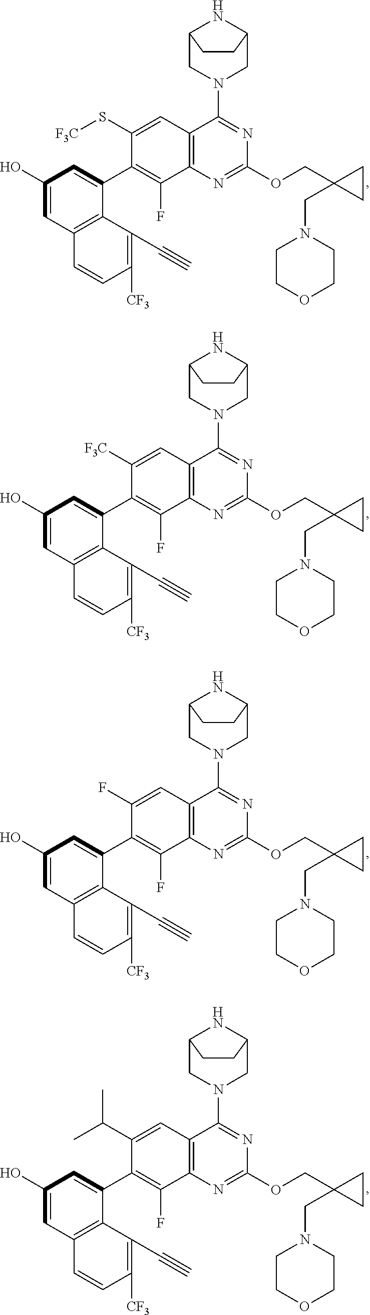

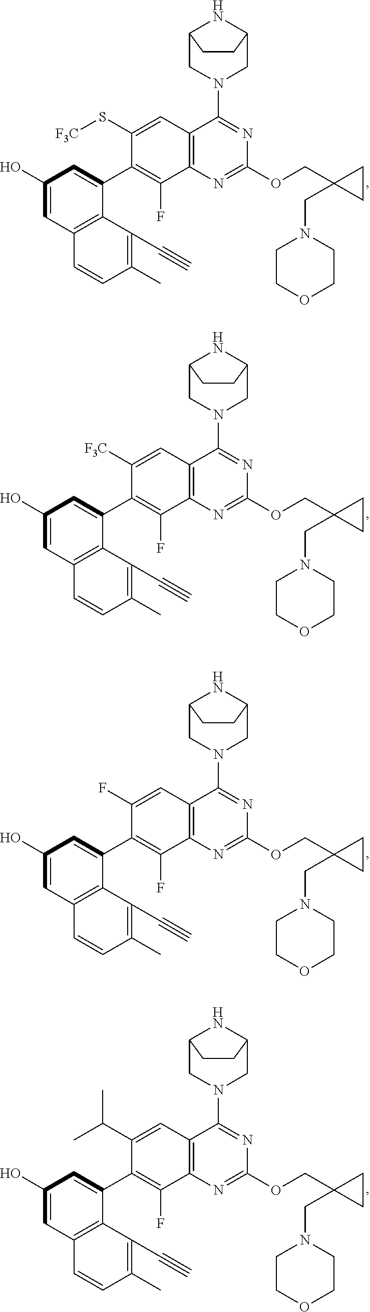

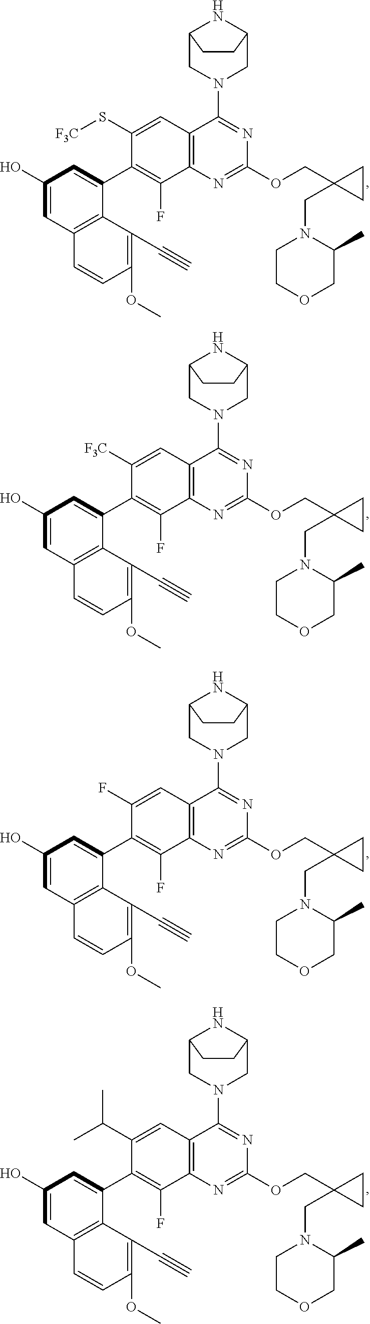

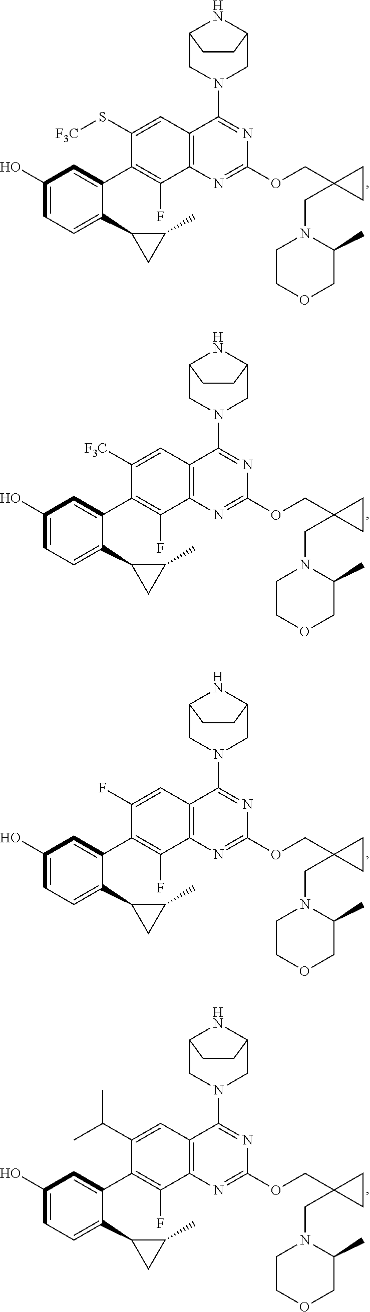

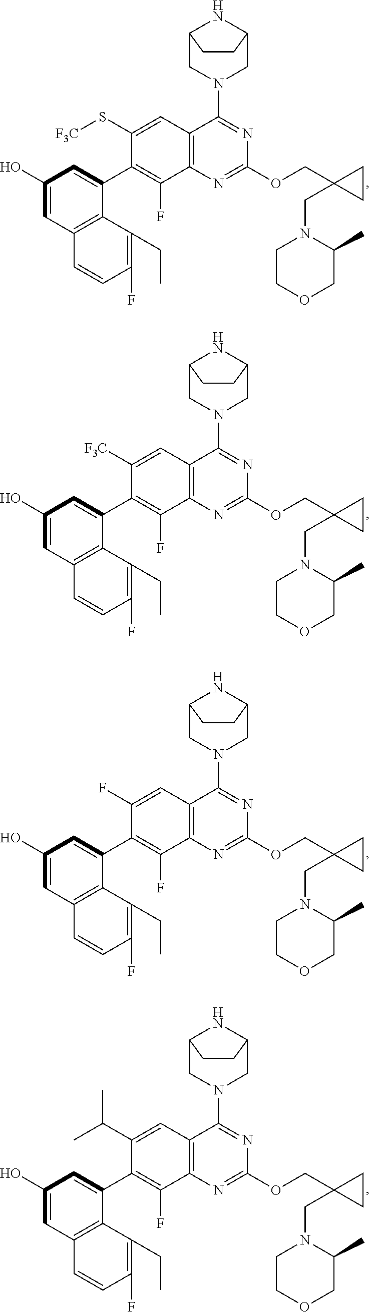

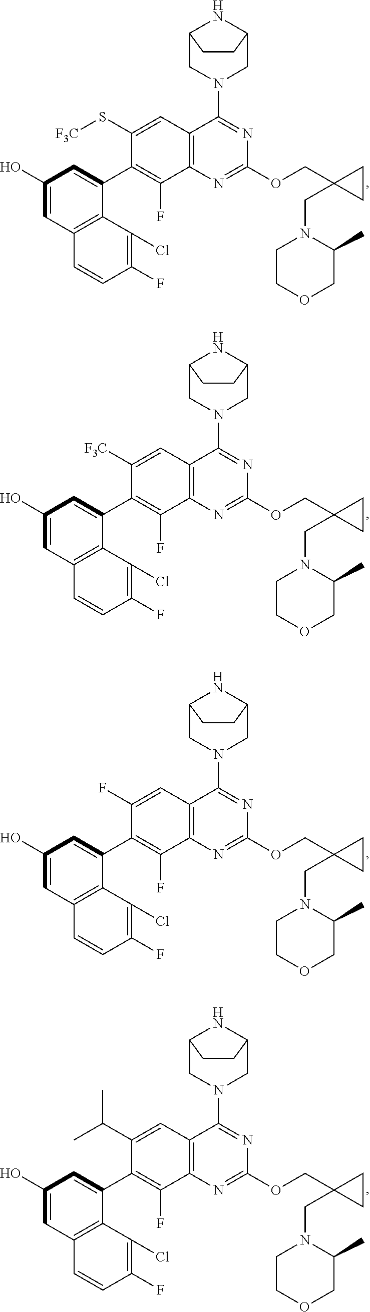

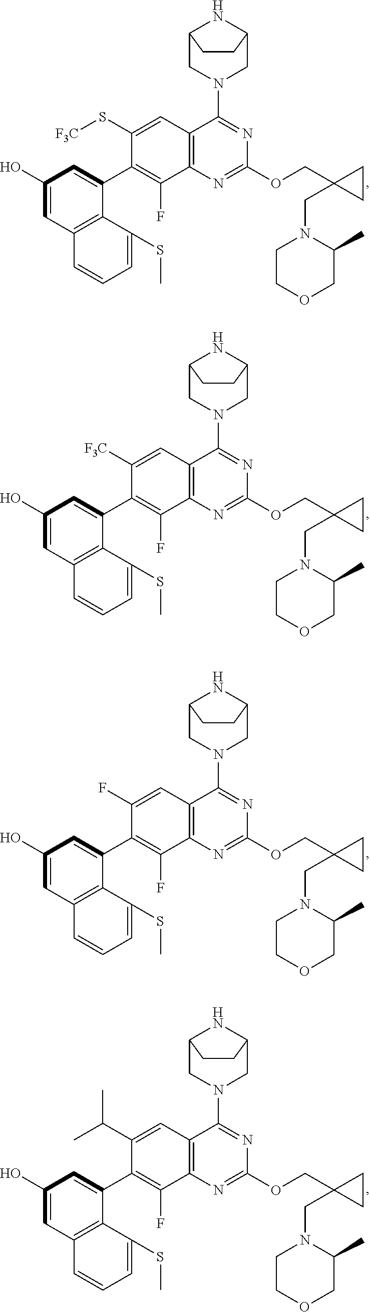

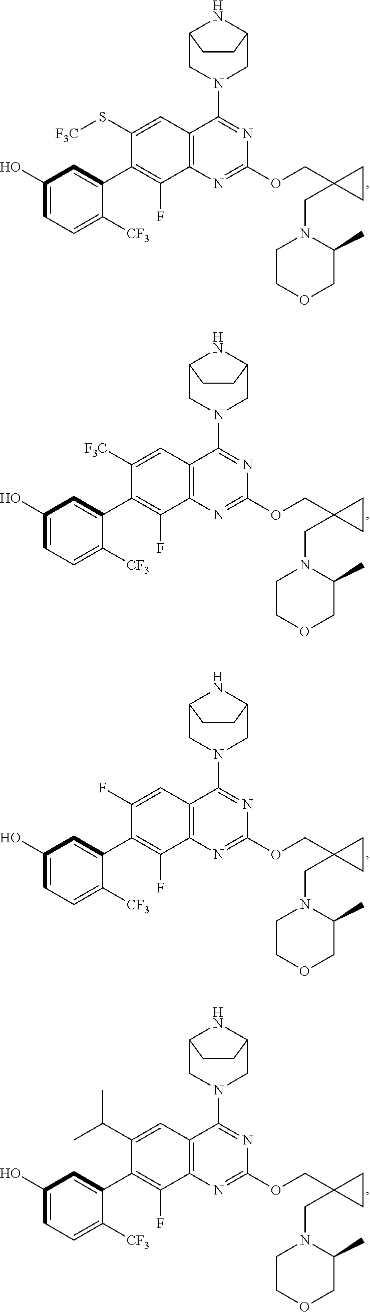

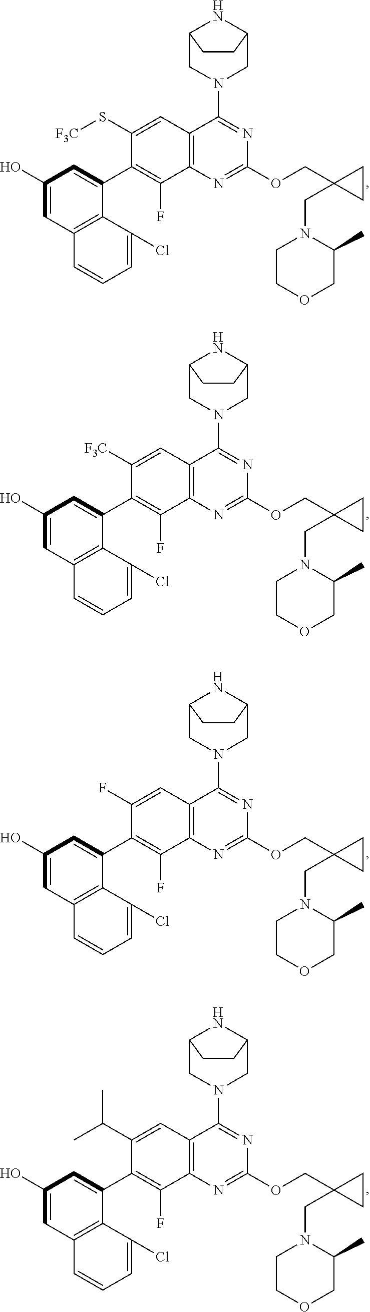

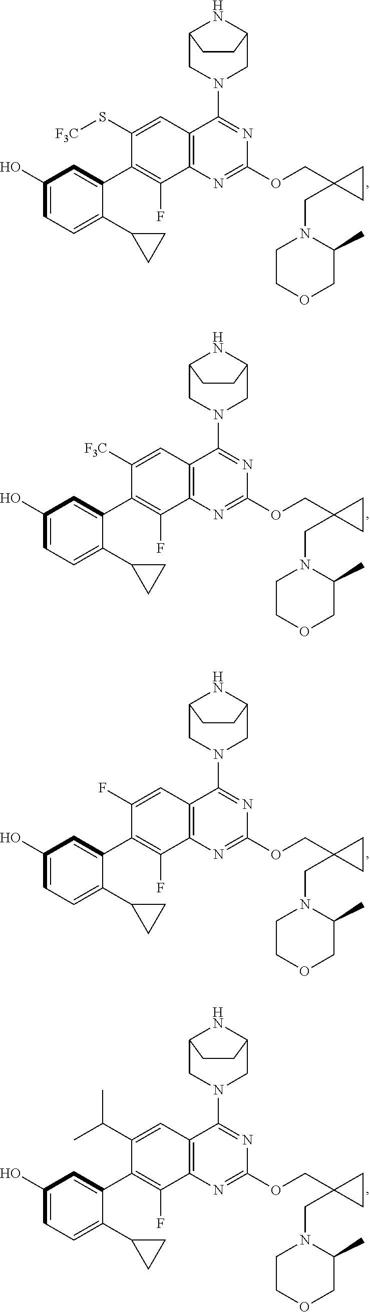

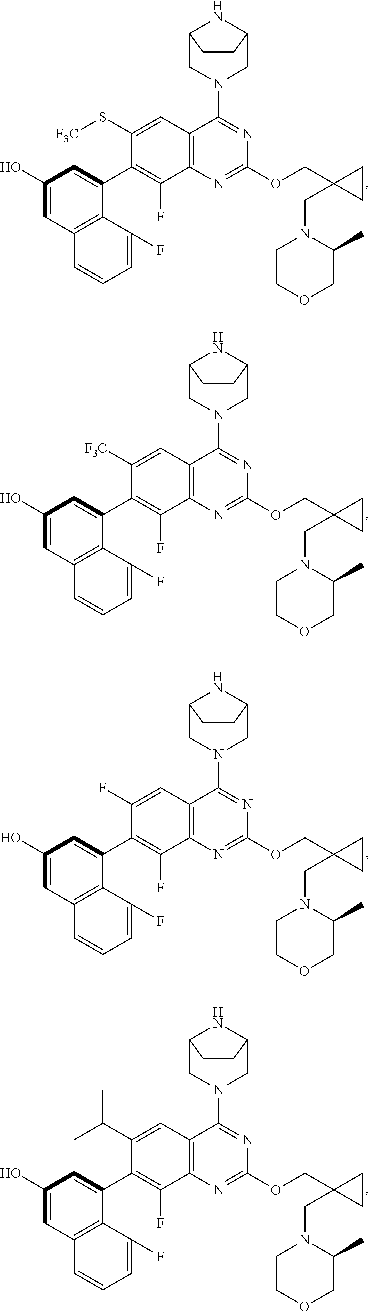

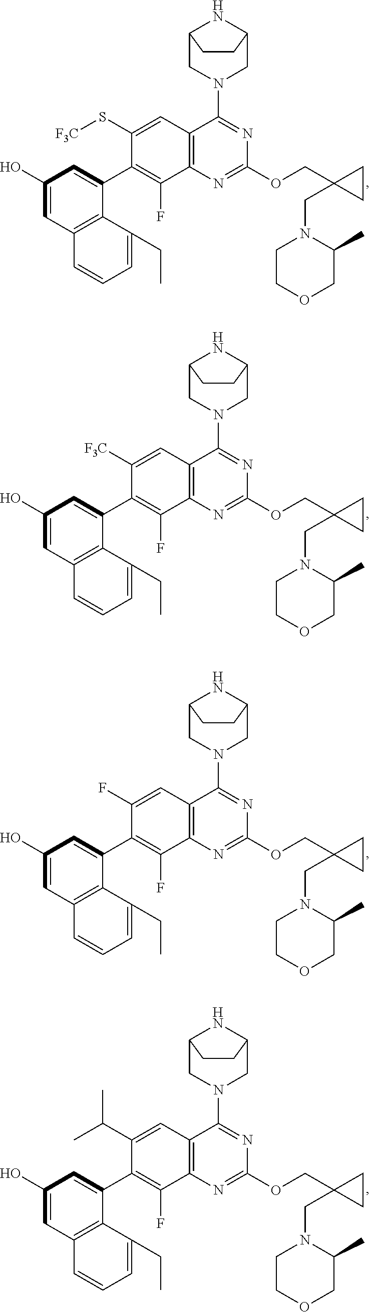

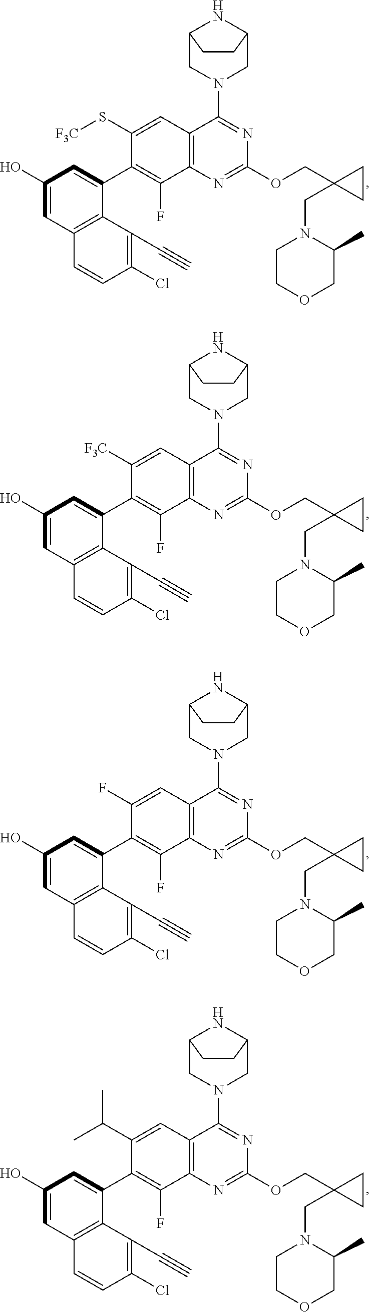

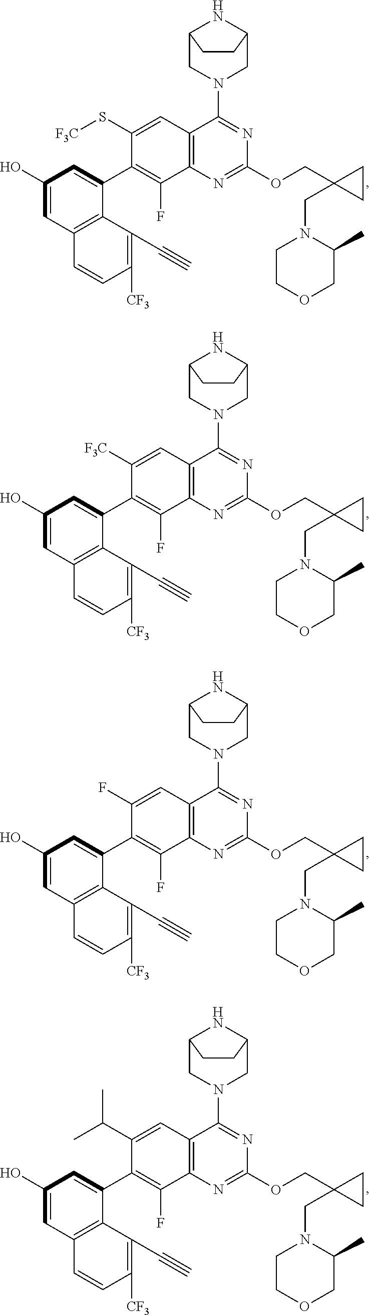

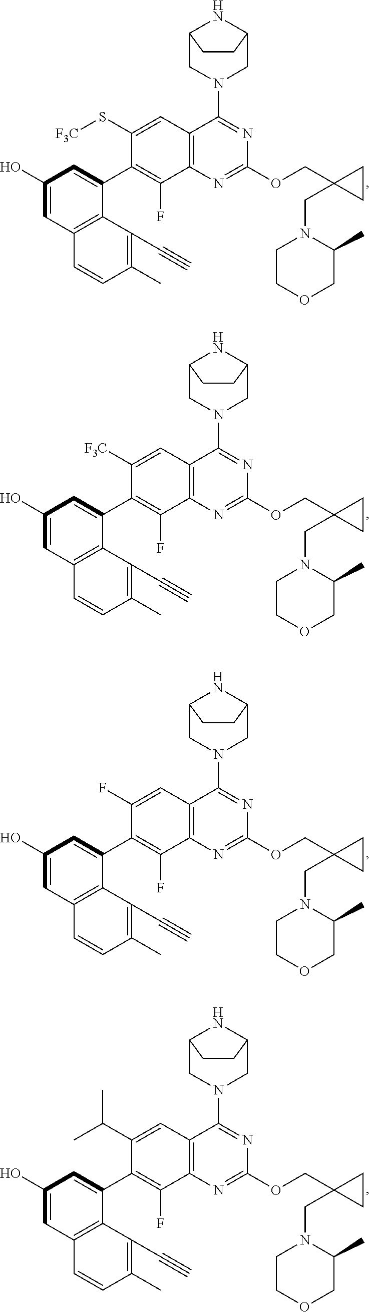

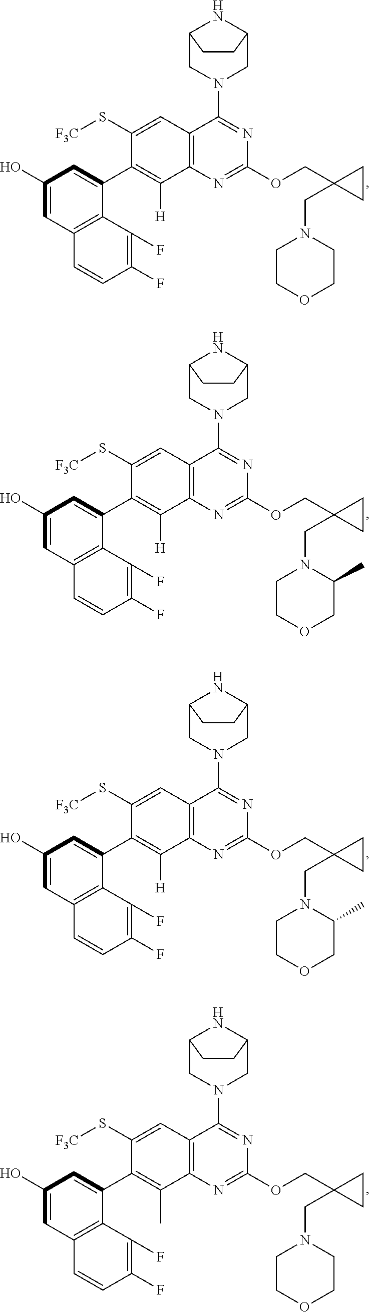

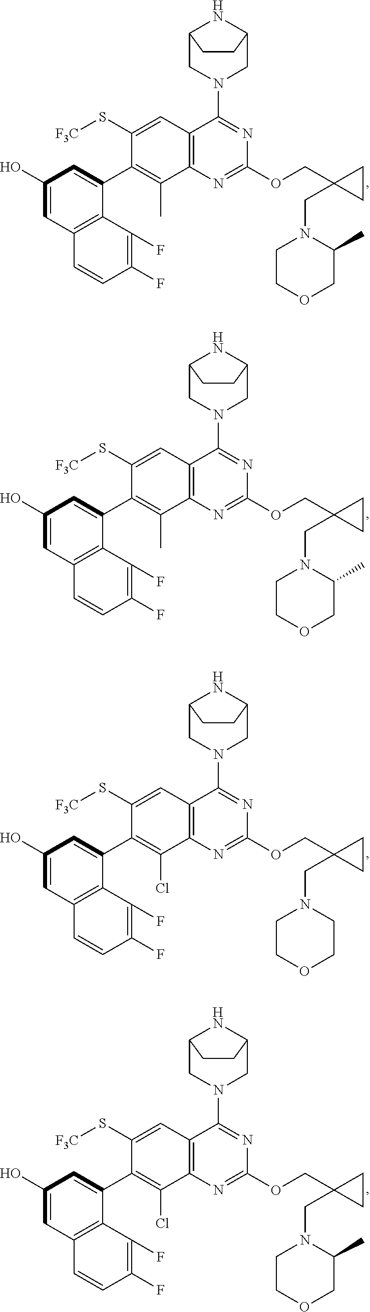

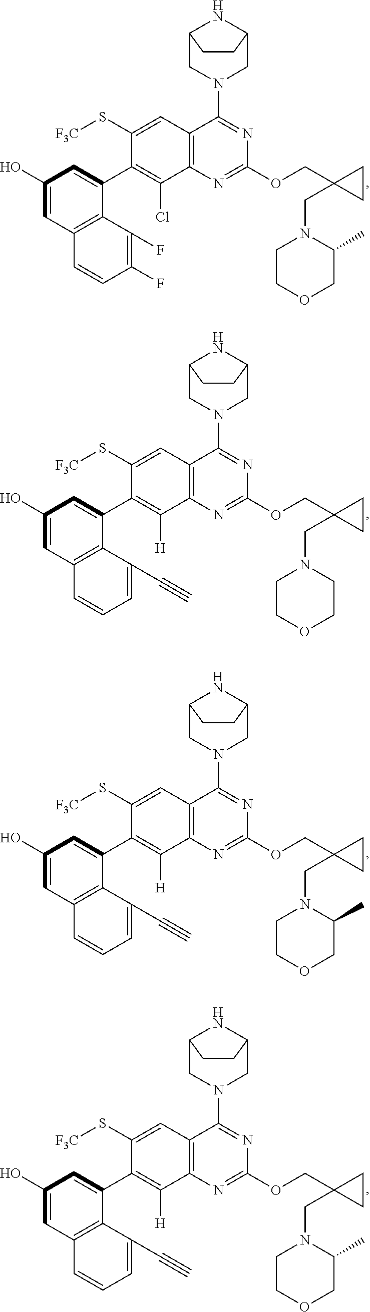

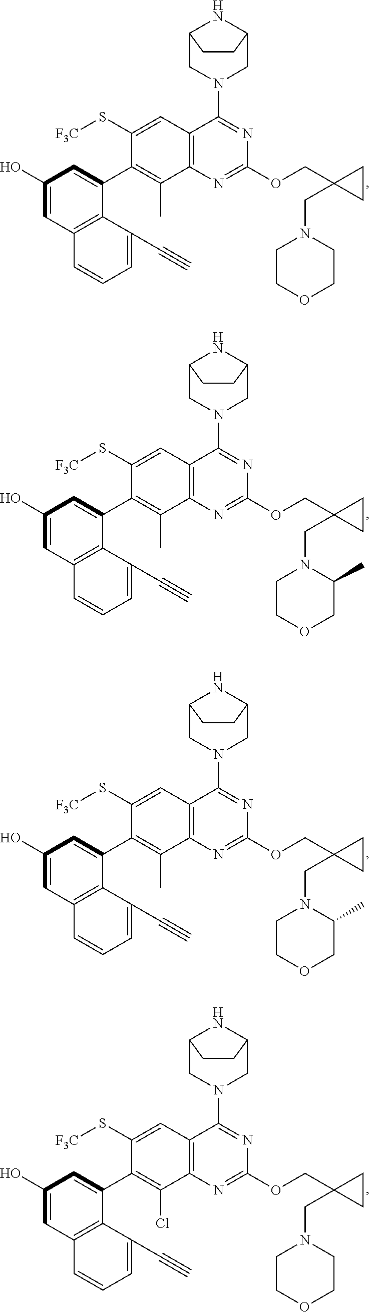

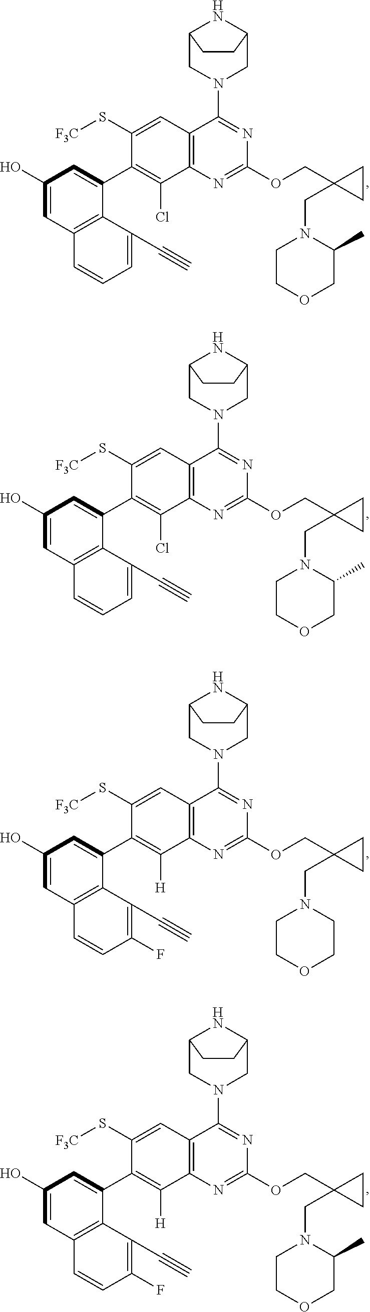

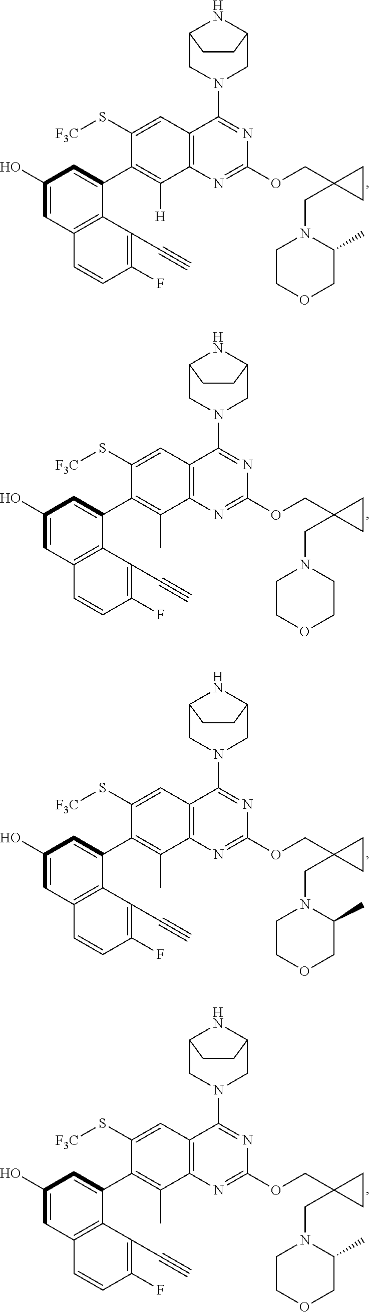

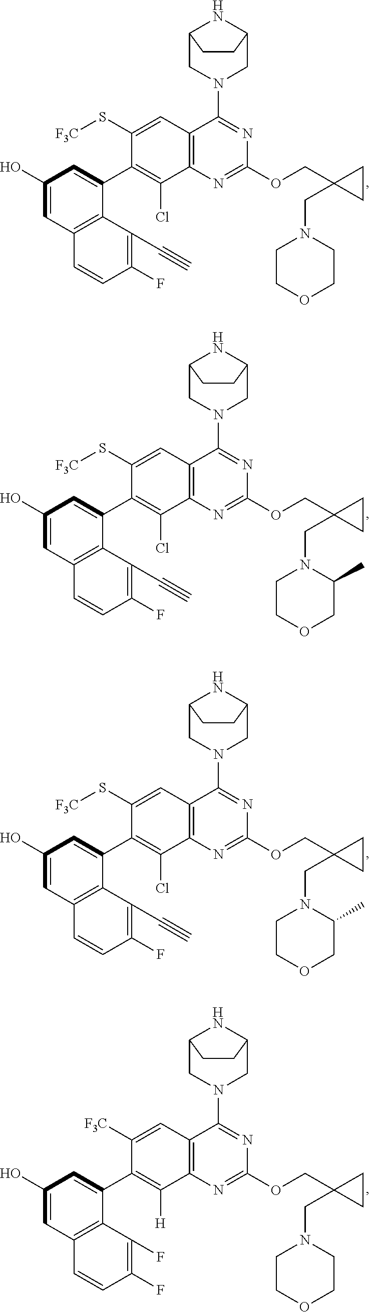

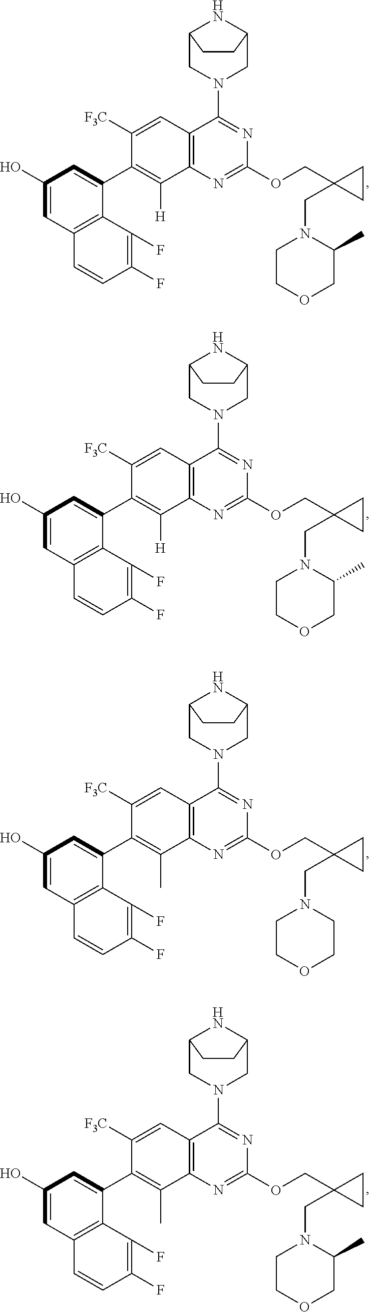

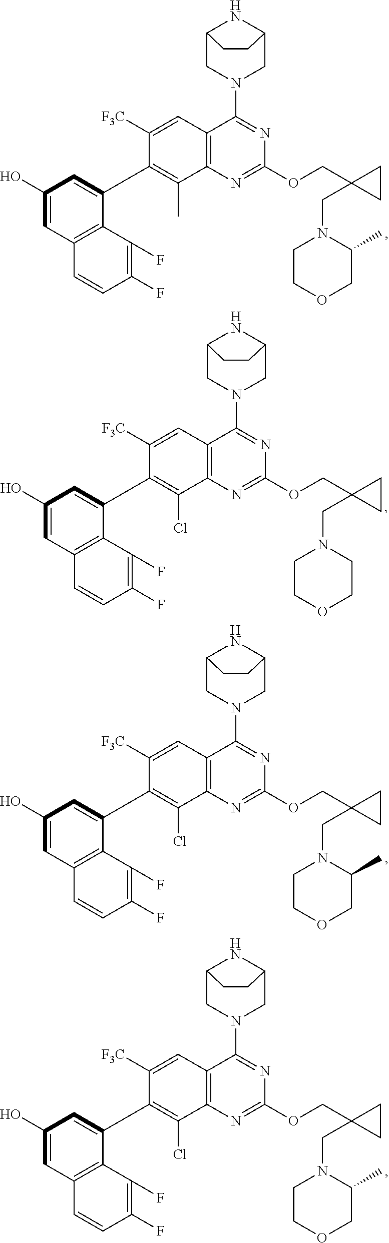

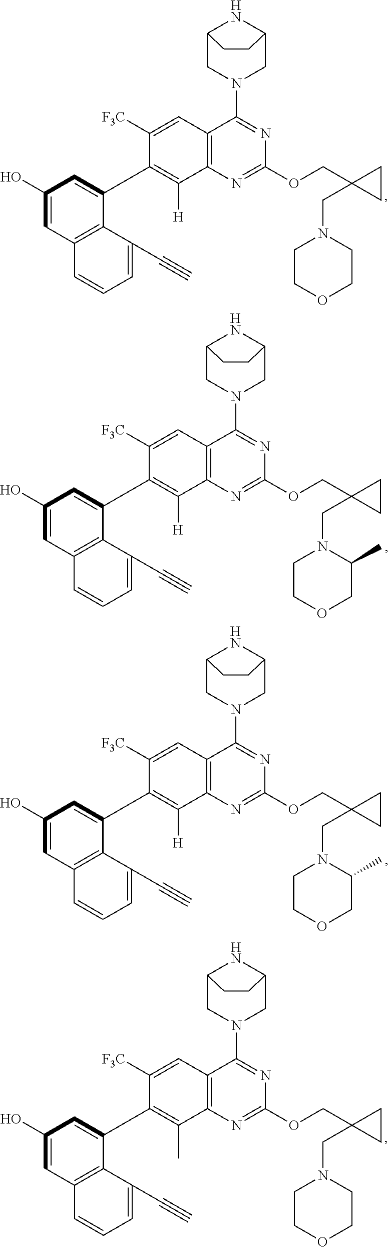

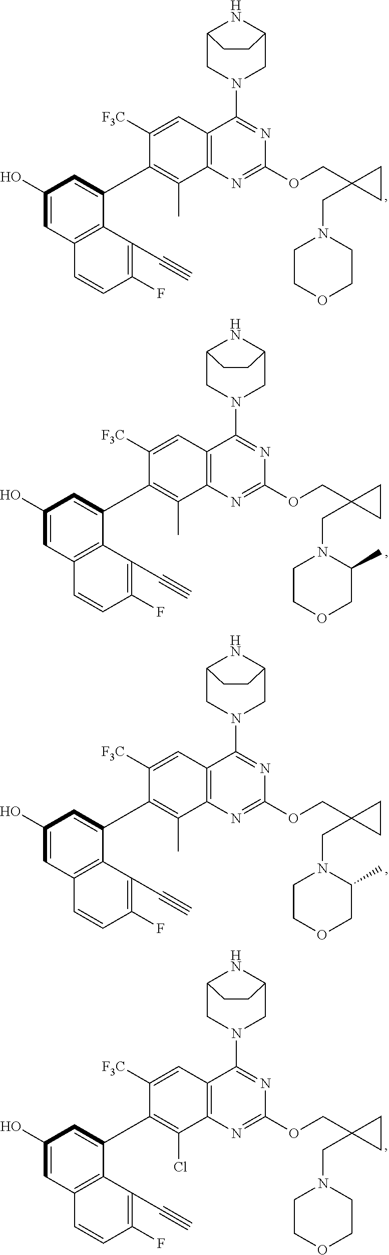

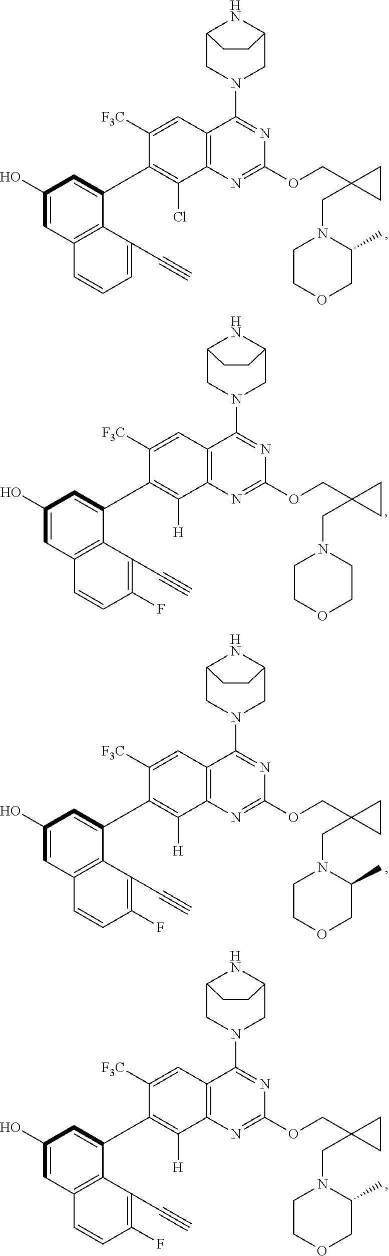

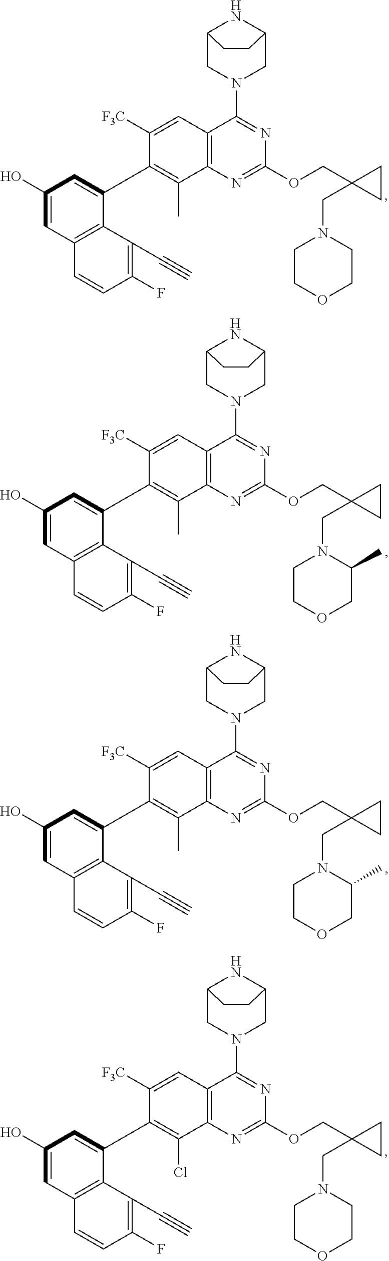

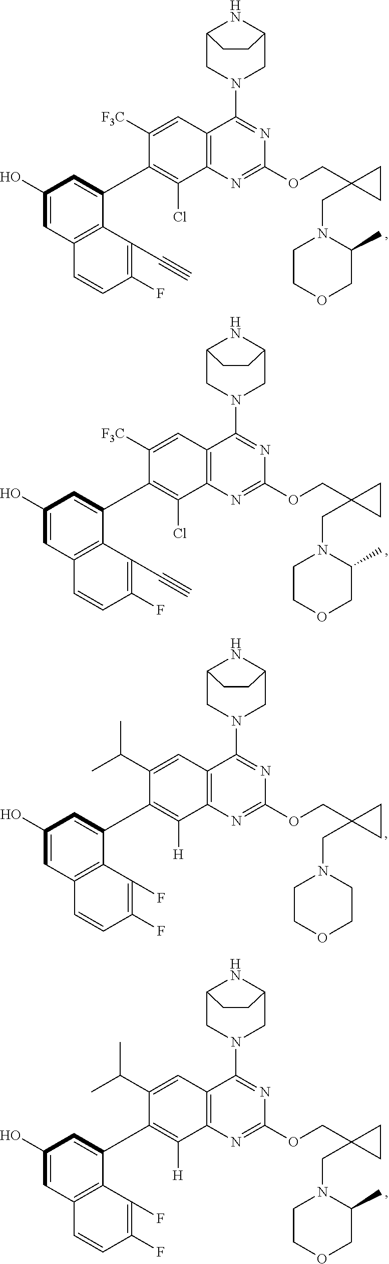

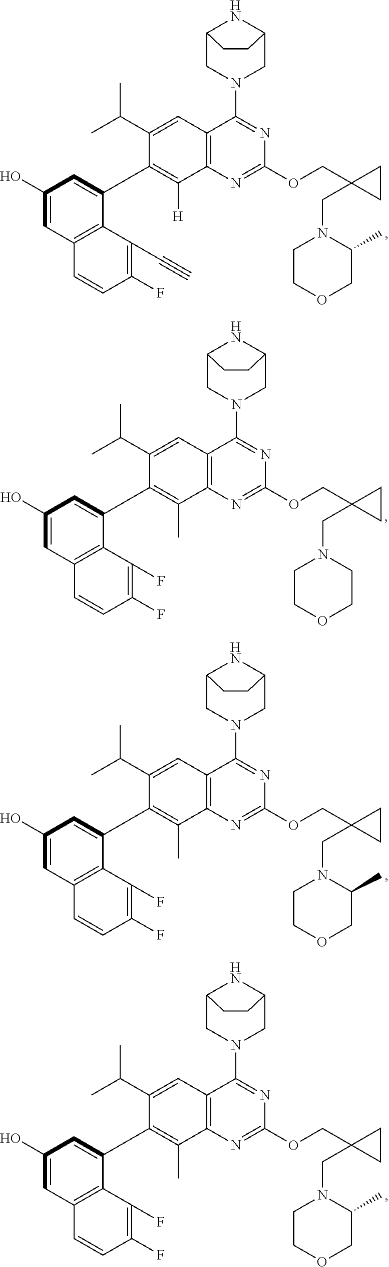

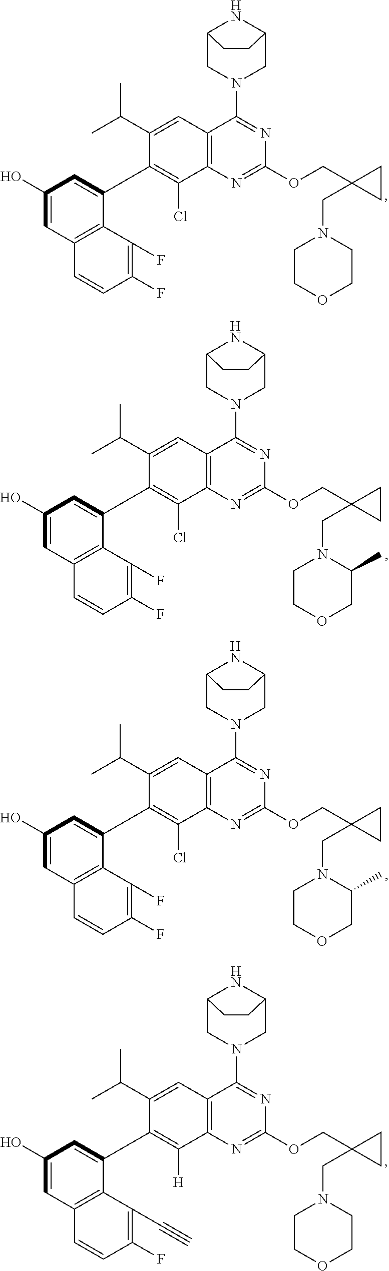

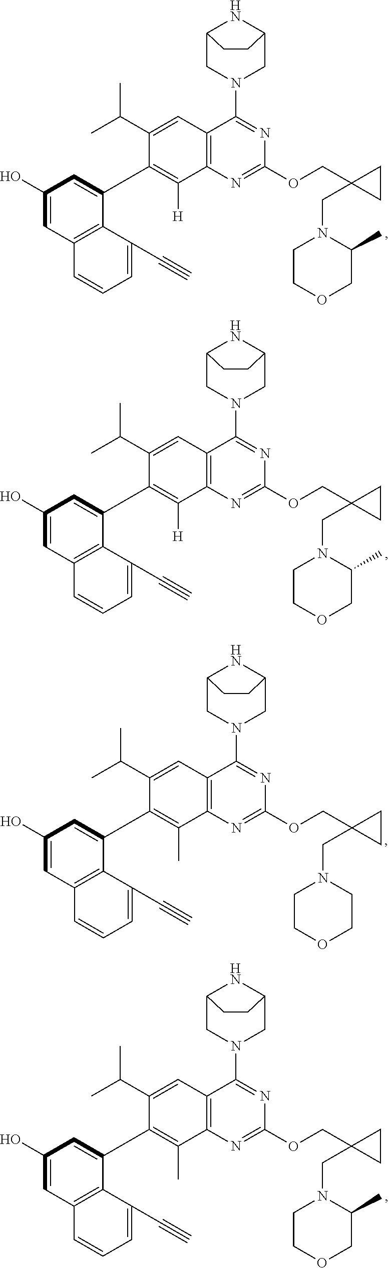

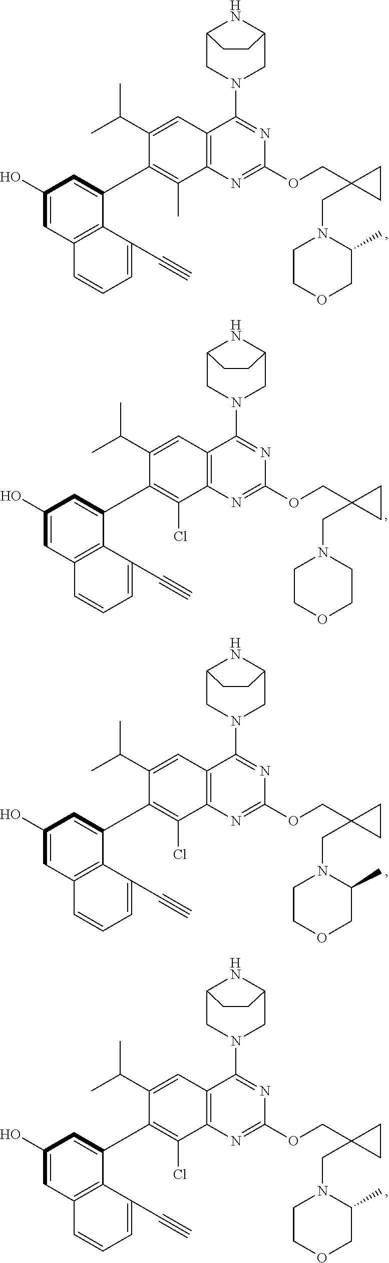

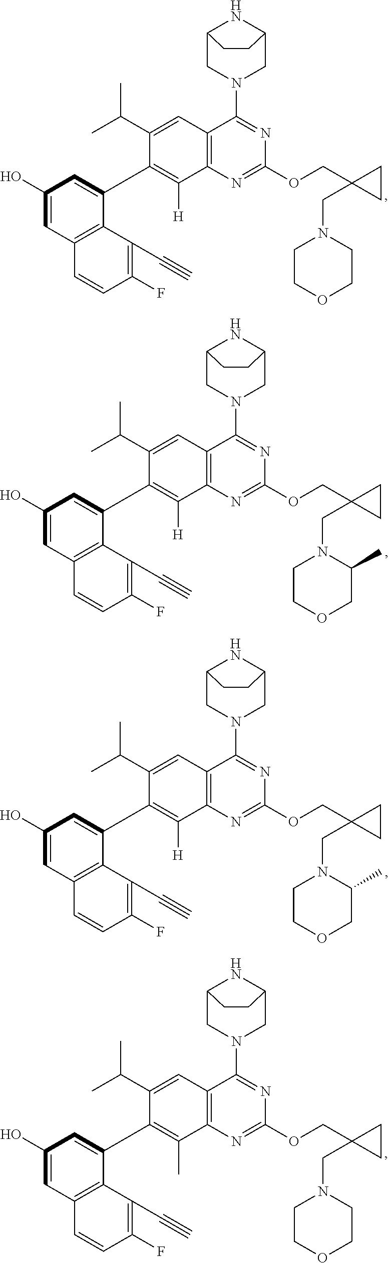

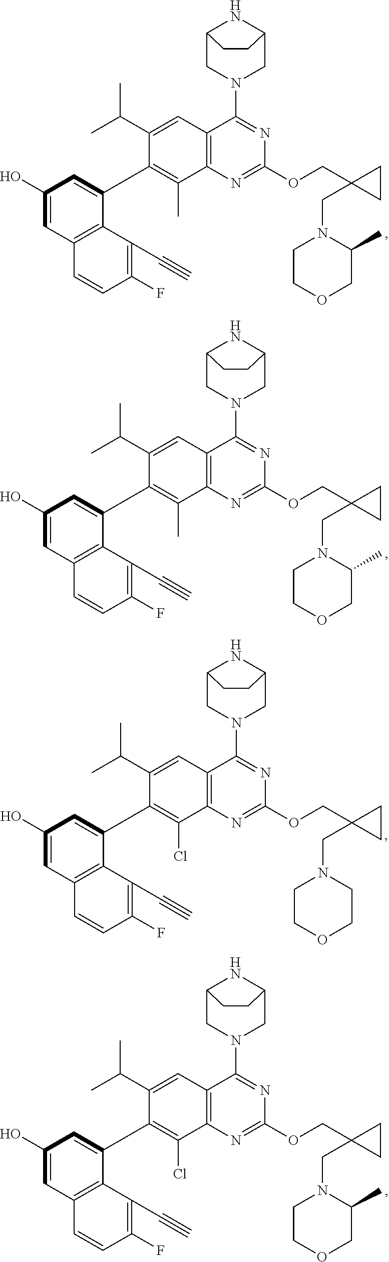

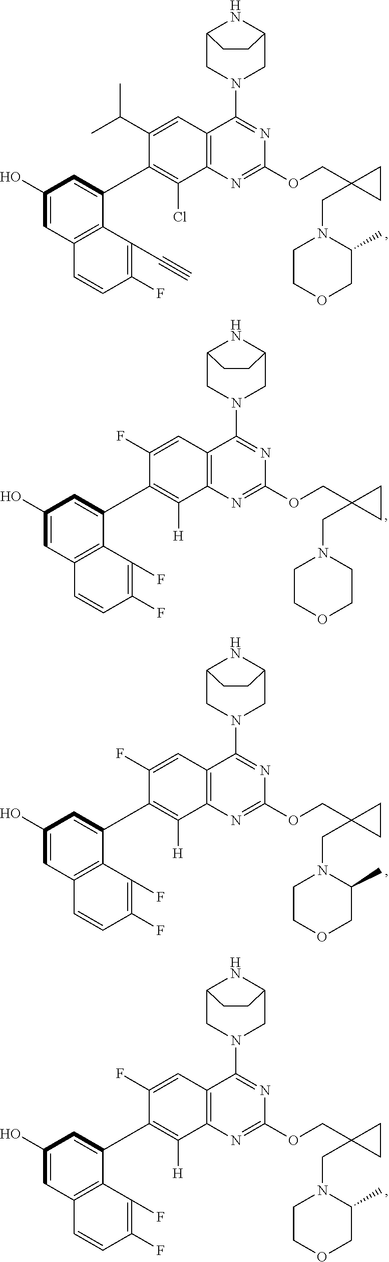

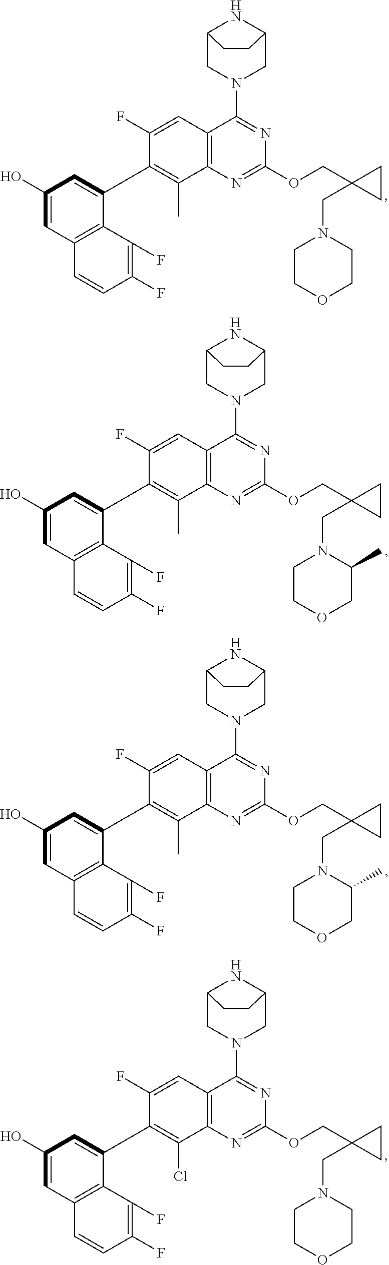

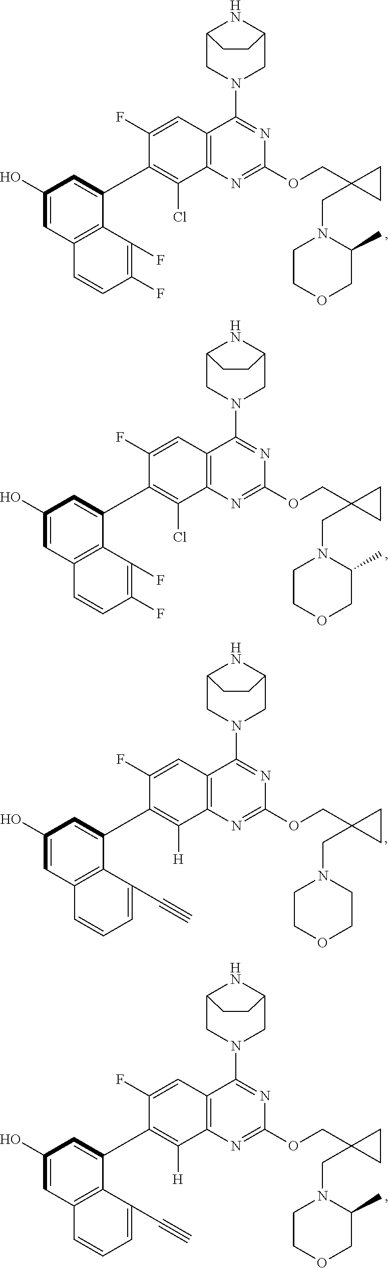

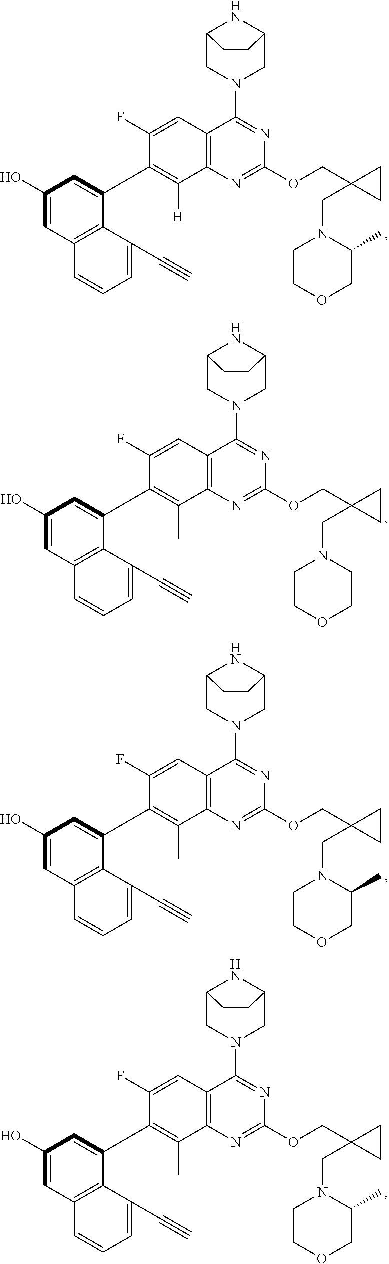

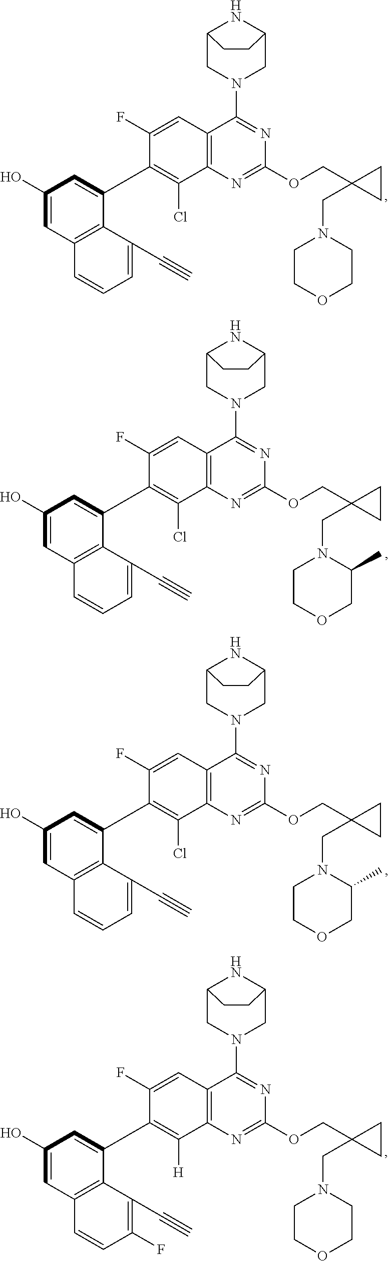

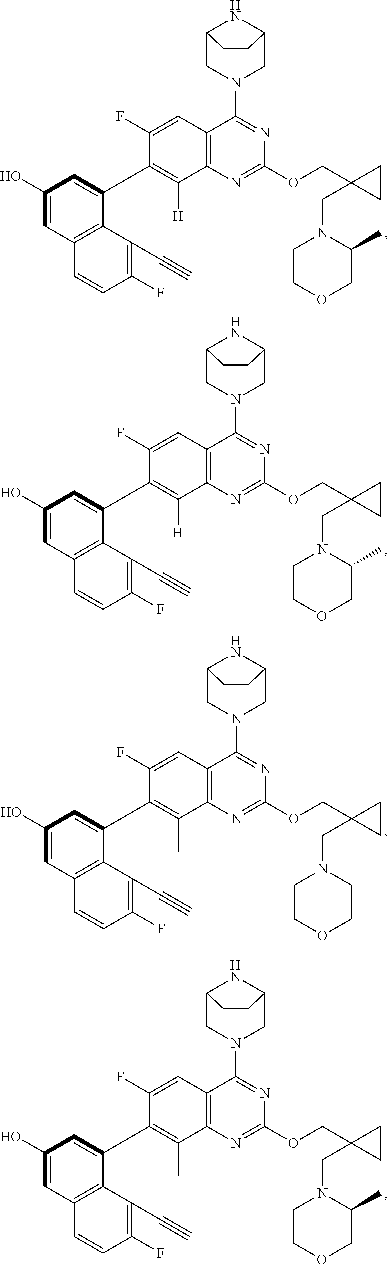

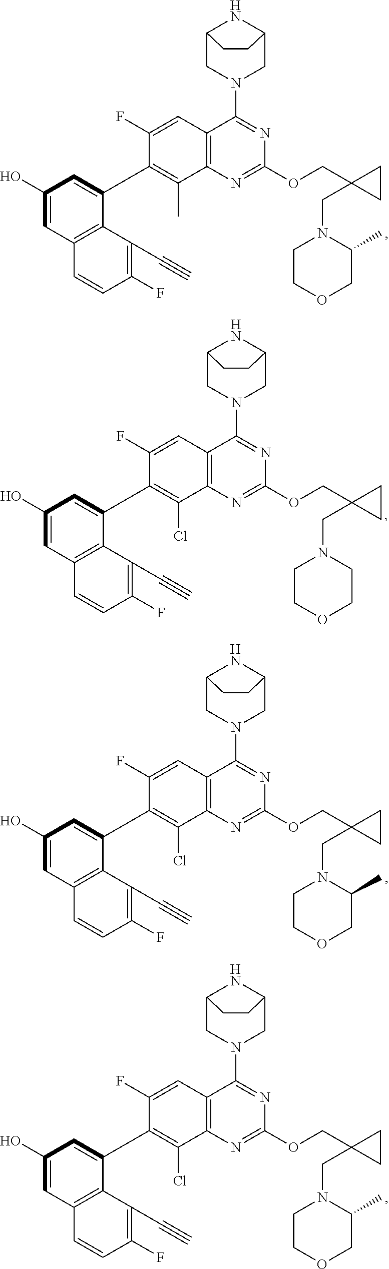

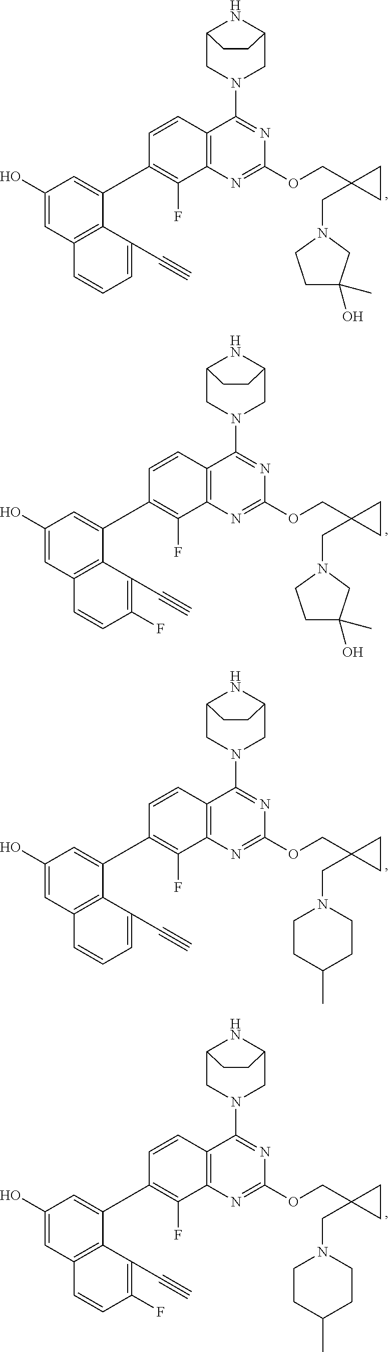 [SMILES: C#Cc1cccc2cc(O)cc(-c3ccc4c(N5CC6CCC(C5)N6)nc(OCC5(CN6CCC(C)(O)C6)CC5)nc4c3F)c12 ; C#Cc1c(F)ccc2cc(O)cc(-c3ccc4c(N5CC6CCC(C5)N6)nc(OCC5(CN6CCC(C)(O)C6)CC5)nc4c3F)c12 ; C#Cc1cccc2cc(O)cc(-c3ccc4c(N5CC6CCC(C5)N6)nc(OCC5(CN6CCC(C)CC6)CC5)nc4c3F)c12 ; C#Cc1c(F)ccc2cc(O)cc(-c3ccc4c(N5CC6CCC(C5)N6)nc(OCC5(CN6CCC(C)CC6)CC5)nc4c3F)c12]

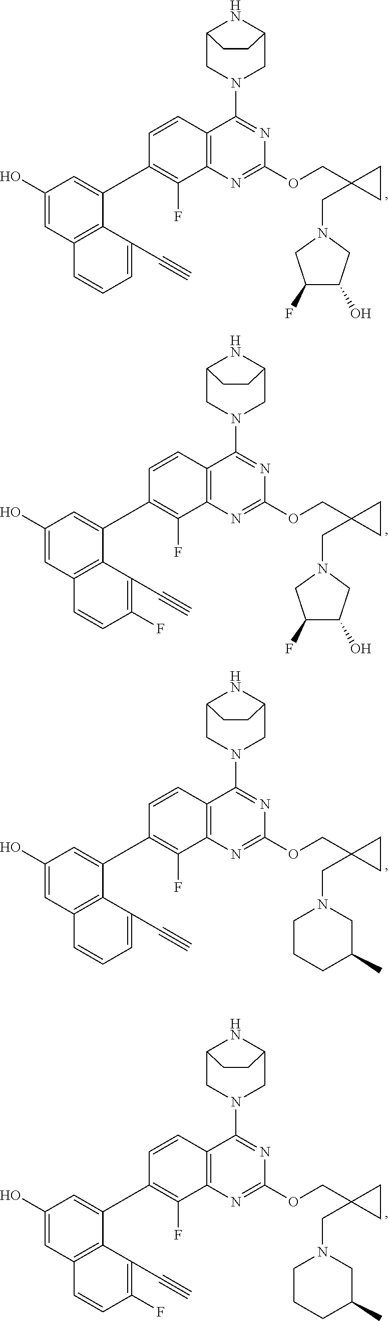 [SMILES: C#Cc1cccc2cc(O)cc(-c3ccc4c(N5CC6CCC(C5)N6)nc(OCC5(CN6C[C@H](O)[C@@H](F)C6)CC5)nc4c3F)c12 ; C#Cc1c(F)ccc2cc(O)cc(-c3ccc4c(N5CC6CCC(C5)N6)nc(OCC5(CN6C[C@H](O)[C@@H](F)C6)CC5)nc4c3F)c12 ; C#Cc1cccc2cc(O)cc(-c3ccc4c(N5CC6CCC(C5)N6)nc(OCC5(CN6CCC[C@H](C)C6)CC5)nc4c3F)c12 ; C#Cc1c(F)ccc2cc(O)cc(-c3ccc4c(N5CC6CCC(C5)N6)nc(OCC5(CN6CCC[C@H](C)C6)CC5)nc4c3F)c12]

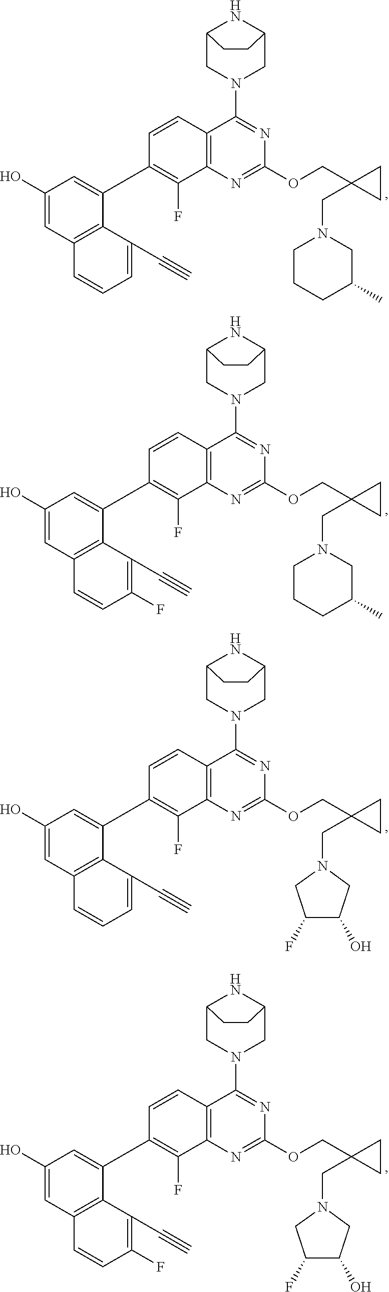 [SMILES: C#Cc1cccc2cc(O)cc(-c3ccc4c(N5CC6CCC(C5)N6)nc(OCC5(CN6CCC[C@@H](C)C6)CC5)nc4c3F)c12 ; C#Cc1c(F)ccc2cc(O)cc(-c3ccc4c(N5CC6CCC(C5)N6)nc(OCC5(CN6CCC[C@@H](C)C6)CC5)nc4c3F)c12 ; C#Cc1cccc2cc(O)cc(-c3ccc4c(N5CC6CCC(C5)N6)nc(OCC5(CN6C[C@@H](F)[C@@H](O)C6)CC5)nc4c3F)c12 ; C#Cc1c(F)ccc2cc(O)cc(-c3ccc4c(N5CC6CCC(C5)N6)nc(OCC5(CN6C[C@@H](F)[C@@H](O)C6)CC5)nc4c3F)c12]

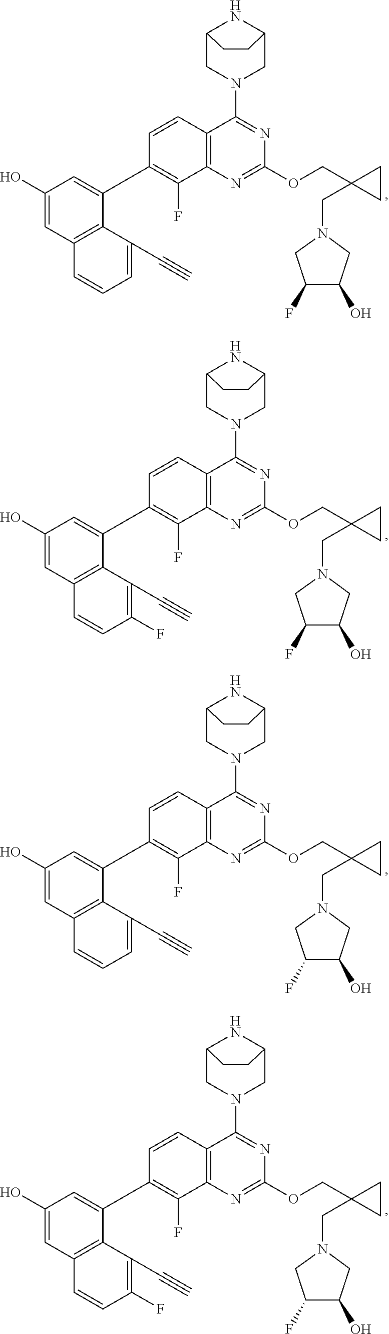 [SMILES: C#Cc1cccc2cc(O)cc(-c3ccc4c(N5CC6CCC(C5)N6)nc(OCC5(CN6C[C@@H](O)[C@@H](F)C6)CC5)nc4c3F)c12 ; C#Cc1c(F)ccc2cc(O)cc(-c3ccc4c(N5CC6CCC(C5)N6)nc(OCC5(CN6C[C@@H](O)[C@@H](F)C6)CC5)nc4c3F)c12 ; C#Cc1cccc2cc(O)cc(-c3ccc4c(N5CC6CCC(C5)N6)nc(OCC5(CN6C[C@@H](O)[C@H](F)C6)CC5)nc4c3F)c12 ; C#Cc1c(F)ccc2cc(O)cc(-c3ccc4c(N5CC6CCC(C5)N6)nc(OCC5(CN6C[C@@H](O)[C@H](F)C6)CC5)nc4c3F)c12]

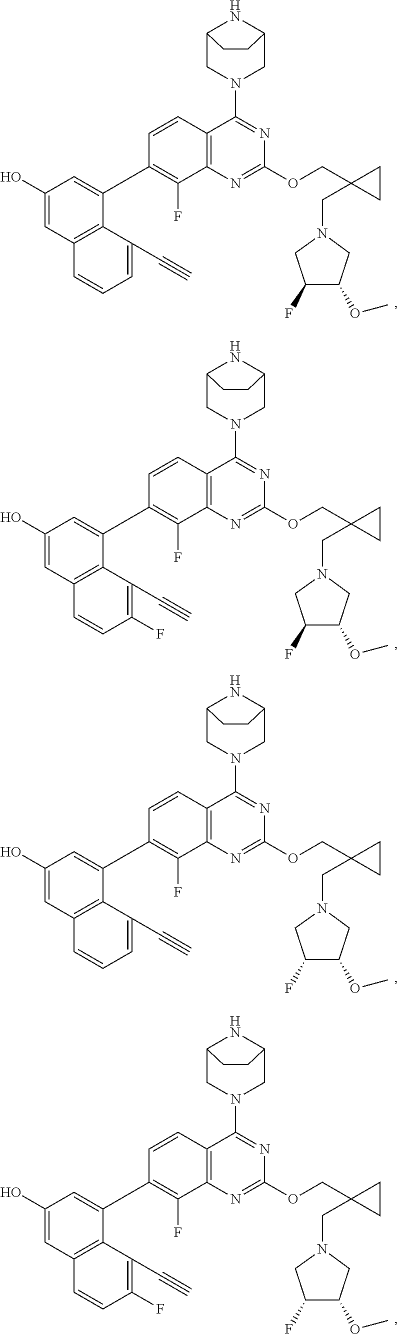 [SMILES: C#Cc1cccc2cc(O)cc(-c3ccc4c(N5CC6CCC(C5)N6)nc(OCC5(CN6C[C@H](OC)[C@@H](F)C6)CC5)nc4c3F)c12 ; C#Cc1c(F)ccc2cc(O)cc(-c3ccc4c(N5CC6CCC(C5)N6)nc(OCC5(CN6C[C@H](OC)[C@@H](F)C6)CC5)nc4c3F)c12 ; C#Cc1cccc2cc(O)cc(-c3ccc4c(N5CC6CCC(C5)N6)nc(OCC5(CN6C[C@@H](F)[C@@H](OC)C6)CC5)nc4c3F)c12 ; C#Cc1c(F)ccc2cc(O)cc(-c3ccc4c(N5CC6CCC(C5)N6)nc(OCC5(CN6C[C@@H](F)[C@@H](OC)C6)CC5)nc4c3F)c12]

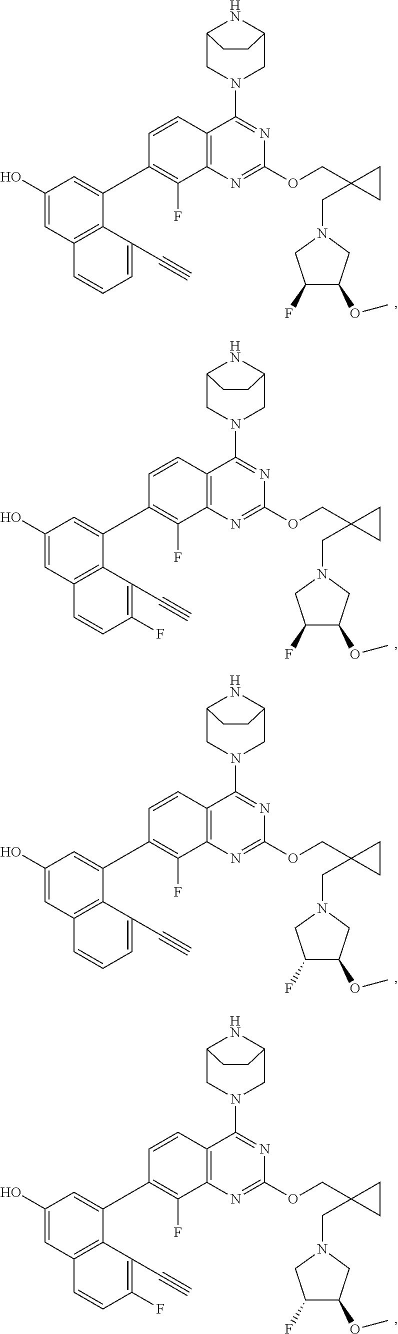 [SMILES: C#Cc1cccc2cc(O)cc(-c3ccc4c(N5CC6CCC(C5)N6)nc(OCC5(CN6C[C@H](F)[C@H](OC)C6)CC5)nc4c3F)c12 ; C#Cc1c(F)ccc2cc(O)cc(-c3ccc4c(N5CC6CCC(C5)N6)nc(OCC5(CN6C[C@H](F)[C@H](OC)C6)CC5)nc4c3F)c12 ; C#Cc1cccc2cc(O)cc(-c3ccc4c(N5CC6CCC(C5)N6)nc(OCC5(CN6C[C@@H](F)[C@H](OC)C6)CC5)nc4c3F)c12 ; C#Cc1c(F)ccc2cc(O)cc(-c3ccc4c(N5CC6CCC(C5)N6)nc(OCC5(CN6C[C@@H](F)[C@H](OC)C6)CC5)nc4c3F)c12]

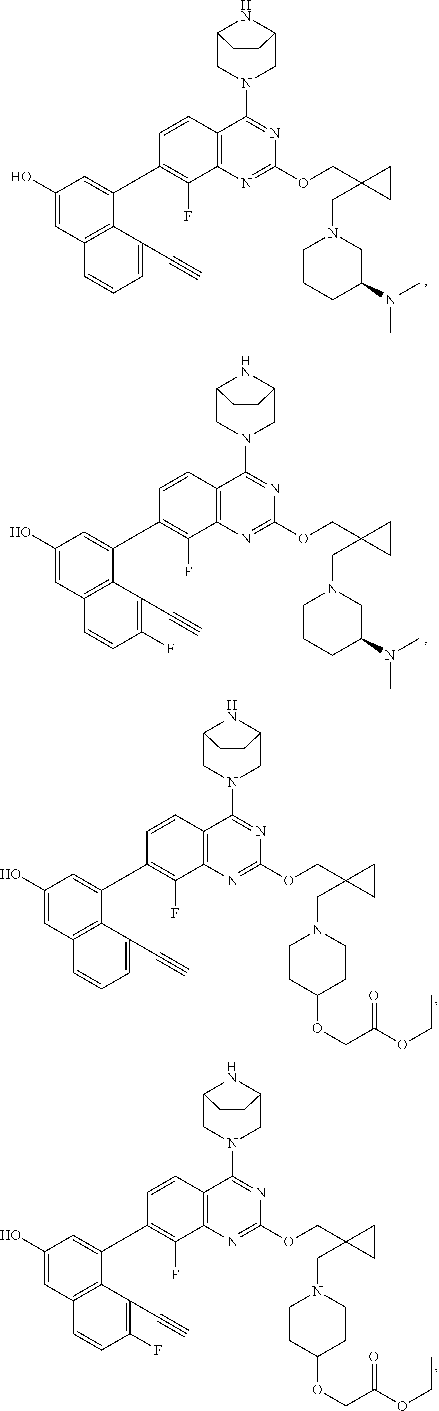 [SMILES: C#Cc1cccc2cc(O)cc(-c3ccc4c(N5CC6CCC(C5)N6)nc(OCC5(CN6CCC[C@H](N(C)C)C6)CC5)nc4c3F)c12 ; C#Cc1c(F)ccc2cc(O)cc(-c3ccc4c(N5CC6CCC(C5)N6)nc(OCC5(CN6CCC[C@H](N(C)C)C6)CC5)nc4c3F)c12 ; C#Cc1cccc2cc(O)cc(-c3ccc4c(N5CC6CCC(C5)N6)nc(OCC5(CN6CCC(OCC(=O)OCC)CC6)CC5)nc4c3F)c12 ; C#Cc1c(F)ccc2cc(O)cc(-c3ccc4c(N5CC6CCC(C5)N6)nc(OCC5(CN6CCC(OCC(=O)OCC)CC6)CC5)nc4c3F)c12]

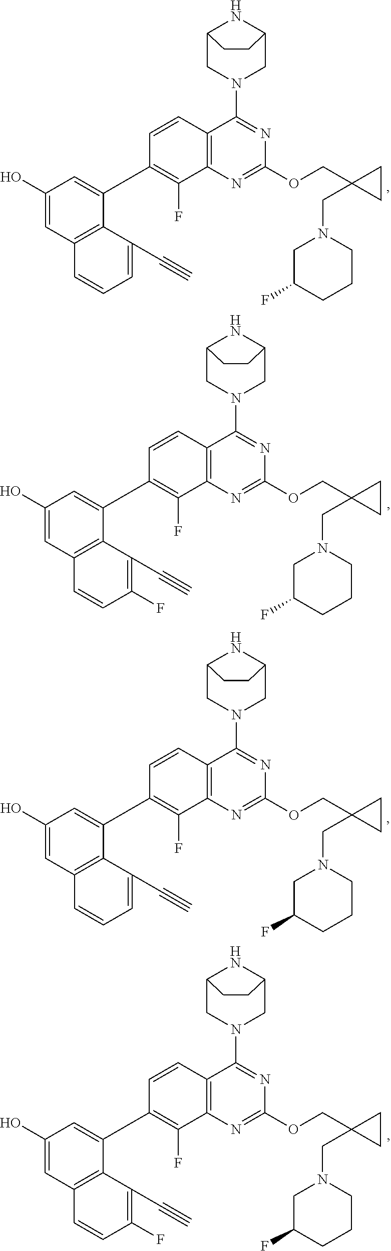

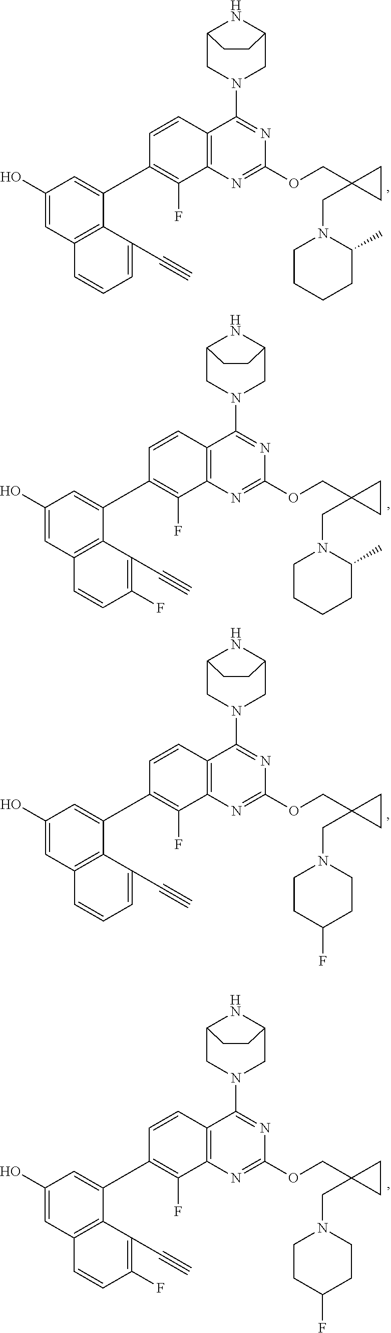 [SMILES: C#Cc1cccc2cc(O)cc(-c3ccc4c(N5CC6CCC(C5)N6)nc(OCC5(CN6CCCC[C@H]6C)CC5)nc4c3F)c12 ; C#Cc1c(F)ccc2cc(O)cc(-c3ccc4c(N5CC6CCC(C5)N6)nc(OCC5(CN6CCCC[C@H]6C)CC5)nc4c3F)c12 ; C#Cc1cccc2cc(O)cc(-c3ccc4c(N5CC6CCC(C5)N6)nc(OCC5(CN6CCC(F)CC6)CC5)nc4c3F)c12 ; C#Cc1c(F)ccc2cc(O)cc(-c3ccc4c(N5CC6CCC(C5)N6)nc(OCC5(CN6CCC(F)CC6)CC5)nc4c3F)c12]

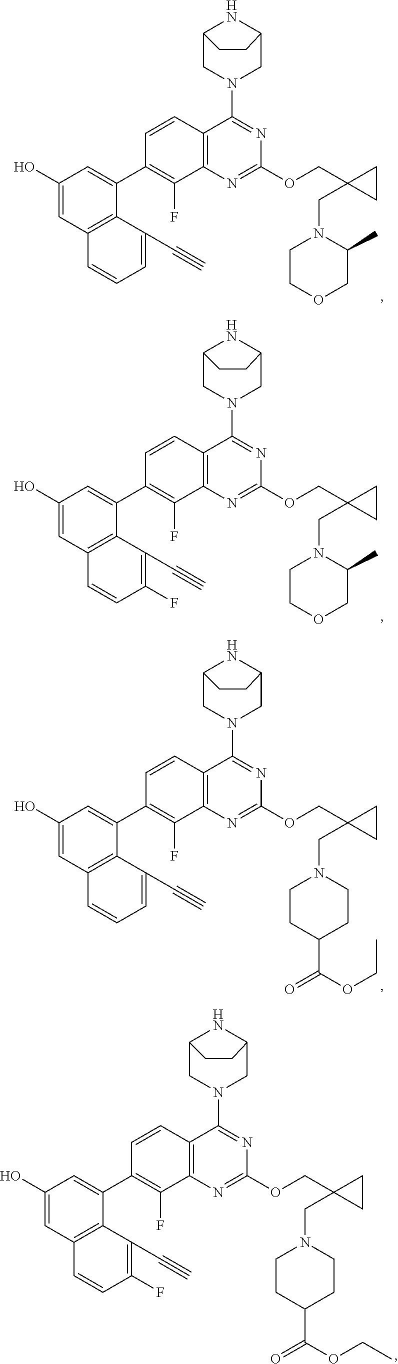 [SMILES: C#Cc1cccc2cc(O)cc(-c3ccc4c(N5CC6CCC(C5)N6)nc(OCC5(CN6CCOC[C@@H]6C)CC5)nc4c3F)c12 ; C#Cc1c(F)ccc2cc(O)cc(-c3ccc4c(N5CC6CCC(C5)N6)nc(OCC5(CN6CCOC[C@@H]6C)CC5)nc4c3F)c12 ; C#Cc1cccc2cc(O)cc(-c3ccc4c(N5CC6CCC(C5)N6)nc(OCC5(CN6CCC(C(=O)OCC)CC6)CC5)nc4c3F)c12 ; C#Cc1c(F)ccc2cc(O)cc(-c3ccc4c(N5CC6CCC(C5)N6)nc(OCC5(CN6CCC(C(=O)OCC)CC6)CC5)nc4c3F)c12]

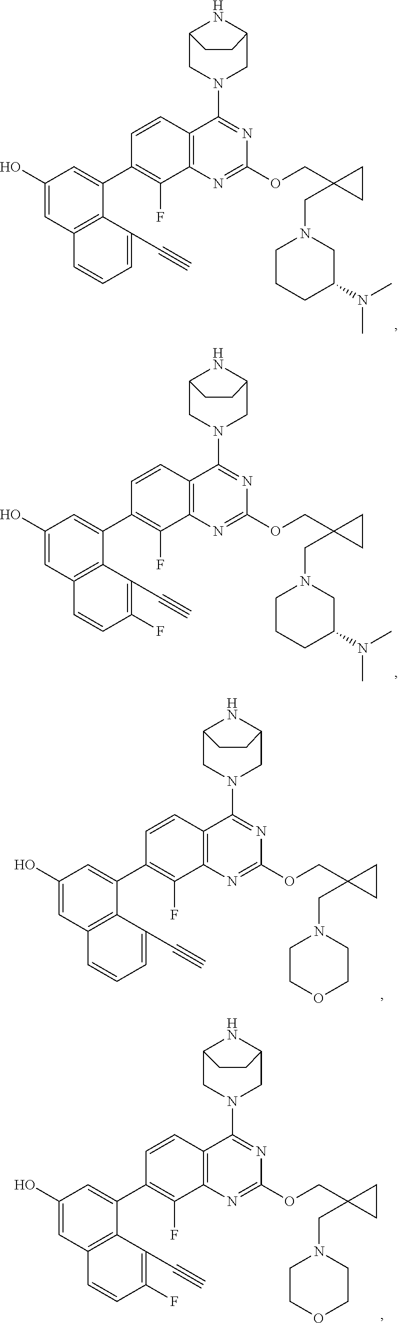 [SMILES: C#Cc1cccc2cc(O)cc(-c3ccc4c(N5CC6CCC(C5)N6)nc(OCC5(CN6CCC[C@@H](N(C)C)C6)CC5)nc4c3F)c12 ; C#Cc1c(F)ccc2cc(O)cc(-c3ccc4c(N5CC6CCC(C5)N6)nc(OCC5(CN6CCC[C@@H](N(C)C)C6)CC5)nc4c3F)c12 ; C#Cc1cccc2cc(O)cc(-c3ccc4c(N5CC6CCC(C5)N6)nc(OCC5(CN6CCOCC6)CC5)nc4c3F)c12 ; C#Cc1c(F)ccc2cc(O)cc(-c3ccc4c(N5CC6CCC(C5)N6)nc(OCC5(CN6CCOCC6)CC5)nc4c3F)c12]

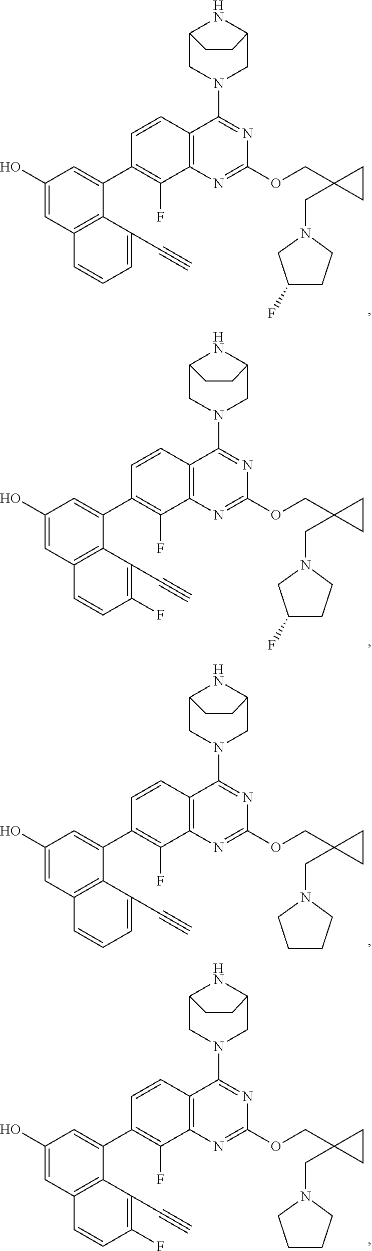 [SMILES: C#Cc1cccc2cc(O)cc(-c3ccc4c(N5CC6CCC(C5)N6)nc(OCC5(CN6CC[C@H](F)C6)CC5)nc4c3F)c12 ; C#Cc1c(F)ccc2cc(O)cc(-c3ccc4c(N5CC6CCC(C5)N6)nc(OCC5(CN6CC[C@H](F)C6)CC5)nc4c3F)c12 ; C#Cc1cccc2cc(O)cc(-c3ccc4c(N5CC6CCC(C5)N6)nc(OCC5(CN6CCCC6)CC5)nc4c3F)c12 ; C#Cc1c(F)ccc2cc(O)cc(-c3ccc4c(N5CC6CCC(C5)N6)nc(OCC5(CN6CCCC6)CC5)nc4c3F)c12]

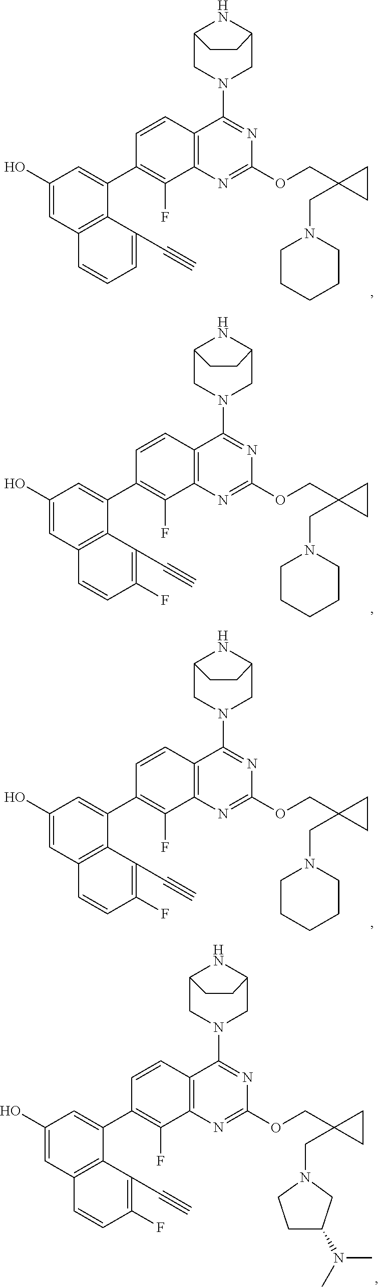 [SMILES: C#Cc1cccc2cc(O)cc(-c3ccc4c(N5CC6CCC(C5)N6)nc(OCC5(CN6CCCCC6)CC5)nc4c3F)c12 ; C#Cc1c(F)ccc2cc(O)cc(-c3ccc4c(N5CC6CCC(C5)N6)nc(OCC5(CN6CCCCC6)CC5)nc4c3F)c12 ; C#Cc1c(F)ccc2cc(O)cc(-c3ccc4c(N5CC6CCC(C5)N6)nc(OCC5(CN6CCCCC6)CC5)nc4c3F)c12 ; C#Cc1c(F)ccc2cc(O)cc(-c3ccc4c(N5CC6CCC(C5)N6)nc(OCC5(CN6CC[C@@H](N(C)C)C6)CC5)nc4c3F)c12]

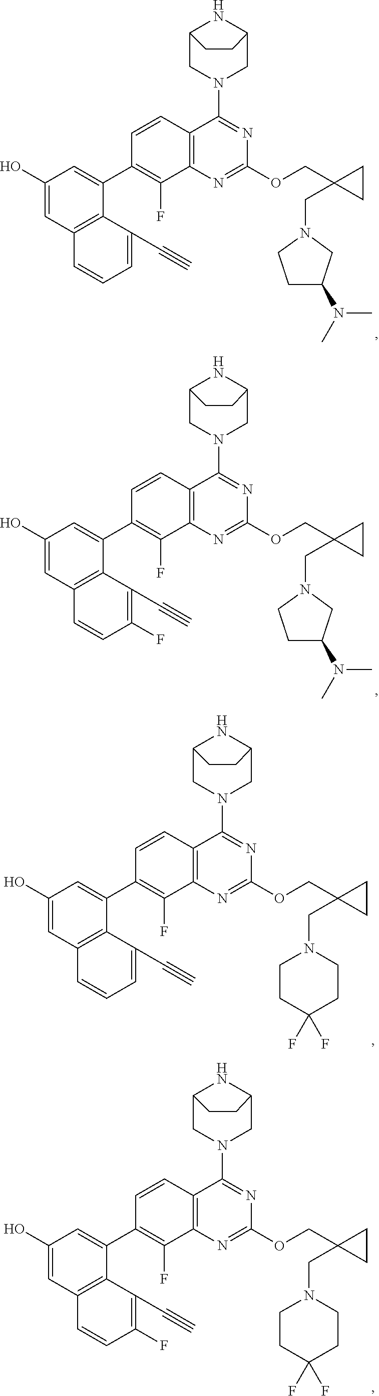 [SMILES: C#Cc1cccc2cc(O)cc(-c3ccc4c(N5CC6CCC(C5)N6)nc(OCC5(CN6CC[C@H](N(C)C)C6)CC5)nc4c3F)c12 ; C#Cc1c(F)ccc2cc(O)cc(-c3ccc4c(N5CC6CCC(C5)N6)nc(OCC5(CN6CC[C@H](N(C)C)C6)CC5)nc4c3F)c12 ; C#Cc1cccc2cc(O)cc(-c3ccc4c(N5CC6CCC(C5)N6)nc(OCC5(CN6CCC(F)(F)CC6)CC5)nc4c3F)c12 ; C#Cc1c(F)ccc2cc(O)cc(-c3ccc4c(N5CC6CCC(C5)N6)nc(OCC5(CN6CCC(F)(F)CC6)CC5)nc4c3F)c12]

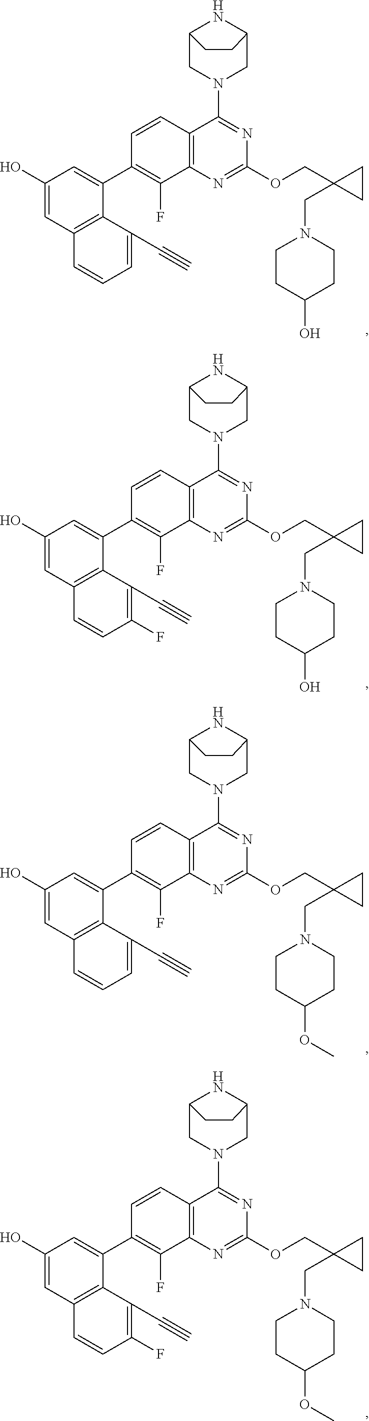 [SMILES: C#Cc1cccc2cc(O)cc(-c3ccc4c(N5CC6CCC(C5)N6)nc(OCC5(CN6CCC(O)CC6)CC5)nc4c3F)c12 ; C#Cc1c(F)ccc2cc(O)cc(-c3ccc4c(N5CC6CCC(C5)N6)nc(OCC5(CN6CCC(O)CC6)CC5)nc4c3F)c12 ; C#Cc1cccc2cc(O)cc(-c3ccc4c(N5CC6CCC(C5)N6)nc(OCC5(CN6CCC(OC)CC6)CC5)nc4c3F)c12 ; C#Cc1c(F)ccc2cc(O)cc(-c3ccc4c(N5CC6CCC(C5)N6)nc(OCC5(CN6CCC(OC)CC6)CC5)nc4c3F)c12]

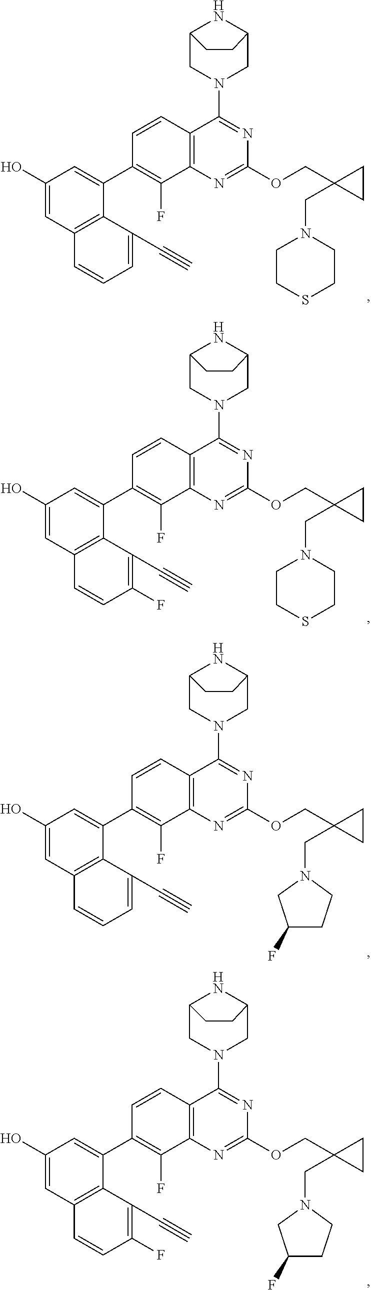 [SMILES: C#Cc1cccc2cc(O)cc(-c3ccc4c(N5CC6CCC(C5)N6)nc(OCC5(CN6CCSCC6)CC5)nc4c3F)c12 ; C#Cc1c(F)ccc2cc(O)cc(-c3ccc4c(N5CC6CCC(C5)N6)nc(OCC5(CN6CCSCC6)CC5)nc4c3F)c12 ; C#Cc1cccc2cc(O)cc(-c3ccc4c(N5CC6CCC(C5)N6)nc(OCC5(CN6CC[C@@H](F)C6)CC5)nc4c3F)c12 ; C#Cc1c(F)ccc2cc(O)cc(-c3ccc4c(N5CC6CCC(C5)N6)nc(OCC5(CN6CC[C@@H](F)C6)CC5)nc4c3F)c12]

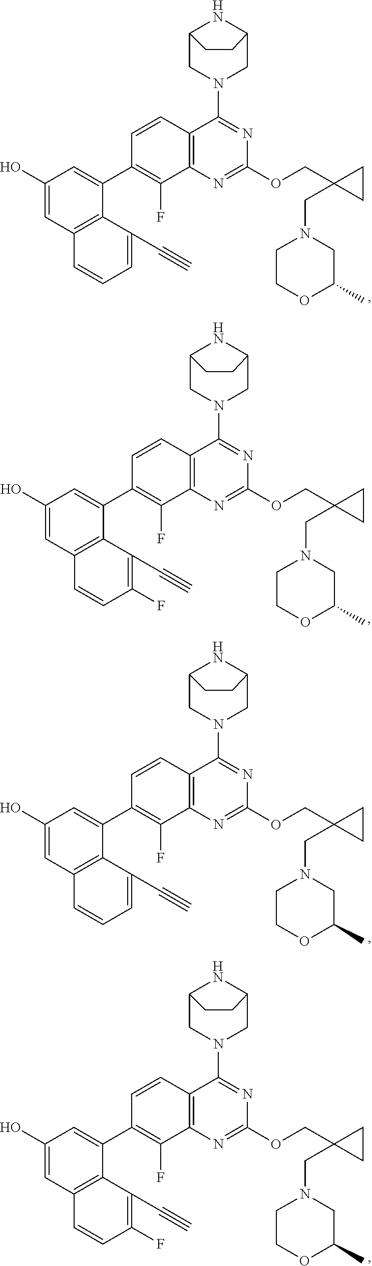 [SMILES: C#Cc1cccc2cc(O)cc(-c3ccc4c(N5CC6CCC(C5)N6)nc(OCC5(CN6CCO[C@@H](C)C6)CC5)nc4c3F)c12 ; C#Cc1c(F)ccc2cc(O)cc(-c3ccc4c(N5CC6CCC(C5)N6)nc(OCC5(CN6CCO[C@@H](C)C6)CC5)nc4c3F)c12 ; C#Cc1cccc2cc(O)cc(-c3ccc4c(N5CC6CCC(C5)N6)nc(OCC5(CN6CCO[C@H](C)C6)CC5)nc4c3F)c12 ; C#Cc1c(F)ccc2cc(O)cc(-c3ccc4c(N5CC6CCC(C5)N6)nc(OCC5(CN6CCO[C@H](C)C6)CC5)nc4c3F)c12]

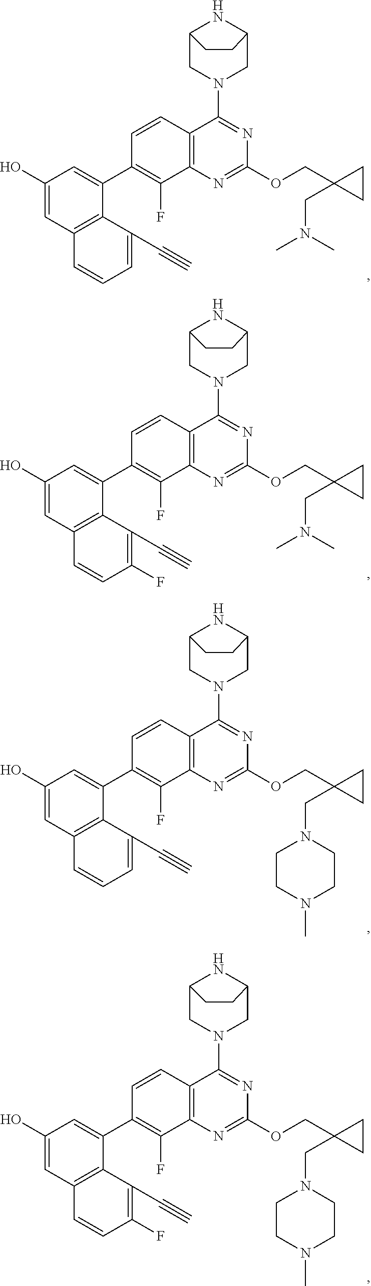 [SMILES: C#Cc1cccc2cc(O)cc(-c3ccc4c(N5CC6CCC(C5)N6)nc(OCC5(CN(C)C)CC5)nc4c3F)c12 ; C#Cc1c(F)ccc2cc(O)cc(-c3ccc4c(N5CC6CCC(C5)N6)nc(OCC5(CN(C)C)CC5)nc4c3F)c12 ; C#Cc1cccc2cc(O)cc(-c3ccc4c(N5CC6CCC(C5)N6)nc(OCC5(CN6CCN(C)CC6)CC5)nc4c3F)c12 ; C#Cc1c(F)ccc2cc(O)cc(-c3ccc4c(N5CC6CCC(C5)N6)nc(OCC5(CN6CCN(C)CC6)CC5)nc4c3F)c12]

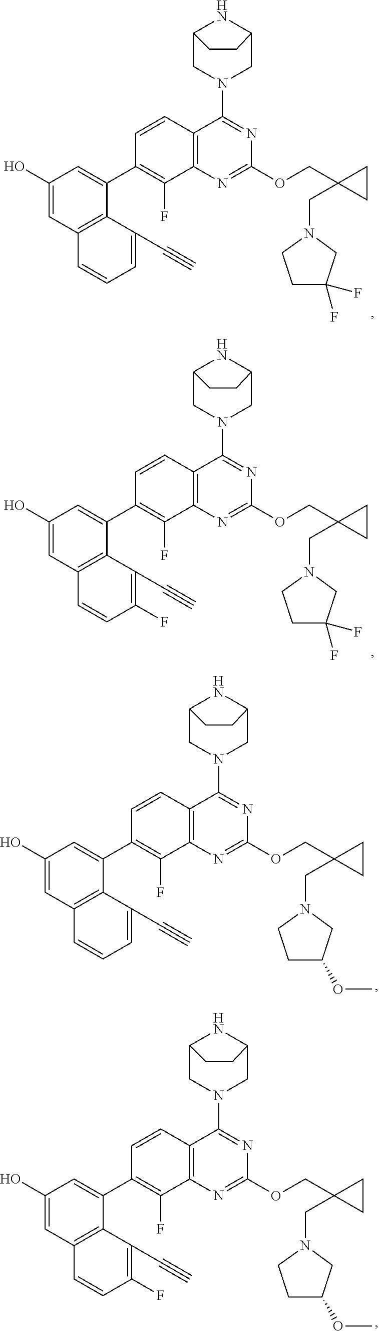 [SMILES: C#Cc1cccc2cc(O)cc(-c3ccc4c(N5CC6CCC(C5)N6)nc(OCC5(CN6CCC(F)(F)C6)CC5)nc4c3F)c12 ; C#Cc1c(F)ccc2cc(O)cc(-c3ccc4c(N5CC6CCC(C5)N6)nc(OCC5(CN6CCC(F)(F)C6)CC5)nc4c3F)c12 ; C#Cc1cccc2cc(O)cc(-c3ccc4c(N5CC6CCC(C5)N6)nc(OCC5(CN6CC[C@@H](OC)C6)CC5)nc4c3F)c12 ; C#Cc1c(F)ccc2cc(O)cc(-c3ccc4c(N5CC6CCC(C5)N6)nc(OCC5(CN6CC[C@@H](OC)C6)CC5)nc4c3F)c12]

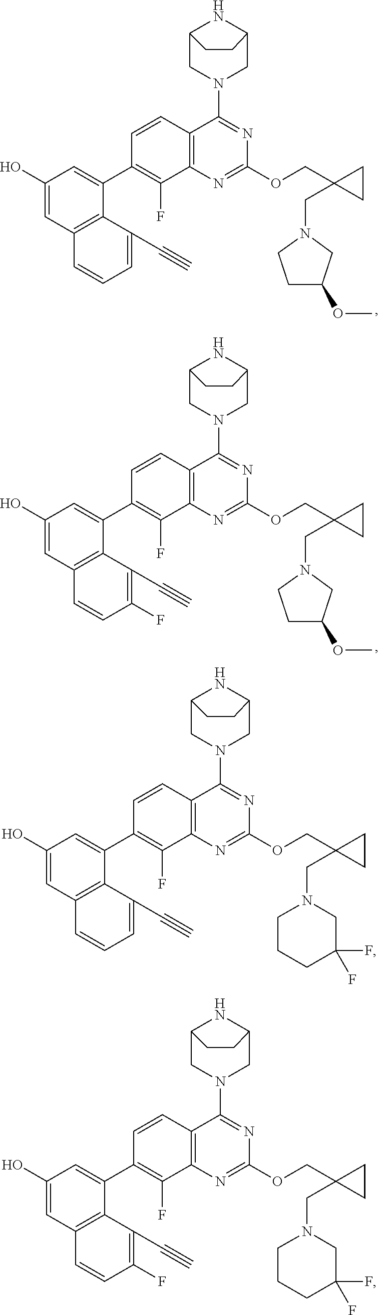 [SMILES: C#Cc1cccc2cc(O)cc(-c3ccc4c(N5CC6CCC(C5)N6)nc(OCC5(CN6CC[C@H](OC)C6)CC5)nc4c3F)c12 ; C#Cc1c(F)ccc2cc(O)cc(-c3ccc4c(N5CC6CCC(C5)N6)nc(OCC5(CN6CC[C@H](OC)C6)CC5)nc4c3F)c12 ; C#Cc1cccc2cc(O)cc(-c3ccc4c(N5CC6CCC(C5)N6)nc(OCC5(CN6CCCC(F)(F)C6)CC5)nc4c3F)c12 ; C#Cc1c(F)ccc2cc(O)cc(-c3ccc4c(N5CC6CCC(C5)N6)nc(OCC5(CN6CCCC(F)(F)C6)CC5)nc4c3F)c12]

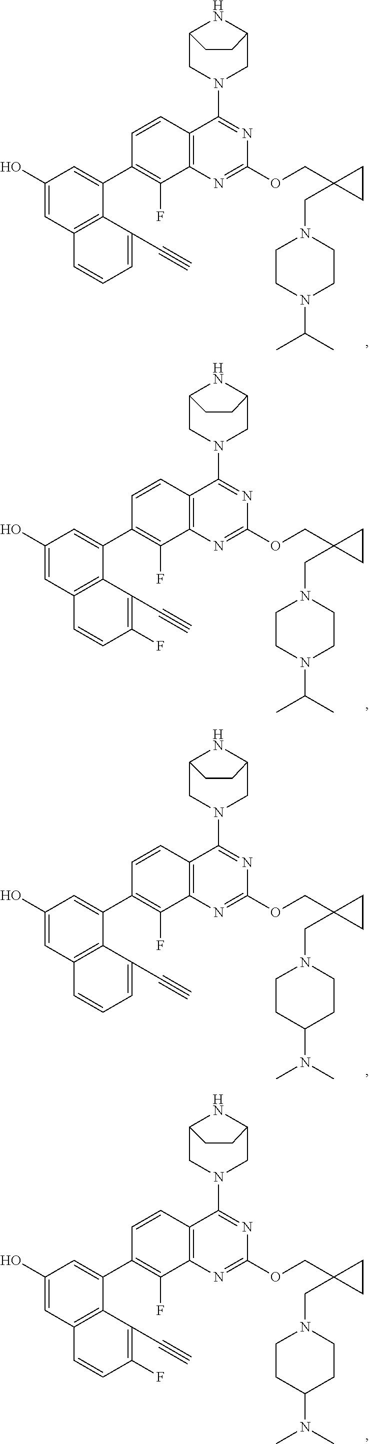 [SMILES: C#Cc1cccc2cc(O)cc(-c3ccc4c(N5CC6CCC(C5)N6)nc(OCC5(CN6CCN(C(C)C)CC6)CC5)nc4c3F)c12 ; C#Cc1c(F)ccc2cc(O)cc(-c3ccc4c(N5CC6CCC(C5)N6)nc(OCC5(CN6CCN(C(C)C)CC6)CC5)nc4c3F)c12 ; C#Cc1cccc2cc(O)cc(-c3ccc4c(N5CC6CCC(C5)N6)nc(OCC5(CN6CCC(N(C)C)CC6)CC5)nc4c3F)c12 ; C#Cc1c(F)ccc2cc(O)cc(-c3ccc4c(N5CC6CCC(C5)N6)nc(OCC5(CN6CCC(N(C)C)CC6)CC5)nc4c3F)c12]

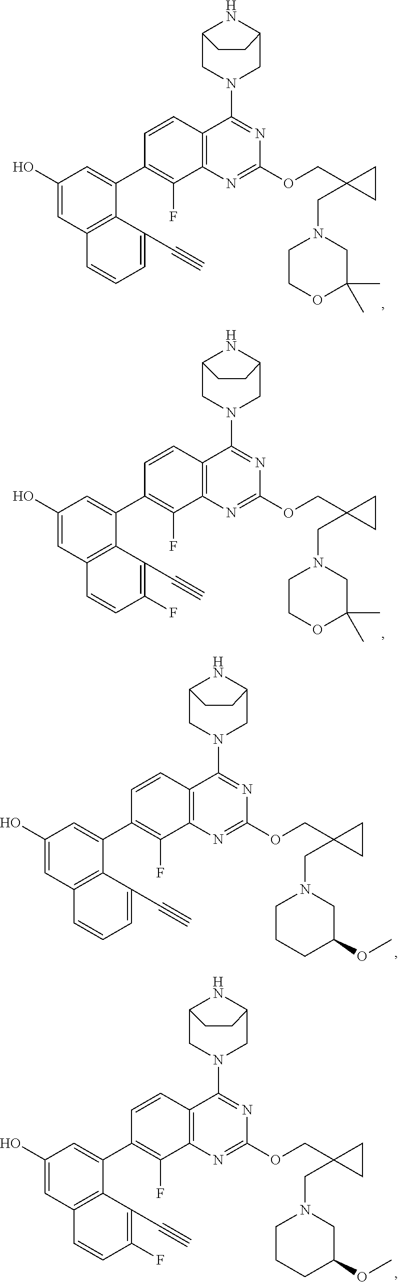 [SMILES: C#Cc1cccc2cc(O)cc(-c3ccc4c(N5CC6CCC(C5)N6)nc(OCC5(CN6CCOC(C)(C)C6)CC5)nc4c3F)c12 ; C#Cc1c(F)ccc2cc(O)cc(-c3ccc4c(N5CC6CCC(C5)N6)nc(OCC5(CN6CCOC(C)(C)C6)CC5)nc4c3F)c12 ; C#Cc1cccc2cc(O)cc(-c3ccc4c(N5CC6CCC(C5)N6)nc(OCC5(CN6CCC[C@H](OC)C6)CC5)nc4c3F)c12 ; C#Cc1c(F)ccc2cc(O)cc(-c3ccc4c(N5CC6CCC(C5)N6)nc(OCC5(CN6CCC[C@H](OC)C6)CC5)nc4c3F)c12]

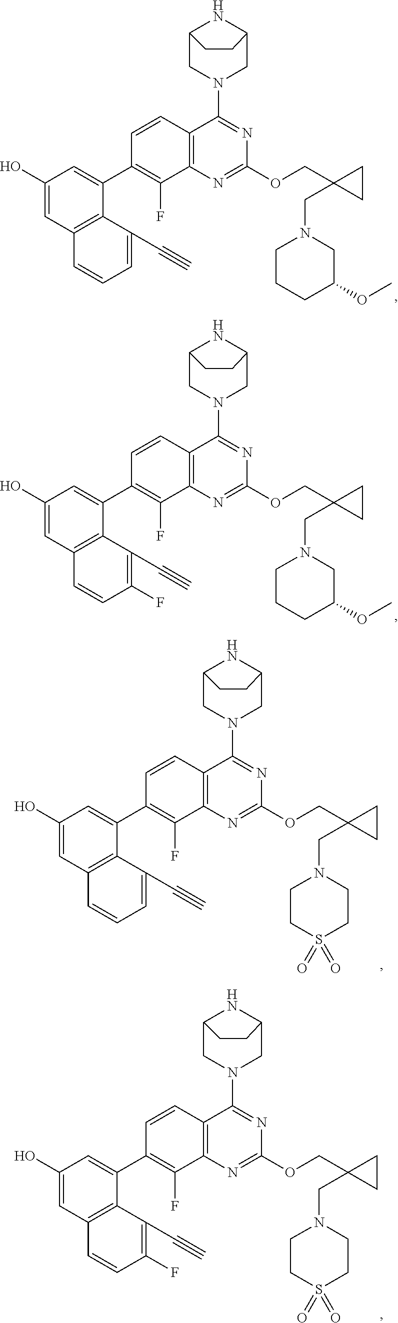 [SMILES: C#Cc1cccc2cc(O)cc(-c3ccc4c(N5CC6CCC(C5)N6)nc(OCC5(CN6CCC[C@@H](OC)C6)CC5)nc4c3F)c12 ; C#Cc1c(F)ccc2cc(O)cc(-c3ccc4c(N5CC6CCC(C5)N6)nc(OCC5(CN6CCC[C@@H](OC)C6)CC5)nc4c3F)c12 ; C#Cc1cccc2cc(O)cc(-c3ccc4c(N5CC6CCC(C5)N6)nc(OCC5(CN6CCS(=O)(=O)CC6)CC5)nc4c3F)c12 ; C#Cc1c(F)ccc2cc(O)cc(-c3ccc4c(N5CC6CCC(C5)N6)nc(OCC5(CN6CCS(=O)(=O)CC6)CC5)nc4c3F)c12]

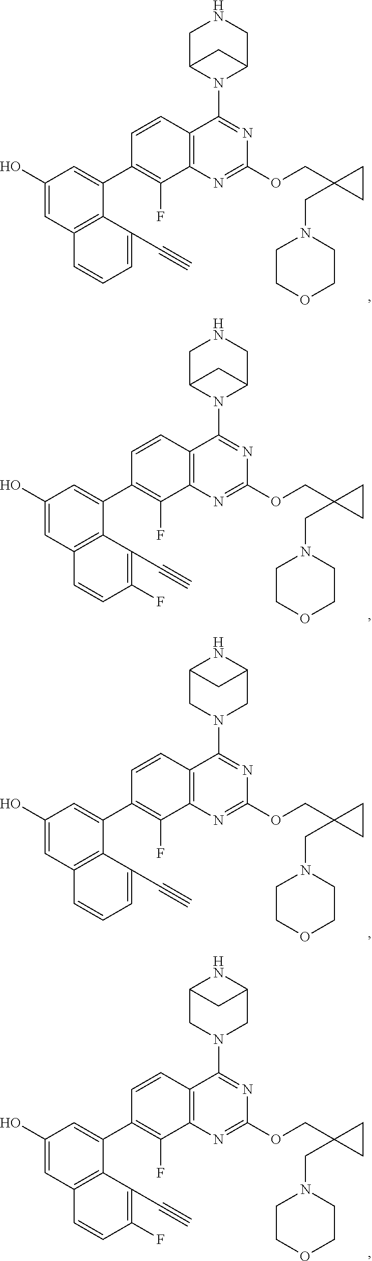 [SMILES: C#Cc1cccc2cc(O)cc(-c3ccc4c(N5C6CNCC5C6)nc(OCC5(CN6CCOCC6)CC5)nc4c3F)c12 ; C#Cc1c(F)ccc2cc(O)cc(-c3ccc4c(N5C6CNCC5C6)nc(OCC5(CN6CCOCC6)CC5)nc4c3F)c12 ; C#Cc1cccc2cc(O)cc(-c3ccc4c(N5CC6CC(C5)N6)nc(OCC5(CN6CCOCC6)CC5)nc4c3F)c12 ; C#Cc1c(F)ccc2cc(O)cc(-c3ccc4c(N5CC6CC(C5)N6)nc(OCC5(CN6CCOCC6)CC5)nc4c3F)c12]

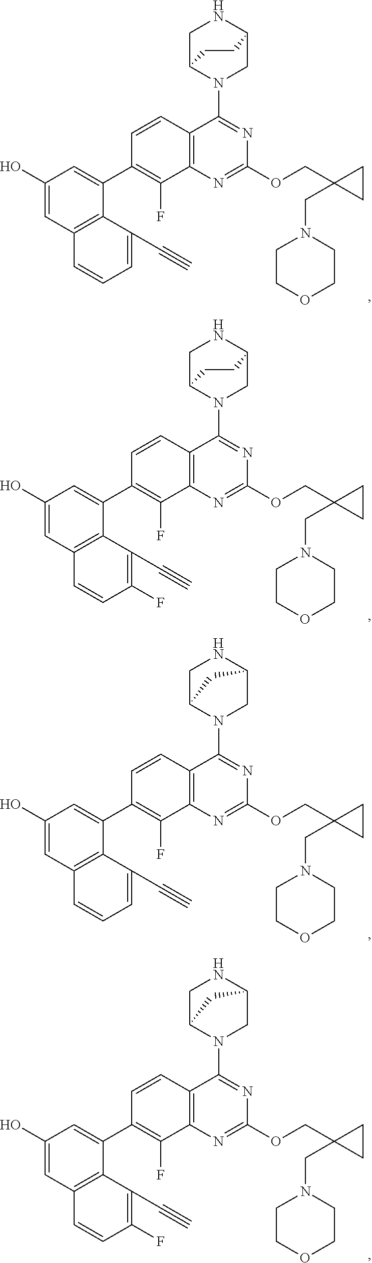 [SMILES: C#Cc1cccc2cc(O)cc(-c3ccc4c(N5C[C@H]6CC[C@@H]5CN6)nc(OCC5(CN6CCOCC6)CC5)nc4c3F)c12 ; C#Cc1c(F)ccc2cc(O)cc(-c3ccc4c(N5C[C@H]6CC[C@@H]5CN6)nc(OCC5(CN6CCOCC6)CC5)nc4c3F)c12 ; C#Cc1cccc2cc(O)cc(-c3ccc4c(N5C[C@H]6C[C@@H]5CN6)nc(OCC5(CN6CCOCC6)CC5)nc4c3F)c12 ; C#Cc1c(F)ccc2cc(O)cc(-c3ccc4c(N5C[C@H]6C[C@@H]5CN6)nc(OCC5(CN6CCOCC6)CC5)nc4c3F)c12]

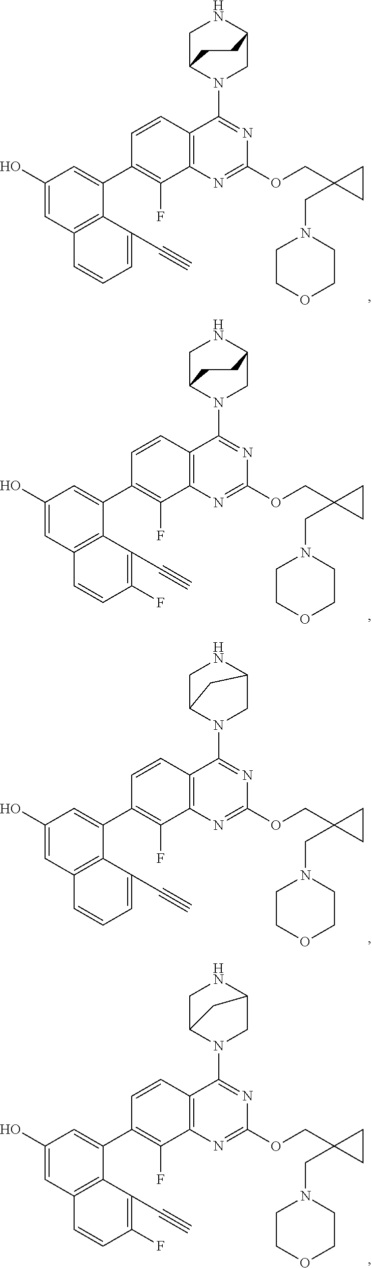 [SMILES: C#Cc1cccc2cc(O)cc(-c3ccc4c(N5C[C@@H]6CC[C@H]5CN6)nc(OCC5(CN6CCOCC6)CC5)nc4c3F)c12 ; C#Cc1c(F)ccc2cc(O)cc(-c3ccc4c(N5C[C@@H]6CC[C@H]5CN6)nc(OCC5(CN6CCOCC6)CC5)nc4c3F)c12 ; C#Cc1cccc2cc(O)cc(-c3ccc4c(N5CC6CC5CN6)nc(OCC5(CN6CCOCC6)CC5)nc4c3F)c12 ; C#Cc1c(F)ccc2cc(O)cc(-c3ccc4c(N5CC6CC5CN6)nc(OCC5(CN6CCOCC6)CC5)nc4c3F)c12]

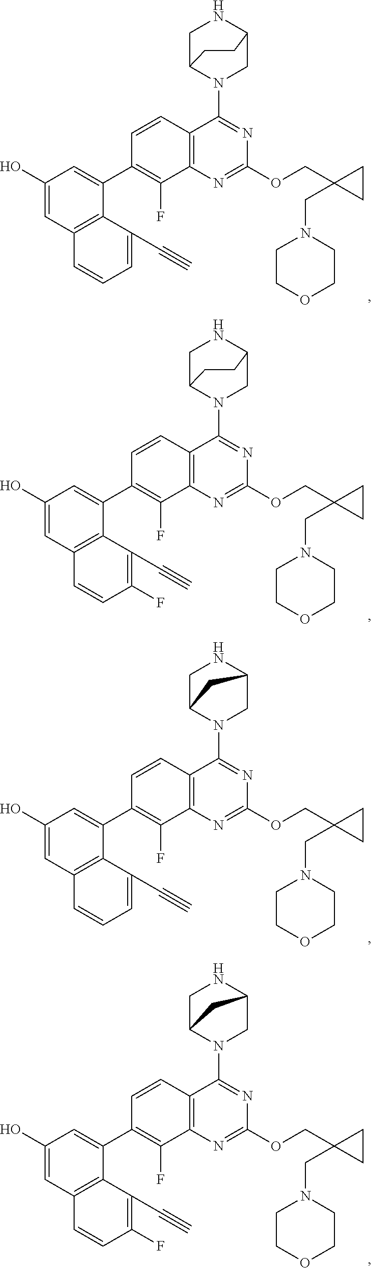 [SMILES: C#Cc1cccc2cc(O)cc(-c3ccc4c(N5CC6CCC5CN6)nc(OCC5(CN6CCOCC6)CC5)nc4c3F)c12 ; C#Cc1c(F)ccc2cc(O)cc(-c3ccc4c(N5CC6CCC5CN6)nc(OCC5(CN6CCOCC6)CC5)nc4c3F)c12 ; C#Cc1cccc2cc(O)cc(-c3ccc4c(N5C[C@@H]6C[C@H]5CN6)nc(OCC5(CN6CCOCC6)CC5)nc4c3F)c12 ; C#Cc1c(F)ccc2cc(O)cc(-c3ccc4c(N5C[C@@H]6C[C@H]5CN6)nc(OCC5(CN6CCOCC6)CC5)nc4c3F)c12]

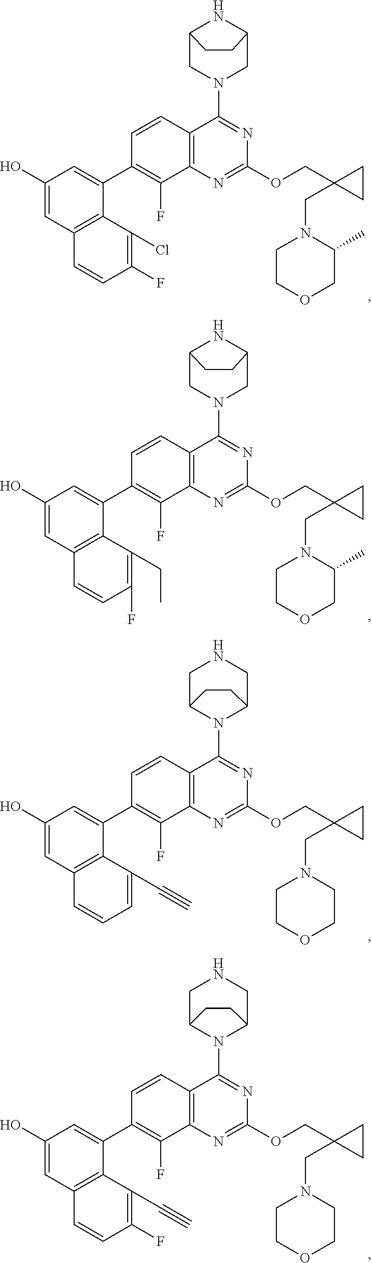 [SMILES: C[C@@H]1COCCN1CC1(COc2nc(N3CC4CCC(C3)N4)c3ccc(-c4cc(O)cc5ccc(F)c(Cl)c45)c(F)c3n2)CC1 ; CCc1c(F)ccc2cc(O)cc(-c3ccc4c(N5CC6CCC(C5)N6)nc(OCC5(CN6CCOC[C@H]6C)CC5)nc4c3F)c12 ; C#Cc1cccc2cc(O)cc(-c3ccc4c(N5C6CCC5CNC6)nc(OCC5(CN6CCOCC6)CC5)nc4c3F)c12 ; C#Cc1c(F)ccc2cc(O)cc(-c3ccc4c(N5C6CCC5CNC6)nc(OCC5(CN6CCOCC6)CC5)nc4c3F)c12]

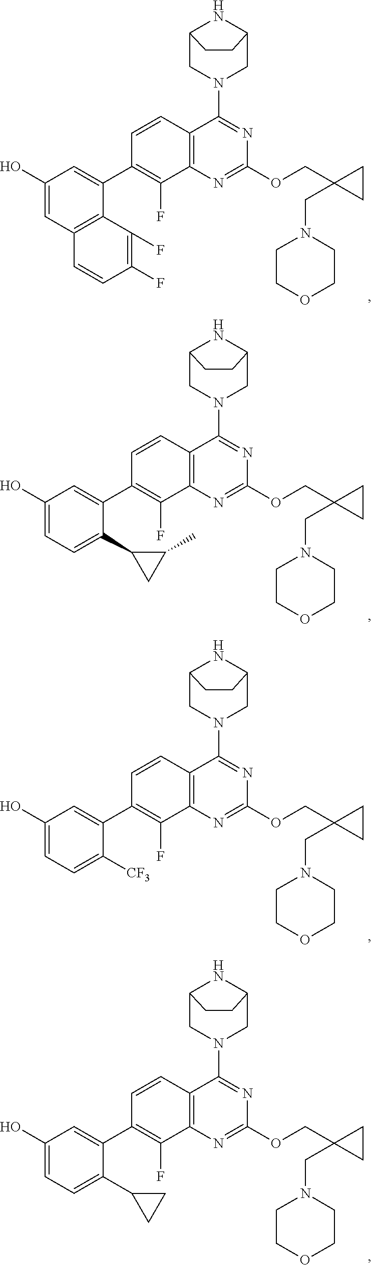 [SMILES: Oc1cc(-c2ccc3c(N4CC5CCC(C4)N5)nc(OCC4(CN5CCOCC5)CC4)nc3c2F)c2c(F)c(F)ccc2c1 ; C[C@@H]1C[C@H]1c1ccc(O)cc1-c1ccc2c(N3CC4CCC(C3)N4)nc(OCC3(CN4CCOCC4)CC3)nc2c1F ; Oc1ccc(C(F)(F)F)c(-c2ccc3c(N4CC5CCC(C4)N5)nc(OCC4(CN5CCOCC5)CC4)nc3c2F)c1 ; Oc1ccc(C2CC2)c(-c2ccc3c(N4CC5CCC(C4)N5)nc(OCC4(CN5CCOCC5)CC4)nc3c2F)c1]

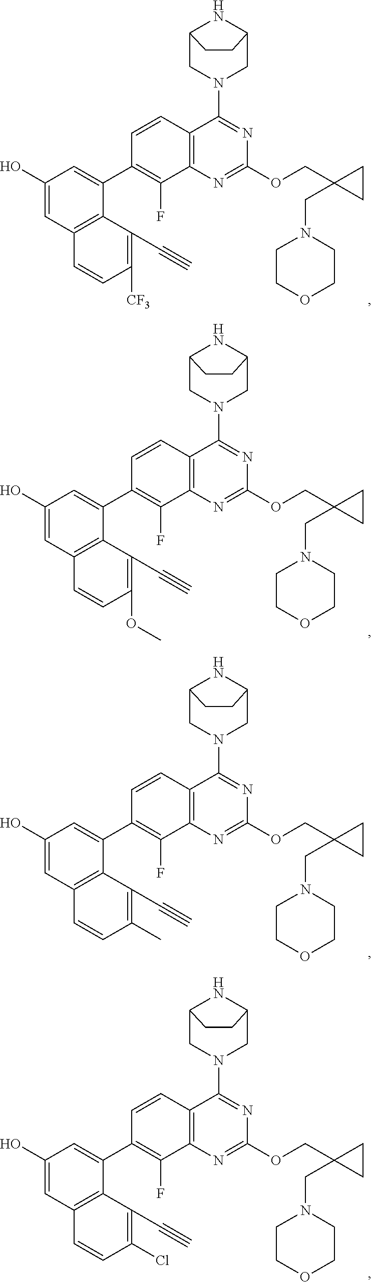 [SMILES: C#Cc1c(C(F)(F)F)ccc2cc(O)cc(-c3ccc4c(N5CC6CCC(C5)N6)nc(OCC5(CN6CCOCC6)CC5)nc4c3F)c12 ; C#Cc1c(OC)ccc2cc(O)cc(-c3ccc4c(N5CC6CCC(C5)N6)nc(OCC5(CN6CCOCC6)CC5)nc4c3F)c12 ; C#Cc1c(C)ccc2cc(O)cc(-c3ccc4c(N5CC6CCC(C5)N6)nc(OCC5(CN6CCOCC6)CC5)nc4c3F)c12 ; C#Cc1c(Cl)ccc2cc(O)cc(-c3ccc4c(N5CC6CCC(C5)N6)nc(OCC5(CN6CCOCC6)CC5)nc4c3F)c12]

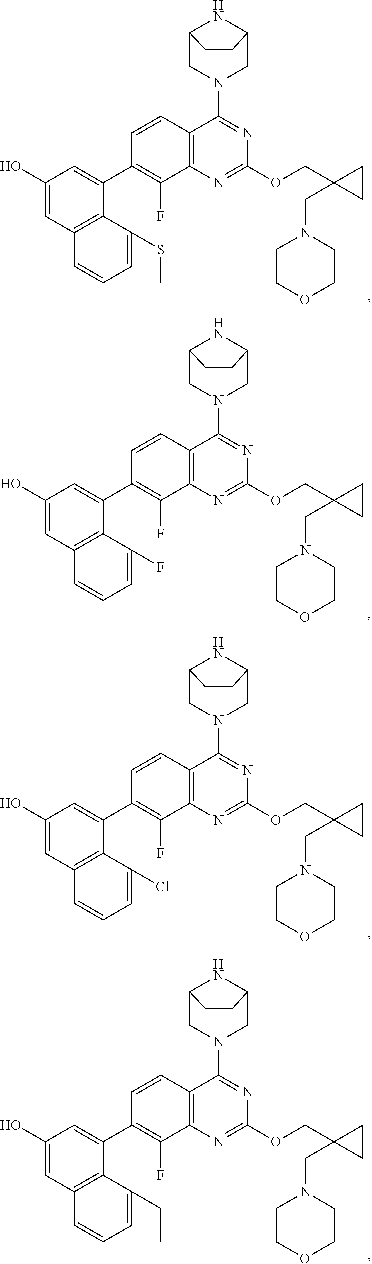 [SMILES: CSc1cccc2cc(O)cc(-c3ccc4c(N5CC6CCC(C5)N6)nc(OCC5(CN6CCOCC6)CC5)nc4c3F)c12 ; Oc1cc(-c2ccc3c(N4CC5CCC(C4)N5)nc(OCC4(CN5CCOCC5)CC4)nc3c2F)c2c(F)cccc2c1 ; Oc1cc(-c2ccc3c(N4CC5CCC(C4)N5)nc(OCC4(CN5CCOCC5)CC4)nc3c2F)c2c(Cl)cccc2c1 ; CCc1cccc2cc(O)cc(-c3ccc4c(N5CC6CCC(C5)N6)nc(OCC5(CN6CCOCC6)CC5)nc4c3F)c12]

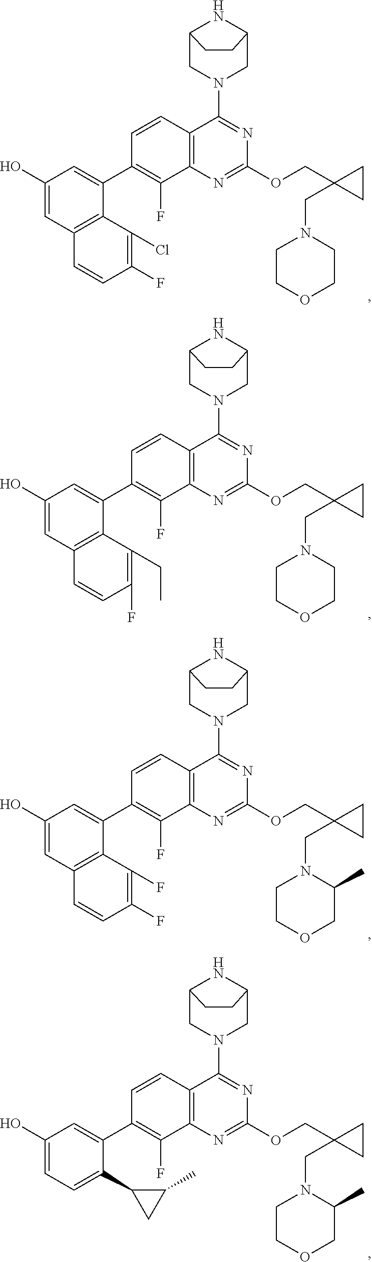 [SMILES: Oc1cc(-c2ccc3c(N4CC5CCC(C4)N5)nc(OCC4(CN5CCOCC5)CC4)nc3c2F)c2c(Cl)c(F)ccc2c1 ; CCc1c(F)ccc2cc(O)cc(-c3ccc4c(N5CC6CCC(C5)N6)nc(OCC5(CN6CCOCC6)CC5)nc4c3F)c12 ; C[C@H]1COCCN1CC1(COc2nc(N3CC4CCC(C3)N4)c3ccc(-c4cc(O)cc5ccc(F)c(F)c45)c(F)c3n2)CC1 ; C[C@@H]1C[C@H]1c1ccc(O)cc1-c1ccc2c(N3CC4CCC(C3)N4)nc(OCC3(CN4CCOC[C@@H]4C)CC3)nc2c1F]

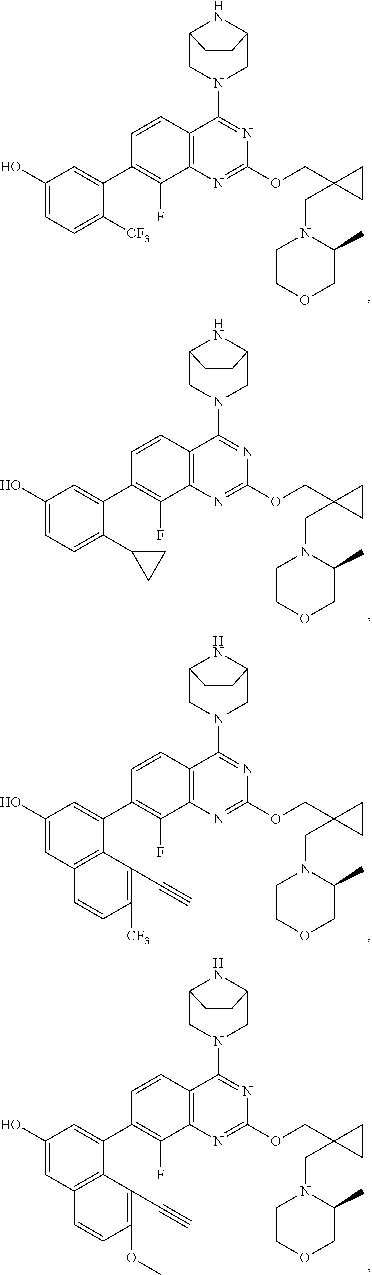 [SMILES: C[C@H]1COCCN1CC1(COc2nc(N3CC4CCC(C3)N4)c3ccc(-c4cc(O)ccc4C(F)(F)F)c(F)c3n2)CC1 ; C[C@H]1COCCN1CC1(COc2nc(N3CC4CCC(C3)N4)c3ccc(-c4cc(O)ccc4C4CC4)c(F)c3n2)CC1 ; C#Cc1c(C(F)(F)F)ccc2cc(O)cc(-c3ccc4c(N5CC6CCC(C5)N6)nc(OCC5(CN6CCOC[C@@H]6C)CC5)nc4c3F)c12 ; C#Cc1c(OC)ccc2cc(O)cc(-c3ccc4c(N5CC6CCC(C5)N6)nc(OCC5(CN6CCOC[C@@H]6C)CC5)nc4c3F)c12]

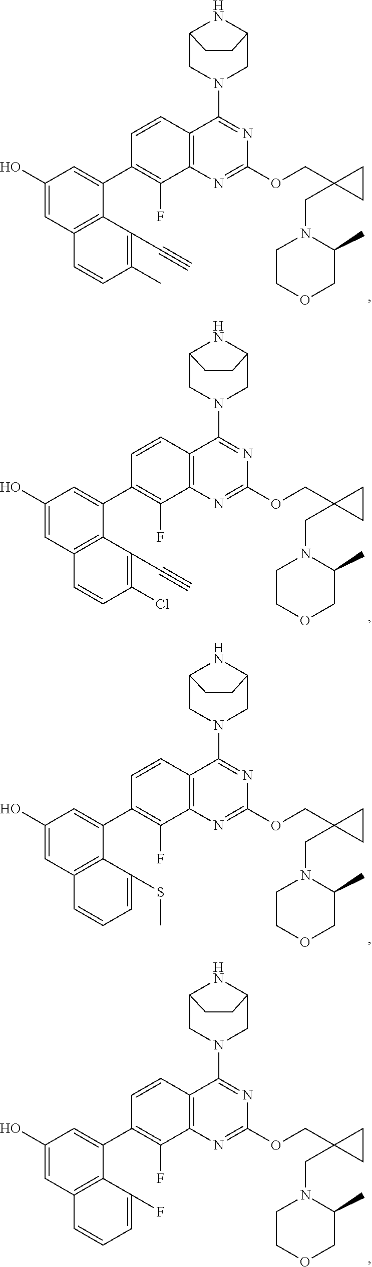 [SMILES: C#Cc1c(C)ccc2cc(O)cc(-c3ccc4c(N5CC6CCC(C5)N6)nc(OCC5(CN6CCOC[C@@H]6C)CC5)nc4c3F)c12 ; C#Cc1c(Cl)ccc2cc(O)cc(-c3ccc4c(N5CC6CCC(C5)N6)nc(OCC5(CN6CCOC[C@@H]6C)CC5)nc4c3F)c12 ; CSc1cccc2cc(O)cc(-c3ccc4c(N5CC6CCC(C5)N6)nc(OCC5(CN6CCOC[C@@H]6C)CC5)nc4c3F)c12 ; C[C@H]1COCCN1CC1(COc2nc(N3CC4CCC(C3)N4)c3ccc(-c4cc(O)cc5cccc(F)c45)c(F)c3n2)CC1]

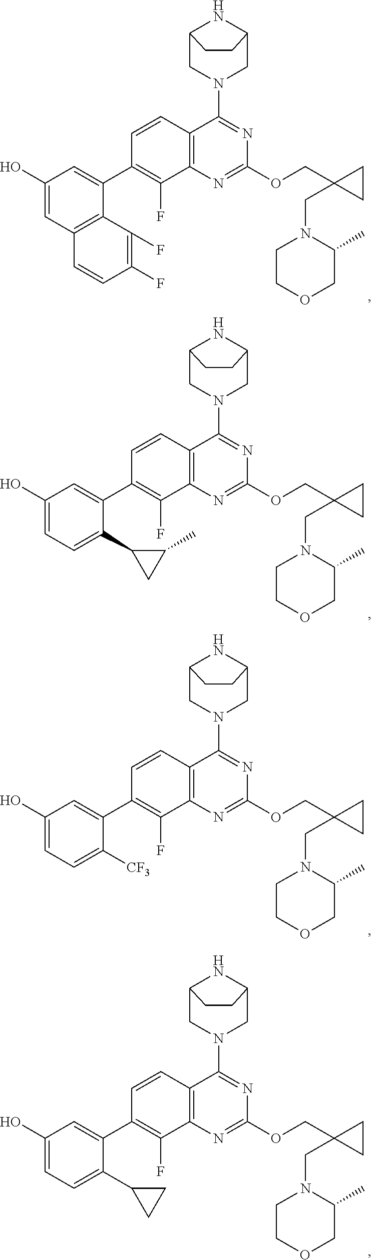 [SMILES: C[C@@H]1COCCN1CC1(COc2nc(N3CC4CCC(C3)N4)c3ccc(-c4cc(O)cc5ccc(F)c(F)c45)c(F)c3n2)CC1 ; C[C@@H]1C[C@H]1c1ccc(O)cc1-c1ccc2c(N3CC4CCC(C3)N4)nc(OCC3(CN4CCOC[C@H]4C)CC3)nc2c1F ; C[C@@H]1COCCN1CC1(COc2nc(N3CC4CCC(C3)N4)c3ccc(-c4cc(O)ccc4C(F)(F)F)c(F)c3n2)CC1 ; C[C@@H]1COCCN1CC1(COc2nc(N3CC4CCC(C3)N4)c3ccc(-c4cc(O)ccc4C4CC4)c(F)c3n2)CC1]

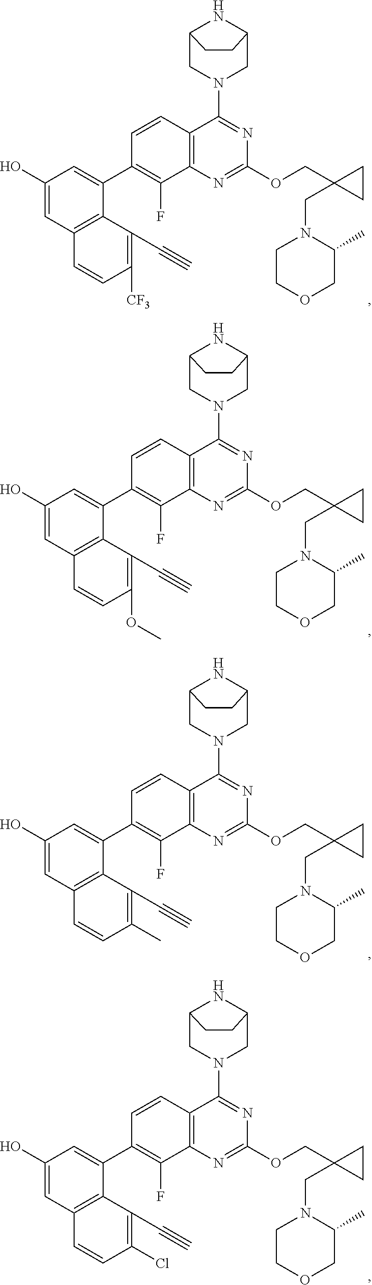 [SMILES: C#Cc1c(C(F)(F)F)ccc2cc(O)cc(-c3ccc4c(N5CC6CCC(C5)N6)nc(OCC5(CN6CCOC[C@H]6C)CC5)nc4c3F)c12 ; C#Cc1c(OC)ccc2cc(O)cc(-c3ccc4c(N5CC6CCC(C5)N6)nc(OCC5(CN6CCOC[C@H]6C)CC5)nc4c3F)c12 ; C#Cc1c(C)ccc2cc(O)cc(-c3ccc4c(N5CC6CCC(C5)N6)nc(OCC5(CN6CCOC[C@H]6C)CC5)nc4c3F)c12 ; C#Cc1c(Cl)ccc2cc(O)cc(-c3ccc4c(N5CC6CCC(C5)N6)nc(OCC5(CN6CCOC[C@H]6C)CC5)nc4c3F)c12]

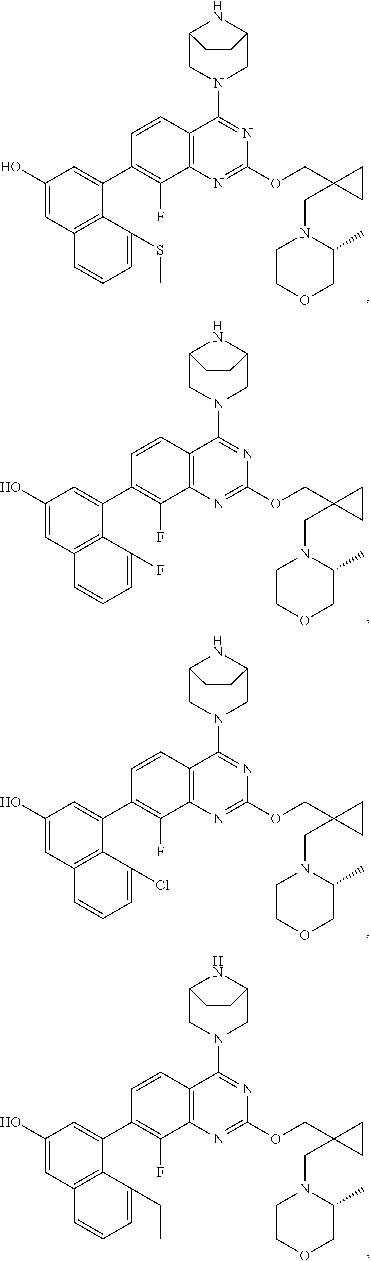 [SMILES: CSc1cccc2cc(O)cc(-c3ccc4c(N5CC6CCC(C5)N6)nc(OCC5(CN6CCOC[C@H]6C)CC5)nc4c3F)c12 ; C[C@@H]1COCCN1CC1(COc2nc(N3CC4CCC(C3)N4)c3ccc(-c4cc(O)cc5cccc(F)c45)c(F)c3n2)CC1 ; C[C@@H]1COCCN1CC1(COc2nc(N3CC4CCC(C3)N4)c3ccc(-c4cc(O)cc5cccc(Cl)c45)c(F)c3n2)CC1 ; CCc1cccc2cc(O)cc(-c3ccc4c(N5CC6CCC(C5)N6)nc(OCC5(CN6CCOC[C@H]6C)CC5)nc4c3F)c12]

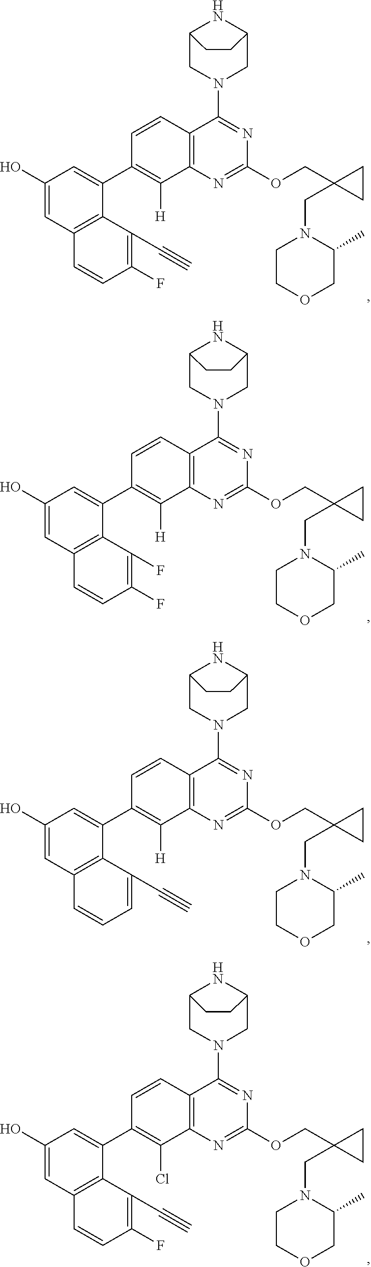 [SMILES: C#Cc1c(F)ccc2cc(O)cc(-c3ccc4c(N5CC6CCC(C5)N6)nc(OCC5(CN6CCOC[C@H]6C)CC5)nc4c3)c12 ; C[C@@H]1COCCN1CC1(COc2nc(N3CC4CCC(C3)N4)c3ccc(-c4cc(O)cc5ccc(F)c(F)c45)cc3n2)CC1 ; C#Cc1cccc2cc(O)cc(-c3ccc4c(N5CC6CCC(C5)N6)nc(OCC5(CN6CCOC[C@H]6C)CC5)nc4c3)c12 ; C#Cc1c(F)ccc2cc(O)cc(-c3ccc4c(N5CC6CCC(C5)N6)nc(OCC5(CN6CCOC[C@H]6C)CC5)nc4c3Cl)c12]

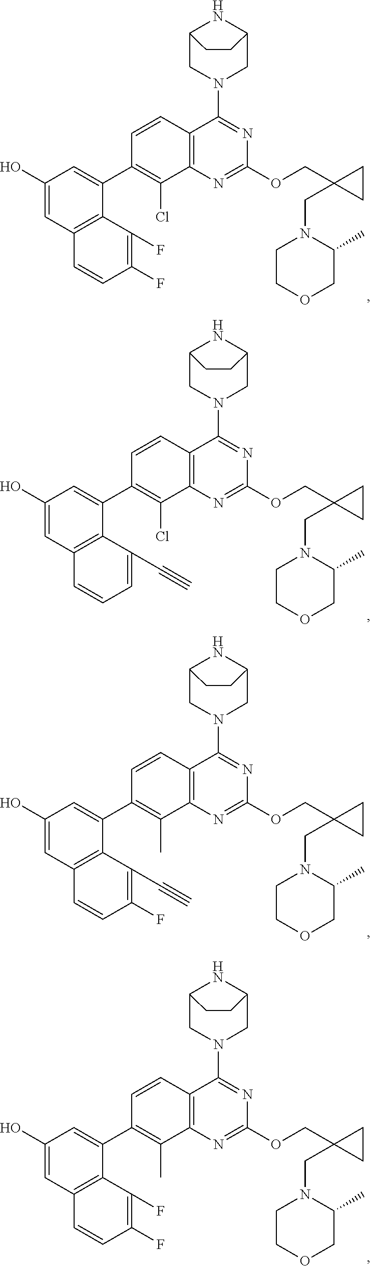 [SMILES: C[C@@H]1COCCN1CC1(COc2nc(N3CC4CCC(C3)N4)c3ccc(-c4cc(O)cc5ccc(F)c(F)c45)c(Cl)c3n2)CC1 ; C#Cc1cccc2cc(O)cc(-c3ccc4c(N5CC6CCC(C5)N6)nc(OCC5(CN6CCOC[C@H]6C)CC5)nc4c3Cl)c12 ; C#Cc1c(F)ccc2cc(O)cc(-c3ccc4c(N5CC6CCC(C5)N6)nc(OCC5(CN6CCOC[C@H]6C)CC5)nc4c3C)c12 ; Cc1c(-c2cc(O)cc3ccc(F)c(F)c23)ccc2c(N3CC4CCC(C3)N4)nc(OCC3(CN4CCOC[C@H]4C)CC3)nc12]

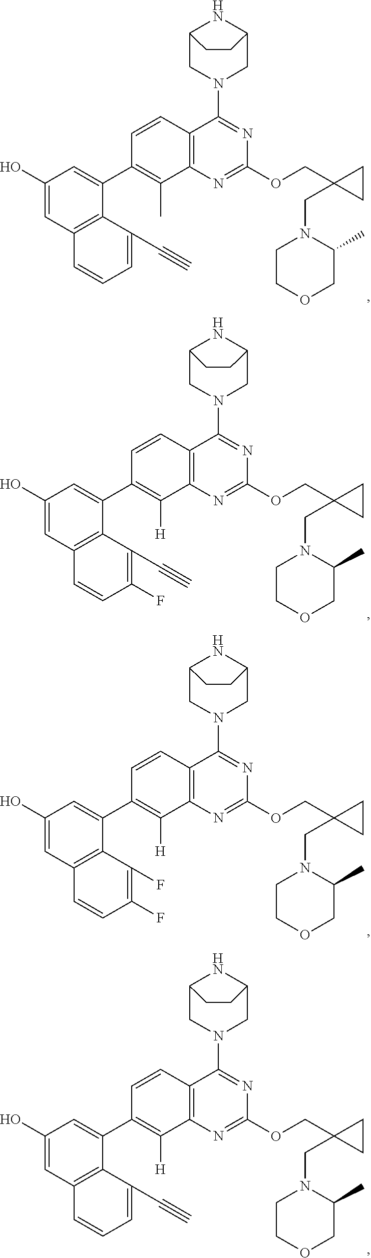 [SMILES: C#Cc1cccc2cc(O)cc(-c3ccc4c(N5CC6CCC(C5)N6)nc(OCC5(CN6CCOC[C@H]6C)CC5)nc4c3C)c12 ; C#Cc1c(F)ccc2cc(O)cc(-c3ccc4c(N5CC6CCC(C5)N6)nc(OCC5(CN6CCOC[C@@H]6C)CC5)nc4c3)c12 ; C[C@H]1COCCN1CC1(COc2nc(N3CC4CCC(C3)N4)c3ccc(-c4cc(O)cc5ccc(F)c(F)c45)cc3n2)CC1 ; C#Cc1cccc2cc(O)cc(-c3ccc4c(N5CC6CCC(C5)N6)nc(OCC5(CN6CCOC[C@@H]6C)CC5)nc4c3)c12]

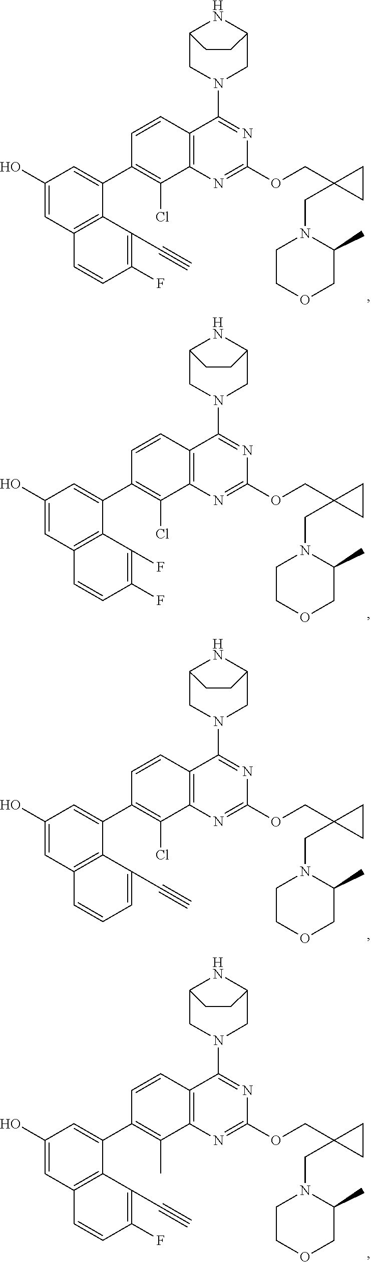 [SMILES: C#Cc1c(F)ccc2cc(O)cc(-c3ccc4c(N5CC6CCC(C5)N6)nc(OCC5(CN6CCOC[C@@H]6C)CC5)nc4c3Cl)c12 ; C[C@H]1COCCN1CC1(COc2nc(N3CC4CCC(C3)N4)c3ccc(-c4cc(O)cc5ccc(F)c(F)c45)c(Cl)c3n2)CC1 ; C#Cc1cccc2cc(O)cc(-c3ccc4c(N5CC6CCC(C5)N6)nc(OCC5(CN6CCOC[C@@H]6C)CC5)nc4c3Cl)c12 ; C#Cc1c(F)ccc2cc(O)cc(-c3ccc4c(N5CC6CCC(C5)N6)nc(OCC5(CN6CCOC[C@@H]6C)CC5)nc4c3C)c12]

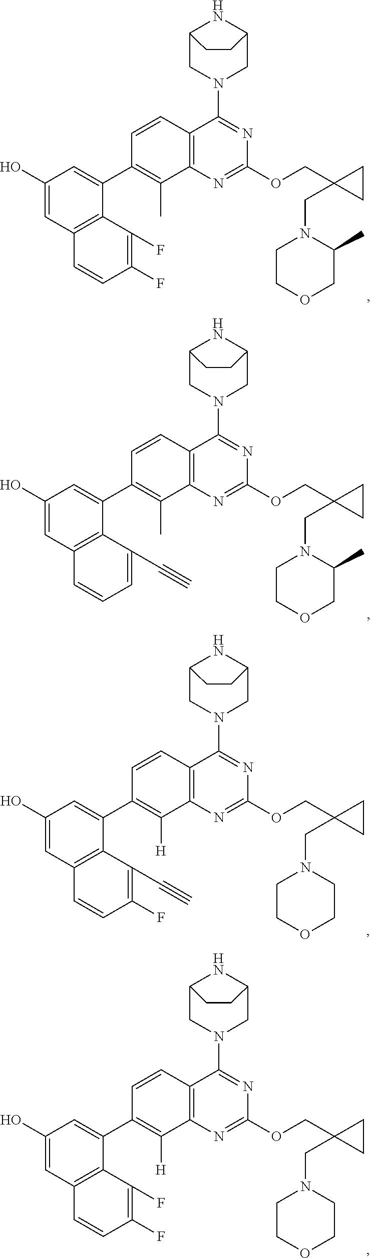 [SMILES: Cc1c(-c2cc(O)cc3ccc(F)c(F)c23)ccc2c(N3CC4CCC(C3)N4)nc(OCC3(CN4CCOC[C@@H]4C)CC3)nc12 ; C#Cc1cccc2cc(O)cc(-c3ccc4c(N5CC6CCC(C5)N6)nc(OCC5(CN6CCOC[C@@H]6C)CC5)nc4c3C)c12 ; C#Cc1c(F)ccc2cc(O)cc(-c3ccc4c(N5CC6CCC(C5)N6)nc(OCC5(CN6CCOCC6)CC5)nc4c3)c12 ; Oc1cc(-c2ccc3c(N4CC5CCC(C4)N5)nc(OCC4(CN5CCOCC5)CC4)nc3c2)c2c(F)c(F)ccc2c1]

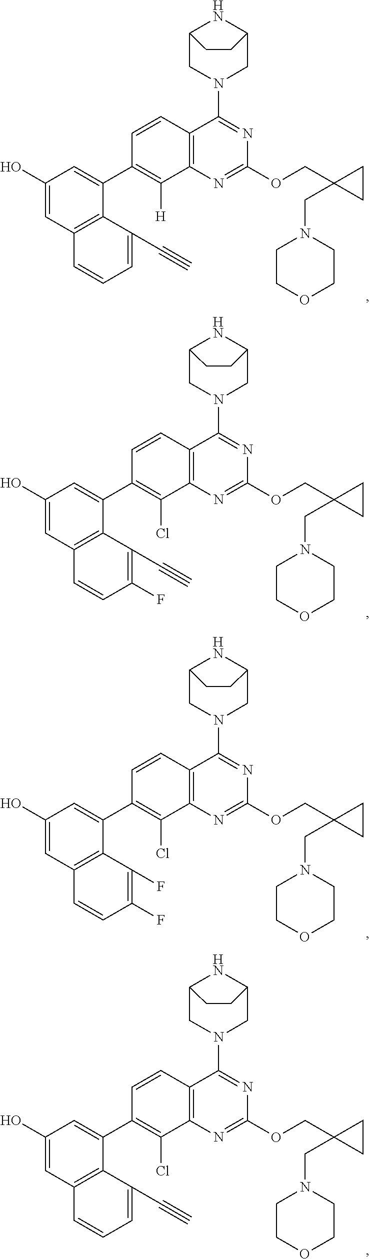 [SMILES: C#Cc1cccc2cc(O)cc(-c3ccc4c(N5CC6CCC(C5)N6)nc(OCC5(CN6CCOCC6)CC5)nc4c3)c12 ; C#Cc1c(F)ccc2cc(O)cc(-c3ccc4c(N5CC6CCC(C5)N6)nc(OCC5(CN6CCOCC6)CC5)nc4c3Cl)c12 ; Oc1cc(-c2ccc3c(N4CC5CCC(C4)N5)nc(OCC4(CN5CCOCC5)CC4)nc3c2Cl)c2c(F)c(F)ccc2c1 ; C#Cc1cccc2cc(O)cc(-c3ccc4c(N5CC6CCC(C5)N6)nc(OCC5(CN6CCOCC6)CC5)nc4c3Cl)c12]

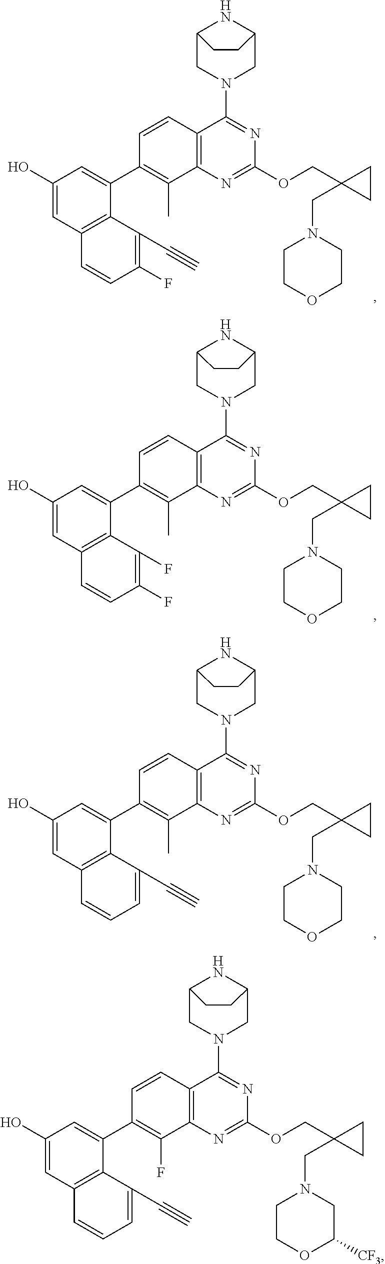 [SMILES: C#Cc1c(F)ccc2cc(O)cc(-c3ccc4c(N5CC6CCC(C5)N6)nc(OCC5(CN6CCOCC6)CC5)nc4c3C)c12 ; Cc1c(-c2cc(O)cc3ccc(F)c(F)c23)ccc2c(N3CC4CCC(C3)N4)nc(OCC3(CN4CCOCC4)CC3)nc12 ; C#Cc1cccc2cc(O)cc(-c3ccc4c(N5CC6CCC(C5)N6)nc(OCC5(CN6CCOCC6)CC5)nc4c3C)c12 ; C#Cc1cccc2cc(O)cc(-c3ccc4c(N5CC6CCC(C5)N6)nc(OCC5(CN6CCO[C@@H](C(F)(F)F)C6)CC5)nc4c3F)c12]

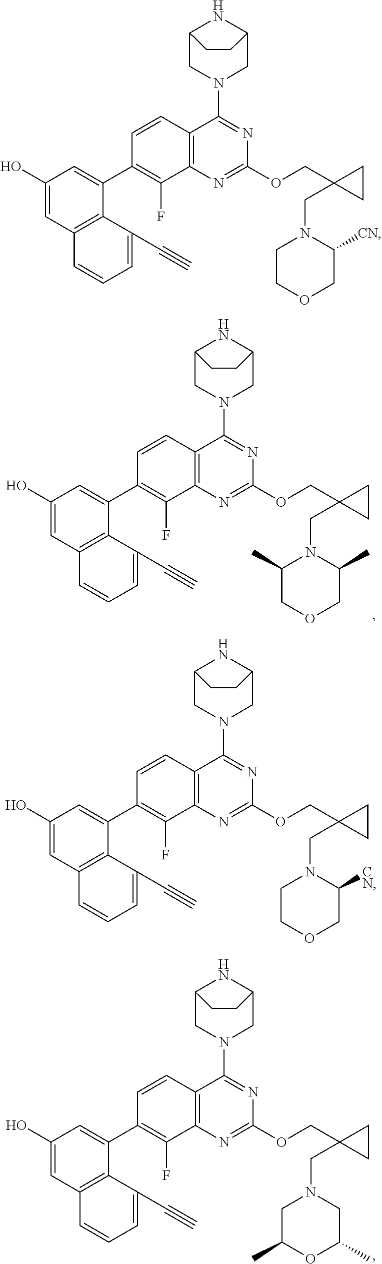 [SMILES: C#Cc1cccc2cc(O)cc(-c3ccc4c(N5CC6CCC(C5)N6)nc(OCC5(CN6CCOC[C@H]6C#N)CC5)nc4c3F)c12 ; C#Cc1cccc2cc(O)cc(-c3ccc4c(N5CC6CCC(C5)N6)nc(OCC5(CN6[C@H](C)COC[C@@H]6C)CC5)nc4c3F)c12 ; C#Cc1cccc2cc(O)cc(-c3ccc4c(N5CC6CCC(C5)N6)nc(OCC5(CN6CCOC[C@@H]6C#N)CC5)nc4c3F)c12 ; C#Cc1cccc2cc(O)cc(-c3ccc4c(N5CC6CCC(C5)N6)nc(OCC5(CN6C[C@H](C)O[C@@H](C)C6)CC5)nc4c3F)c12]

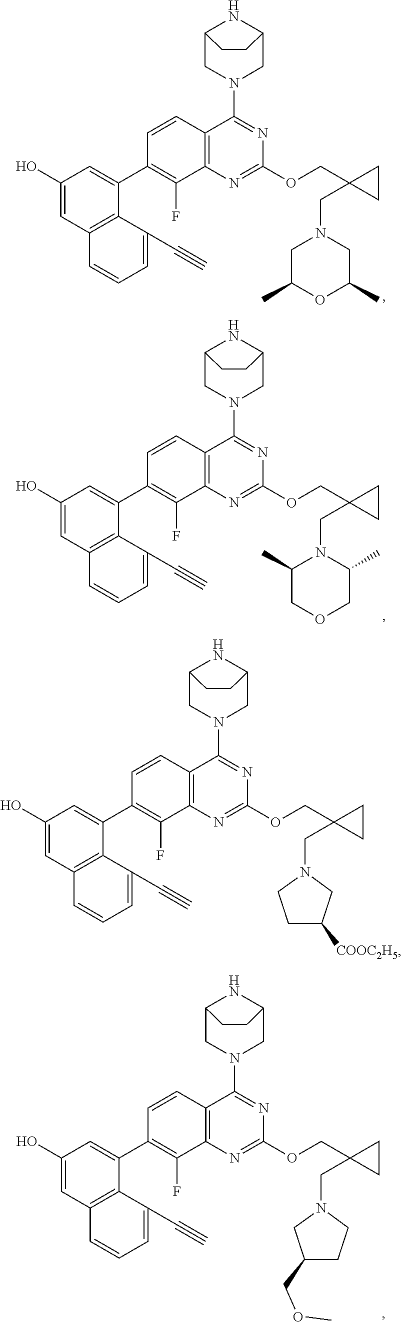

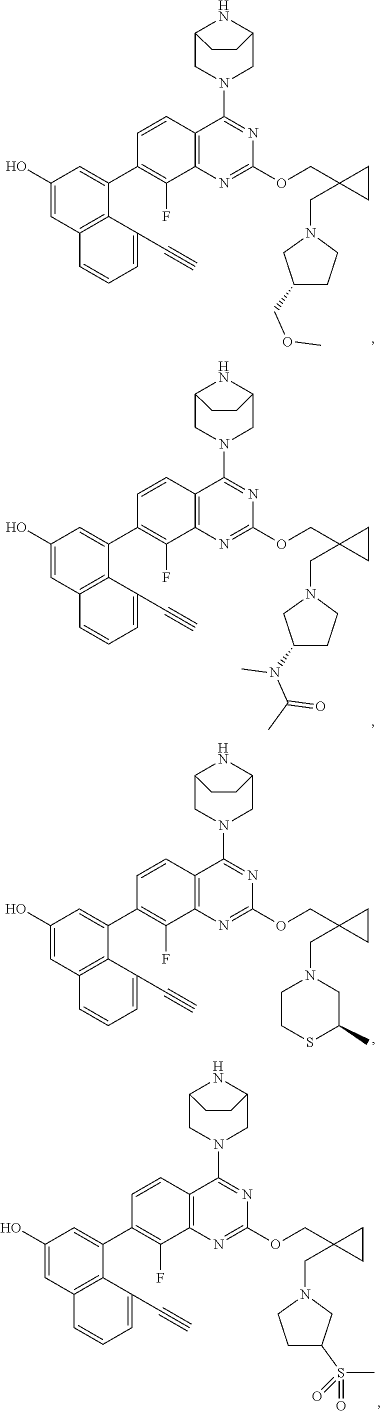 [SMILES: C#Cc1cccc2cc(O)cc(-c3ccc4c(N5CC6CCC(C5)N6)nc(OCC5(CN6CC[C@H](COC)C6)CC5)nc4c3F)c12 ; C#Cc1cccc2cc(O)cc(-c3ccc4c(N5CC6CCC(C5)N6)nc(OCC5(CN6CC[C@H](N(C)C(C)=O)C6)CC5)nc4c3F)c12 ; C#Cc1cccc2cc(O)cc(-c3ccc4c(N5CC6CCC(C5)N6)nc(OCC5(CN6CCS[C@H](C)C6)CC5)nc4c3F)c12 ; C#Cc1cccc2cc(O)cc(-c3ccc4c(N5CC6CCC(C5)N6)nc(OCC5(CN6CCC(S(C)(=O)=O)C6)CC5)nc4c3F)c12]

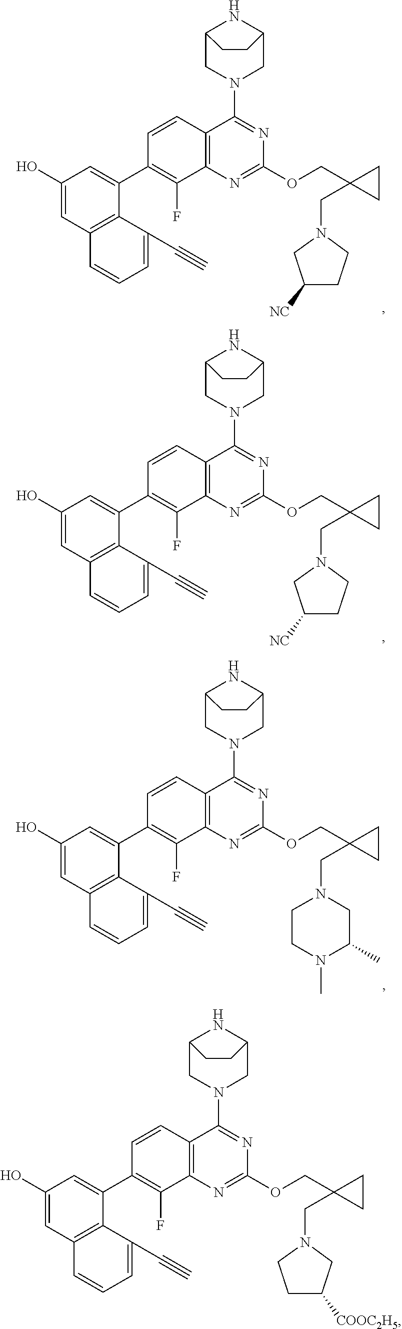

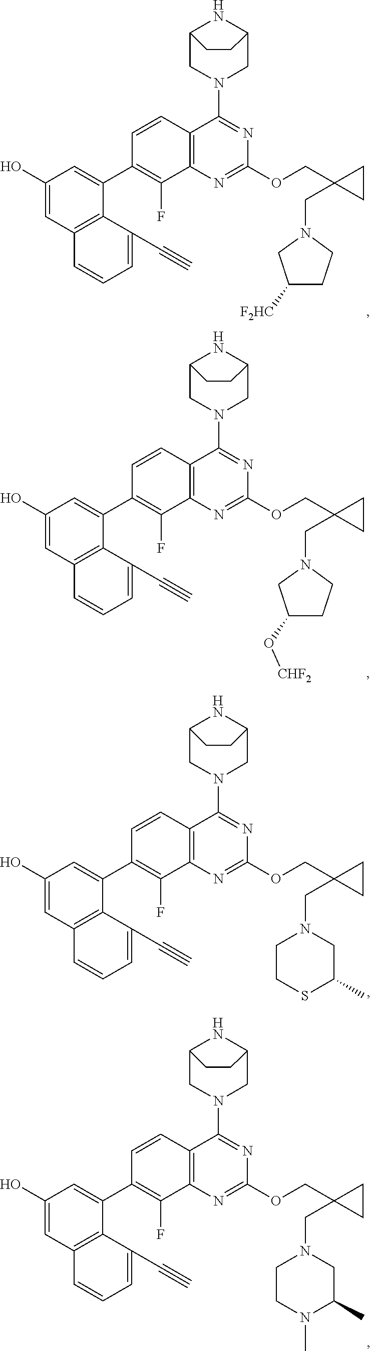 [SMILES: C#Cc1cccc2cc(O)cc(-c3ccc4c(N5CC6CCC(C5)N6)nc(OCC5(CN6CC[C@H](C(F)F)C6)CC5)nc4c3F)c12 ; C#Cc1cccc2cc(O)cc(-c3ccc4c(N5CC6CCC(C5)N6)nc(OCC5(CN6CC[C@H](OC(F)F)C6)CC5)nc4c3F)c12 ; C#Cc1cccc2cc(O)cc(-c3ccc4c(N5CC6CCC(C5)N6)nc(OCC5(CN6CCS[C@@H](C)C6)CC5)nc4c3F)c12 ; C#Cc1cccc2cc(O)cc(-c3ccc4c(N5CC6CCC(C5)N6)nc(OCC5(CN6CCN(C)[C@H](C)C6)CC5)nc4c3F)c12]

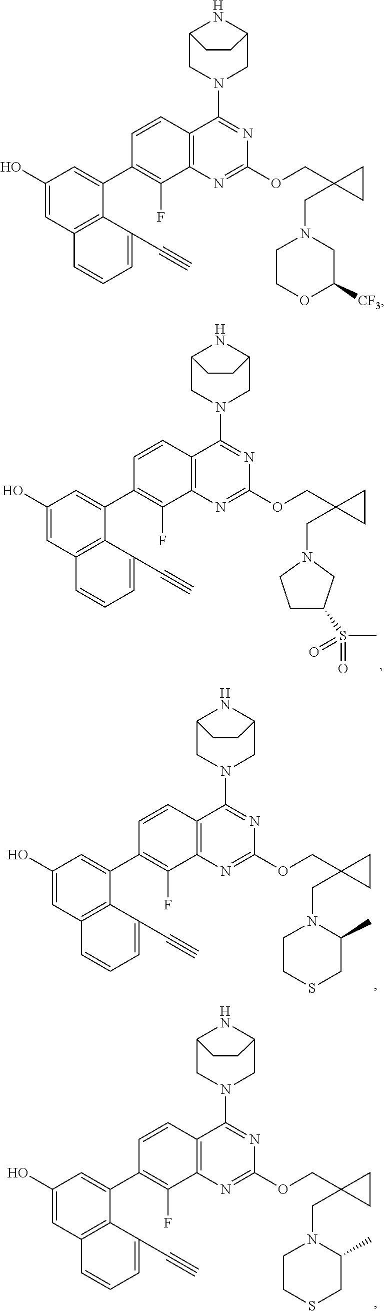 [SMILES: C#Cc1cccc2cc(O)cc(-c3ccc4c(N5CC6CCC(C5)N6)nc(OCC5(CN6CCO[C@H](C(F)(F)F)C6)CC5)nc4c3F)c12 ; C#Cc1cccc2cc(O)cc(-c3ccc4c(N5CC6CCC(C5)N6)nc(OCC5(CN6CC[C@@H](S(C)(=O)=O)C6)CC5)nc4c3F)c12 ; C#Cc1cccc2cc(O)cc(-c3ccc4c(N5CC6CCC(C5)N6)nc(OCC5(CN6CCSC[C@@H]6C)CC5)nc4c3F)c12 ; C#Cc1cccc2cc(O)cc(-c3ccc4c(N5CC6CCC(C5)N6)nc(OCC5(CN6CCSC[C@H]6C)CC5)nc4c3F)c12]

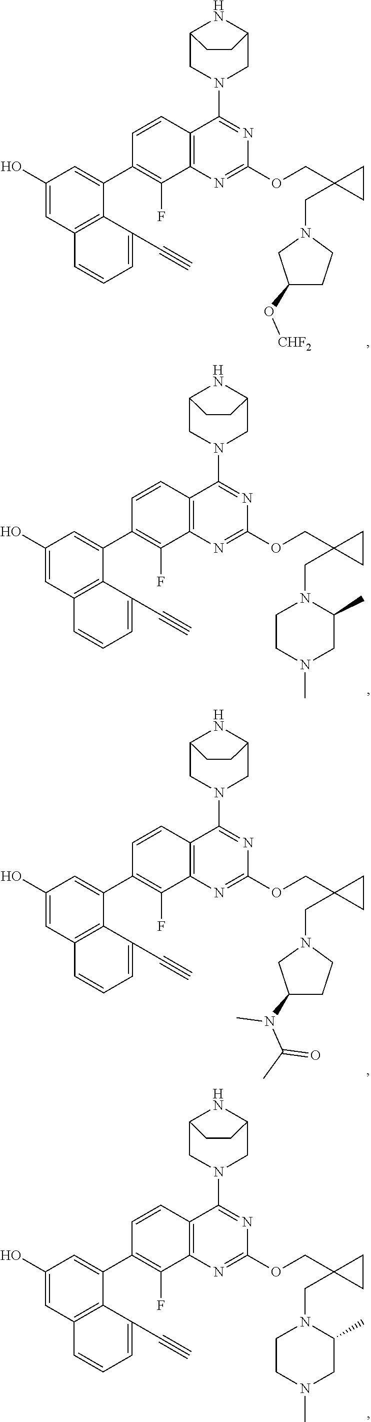

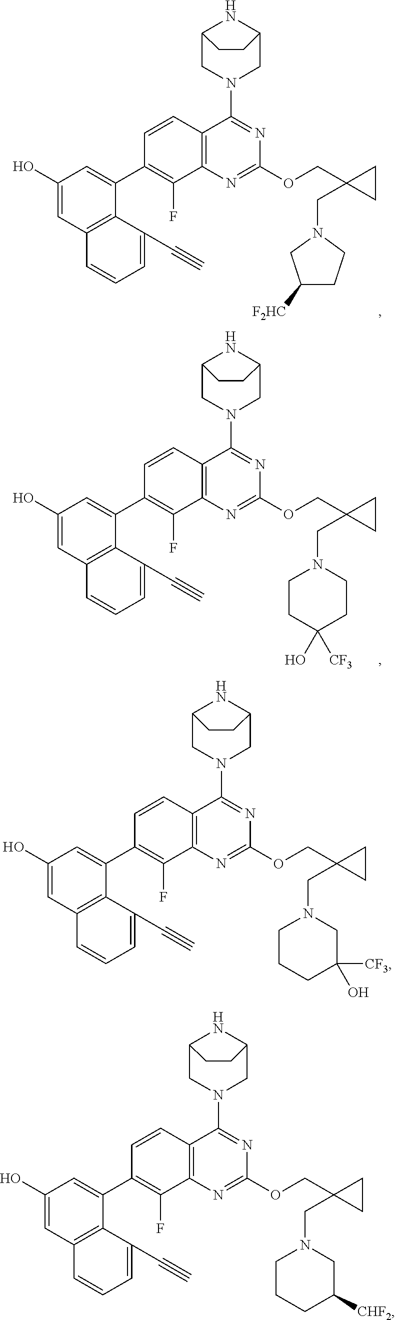 [SMILES: C#Cc1cccc2cc(O)cc(-c3ccc4c(N5CC6CCC(C5)N6)nc(OCC5(CN6CC[C@@H](C(F)F)C6)CC5)nc4c3F)c12 ; C#Cc1cccc2cc(O)cc(-c3ccc4c(N5CC6CCC(C5)N6)nc(OCC5(CN6CCC(O)(C(F)(F)F)CC6)CC5)nc4c3F)c12 ; C#Cc1cccc2cc(O)cc(-c3ccc4c(N5CC6CCC(C5)N6)nc(OCC5(CN6CCCC(O)(C(F)(F)F)C6)CC5)nc4c3F)c12 ; C#Cc1cccc2cc(O)cc(-c3ccc4c(N5CC6CCC(C5)N6)nc(OCC5(CN6CCC[C@H](C(F)F)C6)CC5)nc4c3F)c12]

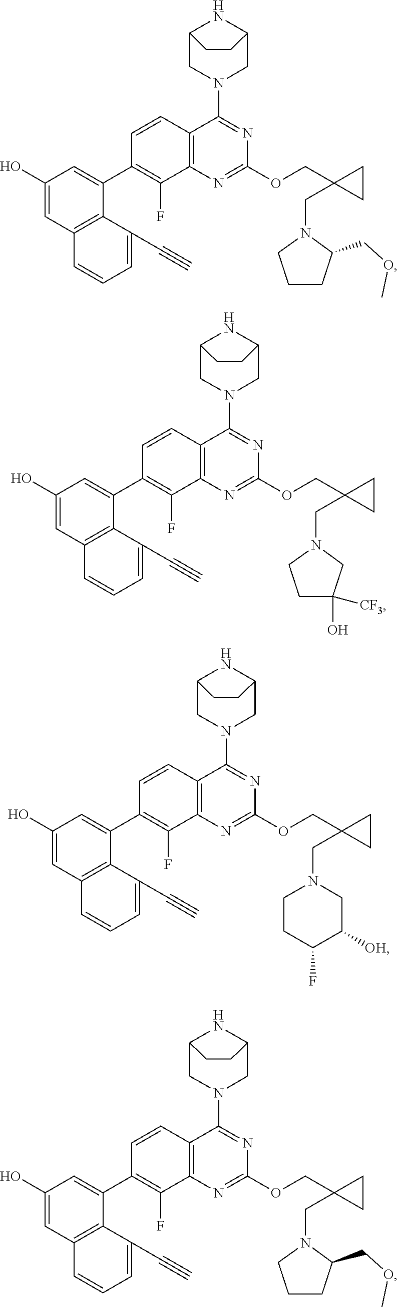 [SMILES: C#Cc1cccc2cc(O)cc(-c3ccc4c(N5CC6CCC(C5)N6)nc(OCC5(CN6CCC[C@H]6COC)CC5)nc4c3F)c12 ; C#Cc1cccc2cc(O)cc(-c3ccc4c(N5CC6CCC(C5)N6)nc(OCC5(CN6CCC(O)(C(F)(F)F)C6)CC5)nc4c3F)c12 ; C#Cc1cccc2cc(O)cc(-c3ccc4c(N5CC6CCC(C5)N6)nc(OCC5(CN6CC[C@@H](F)[C@@H](O)C6)CC5)nc4c3F)c12 ; C#Cc1cccc2cc(O)cc(-c3ccc4c(N5CC6CCC(C5)N6)nc(OCC5(CN6CCC[C@@H]6COC)CC5)nc4c3F)c12]

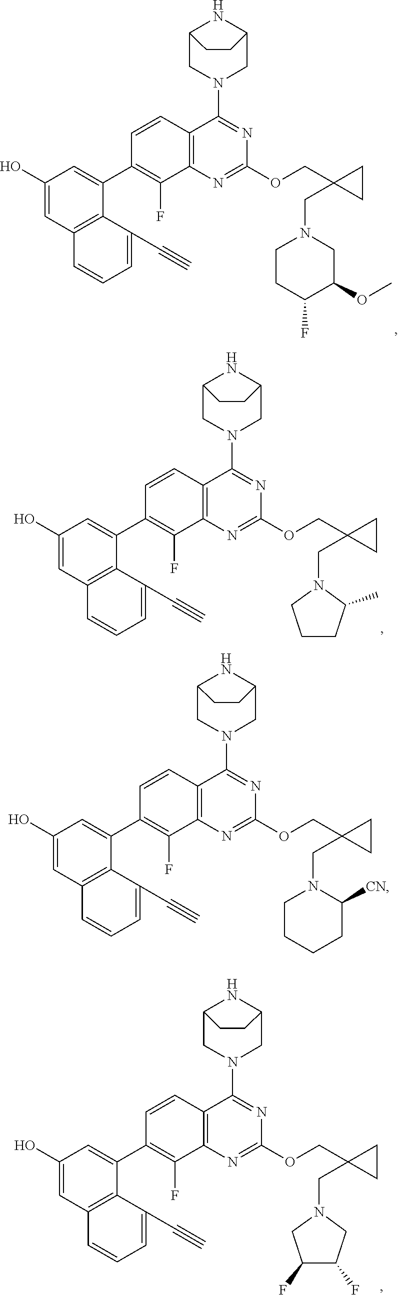 [SMILES: C#Cc1cccc2cc(O)cc(-c3ccc4c(N5CC6CCC(C5)N6)nc(OCC5(CN6CC[C@@H](F)[C@H](OC)C6)CC5)nc4c3F)c12 ; C#Cc1cccc2cc(O)cc(-c3ccc4c(N5CC6CCC(C5)N6)nc(OCC5(CN6CCC[C@H]6C)CC5)nc4c3F)c12 ; C#Cc1cccc2cc(O)cc(-c3ccc4c(N5CC6CCC(C5)N6)nc(OCC5(CN6CCCC[C@@H]6C#N)CC5)nc4c3F)c12 ; C#Cc1cccc2cc(O)cc(-c3ccc4c(N5CC6CCC(C5)N6)nc(OCC5(CN6C[C@H](F)[C@@H](F)C6)CC5)nc4c3F)c12]

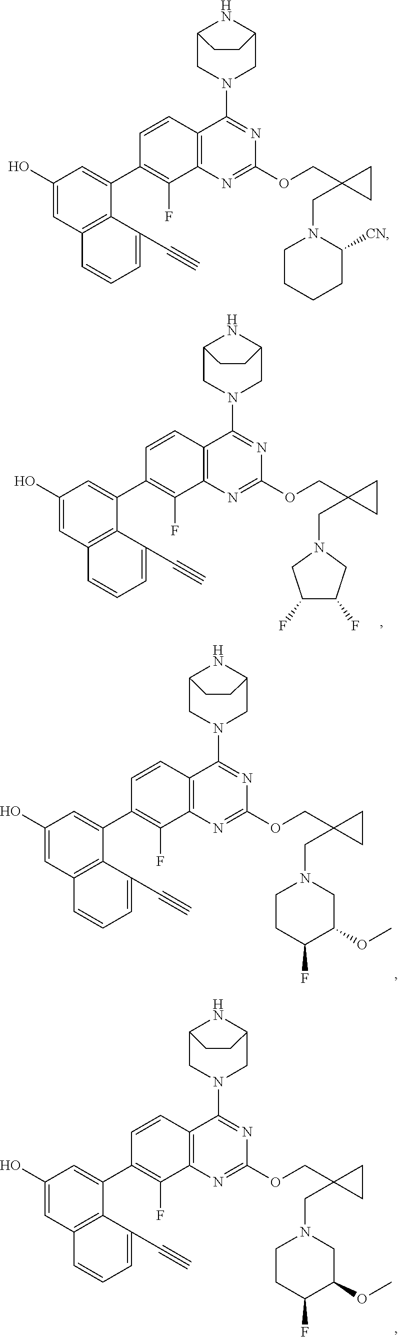 [SMILES: C#Cc1cccc2cc(O)cc(-c3ccc4c(N5CC6CCC(C5)N6)nc(OCC5(CN6CCCC[C@H]6C#N)CC5)nc4c3F)c12 ; C#Cc1cccc2cc(O)cc(-c3ccc4c(N5CC6CCC(C5)N6)nc(OCC5(CN6C[C@@H](F)[C@@H](F)C6)CC5)nc4c3F)c12 ; C#Cc1cccc2cc(O)cc(-c3ccc4c(N5CC6CCC(C5)N6)nc(OCC5(CN6CC[C@H](F)[C@@H](OC)C6)CC5)nc4c3F)c12 ; C#Cc1cccc2cc(O)cc(-c3ccc4c(N5CC6CCC(C5)N6)nc(OCC5(CN6CC[C@H](F)[C@H](OC)C6)CC5)nc4c3F)c12]

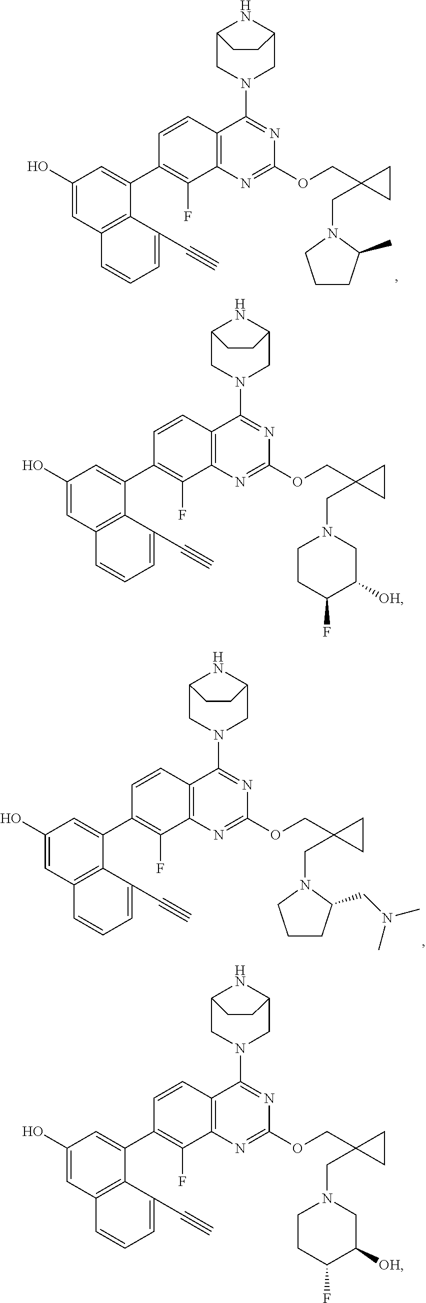 [SMILES: C#Cc1cccc2cc(O)cc(-c3ccc4c(N5CC6CCC(C5)N6)nc(OCC5(CN6CCC[C@@H]6C)CC5)nc4c3F)c12 ; C#Cc1cccc2cc(O)cc(-c3ccc4c(N5CC6CCC(C5)N6)nc(OCC5(CN6CC[C@H](F)[C@@H](O)C6)CC5)nc4c3F)c12 ; C#Cc1cccc2cc(O)cc(-c3ccc4c(N5CC6CCC(C5)N6)nc(OCC5(CN6CCC[C@H]6CN(C)C)CC5)nc4c3F)c12 ; C#Cc1cccc2cc(O)cc(-c3ccc4c(N5CC6CCC(C5)N6)nc(OCC5(CN6CC[C@@H](F)[C@H](O)C6)CC5)nc4c3F)c12]

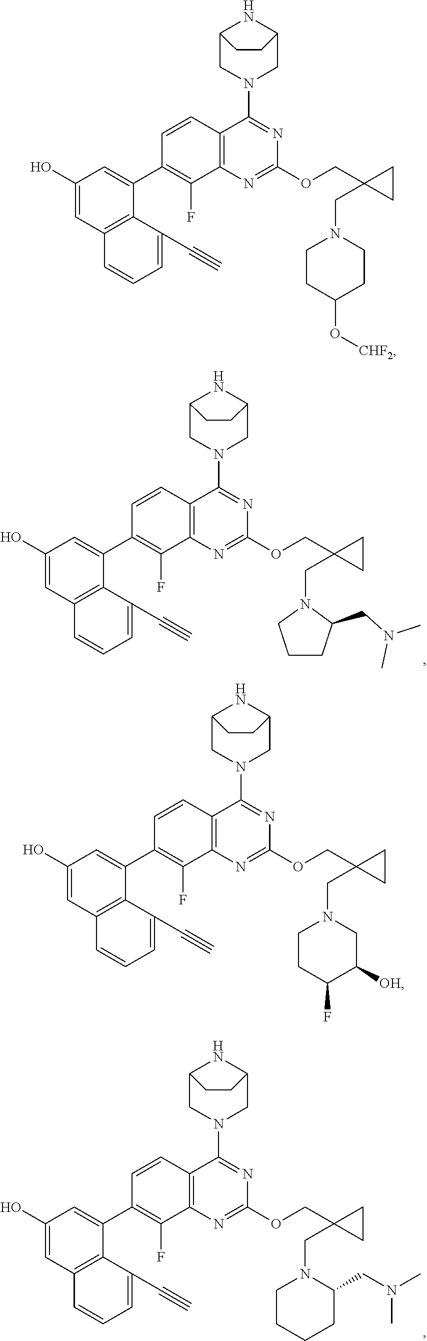 [SMILES: C#Cc1cccc2cc(O)cc(-c3ccc4c(N5CC6CCC(C5)N6)nc(OCC5(CN6CCC(OC(F)F)CC6)CC5)nc4c3F)c12 ; C#Cc1cccc2cc(O)cc(-c3ccc4c(N5CC6CCC(C5)N6)nc(OCC5(CN6CCC[C@@H]6CN(C)C)CC5)nc4c3F)c12 ; C#Cc1cccc2cc(O)cc(-c3ccc4c(N5CC6CCC(C5)N6)nc(OCC5(CN6CC[C@H](F)[C@H](O)C6)CC5)nc4c3F)c12 ; C#Cc1cccc2cc(O)cc(-c3ccc4c(N5CC6CCC(C5)N6)nc(OCC5(CN6CCCC[C@H]6CN(C)C)CC5)nc4c3F)c12]

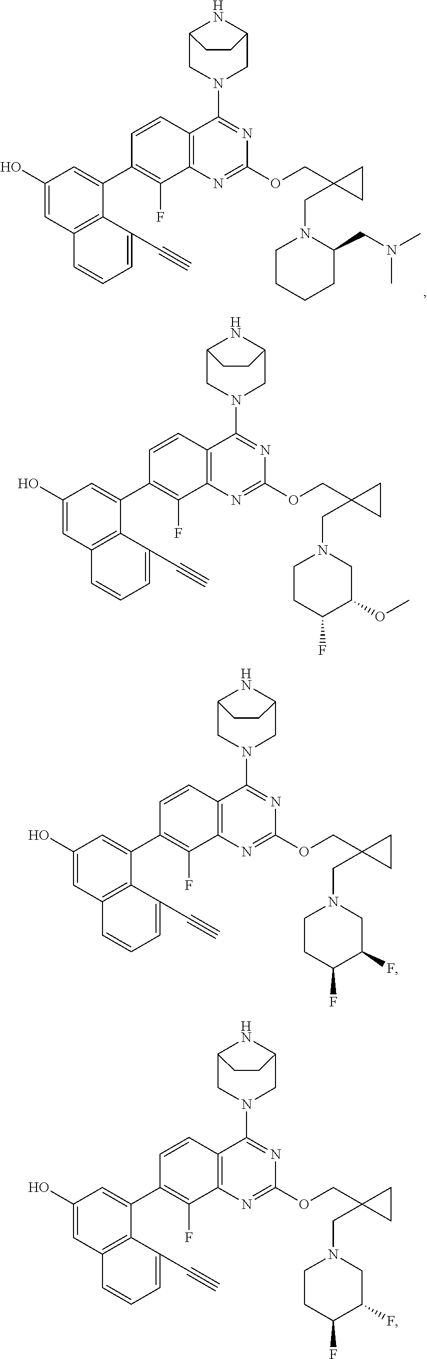 [SMILES: C#Cc1cccc2cc(O)cc(-c3ccc4c(N5CC6CCC(C5)N6)nc(OCC5(CN6CCCC[C@@H]6CN(C)C)CC5)nc4c3F)c12 ; C#Cc1cccc2cc(O)cc(-c3ccc4c(N5CC6CCC(C5)N6)nc(OCC5(CN6CC[C@@H](F)[C@@H](OC)C6)CC5)nc4c3F)c12 ; C#Cc1cccc2cc(O)cc(-c3ccc4c(N5CC6CCC(C5)N6)nc(OCC5(CN6CC[C@H](F)[C@H](F)C6)CC5)nc4c3F)c12 ; C#Cc1cccc2cc(O)cc(-c3ccc4c(N5CC6CCC(C5)N6)nc(OCC5(CN6CC[C@H](F)[C@@H](F)C6)CC5)nc4c3F)c12]

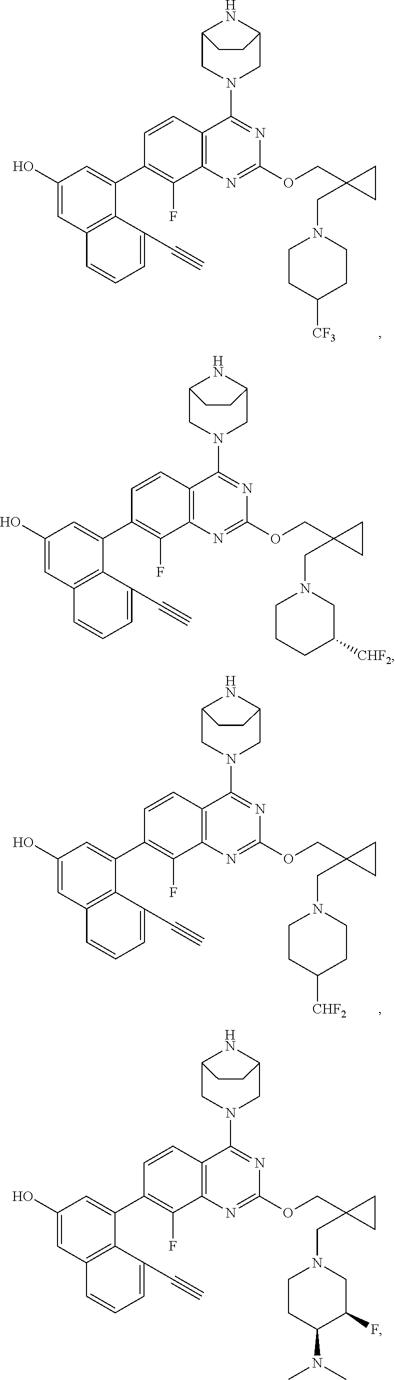 [SMILES: C#Cc1cccc2cc(O)cc(-c3ccc4c(N5CC6CCC(C5)N6)nc(OCC5(CN6CCC(C(F)(F)F)CC6)CC5)nc4c3F)c12 ; C#Cc1cccc2cc(O)cc(-c3ccc4c(N5CC6CCC(C5)N6)nc(OCC5(CN6CCC[C@@H](C(F)F)C6)CC5)nc4c3F)c12 ; C#Cc1cccc2cc(O)cc(-c3ccc4c(N5CC6CCC(C5)N6)nc(OCC5(CN6CCC(C(F)F)CC6)CC5)nc4c3F)c12 ; C#Cc1cccc2cc(O)cc(-c3ccc4c(N5CC6CCC(C5)N6)nc(OCC5(CN6CC[C@H](N(C)C)[C@H](F)C6)CC5)nc4c3F)c12]

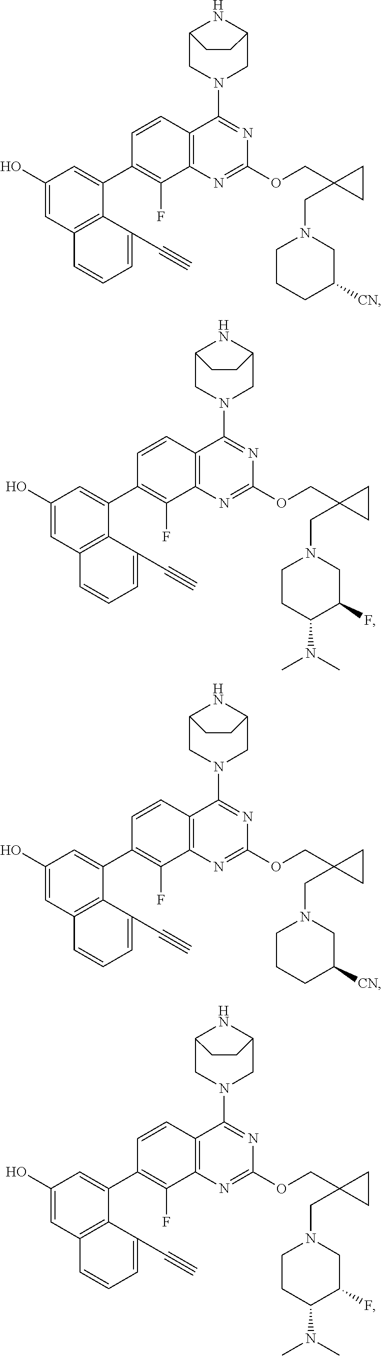 [SMILES: C#Cc1cccc2cc(O)cc(-c3ccc4c(N5CC6CCC(C5)N6)nc(OCC5(CN6CCC[C@@H](C#N)C6)CC5)nc4c3F)c12 ; C#Cc1cccc2cc(O)cc(-c3ccc4c(N5CC6CCC(C5)N6)nc(OCC5(CN6CC[C@@H](N(C)C)[C@H](F)C6)CC5)nc4c3F)c12 ; C#Cc1cccc2cc(O)cc(-c3ccc4c(N5CC6CCC(C5)N6)nc(OCC5(CN6CCC[C@H](C#N)C6)CC5)nc4c3F)c12 ; C#Cc1cccc2cc(O)cc(-c3ccc4c(N5CC6CCC(C5)N6)nc(OCC5(CN6CC[C@@H](N(C)C)[C@@H](F)C6)CC5)nc4c3F)c12]

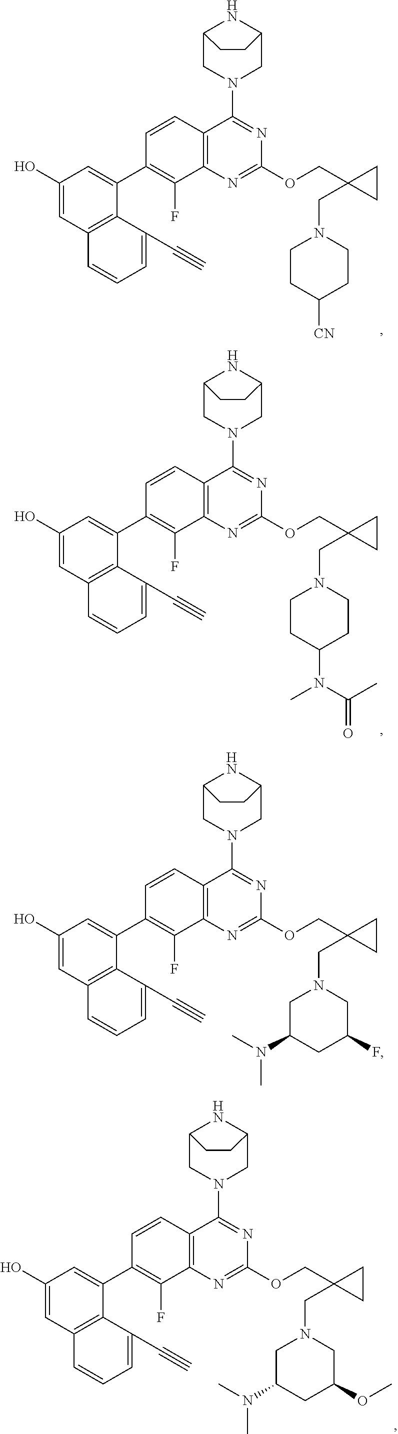 [SMILES: C#Cc1cccc2cc(O)cc(-c3ccc4c(N5CC6CCC(C5)N6)nc(OCC5(CN6CCC(C#N)CC6)CC5)nc4c3F)c12 ; C#Cc1cccc2cc(O)cc(-c3ccc4c(N5CC6CCC(C5)N6)nc(OCC5(CN6CCC(N(C)C(C)=O)CC6)CC5)nc4c3F)c12 ; C#Cc1cccc2cc(O)cc(-c3ccc4c(N5CC6CCC(C5)N6)nc(OCC5(CN6C[C@@H](F)C[C@@H](N(C)C)C6)CC5)nc4c3F)c12 ; C#Cc1cccc2cc(O)cc(-c3ccc4c(N5CC6CCC(C5)N6)nc(OCC5(CN6C[C@@H](OC)C[C@H](N(C)C)C6)CC5)nc4c3F)c12]

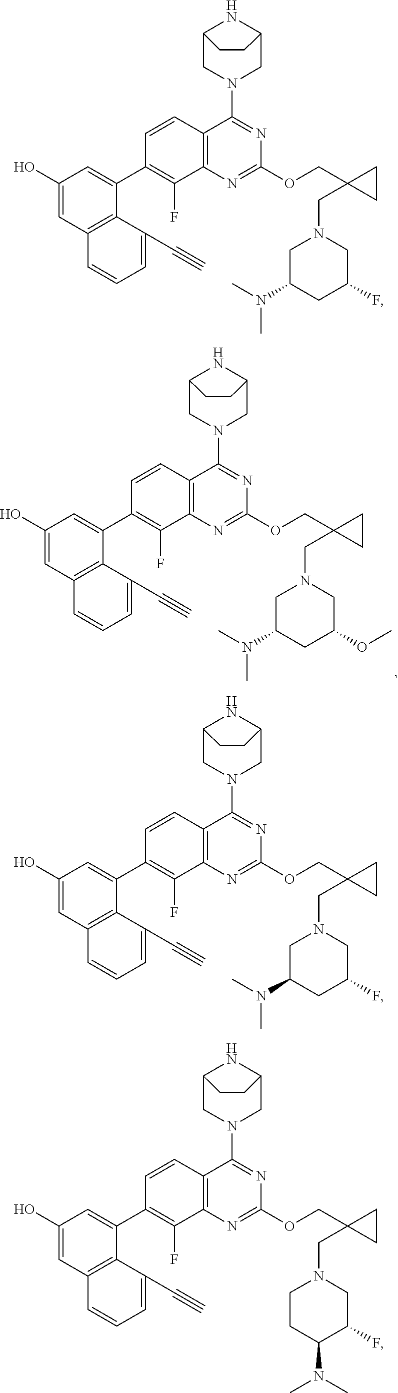

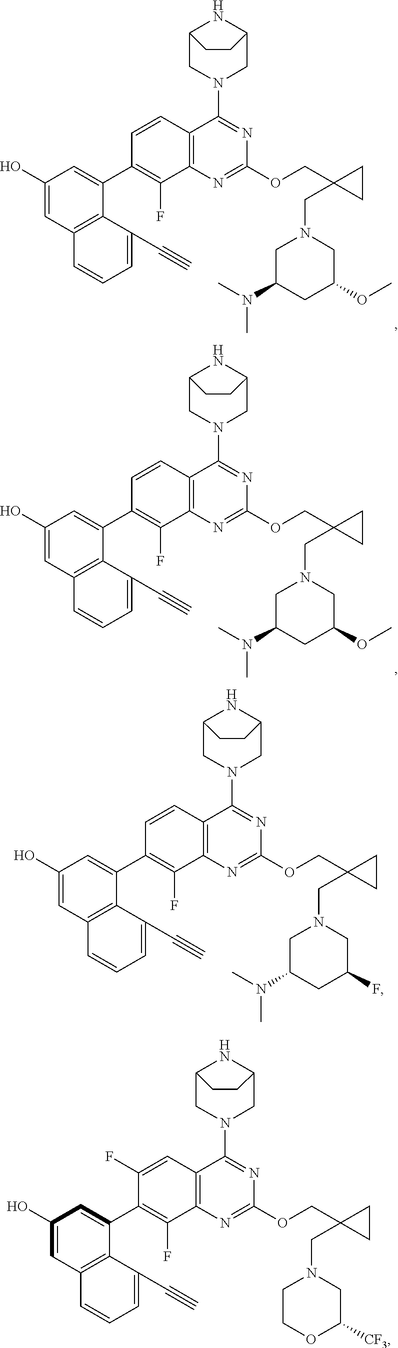

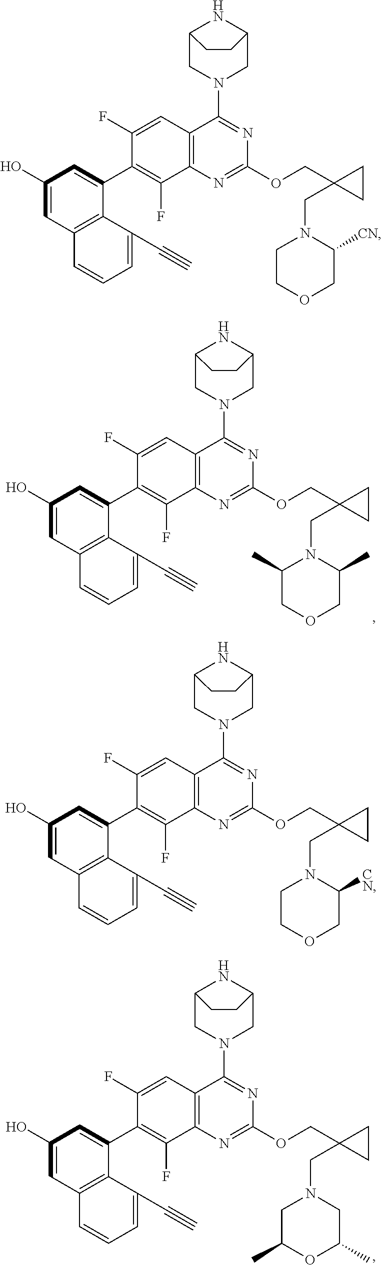

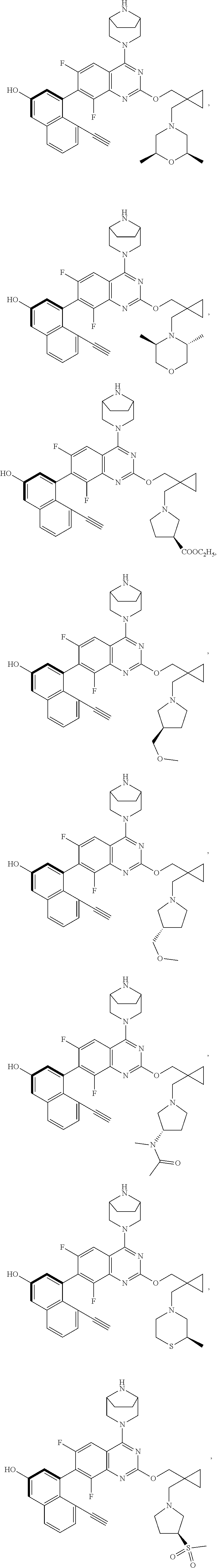

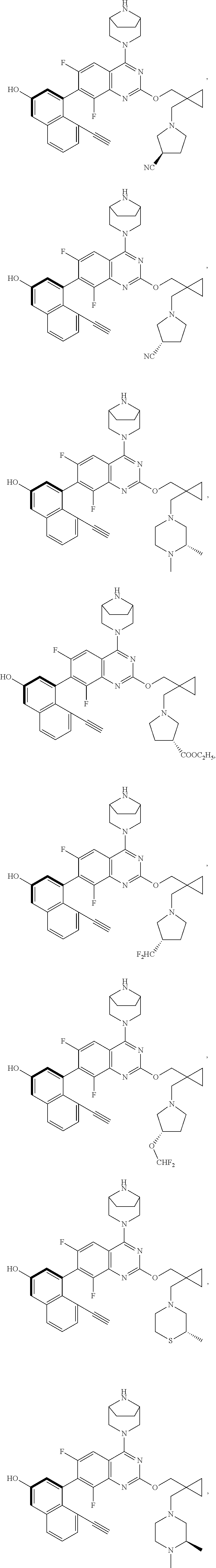

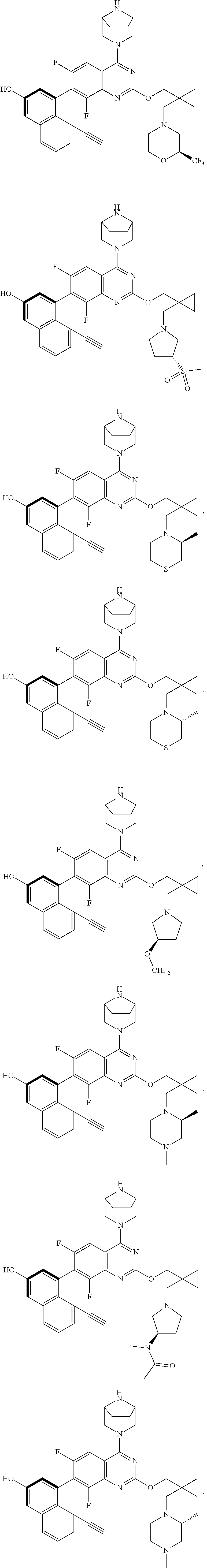

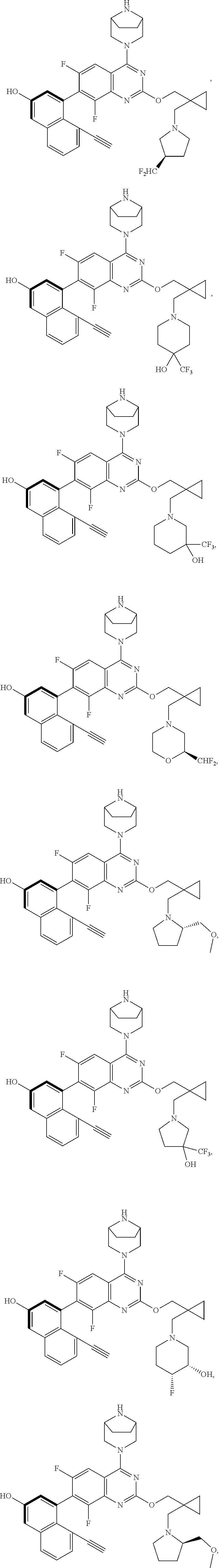

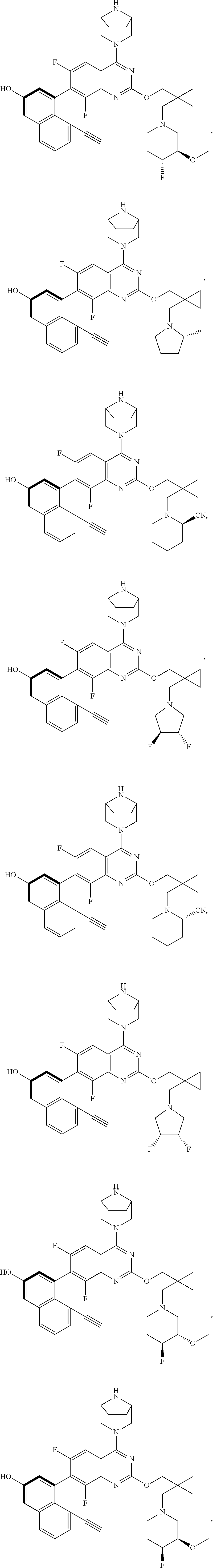

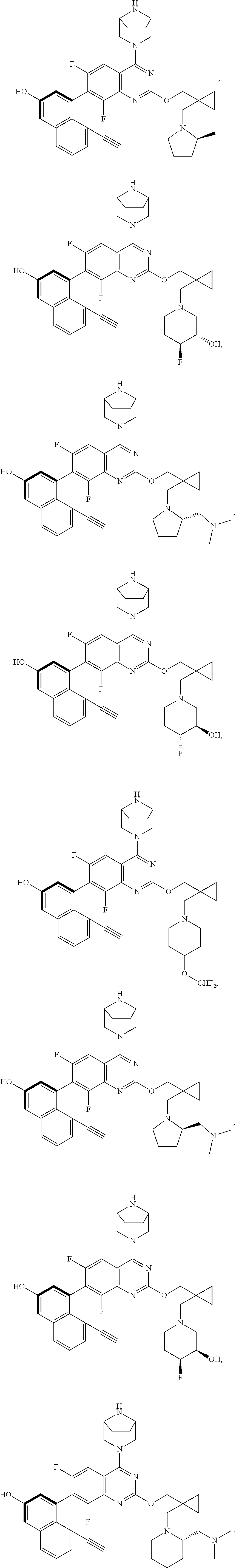

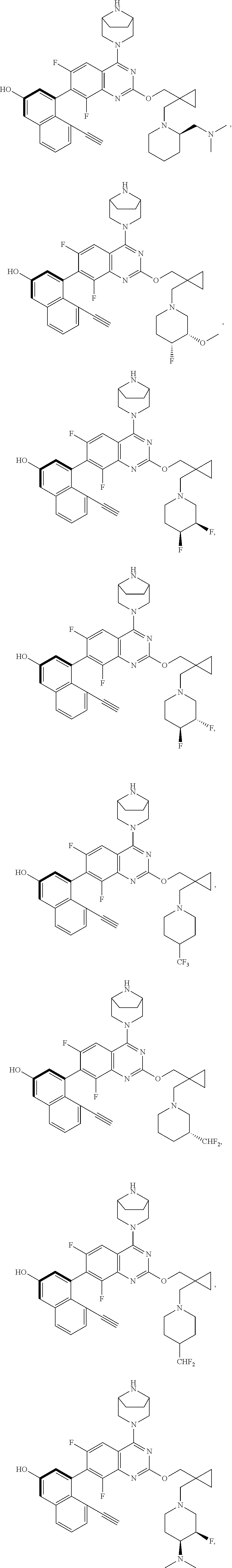

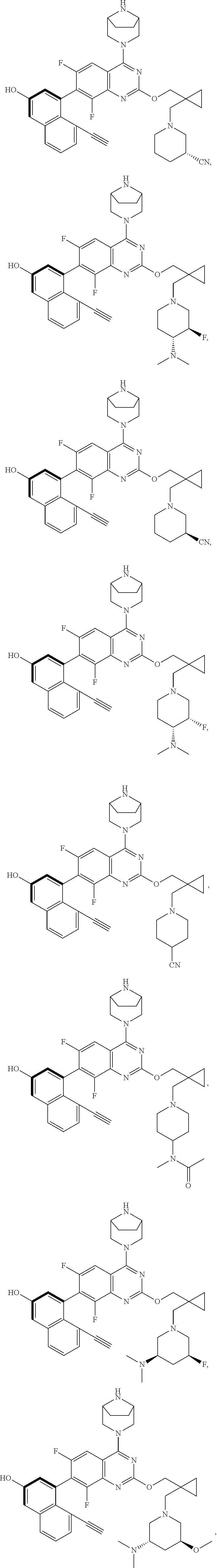

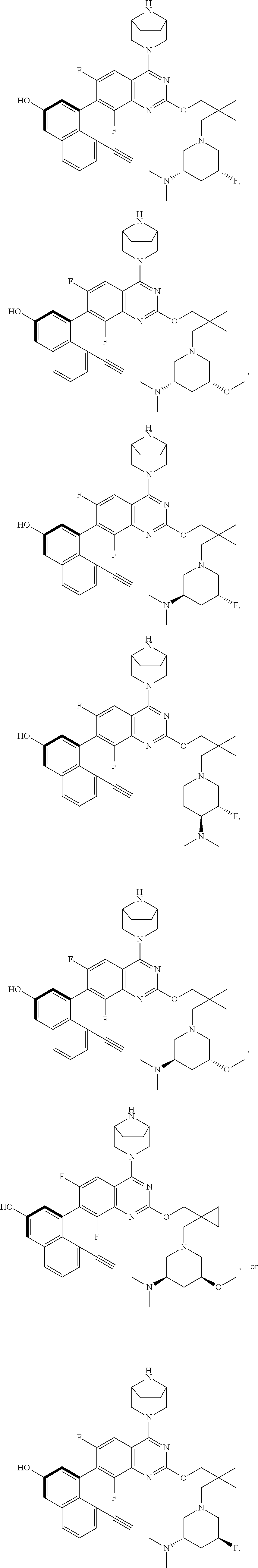

45. A pharmaceutical composition comprising a compound, a stereoisomer thereof, an atropisomer thereof, a deuterated derivative thereof, a tautomer thereof, or a pharmaceutically acceptable salt thereof of claim 27, and at least one pharmaceutically acceptable excipient.

46. A method of treating a subject having a diseases or conditions related to KRAS G12D mutant protein, said method comprising administering to the subject a therapeutically effective amount of a compound, a stereoisomer thereof, an atropisomer thereof, a deuterated derivative thereof, a tautomer thereof, or a pharmaceutically acceptable salt thereof of claim 27, wherein, the diseases or conditions related to KRAS G12D mutant protein is cancer related to KRAS G12D mutant protein, wherein the cancer is selected from pancreatic cancer, colorectal cancer, endometrial cancer or lung cancer, wherein the lung cancer is selected from non-small cell lung cancer or small cell lung cancer.
